(19) United States
(12) Patent Application Publication
(10) Pub. No.: US 2025/0176426 A1
(43) Pub. Date: May 29, 2025

(54) ORGANIC ELECTROLUMINESCENT DEVICE AND ORGANIC ELECTROLUMINESCENT COMPOUND FOR THE SAME

(71) Applicant: DuPont Specialty Materials Korea Ltd., Chungcheongnam-do (KR)

(72) Inventors: Mi-Ran SEO, Gyeonggi-do (KR); So-Young JUNG, Gyeonggi-do (KR); Tae-Jun HAN, Gyeonggi-do (KR); Bitnari KIM, Gyeonggi-do (KR); YeJin JEON, Gyeonggi-do (KR); So-Mi PARK, Gyeonggi-do (KR); Hyo-Jung LEE, Gyeonggi-do (KR); Hyun-Ju KANG, Gyeonggi-do (KR); Sang-Hee CHO, Gyeonggi-do (KR)

(21) Appl. No.: 18/956,177

(22) Filed: Nov. 22, 2024

(30) Foreign Application Priority Data
Nov. 29, 2023 (KR) 10-2023-0169060
Nov. 1, 2024 (KR) 10-2024-0153426

(51) Int. Cl. H10K 85/60; C07D 209/88; C07D 405/12; C09K 11/02; H10K 50/12; H10K 50/18

(52) U.S. Cl. CPC H10K 85/633; C07D 209/88; C07D 405/12; C09K 11/02; H10K 85/636; H10K 85/654; H10K 50/12; H10K 50/181; H10K 85/615; H10K 85/622; H10K 85/626; H10K 85/657; H10K 85/6572; H10K 85/6574; H10K 85/6576

(57) ABSTRACT

The present disclosure relates to an organic electroluminescent device. By comprising a compound and/or an organic electroluminescent material according to the present disclosure, an organic electroluminescent device exhibiting higher luminous efficiency and/or longer lifespan characteristics compared to conventional organic electroluminescent devices can be provided.

### TECHNICAL FIELD

The present disclosure relates to an organic electroluminescent device and an organic electroluminescent compound for the same.

### BACKGROUND ART

The TPD/Alq₃ bilayer small-molecule organic electroluminescent device (OLED) with green emission, which is constituted with a light-emitting layer and a charge transport layer, was first developed by Tang et al. of Eastman Kodak in 1987. Thereafter, studies on organic electroluminescent devices have proceeded rapidly, and OLEDs have since been commercialized. At present, OLEDs primarily use phosphorescent materials having excellent luminous efficiency in panel implementation. Therefore, an OLED having high luminous efficiency and/or long lifespan characteristics is required for long-term use and high display resolution.

Korean Patent No. 10-2306966 discloses an organic electroluminescent device comprising a compound having phenanthrooxazole or phenanthrothiazole as a main core and a phenanthro derivative as a host material, but does not specifically disclose an organic electroluminescent device having improved performance, such as high luminous efficiency and/or long lifespan characteristics by using a specific combination of compounds as an electron blocking layer and a host material for a light-emitting layer as in the present disclosure.

### DISCLOSURE OF THE INVENTION

### Technical Problem

The object of the present disclosure is to provide a compound effective for producing organic electroluminescent devices having high luminous efficiency and/or long lifespan characteristics and an organic electroluminescent material comprising the same.

### Solution to Problem

As a result of intensive studies to solve the technical problem above, the present inventors found that the aforementioned object can be achieved by an organic electroluminescent device comprising: a first electrode; a second electrode; a light-emitting layer between the first electrode and the second electrode; and at least one electron blocking layer between the first electrode and the light-emitting layer, wherein the electron blocking layer comprises a compound represented by the following Formula 1, and the light-emitting layer comprises at least two kinds of compounds, thereby completing the present invention.

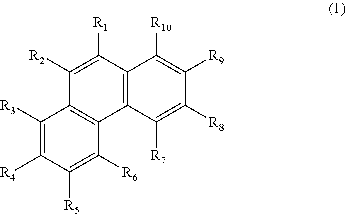

In Formula 1,
R₁ to R₁₀ each independently represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring of (C3-C30) aliphatic ring and (C6-C30) aromatic ring, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or *-L₁-N(Ar₁)(Ar₂); provided that at least one of R₁ to R₁₀ is *-L₁-N(Ar₁)(Ar₂);
L₁ represents a single bond, a substituted or unsubstituted (C6-C30)arylene, or a substituted or unsubstituted (3- to 30-membered)heteroarylene; and
Ar₁ and Ar₂ each independently represent a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl.

### Advantageous Effects of Invention

By comprising a compound according to the present disclosure and/or an organic electroluminescent material comprising the same, an organic electroluminescent device having high luminous efficiency and/or long lifespan properties can be provided.

### MODE FOR THE INVENTION

Hereinafter, the present disclosure will be described in detail. However, the following description is intended to explain the invention, and is not meant in any way to restrict the scope of the present disclosure.

The organic electroluminescent device according to the present disclosure comprises a first electrode; a second electrode; a light-emitting layer between the first electrode and the second electrode; and at least one electron blocking layer between the first electrode and the light-emitting layer, wherein the electron blocking layer comprises a compound represented by Formula 1, and the light-emitting layer comprises at least two kinds of compounds.

The organic electroluminescent device according to the present disclosure comprises at least one hole auxiliary layer between the first electrode and the electron blocking layer.

The light-emitting layer according to the present disclosure comprises at least one compound represented by Formula 2 and at least one compound represented by Formula 3.

The present disclosure provides an organic electroluminescent compound represented by Formula 1-1 as a compound for an organic electroluminescent device and an organic electroluminescent device comprising the same.

The present disclosure provides an organic electroluminescent compound represented by Formula 3′ as a compound for an organic electroluminescent device and an organic electroluminescent device comprising the same.

Herein, the term “organic electroluminescent compound” in the present disclosure means a compound that may be used in an organic electroluminescent device, and this may be comprised in any material layer constituting an organic electroluminescent device, as necessary.

Herein, the term “organic electroluminescent material” means a material that may be used in an organic electroluminescent device, and this may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (containing host and dopant materials), an electron buffer material, a hole blocking material, an electron transport material, or an electron injection material, etc.

The term “a plurality of organic electroluminescent materials” in the present disclosure means an organic electroluminescent material comprising a combination of at least two compounds, which may be comprised in any layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of organic electroluminescent materials may be a combination of at least two compounds, which may be comprised in at least one layer of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. As such, at least two compounds may be comprised in the same layer or in different layers, and may be mixture-evaporated or co-evaporated, or may be individually evaporated.

Herein, the term “a plurality of host materials” means an organic electroluminescent material comprising a combination of at least two host materials. It may mean both a material before being comprised in an organic electroluminescent device (e.g., before vapor deposition) and a material after being comprised in an organic electroluminescent device (e.g., after vapor deposition). A plurality of host materials of the present disclosure may be comprised in any light-emitting layer constituting an organic electroluminescent device. The at least two compounds comprised in a plurality of host materials may be comprised together in one light-emitting layer, or may each be comprised in separate light-emitting layers. When at least two compounds are comprised in one light-emitting layer, the at least two compounds may be mixture-evaporated to form a layer or may be individually and simultaneously co-evaporated to form a layer.

Herein, “(C1-C30)alkyl(ene)” is meant to be a linear or branched alkyl having 1 to 30 carbon atoms constituting the chain, in which the number of carbon atoms is preferably 1 to 20, more preferably 1 to 10. The above alkyl may include methyl, ethyl, n-propyl, isopropyl, n-butyl, isobutyl, tert-butyl, sec-butyl, etc. Herein, “(C3-C30)cycloalkyl(ene)” is meant to be a mono- or polycyclic hydrocarbon having 3 to 30 ring backbone carbon atoms, in which the number of carbon atoms is preferably 3 to 20, more preferably 3 to 7. The above cycloalkyl may include cyclopropyl, cyclobutyl, cyclopentyl, cyclohexyl, cyclopentylmethyl, cyclohexylmethyl, etc. The “(3- to 7-membered)heterocycloalkyl” in the present disclosure is meant to be a cycloalkyl having 3 to 7 ring backbone atoms, preferably 5 to 7 ring backbone atoms and including at least one heteroatom selected from the group consisting of B, N, O, S, Si, and P, preferably O, S, and N, and includes tetrahydrofuran, pyrrolidine, thiolan, tetrahydropyran, etc. The “(C6-C30)aryl(ene)” in the present disclosure is meant to be a monocyclic or fused ring radical derived from an aromatic hydrocarbon having 6 to 30 ring backbone carbon atoms, in which the number of the ring backbone carbon atoms is preferably 6 to 20, more preferably 6 to 15. The above aryl may be partially saturated and may comprise a spiro structure. Examples of the aryl specifically include phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluorene-fluorene]yl, spiro[fluorene-benzofluorene]yl, azulenyl, tetramethyl-dihydrophenanthrenyl, etc. More specifically, the aryl may be o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenyl, 4″-t-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc. The “(3-to 30-membered)heteroaryl(ene)” in the present disclosure is an aryl having 3 to 30 ring backbone atoms and including at least one heteroatom selected from the group consisting of B, N, O, S, Si, P, Se, and Ge in which the number of the ring backbone atoms is preferably 5 to 25. The number of the heteroatoms in the heteroaryl is preferably 1 to 4. The above heteroaryl may be a monocyclic ring, or a fused ring condensed with at least one benzene ring, and may be partially saturated. Also, the above heteroaryl herein may be one formed by linking at least one heteroaryl or aryl group to a heteroaryl group via a single bond(s). Examples of the heteroaryl specifically may include a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, dibenzoselenophenyl, benzofuroquinolinyl, benzofuroquinazolinyl, benzofuronaphthiridinyl, benzofuropyrimidinyl, naphthofuropyrimidinyl, benzothienoquinolinyl, benzothienoquinazolinyl, benzothienonaphthiridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzoimidazolyl, benzothiazolyl, benzoisothiazolyl, benzoisoxazolyl, benzoxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acridinyl, silafluorenyl, germafluorenyl, benzotriazolyl, phenazinyl, imidazopyridinyl, chromenoquinazolinyl, thiochromenoquinazolinyl, dimethylbenzopyrimidinyl, indolocarbazolyl, indenocarbazolyl, etc. More specifically, the heteroaryl may be 1-pyrrolyl, 2-pyrrolyl, 3-pyrrolyl, 2-pyridinyl, 3-pyridinyl, 4-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolizidinyl, 2-indolizidinyl, 3-indolizidinyl, 5-indolizidinyl, 6-indolizidinyl, 7-indolizidinyl, 8-indolizidinyl, 2-imidazopyridinyl, 3-imidazopyridinyl, 5-imidazopyridinyl, 6-imidazopyridinyl, 7-imidazopyridinyl, 8-imidazopyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-t-butylpyrrol-4-yl, 3-(2-phenylpropyl) pyrrol-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Additionally, “heteroaryl(ene)” can be classified as a heteroaryl(ene) with electronic properties or a heteroaryl(ene) with hole properties. A heteroaryl(ene) with electronic properties is a substituent with relatively abundant electrons in the parent nucleus, and for example, it may be a substituted or unsubstituted pyridinyl, a substituted or unsubstituted pyrimidinyl, a substituted or unsubstituted triazinyl, a substituted or unsubstituted quinazolinyl, a substituted or unsubstituted quinoxalinyl, a substituted or unsubstituted quinolyl, etc. A heteroaryl(ene), which has hole properties, is a substituent with a relative lack of electrons in the parent nucleus, and for example, it may be a substituted or unsubstituted carbazolyl, a substituted or unsubstituted dibenzofuranyl, or a substituted or unsubstituted dibenzothiophenyl. Herein, “a fused ring of (C3-C30) aliphatic ring and (C6-C30) aromatic ring” means a ring formed by fusing at least one aliphatic ring having 3 to 30 ring backbone carbon atoms in which the number of carbon atoms is preferably 3 to 25, more preferably 3 to 18, and at least one aromatic ring having 6 to 30 ring backbone carbon atoms in which the number of carbon atoms is preferably 6 to 25, more preferably 6 to 18. For example, the fused ring may be a fused ring of at least one benzene and at least one cyclohexane, or a fused ring of at least one naphthalene and at least one cyclopentane, etc. Herein, the carbon atoms in the fused ring of (C3-C30) aliphatic ring and (C6-C30) aromatic ring may be replaced with at least one heteroatom selected from B, N, O, S, Si, and P, preferably at least one heteroatom selected from N, O, and S. The “halogen” in the present disclosure includes F, Cl, Br, and I.

In addition, “ortho-” (“o-”), “meta-” (“m-”), and “para-” (“p-”) are meant to signify the substitution position of all substituents. An ortho-configuration describes a compound with substituents which are adjacent to each other, e.g., at the 1 and 2 positions on benzene. A meta-configuration indicates the next substitution position of the immediately adjacent substitution position, e.g., a compound with substituents at the 1 and 3 positions on benzene. A para-configuration indicates the next substitution position from the meta-position, e.g., a compound with substituents at the 1 and 4 positions on benzene.

Herein, “a ring formed in linking to an adjacent substituent” means a substituted or unsubstituted (3- to 30-membered) mono- or polycyclic, alicyclic, aromatic ring, or a combination thereof, formed by linking or fusing two or more adjacent substituents, and preferably this may be a substituted or unsubstituted (3- to 26-membered) mono- or polycyclic, alicyclic, aromatic ring, or a combination thereof. Further, the formed ring may include at least one heteroatom selected from the group consisting of B, N, O, S, Si, and P, preferably N, O, and S. According to one embodiment of the present disclosure, the number of ring backbone atoms is 5 to 20; according to another embodiment of the present disclosure, the number of ring backbone atoms is 5 to 15. In one embodiment, the fused ring may be, for example, a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, or a substituted or unsubstituted carbazole ring, etc.

In addition, the term “substituted” in the expression “substituted or unsubstituted” means that a hydrogen atom in a certain functional group is replaced with another atom or functional group, i.e., a substituent. Unless otherwise specified, the substituents may not be limited to hydrogen at positions where the substituents may be substituted, and when two or more hydrogen atoms are each replaced with a substituent in a functional group, the substituents may be the same as or different from each other. It also includes that the hydrogen atom is replaced with a group formed by a linkage of two or more substituents of the above substituents. For example, the “group formed by a linkage of two or more substituents” may be pyridine-triazine. That is, pyridine-triazine may be heteroaryl or may be interpreted as one substituent in which two heteroaryls are connected. For example, in the present disclosure, “phenylnaphthyl” means naphthalene substituted with a phenyl group, and “naphthylphenyl” means benzene substituted with a naphthyl group. Preferably, the substituted alkyl, the substituted alkenyl, the substituted cycloalkyl, the substituted heterocycloalkyl, the substituted aryl(ene), the substituted heteroaryl(ene), the substituted alkoxy, the substituted trialkylsilyl, the substituted dialkylarylsilyl, the substituted alkyldiarylsilyl, the substituted triarylsilyl, the substituted mono- or di-alkylamino, the substituted mono- or di-arylamino, the substituted alkylarylamino, and the substituted fused ring of aliphatic ring and aromatic ring in the formulas of the present disclosure each independently may be substituted with least one selected from the group consisting of: deuterium; halogen; cyano; carboxyl; nitro; hydroxyl; (C1-C30)alkyl; halo (C1-C30)alkyl; (C2-C30)alkenyl; (C2-C30)alkynyl; (C1-C30)alkoxy; (C1-C30)alkylthio; (C3-C30)cycloalkyl; (C3-C30)cycloalkenyl; (3- to 7-membered)heterocycloalkyl; (C6-C30)aryloxy; (C6-C30)arylthio; (3-to 30-membered)heteroaryl unsubstituted or substituted with at least one (C6-C30)aryl; (C6-C30)aryl unsubstituted or substituted with at least one of (C1-C30)alkyl and (3- to 30-membered)heteroaryl; tri(C1-C30)alkylsilyl; tri(C6-C30)arylsilyl; di(C1-C30)alkyl(C6-C30)arylsilyl; (C1-C30)alkyldi(C6-C30)arylsilyl; tri(C6-C30)arylgermanyl; amino; mono- or di-(C1-C30)alkylamino; mono- or di-(C2-C30)alkenylamino; mono- or di-(C6-C30)arylamino unsubstituted or substituted with (C1-C30)alkyl; mono- or di-(3- to 30-membered)heteroarylamino; (C1-C30)alkyl(C2-C30)alkenylamino; (C1-C30)alkyl(C6-C30)arylamino; (C1-C30)alkyl (3- to 30-membered)heteroarylamino; (C2-C30)alkenyl (C6-C30)arylamino; (C2-C30)alkenyl (3- to 30-membered)heteroarylamino; (C6-C30)aryl (3- to 30-membered)heteroarylamino; (C1-C30)alkylcarbonyl; (C1-C30)alkoxycarbonyl; (C6-C30)arylcarbonyl; di(C6-C30)arylboronyl; di(C1-C30)alkylboronyl; (C1-C30)alkyl(C6-C30)arylboronyl; (C6-C30)ar(C1-C30)alkyl; and (C1-C30)alkyl(C6-C30)aryl etc. For example, the substituents may be substituted with deuterium, cyano, methyl, phenyl, naphthyl, triphenylsilyl, pyridyl, dibenzofuranyl, or dibenzothiophenyl, etc.

When a substituent is not shown in the chemical formula or the compound structure of the present disclosure, it may signify that all positions that may be present as substituents are hydrogen or deuterium. That is, in the case of deuterium, an isotope of hydrogen, some of the hydrogen atoms may be deuterium, which is an isotope; and in this case, the content of deuterium may be 0% to 100%. In the case where a substituent is not shown in the chemical formula or the compound structure of the present disclosure, when deuterium is not explicitly excluded, hydrogen and deuterium may be mixed and used in the compound, such as when the content of deuterium is 0%, the content of hydrogen is 100%, and all substituents are hydrogen. The deuterium is an element having a deuteron composed of one proton and one neutron as an atomic nucleus, which is one of the isotopes of hydrogen, and may be represented by hydrogen-2, and the element symbol may be D or 2H. The isotope having the same atomic number (Z) and a different mass number (A) may also be interpreted as an element having the same number of protons and the different number of neutron.

Herein, “combinations thereof” signifies that one or more components of the corresponding list are combined to form a known or chemically stable arrangement that a person skilled in the art could conceive of from the corresponding list. For example, alkyl and deuterium may be combined to form partially or entirely deuterated alkyl groups; halogen and alkyl may be combined to form halogenated alkyl substituents; and halogen, alkyl, and aryl may be combined to form halogenated arylalkyl. For example, preferred combinations of substituents may include up to 50 atoms excluding hydrogen and deuterium, or include up to 40 atoms excluding hydrogen and deuterium, or include up to 30 atoms excluding hydrogen and deuterium, or in many cases, preferred combinations of substituents may include up to 20 atoms excluding hydrogen and deuterium.

In the formulas of the present disclosure, when multiple substituents are indicated by the same symbol, each of these substituents represented by the same symbol may be the identical or different from one another.

Hereinafter, the organic electroluminescent device according to one embodiment will be described in detail.

The organic electroluminescent device according to the present disclosure comprises: a first electrode; a second electrode; a light-emitting layer between the first electrode and the second electrode; and at least one electron blocking layer between the first electrode and the light-emitting layer, wherein the electron blocking layer comprises a compound represented by the following Formula 1, and the light-emitting layer comprises at least two kinds of compounds.

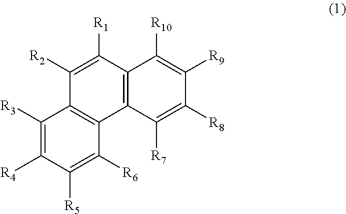

In Formula 1,
R₁ to R₁₀ each independently represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring of (C3-C30) aliphatic ring and (C6-C30)aromatic ring, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or *-L₁-N(Ar₁)(Ar₂); provided that at least one of R₁ to R₁₀ is *-L₁-N(Ar₁)(Ar₂);
L₁ represents a single bond, a substituted or unsubstituted (C6-C30)arylene, or a substituted or unsubstituted (3- to 30-membered)heteroarylene; and
Ar₁ and Ar₂ each independently represent a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl.

In one embodiment, R₁ to R₁₀, which are not -L₁-N(Ar₁)(Ar₂), each independently may be hydrogen, deuterium, a halogen, a cyano, a substituted or unsubstituted (C6-C30)aryl, or a substituted or unsubstituted (5- to 30-membered)heteroaryl, preferably hydrogen, deuterium, a substituted or unsubstituted (C6-C25)aryl, or a substituted or unsubstituted (5- to 25-membered)heteroaryl, more preferably hydrogen, deuterium, a substituted or unsubstituted (C6-C18)aryl, or a substituted or unsubstituted (5- to 18-membered)heteroaryl. For example, R₁ to R₁₀ each independently may be hydrogen, deuterium, a phenyl unsubstituted or unsubstituted with a cyano, a naphthyl unsubstituted or unsubstituted with a phenyl, a substituted or unsubstituted p-biphenyl, a substituted or unsubstituted m-biphenyl, a substituted or unsubstituted m-terphenyl, a substituted or unsubstituted phenanthrenyl, a substituted or unsubstituted triphenylenyl, a substituted or unsubstituted pyridyl, a substituted or unsubstituted quinolyl, or a substituted or unsubstituted dibenzofuranyl. In one embodiment, L₁ may be a single bond or a substituted or unsubstituted (C6-C30)arylene, preferably a single bond or a substituted or unsubstituted (C6-C25)arylene, more preferably a single bond or a substituted or unsubstituted (C6-C18)arylene. For example, L₁ may be a single bond, a substituted or unsubstituted phenylene, a substituted or unsubstituted naphthylene, a substituted or unsubstituted p-biphenylene, a substituted or unsubstituted m-biphenylene, or a substituted or unsubstituted o-biphenylene.

In one embodiment, Ar₁ and Ar₂ each independently may be a substituted or unsubstituted (C6-C30)aryl or a substituted or unsubstituted (5- to 30-membered)heteroaryl, preferably a substituted or unsubstituted (C6-C25)aryl or a substituted or unsubstituted (5-to 25-membered)heteroaryl, more preferably a substituted or unsubstituted (C6-C18)aryl or a substituted or unsubstituted (5- to 18-membered)heteroaryl. For example, Ar₁ and Ar₂ each independently may be a substituted or unsubstituted phenyl, a substituted or unsubstituted naphthyl, a substituted or unsubstituted p-biphenyl, a substituted or unsubstituted o-biphenyl, a substituted or unsubstituted m-terphenyl, a substituted or unsubstituted p-terphenyl, a substituted or unsubstituted o-terphenyl, a substituted or unsubstituted dimethylfluorenyl, a substituted or unsubstituted diethylfluorenyl, a substituted or unsubstituted phenanthrenyl, a substituted or unsubstituted dibenzofuranyl, a substituted or unsubstituted dibenzothiophenyl, a substituted or unsubstituted carbazolyl, or a substituted or unsubstituted benzonaphthothiophenyl. Wherein, the substituents, for example, may be substituted with at least one of deuterium, tert-butyl, amino, phenyl, p-biphenyl, m-terphenyl, fluorenyl, triphenylsilyl, naphthyl, phenanthrenyl, pyridyl unsubstituted or substituted with phenyl, dibenzothiophenyl, dibenzofuranyl, and carbazolyl.

According to one embodiment, the compound represented by Formula 1 may be more specifically illustrated by the following compounds, but is not limited thereto:

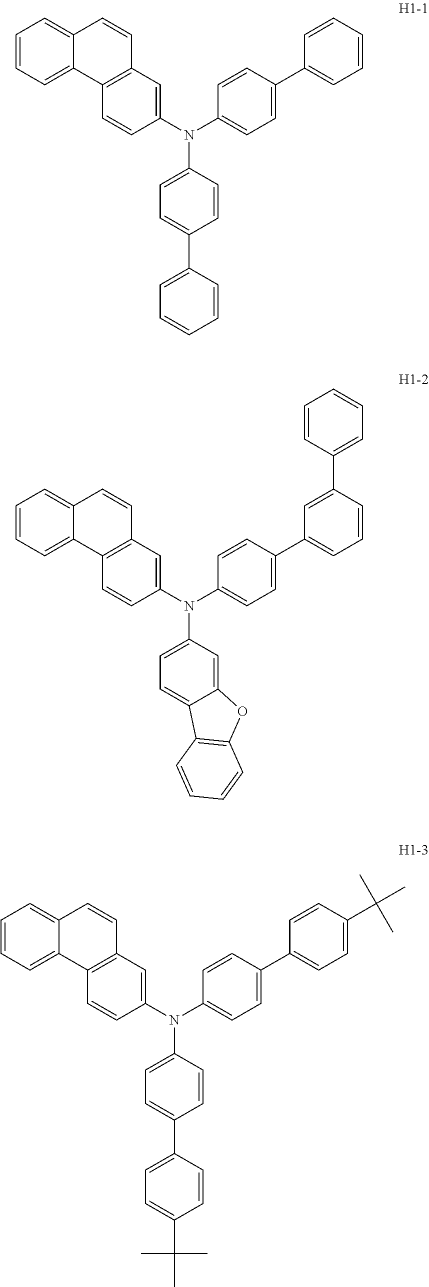 [SMILES: c1ccc(-c2ccc(N(c3ccc(-c4ccccc4)cc3)c3ccc4c(ccc5ccccc54)c3)cc2)cc1 ; c1ccc(-c2cccc(-c3ccc(N(c4ccc5c(ccc6ccccc65)c4)c4ccc5c(c4)oc4ccccc45)cc3)c2)cc1 ; CC(C)(C)c1ccc(-c2ccc(N(c3ccc(-c4ccc(C(C)(C)C)cc4)cc3)c3ccc4c(ccc5ccccc54)c3)cc2)cc1]

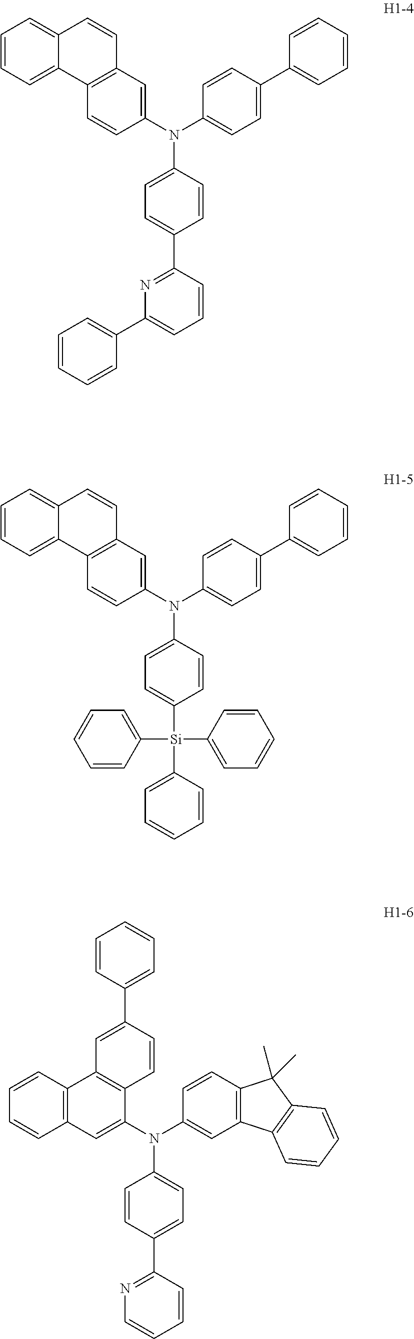 [SMILES: c1ccc(-c2ccc(N(c3ccc(-c4cccc(-c5ccccc5)n4)cc3)c3ccc4c(ccc5ccccc54)c3)cc2)cc1 ; c1ccc(-c2ccc(N(c3ccc([Si](c4ccccc4)(c4ccccc4)c4ccccc4)cc3)c3ccc4c(ccc5ccccc54)c3)cc2)cc1 ; CC1(C)c2ccccc2-c2cc(N(c3ccc(-c4ccccn4)cc3)c3cc4ccccc4c4cc(-c5ccccc5)ccc34)ccc21]

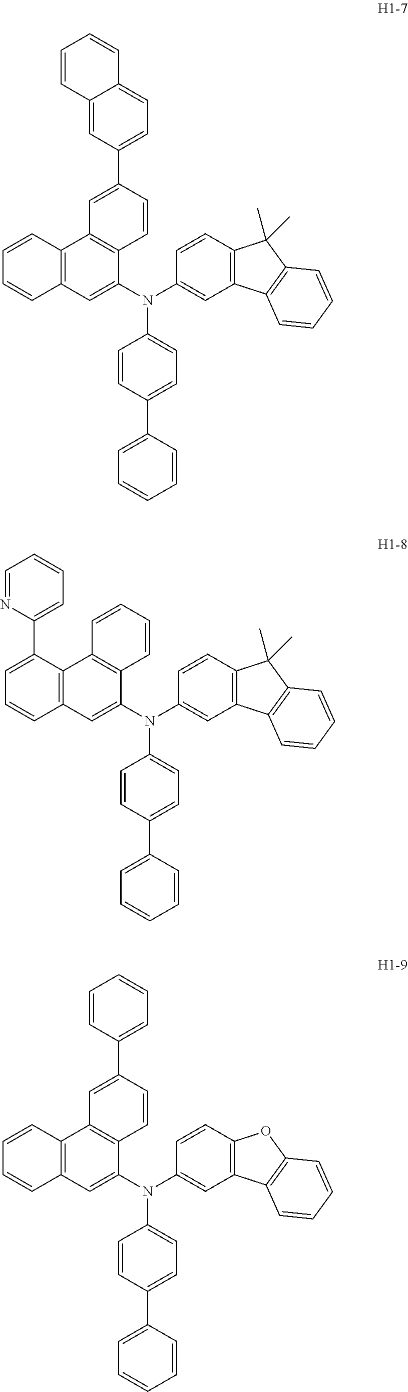 [SMILES: CC1(C)c2ccccc2-c2cc(N(c3ccc(-c4ccccc4)cc3)c3cc4ccccc4c4cc(-c5ccc6ccccc6c5)ccc34)ccc21 ; CC1(C)c2ccccc2-c2cc(N(c3ccc(-c4ccccc4)cc3)c3cc4cccc(-c5ccccn5)c4c4ccccc34)ccc21 ; c1ccc(-c2ccc(N(c3ccc4oc5ccccc5c4c3)c3cc4ccccc4c4cc(-c5ccccc5)ccc34)cc2)cc1]

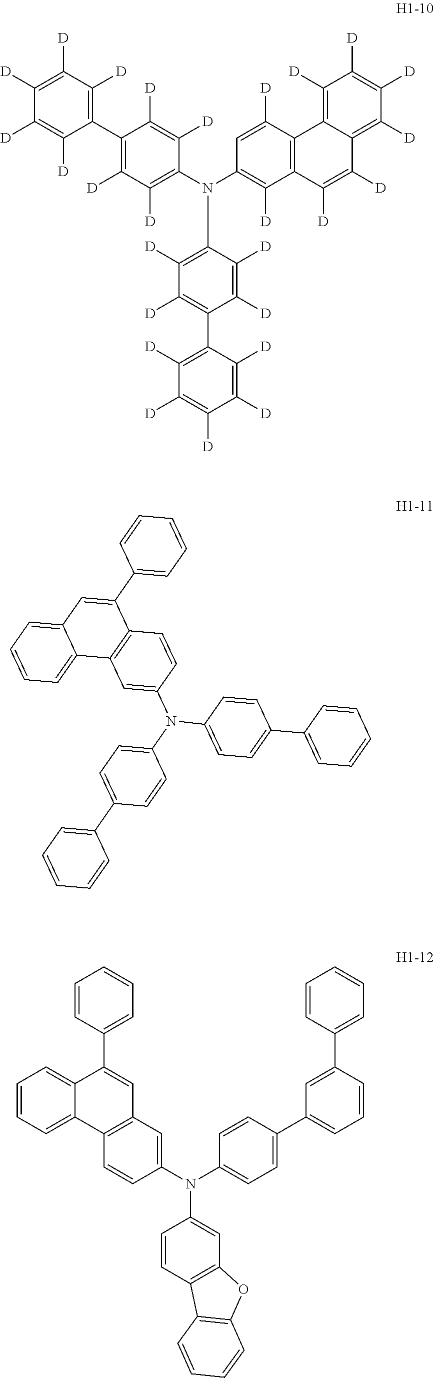 [SMILES: [2H]c1c([2H])c([2H])c(-c2c([2H])c([2H])c(N(c3cc([2H])c4c(c3[2H])c([2H])c([2H])c3c([2H])c([2H])c([2H])c([2H])c34)c3c([2H])c([2H])c(-c4c([2H])c([2H])c([2H])c([2H])c4[2H])c([2H])c3[2H])c([2H])c2[2H])c([2H])c1[2H] ; c1ccc(-c2ccc(N(c3ccc(-c4ccccc4)cc3)c3ccc4c(-c5ccccc5)cc5ccccc5c4c3)cc2)cc1 ; c1ccc(-c2cccc(-c3ccc(N(c4ccc5c(c4)cc(-c4ccccc4)c4ccccc45)c4ccc5c(c4)oc4ccccc45)cc3)c2)cc1]

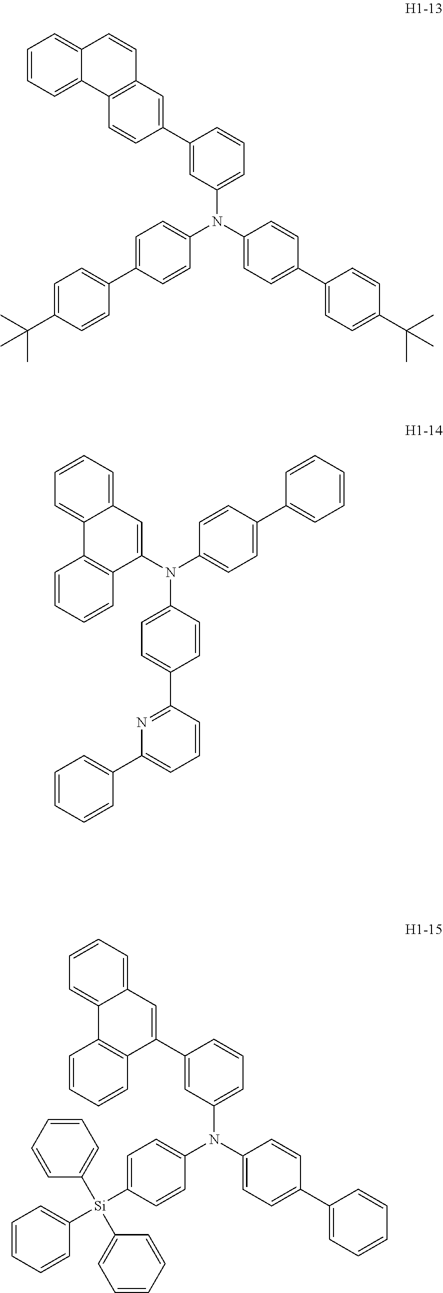 [SMILES: CC(C)(C)c1ccc(-c2ccc(N(c3ccc(-c4ccc(C(C)(C)C)cc4)cc3)c3cccc(-c4ccc5c(ccc6ccccc65)c4)c3)cc2)cc1 ; c1ccc(-c2ccc(N(c3ccc(-c4cccc(-c5ccccc5)n4)cc3)c3cc4ccccc4c4ccccc34)cc2)cc1 ; c1ccc(-c2ccc(N(c3ccc([Si](c4ccccc4)(c4ccccc4)c4ccccc4)cc3)c3cccc(-c4cc5ccccc5c5ccccc45)c3)cc2)cc1]

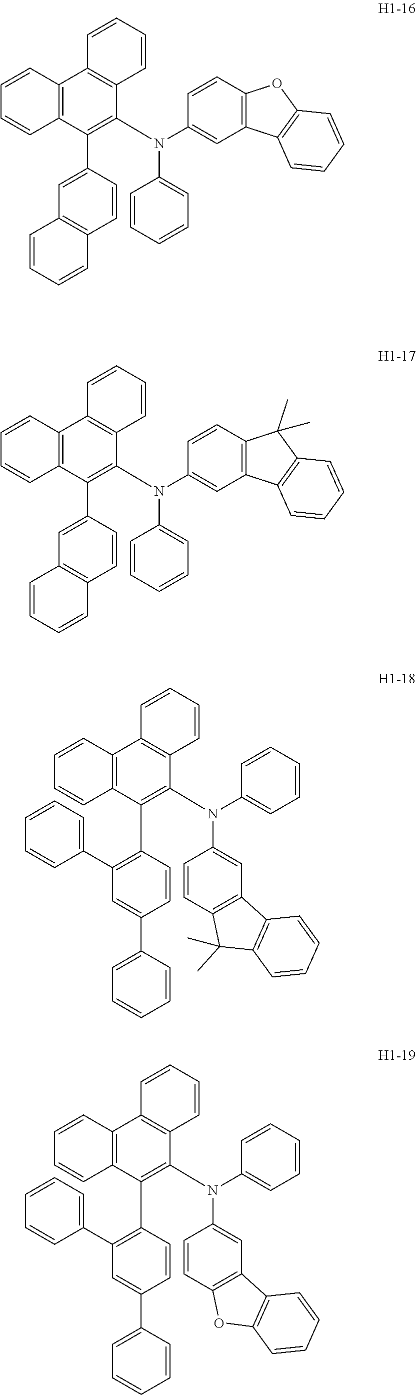 [SMILES: c1ccc(N(c2ccc3oc4ccccc4c3c2)c2c(-c3ccc4ccccc4c3)c3ccccc3c3ccccc23)cc1 ; CC1(C)c2ccccc2-c2cc(N(c3ccccc3)c3c(-c4ccc5ccccc5c4)c4ccccc4c4ccccc34)ccc21 ; CC1(C)c2ccccc2-c2cc(N(c3ccccc3)c3c(-c4ccc(-c5ccccc5)cc4-c4ccccc4)c4ccccc4c4ccccc34)ccc21 ; c1ccc(-c2ccc(-c3c(N(c4ccccc4)c4ccc5oc6ccccc6c5c4)c4ccccc4c4ccccc34)c(-c3ccccc3)c2)cc1]

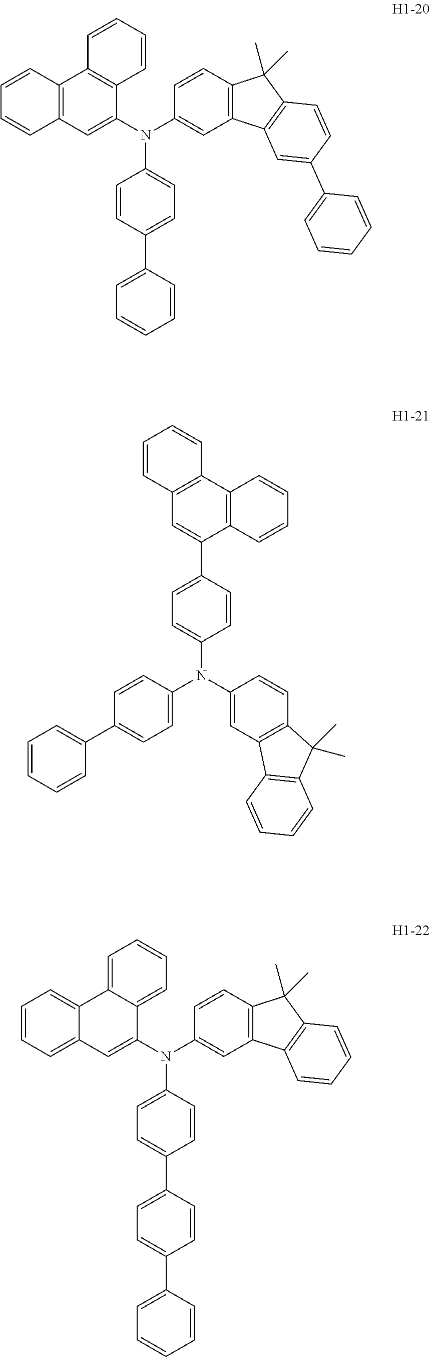 [SMILES: CC1(C)c2ccc(-c3ccccc3)cc2-c2cc(N(c3ccc(-c4ccccc4)cc3)c3cc4ccccc4c4ccccc34)ccc21 ; CC1(C)c2ccccc2-c2cc(N(c3ccc(-c4ccccc4)cc3)c3ccc(-c4cc5ccccc5c5ccccc45)cc3)ccc21 ; CC1(C)c2ccccc2-c2cc(N(c3ccc(-c4ccc(-c5ccccc5)cc4)cc3)c3cc4ccccc4c4ccccc34)ccc21]

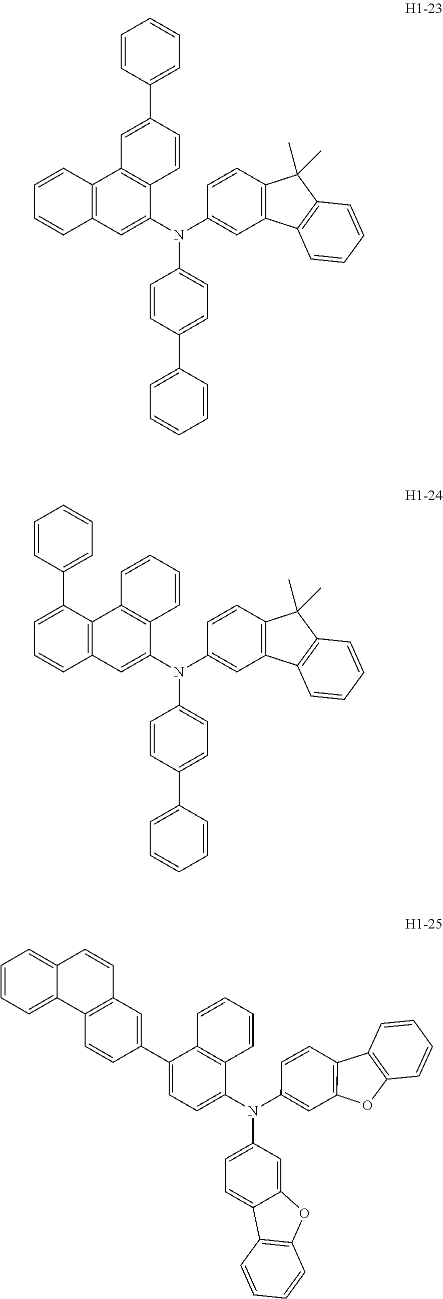 [SMILES: CC1(C)c2ccccc2-c2cc(N(c3ccc(-c4ccccc4)cc3)c3cc4ccccc4c4cc(-c5ccccc5)ccc34)ccc21 ; CC1(C)c2ccccc2-c2cc(N(c3ccc(-c4ccccc4)cc3)c3cc4cccc(-c5ccccc5)c4c4ccccc34)ccc21 ; c1ccc2c(c1)ccc1cc(-c3ccc(N(c4ccc5c(c4)oc4ccccc45)c4ccc5c(c4)oc4ccccc45)c4ccccc34)ccc12]

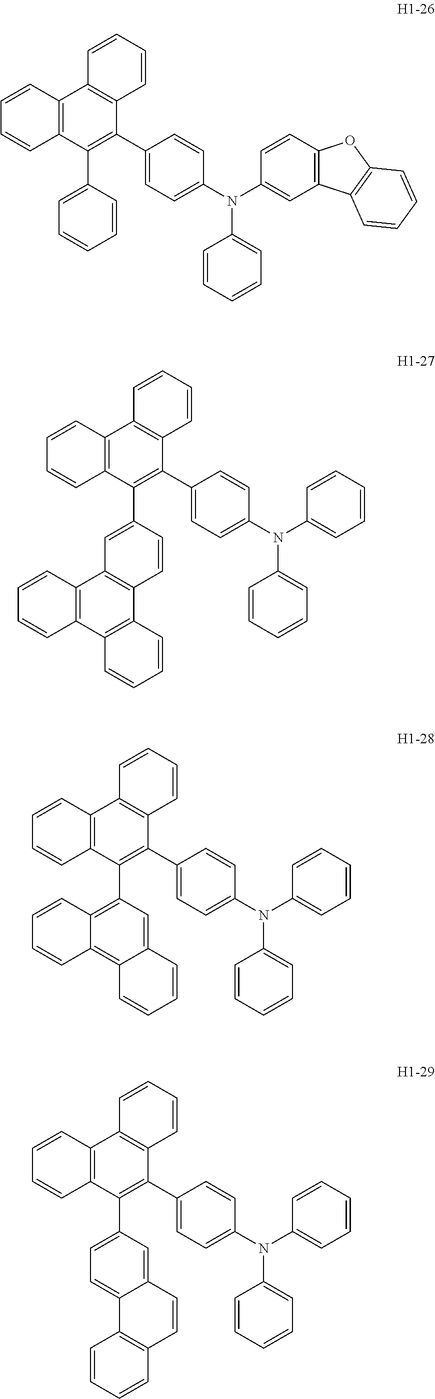 [SMILES: c1ccc(-c2c(-c3ccc(N(c4ccccc4)c4ccc5oc6ccccc6c5c4)cc3)c3ccccc3c3ccccc23)cc1 ; c1ccc(N(c2ccccc2)c2ccc(-c3c(-c4ccc5c6ccccc6c6ccccc6c5c4)c4ccccc4c4ccccc34)cc2)cc1 ; c1ccc(N(c2ccccc2)c2ccc(-c3c(-c4cc5ccccc5c5ccccc45)c4ccccc4c4ccccc34)cc2)cc1 ; c1ccc(N(c2ccccc2)c2ccc(-c3c(-c4ccc5c(ccc6ccccc65)c4)c4ccccc4c4ccccc34)cc2)cc1]

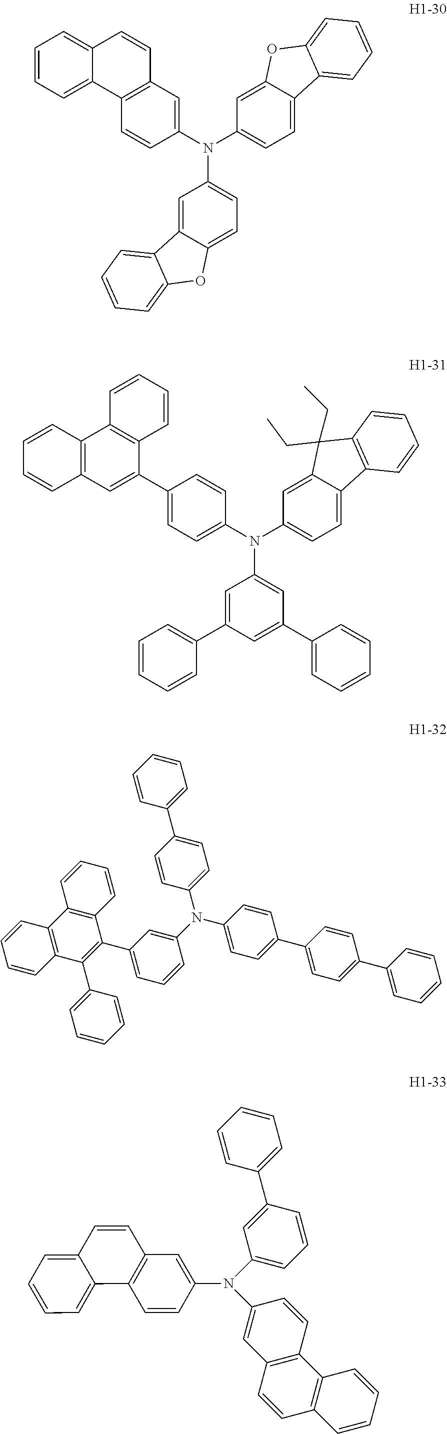 [SMILES: c1ccc2c(c1)ccc1cc(N(c3ccc4c(c3)oc3ccccc34)c3ccc4oc5ccccc5c4c3)ccc12 ; CCC1(CC)c2ccccc2-c2ccc(N(c3ccc(-c4cc5ccccc5c5ccccc45)cc3)c3cc(-c4ccccc4)cc(-c4ccccc4)c3)cc21 ; c1ccc(-c2ccc(-c3ccc(N(c4ccc(-c5ccccc5)cc4)c4cccc(-c5c(-c6ccccc6)c6ccccc6c6ccccc56)c4)cc3)cc2)cc1 ; c1ccc(-c2cccc(N(c3ccc4c(ccc5ccccc54)c3)c3ccc4c(ccc5ccccc54)c3)c2)cc1]

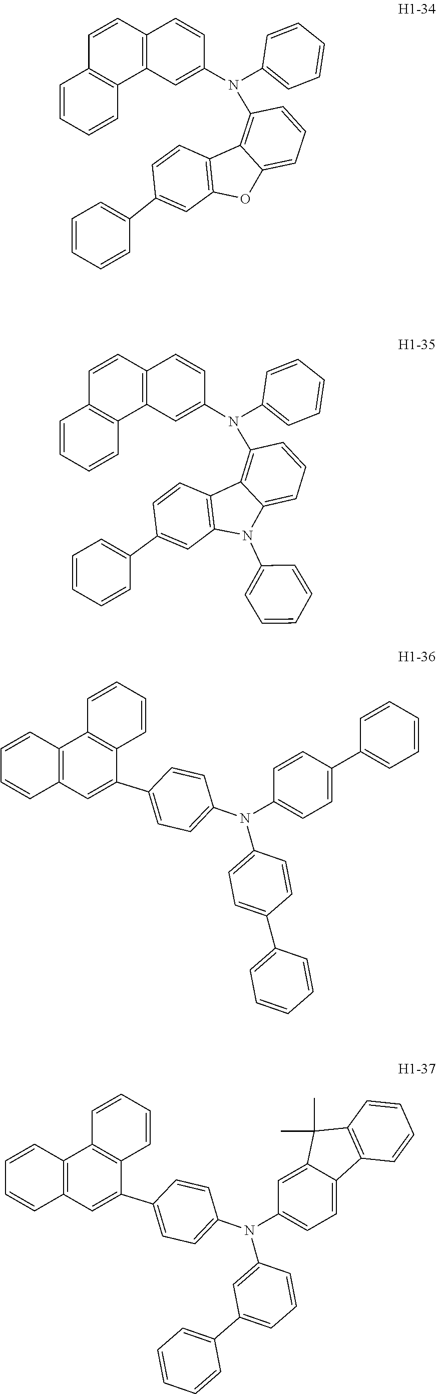

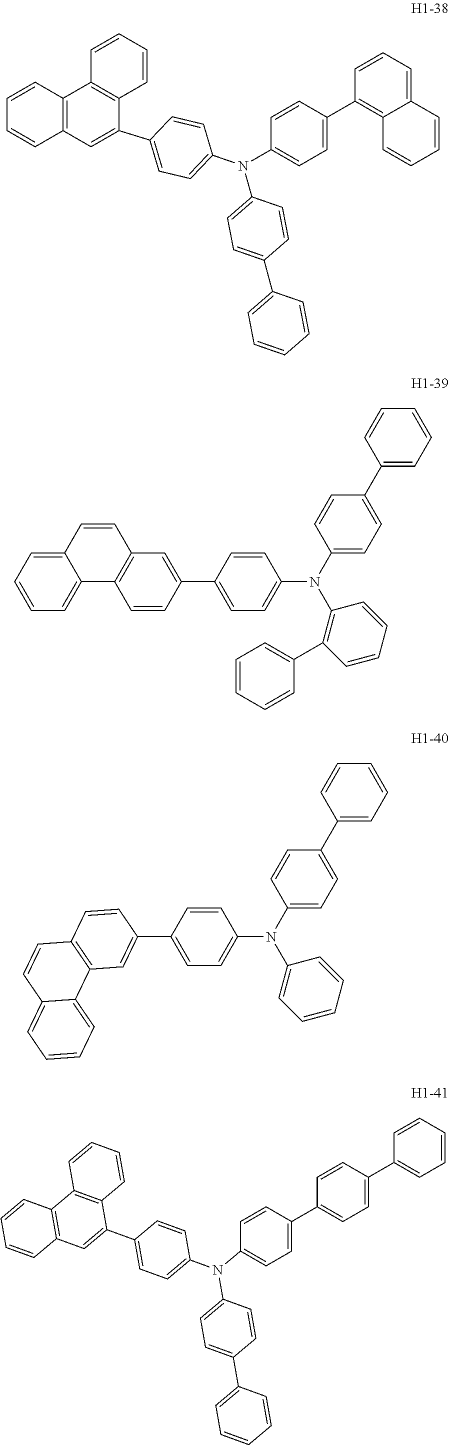 [SMILES: c1ccc(-c2ccc(N(c3ccc(-c4cccc5ccccc45)cc3)c3ccc(-c4cc5ccccc5c5ccccc45)cc3)cc2)cc1 ; c1ccc(-c2ccc(N(c3ccc(-c4ccc5c(ccc6ccccc65)c4)cc3)c3ccccc3-c3ccccc3)cc2)cc1 ; c1ccc(-c2ccc(N(c3ccccc3)c3ccc(-c4ccc5ccc6ccccc6c5c4)cc3)cc2)cc1 ; c1ccc(-c2ccc(-c3ccc(N(c4ccc(-c5ccccc5)cc4)c4ccc(-c5cc6ccccc6c6ccccc56)cc4)cc3)cc2)cc1]

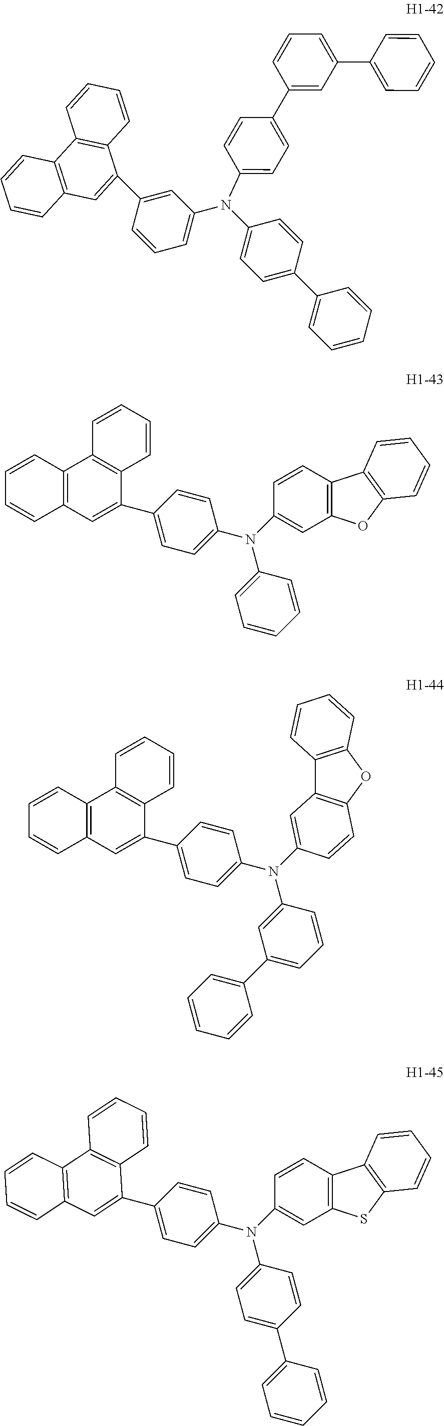 [SMILES: c1ccc(-c2ccc(N(c3ccc(-c4cccc(-c5ccccc5)c4)cc3)c3cccc(-c4cc5ccccc5c5ccccc45)c3)cc2)cc1 ; c1ccc(N(c2ccc(-c3cc4ccccc4c4ccccc34)cc2)c2ccc3c(c2)oc2ccccc23)cc1 ; c1ccc(-c2cccc(N(c3ccc(-c4cc5ccccc5c5ccccc45)cc3)c3ccc4oc5ccccc5c4c3)c2)cc1 ; c1ccc(-c2ccc(N(c3ccc(-c4cc5ccccc5c5ccccc45)cc3)c3ccc4c(c3)sc3ccccc34)cc2)cc1]

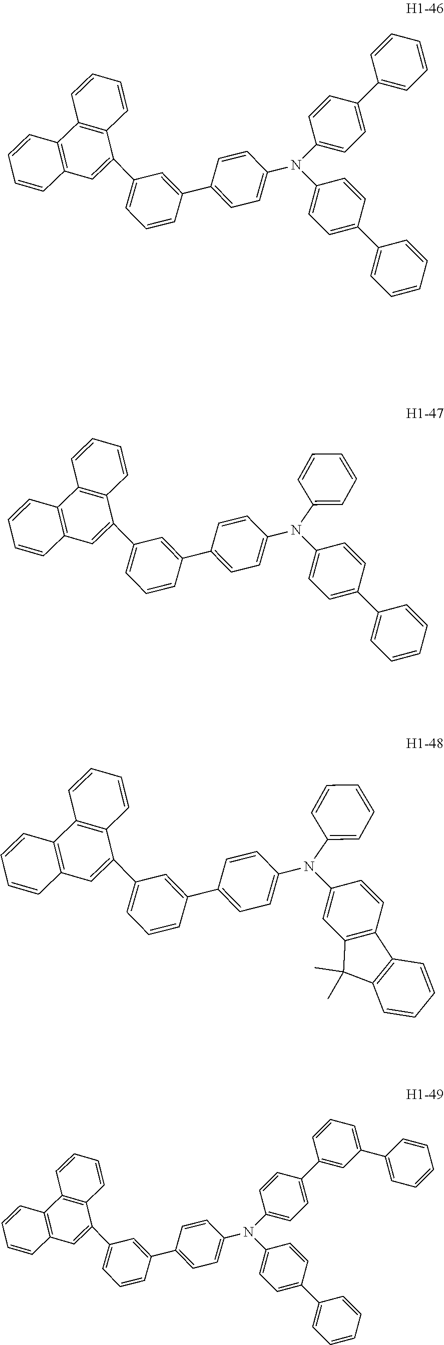 [SMILES: c1ccc(-c2ccc(N(c3ccc(-c4ccccc4)cc3)c3ccc(-c4cccc(-c5cc6ccccc6c6ccccc56)c4)cc3)cc2)cc1 ; c1ccc(-c2ccc(N(c3ccccc3)c3ccc(-c4cccc(-c5cc6ccccc6c6ccccc56)c4)cc3)cc2)cc1 ; CC1(C)c2ccccc2-c2ccc(N(c3ccccc3)c3ccc(-c4cccc(-c5cc6ccccc6c6ccccc56)c4)cc3)cc21 ; c1ccc(-c2ccc(N(c3ccc(-c4cccc(-c5ccccc5)c4)cc3)c3ccc(-c4cccc(-c5cc6ccccc6c6ccccc56)c4)cc3)cc2)cc1]

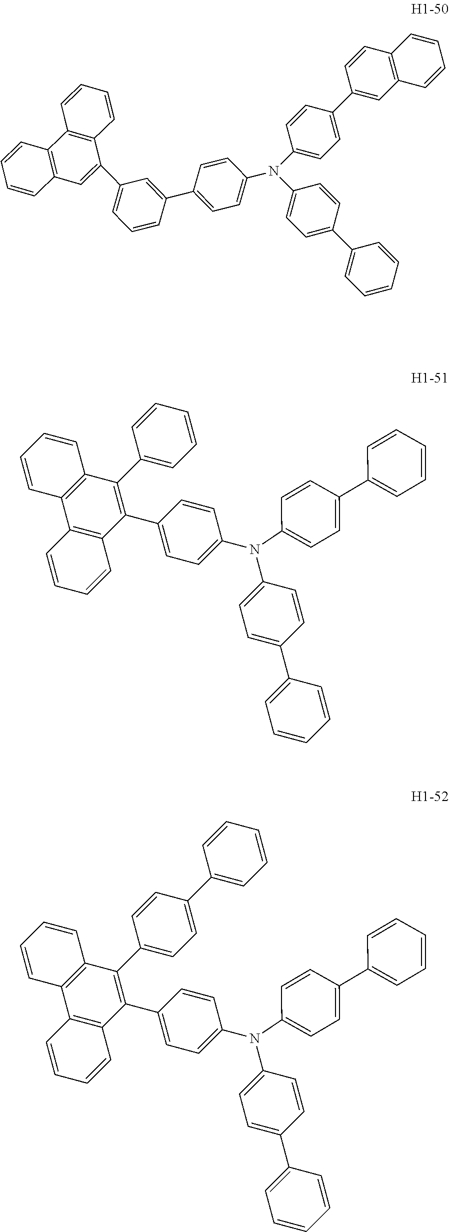 [SMILES: c1ccc(-c2ccc(N(c3ccc(-c4cccc(-c5cc6ccccc6c6ccccc56)c4)cc3)c3ccc(-c4ccc5ccccc5c4)cc3)cc2)cc1 ; c1ccc(-c2ccc(N(c3ccc(-c4ccccc4)cc3)c3ccc(-c4c(-c5ccccc5)c5ccccc5c5ccccc45)cc3)cc2)cc1 ; c1ccc(-c2ccc(-c3c(-c4ccc(N(c5ccc(-c6ccccc6)cc5)c5ccc(-c6ccccc6)cc5)cc4)c4ccccc4c4ccccc34)cc2)cc1]

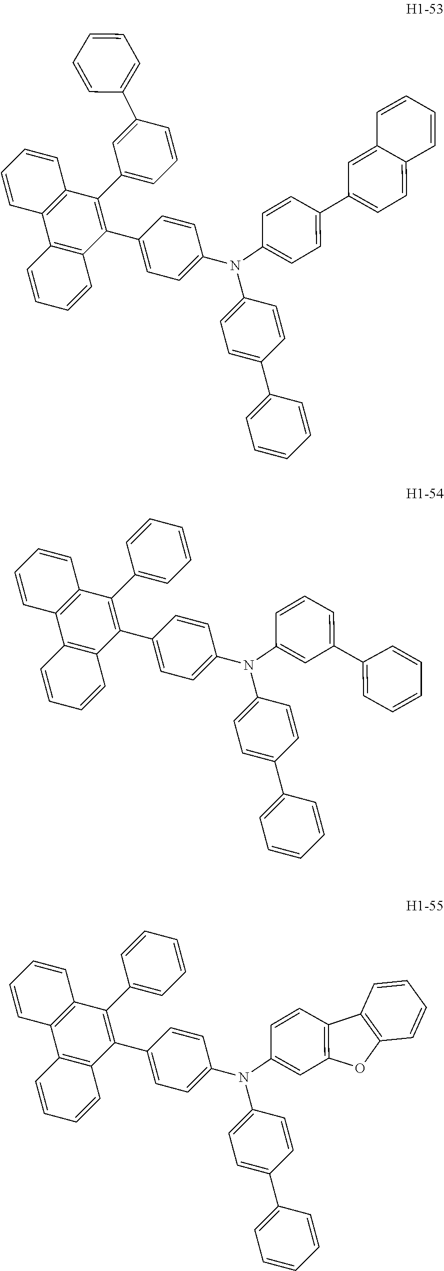 [SMILES: c1ccc(-c2ccc(N(c3ccc(-c4ccc5ccccc5c4)cc3)c3ccc(-c4c(-c5cccc(-c6ccccc6)c5)c5ccccc5c5ccccc45)cc3)cc2)cc1 ; c1ccc(-c2ccc(N(c3ccc(-c4c(-c5ccccc5)c5ccccc5c5ccccc45)cc3)c3cccc(-c4ccccc4)c3)cc2)cc1 ; c1ccc(-c2ccc(N(c3ccc(-c4c(-c5ccccc5)c5ccccc5c5ccccc45)cc3)c3ccc4c(c3)oc3ccccc34)cc2)cc1]

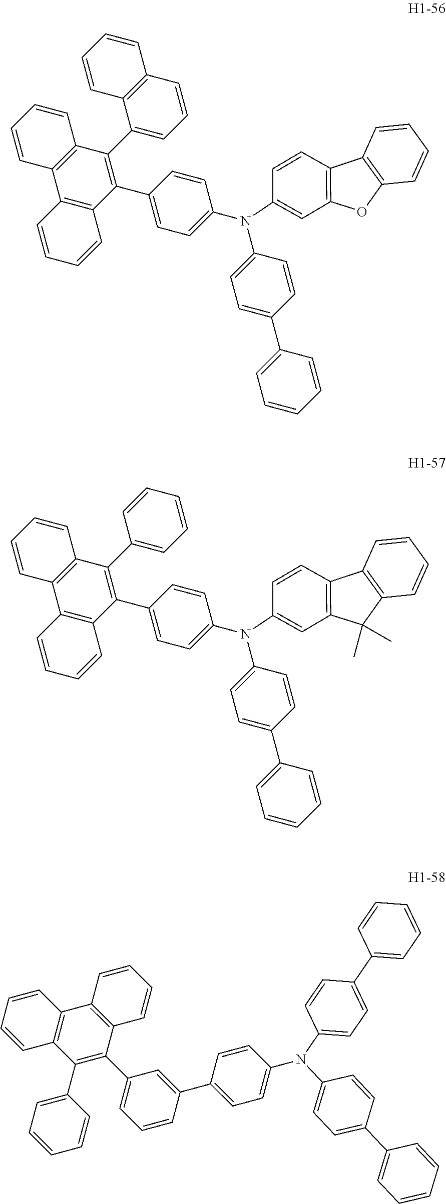 [SMILES: c1ccc(-c2ccc(N(c3ccc(-c4c(-c5cccc6ccccc56)c5ccccc5c5ccccc45)cc3)c3ccc4c(c3)oc3ccccc34)cc2)cc1 ; CC1(C)c2ccccc2-c2ccc(N(c3ccc(-c4ccccc4)cc3)c3ccc(-c4c(-c5ccccc5)c5ccccc5c5ccccc45)cc3)cc21 ; c1ccc(-c2ccc(N(c3ccc(-c4ccccc4)cc3)c3ccc(-c4cccc(-c5c(-c6ccccc6)c6ccccc6c6ccccc56)c4)cc3)cc2)cc1]

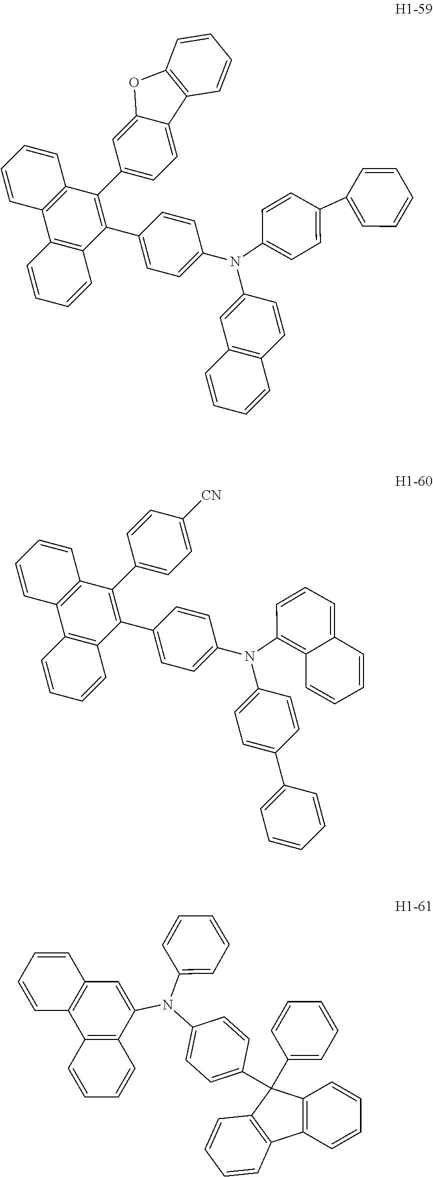 [SMILES: c1ccc(-c2ccc(N(c3ccc(-c4c(-c5ccc6c(c5)oc5ccccc56)c5ccccc5c5ccccc45)cc3)c3ccc4ccccc4c3)cc2)cc1 ; N#Cc1ccc(-c2c(-c3ccc(N(c4ccc(-c5ccccc5)cc4)c4cccc5ccccc45)cc3)c3ccccc3c3ccccc23)cc1 ; c1ccc(N(c2ccc(C3(c4ccccc4)c4ccccc4-c4ccccc43)cc2)c2cc3ccccc3c3ccccc23)cc1]

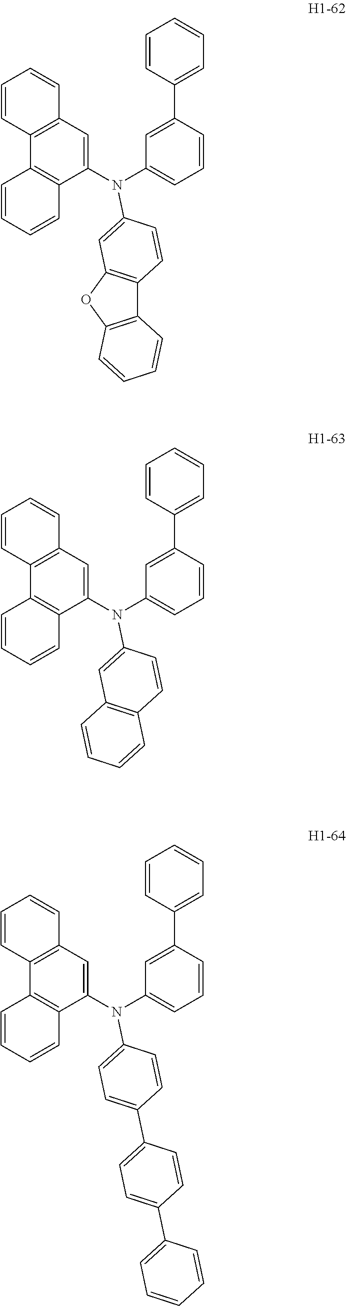 [SMILES: c1ccc(-c2cccc(N(c3ccc4c(c3)oc3ccccc34)c3cc4ccccc4c4ccccc34)c2)cc1 ; c1ccc(-c2cccc(N(c3ccc4ccccc4c3)c3cc4ccccc4c4ccccc34)c2)cc1 ; c1ccc(-c2ccc(-c3ccc(N(c4cccc(-c5ccccc5)c4)c4cc5ccccc5c5ccccc45)cc3)cc2)cc1]

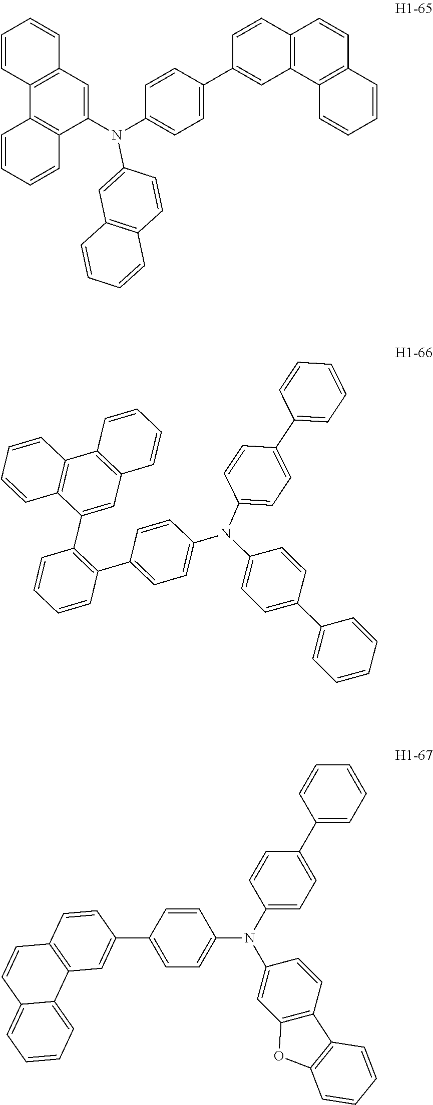 [SMILES: c1ccc2cc(N(c3ccc(-c4ccc5ccc6ccccc6c5c4)cc3)c3cc4ccccc4c4ccccc34)ccc2c1 ; c1ccc(-c2ccc(N(c3ccc(-c4ccccc4)cc3)c3ccc(-c4ccccc4-c4cc5ccccc5c5ccccc45)cc3)cc2)cc1 ; c1ccc(-c2ccc(N(c3ccc(-c4ccc5ccc6ccccc6c5c4)cc3)c3ccc4c(c3)oc3ccccc34)cc2)cc1]

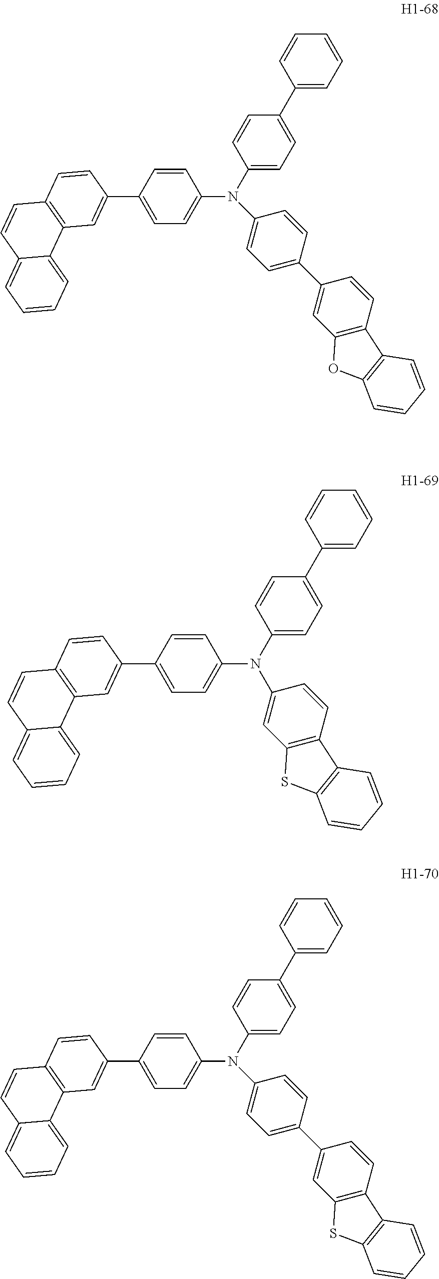 [SMILES: c1ccc(-c2ccc(N(c3ccc(-c4ccc5c(c4)oc4ccccc45)cc3)c3ccc(-c4ccc5ccc6ccccc6c5c4)cc3)cc2)cc1 ; c1ccc(-c2ccc(N(c3ccc(-c4ccc5ccc6ccccc6c5c4)cc3)c3ccc4c(c3)sc3ccccc34)cc2)cc1 ; c1ccc(-c2ccc(N(c3ccc(-c4ccc5c(c4)sc4ccccc45)cc3)c3ccc(-c4ccc5ccc6ccccc6c5c4)cc3)cc2)cc1]

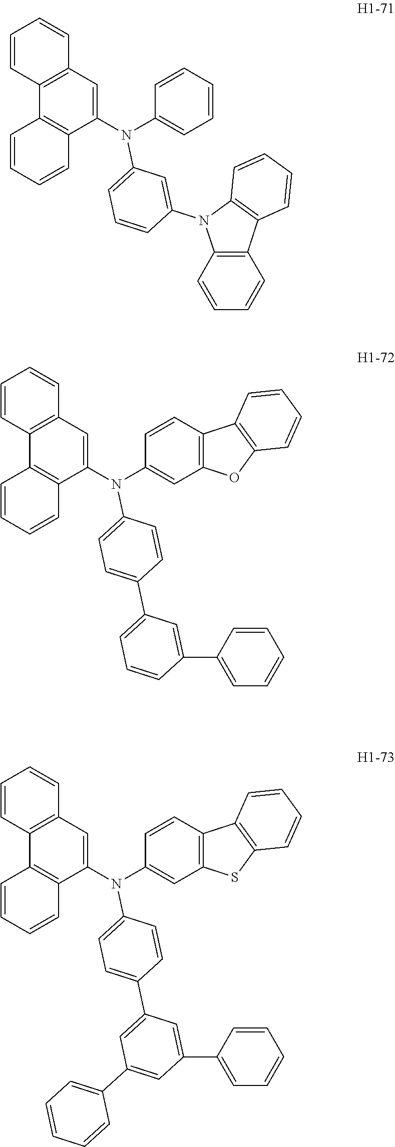 [SMILES: c1ccc(N(c2cccc(-n3c4ccccc4c4ccccc43)c2)c2cc3ccccc3c3ccccc23)cc1 ; c1ccc(-c2cccc(-c3ccc(N(c4ccc5c(c4)oc4ccccc45)c4cc5ccccc5c5ccccc45)cc3)c2)cc1 ; c1ccc(-c2cc(-c3ccccc3)cc(-c3ccc(N(c4ccc5c(c4)sc4ccccc45)c4cc5ccccc5c5ccccc45)cc3)c2)cc1]

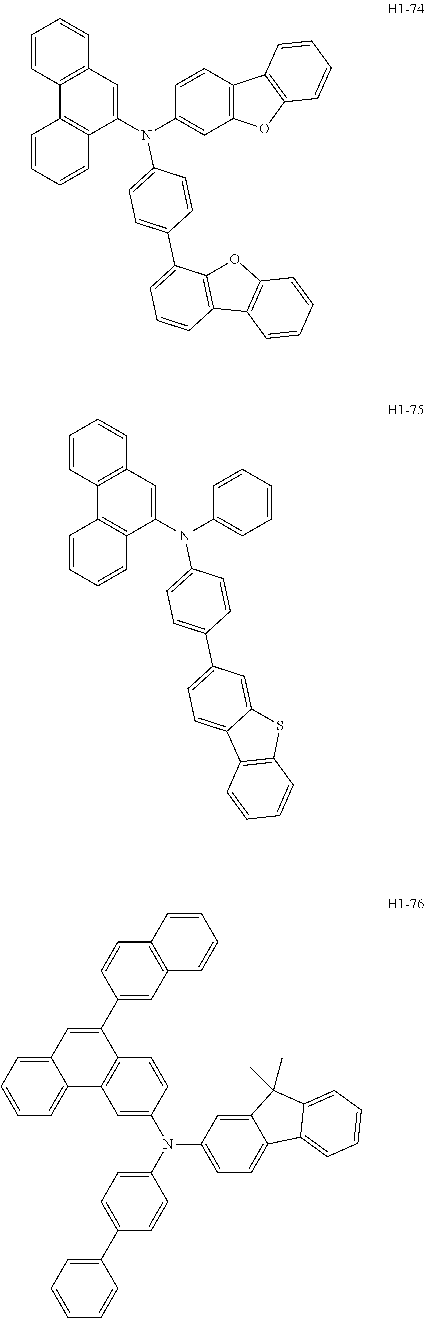 [SMILES: c1ccc2c(c1)cc(N(c1ccc(-c3cccc4c3oc3ccccc34)cc1)c1ccc3c(c1)oc1ccccc13)c1ccccc12 ; c1ccc(N(c2ccc(-c3ccc4c(c3)sc3ccccc34)cc2)c2cc3ccccc3c3ccccc23)cc1 ; CC1(C)c2ccccc2-c2ccc(N(c3ccc(-c4ccccc4)cc3)c3ccc4c(-c5ccc6ccccc6c5)cc5ccccc5c4c3)cc21]

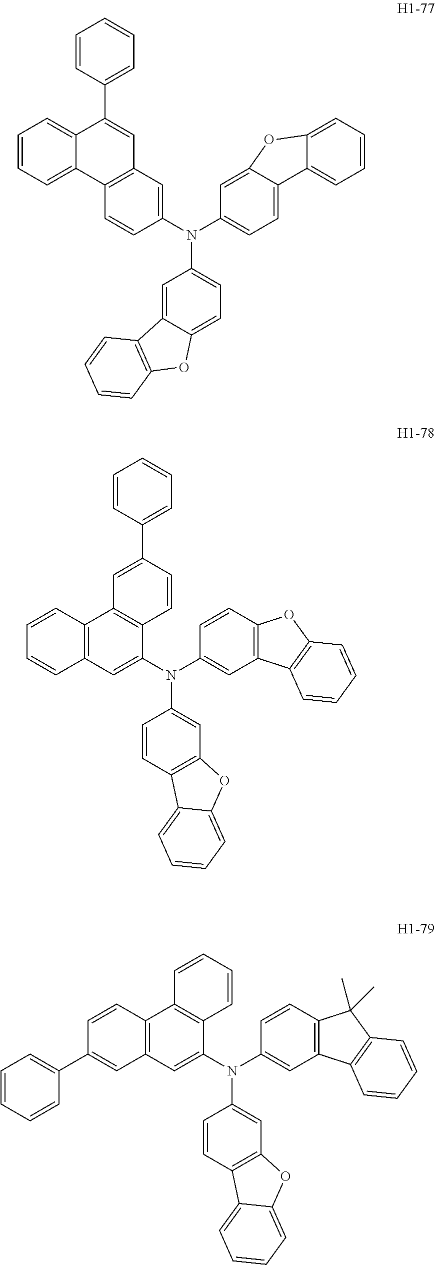 [SMILES: c1ccc(-c2cc3cc(N(c4ccc5c(c4)oc4ccccc45)c4ccc5oc6ccccc6c5c4)ccc3c3ccccc23)cc1 ; c1ccc(-c2ccc3c(N(c4ccc5c(c4)oc4ccccc45)c4ccc5oc6ccccc6c5c4)cc4ccccc4c3c2)cc1 ; CC1(C)c2ccccc2-c2cc(N(c3ccc4c(c3)oc3ccccc34)c3cc4cc(-c5ccccc5)ccc4c4ccccc34)ccc21]

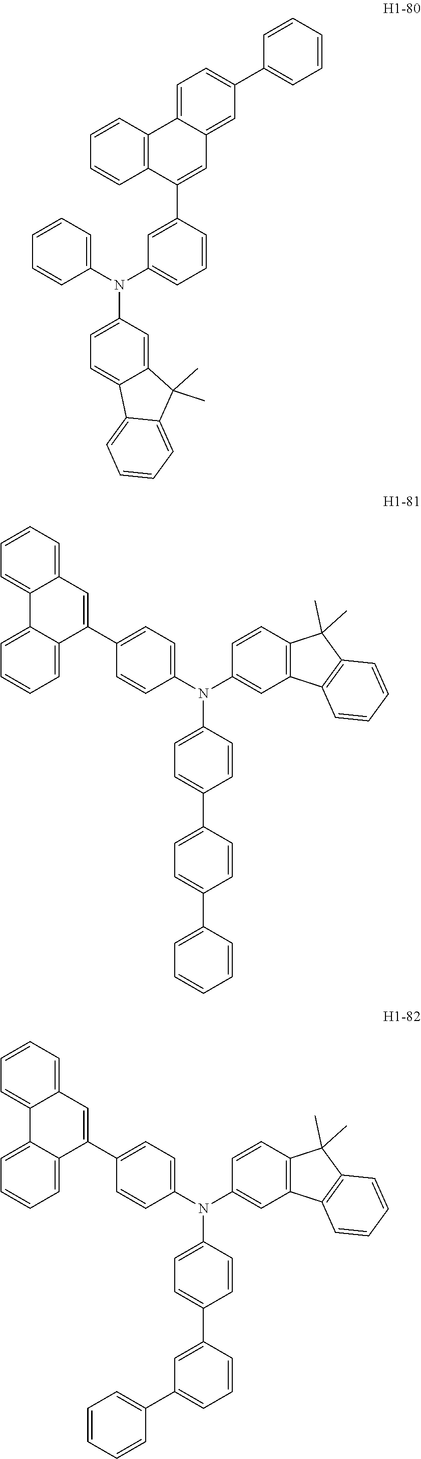 [SMILES: CC1(C)c2ccccc2-c2ccc(N(c3ccccc3)c3cccc(-c4cc5cc(-c6ccccc6)ccc5c5ccccc45)c3)cc21 ; CC1(C)c2ccccc2-c2cc(N(c3ccc(-c4ccc(-c5ccccc5)cc4)cc3)c3ccc(-c4cc5ccccc5c5ccccc45)cc3)ccc21 ; CC1(C)c2ccccc2-c2cc(N(c3ccc(-c4cccc(-c5ccccc5)c4)cc3)c3ccc(-c4cc5ccccc5c5ccccc45)cc3)ccc21]

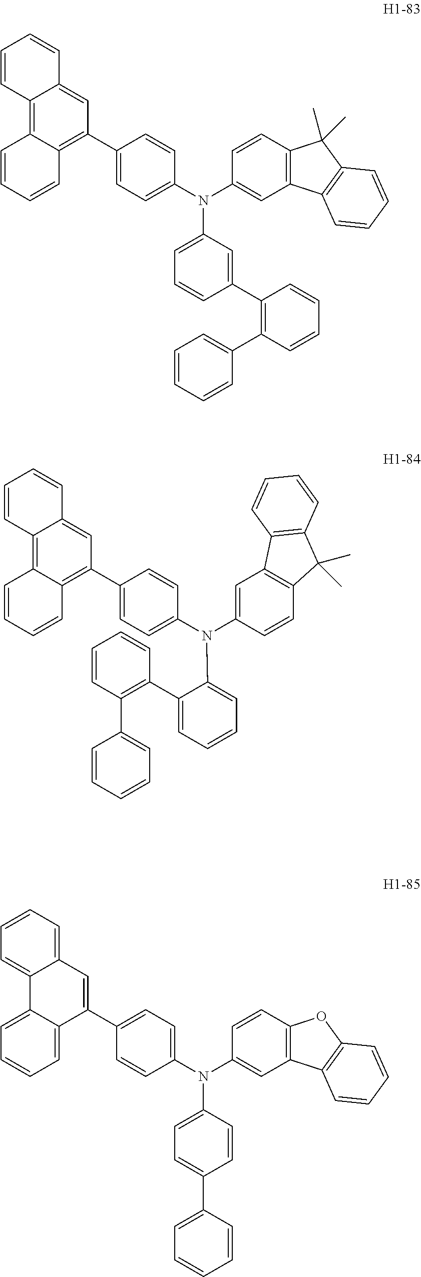 [SMILES: CC1(C)c2ccccc2-c2cc(N(c3ccc(-c4cc5ccccc5c5ccccc45)cc3)c3cccc(-c4ccccc4-c4ccccc4)c3)ccc21 ; CC1(C)c2ccccc2-c2cc(N(c3ccc(-c4cc5ccccc5c5ccccc45)cc3)c3ccccc3-c3ccccc3-c3ccccc3)ccc21 ; c1ccc(-c2ccc(N(c3ccc(-c4cc5ccccc5c5ccccc45)cc3)c3ccc4oc5ccccc5c4c3)cc2)cc1]

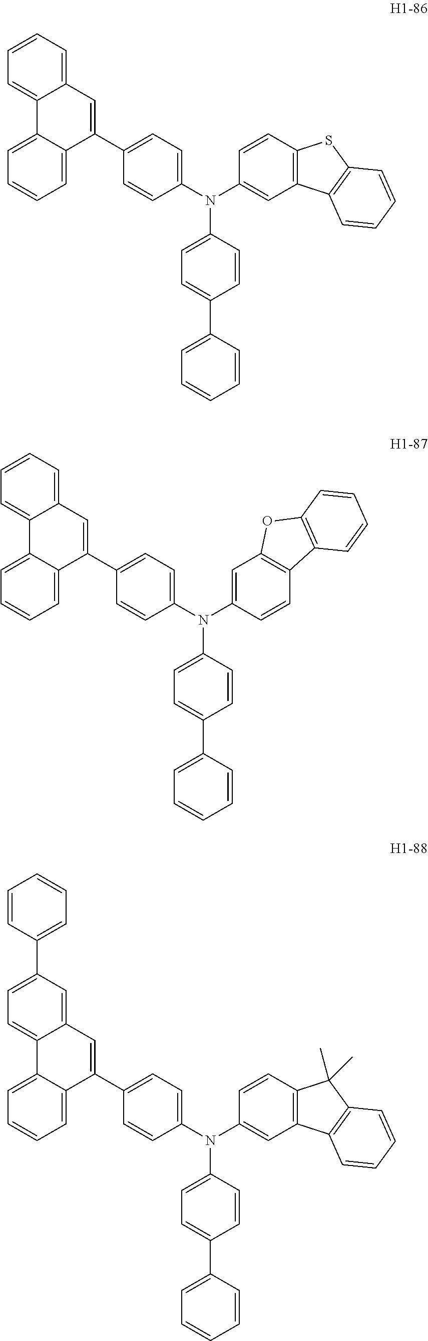 [SMILES: c1ccc(-c2ccc(N(c3ccc(-c4cc5ccccc5c5ccccc45)cc3)c3ccc4sc5ccccc5c4c3)cc2)cc1 ; c1ccc(-c2ccc(N(c3ccc(-c4cc5ccccc5c5ccccc45)cc3)c3ccc4c(c3)oc3ccccc34)cc2)cc1 ; CC1(C)c2ccccc2-c2cc(N(c3ccc(-c4ccccc4)cc3)c3ccc(-c4cc5cc(-c6ccccc6)ccc5c5ccccc45)cc3)ccc21]

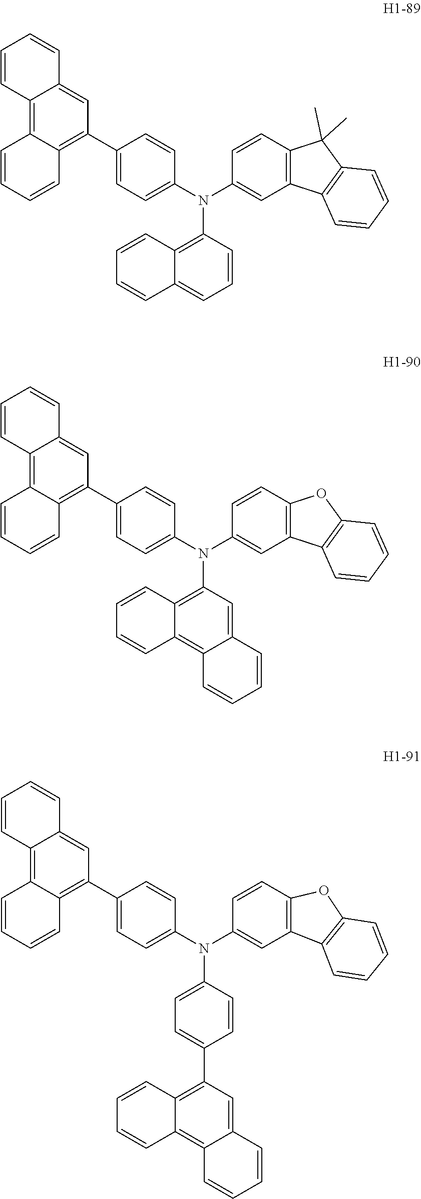

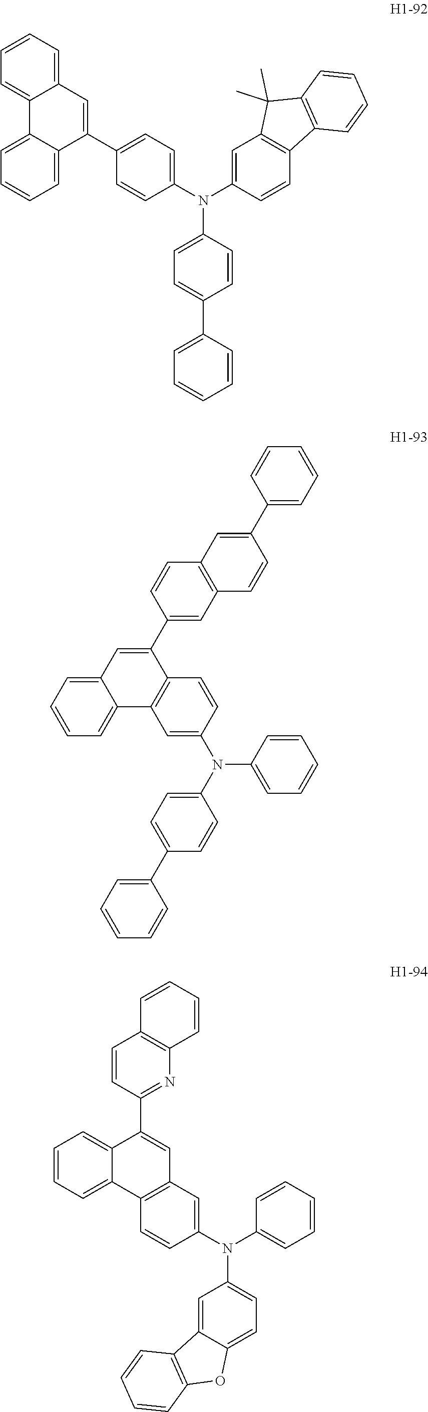 [SMILES: CC1(C)c2ccccc2-c2ccc(N(c3ccc(-c4ccccc4)cc3)c3ccc(-c4cc5ccccc5c5ccccc45)cc3)cc21 ; c1ccc(-c2ccc(N(c3ccccc3)c3ccc4c(-c5ccc6cc(-c7ccccc7)ccc6c5)cc5ccccc5c4c3)cc2)cc1 ; c1ccc(N(c2ccc3c(c2)cc(-c2ccc4ccccc4n2)c2ccccc23)c2ccc3oc4ccccc4c3c2)cc1]

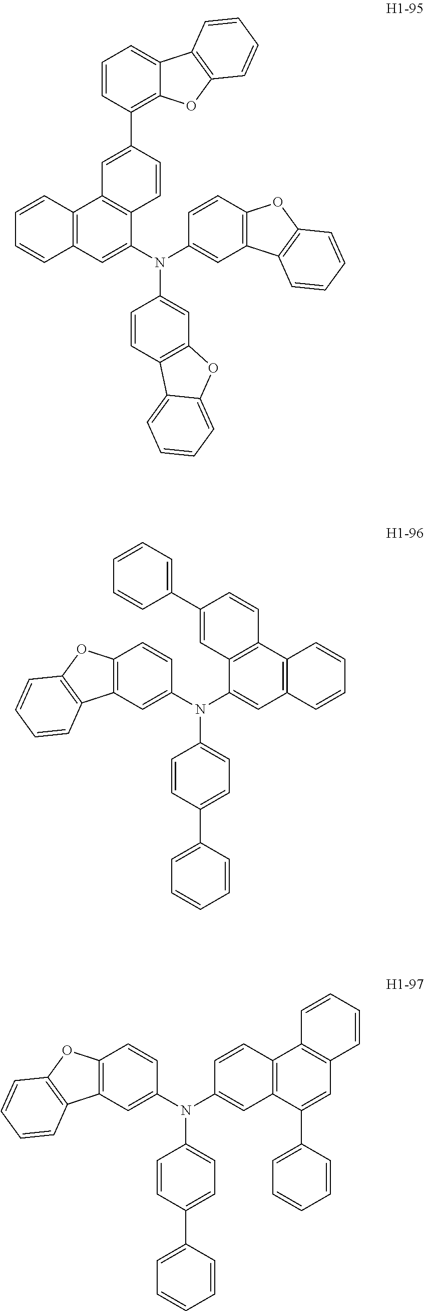 [SMILES: c1ccc2c(c1)cc(N(c1ccc3c(c1)oc1ccccc13)c1ccc3oc4ccccc4c3c1)c1ccc(-c3cccc4c3oc3ccccc34)cc12 ; c1ccc(-c2ccc(N(c3ccc4oc5ccccc5c4c3)c3cc4ccccc4c4ccc(-c5ccccc5)cc34)cc2)cc1 ; c1ccc(-c2ccc(N(c3ccc4c(c3)c(-c3ccccc3)cc3ccccc34)c3ccc4oc5ccccc5c4c3)cc2)cc1]

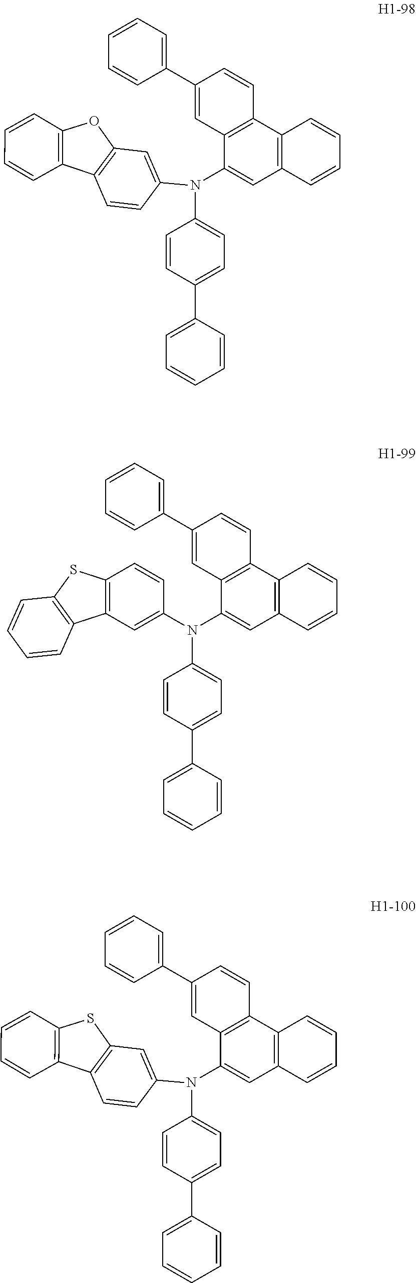 [SMILES: c1ccc(-c2ccc(N(c3ccc4c(c3)oc3ccccc34)c3cc4ccccc4c4ccc(-c5ccccc5)cc34)cc2)cc1 ; c1ccc(-c2ccc(N(c3ccc4sc5ccccc5c4c3)c3cc4ccccc4c4ccc(-c5ccccc5)cc34)cc2)cc1 ; c1ccc(-c2ccc(N(c3ccc4c(c3)sc3ccccc34)c3cc4ccccc4c4ccc(-c5ccccc5)cc34)cc2)cc1]

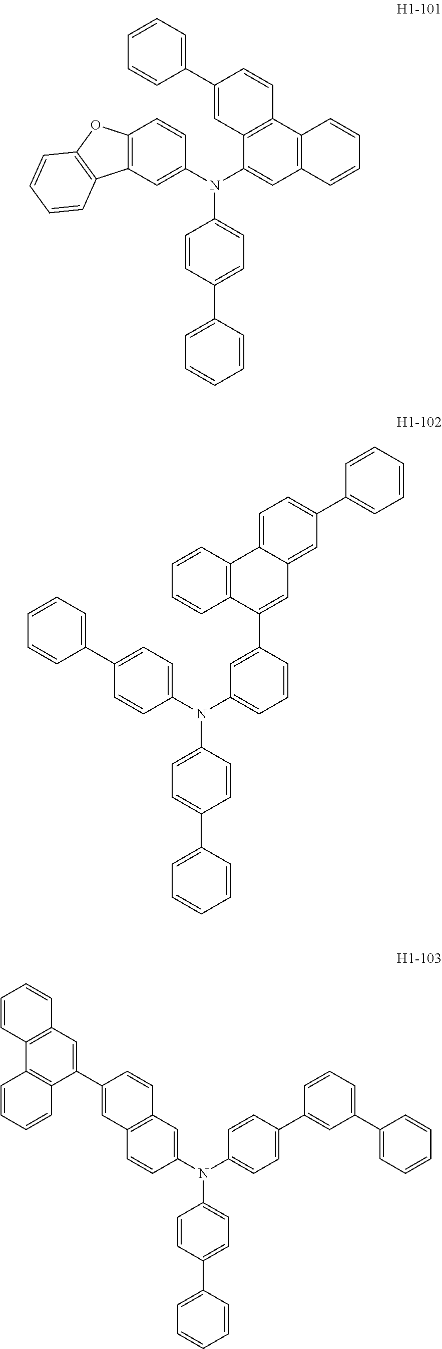 [SMILES: c1ccc(-c2ccc(N(c3ccc4oc5ccccc5c4c3)c3cc4ccccc4c4ccc(-c5ccccc5)cc34)cc2)cc1 ; c1ccc(-c2ccc(N(c3ccc(-c4ccccc4)cc3)c3cccc(-c4cc5cc(-c6ccccc6)ccc5c5ccccc45)c3)cc2)cc1 ; c1ccc(-c2ccc(N(c3ccc(-c4cccc(-c5ccccc5)c4)cc3)c3ccc4cc(-c5cc6ccccc6c6ccccc56)ccc4c3)cc2)cc1]

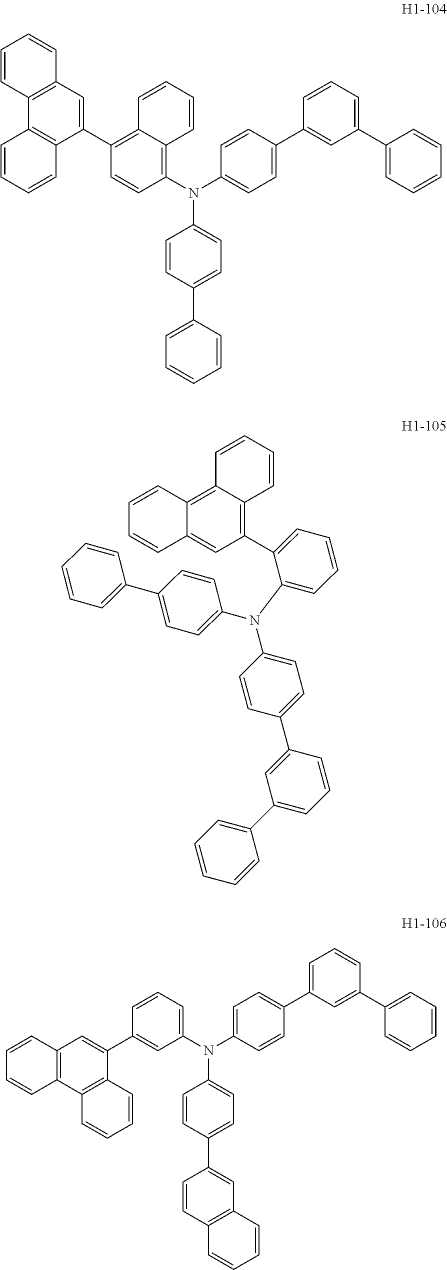 [SMILES: c1ccc(-c2ccc(N(c3ccc(-c4cccc(-c5ccccc5)c4)cc3)c3ccc(-c4cc5ccccc5c5ccccc45)c4ccccc34)cc2)cc1 ; c1ccc(-c2ccc(N(c3ccc(-c4cccc(-c5ccccc5)c4)cc3)c3ccccc3-c3cc4ccccc4c4ccccc34)cc2)cc1 ; c1ccc(-c2cccc(-c3ccc(N(c4ccc(-c5ccc6ccccc6c5)cc4)c4cccc(-c5cc6ccccc6c6ccccc56)c4)cc3)c2)cc1]

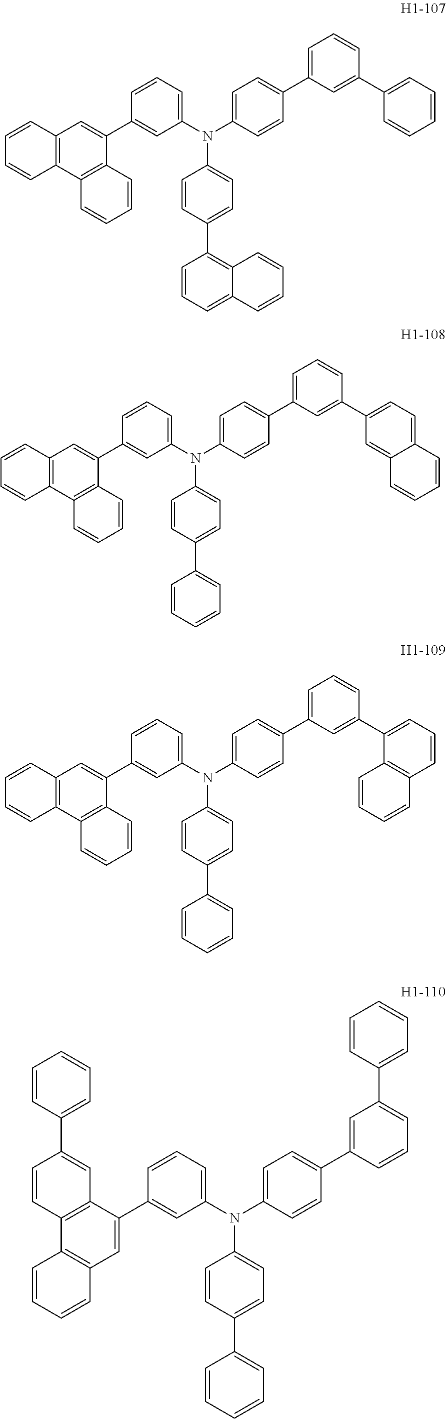 [SMILES: c1ccc(-c2cccc(-c3ccc(N(c4ccc(-c5cccc6ccccc56)cc4)c4cccc(-c5cc6ccccc6c6ccccc56)c4)cc3)c2)cc1 ; c1ccc(-c2ccc(N(c3ccc(-c4cccc(-c5ccc6ccccc6c5)c4)cc3)c3cccc(-c4cc5ccccc5c5ccccc45)c3)cc2)cc1 ; c1ccc(-c2ccc(N(c3ccc(-c4cccc(-c5cccc6ccccc56)c4)cc3)c3cccc(-c4cc5ccccc5c5ccccc45)c3)cc2)cc1 ; c1ccc(-c2ccc(N(c3ccc(-c4cccc(-c5ccccc5)c4)cc3)c3cccc(-c4cc5ccccc5c5ccc(-c6ccccc6)cc45)c3)cc2)cc1]

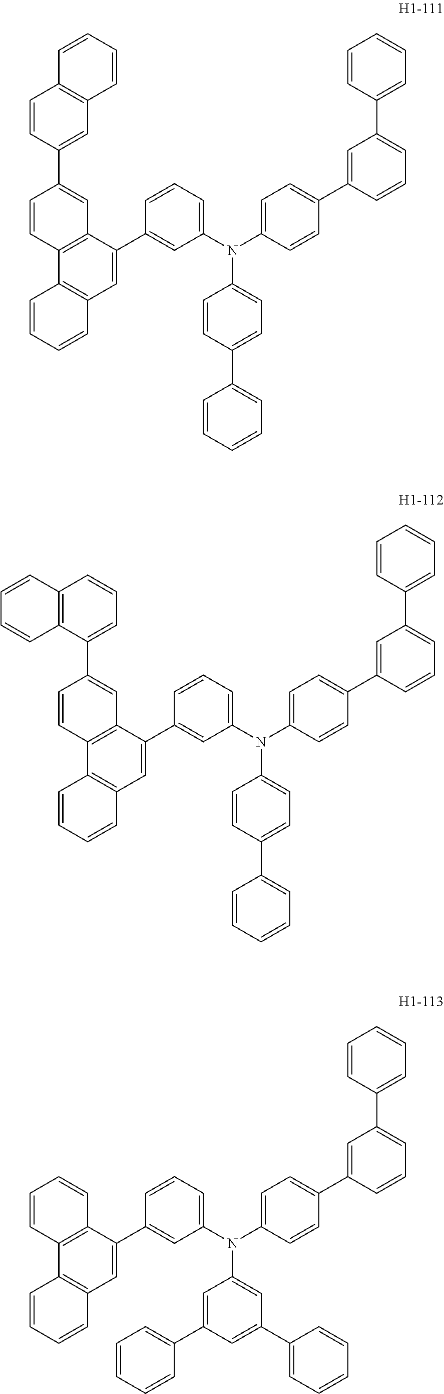 [SMILES: c1ccc(-c2ccc(N(c3ccc(-c4cccc(-c5ccccc5)c4)cc3)c3cccc(-c4cc5ccccc5c5ccc(-c6ccc7ccccc7c6)cc45)c3)cc2)cc1 ; c1ccc(-c2ccc(N(c3ccc(-c4cccc(-c5ccccc5)c4)cc3)c3cccc(-c4cc5ccccc5c5ccc(-c6cccc7ccccc67)cc45)c3)cc2)cc1 ; c1ccc(-c2cccc(-c3ccc(N(c4cc(-c5ccccc5)cc(-c5ccccc5)c4)c4cccc(-c5cc6ccccc6c6ccccc56)c4)cc3)c2)cc1]

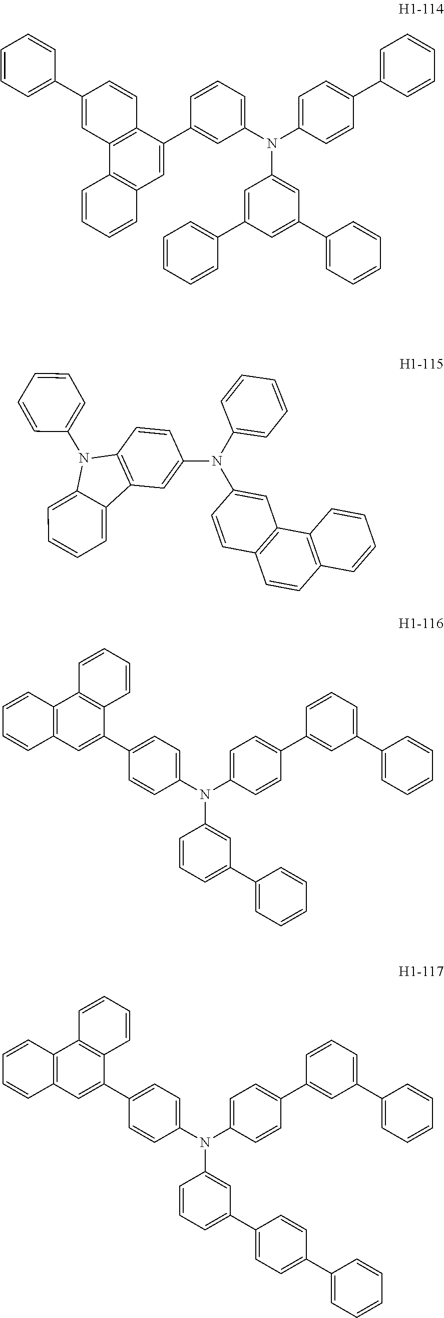 [SMILES: c1ccc(-c2ccc(N(c3cc(-c4ccccc4)cc(-c4ccccc4)c3)c3cccc(-c4cc5ccccc5c5cc(-c6ccccc6)ccc45)c3)cc2)cc1 ; c1ccc(N(c2ccc3ccc4ccccc4c3c2)c2ccc3c(c2)c2ccccc2n3-c2ccccc2)cc1 ; c1ccc(-c2cccc(-c3ccc(N(c4ccc(-c5cc6ccccc6c6ccccc56)cc4)c4cccc(-c5ccccc5)c4)cc3)c2)cc1 ; c1ccc(-c2ccc(-c3cccc(N(c4ccc(-c5cccc(-c6ccccc6)c5)cc4)c4ccc(-c5cc6ccccc6c6ccccc56)cc4)c3)cc2)cc1]

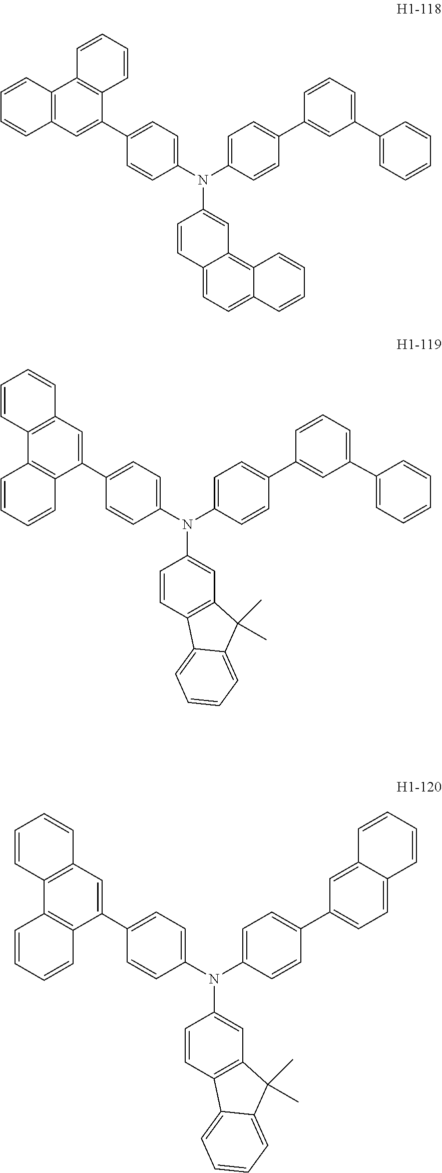 [SMILES: c1ccc(-c2cccc(-c3ccc(N(c4ccc(-c5cc6ccccc6c6ccccc56)cc4)c4ccc5ccc6ccccc6c5c4)cc3)c2)cc1 ; CC1(C)c2ccccc2-c2ccc(N(c3ccc(-c4cccc(-c5ccccc5)c4)cc3)c3ccc(-c4cc5ccccc5c5ccccc45)cc3)cc21 ; CC1(C)c2ccccc2-c2ccc(N(c3ccc(-c4ccc5ccccc5c4)cc3)c3ccc(-c4cc5ccccc5c5ccccc45)cc3)cc21]

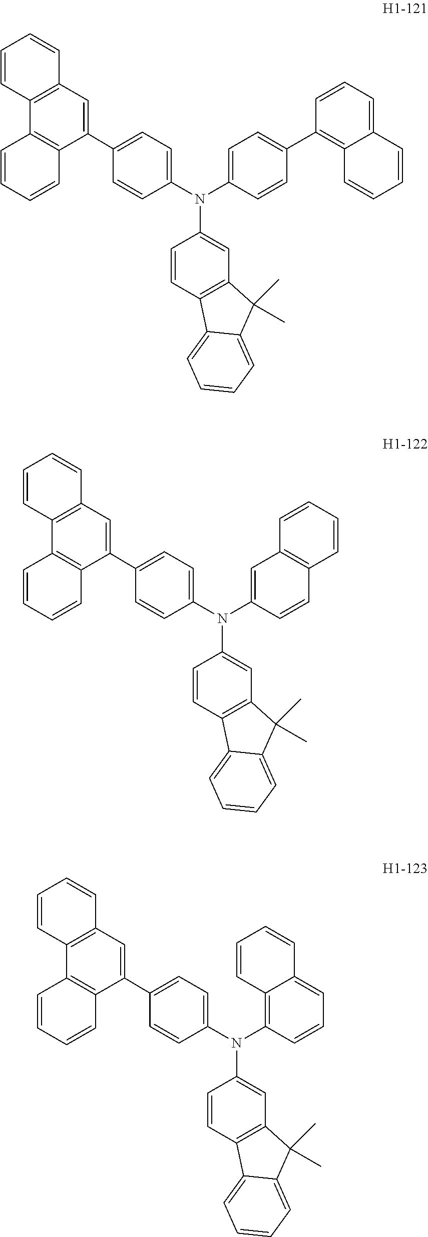 [SMILES: CC1(C)c2ccccc2-c2ccc(N(c3ccc(-c4cccc5ccccc45)cc3)c3ccc(-c4cc5ccccc5c5ccccc45)cc3)cc21 ; CC1(C)c2ccccc2-c2ccc(N(c3ccc(-c4cc5ccccc5c5ccccc45)cc3)c3ccc4ccccc4c3)cc21 ; CC1(C)c2ccccc2-c2ccc(N(c3ccc(-c4cc5ccccc5c5ccccc45)cc3)c3cccc4ccccc34)cc21]

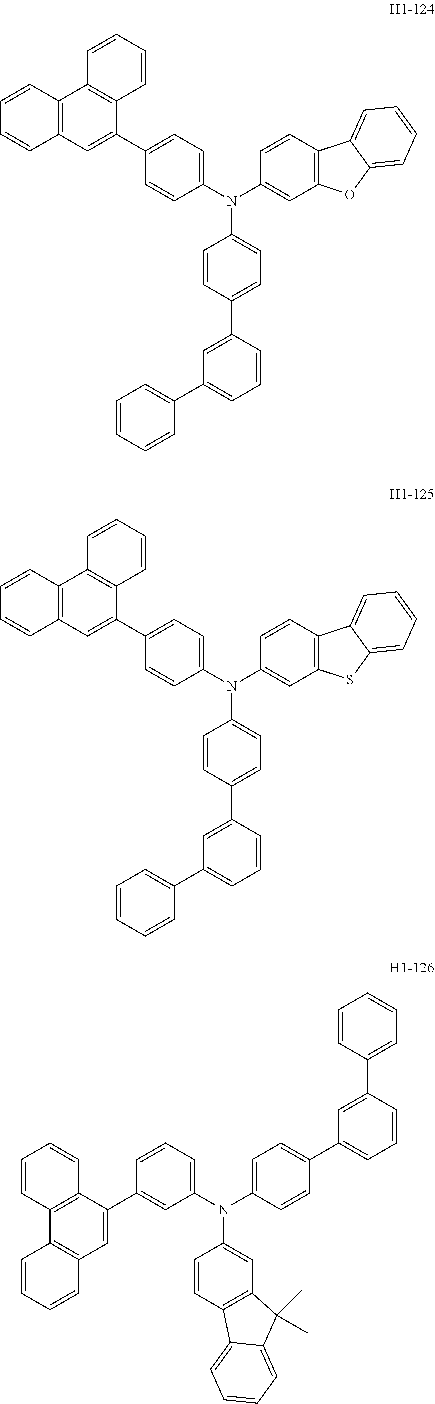 [SMILES: c1ccc(-c2cccc(-c3ccc(N(c4ccc(-c5cc6ccccc6c6ccccc56)cc4)c4ccc5c(c4)oc4ccccc45)cc3)c2)cc1 ; c1ccc(-c2cccc(-c3ccc(N(c4ccc(-c5cc6ccccc6c6ccccc56)cc4)c4ccc5c(c4)sc4ccccc45)cc3)c2)cc1 ; CC1(C)c2ccccc2-c2ccc(N(c3ccc(-c4cccc(-c5ccccc5)c4)cc3)c3cccc(-c4cc5ccccc5c5ccccc45)c3)cc21]

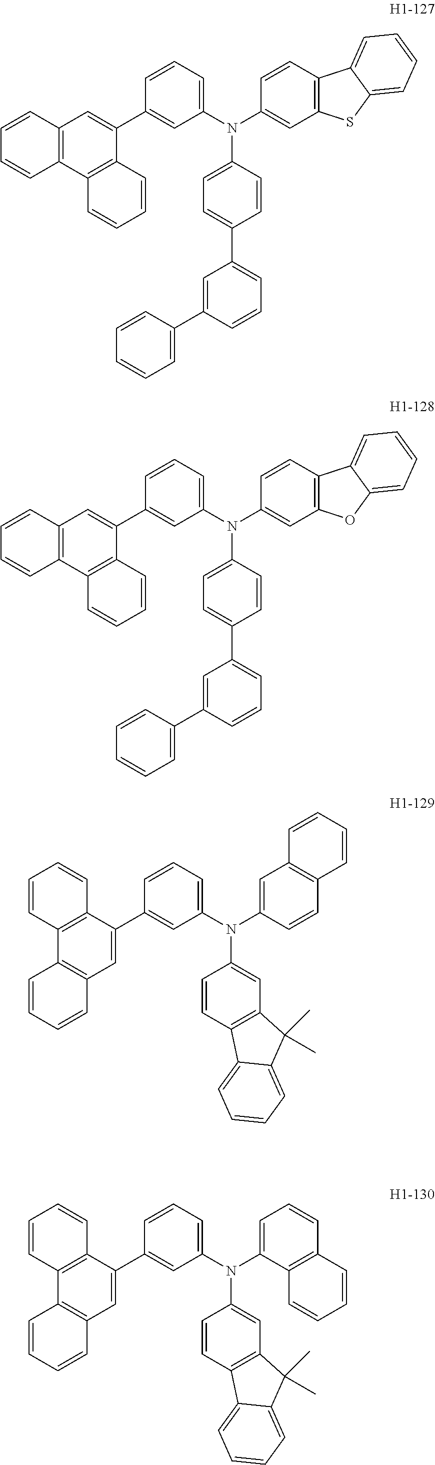 [SMILES: c1ccc(-c2cccc(-c3ccc(N(c4cccc(-c5cc6ccccc6c6ccccc56)c4)c4ccc5c(c4)sc4ccccc45)cc3)c2)cc1 ; c1ccc(-c2cccc(-c3ccc(N(c4cccc(-c5cc6ccccc6c6ccccc56)c4)c4ccc5c(c4)oc4ccccc45)cc3)c2)cc1 ; CC1(C)c2ccccc2-c2ccc(N(c3cccc(-c4cc5ccccc5c5ccccc45)c3)c3ccc4ccccc4c3)cc21 ; CC1(C)c2ccccc2-c2ccc(N(c3cccc(-c4cc5ccccc5c5ccccc45)c3)c3cccc4ccccc34)cc21]

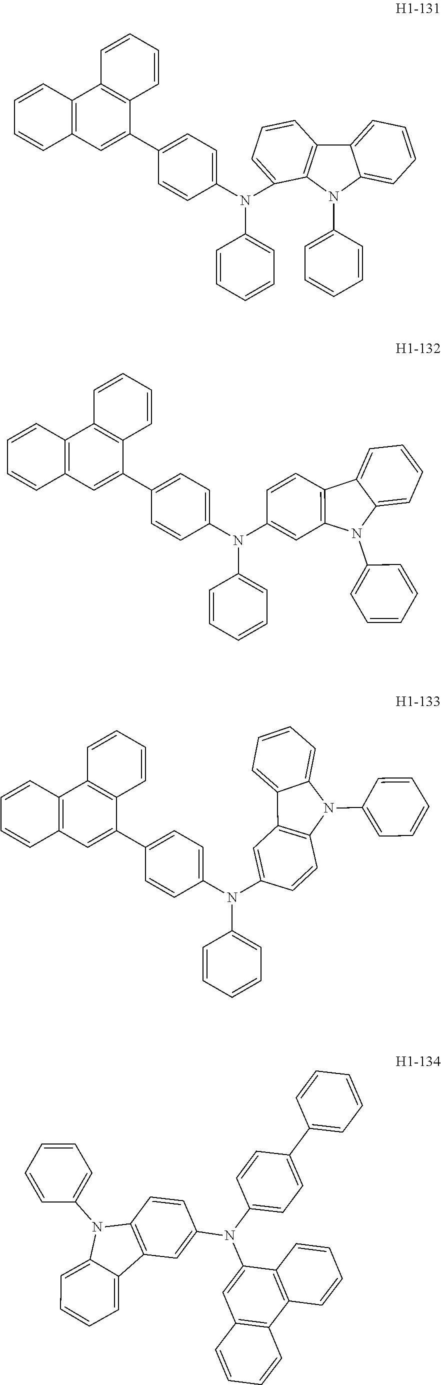 [SMILES: c1ccc(N(c2ccc(-c3cc4ccccc4c4ccccc34)cc2)c2cccc3c4ccccc4n(-c4ccccc4)c23)cc1 ; c1ccc(N(c2ccc(-c3cc4ccccc4c4ccccc34)cc2)c2ccc3c4ccccc4n(-c4ccccc4)c3c2)cc1 ; c1ccc(N(c2ccc(-c3cc4ccccc4c4ccccc34)cc2)c2ccc3c(c2)c2ccccc2n3-c2ccccc2)cc1 ; c1ccc(-c2ccc(N(c3ccc4c(c3)c3ccccc3n4-c3ccccc3)c3cc4ccccc4c4ccccc34)cc2)cc1]

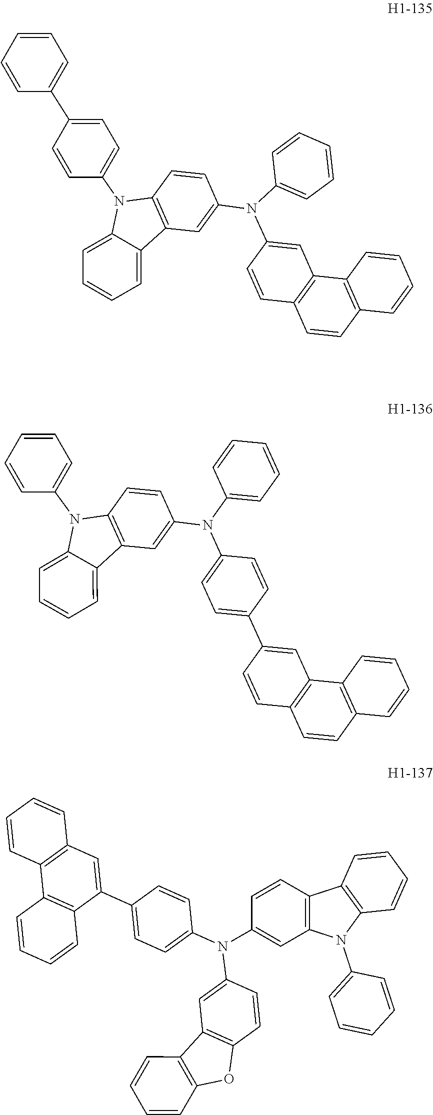 [SMILES: c1ccc(-c2ccc(-n3c4ccccc4c4cc(N(c5ccccc5)c5ccc6ccc7ccccc7c6c5)ccc43)cc2)cc1 ; c1ccc(N(c2ccc(-c3ccc4ccc5ccccc5c4c3)cc2)c2ccc3c(c2)c2ccccc2n3-c2ccccc2)cc1 ; c1ccc(-n2c3ccccc3c3ccc(N(c4ccc(-c5cc6ccccc6c6ccccc56)cc4)c4ccc5oc6ccccc6c5c4)cc32)cc1]

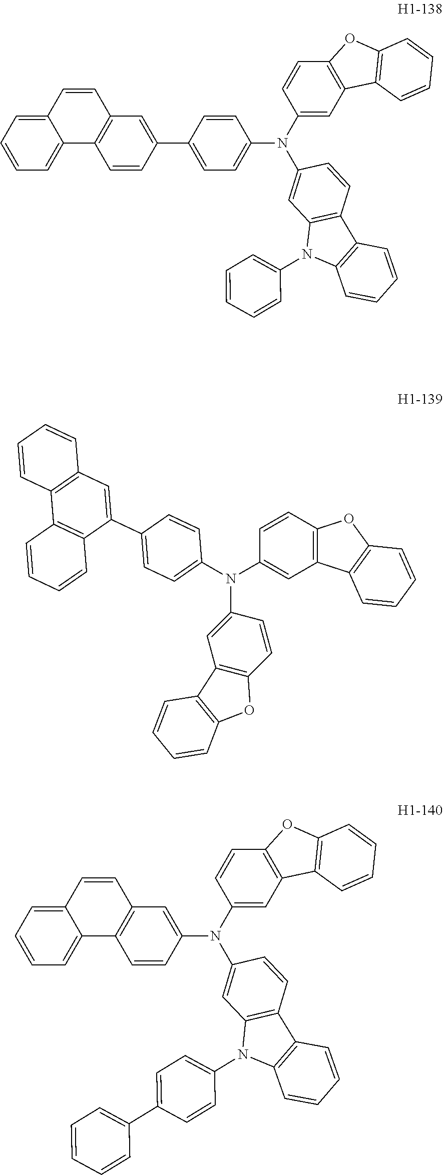 [SMILES: c1ccc(-n2c3ccccc3c3ccc(N(c4ccc(-c5ccc6c(ccc7ccccc76)c5)cc4)c4ccc5oc6ccccc6c5c4)cc32)cc1 ; c1ccc2c(c1)cc(-c1ccc(N(c3ccc4oc5ccccc5c4c3)c3ccc4oc5ccccc5c4c3)cc1)c1ccccc12 ; c1ccc(-c2ccc(-n3c4ccccc4c4ccc(N(c5ccc6c(ccc7ccccc76)c5)c5ccc6oc7ccccc7c6c5)cc43)cc2)cc1]

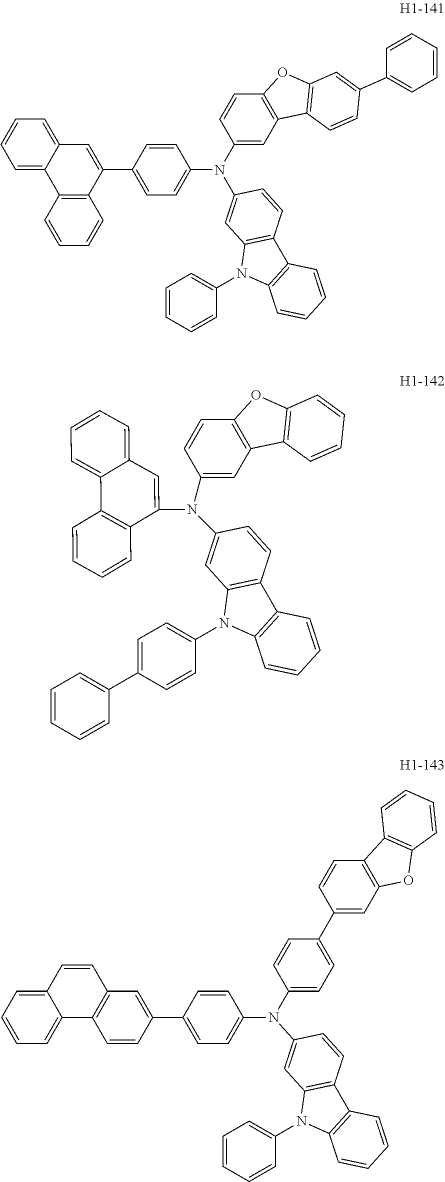 [SMILES: c1ccc(-c2ccc3c(c2)oc2ccc(N(c4ccc(-c5cc6ccccc6c6ccccc56)cc4)c4ccc5c6ccccc6n(-c6ccccc6)c5c4)cc23)cc1 ; c1ccc(-c2ccc(-n3c4ccccc4c4ccc(N(c5ccc6oc7ccccc7c6c5)c5cc6ccccc6c6ccccc56)cc43)cc2)cc1 ; c1ccc(-n2c3ccccc3c3ccc(N(c4ccc(-c5ccc6c(ccc7ccccc76)c5)cc4)c4ccc(-c5ccc6c(c5)oc5ccccc56)cc4)cc32)cc1]

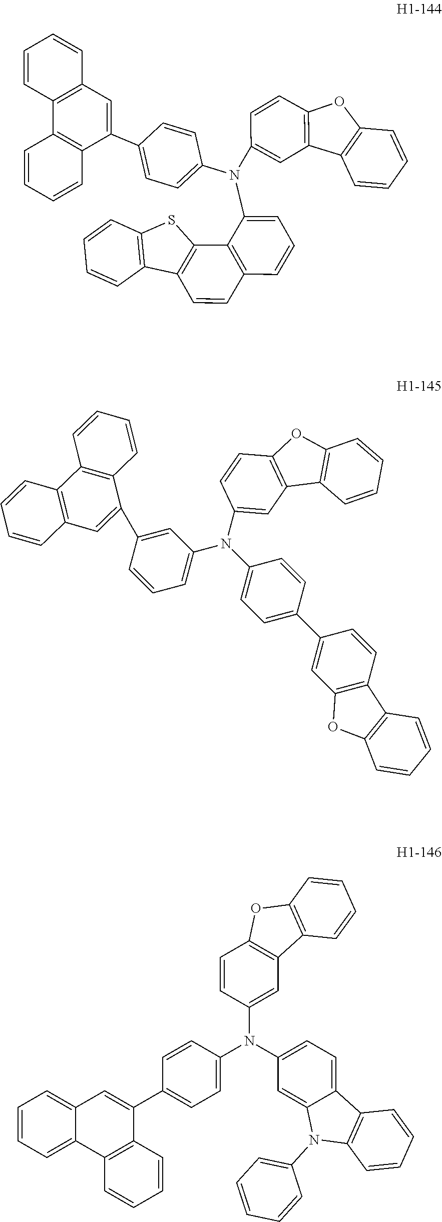 [SMILES: c1ccc2c(c1)cc(-c1ccc(N(c3ccc4oc5ccccc5c4c3)c3cccc4ccc5c6ccccc6sc5c34)cc1)c1ccccc12 ; c1cc(-c2cc3ccccc3c3ccccc23)cc(N(c2ccc(-c3ccc4c(c3)oc3ccccc34)cc2)c2ccc3oc4ccccc4c3c2)c1 ; c1ccc(-n2c3ccccc3c3ccc(N(c4ccc(-c5cc6ccccc6c6ccccc56)cc4)c4ccc5oc6ccccc6c5c4)cc32)cc1]

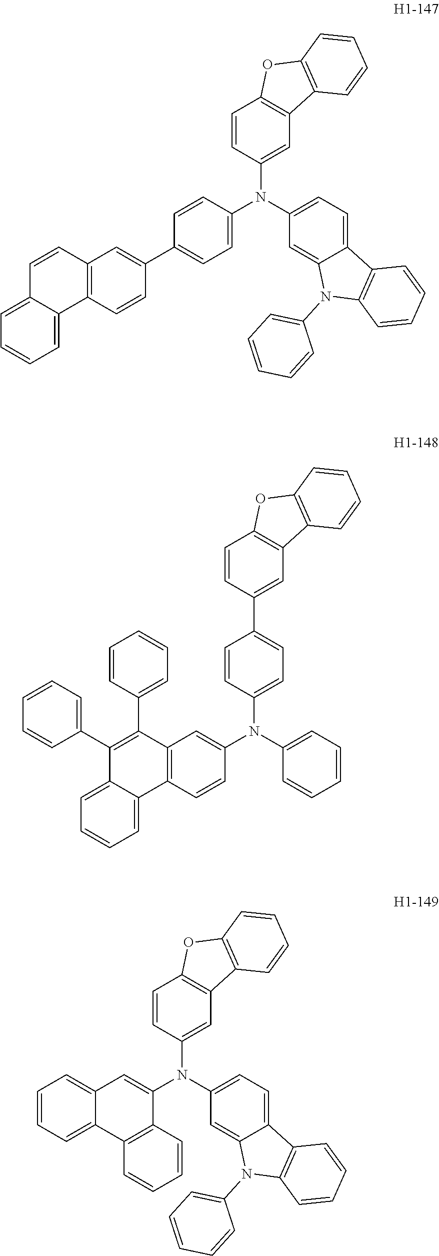 [SMILES: c1ccc(-n2c3ccccc3c3ccc(N(c4ccc(-c5ccc6c(ccc7ccccc76)c5)cc4)c4ccc5oc6ccccc6c5c4)cc32)cc1 ; c1ccc(-c2c(-c3ccccc3)c3cc(N(c4ccccc4)c4ccc(-c5ccc6oc7ccccc7c6c5)cc4)ccc3c3ccccc23)cc1 ; c1ccc(-n2c3ccccc3c3ccc(N(c4ccc5oc6ccccc6c5c4)c4cc5ccccc5c5ccccc45)cc32)cc1]

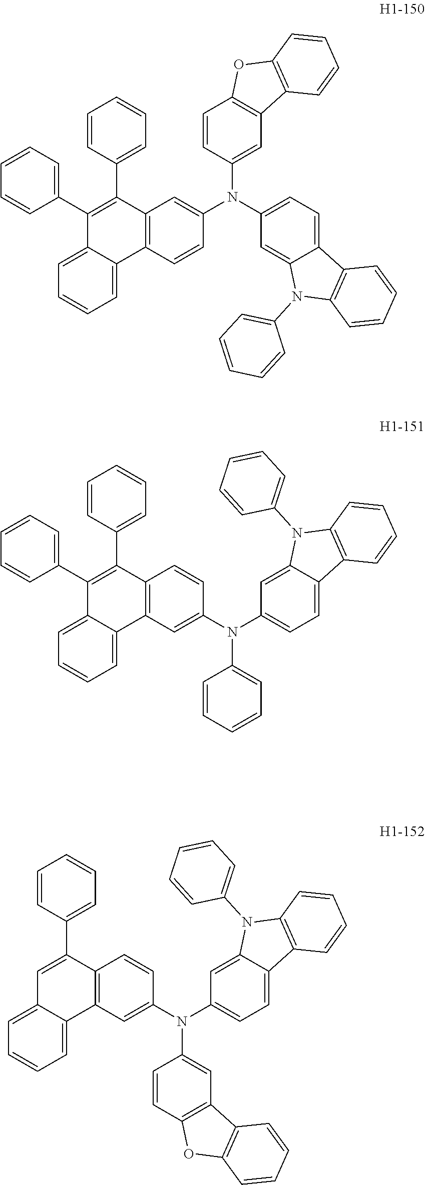 [SMILES: c1ccc(-c2c(-c3ccccc3)c3cc(N(c4ccc5oc6ccccc6c5c4)c4ccc5c6ccccc6n(-c6ccccc6)c5c4)ccc3c3ccccc23)cc1 ; c1ccc(-c2c(-c3ccccc3)c3ccc(N(c4ccccc4)c4ccc5c6ccccc6n(-c6ccccc6)c5c4)cc3c3ccccc23)cc1 ; c1ccc(-c2cc3ccccc3c3cc(N(c4ccc5oc6ccccc6c5c4)c4ccc5c6ccccc6n(-c6ccccc6)c5c4)ccc23)cc1]

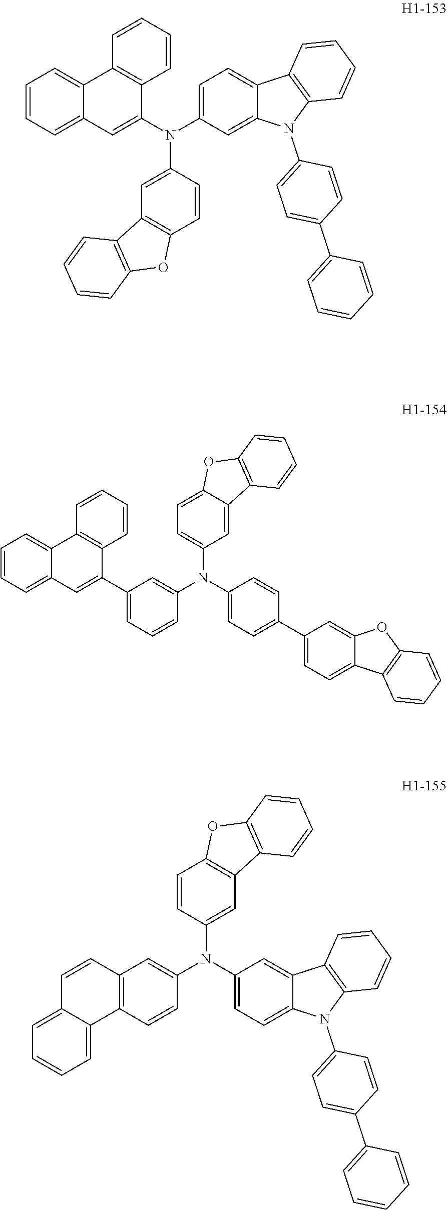 [SMILES: c1ccc(-c2ccc(-n3c4ccccc4c4ccc(N(c5ccc6oc7ccccc7c6c5)c5cc6ccccc6c6ccccc56)cc43)cc2)cc1 ; c1cc(-c2cc3ccccc3c3ccccc23)cc(N(c2ccc(-c3ccc4c(c3)oc3ccccc34)cc2)c2ccc3oc4ccccc4c3c2)c1 ; c1ccc(-c2ccc(-n3c4ccccc4c4cc(N(c5ccc6c(ccc7ccccc76)c5)c5ccc6oc7ccccc7c6c5)ccc43)cc2)cc1]

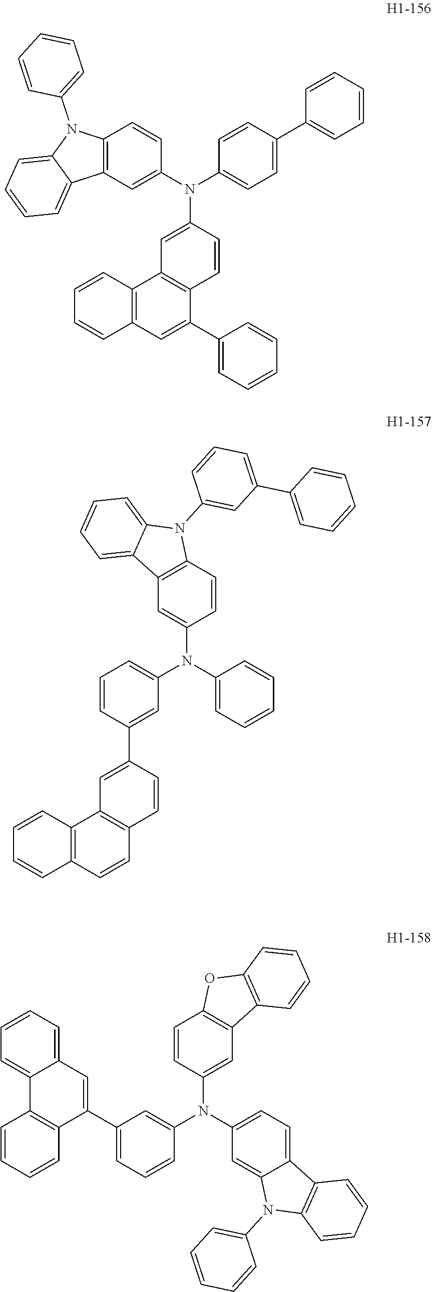 [SMILES: c1ccc(-c2ccc(N(c3ccc4c(-c5ccccc5)cc5ccccc5c4c3)c3ccc4c(c3)c3ccccc3n4-c3ccccc3)cc2)cc1 ; c1ccc(-c2cccc(-n3c4ccccc4c4cc(N(c5ccccc5)c5cccc(-c6ccc7ccc8ccccc8c7c6)c5)ccc43)c2)cc1 ; c1ccc(-n2c3ccccc3c3ccc(N(c4cccc(-c5cc6ccccc6c6ccccc56)c4)c4ccc5oc6ccccc6c5c4)cc32)cc1]

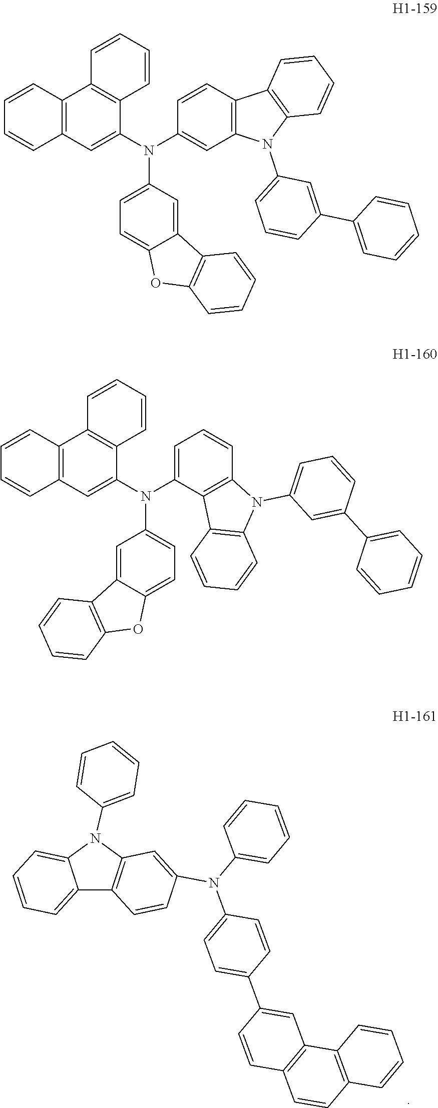 [SMILES: c1ccc(-c2cccc(-n3c4ccccc4c4ccc(N(c5ccc6oc7ccccc7c6c5)c5cc6ccccc6c6ccccc56)cc43)c2)cc1 ; c1ccc(-c2cccc(-n3c4ccccc4c4c(N(c5ccc6oc7ccccc7c6c5)c5cc6ccccc6c6ccccc56)cccc43)c2)cc1 ; c1ccc(N(c2ccc(-c3ccc4ccc5ccccc5c4c3)cc2)c2ccc3c4ccccc4n(-c4ccccc4)c3c2)cc1]

The compound represented by Formula 1 according to the present disclosure can be manufactured by referring to synthetic methods known to those skilled in the art, for example, synthetic methods disclosed in Korean Patent Application Laid-Open No. 2019-0101739, U.S. Patent Application Publication No. US 2017/0025609 A1, etc., but is not limited thereto.

According to one embodiment, the light-emitting layer comprises at least one compound represented by the following Formula 2 and at least one compound represented by the following Formula 3.

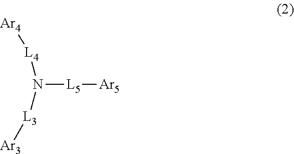

In Formula 2,
L₃ to L₅ each independently represent a single bond, a substituted or unsubstituted (C1-C30)alkylene, a substituted or unsubstituted (C6-C30)arylene, a substituted or unsubstituted (3-to 30-membered)heteroarylene, or a substituted or unsubstituted (C3-C30)cycloalkylene;
Ar₃ to Ar₅ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or —N(Ar₁₁)(Ar₁₂); and
Ar₁₁ and Ar₁₂ each independently represent a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl;
provided that, in Formula 2, the case where all of L₃ to L₅ are a single bond, and all of Ar₃ to Ar₅ are hydrogen is excluded.

In one embodiment, L₃ to L₅ each independently may be a single bond, a substituted or unsubstituted (C6-C30)arylene, or a substituted or unsubstituted (5- to 30-membered)heteroarylene, preferably a single bond, a substituted or unsubstituted (C6-C25)arylene, or a substituted or unsubstituted (5- to 25-membered)heteroarylene, more preferably a single bond, a substituted or unsubstituted (C6-C18)arylene, or a substituted or unsubstituted (5- to 18-membered)heteroarylene. For example, L₃ to L₅ each independently may be a single bond, or a phenylene unsubstituted or substituted with phenyl or pyridyl, a substituted or unsubstituted naphthylene, a substituted or unsubstituted phenanthrenylene, a substituted or unsubstituted o-biphenylene, a substituted or unsubstituted m-biphenylene, a substituted or unsubstituted p-biphenylene, a substituted or unsubstituted pyridylene, or a substituted or unsubstituted carbazolylene.

In one embodiment, Ar₃ to Ar₅ each independently may be a substituted or unsubstituted (C1-C30)alkyl, a substituted or unsubstituted (C6-C30)aryl, a substituted or unsubstituted (5-to 30-membered)heteroaryl, a substituted or unsubstituted tri(C6-C30)arylsilyl, or —N(Ar₁₁)(Ar₁₂), preferably a substituted or unsubstituted (C1-C10)alkyl, a substituted or unsubstituted (C6-C25)aryl, a substituted or unsubstituted (5- to 25-membered)heteroaryl, a substituted or unsubstituted tri(C6-C25)arylsilyl, or —N(Ar₁₁)(Ar₁₂), more preferably a substituted or unsubstituted (C1-C4)alkyl, a substituted or unsubstituted (C6-C18)aryl, a substituted or unsubstituted (5- to 18-membered)heteroaryl, a substituted or unsubstituted tri(C6-C18)arylsilyl, or —N(Ar₁₁)(Ar₁₂). Wherein, Ar₁₁ and Ar₁₂ each independently may be a substituted or unsubstituted (C6-C30)aryl or a substituted or unsubstituted (5- to 30-membered)heteroaryl, preferably a substituted or unsubstituted (C6-C25)aryl or a substituted or unsubstituted (5-to 25-membered)heteroaryl, more preferably a substituted or unsubstituted (C6-C18)aryl or a substituted or unsubstituted (5- to 18-membered)heteroaryl. For example, Ar₃ to Ar₅ each independently may be a phenyl unsubstituted or substituted with deuterium or cyano, an isopropyl unsubstituted or substituted with phenyl, a substituted or unsubstituted naphthyl, a substituted or unsubstituted m-biphenyl, a substituted or unsubstituted p-biphenyl, a substituted or unsubstituted o-terphenyl, a substituted or unsubstituted m-terphenyl, a substituted or unsubstituted p-terphenyl, a substituted or unsubstituted o-quarterphenyl, a substituted or unsubstituted phenanthrenyl, a substituted or unsubstituted chrysenyl, fluorenyl unsubstituted or substituted with at least one methyl, fluorenyl unsubstituted or substituted with at least one phenyl, a substituted or unsubstituted 9,9,10,10-tetramethylphenanthrenyl, a substituted or unsubstituted triphenylsilyl, a substituted or unsubstituted pyridyl, carbazolyl, dibenzofuranyl unsubstituted or substituted with deuterium, a substituted or unsubstituted dibenzothiophenyl, a substituted or unsubstituted phenanthrolinyl, a substituted or unsubstituted dibenzoselenophenyl, or a substituted or unsubstituted naphthobenzoselenophenyl. Wherein, the substituents, for example, may be further substituted with at least one of deuterium, methyl, and phenyl.

For example, Ar₁₁ and Ar₁₂ each independently may be a phenyl unsubstituted or substituted with diphenylamino, a substituted or unsubstituted naphthyl, a substituted or unsubstituted phenanthrenyl, a substituted or unsubstituted o-biphenyl, a substituted or unsubstituted m-biphenyl, a substituted or unsubstituted p-biphenyl, a substituted or unsubstituted o-terphenyl, a substituted or unsubstituted m-terphenyl, a substituted or unsubstituted p-terphenyl, a substituted or unsubstituted pyridyl, a substituted or unsubstituted dibenzothiophenyl, or a substituted or unsubstituted dibenzofuranyl.

According to one embodiment, at least one of Ar₃ to Ar₅ may be represented by any one of the following Formulas 2-1 to 2-3.

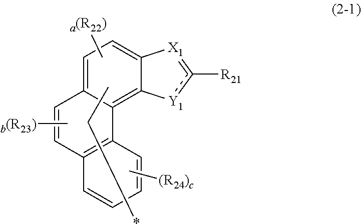

In Formula 2-1,

X₁ and Y₁ each independently represent —N═, —NR₂₅—, —O—, or —S—; provided that any one of X₁ and Y₁ is —N═, and the other of X₁ and Y₁ is —NR₂₅—, —O—, or —S—;

R₂₁ represents a substituted or unsubstituted (C6-C30)aryl or a substituted or unsubstituted (3- to 30-membered)heteroaryl;

R₂₂ to R₂₅ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to the adjacent substituents to form a ring(s);
a and b each independently represent an integer of 1 or 2, and c represents an integer of 1 to 3;
and when a to c are an integer of 2 or more, each of R₂₂ to each of R₂₄ may be the same as or different from each other; and
* indicates a connection position connected to L₃ to L₅ in Formula 2.

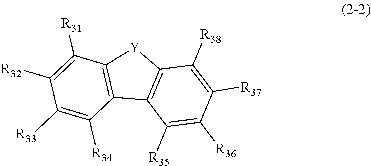

In Formula 2-2,
Y represents —O—, —S—, or —NR₃₉;
R₃₉ represents a substituted or unsubstituted (C6-C30)aryl or a substituted or unsubstituted (3- to 30-membered)heteroaryl; and
R₃₁ to R₃₈ each independently represent a connection position connected to L₃ to L₅ in Formula 2; or hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to the adjacent substituents to form a ring(s).

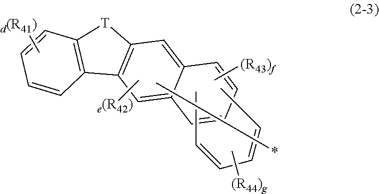

In Formula 2-3,
T represents —O—, —S—, —CR₄₅R₄₆, —NR₄₇, or —Se—;
R₄₅ to R₄₇ each independently represent hydrogen, deuterium, a halogen, a substituted or unsubstituted (C1-C30)alkyl, or a substituted or unsubstituted (C6-C30)aryl;
R₄₁ to R₄₄ each independently represent hydrogen, deuterium, a halogen, a substituted or unsubstituted (C1-C30)alkyl, a substituted or unsubstituted (C6-C30)aryl, a substituted or unsubstituted (3- to 30-membered)heteroaryl, or —N(Ar₂₁)(Ar₂₂); or may be linked to the adjacent substituents to form a ring(s);
Ar₂₁ and Ar₂₂ each independently represent a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl;
d and g each independently represent an integer of 1 to 4, and e and f each independently represent an integer of 1 or 2;
when d to g are an integer of 2 or more, each of R₄₁ to each of R₄₄ may be the same as or different from each other; and
* indicates a connection position connected to L₃ to L₅ in Formula 2.

According to one embodiment, the compound represented by Formula 2-3 may be represented by the following Formula 2-3-1 or 2-3-2.

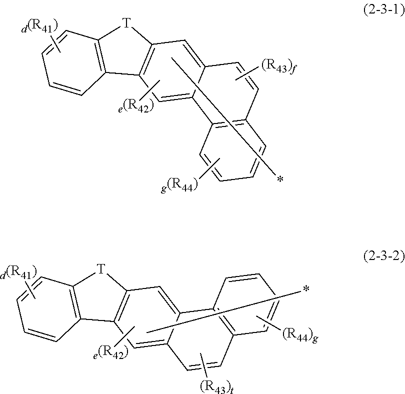

In Formulas 2-3-1 and 2-3-2,
T, R₄₁ to R₄₄, and d to g are as defined in Formula 2-3.
In Formula 2-1, R₂₁ may be a substituted or unsubstituted (C6-C30)aryl or a substituted or unsubstituted (5- to 30-membered)heteroaryl, for example, a phenyl unsubstituted or substituted with deuterium, a substituted or unsubstituted naphthyl, a substituted or unsubstituted o-biphenyl, a substituted or unsubstituted m-biphenyl, a substituted or unsubstituted p-biphenyl, or a substituted or unsubstituted pyridyl.

In Formula 2-1, R₂₂ to R₂₅ each independently may be hydrogen, deuterium, halogen, cyano, a substituted or unsubstituted (C6-C30)aryl, or a substituted or unsubstituted (5- to 30-membered)heteroaryl, preferably hydrogen, deuterium, a substituted or unsubstituted (C6-C25)aryl, or a substituted or unsubstituted (5- to 25-membered)heteroaryl, more preferably hydrogen, deuterium, a substituted or unsubstituted (C6-C18)aryl, or a substituted or unsubstituted (5- to 18-membered)heteroaryl. For example, R₂₂ to R₂₅ each independently may be hydrogen, deuterium, a phenyl unsubstituted or substituted with naphthyl or triphenylsilyl, a substituted or unsubstituted naphthyl, a substituted or unsubstituted o-biphenyl, a substituted or unsubstituted m-biphenyl, a substituted or unsubstituted p-biphenyl, a substituted or unsubstituted p-terphenyl, fluorenyl unsubstituted or substituted with at least one methyl, a substituted or unsubstituted dibenzofuranyl, a substituted or unsubstituted dibenzothiophenyl, a substituted or unsubstituted benzonaphthofuranyl, or a substituted or unsubstituted benzonaphthothiophenyl.

In Formula 2-2, one of R₃₁ to R₃₈ may be a connection position connected to L₃ to L₅ in Formula 2.

In Formula 2-3, T may be —O— or —S—.

In Formula 2-3, R₄₁ to R₄₄ each independently may be hydrogen or deuterium, a substituted or unsubstituted (C6-C30)aryl, or —N(Ar₂₁)(Ar₂₂), for example, hydrogen, deuterium, a substituted or unsubstituted phenyl, a substituted or unsubstituted p-biphenyl, a substituted or unsubstituted m-biphenyl, a substituted or unsubstituted naphthyl, or a substituted or unsubstituted diphenylamine. For example, Ar₂₁ and Ar₂₂ each independently may be a substituted or unsubstituted phenyl.

According to one embodiment, the compound represented by Formula 2 may be more specifically illustrated by the following compounds, but is not limited thereto:

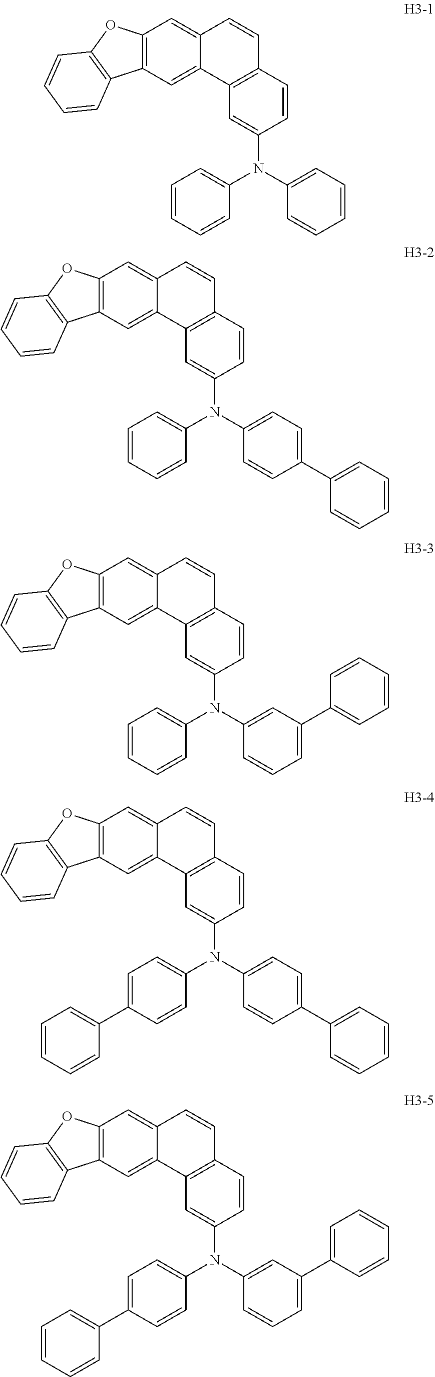

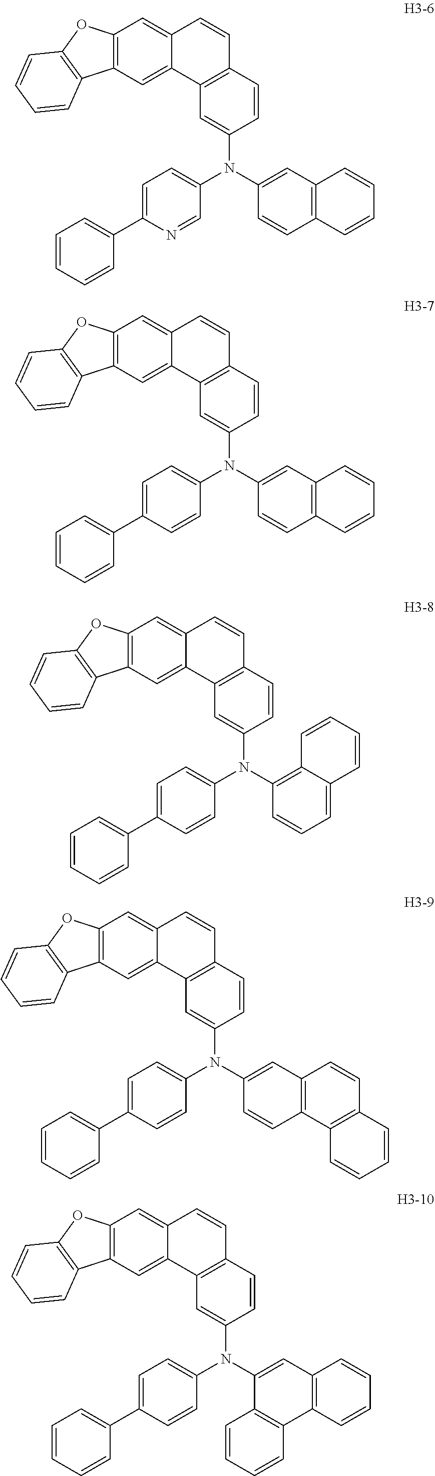

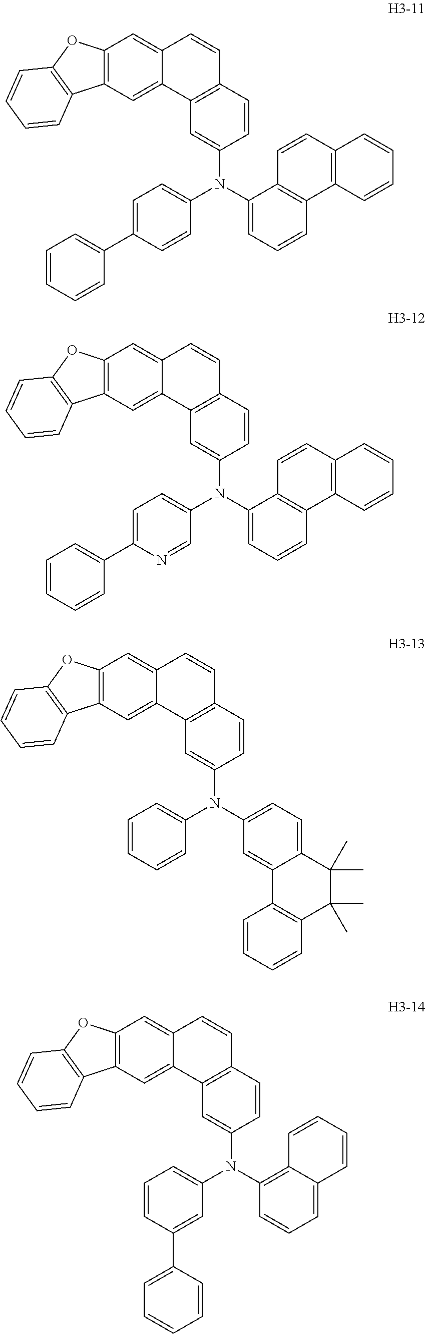 [SMILES: c1ccc(-c2ccc(N(c3ccc4ccc5cc6oc7ccccc7c6cc5c4c3)c3cccc4c3ccc3ccccc34)cc2)cc1 ; c1ccc(-c2ccc(N(c3ccc4ccc5cc6oc7ccccc7c6cc5c4c3)c3cccc4c3ccc3ccccc34)cn2)cc1 ; CC1(C)c2ccccc2-c2cc(N(c3ccccc3)c3ccc4ccc5cc6oc7ccccc7c6cc5c4c3)ccc2C1(C)C ; c1ccc(-c2cccc(N(c3ccc4ccc5cc6oc7ccccc7c6cc5c4c3)c3cccc4ccccc34)c2)cc1]

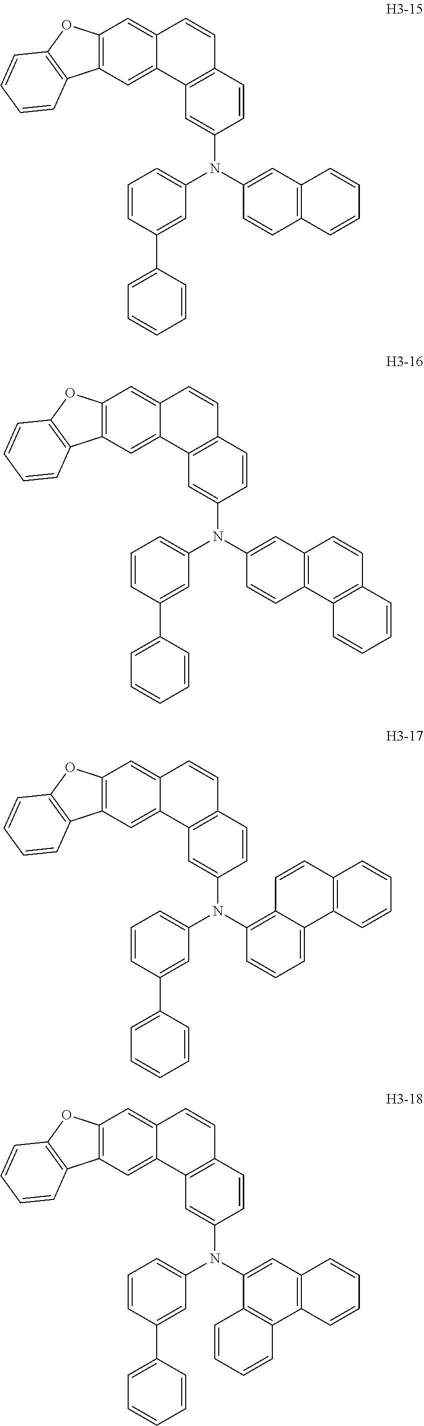

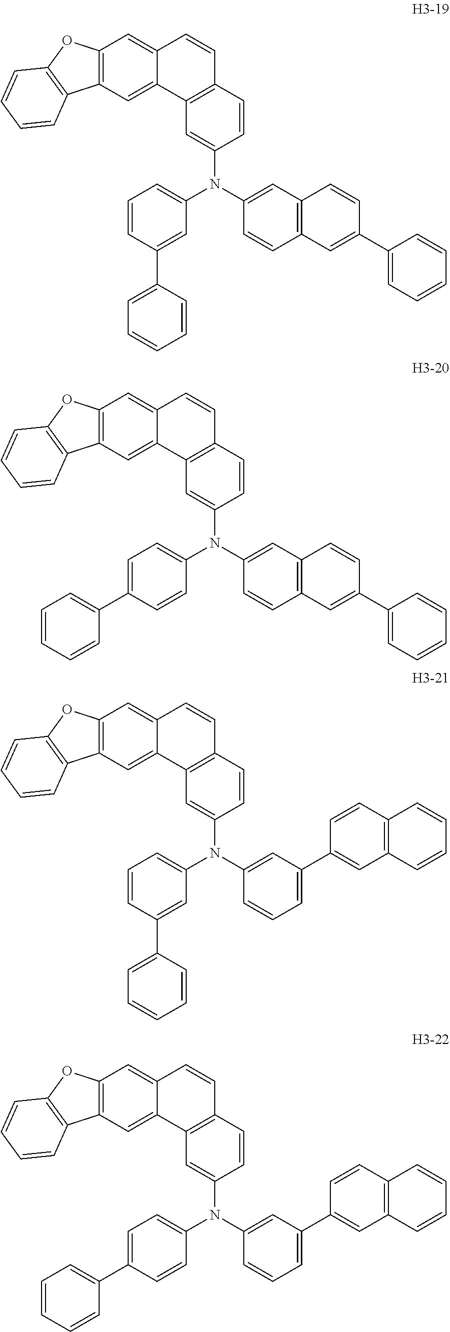 [SMILES: c1ccc(-c2cccc(N(c3ccc4cc(-c5ccccc5)ccc4c3)c3ccc4ccc5cc6oc7ccccc7c6cc5c4c3)c2)cc1 ; c1ccc(-c2ccc(N(c3ccc4cc(-c5ccccc5)ccc4c3)c3ccc4ccc5cc6oc7ccccc7c6cc5c4c3)cc2)cc1 ; c1ccc(-c2cccc(N(c3cccc(-c4ccc5ccccc5c4)c3)c3ccc4ccc5cc6oc7ccccc7c6cc5c4c3)c2)cc1 ; c1ccc(-c2ccc(N(c3cccc(-c4ccc5ccccc5c4)c3)c3ccc4ccc5cc6oc7ccccc7c6cc5c4c3)cc2)cc1]

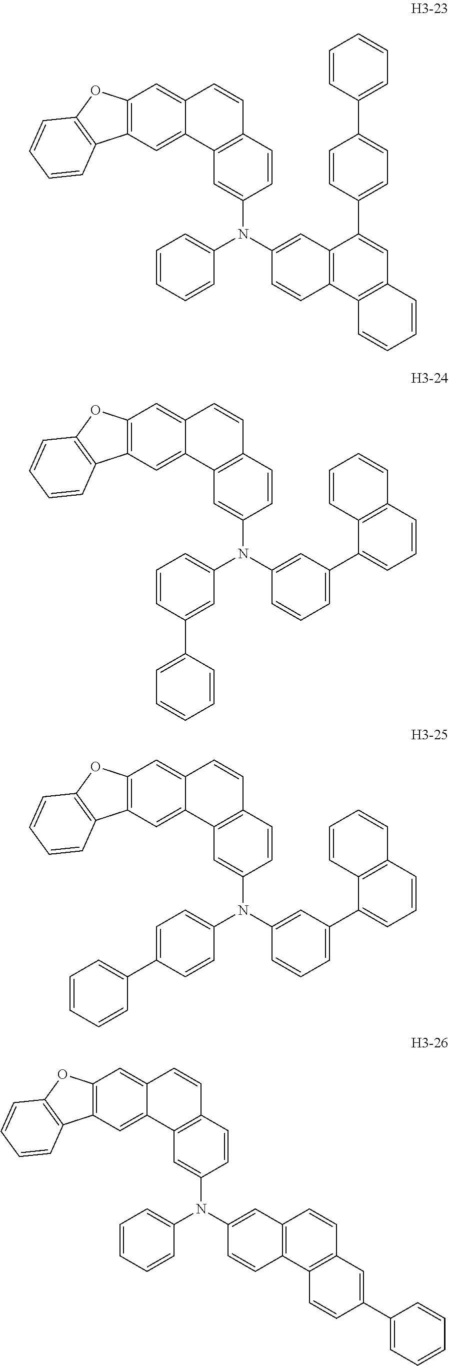

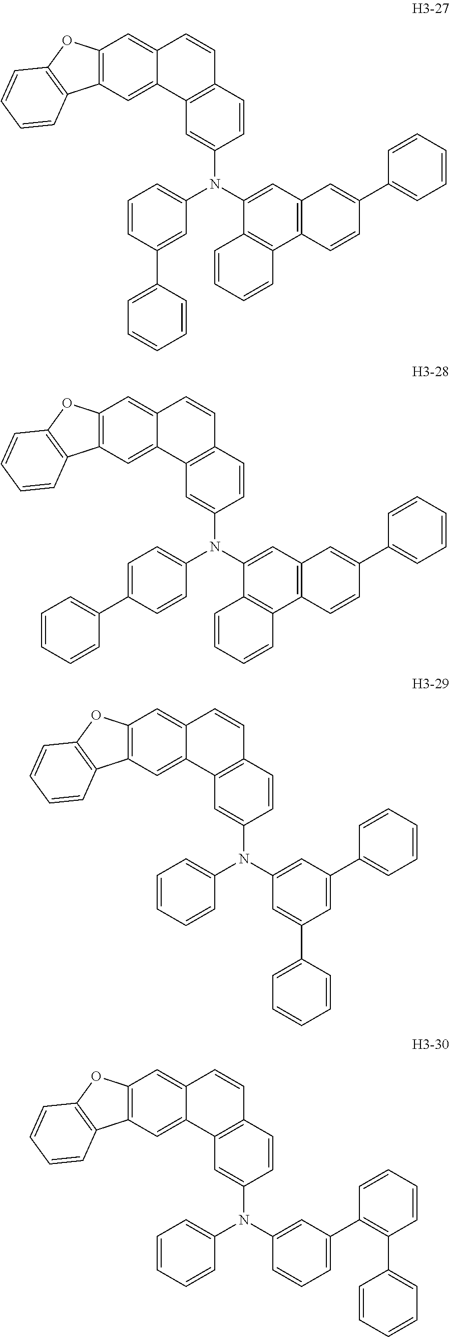 [SMILES: c1ccc(-c2cccc(N(c3ccc4ccc5cc6oc7ccccc7c6cc5c4c3)c3cc4cc(-c5ccccc5)ccc4c4ccccc34)c2)cc1 ; c1ccc(-c2ccc(N(c3ccc4ccc5cc6oc7ccccc7c6cc5c4c3)c3cc4cc(-c5ccccc5)ccc4c4ccccc34)cc2)cc1 ; c1ccc(-c2cc(-c3ccccc3)cc(N(c3ccccc3)c3ccc4ccc5cc6oc7ccccc7c6cc5c4c3)c2)cc1 ; c1ccc(-c2ccccc2-c2cccc(N(c3ccccc3)c3ccc4ccc5cc6oc7ccccc7c6cc5c4c3)c2)cc1]

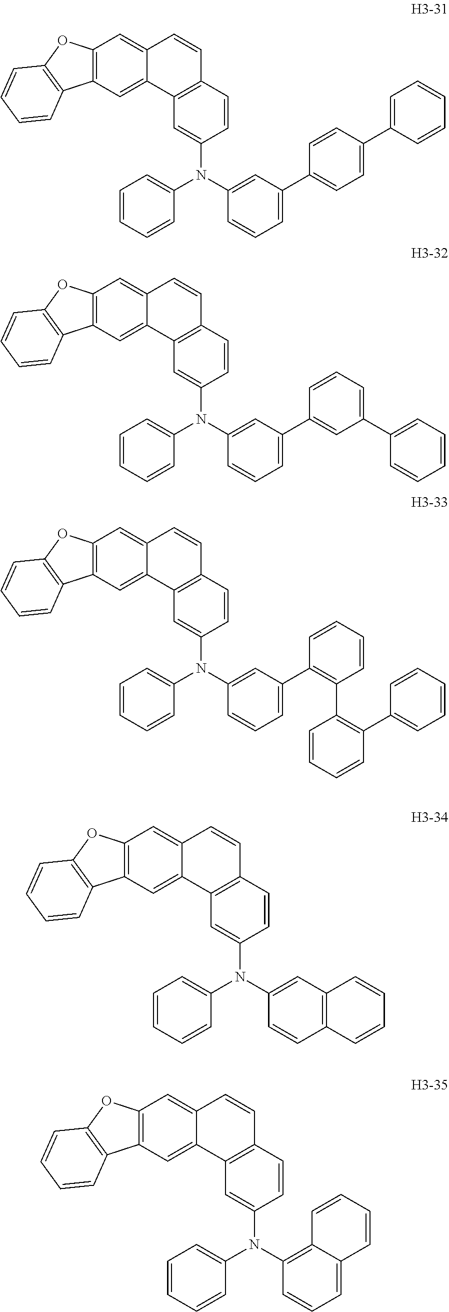 [SMILES: c1ccc(-c2ccc(-c3cccc(N(c4ccccc4)c4ccc5ccc6cc7oc8ccccc8c7cc6c5c4)c3)cc2)cc1 ; c1ccc(-c2cccc(-c3cccc(N(c4ccccc4)c4ccc5ccc6cc7oc8ccccc8c7cc6c5c4)c3)c2)cc1 ; c1ccc(-c2ccccc2-c2ccccc2-c2cccc(N(c3ccccc3)c3ccc4ccc5cc6oc7ccccc7c6cc5c4c3)c2)cc1 ; c1ccc(N(c2ccc3ccccc3c2)c2ccc3ccc4cc5oc6ccccc6c5cc4c3c2)cc1 ; c1ccc(N(c2ccc3ccc4cc5oc6ccccc6c5cc4c3c2)c2cccc3ccccc23)cc1]

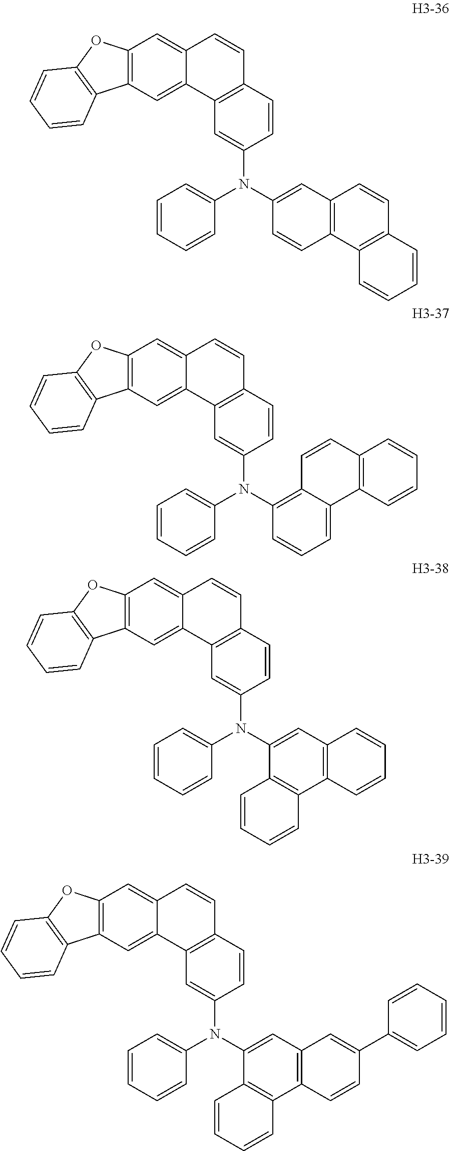

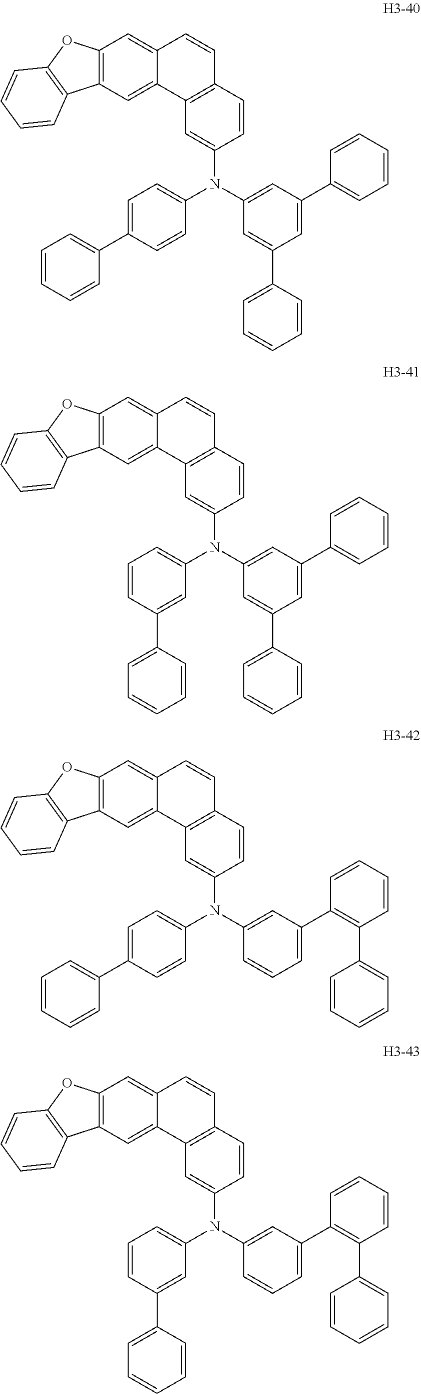 [SMILES: c1ccc(-c2ccc(N(c3cc(-c4ccccc4)cc(-c4ccccc4)c3)c3ccc4ccc5cc6oc7ccccc7c6cc5c4c3)cc2)cc1 ; c1ccc(-c2cccc(N(c3cc(-c4ccccc4)cc(-c4ccccc4)c3)c3ccc4ccc5cc6oc7ccccc7c6cc5c4c3)c2)cc1 ; c1ccc(-c2ccc(N(c3cccc(-c4ccccc4-c4ccccc4)c3)c3ccc4ccc5cc6oc7ccccc7c6cc5c4c3)cc2)cc1 ; c1ccc(-c2cccc(N(c3cccc(-c4ccccc4-c4ccccc4)c3)c3ccc4ccc5cc6oc7ccccc7c6cc5c4c3)c2)cc1]

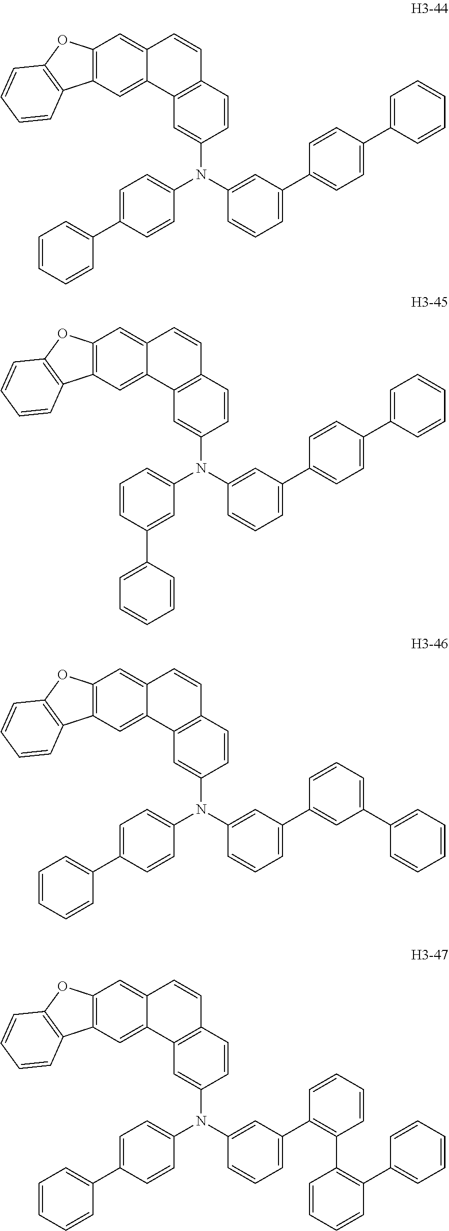 [SMILES: c1ccc(-c2ccc(-c3cccc(N(c4ccc(-c5ccccc5)cc4)c4ccc5ccc6cc7oc8ccccc8c7cc6c5c4)c3)cc2)cc1 ; c1ccc(-c2ccc(-c3cccc(N(c4cccc(-c5ccccc5)c4)c4ccc5ccc6cc7oc8ccccc8c7cc6c5c4)c3)cc2)cc1 ; c1ccc(-c2ccc(N(c3cccc(-c4cccc(-c5ccccc5)c4)c3)c3ccc4ccc5cc6oc7ccccc7c6cc5c4c3)cc2)cc1 ; c1ccc(-c2ccc(N(c3cccc(-c4ccccc4-c4ccccc4-c4ccccc4)c3)c3ccc4ccc5cc6oc7ccccc7c6cc5c4c3)cc2)cc1]

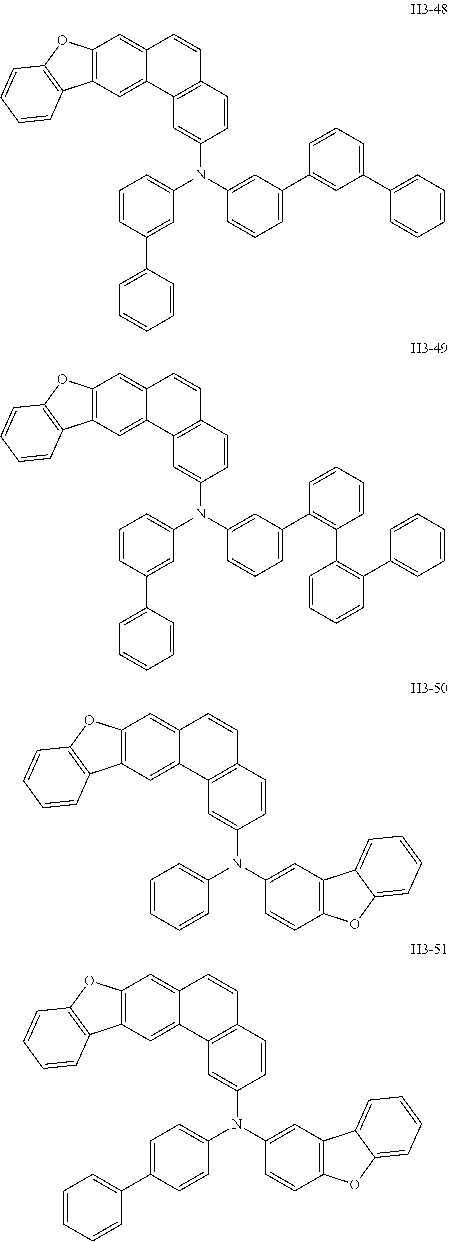

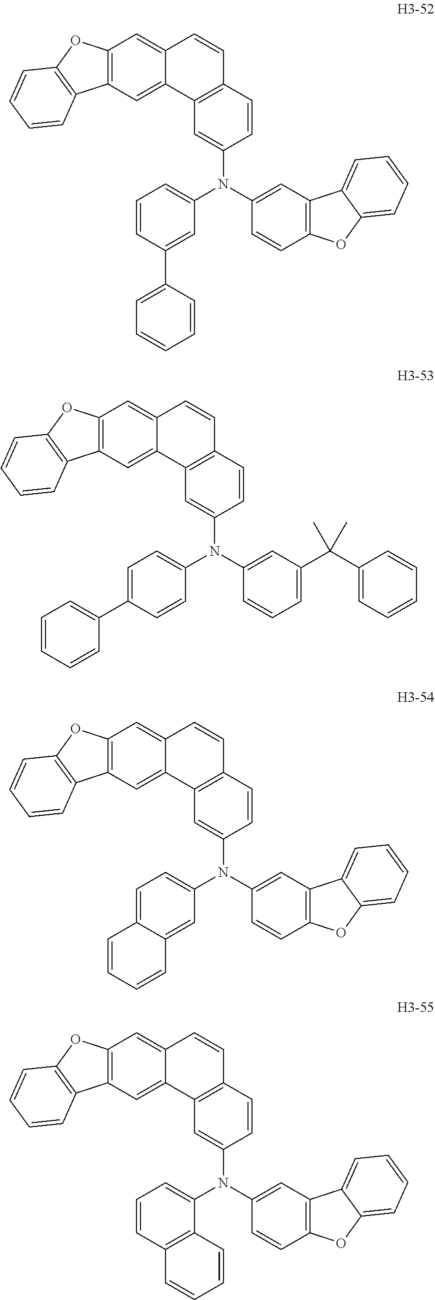

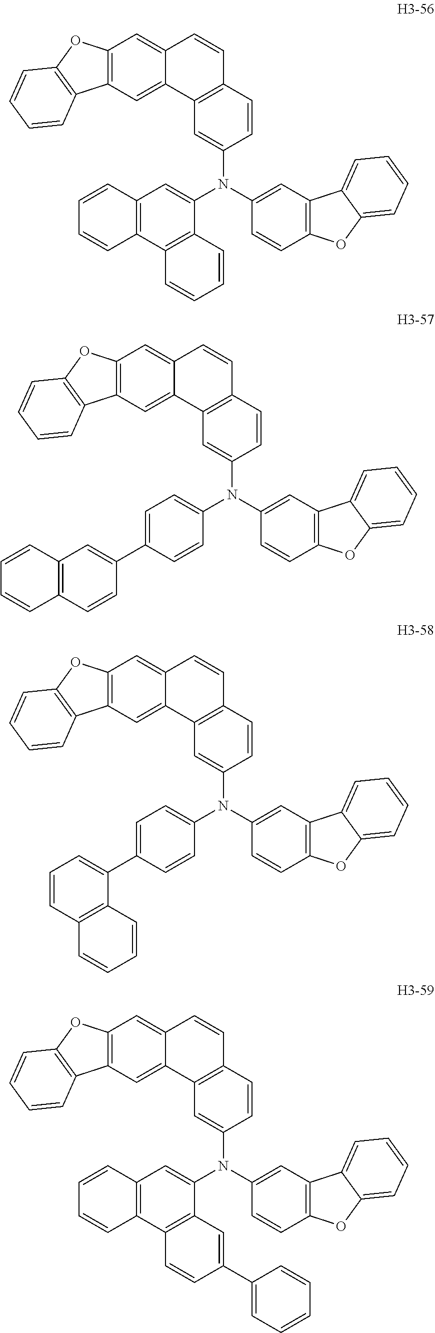

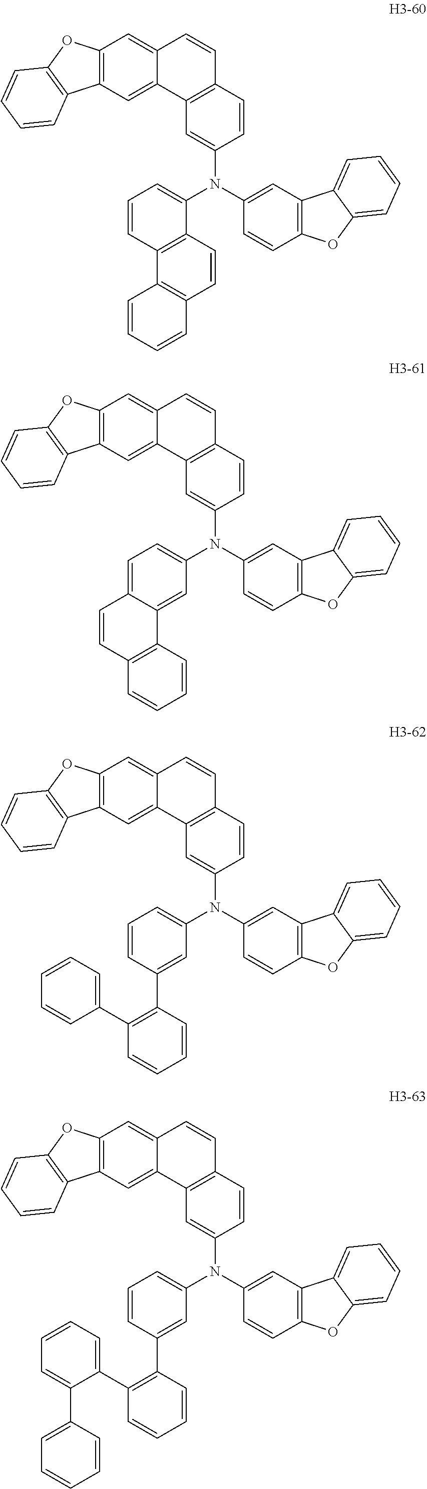 [SMILES: c1ccc2c(c1)ccc1c(N(c3ccc4ccc5cc6oc7ccccc7c6cc5c4c3)c3ccc4oc5ccccc5c4c3)cccc12 ; c1ccc2c(c1)ccc1ccc(N(c3ccc4ccc5cc6oc7ccccc7c6cc5c4c3)c3ccc4oc5ccccc5c4c3)cc12 ; c1ccc(-c2ccccc2-c2cccc(N(c3ccc4ccc5cc6oc7ccccc7c6cc5c4c3)c3ccc4oc5ccccc5c4c3)c2)cc1 ; c1ccc(-c2ccccc2-c2ccccc2-c2cccc(N(c3ccc4ccc5cc6oc7ccccc7c6cc5c4c3)c3ccc4oc5ccccc5c4c3)c2)cc1]

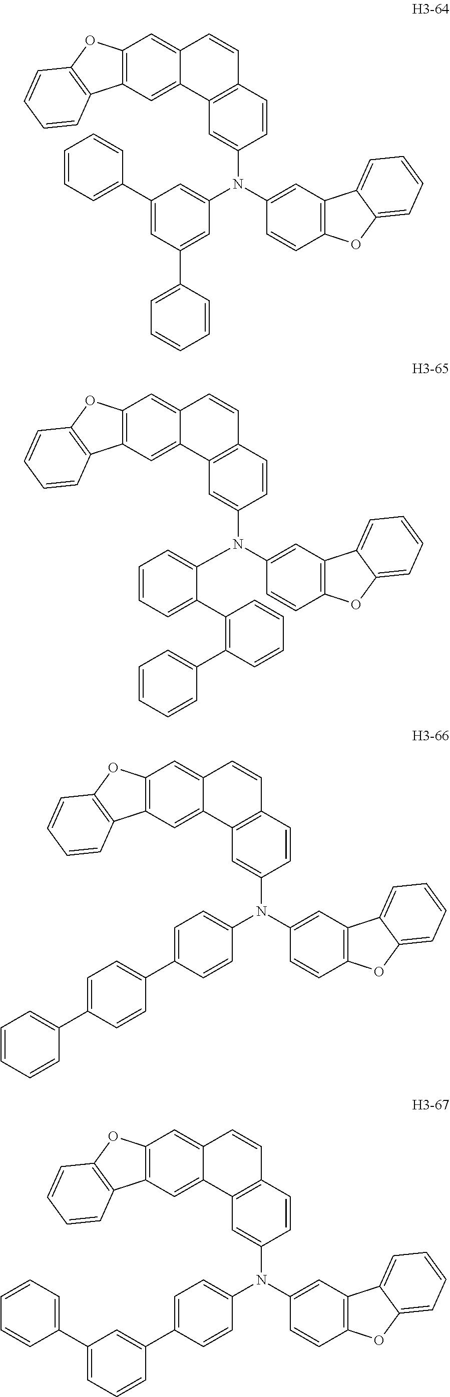

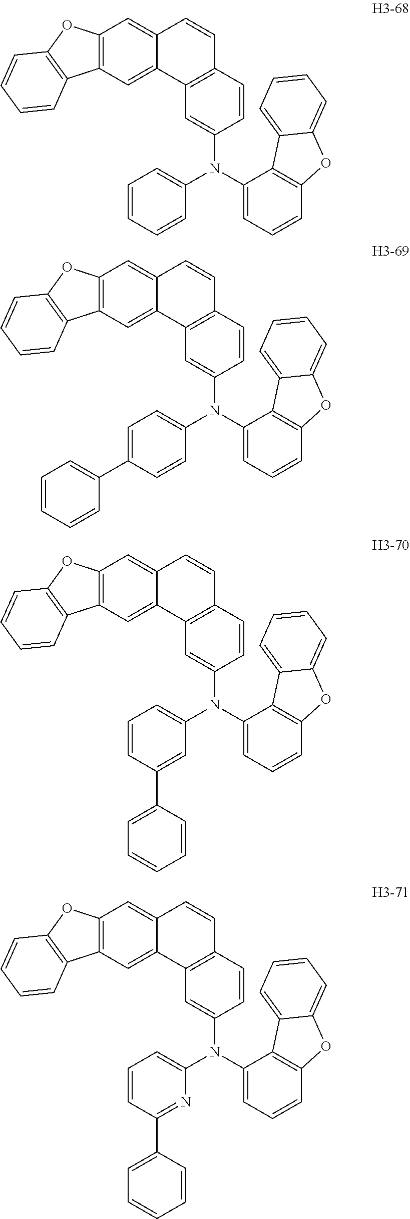

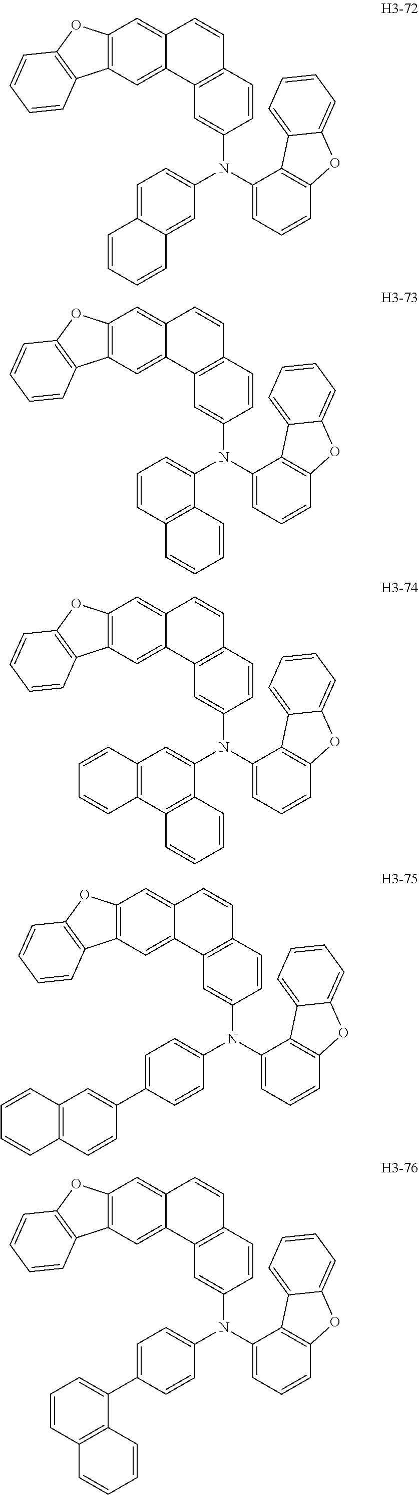

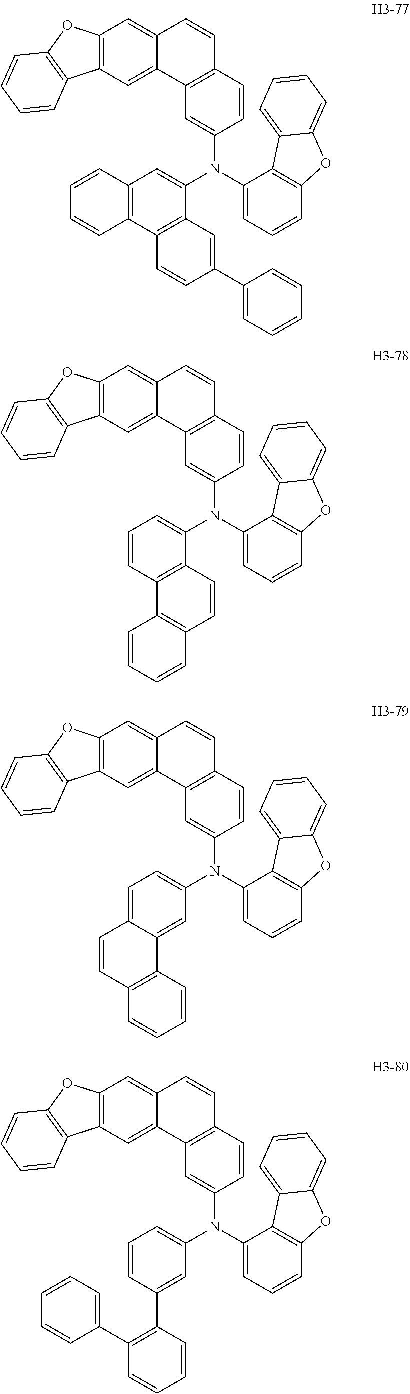 [SMILES: c1ccc(-c2ccc3c(c2)c(N(c2ccc4ccc5cc6oc7ccccc7c6cc5c4c2)c2cccc4oc5ccccc5c24)cc2ccccc23)cc1 ; c1ccc2c(c1)ccc1c(N(c3ccc4ccc5cc6oc7ccccc7c6cc5c4c3)c3cccc4oc5ccccc5c34)cccc12 ; c1ccc2c(c1)ccc1ccc(N(c3ccc4ccc5cc6oc7ccccc7c6cc5c4c3)c3cccc4oc5ccccc5c34)cc12 ; c1ccc(-c2ccccc2-c2cccc(N(c3ccc4ccc5cc6oc7ccccc7c6cc5c4c3)c3cccc4oc5ccccc5c34)c2)cc1]

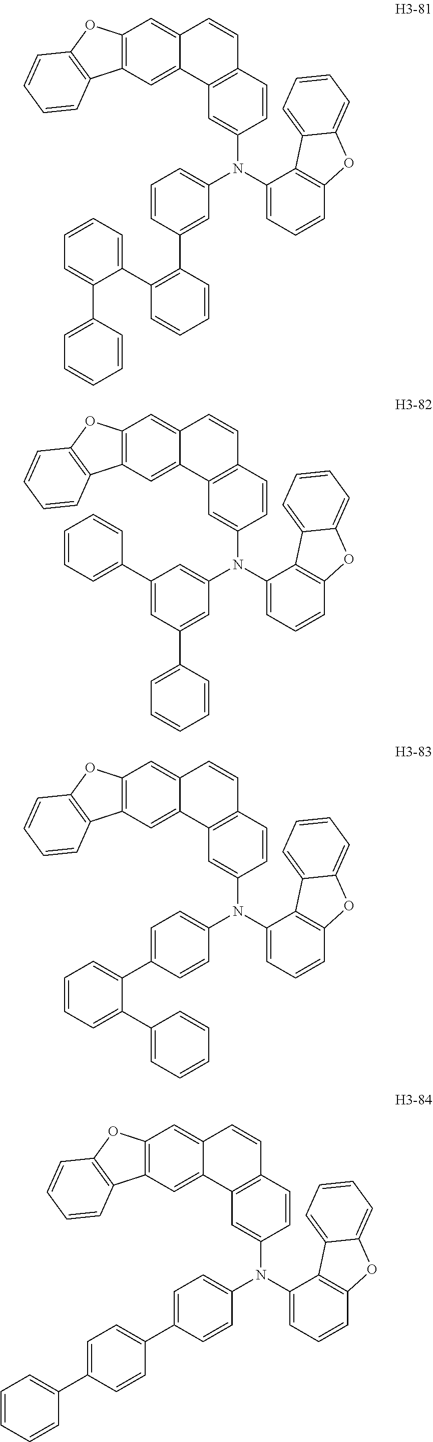

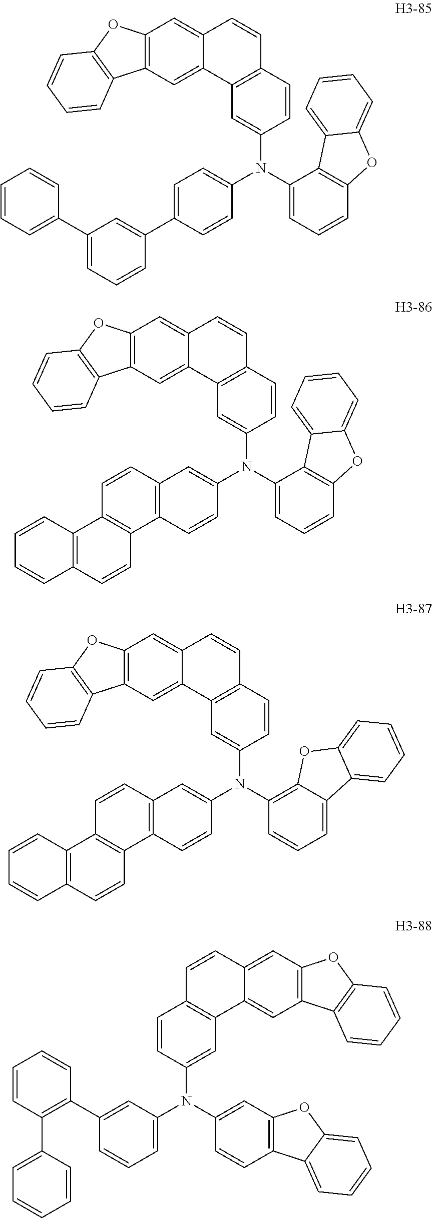

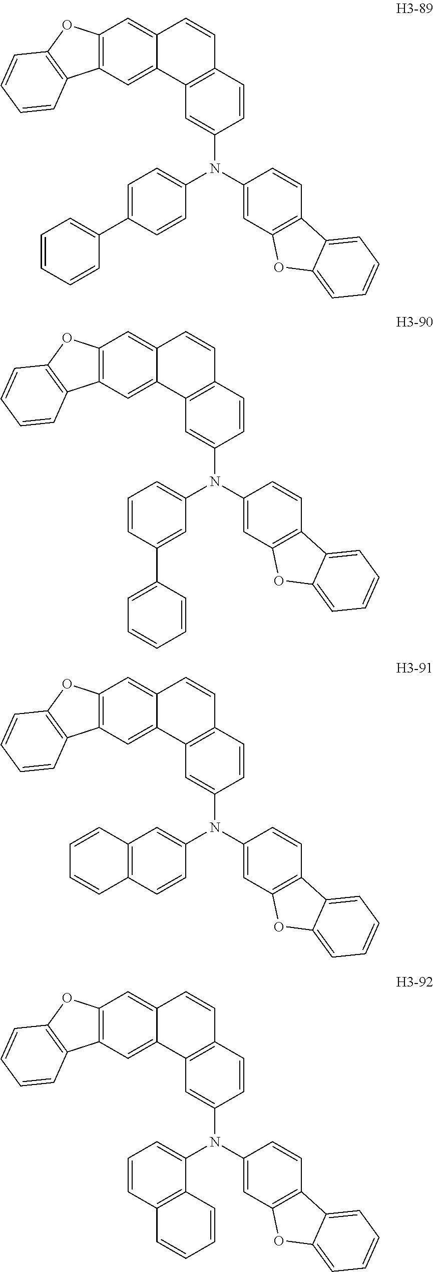

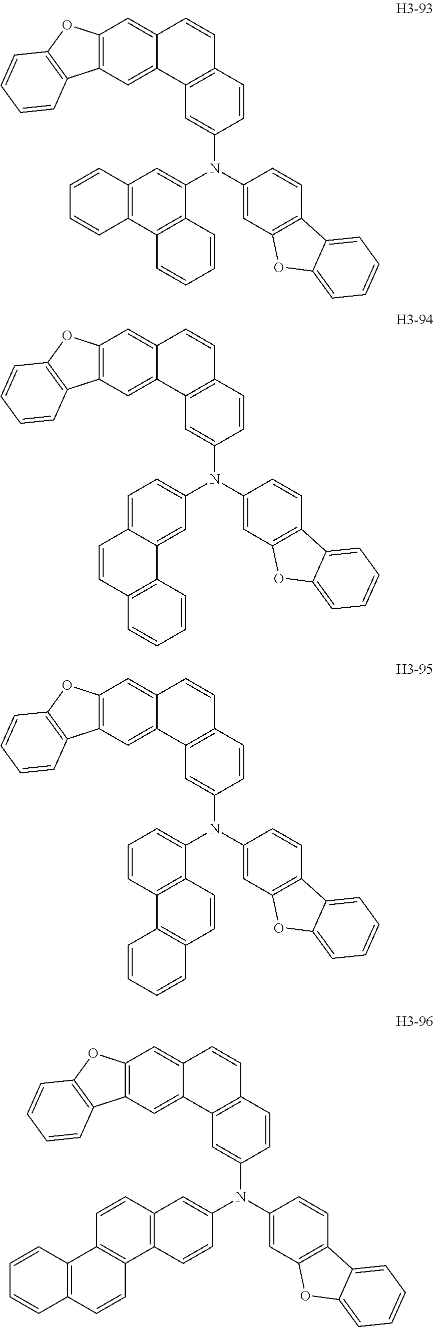

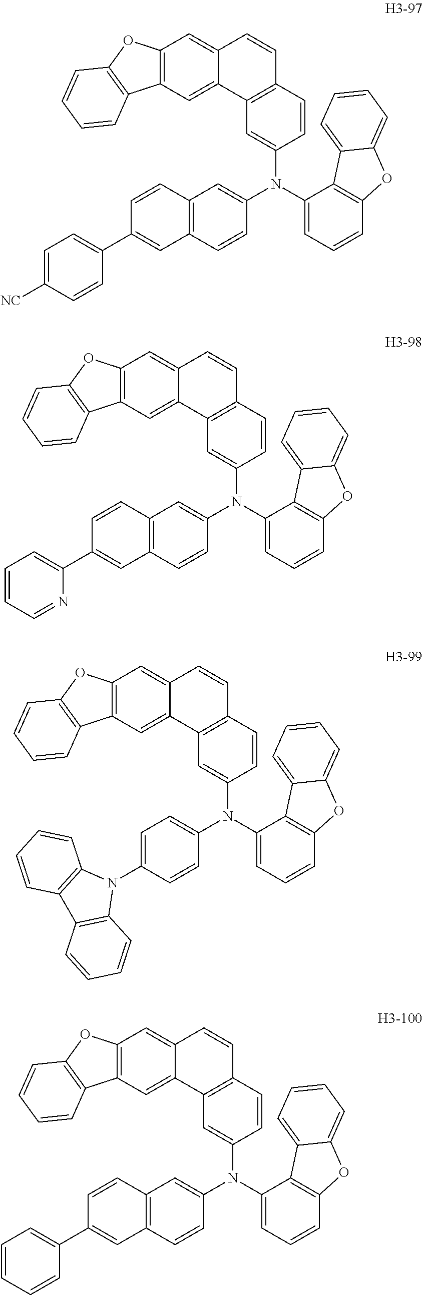 [SMILES: N#Cc1ccc(-c2ccc3cc(N(c4ccc5ccc6cc7oc8ccccc8c7cc6c5c4)c4cccc5oc6ccccc6c45)ccc3c2)cc1 ; c1ccc(-c2ccc3cc(N(c4ccc5ccc6cc7oc8ccccc8c7cc6c5c4)c4cccc5oc6ccccc6c45)ccc3c2)nc1 ; c1ccc2c(c1)oc1cc3ccc4ccc(N(c5ccc(-n6c7ccccc7c7ccccc76)cc5)c5cccc6oc7ccccc7c56)cc4c3cc12 ; c1ccc(-c2ccc3cc(N(c4ccc5ccc6cc7oc8ccccc8c7cc6c5c4)c4cccc5oc6ccccc6c45)ccc3c2)cc1]

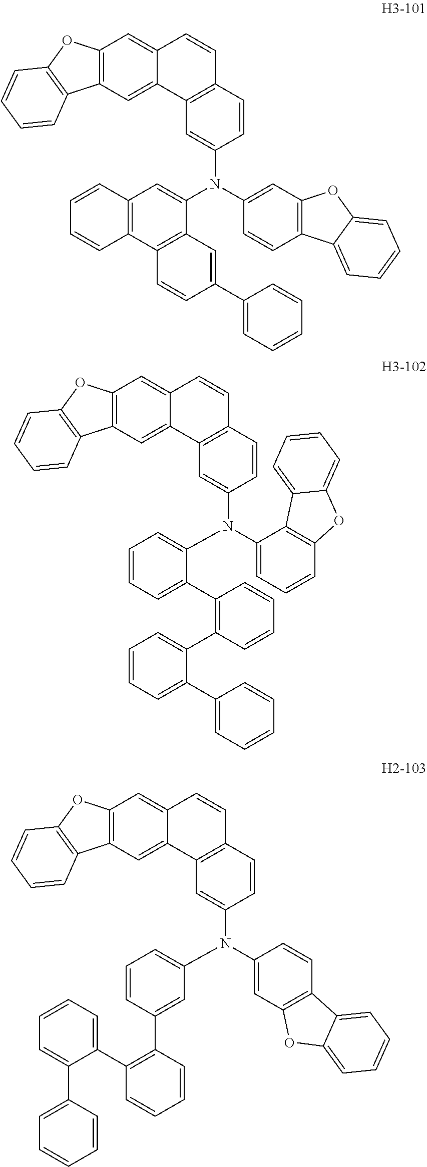 [SMILES: c1ccc(-c2ccc3c(c2)c(N(c2ccc4c(c2)oc2ccccc24)c2ccc4ccc5cc6oc7ccccc7c6cc5c4c2)cc2ccccc23)cc1 ; c1ccc(-c2ccccc2-c2ccccc2-c2ccccc2N(c2ccc3ccc4cc5oc6ccccc6c5cc4c3c2)c2cccc3oc4ccccc4c23)cc1 ; c1ccc(-c2ccccc2-c2ccccc2-c2cccc(N(c3ccc4c(c3)oc3ccccc34)c3ccc4ccc5cc6oc7ccccc7c6cc5c4c3)c2)cc1]

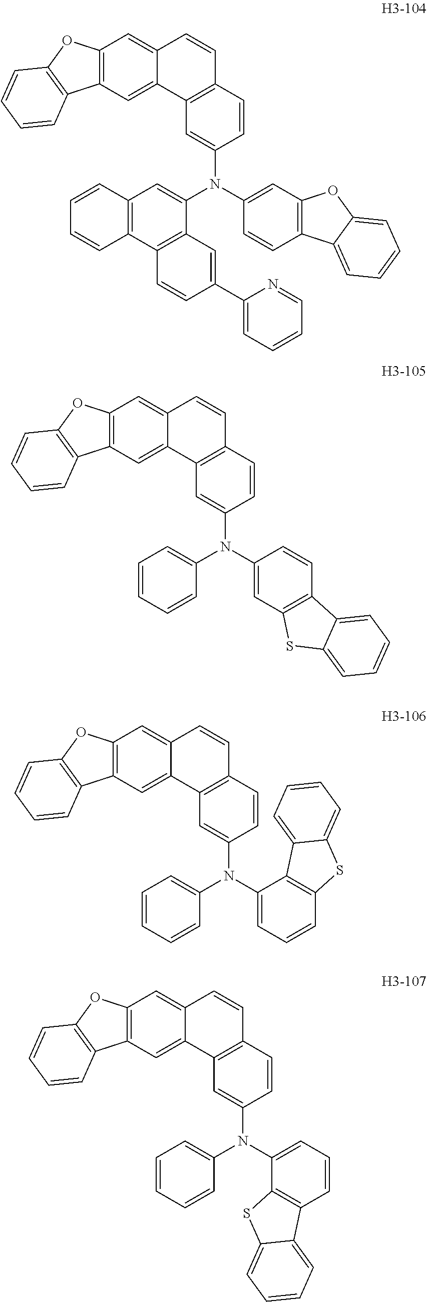 [SMILES: c1ccc(-c2ccc3c(c2)c(N(c2ccc4c(c2)oc2ccccc24)c2ccc4ccc5cc6oc7ccccc7c6cc5c4c2)cc2ccccc23)nc1 ; c1ccc(N(c2ccc3c(c2)sc2ccccc23)c2ccc3ccc4cc5oc6ccccc6c5cc4c3c2)cc1 ; c1ccc(N(c2ccc3ccc4cc5oc6ccccc6c5cc4c3c2)c2cccc3sc4ccccc4c23)cc1 ; c1ccc(N(c2ccc3ccc4cc5oc6ccccc6c5cc4c3c2)c2cccc3c2sc2ccccc23)cc1]

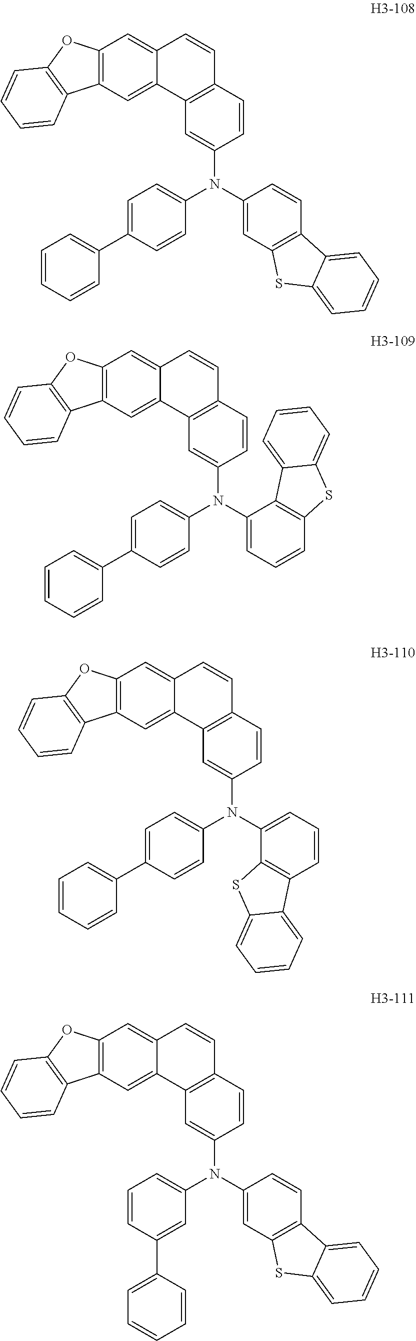 [SMILES: c1ccc(-c2ccc(N(c3ccc4c(c3)sc3ccccc34)c3ccc4ccc5cc6oc7ccccc7c6cc5c4c3)cc2)cc1 ; c1ccc(-c2ccc(N(c3ccc4ccc5cc6oc7ccccc7c6cc5c4c3)c3cccc4sc5ccccc5c34)cc2)cc1 ; c1ccc(-c2ccc(N(c3ccc4ccc5cc6oc7ccccc7c6cc5c4c3)c3cccc4c3sc3ccccc34)cc2)cc1 ; c1ccc(-c2cccc(N(c3ccc4c(c3)sc3ccccc34)c3ccc4ccc5cc6oc7ccccc7c6cc5c4c3)c2)cc1]

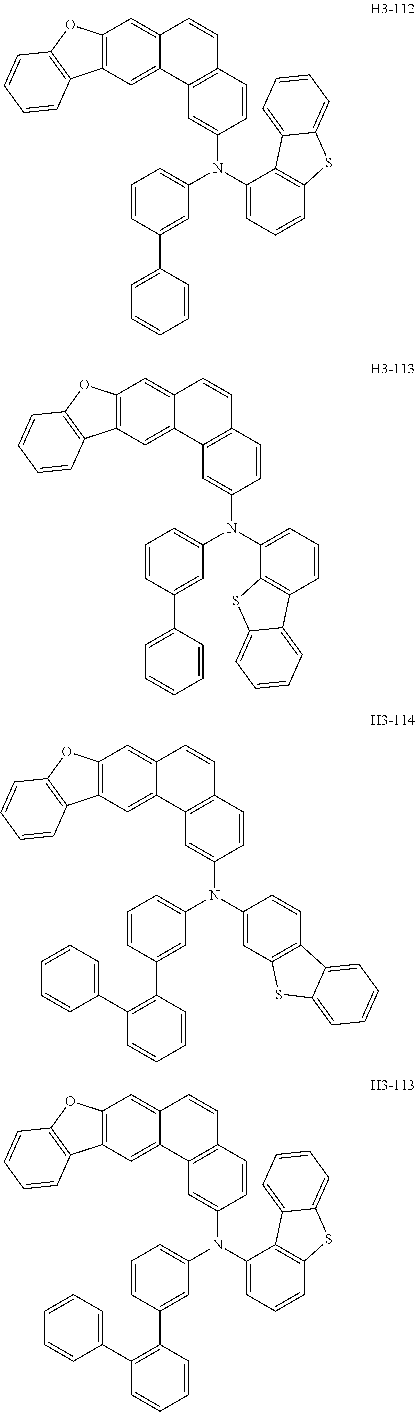

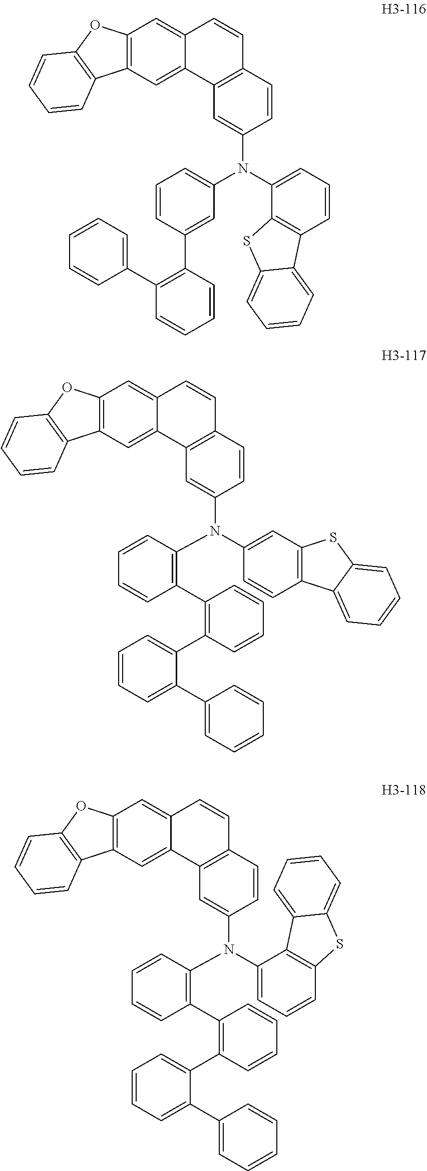

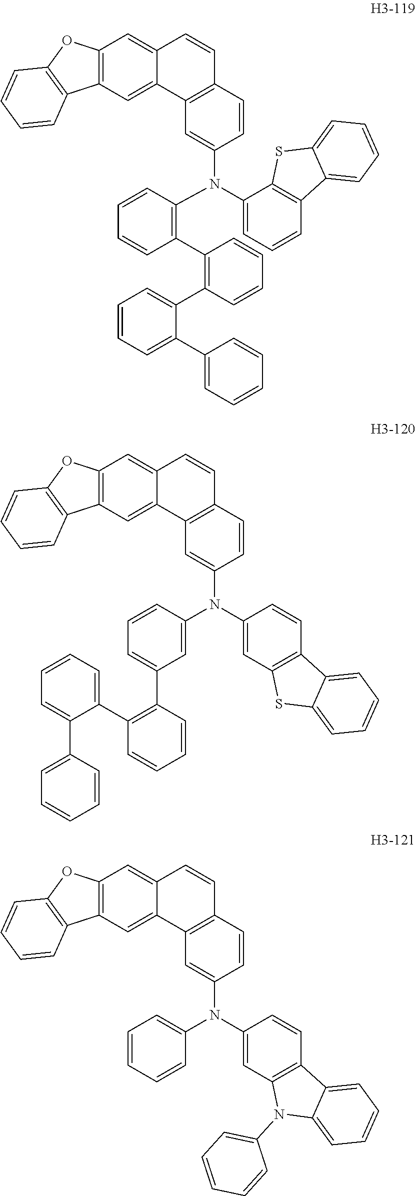 [SMILES: c1ccc(-c2ccccc2-c2ccccc2-c2ccccc2N(c2ccc3ccc4cc5oc6ccccc6c5cc4c3c2)c2cccc3c2sc2ccccc23)cc1 ; c1ccc(-c2ccccc2-c2ccccc2-c2cccc(N(c3ccc4c(c3)sc3ccccc34)c3ccc4ccc5cc6oc7ccccc7c6cc5c4c3)c2)cc1 ; c1ccc(N(c2ccc3ccc4cc5oc6ccccc6c5cc4c3c2)c2ccc3c4ccccc4n(-c4ccccc4)c3c2)cc1]

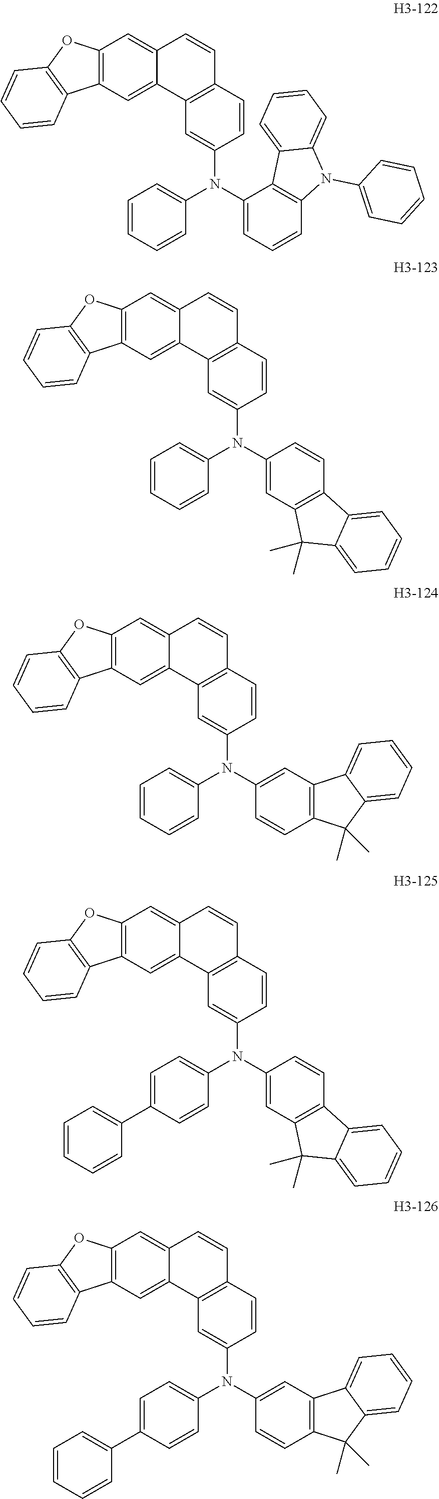

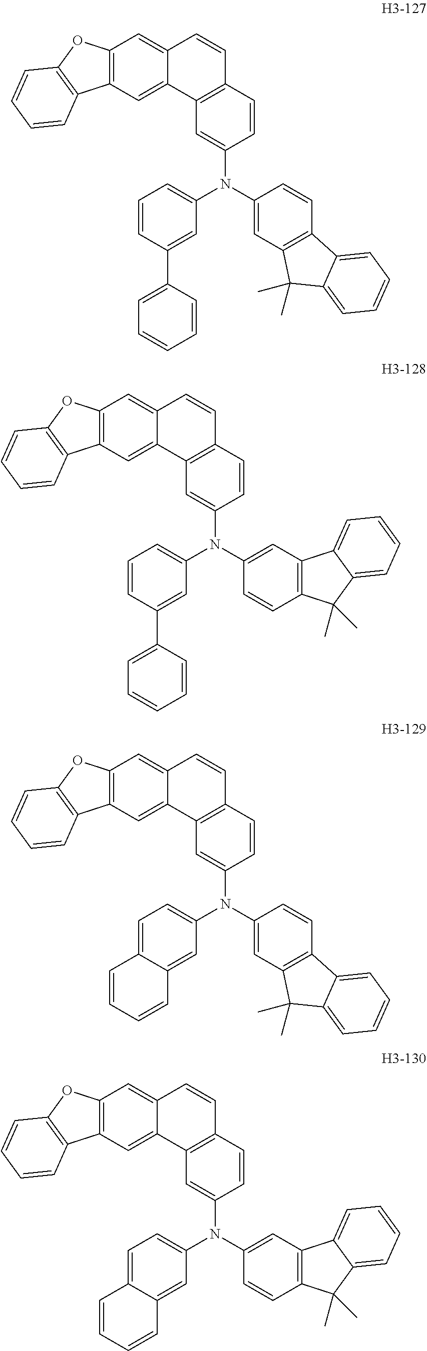 [SMILES: CC1(C)c2ccccc2-c2ccc(N(c3cccc(-c4ccccc4)c3)c3ccc4ccc5cc6oc7ccccc7c6cc5c4c3)cc21 ; CC1(C)c2ccccc2-c2cc(N(c3cccc(-c4ccccc4)c3)c3ccc4ccc5cc6oc7ccccc7c6cc5c4c3)ccc21 ; CC1(C)c2ccccc2-c2ccc(N(c3ccc4ccccc4c3)c3ccc4ccc5cc6oc7ccccc7c6cc5c4c3)cc21 ; CC1(C)c2ccccc2-c2cc(N(c3ccc4ccccc4c3)c3ccc4ccc5cc6oc7ccccc7c6cc5c4c3)ccc21]

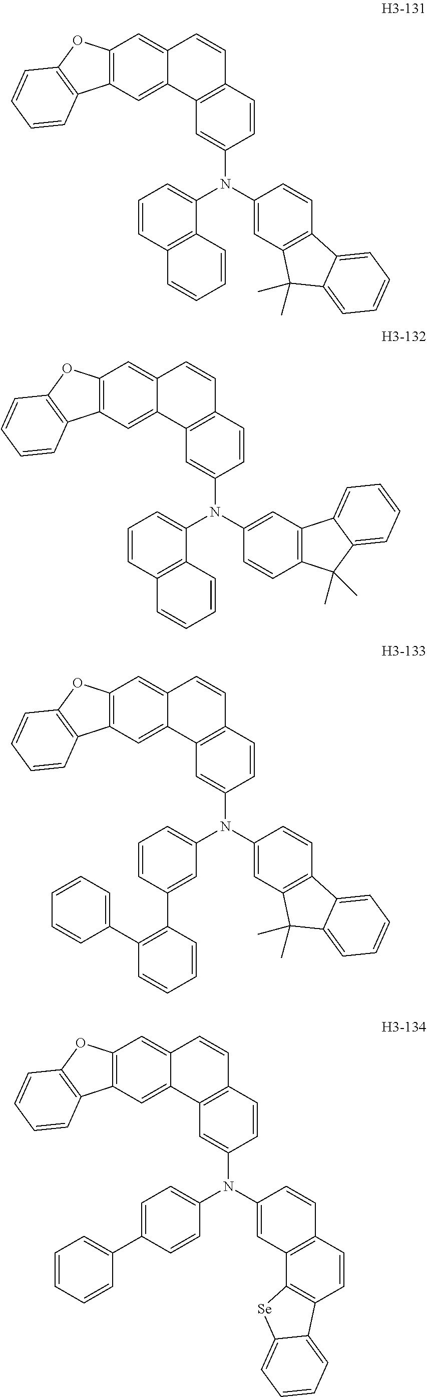 [SMILES: CC1(C)c2ccccc2-c2ccc(N(c3ccc4ccc5cc6oc7ccccc7c6cc5c4c3)c3cccc4ccccc34)cc21 ; CC1(C)c2ccccc2-c2cc(N(c3ccc4ccc5cc6oc7ccccc7c6cc5c4c3)c3cccc4ccccc34)ccc21 ; CC1(C)c2ccccc2-c2ccc(N(c3cccc(-c4ccccc4-c4ccccc4)c3)c3ccc4ccc5cc6oc7ccccc7c6cc5c4c3)cc21 ; c1ccc(-c2ccc(N(c3ccc4ccc5cc6oc7ccccc7c6cc5c4c3)c3ccc4ccc5c6ccccc6[se]c5c4c3)cc2)cc1]

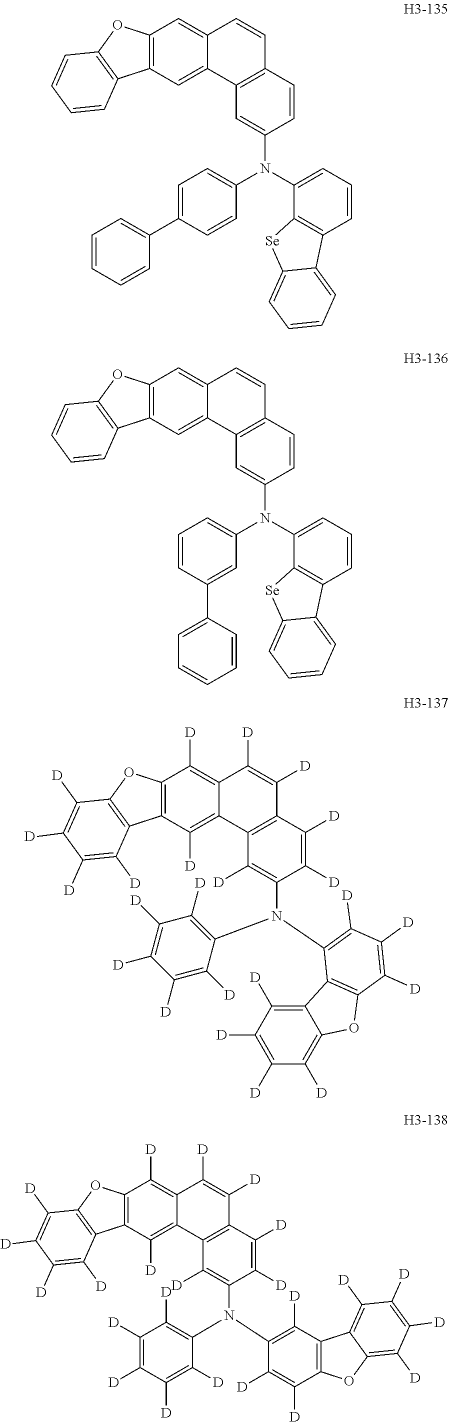

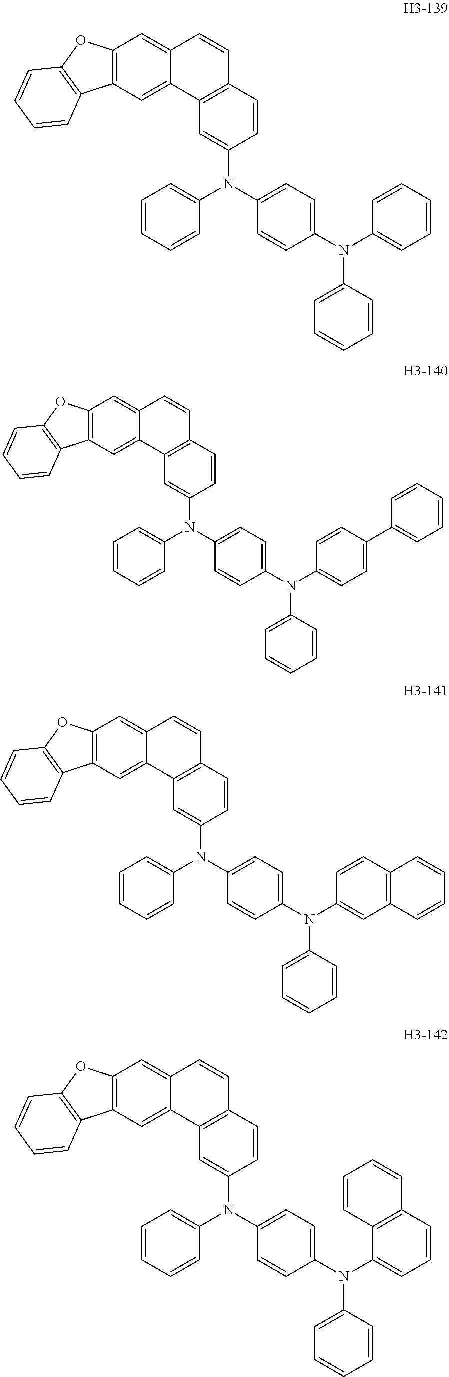 [SMILES: c1ccc(N(c2ccccc2)c2ccc(N(c3ccccc3)c3ccc4ccc5cc6oc7ccccc7c6cc5c4c3)cc2)cc1 ; c1ccc(-c2ccc(N(c3ccccc3)c3ccc(N(c4ccccc4)c4ccc5ccc6cc7oc8ccccc8c7cc6c5c4)cc3)cc2)cc1 ; c1ccc(N(c2ccc(N(c3ccccc3)c3ccc4ccc5cc6oc7ccccc7c6cc5c4c3)cc2)c2ccc3ccccc3c2)cc1 ; c1ccc(N(c2ccc(N(c3ccccc3)c3cccc4ccccc34)cc2)c2ccc3ccc4cc5oc6ccccc6c5cc4c3c2)cc1]

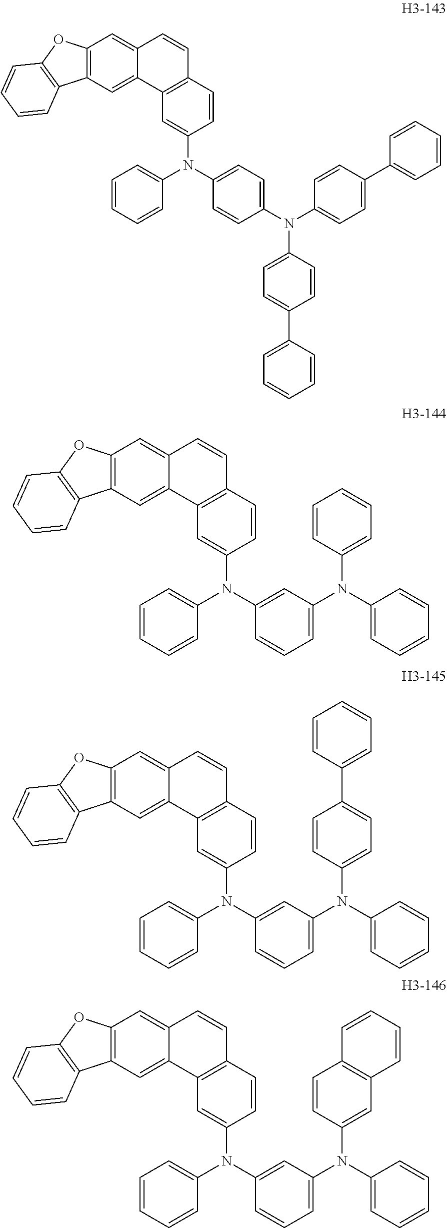 [SMILES: c1ccc(-c2ccc(N(c3ccc(-c4ccccc4)cc3)c3ccc(N(c4ccccc4)c4ccc5ccc6cc7oc8ccccc8c7cc6c5c4)cc3)cc2)cc1 ; c1ccc(N(c2ccccc2)c2cccc(N(c3ccccc3)c3ccc4ccc5cc6oc7ccccc7c6cc5c4c3)c2)cc1 ; c1ccc(-c2ccc(N(c3ccccc3)c3cccc(N(c4ccccc4)c4ccc5ccc6cc7oc8ccccc8c7cc6c5c4)c3)cc2)cc1 ; c1ccc(N(c2cccc(N(c3ccccc3)c3ccc4ccc5cc6oc7ccccc7c6cc5c4c3)c2)c2ccc3ccccc3c2)cc1]

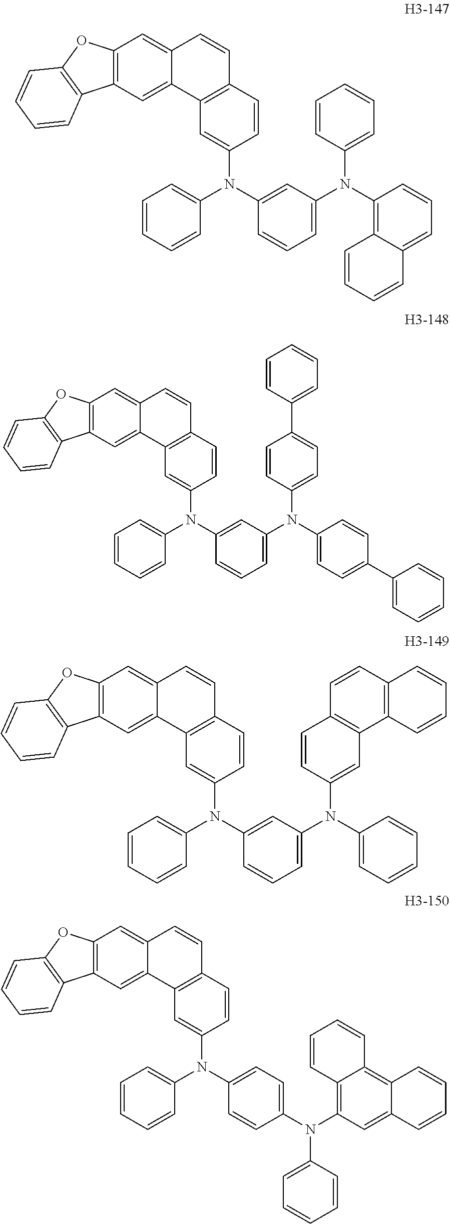 [SMILES: c1ccc(N(c2cccc(N(c3ccccc3)c3cccc4ccccc34)c2)c2ccc3ccc4cc5oc6ccccc6c5cc4c3c2)cc1 ; c1ccc(-c2ccc(N(c3ccc(-c4ccccc4)cc3)c3cccc(N(c4ccccc4)c4ccc5ccc6cc7oc8ccccc8c7cc6c5c4)c3)cc2)cc1 ; c1ccc(N(c2cccc(N(c3ccccc3)c3ccc4ccc5cc6oc7ccccc7c6cc5c4c3)c2)c2ccc3ccc4ccccc4c3c2)cc1 ; c1ccc(N(c2ccc(N(c3ccccc3)c3cc4ccccc4c4ccccc34)cc2)c2ccc3ccc4cc5oc6ccccc6c5cc4c3c2)cc1]

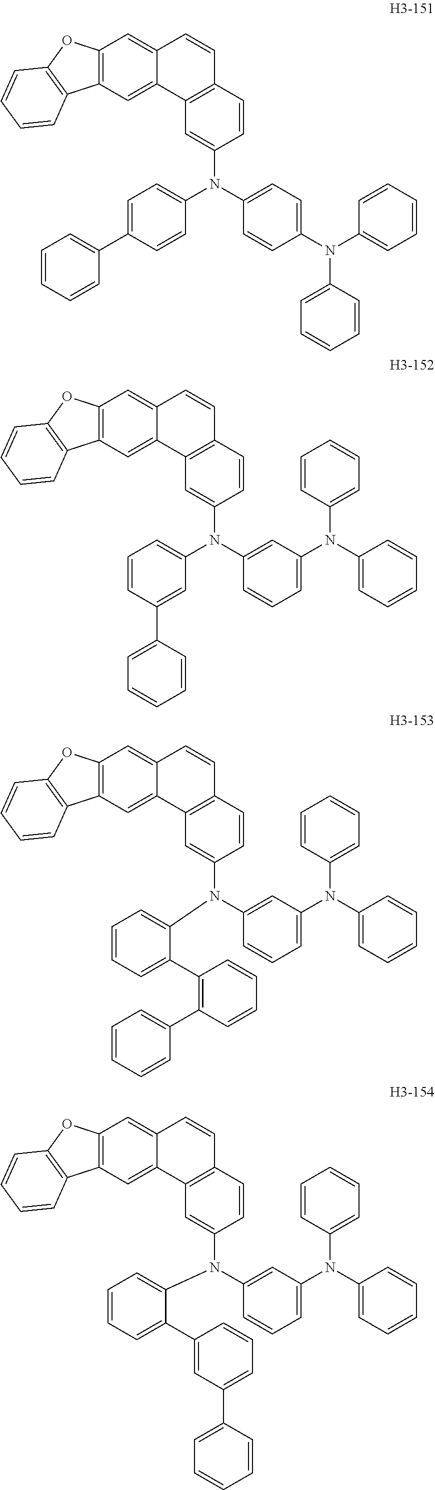

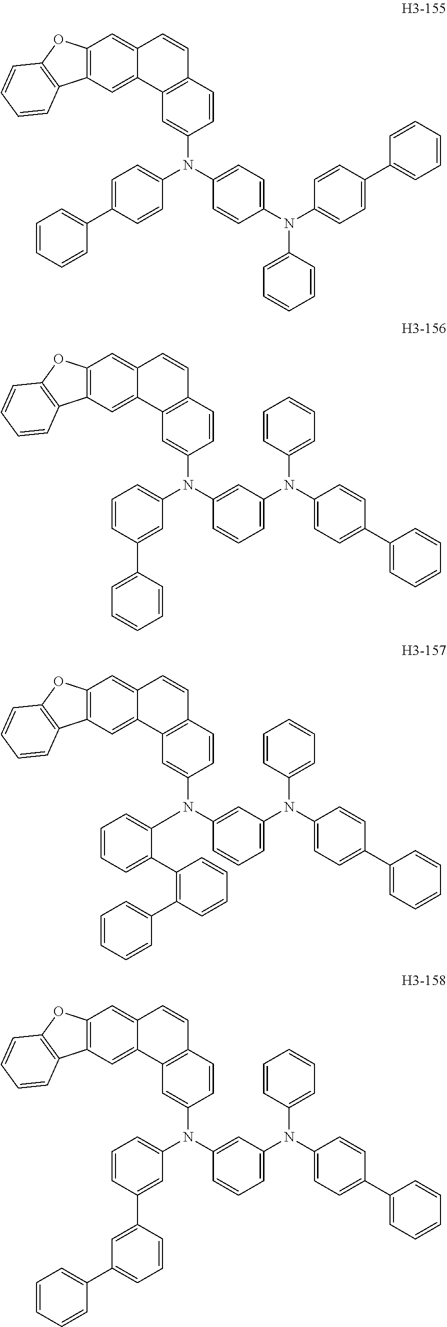 [SMILES: c1ccc(-c2ccc(N(c3ccccc3)c3ccc(N(c4ccc(-c5ccccc5)cc4)c4ccc5ccc6cc7oc8ccccc8c7cc6c5c4)cc3)cc2)cc1 ; c1ccc(-c2ccc(N(c3ccccc3)c3cccc(N(c4cccc(-c5ccccc5)c4)c4ccc5ccc6cc7oc8ccccc8c7cc6c5c4)c3)cc2)cc1 ; c1ccc(-c2ccc(N(c3ccccc3)c3cccc(N(c4ccc5ccc6cc7oc8ccccc8c7cc6c5c4)c4ccccc4-c4ccccc4-c4ccccc4)c3)cc2)cc1 ; c1ccc(-c2ccc(N(c3ccccc3)c3cccc(N(c4cccc(-c5cccc(-c6ccccc6)c5)c4)c4ccc5ccc6cc7oc8ccccc8c7cc6c5c4)c3)cc2)cc1]

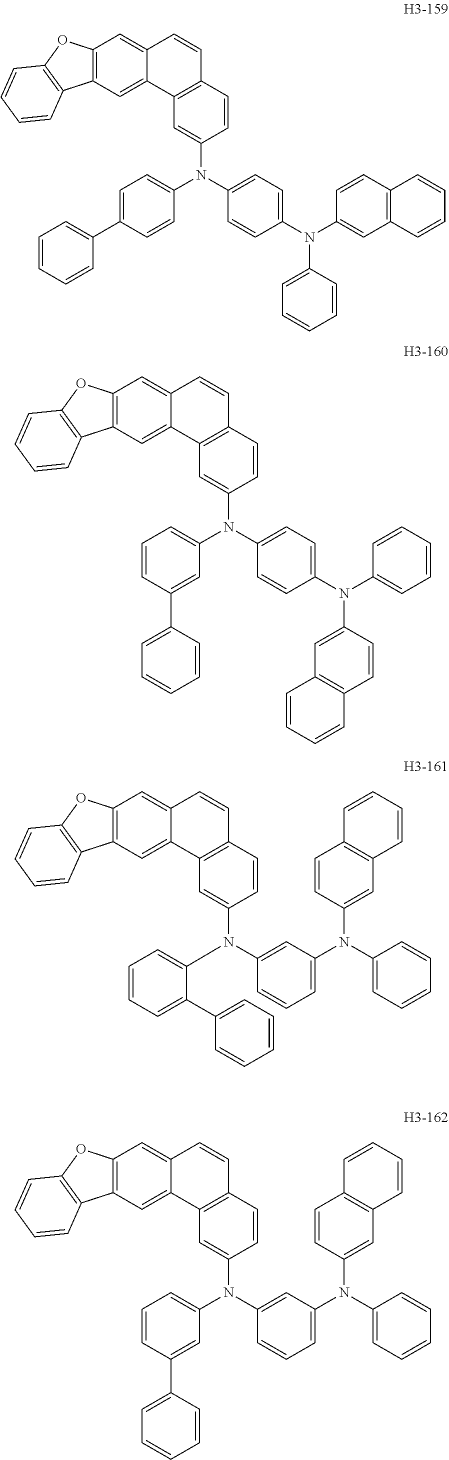 [SMILES: c1ccc(-c2ccc(N(c3ccc(N(c4ccccc4)c4ccc5ccccc5c4)cc3)c3ccc4ccc5cc6oc7ccccc7c6cc5c4c3)cc2)cc1 ; c1ccc(-c2cccc(N(c3ccc(N(c4ccccc4)c4ccc5ccccc5c4)cc3)c3ccc4ccc5cc6oc7ccccc7c6cc5c4c3)c2)cc1 ; c1ccc(-c2ccccc2N(c2cccc(N(c3ccccc3)c3ccc4ccccc4c3)c2)c2ccc3ccc4cc5oc6ccccc6c5cc4c3c2)cc1 ; c1ccc(-c2cccc(N(c3cccc(N(c4ccccc4)c4ccc5ccccc5c4)c3)c3ccc4ccc5cc6oc7ccccc7c6cc5c4c3)c2)cc1]

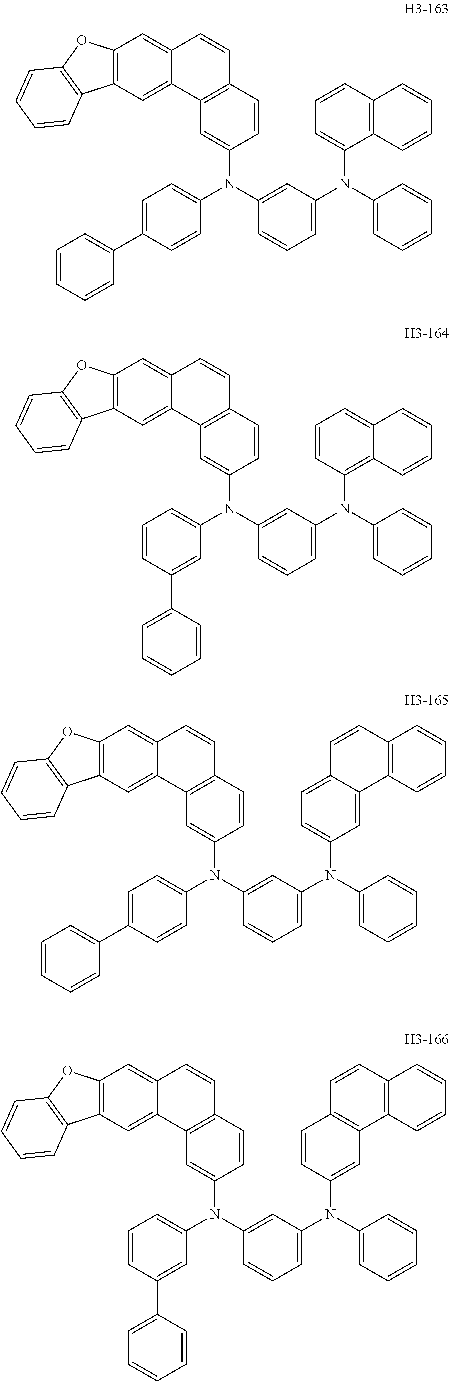 [SMILES: c1ccc(-c2ccc(N(c3cccc(N(c4ccccc4)c4cccc5ccccc45)c3)c3ccc4ccc5cc6oc7ccccc7c6cc5c4c3)cc2)cc1 ; c1ccc(-c2cccc(N(c3cccc(N(c4ccccc4)c4cccc5ccccc45)c3)c3ccc4ccc5cc6oc7ccccc7c6cc5c4c3)c2)cc1 ; c1ccc(-c2ccc(N(c3cccc(N(c4ccccc4)c4ccc5ccc6ccccc6c5c4)c3)c3ccc4ccc5cc6oc7ccccc7c6cc5c4c3)cc2)cc1 ; c1ccc(-c2cccc(N(c3cccc(N(c4ccccc4)c4ccc5ccc6ccccc6c5c4)c3)c3ccc4ccc5cc6oc7ccccc7c6cc5c4c3)c2)cc1]

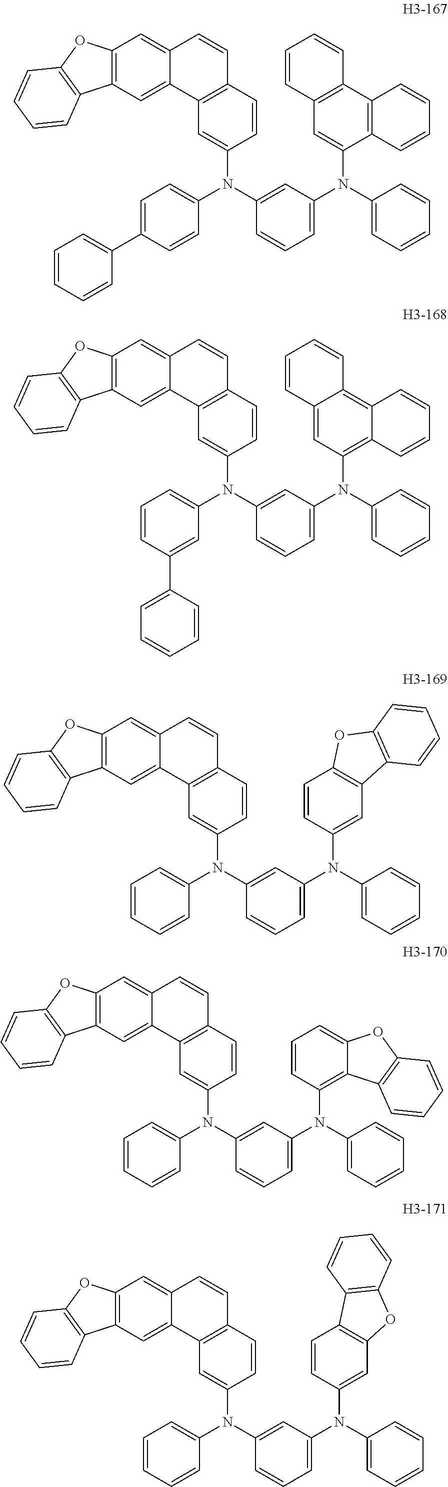

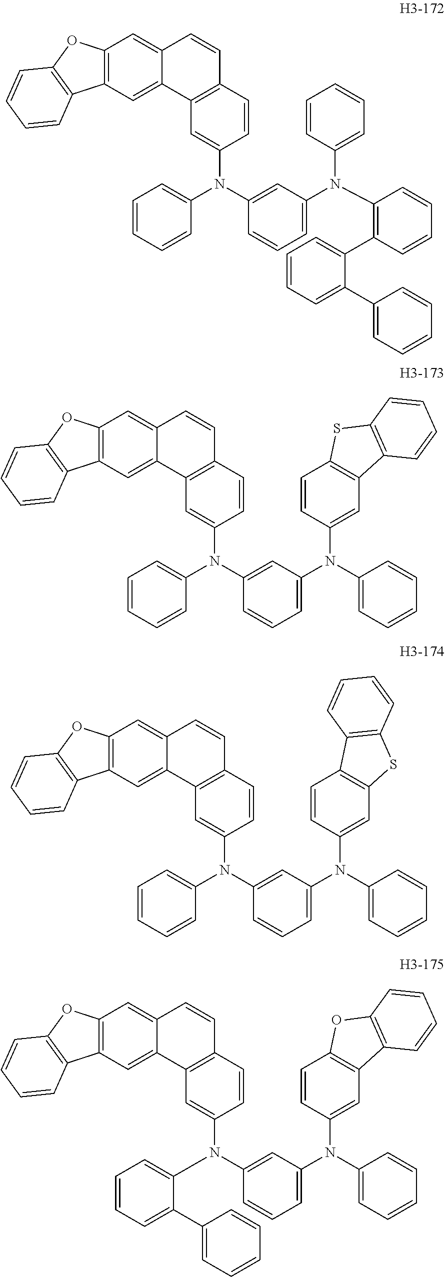

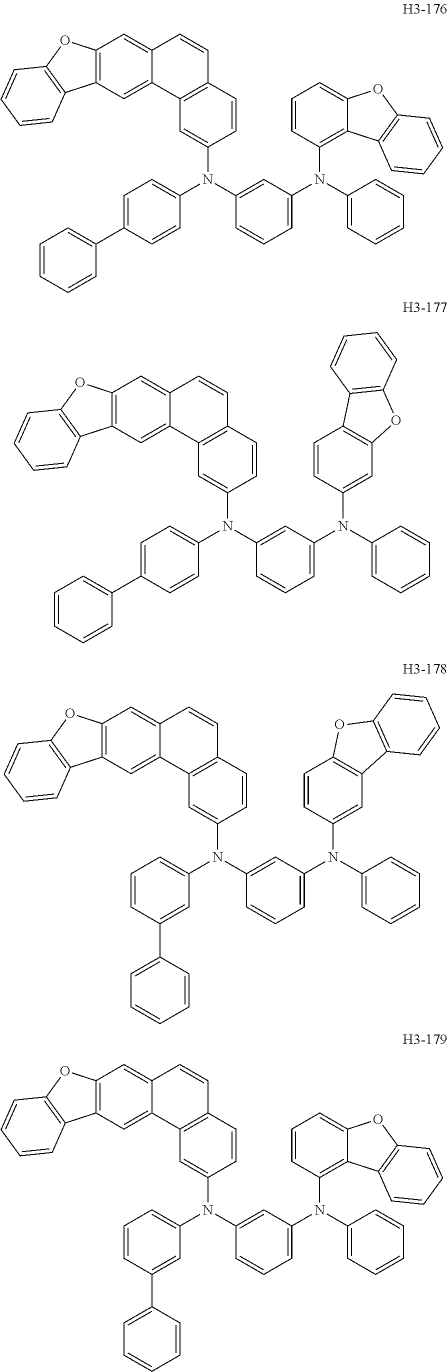

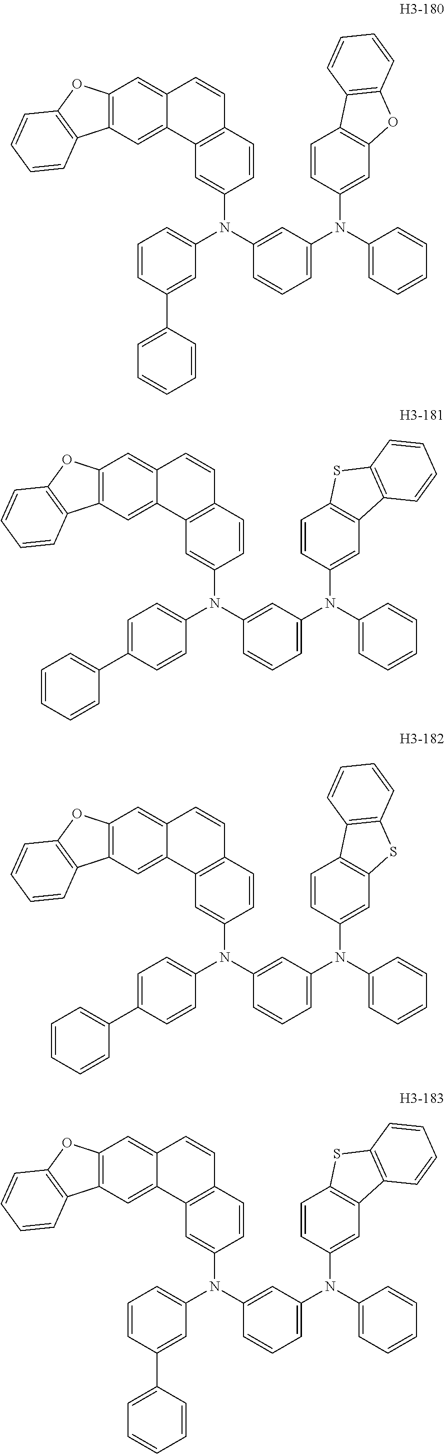 [SMILES: c1ccc(-c2cccc(N(c3cccc(N(c4ccccc4)c4ccc5c(c4)oc4ccccc45)c3)c3ccc4ccc5cc6oc7ccccc7c6cc5c4c3)c2)cc1 ; c1ccc(-c2ccc(N(c3cccc(N(c4ccccc4)c4ccc5sc6ccccc6c5c4)c3)c3ccc4ccc5cc6oc7ccccc7c6cc5c4c3)cc2)cc1 ; c1ccc(-c2ccc(N(c3cccc(N(c4ccccc4)c4ccc5c(c4)sc4ccccc45)c3)c3ccc4ccc5cc6oc7ccccc7c6cc5c4c3)cc2)cc1 ; c1ccc(-c2cccc(N(c3cccc(N(c4ccccc4)c4ccc5sc6ccccc6c5c4)c3)c3ccc4ccc5cc6oc7ccccc7c6cc5c4c3)c2)cc1]

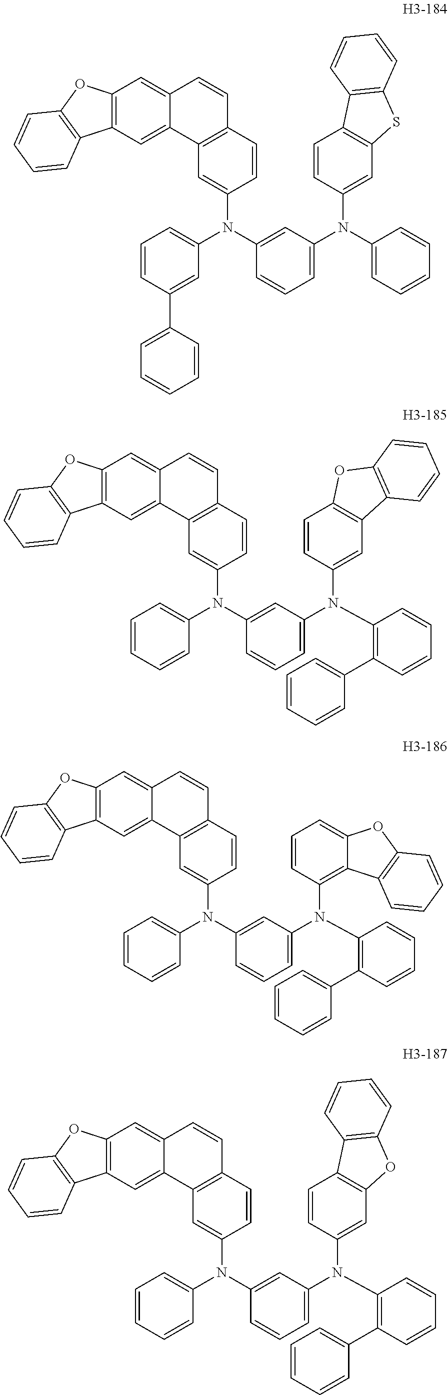 [SMILES: c1ccc(-c2cccc(N(c3cccc(N(c4ccccc4)c4ccc5c(c4)sc4ccccc45)c3)c3ccc4ccc5cc6oc7ccccc7c6cc5c4c3)c2)cc1 ; c1ccc(-c2ccccc2N(c2cccc(N(c3ccccc3)c3ccc4ccc5cc6oc7ccccc7c6cc5c4c3)c2)c2ccc3oc4ccccc4c3c2)cc1 ; c1ccc(-c2ccccc2N(c2cccc(N(c3ccccc3)c3ccc4ccc5cc6oc7ccccc7c6cc5c4c3)c2)c2cccc3oc4ccccc4c23)cc1 ; c1ccc(-c2ccccc2N(c2cccc(N(c3ccccc3)c3ccc4ccc5cc6oc7ccccc7c6cc5c4c3)c2)c2ccc3c(c2)oc2ccccc23)cc1]

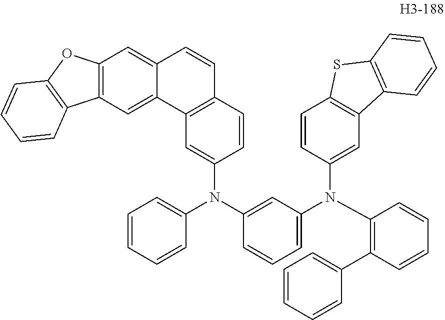 [SMILES: c1ccc(-c2ccccc2N(c2cccc(N(c3ccccc3)c3ccc4ccc5cc6oc7ccccc7c6cc5c4c3)c2)c2ccc3sc4ccccc4c3c2)cc1]

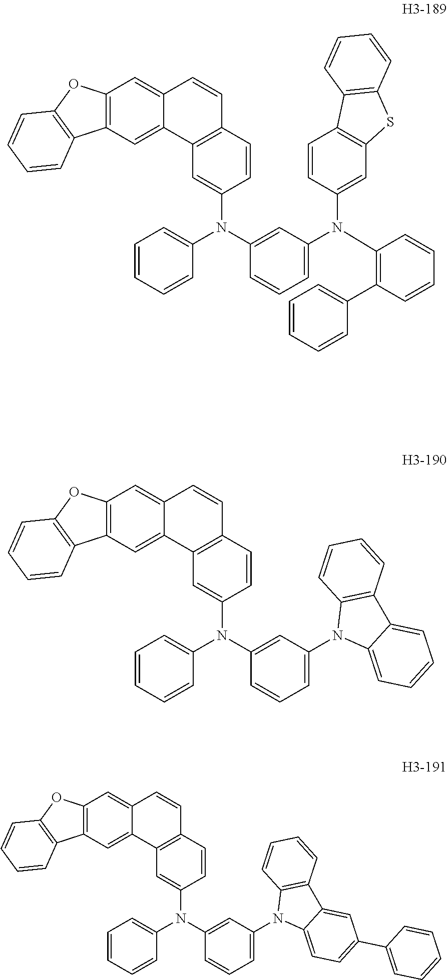 [SMILES: c1ccc(-c2ccccc2N(c2cccc(N(c3ccccc3)c3ccc4ccc5cc6oc7ccccc7c6cc5c4c3)c2)c2ccc3c(c2)sc2ccccc23)cc1 ; c1ccc(N(c2cccc(-n3c4ccccc4c4ccccc43)c2)c2ccc3ccc4cc5oc6ccccc6c5cc4c3c2)cc1 ; c1ccc(-c2ccc3c(c2)c2ccccc2n3-c2cccc(N(c3ccccc3)c3ccc4ccc5cc6oc7ccccc7c6cc5c4c3)c2)cc1]

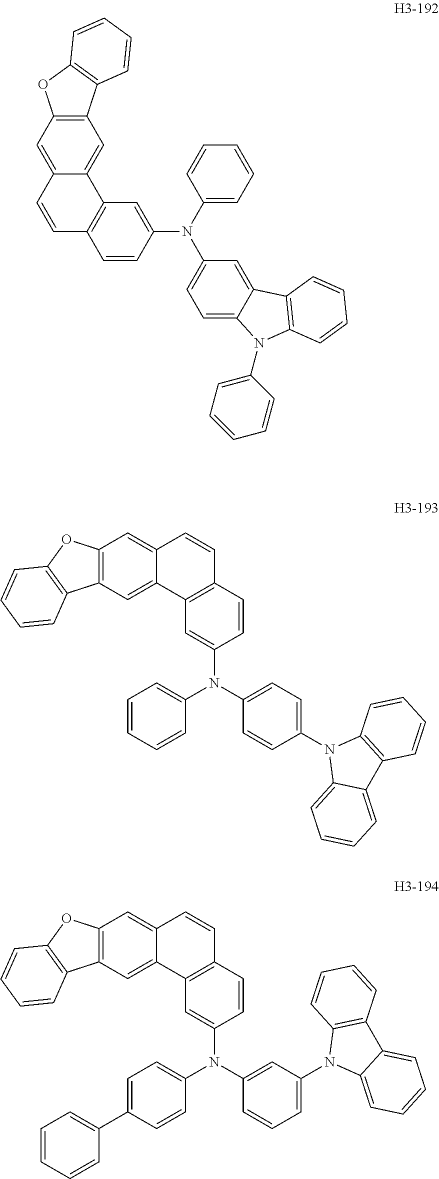 [SMILES: c1ccc(N(c2ccc3ccc4cc5oc6ccccc6c5cc4c3c2)c2ccc3c(c2)c2ccccc2n3-c2ccccc2)cc1 ; c1ccc(N(c2ccc(-n3c4ccccc4c4ccccc43)cc2)c2ccc3ccc4cc5oc6ccccc6c5cc4c3c2)cc1 ; c1ccc(-c2ccc(N(c3cccc(-n4c5ccccc5c5ccccc54)c3)c3ccc4ccc5cc6oc7ccccc7c6cc5c4c3)cc2)cc1]

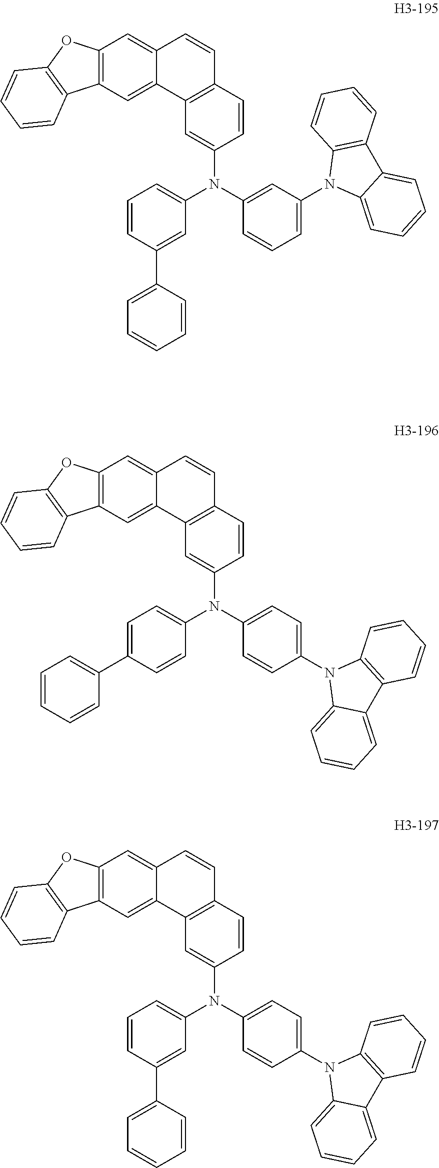 [SMILES: c1ccc(-c2cccc(N(c3cccc(-n4c5ccccc5c5ccccc54)c3)c3ccc4ccc5cc6oc7ccccc7c6cc5c4c3)c2)cc1 ; c1ccc(-c2ccc(N(c3ccc(-n4c5ccccc5c5ccccc54)cc3)c3ccc4ccc5cc6oc7ccccc7c6cc5c4c3)cc2)cc1 ; c1ccc(-c2cccc(N(c3ccc(-n4c5ccccc5c5ccccc54)cc3)c3ccc4ccc5cc6oc7ccccc7c6cc5c4c3)c2)cc1]

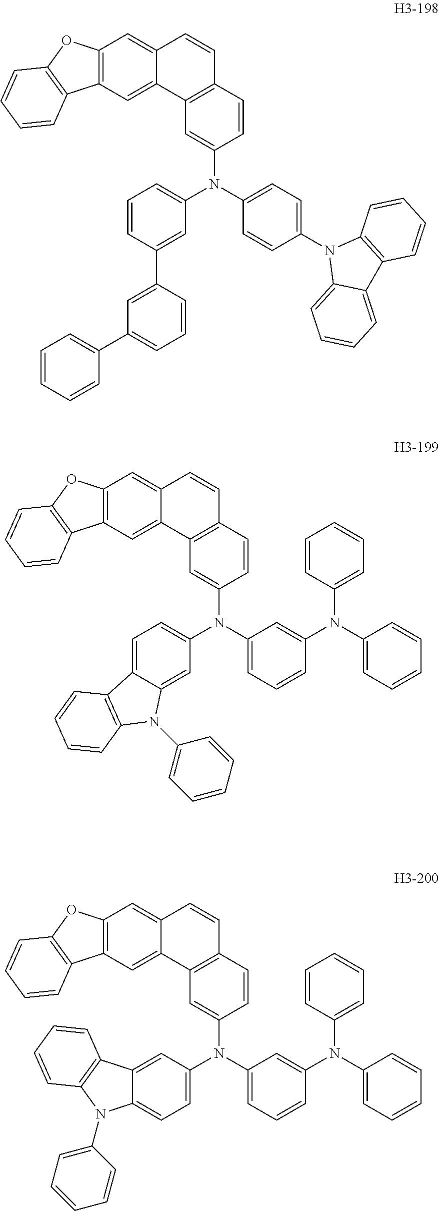 [SMILES: c1ccc(-c2cccc(-c3cccc(N(c4ccc(-n5c6ccccc6c6ccccc65)cc4)c4ccc5ccc6cc7oc8ccccc8c7cc6c5c4)c3)c2)cc1 ; c1ccc(N(c2ccccc2)c2cccc(N(c3ccc4ccc5cc6oc7ccccc7c6cc5c4c3)c3ccc4c5ccccc5n(-c5ccccc5)c4c3)c2)cc1 ; c1ccc(N(c2ccccc2)c2cccc(N(c3ccc4ccc5cc6oc7ccccc7c6cc5c4c3)c3ccc4c(c3)c3ccccc3n4-c3ccccc3)c2)cc1]

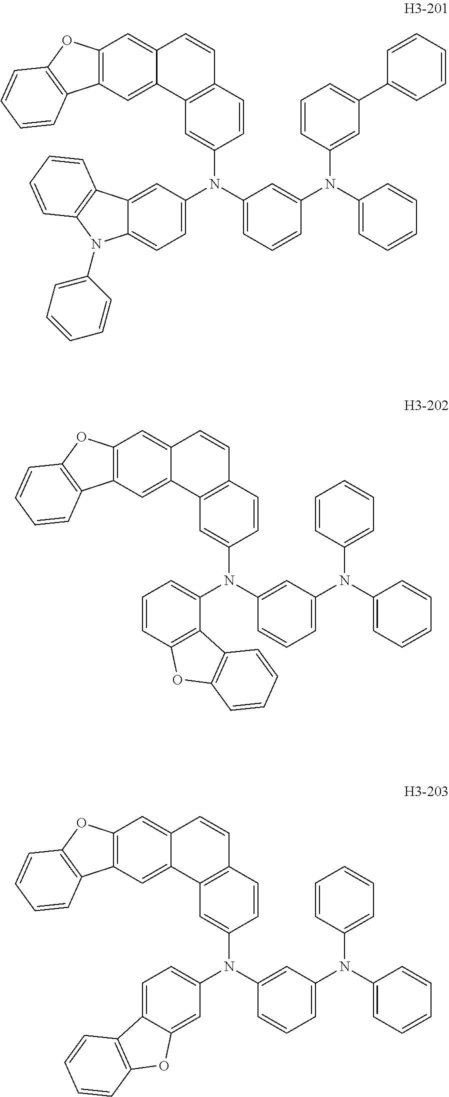 [SMILES: c1ccc(-c2cccc(N(c3ccccc3)c3cccc(N(c4ccc5ccc6cc7oc8ccccc8c7cc6c5c4)c4ccc5c(c4)c4ccccc4n5-c4ccccc4)c3)c2)cc1 ; c1ccc(N(c2ccccc2)c2cccc(N(c3ccc4ccc5cc6oc7ccccc7c6cc5c4c3)c3cccc4oc5ccccc5c34)c2)cc1 ; c1ccc(N(c2ccccc2)c2cccc(N(c3ccc4c(c3)oc3ccccc34)c3ccc4ccc5cc6oc7ccccc7c6cc5c4c3)c2)cc1]

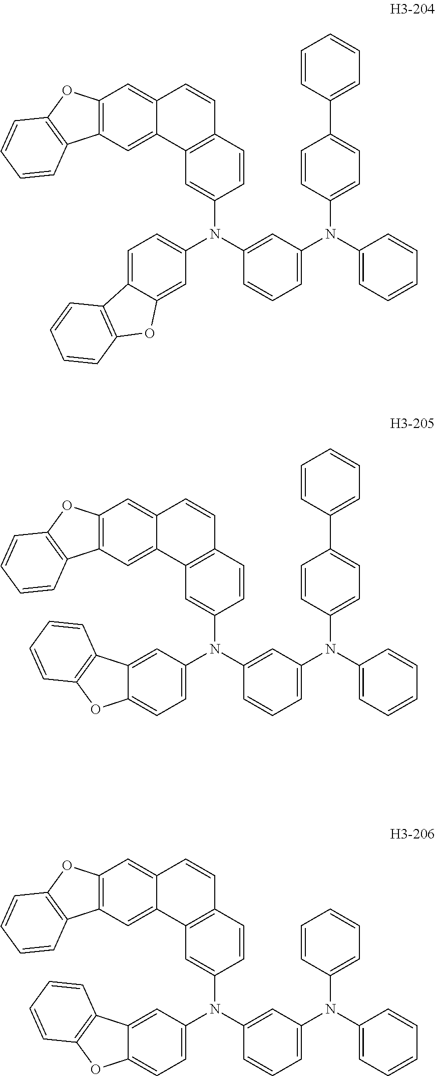 [SMILES: c1ccc(-c2ccc(N(c3ccccc3)c3cccc(N(c4ccc5c(c4)oc4ccccc45)c4ccc5ccc6cc7oc8ccccc8c7cc6c5c4)c3)cc2)cc1 ; c1ccc(-c2ccc(N(c3ccccc3)c3cccc(N(c4ccc5ccc6cc7oc8ccccc8c7cc6c5c4)c4ccc5oc6ccccc6c5c4)c3)cc2)cc1 ; c1ccc(N(c2ccccc2)c2cccc(N(c3ccc4ccc5cc6oc7ccccc7c6cc5c4c3)c3ccc4oc5ccccc5c4c3)c2)cc1]

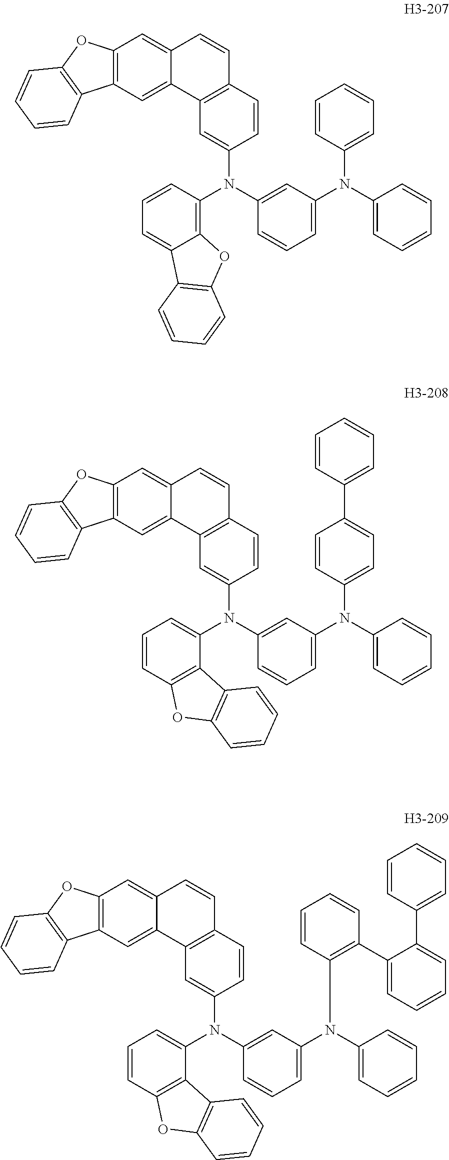 [SMILES: c1ccc(N(c2ccccc2)c2cccc(N(c3ccc4ccc5cc6oc7ccccc7c6cc5c4c3)c3cccc4c3oc3ccccc34)c2)cc1 ; c1ccc(-c2ccc(N(c3ccccc3)c3cccc(N(c4ccc5ccc6cc7oc8ccccc8c7cc6c5c4)c4cccc5oc6ccccc6c45)c3)cc2)cc1 ; c1ccc(-c2ccccc2-c2ccccc2N(c2ccccc2)c2cccc(N(c3ccc4ccc5cc6oc7ccccc7c6cc5c4c3)c3cccc4oc5ccccc5c34)c2)cc1]

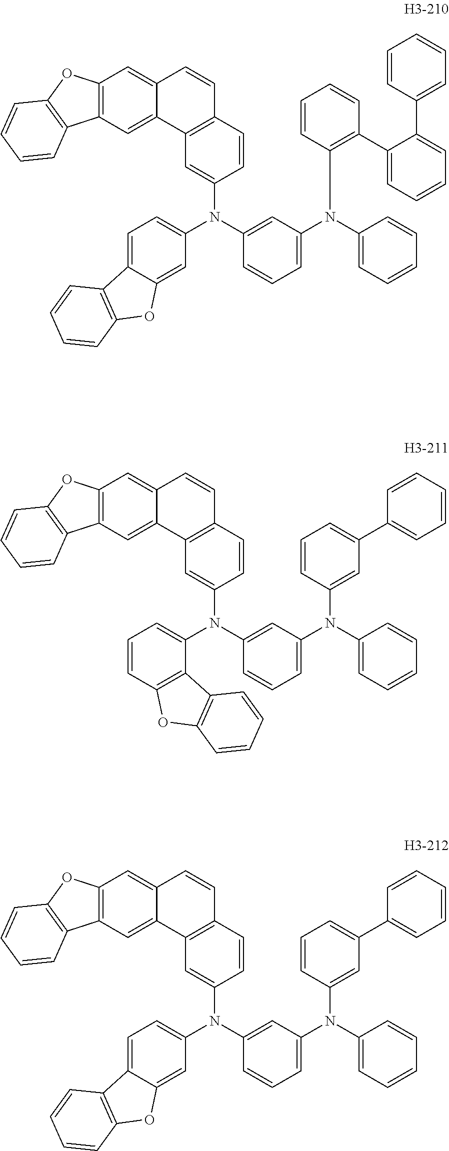 [SMILES: c1ccc(-c2ccccc2-c2ccccc2N(c2ccccc2)c2cccc(N(c3ccc4c(c3)oc3ccccc34)c3ccc4ccc5cc6oc7ccccc7c6cc5c4c3)c2)cc1 ; c1ccc(-c2cccc(N(c3ccccc3)c3cccc(N(c4ccc5ccc6cc7oc8ccccc8c7cc6c5c4)c4cccc5oc6ccccc6c45)c3)c2)cc1 ; c1ccc(-c2cccc(N(c3ccccc3)c3cccc(N(c4ccc5c(c4)oc4ccccc45)c4ccc5ccc6cc7oc8ccccc8c7cc6c5c4)c3)c2)cc1]

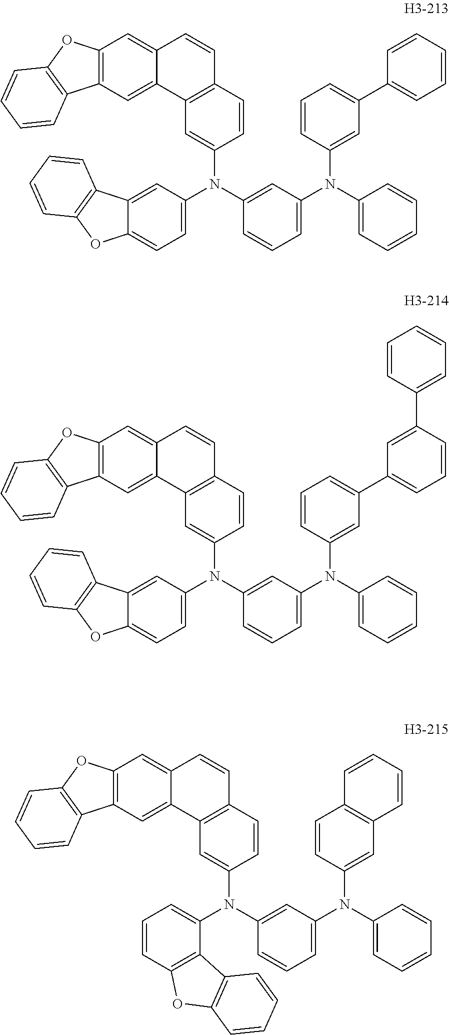 [SMILES: c1ccc(-c2cccc(N(c3ccccc3)c3cccc(N(c4ccc5ccc6cc7oc8ccccc8c7cc6c5c4)c4ccc5oc6ccccc6c5c4)c3)c2)cc1 ; c1ccc(-c2cccc(-c3cccc(N(c4ccccc4)c4cccc(N(c5ccc6ccc7cc8oc9ccccc9c8cc7c6c5)c5ccc6oc7ccccc7c6c5)c4)c3)c2)cc1 ; c1ccc(N(c2cccc(N(c3ccc4ccc5cc6oc7ccccc7c6cc5c4c3)c3cccc4oc5ccccc5c34)c2)c2ccc3ccccc3c2)cc1]

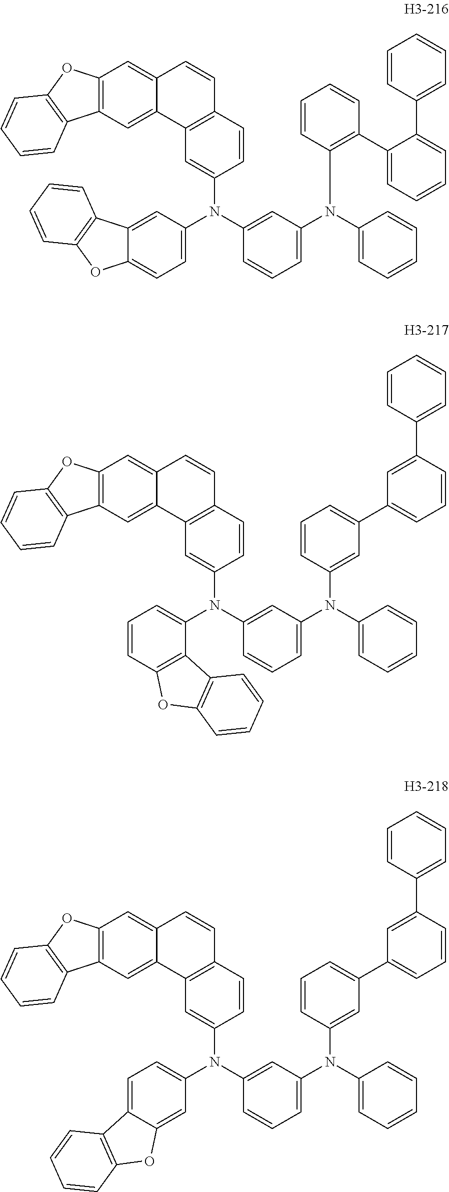 [SMILES: c1ccc(-c2ccccc2-c2ccccc2N(c2ccccc2)c2cccc(N(c3ccc4ccc5cc6oc7ccccc7c6cc5c4c3)c3ccc4oc5ccccc5c4c3)c2)cc1 ; c1ccc(-c2cccc(-c3cccc(N(c4ccccc4)c4cccc(N(c5ccc6ccc7cc8oc9ccccc9c8cc7c6c5)c5cccc6oc7ccccc7c56)c4)c3)c2)cc1 ; c1ccc(-c2cccc(-c3cccc(N(c4ccccc4)c4cccc(N(c5ccc6c(c5)oc5ccccc56)c5ccc6ccc7cc8oc9ccccc9c8cc7c6c5)c4)c3)c2)cc1]

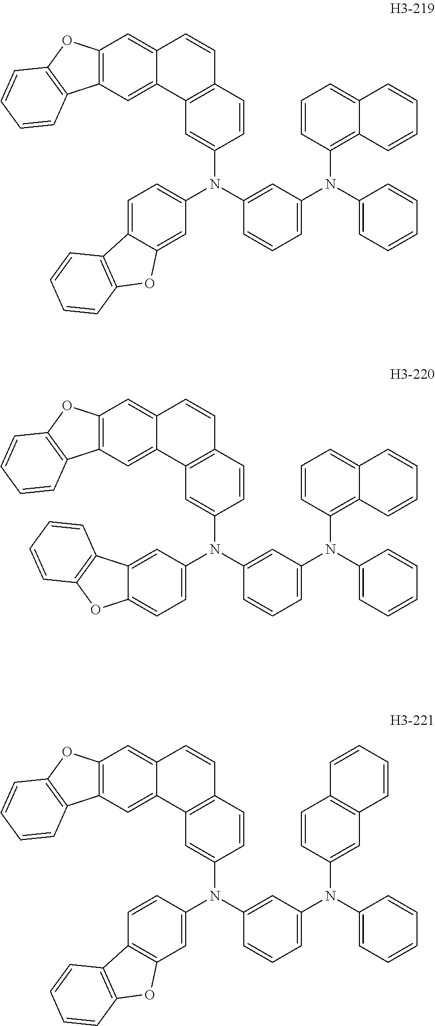 [SMILES: c1ccc(N(c2cccc(N(c3ccc4c(c3)oc3ccccc34)c3ccc4ccc5cc6oc7ccccc7c6cc5c4c3)c2)c2cccc3ccccc23)cc1 ; c1ccc(N(c2cccc(N(c3ccc4ccc5cc6oc7ccccc7c6cc5c4c3)c3ccc4oc5ccccc5c4c3)c2)c2cccc3ccccc23)cc1 ; c1ccc(N(c2cccc(N(c3ccc4c(c3)oc3ccccc34)c3ccc4ccc5cc6oc7ccccc7c6cc5c4c3)c2)c2ccc3ccccc3c2)cc1]

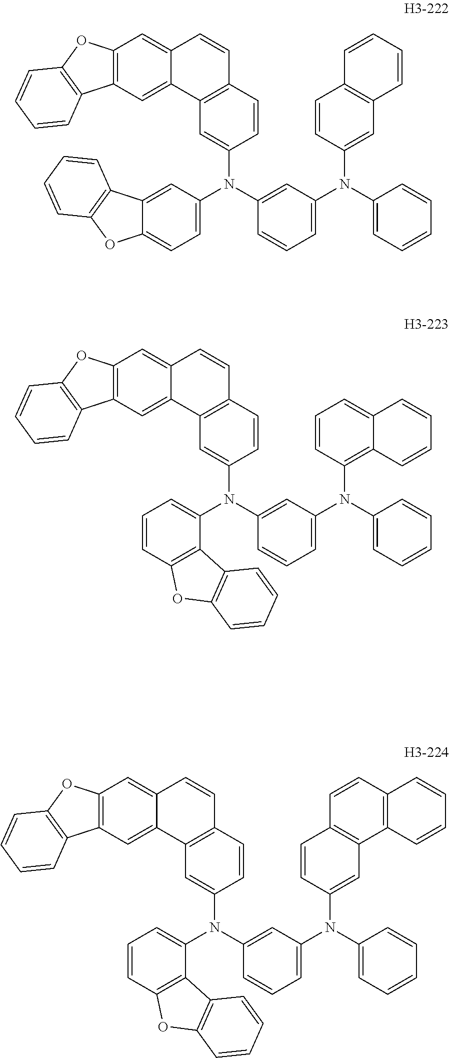 [SMILES: c1ccc(N(c2cccc(N(c3ccc4ccc5cc6oc7ccccc7c6cc5c4c3)c3ccc4oc5ccccc5c4c3)c2)c2ccc3ccccc3c2)cc1 ; c1ccc(N(c2cccc(N(c3ccc4ccc5cc6oc7ccccc7c6cc5c4c3)c3cccc4oc5ccccc5c34)c2)c2cccc3ccccc23)cc1 ; c1ccc(N(c2cccc(N(c3ccc4ccc5cc6oc7ccccc7c6cc5c4c3)c3cccc4oc5ccccc5c34)c2)c2ccc3ccc4ccccc4c3c2)cc1]

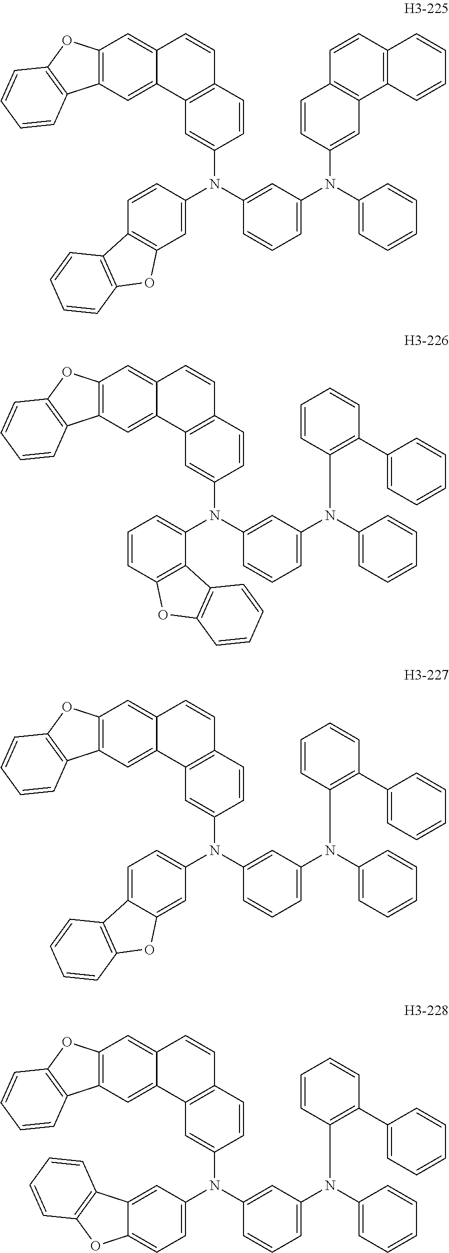 [SMILES: c1ccc(N(c2cccc(N(c3ccc4c(c3)oc3ccccc34)c3ccc4ccc5cc6oc7ccccc7c6cc5c4c3)c2)c2ccc3ccc4ccccc4c3c2)cc1 ; c1ccc(-c2ccccc2N(c2ccccc2)c2cccc(N(c3ccc4ccc5cc6oc7ccccc7c6cc5c4c3)c3cccc4oc5ccccc5c34)c2)cc1 ; c1ccc(-c2ccccc2N(c2ccccc2)c2cccc(N(c3ccc4c(c3)oc3ccccc34)c3ccc4ccc5cc6oc7ccccc7c6cc5c4c3)c2)cc1 ; c1ccc(-c2ccccc2N(c2ccccc2)c2cccc(N(c3ccc4ccc5cc6oc7ccccc7c6cc5c4c3)c3ccc4oc5ccccc5c4c3)c2)cc1]

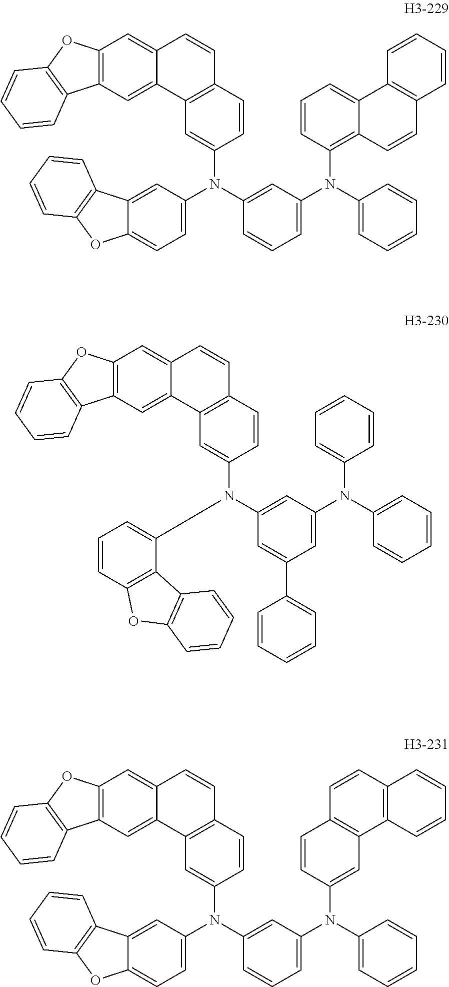 [SMILES: c1ccc(N(c2cccc(N(c3ccc4ccc5cc6oc7ccccc7c6cc5c4c3)c3ccc4oc5ccccc5c4c3)c2)c2cccc3c2ccc2ccccc23)cc1 ; c1ccc(-c2cc(N(c3ccccc3)c3ccccc3)cc(N(c3ccc4ccc5cc6oc7ccccc7c6cc5c4c3)c3cccc4oc5ccccc5c34)c2)cc1 ; c1ccc(N(c2cccc(N(c3ccc4ccc5cc6oc7ccccc7c6cc5c4c3)c3ccc4oc5ccccc5c4c3)c2)c2ccc3ccc4ccccc4c3c2)cc1]

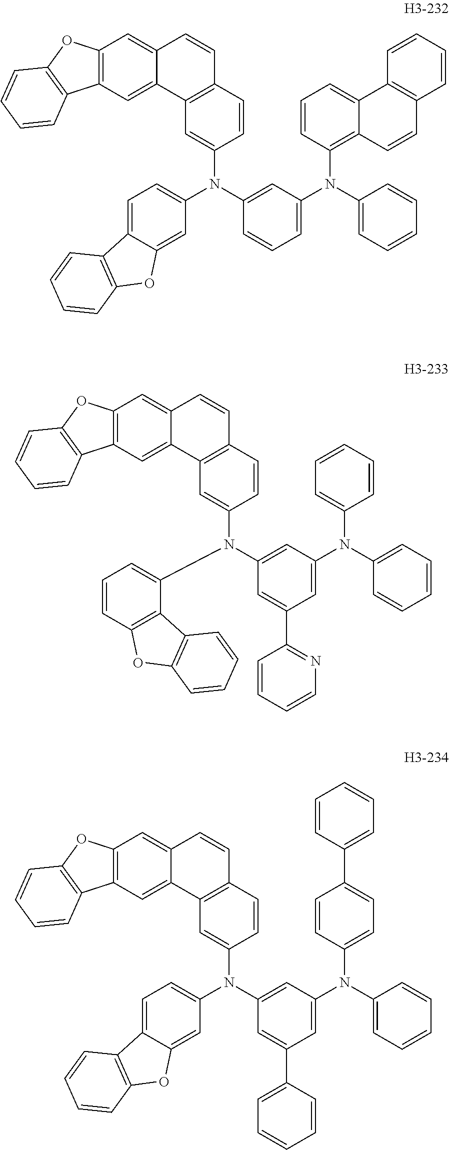 [SMILES: c1ccc(N(c2cccc(N(c3ccc4c(c3)oc3ccccc34)c3ccc4ccc5cc6oc7ccccc7c6cc5c4c3)c2)c2cccc3c2ccc2ccccc23)cc1 ; c1ccc(N(c2ccccc2)c2cc(-c3ccccn3)cc(N(c3ccc4ccc5cc6oc7ccccc7c6cc5c4c3)c3cccc4oc5ccccc5c34)c2)cc1 ; c1ccc(-c2ccc(N(c3ccccc3)c3cc(-c4ccccc4)cc(N(c4ccc5c(c4)oc4ccccc45)c4ccc5ccc6cc7oc8ccccc8c7cc6c5c4)c3)cc2)cc1]

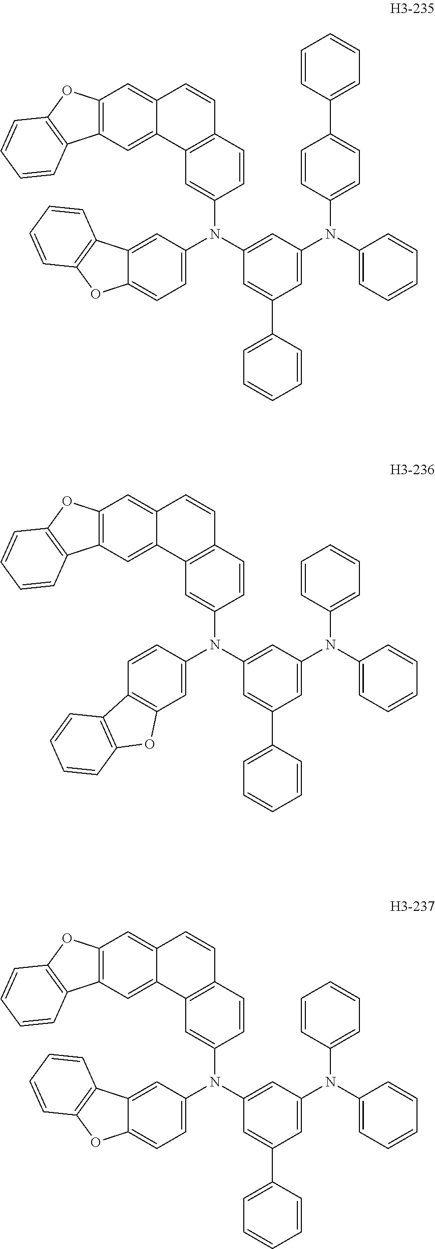 [SMILES: c1ccc(-c2ccc(N(c3ccccc3)c3cc(-c4ccccc4)cc(N(c4ccc5ccc6cc7oc8ccccc8c7cc6c5c4)c4ccc5oc6ccccc6c5c4)c3)cc2)cc1 ; c1ccc(-c2cc(N(c3ccccc3)c3ccccc3)cc(N(c3ccc4c(c3)oc3ccccc34)c3ccc4ccc5cc6oc7ccccc7c6cc5c4c3)c2)cc1 ; c1ccc(-c2cc(N(c3ccccc3)c3ccccc3)cc(N(c3ccc4ccc5cc6oc7ccccc7c6cc5c4c3)c3ccc4oc5ccccc5c4c3)c2)cc1]

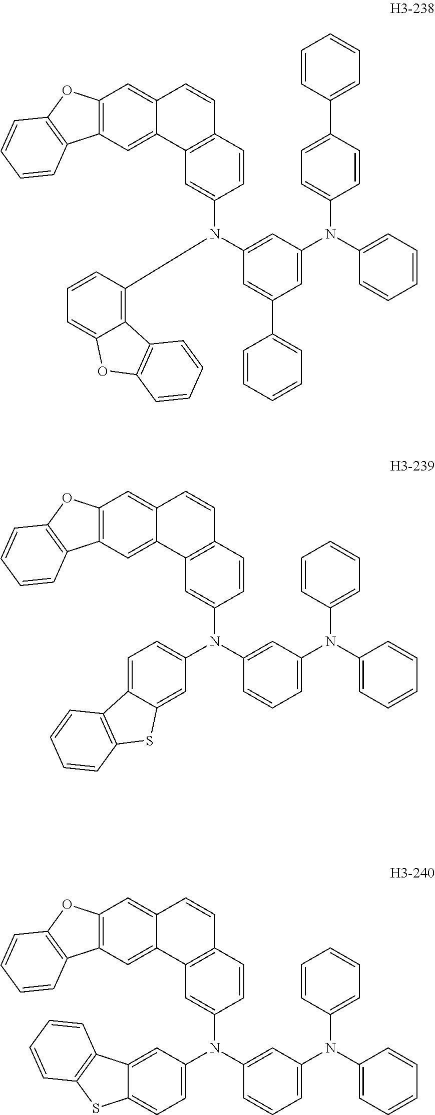 [SMILES: c1ccc(-c2ccc(N(c3ccccc3)c3cc(-c4ccccc4)cc(N(c4ccc5ccc6cc7oc8ccccc8c7cc6c5c4)c4cccc5oc6ccccc6c45)c3)cc2)cc1 ; c1ccc(N(c2ccccc2)c2cccc(N(c3ccc4c(c3)sc3ccccc34)c3ccc4ccc5cc6oc7ccccc7c6cc5c4c3)c2)cc1 ; c1ccc(N(c2ccccc2)c2cccc(N(c3ccc4ccc5cc6oc7ccccc7c6cc5c4c3)c3ccc4sc5ccccc5c4c3)c2)cc1]

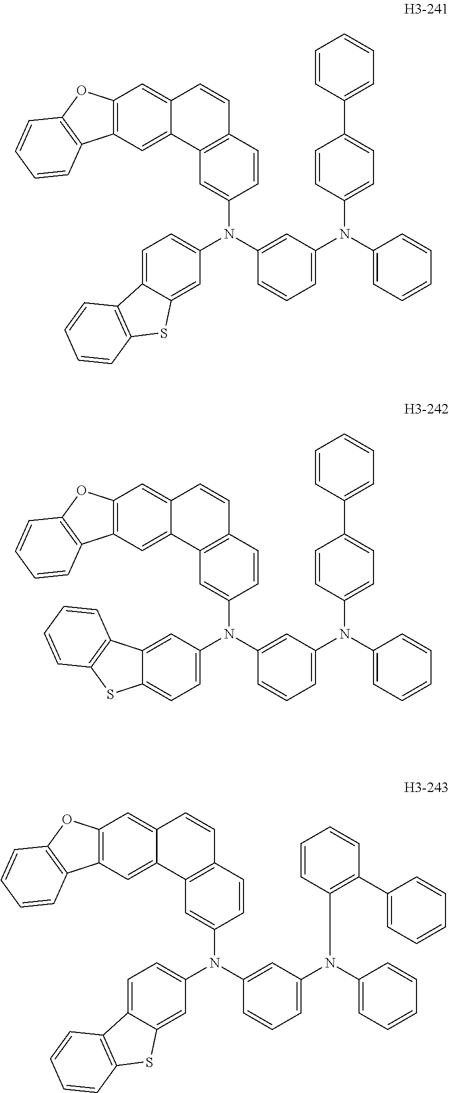 [SMILES: c1ccc(-c2ccc(N(c3ccccc3)c3cccc(N(c4ccc5c(c4)sc4ccccc45)c4ccc5ccc6cc7oc8ccccc8c7cc6c5c4)c3)cc2)cc1 ; c1ccc(-c2ccc(N(c3ccccc3)c3cccc(N(c4ccc5ccc6cc7oc8ccccc8c7cc6c5c4)c4ccc5sc6ccccc6c5c4)c3)cc2)cc1 ; c1ccc(-c2ccccc2N(c2ccccc2)c2cccc(N(c3ccc4c(c3)sc3ccccc34)c3ccc4ccc5cc6oc7ccccc7c6cc5c4c3)c2)cc1]

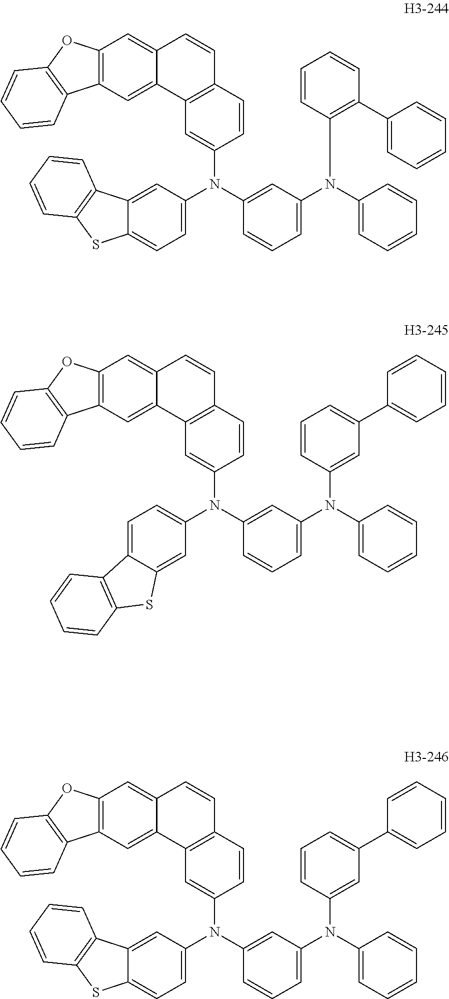 [SMILES: c1ccc(-c2ccccc2N(c2ccccc2)c2cccc(N(c3ccc4ccc5cc6oc7ccccc7c6cc5c4c3)c3ccc4sc5ccccc5c4c3)c2)cc1 ; c1ccc(-c2cccc(N(c3ccccc3)c3cccc(N(c4ccc5c(c4)sc4ccccc45)c4ccc5ccc6cc7oc8ccccc8c7cc6c5c4)c3)c2)cc1 ; c1ccc(-c2cccc(N(c3ccccc3)c3cccc(N(c4ccc5ccc6cc7oc8ccccc8c7cc6c5c4)c4ccc5sc6ccccc6c5c4)c3)c2)cc1]

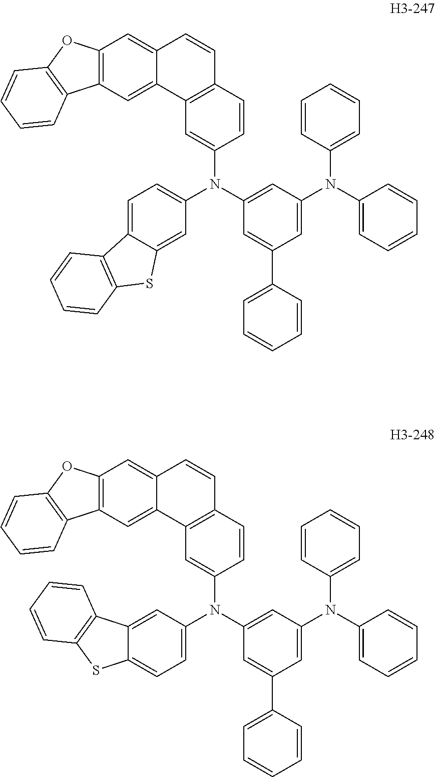 [SMILES: c1ccc(-c2cc(N(c3ccccc3)c3ccccc3)cc(N(c3ccc4c(c3)sc3ccccc34)c3ccc4ccc5cc6oc7ccccc7c6cc5c4c3)c2)cc1 ; c1ccc(-c2cc(N(c3ccccc3)c3ccccc3)cc(N(c3ccc4ccc5cc6oc7ccccc7c6cc5c4c3)c3ccc4sc5ccccc5c4c3)c2)cc1]

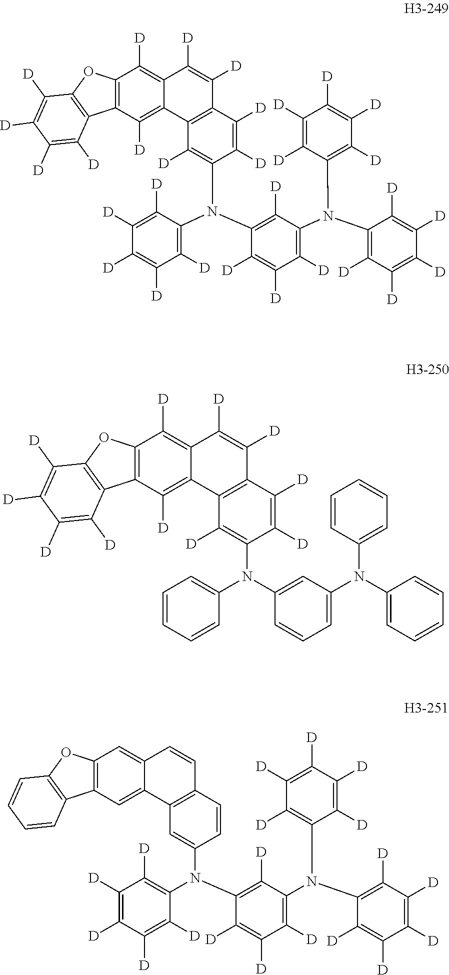

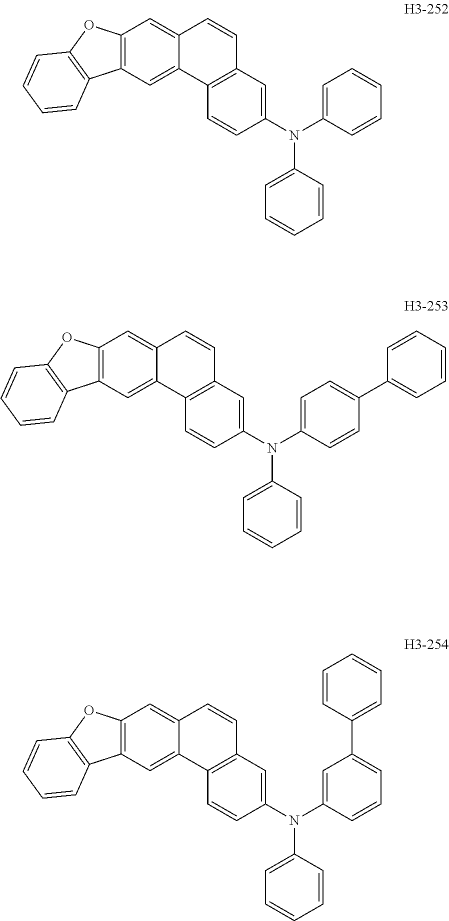 [SMILES: c1ccc(N(c2ccccc2)c2ccc3c(ccc4cc5oc6ccccc6c5cc43)c2)cc1 ; c1ccc(-c2ccc(N(c3ccccc3)c3ccc4c(ccc5cc6oc7ccccc7c6cc54)c3)cc2)cc1 ; c1ccc(-c2cccc(N(c3ccccc3)c3ccc4c(ccc5cc6oc7ccccc7c6cc54)c3)c2)cc1]

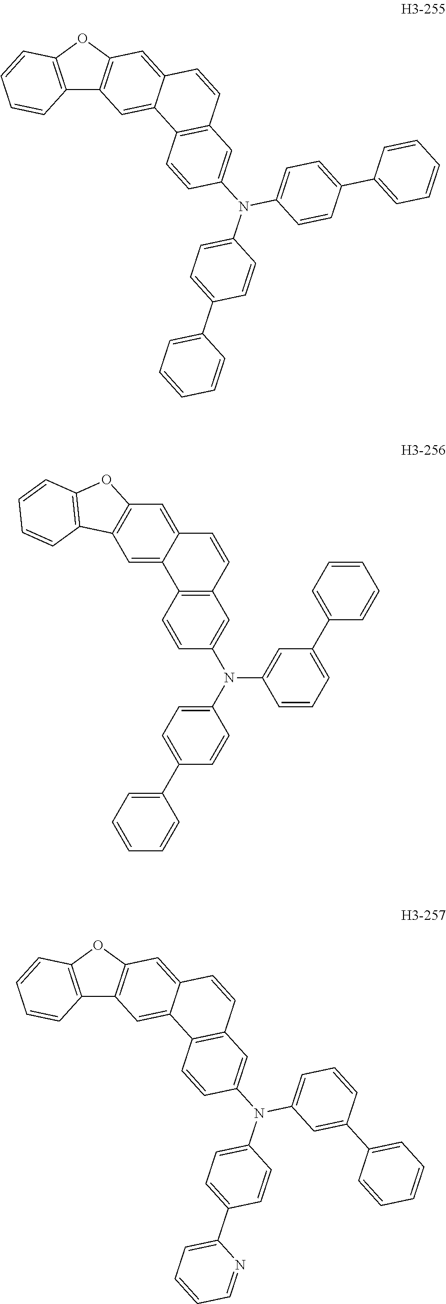 [SMILES: c1ccc(-c2ccc(N(c3ccc(-c4ccccc4)cc3)c3ccc4c(ccc5cc6oc7ccccc7c6cc54)c3)cc2)cc1 ; c1ccc(-c2ccc(N(c3cccc(-c4ccccc4)c3)c3ccc4c(ccc5cc6oc7ccccc7c6cc54)c3)cc2)cc1 ; c1ccc(-c2cccc(N(c3ccc(-c4ccccn4)cc3)c3ccc4c(ccc5cc6oc7ccccc7c6cc54)c3)c2)cc1]

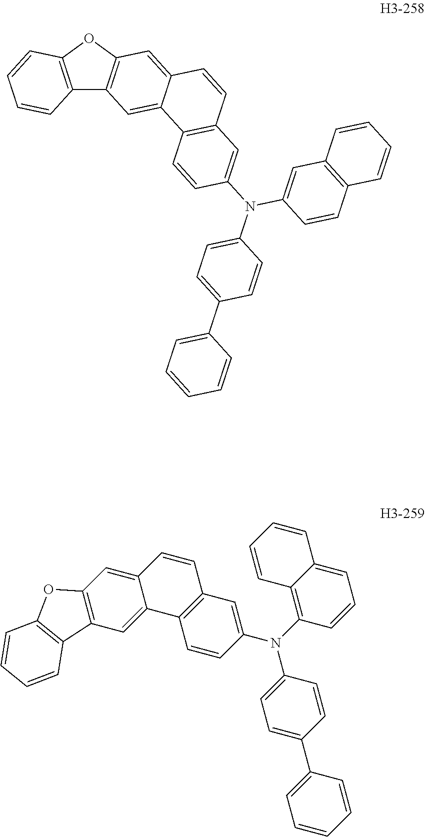 [SMILES: c1ccc(-c2ccc(N(c3ccc4ccccc4c3)c3ccc4c(ccc5cc6oc7ccccc7c6cc54)c3)cc2)cc1 ; c1ccc(-c2ccc(N(c3ccc4c(ccc5cc6oc7ccccc7c6cc54)c3)c3cccc4ccccc34)cc2)cc1]

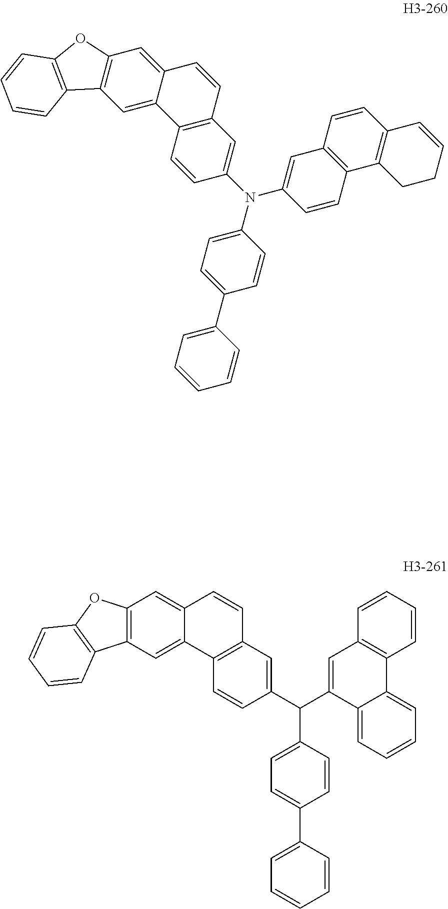 [SMILES: C1=Cc2ccc3cc(N(c4ccc(-c5ccccc5)cc4)c4ccc5c(ccc6cc7oc8ccccc8c7cc65)c4)ccc3c2CC1 ; c1ccc(-c2ccc(C(c3ccc4c(ccc5cc6oc7ccccc7c6cc54)c3)c3cc4ccccc4c4ccccc34)cc2)cc1]

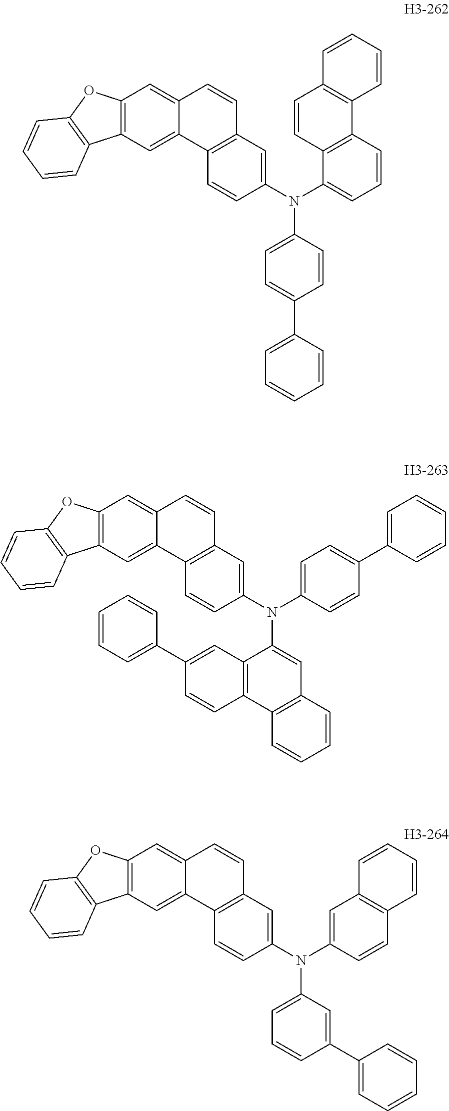 [SMILES: c1ccc(-c2ccc(N(c3ccc4c(ccc5cc6oc7ccccc7c6cc54)c3)c3cccc4c3ccc3ccccc34)cc2)cc1 ; c1ccc(-c2ccc(N(c3ccc4c(ccc5cc6oc7ccccc7c6cc54)c3)c3cc4ccccc4c4ccc(-c5ccccc5)cc34)cc2)cc1 ; c1ccc(-c2cccc(N(c3ccc4ccccc4c3)c3ccc4c(ccc5cc6oc7ccccc7c6cc54)c3)c2)cc1]

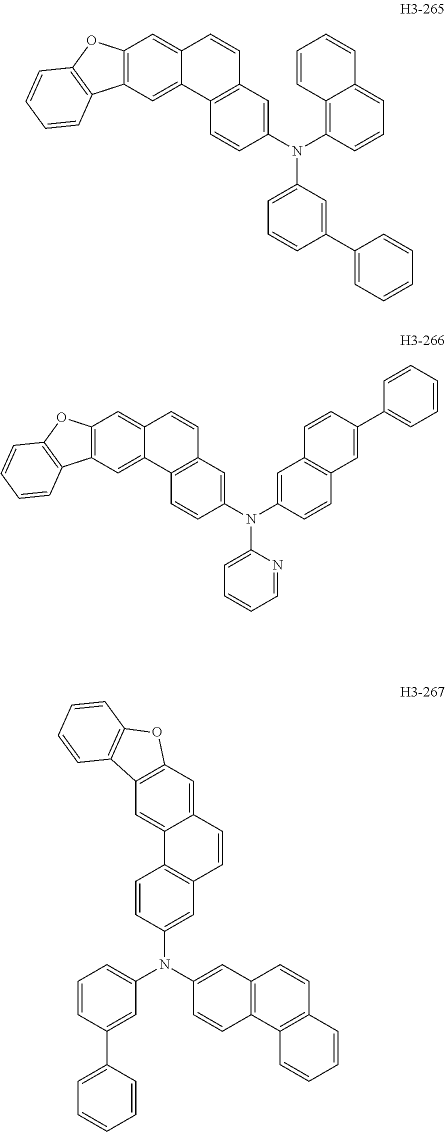 [SMILES: c1ccc(-c2cccc(N(c3ccc4c(ccc5cc6oc7ccccc7c6cc54)c3)c3cccc4ccccc34)c2)cc1 ; c1ccc(-c2ccc3cc(N(c4ccc5c(ccc6cc7oc8ccccc8c7cc65)c4)c4ccccn4)ccc3c2)cc1 ; c1ccc(-c2cccc(N(c3ccc4c(ccc5ccccc54)c3)c3ccc4c(ccc5cc6oc7ccccc7c6cc54)c3)c2)cc1]

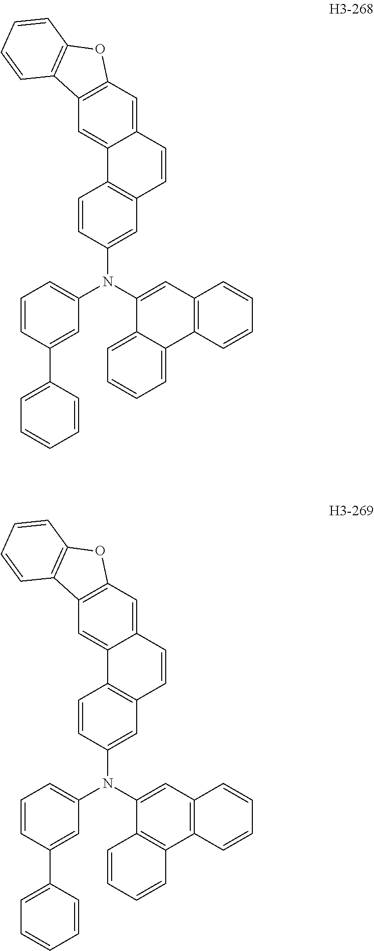 [SMILES: c1ccc(-c2cccc(N(c3ccc4c(ccc5cc6oc7ccccc7c6cc54)c3)c3cc4ccccc4c4ccccc34)c2)cc1 ; c1ccc(-c2cccc(N(c3ccc4c(ccc5cc6oc7ccccc7c6cc54)c3)c3cc4ccccc4c4ccccc34)c2)cc1]

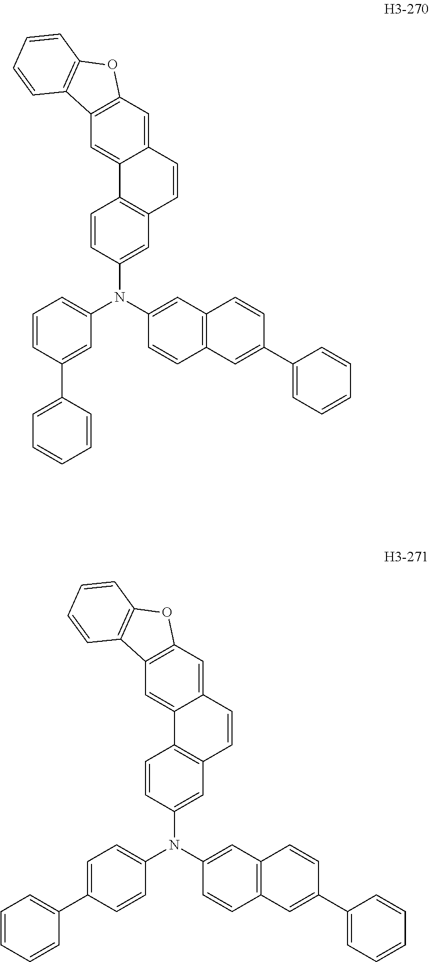 [SMILES: c1ccc(-c2cccc(N(c3ccc4cc(-c5ccccc5)ccc4c3)c3ccc4c(ccc5cc6oc7ccccc7c6cc54)c3)c2)cc1 ; c1ccc(-c2ccc(N(c3ccc4cc(-c5ccccc5)ccc4c3)c3ccc4c(ccc5cc6oc7ccccc7c6cc54)c3)cc2)cc1]

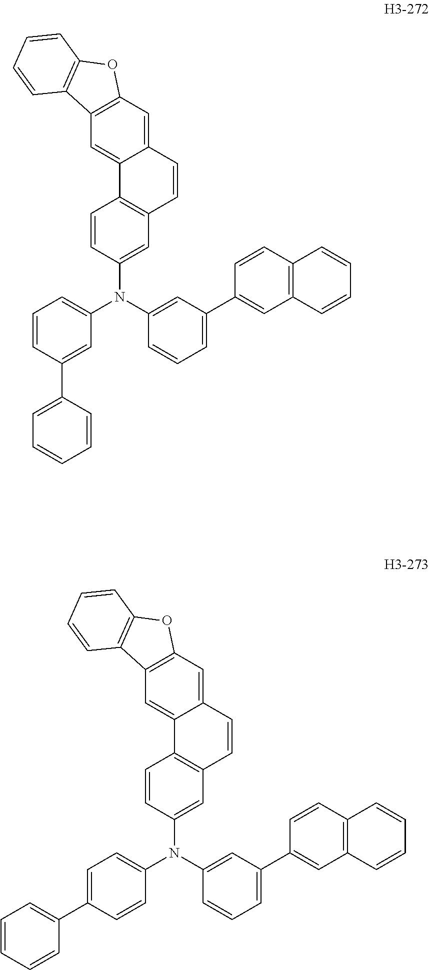 [SMILES: c1ccc(-c2cccc(N(c3cccc(-c4ccc5ccccc5c4)c3)c3ccc4c(ccc5cc6oc7ccccc7c6cc54)c3)c2)cc1 ; c1ccc(-c2ccc(N(c3cccc(-c4ccc5ccccc5c4)c3)c3ccc4c(ccc5cc6oc7ccccc7c6cc54)c3)cc2)cc1]

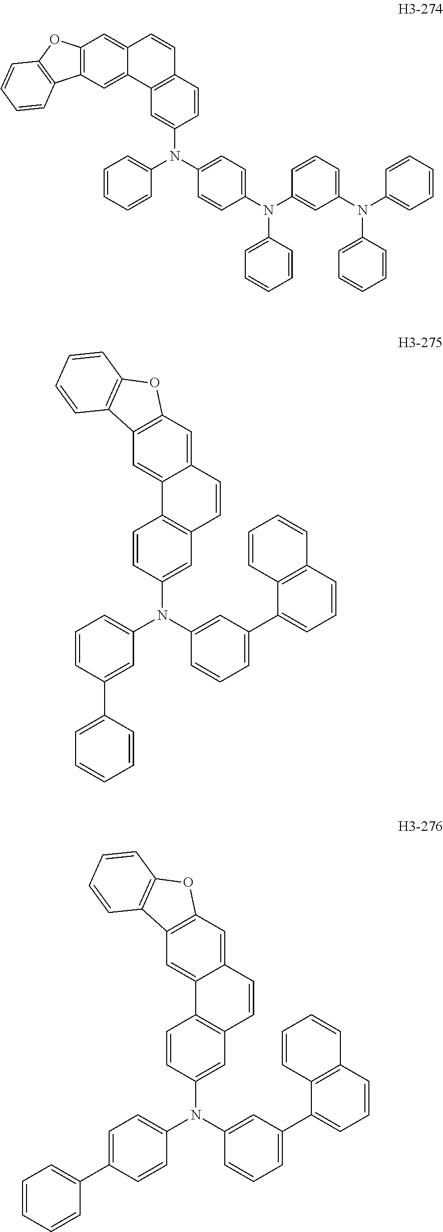 [SMILES: c1ccc(N(c2ccccc2)c2cccc(N(c3ccccc3)c3ccc(N(c4ccccc4)c4ccc5ccc6cc7oc8ccccc8c7cc6c5c4)cc3)c2)cc1 ; c1ccc(-c2cccc(N(c3cccc(-c4cccc5ccccc45)c3)c3ccc4c(ccc5cc6oc7ccccc7c6cc54)c3)c2)cc1 ; c1ccc(-c2ccc(N(c3cccc(-c4cccc5ccccc45)c3)c3ccc4c(ccc5cc6oc7ccccc7c6cc54)c3)cc2)cc1]

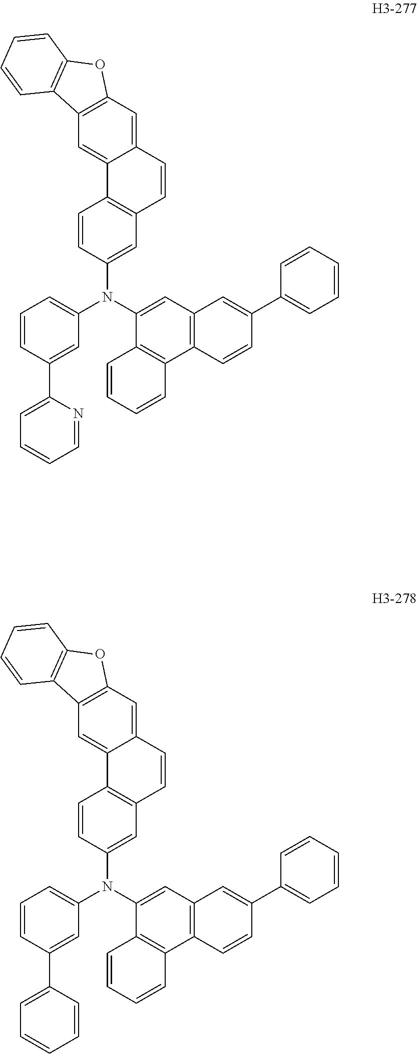 [SMILES: c1ccc(-c2ccc3c(c2)cc(N(c2cccc(-c4ccccn4)c2)c2ccc4c(ccc5cc6oc7ccccc7c6cc54)c2)c2ccccc23)cc1 ; c1ccc(-c2cccc(N(c3ccc4c(ccc5cc6oc7ccccc7c6cc54)c3)c3cc4cc(-c5ccccc5)ccc4c4ccccc34)c2)cc1]

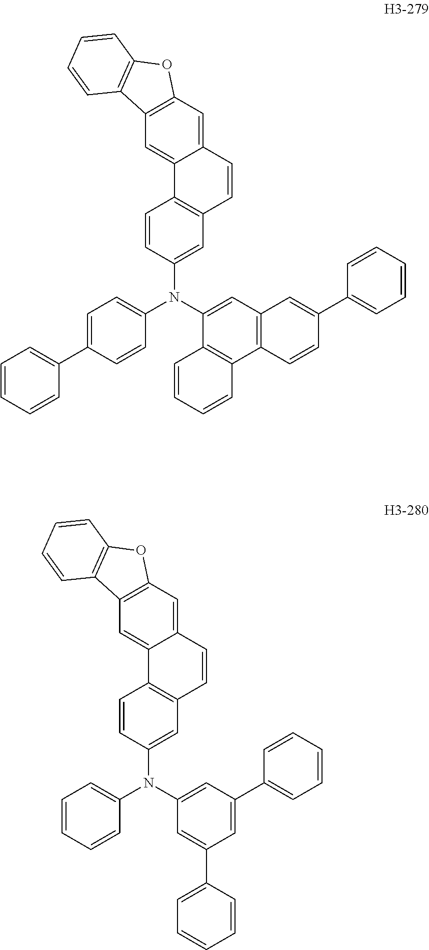 [SMILES: c1ccc(-c2ccc(N(c3ccc4c(ccc5cc6oc7ccccc7c6cc54)c3)c3cc4cc(-c5ccccc5)ccc4c4ccccc34)cc2)cc1 ; c1ccc(-c2cc(-c3ccccc3)cc(N(c3ccccc3)c3ccc4c(ccc5cc6oc7ccccc7c6cc54)c3)c2)cc1]

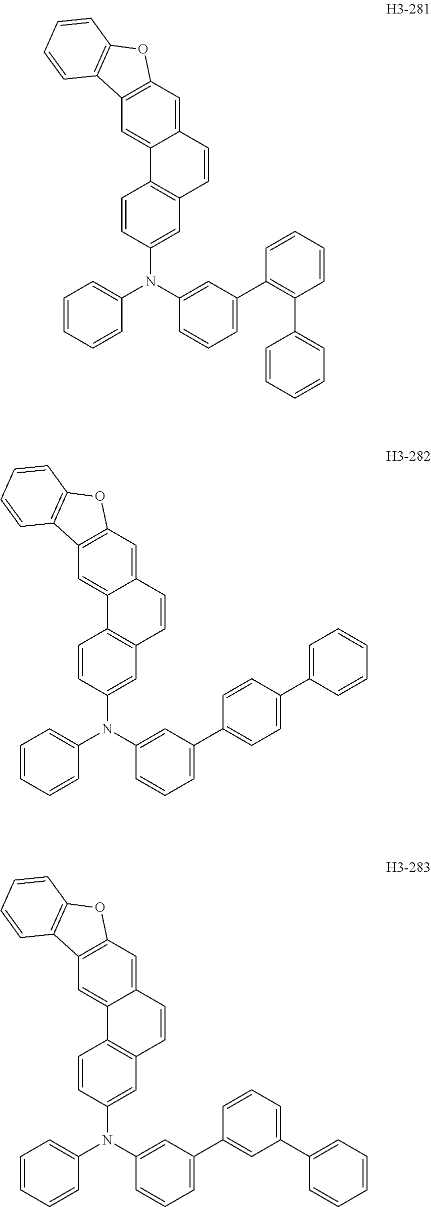 [SMILES: c1ccc(-c2ccccc2-c2cccc(N(c3ccccc3)c3ccc4c(ccc5cc6oc7ccccc7c6cc54)c3)c2)cc1 ; c1ccc(-c2ccc(-c3cccc(N(c4ccccc4)c4ccc5c(ccc6cc7oc8ccccc8c7cc65)c4)c3)cc2)cc1 ; c1ccc(-c2cccc(-c3cccc(N(c4ccccc4)c4ccc5c(ccc6cc7oc8ccccc8c7cc65)c4)c3)c2)cc1]

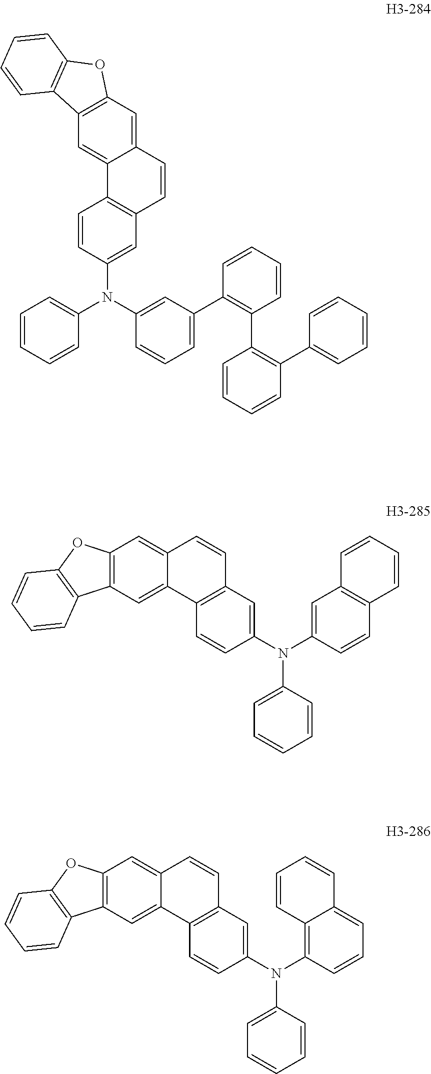 [SMILES: c1ccc(-c2ccccc2-c2ccccc2-c2cccc(N(c3ccccc3)c3ccc4c(ccc5cc6oc7ccccc7c6cc54)c3)c2)cc1 ; c1ccc(N(c2ccc3ccccc3c2)c2ccc3c(ccc4cc5oc6ccccc6c5cc43)c2)cc1 ; c1ccc(N(c2ccc3c(ccc4cc5oc6ccccc6c5cc43)c2)c2cccc3ccccc23)cc1]

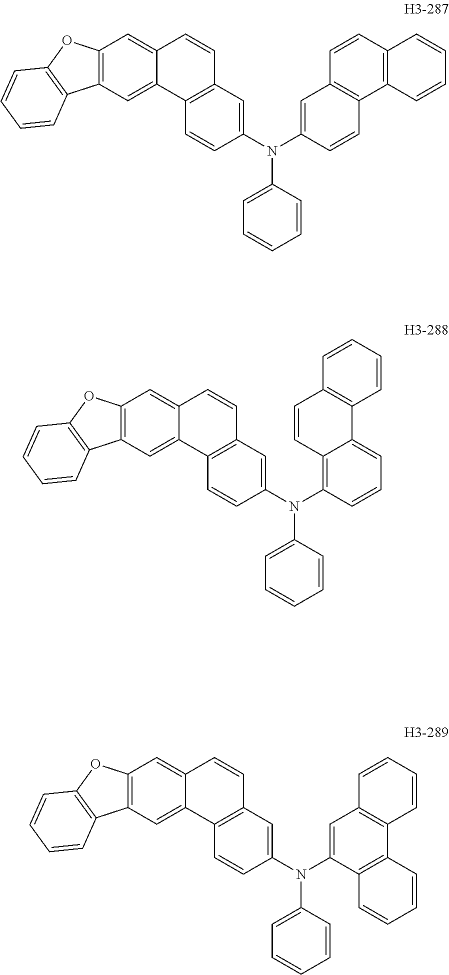 [SMILES: c1ccc(N(c2ccc3c(ccc4ccccc43)c2)c2ccc3c(ccc4cc5oc6ccccc6c5cc43)c2)cc1 ; c1ccc(N(c2ccc3c(ccc4cc5oc6ccccc6c5cc43)c2)c2cccc3c2ccc2ccccc23)cc1 ; c1ccc(N(c2ccc3c(ccc4cc5oc6ccccc6c5cc43)c2)c2cc3ccccc3c3ccccc23)cc1]

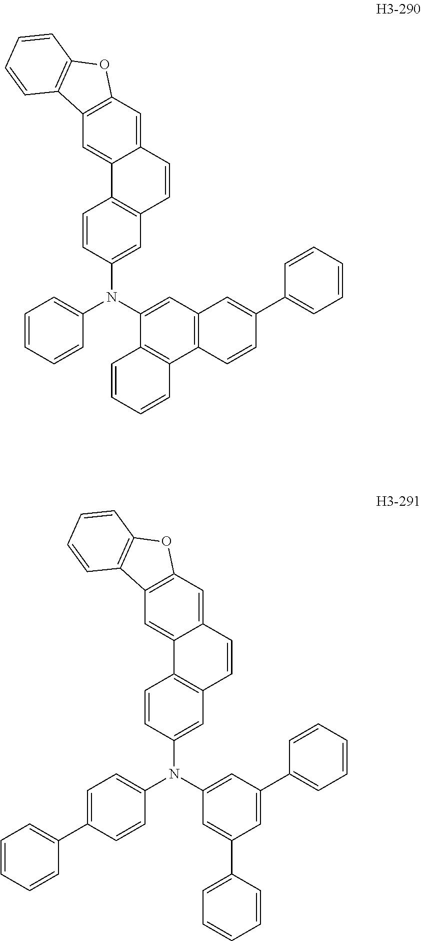 [SMILES: c1ccc(-c2ccc3c(c2)cc(N(c2ccccc2)c2ccc4c(ccc5cc6oc7ccccc7c6cc54)c2)c2ccccc23)cc1 ; c1ccc(-c2ccc(N(c3cc(-c4ccccc4)cc(-c4ccccc4)c3)c3ccc4c(ccc5cc6oc7ccccc7c6cc54)c3)cc2)cc1]

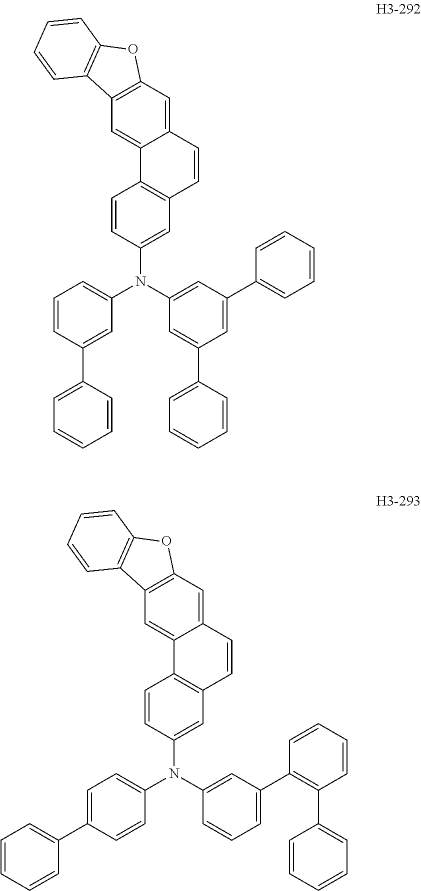 [SMILES: c1ccc(-c2cccc(N(c3cc(-c4ccccc4)cc(-c4ccccc4)c3)c3ccc4c(ccc5cc6oc7ccccc7c6cc54)c3)c2)cc1 ; c1ccc(-c2ccc(N(c3cccc(-c4ccccc4-c4ccccc4)c3)c3ccc4c(ccc5cc6oc7ccccc7c6cc54)c3)cc2)cc1]

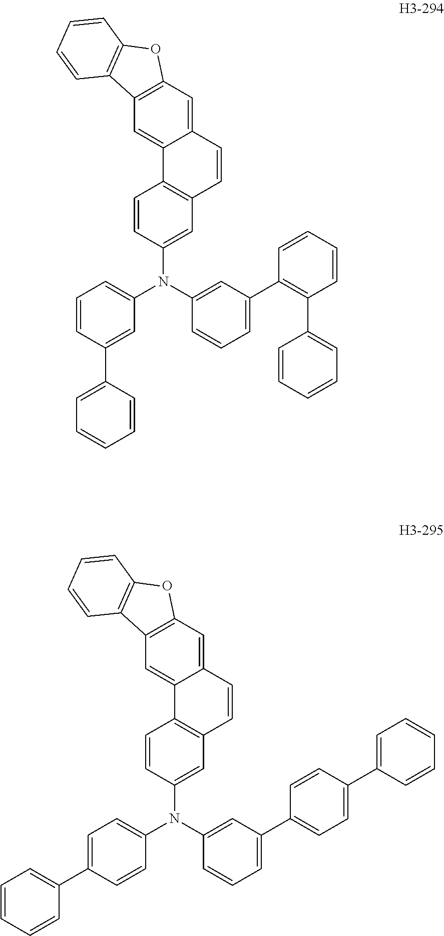 [SMILES: c1ccc(-c2cccc(N(c3cccc(-c4ccccc4-c4ccccc4)c3)c3ccc4c(ccc5cc6oc7ccccc7c6cc54)c3)c2)cc1 ; c1ccc(-c2ccc(-c3cccc(N(c4ccc(-c5ccccc5)cc4)c4ccc5c(ccc6cc7oc8ccccc8c7cc65)c4)c3)cc2)cc1]

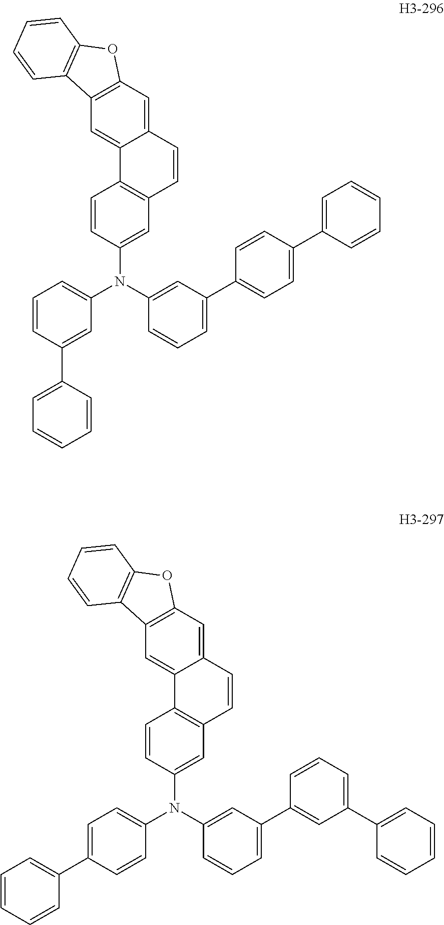 [SMILES: c1ccc(-c2ccc(-c3cccc(N(c4cccc(-c5ccccc5)c4)c4ccc5c(ccc6cc7oc8ccccc8c7cc65)c4)c3)cc2)cc1 ; c1ccc(-c2ccc(N(c3cccc(-c4cccc(-c5ccccc5)c4)c3)c3ccc4c(ccc5cc6oc7ccccc7c6cc54)c3)cc2)cc1]

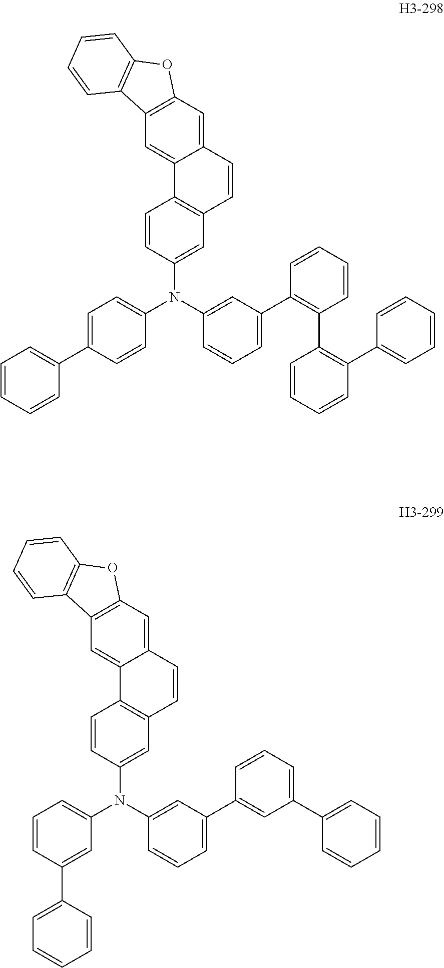 [SMILES: c1ccc(-c2ccc(N(c3cccc(-c4ccccc4-c4ccccc4-c4ccccc4)c3)c3ccc4c(ccc5cc6oc7ccccc7c6cc54)c3)cc2)cc1 ; c1ccc(-c2cccc(-c3cccc(N(c4cccc(-c5ccccc5)c4)c4ccc5c(ccc6cc7oc8ccccc8c7cc65)c4)c3)c2)cc1]

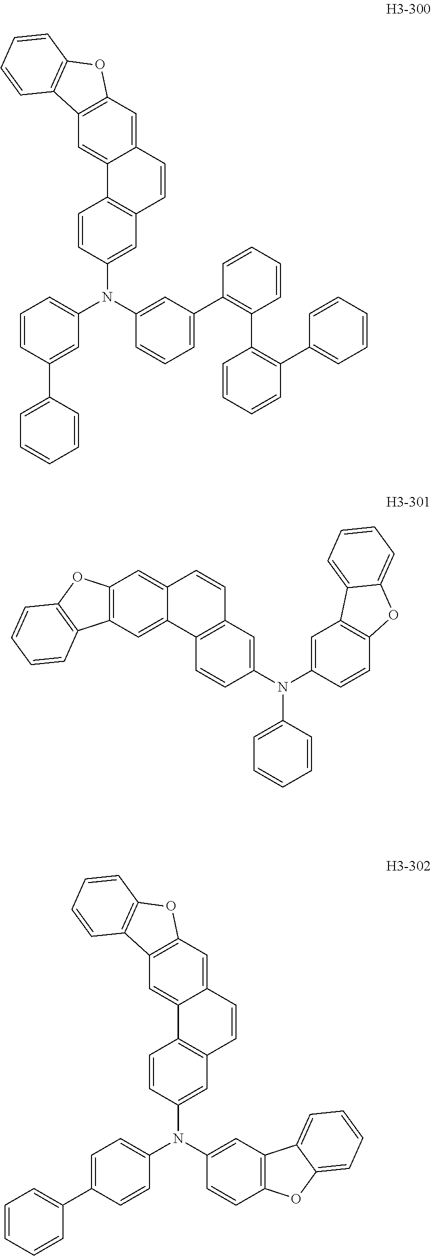 [SMILES: c1ccc(-c2cccc(N(c3cccc(-c4ccccc4-c4ccccc4-c4ccccc4)c3)c3ccc4c(ccc5cc6oc7ccccc7c6cc54)c3)c2)cc1 ; c1ccc(N(c2ccc3c(ccc4cc5oc6ccccc6c5cc43)c2)c2ccc3oc4ccccc4c3c2)cc1 ; c1ccc(-c2ccc(N(c3ccc4c(ccc5cc6oc7ccccc7c6cc54)c3)c3ccc4oc5ccccc5c4c3)cc2)cc1]

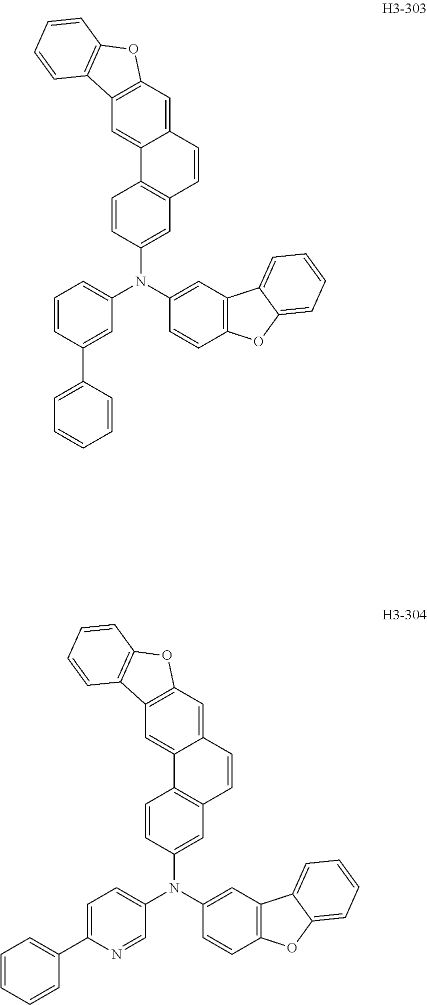 [SMILES: c1ccc(-c2cccc(N(c3ccc4c(ccc5cc6oc7ccccc7c6cc54)c3)c3ccc4oc5ccccc5c4c3)c2)cc1 ; c1ccc(-c2ccc(N(c3ccc4c(ccc5cc6oc7ccccc7c6cc54)c3)c3ccc4oc5ccccc5c4c3)cn2)cc1]

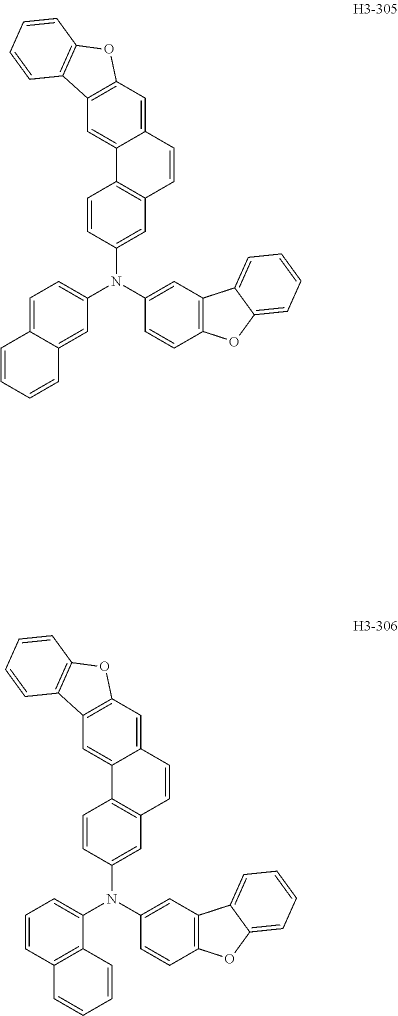 [SMILES: c1ccc2cc(N(c3ccc4c(ccc5cc6oc7ccccc7c6cc54)c3)c3ccc4oc5ccccc5c4c3)ccc2c1 ; c1ccc2c(N(c3ccc4c(ccc5cc6oc7ccccc7c6cc54)c3)c3ccc4oc5ccccc5c4c3)cccc2c1]

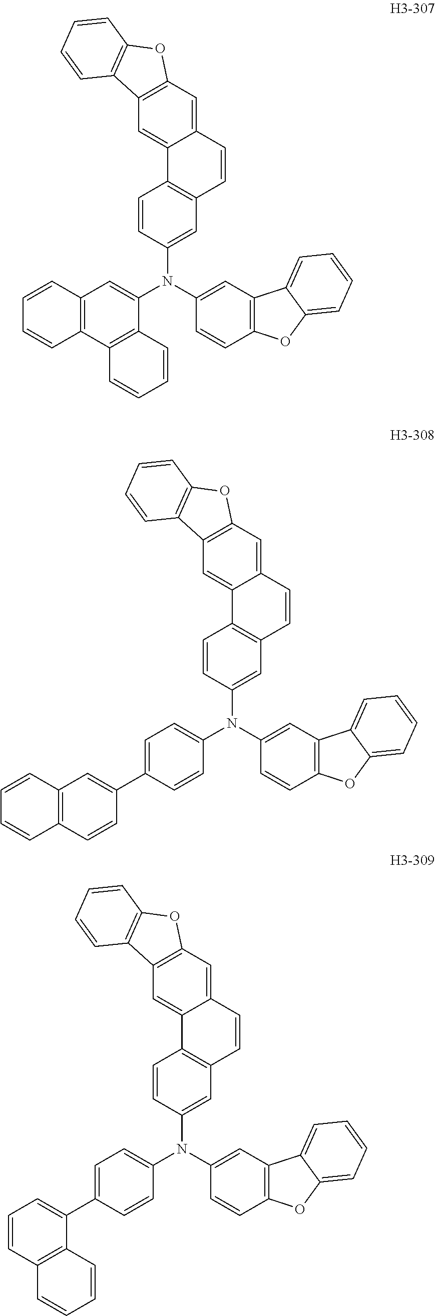 [SMILES: c1ccc2c(c1)cc(N(c1ccc3c(ccc4cc5oc6ccccc6c5cc43)c1)c1ccc3oc4ccccc4c3c1)c1ccccc12 ; c1ccc2cc(-c3ccc(N(c4ccc5c(ccc6cc7oc8ccccc8c7cc65)c4)c4ccc5oc6ccccc6c5c4)cc3)ccc2c1 ; c1ccc2c(-c3ccc(N(c4ccc5c(ccc6cc7oc8ccccc8c7cc65)c4)c4ccc5oc6ccccc6c5c4)cc3)cccc2c1]

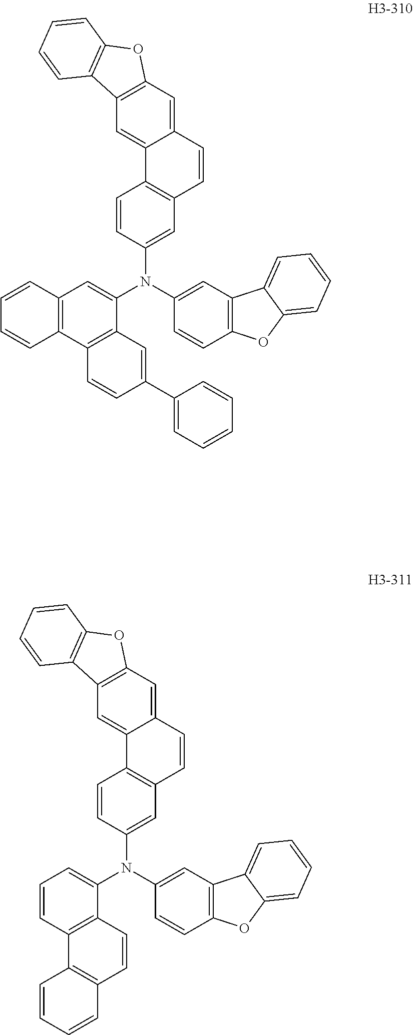 [SMILES: c1ccc(-c2ccc3c(c2)c(N(c2ccc4c(ccc5cc6oc7ccccc7c6cc54)c2)c2ccc4oc5ccccc5c4c2)cc2ccccc23)cc1 ; c1ccc2c(c1)ccc1c(N(c3ccc4c(ccc5cc6oc7ccccc7c6cc54)c3)c3ccc4oc5ccccc5c4c3)cccc12]

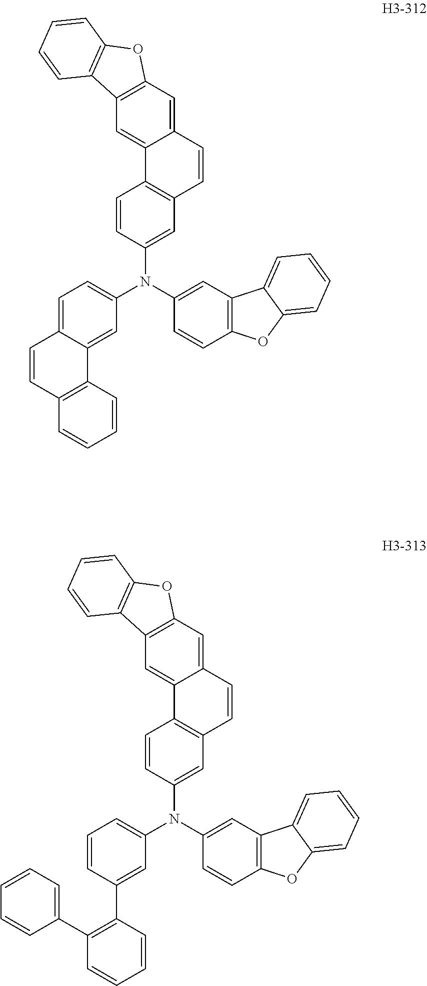 [SMILES: c1ccc2c(c1)ccc1ccc(N(c3ccc4c(ccc5cc6oc7ccccc7c6cc54)c3)c3ccc4oc5ccccc5c4c3)cc12 ; c1ccc(-c2ccccc2-c2cccc(N(c3ccc4c(ccc5cc6oc7ccccc7c6cc54)c3)c3ccc4oc5ccccc5c4c3)c2)cc1]

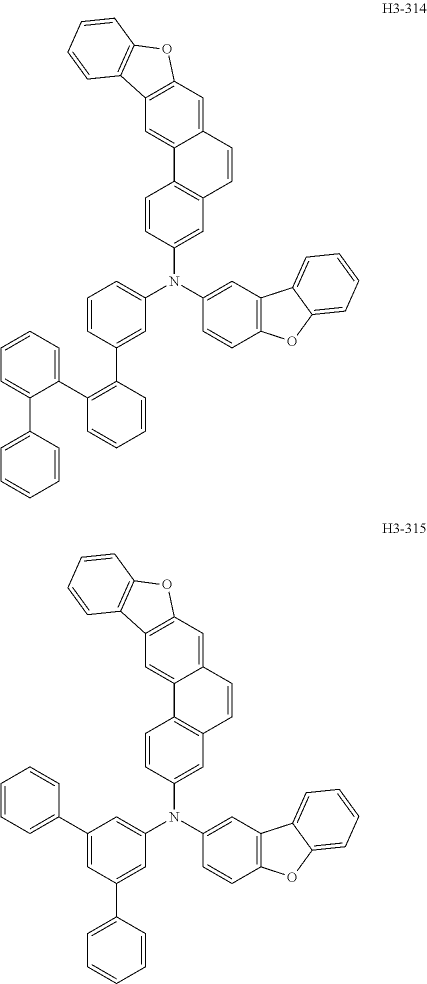 [SMILES: c1ccc(-c2ccccc2-c2ccccc2-c2cccc(N(c3ccc4c(ccc5cc6oc7ccccc7c6cc54)c3)c3ccc4oc5ccccc5c4c3)c2)cc1 ; c1ccc(-c2cc(-c3ccccc3)cc(N(c3ccc4c(ccc5cc6oc7ccccc7c6cc54)c3)c3ccc4oc5ccccc5c4c3)c2)cc1]

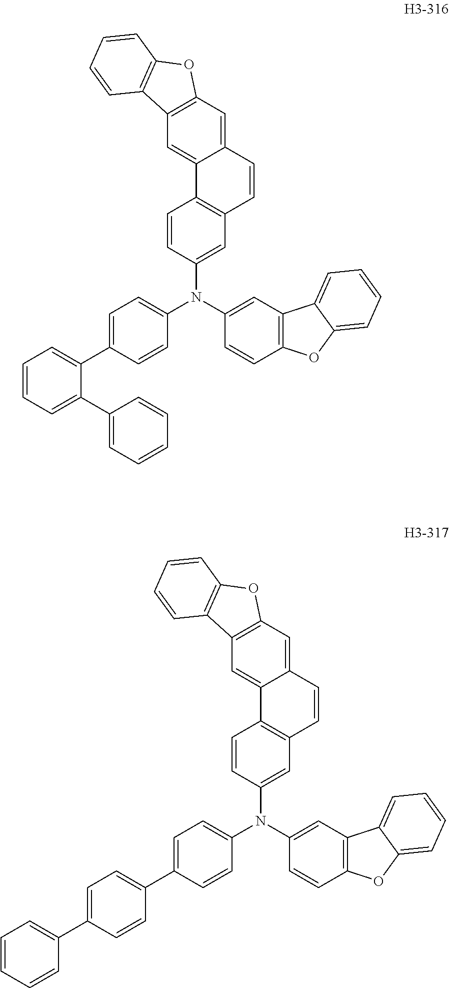 [SMILES: c1ccc(-c2ccccc2-c2ccc(N(c3ccc4c(ccc5cc6oc7ccccc7c6cc54)c3)c3ccc4oc5ccccc5c4c3)cc2)cc1 ; c1ccc(-c2ccc(-c3ccc(N(c4ccc5c(ccc6cc7oc8ccccc8c7cc65)c4)c4ccc5oc6ccccc6c5c4)cc3)cc2)cc1]

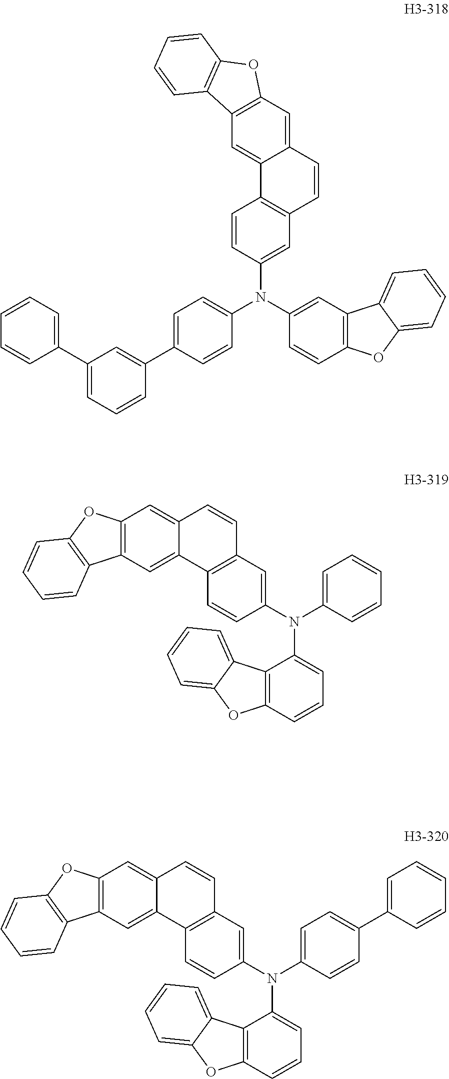 [SMILES: c1ccc(-c2cccc(-c3ccc(N(c4ccc5c(ccc6cc7oc8ccccc8c7cc65)c4)c4ccc5oc6ccccc6c5c4)cc3)c2)cc1 ; c1ccc(N(c2ccc3c(ccc4cc5oc6ccccc6c5cc43)c2)c2cccc3oc4ccccc4c23)cc1 ; c1ccc(-c2ccc(N(c3ccc4c(ccc5cc6oc7ccccc7c6cc54)c3)c3cccc4oc5ccccc5c34)cc2)cc1]

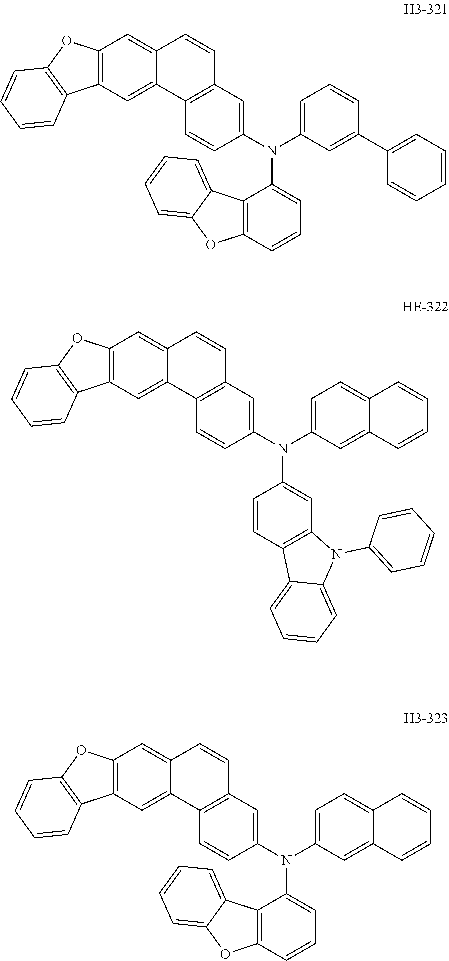 [SMILES: c1ccc(-c2cccc(N(c3ccc4c(ccc5cc6oc7ccccc7c6cc54)c3)c3cccc4oc5ccccc5c34)c2)cc1 ; c1ccc(-n2c3ccccc3c3ccc(N(c4ccc5ccccc5c4)c4ccc5c(ccc6cc7oc8ccccc8c7cc65)c4)cc32)cc1 ; c1ccc2cc(N(c3ccc4c(ccc5cc6oc7ccccc7c6cc54)c3)c3cccc4oc5ccccc5c34)ccc2c1]

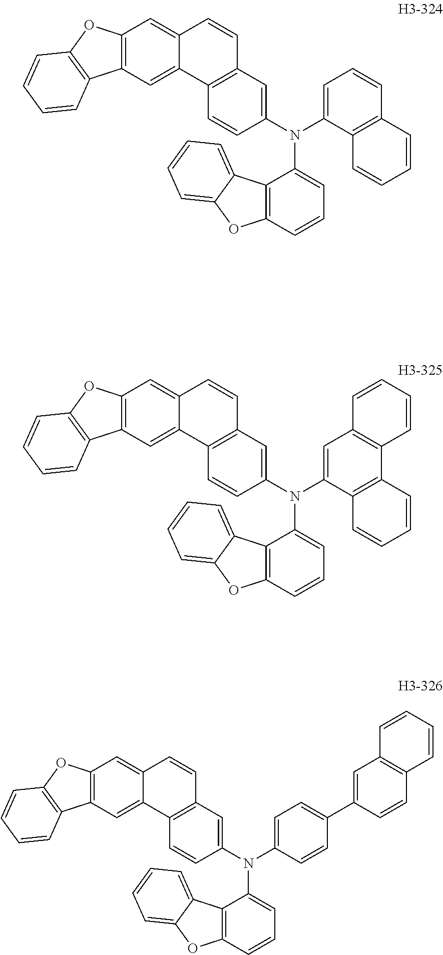 [SMILES: c1ccc2c(N(c3ccc4c(ccc5cc6oc7ccccc7c6cc54)c3)c3cccc4oc5ccccc5c34)cccc2c1 ; c1ccc2c(c1)cc(N(c1ccc3c(ccc4cc5oc6ccccc6c5cc43)c1)c1cccc3oc4ccccc4c13)c1ccccc12 ; c1ccc2cc(-c3ccc(N(c4ccc5c(ccc6cc7oc8ccccc8c7cc65)c4)c4cccc5oc6ccccc6c45)cc3)ccc2c1]

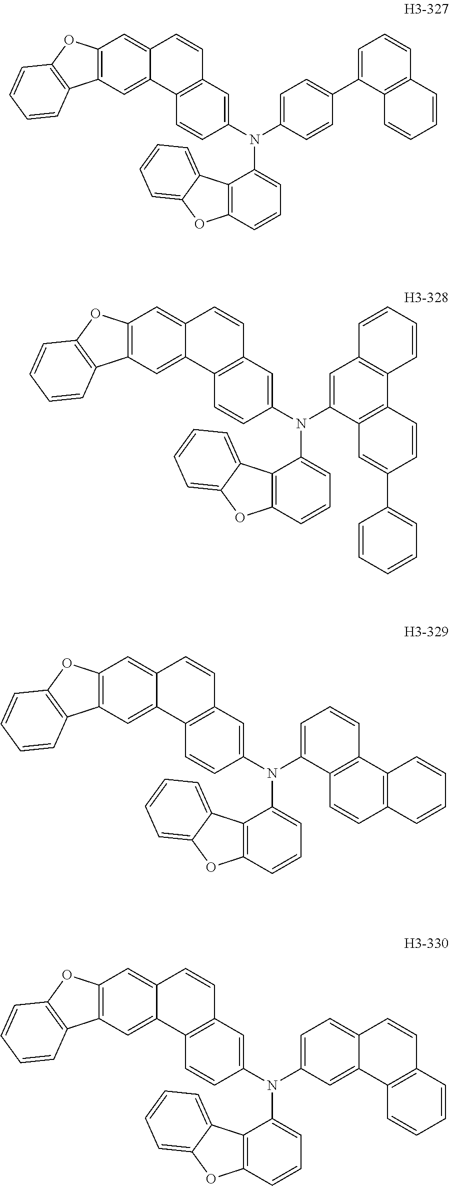 [SMILES: c1ccc2c(-c3ccc(N(c4ccc5c(ccc6cc7oc8ccccc8c7cc65)c4)c4cccc5oc6ccccc6c45)cc3)cccc2c1 ; c1ccc(-c2ccc3c(c2)c(N(c2ccc4c(ccc5cc6oc7ccccc7c6cc54)c2)c2cccc4oc5ccccc5c24)cc2ccccc23)cc1 ; c1ccc2c(c1)ccc1c(N(c3ccc4c(ccc5cc6oc7ccccc7c6cc54)c3)c3cccc4oc5ccccc5c34)cccc12 ; c1ccc2c(c1)ccc1ccc(N(c3ccc4c(ccc5cc6oc7ccccc7c6cc54)c3)c3cccc4oc5ccccc5c34)cc12]

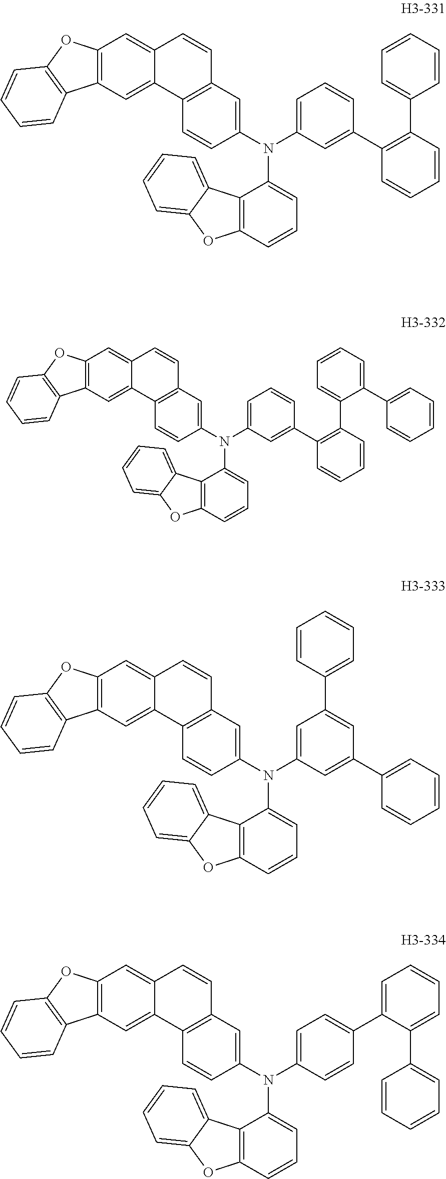 [SMILES: c1ccc(-c2ccccc2-c2cccc(N(c3ccc4c(ccc5cc6oc7ccccc7c6cc54)c3)c3cccc4oc5ccccc5c34)c2)cc1 ; c1ccc(-c2ccccc2-c2ccccc2-c2cccc(N(c3ccc4c(ccc5cc6oc7ccccc7c6cc54)c3)c3cccc4oc5ccccc5c34)c2)cc1 ; c1ccc(-c2cc(-c3ccccc3)cc(N(c3ccc4c(ccc5cc6oc7ccccc7c6cc54)c3)c3cccc4oc5ccccc5c34)c2)cc1 ; c1ccc(-c2ccccc2-c2ccc(N(c3ccc4c(ccc5cc6oc7ccccc7c6cc54)c3)c3cccc4oc5ccccc5c34)cc2)cc1]

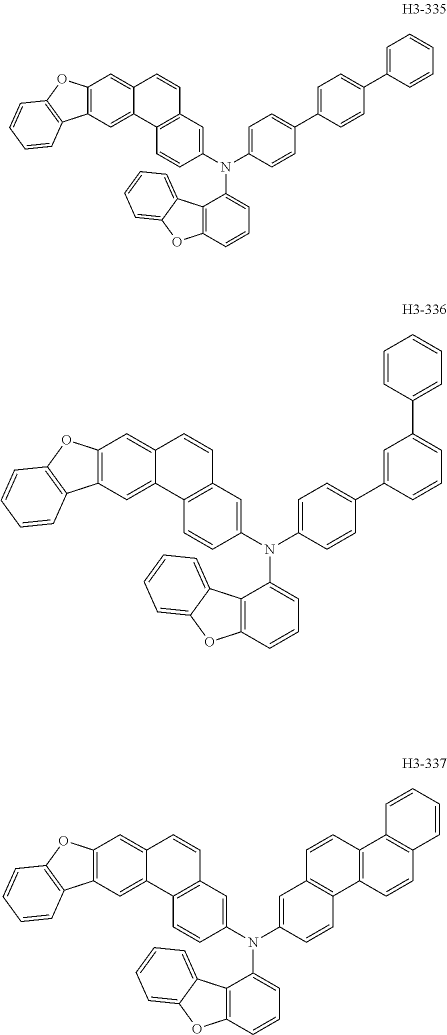 [SMILES: c1ccc(-c2ccc(-c3ccc(N(c4ccc5c(ccc6cc7oc8ccccc8c7cc65)c4)c4cccc5oc6ccccc6c45)cc3)cc2)cc1 ; c1ccc(-c2cccc(-c3ccc(N(c4ccc5c(ccc6cc7oc8ccccc8c7cc65)c4)c4cccc5oc6ccccc6c45)cc3)c2)cc1 ; c1ccc2c(c1)ccc1c3ccc(N(c4ccc5c(ccc6cc7oc8ccccc8c7cc65)c4)c4cccc5oc6ccccc6c45)cc3ccc21]

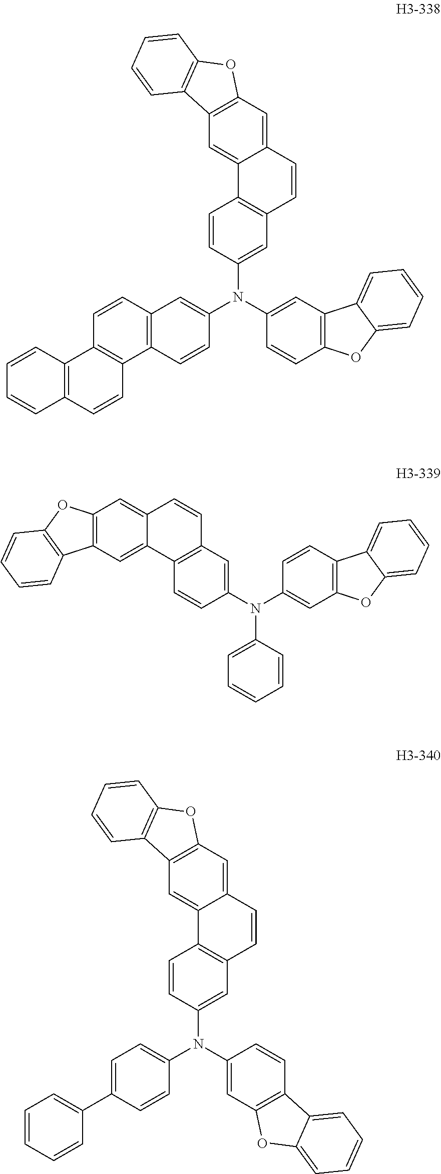 [SMILES: c1ccc2c(c1)ccc1c3ccc(N(c4ccc5c(ccc6cc7oc8ccccc8c7cc65)c4)c4ccc5oc6ccccc6c5c4)cc3ccc21 ; c1ccc(N(c2ccc3c(ccc4cc5oc6ccccc6c5cc43)c2)c2ccc3c(c2)oc2ccccc23)cc1 ; c1ccc(-c2ccc(N(c3ccc4c(ccc5cc6oc7ccccc7c6cc54)c3)c3ccc4c(c3)oc3ccccc34)cc2)cc1]

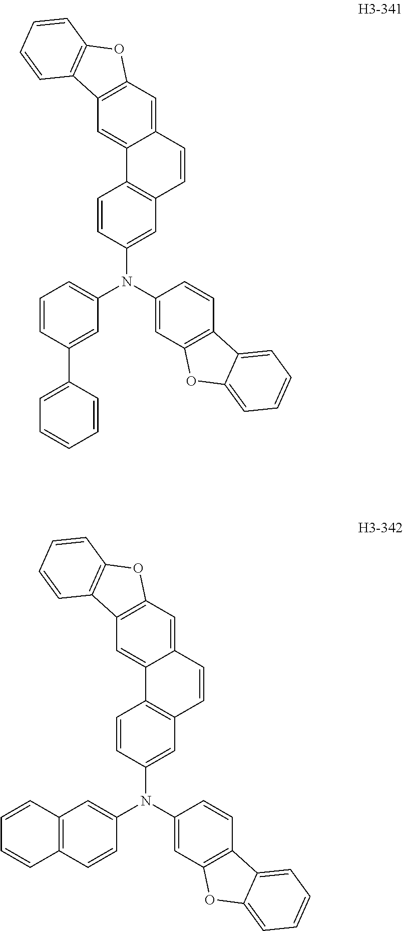 [SMILES: c1ccc(-c2cccc(N(c3ccc4c(ccc5cc6oc7ccccc7c6cc54)c3)c3ccc4c(c3)oc3ccccc34)c2)cc1 ; c1ccc2cc(N(c3ccc4c(ccc5cc6oc7ccccc7c6cc54)c3)c3ccc4c(c3)oc3ccccc34)ccc2c1]

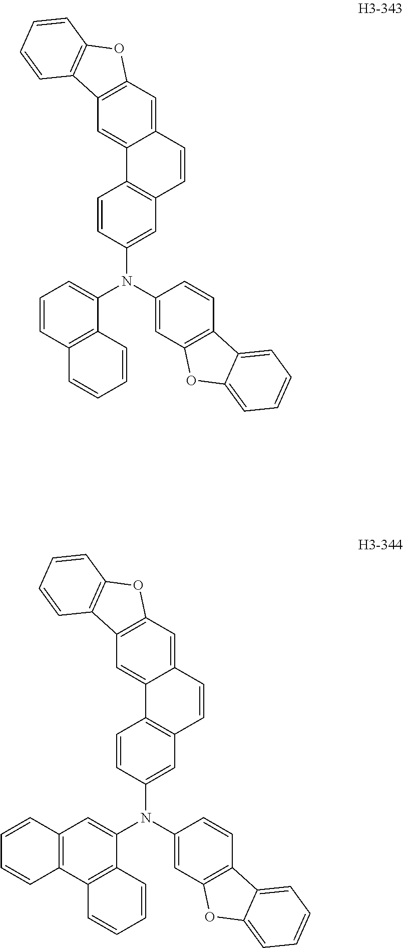 [SMILES: c1ccc2c(N(c3ccc4c(ccc5cc6oc7ccccc7c6cc54)c3)c3ccc4c(c3)oc3ccccc34)cccc2c1 ; c1ccc2c(c1)cc(N(c1ccc3c(ccc4cc5oc6ccccc6c5cc43)c1)c1ccc3c(c1)oc1ccccc13)c1ccccc12]

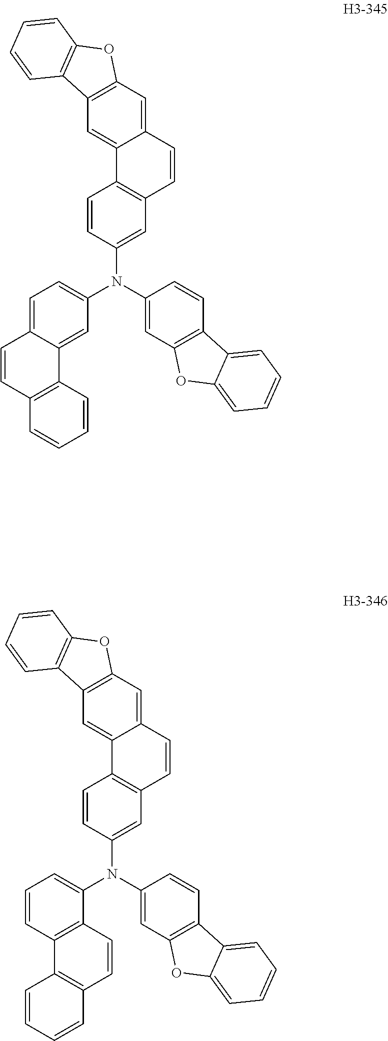 [SMILES: c1ccc2c(c1)ccc1ccc(N(c3ccc4c(ccc5cc6oc7ccccc7c6cc54)c3)c3ccc4c(c3)oc3ccccc34)cc12 ; c1ccc2c(c1)ccc1c(N(c3ccc4c(ccc5cc6oc7ccccc7c6cc54)c3)c3ccc4c(c3)oc3ccccc34)cccc12]

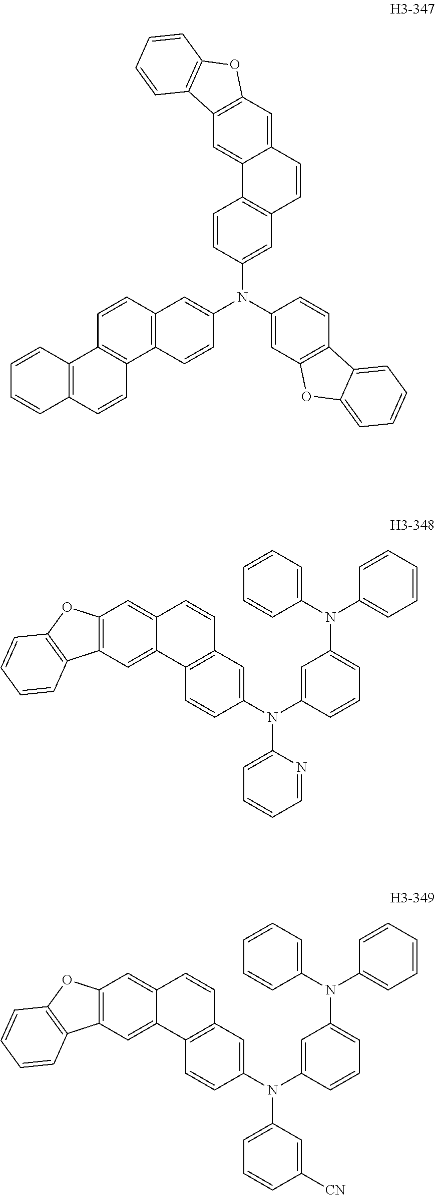 [SMILES: c1ccc2c(c1)ccc1c3ccc(N(c4ccc5c(ccc6cc7oc8ccccc8c7cc65)c4)c4ccc5c(c4)oc4ccccc45)cc3ccc21 ; c1ccc(N(c2ccccc2)c2cccc(N(c3ccc4c(ccc5cc6oc7ccccc7c6cc54)c3)c3ccccn3)c2)cc1 ; N#Cc1cccc(N(c2cccc(N(c3ccccc3)c3ccccc3)c2)c2ccc3c(ccc4cc5oc6ccccc6c5cc43)c2)c1]

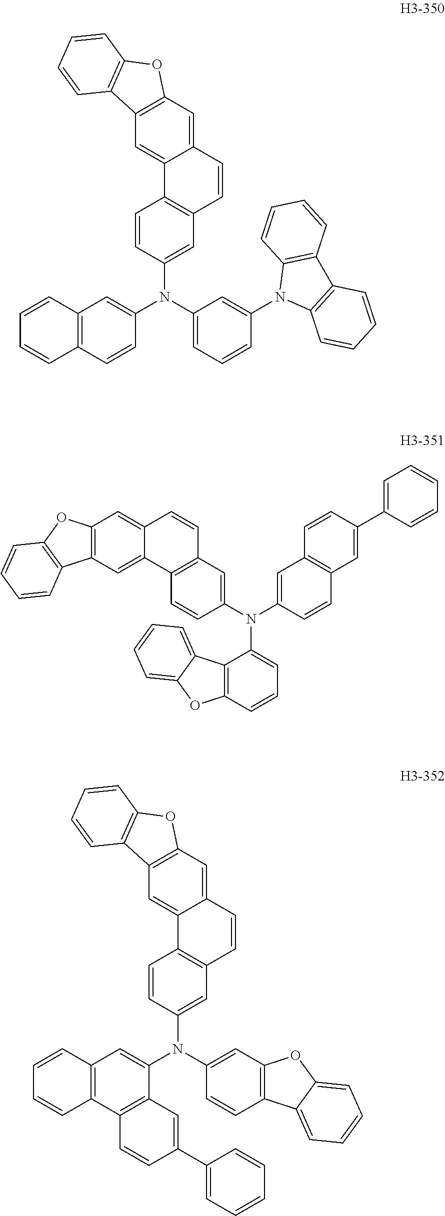 [SMILES: c1cc(N(c2ccc3ccccc3c2)c2ccc3c(ccc4cc5oc6ccccc6c5cc43)c2)cc(-n2c3ccccc3c3ccccc32)c1 ; c1ccc(-c2ccc3cc(N(c4ccc5c(ccc6cc7oc8ccccc8c7cc65)c4)c4cccc5oc6ccccc6c45)ccc3c2)cc1 ; c1ccc(-c2ccc3c(c2)c(N(c2ccc4c(ccc5cc6oc7ccccc7c6cc54)c2)c2ccc4c(c2)oc2ccccc24)cc2ccccc23)cc1]

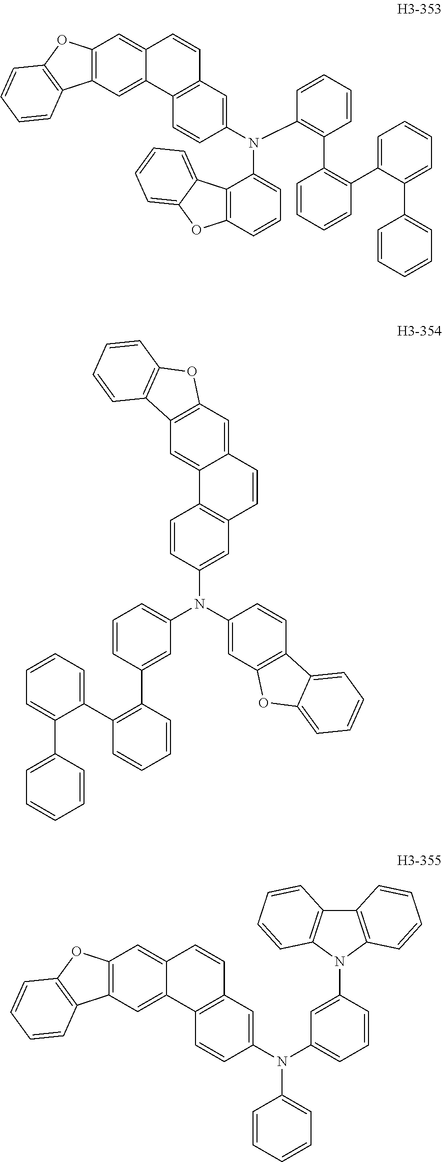 [SMILES: c1ccc(-c2ccccc2-c2ccccc2-c2ccccc2N(c2ccc3c(ccc4cc5oc6ccccc6c5cc43)c2)c2cccc3oc4ccccc4c23)cc1 ; c1ccc(-c2ccccc2-c2ccccc2-c2cccc(N(c3ccc4c(ccc5cc6oc7ccccc7c6cc54)c3)c3ccc4c(c3)oc3ccccc34)c2)cc1 ; c1ccc(N(c2cccc(-n3c4ccccc4c4ccccc43)c2)c2ccc3c(ccc4cc5oc6ccccc6c5cc43)c2)cc1]

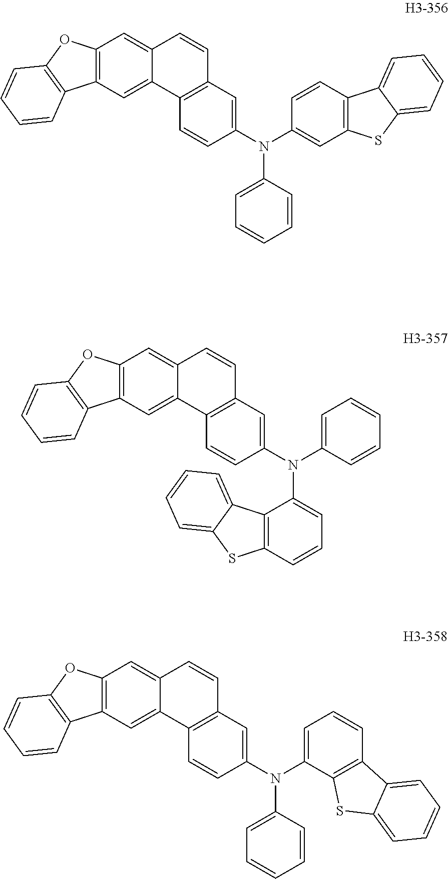 [SMILES: c1ccc(N(c2ccc3c(ccc4cc5oc6ccccc6c5cc43)c2)c2ccc3c(c2)sc2ccccc23)cc1 ; c1ccc(N(c2ccc3c(ccc4cc5oc6ccccc6c5cc43)c2)c2cccc3sc4ccccc4c23)cc1 ; c1ccc(N(c2ccc3c(ccc4cc5oc6ccccc6c5cc43)c2)c2cccc3c2sc2ccccc23)cc1]

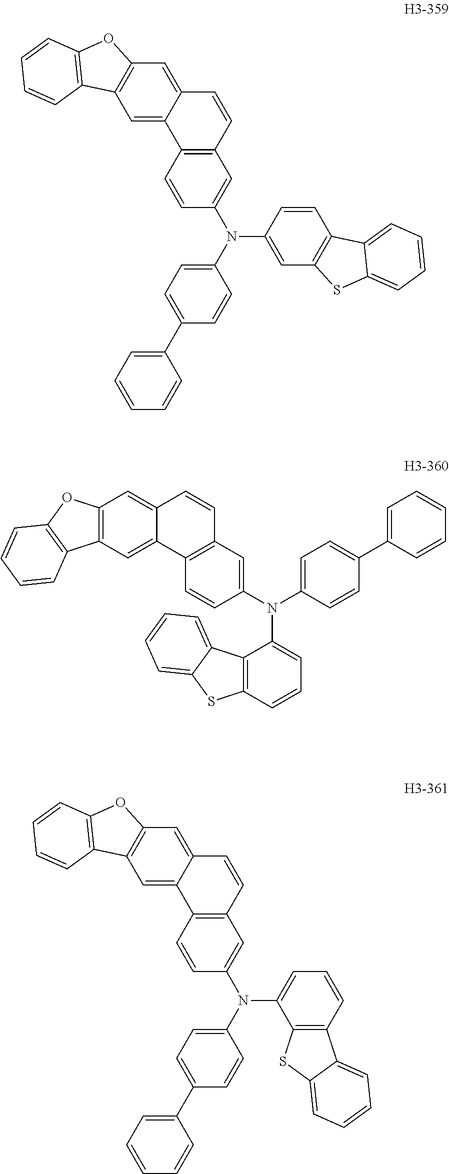 [SMILES: c1ccc(-c2ccc(N(c3ccc4c(ccc5cc6oc7ccccc7c6cc54)c3)c3ccc4c(c3)sc3ccccc34)cc2)cc1 ; c1ccc(-c2ccc(N(c3ccc4c(ccc5cc6oc7ccccc7c6cc54)c3)c3cccc4sc5ccccc5c34)cc2)cc1 ; c1ccc(-c2ccc(N(c3ccc4c(ccc5cc6oc7ccccc7c6cc54)c3)c3cccc4c3sc3ccccc34)cc2)cc1]

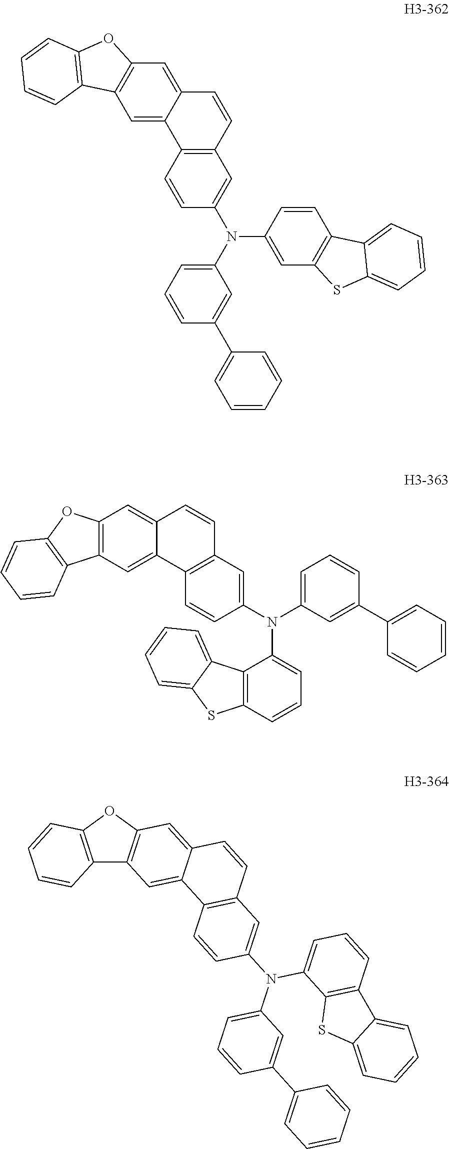 [SMILES: c1ccc(-c2cccc(N(c3ccc4c(ccc5cc6oc7ccccc7c6cc54)c3)c3ccc4c(c3)sc3ccccc34)c2)cc1 ; c1ccc(-c2cccc(N(c3ccc4c(ccc5cc6oc7ccccc7c6cc54)c3)c3cccc4sc5ccccc5c34)c2)cc1 ; c1ccc(-c2cccc(N(c3ccc4c(ccc5cc6oc7ccccc7c6cc54)c3)c3cccc4c3sc3ccccc34)c2)cc1]

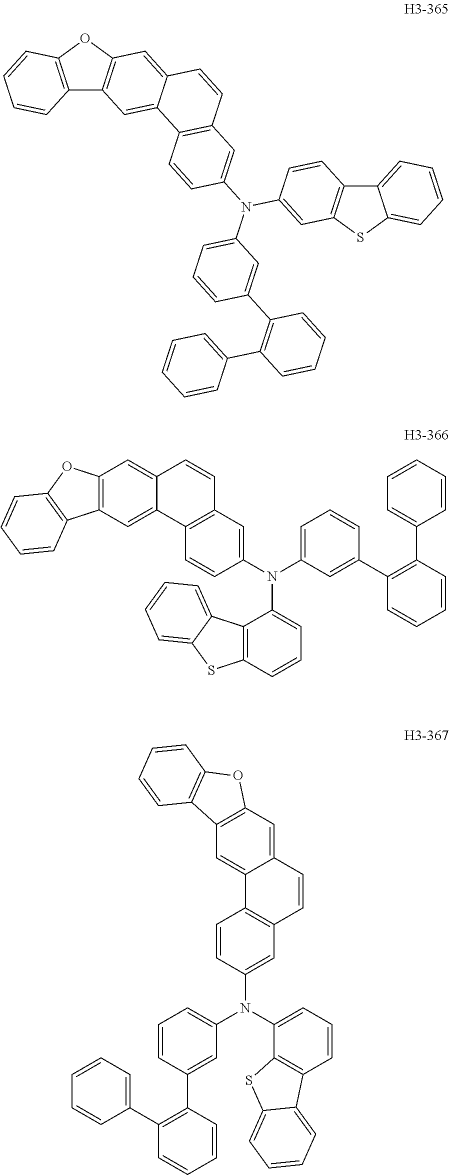 [SMILES: c1ccc(-c2ccccc2-c2cccc(N(c3ccc4c(ccc5cc6oc7ccccc7c6cc54)c3)c3ccc4c(c3)sc3ccccc34)c2)cc1 ; c1ccc(-c2ccccc2-c2cccc(N(c3ccc4c(ccc5cc6oc7ccccc7c6cc54)c3)c3cccc4sc5ccccc5c34)c2)cc1 ; c1ccc(-c2ccccc2-c2cccc(N(c3ccc4c(ccc5cc6oc7ccccc7c6cc54)c3)c3cccc4c3sc3ccccc34)c2)cc1]

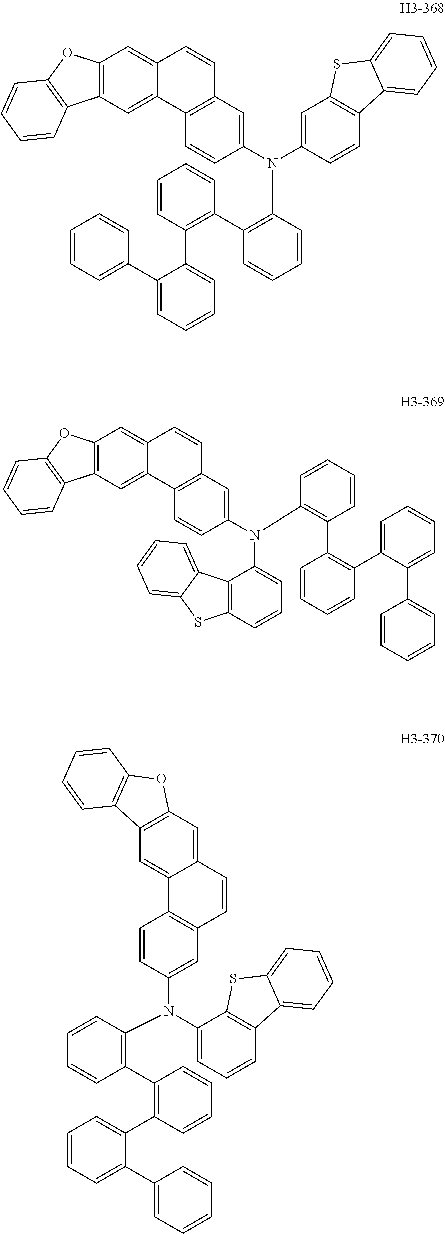 [SMILES: c1ccc(-c2ccccc2-c2ccccc2-c2ccccc2N(c2ccc3c(ccc4cc5oc6ccccc6c5cc43)c2)c2ccc3c(c2)sc2ccccc23)cc1 ; c1ccc(-c2ccccc2-c2ccccc2-c2ccccc2N(c2ccc3c(ccc4cc5oc6ccccc6c5cc43)c2)c2cccc3sc4ccccc4c23)cc1 ; c1ccc(-c2ccccc2-c2ccccc2-c2ccccc2N(c2ccc3c(ccc4cc5oc6ccccc6c5cc43)c2)c2cccc3c2sc2ccccc23)cc1]

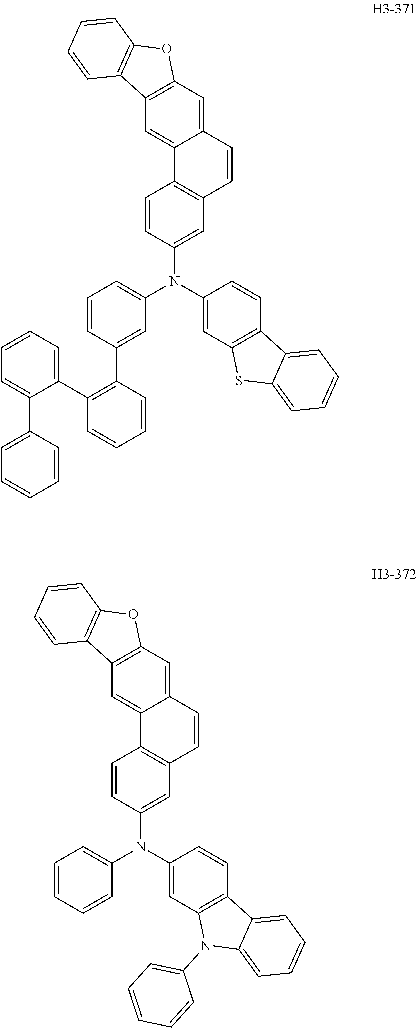 [SMILES: c1ccc(-c2ccccc2-c2ccccc2-c2cccc(N(c3ccc4c(ccc5cc6oc7ccccc7c6cc54)c3)c3ccc4c(c3)sc3ccccc34)c2)cc1 ; c1ccc(N(c2ccc3c(ccc4cc5oc6ccccc6c5cc43)c2)c2ccc3c4ccccc4n(-c4ccccc4)c3c2)cc1]

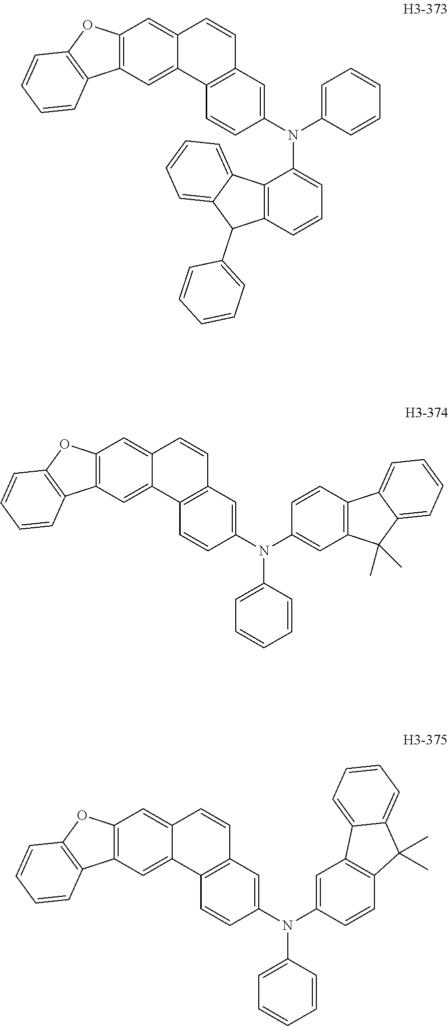 [SMILES: c1ccc(C2c3ccccc3-c3c2cccc3N(c2ccccc2)c2ccc3c(ccc4cc5oc6ccccc6c5cc43)c2)cc1 ; CC1(C)c2ccccc2-c2ccc(N(c3ccccc3)c3ccc4c(ccc5cc6oc7ccccc7c6cc54)c3)cc21 ; CC1(C)c2ccccc2-c2cc(N(c3ccccc3)c3ccc4c(ccc5cc6oc7ccccc7c6cc54)c3)ccc21]

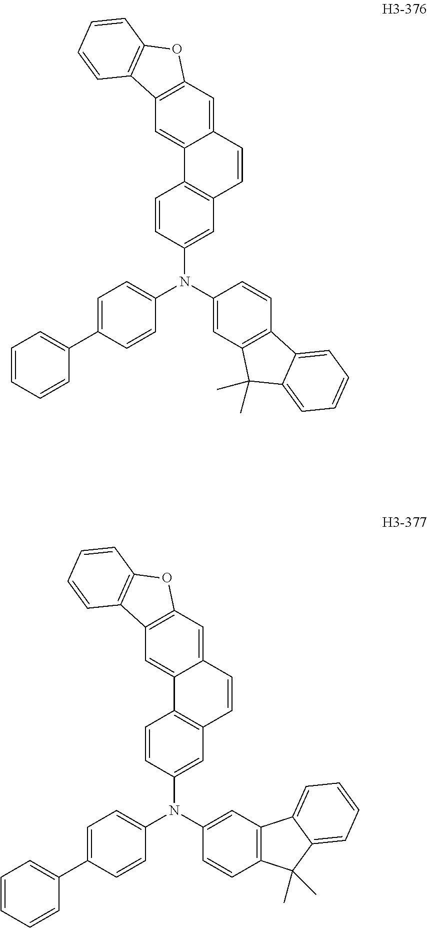 [SMILES: CC1(C)c2ccccc2-c2ccc(N(c3ccc(-c4ccccc4)cc3)c3ccc4c(ccc5cc6oc7ccccc7c6cc54)c3)cc21 ; CC1(C)c2ccccc2-c2cc(N(c3ccc(-c4ccccc4)cc3)c3ccc4c(ccc5cc6oc7ccccc7c6cc54)c3)ccc21]

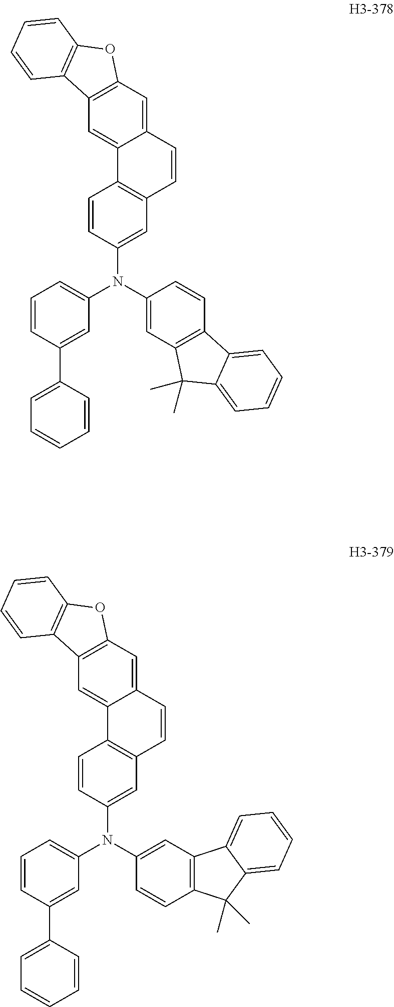 [SMILES: CC1(C)c2ccccc2-c2ccc(N(c3cccc(-c4ccccc4)c3)c3ccc4c(ccc5cc6oc7ccccc7c6cc54)c3)cc21 ; CC1(C)c2ccccc2-c2cc(N(c3cccc(-c4ccccc4)c3)c3ccc4c(ccc5cc6oc7ccccc7c6cc54)c3)ccc21]

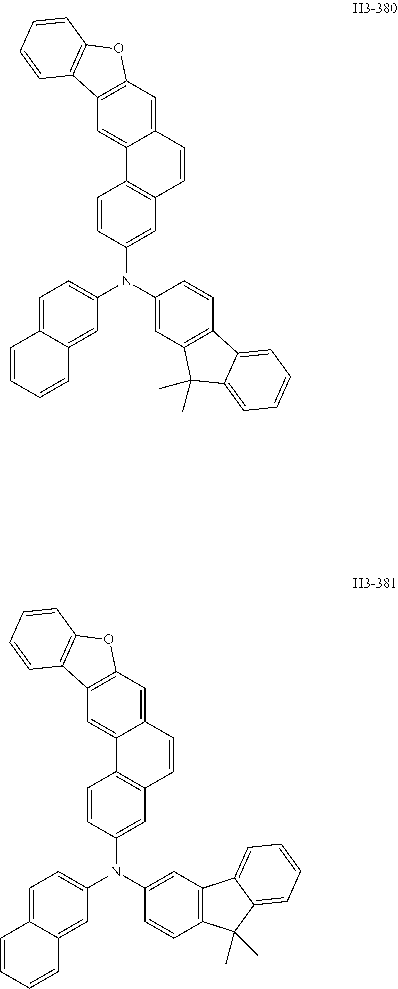 [SMILES: CC1(C)c2ccccc2-c2ccc(N(c3ccc4ccccc4c3)c3ccc4c(ccc5cc6oc7ccccc7c6cc54)c3)cc21 ; CC1(C)c2ccccc2-c2cc(N(c3ccc4ccccc4c3)c3ccc4c(ccc5cc6oc7ccccc7c6cc54)c3)ccc21]

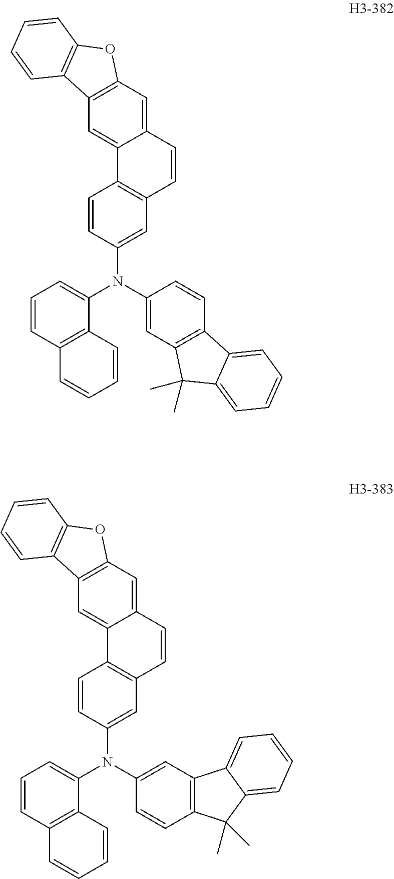 [SMILES: CC1(C)c2ccccc2-c2ccc(N(c3ccc4c(ccc5cc6oc7ccccc7c6cc54)c3)c3cccc4ccccc34)cc21 ; CC1(C)c2ccccc2-c2cc(N(c3ccc4c(ccc5cc6oc7ccccc7c6cc54)c3)c3cccc4ccccc34)ccc21]

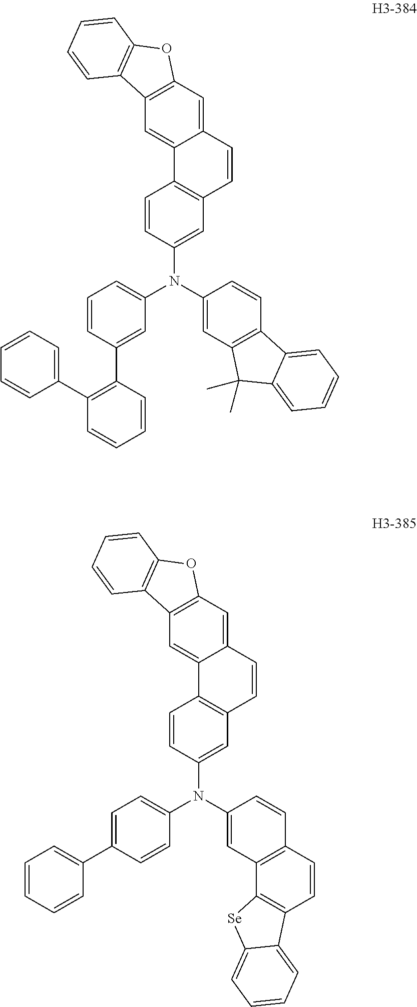 [SMILES: CC1(C)c2ccccc2-c2ccc(N(c3cccc(-c4ccccc4-c4ccccc4)c3)c3ccc4c(ccc5cc6oc7ccccc7c6cc54)c3)cc21 ; c1ccc(-c2ccc(N(c3ccc4c(ccc5cc6oc7ccccc7c6cc54)c3)c3ccc4ccc5c6ccccc6[se]c5c4c3)cc2)cc1]

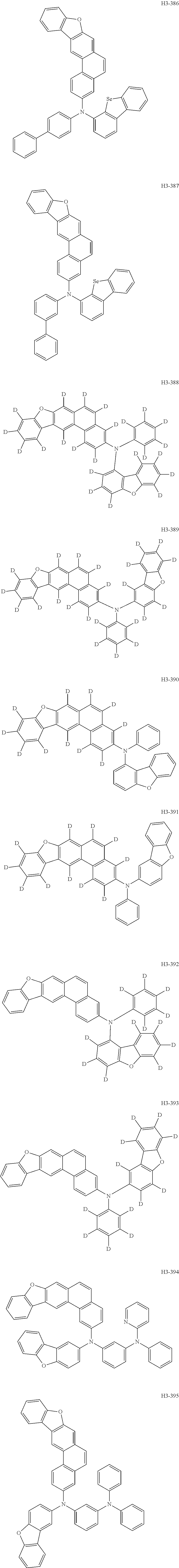

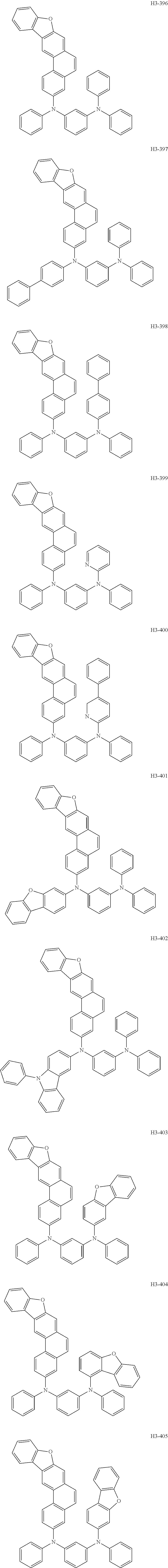

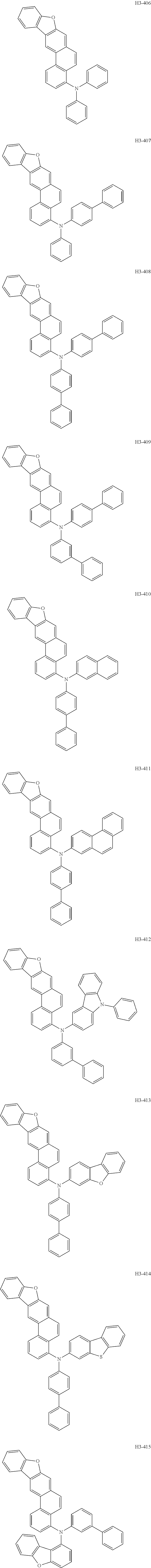

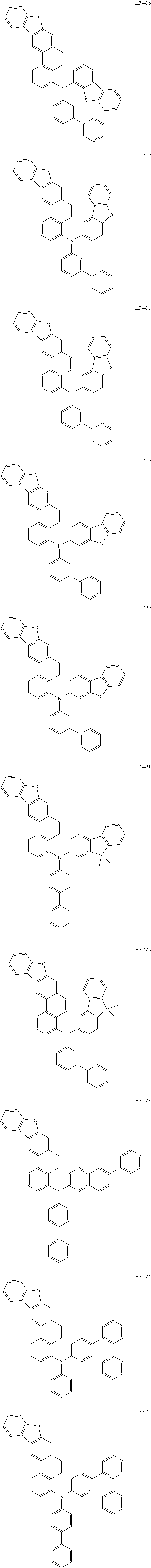

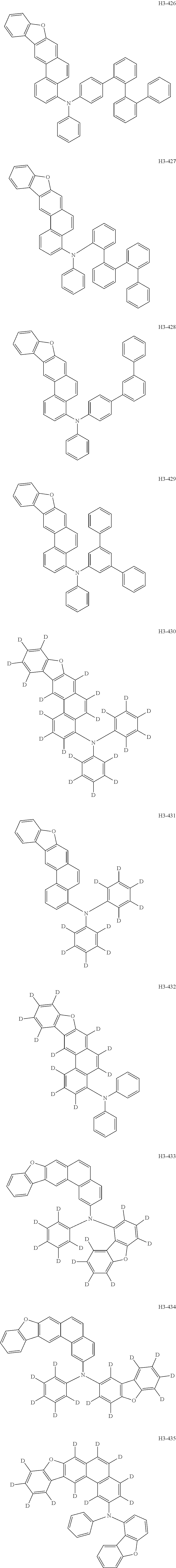

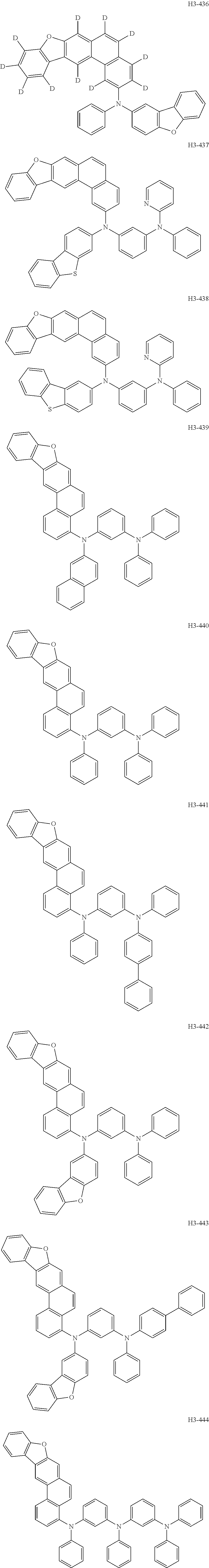

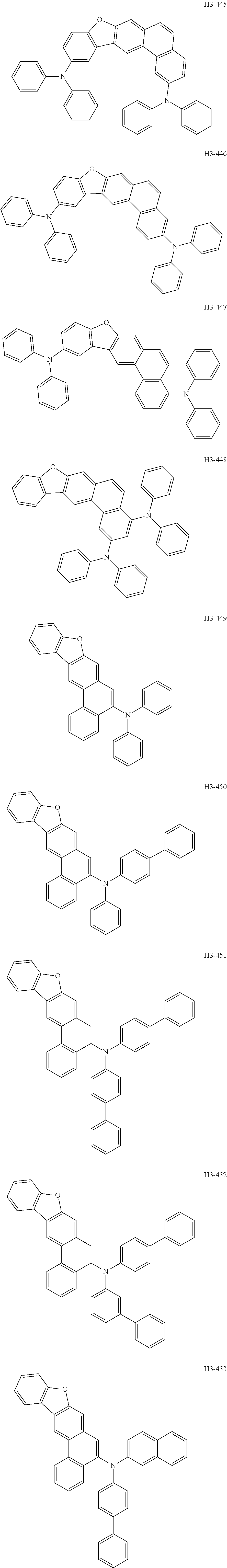

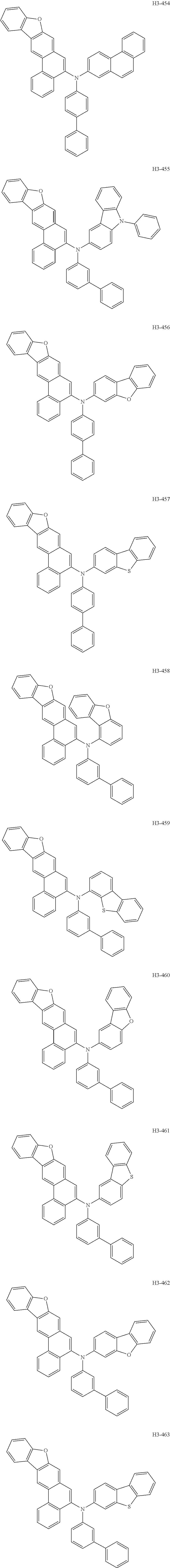

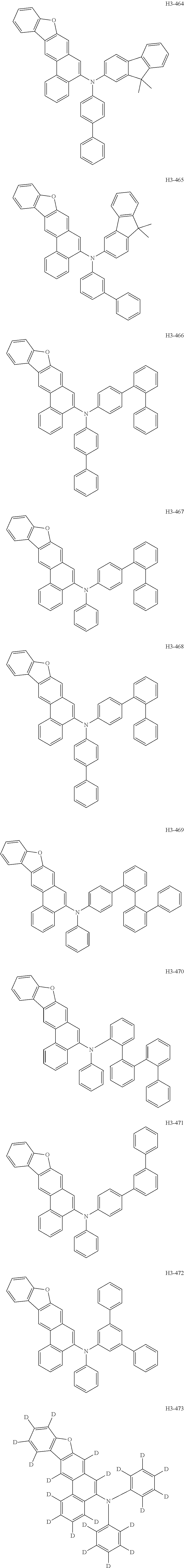

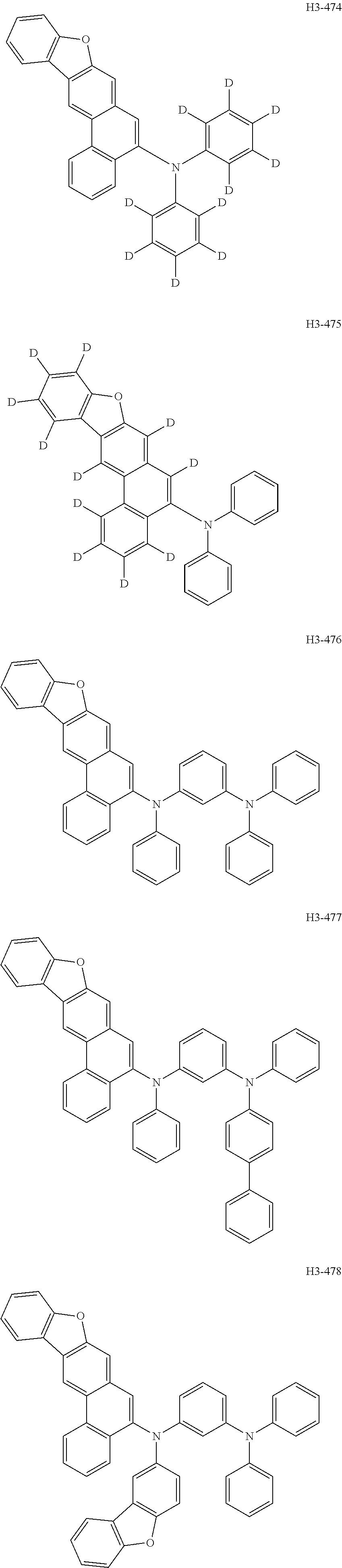 [SMILES: [2H]c1c([2H])c([2H])c(N(c2c([2H])c([2H])c([2H])c([2H])c2[2H])c2cc3cc4oc5ccccc5c4cc3c3ccccc23)c([2H])c1[2H] ; [2H]c1c([2H])c([2H])c2c(oc3c([2H])c4c([2H])c(N(c5ccccc5)c5ccccc5)c5c([2H])c([2H])c([2H])c([2H])c5c4c([2H])c32)c1[2H] ; c1ccc(N(c2ccccc2)c2cccc(N(c3ccccc3)c3cc4cc5oc6ccccc6c5cc4c4ccccc34)c2)cc1 ; c1ccc(-c2ccc(N(c3ccccc3)c3cccc(N(c4ccccc4)c4cc5cc6oc7ccccc7c6cc5c5ccccc45)c3)cc2)cc1 ; c1ccc(N(c2ccccc2)c2cccc(N(c3ccc4oc5ccccc5c4c3)c3cc4cc5oc6ccccc6c5cc4c4ccccc34)c2)cc1]

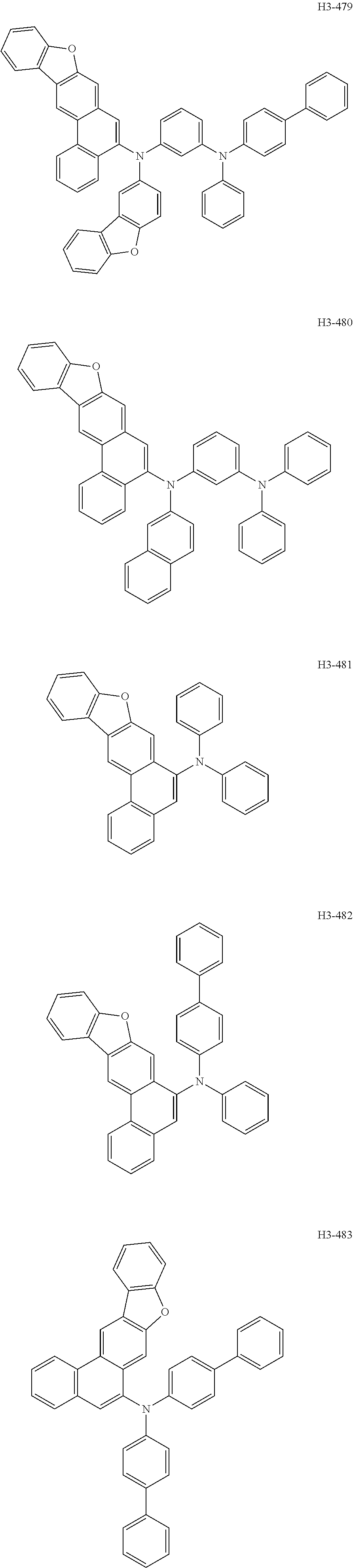 [SMILES: c1ccc(-c2ccc(N(c3ccccc3)c3cccc(N(c4ccc5oc6ccccc6c5c4)c4cc5cc6oc7ccccc7c6cc5c5ccccc45)c3)cc2)cc1 ; c1ccc(N(c2ccccc2)c2cccc(N(c3ccc4ccccc4c3)c3cc4cc5oc6ccccc6c5cc4c4ccccc34)c2)cc1 ; c1ccc(N(c2ccccc2)c2cc3ccccc3c3cc4c(cc23)oc2ccccc24)cc1 ; c1ccc(-c2ccc(N(c3ccccc3)c3cc4ccccc4c4cc5c(cc34)oc3ccccc35)cc2)cc1 ; c1ccc(-c2ccc(N(c3ccc(-c4ccccc4)cc3)c3cc4ccccc4c4cc5c(cc34)oc3ccccc35)cc2)cc1]

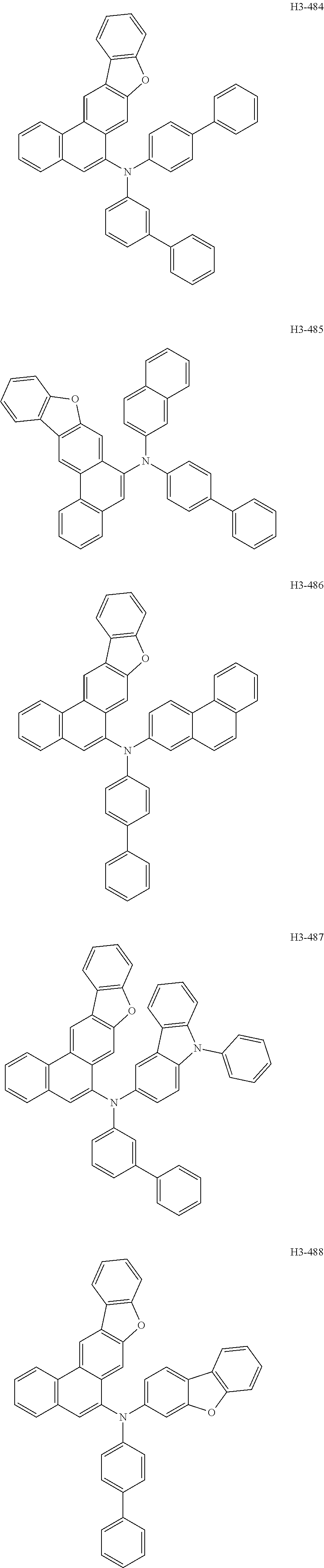 [SMILES: c1ccc(-c2ccc(N(c3cccc(-c4ccccc4)c3)c3cc4ccccc4c4cc5c(cc34)oc3ccccc35)cc2)cc1 ; c1ccc(-c2ccc(N(c3ccc4ccccc4c3)c3cc4ccccc4c4cc5c(cc34)oc3ccccc35)cc2)cc1 ; c1ccc(-c2ccc(N(c3ccc4c(ccc5ccccc54)c3)c3cc4ccccc4c4cc5c(cc34)oc3ccccc35)cc2)cc1 ; c1ccc(-c2cccc(N(c3ccc4c(c3)c3ccccc3n4-c3ccccc3)c3cc4ccccc4c4cc5c(cc34)oc3ccccc35)c2)cc1 ; c1ccc(-c2ccc(N(c3ccc4c(c3)oc3ccccc34)c3cc4ccccc4c4cc5c(cc34)oc3ccccc35)cc2)cc1]

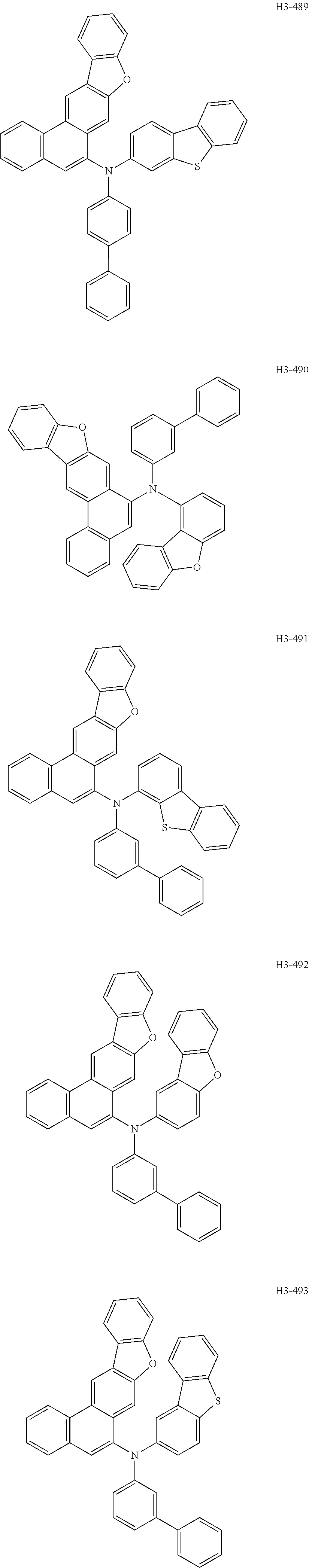 [SMILES: c1ccc(-c2ccc(N(c3ccc4c(c3)sc3ccccc34)c3cc4ccccc4c4cc5c(cc34)oc3ccccc35)cc2)cc1 ; c1ccc(-c2cccc(N(c3cc4ccccc4c4cc5c(cc34)oc3ccccc35)c3cccc4oc5ccccc5c34)c2)cc1 ; c1ccc(-c2cccc(N(c3cc4ccccc4c4cc5c(cc34)oc3ccccc35)c3cccc4c3sc3ccccc34)c2)cc1 ; c1ccc(-c2cccc(N(c3ccc4oc5ccccc5c4c3)c3cc4ccccc4c4cc5c(cc34)oc3ccccc35)c2)cc1 ; c1ccc(-c2cccc(N(c3ccc4sc5ccccc5c4c3)c3cc4ccccc4c4cc5c(cc34)oc3ccccc35)c2)cc1]

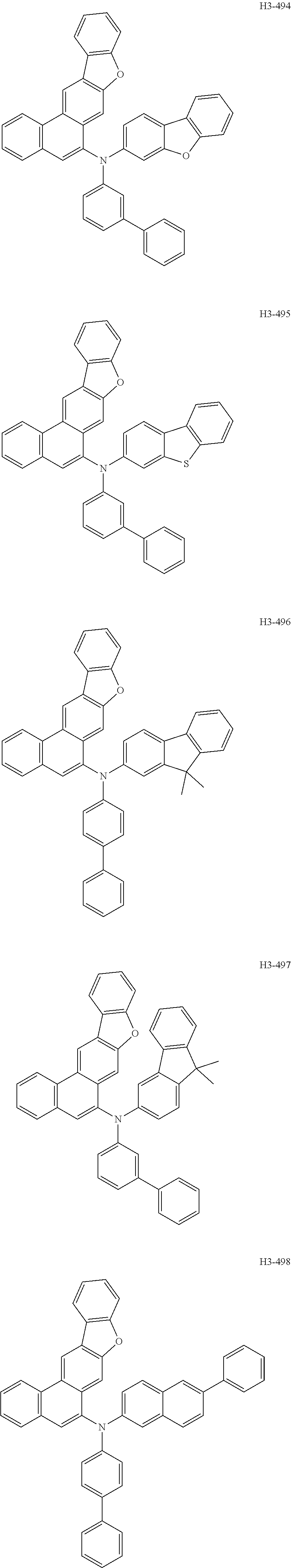 [SMILES: c1ccc(-c2cccc(N(c3ccc4c(c3)oc3ccccc34)c3cc4ccccc4c4cc5c(cc34)oc3ccccc35)c2)cc1 ; c1ccc(-c2cccc(N(c3ccc4c(c3)sc3ccccc34)c3cc4ccccc4c4cc5c(cc34)oc3ccccc35)c2)cc1 ; CC1(C)c2ccccc2-c2ccc(N(c3ccc(-c4ccccc4)cc3)c3cc4ccccc4c4cc5c(cc34)oc3ccccc35)cc21 ; CC1(C)c2ccccc2-c2cc(N(c3cccc(-c4ccccc4)c3)c3cc4ccccc4c4cc5c(cc34)oc3ccccc35)ccc21 ; c1ccc(-c2ccc(N(c3ccc4cc(-c5ccccc5)ccc4c3)c3cc4ccccc4c4cc5c(cc34)oc3ccccc35)cc2)cc1]

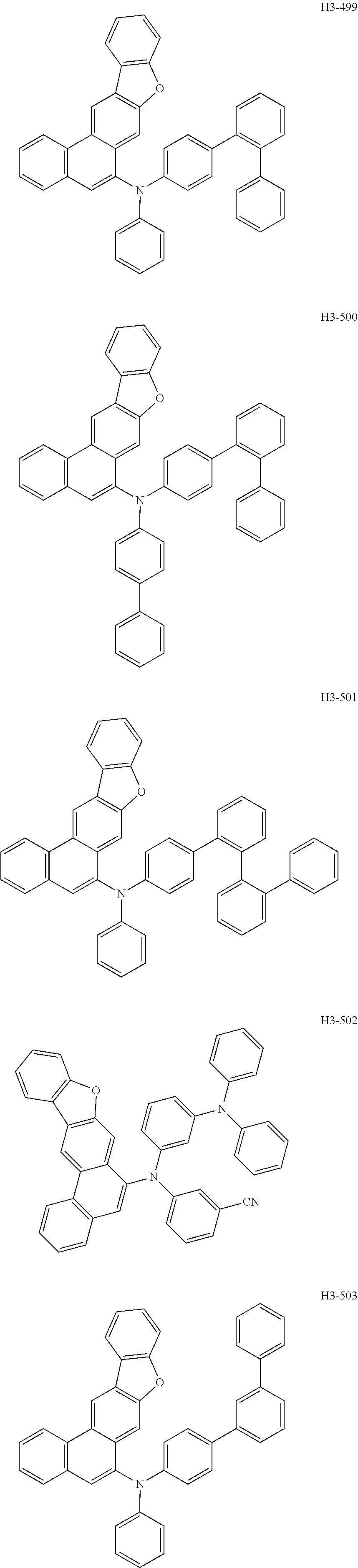

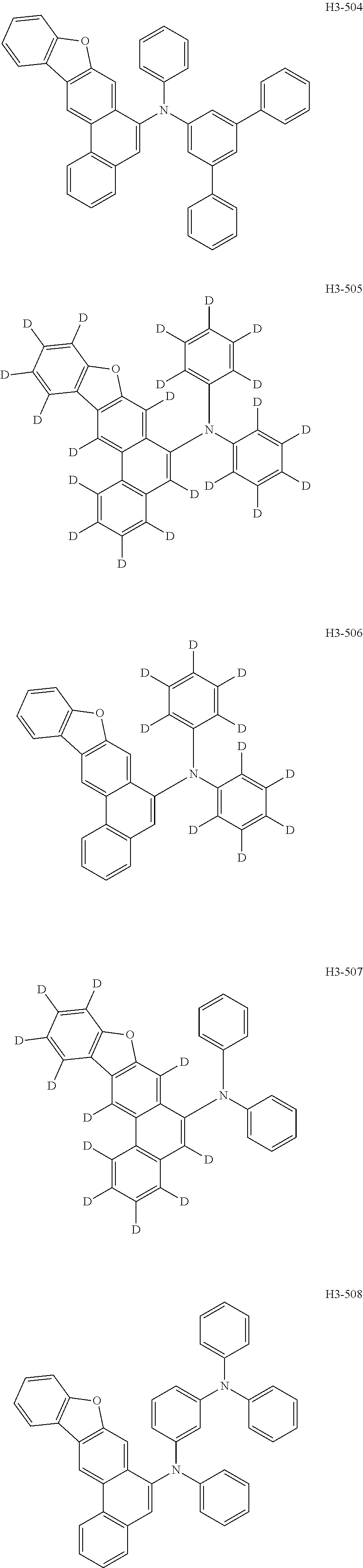

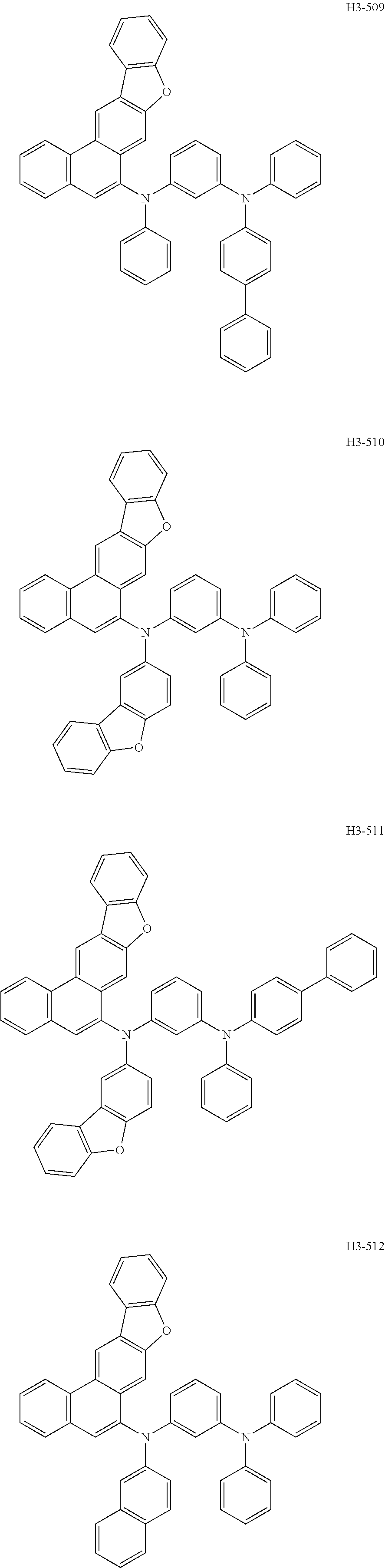 [SMILES: c1ccc(-c2ccc(N(c3ccccc3)c3cccc(N(c4ccccc4)c4cc5ccccc5c5cc6c(cc45)oc4ccccc46)c3)cc2)cc1 ; c1ccc(N(c2ccccc2)c2cccc(N(c3ccc4oc5ccccc5c4c3)c3cc4ccccc4c4cc5c(cc34)oc3ccccc35)c2)cc1 ; c1ccc(-c2ccc(N(c3ccccc3)c3cccc(N(c4ccc5oc6ccccc6c5c4)c4cc5ccccc5c5cc6c(cc45)oc4ccccc46)c3)cc2)cc1 ; c1ccc(N(c2ccccc2)c2cccc(N(c3ccc4ccccc4c3)c3cc4ccccc4c4cc5c(cc34)oc3ccccc35)c2)cc1]

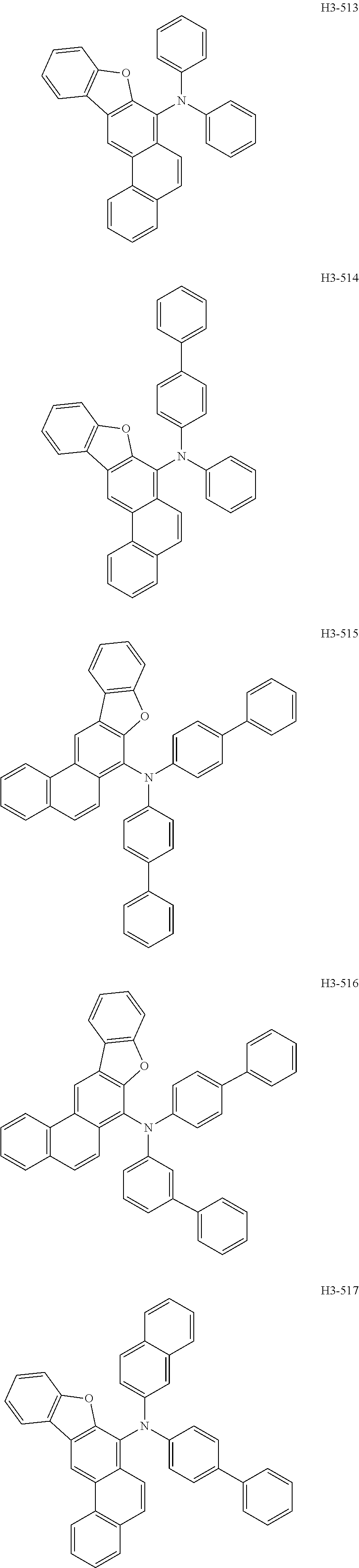 [SMILES: c1ccc(N(c2ccccc2)c2c3ccc4ccccc4c3cc3c2oc2ccccc23)cc1 ; c1ccc(-c2ccc(N(c3ccccc3)c3c4ccc5ccccc5c4cc4c3oc3ccccc34)cc2)cc1 ; c1ccc(-c2ccc(N(c3ccc(-c4ccccc4)cc3)c3c4ccc5ccccc5c4cc4c3oc3ccccc34)cc2)cc1 ; c1ccc(-c2ccc(N(c3cccc(-c4ccccc4)c3)c3c4ccc5ccccc5c4cc4c3oc3ccccc34)cc2)cc1 ; c1ccc(-c2ccc(N(c3ccc4ccccc4c3)c3c4ccc5ccccc5c4cc4c3oc3ccccc34)cc2)cc1]

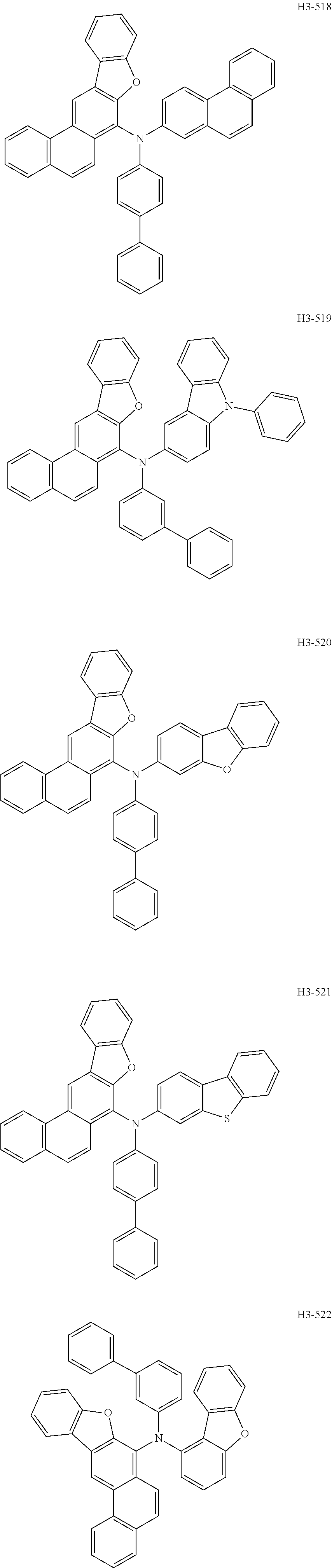 [SMILES: c1ccc(-c2ccc(N(c3ccc4c(ccc5ccccc54)c3)c3c4ccc5ccccc5c4cc4c3oc3ccccc34)cc2)cc1 ; c1ccc(-c2cccc(N(c3ccc4c(c3)c3ccccc3n4-c3ccccc3)c3c4ccc5ccccc5c4cc4c3oc3ccccc34)c2)cc1 ; c1ccc(-c2ccc(N(c3ccc4c(c3)oc3ccccc34)c3c4ccc5ccccc5c4cc4c3oc3ccccc34)cc2)cc1 ; c1ccc(-c2ccc(N(c3ccc4c(c3)sc3ccccc34)c3c4ccc5ccccc5c4cc4c3oc3ccccc34)cc2)cc1 ; c1ccc(-c2cccc(N(c3c4ccc5ccccc5c4cc4c3oc3ccccc34)c3cccc4oc5ccccc5c34)c2)cc1]

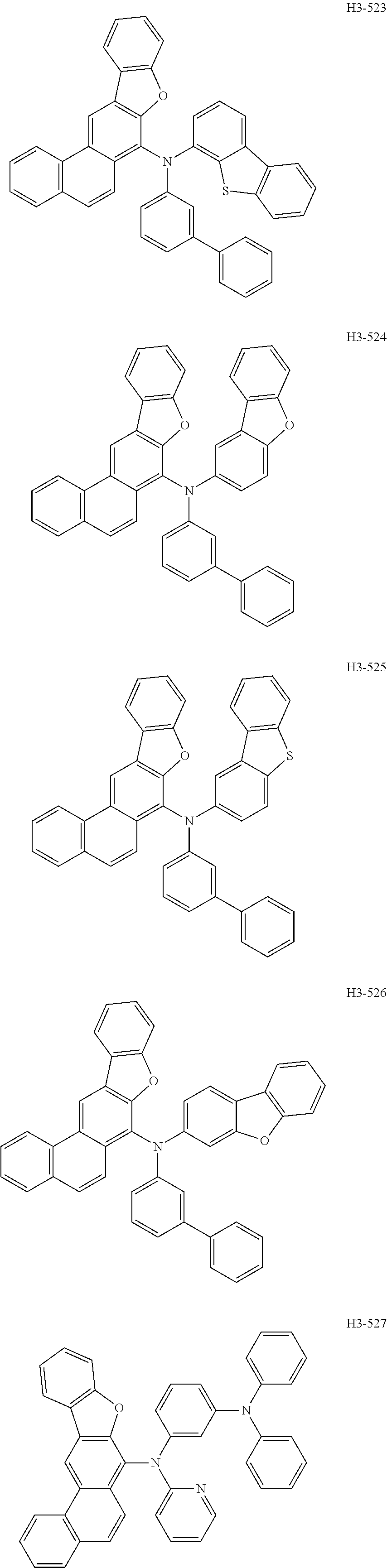 [SMILES: c1ccc(-c2cccc(N(c3c4ccc5ccccc5c4cc4c3oc3ccccc34)c3cccc4c3sc3ccccc34)c2)cc1 ; c1ccc(-c2cccc(N(c3ccc4oc5ccccc5c4c3)c3c4ccc5ccccc5c4cc4c3oc3ccccc34)c2)cc1 ; c1ccc(-c2cccc(N(c3ccc4sc5ccccc5c4c3)c3c4ccc5ccccc5c4cc4c3oc3ccccc34)c2)cc1 ; c1ccc(-c2cccc(N(c3ccc4c(c3)oc3ccccc34)c3c4ccc5ccccc5c4cc4c3oc3ccccc34)c2)cc1 ; c1ccc(N(c2ccccc2)c2cccc(N(c3ccccn3)c3c4ccc5ccccc5c4cc4c3oc3ccccc34)c2)cc1]

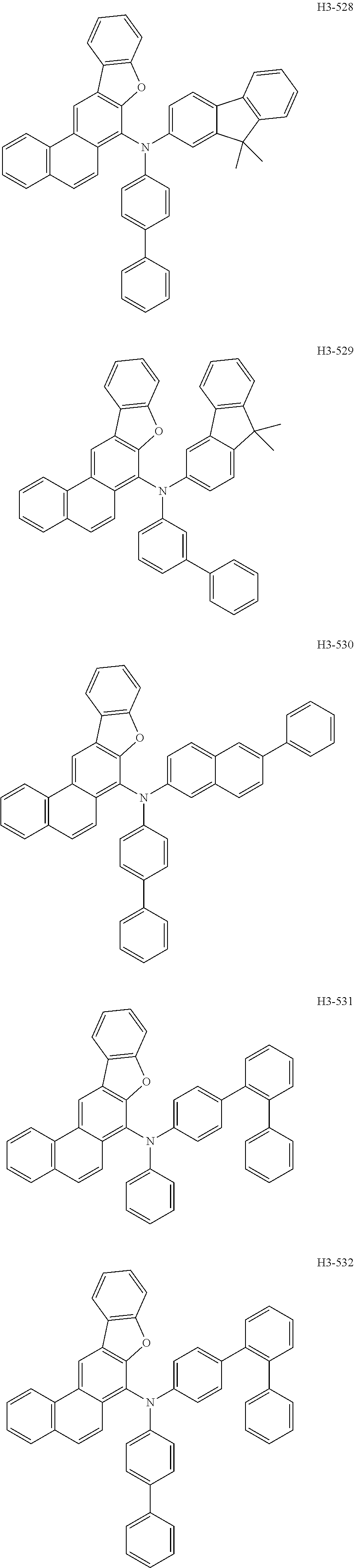 [SMILES: CC1(C)c2ccccc2-c2ccc(N(c3ccc(-c4ccccc4)cc3)c3c4ccc5ccccc5c4cc4c3oc3ccccc34)cc21 ; CC1(C)c2ccccc2-c2cc(N(c3cccc(-c4ccccc4)c3)c3c4ccc5ccccc5c4cc4c3oc3ccccc34)ccc21 ; c1ccc(-c2ccc(N(c3ccc4cc(-c5ccccc5)ccc4c3)c3c4ccc5ccccc5c4cc4c3oc3ccccc34)cc2)cc1 ; c1ccc(-c2ccccc2-c2ccc(N(c3ccccc3)c3c4ccc5ccccc5c4cc4c3oc3ccccc34)cc2)cc1 ; c1ccc(-c2ccc(N(c3ccc(-c4ccccc4-c4ccccc4)cc3)c3c4ccc5ccccc5c4cc4c3oc3ccccc34)cc2)cc1]

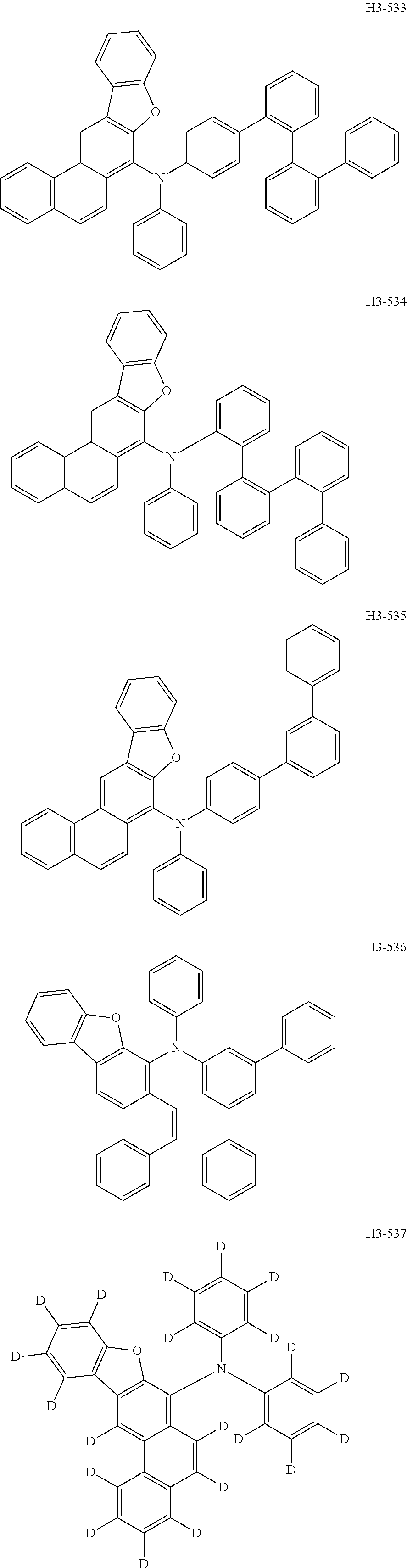

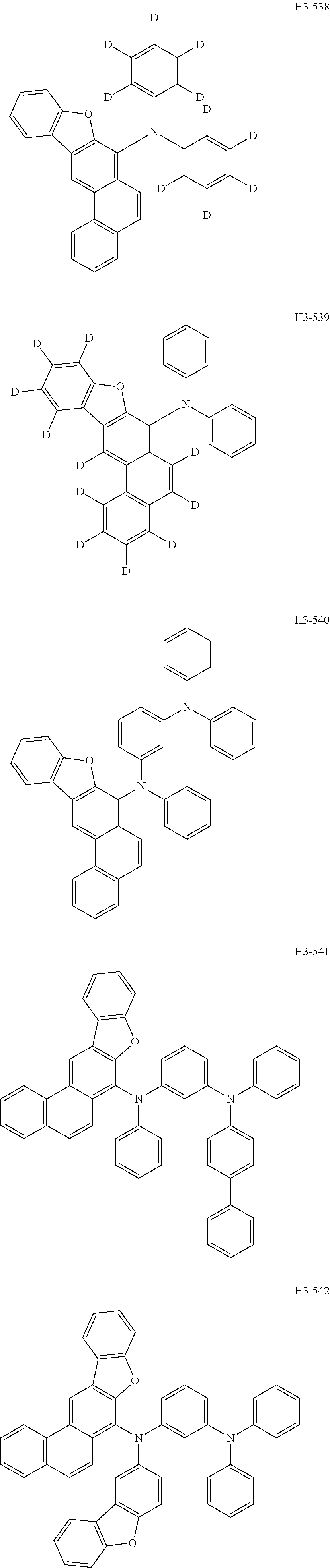 [SMILES: [2H]c1c([2H])c([2H])c(N(c2c([2H])c([2H])c([2H])c([2H])c2[2H])c2c3ccc4ccccc4c3cc3c2oc2ccccc23)c([2H])c1[2H] ; [2H]c1c([2H])c([2H])c2c(oc3c(N(c4ccccc4)c4ccccc4)c4c([2H])c([2H])c5c([2H])c([2H])c([2H])c([2H])c5c4c([2H])c32)c1[2H] ; c1ccc(N(c2ccccc2)c2cccc(N(c3ccccc3)c3c4ccc5ccccc5c4cc4c3oc3ccccc34)c2)cc1 ; c1ccc(-c2ccc(N(c3ccccc3)c3cccc(N(c4ccccc4)c4c5ccc6ccccc6c5cc5c4oc4ccccc45)c3)cc2)cc1 ; c1ccc(N(c2ccccc2)c2cccc(N(c3ccc4oc5ccccc5c4c3)c3c4ccc5ccccc5c4cc4c3oc3ccccc34)c2)cc1]

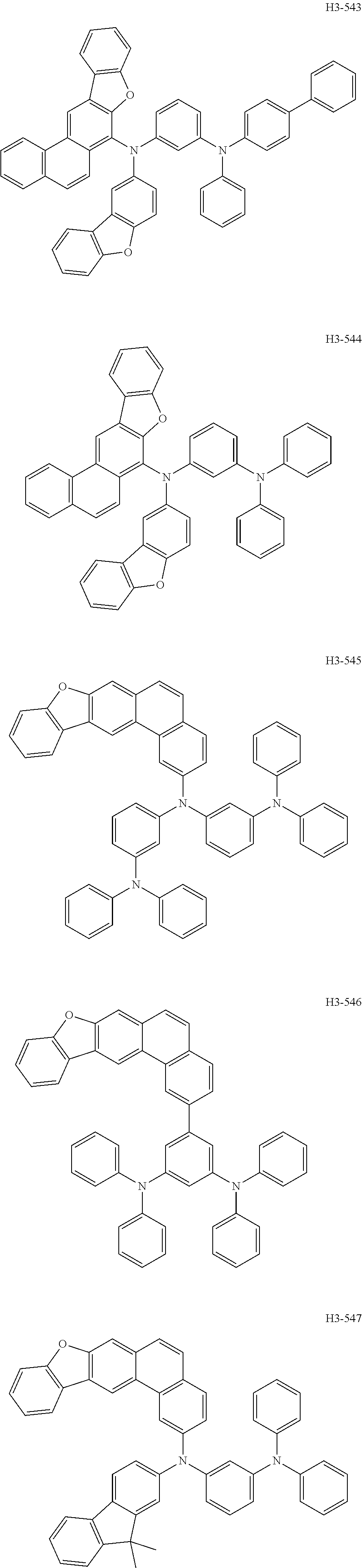 [SMILES: c1ccc(-c2ccc(N(c3ccccc3)c3cccc(N(c4ccc5oc6ccccc6c5c4)c4c5ccc6ccccc6c5cc5c4oc4ccccc45)c3)cc2)cc1 ; c1ccc(N(c2ccccc2)c2cccc(N(c3ccc4oc5ccccc5c4c3)c3c4ccc5ccccc5c4cc4c3oc3ccccc34)c2)cc1 ; c1ccc(N(c2ccccc2)c2cccc(N(c3cccc(N(c4ccccc4)c4ccccc4)c3)c3ccc4ccc5cc6oc7ccccc7c6cc5c4c3)c2)cc1 ; c1ccc(N(c2ccccc2)c2cc(-c3ccc4ccc5cc6oc7ccccc7c6cc5c4c3)cc(N(c3ccccc3)c3ccccc3)c2)cc1 ; CC1(C)c2ccccc2-c2ccc(N(c3cccc(N(c4ccccc4)c4ccccc4)c3)c3ccc4ccc5cc6oc7ccccc7c6cc5c4c3)cc21]

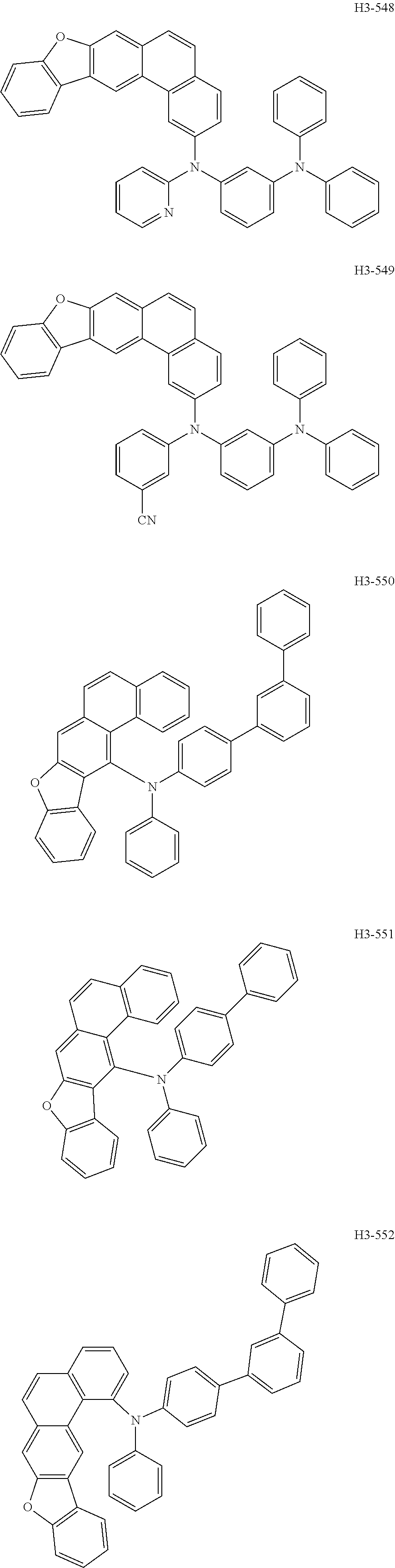 [SMILES: c1ccc(N(c2ccccc2)c2cccc(N(c3ccc4ccc5cc6oc7ccccc7c6cc5c4c3)c3ccccn3)c2)cc1 ; N#Cc1cccc(N(c2cccc(N(c3ccccc3)c3ccccc3)c2)c2ccc3ccc4cc5oc6ccccc6c5cc4c3c2)c1 ; c1ccc(-c2cccc(-c3ccc(N(c4ccccc4)c4c5c(ccc6ccccc65)cc5oc6ccccc6c45)cc3)c2)cc1 ; c1ccc(-c2ccc(N(c3ccccc3)c3c4c(ccc5ccccc54)cc4oc5ccccc5c34)cc2)cc1 ; c1ccc(-c2cccc(-c3ccc(N(c4ccccc4)c4cccc5ccc6cc7oc8ccccc8c7cc6c45)cc3)c2)cc1]

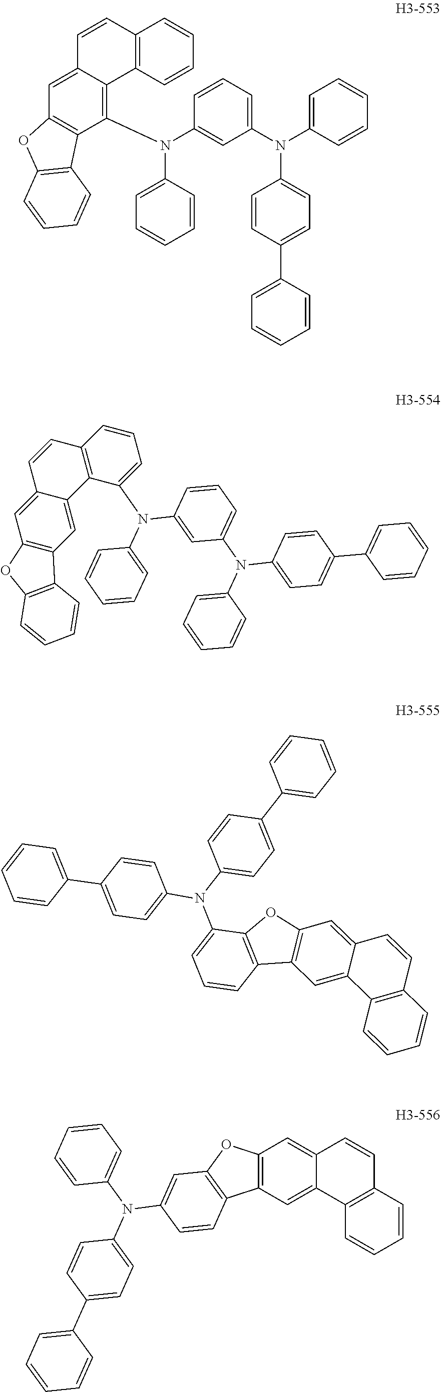 [SMILES: c1ccc(-c2ccc(N(c3ccccc3)c3cccc(N(c4ccccc4)c4c5c(ccc6ccccc65)cc5oc6ccccc6c45)c3)cc2)cc1 ; c1ccc(-c2ccc(N(c3ccccc3)c3cccc(N(c4ccccc4)c4cccc5ccc6cc7oc8ccccc8c7cc6c45)c3)cc2)cc1 ; c1ccc(-c2ccc(N(c3ccc(-c4ccccc4)cc3)c3cccc4c3oc3cc5ccc6ccccc6c5cc34)cc2)cc1 ; c1ccc(-c2ccc(N(c3ccccc3)c3ccc4c(c3)oc3cc5ccc6ccccc6c5cc34)cc2)cc1]

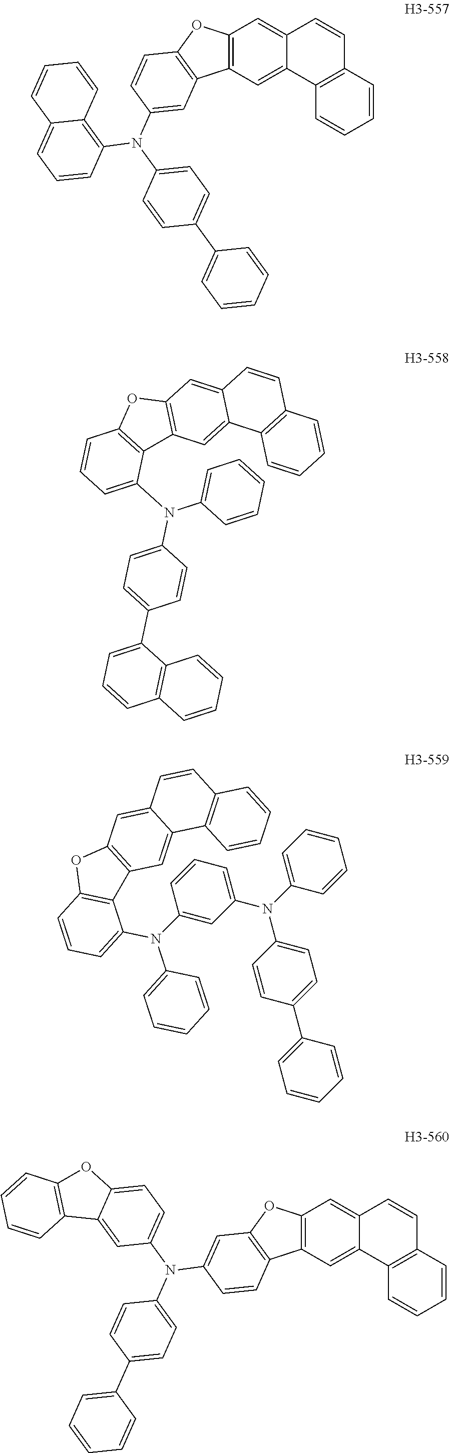 [SMILES: c1ccc(-c2ccc(N(c3ccc4oc5cc6ccc7ccccc7c6cc5c4c3)c3cccc4ccccc34)cc2)cc1 ; c1ccc(N(c2ccc(-c3cccc4ccccc34)cc2)c2cccc3oc4cc5ccc6ccccc6c5cc4c23)cc1 ; c1ccc(-c2ccc(N(c3ccccc3)c3cccc(N(c4ccccc4)c4cccc5oc6cc7ccc8ccccc8c7cc6c45)c3)cc2)cc1 ; c1ccc(-c2ccc(N(c3ccc4c(c3)oc3cc5ccc6ccccc6c5cc34)c3ccc4oc5ccccc5c4c3)cc2)cc1]

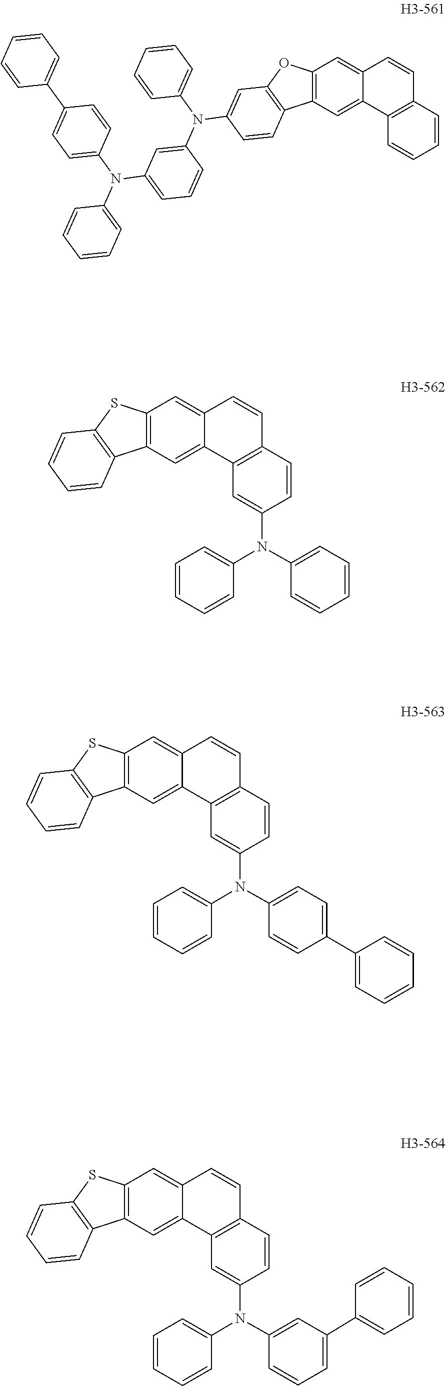 [SMILES: c1ccc(-c2ccc(N(c3ccccc3)c3cccc(N(c4ccccc4)c4ccc5c(c4)oc4cc6ccc7ccccc7c6cc45)c3)cc2)cc1 ; c1ccc(N(c2ccccc2)c2ccc3ccc4cc5sc6ccccc6c5cc4c3c2)cc1 ; c1ccc(-c2ccc(N(c3ccccc3)c3ccc4ccc5cc6sc7ccccc7c6cc5c4c3)cc2)cc1 ; c1ccc(-c2cccc(N(c3ccccc3)c3ccc4ccc5cc6sc7ccccc7c6cc5c4c3)c2)cc1]

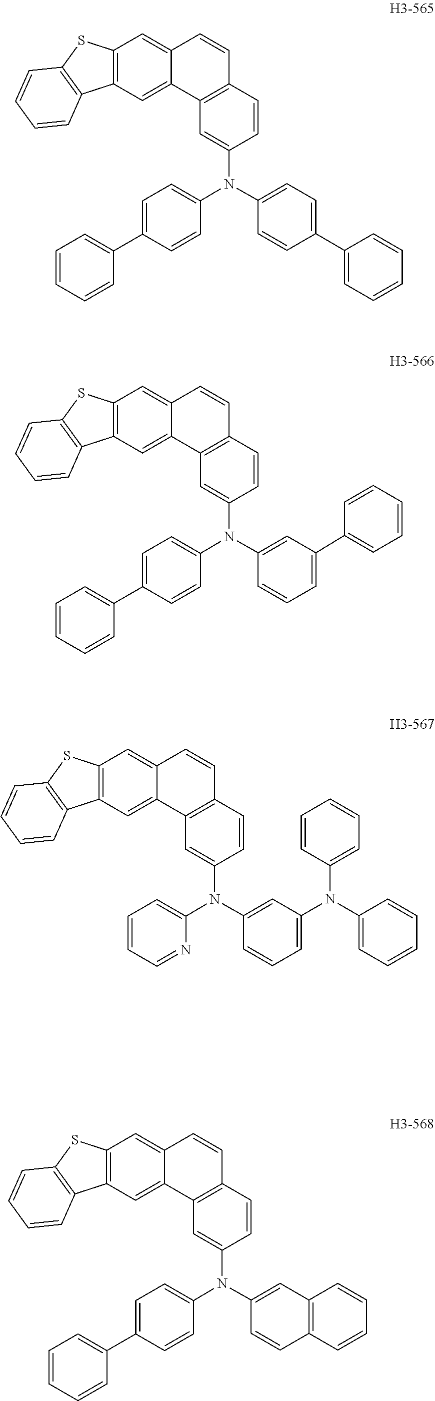 [SMILES: c1ccc(-c2ccc(N(c3ccc(-c4ccccc4)cc3)c3ccc4ccc5cc6sc7ccccc7c6cc5c4c3)cc2)cc1 ; c1ccc(-c2ccc(N(c3cccc(-c4ccccc4)c3)c3ccc4ccc5cc6sc7ccccc7c6cc5c4c3)cc2)cc1 ; c1ccc(N(c2ccccc2)c2cccc(N(c3ccc4ccc5cc6sc7ccccc7c6cc5c4c3)c3ccccn3)c2)cc1 ; c1ccc(-c2ccc(N(c3ccc4ccccc4c3)c3ccc4ccc5cc6sc7ccccc7c6cc5c4c3)cc2)cc1]

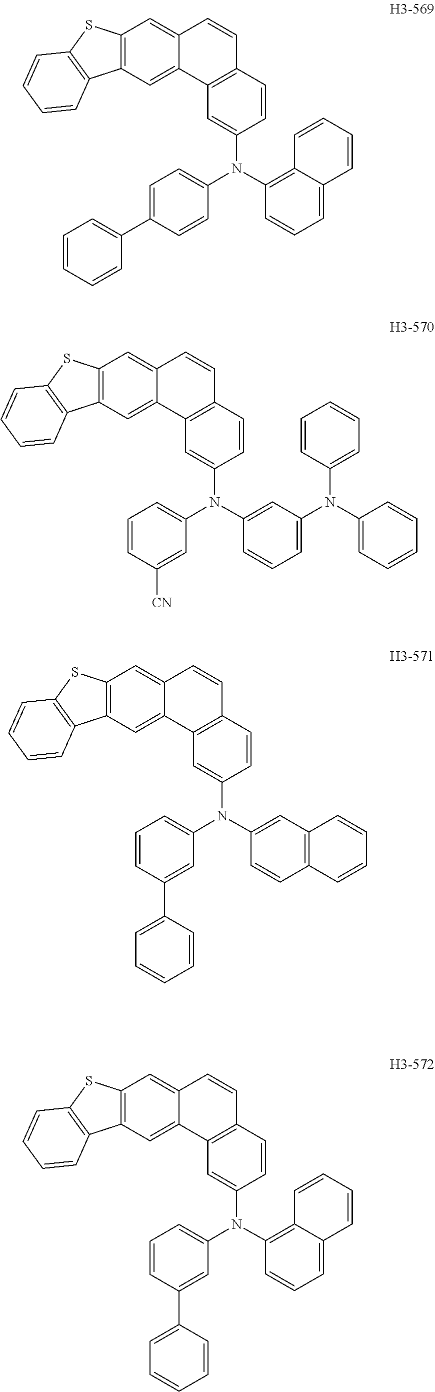 [SMILES: c1ccc(-c2ccc(N(c3ccc4ccc5cc6sc7ccccc7c6cc5c4c3)c3cccc4ccccc34)cc2)cc1 ; N#Cc1cccc(N(c2cccc(N(c3ccccc3)c3ccccc3)c2)c2ccc3ccc4cc5sc6ccccc6c5cc4c3c2)c1 ; c1ccc(-c2cccc(N(c3ccc4ccccc4c3)c3ccc4ccc5cc6sc7ccccc7c6cc5c4c3)c2)cc1 ; c1ccc(-c2cccc(N(c3ccc4ccc5cc6sc7ccccc7c6cc5c4c3)c3cccc4ccccc34)c2)cc1]

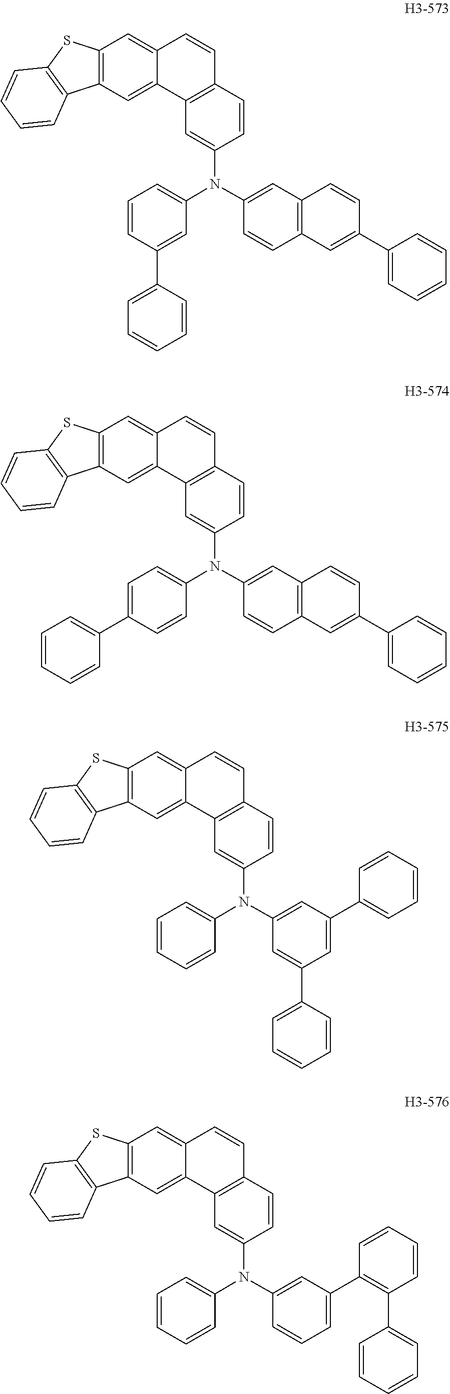 [SMILES: c1ccc(-c2cccc(N(c3ccc4cc(-c5ccccc5)ccc4c3)c3ccc4ccc5cc6sc7ccccc7c6cc5c4c3)c2)cc1 ; c1ccc(-c2ccc(N(c3ccc4cc(-c5ccccc5)ccc4c3)c3ccc4ccc5cc6sc7ccccc7c6cc5c4c3)cc2)cc1 ; c1ccc(-c2cc(-c3ccccc3)cc(N(c3ccccc3)c3ccc4ccc5cc6sc7ccccc7c6cc5c4c3)c2)cc1 ; c1ccc(-c2ccccc2-c2cccc(N(c3ccccc3)c3ccc4ccc5cc6sc7ccccc7c6cc5c4c3)c2)cc1]

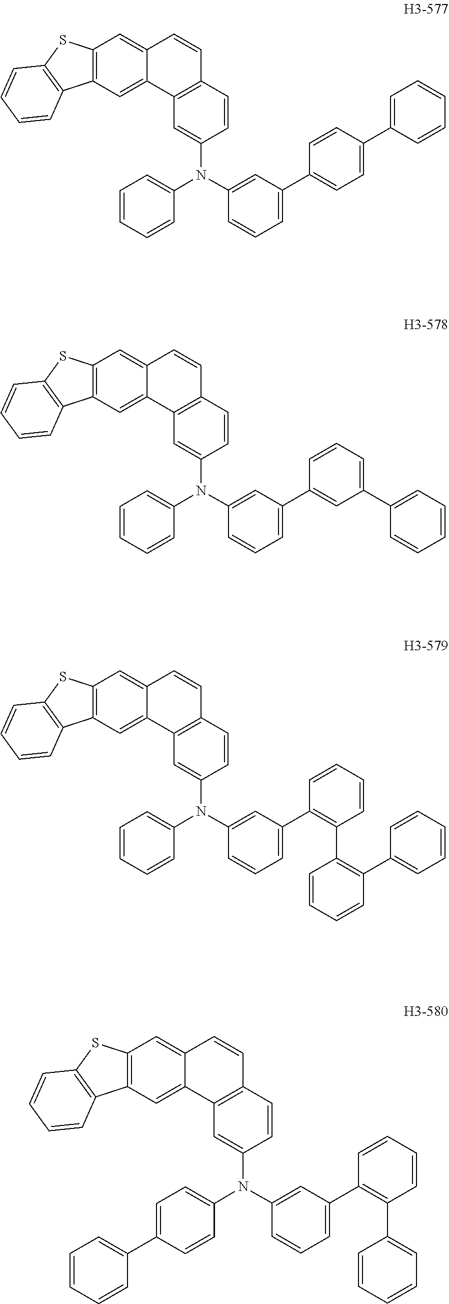 [SMILES: c1ccc(-c2ccc(-c3cccc(N(c4ccccc4)c4ccc5ccc6cc7sc8ccccc8c7cc6c5c4)c3)cc2)cc1 ; c1ccc(-c2cccc(-c3cccc(N(c4ccccc4)c4ccc5ccc6cc7sc8ccccc8c7cc6c5c4)c3)c2)cc1 ; c1ccc(-c2ccccc2-c2ccccc2-c2cccc(N(c3ccccc3)c3ccc4ccc5cc6sc7ccccc7c6cc5c4c3)c2)cc1 ; c1ccc(-c2ccc(N(c3cccc(-c4ccccc4-c4ccccc4)c3)c3ccc4ccc5cc6sc7ccccc7c6cc5c4c3)cc2)cc1]

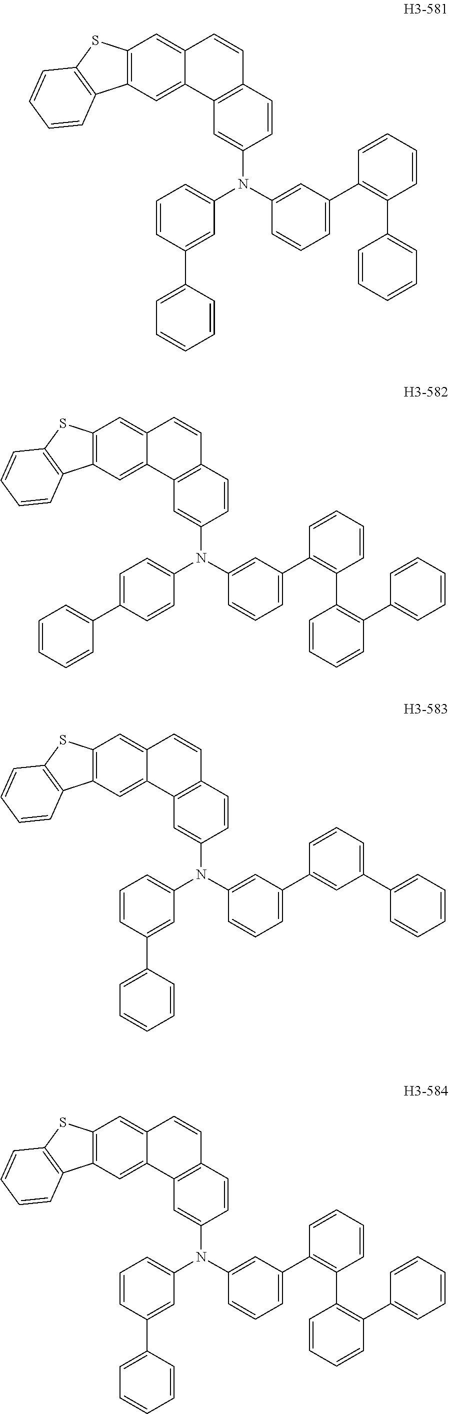 [SMILES: c1ccc(-c2cccc(N(c3cccc(-c4ccccc4-c4ccccc4)c3)c3ccc4ccc5cc6sc7ccccc7c6cc5c4c3)c2)cc1 ; c1ccc(-c2ccc(N(c3cccc(-c4ccccc4-c4ccccc4-c4ccccc4)c3)c3ccc4ccc5cc6sc7ccccc7c6cc5c4c3)cc2)cc1 ; c1ccc(-c2cccc(-c3cccc(N(c4cccc(-c5ccccc5)c4)c4ccc5ccc6cc7sc8ccccc8c7cc6c5c4)c3)c2)cc1 ; c1ccc(-c2cccc(N(c3cccc(-c4ccccc4-c4ccccc4-c4ccccc4)c3)c3ccc4ccc5cc6sc7ccccc7c6cc5c4c3)c2)cc1]

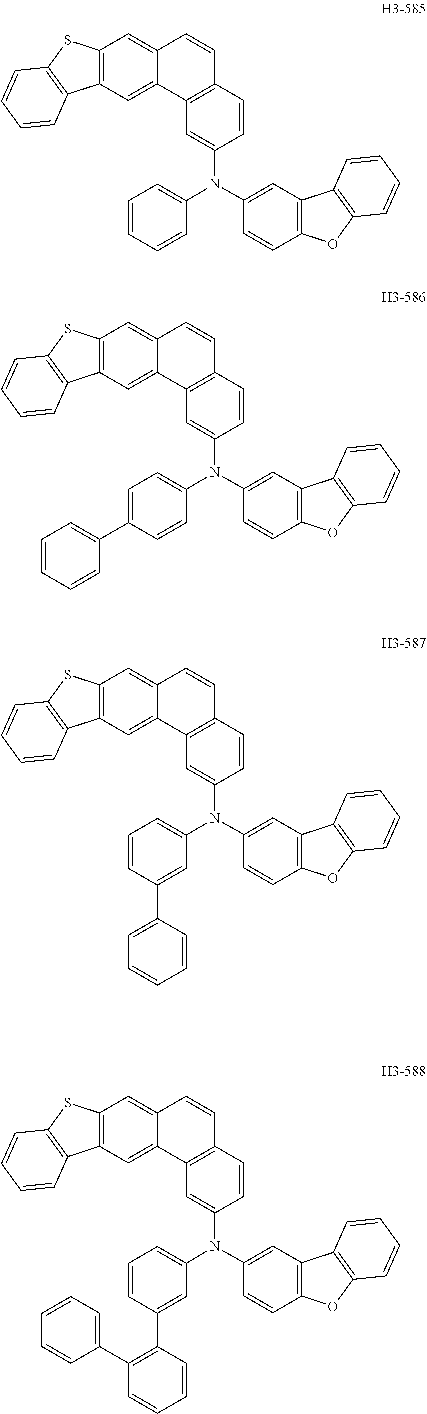 [SMILES: c1ccc(N(c2ccc3ccc4cc5sc6ccccc6c5cc4c3c2)c2ccc3oc4ccccc4c3c2)cc1 ; c1ccc(-c2ccc(N(c3ccc4ccc5cc6sc7ccccc7c6cc5c4c3)c3ccc4oc5ccccc5c4c3)cc2)cc1 ; c1ccc(-c2cccc(N(c3ccc4ccc5cc6sc7ccccc7c6cc5c4c3)c3ccc4oc5ccccc5c4c3)c2)cc1 ; c1ccc(-c2ccccc2-c2cccc(N(c3ccc4ccc5cc6sc7ccccc7c6cc5c4c3)c3ccc4oc5ccccc5c4c3)c2)cc1]

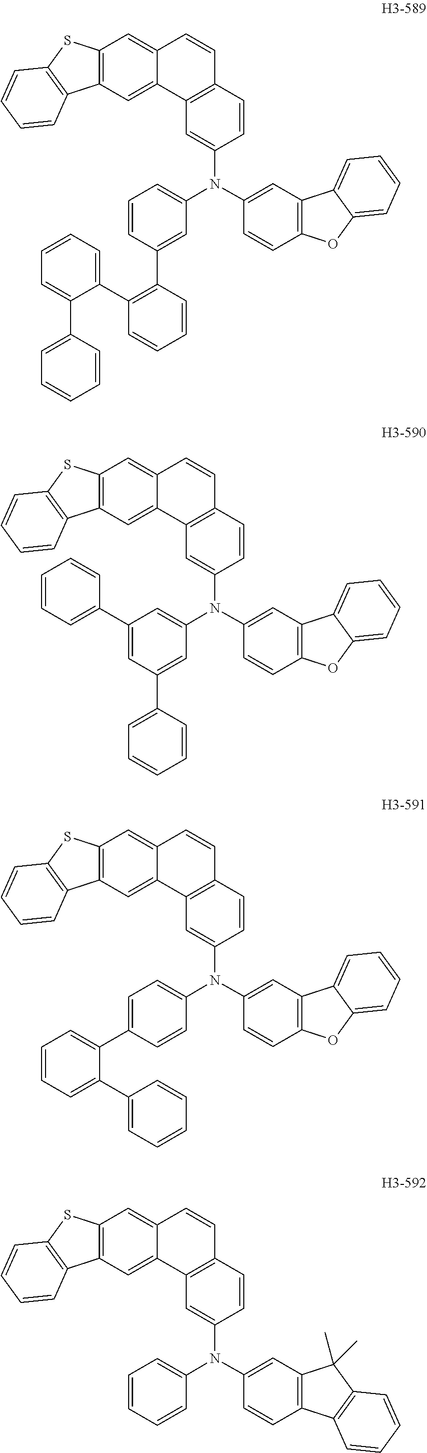 [SMILES: c1ccc(-c2ccccc2-c2ccccc2-c2cccc(N(c3ccc4ccc5cc6sc7ccccc7c6cc5c4c3)c3ccc4oc5ccccc5c4c3)c2)cc1 ; c1ccc(-c2cc(-c3ccccc3)cc(N(c3ccc4ccc5cc6sc7ccccc7c6cc5c4c3)c3ccc4oc5ccccc5c4c3)c2)cc1 ; c1ccc(-c2ccccc2-c2ccc(N(c3ccc4ccc5cc6sc7ccccc7c6cc5c4c3)c3ccc4oc5ccccc5c4c3)cc2)cc1 ; CC1(C)c2ccccc2-c2ccc(N(c3ccccc3)c3ccc4ccc5cc6sc7ccccc7c6cc5c4c3)cc21]

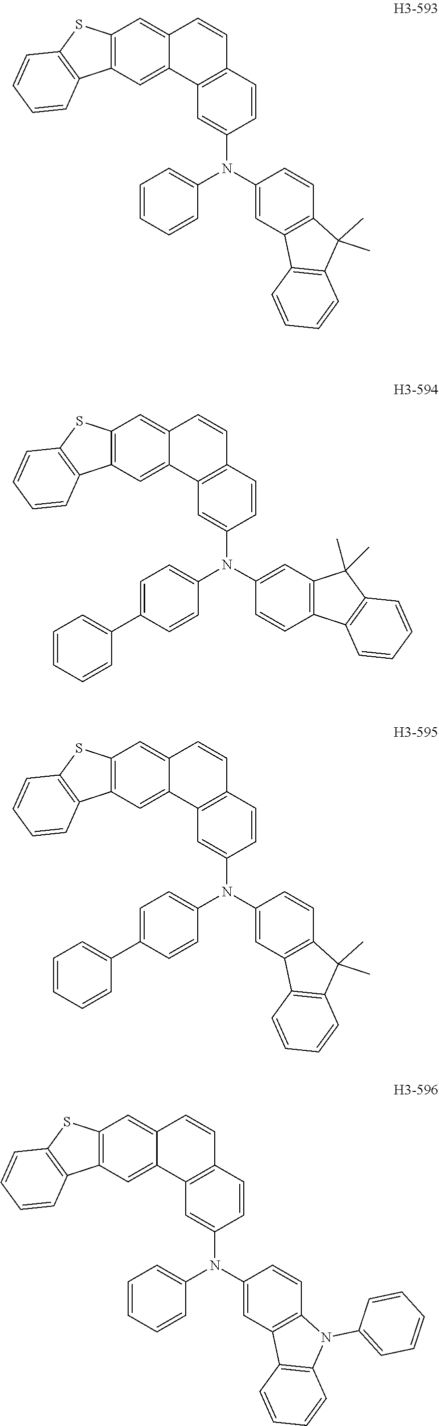 [SMILES: CC1(C)c2ccccc2-c2cc(N(c3ccccc3)c3ccc4ccc5cc6sc7ccccc7c6cc5c4c3)ccc21 ; CC1(C)c2ccccc2-c2ccc(N(c3ccc(-c4ccccc4)cc3)c3ccc4ccc5cc6sc7ccccc7c6cc5c4c3)cc21 ; CC1(C)c2ccccc2-c2cc(N(c3ccc(-c4ccccc4)cc3)c3ccc4ccc5cc6sc7ccccc7c6cc5c4c3)ccc21 ; c1ccc(N(c2ccc3ccc4cc5sc6ccccc6c5cc4c3c2)c2ccc3c(c2)c2ccccc2n3-c2ccccc2)cc1]

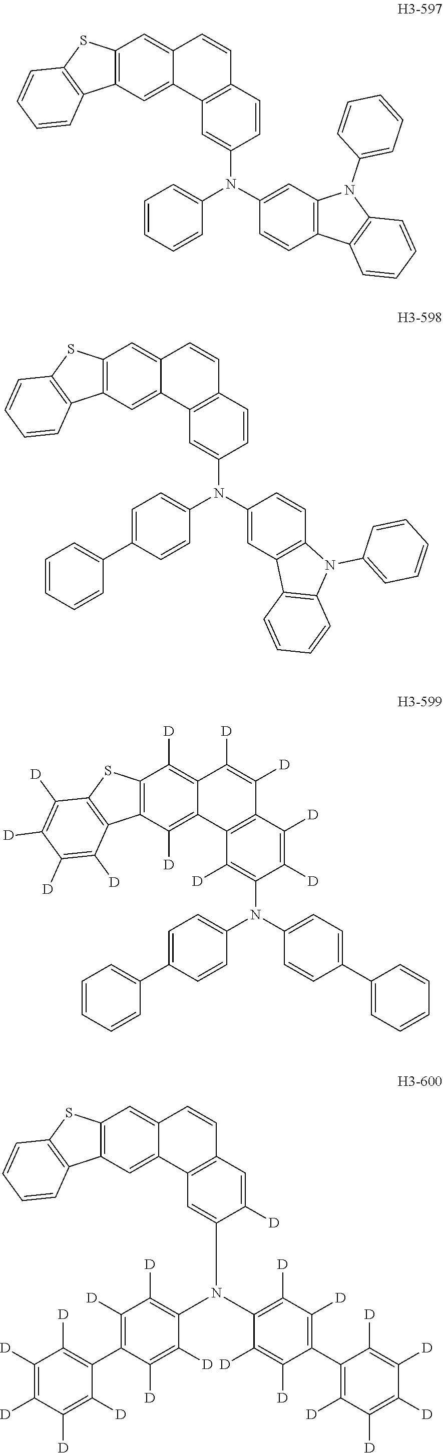 [SMILES: c1ccc(N(c2ccc3ccc4cc5sc6ccccc6c5cc4c3c2)c2ccc3c4ccccc4n(-c4ccccc4)c3c2)cc1 ; c1ccc(-c2ccc(N(c3ccc4ccc5cc6sc7ccccc7c6cc5c4c3)c3ccc4c(c3)c3ccccc3n4-c3ccccc3)cc2)cc1 ; [2H]c1c([2H])c([2H])c2c(sc3c([2H])c4c([2H])c([2H])c5c([2H])c([2H])c(N(c6ccc(-c7ccccc7)cc6)c6ccc(-c7ccccc7)cc6)c([2H])c5c4c([2H])c32)c1[2H] ; [2H]c1cc2ccc3cc4sc5ccccc5c4cc3c2cc1N(c1c([2H])c([2H])c(-c2c([2H])c([2H])c([2H])c([2H])c2[2H])c([2H])c1[2H])c1c([2H])c([2H])c(-c2c([2H])c([2H])c([2H])c([2H])c2[2H])c([2H])c1[2H]]

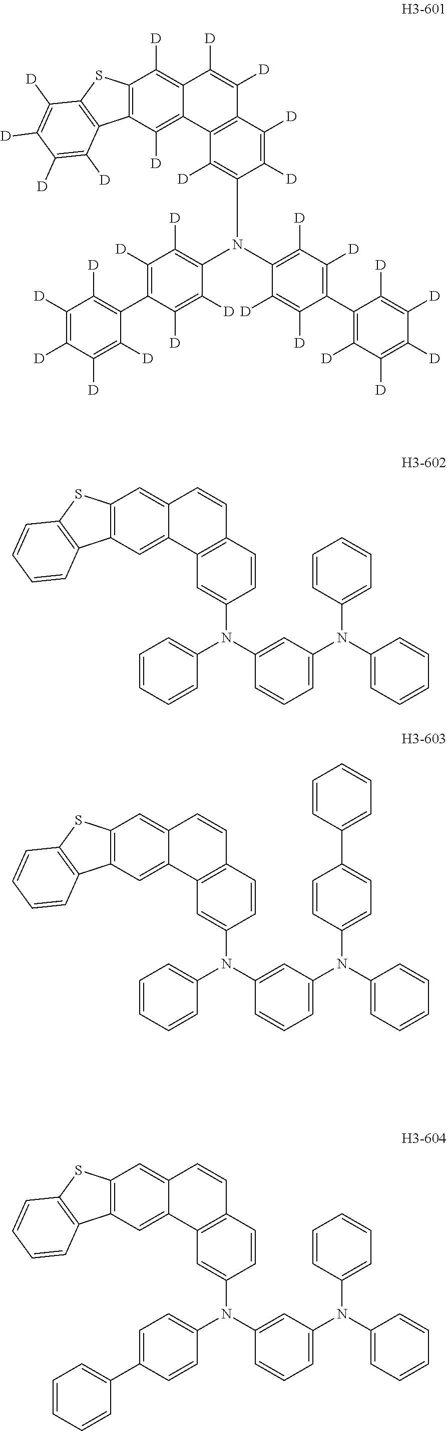 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c4c([2H])c([2H])c5c([2H])c6sc7c([2H])c([2H])c([2H])c([2H])c7c6c([2H])c5c4c3[2H])c([2H])c2[2H])c([2H])c1[2H] ; c1ccc(N(c2ccccc2)c2cccc(N(c3ccccc3)c3ccc4ccc5cc6sc7ccccc7c6cc5c4c3)c2)cc1 ; c1ccc(-c2ccc(N(c3ccccc3)c3cccc(N(c4ccccc4)c4ccc5ccc6cc7sc8ccccc8c7cc6c5c4)c3)cc2)cc1 ; c1ccc(-c2ccc(N(c3cccc(N(c4ccccc4)c4ccccc4)c3)c3ccc4ccc5cc6sc7ccccc7c6cc5c4c3)cc2)cc1]

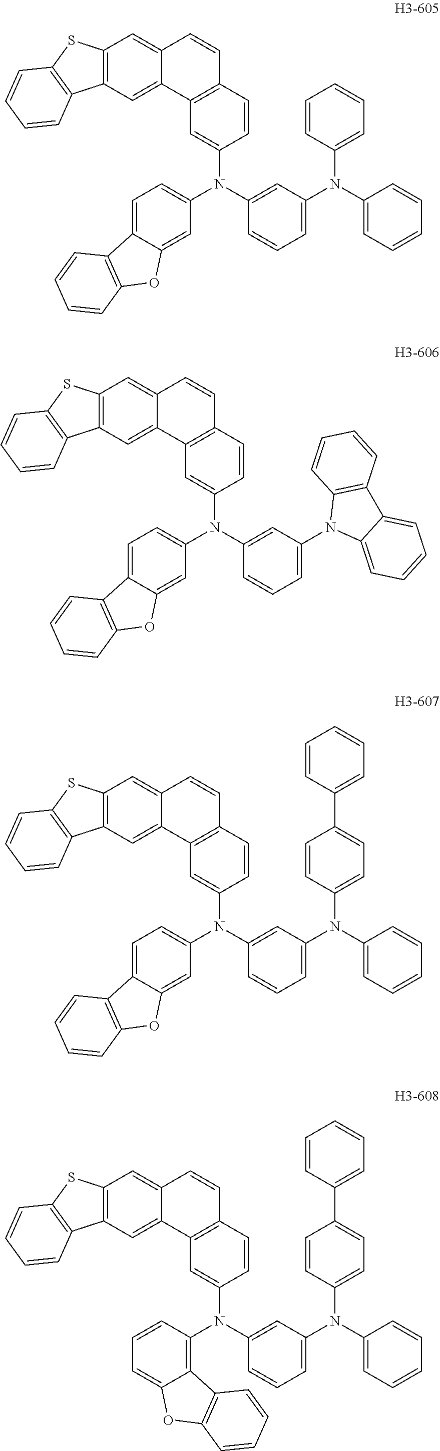 [SMILES: c1ccc(N(c2ccccc2)c2cccc(N(c3ccc4c(c3)oc3ccccc34)c3ccc4ccc5cc6sc7ccccc7c6cc5c4c3)c2)cc1 ; c1cc(N(c2ccc3c(c2)oc2ccccc23)c2ccc3ccc4cc5sc6ccccc6c5cc4c3c2)cc(-n2c3ccccc3c3ccccc32)c1 ; c1ccc(-c2ccc(N(c3ccccc3)c3cccc(N(c4ccc5c(c4)oc4ccccc45)c4ccc5ccc6cc7sc8ccccc8c7cc6c5c4)c3)cc2)cc1 ; c1ccc(-c2ccc(N(c3ccccc3)c3cccc(N(c4ccc5ccc6cc7sc8ccccc8c7cc6c5c4)c4cccc5oc6ccccc6c45)c3)cc2)cc1]

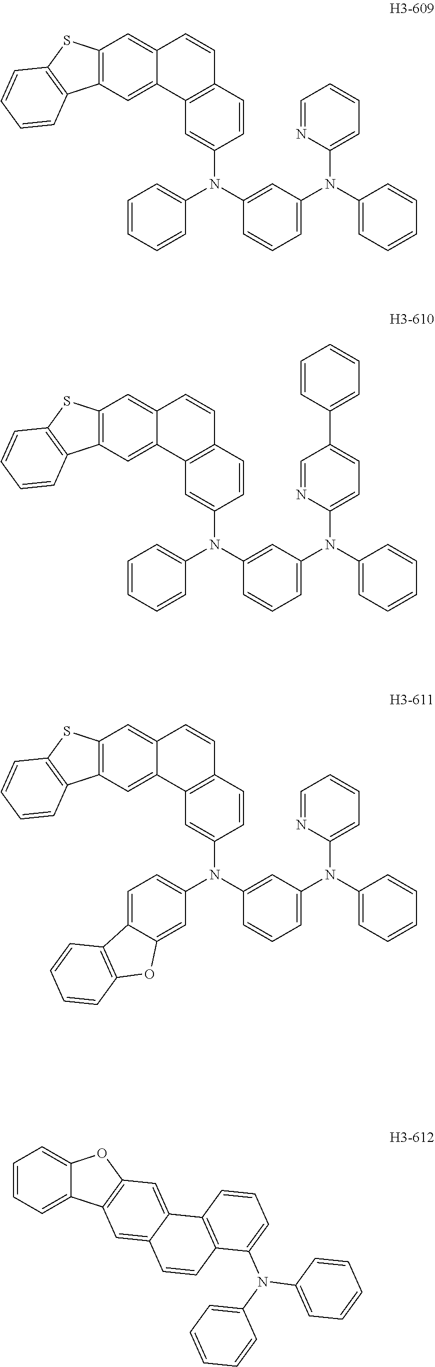 [SMILES: c1ccc(N(c2cccc(N(c3ccccc3)c3ccccn3)c2)c2ccc3ccc4cc5sc6ccccc6c5cc4c3c2)cc1 ; c1ccc(-c2ccc(N(c3ccccc3)c3cccc(N(c4ccccc4)c4ccc5ccc6cc7sc8ccccc8c7cc6c5c4)c3)nc2)cc1 ; c1ccc(N(c2cccc(N(c3ccc4c(c3)oc3ccccc34)c3ccc4ccc5cc6sc7ccccc7c6cc5c4c3)c2)c2ccccn2)cc1 ; c1ccc(N(c2ccccc2)c2cccc3c2ccc2cc4c(cc23)oc2ccccc24)cc1]

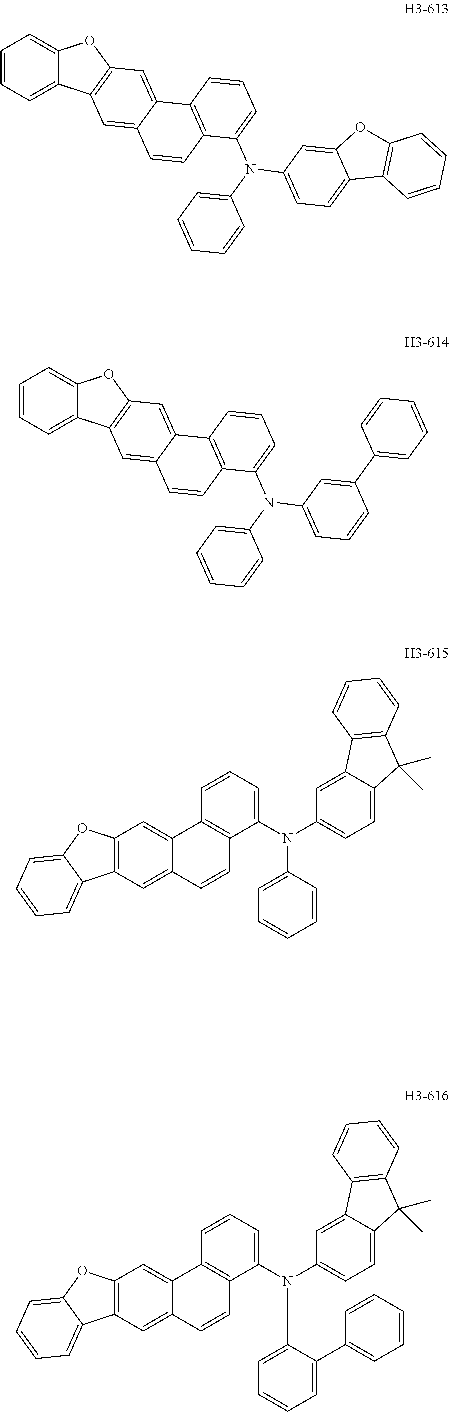 [SMILES: c1ccc(N(c2ccc3c(c2)oc2ccccc23)c2cccc3c2ccc2cc4c(cc23)oc2ccccc24)cc1 ; c1ccc(-c2cccc(N(c3ccccc3)c3cccc4c3ccc3cc5c(cc34)oc3ccccc35)c2)cc1 ; CC1(C)c2ccccc2-c2cc(N(c3ccccc3)c3cccc4c3ccc3cc5c(cc34)oc3ccccc35)ccc21 ; CC1(C)c2ccccc2-c2cc(N(c3ccccc3-c3ccccc3)c3cccc4c3ccc3cc5c(cc34)oc3ccccc35)ccc21]

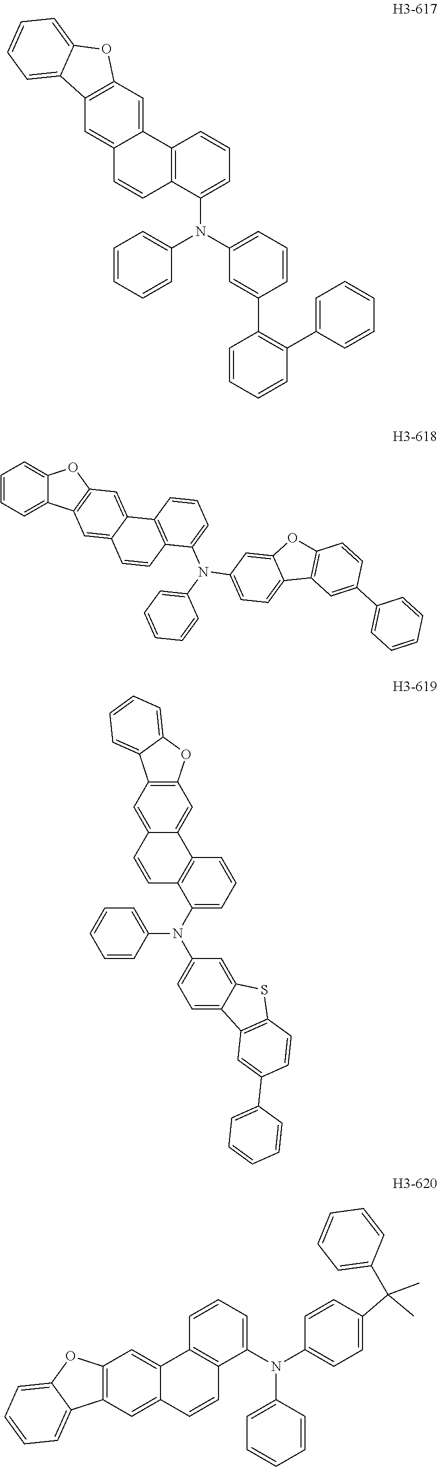 [SMILES: c1ccc(-c2ccccc2-c2cccc(N(c3ccccc3)c3cccc4c3ccc3cc5c(cc34)oc3ccccc35)c2)cc1 ; c1ccc(-c2ccc3oc4cc(N(c5ccccc5)c5cccc6c5ccc5cc7c(cc56)oc5ccccc57)ccc4c3c2)cc1 ; c1ccc(-c2ccc3sc4cc(N(c5ccccc5)c5cccc6c5ccc5cc7c(cc56)oc5ccccc57)ccc4c3c2)cc1 ; CC(C)(c1ccccc1)c1ccc(N(c2ccccc2)c2cccc3c2ccc2cc4c(cc23)oc2ccccc24)cc1]

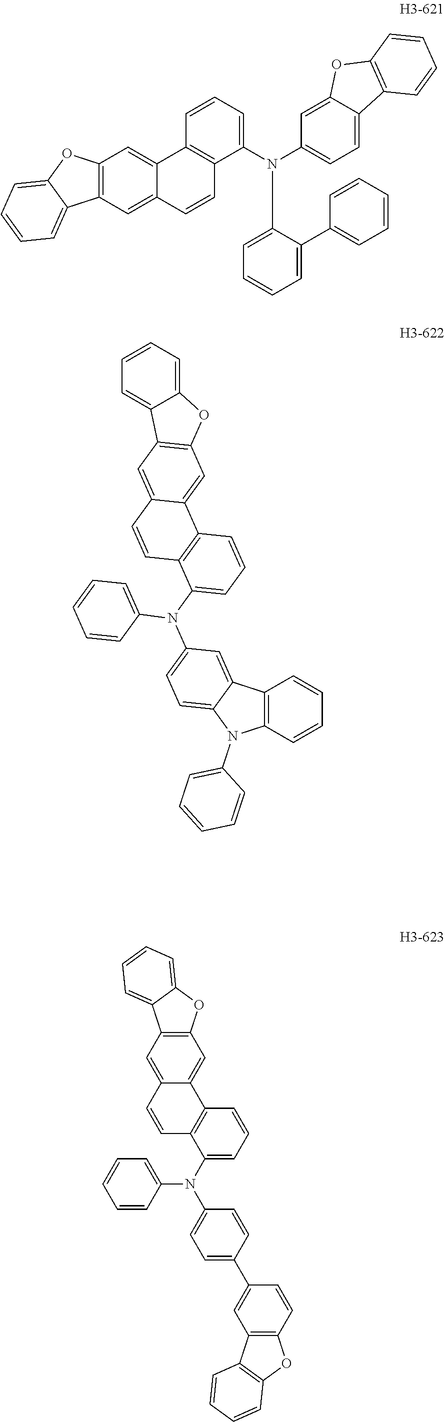 [SMILES: c1ccc(-c2ccccc2N(c2ccc3c(c2)oc2ccccc23)c2cccc3c2ccc2cc4c(cc23)oc2ccccc24)cc1 ; c1ccc(N(c2ccc3c(c2)c2ccccc2n3-c2ccccc2)c2cccc3c2ccc2cc4c(cc23)oc2ccccc24)cc1 ; c1ccc(N(c2ccc(-c3ccc4oc5ccccc5c4c3)cc2)c2cccc3c2ccc2cc4c(cc23)oc2ccccc24)cc1]

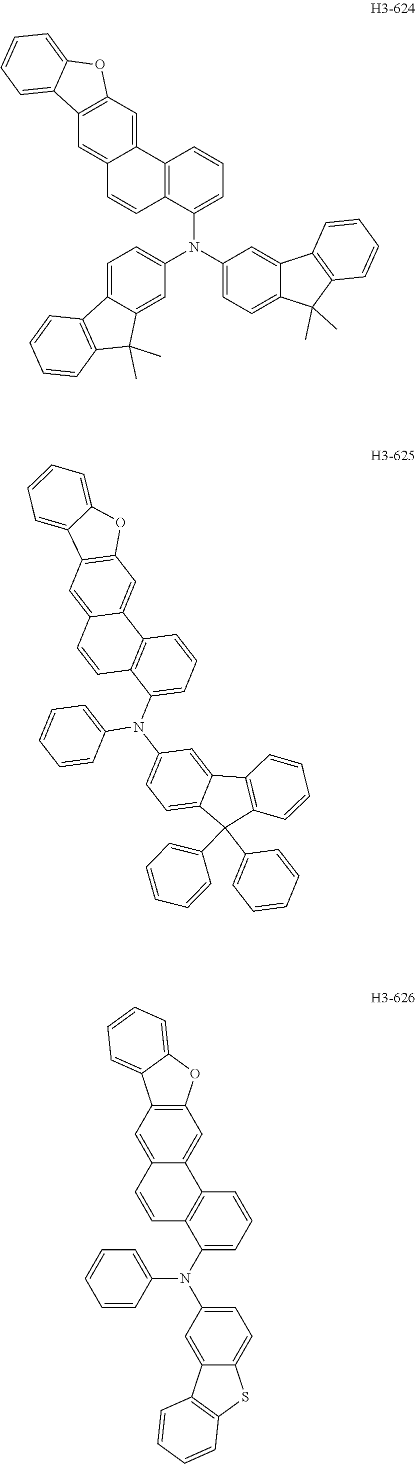 [SMILES: CC1(C)c2ccccc2-c2cc(N(c3ccc4c(c3)C(C)(C)c3ccccc3-4)c3cccc4c3ccc3cc5c(cc34)oc3ccccc35)ccc21 ; c1ccc(N(c2ccc3c(c2)-c2ccccc2C3(c2ccccc2)c2ccccc2)c2cccc3c2ccc2cc4c(cc23)oc2ccccc24)cc1 ; c1ccc(N(c2ccc3sc4ccccc4c3c2)c2cccc3c2ccc2cc4c(cc23)oc2ccccc24)cc1]

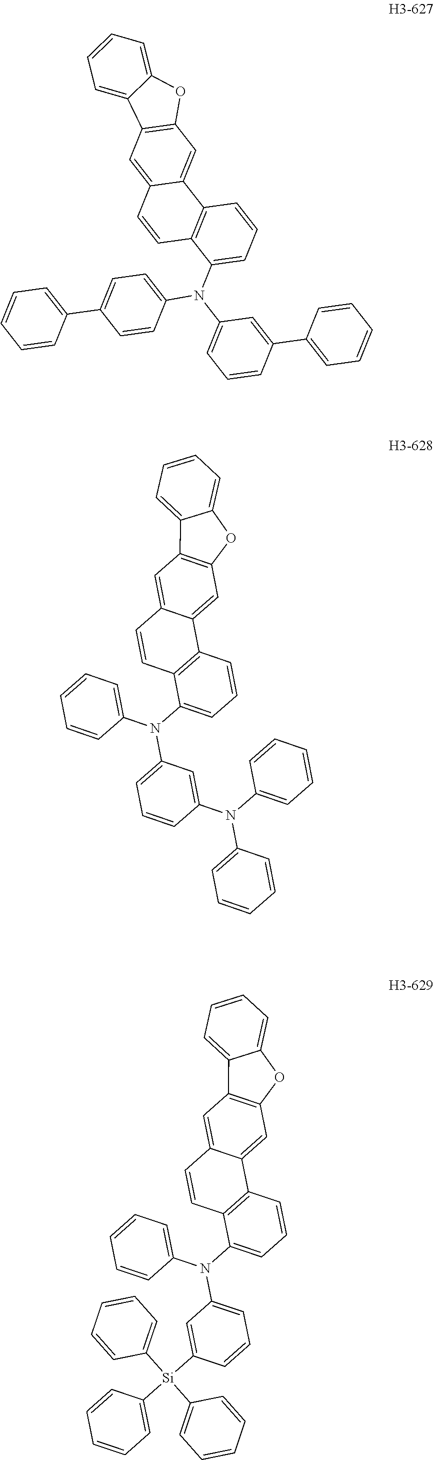 [SMILES: c1ccc(-c2ccc(N(c3cccc(-c4ccccc4)c3)c3cccc4c3ccc3cc5c(cc34)oc3ccccc35)cc2)cc1 ; c1ccc(N(c2ccccc2)c2cccc(N(c3ccccc3)c3cccc4c3ccc3cc5c(cc34)oc3ccccc35)c2)cc1 ; c1ccc(N(c2cccc([Si](c3ccccc3)(c3ccccc3)c3ccccc3)c2)c2cccc3c2ccc2cc4c(cc23)oc2ccccc24)cc1]

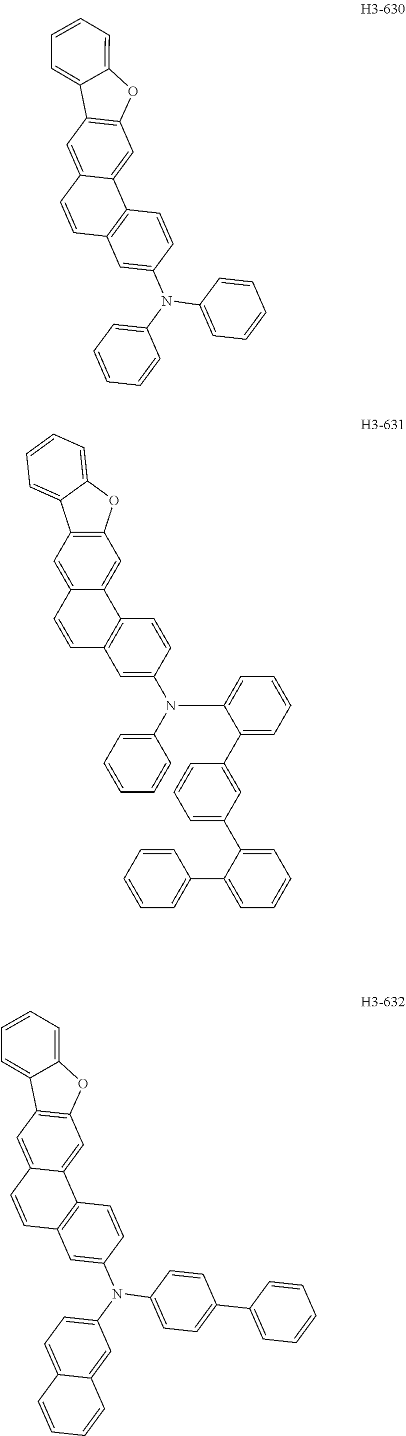 [SMILES: c1ccc(N(c2ccccc2)c2ccc3c(ccc4cc5c(cc43)oc3ccccc35)c2)cc1 ; c1ccc(-c2ccccc2-c2cccc(-c3ccccc3N(c3ccccc3)c3ccc4c(ccc5cc6c(cc54)oc4ccccc46)c3)c2)cc1 ; c1ccc(-c2ccc(N(c3ccc4ccccc4c3)c3ccc4c(ccc5cc6c(cc54)oc4ccccc46)c3)cc2)cc1]

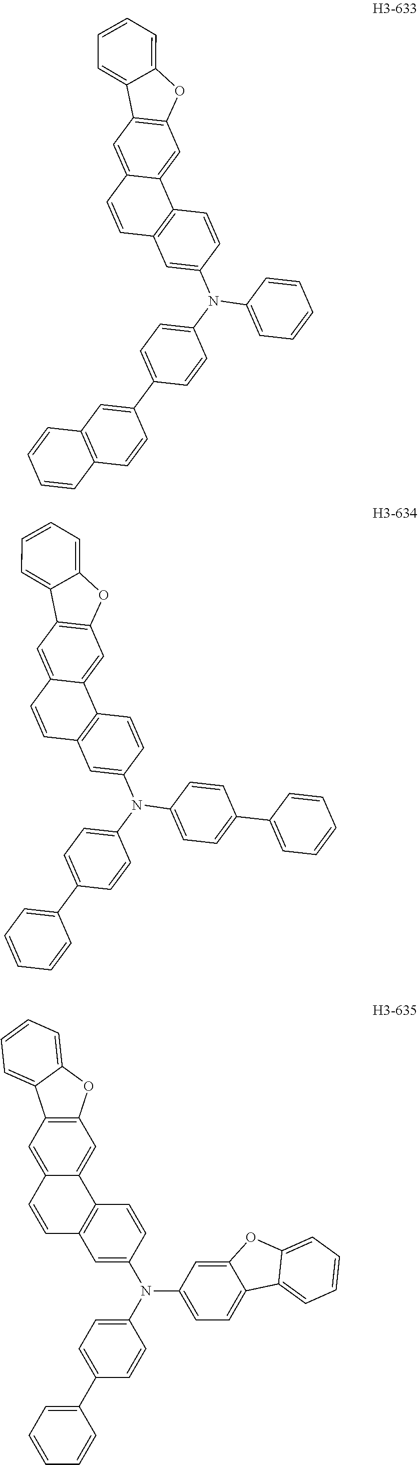

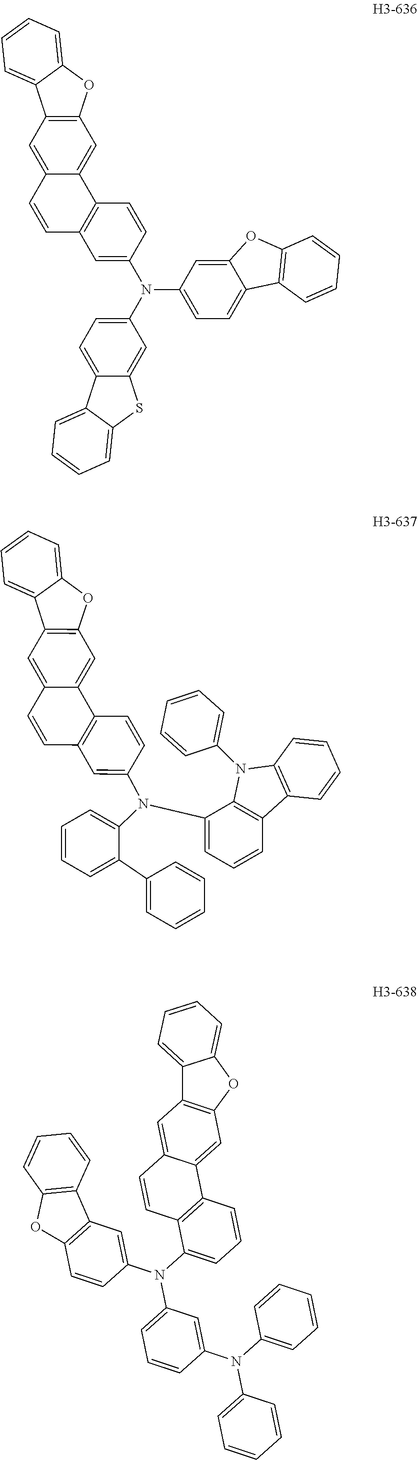 [SMILES: c1ccc2c(c1)oc1cc(N(c3ccc4c(ccc5cc6c(cc54)oc4ccccc46)c3)c3ccc4c(c3)sc3ccccc34)ccc12 ; c1ccc(-c2ccccc2N(c2ccc3c(ccc4cc5c(cc43)oc3ccccc35)c2)c2cccc3c4ccccc4n(-c4ccccc4)c23)cc1 ; c1ccc(N(c2ccccc2)c2cccc(N(c3ccc4oc5ccccc5c4c3)c3cccc4c3ccc3cc5c(cc34)oc3ccccc35)c2)cc1]

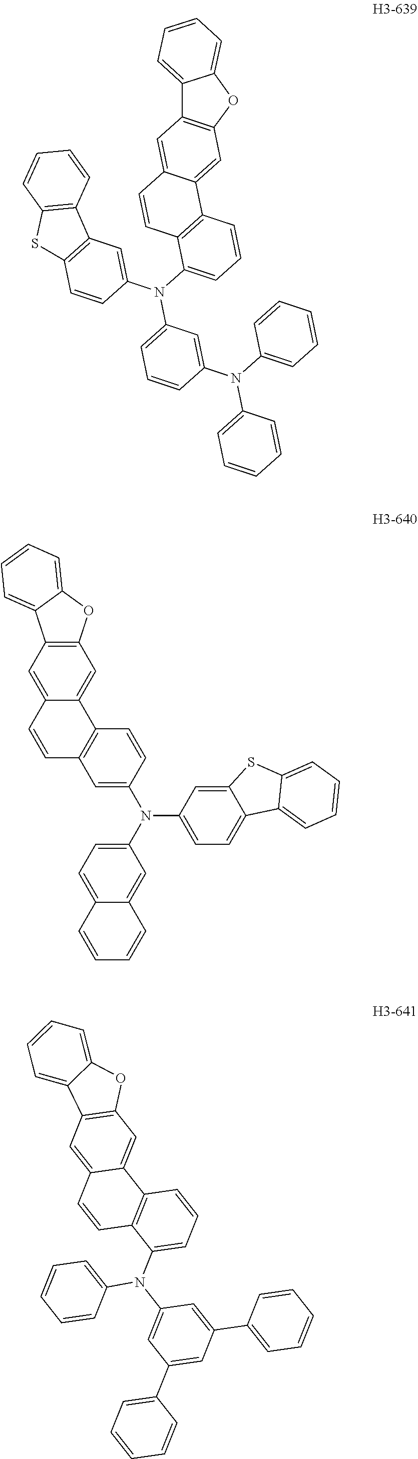 [SMILES: c1ccc(N(c2ccccc2)c2cccc(N(c3ccc4sc5ccccc5c4c3)c3cccc4c3ccc3cc5c(cc34)oc3ccccc35)c2)cc1 ; c1ccc2cc(N(c3ccc4c(ccc5cc6c(cc54)oc4ccccc46)c3)c3ccc4c(c3)sc3ccccc34)ccc2c1 ; c1ccc(-c2cc(-c3ccccc3)cc(N(c3ccccc3)c3cccc4c3ccc3cc5c(cc34)oc3ccccc35)c2)cc1]

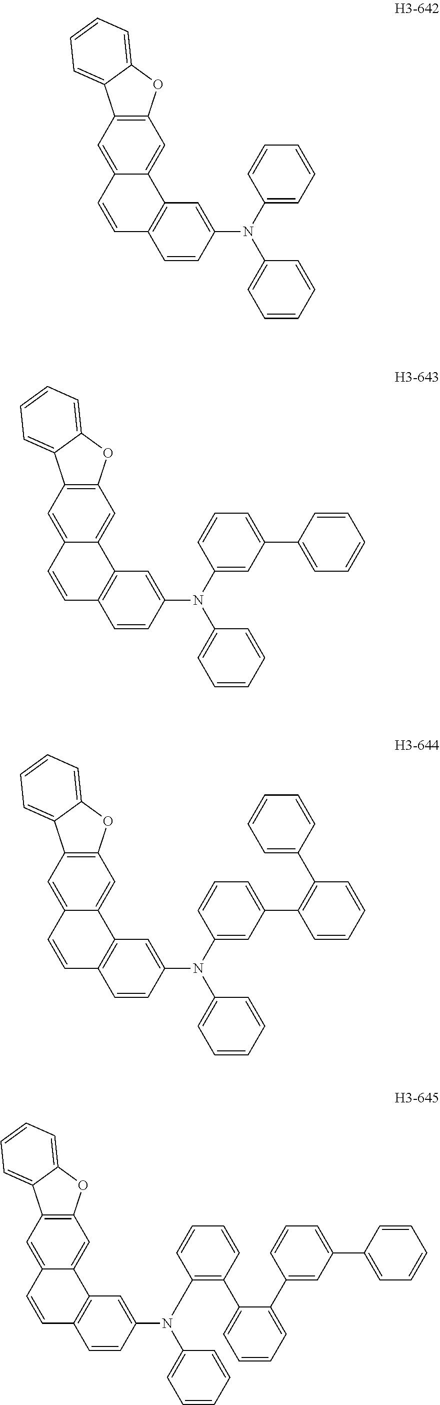 [SMILES: c1ccc(N(c2ccccc2)c2ccc3ccc4cc5c(cc4c3c2)oc2ccccc25)cc1 ; c1ccc(-c2cccc(N(c3ccccc3)c3ccc4ccc5cc6c(cc5c4c3)oc3ccccc36)c2)cc1 ; c1ccc(-c2ccccc2-c2cccc(N(c3ccccc3)c3ccc4ccc5cc6c(cc5c4c3)oc3ccccc36)c2)cc1 ; c1ccc(-c2cccc(-c3ccccc3-c3ccccc3N(c3ccccc3)c3ccc4ccc5cc6c(cc5c4c3)oc3ccccc36)c2)cc1]

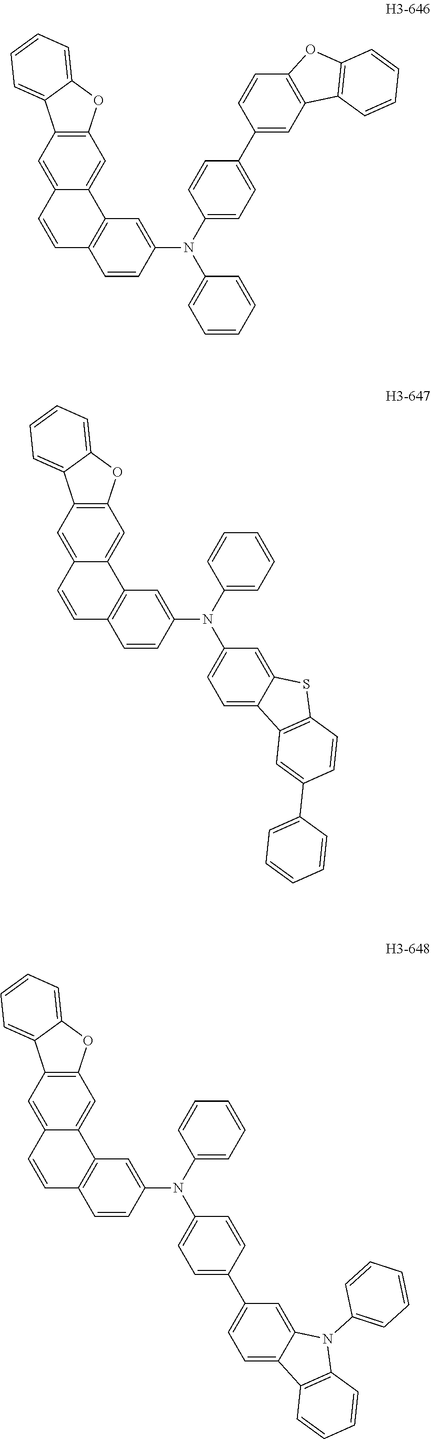 [SMILES: c1ccc(N(c2ccc(-c3ccc4oc5ccccc5c4c3)cc2)c2ccc3ccc4cc5c(cc4c3c2)oc2ccccc25)cc1 ; c1ccc(-c2ccc3sc4cc(N(c5ccccc5)c5ccc6ccc7cc8c(cc7c6c5)oc5ccccc58)ccc4c3c2)cc1 ; c1ccc(N(c2ccc(-c3ccc4c5ccccc5n(-c5ccccc5)c4c3)cc2)c2ccc3ccc4cc5c(cc4c3c2)oc2ccccc25)cc1]

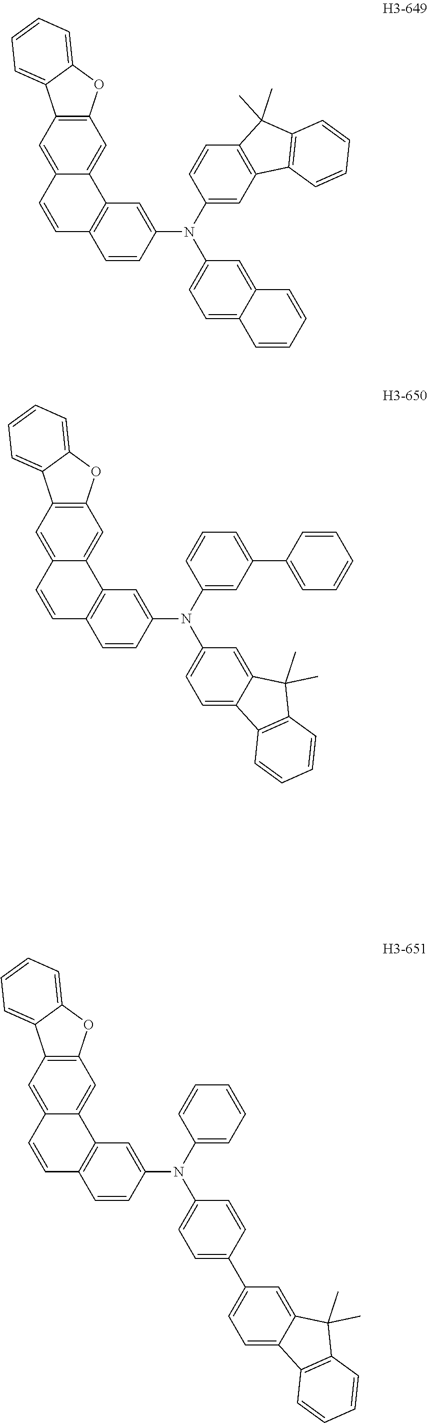 [SMILES: CC1(C)c2ccccc2-c2cc(N(c3ccc4ccccc4c3)c3ccc4ccc5cc6c(cc5c4c3)oc3ccccc36)ccc21 ; CC1(C)c2ccccc2-c2ccc(N(c3cccc(-c4ccccc4)c3)c3ccc4ccc5cc6c(cc5c4c3)oc3ccccc36)cc21 ; CC1(C)c2ccccc2-c2ccc(-c3ccc(N(c4ccccc4)c4ccc5ccc6cc7c(cc6c5c4)oc4ccccc47)cc3)cc21]

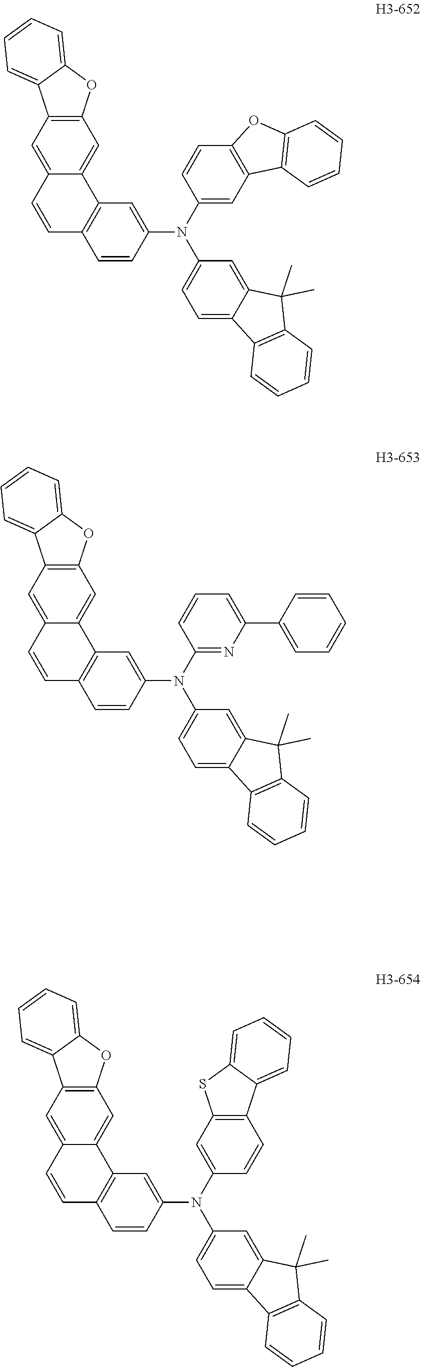 [SMILES: CC1(C)c2ccccc2-c2ccc(N(c3ccc4ccc5cc6c(cc5c4c3)oc3ccccc36)c3ccc4oc5ccccc5c4c3)cc21 ; CC1(C)c2ccccc2-c2ccc(N(c3ccc4ccc5cc6c(cc5c4c3)oc3ccccc36)c3cccc(-c4ccccc4)n3)cc21 ; CC1(C)c2ccccc2-c2ccc(N(c3ccc4c(c3)sc3ccccc34)c3ccc4ccc5cc6c(cc5c4c3)oc3ccccc36)cc21]

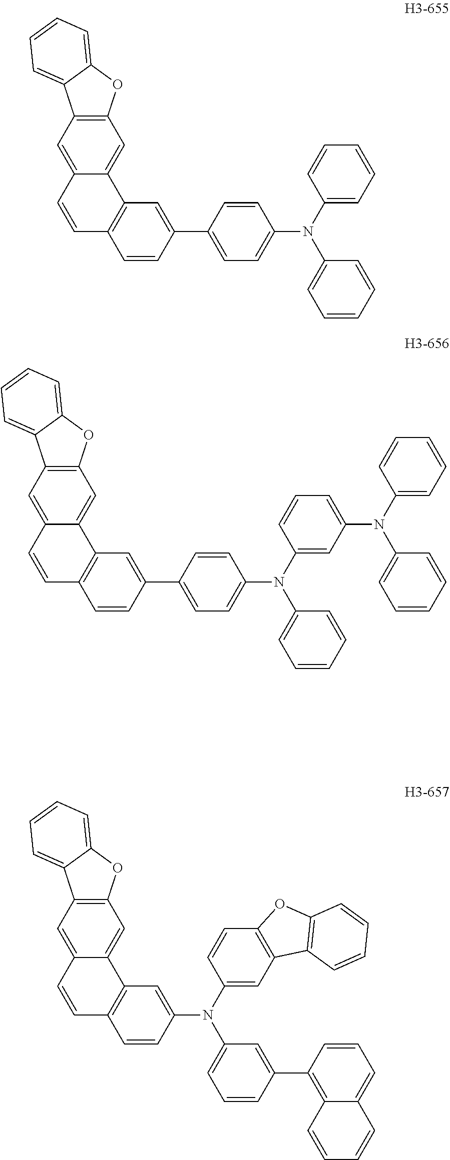 [SMILES: c1ccc(N(c2ccccc2)c2ccc(-c3ccc4ccc5cc6c(cc5c4c3)oc3ccccc36)cc2)cc1 ; c1ccc(N(c2ccccc2)c2cccc(N(c3ccccc3)c3ccc(-c4ccc5ccc6cc7c(cc6c5c4)oc4ccccc47)cc3)c2)cc1 ; c1cc(-c2cccc3ccccc23)cc(N(c2ccc3ccc4cc5c(cc4c3c2)oc2ccccc25)c2ccc3oc4ccccc4c3c2)c1]

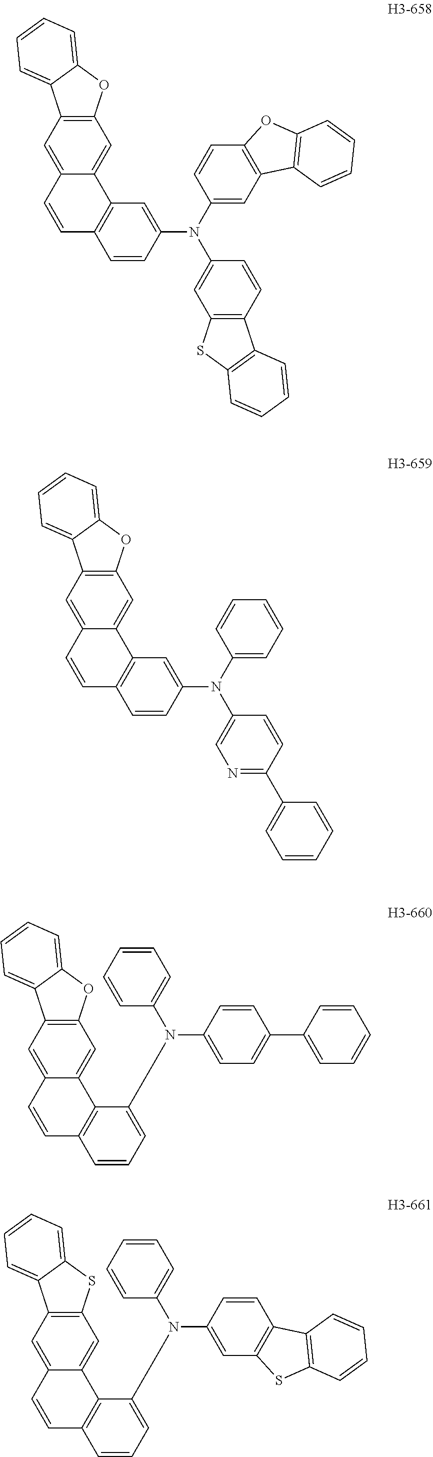 [SMILES: c1ccc2c(c1)oc1ccc(N(c3ccc4c(c3)sc3ccccc34)c3ccc4ccc5cc6c(cc5c4c3)oc3ccccc36)cc12 ; c1ccc(-c2ccc(N(c3ccccc3)c3ccc4ccc5cc6c(cc5c4c3)oc3ccccc36)cn2)cc1 ; c1ccc(-c2ccc(N(c3ccccc3)c3cccc4ccc5cc6c(cc5c34)oc3ccccc36)cc2)cc1 ; c1ccc(N(c2ccc3c(c2)sc2ccccc23)c2cccc3ccc4cc5c(cc4c23)sc2ccccc25)cc1]

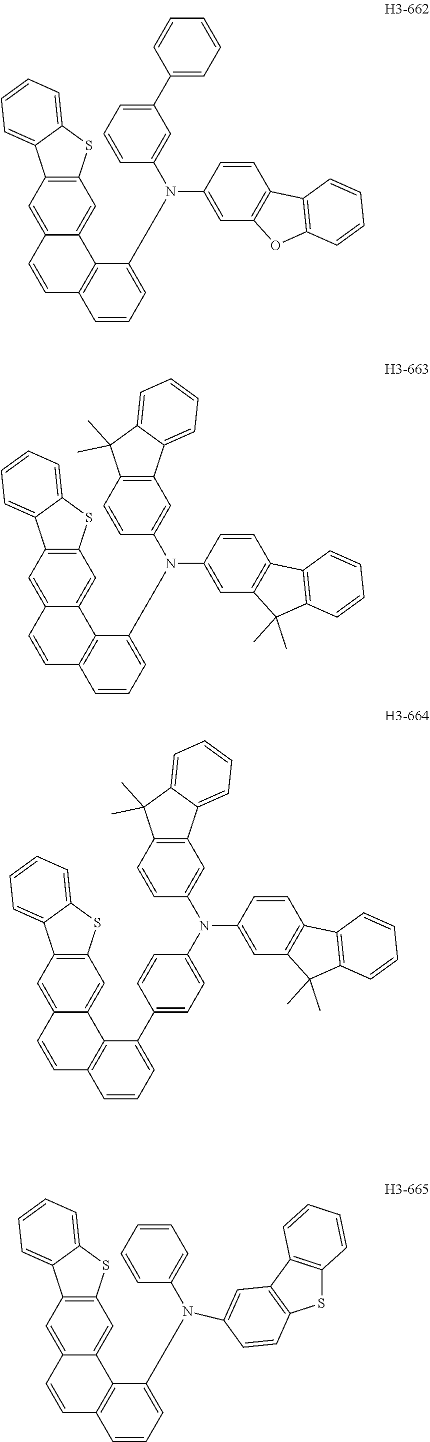 [SMILES: c1ccc(-c2cccc(N(c3ccc4c(c3)oc3ccccc34)c3cccc4ccc5cc6c(cc5c34)sc3ccccc36)c2)cc1 ; CC1(C)c2ccccc2-c2cc(N(c3ccc4c(c3)C(C)(C)c3ccccc3-4)c3cccc4ccc5cc6c(cc5c34)sc3ccccc36)ccc21 ; CC1(C)c2ccccc2-c2cc(N(c3ccc(-c4cccc5ccc6cc7c(cc6c45)sc4ccccc47)cc3)c3ccc4c(c3)C(C)(C)c3ccccc3-4)ccc21 ; c1ccc(N(c2ccc3sc4ccccc4c3c2)c2cccc3ccc4cc5c(cc4c23)sc2ccccc25)cc1]

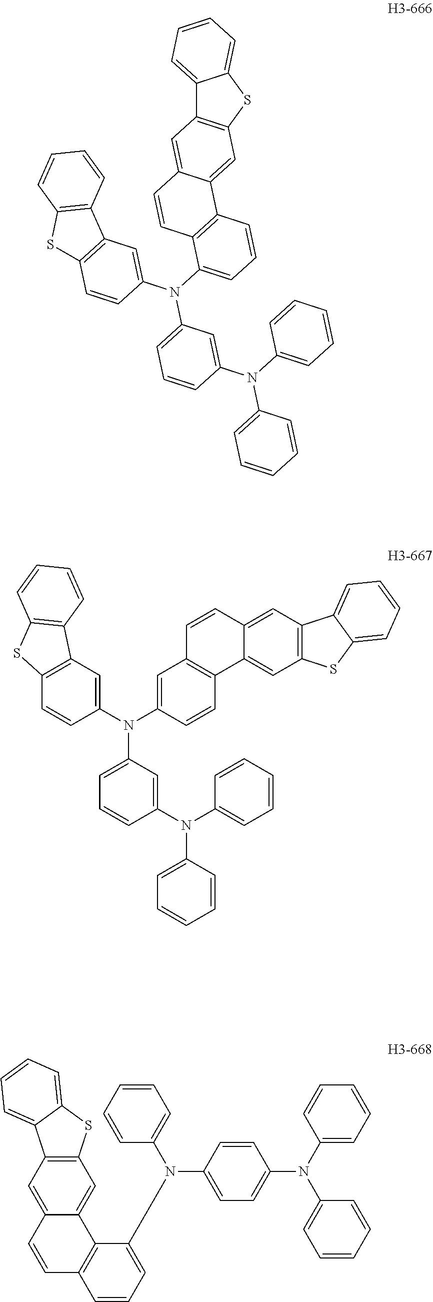 [SMILES: c1ccc(N(c2ccccc2)c2cccc(N(c3ccc4sc5ccccc5c4c3)c3cccc4c3ccc3cc5c(cc34)sc3ccccc35)c2)cc1 ; c1ccc(N(c2ccccc2)c2cccc(N(c3ccc4c(ccc5cc6c(cc54)sc4ccccc46)c3)c3ccc4sc5ccccc5c4c3)c2)cc1 ; c1ccc(N(c2ccccc2)c2ccc(N(c3ccccc3)c3cccc4ccc5cc6c(cc5c34)sc3ccccc36)cc2)cc1]

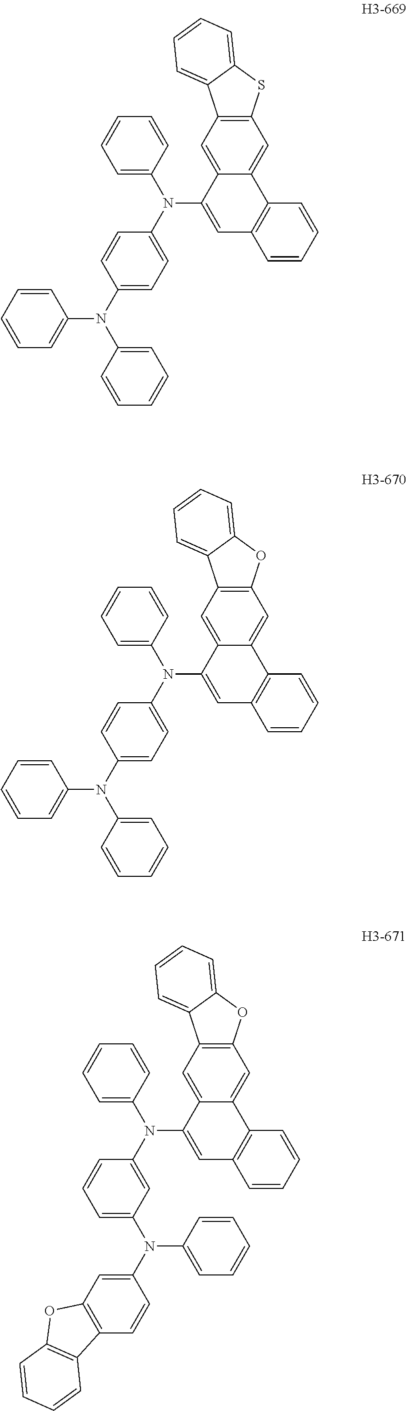 [SMILES: c1ccc(N(c2ccccc2)c2ccc(N(c3ccccc3)c3cc4ccccc4c4cc5sc6ccccc6c5cc34)cc2)cc1 ; c1ccc(N(c2ccccc2)c2ccc(N(c3ccccc3)c3cc4ccccc4c4cc5oc6ccccc6c5cc34)cc2)cc1 ; c1ccc(N(c2cccc(N(c3ccccc3)c3cc4ccccc4c4cc5oc6ccccc6c5cc34)c2)c2ccc3c(c2)oc2ccccc23)cc1]

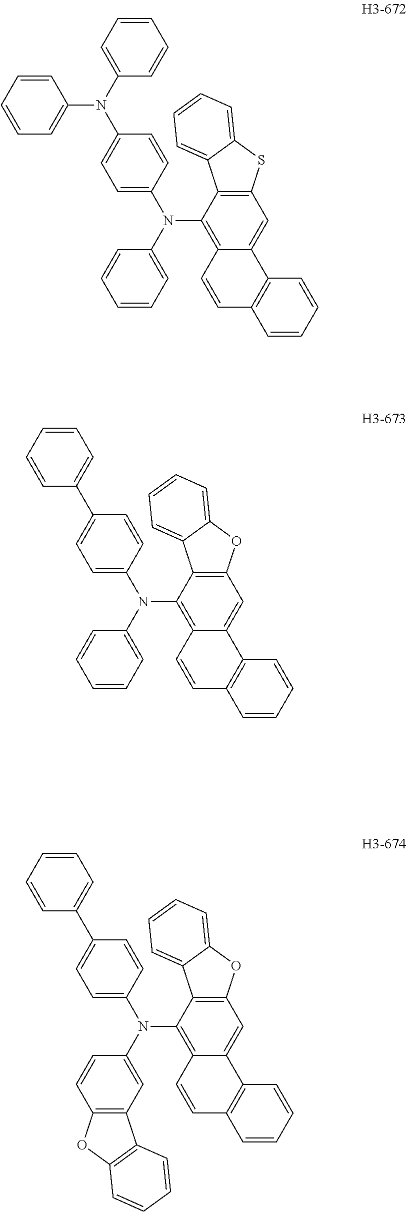 [SMILES: c1ccc(N(c2ccccc2)c2ccc(N(c3ccccc3)c3c4ccc5ccccc5c4cc4sc5ccccc5c34)cc2)cc1 ; c1ccc(-c2ccc(N(c3ccccc3)c3c4ccc5ccccc5c4cc4oc5ccccc5c34)cc2)cc1 ; c1ccc(-c2ccc(N(c3ccc4oc5ccccc5c4c3)c3c4ccc5ccccc5c4cc4oc5ccccc5c34)cc2)cc1]

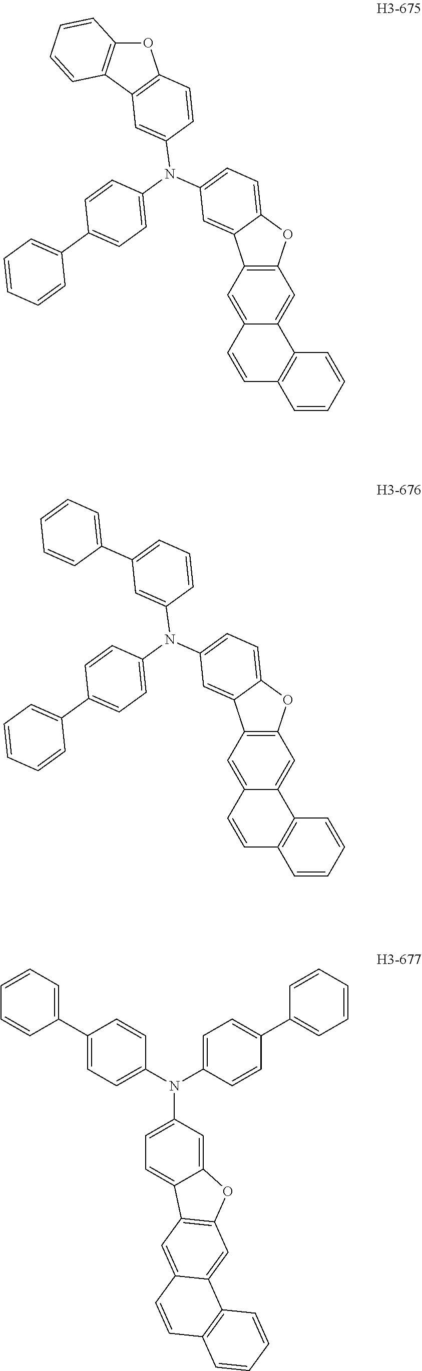 [SMILES: c1ccc(-c2ccc(N(c3ccc4oc5ccccc5c4c3)c3ccc4oc5cc6c(ccc7ccccc76)cc5c4c3)cc2)cc1 ; c1ccc(-c2ccc(N(c3cccc(-c4ccccc4)c3)c3ccc4oc5cc6c(ccc7ccccc76)cc5c4c3)cc2)cc1 ; c1ccc(-c2ccc(N(c3ccc(-c4ccccc4)cc3)c3ccc4c(c3)oc3cc5c(ccc6ccccc65)cc34)cc2)cc1]

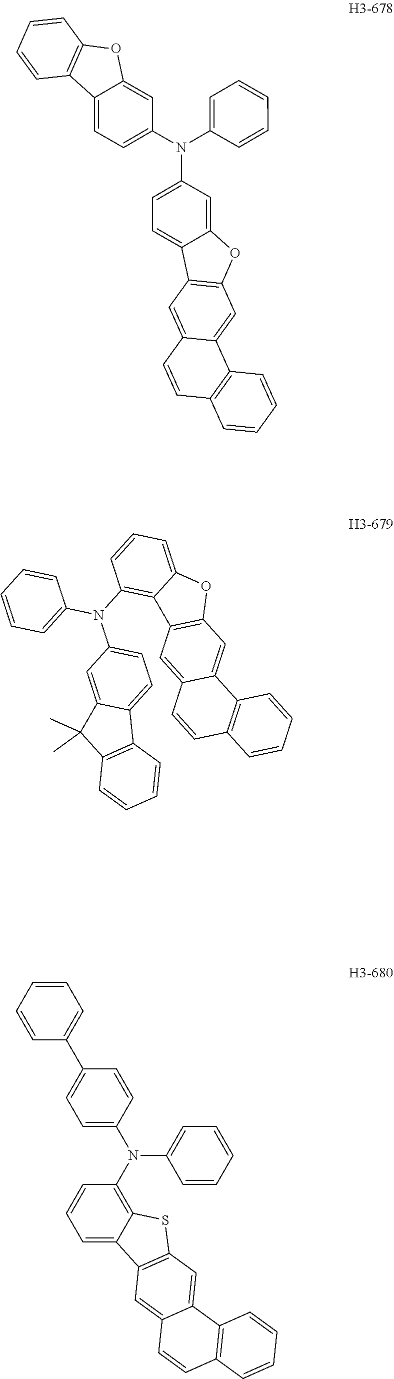 [SMILES: c1ccc(N(c2ccc3c(c2)oc2ccccc23)c2ccc3c(c2)oc2cc4c(ccc5ccccc54)cc23)cc1 ; CC1(C)c2ccccc2-c2ccc(N(c3ccccc3)c3cccc4oc5cc6c(ccc7ccccc76)cc5c34)cc21 ; c1ccc(-c2ccc(N(c3ccccc3)c3cccc4c3sc3cc5c(ccc6ccccc65)cc34)cc2)cc1]

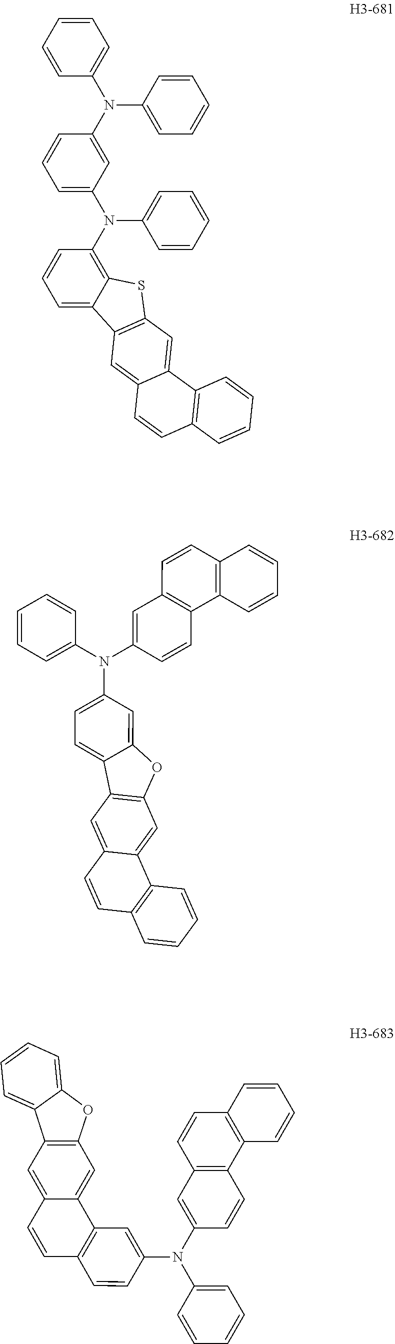 [SMILES: c1ccc(N(c2ccccc2)c2cccc(N(c3ccccc3)c3cccc4c3sc3cc5c(ccc6ccccc65)cc34)c2)cc1 ; c1ccc(N(c2ccc3c(ccc4ccccc43)c2)c2ccc3c(c2)oc2cc4c(ccc5ccccc54)cc23)cc1 ; c1ccc(N(c2ccc3c(ccc4ccccc43)c2)c2ccc3ccc4cc5c(cc4c3c2)oc2ccccc25)cc1]

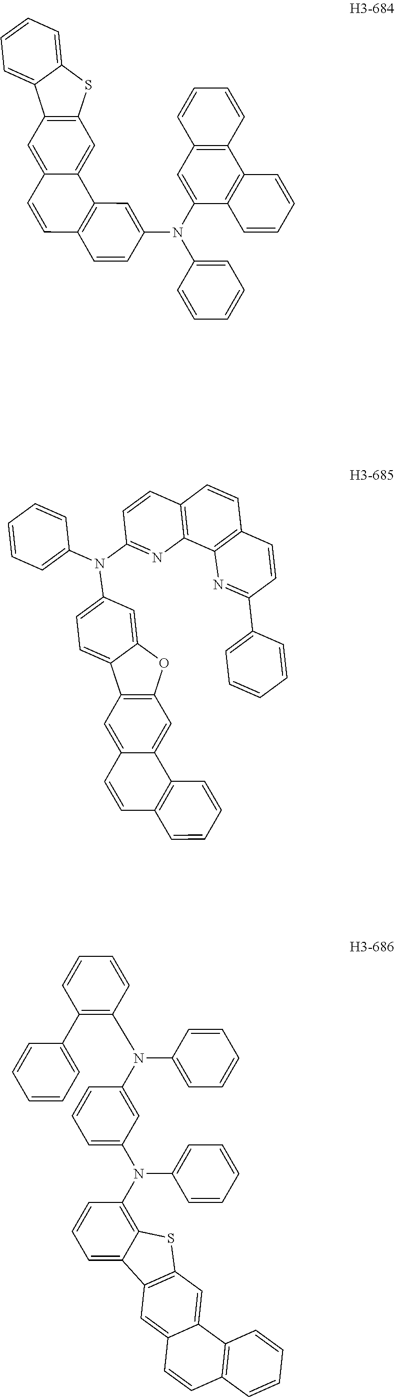 [SMILES: c1ccc(N(c2ccc3ccc4cc5c(cc4c3c2)sc2ccccc25)c2cc3ccccc3c3ccccc23)cc1 ; c1ccc(-c2ccc3ccc4ccc(N(c5ccccc5)c5ccc6c(c5)oc5cc7c(ccc8ccccc87)cc56)nc4c3n2)cc1 ; c1ccc(-c2ccccc2N(c2ccccc2)c2cccc(N(c3ccccc3)c3cccc4c3sc3cc5c(ccc6ccccc65)cc34)c2)cc1]

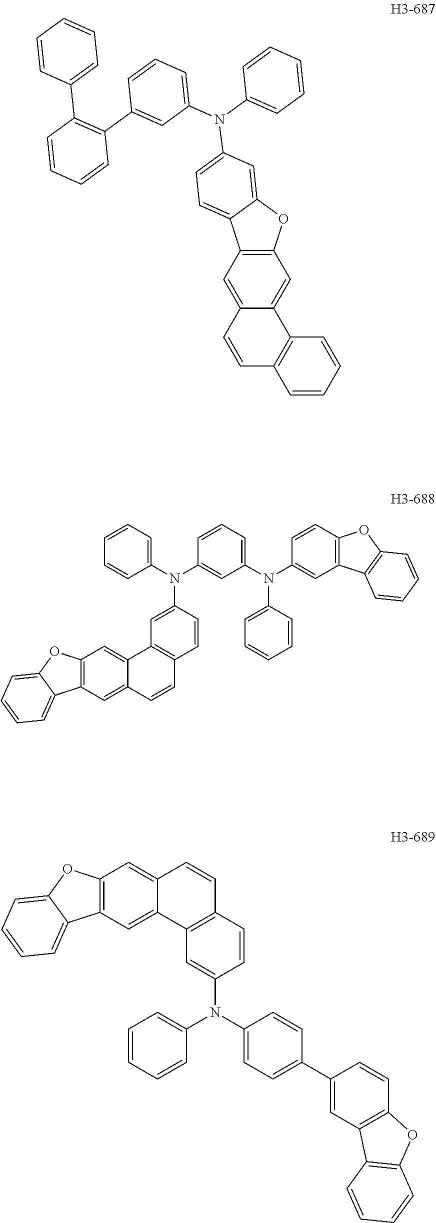 [SMILES: c1ccc(-c2ccccc2-c2cccc(N(c3ccccc3)c3ccc4c(c3)oc3cc5c(ccc6ccccc65)cc34)c2)cc1 ; c1ccc(N(c2cccc(N(c3ccccc3)c3ccc4oc5ccccc5c4c3)c2)c2ccc3ccc4cc5c(cc4c3c2)oc2ccccc25)cc1 ; c1ccc(N(c2ccc(-c3ccc4oc5ccccc5c4c3)cc2)c2ccc3ccc4cc5oc6ccccc6c5cc4c3c2)cc1]

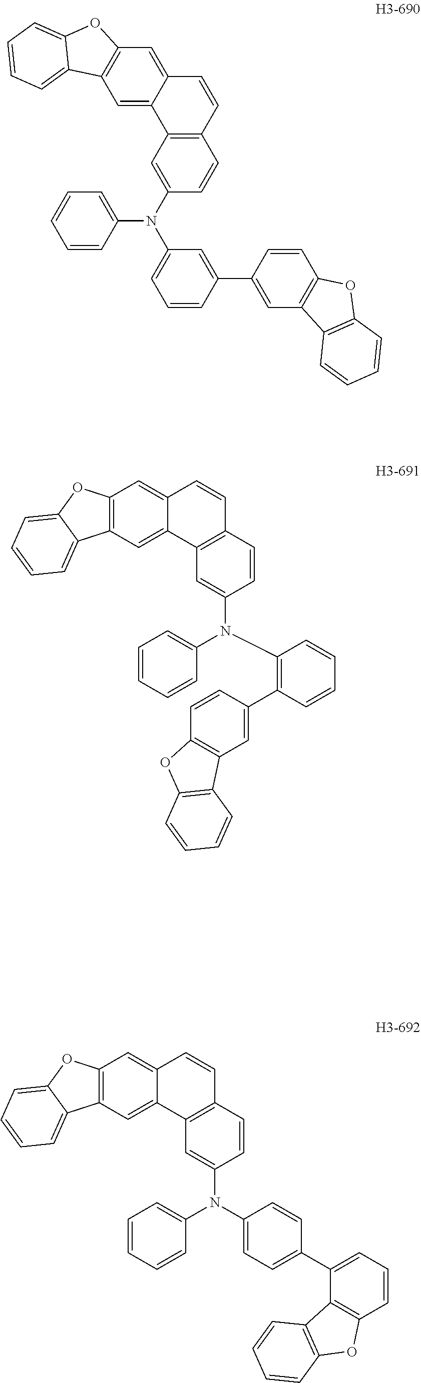 [SMILES: c1ccc(N(c2cccc(-c3ccc4oc5ccccc5c4c3)c2)c2ccc3ccc4cc5oc6ccccc6c5cc4c3c2)cc1 ; c1ccc(N(c2ccc3ccc4cc5oc6ccccc6c5cc4c3c2)c2ccccc2-c2ccc3oc4ccccc4c3c2)cc1 ; c1ccc(N(c2ccc(-c3cccc4oc5ccccc5c34)cc2)c2ccc3ccc4cc5oc6ccccc6c5cc4c3c2)cc1]

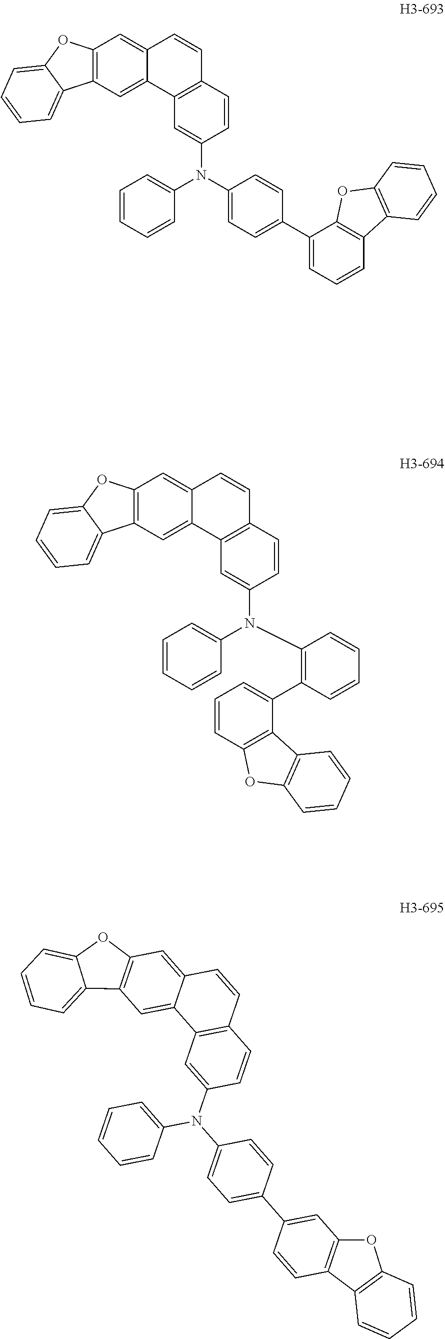 [SMILES: c1ccc(N(c2ccc(-c3cccc4c3oc3ccccc34)cc2)c2ccc3ccc4cc5oc6ccccc6c5cc4c3c2)cc1 ; c1ccc(N(c2ccc3ccc4cc5oc6ccccc6c5cc4c3c2)c2ccccc2-c2cccc3oc4ccccc4c23)cc1 ; c1ccc(N(c2ccc(-c3ccc4c(c3)oc3ccccc34)cc2)c2ccc3ccc4cc5oc6ccccc6c5cc4c3c2)cc1]

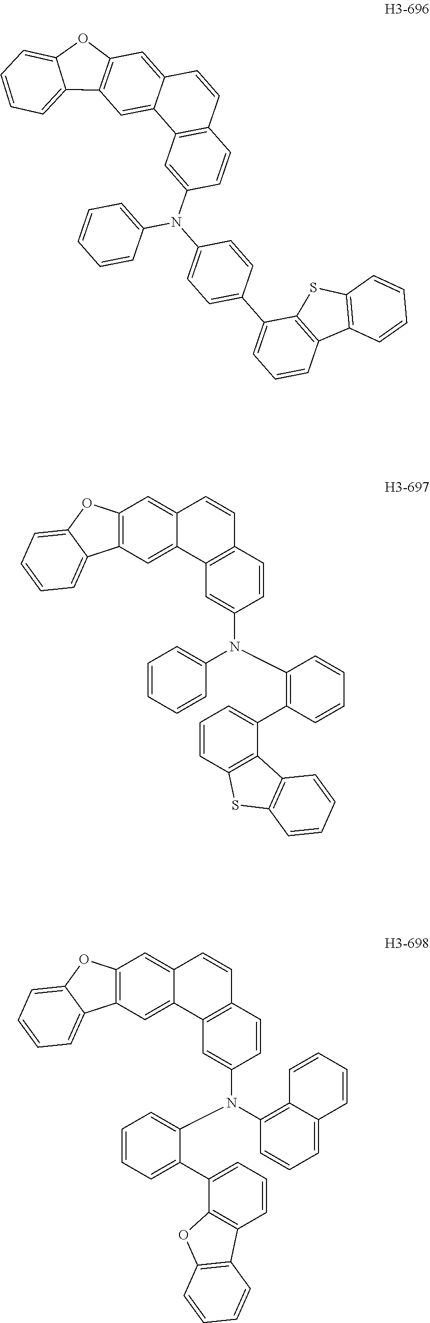 [SMILES: c1ccc(N(c2ccc(-c3cccc4c3sc3ccccc34)cc2)c2ccc3ccc4cc5oc6ccccc6c5cc4c3c2)cc1 ; c1ccc(N(c2ccc3ccc4cc5oc6ccccc6c5cc4c3c2)c2ccccc2-c2cccc3sc4ccccc4c23)cc1 ; c1ccc(N(c2ccc3ccc4cc5oc6ccccc6c5cc4c3c2)c2cccc3ccccc23)c(-c2cccc3c2oc2ccccc23)c1]

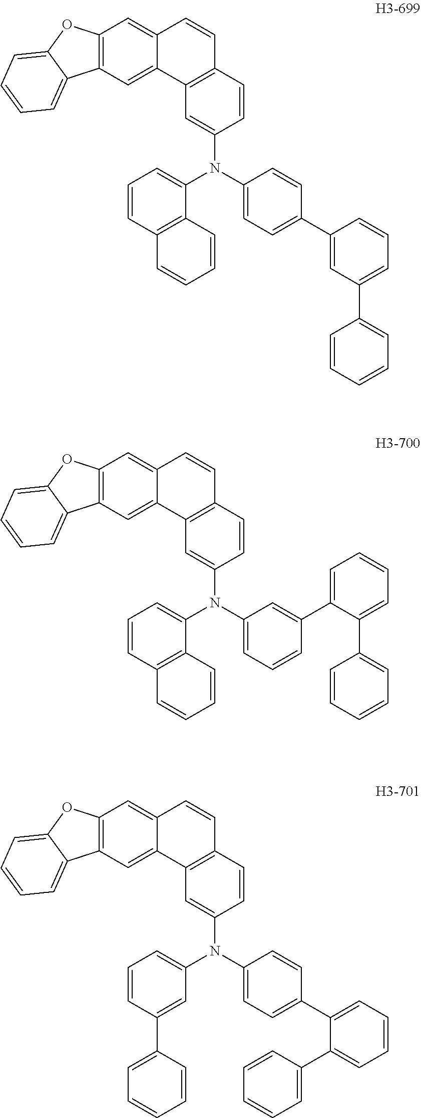 [SMILES: c1ccc(-c2cccc(-c3ccc(N(c4ccc5ccc6cc7oc8ccccc8c7cc6c5c4)c4cccc5ccccc45)cc3)c2)cc1 ; c1ccc(-c2ccccc2-c2cccc(N(c3ccc4ccc5cc6oc7ccccc7c6cc5c4c3)c3cccc4ccccc34)c2)cc1 ; c1ccc(-c2cccc(N(c3ccc(-c4ccccc4-c4ccccc4)cc3)c3ccc4ccc5cc6oc7ccccc7c6cc5c4c3)c2)cc1]

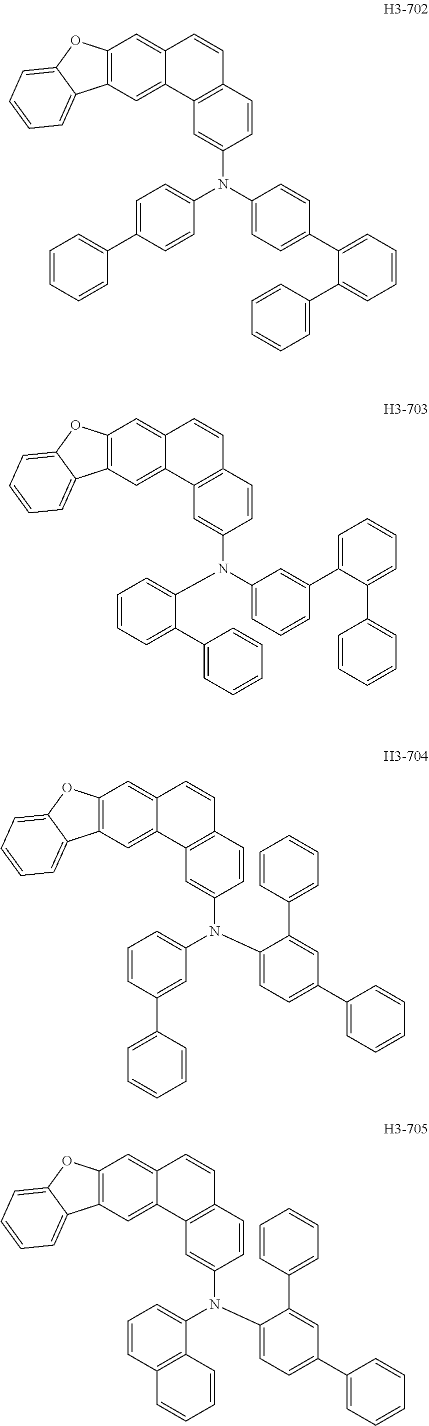 [SMILES: c1ccc(-c2ccc(N(c3ccc(-c4ccccc4-c4ccccc4)cc3)c3ccc4ccc5cc6oc7ccccc7c6cc5c4c3)cc2)cc1 ; c1ccc(-c2ccccc2-c2cccc(N(c3ccc4ccc5cc6oc7ccccc7c6cc5c4c3)c3ccccc3-c3ccccc3)c2)cc1 ; c1ccc(-c2cccc(N(c3ccc4ccc5cc6oc7ccccc7c6cc5c4c3)c3ccc(-c4ccccc4)cc3-c3ccccc3)c2)cc1 ; c1ccc(-c2ccc(N(c3ccc4ccc5cc6oc7ccccc7c6cc5c4c3)c3cccc4ccccc34)c(-c3ccccc3)c2)cc1]

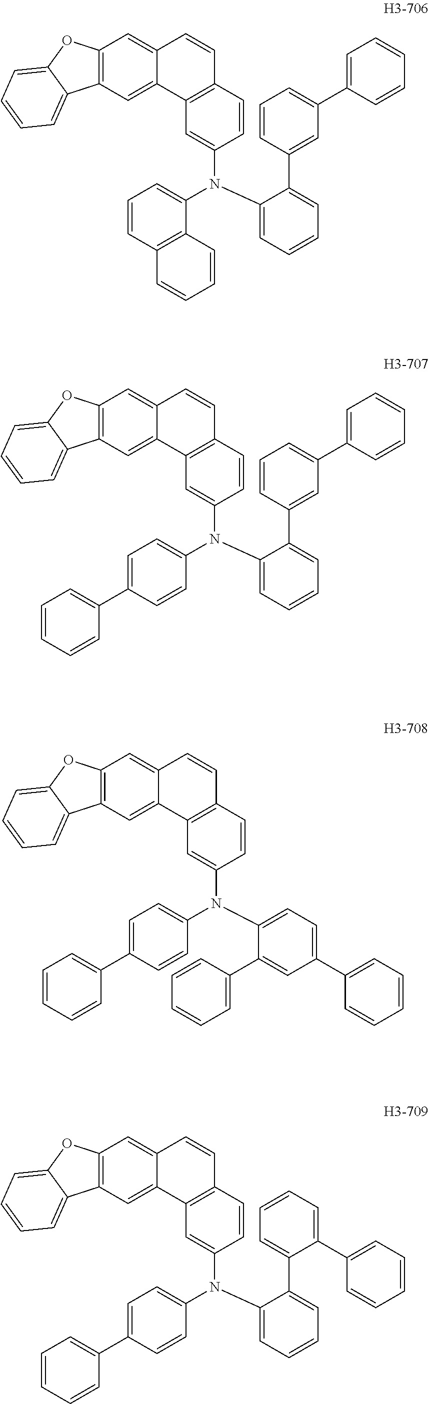 [SMILES: c1ccc(-c2cccc(-c3ccccc3N(c3ccc4ccc5cc6oc7ccccc7c6cc5c4c3)c3cccc4ccccc34)c2)cc1 ; c1ccc(-c2ccc(N(c3ccc4ccc5cc6oc7ccccc7c6cc5c4c3)c3ccccc3-c3cccc(-c4ccccc4)c3)cc2)cc1 ; c1ccc(-c2ccc(N(c3ccc4ccc5cc6oc7ccccc7c6cc5c4c3)c3ccc(-c4ccccc4)cc3-c3ccccc3)cc2)cc1 ; c1ccc(-c2ccc(N(c3ccc4ccc5cc6oc7ccccc7c6cc5c4c3)c3ccccc3-c3ccccc3-c3ccccc3)cc2)cc1]

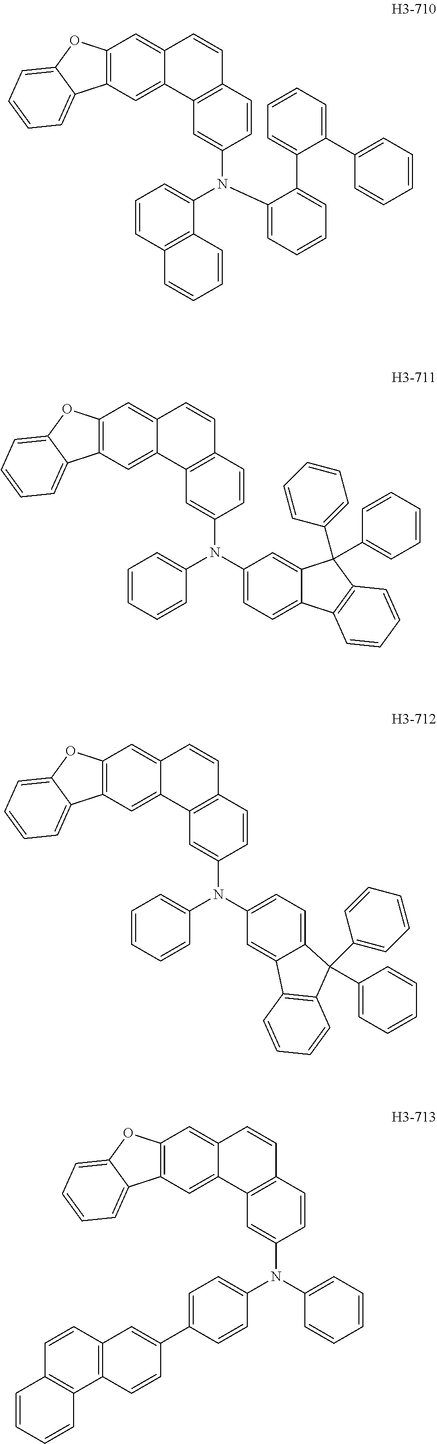 [SMILES: c1ccc(-c2ccccc2-c2ccccc2N(c2ccc3ccc4cc5oc6ccccc6c5cc4c3c2)c2cccc3ccccc23)cc1 ; c1ccc(N(c2ccc3c(c2)C(c2ccccc2)(c2ccccc2)c2ccccc2-3)c2ccc3ccc4cc5oc6ccccc6c5cc4c3c2)cc1 ; c1ccc(N(c2ccc3c(c2)-c2ccccc2C3(c2ccccc2)c2ccccc2)c2ccc3ccc4cc5oc6ccccc6c5cc4c3c2)cc1 ; c1ccc(N(c2ccc(-c3ccc4c(ccc5ccccc54)c3)cc2)c2ccc3ccc4cc5oc6ccccc6c5cc4c3c2)cc1]

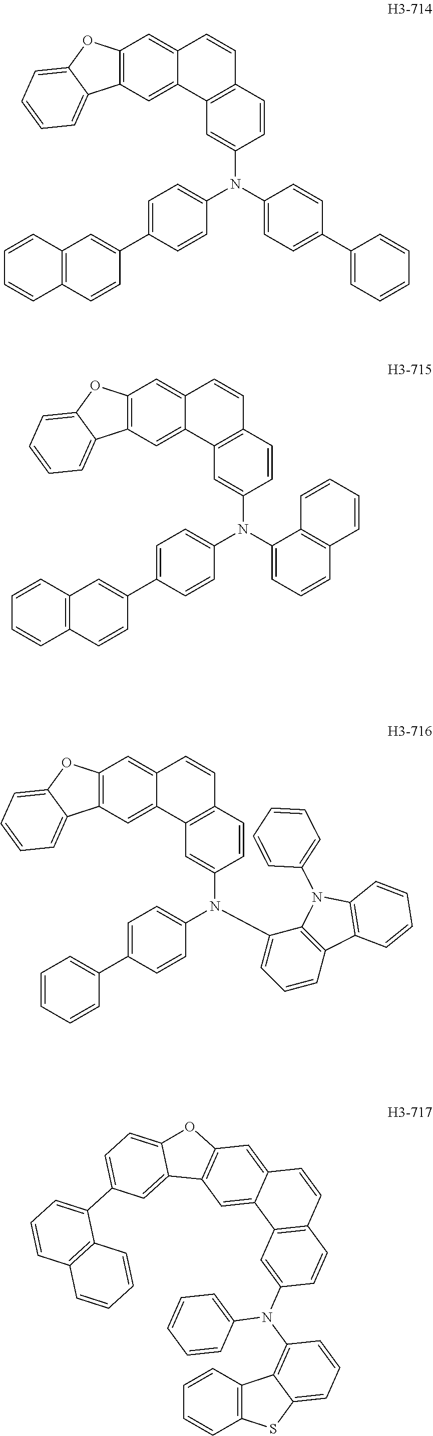 [SMILES: c1ccc(-c2ccc(N(c3ccc(-c4ccc5ccccc5c4)cc3)c3ccc4ccc5cc6oc7ccccc7c6cc5c4c3)cc2)cc1 ; c1ccc2cc(-c3ccc(N(c4ccc5ccc6cc7oc8ccccc8c7cc6c5c4)c4cccc5ccccc45)cc3)ccc2c1 ; c1ccc(-c2ccc(N(c3ccc4ccc5cc6oc7ccccc7c6cc5c4c3)c3cccc4c5ccccc5n(-c5ccccc5)c34)cc2)cc1 ; c1ccc(N(c2ccc3ccc4cc5oc6ccc(-c7cccc8ccccc78)cc6c5cc4c3c2)c2cccc3sc4ccccc4c23)cc1]

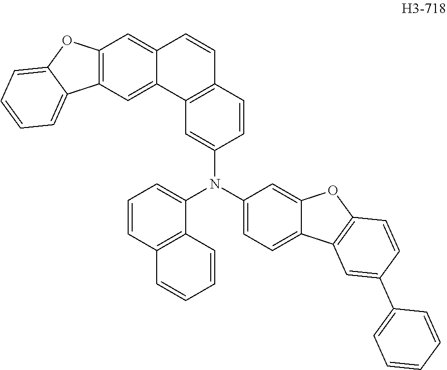 [SMILES: c1ccc(-c2ccc3oc4cc(N(c5ccc6ccc7cc8oc9ccccc9c8cc7c6c5)c5cccc6ccccc56)ccc4c3c2)cc1]

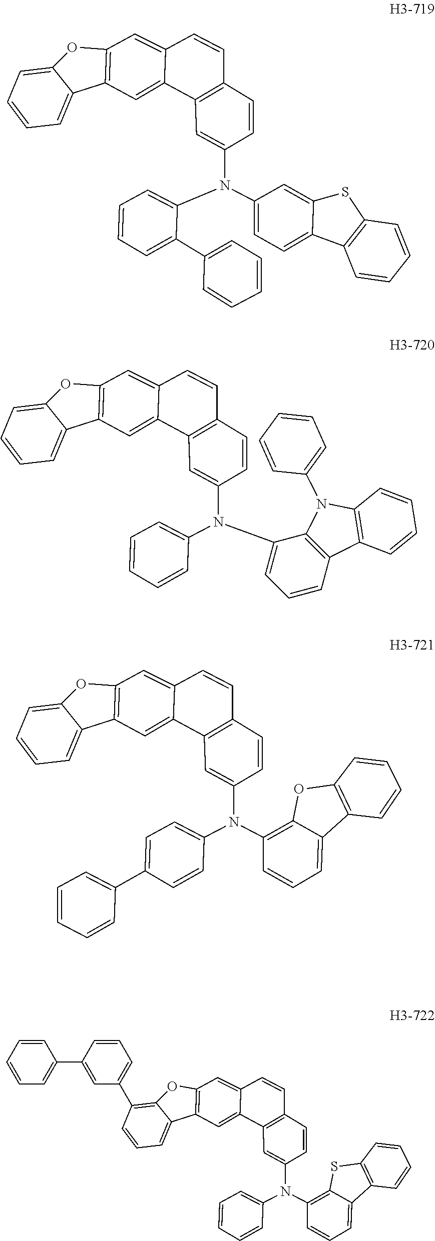 [SMILES: c1ccc(-c2ccccc2N(c2ccc3c(c2)sc2ccccc23)c2ccc3ccc4cc5oc6ccccc6c5cc4c3c2)cc1 ; c1ccc(N(c2ccc3ccc4cc5oc6ccccc6c5cc4c3c2)c2cccc3c4ccccc4n(-c4ccccc4)c23)cc1 ; c1ccc(-c2ccc(N(c3ccc4ccc5cc6oc7ccccc7c6cc5c4c3)c3cccc4c3oc3ccccc34)cc2)cc1 ; c1ccc(-c2cccc(-c3cccc4c3oc3cc5ccc6ccc(N(c7ccccc7)c7cccc8c7sc7ccccc78)cc6c5cc34)c2)cc1]

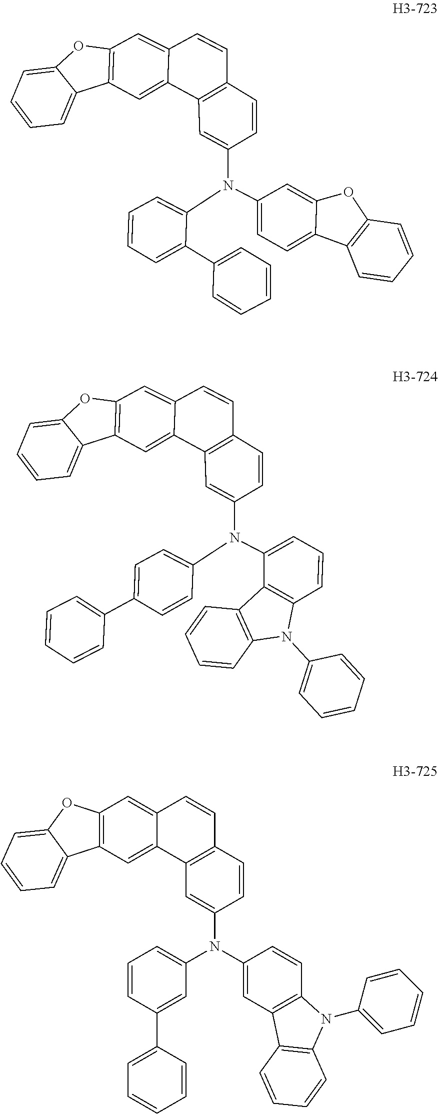 [SMILES: c1ccc(-c2ccccc2N(c2ccc3c(c2)oc2ccccc23)c2ccc3ccc4cc5oc6ccccc6c5cc4c3c2)cc1 ; c1ccc(-c2ccc(N(c3ccc4ccc5cc6oc7ccccc7c6cc5c4c3)c3cccc4c3c3ccccc3n4-c3ccccc3)cc2)cc1 ; c1ccc(-c2cccc(N(c3ccc4ccc5cc6oc7ccccc7c6cc5c4c3)c3ccc4c(c3)c3ccccc3n4-c3ccccc3)c2)cc1]

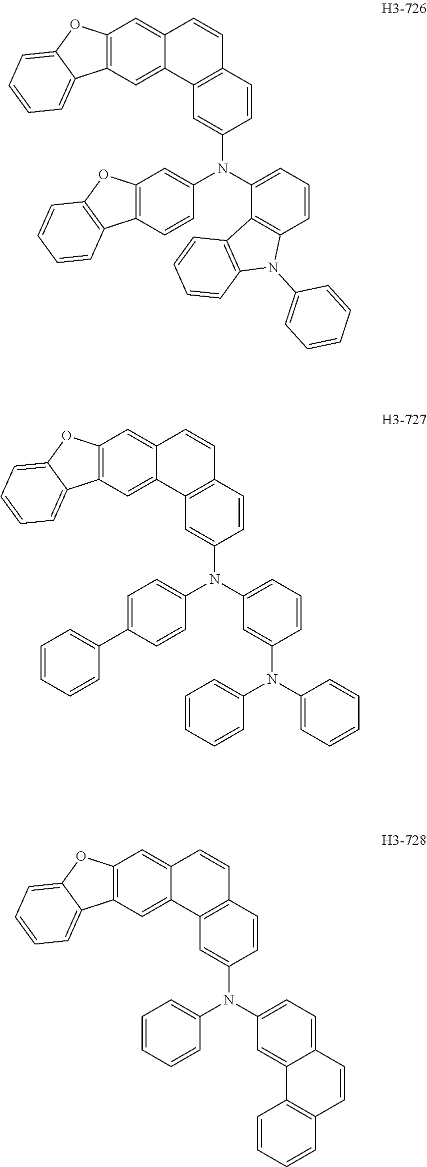 [SMILES: c1ccc(-n2c3ccccc3c3c(N(c4ccc5c(c4)oc4ccccc45)c4ccc5ccc6cc7oc8ccccc8c7cc6c5c4)cccc32)cc1 ; c1ccc(-c2ccc(N(c3cccc(N(c4ccccc4)c4ccccc4)c3)c3ccc4ccc5cc6oc7ccccc7c6cc5c4c3)cc2)cc1 ; c1ccc(N(c2ccc3ccc4ccccc4c3c2)c2ccc3ccc4cc5oc6ccccc6c5cc4c3c2)cc1]

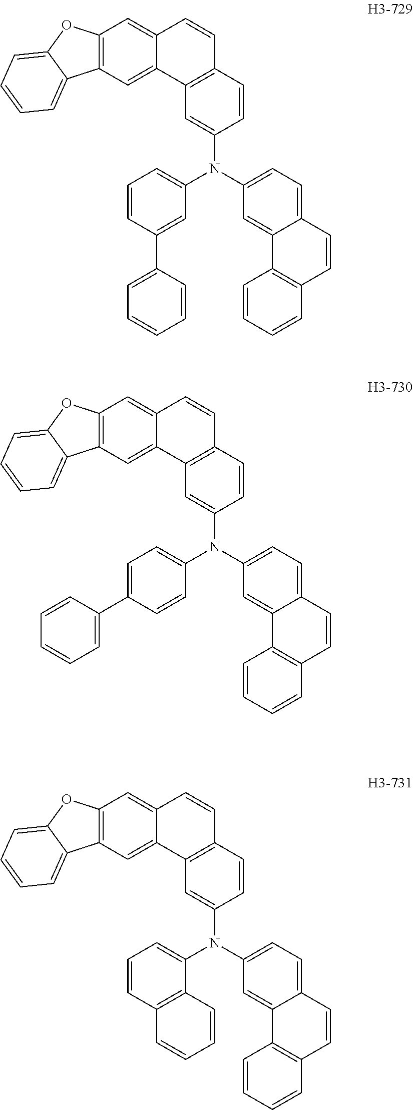 [SMILES: c1ccc(-c2cccc(N(c3ccc4ccc5ccccc5c4c3)c3ccc4ccc5cc6oc7ccccc7c6cc5c4c3)c2)cc1 ; c1ccc(-c2ccc(N(c3ccc4ccc5ccccc5c4c3)c3ccc4ccc5cc6oc7ccccc7c6cc5c4c3)cc2)cc1 ; c1ccc2c(N(c3ccc4ccc5ccccc5c4c3)c3ccc4ccc5cc6oc7ccccc7c6cc5c4c3)cccc2c1]

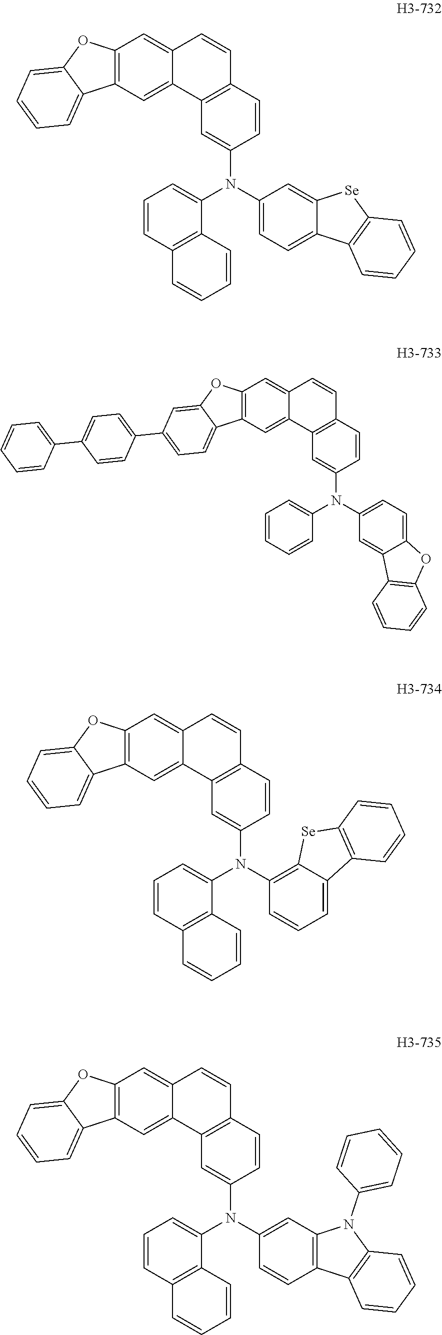 [SMILES: c1ccc2c(N(c3ccc4c(c3)[se]c3ccccc34)c3ccc4ccc5cc6oc7ccccc7c6cc5c4c3)cccc2c1 ; c1ccc(-c2ccc(-c3ccc4c(c3)oc3cc5ccc6ccc(N(c7ccccc7)c7ccc8oc9ccccc9c8c7)cc6c5cc34)cc2)cc1 ; c1ccc2c(N(c3ccc4ccc5cc6oc7ccccc7c6cc5c4c3)c3cccc4c3[se]c3ccccc34)cccc2c1 ; c1ccc(-n2c3ccccc3c3ccc(N(c4ccc5ccc6cc7oc8ccccc8c7cc6c5c4)c4cccc5ccccc45)cc32)cc1]

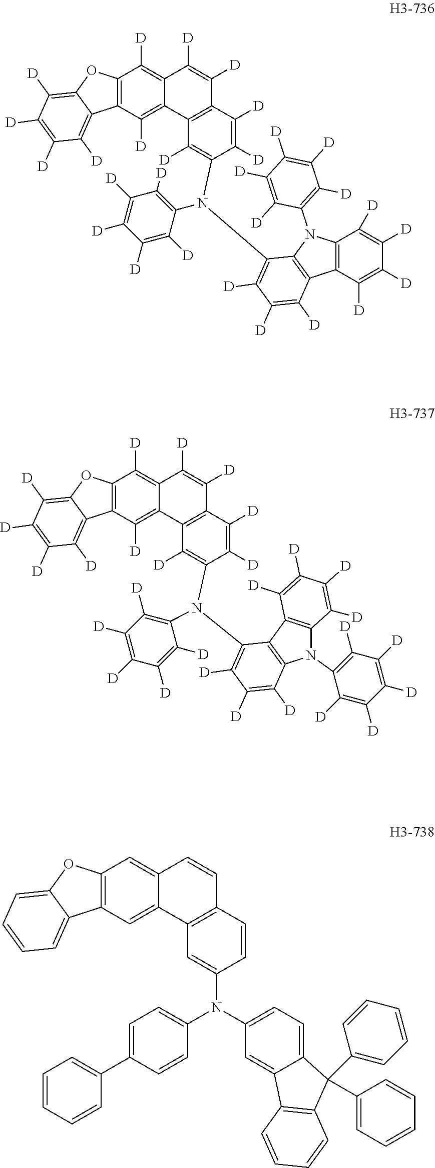

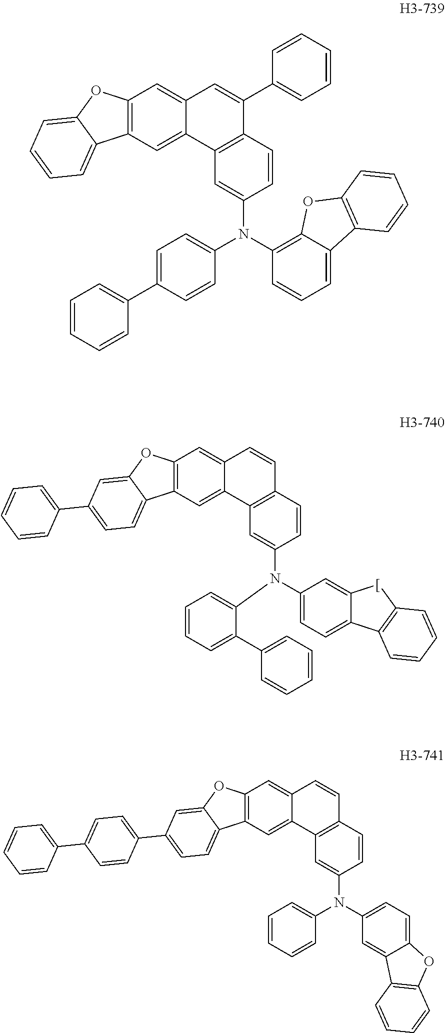

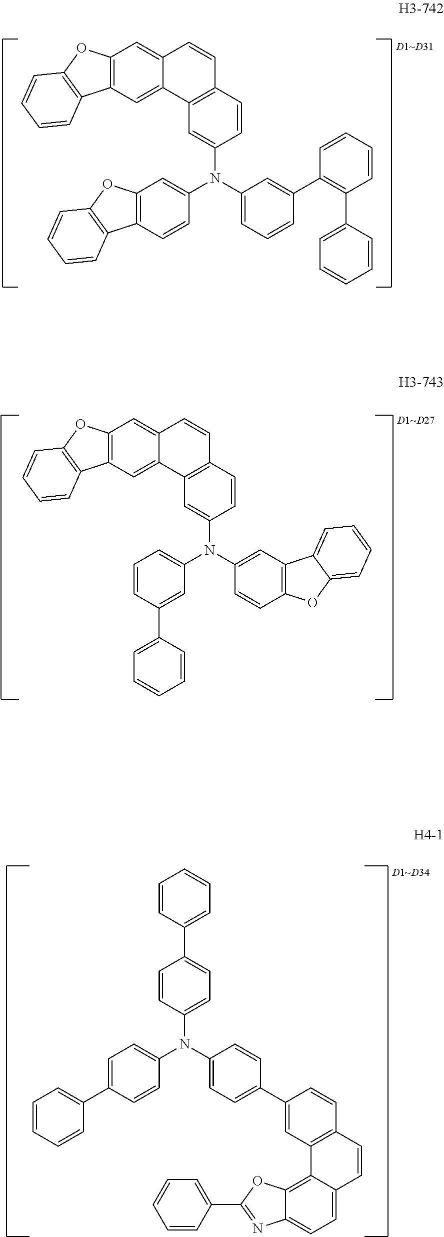

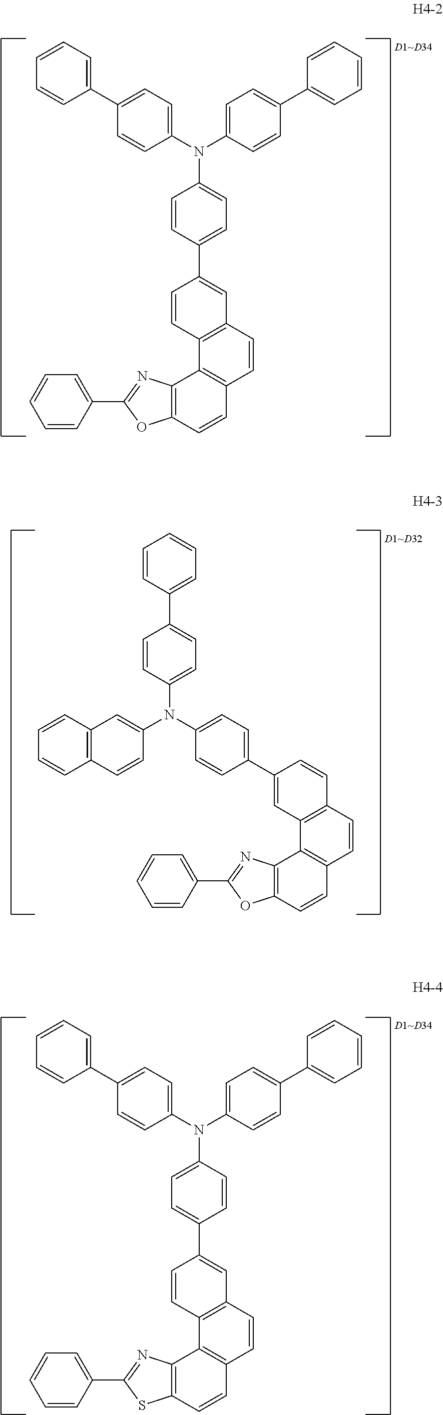

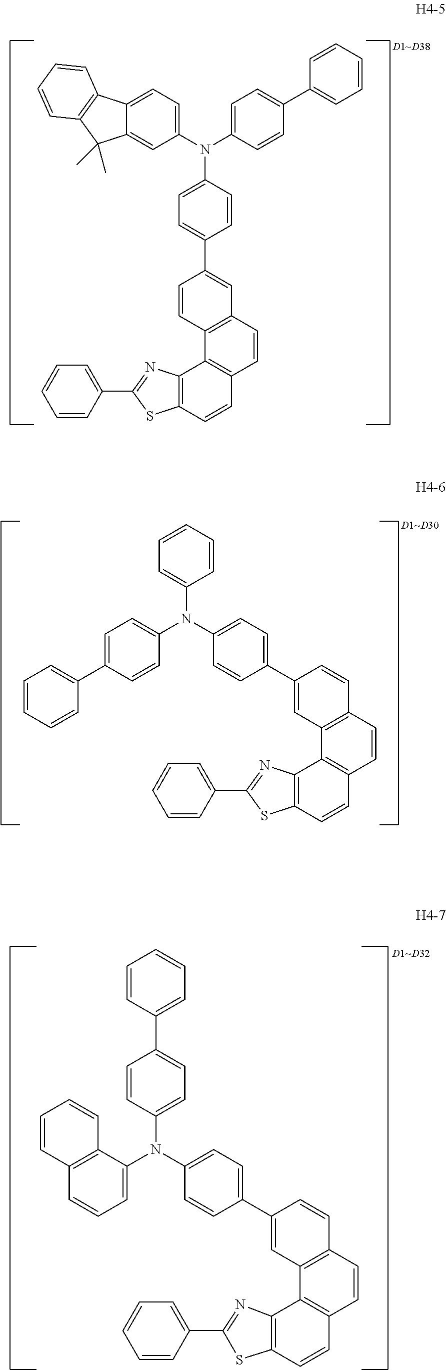

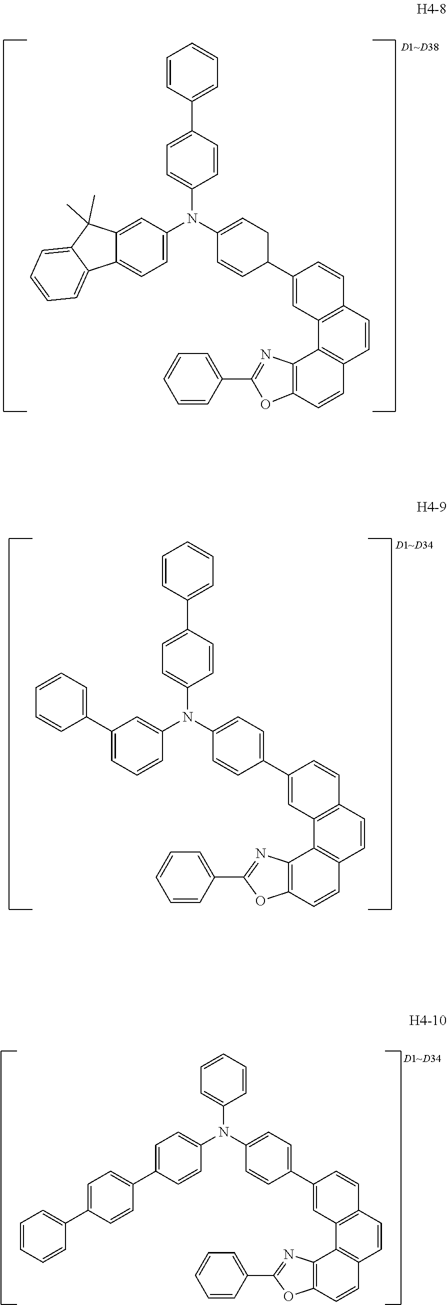

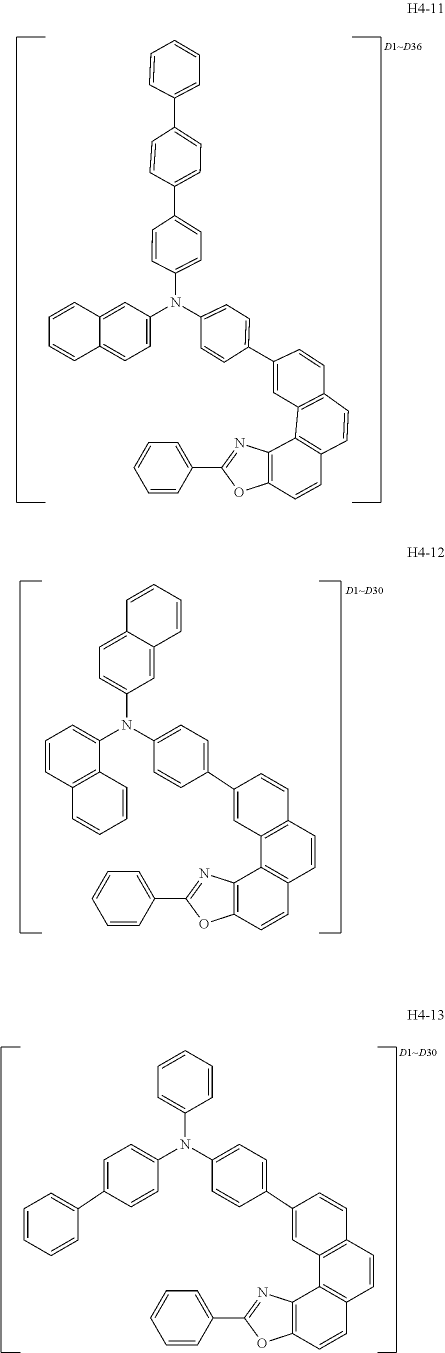

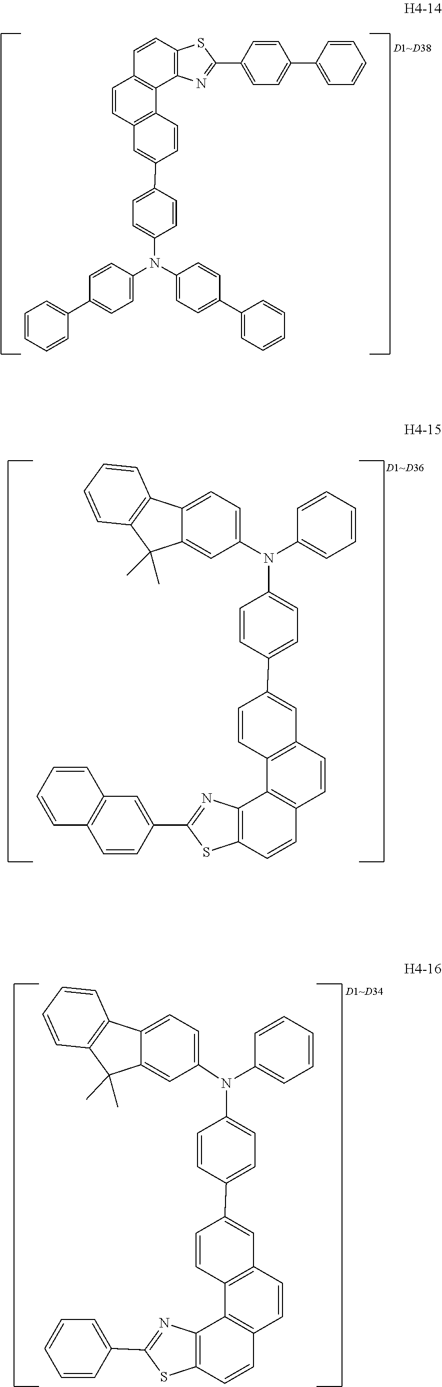

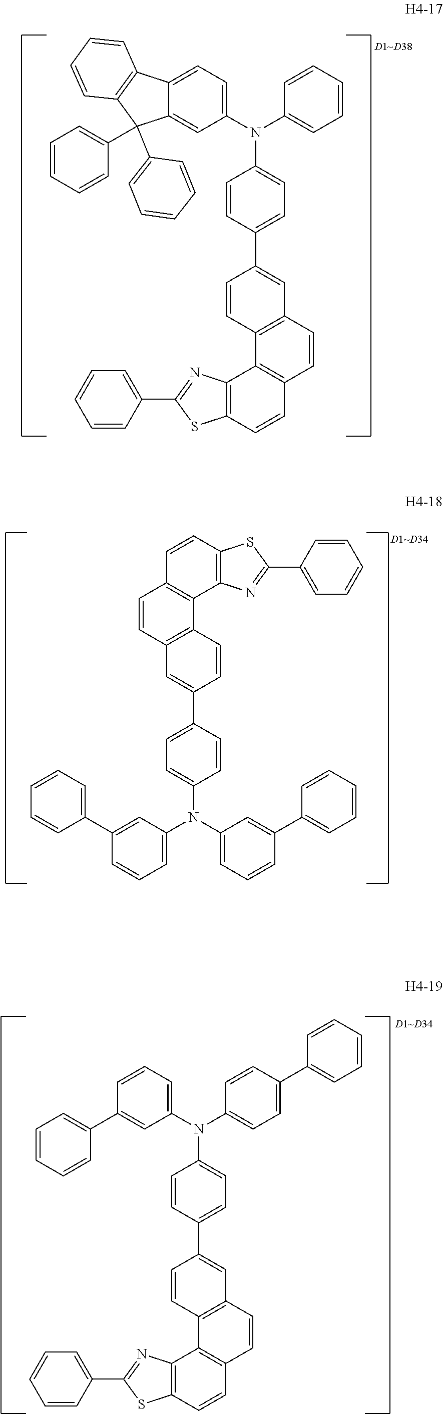

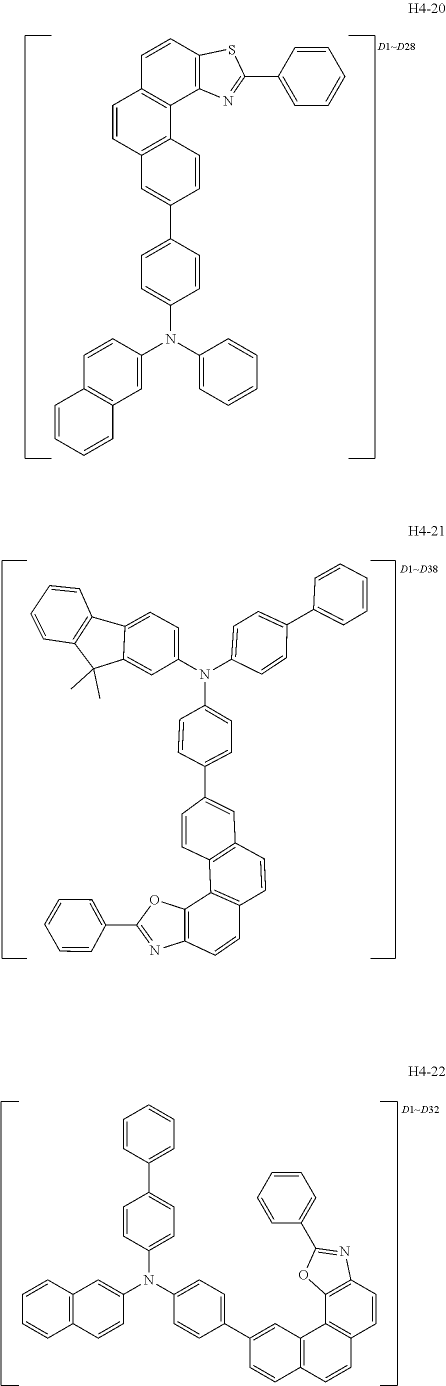

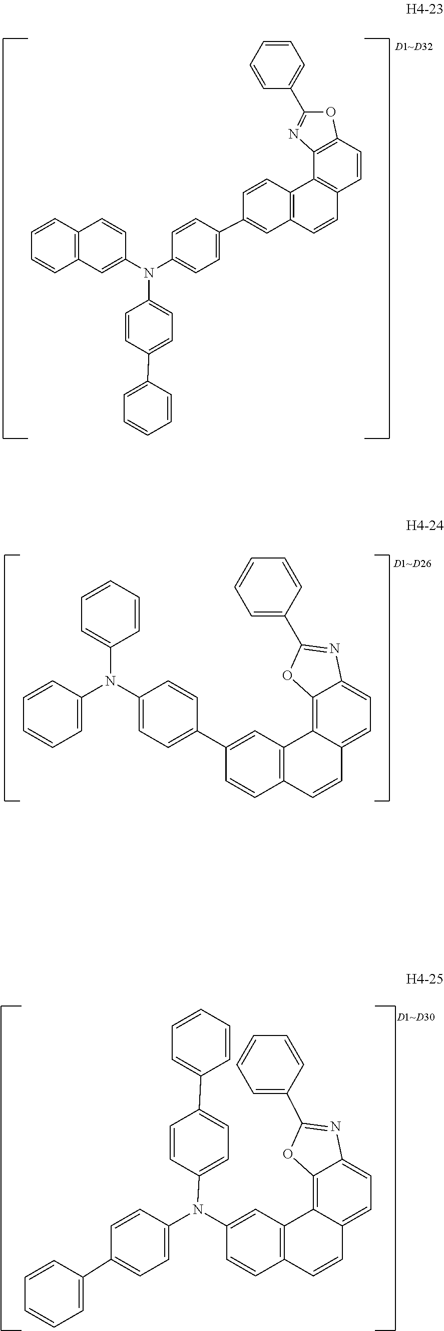

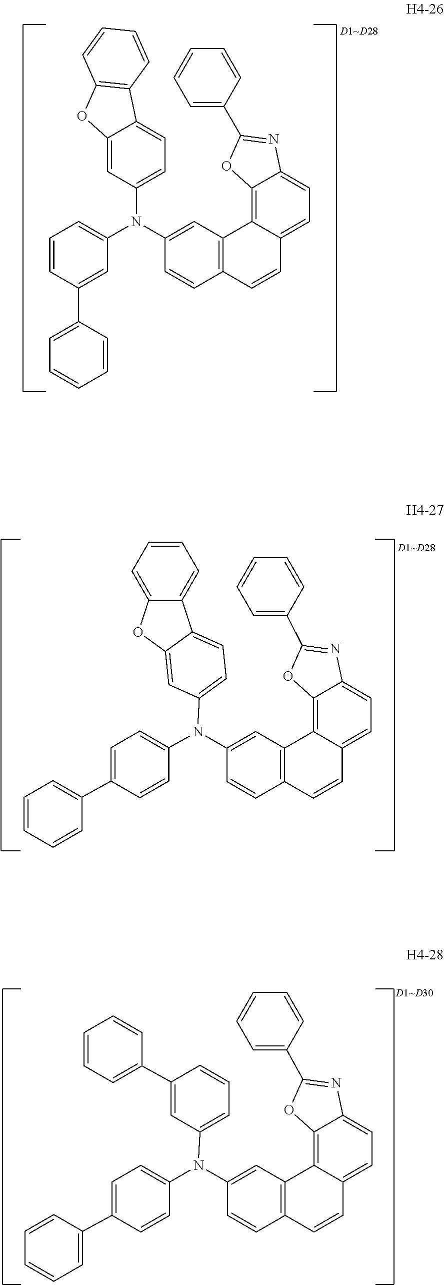

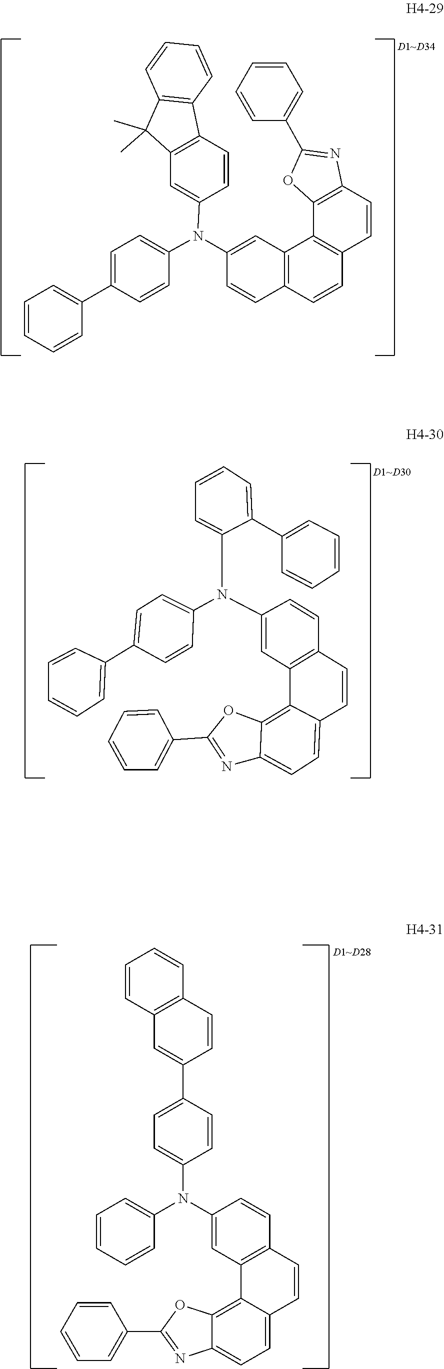

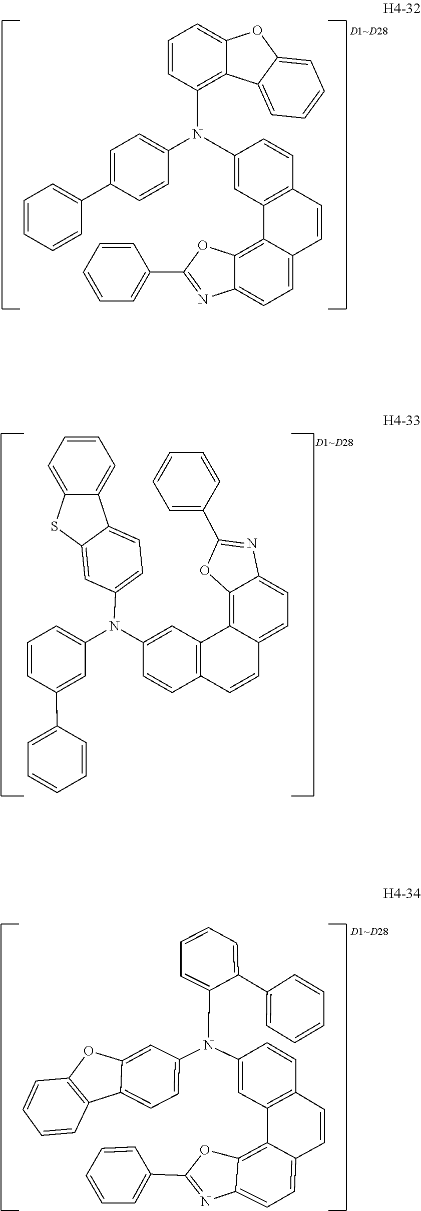

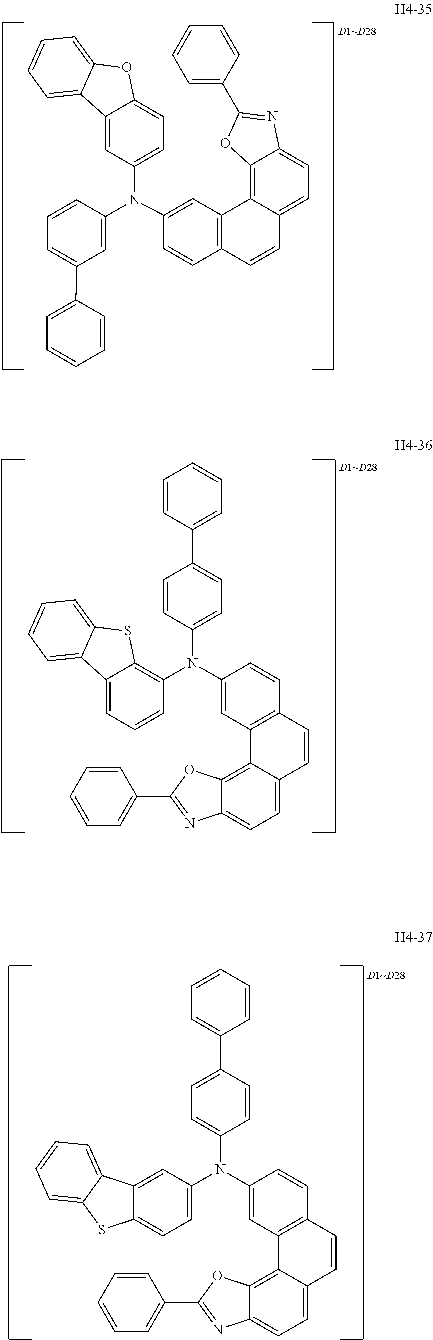

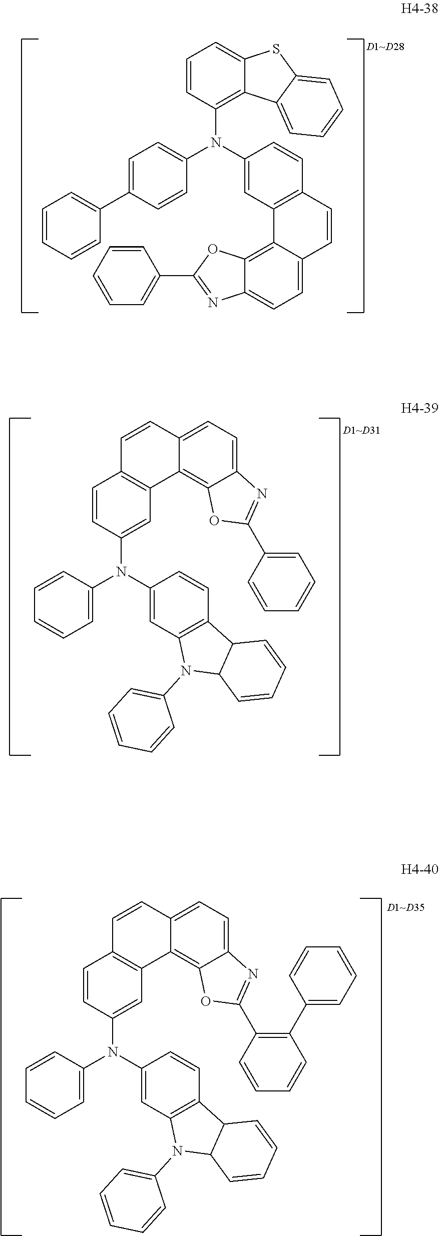

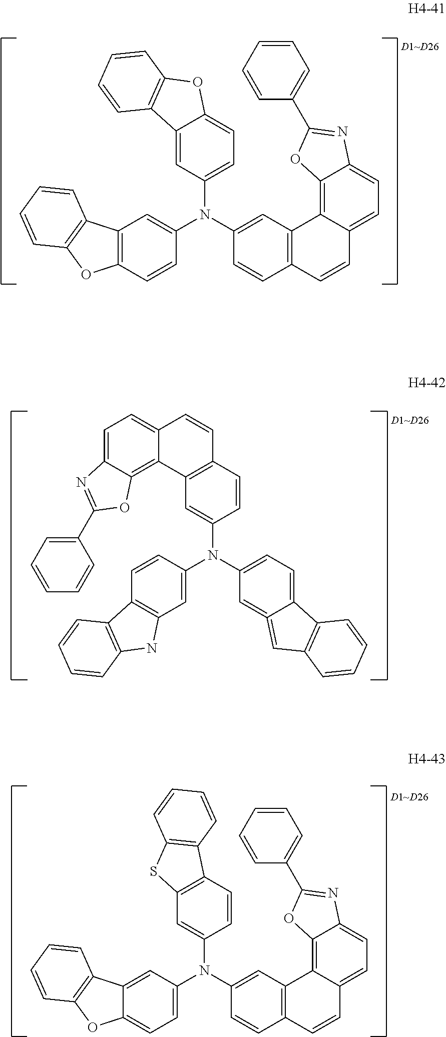

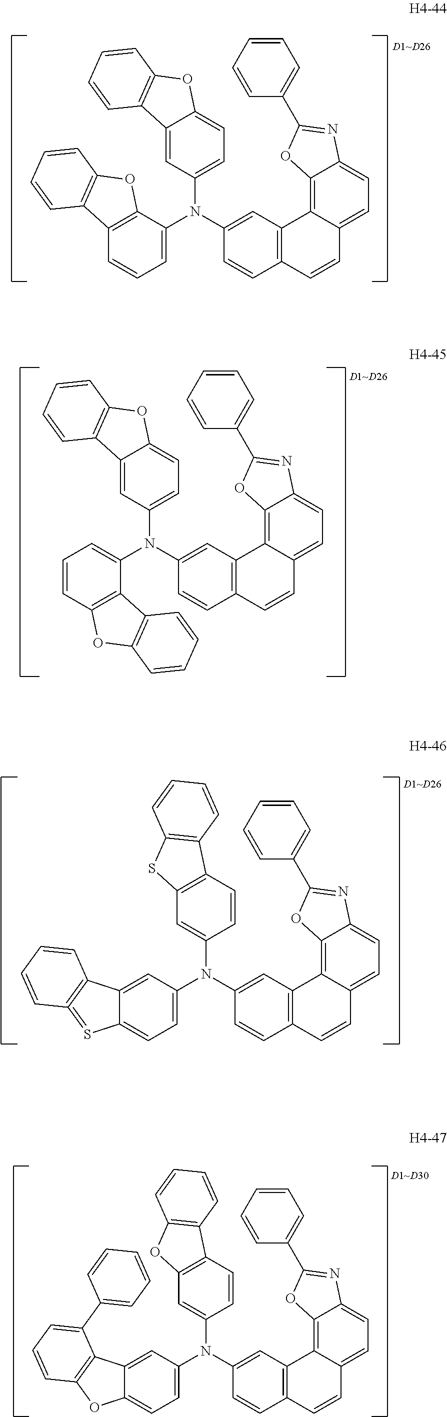

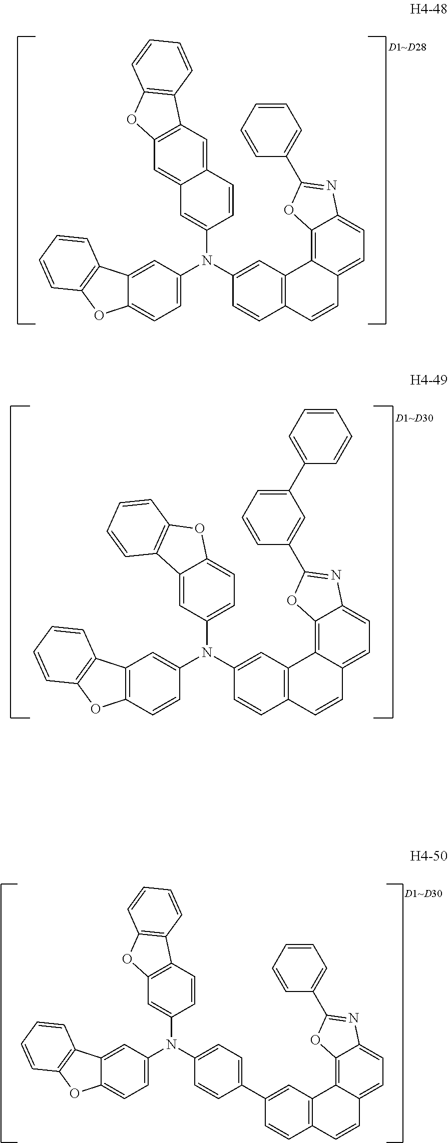

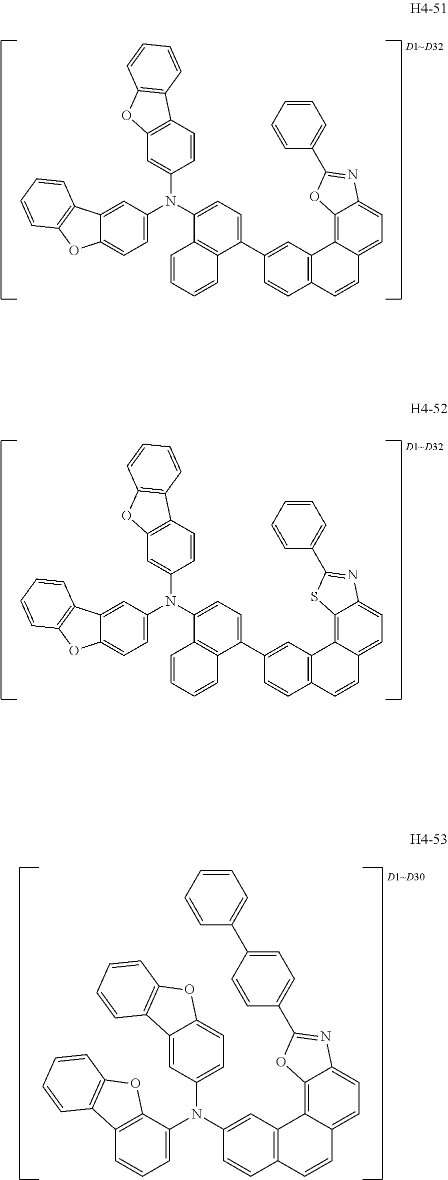

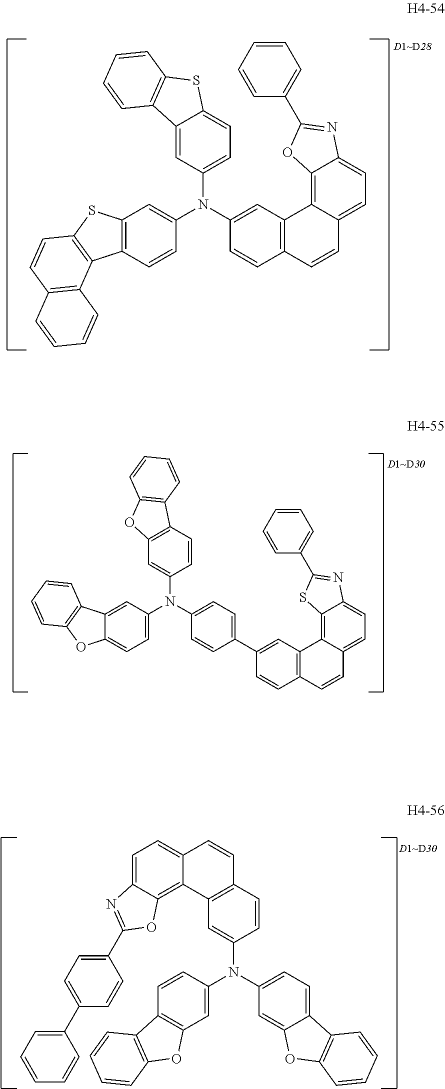

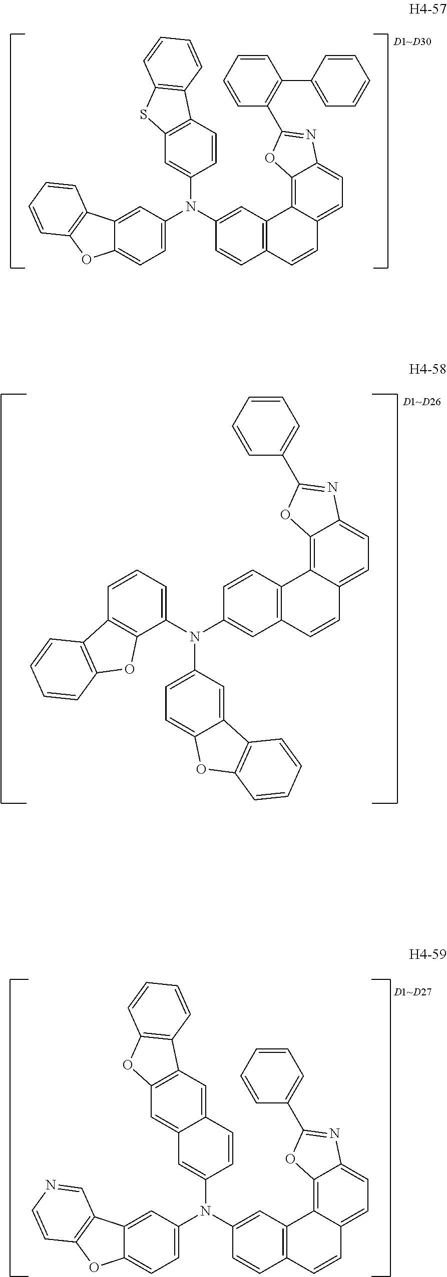

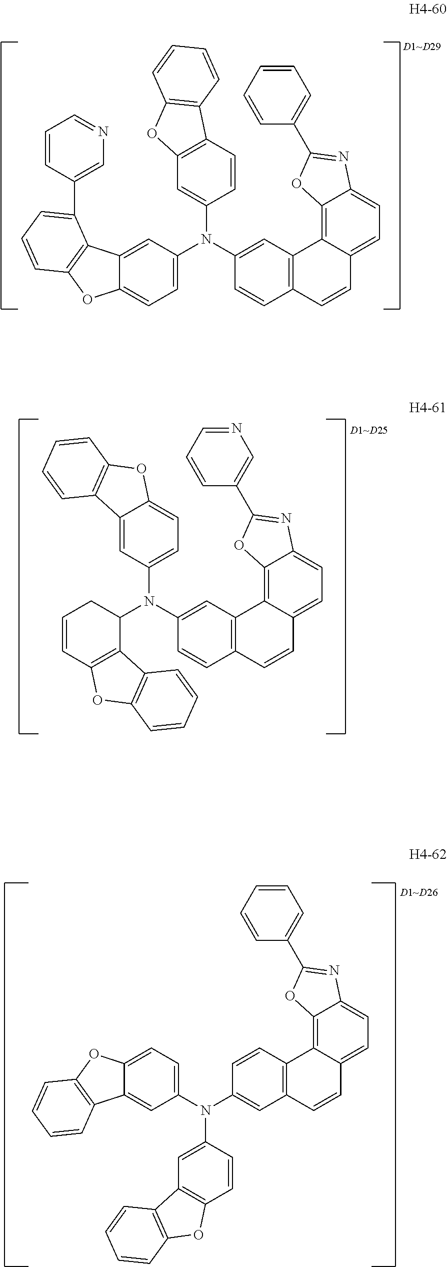

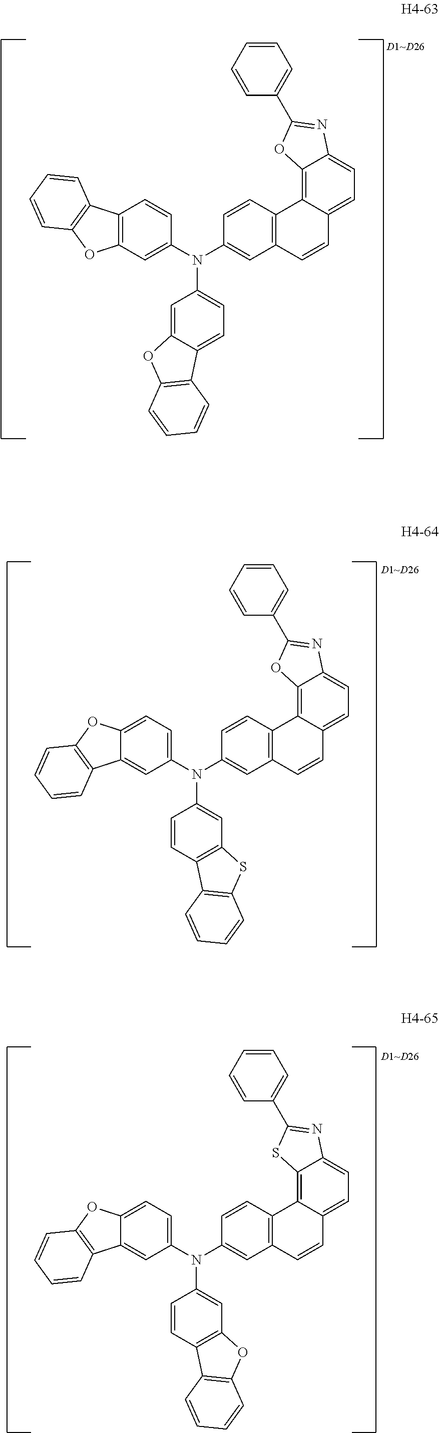

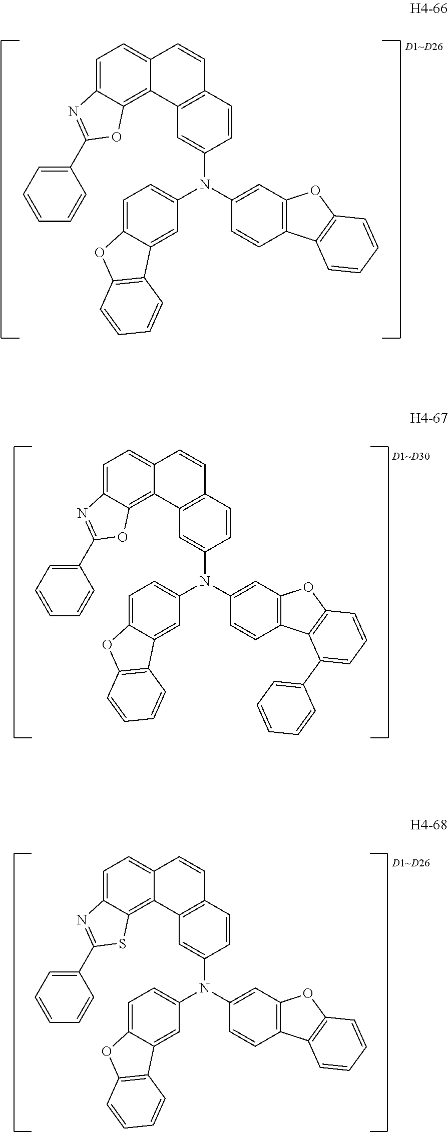

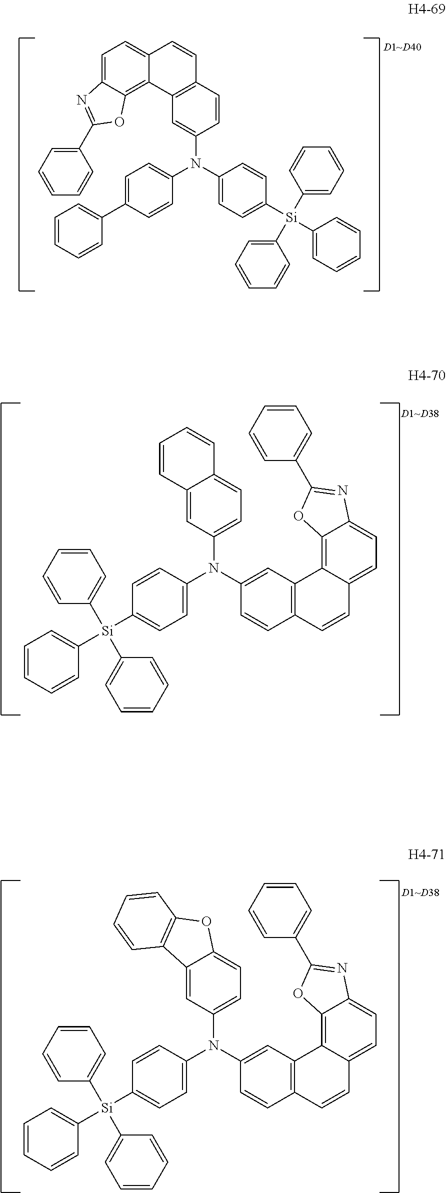

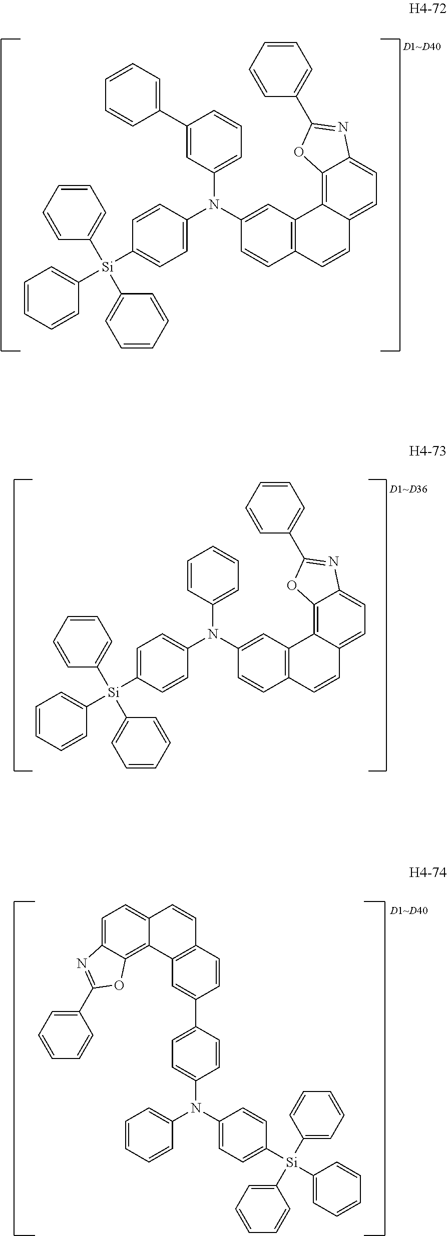

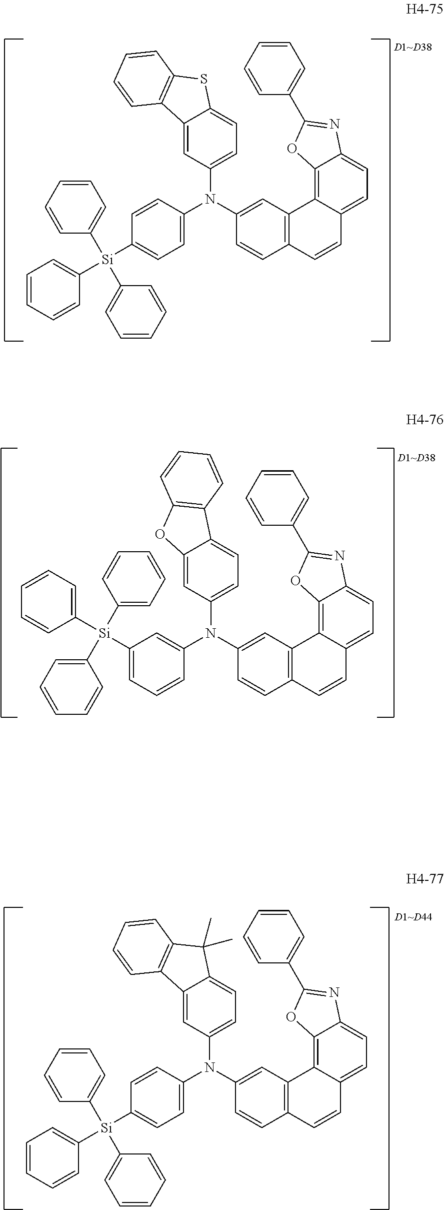

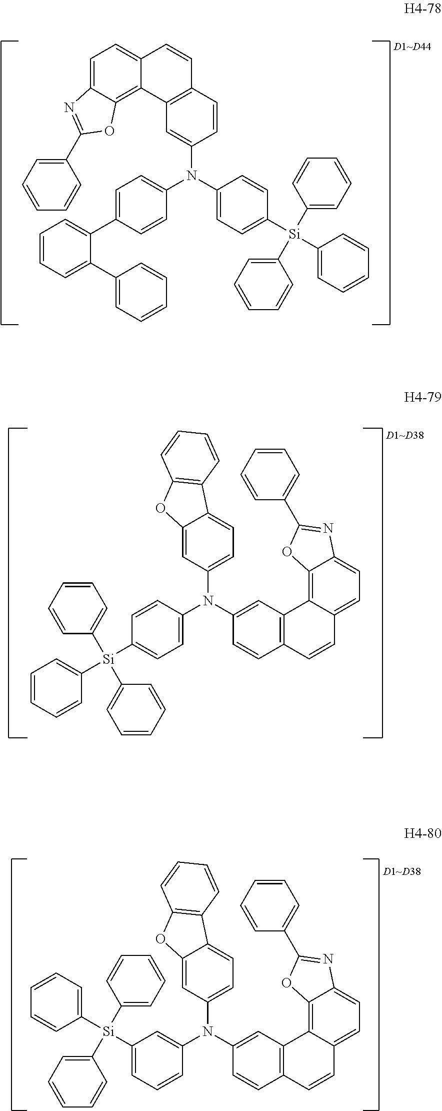

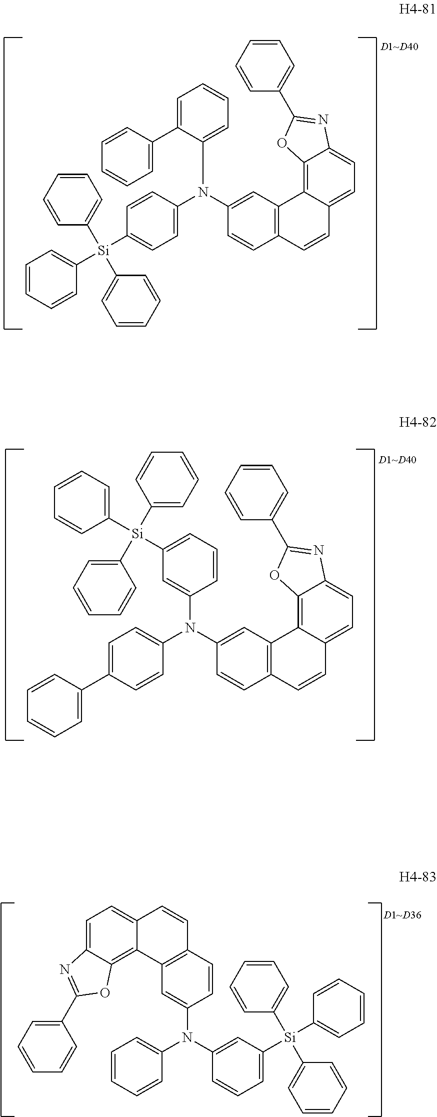

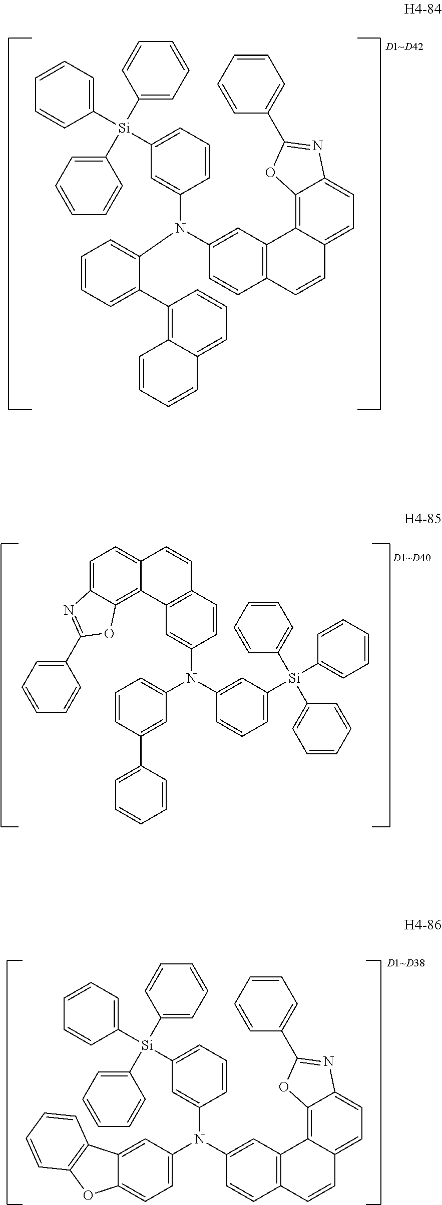

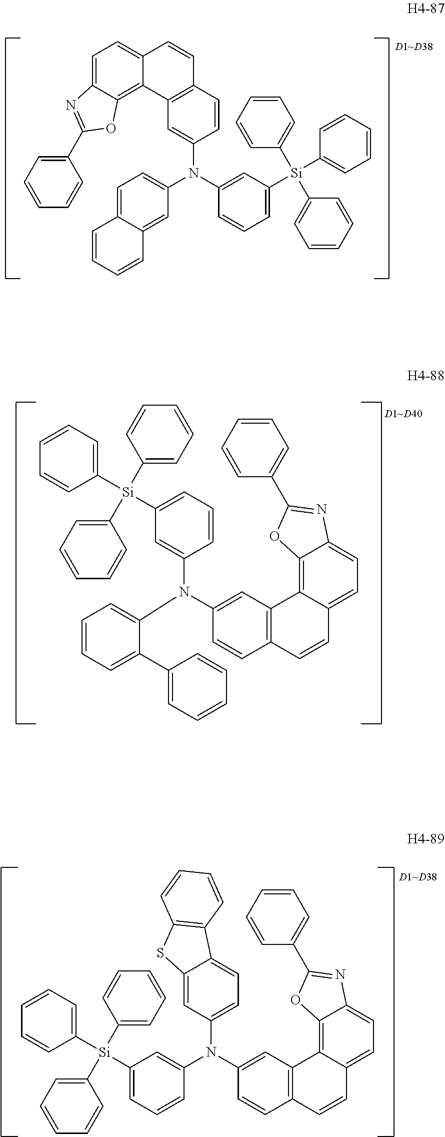

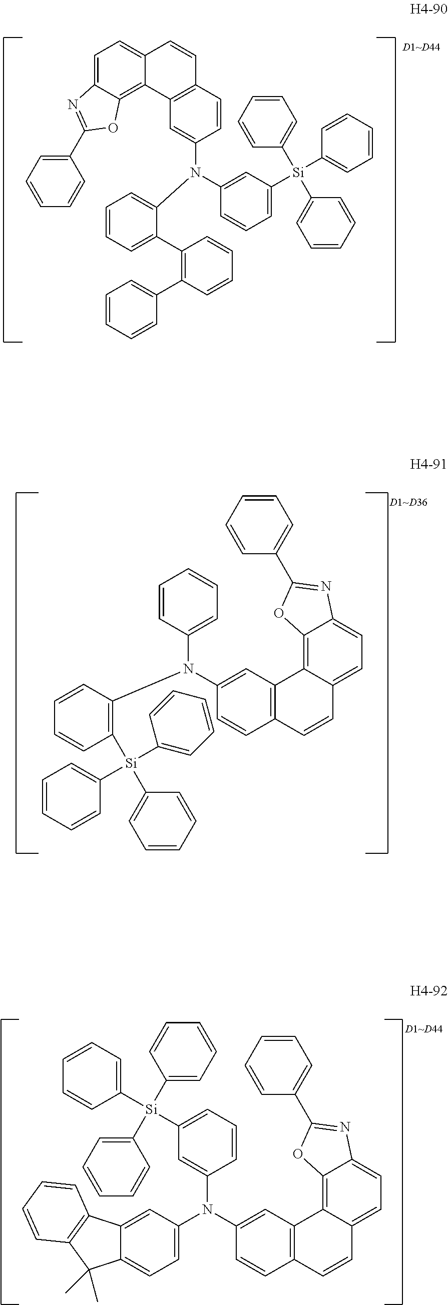

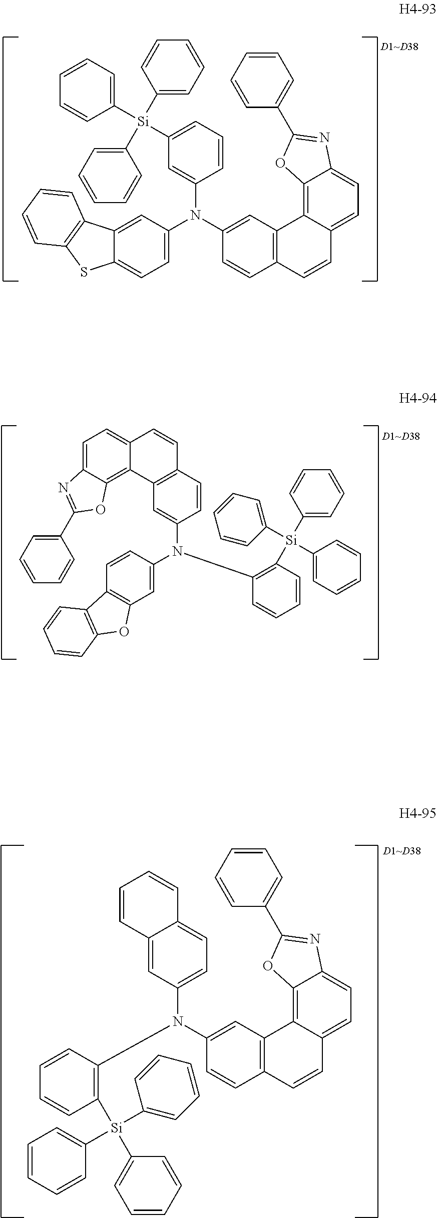

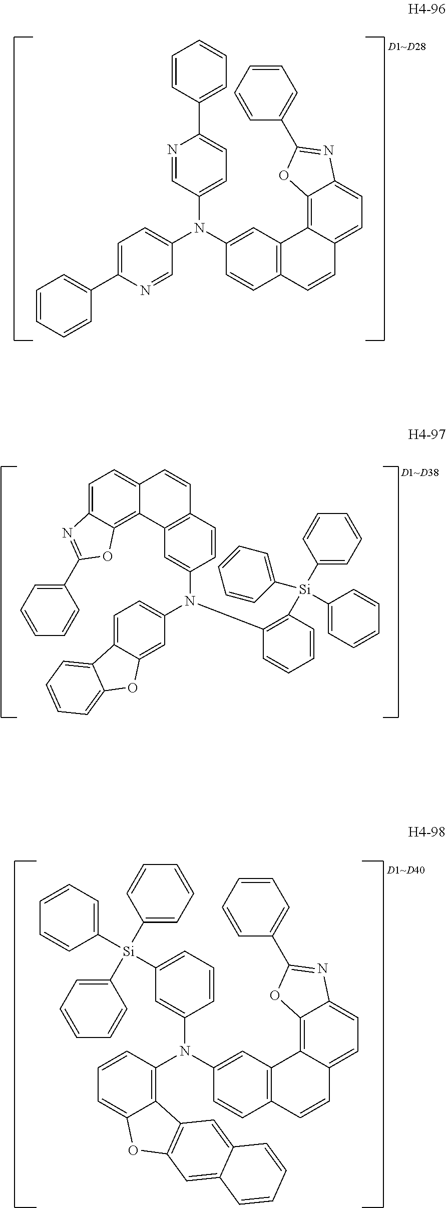

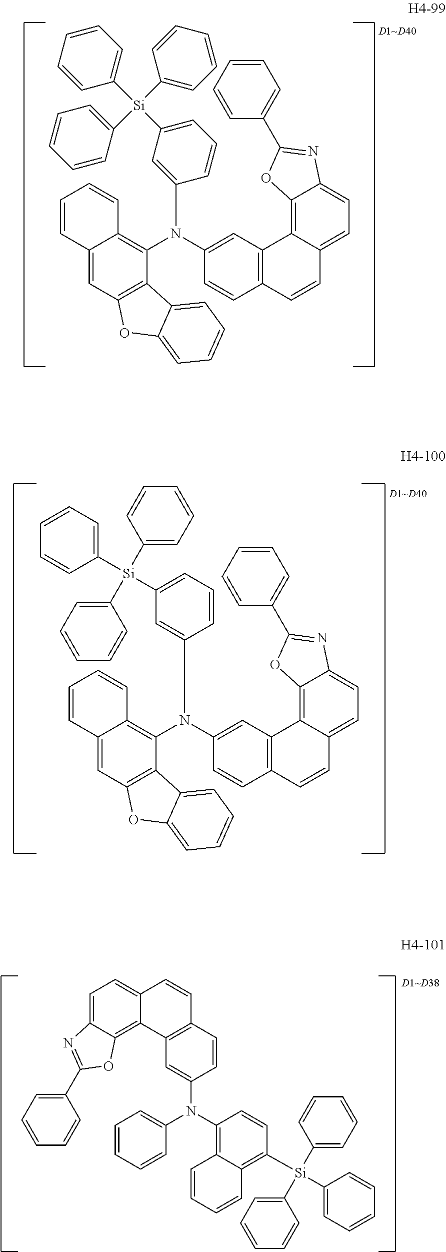

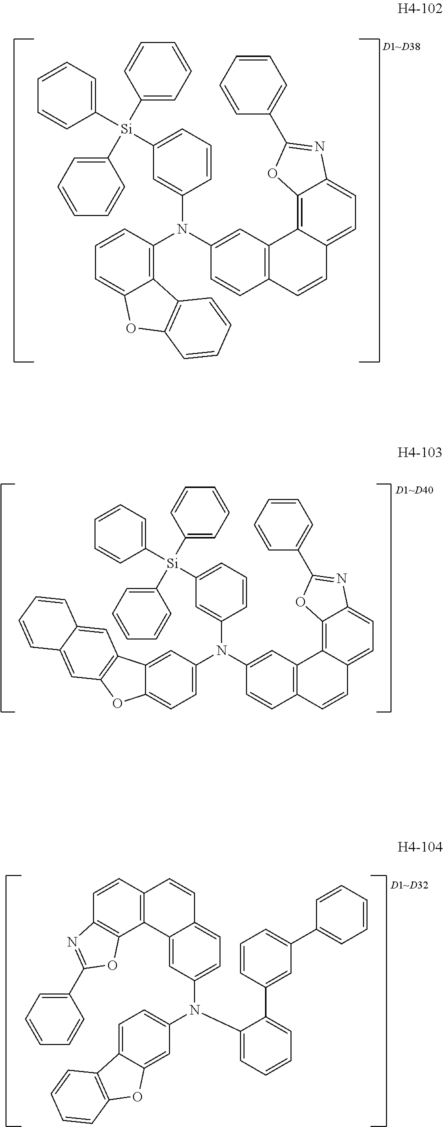

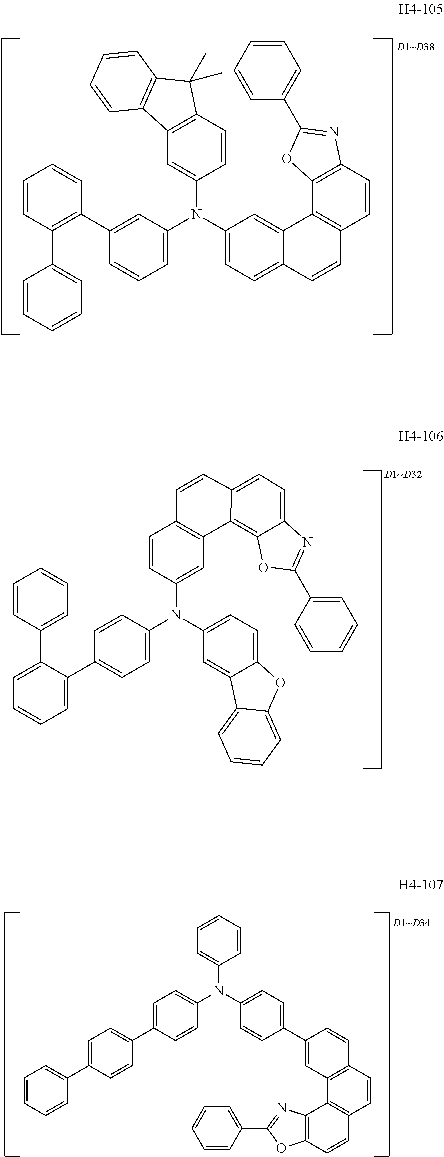

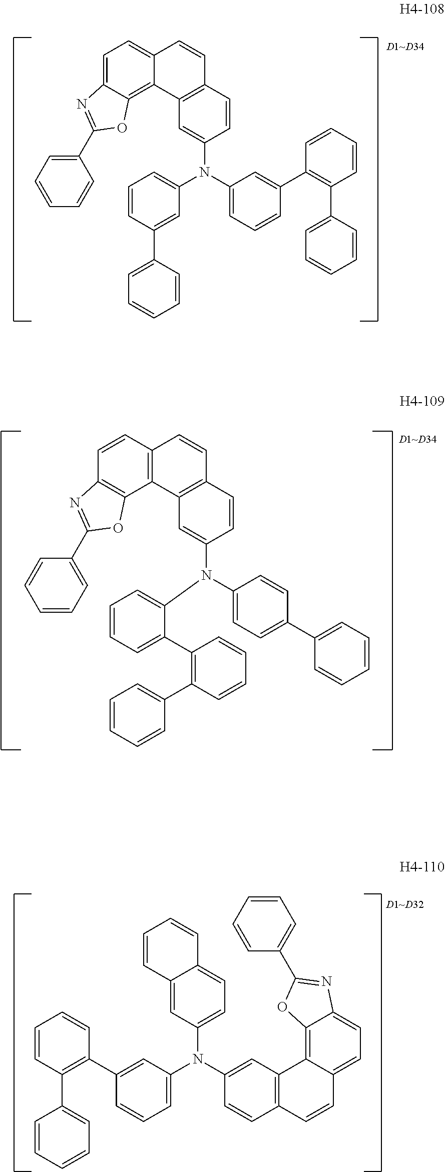

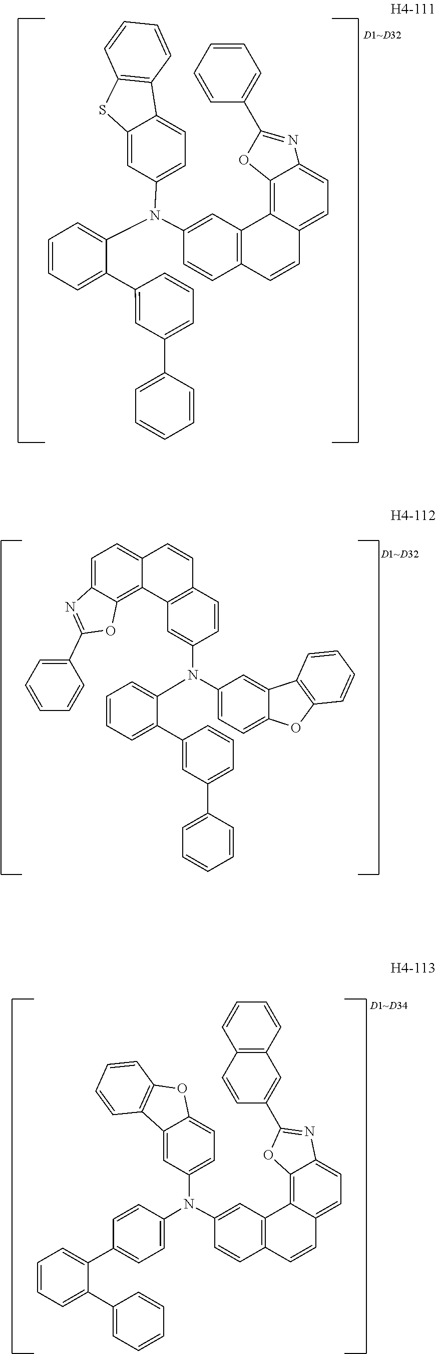

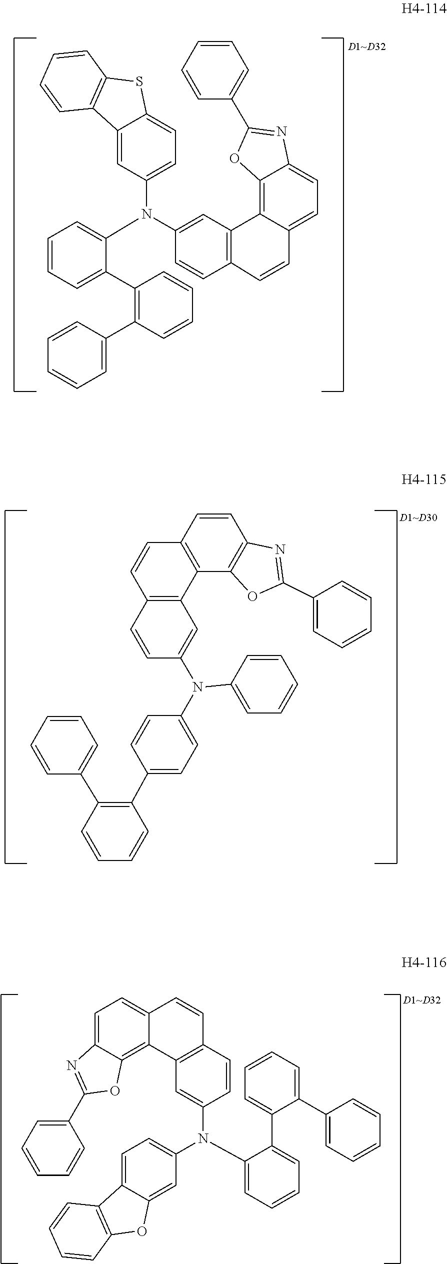

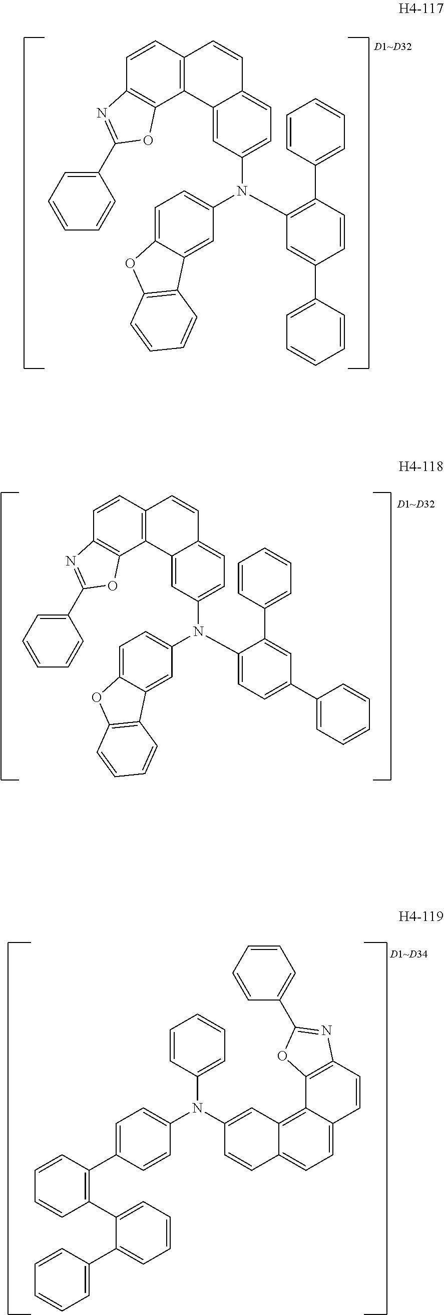

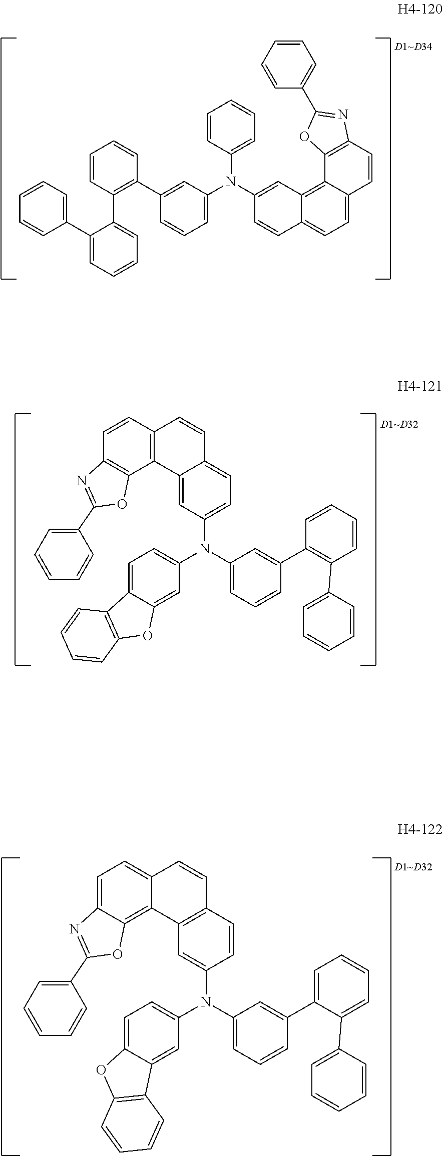

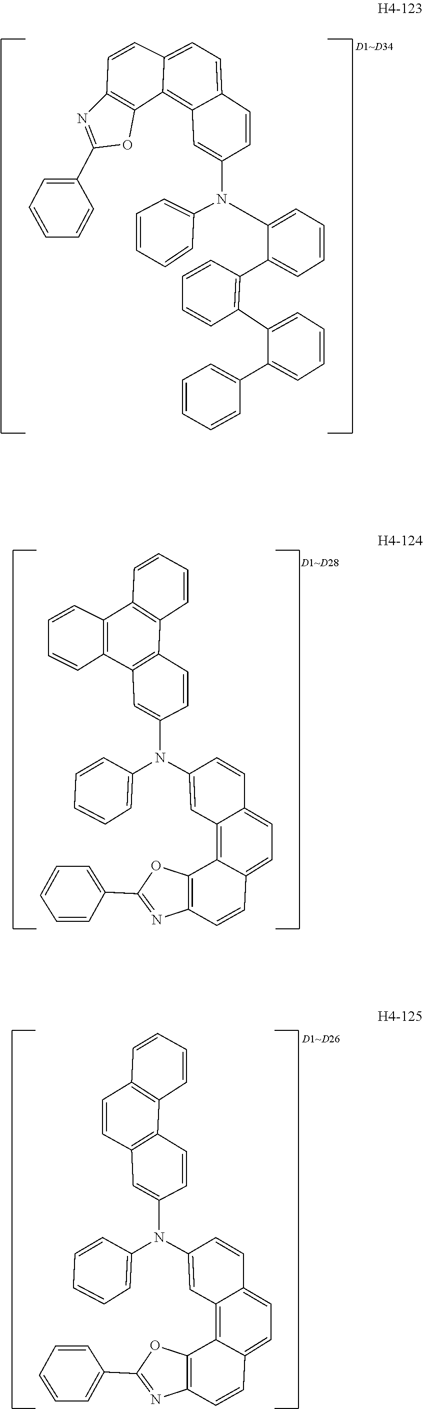

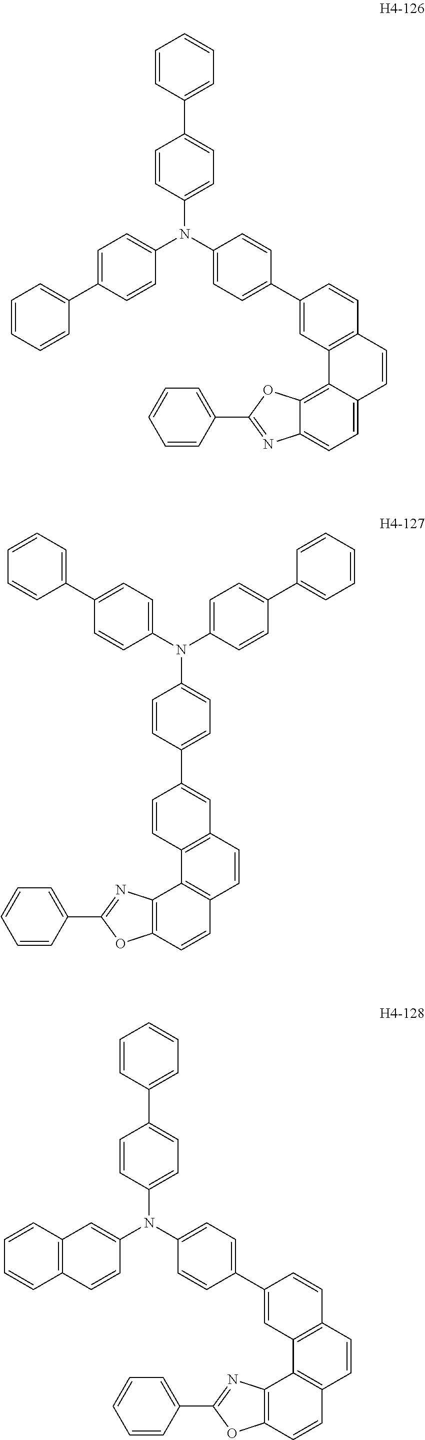 [SMILES: c1ccc(-c2ccc(N(c3ccc(-c4ccccc4)cc3)c3ccc(-c4ccc5ccc6ccc7nc(-c8ccccc8)oc7c6c5c4)cc3)cc2)cc1 ; c1ccc(-c2ccc(N(c3ccc(-c4ccccc4)cc3)c3ccc(-c4ccc5c(ccc6ccc7oc(-c8ccccc8)nc7c65)c4)cc3)cc2)cc1 ; c1ccc(-c2ccc(N(c3ccc(-c4ccc5ccc6ccc7oc(-c8ccccc8)nc7c6c5c4)cc3)c3ccc4ccccc4c3)cc2)cc1]

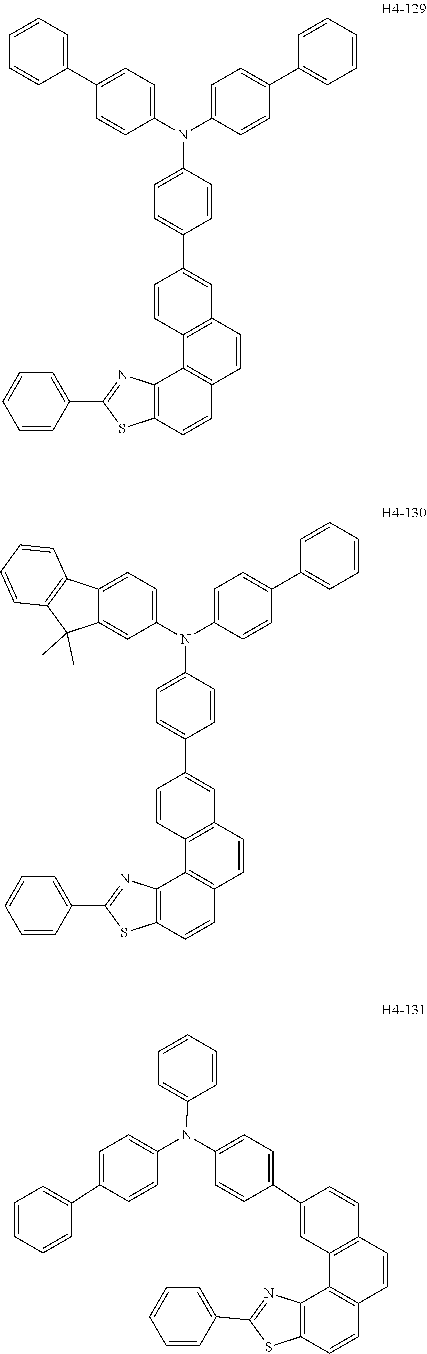 [SMILES: c1ccc(-c2ccc(N(c3ccc(-c4ccccc4)cc3)c3ccc(-c4ccc5c(ccc6ccc7sc(-c8ccccc8)nc7c65)c4)cc3)cc2)cc1 ; CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21 ; c1ccc(-c2ccc(N(c3ccccc3)c3ccc(-c4ccc5ccc6ccc7sc(-c8ccccc8)nc7c6c5c4)cc3)cc2)cc1]

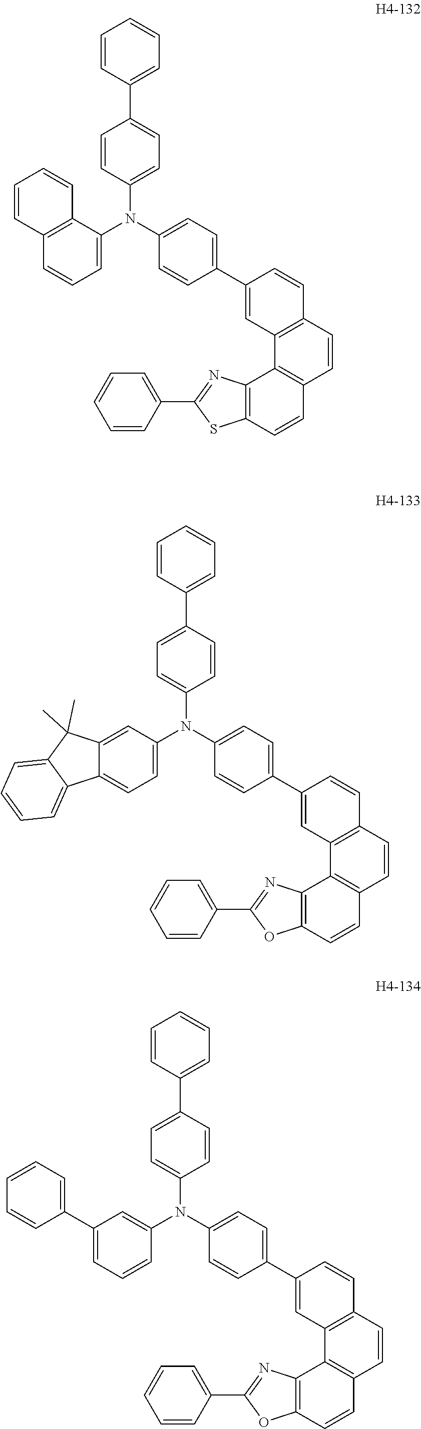 [SMILES: c1ccc(-c2ccc(N(c3ccc(-c4ccc5ccc6ccc7sc(-c8ccccc8)nc7c6c5c4)cc3)c3cccc4ccccc34)cc2)cc1 ; CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21 ; c1ccc(-c2ccc(N(c3ccc(-c4ccc5ccc6ccc7oc(-c8ccccc8)nc7c6c5c4)cc3)c3cccc(-c4ccccc4)c3)cc2)cc1]

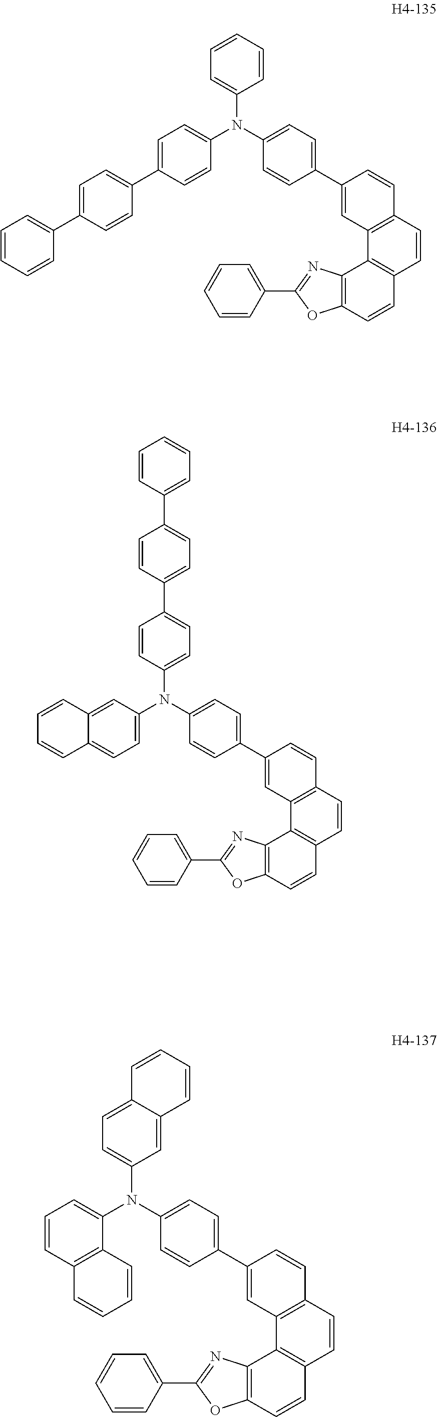 [SMILES: c1ccc(-c2ccc(-c3ccc(N(c4ccccc4)c4ccc(-c5ccc6ccc7ccc8oc(-c9ccccc9)nc8c7c6c5)cc4)cc3)cc2)cc1 ; c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1 ; c1ccc(-c2nc3c(ccc4ccc5ccc(-c6ccc(N(c7ccc8ccccc8c7)c7cccc8ccccc78)cc6)cc5c43)o2)cc1]

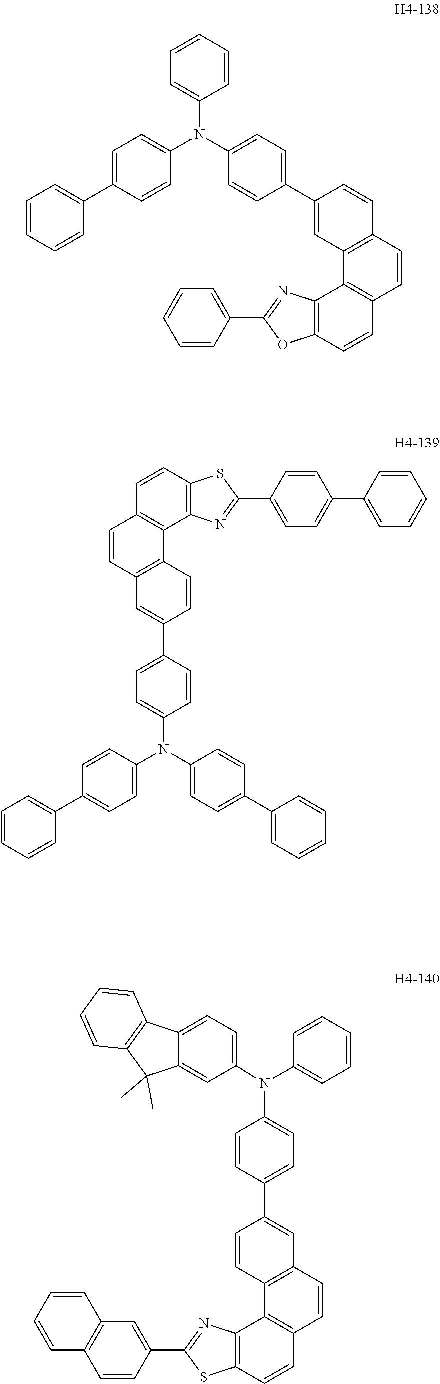 [SMILES: c1ccc(-c2ccc(N(c3ccccc3)c3ccc(-c4ccc5ccc6ccc7oc(-c8ccccc8)nc7c6c5c4)cc3)cc2)cc1 ; c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1 ; CC1(C)c2ccccc2-c2ccc(N(c3ccccc3)c3ccc(-c4ccc5c(ccc6ccc7sc(-c8ccc9ccccc9c8)nc7c65)c4)cc3)cc21]

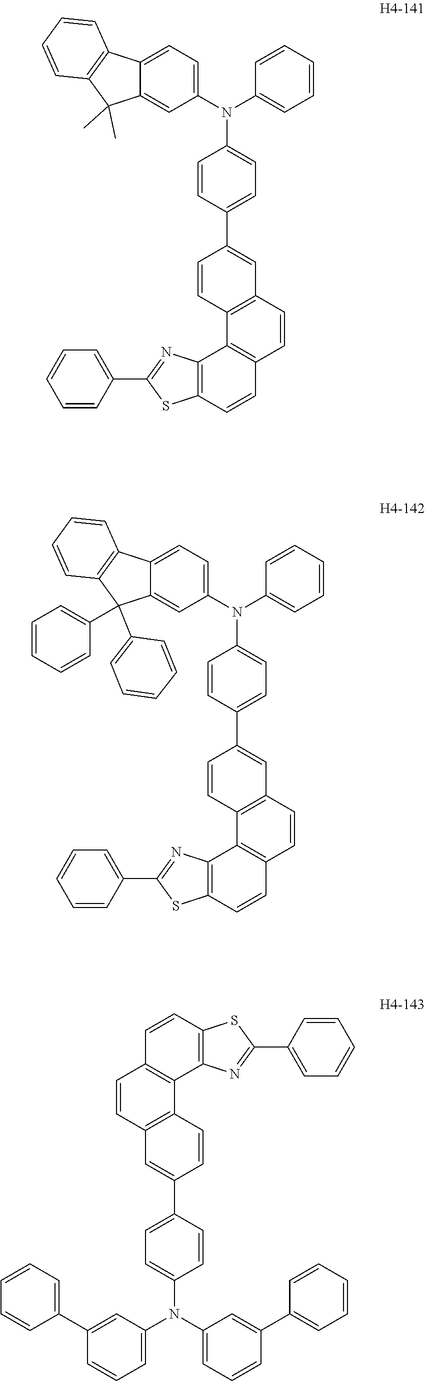 [SMILES: CC1(C)c2ccccc2-c2ccc(N(c3ccccc3)c3ccc(-c4ccc5c(ccc6ccc7sc(-c8ccccc8)nc7c65)c4)cc3)cc21 ; c1ccc(-c2nc3c(ccc4ccc5cc(-c6ccc(N(c7ccccc7)c7ccc8c(c7)C(c7ccccc7)(c7ccccc7)c7ccccc7-8)cc6)ccc5c43)s2)cc1 ; c1ccc(-c2cccc(N(c3ccc(-c4ccc5c(ccc6ccc7sc(-c8ccccc8)nc7c65)c4)cc3)c3cccc(-c4ccccc4)c3)c2)cc1]

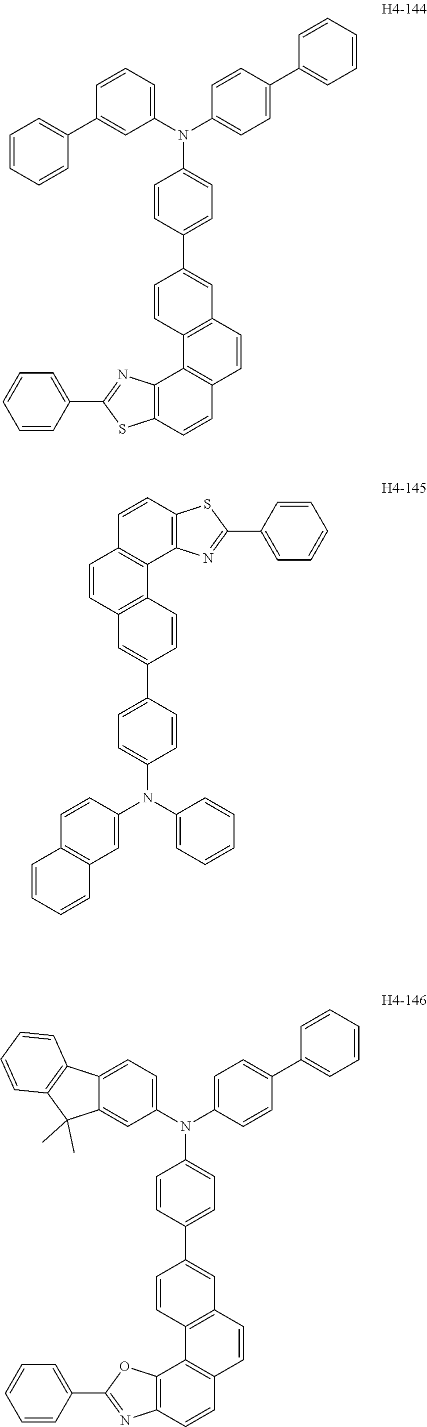 [SMILES: c1ccc(-c2ccc(N(c3ccc(-c4ccc5c(ccc6ccc7sc(-c8ccccc8)nc7c65)c4)cc3)c3cccc(-c4ccccc4)c3)cc2)cc1 ; c1ccc(-c2nc3c(ccc4ccc5cc(-c6ccc(N(c7ccccc7)c7ccc8ccccc8c7)cc6)ccc5c43)s2)cc1 ; CC1(C)c2ccccc2-c2ccc(N(c3ccc(-c4ccccc4)cc3)c3ccc(-c4ccc5c(ccc6ccc7nc(-c8ccccc8)oc7c65)c4)cc3)cc21]

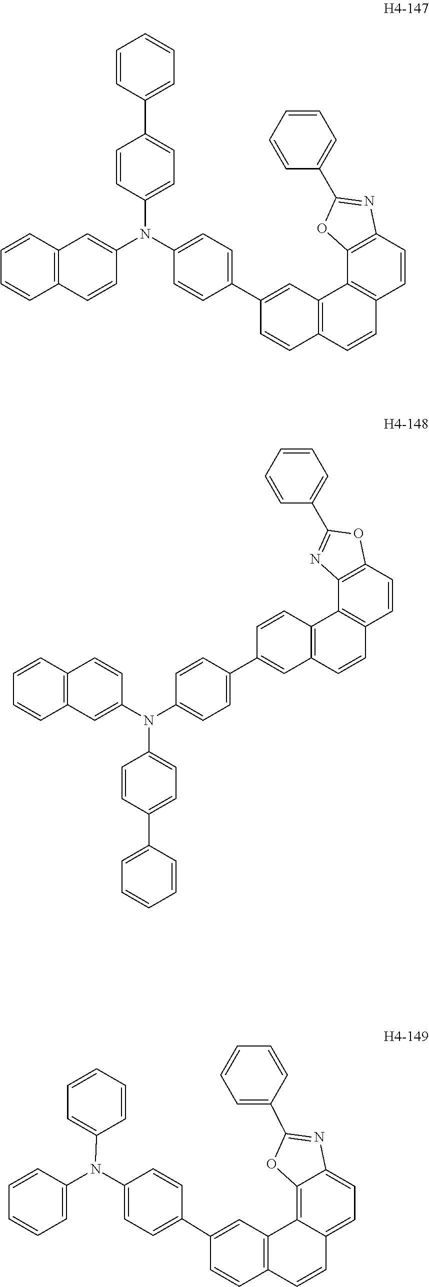 [SMILES: c1ccc(-c2ccc(N(c3ccc(-c4ccc5ccc6ccc7nc(-c8ccccc8)oc7c6c5c4)cc3)c3ccc4ccccc4c3)cc2)cc1 ; c1ccc(-c2ccc(N(c3ccc(-c4ccc5c(ccc6ccc7oc(-c8ccccc8)nc7c65)c4)cc3)c3ccc4ccccc4c3)cc2)cc1 ; c1ccc(-c2nc3ccc4ccc5ccc(-c6ccc(N(c7ccccc7)c7ccccc7)cc6)cc5c4c3o2)cc1]

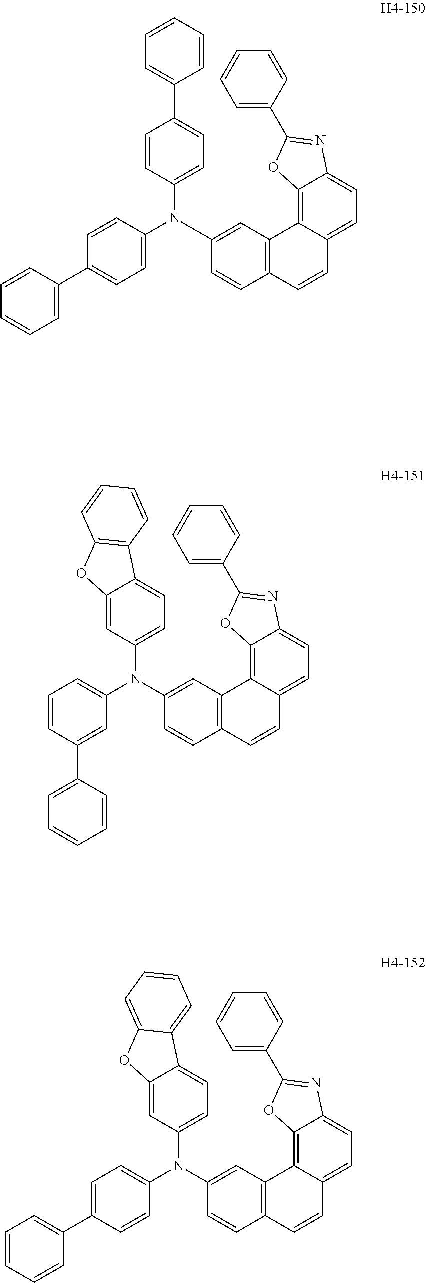 [SMILES: c1ccc(-c2ccc(N(c3ccc(-c4ccccc4)cc3)c3ccc4ccc5ccc6nc(-c7ccccc7)oc6c5c4c3)cc2)cc1 ; c1ccc(-c2cccc(N(c3ccc4c(c3)oc3ccccc34)c3ccc4ccc5ccc6nc(-c7ccccc7)oc6c5c4c3)c2)cc1 ; c1ccc(-c2ccc(N(c3ccc4c(c3)oc3ccccc34)c3ccc4ccc5ccc6nc(-c7ccccc7)oc6c5c4c3)cc2)cc1]

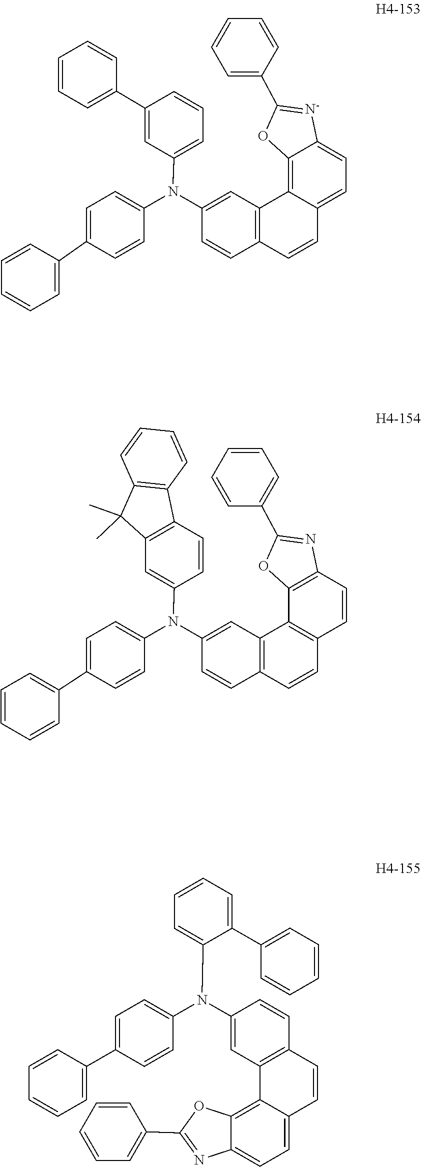

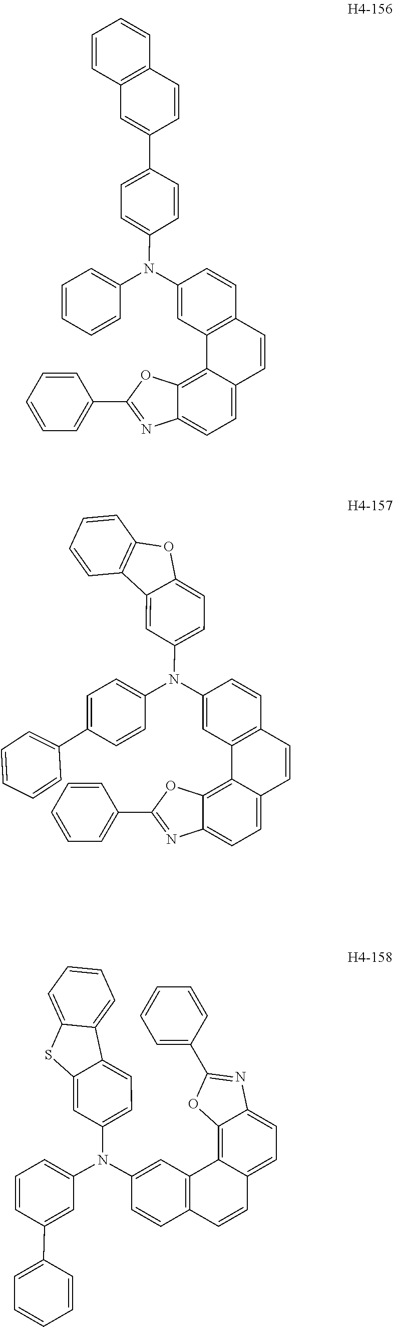 [SMILES: c1ccc(-c2nc3ccc4ccc5ccc(N(c6ccccc6)c6ccc(-c7ccc8ccccc8c7)cc6)cc5c4c3o2)cc1 ; c1ccc(-c2ccc(N(c3ccc4oc5ccccc5c4c3)c3ccc4ccc5ccc6nc(-c7ccccc7)oc6c5c4c3)cc2)cc1 ; c1ccc(-c2cccc(N(c3ccc4c(c3)sc3ccccc34)c3ccc4ccc5ccc6nc(-c7ccccc7)oc6c5c4c3)c2)cc1]

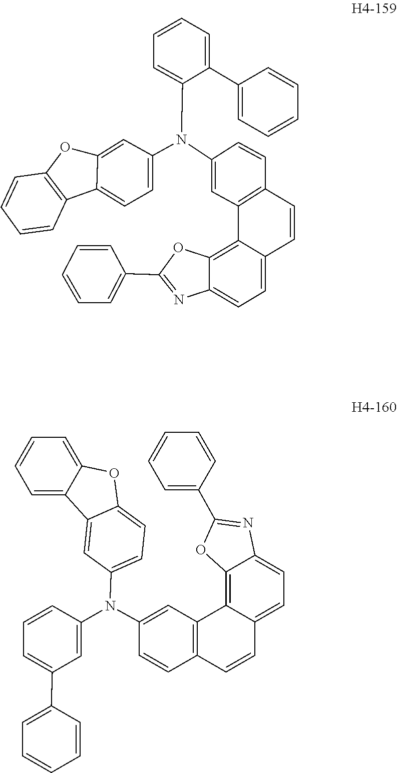 [SMILES: c1ccc(-c2nc3ccc4ccc5ccc(N(c6ccc7c(c6)oc6ccccc67)c6ccccc6-c6ccccc6)cc5c4c3o2)cc1 ; c1ccc(-c2cccc(N(c3ccc4oc5ccccc5c4c3)c3ccc4ccc5ccc6nc(-c7ccccc7)oc6c5c4c3)c2)cc1]

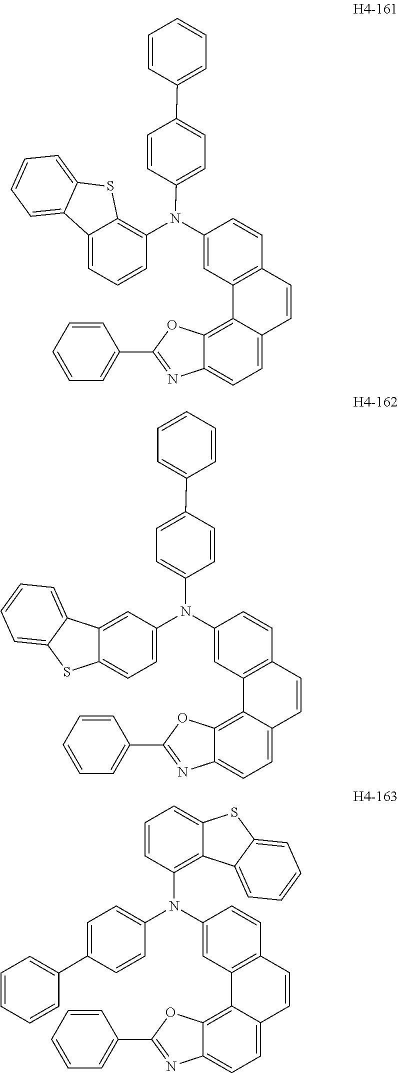

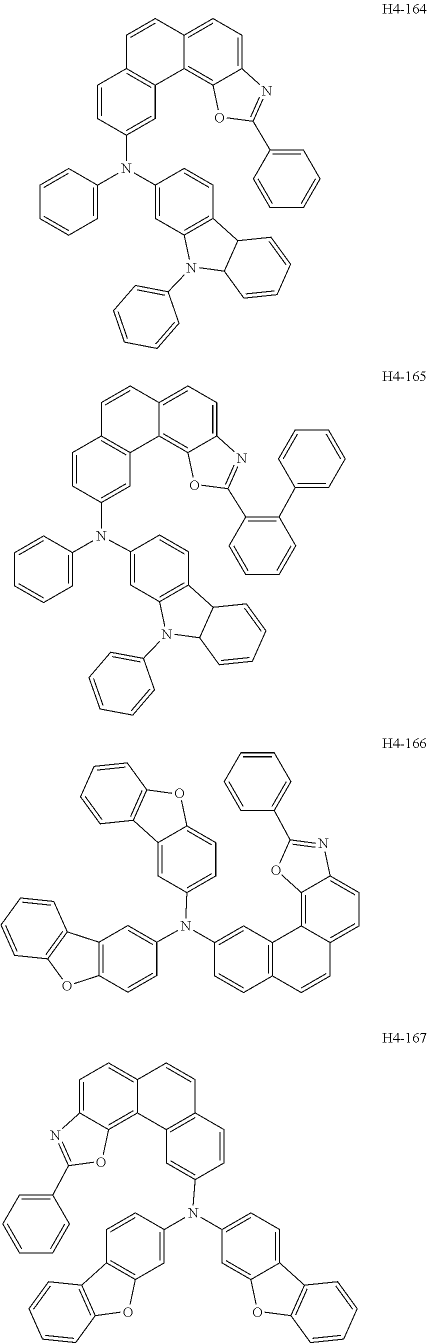

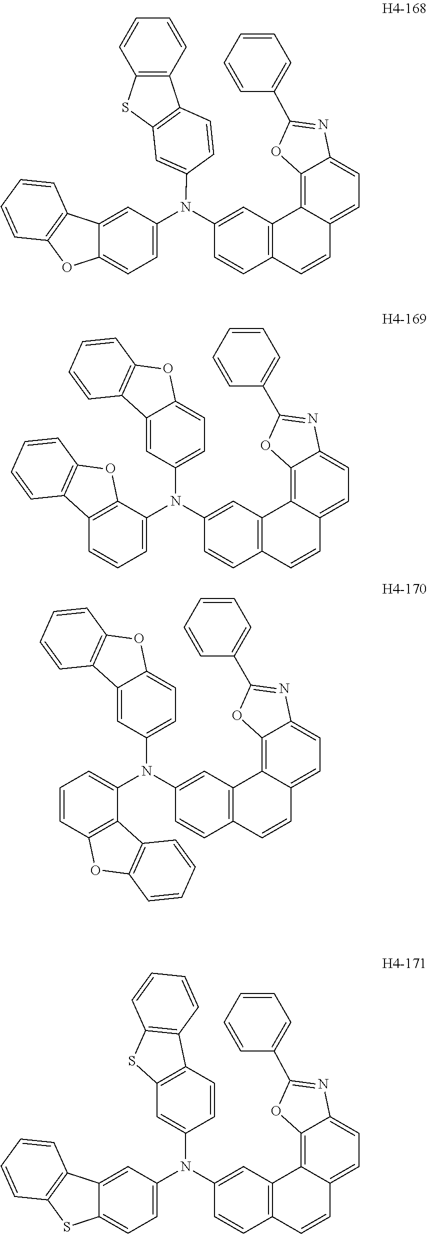

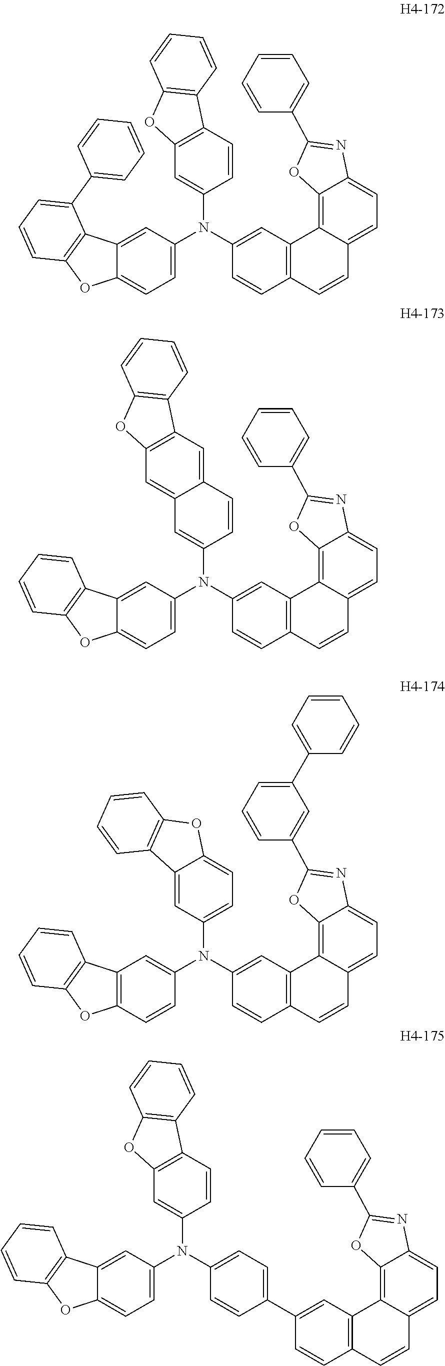

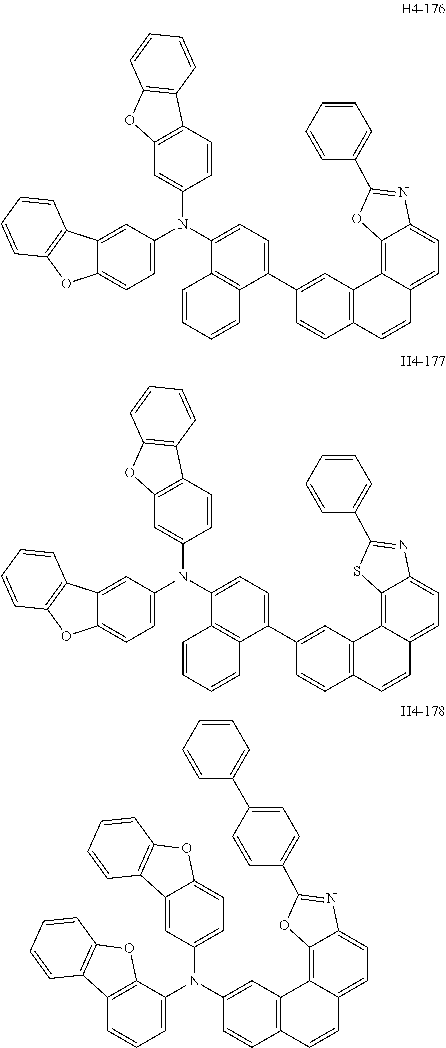

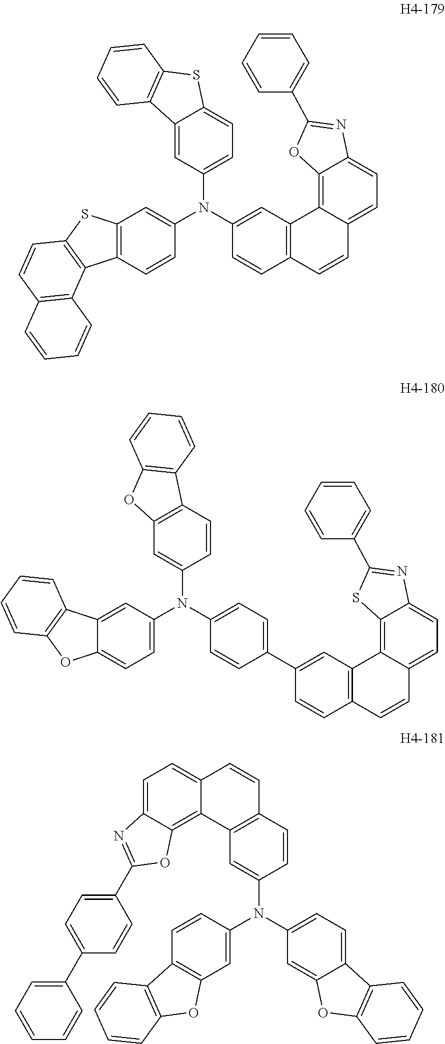 [SMILES: c1ccc(-c2nc3ccc4ccc5ccc(N(c6ccc7c(c6)sc6ccc8ccccc8c67)c6ccc7sc8ccccc8c7c6)cc5c4c3o2)cc1 ; c1ccc(-c2nc3ccc4ccc5ccc(-c6ccc(N(c7ccc8c(c7)oc7ccccc78)c7ccc8oc9ccccc9c8c7)cc6)cc5c4c3s2)cc1 ; c1ccc(-c2ccc(-c3nc4ccc5ccc6ccc(N(c7ccc8c(c7)oc7ccccc78)c7ccc8c(c7)oc7ccccc78)cc6c5c4o3)cc2)cc1]

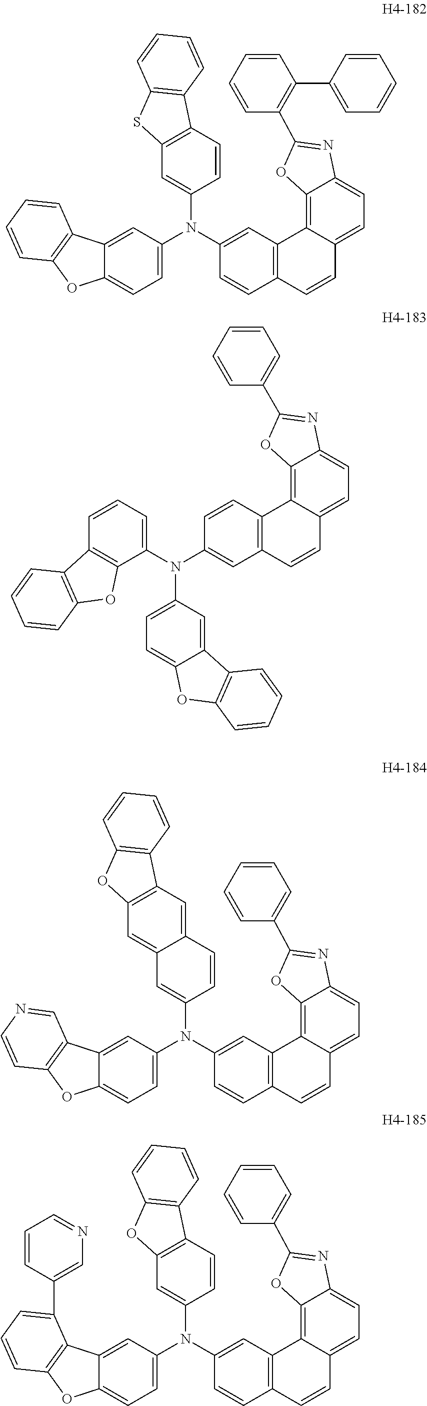

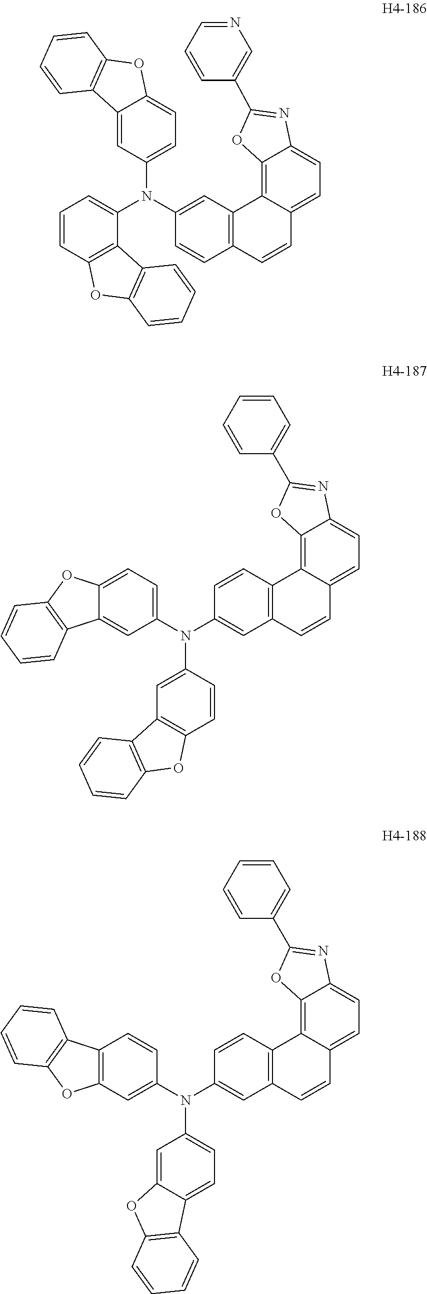 [SMILES: c1cncc(-c2nc3ccc4ccc5ccc(N(c6ccc7oc8ccccc8c7c6)c6cccc7oc8ccccc8c67)cc5c4c3o2)c1 ; c1ccc(-c2nc3ccc4ccc5cc(N(c6ccc7oc8ccccc8c7c6)c6ccc7oc8ccccc8c7c6)ccc5c4c3o2)cc1 ; c1ccc(-c2nc3ccc4ccc5cc(N(c6ccc7c(c6)oc6ccccc67)c6ccc7c(c6)oc6ccccc67)ccc5c4c3o2)cc1]

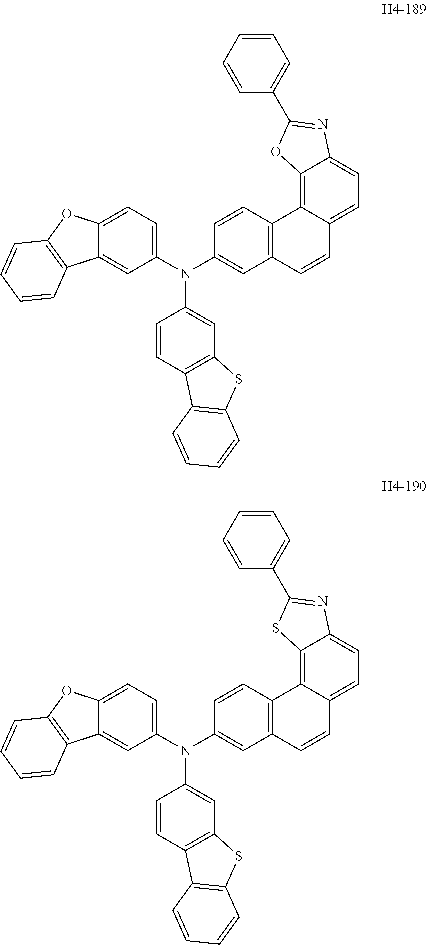 [SMILES: c1ccc(-c2nc3ccc4ccc5cc(N(c6ccc7c(c6)sc6ccccc67)c6ccc7oc8ccccc8c7c6)ccc5c4c3o2)cc1 ; c1ccc(-c2nc3ccc4ccc5cc(N(c6ccc7c(c6)sc6ccccc67)c6ccc7oc8ccccc8c7c6)ccc5c4c3s2)cc1]

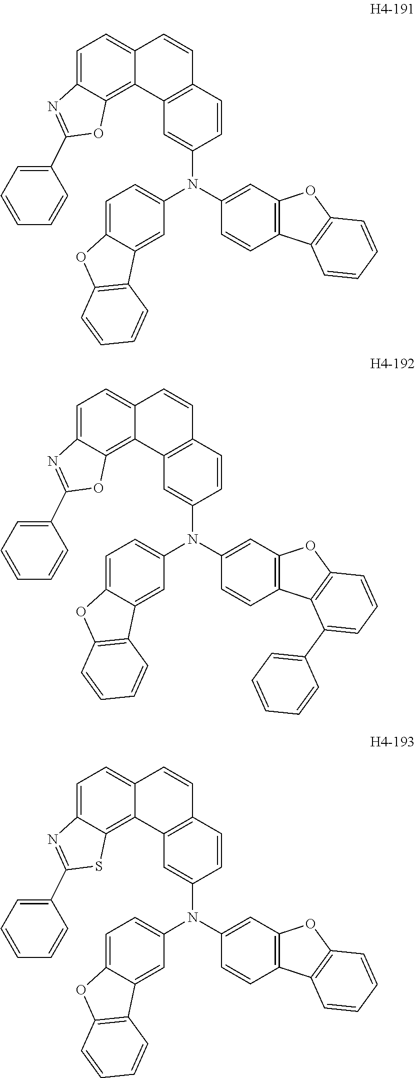

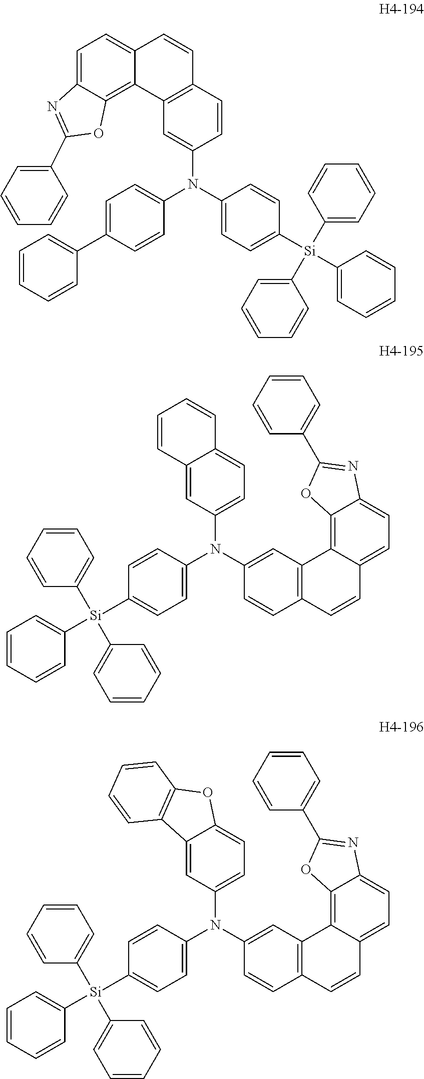

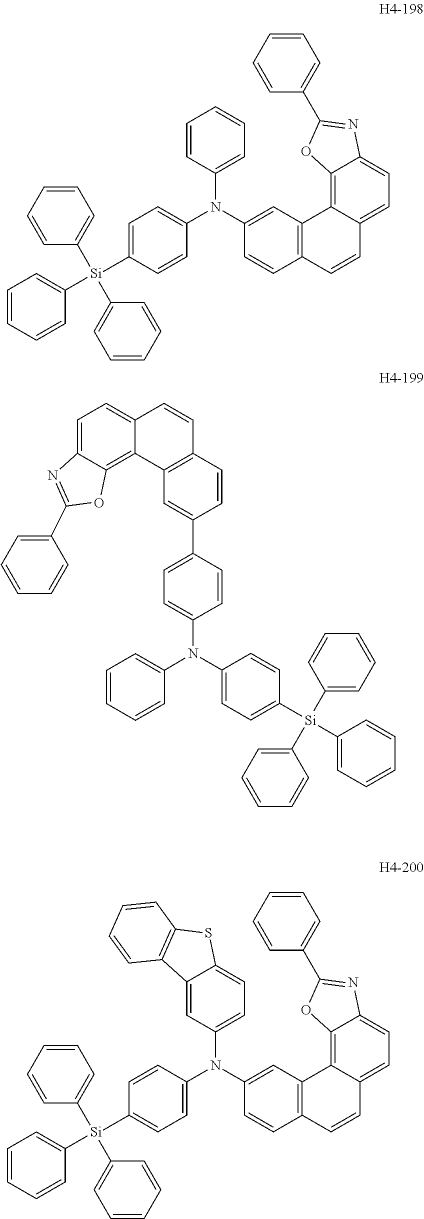

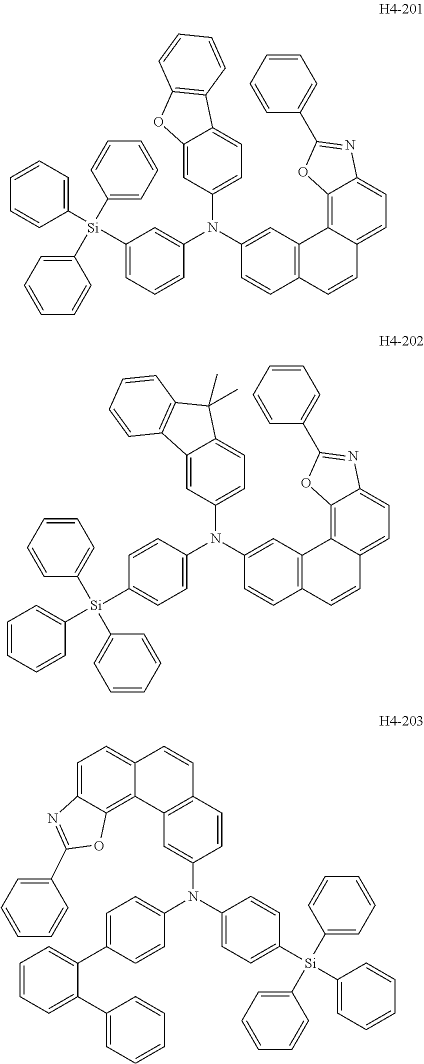 [SMILES: c1ccc(-c2nc3ccc4ccc5ccc(N(c6cccc([Si](c7ccccc7)(c7ccccc7)c7ccccc7)c6)c6ccc7c(c6)oc6ccccc67)cc5c4c3o2)cc1 ; CC1(C)c2ccccc2-c2cc(N(c3ccc([Si](c4ccccc4)(c4ccccc4)c4ccccc4)cc3)c3ccc4ccc5ccc6nc(-c7ccccc7)oc6c5c4c3)ccc21 ; c1ccc(-c2nc3ccc4ccc5ccc(N(c6ccc(-c7ccccc7-c7ccccc7)cc6)c6ccc([Si](c7ccccc7)(c7ccccc7)c7ccccc7)cc6)cc5c4c3o2)cc1]

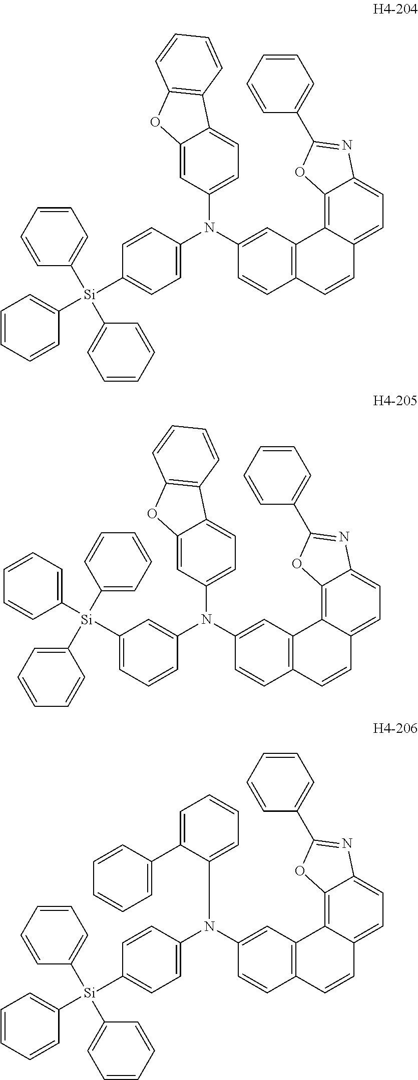 [SMILES: c1ccc(-c2nc3ccc4ccc5ccc(N(c6ccc([Si](c7ccccc7)(c7ccccc7)c7ccccc7)cc6)c6ccc7c(c6)oc6ccccc67)cc5c4c3o2)cc1 ; c1ccc(-c2nc3ccc4ccc5ccc(N(c6cccc([Si](c7ccccc7)(c7ccccc7)c7ccccc7)c6)c6ccc7c(c6)oc6ccccc67)cc5c4c3o2)cc1 ; c1ccc(-c2nc3ccc4ccc5ccc(N(c6ccc([Si](c7ccccc7)(c7ccccc7)c7ccccc7)cc6)c6ccccc6-c6ccccc6)cc5c4c3o2)cc1]

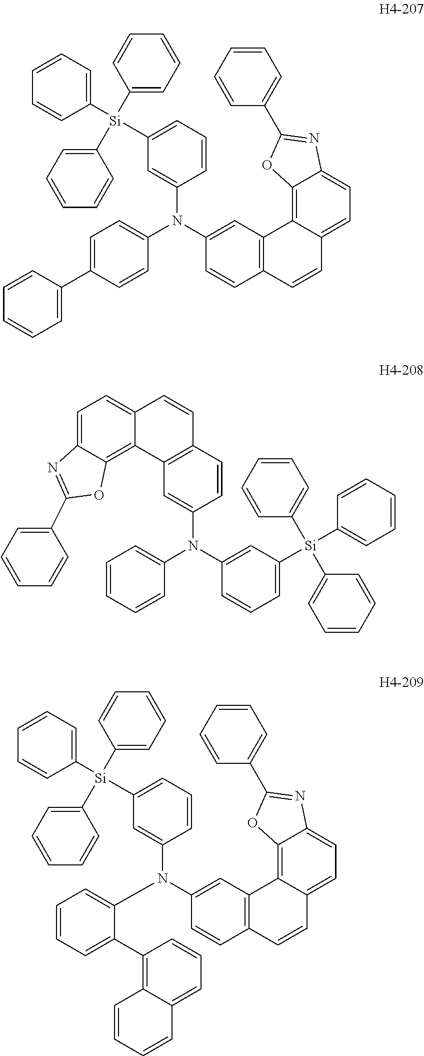 [SMILES: c1ccc(-c2ccc(N(c3cccc([Si](c4ccccc4)(c4ccccc4)c4ccccc4)c3)c3ccc4ccc5ccc6nc(-c7ccccc7)oc6c5c4c3)cc2)cc1 ; c1ccc(-c2nc3ccc4ccc5ccc(N(c6ccccc6)c6cccc([Si](c7ccccc7)(c7ccccc7)c7ccccc7)c6)cc5c4c3o2)cc1 ; c1ccc(-c2nc3ccc4ccc5ccc(N(c6cccc([Si](c7ccccc7)(c7ccccc7)c7ccccc7)c6)c6ccccc6-c6cccc7ccccc67)cc5c4c3o2)cc1]

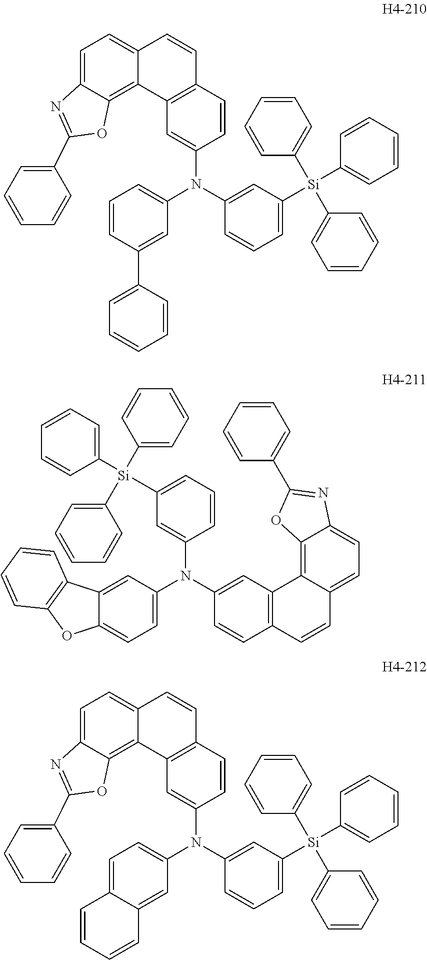

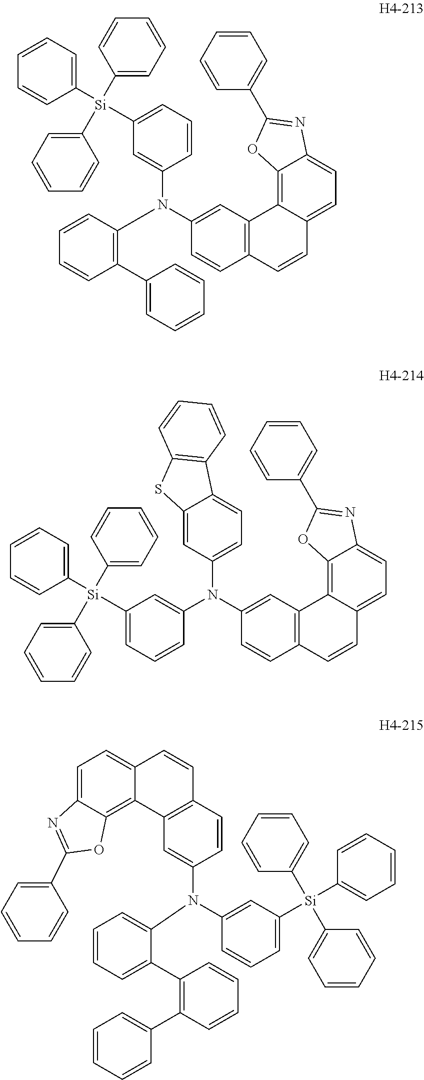 [SMILES: c1ccc(-c2nc3ccc4ccc5ccc(N(c6cccc([Si](c7ccccc7)(c7ccccc7)c7ccccc7)c6)c6ccccc6-c6ccccc6)cc5c4c3o2)cc1 ; c1ccc(-c2nc3ccc4ccc5ccc(N(c6cccc([Si](c7ccccc7)(c7ccccc7)c7ccccc7)c6)c6ccc7c(c6)sc6ccccc67)cc5c4c3o2)cc1 ; c1ccc(-c2nc3ccc4ccc5ccc(N(c6cccc([Si](c7ccccc7)(c7ccccc7)c7ccccc7)c6)c6ccccc6-c6ccccc6-c6ccccc6)cc5c4c3o2)cc1]

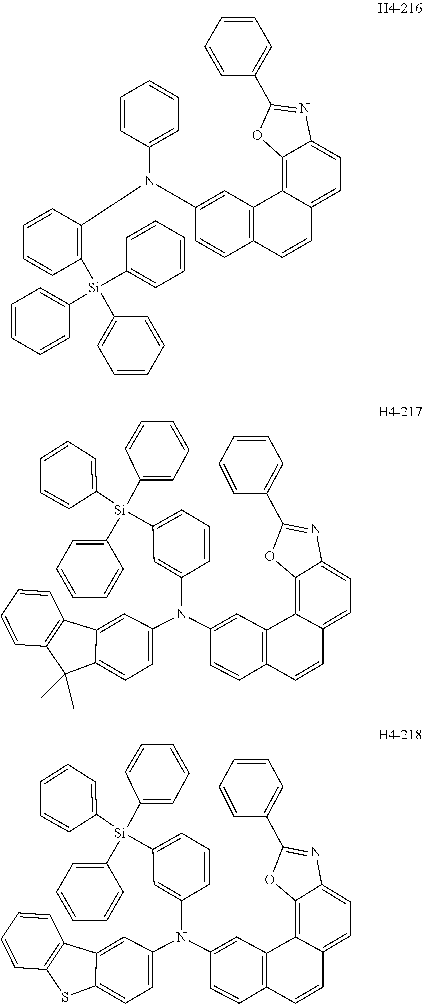 [SMILES: c1ccc(-c2nc3ccc4ccc5ccc(N(c6ccccc6)c6ccccc6[Si](c6ccccc6)(c6ccccc6)c6ccccc6)cc5c4c3o2)cc1 ; CC1(C)c2ccccc2-c2cc(N(c3cccc([Si](c4ccccc4)(c4ccccc4)c4ccccc4)c3)c3ccc4ccc5ccc6nc(-c7ccccc7)oc6c5c4c3)ccc21 ; c1ccc(-c2nc3ccc4ccc5ccc(N(c6cccc([Si](c7ccccc7)(c7ccccc7)c7ccccc7)c6)c6ccc7sc8ccccc8c7c6)cc5c4c3o2)cc1]

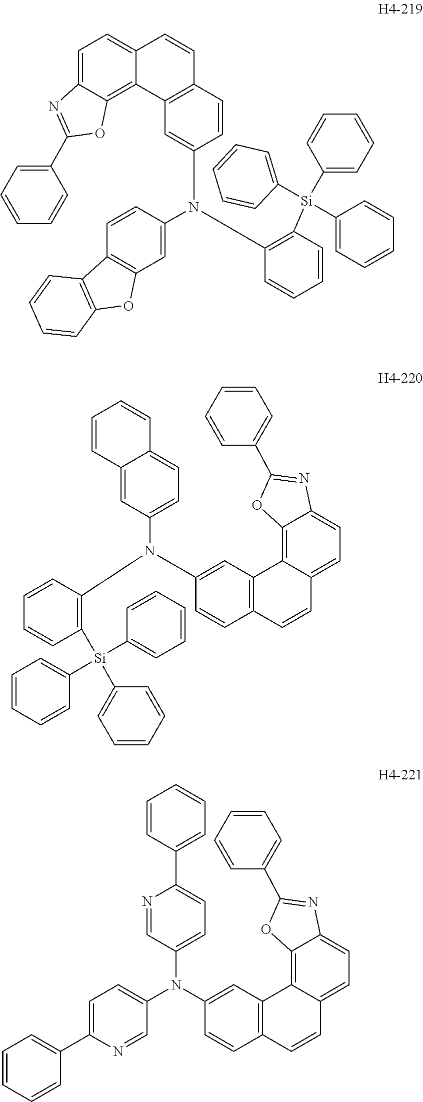 [SMILES: c1ccc(-c2nc3ccc4ccc5ccc(N(c6ccc7c(c6)oc6ccccc67)c6ccccc6[Si](c6ccccc6)(c6ccccc6)c6ccccc6)cc5c4c3o2)cc1 ; c1ccc(-c2nc3ccc4ccc5ccc(N(c6ccc7ccccc7c6)c6ccccc6[Si](c6ccccc6)(c6ccccc6)c6ccccc6)cc5c4c3o2)cc1 ; c1ccc(-c2ccc(N(c3ccc(-c4ccccc4)nc3)c3ccc4ccc5ccc6nc(-c7ccccc7)oc6c5c4c3)cn2)cc1]

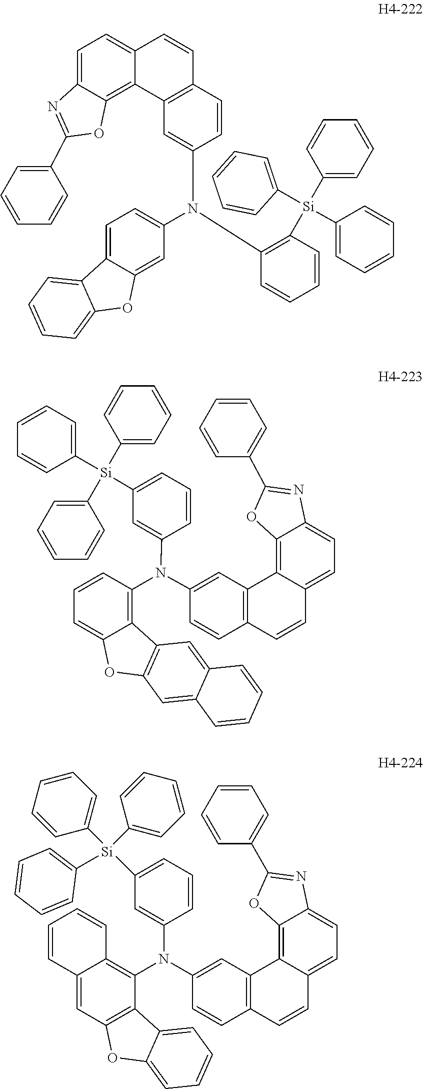 [SMILES: c1ccc(-c2nc3ccc4ccc5ccc(N(c6ccc7c(c6)oc6ccccc67)c6ccccc6[Si](c6ccccc6)(c6ccccc6)c6ccccc6)cc5c4c3o2)cc1 ; c1ccc(-c2nc3ccc4ccc5ccc(N(c6cccc([Si](c7ccccc7)(c7ccccc7)c7ccccc7)c6)c6cccc7oc8cc9ccccc9cc8c67)cc5c4c3o2)cc1 ; c1ccc(-c2nc3ccc4ccc5ccc(N(c6cccc([Si](c7ccccc7)(c7ccccc7)c7ccccc7)c6)c6c7ccccc7cc7oc8ccccc8c67)cc5c4c3o2)cc1]

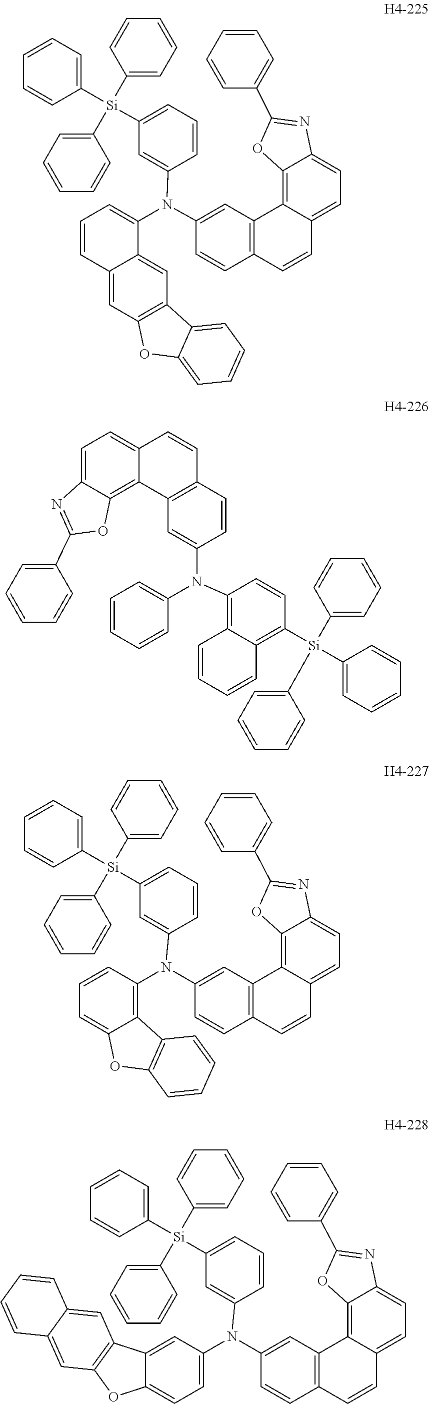

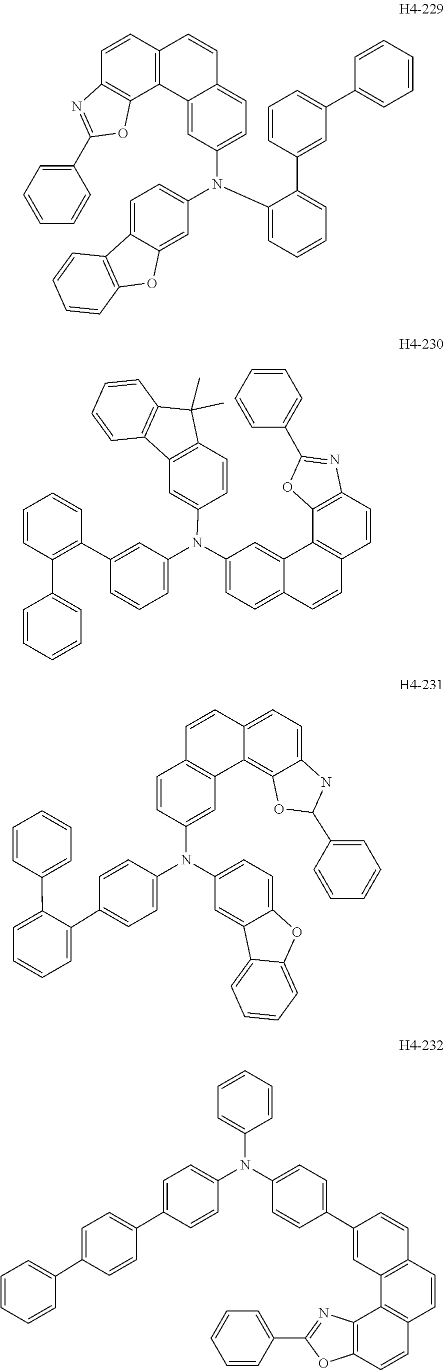

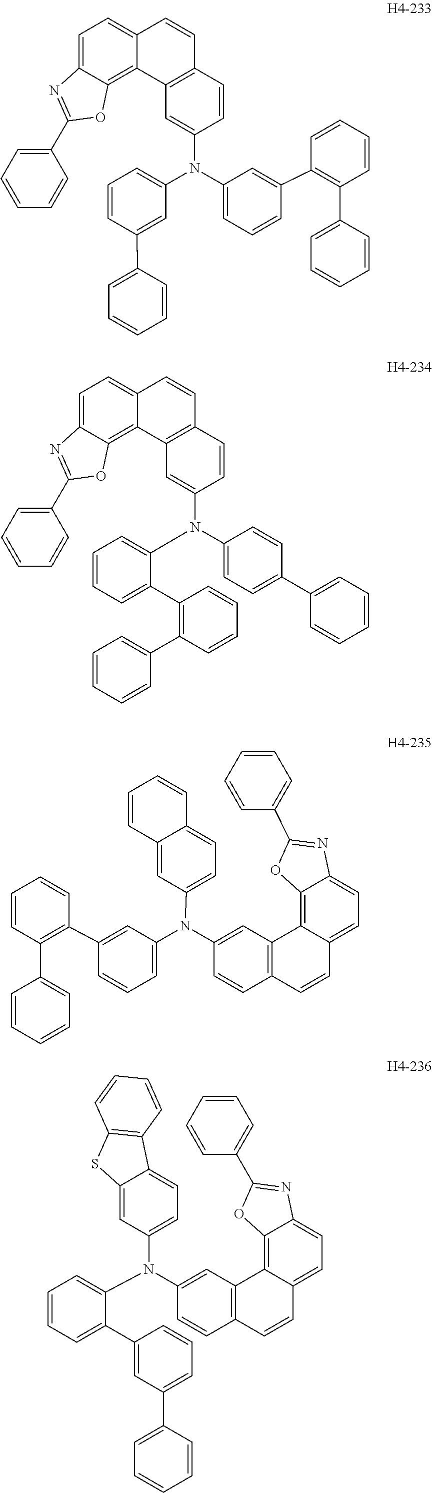 [SMILES: c1ccc(-c2cccc(N(c3cccc(-c4ccccc4-c4ccccc4)c3)c3ccc4ccc5ccc6nc(-c7ccccc7)oc6c5c4c3)c2)cc1 ; c1ccc(-c2ccc(N(c3ccc4ccc5ccc6nc(-c7ccccc7)oc6c5c4c3)c3ccccc3-c3ccccc3-c3ccccc3)cc2)cc1 ; c1ccc(-c2nc3ccc4ccc5ccc(N(c6cccc(-c7ccccc7-c7ccccc7)c6)c6ccc7ccccc7c6)cc5c4c3o2)cc1 ; c1ccc(-c2cccc(-c3ccccc3N(c3ccc4c(c3)sc3ccccc34)c3ccc4ccc5ccc6nc(-c7ccccc7)oc6c5c4c3)c2)cc1]

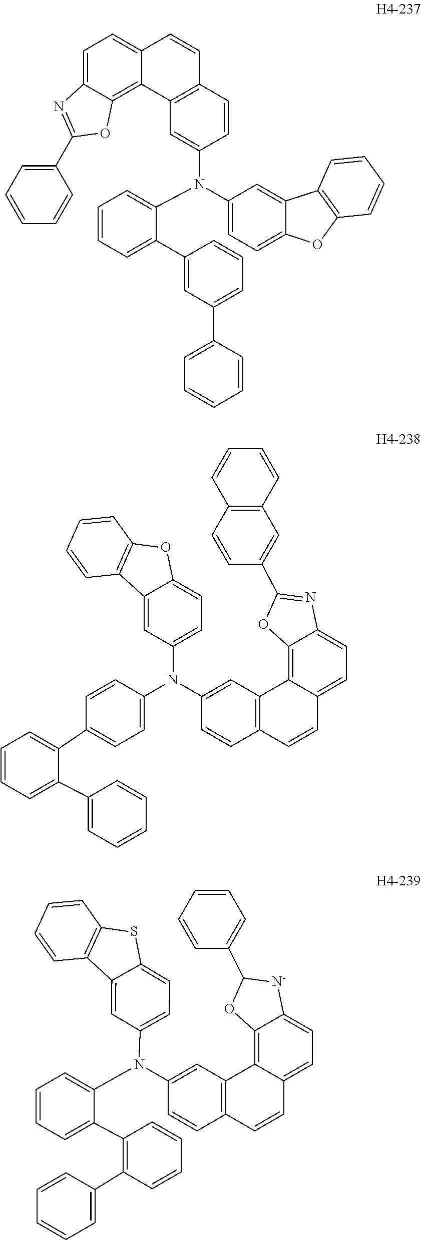

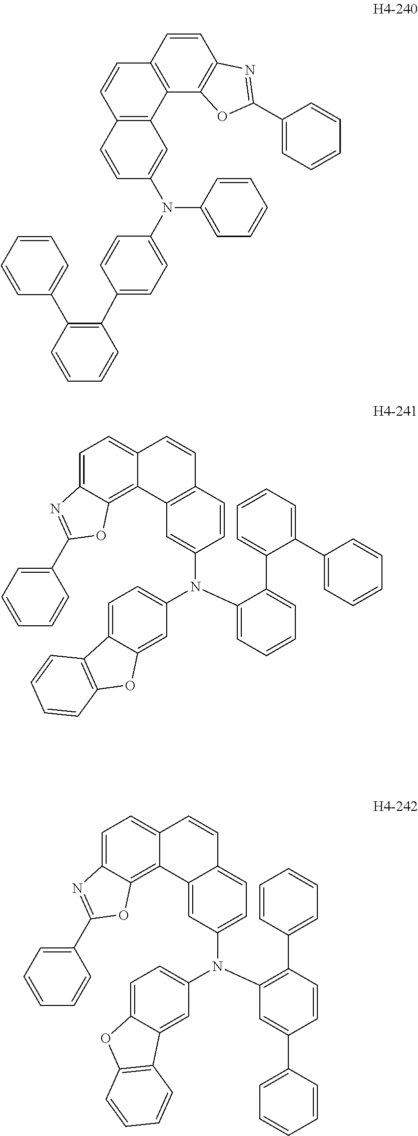 [SMILES: c1ccc(-c2nc3ccc4ccc5ccc(N(c6ccccc6)c6ccc(-c7ccccc7-c7ccccc7)cc6)cc5c4c3o2)cc1 ; c1ccc(-c2nc3ccc4ccc5ccc(N(c6ccc7c(c6)oc6ccccc67)c6ccccc6-c6ccccc6-c6ccccc6)cc5c4c3o2)cc1 ; c1ccc(-c2ccc(-c3ccccc3)c(N(c3ccc4oc5ccccc5c4c3)c3ccc4ccc5ccc6nc(-c7ccccc7)oc6c5c4c3)c2)cc1]

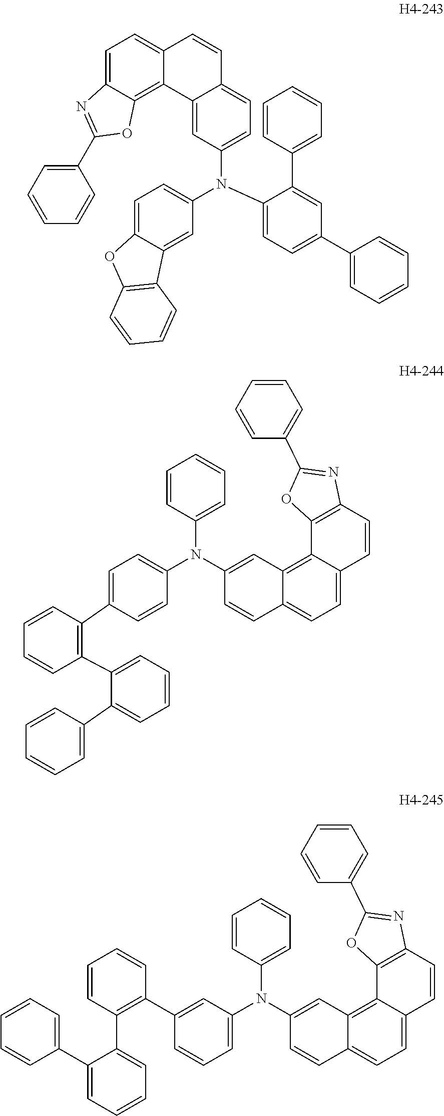 [SMILES: c1ccc(-c2ccc(N(c3ccc4oc5ccccc5c4c3)c3ccc4ccc5ccc6nc(-c7ccccc7)oc6c5c4c3)c(-c3ccccc3)c2)cc1 ; c1ccc(-c2nc3ccc4ccc5ccc(N(c6ccccc6)c6ccc(-c7ccccc7-c7ccccc7-c7ccccc7)cc6)cc5c4c3o2)cc1 ; c1ccc(-c2nc3ccc4ccc5ccc(N(c6ccccc6)c6cccc(-c7ccccc7-c7ccccc7-c7ccccc7)c6)cc5c4c3o2)cc1]

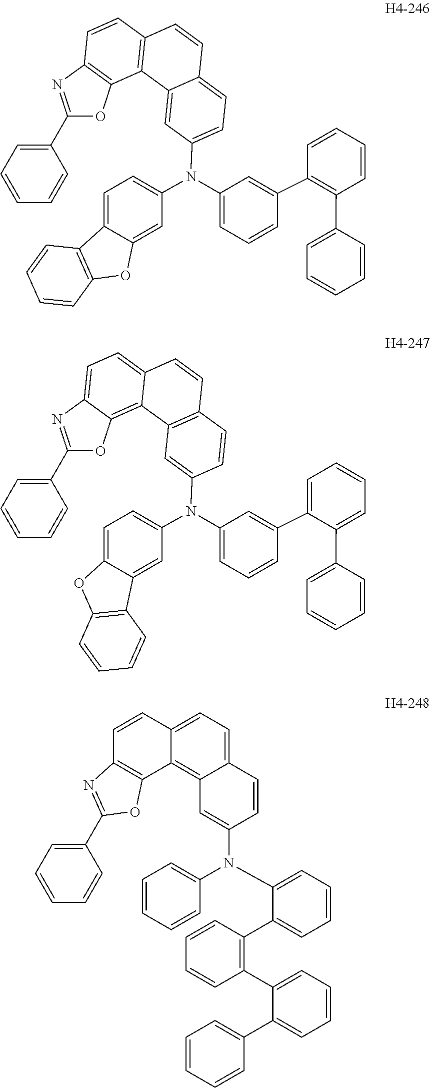

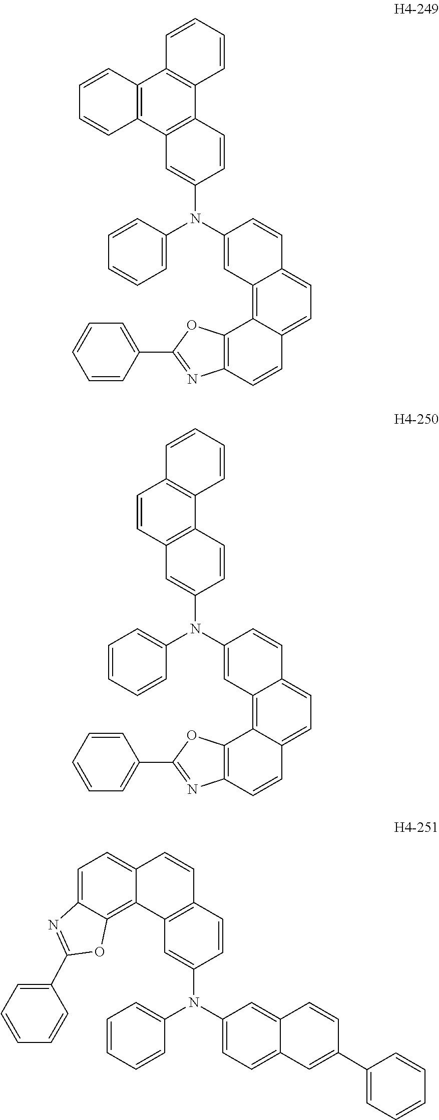 [SMILES: c1ccc(-c2nc3ccc4ccc5ccc(N(c6ccccc6)c6ccc7c8ccccc8c8ccccc8c7c6)cc5c4c3o2)cc1 ; c1ccc(-c2nc3ccc4ccc5ccc(N(c6ccccc6)c6ccc7c(ccc8ccccc87)c6)cc5c4c3o2)cc1 ; c1ccc(-c2ccc3cc(N(c4ccccc4)c4ccc5ccc6ccc7nc(-c8ccccc8)oc7c6c5c4)ccc3c2)cc1]

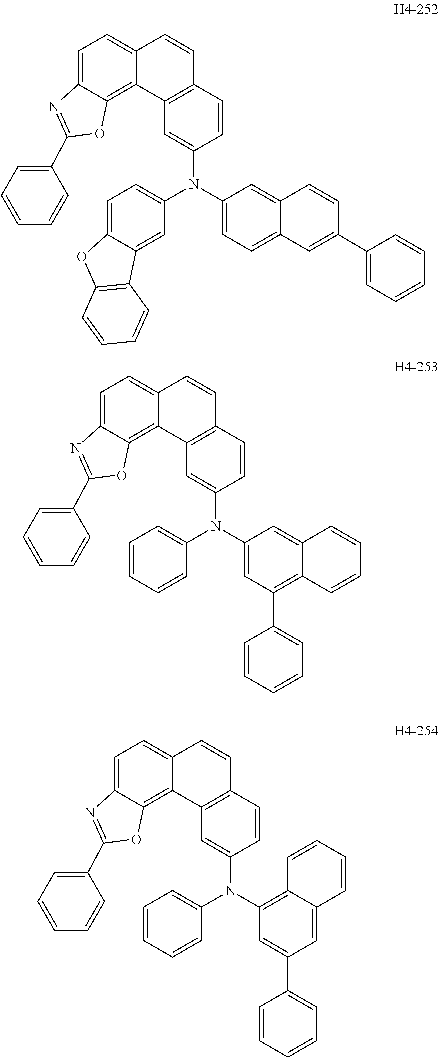

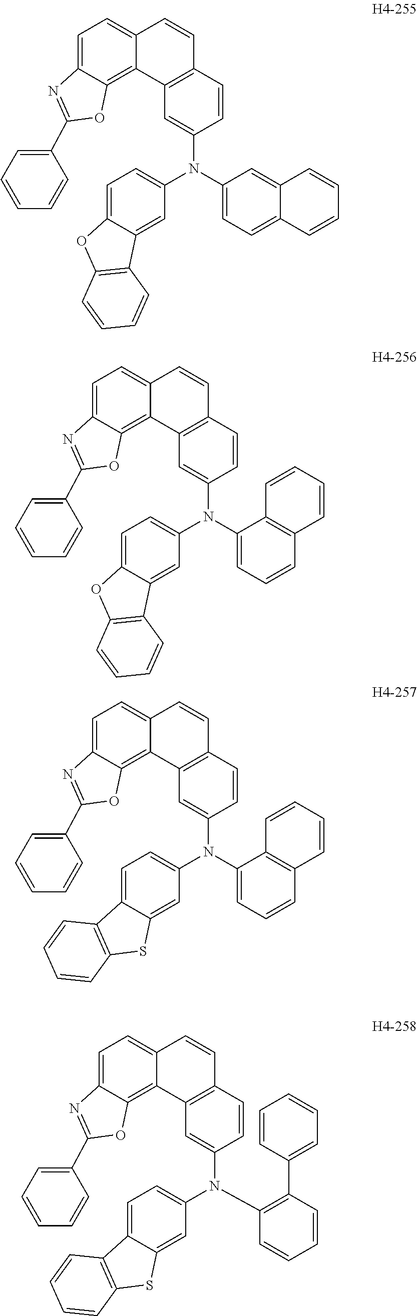

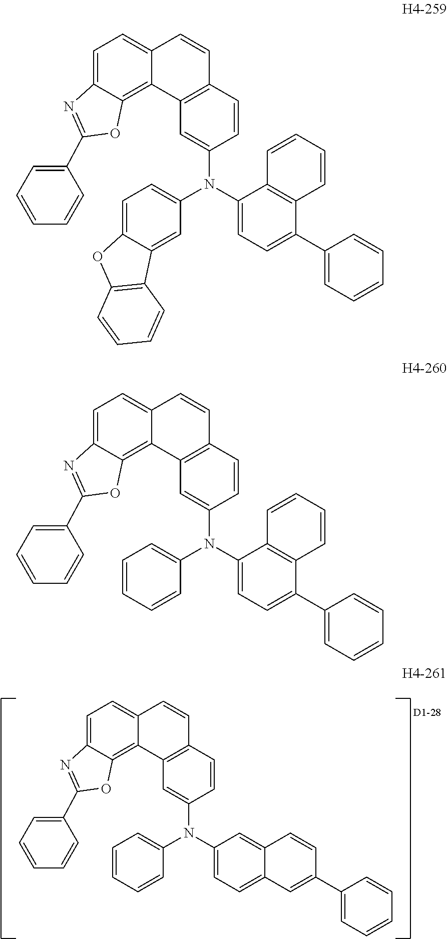

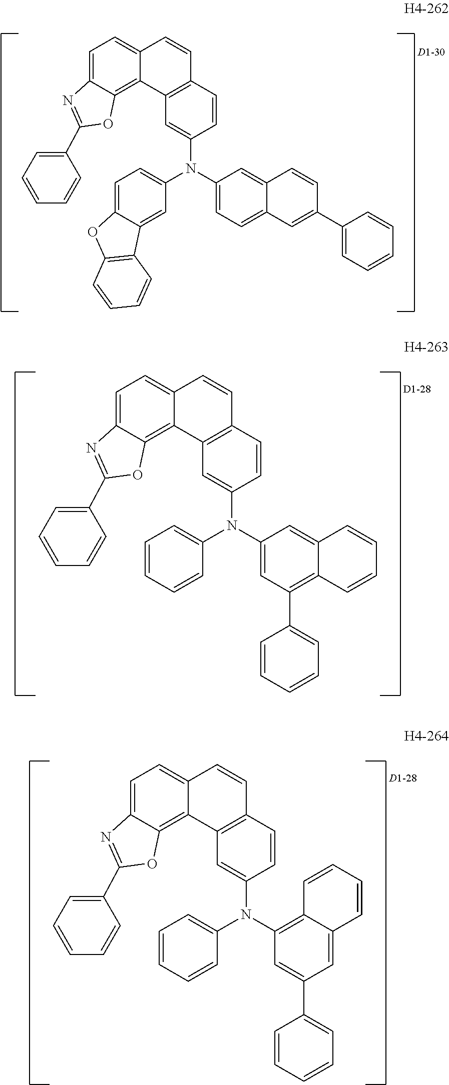

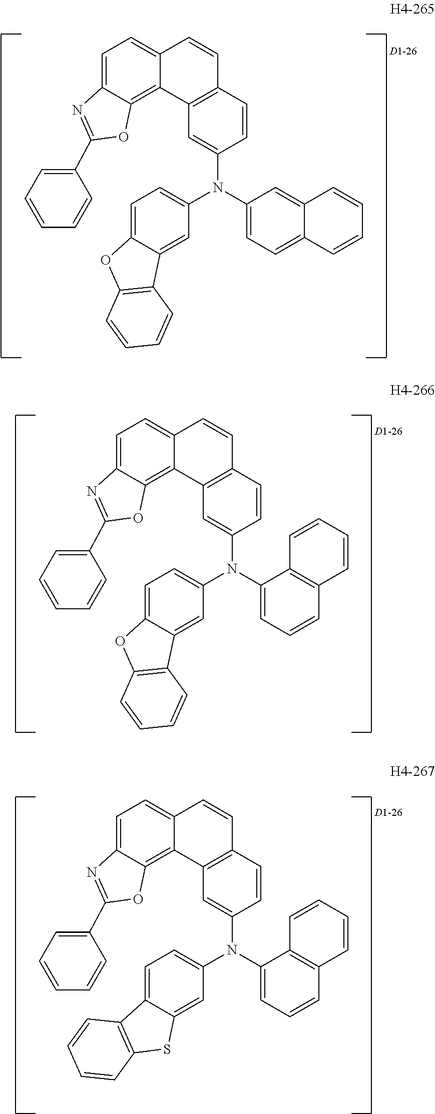

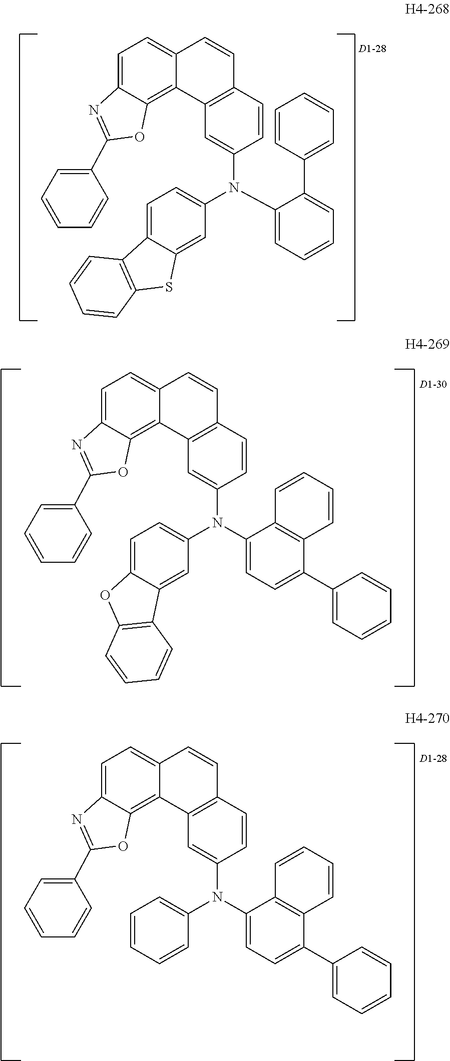

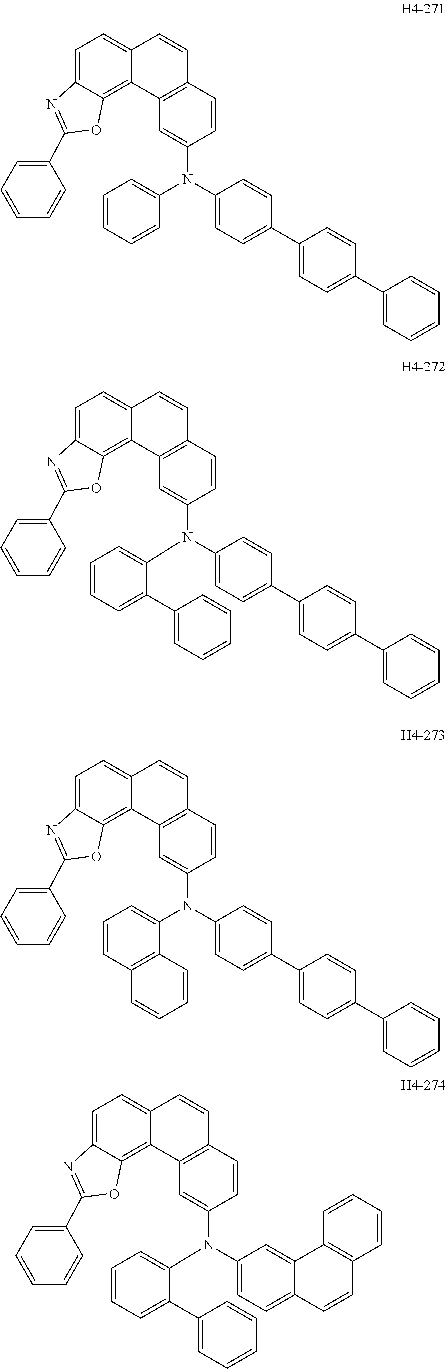 [SMILES: c1ccc(-c2ccc(-c3ccc(N(c4ccccc4)c4ccc5ccc6ccc7nc(-c8ccccc8)oc7c6c5c4)cc3)cc2)cc1 ; c1ccc(-c2ccc(-c3ccc(N(c4ccc5ccc6ccc7nc(-c8ccccc8)oc7c6c5c4)c4ccccc4-c4ccccc4)cc3)cc2)cc1 ; c1ccc(-c2ccc(-c3ccc(N(c4ccc5ccc6ccc7nc(-c8ccccc8)oc7c6c5c4)c4cccc5ccccc45)cc3)cc2)cc1 ; c1ccc(-c2nc3ccc4ccc5ccc(N(c6ccc7ccc8ccccc8c7c6)c6ccccc6-c6ccccc6)cc5c4c3o2)cc1]

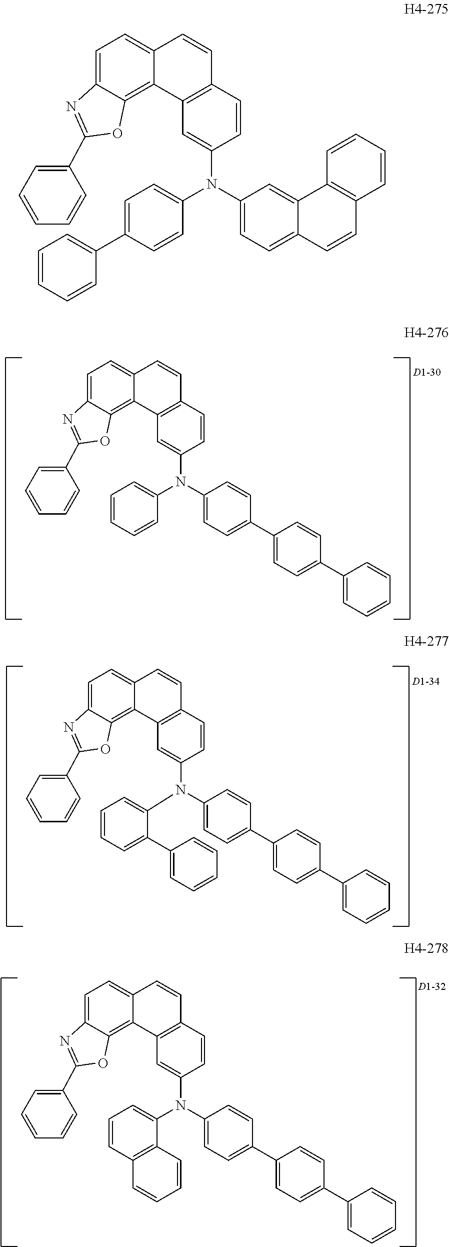

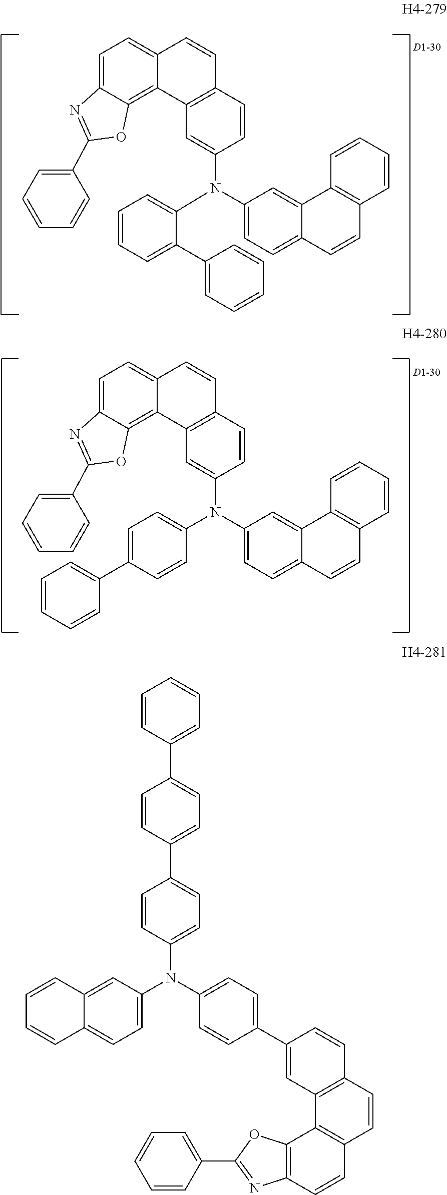

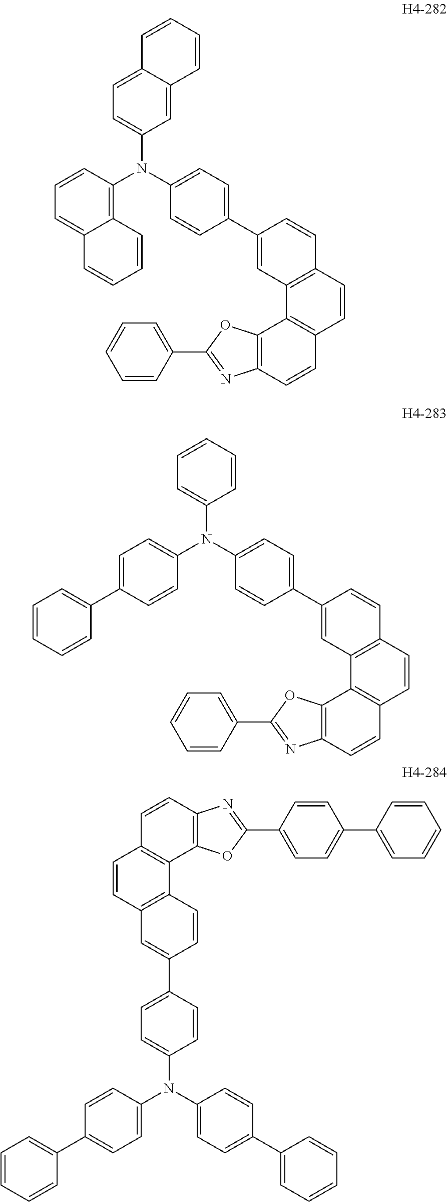 [SMILES: c1ccc(-c2nc3ccc4ccc5ccc(-c6ccc(N(c7ccc8ccccc8c7)c7cccc8ccccc78)cc6)cc5c4c3o2)cc1 ; c1ccc(-c2ccc(N(c3ccccc3)c3ccc(-c4ccc5ccc6ccc7nc(-c8ccccc8)oc7c6c5c4)cc3)cc2)cc1 ; c1ccc(-c2ccc(-c3nc4ccc5ccc6cc(-c7ccc(N(c8ccc(-c9ccccc9)cc8)c8ccc(-c9ccccc9)cc8)cc7)ccc6c5c4o3)cc2)cc1]

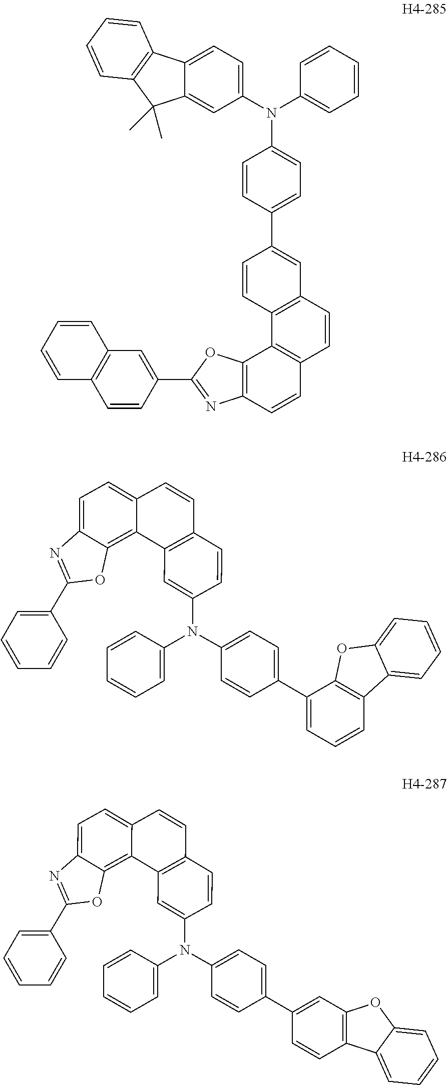 [SMILES: CC1(C)c2ccccc2-c2ccc(N(c3ccccc3)c3ccc(-c4ccc5c(ccc6ccc7nc(-c8ccc9ccccc9c8)oc7c65)c4)cc3)cc21 ; c1ccc(-c2nc3ccc4ccc5ccc(N(c6ccccc6)c6ccc(-c7cccc8c7oc7ccccc78)cc6)cc5c4c3o2)cc1 ; c1ccc(-c2nc3ccc4ccc5ccc(N(c6ccccc6)c6ccc(-c7ccc8c(c7)oc7ccccc78)cc6)cc5c4c3o2)cc1]

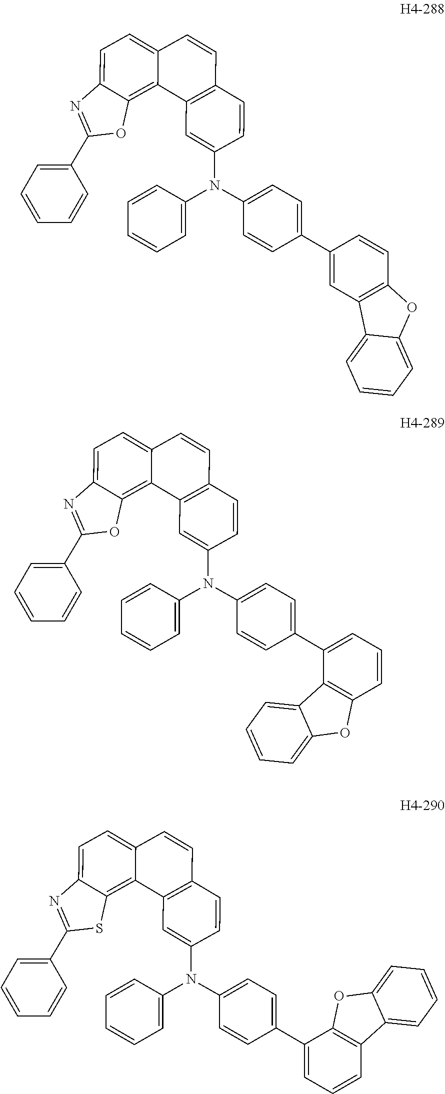 [SMILES: c1ccc(-c2nc3ccc4ccc5ccc(N(c6ccccc6)c6ccc(-c7ccc8oc9ccccc9c8c7)cc6)cc5c4c3o2)cc1 ; c1ccc(-c2nc3ccc4ccc5ccc(N(c6ccccc6)c6ccc(-c7cccc8oc9ccccc9c78)cc6)cc5c4c3o2)cc1 ; c1ccc(-c2nc3ccc4ccc5ccc(N(c6ccccc6)c6ccc(-c7cccc8c7oc7ccccc78)cc6)cc5c4c3s2)cc1]

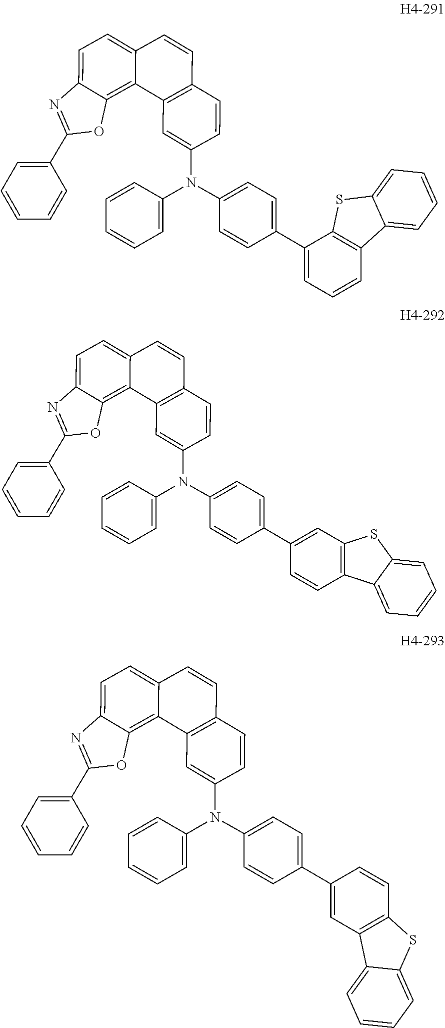 [SMILES: c1ccc(-c2nc3ccc4ccc5ccc(N(c6ccccc6)c6ccc(-c7cccc8c7sc7ccccc78)cc6)cc5c4c3o2)cc1 ; c1ccc(-c2nc3ccc4ccc5ccc(N(c6ccccc6)c6ccc(-c7ccc8c(c7)sc7ccccc78)cc6)cc5c4c3o2)cc1 ; c1ccc(-c2nc3ccc4ccc5ccc(N(c6ccccc6)c6ccc(-c7ccc8sc9ccccc9c8c7)cc6)cc5c4c3o2)cc1]

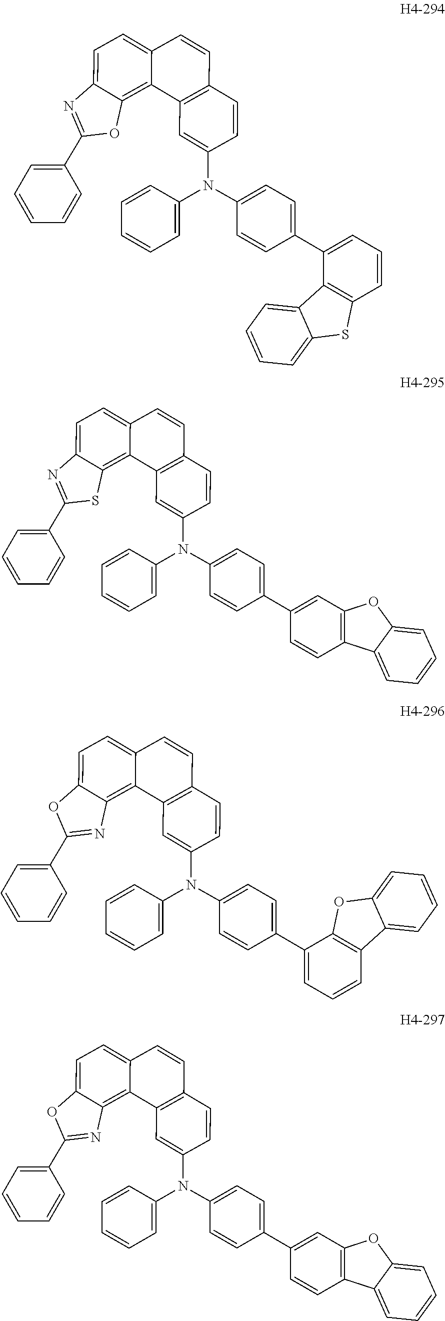 [SMILES: c1ccc(-c2nc3ccc4ccc5ccc(N(c6ccccc6)c6ccc(-c7cccc8sc9ccccc9c78)cc6)cc5c4c3o2)cc1 ; c1ccc(-c2nc3ccc4ccc5ccc(N(c6ccccc6)c6ccc(-c7ccc8c(c7)oc7ccccc78)cc6)cc5c4c3s2)cc1 ; c1ccc(-c2nc3c(ccc4ccc5ccc(N(c6ccccc6)c6ccc(-c7cccc8c7oc7ccccc78)cc6)cc5c43)o2)cc1 ; c1ccc(-c2nc3c(ccc4ccc5ccc(N(c6ccccc6)c6ccc(-c7ccc8c(c7)oc7ccccc78)cc6)cc5c43)o2)cc1]

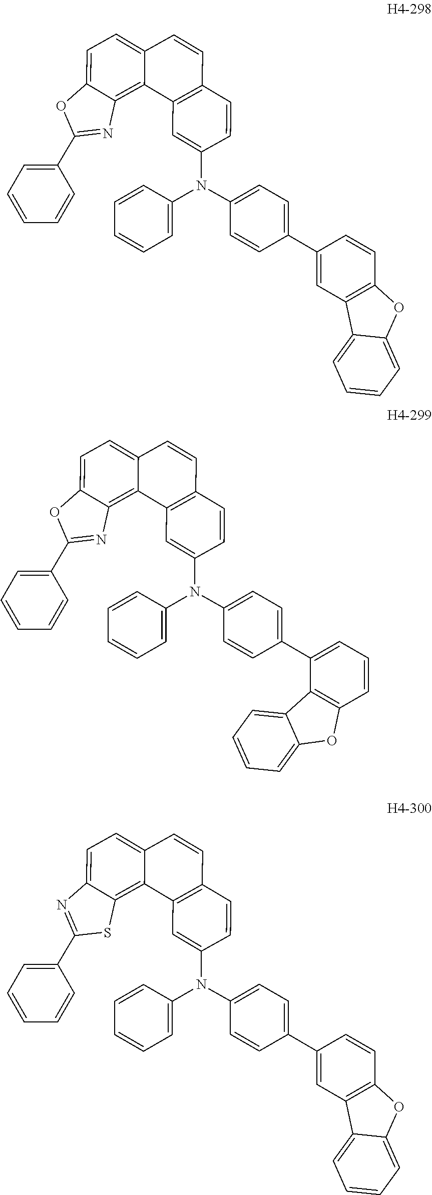 [SMILES: c1ccc(-c2nc3c(ccc4ccc5ccc(N(c6ccccc6)c6ccc(-c7ccc8oc9ccccc9c8c7)cc6)cc5c43)o2)cc1 ; c1ccc(-c2nc3c(ccc4ccc5ccc(N(c6ccccc6)c6ccc(-c7cccc8oc9ccccc9c78)cc6)cc5c43)o2)cc1 ; c1ccc(-c2nc3ccc4ccc5ccc(N(c6ccccc6)c6ccc(-c7ccc8oc9ccccc9c8c7)cc6)cc5c4c3s2)cc1]

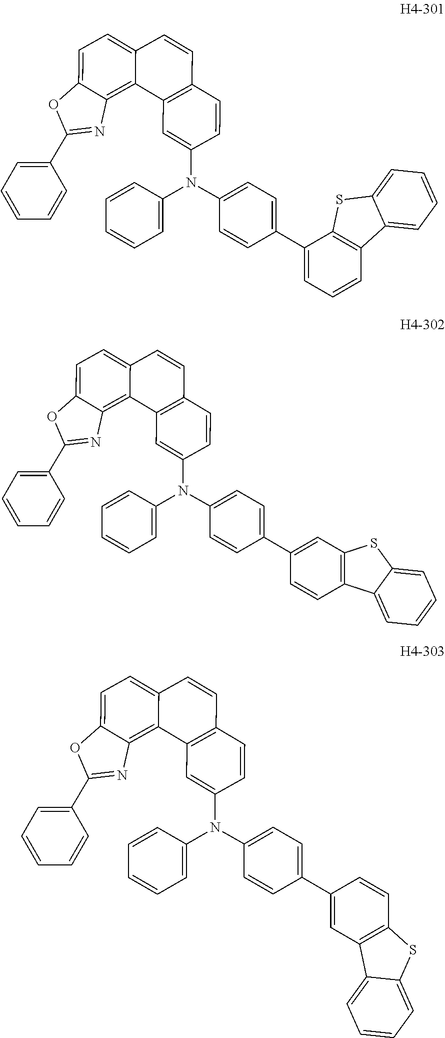 [SMILES: c1ccc(-c2nc3c(ccc4ccc5ccc(N(c6ccccc6)c6ccc(-c7cccc8c7sc7ccccc78)cc6)cc5c43)o2)cc1 ; c1ccc(-c2nc3c(ccc4ccc5ccc(N(c6ccccc6)c6ccc(-c7ccc8c(c7)sc7ccccc78)cc6)cc5c43)o2)cc1 ; c1ccc(-c2nc3c(ccc4ccc5ccc(N(c6ccccc6)c6ccc(-c7ccc8sc9ccccc9c8c7)cc6)cc5c43)o2)cc1]

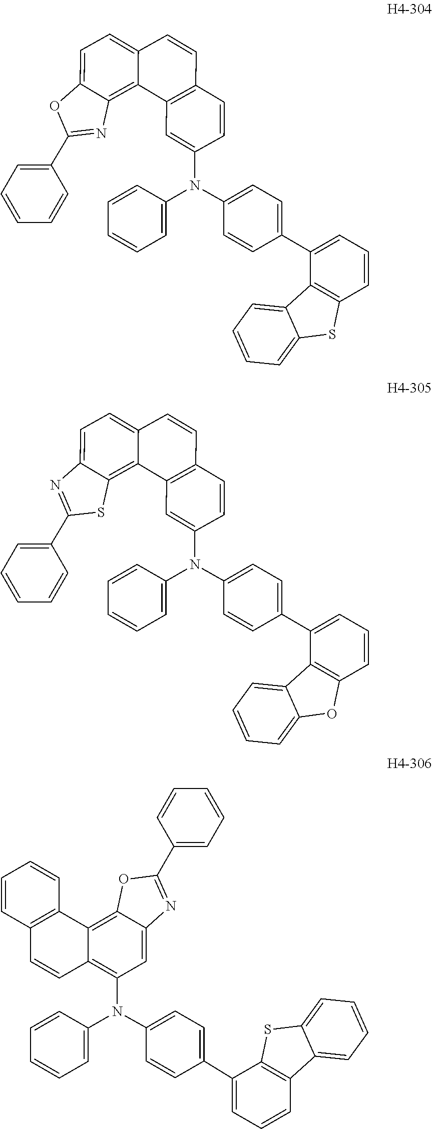 [SMILES: c1ccc(-c2nc3c(ccc4ccc5ccc(N(c6ccccc6)c6ccc(-c7cccc8sc9ccccc9c78)cc6)cc5c43)o2)cc1 ; c1ccc(-c2nc3ccc4ccc5ccc(N(c6ccccc6)c6ccc(-c7cccc8oc9ccccc9c78)cc6)cc5c4c3s2)cc1 ; c1ccc(-c2nc3cc(N(c4ccccc4)c4ccc(-c5cccc6c5sc5ccccc56)cc4)c4ccc5ccccc5c4c3o2)cc1]

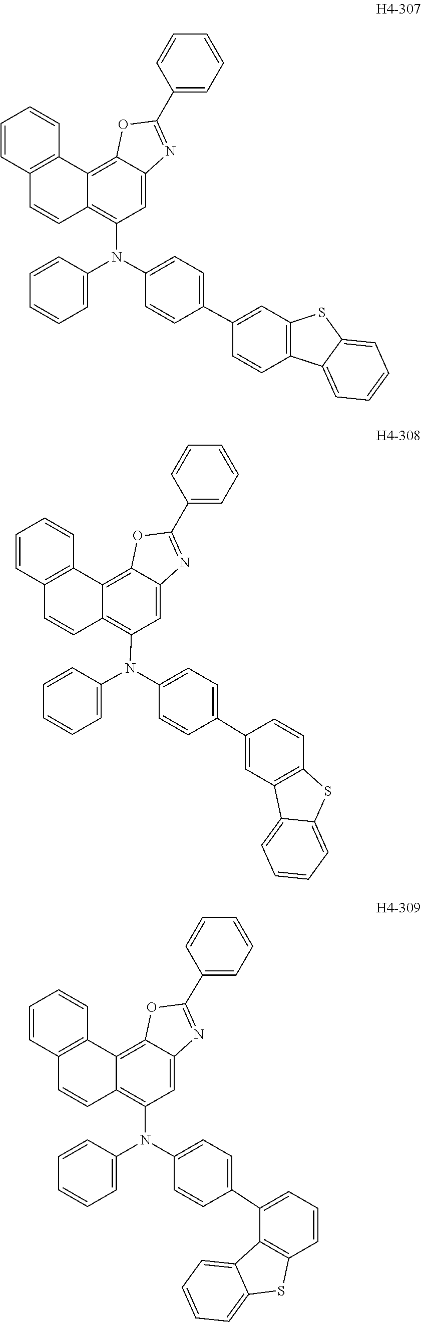 [SMILES: c1ccc(-c2nc3cc(N(c4ccccc4)c4ccc(-c5ccc6c(c5)sc5ccccc56)cc4)c4ccc5ccccc5c4c3o2)cc1 ; c1ccc(-c2nc3cc(N(c4ccccc4)c4ccc(-c5ccc6sc7ccccc7c6c5)cc4)c4ccc5ccccc5c4c3o2)cc1 ; c1ccc(-c2nc3cc(N(c4ccccc4)c4ccc(-c5cccc6sc7ccccc7c56)cc4)c4ccc5ccccc5c4c3o2)cc1]

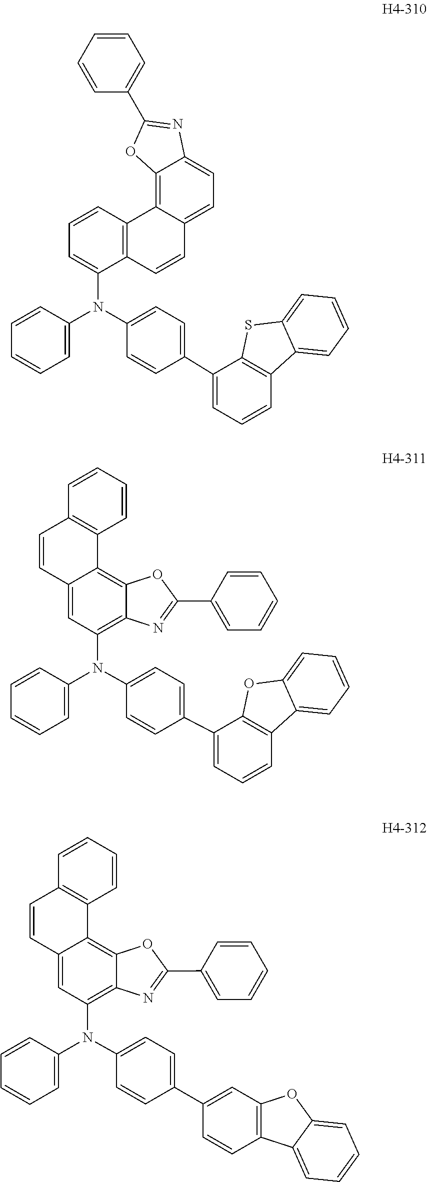 [SMILES: c1ccc(-c2nc3ccc4ccc5c(N(c6ccccc6)c6ccc(-c7cccc8c7sc7ccccc78)cc6)cccc5c4c3o2)cc1 ; c1ccc(-c2nc3c(N(c4ccccc4)c4ccc(-c5cccc6c5oc5ccccc56)cc4)cc4ccc5ccccc5c4c3o2)cc1 ; c1ccc(-c2nc3c(N(c4ccccc4)c4ccc(-c5ccc6c(c5)oc5ccccc56)cc4)cc4ccc5ccccc5c4c3o2)cc1]

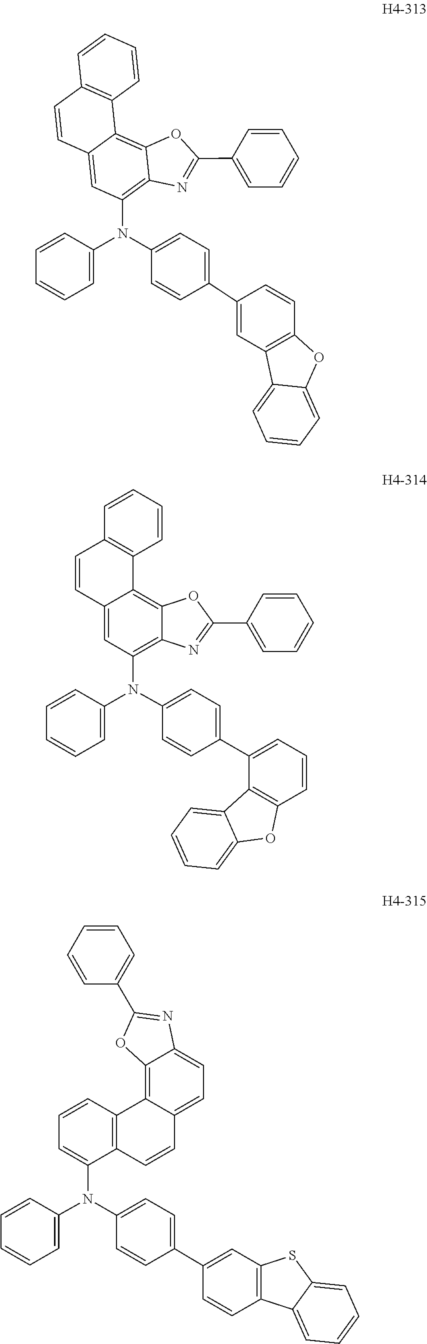 [SMILES: c1ccc(-c2nc3c(N(c4ccccc4)c4ccc(-c5ccc6oc7ccccc7c6c5)cc4)cc4ccc5ccccc5c4c3o2)cc1 ; c1ccc(-c2nc3c(N(c4ccccc4)c4ccc(-c5cccc6oc7ccccc7c56)cc4)cc4ccc5ccccc5c4c3o2)cc1 ; c1ccc(-c2nc3ccc4ccc5c(N(c6ccccc6)c6ccc(-c7ccc8c(c7)sc7ccccc78)cc6)cccc5c4c3o2)cc1]

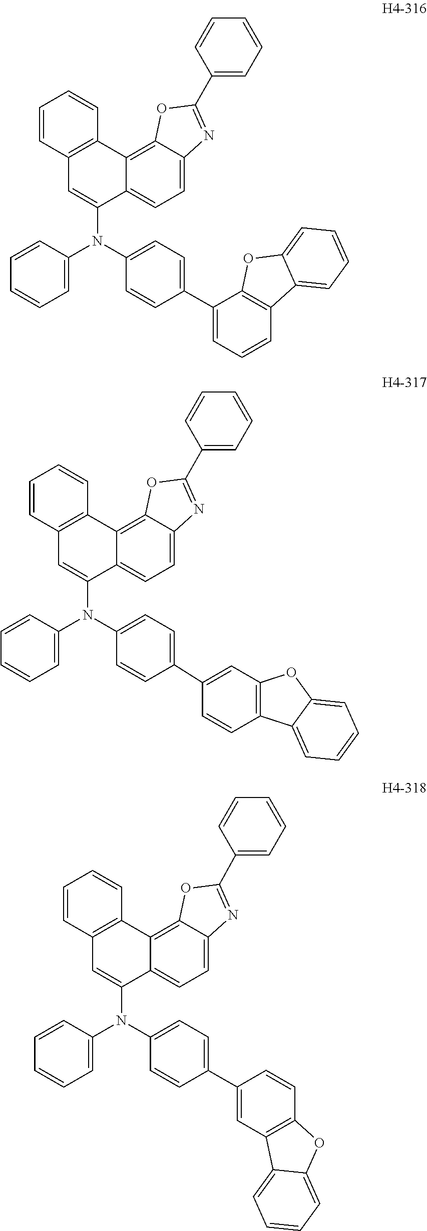

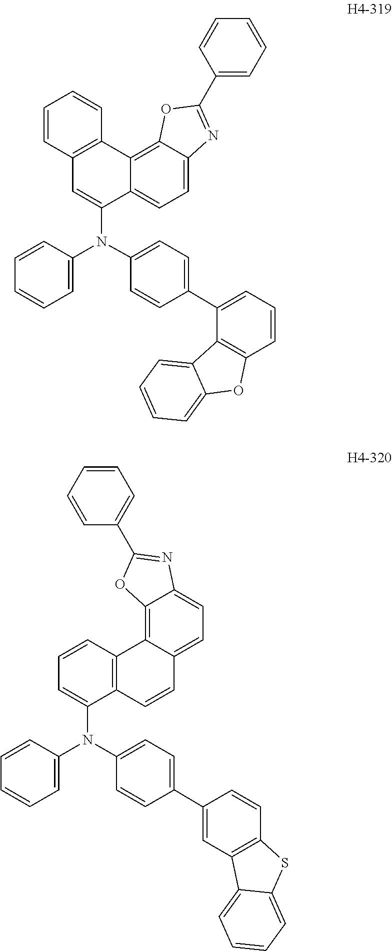 [SMILES: c1ccc(-c2nc3ccc4c(N(c5ccccc5)c5ccc(-c6cccc7oc8ccccc8c67)cc5)cc5ccccc5c4c3o2)cc1 ; c1ccc(-c2nc3ccc4ccc5c(N(c6ccccc6)c6ccc(-c7ccc8sc9ccccc9c8c7)cc6)cccc5c4c3o2)cc1]

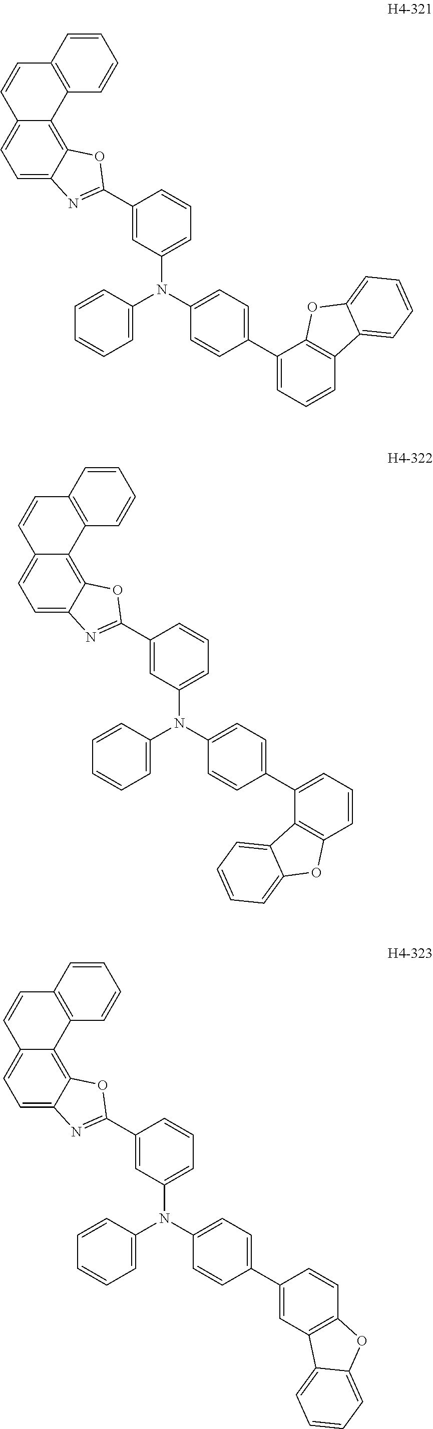 [SMILES: c1ccc(N(c2ccc(-c3cccc4c3oc3ccccc34)cc2)c2cccc(-c3nc4ccc5ccc6ccccc6c5c4o3)c2)cc1 ; c1ccc(N(c2ccc(-c3cccc4oc5ccccc5c34)cc2)c2cccc(-c3nc4ccc5ccc6ccccc6c5c4o3)c2)cc1 ; c1ccc(N(c2ccc(-c3ccc4oc5ccccc5c4c3)cc2)c2cccc(-c3nc4ccc5ccc6ccccc6c5c4o3)c2)cc1]

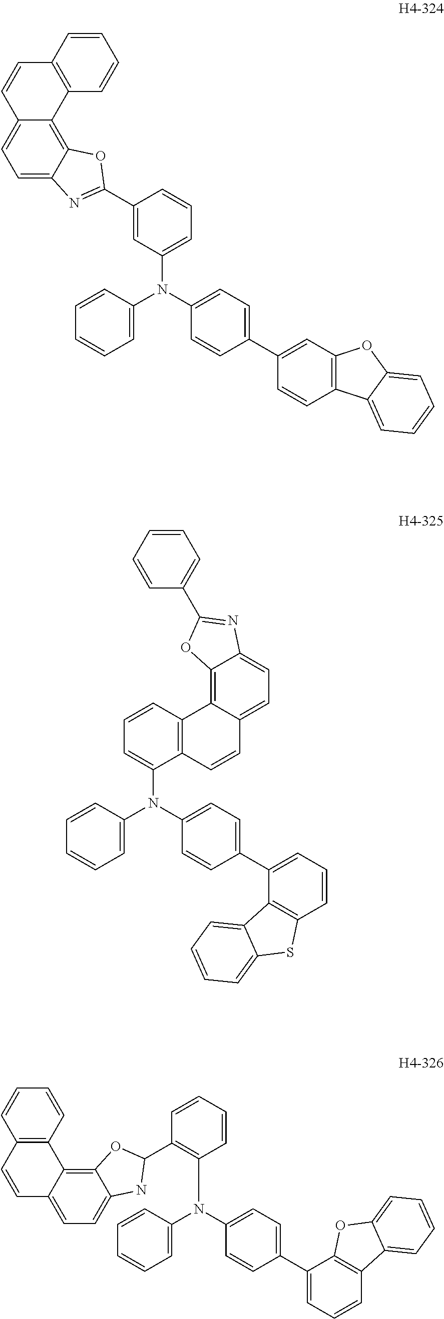

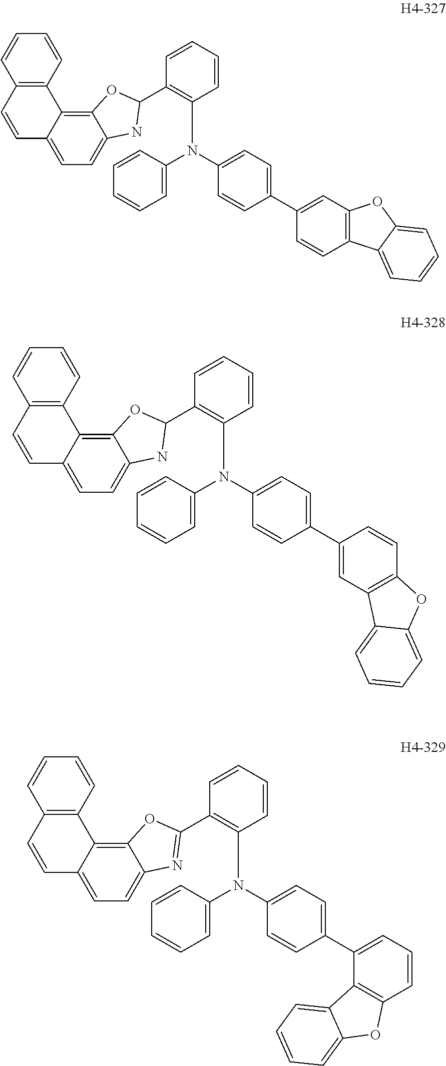

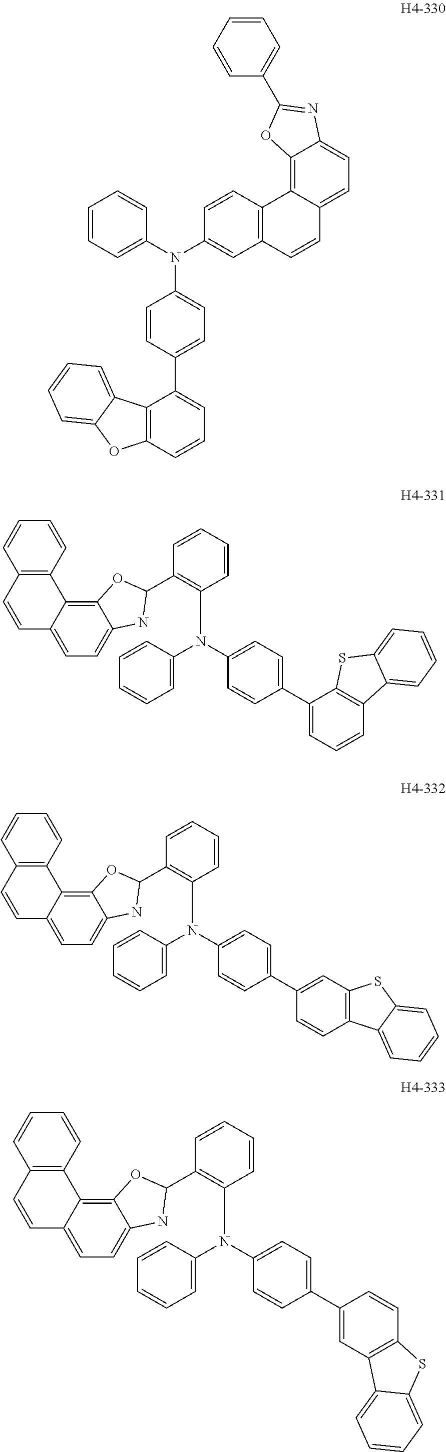

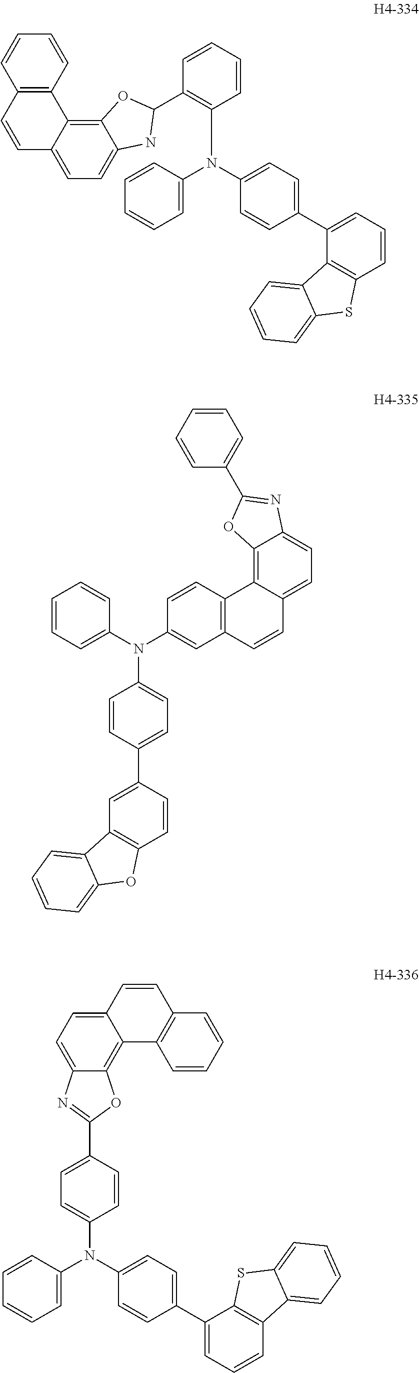

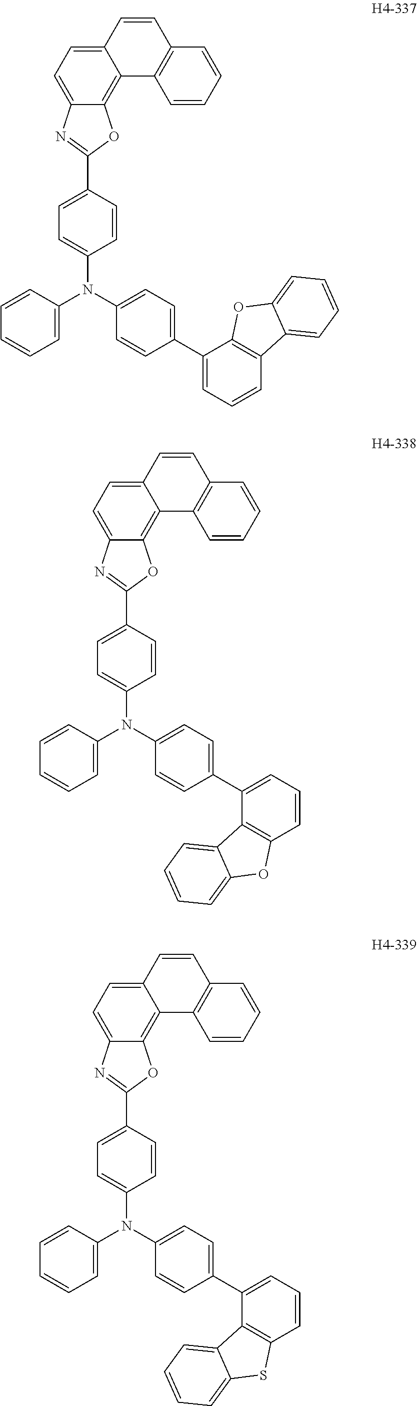 [SMILES: c1ccc(N(c2ccc(-c3nc4ccc5ccc6ccccc6c5c4o3)cc2)c2ccc(-c3cccc4c3oc3ccccc34)cc2)cc1 ; c1ccc(N(c2ccc(-c3nc4ccc5ccc6ccccc6c5c4o3)cc2)c2ccc(-c3cccc4oc5ccccc5c34)cc2)cc1 ; c1ccc(N(c2ccc(-c3nc4ccc5ccc6ccccc6c5c4o3)cc2)c2ccc(-c3cccc4sc5ccccc5c34)cc2)cc1]

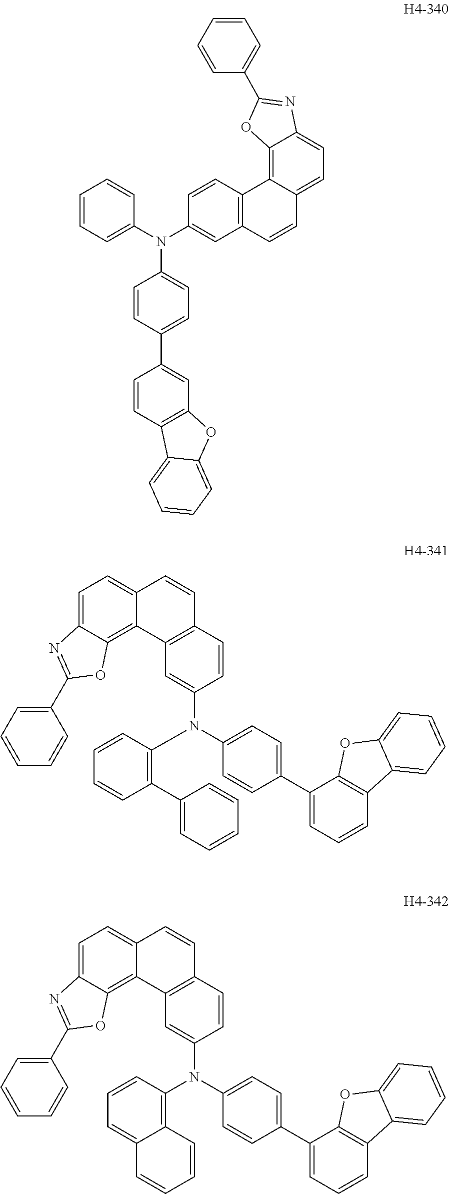 [SMILES: c1ccc(-c2nc3ccc4ccc5cc(N(c6ccccc6)c6ccc(-c7ccc8c(c7)oc7ccccc78)cc6)ccc5c4c3o2)cc1 ; c1ccc(-c2nc3ccc4ccc5ccc(N(c6ccc(-c7cccc8c7oc7ccccc78)cc6)c6ccccc6-c6ccccc6)cc5c4c3o2)cc1 ; c1ccc(-c2nc3ccc4ccc5ccc(N(c6ccc(-c7cccc8c7oc7ccccc78)cc6)c6cccc7ccccc67)cc5c4c3o2)cc1]

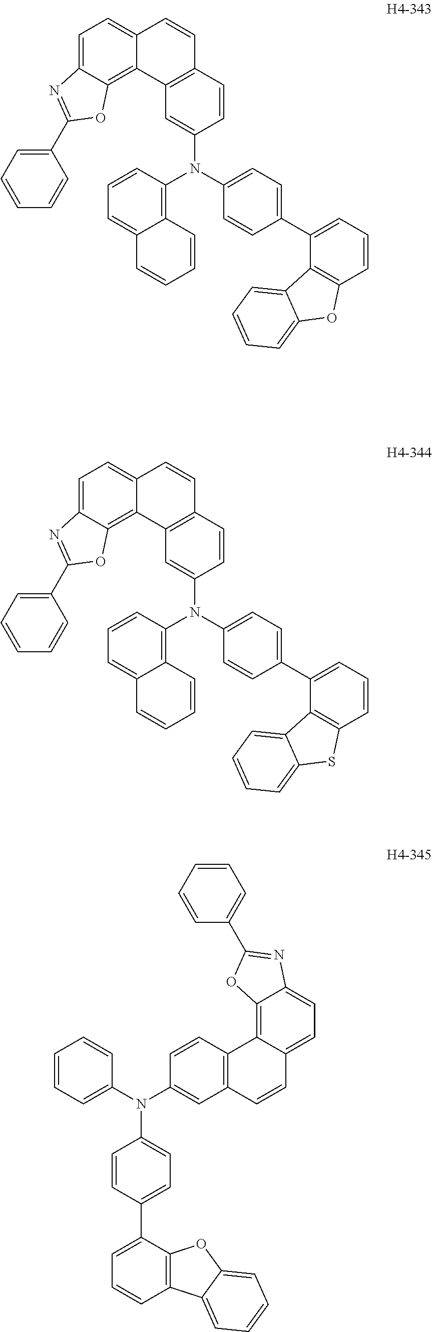 [SMILES: c1ccc(-c2nc3ccc4ccc5ccc(N(c6ccc(-c7cccc8oc9ccccc9c78)cc6)c6cccc7ccccc67)cc5c4c3o2)cc1 ; c1ccc(-c2nc3ccc4ccc5ccc(N(c6ccc(-c7cccc8sc9ccccc9c78)cc6)c6cccc7ccccc67)cc5c4c3o2)cc1 ; c1ccc(-c2nc3ccc4ccc5cc(N(c6ccccc6)c6ccc(-c7cccc8c7oc7ccccc78)cc6)ccc5c4c3o2)cc1]

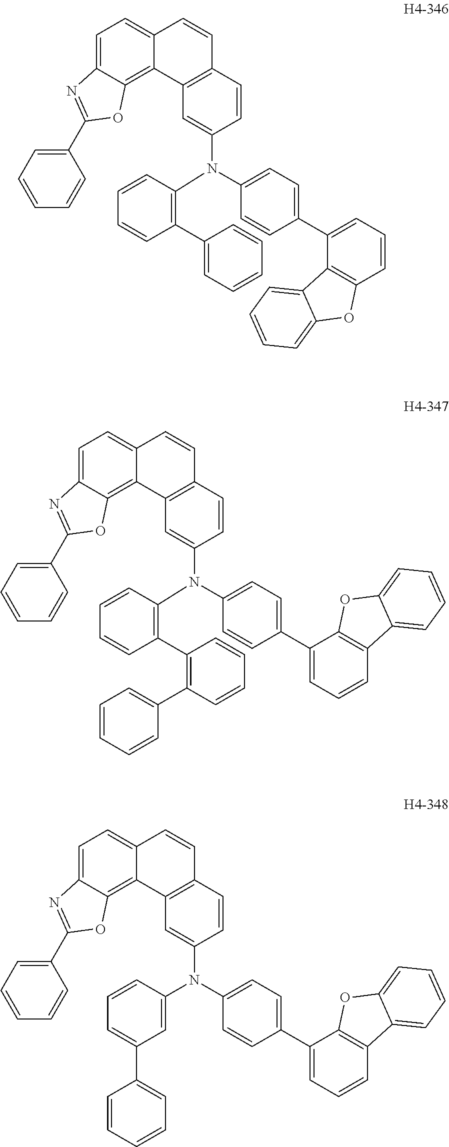 [SMILES: c1ccc(-c2nc3ccc4ccc5ccc(N(c6ccc(-c7cccc8oc9ccccc9c78)cc6)c6ccccc6-c6ccccc6)cc5c4c3o2)cc1 ; c1ccc(-c2nc3ccc4ccc5ccc(N(c6ccc(-c7cccc8c7oc7ccccc78)cc6)c6ccccc6-c6ccccc6-c6ccccc6)cc5c4c3o2)cc1 ; c1ccc(-c2cccc(N(c3ccc(-c4cccc5c4oc4ccccc45)cc3)c3ccc4ccc5ccc6nc(-c7ccccc7)oc6c5c4c3)c2)cc1]

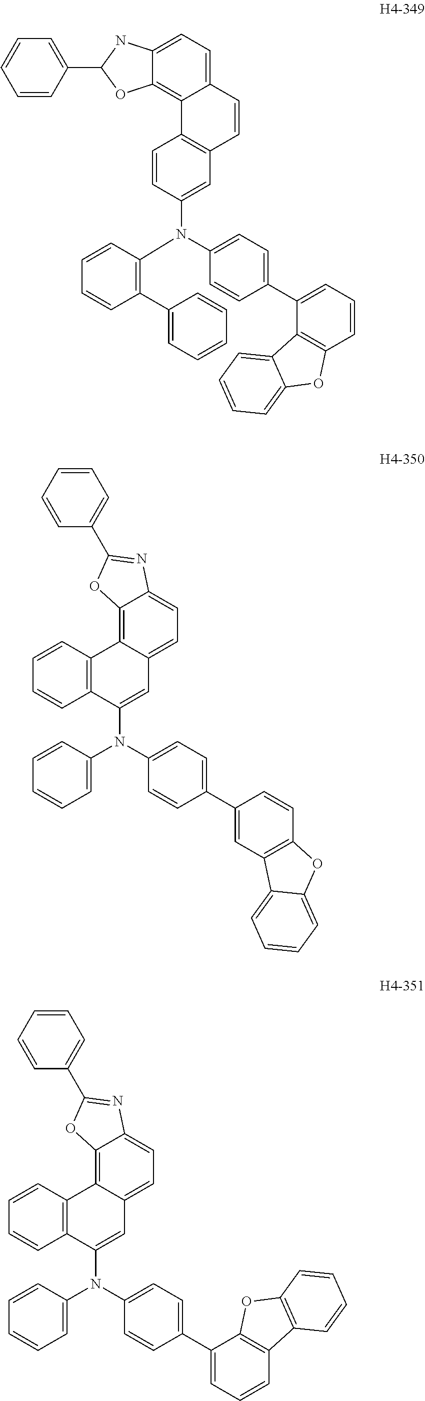

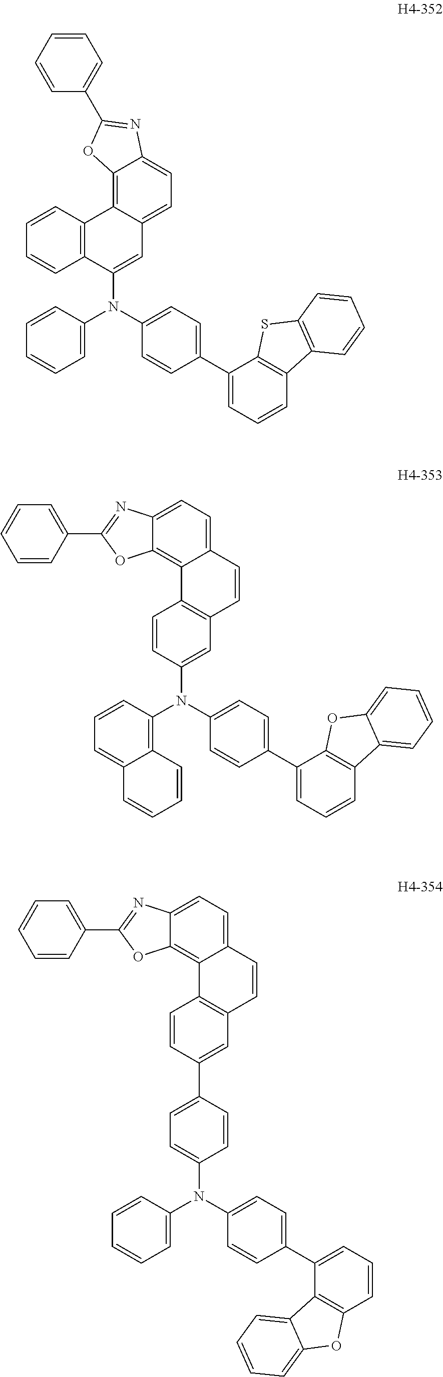 [SMILES: c1ccc(-c2nc3ccc4cc(N(c5ccccc5)c5ccc(-c6cccc7c6sc6ccccc67)cc5)c5ccccc5c4c3o2)cc1 ; c1ccc(-c2nc3ccc4ccc5cc(N(c6ccc(-c7cccc8c7oc7ccccc78)cc6)c6cccc7ccccc67)ccc5c4c3o2)cc1 ; c1ccc(-c2nc3ccc4ccc5cc(-c6ccc(N(c7ccccc7)c7ccc(-c8cccc9oc%10ccccc%10c89)cc7)cc6)ccc5c4c3o2)cc1]

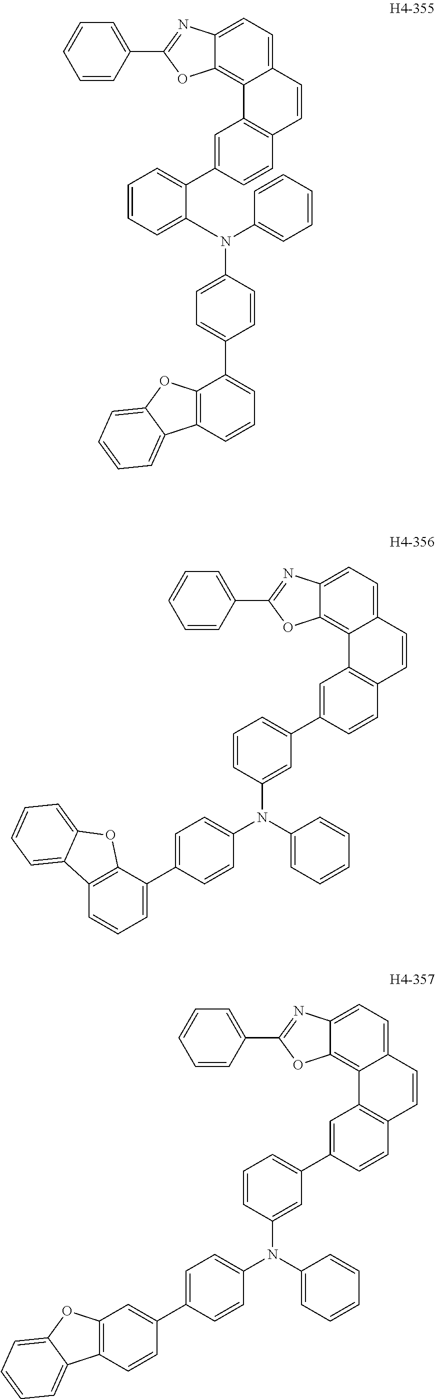 [SMILES: c1ccc(-c2nc3ccc4ccc5ccc(-c6ccccc6N(c6ccccc6)c6ccc(-c7cccc8c7oc7ccccc78)cc6)cc5c4c3o2)cc1 ; c1ccc(-c2nc3ccc4ccc5ccc(-c6cccc(N(c7ccccc7)c7ccc(-c8cccc9c8oc8ccccc89)cc7)c6)cc5c4c3o2)cc1 ; c1ccc(-c2nc3ccc4ccc5ccc(-c6cccc(N(c7ccccc7)c7ccc(-c8ccc9c(c8)oc8ccccc89)cc7)c6)cc5c4c3o2)cc1]

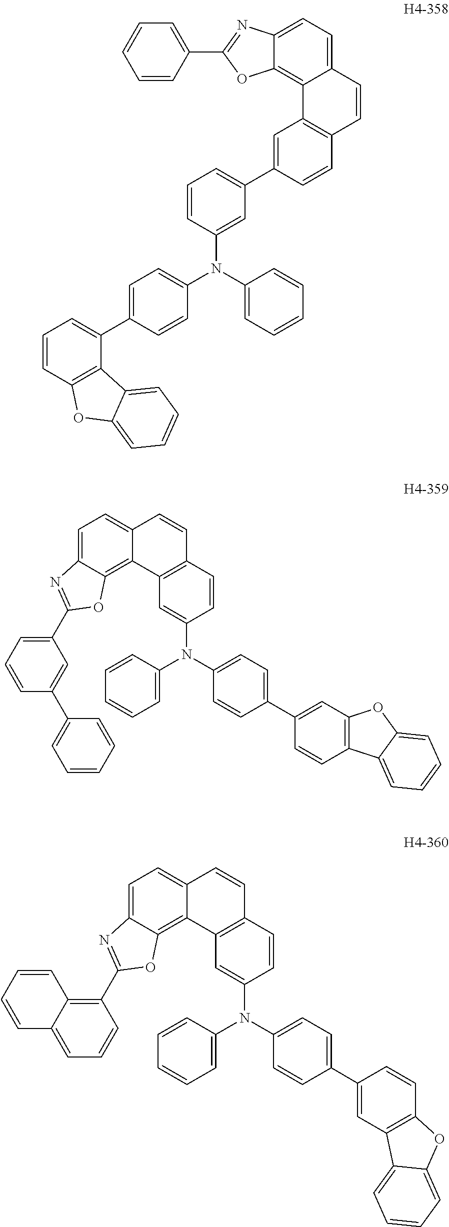 [SMILES: c1ccc(-c2nc3ccc4ccc5ccc(-c6cccc(N(c7ccccc7)c7ccc(-c8cccc9oc%10ccccc%10c89)cc7)c6)cc5c4c3o2)cc1 ; c1ccc(-c2cccc(-c3nc4ccc5ccc6ccc(N(c7ccccc7)c7ccc(-c8ccc9c(c8)oc8ccccc89)cc7)cc6c5c4o3)c2)cc1 ; c1ccc(N(c2ccc(-c3ccc4oc5ccccc5c4c3)cc2)c2ccc3ccc4ccc5nc(-c6cccc7ccccc67)oc5c4c3c2)cc1]

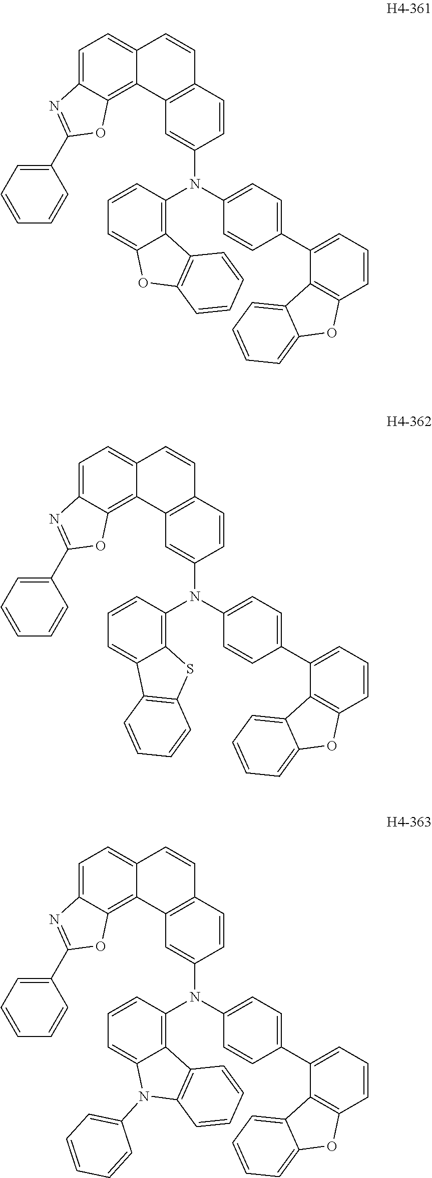 [SMILES: c1ccc(-c2nc3ccc4ccc5ccc(N(c6ccc(-c7cccc8oc9ccccc9c78)cc6)c6cccc7oc8ccccc8c67)cc5c4c3o2)cc1 ; c1ccc(-c2nc3ccc4ccc5ccc(N(c6ccc(-c7cccc8oc9ccccc9c78)cc6)c6cccc7c6sc6ccccc67)cc5c4c3o2)cc1 ; c1ccc(-c2nc3ccc4ccc5ccc(N(c6ccc(-c7cccc8oc9ccccc9c78)cc6)c6cccc7c6c6ccccc6n7-c6ccccc6)cc5c4c3o2)cc1]

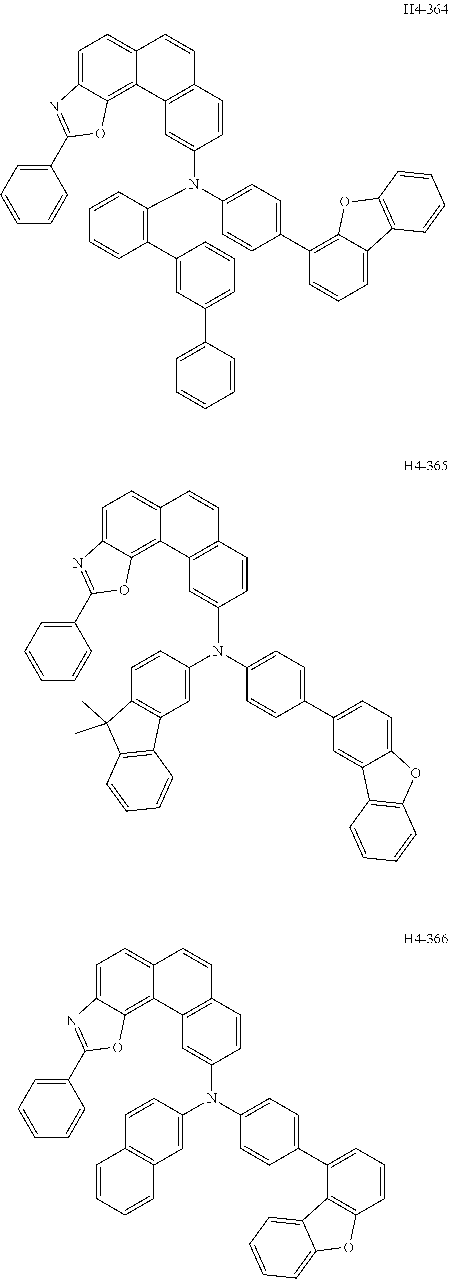 [SMILES: c1ccc(-c2cccc(-c3ccccc3N(c3ccc(-c4cccc5c4oc4ccccc45)cc3)c3ccc4ccc5ccc6nc(-c7ccccc7)oc6c5c4c3)c2)cc1 ; CC1(C)c2ccccc2-c2cc(N(c3ccc(-c4ccc5oc6ccccc6c5c4)cc3)c3ccc4ccc5ccc6nc(-c7ccccc7)oc6c5c4c3)ccc21 ; c1ccc(-c2nc3ccc4ccc5ccc(N(c6ccc(-c7cccc8oc9ccccc9c78)cc6)c6ccc7ccccc7c6)cc5c4c3o2)cc1]

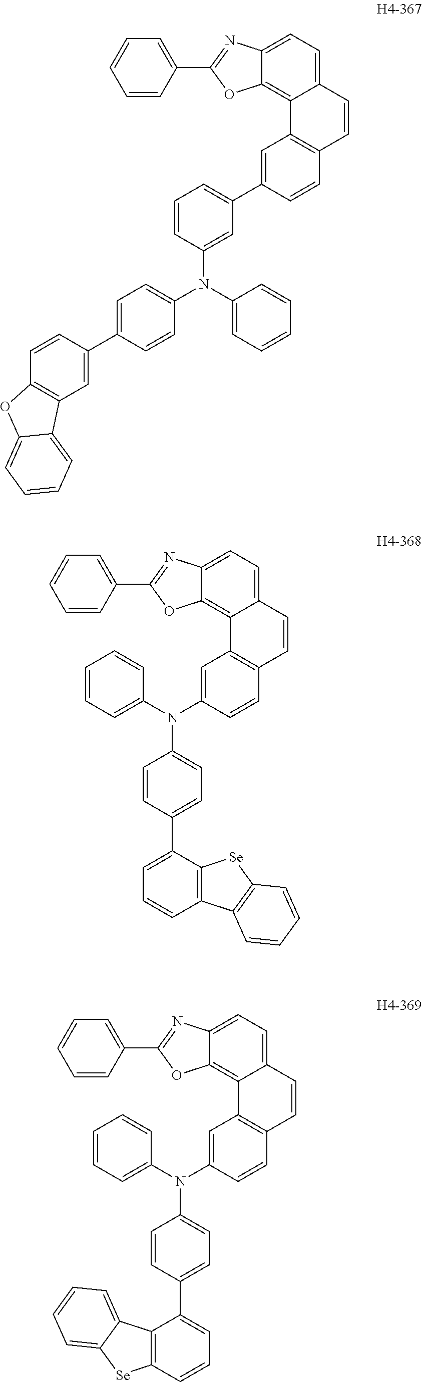 [SMILES: c1ccc(-c2nc3ccc4ccc5ccc(-c6cccc(N(c7ccccc7)c7ccc(-c8ccc9oc%10ccccc%10c9c8)cc7)c6)cc5c4c3o2)cc1 ; c1ccc(-c2nc3ccc4ccc5ccc(N(c6ccccc6)c6ccc(-c7cccc8c7[se]c7ccccc78)cc6)cc5c4c3o2)cc1 ; c1ccc(-c2nc3ccc4ccc5ccc(N(c6ccccc6)c6ccc(-c7cccc8[se]c9ccccc9c78)cc6)cc5c4c3o2)cc1]

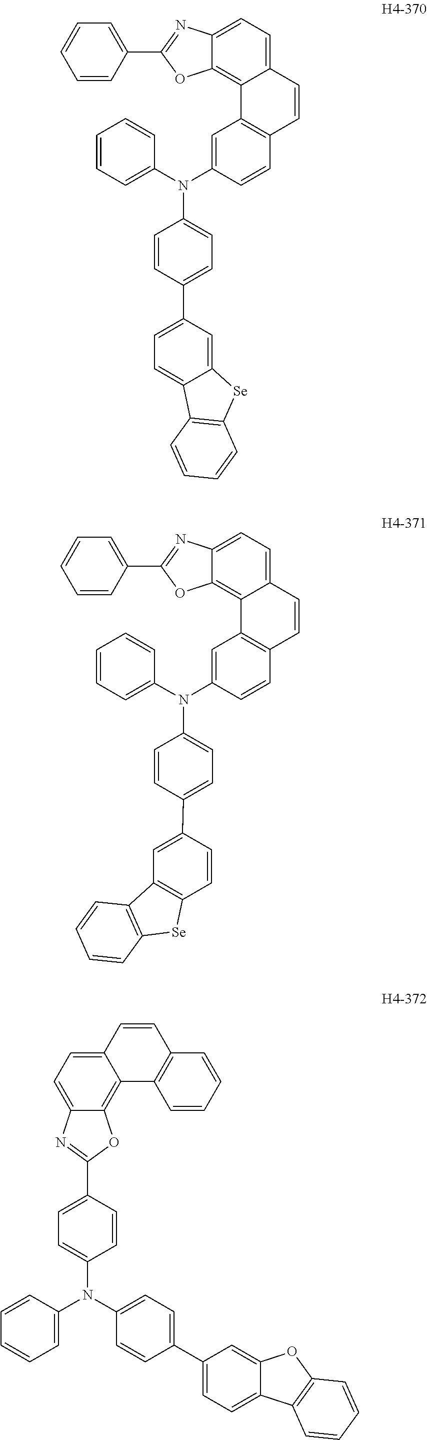 [SMILES: c1ccc(-c2nc3ccc4ccc5ccc(N(c6ccccc6)c6ccc(-c7ccc8c(c7)[se]c7ccccc78)cc6)cc5c4c3o2)cc1 ; c1ccc(-c2nc3ccc4ccc5ccc(N(c6ccccc6)c6ccc(-c7ccc8[se]c9ccccc9c8c7)cc6)cc5c4c3o2)cc1 ; c1ccc(N(c2ccc(-c3ccc4c(c3)oc3ccccc34)cc2)c2ccc(-c3nc4ccc5ccc6ccccc6c5c4o3)cc2)cc1]

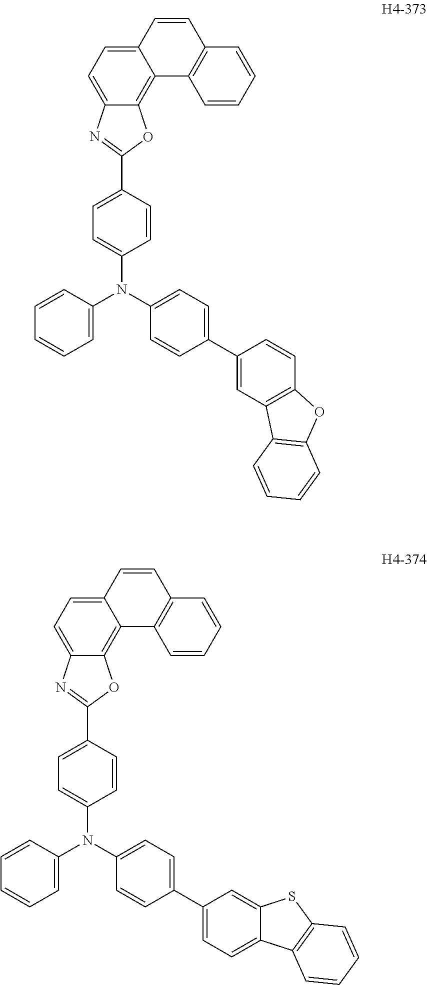 [SMILES: c1ccc(N(c2ccc(-c3ccc4oc5ccccc5c4c3)cc2)c2ccc(-c3nc4ccc5ccc6ccccc6c5c4o3)cc2)cc1 ; c1ccc(N(c2ccc(-c3ccc4c(c3)sc3ccccc34)cc2)c2ccc(-c3nc4ccc5ccc6ccccc6c5c4o3)cc2)cc1]

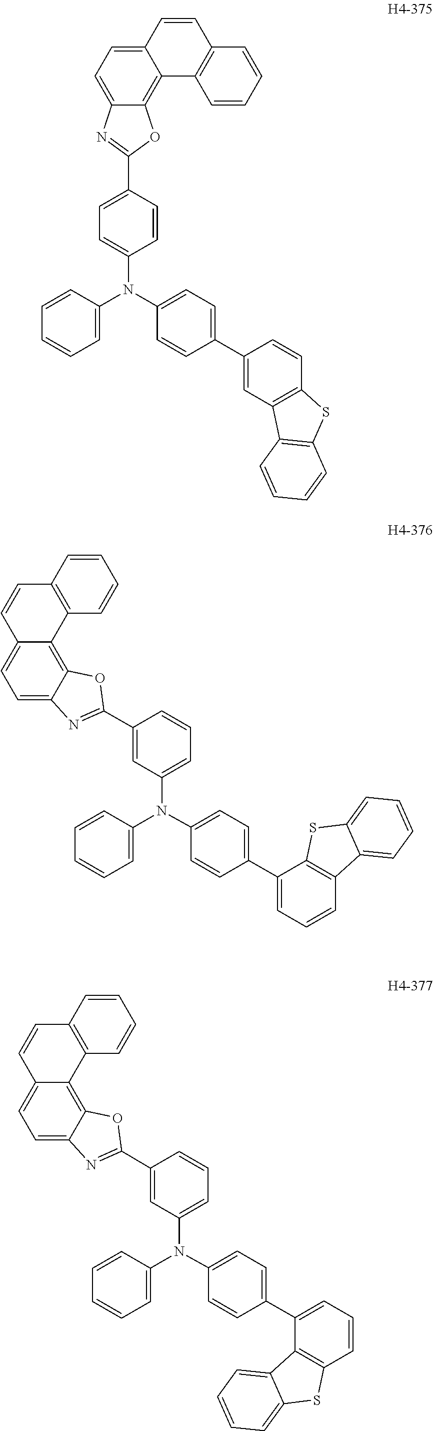 [SMILES: c1ccc(N(c2ccc(-c3ccc4sc5ccccc5c4c3)cc2)c2ccc(-c3nc4ccc5ccc6ccccc6c5c4o3)cc2)cc1 ; c1ccc(N(c2ccc(-c3cccc4c3sc3ccccc34)cc2)c2cccc(-c3nc4ccc5ccc6ccccc6c5c4o3)c2)cc1 ; c1ccc(N(c2ccc(-c3cccc4sc5ccccc5c34)cc2)c2cccc(-c3nc4ccc5ccc6ccccc6c5c4o3)c2)cc1]

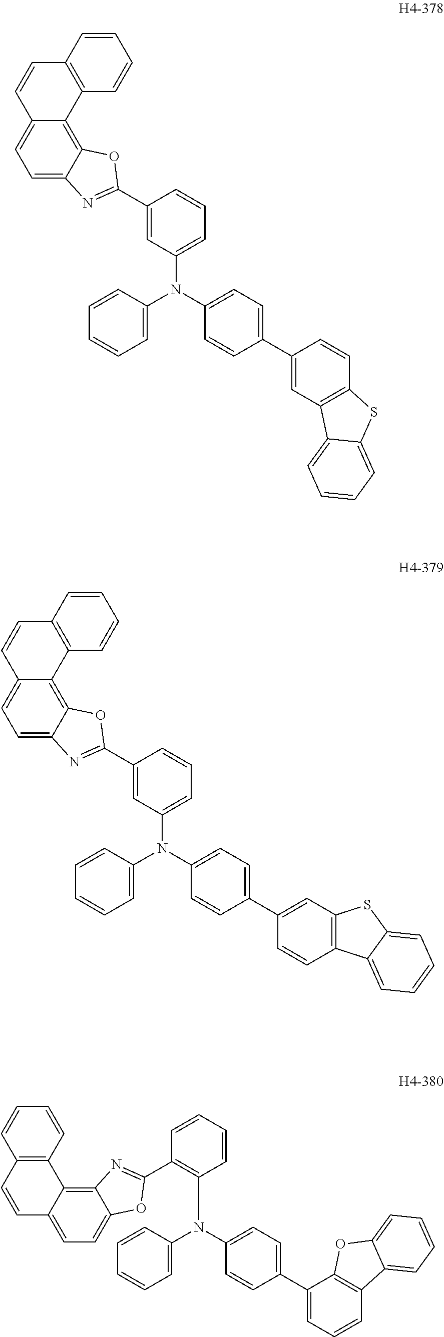 [SMILES: c1ccc(N(c2ccc(-c3ccc4sc5ccccc5c4c3)cc2)c2cccc(-c3nc4ccc5ccc6ccccc6c5c4o3)c2)cc1 ; c1ccc(N(c2ccc(-c3ccc4c(c3)sc3ccccc34)cc2)c2cccc(-c3nc4ccc5ccc6ccccc6c5c4o3)c2)cc1 ; c1ccc(N(c2ccc(-c3cccc4c3oc3ccccc34)cc2)c2ccccc2-c2nc3c(ccc4ccc5ccccc5c43)o2)cc1]

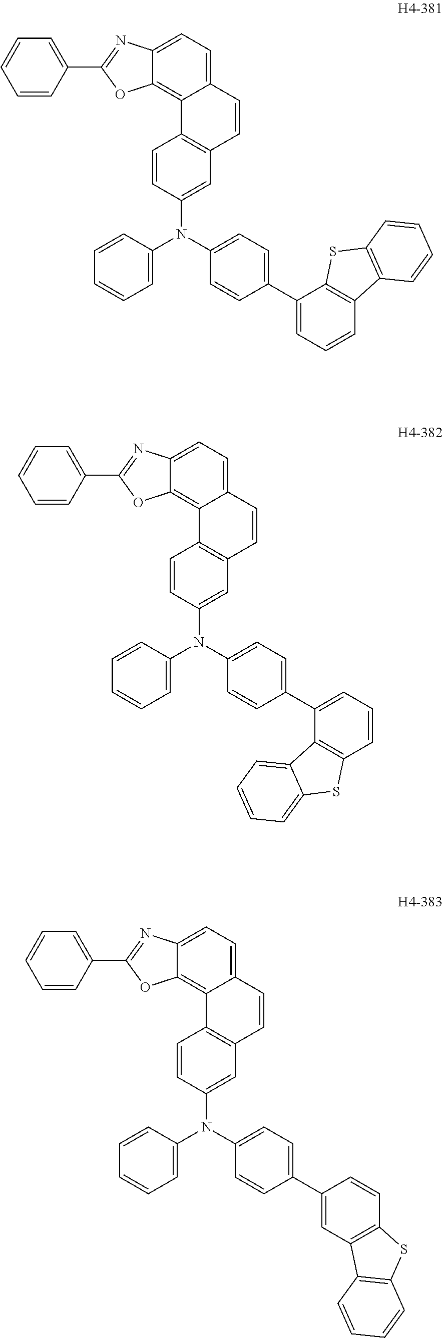 [SMILES: c1ccc(-c2nc3ccc4ccc5cc(N(c6ccccc6)c6ccc(-c7cccc8c7sc7ccccc78)cc6)ccc5c4c3o2)cc1 ; c1ccc(-c2nc3ccc4ccc5cc(N(c6ccccc6)c6ccc(-c7cccc8sc9ccccc9c78)cc6)ccc5c4c3o2)cc1 ; c1ccc(-c2nc3ccc4ccc5cc(N(c6ccccc6)c6ccc(-c7ccc8sc9ccccc9c8c7)cc6)ccc5c4c3o2)cc1]

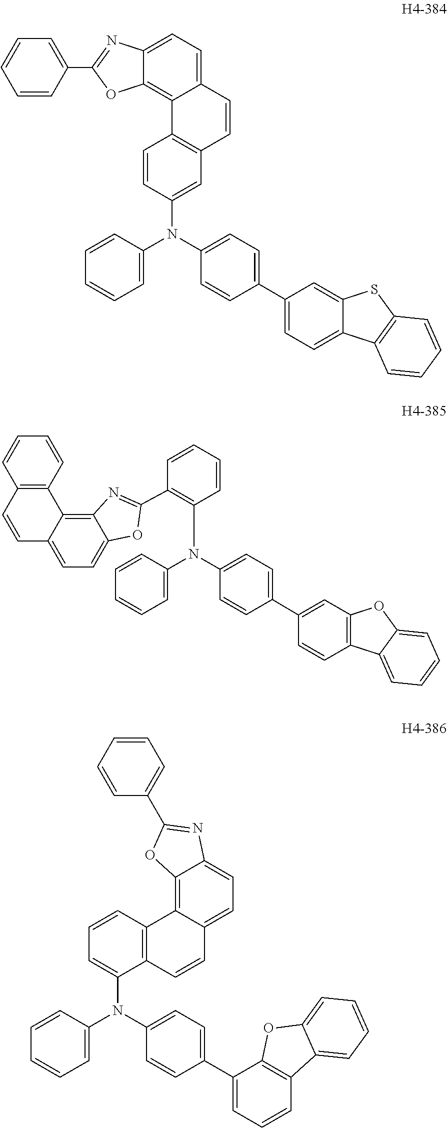 [SMILES: c1ccc(-c2nc3ccc4ccc5cc(N(c6ccccc6)c6ccc(-c7ccc8c(c7)sc7ccccc78)cc6)ccc5c4c3o2)cc1 ; c1ccc(N(c2ccc(-c3ccc4c(c3)oc3ccccc34)cc2)c2ccccc2-c2nc3c(ccc4ccc5ccccc5c43)o2)cc1 ; c1ccc(-c2nc3ccc4ccc5c(N(c6ccccc6)c6ccc(-c7cccc8c7oc7ccccc78)cc6)cccc5c4c3o2)cc1]

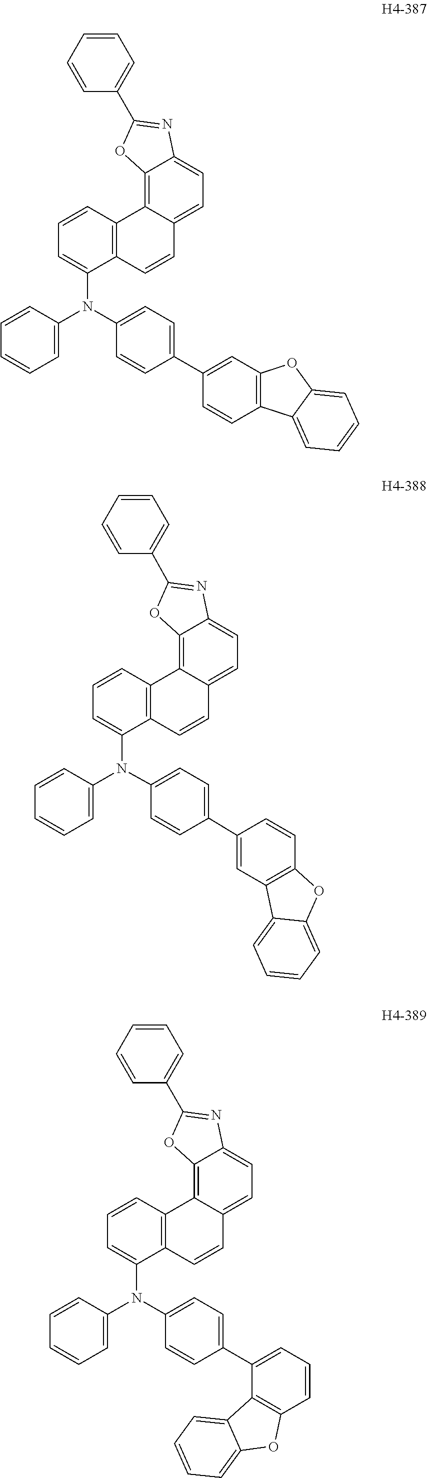 [SMILES: c1ccc(-c2nc3ccc4ccc5c(N(c6ccccc6)c6ccc(-c7ccc8c(c7)oc7ccccc78)cc6)cccc5c4c3o2)cc1 ; c1ccc(-c2nc3ccc4ccc5c(N(c6ccccc6)c6ccc(-c7ccc8oc9ccccc9c8c7)cc6)cccc5c4c3o2)cc1 ; c1ccc(-c2nc3ccc4ccc5c(N(c6ccccc6)c6ccc(-c7cccc8oc9ccccc9c78)cc6)cccc5c4c3o2)cc1]

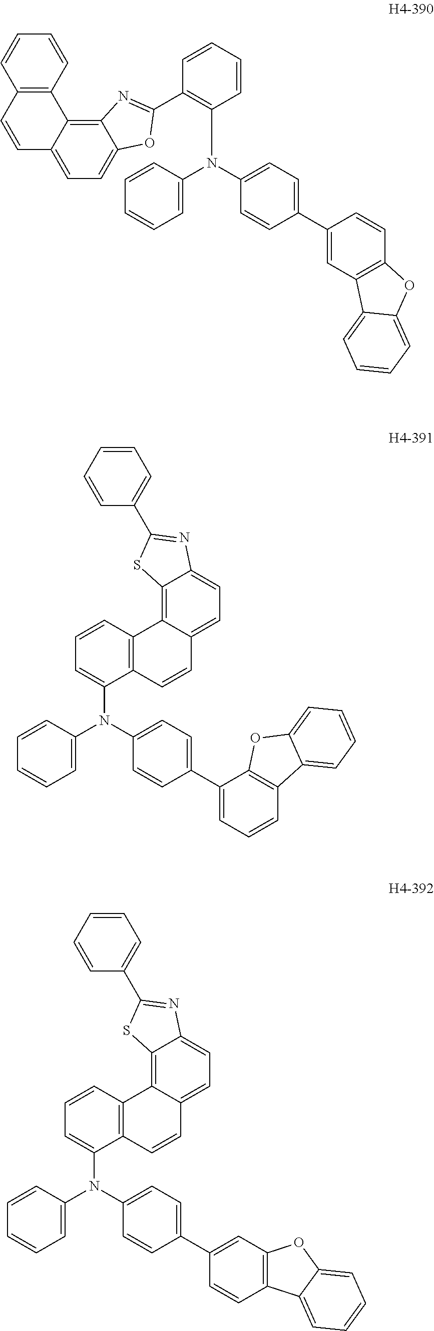 [SMILES: c1ccc(N(c2ccc(-c3ccc4oc5ccccc5c4c3)cc2)c2ccccc2-c2nc3c(ccc4ccc5ccccc5c43)o2)cc1 ; c1ccc(-c2nc3ccc4ccc5c(N(c6ccccc6)c6ccc(-c7cccc8c7oc7ccccc78)cc6)cccc5c4c3s2)cc1 ; c1ccc(-c2nc3ccc4ccc5c(N(c6ccccc6)c6ccc(-c7ccc8c(c7)oc7ccccc78)cc6)cccc5c4c3s2)cc1]

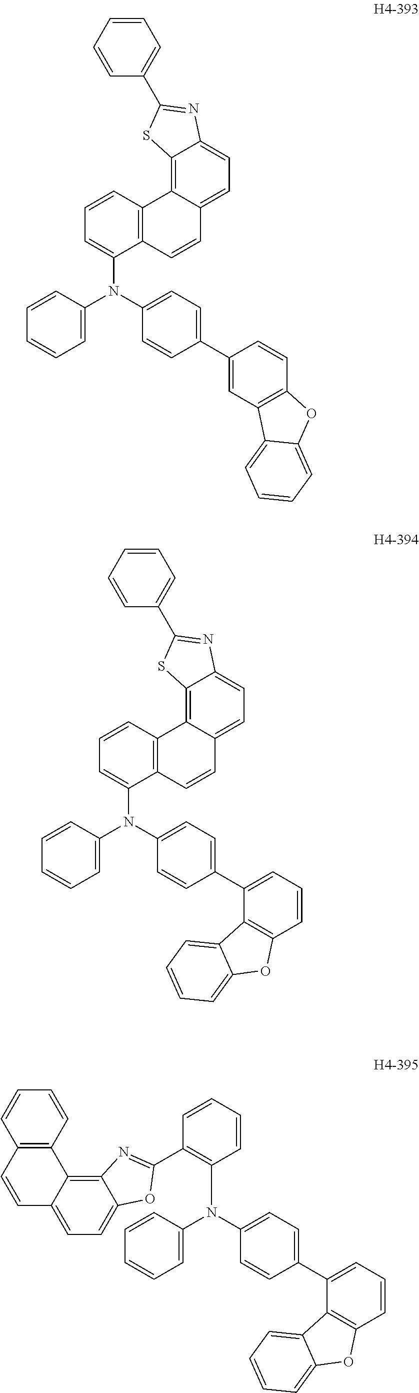 [SMILES: c1ccc(-c2nc3ccc4ccc5c(N(c6ccccc6)c6ccc(-c7ccc8oc9ccccc9c8c7)cc6)cccc5c4c3s2)cc1 ; c1ccc(-c2nc3ccc4ccc5c(N(c6ccccc6)c6ccc(-c7cccc8oc9ccccc9c78)cc6)cccc5c4c3s2)cc1 ; c1ccc(N(c2ccc(-c3cccc4oc5ccccc5c34)cc2)c2ccccc2-c2nc3c(ccc4ccc5ccccc5c43)o2)cc1]

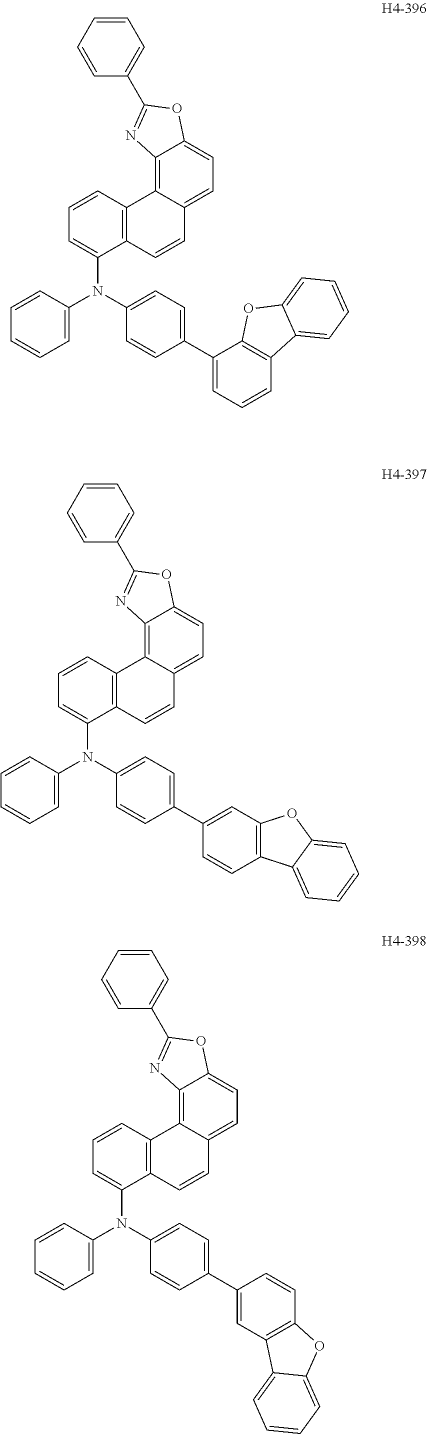 [SMILES: c1ccc(-c2nc3c(ccc4ccc5c(N(c6ccccc6)c6ccc(-c7cccc8c7oc7ccccc78)cc6)cccc5c43)o2)cc1 ; c1ccc(-c2nc3c(ccc4ccc5c(N(c6ccccc6)c6ccc(-c7ccc8c(c7)oc7ccccc78)cc6)cccc5c43)o2)cc1 ; c1ccc(-c2nc3c(ccc4ccc5c(N(c6ccccc6)c6ccc(-c7ccc8oc9ccccc9c8c7)cc6)cccc5c43)o2)cc1]

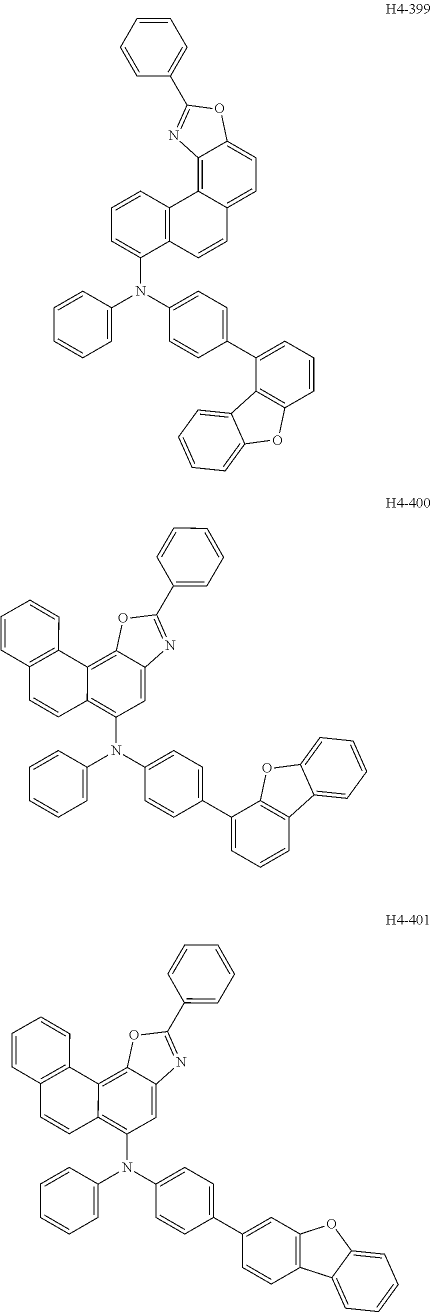 [SMILES: c1ccc(-c2nc3c(ccc4ccc5c(N(c6ccccc6)c6ccc(-c7cccc8oc9ccccc9c78)cc6)cccc5c43)o2)cc1 ; c1ccc(-c2nc3cc(N(c4ccccc4)c4ccc(-c5cccc6c5oc5ccccc56)cc4)c4ccc5ccccc5c4c3o2)cc1 ; c1ccc(-c2nc3cc(N(c4ccccc4)c4ccc(-c5ccc6c(c5)oc5ccccc56)cc4)c4ccc5ccccc5c4c3o2)cc1]

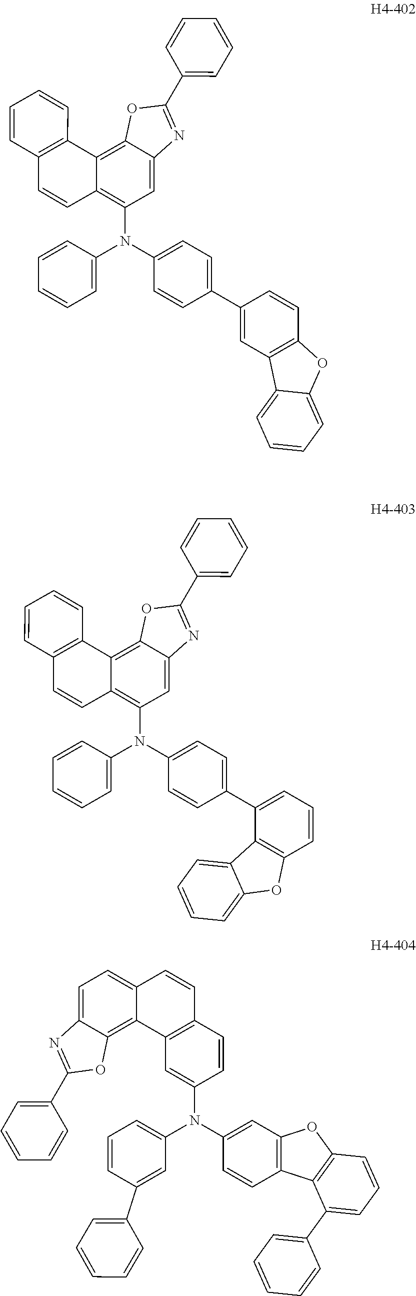 [SMILES: c1ccc(-c2nc3cc(N(c4ccccc4)c4ccc(-c5ccc6oc7ccccc7c6c5)cc4)c4ccc5ccccc5c4c3o2)cc1 ; c1ccc(-c2nc3cc(N(c4ccccc4)c4ccc(-c5cccc6oc7ccccc7c56)cc4)c4ccc5ccccc5c4c3o2)cc1 ; c1ccc(-c2cccc(N(c3ccc4c(c3)oc3cccc(-c5ccccc5)c34)c3ccc4ccc5ccc6nc(-c7ccccc7)oc6c5c4c3)c2)cc1]

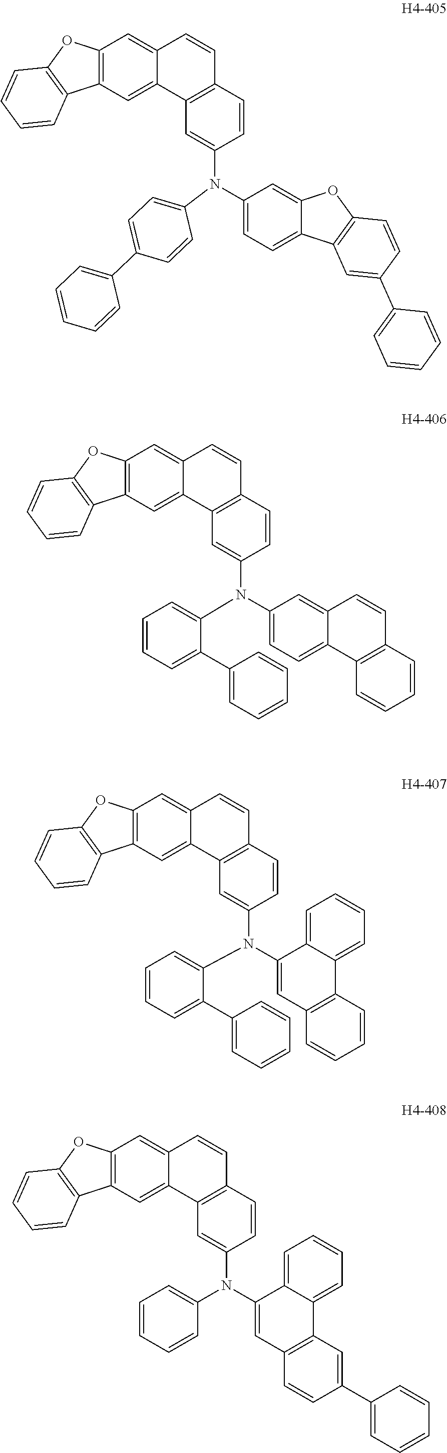 [SMILES: c1ccc(-c2ccc(N(c3ccc4c(c3)oc3ccc(-c5ccccc5)cc34)c3ccc4ccc5cc6oc7ccccc7c6cc5c4c3)cc2)cc1 ; c1ccc(-c2ccccc2N(c2ccc3c(ccc4ccccc43)c2)c2ccc3ccc4cc5oc6ccccc6c5cc4c3c2)cc1 ; c1ccc(-c2ccccc2N(c2ccc3ccc4cc5oc6ccccc6c5cc4c3c2)c2cc3ccccc3c3ccccc23)cc1 ; c1ccc(-c2ccc3cc(N(c4ccccc4)c4ccc5ccc6cc7oc8ccccc8c7cc6c5c4)c4ccccc4c3c2)cc1]

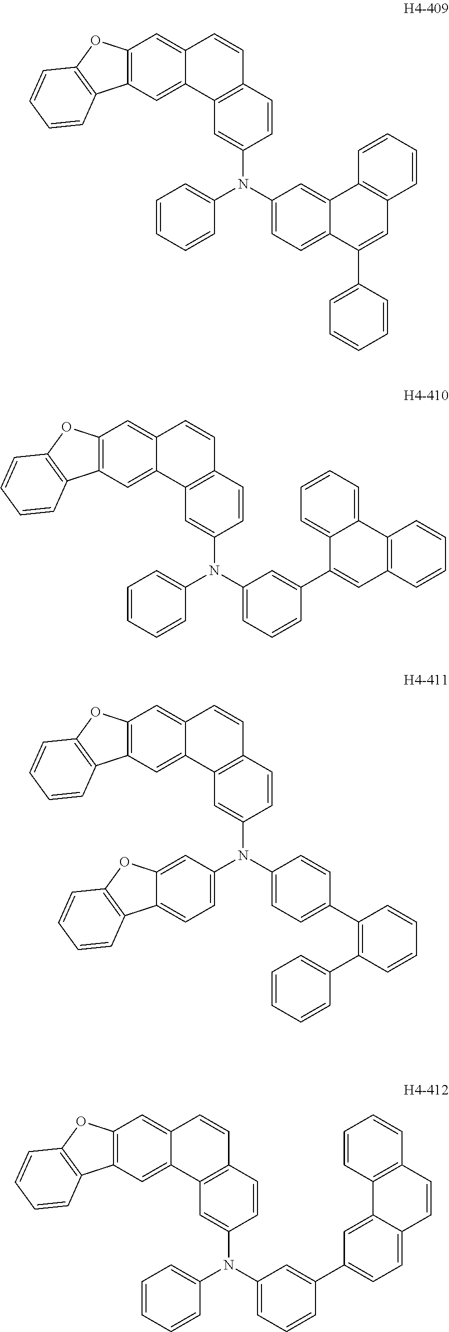 [SMILES: c1ccc(-c2cc3ccccc3c3cc(N(c4ccccc4)c4ccc5ccc6cc7oc8ccccc8c7cc6c5c4)ccc23)cc1 ; c1ccc(N(c2cccc(-c3cc4ccccc4c4ccccc34)c2)c2ccc3ccc4cc5oc6ccccc6c5cc4c3c2)cc1 ; c1ccc(-c2ccccc2-c2ccc(N(c3ccc4c(c3)oc3ccccc34)c3ccc4ccc5cc6oc7ccccc7c6cc5c4c3)cc2)cc1 ; c1ccc(N(c2cccc(-c3ccc4ccc5ccccc5c4c3)c2)c2ccc3ccc4cc5oc6ccccc6c5cc4c3c2)cc1]

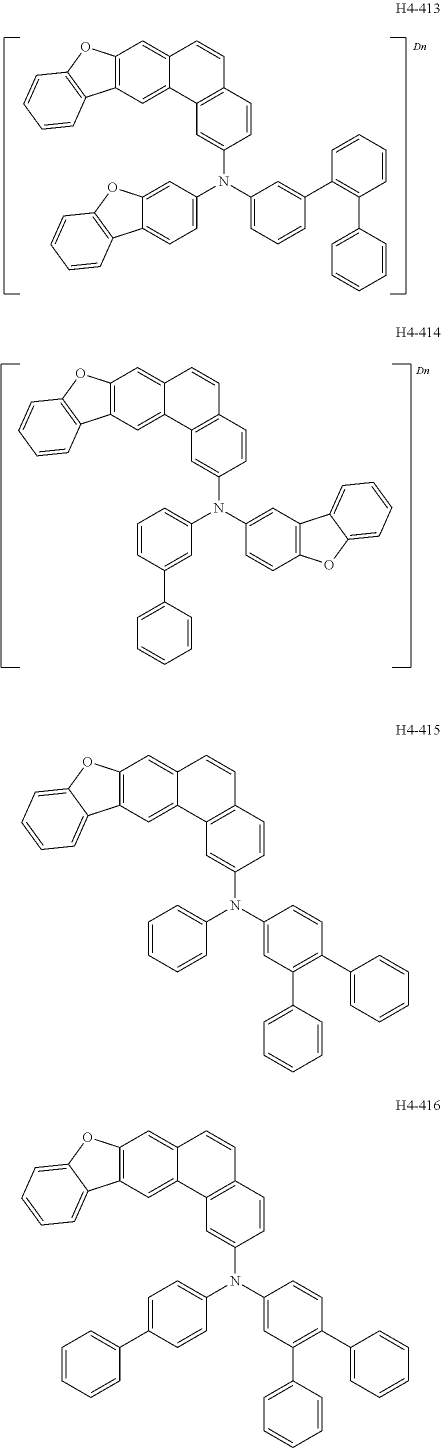

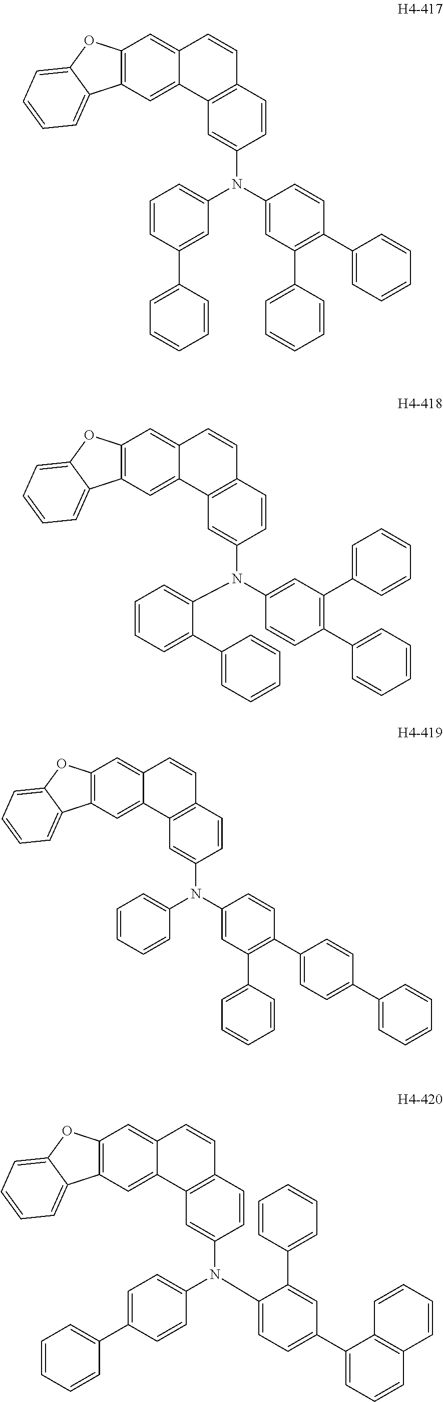 [SMILES: c1ccc(-c2cccc(N(c3ccc(-c4ccccc4)c(-c4ccccc4)c3)c3ccc4ccc5cc6oc7ccccc7c6cc5c4c3)c2)cc1 ; c1ccc(-c2ccc(N(c3ccc4ccc5cc6oc7ccccc7c6cc5c4c3)c3ccccc3-c3ccccc3)cc2-c2ccccc2)cc1 ; c1ccc(-c2ccc(-c3ccc(N(c4ccccc4)c4ccc5ccc6cc7oc8ccccc8c7cc6c5c4)cc3-c3ccccc3)cc2)cc1 ; c1ccc(-c2ccc(N(c3ccc4ccc5cc6oc7ccccc7c6cc5c4c3)c3ccc(-c4cccc5ccccc45)cc3-c3ccccc3)cc2)cc1]

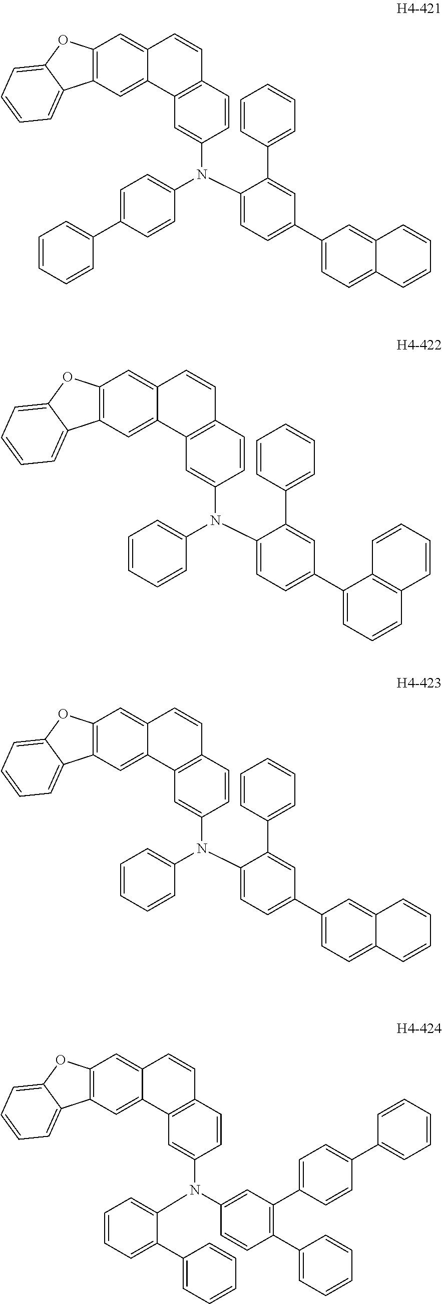 [SMILES: c1ccc(-c2ccc(N(c3ccc4ccc5cc6oc7ccccc7c6cc5c4c3)c3ccc(-c4ccc5ccccc5c4)cc3-c3ccccc3)cc2)cc1 ; c1ccc(-c2cc(-c3cccc4ccccc34)ccc2N(c2ccccc2)c2ccc3ccc4cc5oc6ccccc6c5cc4c3c2)cc1 ; c1ccc(-c2cc(-c3ccc4ccccc4c3)ccc2N(c2ccccc2)c2ccc3ccc4cc5oc6ccccc6c5cc4c3c2)cc1 ; c1ccc(-c2ccc(-c3cc(N(c4ccc5ccc6cc7oc8ccccc8c7cc6c5c4)c4ccccc4-c4ccccc4)ccc3-c3ccccc3)cc2)cc1]

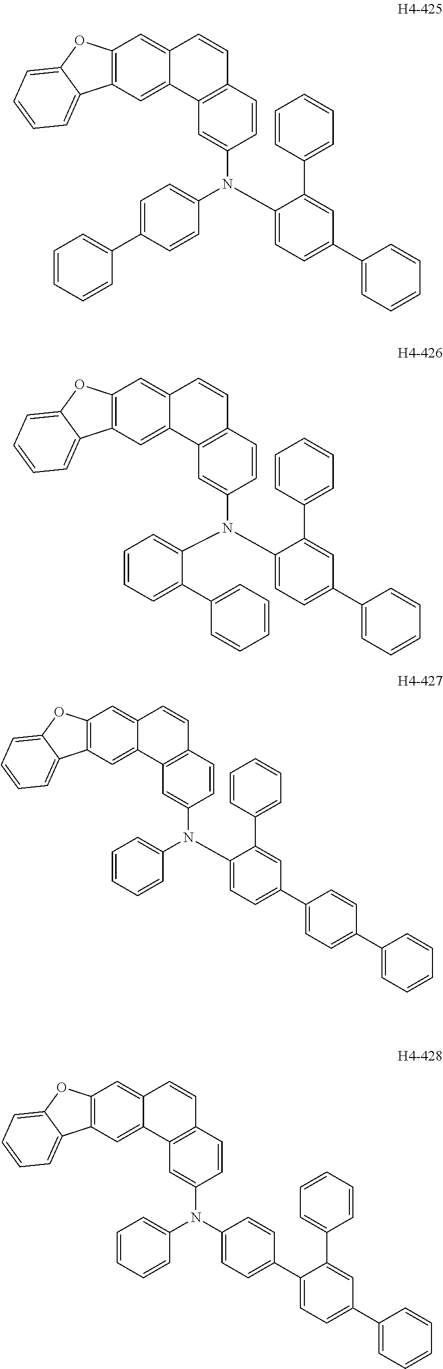 [SMILES: c1ccc(-c2ccc(N(c3ccc4ccc5cc6oc7ccccc7c6cc5c4c3)c3ccc(-c4ccccc4)cc3-c3ccccc3)cc2)cc1 ; c1ccc(-c2ccc(N(c3ccc4ccc5cc6oc7ccccc7c6cc5c4c3)c3ccccc3-c3ccccc3)c(-c3ccccc3)c2)cc1 ; c1ccc(-c2ccc(-c3ccc(N(c4ccccc4)c4ccc5ccc6cc7oc8ccccc8c7cc6c5c4)c(-c4ccccc4)c3)cc2)cc1 ; c1ccc(-c2ccc(-c3ccc(N(c4ccccc4)c4ccc5ccc6cc7oc8ccccc8c7cc6c5c4)cc3)c(-c3ccccc3)c2)cc1]

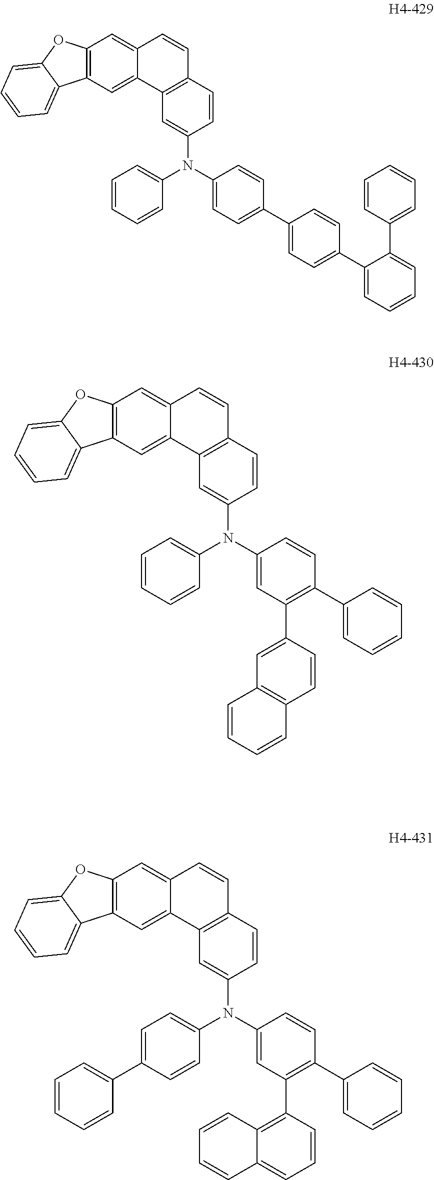 [SMILES: c1ccc(-c2ccccc2-c2ccc(-c3ccc(N(c4ccccc4)c4ccc5ccc6cc7oc8ccccc8c7cc6c5c4)cc3)cc2)cc1 ; c1ccc(-c2ccc(N(c3ccccc3)c3ccc4ccc5cc6oc7ccccc7c6cc5c4c3)cc2-c2ccc3ccccc3c2)cc1 ; c1ccc(-c2ccc(N(c3ccc(-c4ccccc4)c(-c4cccc5ccccc45)c3)c3ccc4ccc5cc6oc7ccccc7c6cc5c4c3)cc2)cc1]

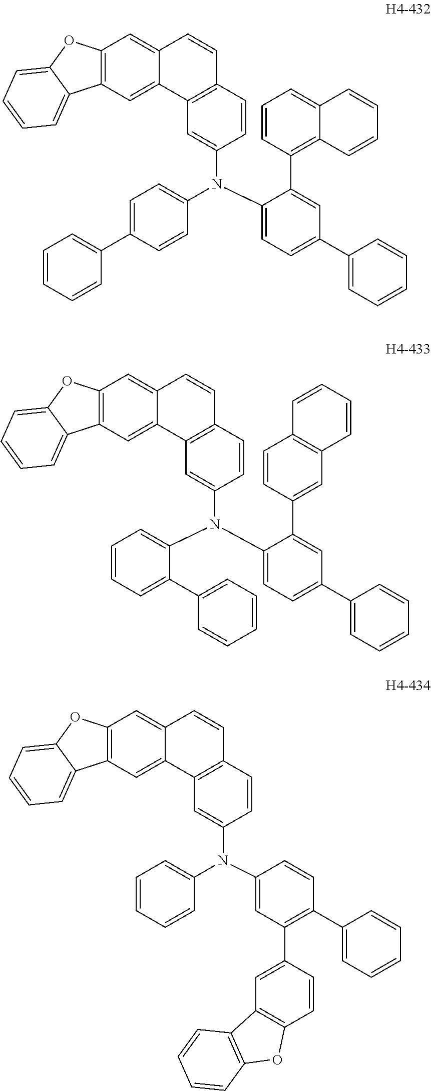 [SMILES: c1ccc(-c2ccc(N(c3ccc4ccc5cc6oc7ccccc7c6cc5c4c3)c3ccc(-c4ccccc4)cc3-c3cccc4ccccc34)cc2)cc1 ; c1ccc(-c2ccc(N(c3ccc4ccc5cc6oc7ccccc7c6cc5c4c3)c3ccccc3-c3ccccc3)c(-c3ccc4ccccc4c3)c2)cc1 ; c1ccc(-c2ccc(N(c3ccccc3)c3ccc4ccc5cc6oc7ccccc7c6cc5c4c3)cc2-c2ccc3oc4ccccc4c3c2)cc1]

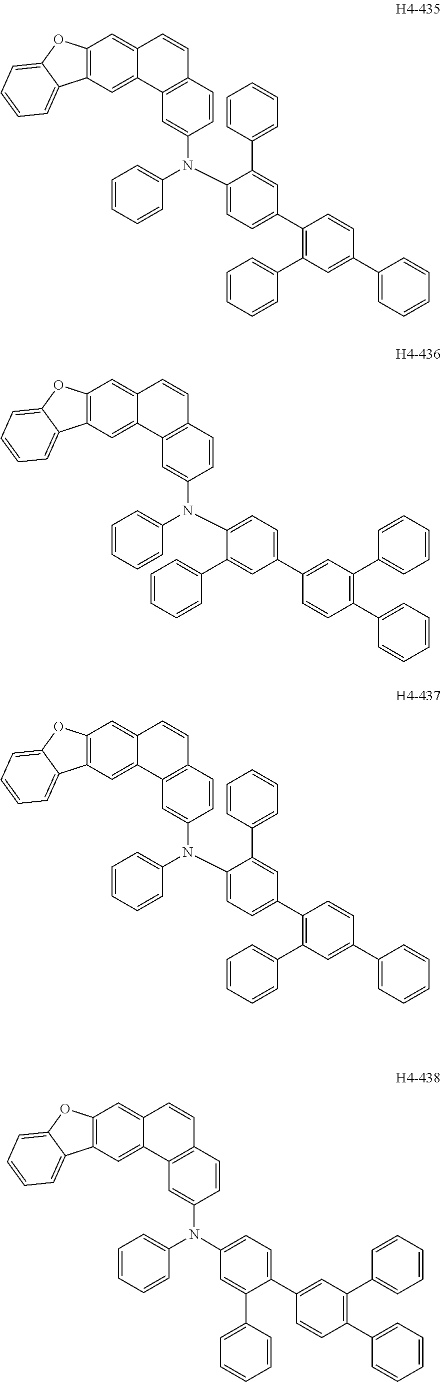 [SMILES: c1ccc(-c2ccc(-c3ccc(N(c4ccccc4)c4ccc5ccc6cc7oc8ccccc8c7cc6c5c4)c(-c4ccccc4)c3)c(-c3ccccc3)c2)cc1 ; c1ccc(-c2ccc(-c3ccc(N(c4ccccc4)c4ccc5ccc6cc7oc8ccccc8c7cc6c5c4)c(-c4ccccc4)c3)cc2-c2ccccc2)cc1 ; c1ccc(-c2ccc(-c3ccc(N(c4ccccc4)c4ccc5ccc6cc7oc8ccccc8c7cc6c5c4)c(-c4ccccc4)c3)c(-c3ccccc3)c2)cc1 ; c1ccc(-c2ccc(-c3ccc(N(c4ccccc4)c4ccc5ccc6cc7oc8ccccc8c7cc6c5c4)cc3-c3ccccc3)cc2-c2ccccc2)cc1]

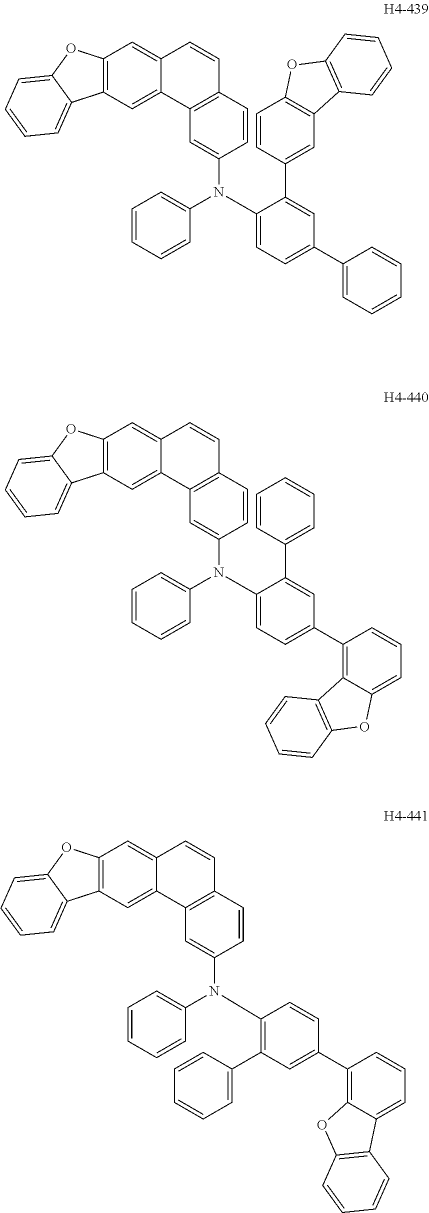 [SMILES: c1ccc(-c2ccc(N(c3ccccc3)c3ccc4ccc5cc6oc7ccccc7c6cc5c4c3)c(-c3ccc4oc5ccccc5c4c3)c2)cc1 ; c1ccc(-c2cc(-c3cccc4oc5ccccc5c34)ccc2N(c2ccccc2)c2ccc3ccc4cc5oc6ccccc6c5cc4c3c2)cc1 ; c1ccc(-c2cc(-c3cccc4c3oc3ccccc34)ccc2N(c2ccccc2)c2ccc3ccc4cc5oc6ccccc6c5cc4c3c2)cc1]

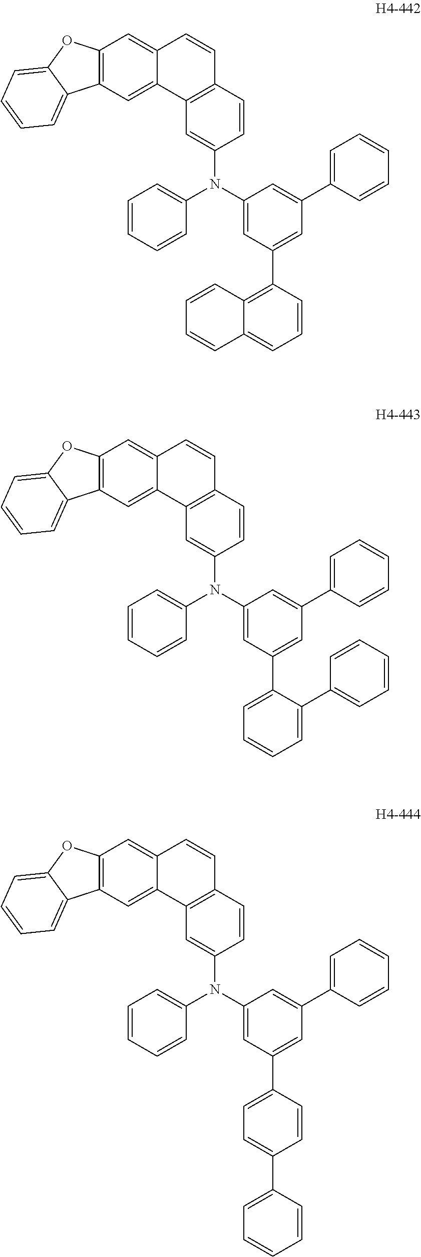 [SMILES: c1ccc(-c2cc(-c3cccc4ccccc34)cc(N(c3ccccc3)c3ccc4ccc5cc6oc7ccccc7c6cc5c4c3)c2)cc1 ; c1ccc(-c2cc(-c3ccccc3-c3ccccc3)cc(N(c3ccccc3)c3ccc4ccc5cc6oc7ccccc7c6cc5c4c3)c2)cc1 ; c1ccc(-c2ccc(-c3cc(-c4ccccc4)cc(N(c4ccccc4)c4ccc5ccc6cc7oc8ccccc8c7cc6c5c4)c3)cc2)cc1]

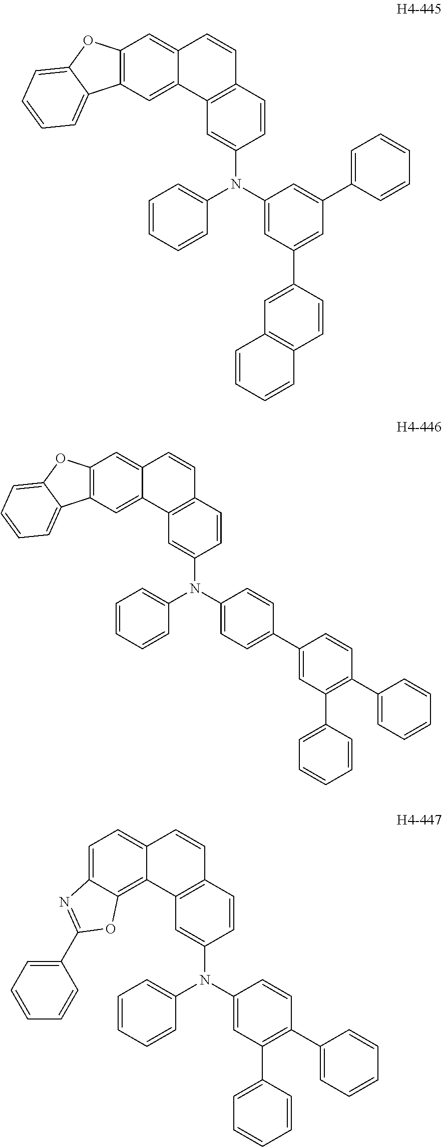 [SMILES: c1ccc(-c2cc(-c3ccc4ccccc4c3)cc(N(c3ccccc3)c3ccc4ccc5cc6oc7ccccc7c6cc5c4c3)c2)cc1 ; c1ccc(-c2ccc(-c3ccc(N(c4ccccc4)c4ccc5ccc6cc7oc8ccccc8c7cc6c5c4)cc3)cc2-c2ccccc2)cc1 ; c1ccc(-c2nc3ccc4ccc5ccc(N(c6ccccc6)c6ccc(-c7ccccc7)c(-c7ccccc7)c6)cc5c4c3o2)cc1]

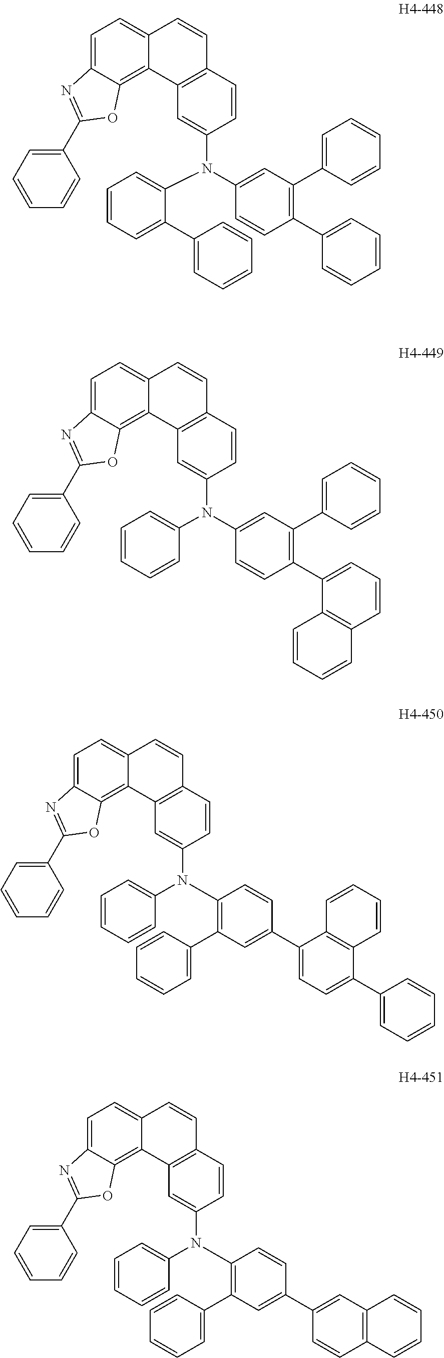 [SMILES: c1ccc(-c2nc3ccc4ccc5ccc(N(c6ccc(-c7ccccc7)c(-c7ccccc7)c6)c6ccccc6-c6ccccc6)cc5c4c3o2)cc1 ; c1ccc(-c2nc3ccc4ccc5ccc(N(c6ccccc6)c6ccc(-c7cccc8ccccc78)c(-c7ccccc7)c6)cc5c4c3o2)cc1 ; c1ccc(-c2nc3ccc4ccc5ccc(N(c6ccccc6)c6ccc(-c7ccc(-c8ccccc8)c8ccccc78)cc6-c6ccccc6)cc5c4c3o2)cc1 ; c1ccc(-c2nc3ccc4ccc5ccc(N(c6ccccc6)c6ccc(-c7ccc8ccccc8c7)cc6-c6ccccc6)cc5c4c3o2)cc1]

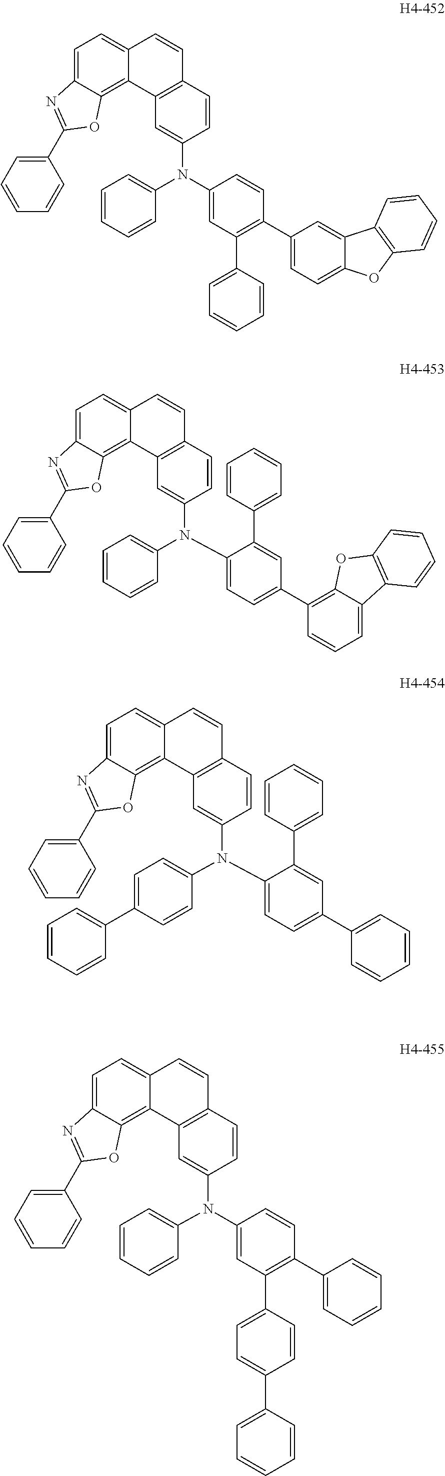 [SMILES: c1ccc(-c2nc3ccc4ccc5ccc(N(c6ccccc6)c6ccc(-c7ccc8oc9ccccc9c8c7)c(-c7ccccc7)c6)cc5c4c3o2)cc1 ; c1ccc(-c2nc3ccc4ccc5ccc(N(c6ccccc6)c6ccc(-c7cccc8c7oc7ccccc78)cc6-c6ccccc6)cc5c4c3o2)cc1 ; c1ccc(-c2ccc(N(c3ccc4ccc5ccc6nc(-c7ccccc7)oc6c5c4c3)c3ccc(-c4ccccc4)cc3-c3ccccc3)cc2)cc1 ; c1ccc(-c2ccc(-c3cc(N(c4ccccc4)c4ccc5ccc6ccc7nc(-c8ccccc8)oc7c6c5c4)ccc3-c3ccccc3)cc2)cc1]

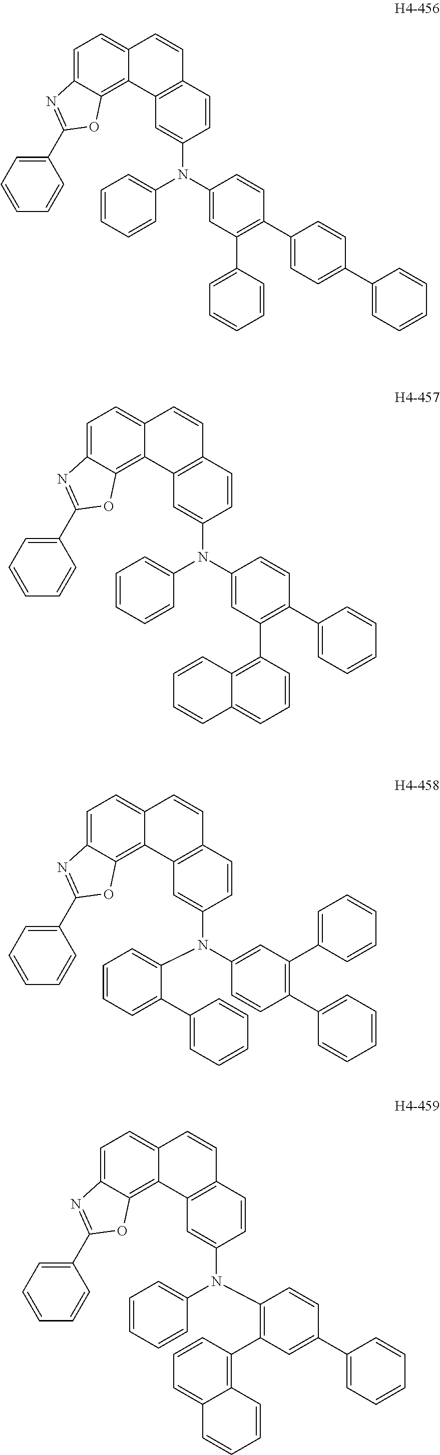 [SMILES: c1ccc(-c2ccc(-c3ccc(N(c4ccccc4)c4ccc5ccc6ccc7nc(-c8ccccc8)oc7c6c5c4)cc3-c3ccccc3)cc2)cc1 ; c1ccc(-c2nc3ccc4ccc5ccc(N(c6ccccc6)c6ccc(-c7ccccc7)c(-c7cccc8ccccc78)c6)cc5c4c3o2)cc1 ; c1ccc(-c2nc3ccc4ccc5ccc(N(c6ccc(-c7ccccc7)c(-c7ccccc7)c6)c6ccccc6-c6ccccc6)cc5c4c3o2)cc1 ; c1ccc(-c2ccc(N(c3ccccc3)c3ccc4ccc5ccc6nc(-c7ccccc7)oc6c5c4c3)c(-c3cccc4ccccc34)c2)cc1]

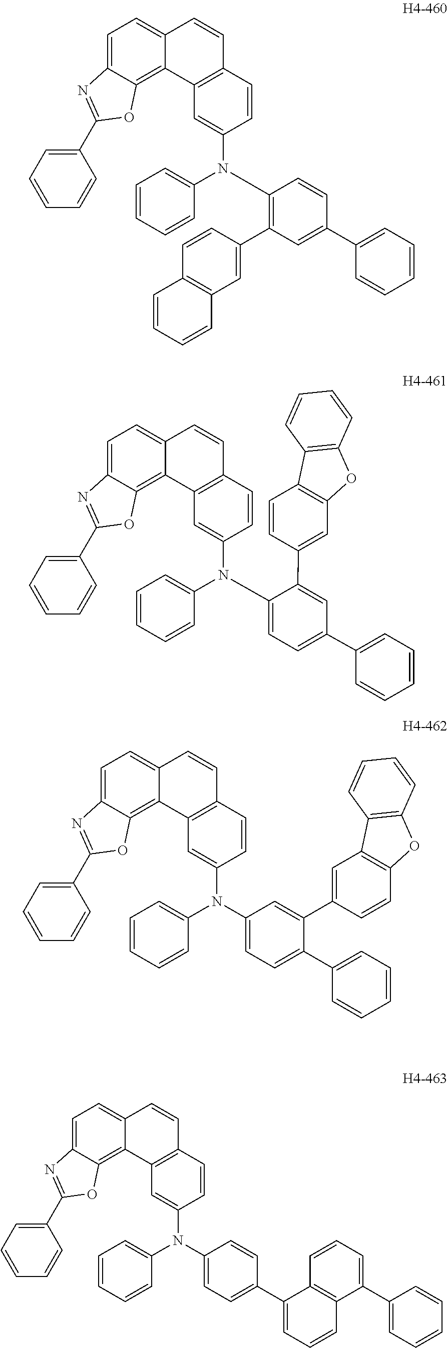 [SMILES: c1ccc(-c2ccc(N(c3ccccc3)c3ccc4ccc5ccc6nc(-c7ccccc7)oc6c5c4c3)c(-c3ccc4ccccc4c3)c2)cc1 ; c1ccc(-c2ccc(N(c3ccccc3)c3ccc4ccc5ccc6nc(-c7ccccc7)oc6c5c4c3)c(-c3ccc4c(c3)oc3ccccc34)c2)cc1 ; c1ccc(-c2nc3ccc4ccc5ccc(N(c6ccccc6)c6ccc(-c7ccccc7)c(-c7ccc8oc9ccccc9c8c7)c6)cc5c4c3o2)cc1 ; c1ccc(-c2nc3ccc4ccc5ccc(N(c6ccccc6)c6ccc(-c7cccc8c(-c9ccccc9)cccc78)cc6)cc5c4c3o2)cc1]

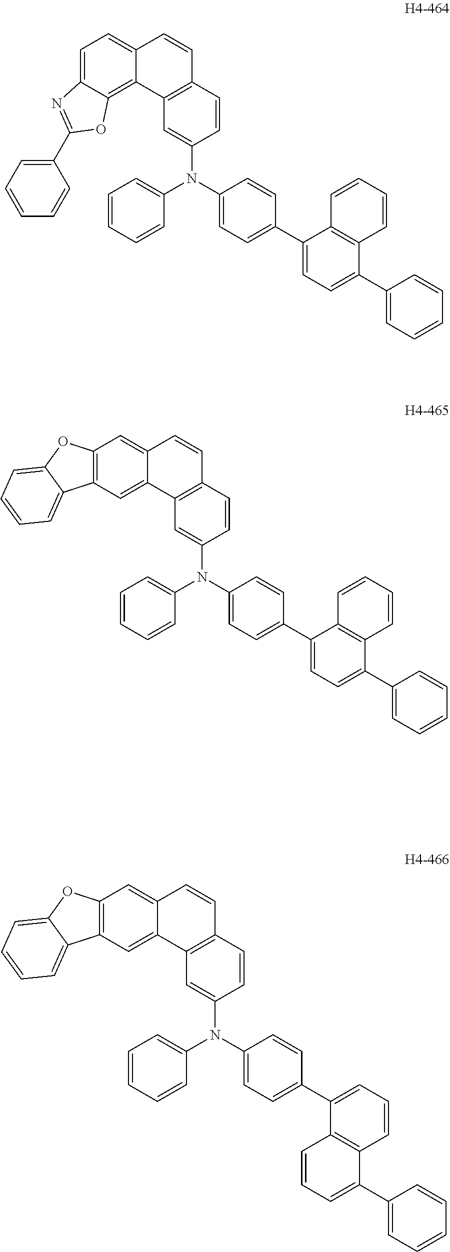 [SMILES: c1ccc(-c2nc3ccc4ccc5ccc(N(c6ccccc6)c6ccc(-c7ccc(-c8ccccc8)c8ccccc78)cc6)cc5c4c3o2)cc1 ; c1ccc(-c2ccc(-c3ccc(N(c4ccccc4)c4ccc5ccc6cc7oc8ccccc8c7cc6c5c4)cc3)c3ccccc23)cc1 ; c1ccc(-c2cccc3c(-c4ccc(N(c5ccccc5)c5ccc6ccc7cc8oc9ccccc9c8cc7c6c5)cc4)cccc23)cc1]

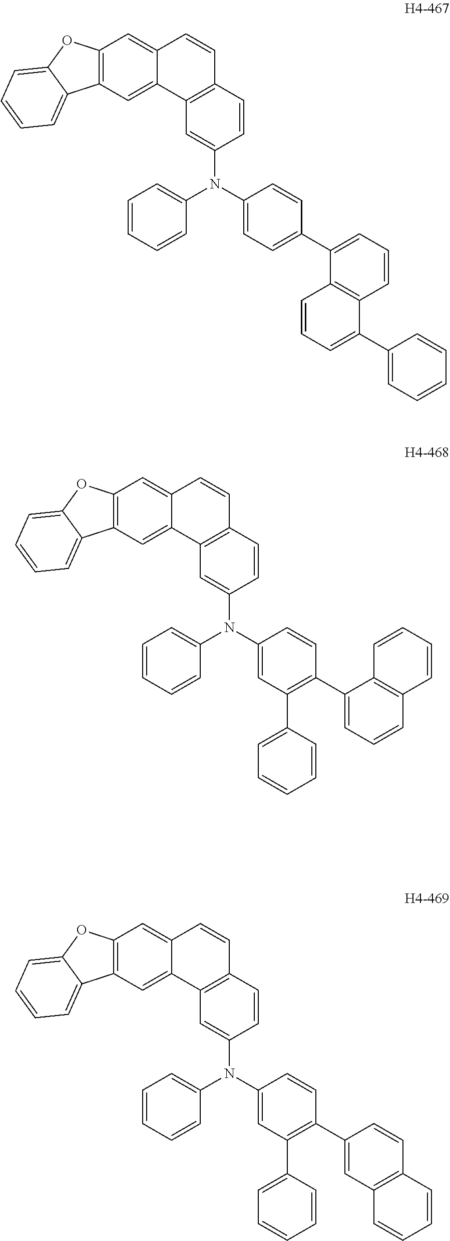 [SMILES: c1ccc(-c2cccc3c(-c4ccc(N(c5ccccc5)c5ccc6ccc7cc8oc9ccccc9c8cc7c6c5)cc4)cccc23)cc1 ; c1ccc(-c2cc(N(c3ccccc3)c3ccc4ccc5cc6oc7ccccc7c6cc5c4c3)ccc2-c2cccc3ccccc23)cc1 ; c1ccc(-c2cc(N(c3ccccc3)c3ccc4ccc5cc6oc7ccccc7c6cc5c4c3)ccc2-c2ccc3ccccc3c2)cc1]

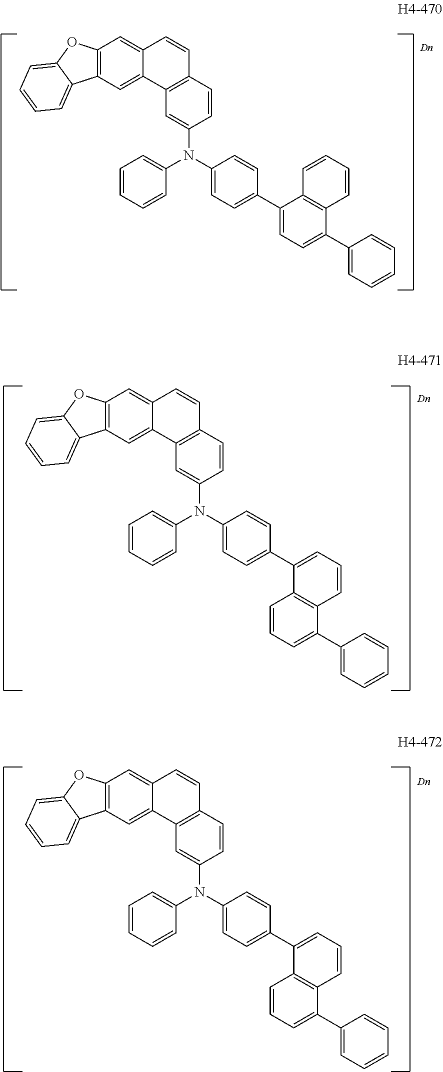

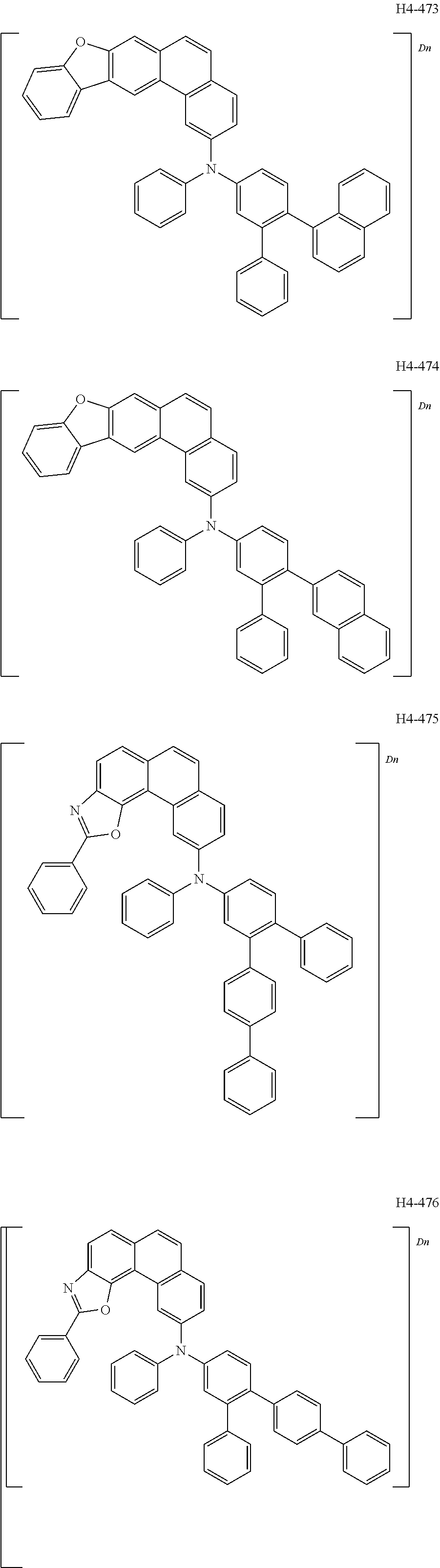

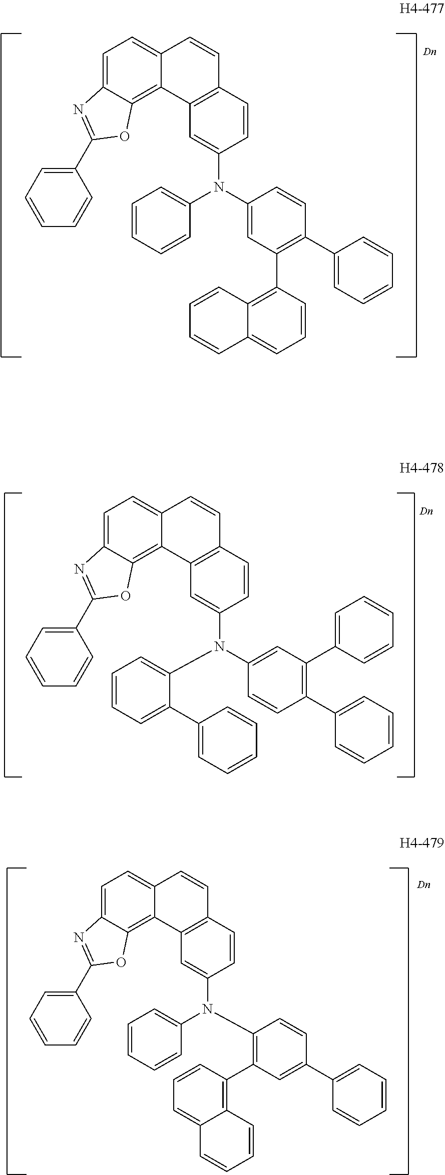

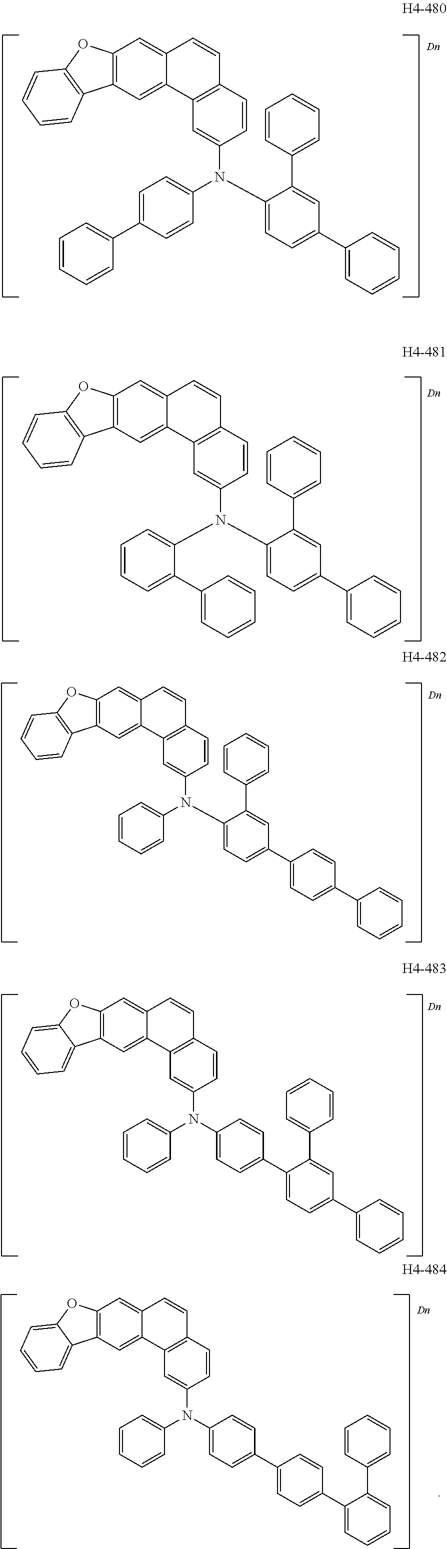

In the compounds above, Dₙ means that n number of hydrogens is replaced with deuterium, wherein n is an integer of 1 or more, and the upper limit of n is determined according to the number of hydrogens that may be substituted for each compound.

The compound represented by Formula 2 according to the present disclosure can be prepared by referring to synthetic methods known to those skilled in the art, for example, synthetic methods disclosed in Korean Patent Application Laid-Open Nos. 2018-0099487, 2021-0098316, and 2022-0051794, etc., but is not limited thereto.

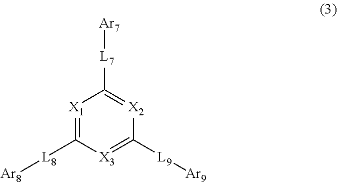

In Formula 3,
X₁ to X₃ each independently represent N or CR₁₁; provided that at least one of X₁ to X₃ is N;
L₇ to L₉ each independently represent a single bond, a substituted or unsubstituted (C6-C30)arylene, or a substituted or unsubstituted (3- to 30-membered)heteroarylene;
R₁₁ represents hydrogen, deuterium, a halogen, a cyano, a substituted or unsubstituted (C1-C30)alkyl, a substituted or unsubstituted (C6-C30)aryl, a substituted or unsubstituted (3-to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring of (C3-C30) aliphatic ring and (C6-C30)aromatic ring;
Ar₇ to Ar₉ each independently represent a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring of (C3-C30) aliphatic ring and (C6-C30)aromatic ring, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₂₁-N(Ar₂₁)(Ar₂₂);
L₂₁ represents a single bond, a substituted or unsubstituted (C6-C30)arylene, or a substituted or unsubstituted (3- to 30-membered)heteroarylene; and
Ar₂₁ and Ar₂₂ each independently represent a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl.

In one embodiment, at least two of X₁ to X₃ may be N.

In one embodiment, all of X₁ to X₃ may be N.

In one embodiment, L₇ to L₉ each independently may be a single bond, a substituted or unsubstituted (C6-C30)arylene, or a substituted or unsubstituted (5- to 30-membered)heteroarylene, preferably a single bond, a substituted or unsubstituted (C6-C25)arylene, or a substituted or unsubstituted (5- to 25-membered)heteroarylene, more preferably a single bond, a substituted or unsubstituted (C6-C18)arylene or a substituted or unsubstituted (5- to 18-membered)heteroarylene. For example, L₇ to L₉ each independently represent a single bond, a substituted or unsubstituted phenylene, a substituted or unsubstituted naphthylene, a substituted or unsubstituted biphenylene, a substituted or unsubstituted naphthylphenylene, a substituted or unsubstituted phenylnaphthylene, a substituted or unsubstituted phenanthrenylene, a substituted or unsubstituted dibenzofuranylene, or a substituted or unsubstituted dibenzothiophenylene, wherein the substituents, for example, may be substituted with at least one deuterium.

In one embodiment, Ar₇ to Ar₉ each independently may be a substituted or unsubstituted (C6-C30)aryl, or a substituted or unsubstituted (5- to 30-membered)heteroaryl, preferably a substituted or unsubstituted (C6-C25)aryl, or a substituted or unsubstituted (5- to 25-membered)heteroaryl, more preferably a substituted or unsubstituted (C6-C25)aryl, or a substituted or unsubstituted (5- to 18-membered)heteroaryl.

In one embodiment, at least one of Ar₇ to Ar₉ may be a substituted or unsubstituted (5-to 30-membered)heteroaryl, preferably at least two of Ar₇ to Ar₉ may be a substituted or unsubstituted (5- to 30-membered)heteroaryl. For example, Ar₇ to Ar₉ each independently may be a substituted or unsubstituted phenyl, a substituted or unsubstituted naphthyl, a substituted or unsubstituted p-biphenyl, a substituted or unsubstituted m-biphenyl, a substituted or unsubstituted o-terphenyl, a substituted or unsubstituted m-terphenyl, a substituted or unsubstituted p-terphenyl, a substituted or unsubstituted triphenylsilyl, a substituted or unsubstituted dibenzofuranyl, a substituted or unsubstituted dibenzothiophenyl, a substituted or unsubstituted phenanthrenyl, a substituted or unsubstituted benzophenanthrenyl, a substituted or unsubstituted chrysenyl, a substituted or unsubstituted triphenylenyl, a substituted or unsubstituted fluoranthenyl, or a substituted or unsubstituted benzonapthofuranyl. Wherein, the substituents, for example, may be substituted with at least one of deuterium, cyano, phenyl, and naphthyl.

According to one embodiment, at least one of Ar₇ to Ar₉ in Formula 3 may be represented by any one of the following Formulas 2-2, 3-1, and 3-2.

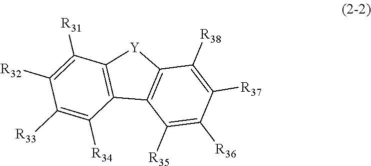

In Formula 2-2,
Y represents —O—, —S—, or —NR₃₉;
R₃₉ represents a substituted or unsubstituted (C6-C30)aryl, or a substituted or unsubstituted (3- to 30-membered)heteroaryl; and
R₃₁ to R₃₈ each independently represent a connection position connected to L₇ to L₉ in Formula 3; or hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to the adjacent substituents to form a ring(s).

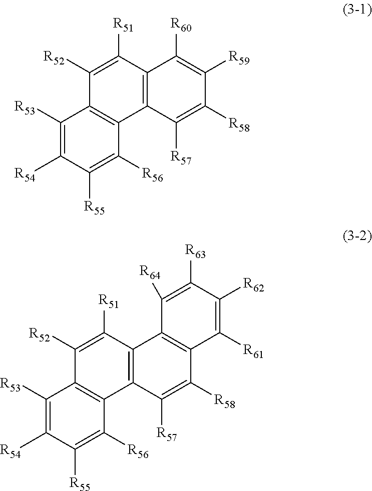

In Formulas 3-1 and 3-2
R₅₁ to R₆₄ each independently represent a connection position connected to L₇ to L₉ in Formula 3; or hydrogen, deuterium, a halogen, a cyano, a substituted or unsubstituted (C1-C30)alkyl, a substituted or unsubstituted (C3-C30)cycloalkyl, a substituted or unsubstituted (3-to 7-membered)heterocycloalkyl, a substituted or unsubstituted fused ring of (C3-C30) aliphatic ring and (C6-C30) aromatic ring, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino.

In Formula 2-2, one of R₃₁ to R₃₈ may be a connection position connected to L₇ to L₉ in Formula 3.

In Formula 3-1, one of R₅₁ to R₆₀ may be a connection position connected to L₇ to L₉ in Formula 3.

In Formula 3-2, one of R₅₁ to R₅₈ and R₆₁ to R₆₄ may be a connection position connected to L₇ to L₉ in Formula 3.

According to one embodiment, the compound represented by Formula 3 may be more specifically illustrated by the following compounds, but is not limited thereto:

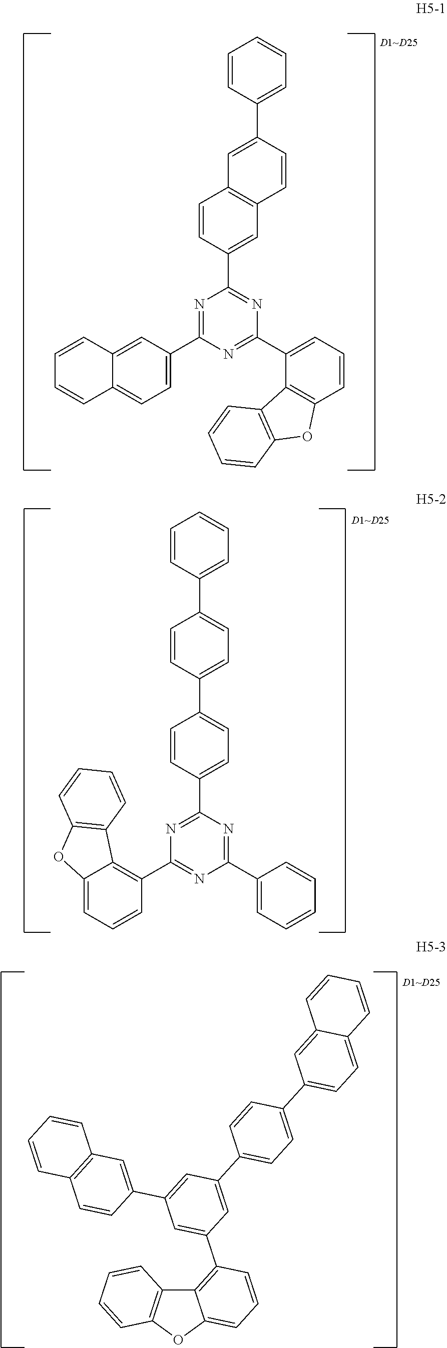

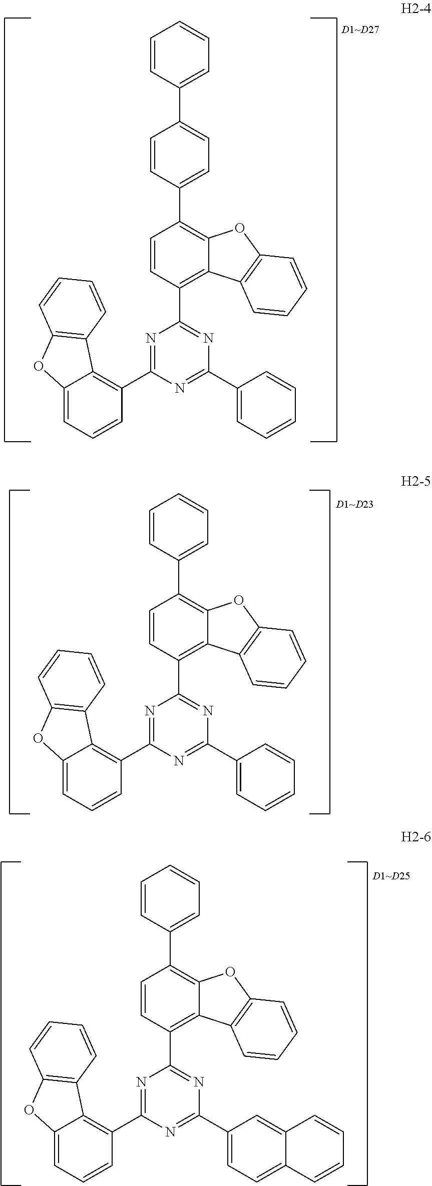

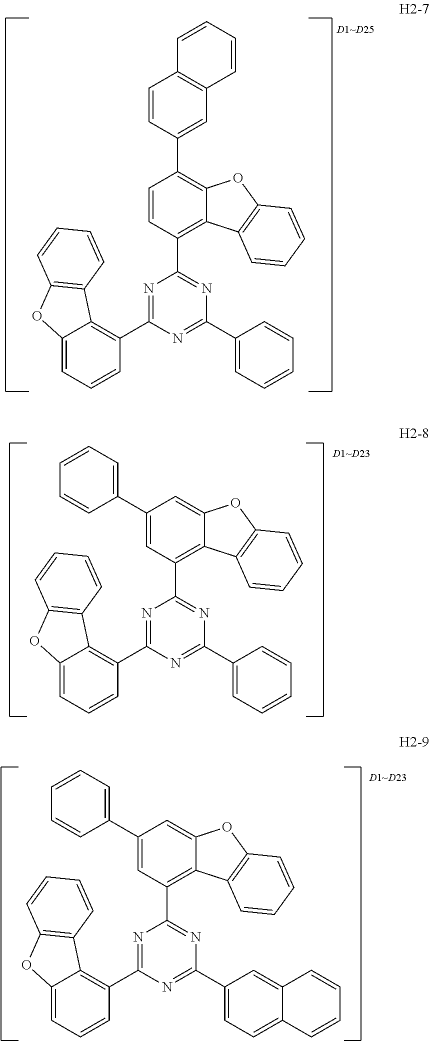

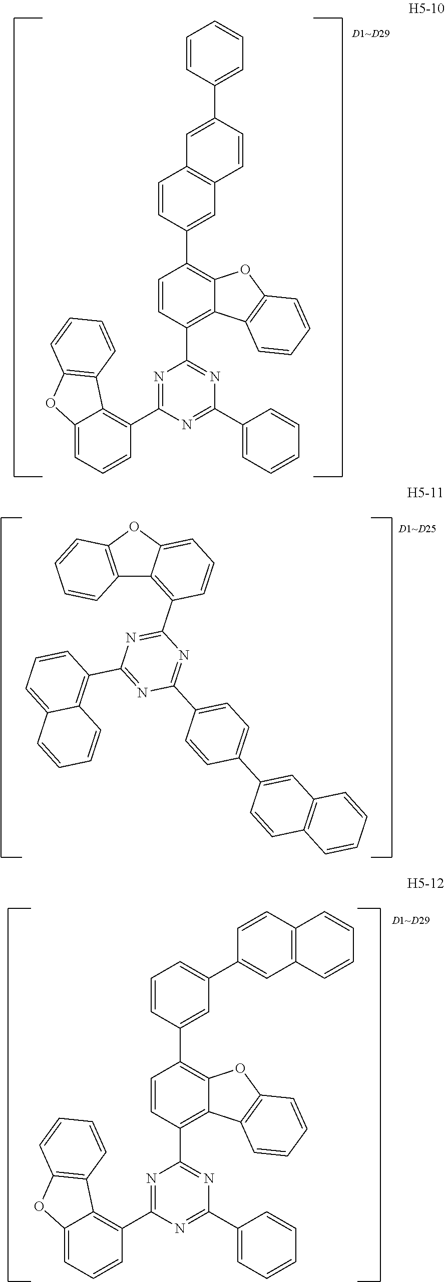

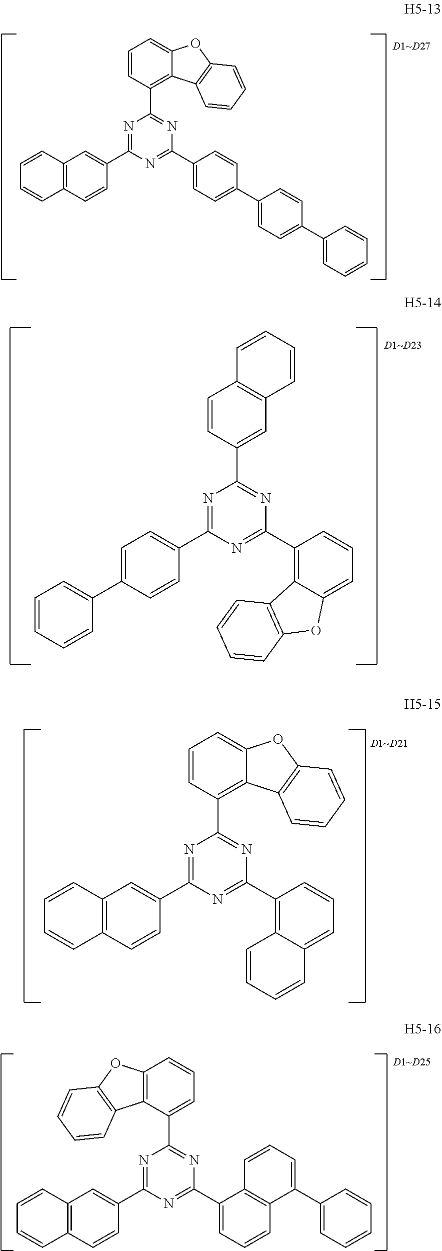

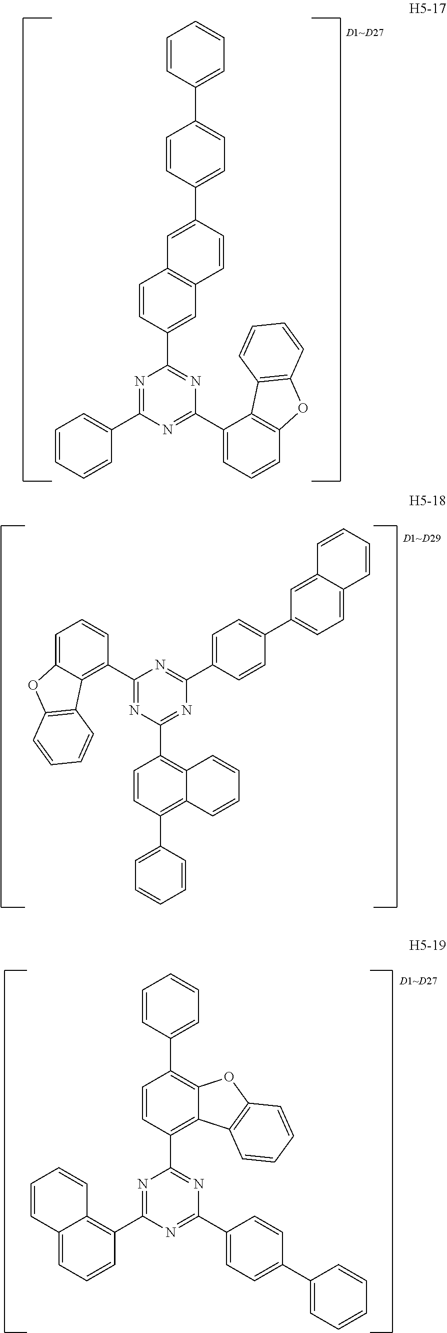

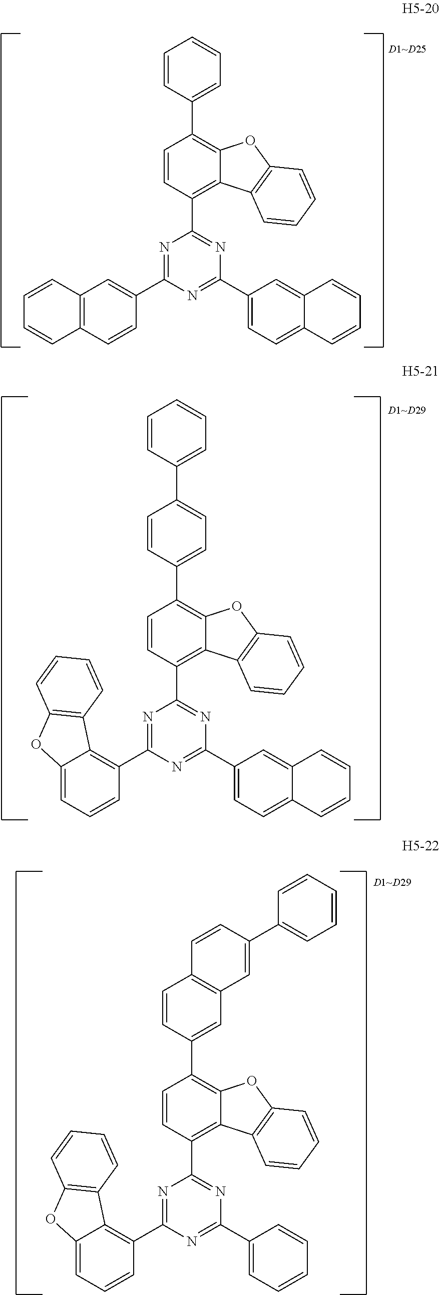

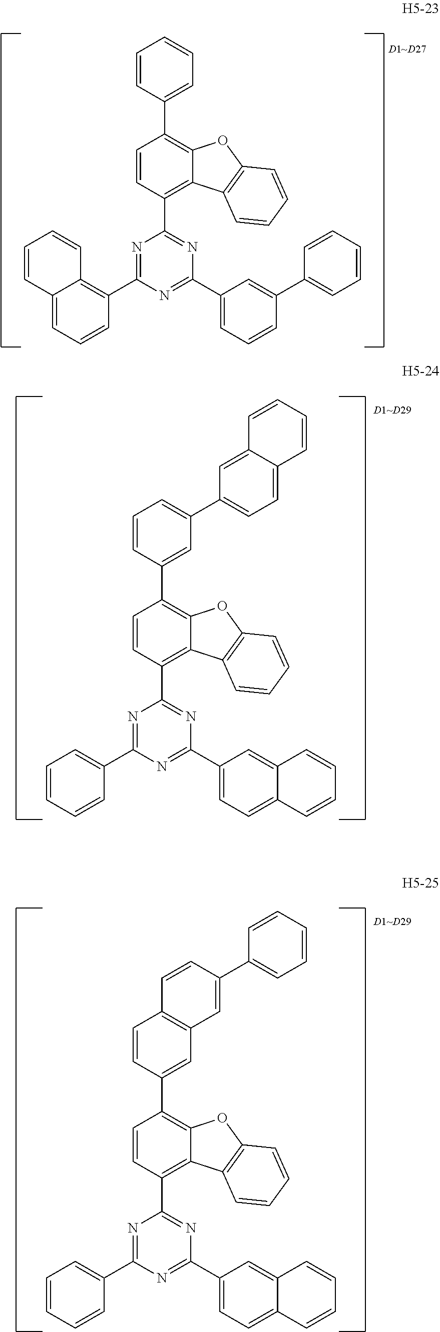

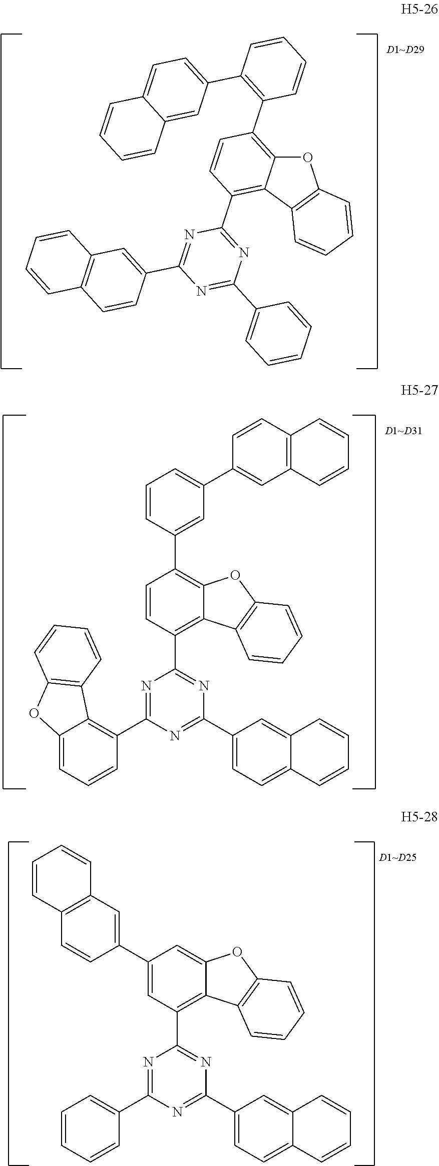

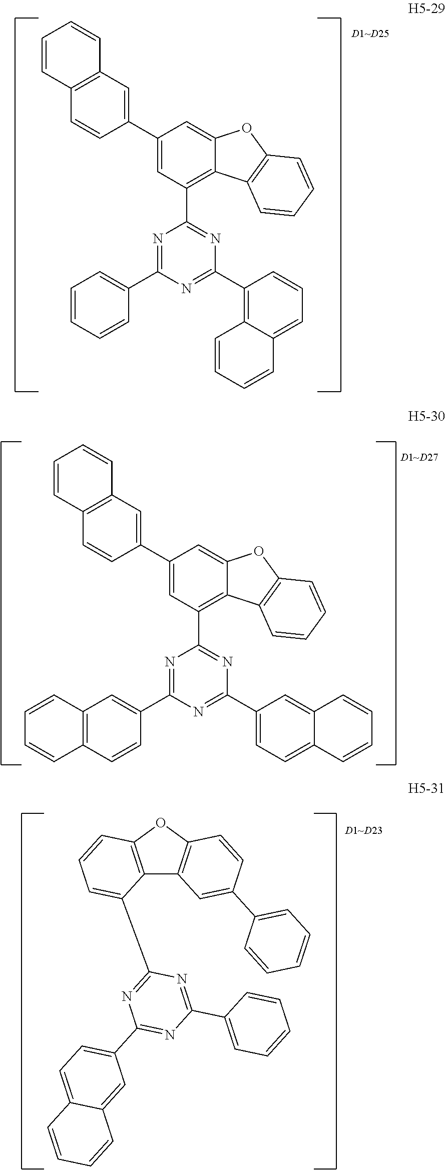

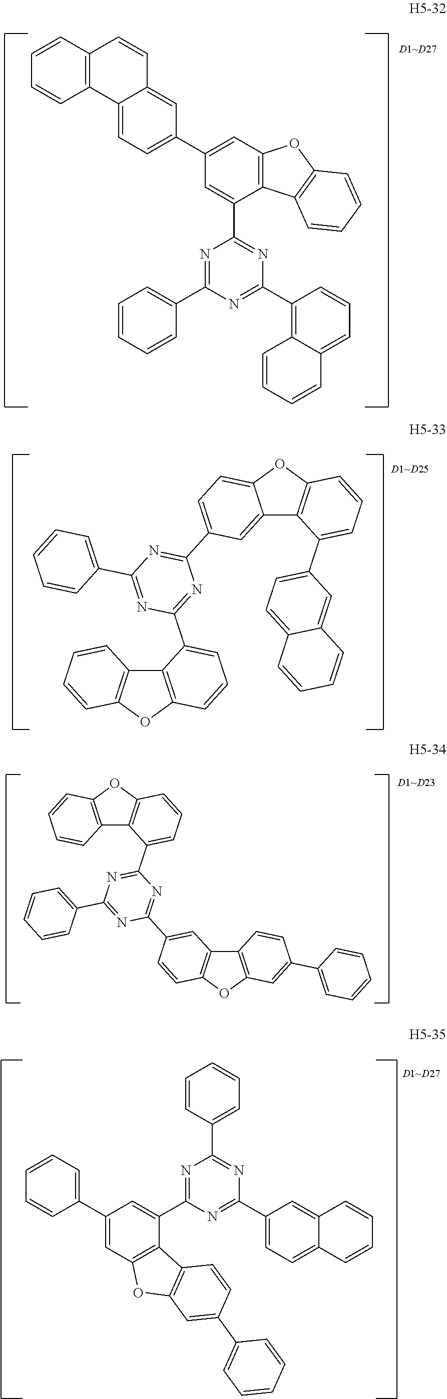

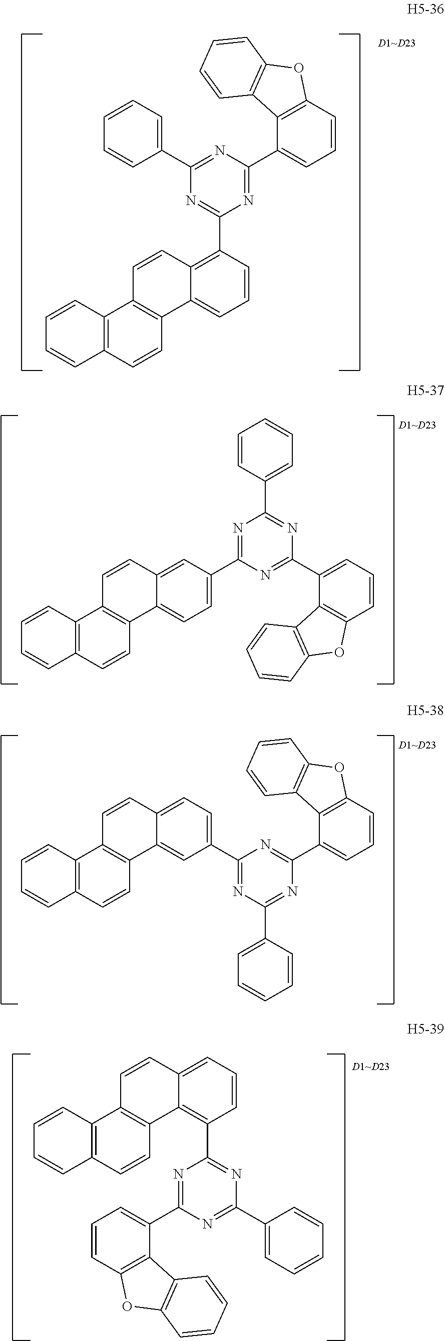

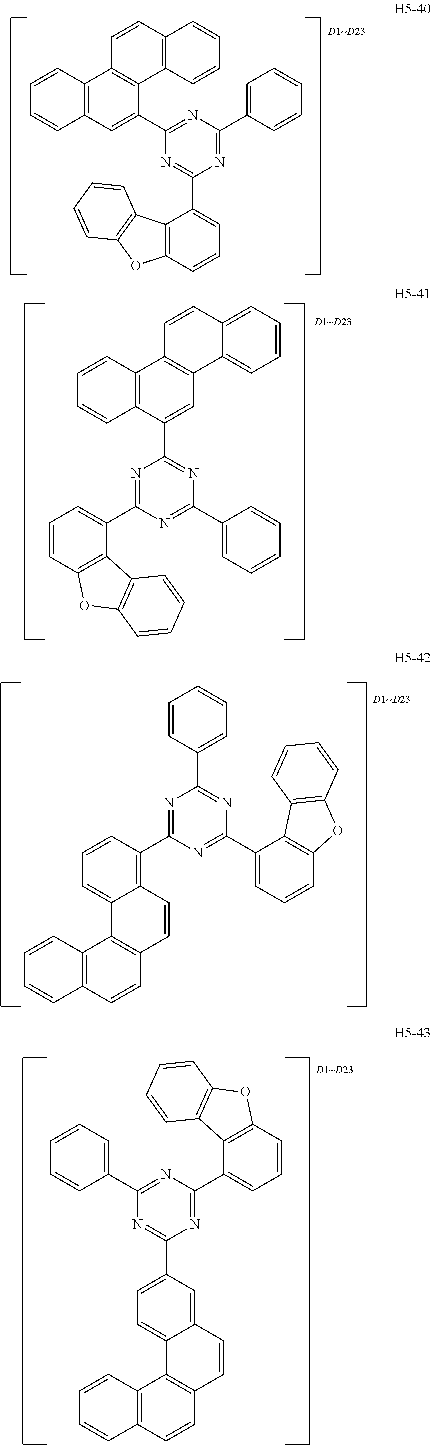

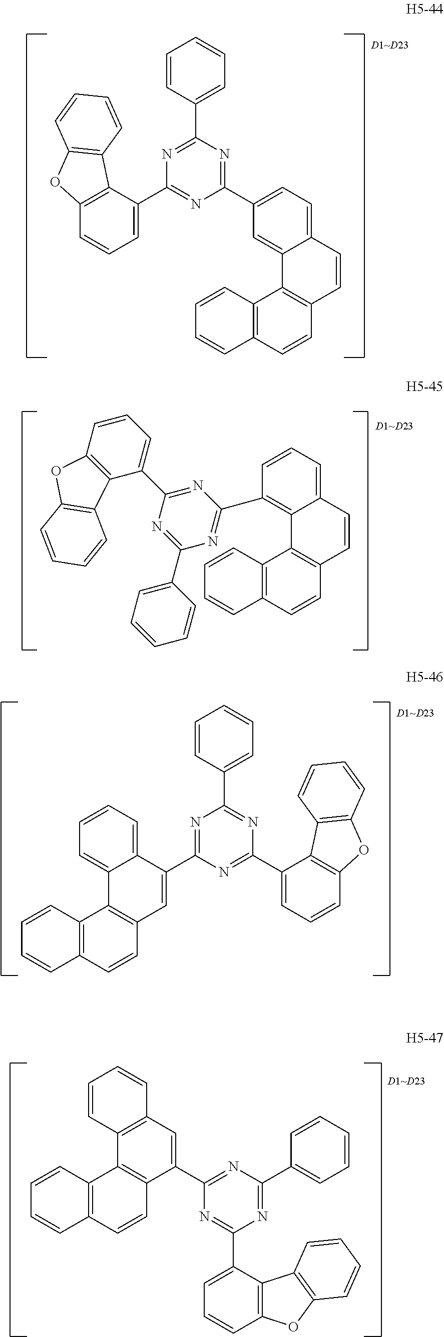

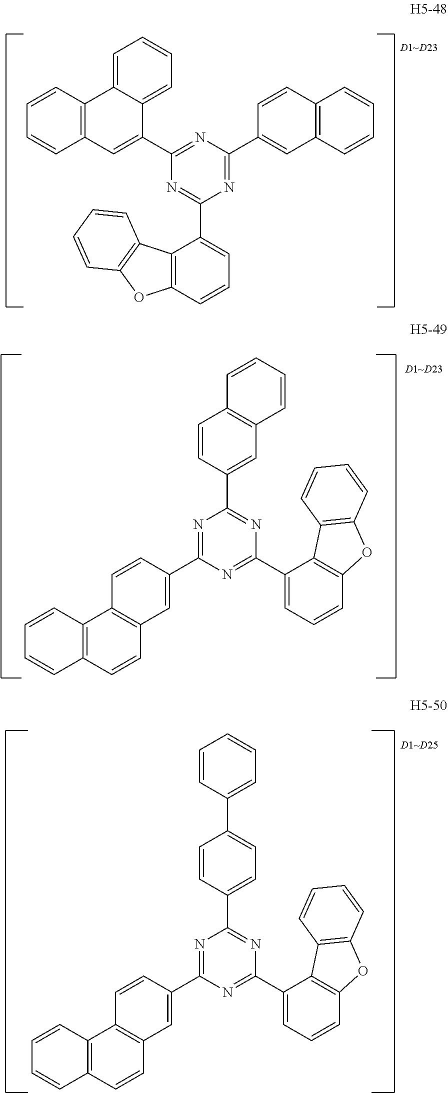

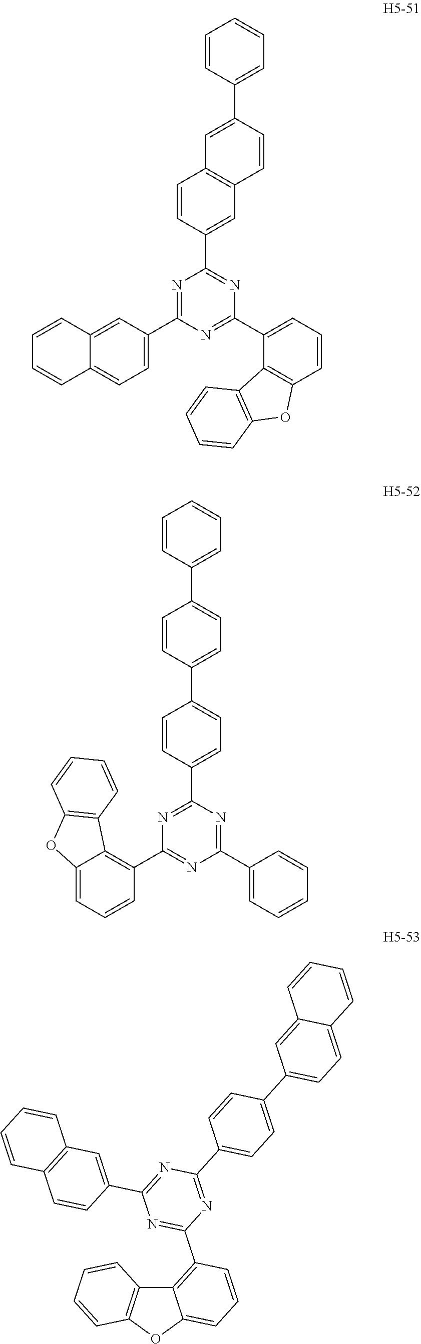 [SMILES: c1ccc(-c2ccc3cc(-c4nc(-c5ccc6ccccc6c5)nc(-c5cccc6oc7ccccc7c56)n4)ccc3c2)cc1 ; c1ccc(-c2ccc(-c3ccc(-c4nc(-c5ccccc5)nc(-c5cccc6oc7ccccc7c56)n4)cc3)cc2)cc1 ; c1ccc2cc(-c3ccc(-c4nc(-c5ccc6ccccc6c5)nc(-c5cccc6oc7ccccc7c56)n4)cc3)ccc2c1]

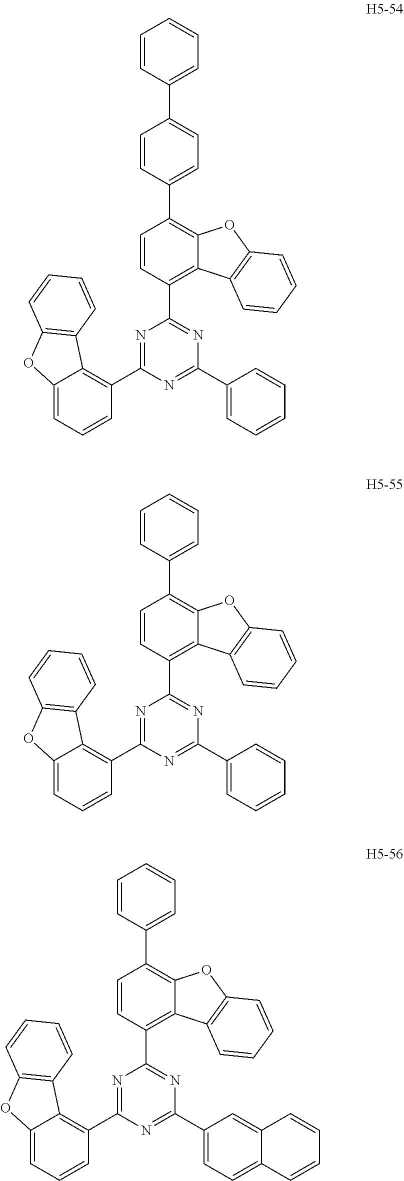 [SMILES: c1ccc(-c2ccc(-c3ccc(-c4nc(-c5ccccc5)nc(-c5cccc6oc7ccccc7c56)n4)c4c3oc3ccccc34)cc2)cc1 ; c1ccc(-c2nc(-c3cccc4oc5ccccc5c34)nc(-c3ccc(-c4ccccc4)c4oc5ccccc5c34)n2)cc1 ; c1ccc(-c2ccc(-c3nc(-c4ccc5ccccc5c4)nc(-c4cccc5oc6ccccc6c45)n3)c3c2oc2ccccc23)cc1]

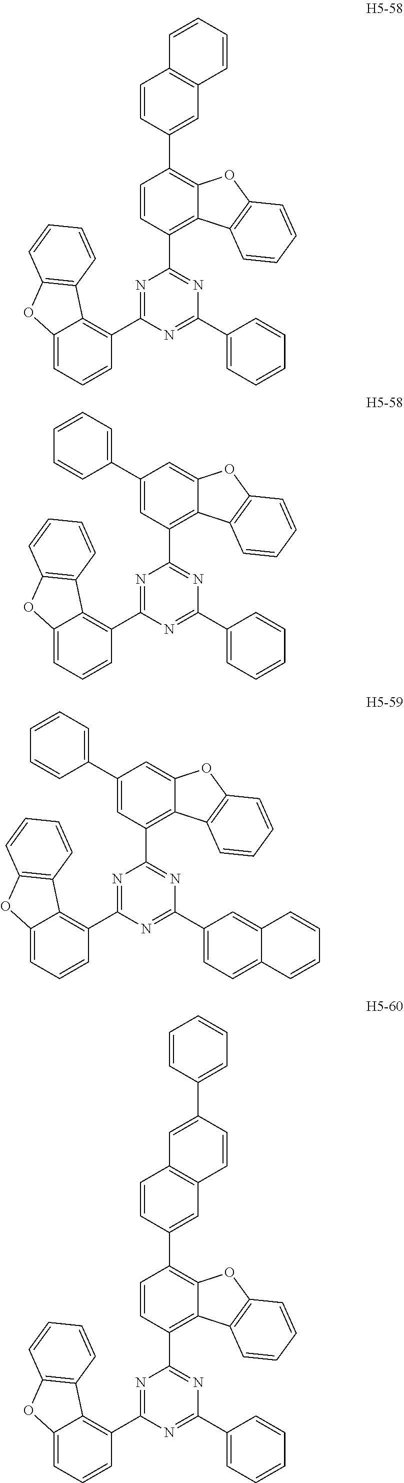

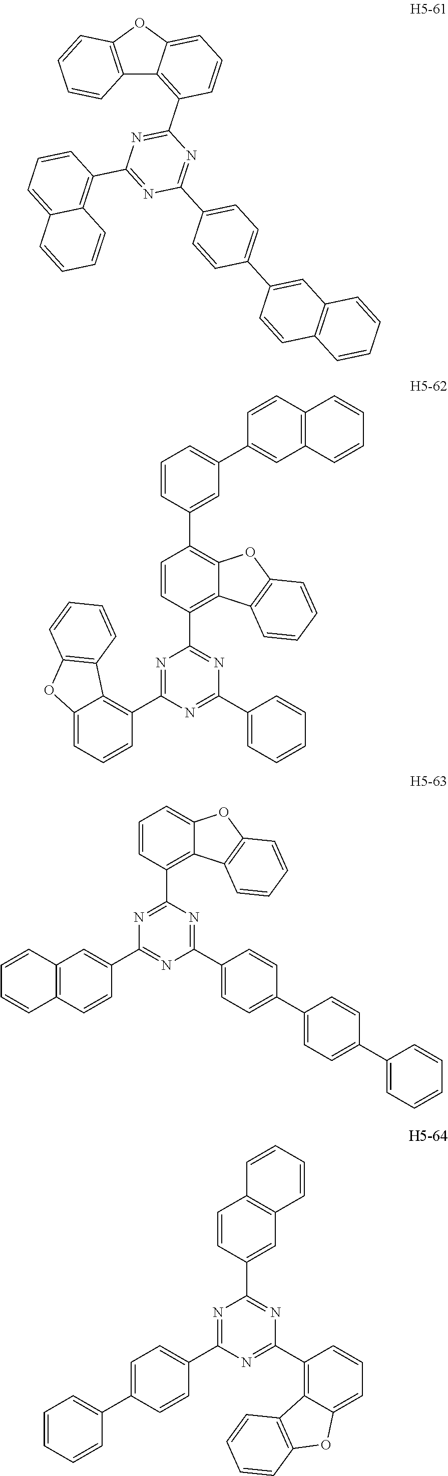

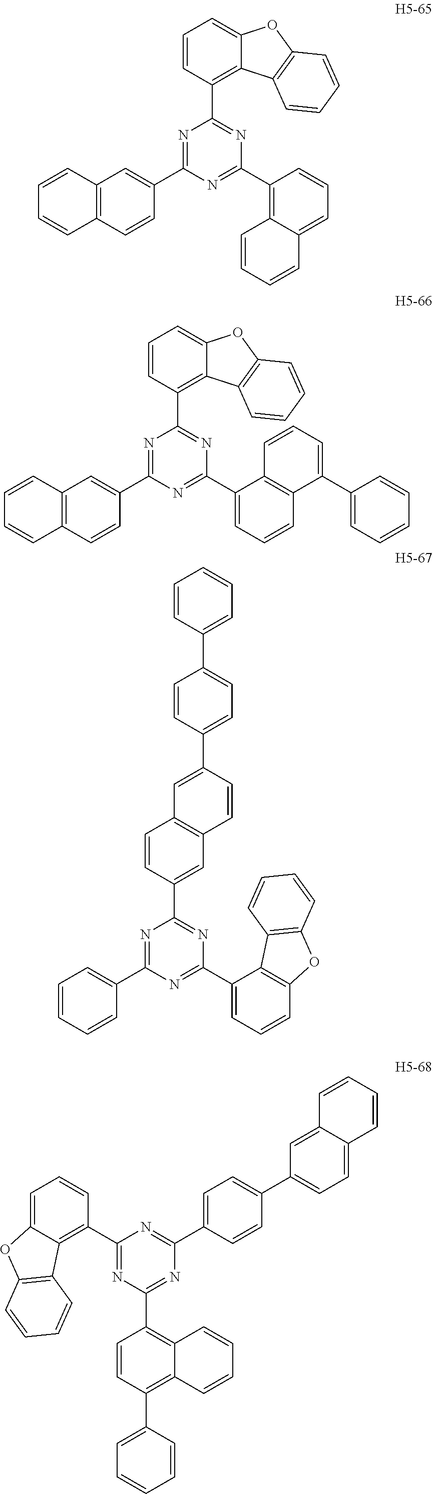 [SMILES: c1ccc2cc(-c3nc(-c4cccc5ccccc45)nc(-c4cccc5oc6ccccc6c45)n3)ccc2c1 ; c1ccc(-c2cccc3c(-c4nc(-c5ccc6ccccc6c5)nc(-c5cccc6oc7ccccc7c56)n4)cccc23)cc1 ; c1ccc(-c2ccc(-c3ccc4cc(-c5nc(-c6ccccc6)nc(-c6cccc7oc8ccccc8c67)n5)ccc4c3)cc2)cc1 ; c1ccc(-c2ccc(-c3nc(-c4ccc(-c5ccc6ccccc6c5)cc4)nc(-c4cccc5oc6ccccc6c45)n3)c3ccccc23)cc1]

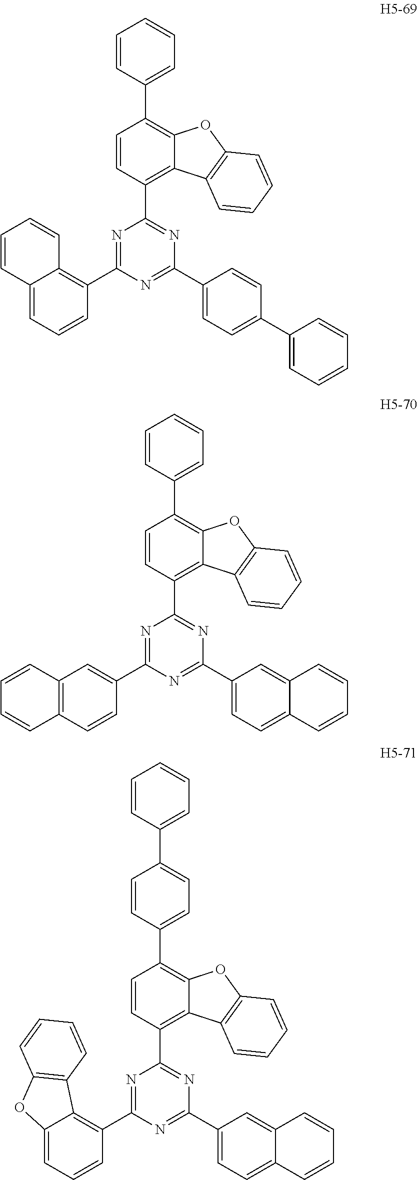 [SMILES: c1ccc(-c2ccc(-c3nc(-c4cccc5ccccc45)nc(-c4ccc(-c5ccccc5)c5oc6ccccc6c45)n3)cc2)cc1 ; c1ccc(-c2ccc(-c3nc(-c4ccc5ccccc5c4)nc(-c4ccc5ccccc5c4)n3)c3c2oc2ccccc23)cc1 ; c1ccc(-c2ccc(-c3ccc(-c4nc(-c5ccc6ccccc6c5)nc(-c5cccc6oc7ccccc7c56)n4)c4c3oc3ccccc34)cc2)cc1]

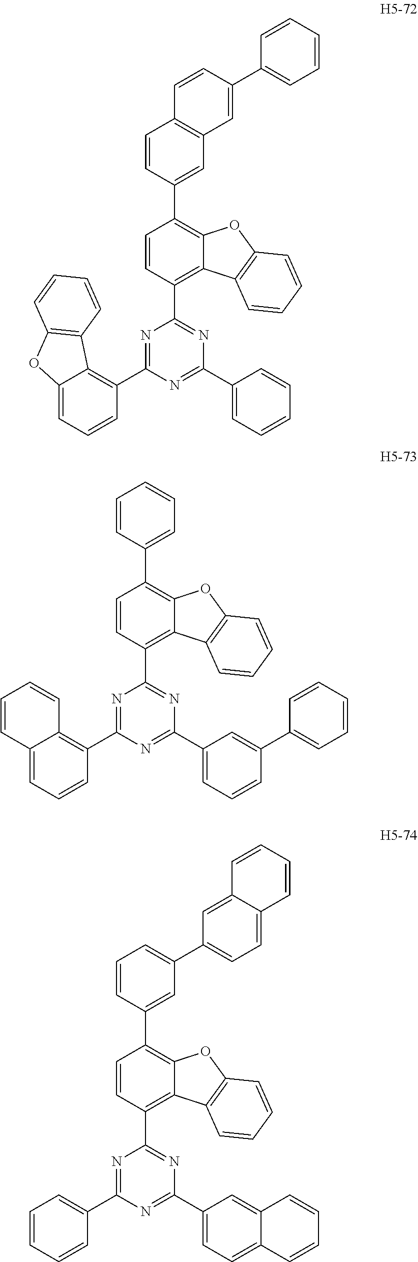 [SMILES: c1ccc(-c2ccc3ccc(-c4ccc(-c5nc(-c6ccccc6)nc(-c6cccc7oc8ccccc8c67)n5)c5c4oc4ccccc45)cc3c2)cc1 ; c1ccc(-c2cccc(-c3nc(-c4cccc5ccccc45)nc(-c4ccc(-c5ccccc5)c5oc6ccccc6c45)n3)c2)cc1 ; c1ccc(-c2nc(-c3ccc4ccccc4c3)nc(-c3ccc(-c4cccc(-c5ccc6ccccc6c5)c4)c4oc5ccccc5c34)n2)cc1]

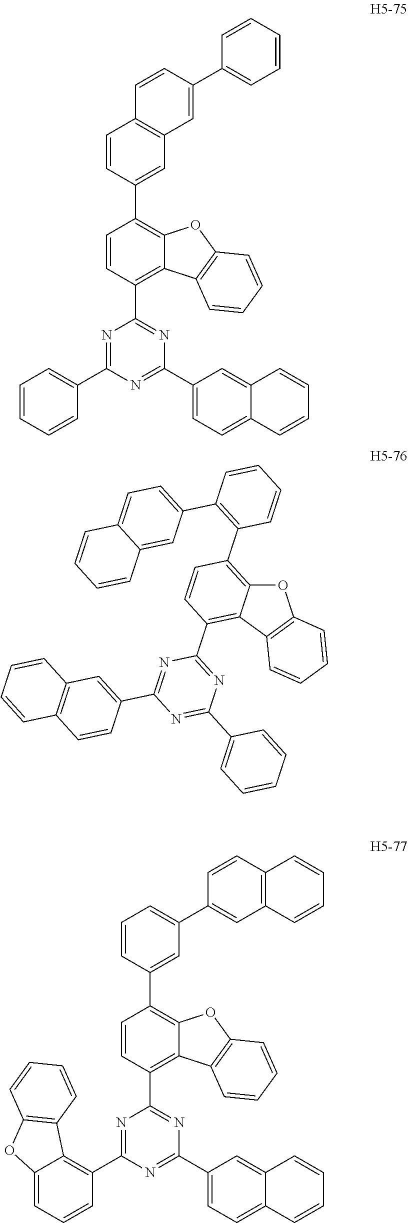

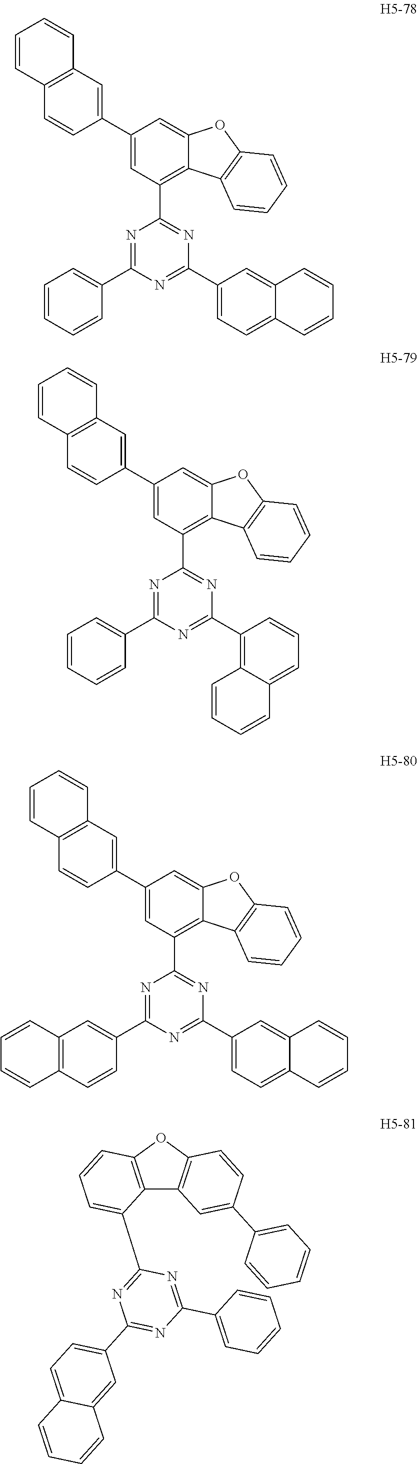

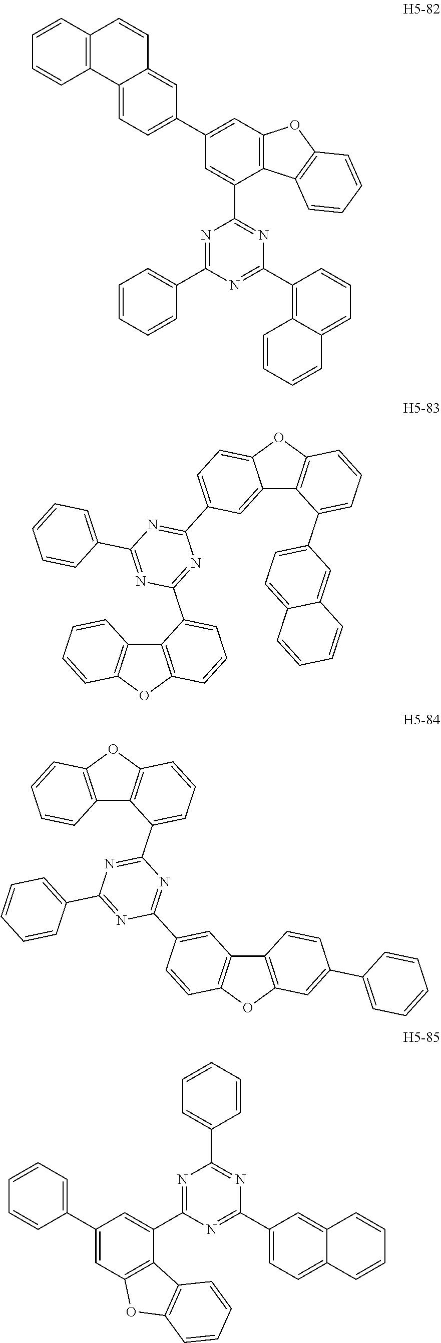 [SMILES: c1ccc(-c2nc(-c3cccc4ccccc34)nc(-c3cc(-c4ccc5c(ccc6ccccc65)c4)cc4oc5ccccc5c34)n2)cc1 ; c1ccc(-c2nc(-c3ccc4oc5cccc(-c6ccc7ccccc7c6)c5c4c3)nc(-c3cccc4oc5ccccc5c34)n2)cc1 ; c1ccc(-c2ccc3c(c2)oc2ccc(-c4nc(-c5ccccc5)nc(-c5cccc6oc7ccccc7c56)n4)cc23)cc1 ; c1ccc(-c2cc(-c3nc(-c4ccccc4)nc(-c4ccc5ccccc5c4)n3)c3c(c2)oc2ccccc23)cc1]

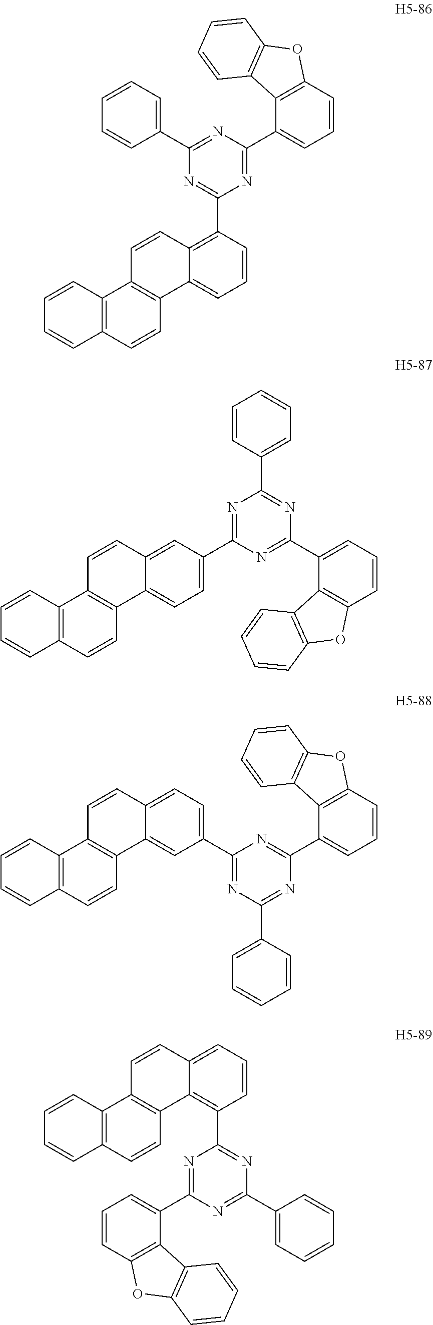 [SMILES: c1ccc(-c2nc(-c3cccc4c3ccc3c5ccccc5ccc43)nc(-c3cccc4oc5ccccc5c34)n2)cc1 ; c1ccc(-c2nc(-c3ccc4c(ccc5c6ccccc6ccc45)c3)nc(-c3cccc4oc5ccccc5c34)n2)cc1 ; c1ccc(-c2nc(-c3ccc4ccc5c6ccccc6ccc5c4c3)nc(-c3cccc4oc5ccccc5c34)n2)cc1 ; c1ccc(-c2nc(-c3cccc4ccc5c6ccccc6ccc5c34)nc(-c3cccc4oc5ccccc5c34)n2)cc1]

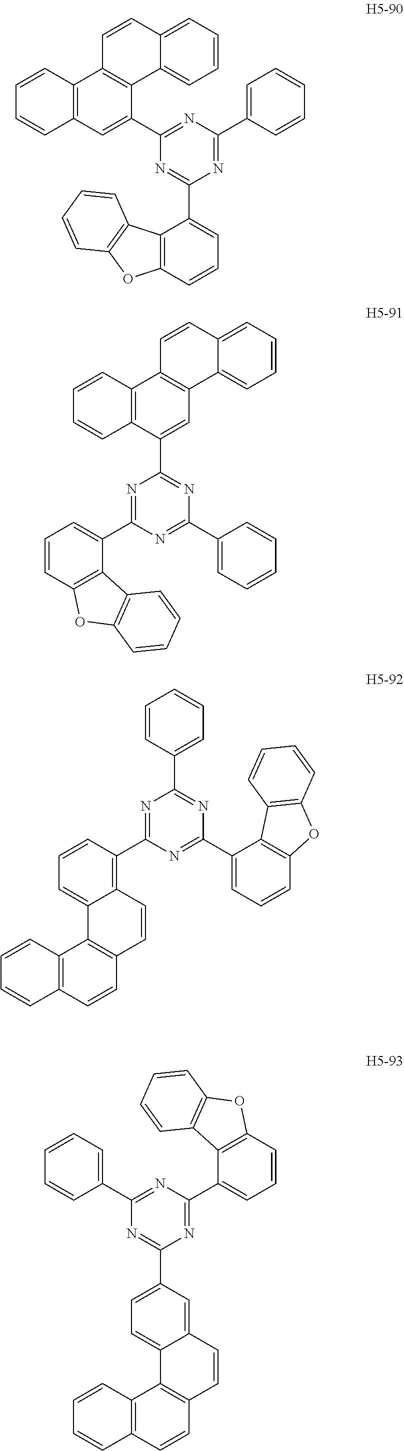

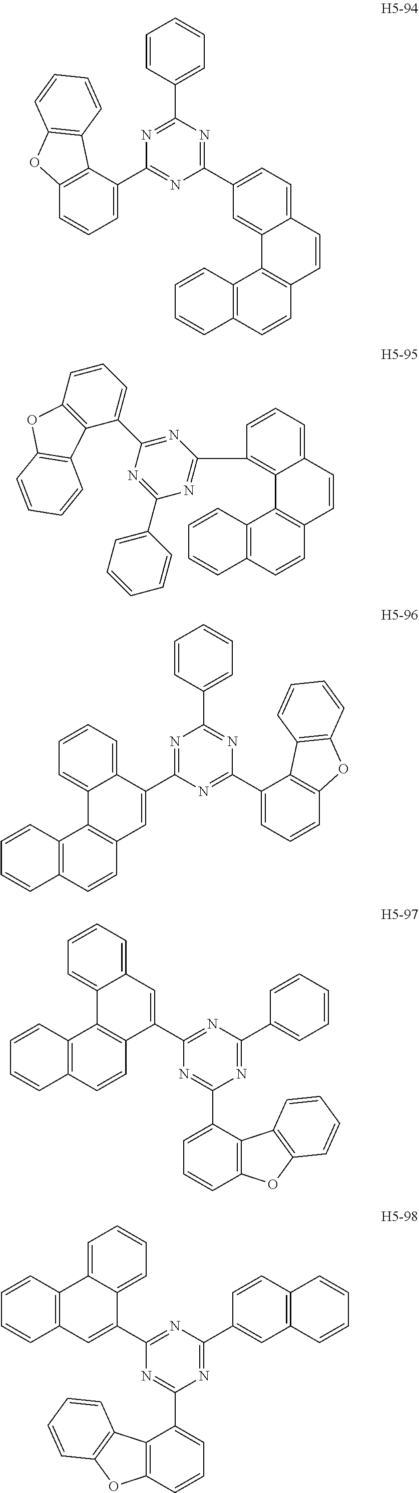

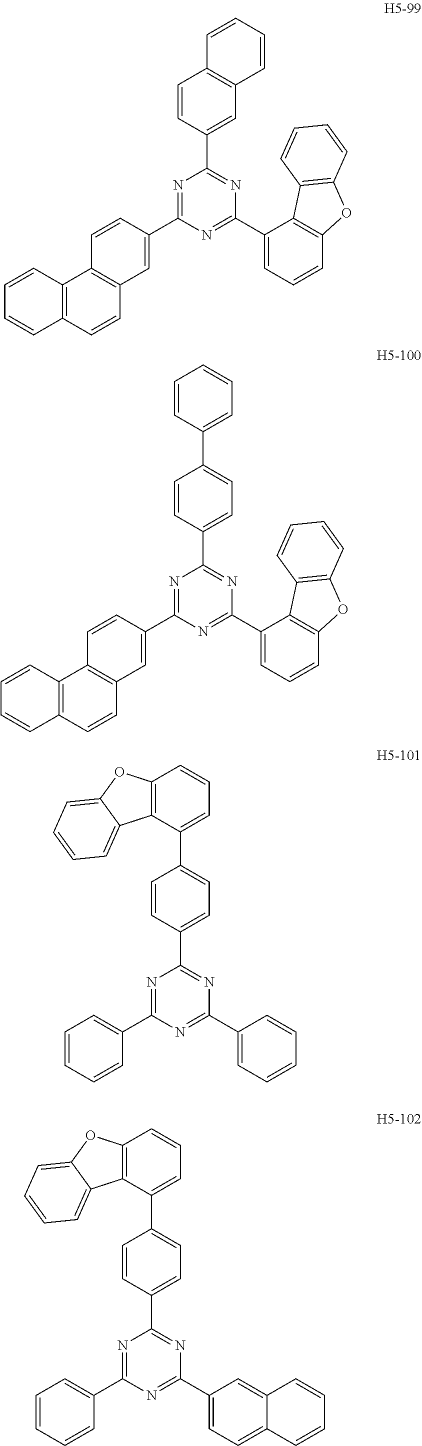 [SMILES: c1ccc2cc(-c3nc(-c4ccc5c(ccc6ccccc65)c4)nc(-c4cccc5oc6ccccc6c45)n3)ccc2c1 ; c1ccc(-c2ccc(-c3nc(-c4ccc5c(ccc6ccccc65)c4)nc(-c4cccc5oc6ccccc6c45)n3)cc2)cc1 ; c1ccc(-c2nc(-c3ccccc3)nc(-c3ccc(-c4cccc5oc6ccccc6c45)cc3)n2)cc1 ; c1ccc(-c2nc(-c3ccc(-c4cccc5oc6ccccc6c45)cc3)nc(-c3ccc4ccccc4c3)n2)cc1]

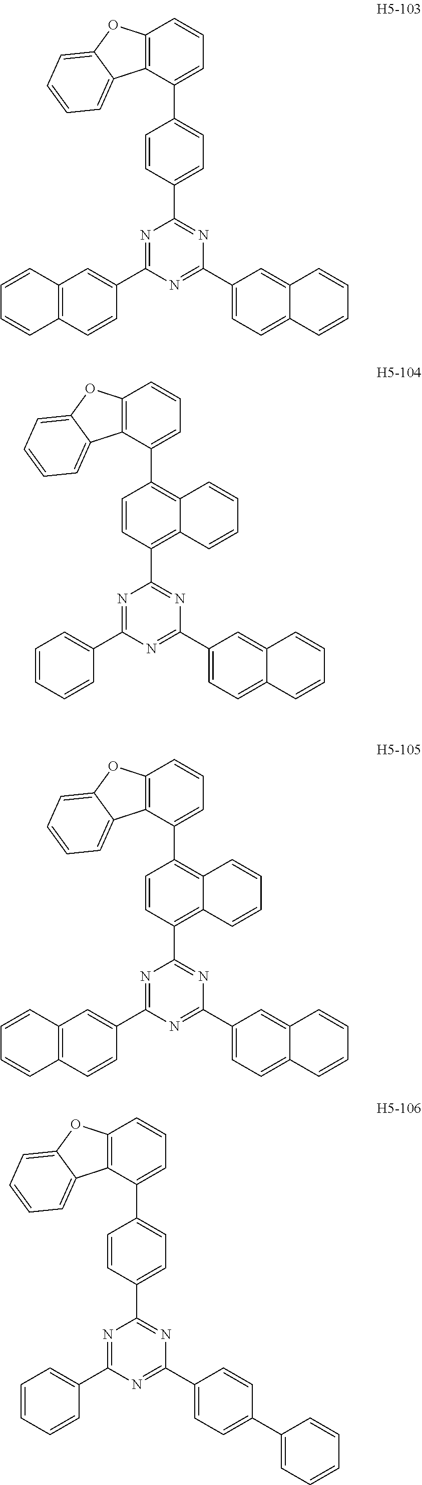 [SMILES: c1ccc2cc(-c3nc(-c4ccc(-c5cccc6oc7ccccc7c56)cc4)nc(-c4ccc5ccccc5c4)n3)ccc2c1 ; c1ccc(-c2nc(-c3ccc4ccccc4c3)nc(-c3ccc(-c4cccc5oc6ccccc6c45)c4ccccc34)n2)cc1 ; c1ccc2cc(-c3nc(-c4ccc5ccccc5c4)nc(-c4ccc(-c5cccc6oc7ccccc7c56)c5ccccc45)n3)ccc2c1 ; c1ccc(-c2ccc(-c3nc(-c4ccccc4)nc(-c4ccc(-c5cccc6oc7ccccc7c56)cc4)n3)cc2)cc1]

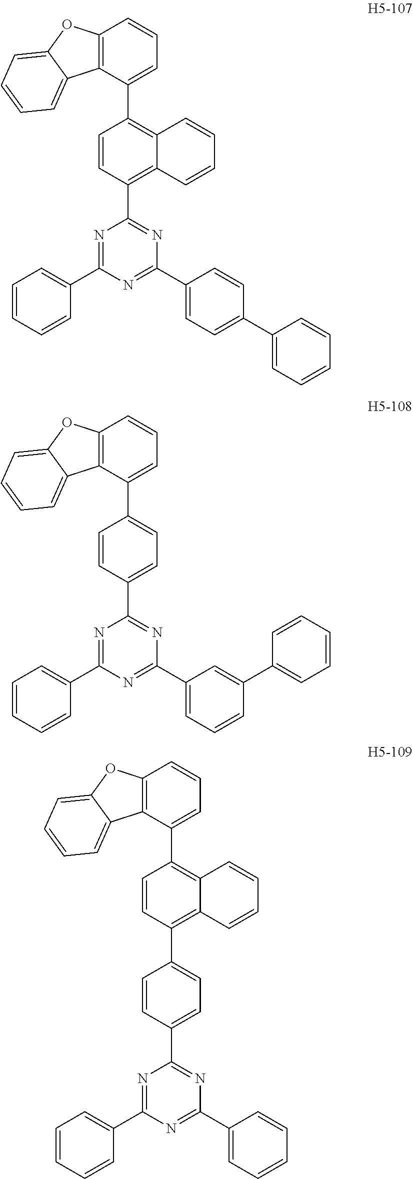 [SMILES: c1ccc(-c2ccc(-c3nc(-c4ccccc4)nc(-c4ccc(-c5cccc6oc7ccccc7c56)c5ccccc45)n3)cc2)cc1 ; c1ccc(-c2cccc(-c3nc(-c4ccccc4)nc(-c4ccc(-c5cccc6oc7ccccc7c56)cc4)n3)c2)cc1 ; c1ccc(-c2nc(-c3ccccc3)nc(-c3ccc(-c4ccc(-c5cccc6oc7ccccc7c56)c5ccccc45)cc3)n2)cc1]

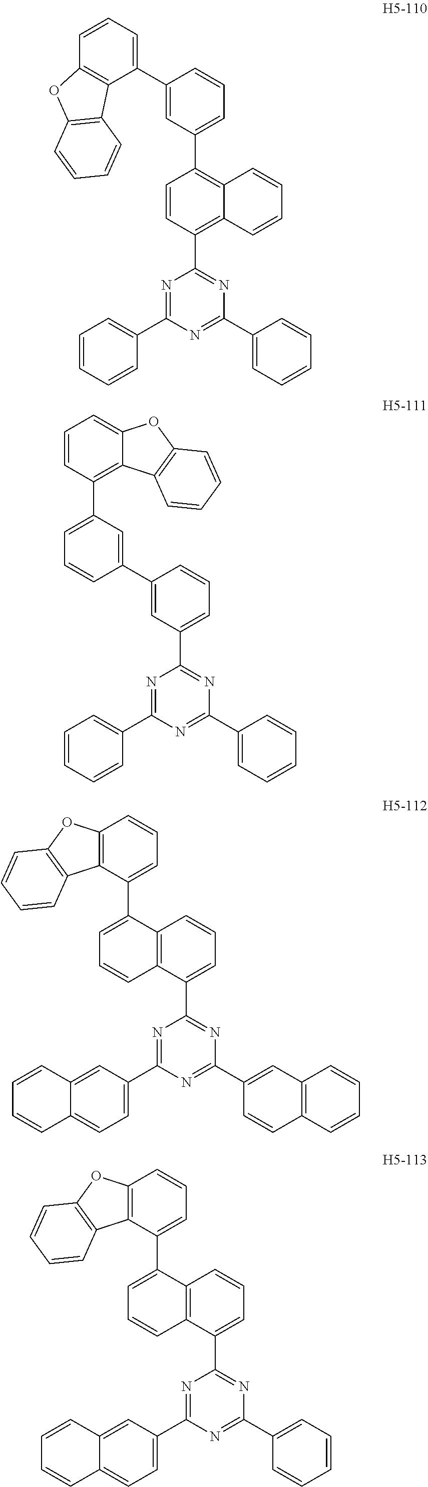

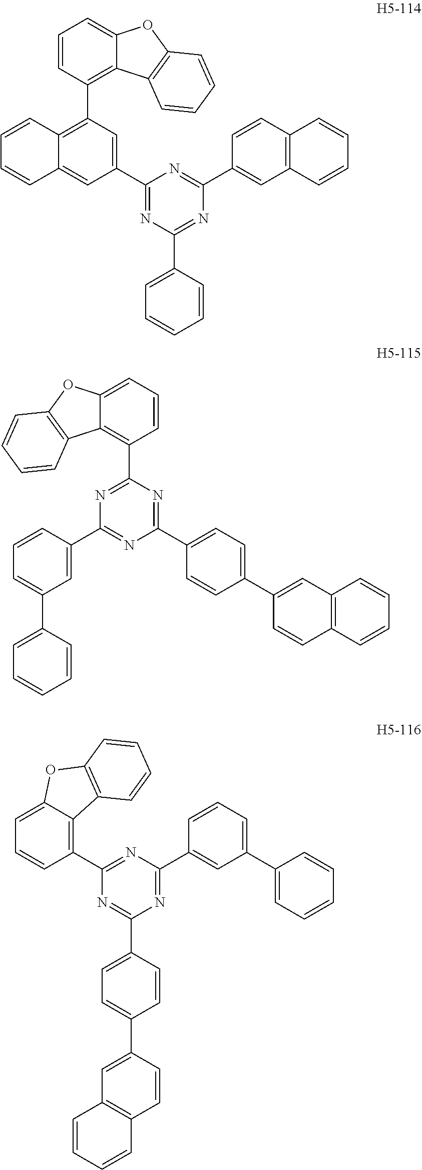 [SMILES: c1ccc(-c2nc(-c3ccc4ccccc4c3)nc(-c3cc(-c4cccc5oc6ccccc6c45)c4ccccc4c3)n2)cc1 ; c1ccc(-c2cccc(-c3nc(-c4ccc(-c5ccc6ccccc6c5)cc4)nc(-c4cccc5oc6ccccc6c45)n3)c2)cc1 ; c1ccc(-c2cccc(-c3nc(-c4ccc(-c5ccc6ccccc6c5)cc4)nc(-c4cccc5oc6ccccc6c45)n3)c2)cc1]

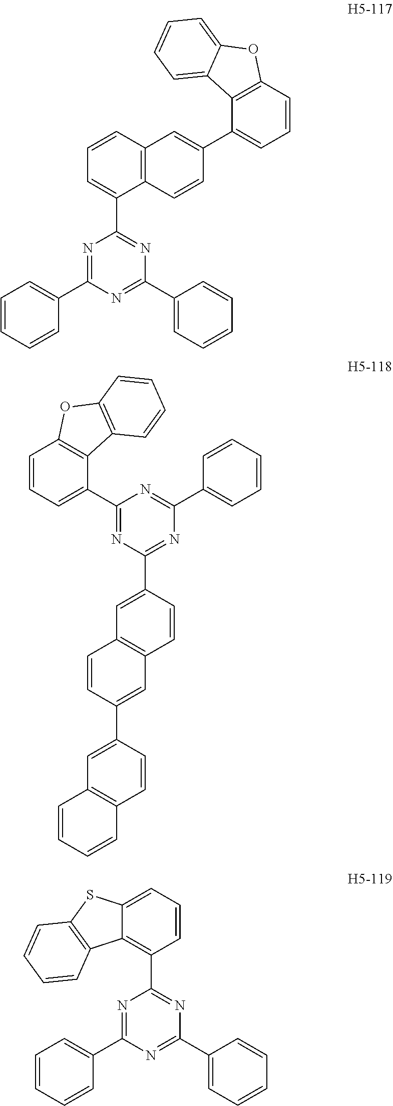

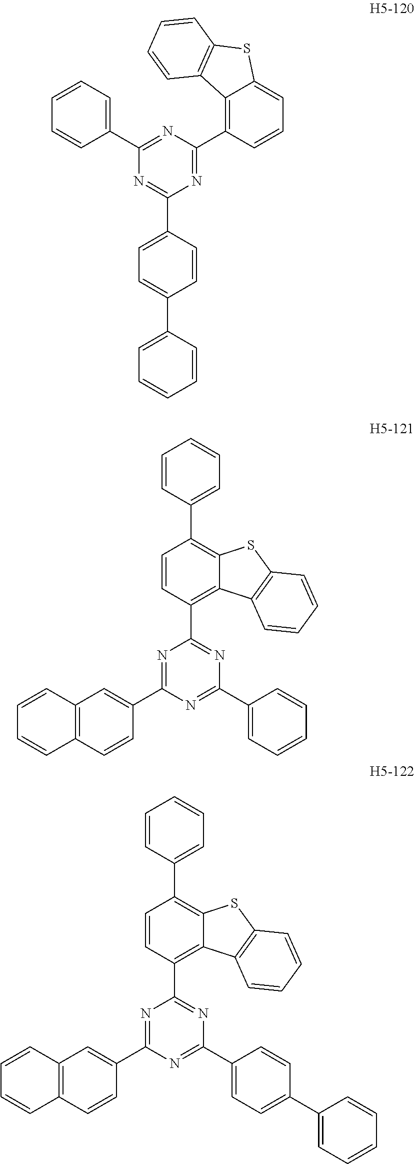

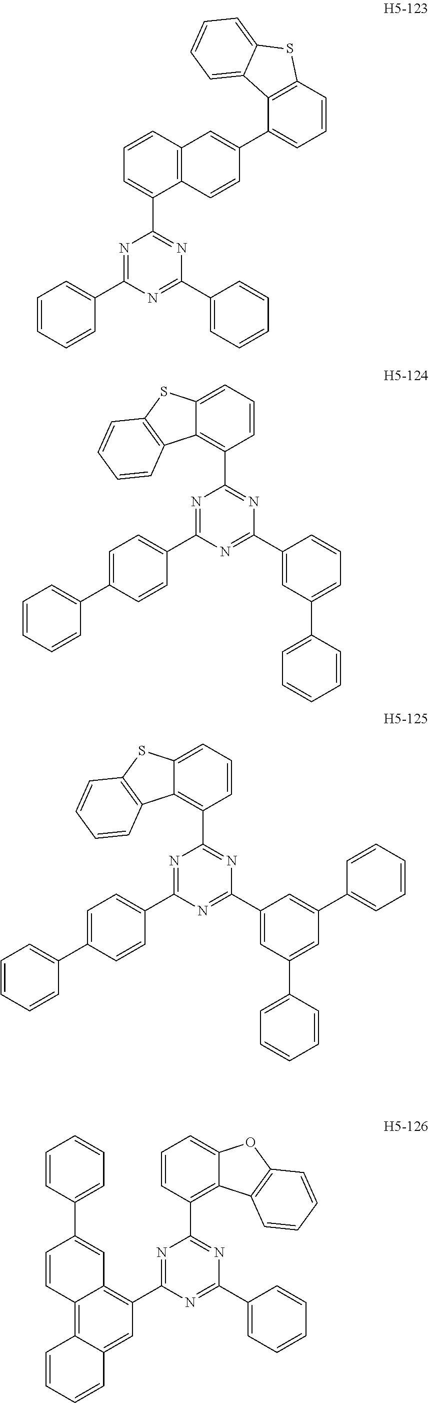

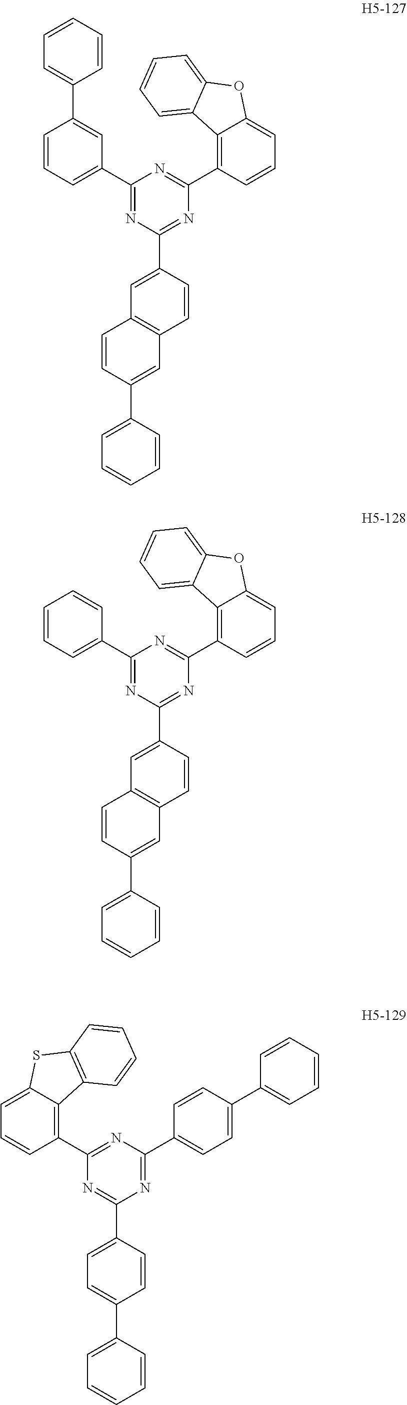 [SMILES: c1ccc(-c2cccc(-c3nc(-c4ccc5cc(-c6ccccc6)ccc5c4)nc(-c4cccc5oc6ccccc6c45)n3)c2)cc1 ; c1ccc(-c2ccc3cc(-c4nc(-c5ccccc5)nc(-c5cccc6oc7ccccc7c56)n4)ccc3c2)cc1 ; c1ccc(-c2ccc(-c3nc(-c4ccc(-c5ccccc5)cc4)nc(-c4cccc5sc6ccccc6c45)n3)cc2)cc1]

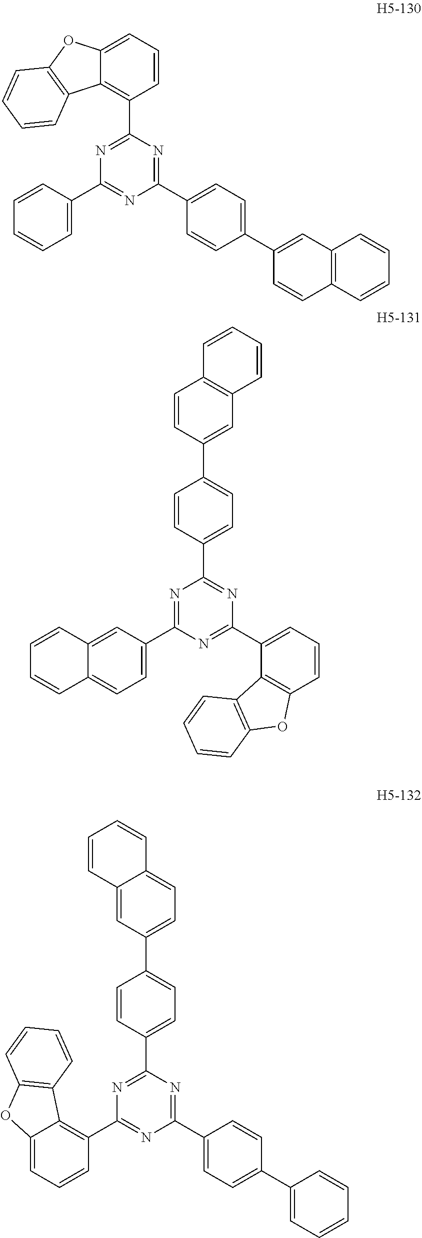 [SMILES: c1ccc(-c2nc(-c3ccc(-c4ccc5ccccc5c4)cc3)nc(-c3cccc4oc5ccccc5c34)n2)cc1 ; c1ccc2cc(-c3ccc(-c4nc(-c5ccc6ccccc6c5)nc(-c5cccc6oc7ccccc7c56)n4)cc3)ccc2c1 ; c1ccc(-c2ccc(-c3nc(-c4ccc(-c5ccc6ccccc6c5)cc4)nc(-c4cccc5oc6ccccc6c45)n3)cc2)cc1]

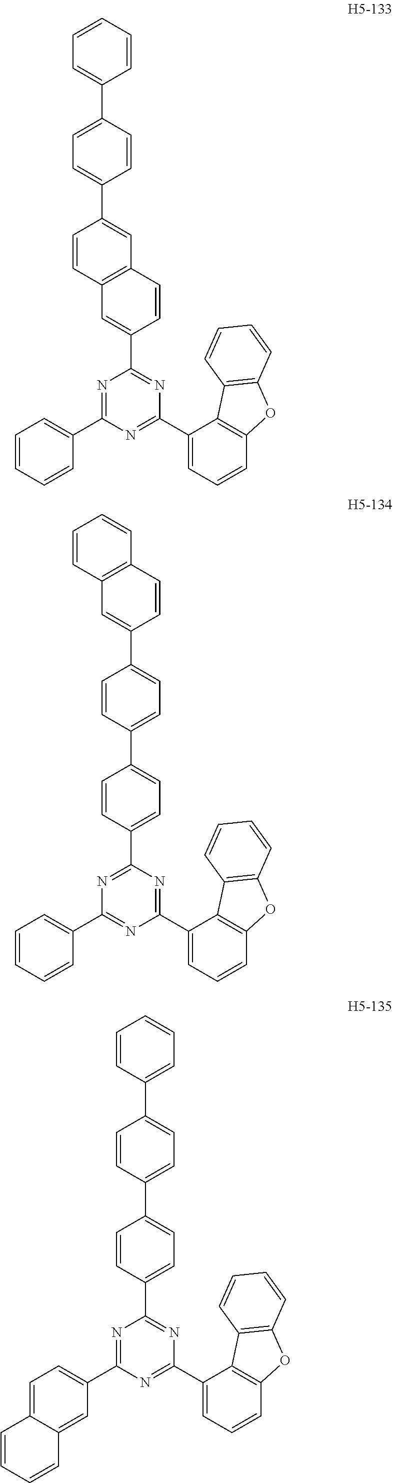 [SMILES: c1ccc(-c2ccc(-c3ccc4cc(-c5nc(-c6ccccc6)nc(-c6cccc7oc8ccccc8c67)n5)ccc4c3)cc2)cc1 ; c1ccc(-c2nc(-c3ccc(-c4ccc(-c5ccc6ccccc6c5)cc4)cc3)nc(-c3cccc4oc5ccccc5c34)n2)cc1 ; c1ccc(-c2ccc(-c3ccc(-c4nc(-c5ccc6ccccc6c5)nc(-c5cccc6oc7ccccc7c56)n4)cc3)cc2)cc1]

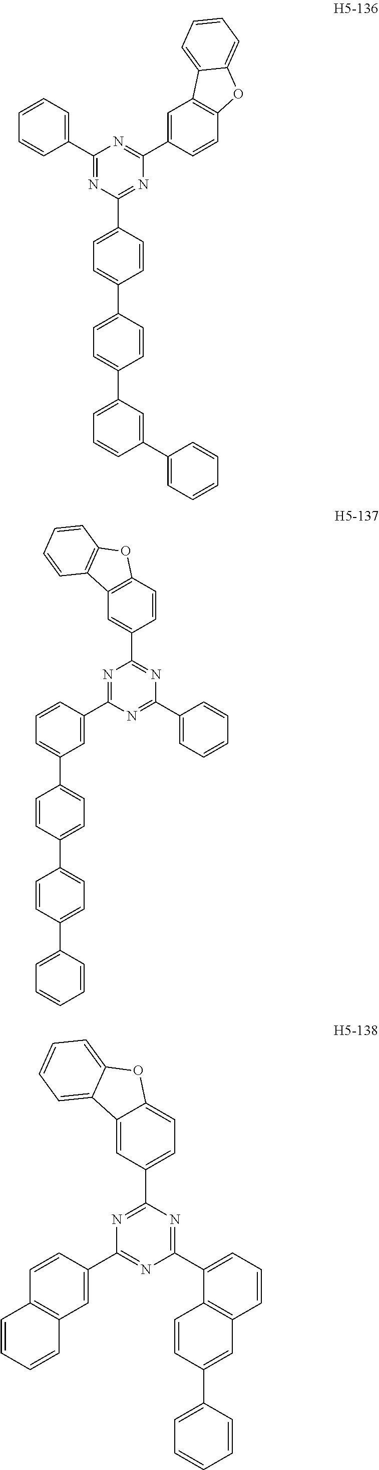

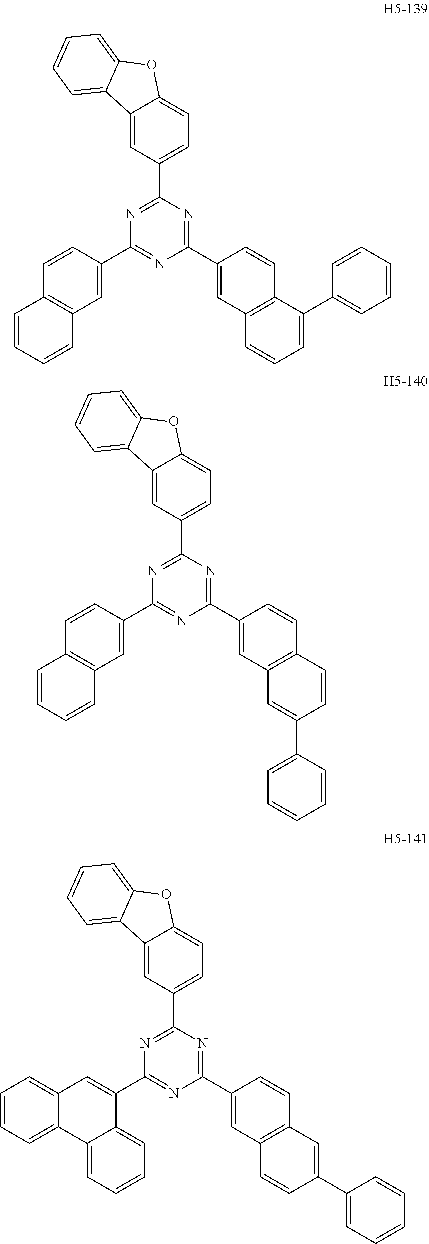 [SMILES: c1ccc(-c2cccc3cc(-c4nc(-c5ccc6ccccc6c5)nc(-c5ccc6oc7ccccc7c6c5)n4)ccc23)cc1 ; c1ccc(-c2ccc3ccc(-c4nc(-c5ccc6ccccc6c5)nc(-c5ccc6oc7ccccc7c6c5)n4)cc3c2)cc1 ; c1ccc(-c2ccc3cc(-c4nc(-c5ccc6oc7ccccc7c6c5)nc(-c5cc6ccccc6c6ccccc56)n4)ccc3c2)cc1]

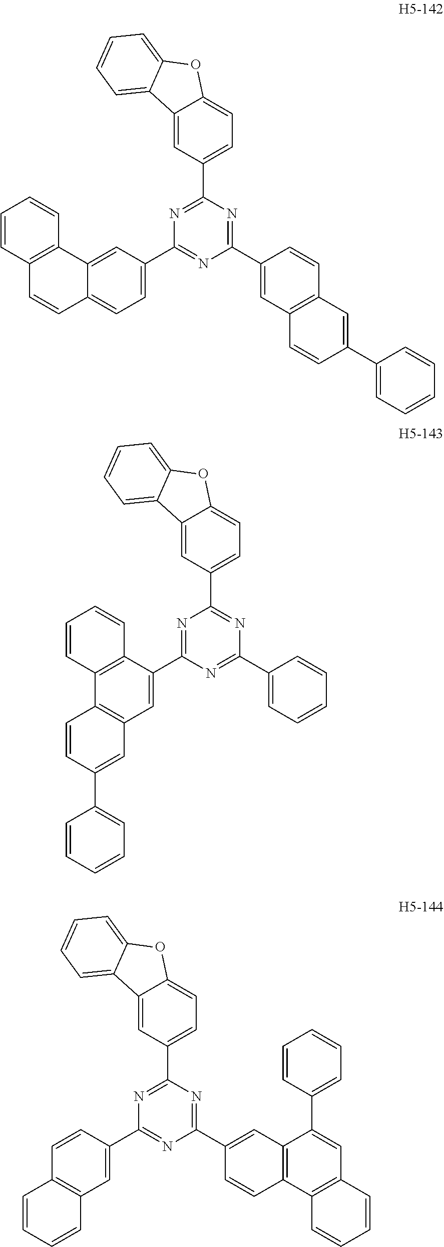

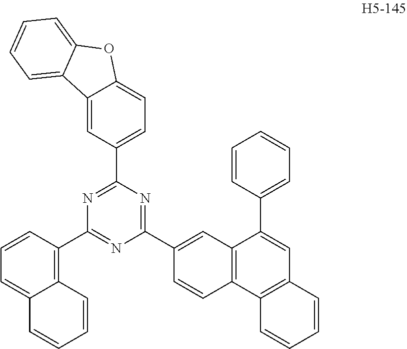 [SMILES: c1ccc(-c2cc3ccccc3c3ccc(-c4nc(-c5ccc6oc7ccccc7c6c5)nc(-c5cccc6ccccc56)n4)cc23)cc1]

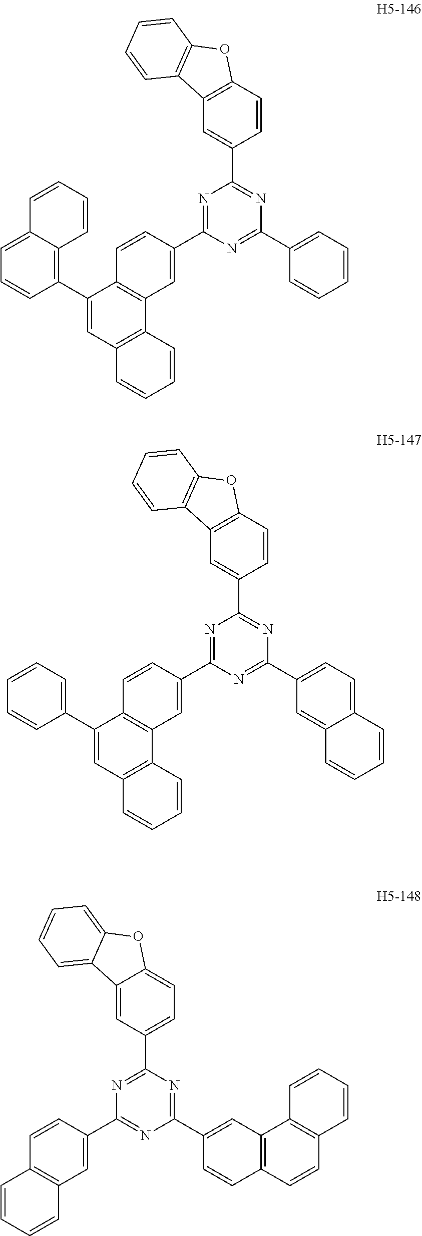 [SMILES: c1ccc(-c2nc(-c3ccc4oc5ccccc5c4c3)nc(-c3ccc4c(-c5cccc6ccccc56)cc5ccccc5c4c3)n2)cc1 ; c1ccc(-c2cc3ccccc3c3cc(-c4nc(-c5ccc6ccccc6c5)nc(-c5ccc6oc7ccccc7c6c5)n4)ccc23)cc1 ; c1ccc2cc(-c3nc(-c4ccc5ccc6ccccc6c5c4)nc(-c4ccc5oc6ccccc6c5c4)n3)ccc2c1]

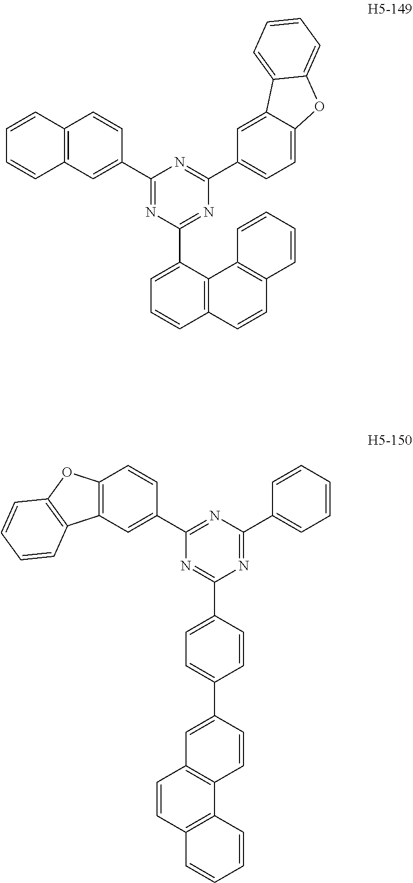 [SMILES: c1ccc2cc(-c3nc(-c4ccc5oc6ccccc6c5c4)nc(-c4cccc5ccc6ccccc6c45)n3)ccc2c1 ; c1ccc(-c2nc(-c3ccc(-c4ccc5c(ccc6ccccc65)c4)cc3)nc(-c3ccc4oc5ccccc5c4c3)n2)cc1]

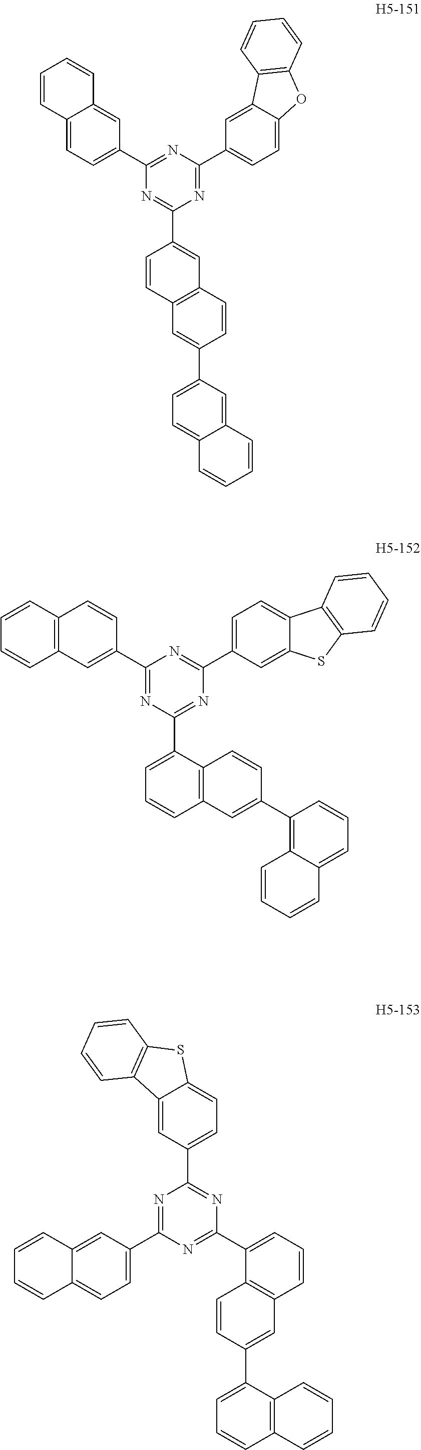 [SMILES: c1ccc2cc(-c3ccc4cc(-c5nc(-c6ccc7ccccc7c6)nc(-c6ccc7oc8ccccc8c7c6)n5)ccc4c3)ccc2c1 ; c1ccc2cc(-c3nc(-c4ccc5c(c4)sc4ccccc45)nc(-c4cccc5cc(-c6cccc7ccccc67)ccc45)n3)ccc2c1 ; c1ccc2cc(-c3nc(-c4ccc5sc6ccccc6c5c4)nc(-c4cccc5cc(-c6cccc7ccccc67)ccc45)n3)ccc2c1]

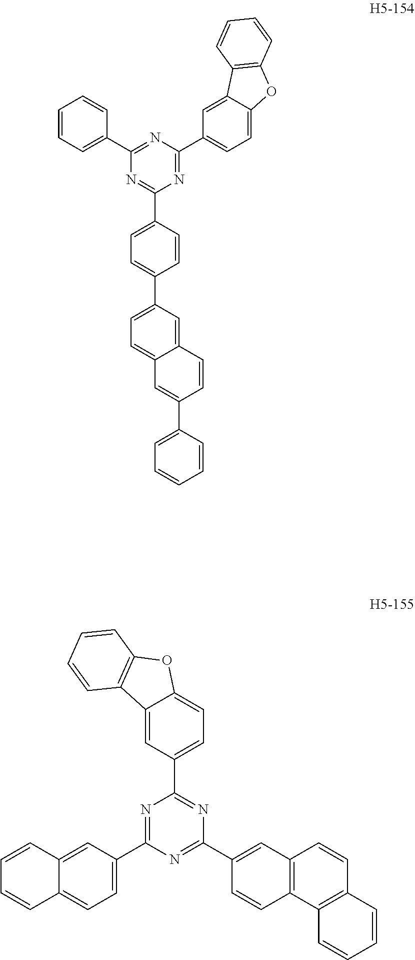 [SMILES: c1ccc(-c2ccc3cc(-c4ccc(-c5nc(-c6ccccc6)nc(-c6ccc7oc8ccccc8c7c6)n5)cc4)ccc3c2)cc1 ; c1ccc2cc(-c3nc(-c4ccc5c(ccc6ccccc65)c4)nc(-c4ccc5oc6ccccc6c5c4)n3)ccc2c1]

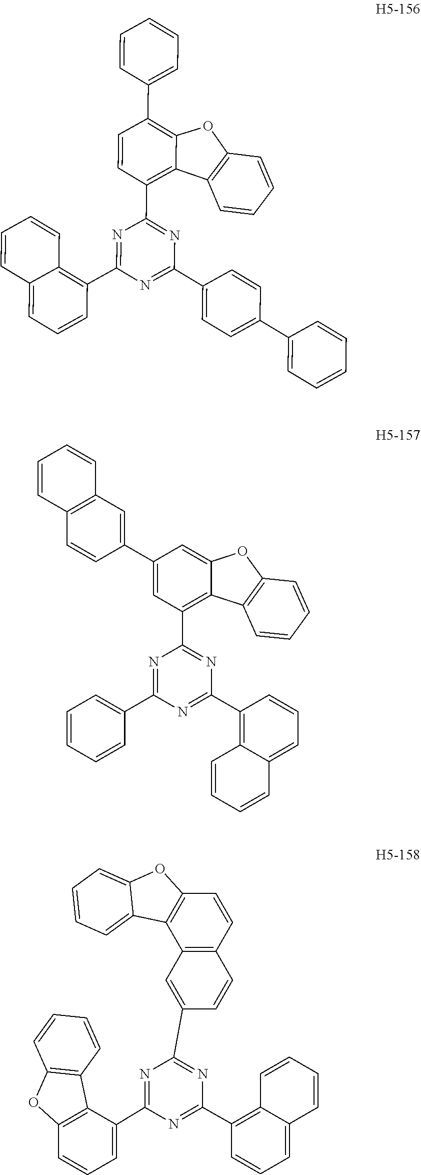 [SMILES: c1ccc(-c2ccc(-c3nc(-c4cccc5ccccc45)nc(-c4ccc(-c5ccccc5)c5oc6ccccc6c45)n3)cc2)cc1 ; c1ccc(-c2nc(-c3cccc4ccccc34)nc(-c3cc(-c4ccc5ccccc5c4)cc4oc5ccccc5c34)n2)cc1 ; c1ccc2c(-c3nc(-c4ccc5ccc6oc7ccccc7c6c5c4)nc(-c4cccc5oc6ccccc6c45)n3)cccc2c1]

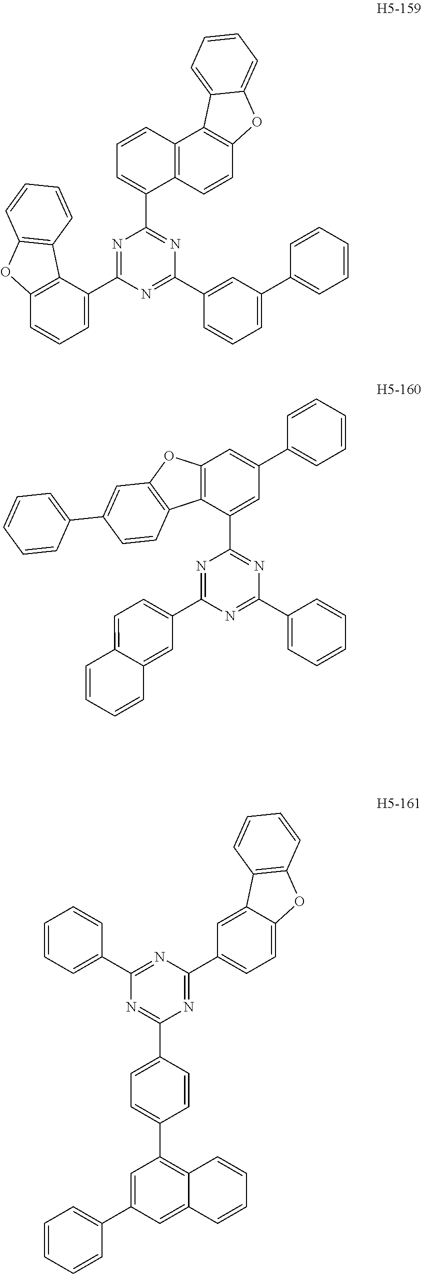 [SMILES: c1ccc(-c2cccc(-c3nc(-c4cccc5c4ccc4oc6ccccc6c45)nc(-c4cccc5oc6ccccc6c45)n3)c2)cc1 ; c1ccc(-c2ccc3c(c2)oc2cc(-c4ccccc4)cc(-c4nc(-c5ccccc5)nc(-c5ccc6ccccc6c5)n4)c23)cc1 ; c1ccc(-c2cc(-c3ccc(-c4nc(-c5ccccc5)nc(-c5ccc6oc7ccccc7c6c5)n4)cc3)c3ccccc3c2)cc1]

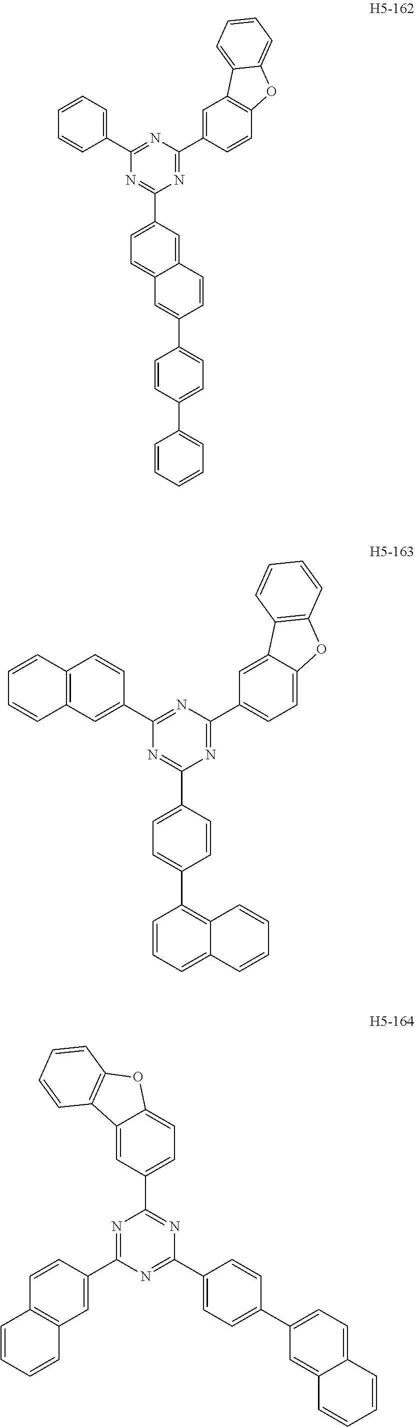 [SMILES: c1ccc(-c2ccc(-c3ccc4cc(-c5nc(-c6ccccc6)nc(-c6ccc7oc8ccccc8c7c6)n5)ccc4c3)cc2)cc1 ; c1ccc2cc(-c3nc(-c4ccc(-c5cccc6ccccc56)cc4)nc(-c4ccc5oc6ccccc6c5c4)n3)ccc2c1 ; c1ccc2cc(-c3ccc(-c4nc(-c5ccc6ccccc6c5)nc(-c5ccc6oc7ccccc7c6c5)n4)cc3)ccc2c1]

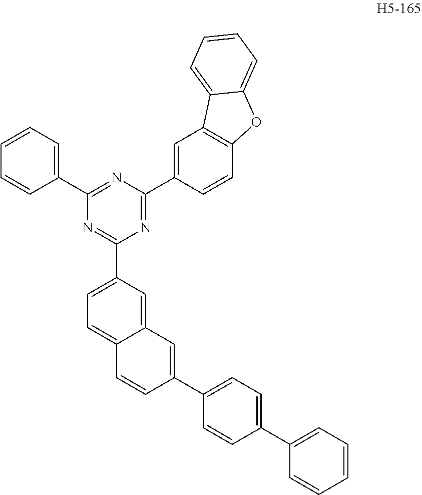 [SMILES: c1ccc(-c2ccc(-c3ccc4ccc(-c5nc(-c6ccccc6)nc(-c6ccc7oc8ccccc8c7c6)n5)cc4c3)cc2)cc1]

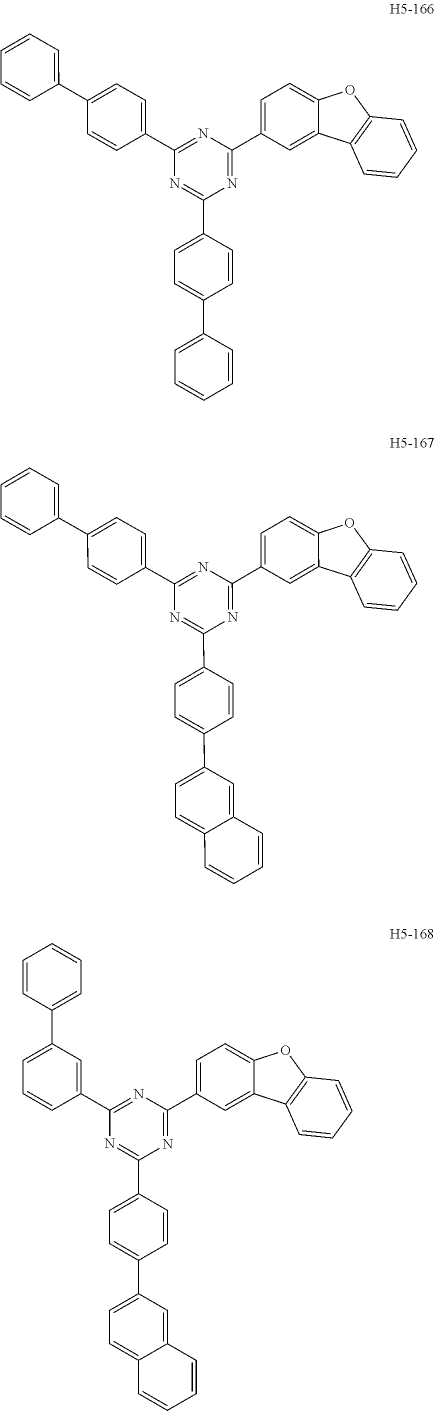 [SMILES: c1ccc(-c2ccc(-c3nc(-c4ccc(-c5ccccc5)cc4)nc(-c4ccc5oc6ccccc6c5c4)n3)cc2)cc1 ; c1ccc(-c2ccc(-c3nc(-c4ccc(-c5ccc6ccccc6c5)cc4)nc(-c4ccc5oc6ccccc6c5c4)n3)cc2)cc1 ; c1ccc(-c2cccc(-c3nc(-c4ccc(-c5ccc6ccccc6c5)cc4)nc(-c4ccc5oc6ccccc6c5c4)n3)c2)cc1]

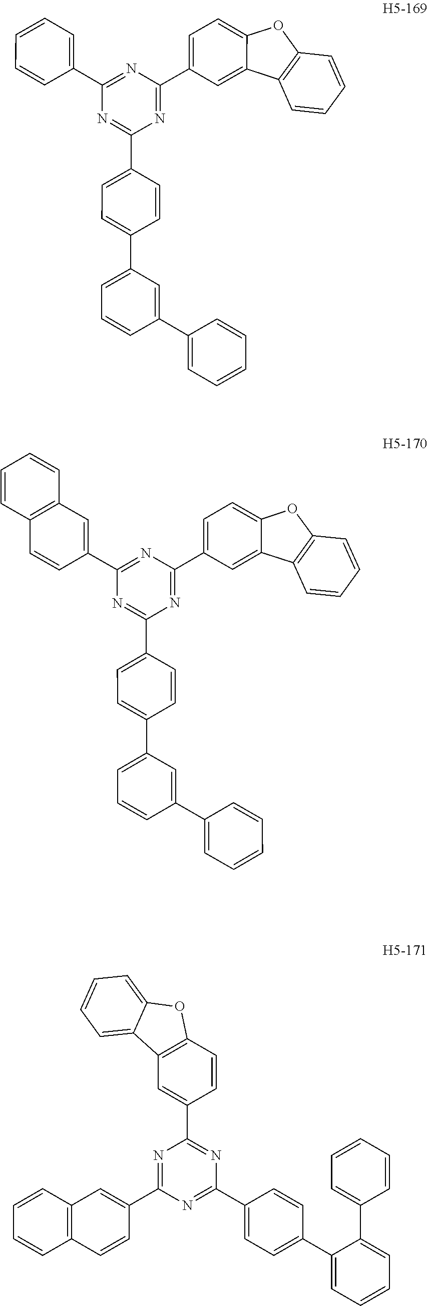 [SMILES: c1ccc(-c2cccc(-c3ccc(-c4nc(-c5ccccc5)nc(-c5ccc6oc7ccccc7c6c5)n4)cc3)c2)cc1 ; c1ccc(-c2cccc(-c3ccc(-c4nc(-c5ccc6ccccc6c5)nc(-c5ccc6oc7ccccc7c6c5)n4)cc3)c2)cc1 ; c1ccc(-c2ccccc2-c2ccc(-c3nc(-c4ccc5ccccc5c4)nc(-c4ccc5oc6ccccc6c5c4)n3)cc2)cc1]

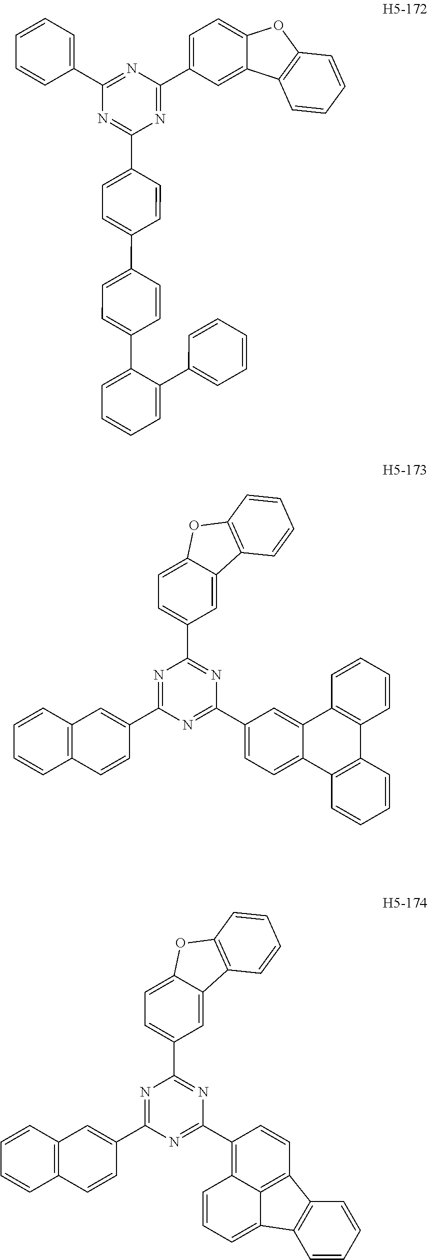 [SMILES: c1ccc(-c2nc(-c3ccc(-c4ccc(-c5ccccc5-c5ccccc5)cc4)cc3)nc(-c3ccc4oc5ccccc5c4c3)n2)cc1 ; c1ccc2cc(-c3nc(-c4ccc5oc6ccccc6c5c4)nc(-c4ccc5c6ccccc6c6ccccc6c5c4)n3)ccc2c1 ; c1ccc2c(c1)-c1cccc3c(-c4nc(-c5ccc6ccccc6c5)nc(-c5ccc6oc7ccccc7c6c5)n4)ccc-2c13]

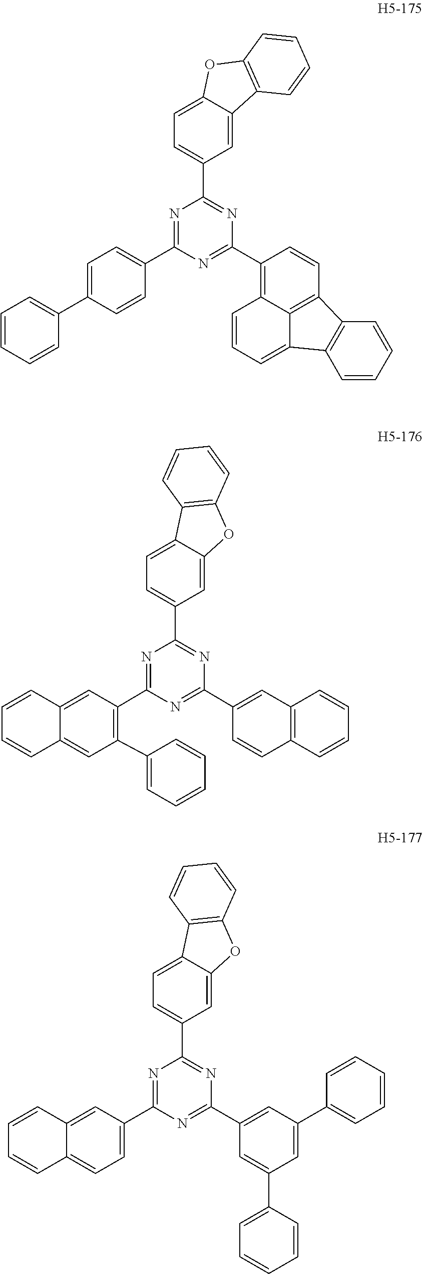 [SMILES: c1ccc(-c2ccc(-c3nc(-c4ccc5oc6ccccc6c5c4)nc(-c4ccc5c6c(cccc46)-c4ccccc4-5)n3)cc2)cc1 ; c1ccc(-c2cc3ccccc3cc2-c2nc(-c3ccc4ccccc4c3)nc(-c3ccc4c(c3)oc3ccccc34)n2)cc1 ; c1ccc(-c2cc(-c3ccccc3)cc(-c3nc(-c4ccc5ccccc5c4)nc(-c4ccc5c(c4)oc4ccccc45)n3)c2)cc1]

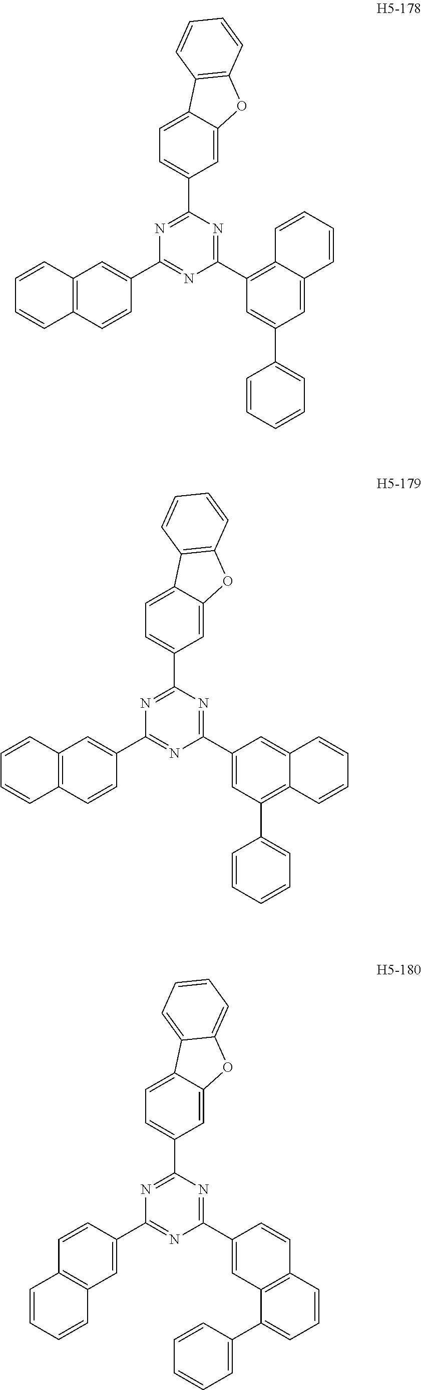 [SMILES: c1ccc(-c2cc(-c3nc(-c4ccc5ccccc5c4)nc(-c4ccc5c(c4)oc4ccccc45)n3)c3ccccc3c2)cc1 ; c1ccc(-c2cc(-c3nc(-c4ccc5ccccc5c4)nc(-c4ccc5c(c4)oc4ccccc45)n3)cc3ccccc23)cc1 ; c1ccc(-c2cccc3ccc(-c4nc(-c5ccc6ccccc6c5)nc(-c5ccc6c(c5)oc5ccccc56)n4)cc23)cc1]

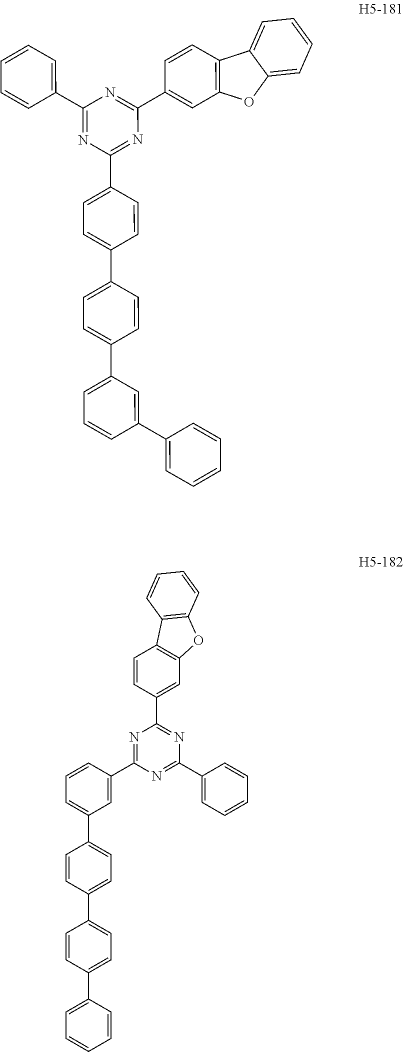 [SMILES: c1ccc(-c2cccc(-c3ccc(-c4ccc(-c5nc(-c6ccccc6)nc(-c6ccc7c(c6)oc6ccccc67)n5)cc4)cc3)c2)cc1 ; c1ccc(-c2ccc(-c3ccc(-c4cccc(-c5nc(-c6ccccc6)nc(-c6ccc7c(c6)oc6ccccc67)n5)c4)cc3)cc2)cc1]

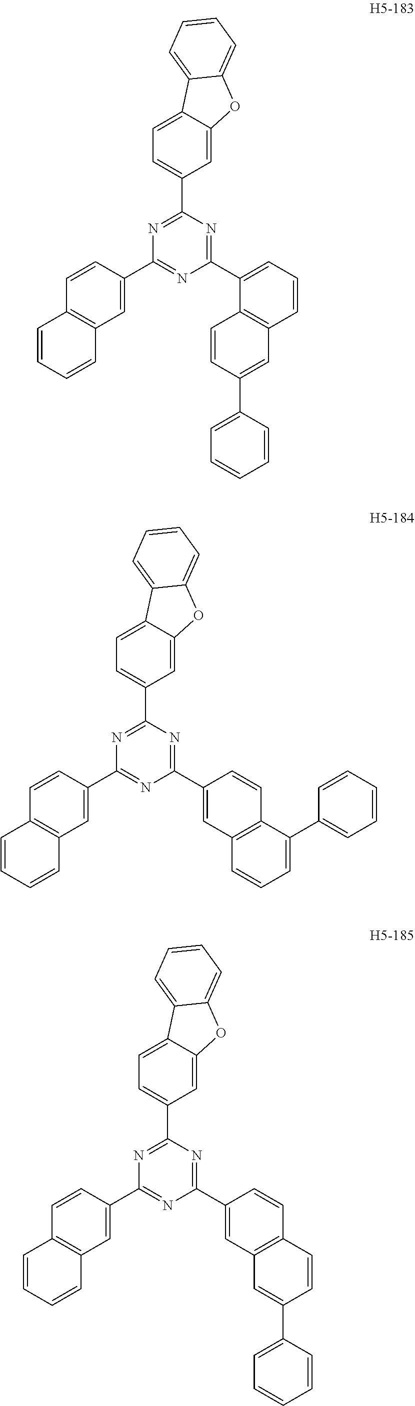 [SMILES: c1ccc(-c2ccc3c(-c4nc(-c5ccc6ccccc6c5)nc(-c5ccc6c(c5)oc5ccccc56)n4)cccc3c2)cc1 ; c1ccc(-c2cccc3cc(-c4nc(-c5ccc6ccccc6c5)nc(-c5ccc6c(c5)oc5ccccc56)n4)ccc23)cc1 ; c1ccc(-c2ccc3ccc(-c4nc(-c5ccc6ccccc6c5)nc(-c5ccc6c(c5)oc5ccccc56)n4)cc3c2)cc1]

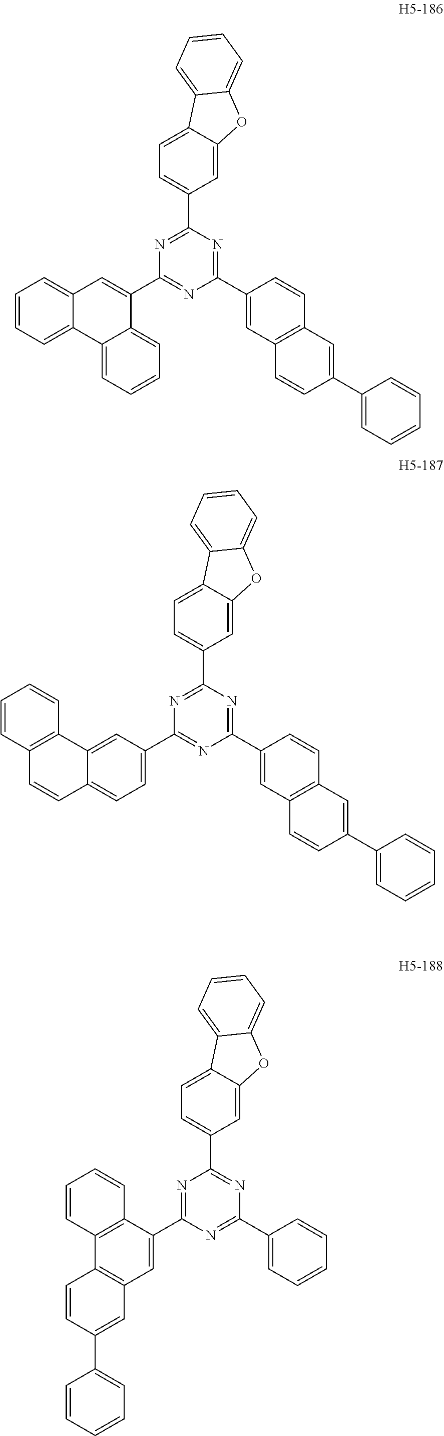 [SMILES: c1ccc(-c2ccc3cc(-c4nc(-c5ccc6c(c5)oc5ccccc56)nc(-c5cc6ccccc6c6ccccc56)n4)ccc3c2)cc1 ; c1ccc(-c2ccc3cc(-c4nc(-c5ccc6c(c5)oc5ccccc56)nc(-c5ccc6ccc7ccccc7c6c5)n4)ccc3c2)cc1 ; c1ccc(-c2ccc3c(c2)cc(-c2nc(-c4ccccc4)nc(-c4ccc5c(c4)oc4ccccc45)n2)c2ccccc23)cc1]

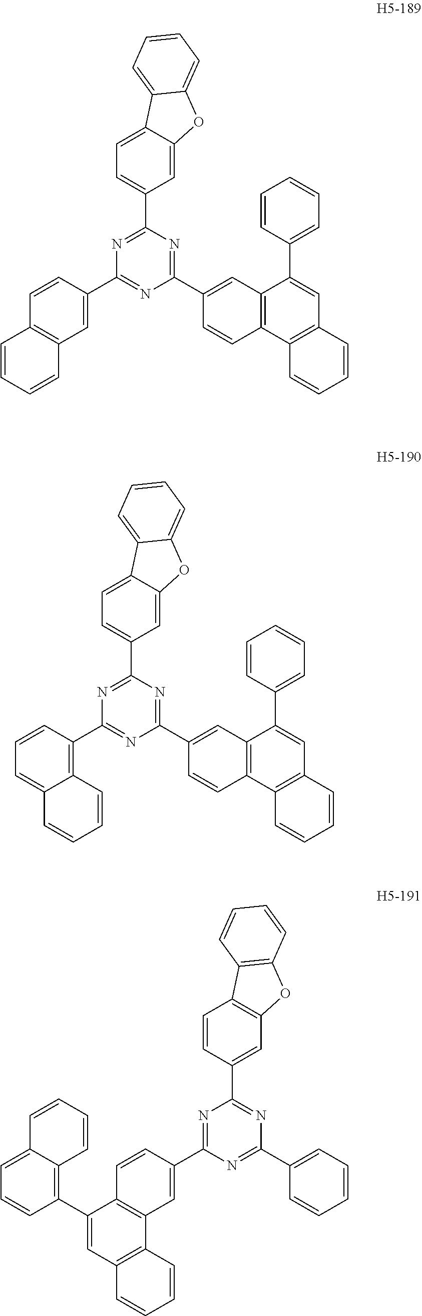 [SMILES: c1ccc(-c2cc3ccccc3c3ccc(-c4nc(-c5ccc6ccccc6c5)nc(-c5ccc6c(c5)oc5ccccc56)n4)cc23)cc1 ; c1ccc(-c2cc3ccccc3c3ccc(-c4nc(-c5ccc6c(c5)oc5ccccc56)nc(-c5cccc6ccccc56)n4)cc23)cc1 ; c1ccc(-c2nc(-c3ccc4c(c3)oc3ccccc34)nc(-c3ccc4c(-c5cccc6ccccc56)cc5ccccc5c4c3)n2)cc1]

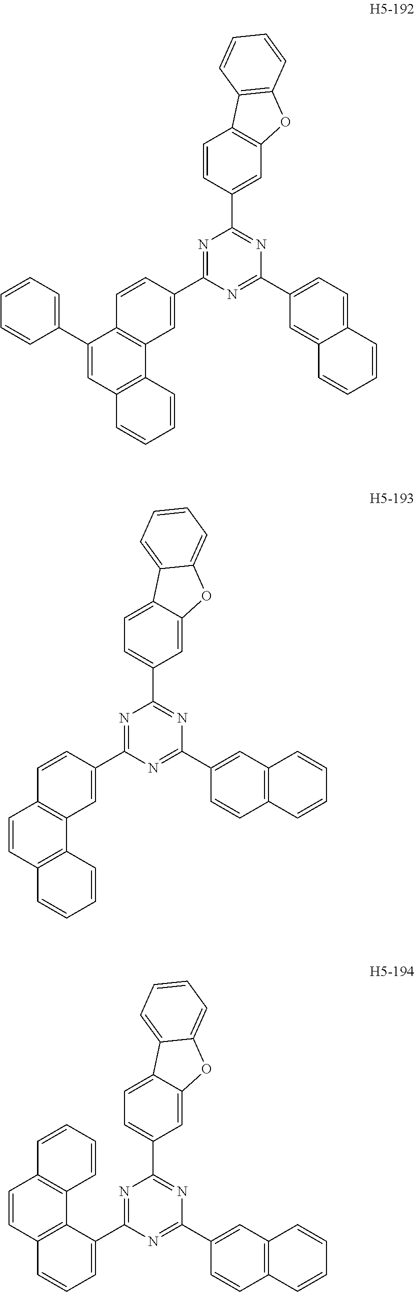 [SMILES: c1ccc(-c2cc3ccccc3c3cc(-c4nc(-c5ccc6ccccc6c5)nc(-c5ccc6c(c5)oc5ccccc56)n4)ccc23)cc1 ; c1ccc2cc(-c3nc(-c4ccc5c(c4)oc4ccccc45)nc(-c4ccc5ccc6ccccc6c5c4)n3)ccc2c1 ; c1ccc2cc(-c3nc(-c4ccc5c(c4)oc4ccccc45)nc(-c4cccc5ccc6ccccc6c45)n3)ccc2c1]

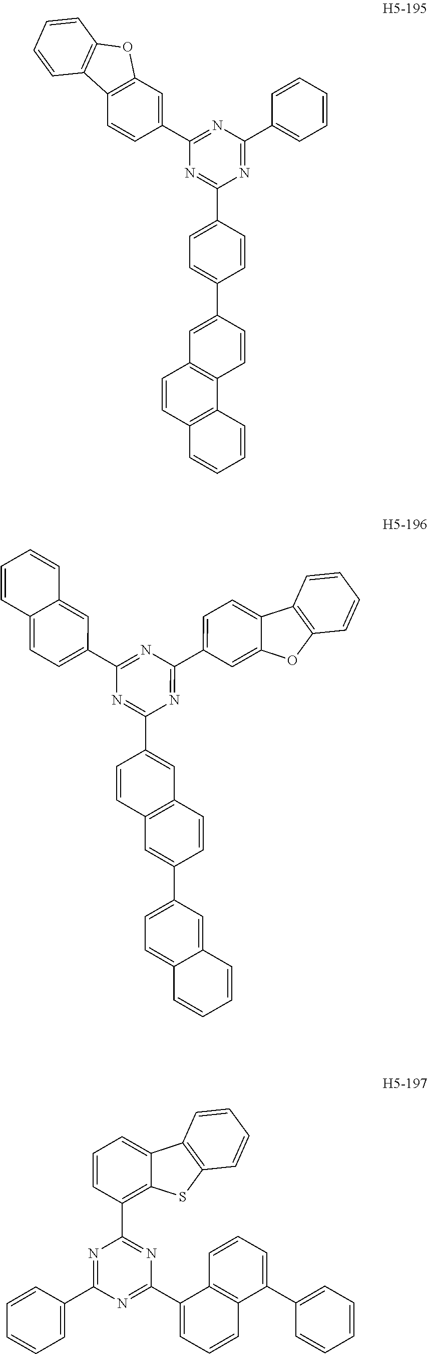 [SMILES: c1ccc(-c2nc(-c3ccc(-c4ccc5c(ccc6ccccc65)c4)cc3)nc(-c3ccc4c(c3)oc3ccccc34)n2)cc1 ; c1ccc2cc(-c3ccc4cc(-c5nc(-c6ccc7ccccc7c6)nc(-c6ccc7c(c6)oc6ccccc67)n5)ccc4c3)ccc2c1 ; c1ccc(-c2nc(-c3cccc4c(-c5ccccc5)cccc34)nc(-c3cccc4c3sc3ccccc34)n2)cc1]

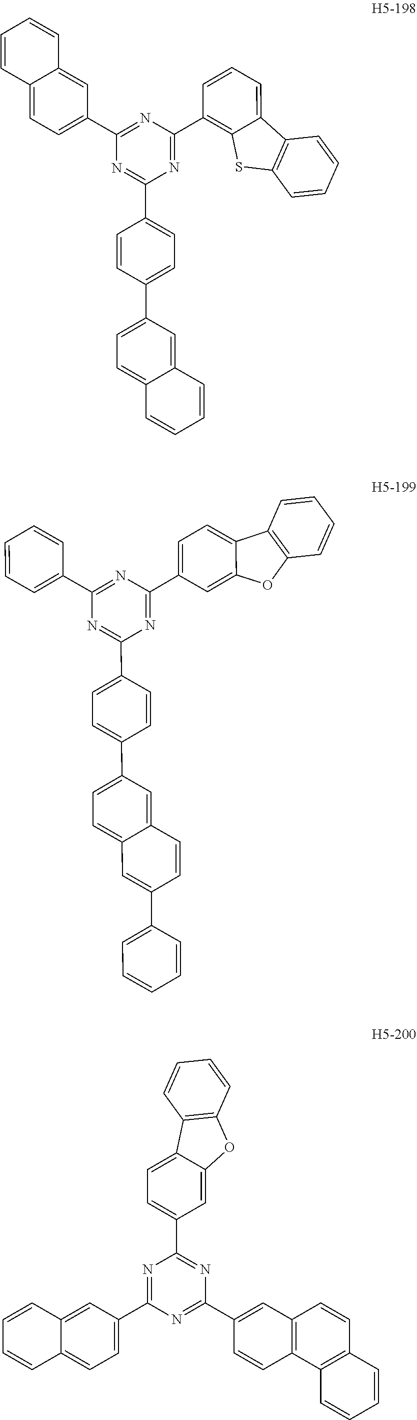 [SMILES: c1ccc2cc(-c3ccc(-c4nc(-c5ccc6ccccc6c5)nc(-c5cccc6c5sc5ccccc56)n4)cc3)ccc2c1 ; c1ccc(-c2ccc3cc(-c4ccc(-c5nc(-c6ccccc6)nc(-c6ccc7c(c6)oc6ccccc67)n5)cc4)ccc3c2)cc1 ; c1ccc2cc(-c3nc(-c4ccc5c(ccc6ccccc65)c4)nc(-c4ccc5c(c4)oc4ccccc45)n3)ccc2c1]

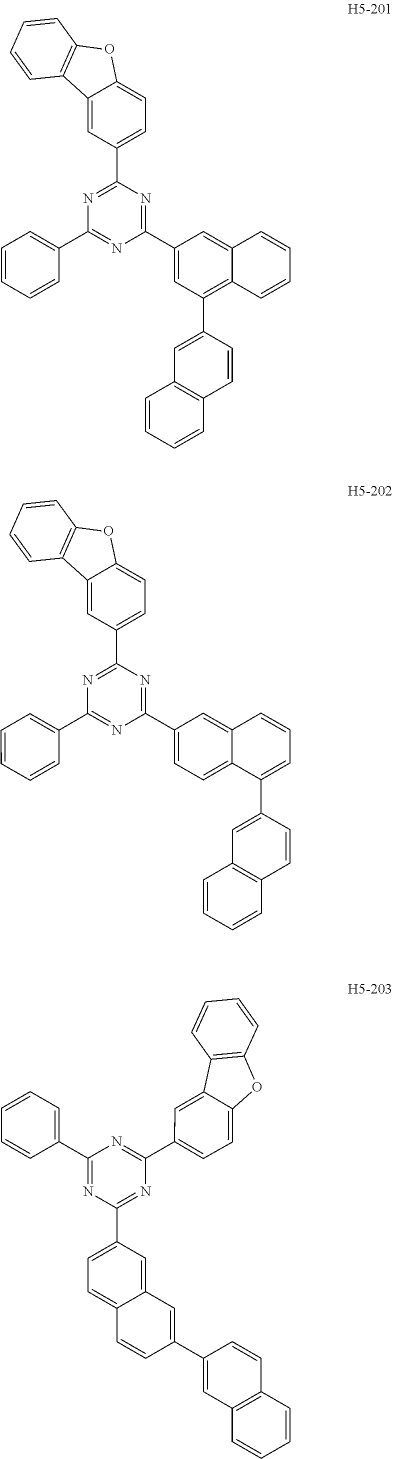 [SMILES: c1ccc(-c2nc(-c3cc(-c4ccc5ccccc5c4)c4ccccc4c3)nc(-c3ccc4oc5ccccc5c4c3)n2)cc1 ; c1ccc(-c2nc(-c3ccc4c(-c5ccc6ccccc6c5)cccc4c3)nc(-c3ccc4oc5ccccc5c4c3)n2)cc1 ; c1ccc(-c2nc(-c3ccc4ccc(-c5ccc6ccccc6c5)cc4c3)nc(-c3ccc4oc5ccccc5c4c3)n2)cc1]

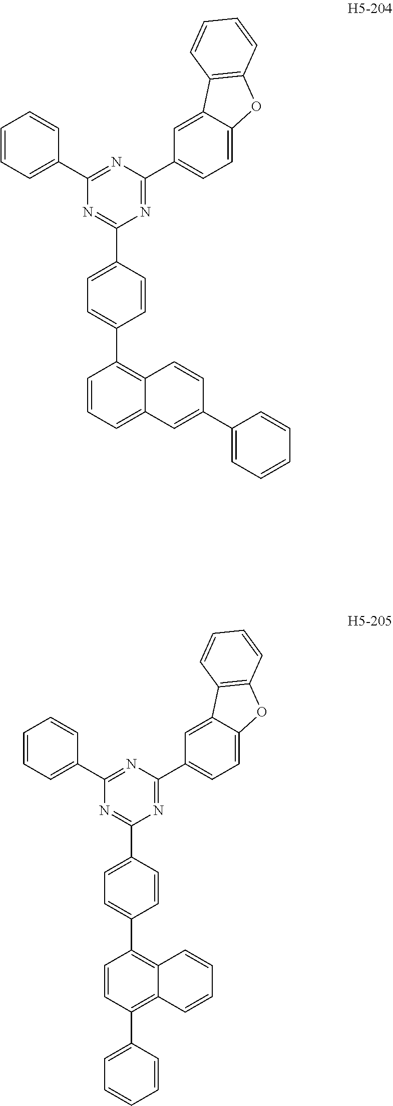 [SMILES: c1ccc(-c2ccc3c(-c4ccc(-c5nc(-c6ccccc6)nc(-c6ccc7oc8ccccc8c7c6)n5)cc4)cccc3c2)cc1 ; c1ccc(-c2nc(-c3ccc(-c4ccc(-c5ccccc5)c5ccccc45)cc3)nc(-c3ccc4oc5ccccc5c4c3)n2)cc1]

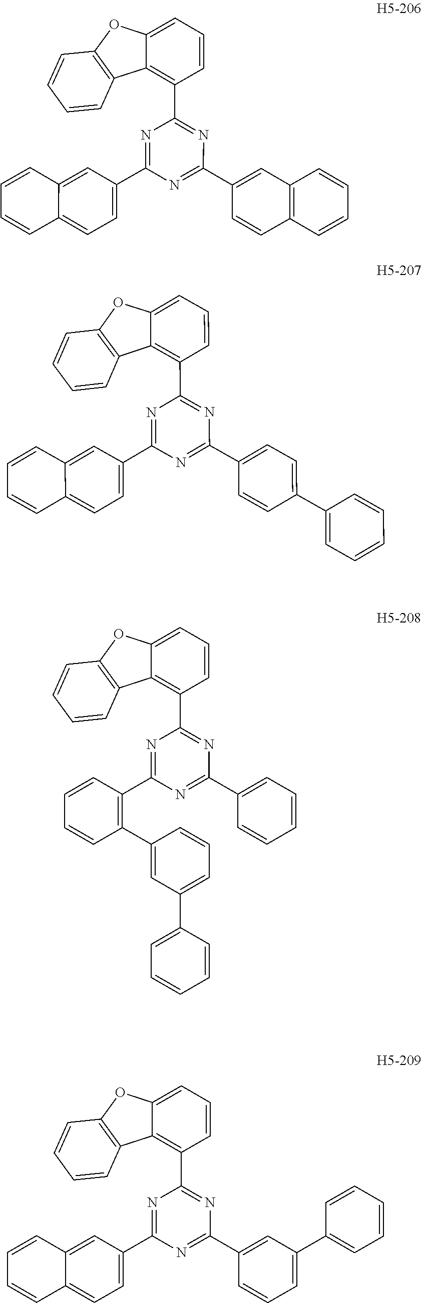 [SMILES: c1ccc2cc(-c3nc(-c4ccc5ccccc5c4)nc(-c4cccc5oc6ccccc6c45)n3)ccc2c1 ; c1ccc(-c2ccc(-c3nc(-c4ccc5ccccc5c4)nc(-c4cccc5oc6ccccc6c45)n3)cc2)cc1 ; c1ccc(-c2cccc(-c3ccccc3-c3nc(-c4ccccc4)nc(-c4cccc5oc6ccccc6c45)n3)c2)cc1 ; c1ccc(-c2cccc(-c3nc(-c4ccc5ccccc5c4)nc(-c4cccc5oc6ccccc6c45)n3)c2)cc1]

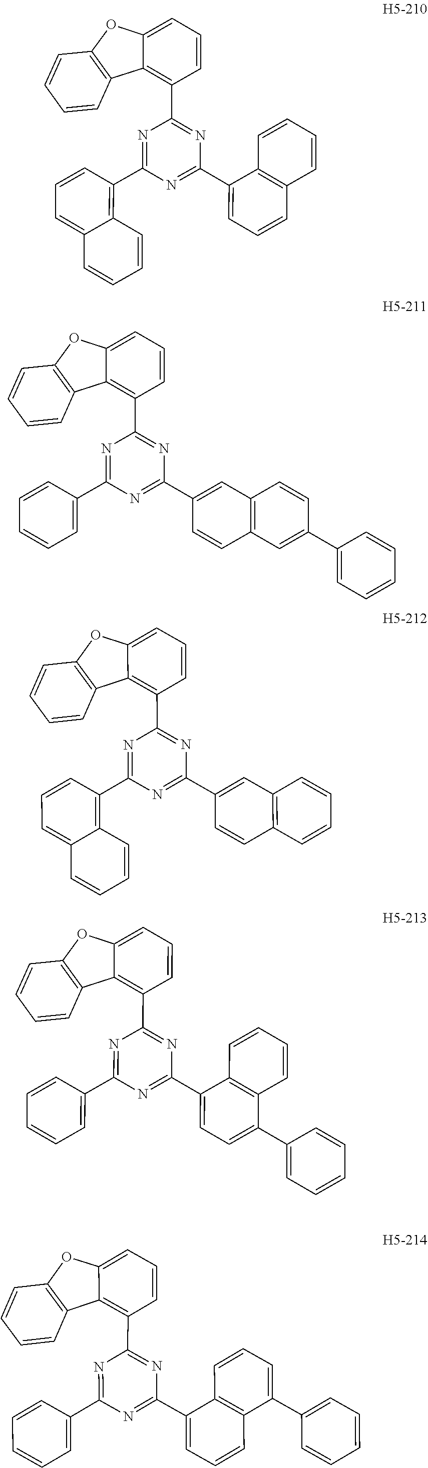

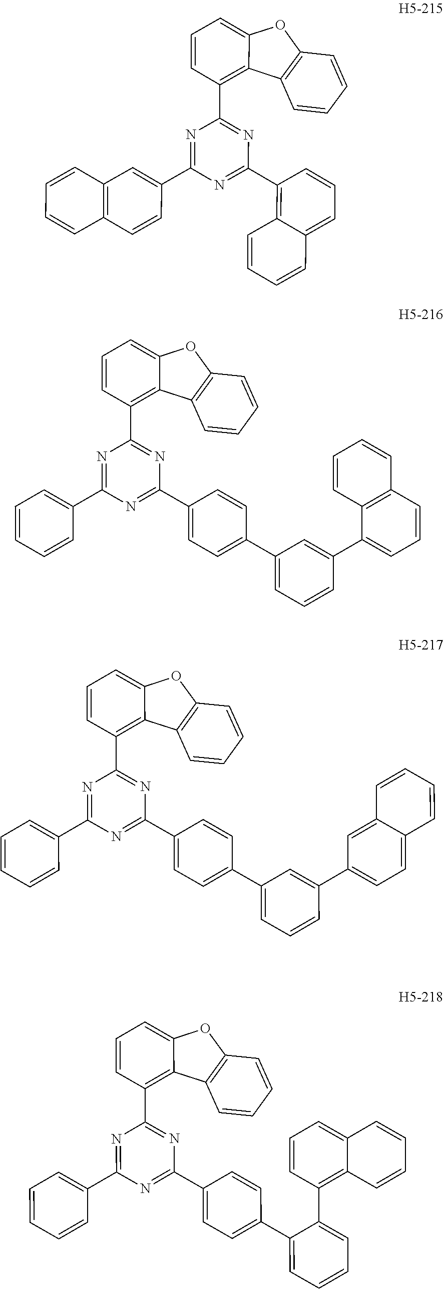 [SMILES: c1ccc2cc(-c3nc(-c4cccc5ccccc45)nc(-c4cccc5oc6ccccc6c45)n3)ccc2c1 ; c1ccc(-c2nc(-c3ccc(-c4cccc(-c5cccc6ccccc56)c4)cc3)nc(-c3cccc4oc5ccccc5c34)n2)cc1 ; c1ccc(-c2nc(-c3ccc(-c4cccc(-c5ccc6ccccc6c5)c4)cc3)nc(-c3cccc4oc5ccccc5c34)n2)cc1 ; c1ccc(-c2nc(-c3ccc(-c4ccccc4-c4cccc5ccccc45)cc3)nc(-c3cccc4oc5ccccc5c34)n2)cc1]

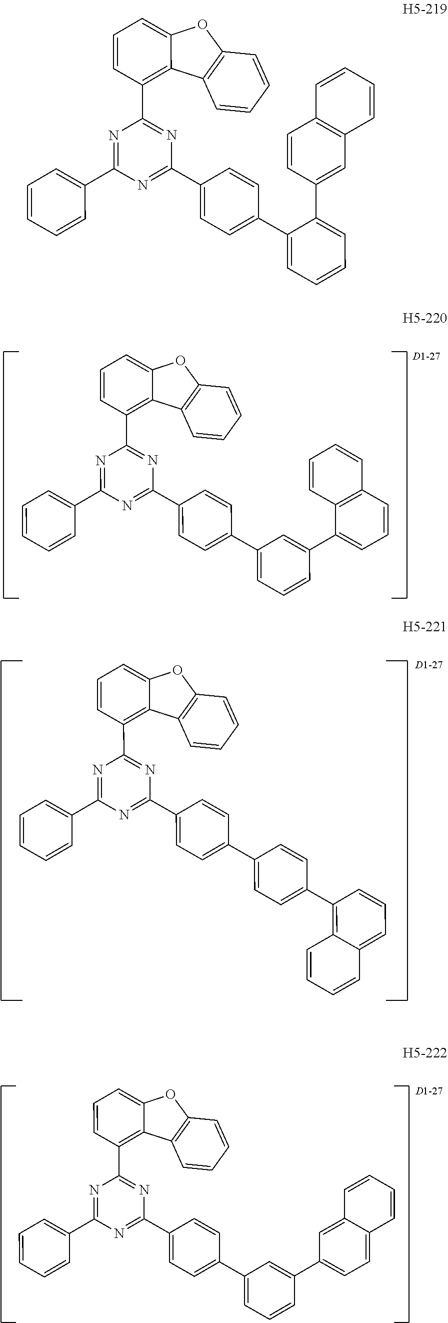

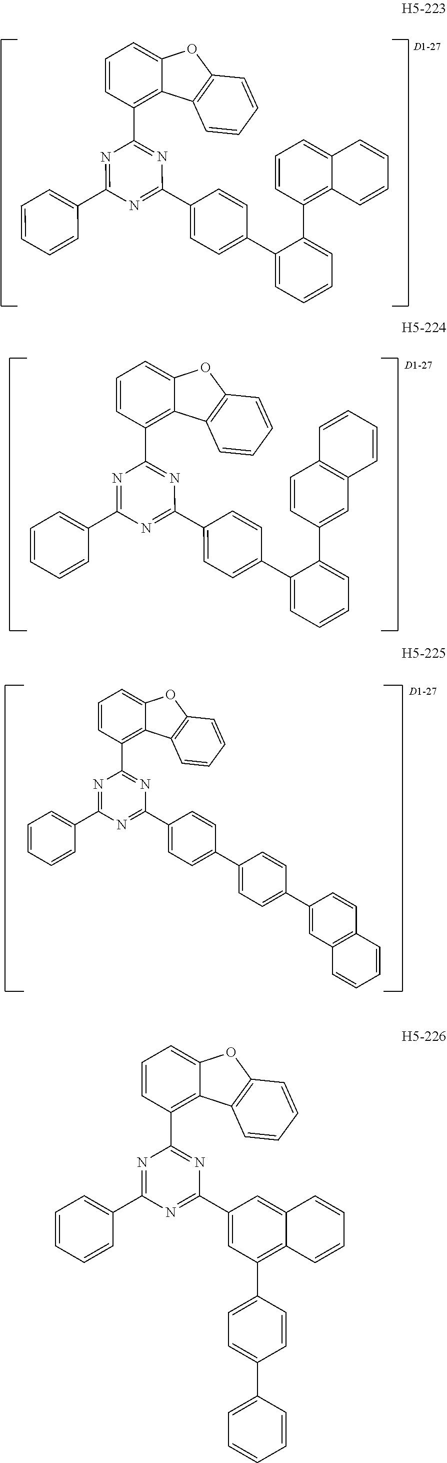

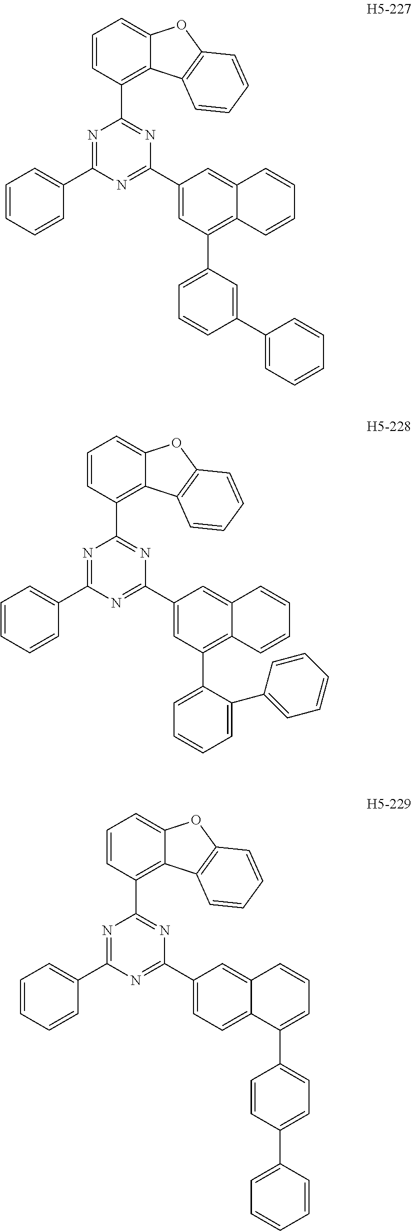 [SMILES: c1ccc(-c2cccc(-c3cc(-c4nc(-c5ccccc5)nc(-c5cccc6oc7ccccc7c56)n4)cc4ccccc34)c2)cc1 ; c1ccc(-c2nc(-c3cc(-c4ccccc4-c4ccccc4)c4ccccc4c3)nc(-c3cccc4oc5ccccc5c34)n2)cc1 ; c1ccc(-c2ccc(-c3cccc4cc(-c5nc(-c6ccccc6)nc(-c6cccc7oc8ccccc8c67)n5)ccc34)cc2)cc1]

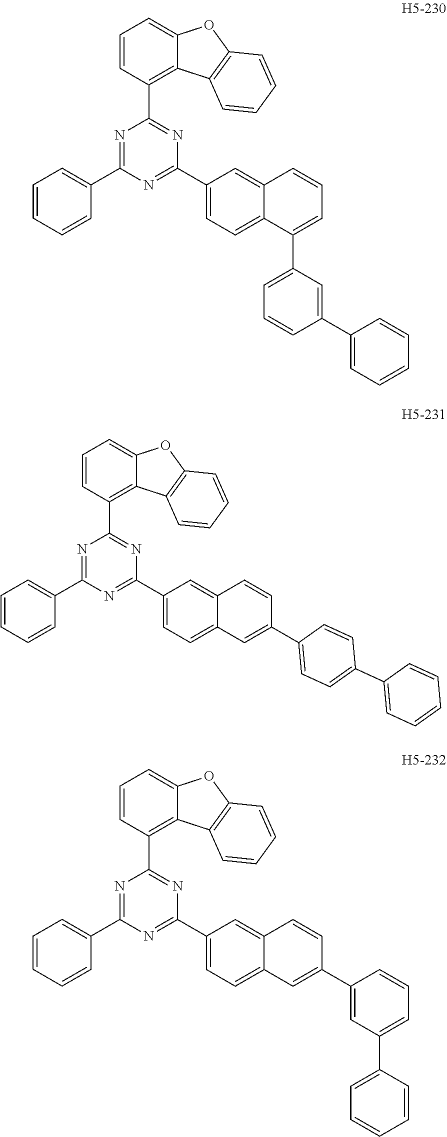 [SMILES: c1ccc(-c2cccc(-c3cccc4cc(-c5nc(-c6ccccc6)nc(-c6cccc7oc8ccccc8c67)n5)ccc34)c2)cc1 ; c1ccc(-c2ccc(-c3ccc4cc(-c5nc(-c6ccccc6)nc(-c6cccc7oc8ccccc8c67)n5)ccc4c3)cc2)cc1 ; c1ccc(-c2cccc(-c3ccc4cc(-c5nc(-c6ccccc6)nc(-c6cccc7oc8ccccc8c67)n5)ccc4c3)c2)cc1]

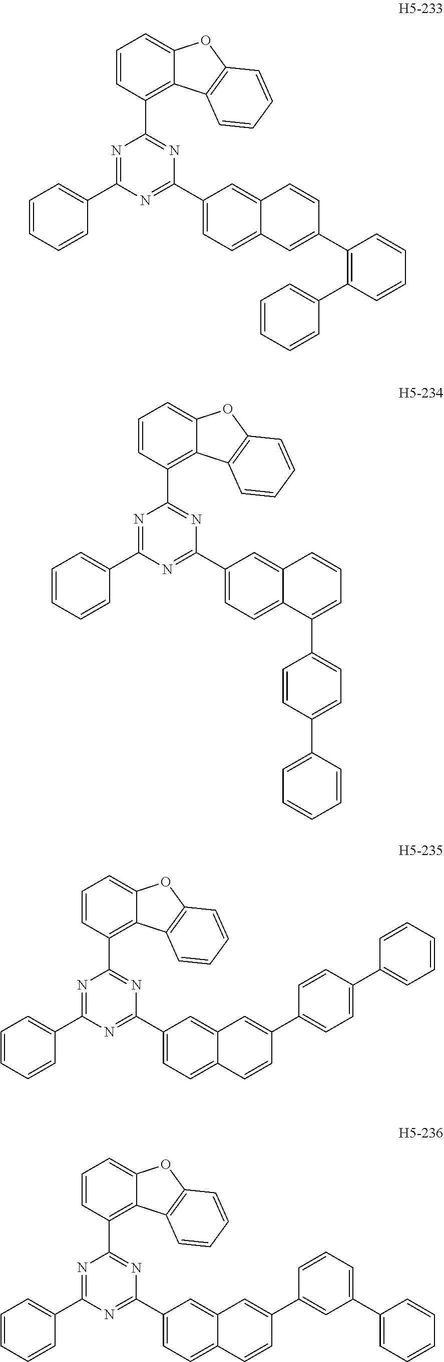 [SMILES: c1ccc(-c2nc(-c3ccc4cc(-c5ccccc5-c5ccccc5)ccc4c3)nc(-c3cccc4oc5ccccc5c34)n2)cc1 ; c1ccc(-c2ccc(-c3cccc4cc(-c5nc(-c6ccccc6)nc(-c6cccc7oc8ccccc8c67)n5)ccc34)cc2)cc1 ; c1ccc(-c2ccc(-c3ccc4ccc(-c5nc(-c6ccccc6)nc(-c6cccc7oc8ccccc8c67)n5)cc4c3)cc2)cc1 ; c1ccc(-c2cccc(-c3ccc4ccc(-c5nc(-c6ccccc6)nc(-c6cccc7oc8ccccc8c67)n5)cc4c3)c2)cc1]

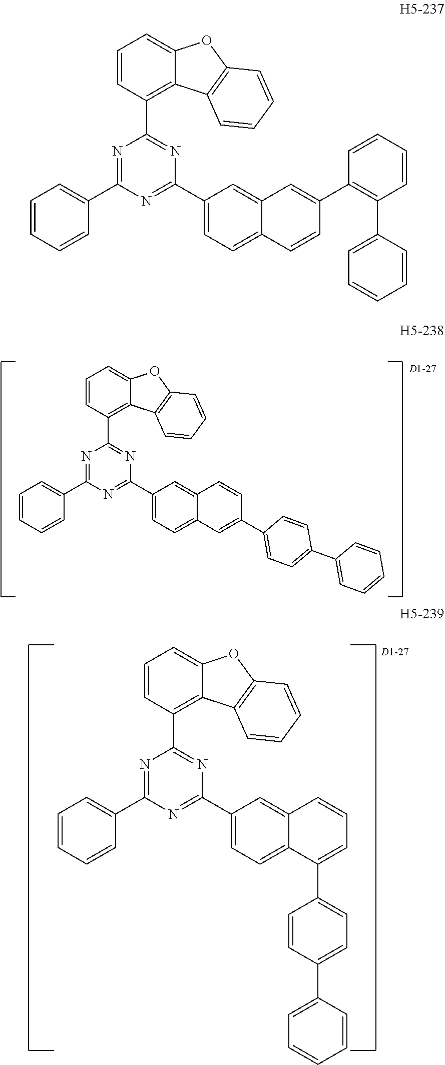

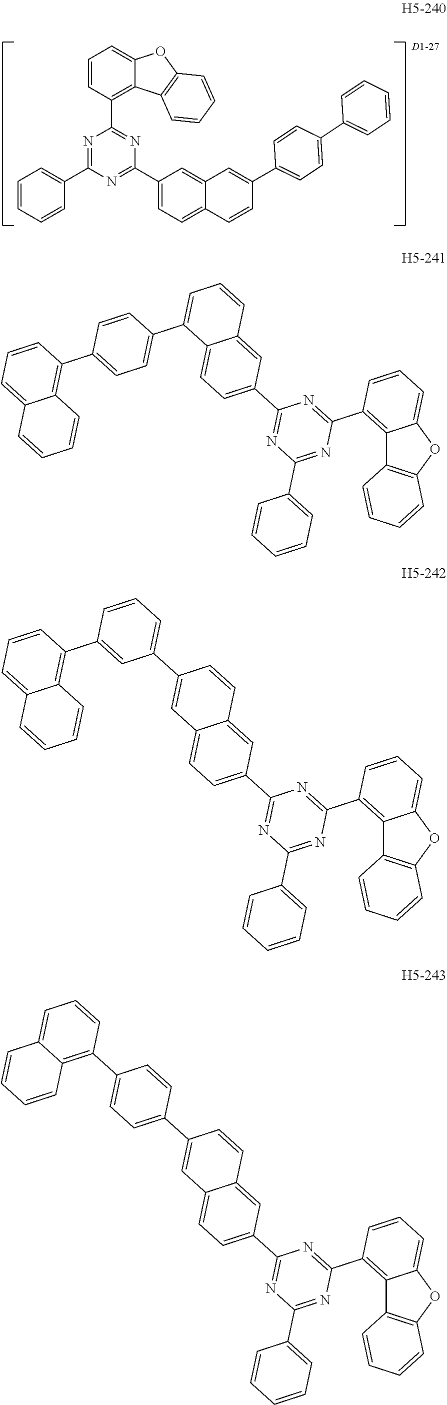

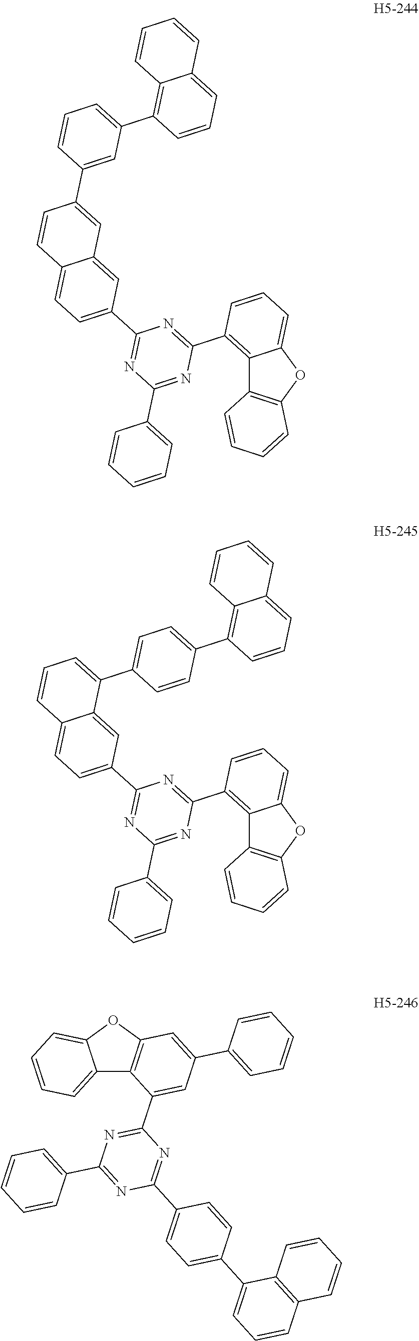 [SMILES: c1ccc(-c2nc(-c3ccc4ccc(-c5cccc(-c6cccc7ccccc67)c5)cc4c3)nc(-c3cccc4oc5ccccc5c34)n2)cc1 ; c1ccc(-c2nc(-c3ccc4cccc(-c5ccc(-c6cccc7ccccc67)cc5)c4c3)nc(-c3cccc4oc5ccccc5c34)n2)cc1 ; c1ccc(-c2cc(-c3nc(-c4ccccc4)nc(-c4ccc(-c5cccc6ccccc56)cc4)n3)c3c(c2)oc2ccccc23)cc1]

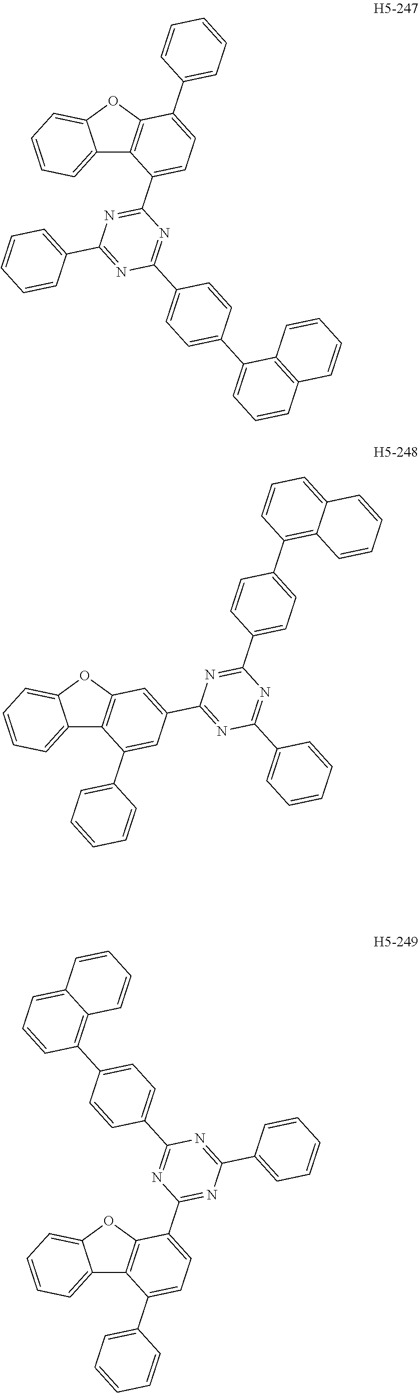 [SMILES: c1ccc(-c2nc(-c3ccc(-c4cccc5ccccc45)cc3)nc(-c3ccc(-c4ccccc4)c4oc5ccccc5c34)n2)cc1 ; c1ccc(-c2nc(-c3ccc(-c4cccc5ccccc45)cc3)nc(-c3cc(-c4ccccc4)c4c(c3)oc3ccccc34)n2)cc1 ; c1ccc(-c2nc(-c3ccc(-c4cccc5ccccc45)cc3)nc(-c3ccc(-c4ccccc4)c4c3oc3ccccc34)n2)cc1]

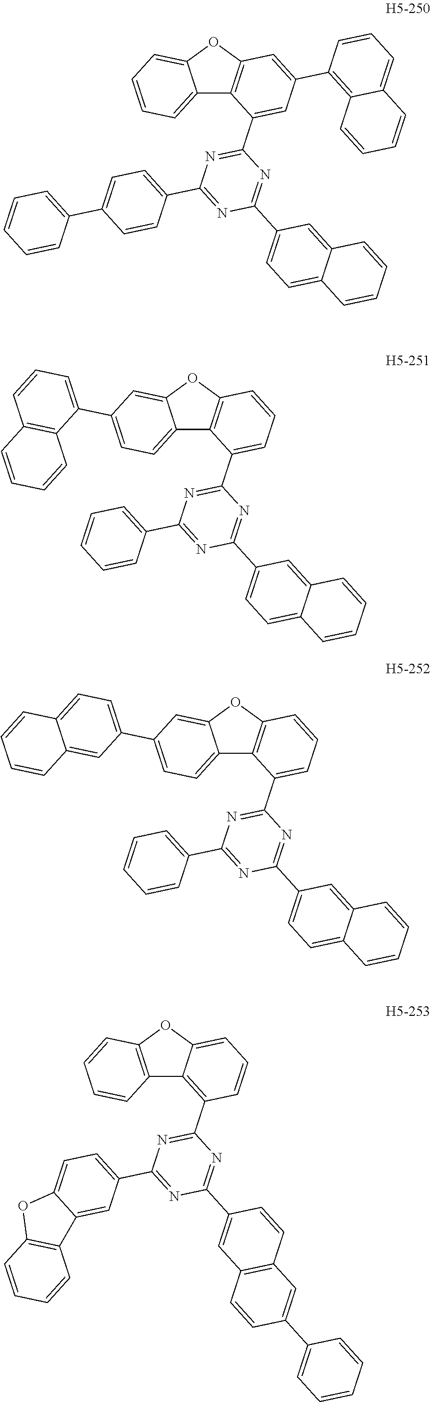 [SMILES: c1ccc(-c2ccc(-c3nc(-c4ccc5ccccc5c4)nc(-c4cc(-c5cccc6ccccc56)cc5oc6ccccc6c45)n3)cc2)cc1 ; c1ccc(-c2nc(-c3ccc4ccccc4c3)nc(-c3cccc4oc5cc(-c6cccc7ccccc67)ccc5c34)n2)cc1 ; c1ccc(-c2nc(-c3ccc4ccccc4c3)nc(-c3cccc4oc5cc(-c6ccc7ccccc7c6)ccc5c34)n2)cc1 ; c1ccc(-c2ccc3cc(-c4nc(-c5ccc6oc7ccccc7c6c5)nc(-c5cccc6oc7ccccc7c56)n4)ccc3c2)cc1]

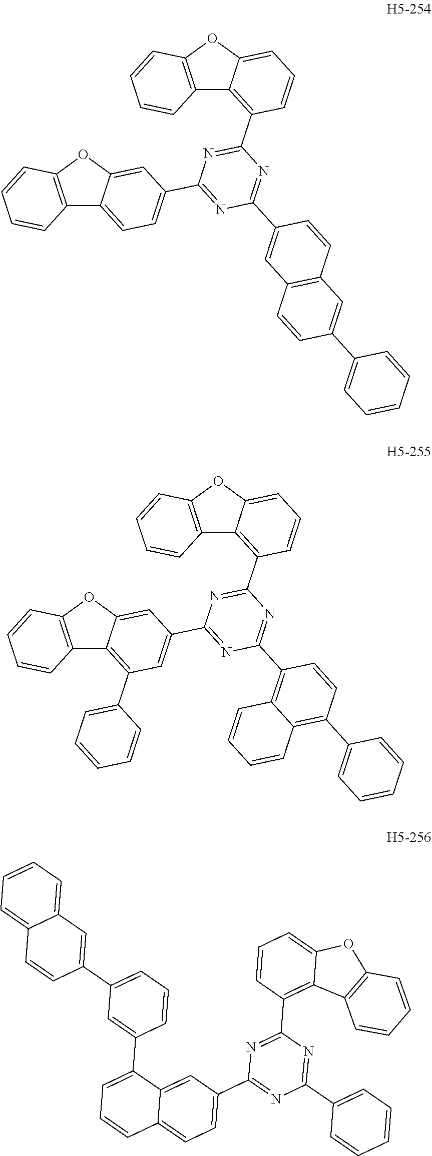 [SMILES: c1ccc(-c2ccc3cc(-c4nc(-c5ccc6c(c5)oc5ccccc56)nc(-c5cccc6oc7ccccc7c56)n4)ccc3c2)cc1 ; c1ccc(-c2ccc(-c3nc(-c4cc(-c5ccccc5)c5c(c4)oc4ccccc45)nc(-c4cccc5oc6ccccc6c45)n3)c3ccccc23)cc1 ; c1ccc(-c2nc(-c3ccc4cccc(-c5cccc(-c6ccc7ccccc7c6)c5)c4c3)nc(-c3cccc4oc5ccccc5c34)n2)cc1]

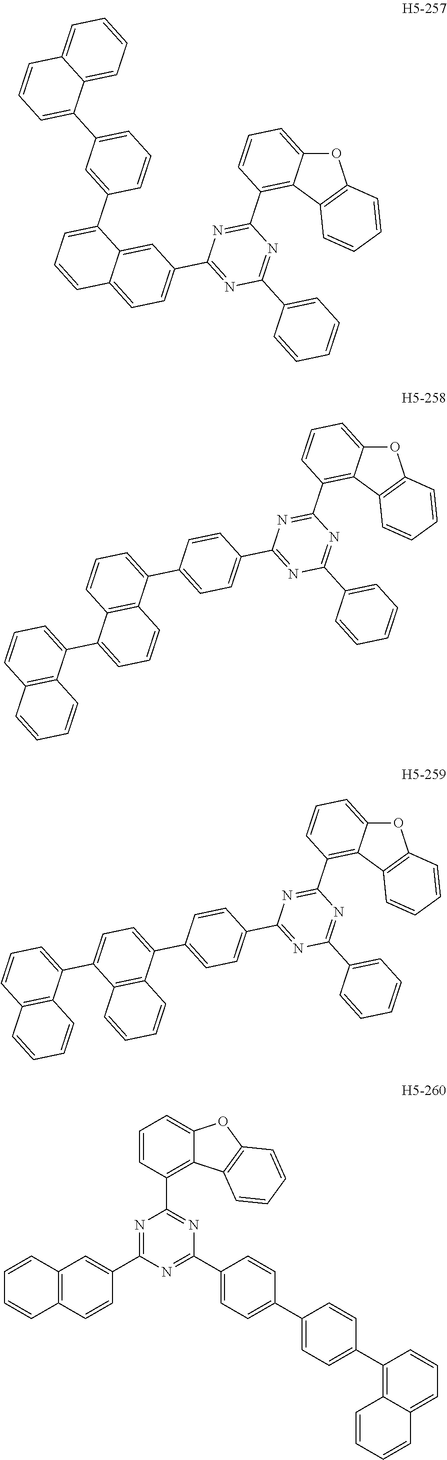 [SMILES: c1ccc(-c2nc(-c3ccc4cccc(-c5cccc(-c6cccc7ccccc67)c5)c4c3)nc(-c3cccc4oc5ccccc5c34)n2)cc1 ; c1ccc(-c2nc(-c3ccc(-c4cccc5c(-c6cccc7ccccc67)cccc45)cc3)nc(-c3cccc4oc5ccccc5c34)n2)cc1 ; c1ccc(-c2nc(-c3ccc(-c4ccc(-c5cccc6ccccc56)c5ccccc45)cc3)nc(-c3cccc4oc5ccccc5c34)n2)cc1 ; c1ccc2cc(-c3nc(-c4ccc(-c5ccc(-c6cccc7ccccc67)cc5)cc4)nc(-c4cccc5oc6ccccc6c45)n3)ccc2c1]

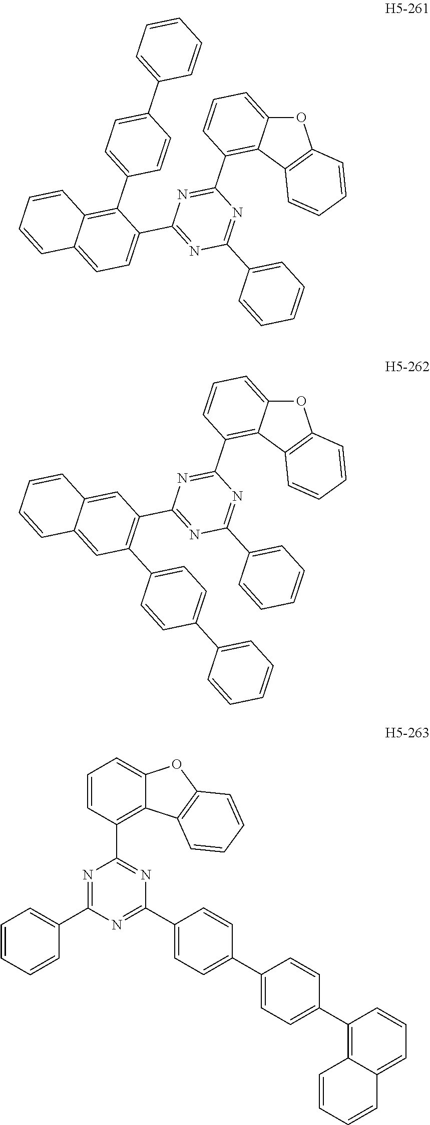 [SMILES: c1ccc(-c2ccc(-c3c(-c4nc(-c5ccccc5)nc(-c5cccc6oc7ccccc7c56)n4)ccc4ccccc34)cc2)cc1 ; c1ccc(-c2ccc(-c3cc4ccccc4cc3-c3nc(-c4ccccc4)nc(-c4cccc5oc6ccccc6c45)n3)cc2)cc1 ; c1ccc(-c2nc(-c3ccc(-c4ccc(-c5cccc6ccccc56)cc4)cc3)nc(-c3cccc4oc5ccccc5c34)n2)cc1]

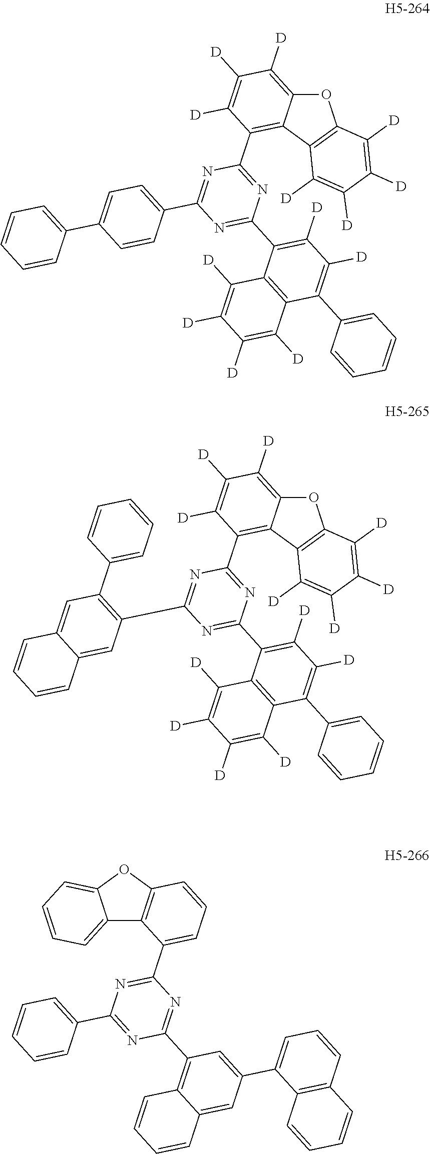 [SMILES: [2H]c1c([2H])c([2H])c2c(oc3c([2H])c([2H])c([2H])c(-c4nc(-c5ccc(-c6ccccc6)cc5)nc(-c5c([2H])c([2H])c(-c6ccccc6)c6c([2H])c([2H])c([2H])c([2H])c56)n4)c32)c1[2H] ; [2H]c1c([2H])c([2H])c2c(oc3c([2H])c([2H])c([2H])c(-c4nc(-c5cc6ccccc6cc5-c5ccccc5)nc(-c5c([2H])c([2H])c(-c6ccccc6)c6c([2H])c([2H])c([2H])c([2H])c56)n4)c32)c1[2H] ; c1ccc(-c2nc(-c3cc(-c4cccc5ccccc45)cc4ccccc34)nc(-c3cccc4oc5ccccc5c34)n2)cc1]

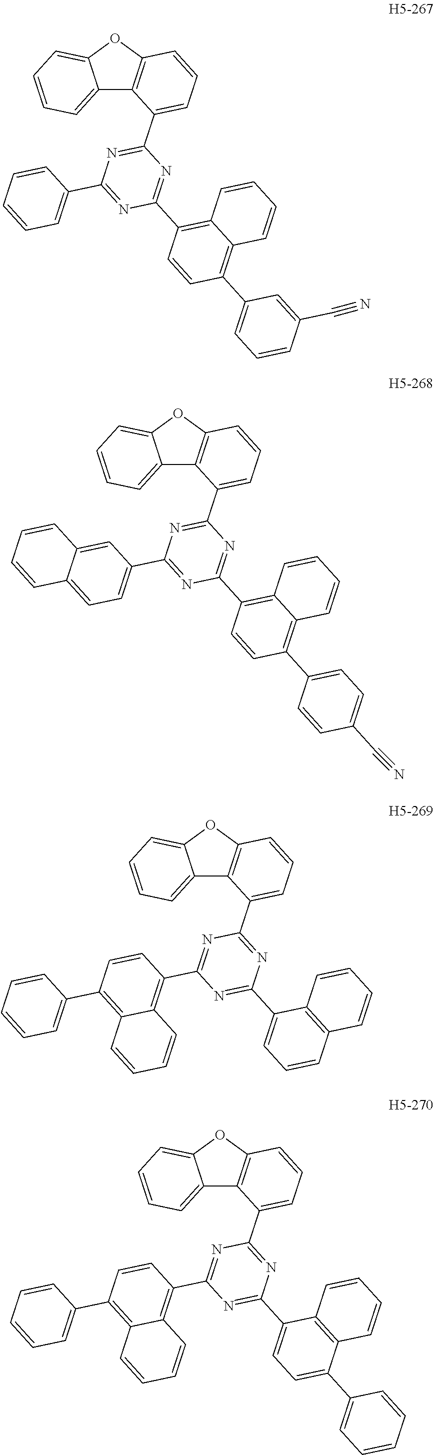 [SMILES: N#Cc1cccc(-c2ccc(-c3nc(-c4ccccc4)nc(-c4cccc5oc6ccccc6c45)n3)c3ccccc23)c1 ; N#Cc1ccc(-c2ccc(-c3nc(-c4ccc5ccccc5c4)nc(-c4cccc5oc6ccccc6c45)n3)c3ccccc23)cc1 ; c1ccc(-c2ccc(-c3nc(-c4cccc5ccccc45)nc(-c4cccc5oc6ccccc6c45)n3)c3ccccc23)cc1 ; c1ccc(-c2ccc(-c3nc(-c4ccc(-c5ccccc5)c5ccccc45)nc(-c4cccc5oc6ccccc6c45)n3)c3ccccc23)cc1]

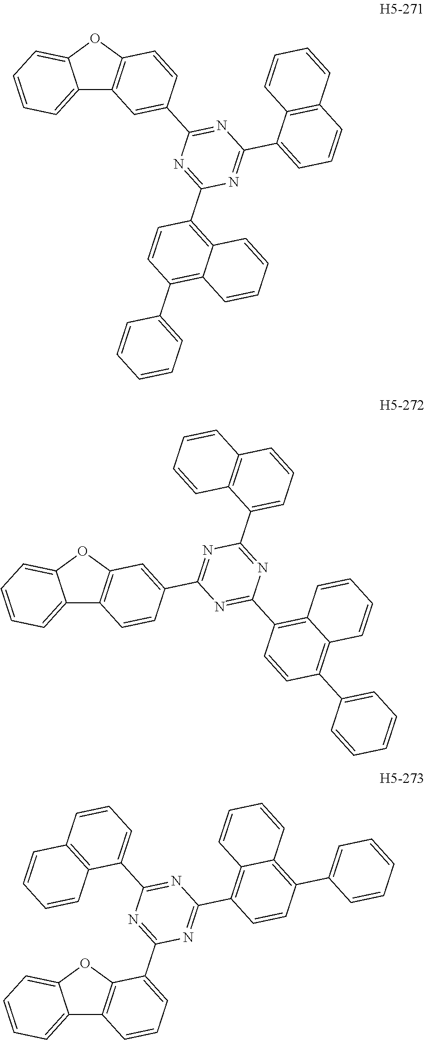 [SMILES: c1ccc(-c2ccc(-c3nc(-c4ccc5oc6ccccc6c5c4)nc(-c4cccc5ccccc45)n3)c3ccccc23)cc1 ; c1ccc(-c2ccc(-c3nc(-c4ccc5c(c4)oc4ccccc45)nc(-c4cccc5ccccc45)n3)c3ccccc23)cc1 ; c1ccc(-c2ccc(-c3nc(-c4cccc5ccccc45)nc(-c4cccc5c4oc4ccccc45)n3)c3ccccc23)cc1]

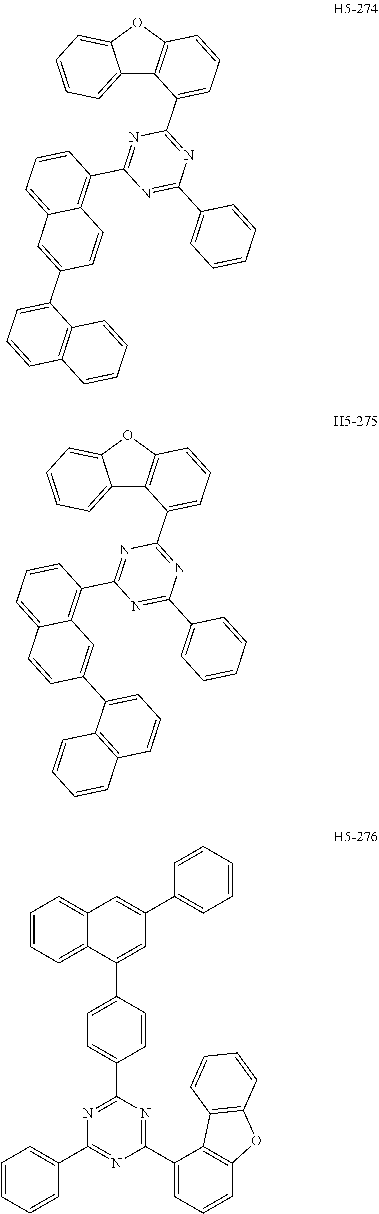 [SMILES: c1ccc(-c2nc(-c3cccc4cc(-c5cccc6ccccc56)ccc34)nc(-c3cccc4oc5ccccc5c34)n2)cc1 ; c1ccc(-c2nc(-c3cccc4ccc(-c5cccc6ccccc56)cc34)nc(-c3cccc4oc5ccccc5c34)n2)cc1 ; c1ccc(-c2cc(-c3ccc(-c4nc(-c5ccccc5)nc(-c5cccc6oc7ccccc7c56)n4)cc3)c3ccccc3c2)cc1]

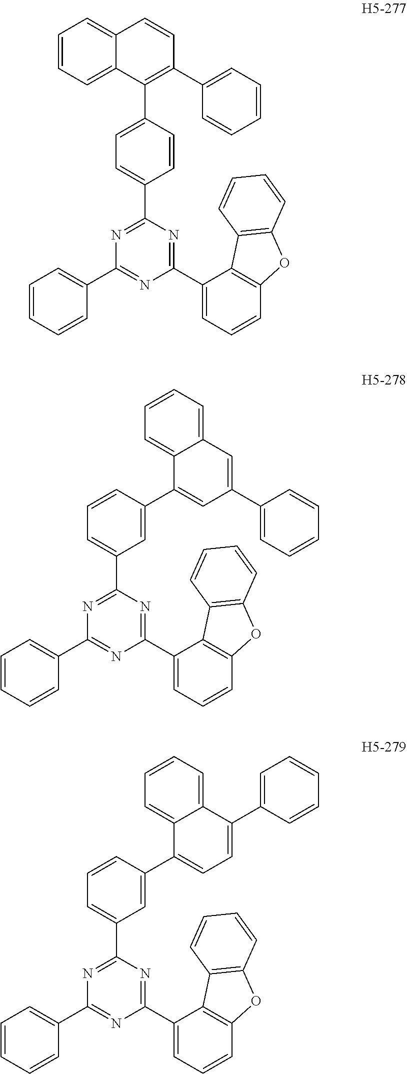 [SMILES: c1ccc(-c2nc(-c3ccc(-c4c(-c5ccccc5)ccc5ccccc45)cc3)nc(-c3cccc4oc5ccccc5c34)n2)cc1 ; c1ccc(-c2cc(-c3cccc(-c4nc(-c5ccccc5)nc(-c5cccc6oc7ccccc7c56)n4)c3)c3ccccc3c2)cc1 ; c1ccc(-c2nc(-c3cccc(-c4ccc(-c5ccccc5)c5ccccc45)c3)nc(-c3cccc4oc5ccccc5c34)n2)cc1]

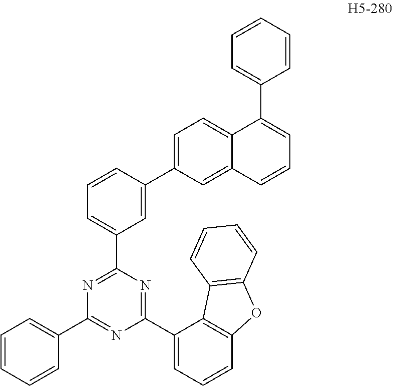 [SMILES: c1ccc(-c2nc(-c3cccc(-c4ccc5c(-c6ccccc6)cccc5c4)c3)nc(-c3cccc4oc5ccccc5c34)n2)cc1]

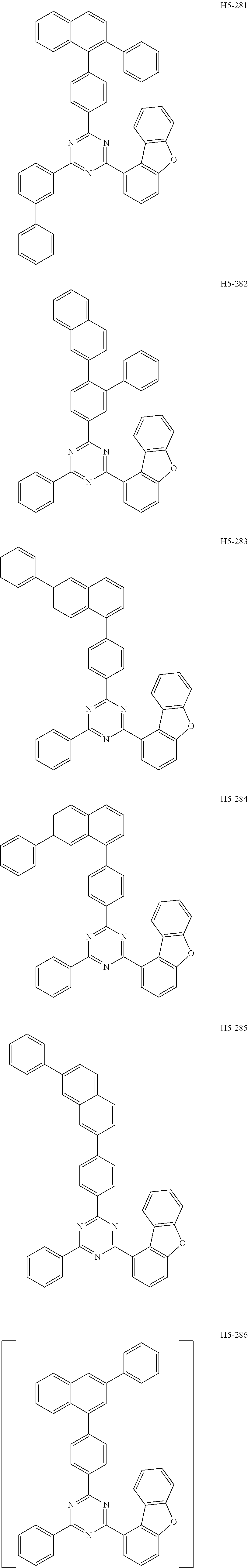

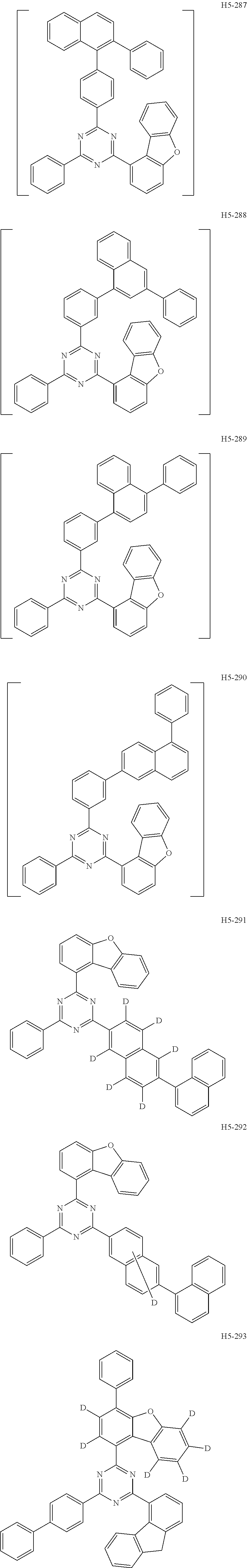

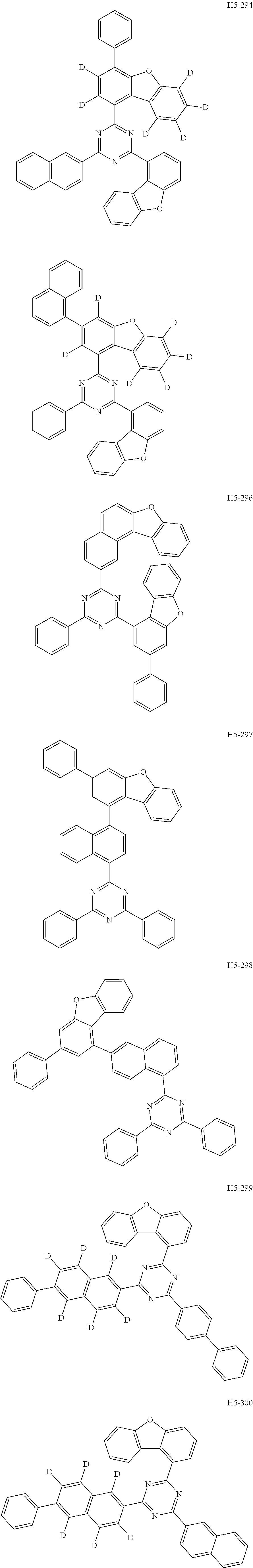

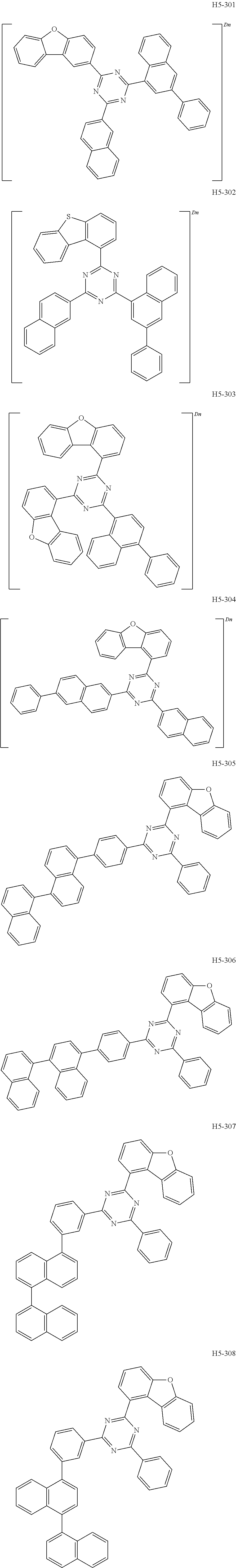

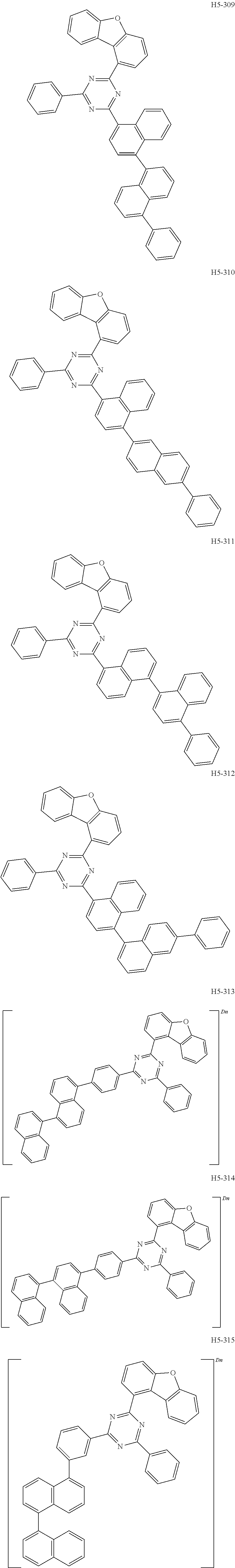

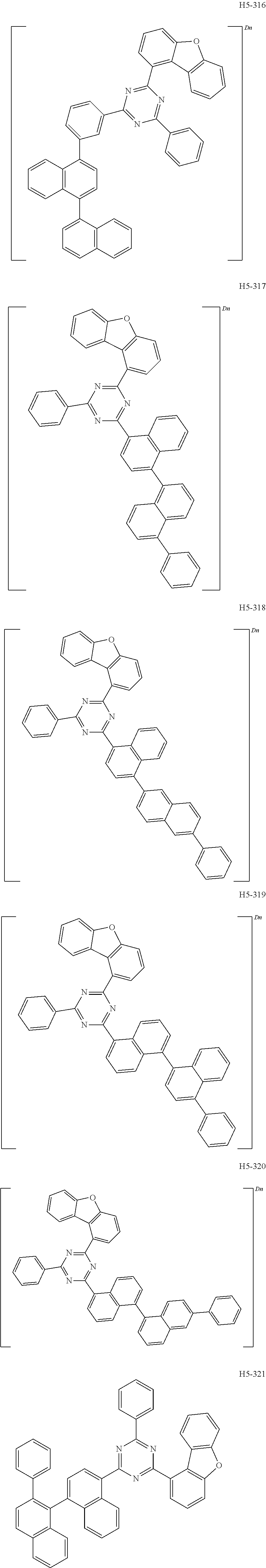

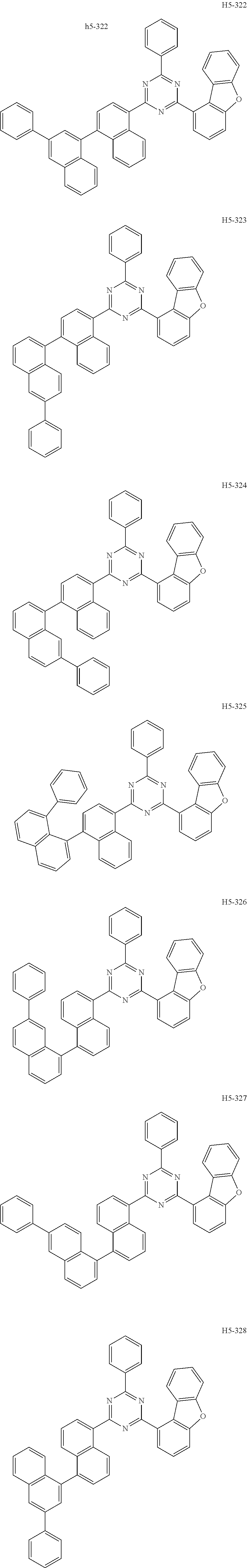

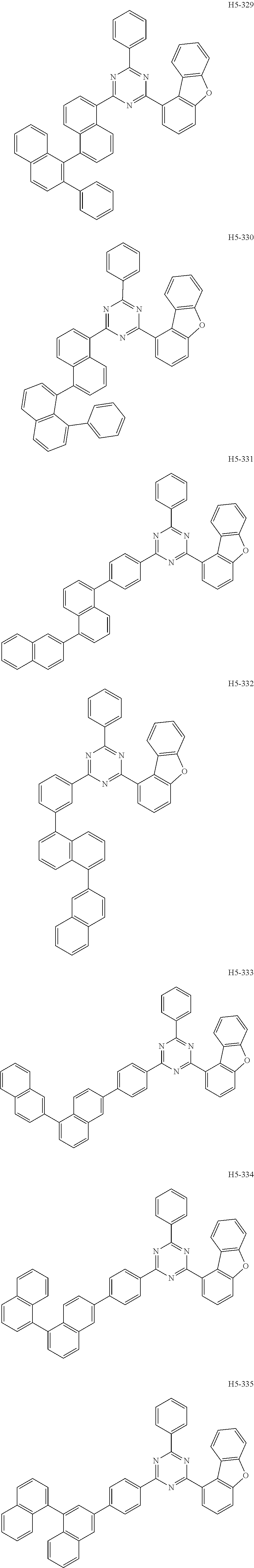

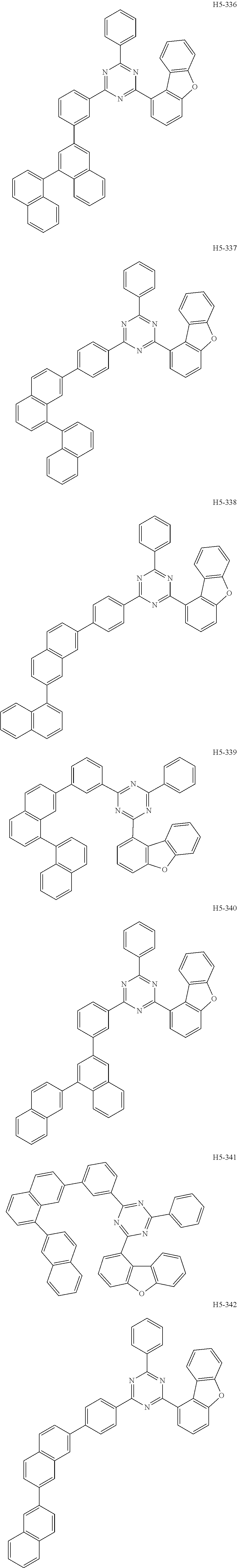

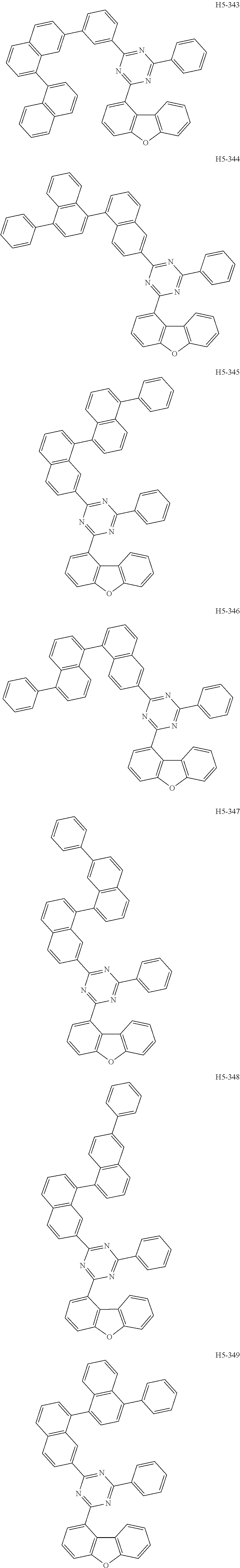

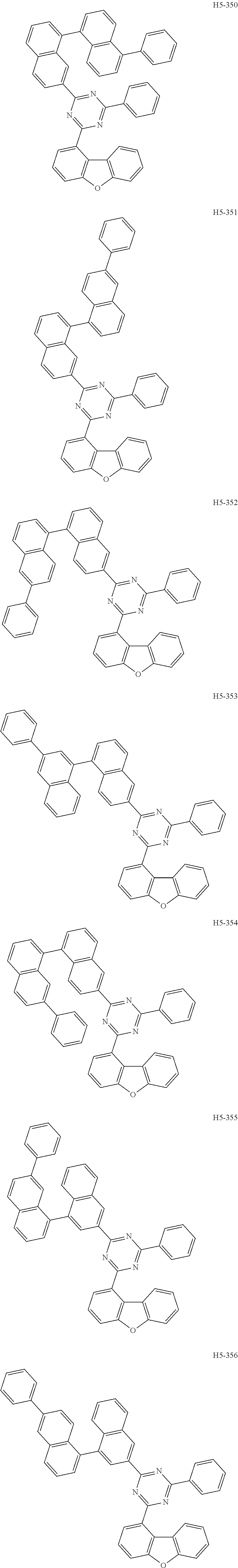

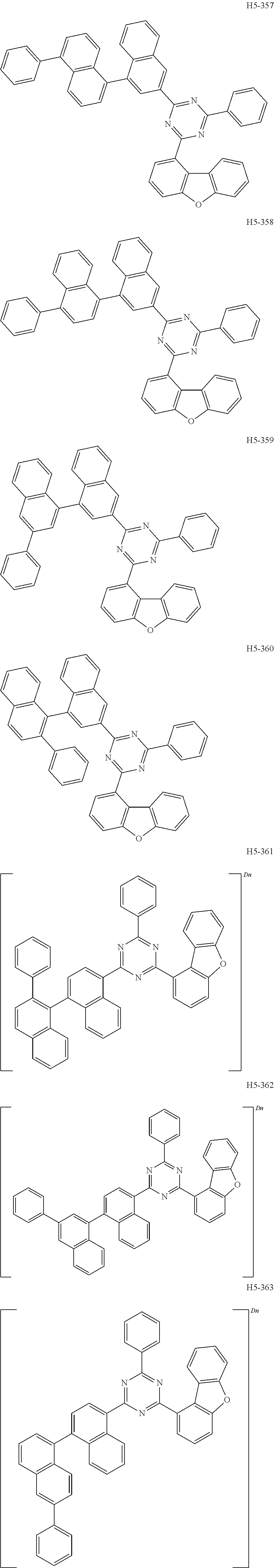

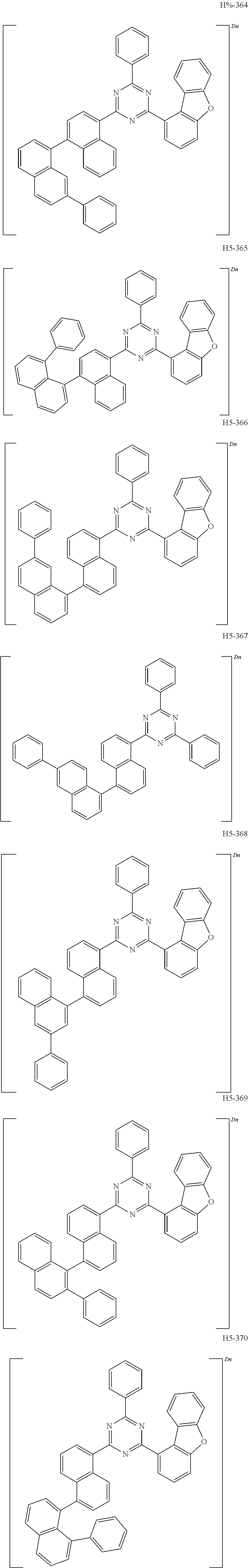

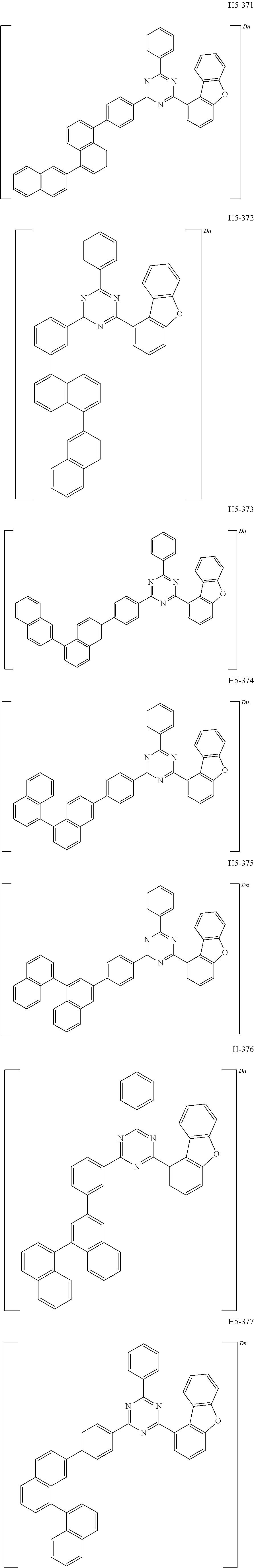

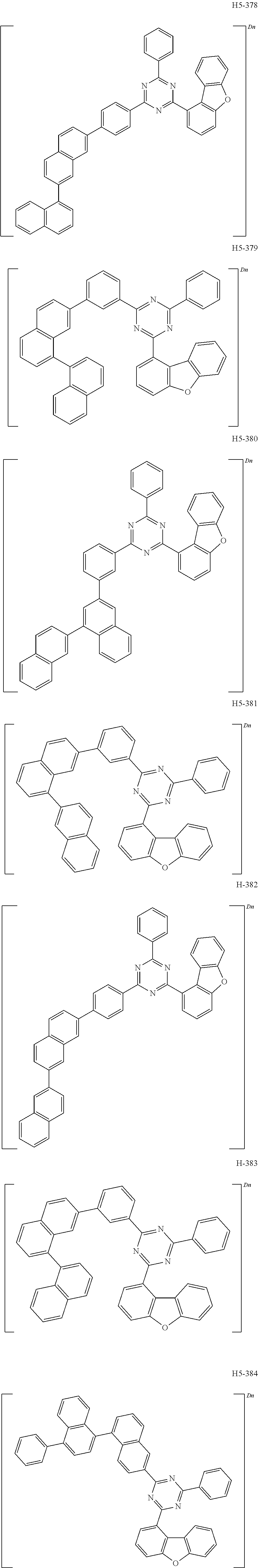

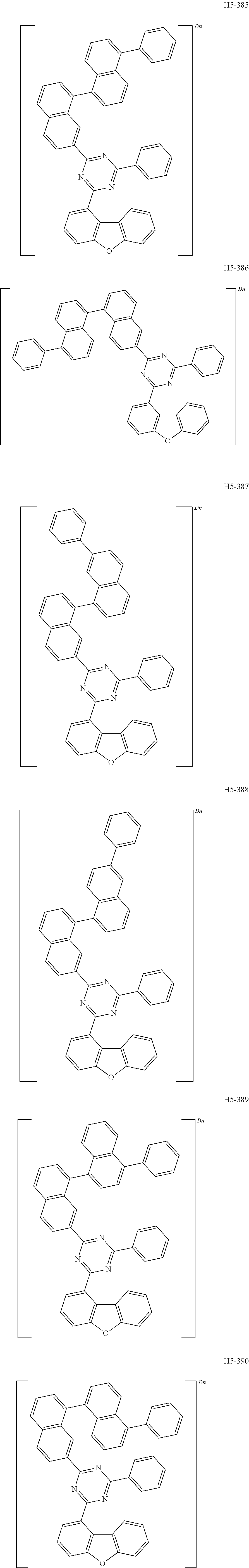

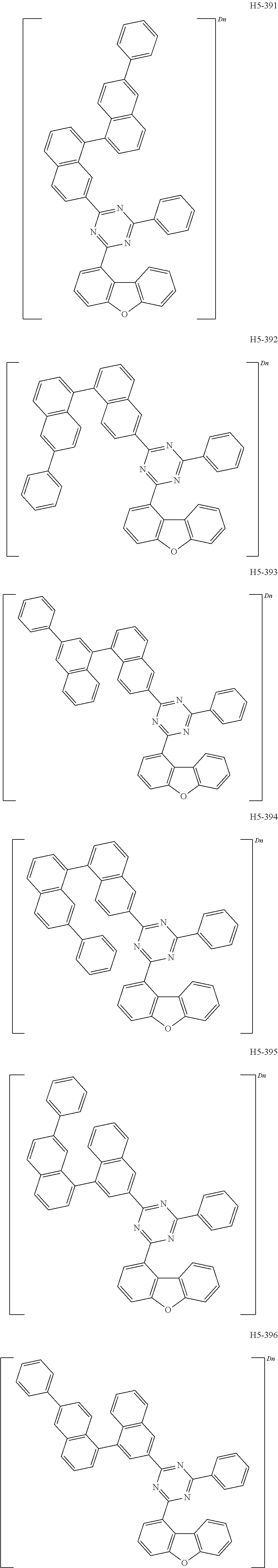

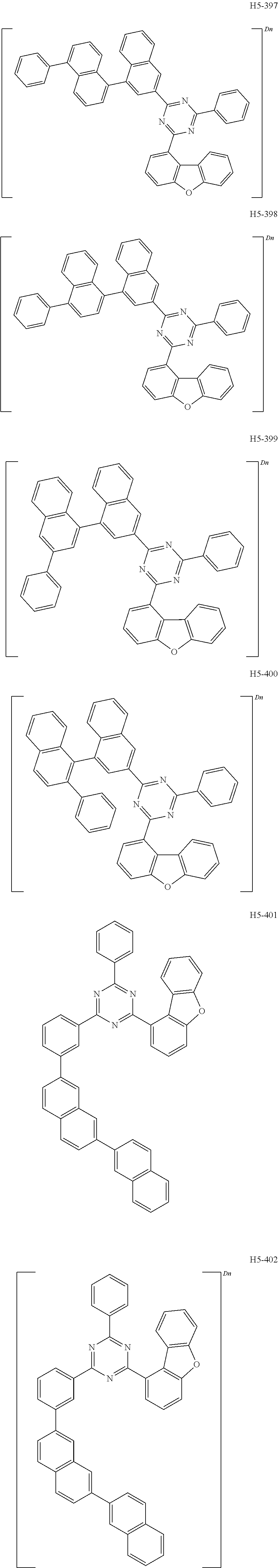

In the compounds above, Dₙ means that n number of hydrogens is replaced with deuterium, wherein n is an integer of 1 or more, and the upper limit of n is determined according to the number of hydrogens that may be substituted for each compound.

The compound represented by Formula 3 according to the present disclosure can be prepared by referring to synthetic methods known to those skilled in the art, for example, synthetic methods disclosed in Korean Patent Application Laid-Open Nos. 2022-0051794, 2021-0124018, and 2021-010943, etc. but is not limited thereto.

According to another embodiment, the present disclosure provides an organic electroluminescent compound represented by the following Formula 1-1.

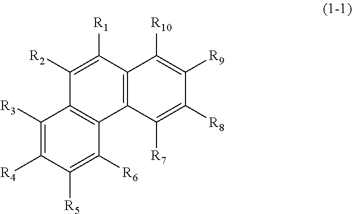

In Formula 1-1,

R₁ to R₁₀ each independently represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring of (C3-C30) aliphatic ring and (C6-C30) aromatic ring, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or *-L₁-N(Ar₁)(Ar₂); provided that at least one of R₁ to R₁₀ is *-L₁-N(Ar₁)(Ar₂);

L₁ represents a single bond, a substituted or unsubstituted (C6-C30)arylene, or a substituted or unsubstituted (3- to 30-membered)heteroarylene; and

Ar₁ and Ar₂ each independently represent a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl;
provided that if R₁, R₂, R₄, R₅, R₈, or R₉ is *-L₁-N(Ar₁)(Ar₂), the following conditions are satisfied.
(1) if R₅ or R₈ is *-L₁-N(Ar₁)(Ar₂), at least one of Ar₁ and Ar₂ is a phenyl unsubstituted or substituted with deuterium;
(2) if R₁ or R₂ is *-L₁-N(Ar₁)(Ar₂), at least one of Ar₁ and Ar₂ is a substituted or unsubstituted dibenzofuranyl or a substituted or unsubstituted carbazolyl; or all of Art and Ar₂ are a substituted or unsubstituted (3- to 30-membered)heteroaryl; and
(3) if R₄ or Reis *-L₁-N(Ar₁)(Ar₂), all of Ar₁ and Ar₂ are a substituted or unsubstituted (3- to 30-membered)heteroaryl; or at least one of Art and Ar₂ is a substituted or unsubstituted carbazolyl.

According to one embodiment, the organic electroluminescent compound represented by Formula 1-1 may be more specifically illustrated by the following compounds, but is not limited thereto:

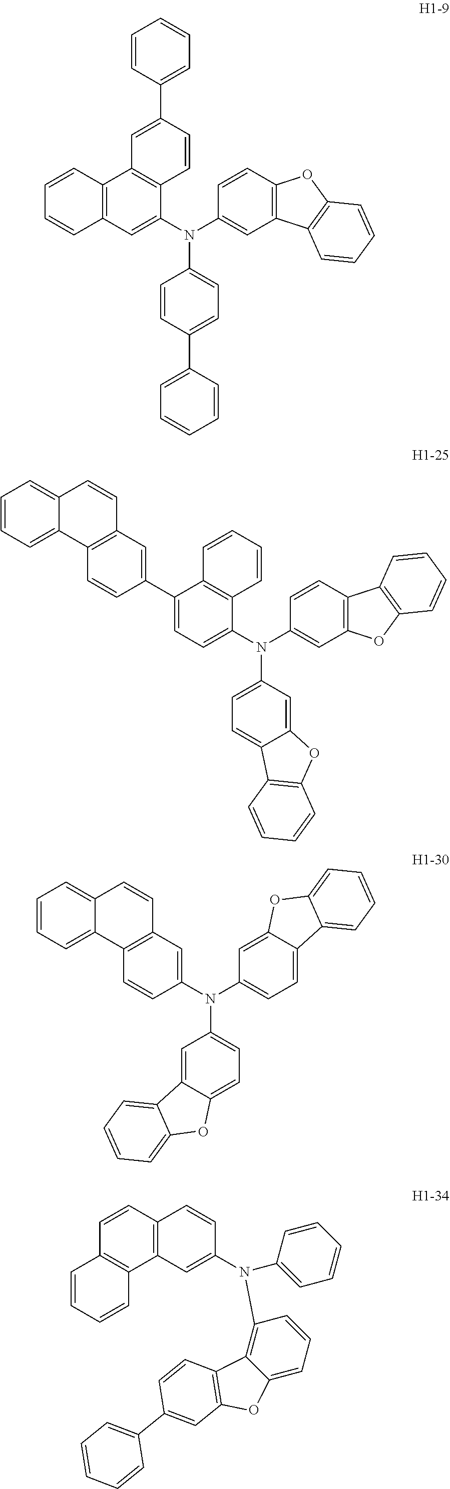

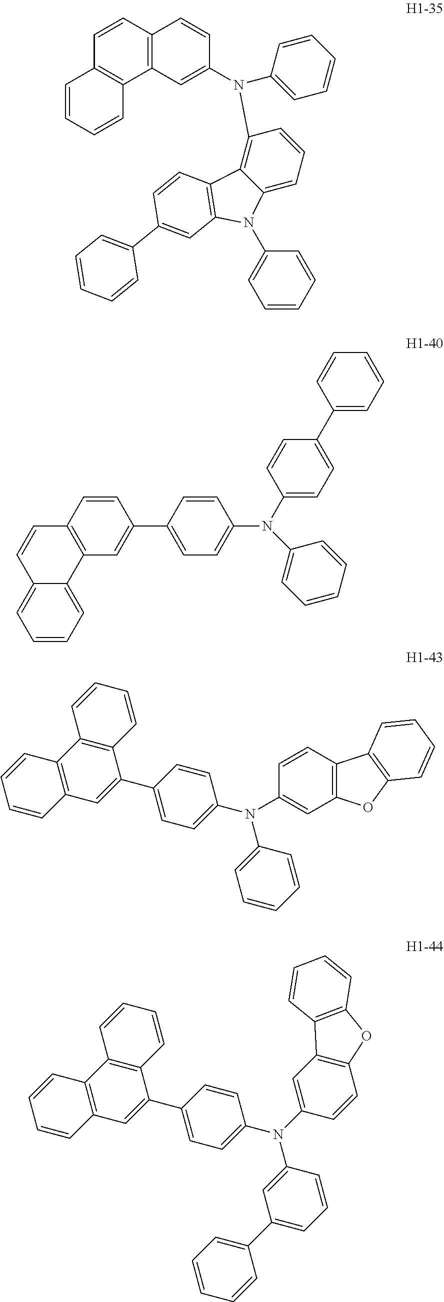 [SMILES: c1ccc(-c2ccc3c4c(N(c5ccccc5)c5ccc6ccc7ccccc7c6c5)cccc4n(-c4ccccc4)c3c2)cc1 ; c1ccc(-c2ccc(N(c3ccccc3)c3ccc(-c4ccc5ccc6ccccc6c5c4)cc3)cc2)cc1 ; c1ccc(N(c2ccc(-c3cc4ccccc4c4ccccc34)cc2)c2ccc3c(c2)oc2ccccc23)cc1 ; c1ccc(-c2cccc(N(c3ccc(-c4cc5ccccc5c5ccccc45)cc3)c3ccc4oc5ccccc5c4c3)c2)cc1]

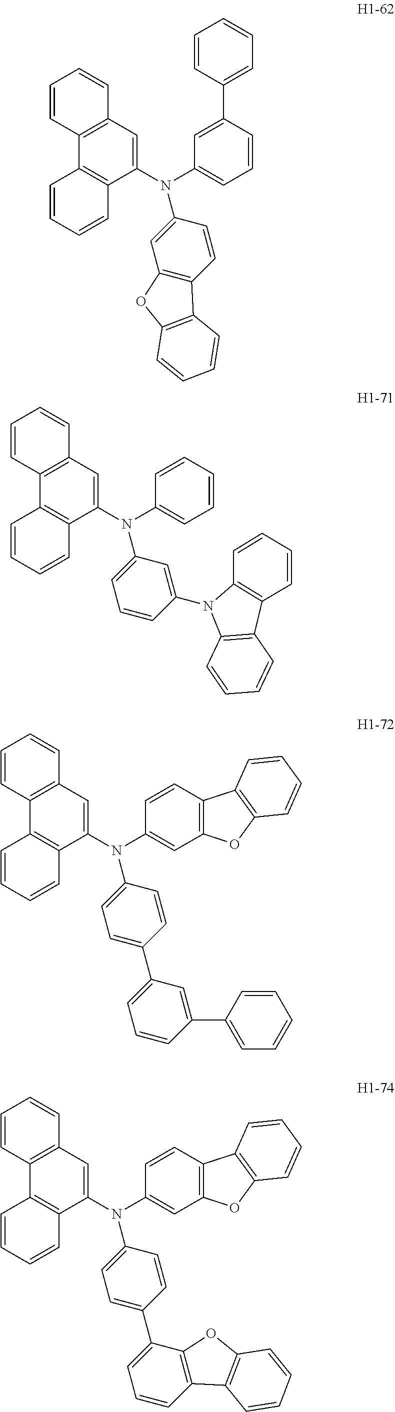 [SMILES: c1ccc(-c2cccc(N(c3ccc4c(c3)oc3ccccc34)c3cc4ccccc4c4ccccc34)c2)cc1 ; c1ccc(N(c2cccc(-n3c4ccccc4c4ccccc43)c2)c2cc3ccccc3c3ccccc23)cc1 ; c1ccc(-c2cccc(-c3ccc(N(c4ccc5c(c4)oc4ccccc45)c4cc5ccccc5c5ccccc45)cc3)c2)cc1 ; c1ccc2c(c1)cc(N(c1ccc(-c3cccc4c3oc3ccccc34)cc1)c1ccc3c(c1)oc1ccccc13)c1ccccc12]

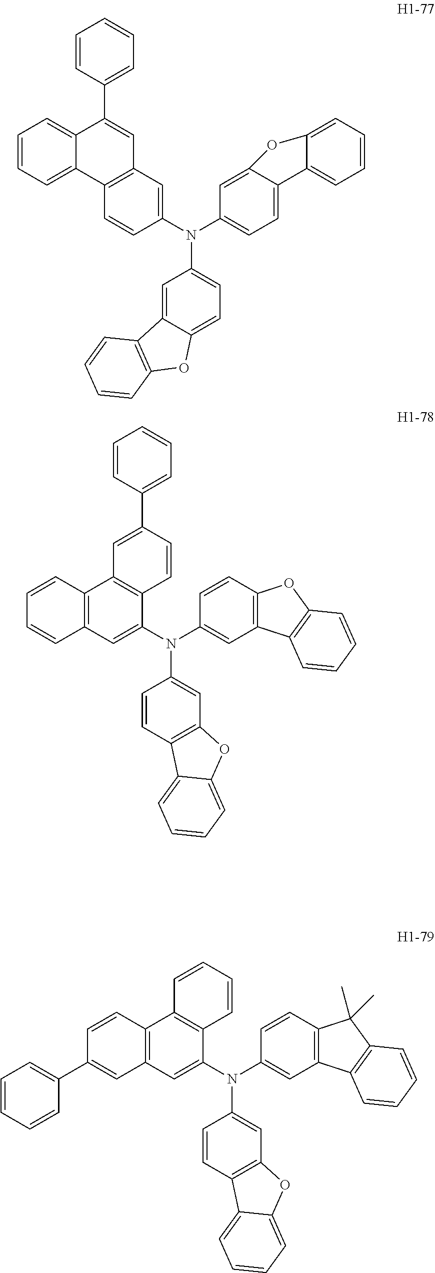

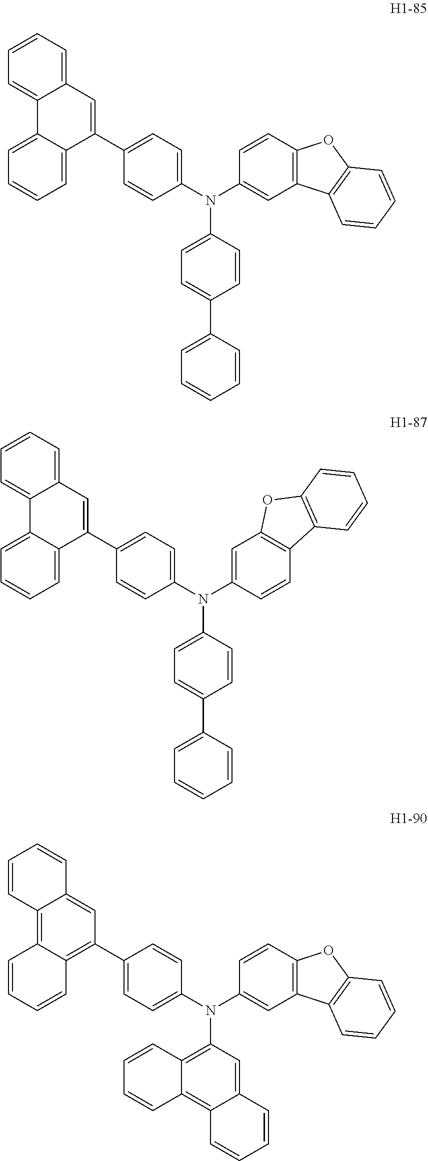 [SMILES: c1ccc(-c2ccc(N(c3ccc(-c4cc5ccccc5c5ccccc45)cc3)c3ccc4oc5ccccc5c4c3)cc2)cc1 ; c1ccc(-c2ccc(N(c3ccc(-c4cc5ccccc5c5ccccc45)cc3)c3ccc4c(c3)oc3ccccc34)cc2)cc1 ; c1ccc2c(c1)cc(-c1ccc(N(c3ccc4oc5ccccc5c4c3)c3cc4ccccc4c4ccccc34)cc1)c1ccccc12]

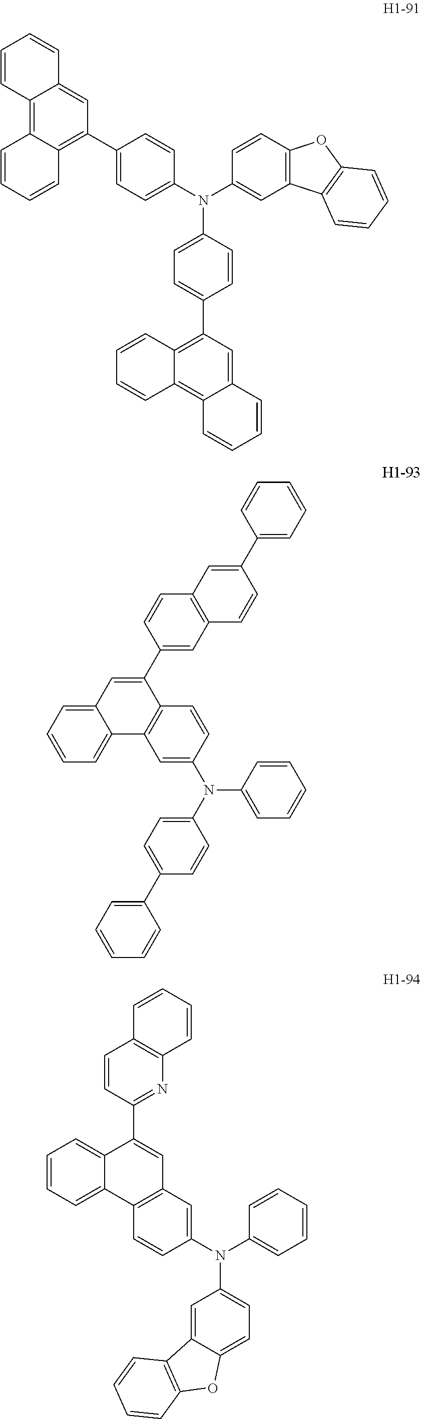

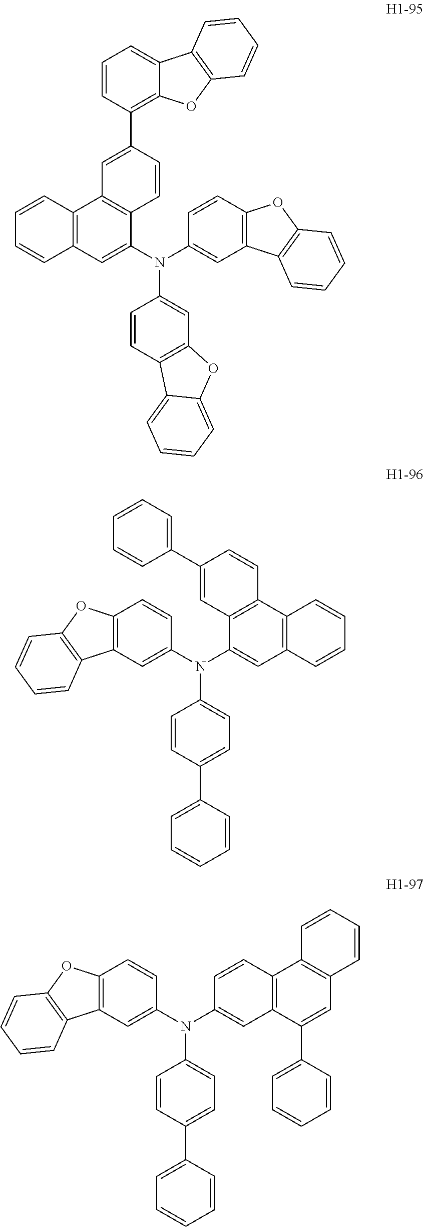 [SMILES: c1ccc2c(c1)cc(N(c1ccc3c(c1)oc1ccccc13)c1ccc3oc4ccccc4c3c1)c1ccc(-c3cccc4c3oc3ccccc34)cc12 ; c1ccc(-c2ccc(N(c3ccc4oc5ccccc5c4c3)c3cc4ccccc4c4ccc(-c5ccccc5)cc34)cc2)cc1 ; c1ccc(-c2ccc(N(c3ccc4c(c3)c(-c3ccccc3)cc3ccccc34)c3ccc4oc5ccccc5c4c3)cc2)cc1]

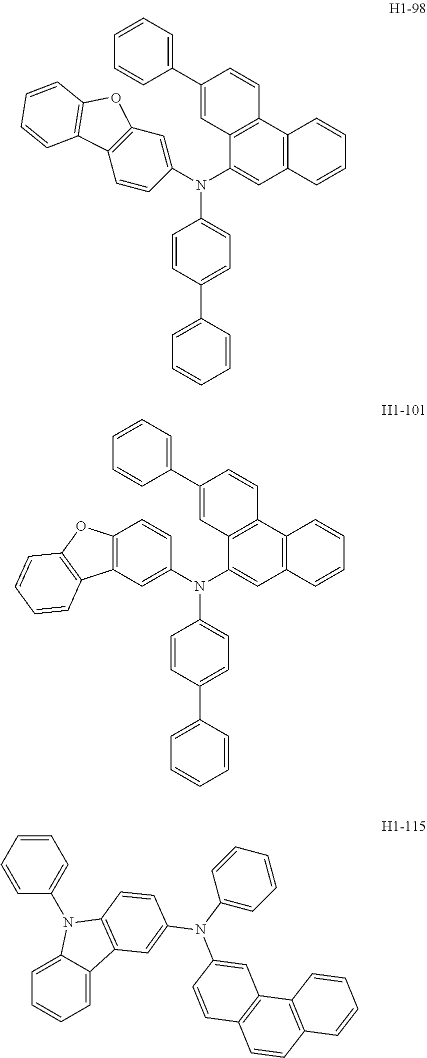 [SMILES: c1ccc(-c2ccc(N(c3ccc4c(c3)oc3ccccc34)c3cc4ccccc4c4ccc(-c5ccccc5)cc34)cc2)cc1 ; c1ccc(-c2ccc(N(c3ccc4oc5ccccc5c4c3)c3cc4ccccc4c4ccc(-c5ccccc5)cc34)cc2)cc1 ; c1ccc(N(c2ccc3ccc4ccccc4c3c2)c2ccc3c(c2)c2ccccc2n3-c2ccccc2)cc1]

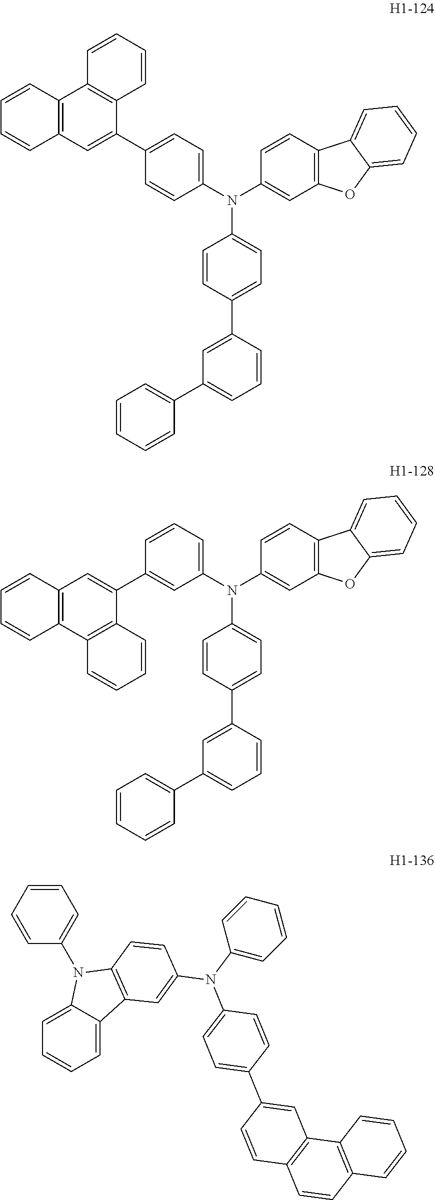 [SMILES: c1ccc(-c2cccc(-c3ccc(N(c4ccc(-c5cc6ccccc6c6ccccc56)cc4)c4ccc5c(c4)oc4ccccc45)cc3)c2)cc1 ; c1ccc(-c2cccc(-c3ccc(N(c4cccc(-c5cc6ccccc6c6ccccc56)c4)c4ccc5c(c4)oc4ccccc45)cc3)c2)cc1 ; c1ccc(N(c2ccc(-c3ccc4ccc5ccccc5c4c3)cc2)c2ccc3c(c2)c2ccccc2n3-c2ccccc2)cc1]

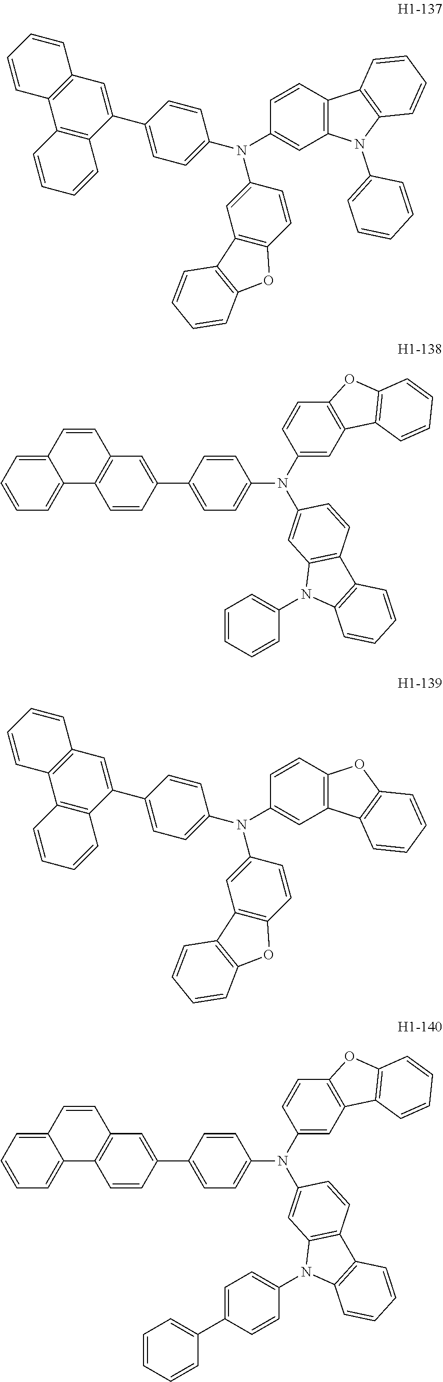 [SMILES: c1ccc(-n2c3ccccc3c3ccc(N(c4ccc(-c5cc6ccccc6c6ccccc56)cc4)c4ccc5oc6ccccc6c5c4)cc32)cc1 ; c1ccc(-n2c3ccccc3c3ccc(N(c4ccc(-c5ccc6c(ccc7ccccc76)c5)cc4)c4ccc5oc6ccccc6c5c4)cc32)cc1 ; c1ccc2c(c1)cc(-c1ccc(N(c3ccc4oc5ccccc5c4c3)c3ccc4oc5ccccc5c4c3)cc1)c1ccccc12 ; c1ccc(-c2ccc(-n3c4ccccc4c4ccc(N(c5ccc(-c6ccc7c(ccc8ccccc87)c6)cc5)c5ccc6oc7ccccc7c6c5)cc43)cc2)cc1]

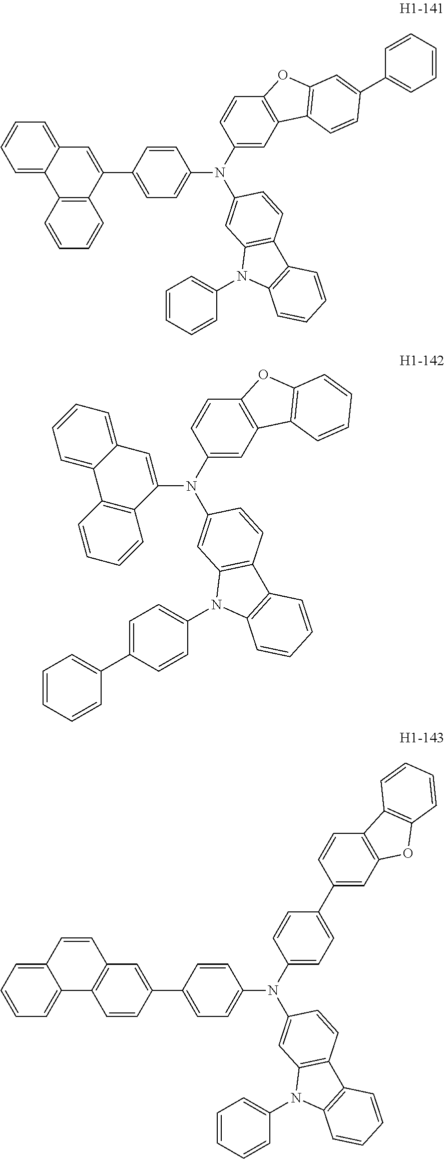 [SMILES: c1ccc(-c2ccc3c(c2)oc2ccc(N(c4ccc(-c5cc6ccccc6c6ccccc56)cc4)c4ccc5c6ccccc6n(-c6ccccc6)c5c4)cc23)cc1 ; c1ccc(-c2ccc(-n3c4ccccc4c4ccc(N(c5ccc6oc7ccccc7c6c5)c5cc6ccccc6c6ccccc56)cc43)cc2)cc1 ; c1ccc(-n2c3ccccc3c3ccc(N(c4ccc(-c5ccc6c(ccc7ccccc76)c5)cc4)c4ccc(-c5ccc6c(c5)oc5ccccc56)cc4)cc32)cc1]

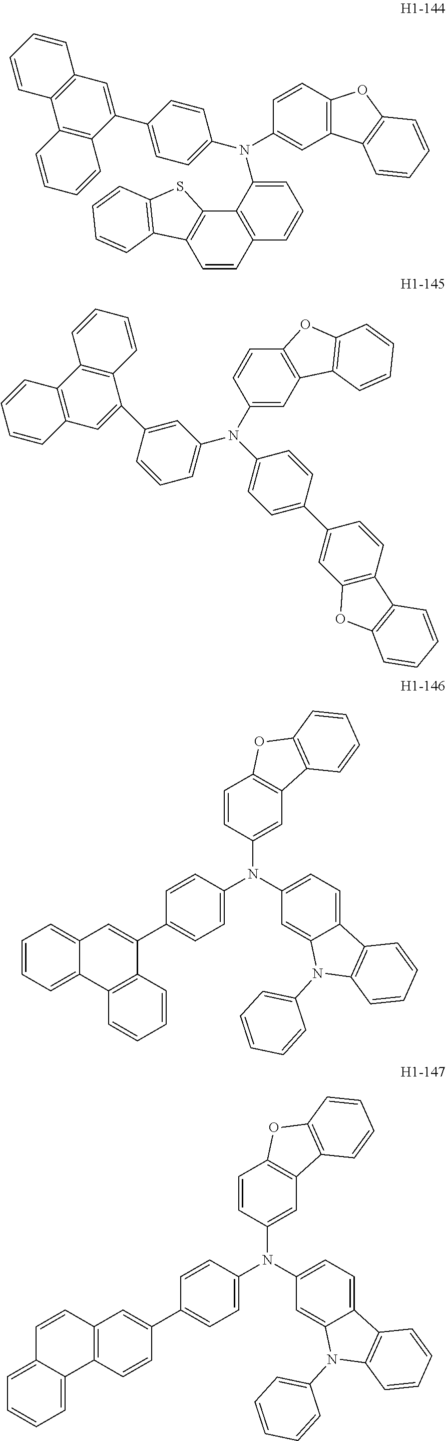 [SMILES: c1ccc2c(c1)cc(-c1ccc(N(c3ccc4oc5ccccc5c4c3)c3cccc4ccc5c6ccccc6sc5c34)cc1)c1ccccc12 ; c1cc(-c2cc3ccccc3c3ccccc23)cc(N(c2ccc(-c3ccc4c(c3)oc3ccccc34)cc2)c2ccc3oc4ccccc4c3c2)c1 ; c1ccc(-n2c3ccccc3c3ccc(N(c4ccc(-c5cc6ccccc6c6ccccc56)cc4)c4ccc5oc6ccccc6c5c4)cc32)cc1 ; c1ccc(-n2c3ccccc3c3ccc(N(c4ccc(-c5ccc6c(ccc7ccccc76)c5)cc4)c4ccc5oc6ccccc6c5c4)cc32)cc1]

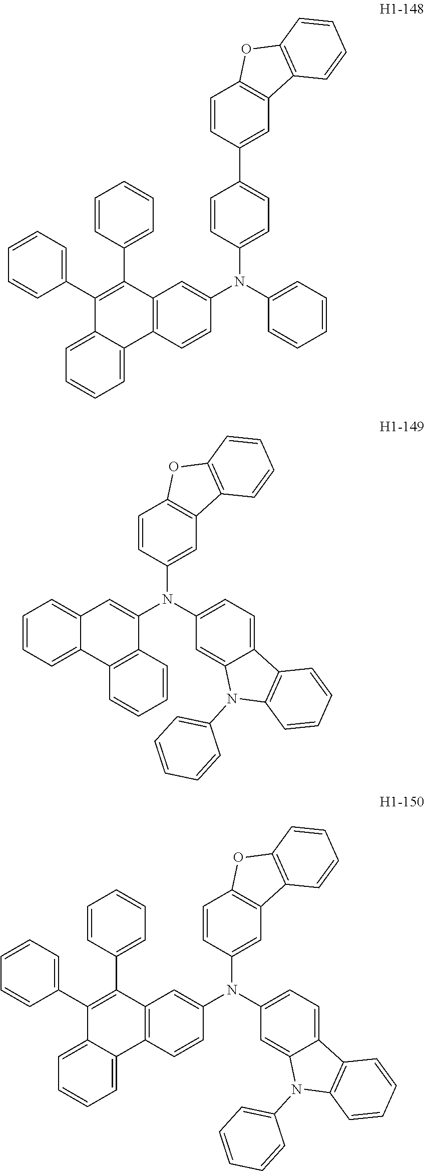 [SMILES: c1ccc(-c2c(-c3ccccc3)c3cc(N(c4ccccc4)c4ccc(-c5ccc6oc7ccccc7c6c5)cc4)ccc3c3ccccc23)cc1 ; c1ccc(-n2c3ccccc3c3ccc(N(c4ccc5oc6ccccc6c5c4)c4cc5ccccc5c5ccccc45)cc32)cc1 ; c1ccc(-c2c(-c3ccccc3)c3cc(N(c4ccc5oc6ccccc6c5c4)c4ccc5c6ccccc6n(-c6ccccc6)c5c4)ccc3c3ccccc23)cc1]

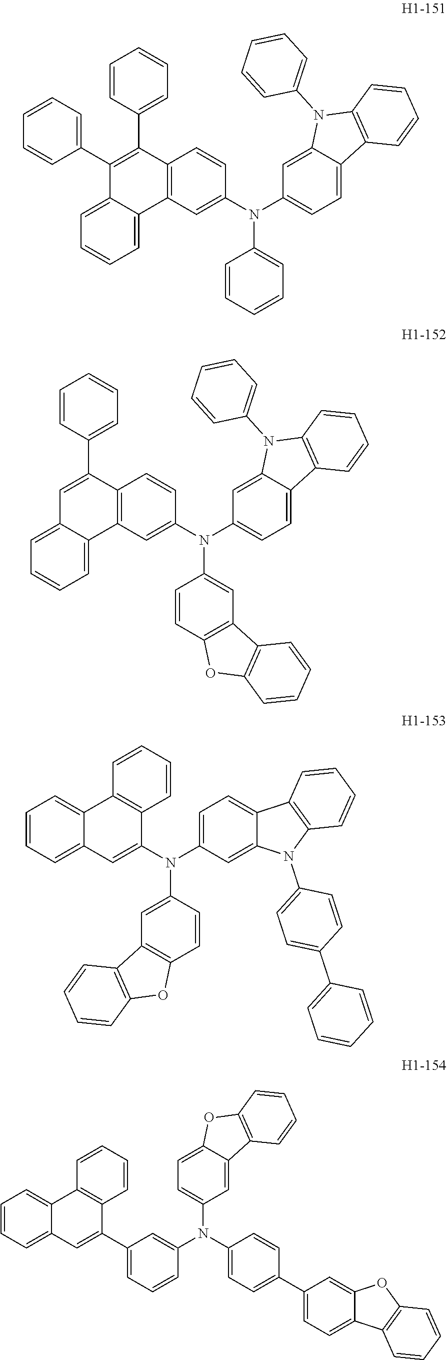 [SMILES: c1ccc(-c2c(-c3ccccc3)c3ccc(N(c4ccccc4)c4ccc5c6ccccc6n(-c6ccccc6)c5c4)cc3c3ccccc23)cc1 ; c1ccc(-c2cc3ccccc3c3cc(N(c4ccc5oc6ccccc6c5c4)c4ccc5c6ccccc6n(-c6ccccc6)c5c4)ccc23)cc1 ; c1ccc(-c2ccc(-n3c4ccccc4c4ccc(N(c5ccc6oc7ccccc7c6c5)c5cc6ccccc6c6ccccc56)cc43)cc2)cc1 ; c1cc(-c2cc3ccccc3c3ccccc23)cc(N(c2ccc(-c3ccc4c(c3)oc3ccccc34)cc2)c2ccc3oc4ccccc4c3c2)c1]

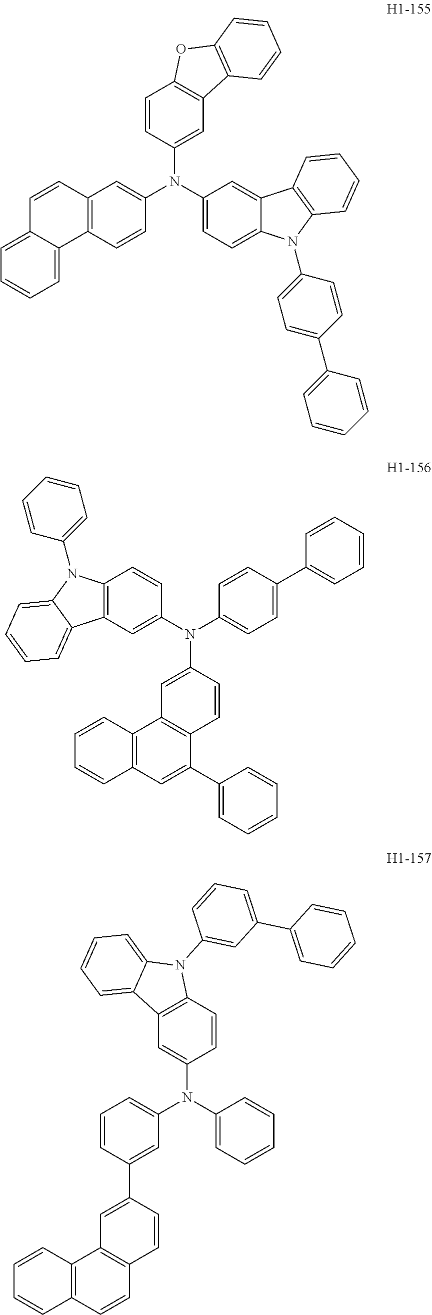 [SMILES: c1ccc(-c2ccc(-n3c4ccccc4c4cc(N(c5ccc6c(ccc7ccccc76)c5)c5ccc6oc7ccccc7c6c5)ccc43)cc2)cc1 ; c1ccc(-c2ccc(N(c3ccc4c(-c5ccccc5)cc5ccccc5c4c3)c3ccc4c(c3)c3ccccc3n4-c3ccccc3)cc2)cc1 ; c1ccc(-c2cccc(-n3c4ccccc4c4cc(N(c5ccccc5)c5cccc(-c6ccc7ccc8ccccc8c7c6)c5)ccc43)c2)cc1]

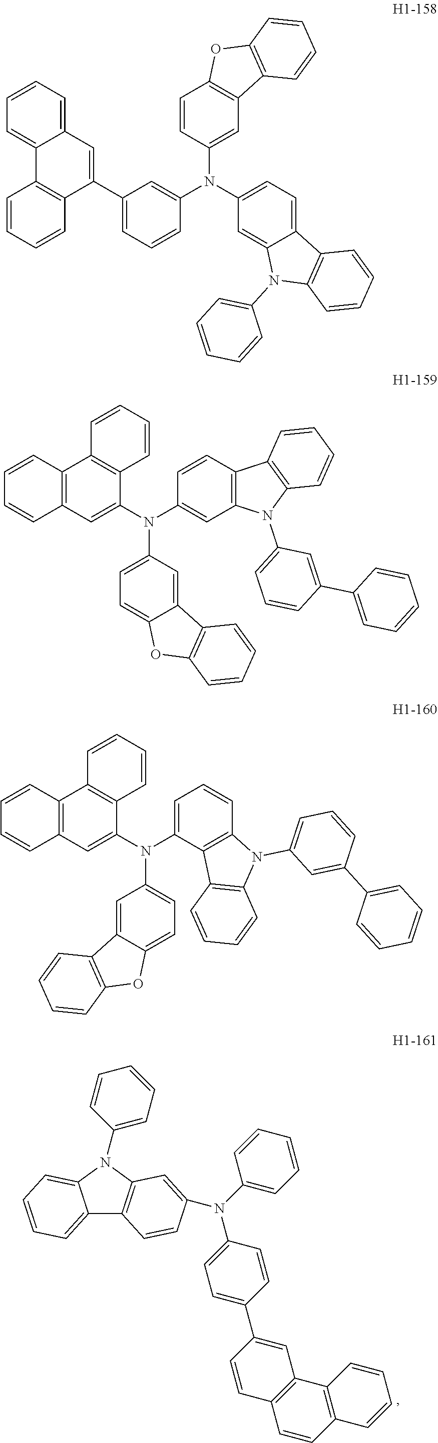 [SMILES: c1ccc(-n2c3ccccc3c3ccc(N(c4cccc(-c5cc6ccccc6c6ccccc56)c4)c4ccc5oc6ccccc6c5c4)cc32)cc1 ; c1ccc(-c2cccc(-n3c4ccccc4c4ccc(N(c5ccc6oc7ccccc7c6c5)c5cc6ccccc6c6ccccc56)cc43)c2)cc1 ; c1ccc(-c2cccc(-n3c4ccccc4c4c(N(c5ccc6oc7ccccc7c6c5)c5cc6ccccc6c6ccccc56)cccc43)c2)cc1 ; c1ccc(N(c2ccc(-c3ccc4ccc5ccccc5c4c3)cc2)c2ccc3c4ccccc4n(-c4ccccc4)c3c2)cc1]

According to another embodiment, the present disclosure provides an organic electroluminescent compound represented by the following Formula 3′.

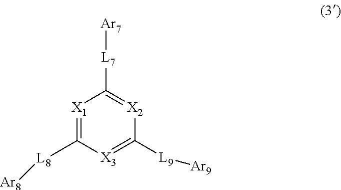

In Formula 3′,
X₁ to X₃ each independently represent N or CR₁₁; provided that at least one of X₁ to X₃ is N;
R₁₁ represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring of (C3-C30) aliphatic ring and (C6-C30) aromatic ring;
L₇ to L₉ each independently represent a single bond, a substituted or unsubstituted (C6-C30)arylene, or a substituted or unsubstituted (3- to 30-membered)heteroarylene; and
Ar₇ to Ar₉ each independently represent a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring of (C3-C30) aliphatic ring and (C6-C30)aromatic ring, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₂₁-N(Ar₂₁)(Ar₂₂);
provided that at least one of Ar₇ to Ar₉ is Formula 2-2.

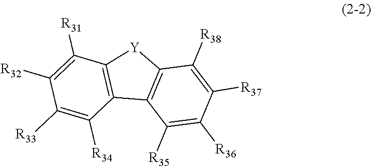

In Formula 2-2,
Y represents —O— or —S—; and
R₃₁ to R₃₈ each independently represent a connection position connected to L₇ to L₉ in Formula 3; or hydrogen, deuterium, a halogen, a cyano, a substituted or unsubstituted (C1-C30)alkyl, a substituted or unsubstituted (C6-C30)aryl, a substituted or unsubstituted (3-to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to the adjacent substituents to form a ring(s);
provided that if one of L₇ to L₉ is a phenylene unsubstituted or substituted with deuterium or cyano, at least one of Ar₇ to Ar₉ is a binaphthyl unsubstituted or substituted with deuterium or cyano, and if one of L₇ to L₉ is a naphthylene unsubstituted or substituted with deuterium or cyano, at least one of Ar₇ to Ar₉ is a phenylnaphthyl unsubstituted or substituted with deuterium or cyano.

In one embodiment, in Formula 3′, if one of L₇ to L₉ is phenylene substituted or unsubstituted with deuterium or cyano, at least one of Ar₇ to Ar₉ may be a substituent unsubstituted or substituted with deuterium or cyano as represented below.

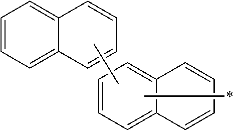

wherein * indicates a connection position connected to one of L₇ to L₉.

In one embodiment, in Formula 3′, if one of L₇ to L₉ is a naphthylene substituted or unsubstituted with deuterium or cyano, at least one of Ar₇ to Ar₉ may be a substituent unsubstituted or substituted with deuterium or cyano as represented below.

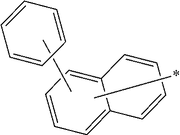

wherein * indicates a connection position connected to one of L₇ to L₉.

According to one embodiment, the compound represented by Formula 3′ can be more specifically exemplified as the following compounds, but is not limited thereto:

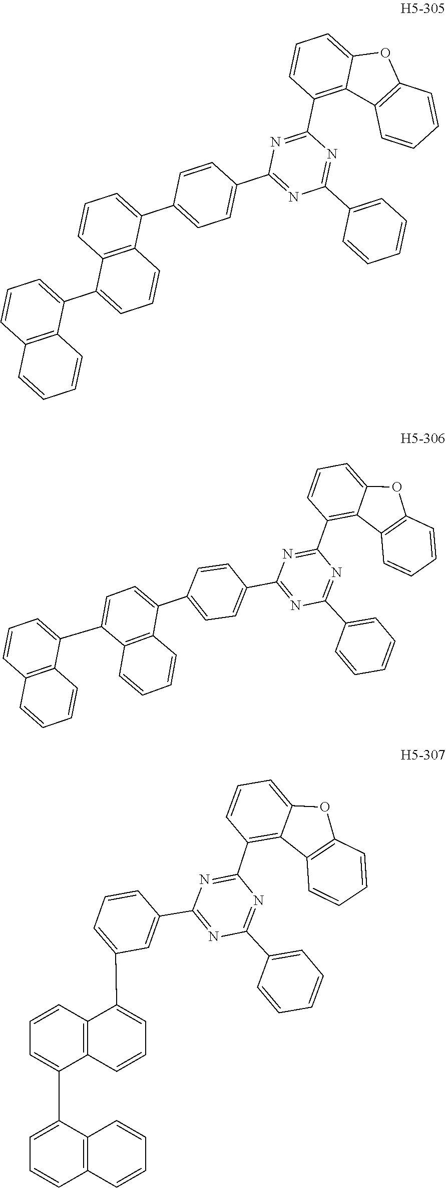 [SMILES: c1ccc(-c2nc(-c3ccc(-c4cccc5c(-c6cccc7ccccc67)cccc45)cc3)nc(-c3cccc4oc5ccccc5c34)n2)cc1 ; c1ccc(-c2nc(-c3ccc(-c4ccc(-c5cccc6ccccc56)c5ccccc45)cc3)nc(-c3cccc4oc5ccccc5c34)n2)cc1 ; c1ccc(-c2nc(-c3cccc(-c4cccc5c(-c6cccc7ccccc67)cccc45)c3)nc(-c3cccc4oc5ccccc5c34)n2)cc1]

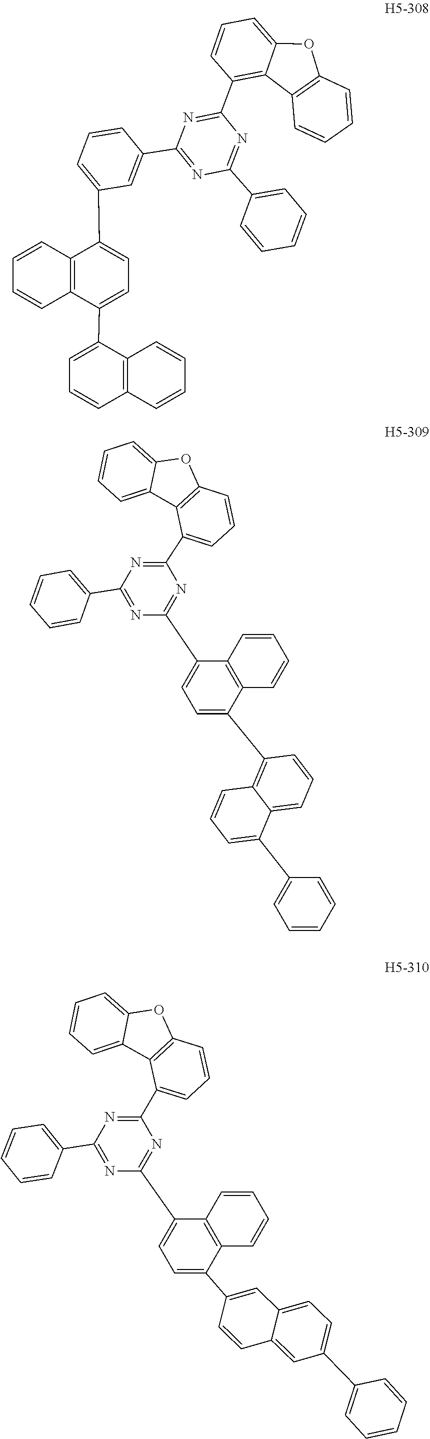

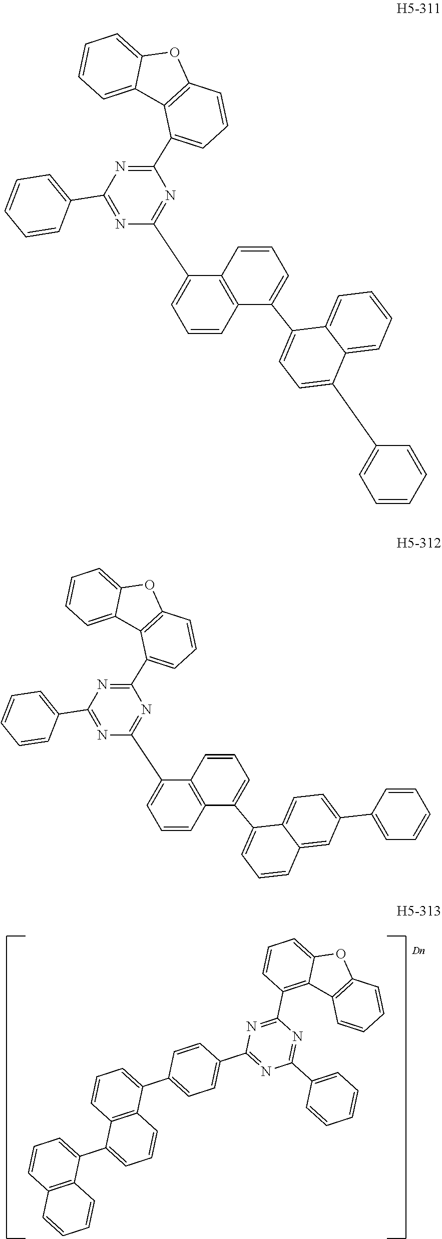

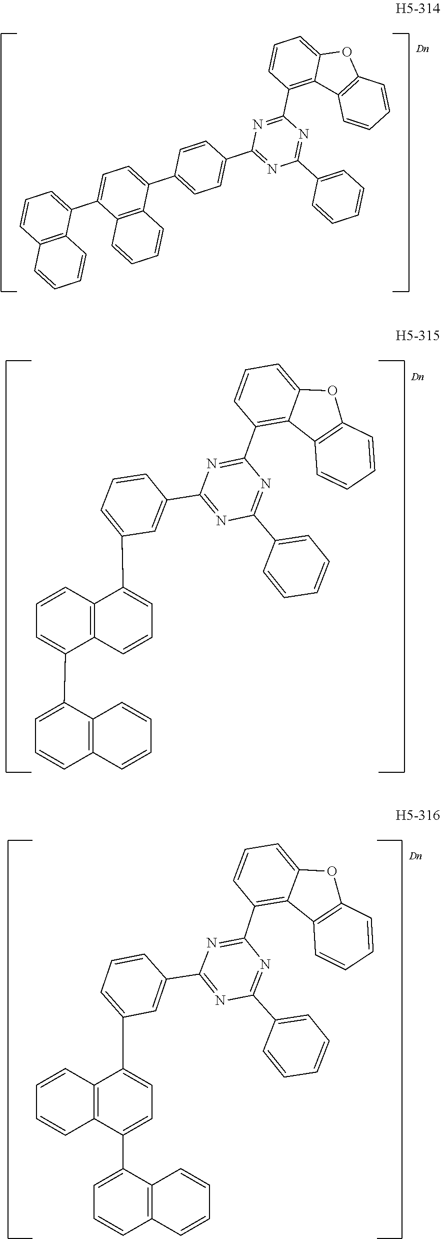

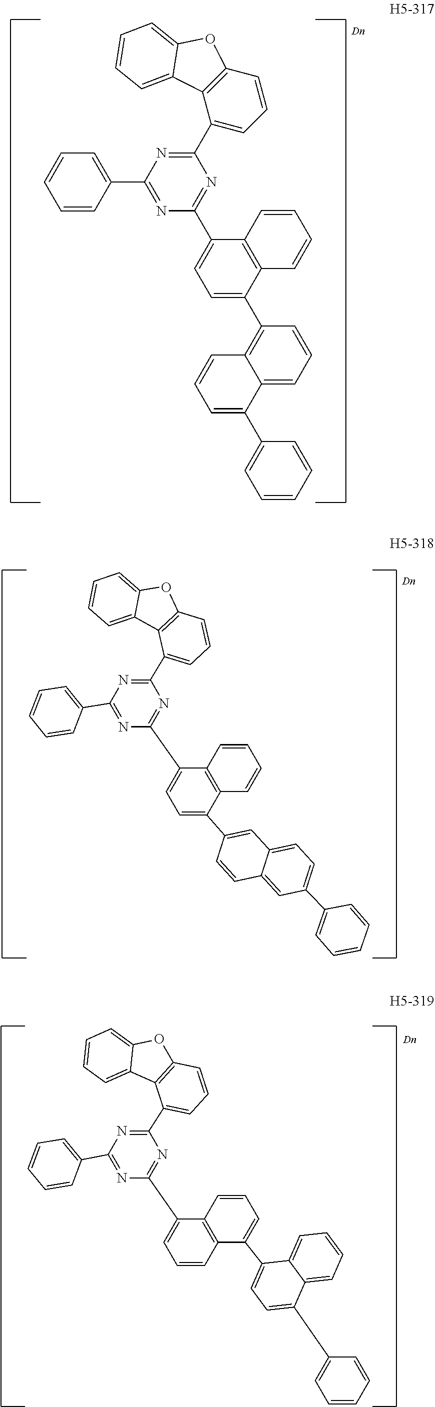

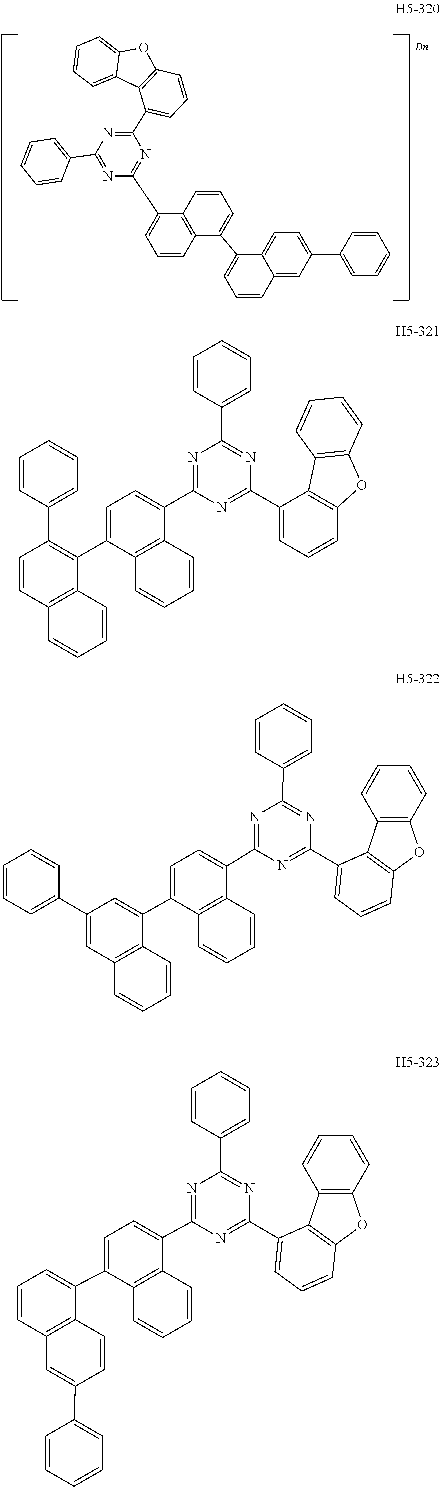

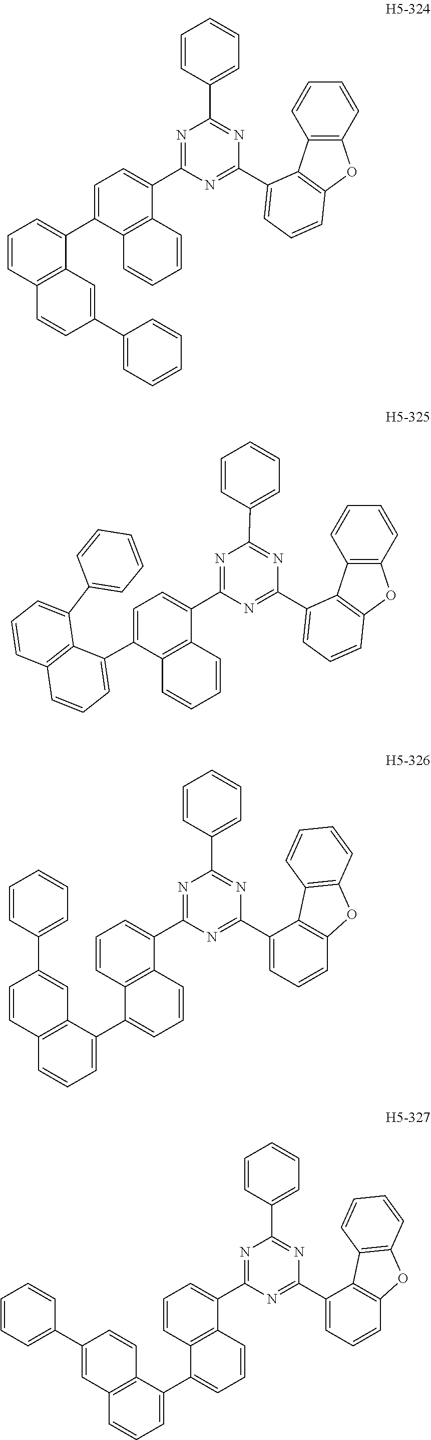 [SMILES: c1ccc(-c2ccc3cccc(-c4ccc(-c5nc(-c6ccccc6)nc(-c6cccc7oc8ccccc8c67)n5)c5ccccc45)c3c2)cc1 ; c1ccc(-c2nc(-c3ccc(-c4cccc5cccc(-c6ccccc6)c45)c4ccccc34)nc(-c3cccc4oc5ccccc5c34)n2)cc1 ; c1ccc(-c2ccc3cccc(-c4cccc5c(-c6nc(-c7ccccc7)nc(-c7cccc8oc9ccccc9c78)n6)cccc45)c3c2)cc1 ; c1ccc(-c2ccc3c(-c4cccc5c(-c6nc(-c7ccccc7)nc(-c7cccc8oc9ccccc9c78)n6)cccc45)cccc3c2)cc1]

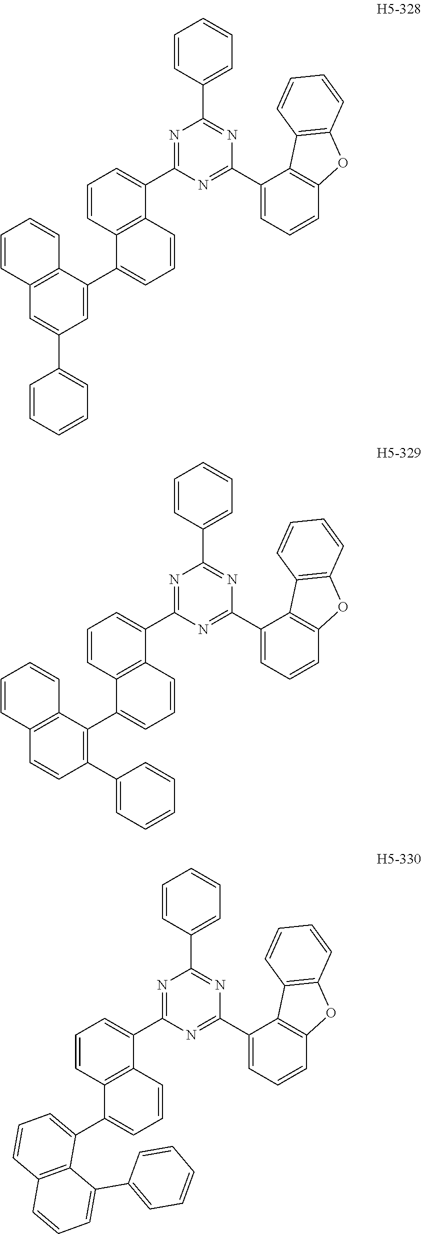 [SMILES: c1ccc(-c2cc(-c3cccc4c(-c5nc(-c6ccccc6)nc(-c6cccc7oc8ccccc8c67)n5)cccc34)c3ccccc3c2)cc1 ; c1ccc(-c2nc(-c3cccc4c(-c5c(-c6ccccc6)ccc6ccccc56)cccc34)nc(-c3cccc4oc5ccccc5c34)n2)cc1 ; c1ccc(-c2nc(-c3cccc4c(-c5cccc6cccc(-c7ccccc7)c56)cccc34)nc(-c3cccc4oc5ccccc5c34)n2)cc1]

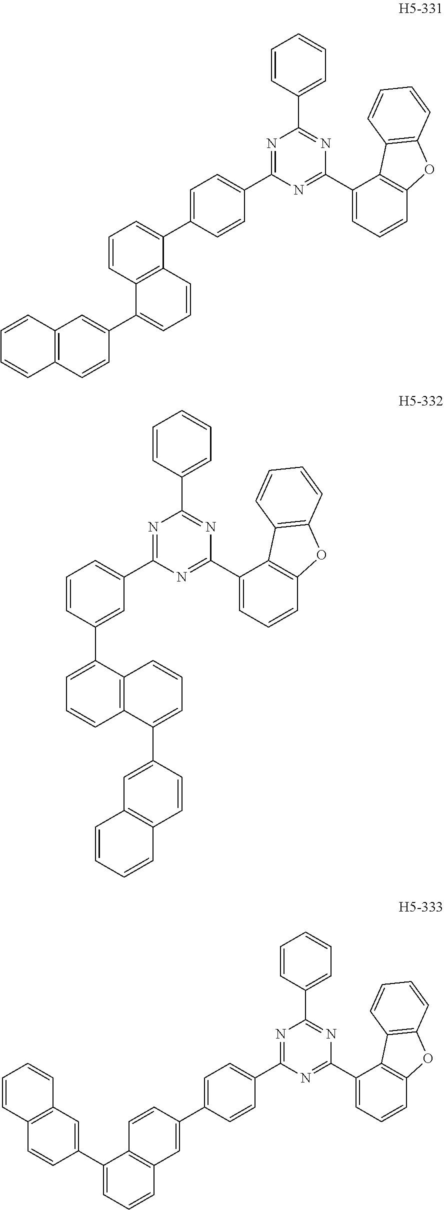 [SMILES: c1ccc(-c2nc(-c3ccc(-c4cccc5c(-c6ccc7ccccc7c6)cccc45)cc3)nc(-c3cccc4oc5ccccc5c34)n2)cc1 ; c1ccc(-c2nc(-c3cccc(-c4cccc5c(-c6ccc7ccccc7c6)cccc45)c3)nc(-c3cccc4oc5ccccc5c34)n2)cc1 ; c1ccc(-c2nc(-c3ccc(-c4ccc5c(-c6ccc7ccccc7c6)cccc5c4)cc3)nc(-c3cccc4oc5ccccc5c34)n2)cc1]

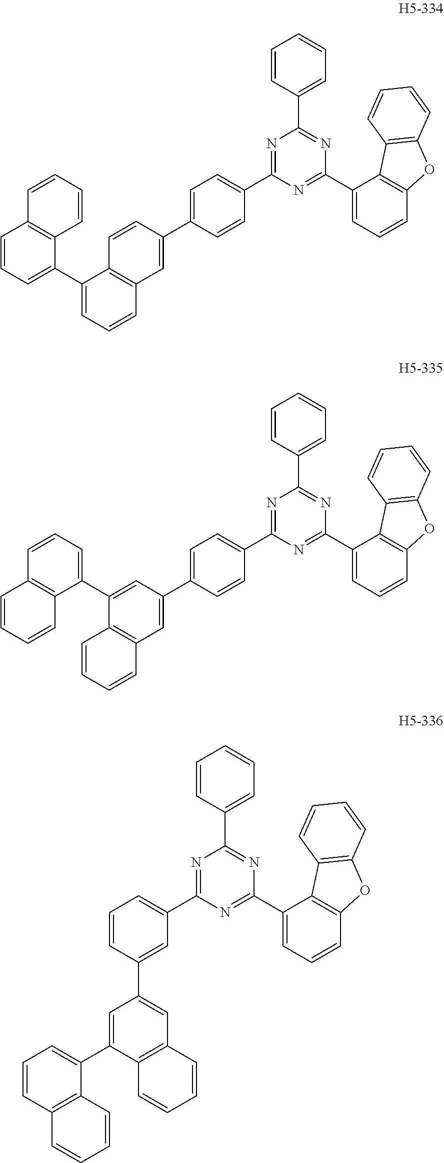 [SMILES: c1ccc(-c2nc(-c3ccc(-c4ccc5c(-c6cccc7ccccc67)cccc5c4)cc3)nc(-c3cccc4oc5ccccc5c34)n2)cc1 ; c1ccc(-c2nc(-c3ccc(-c4cc(-c5cccc6ccccc56)c5ccccc5c4)cc3)nc(-c3cccc4oc5ccccc5c34)n2)cc1 ; c1ccc(-c2nc(-c3cccc(-c4cc(-c5cccc6ccccc56)c5ccccc5c4)c3)nc(-c3cccc4oc5ccccc5c34)n2)cc1]

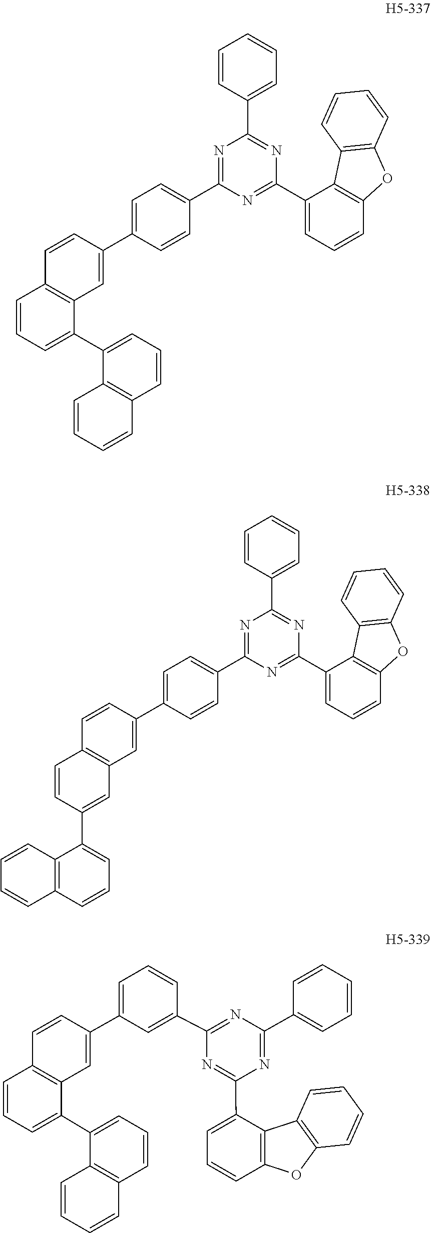 [SMILES: c1ccc(-c2nc(-c3ccc(-c4ccc5cccc(-c6cccc7ccccc67)c5c4)cc3)nc(-c3cccc4oc5ccccc5c34)n2)cc1 ; c1ccc(-c2nc(-c3ccc(-c4ccc5ccc(-c6cccc7ccccc67)cc5c4)cc3)nc(-c3cccc4oc5ccccc5c34)n2)cc1 ; c1ccc(-c2nc(-c3cccc(-c4ccc5cccc(-c6cccc7ccccc67)c5c4)c3)nc(-c3cccc4oc5ccccc5c34)n2)cc1]

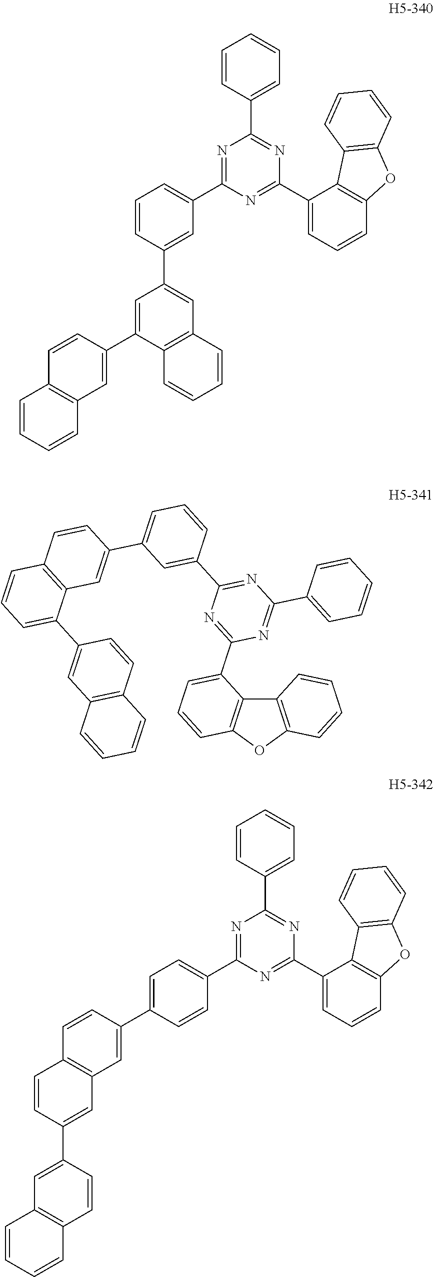 [SMILES: c1ccc(-c2nc(-c3cccc(-c4cc(-c5ccc6ccccc6c5)c5ccccc5c4)c3)nc(-c3cccc4oc5ccccc5c34)n2)cc1 ; c1ccc(-c2nc(-c3cccc(-c4ccc5cccc(-c6ccc7ccccc7c6)c5c4)c3)nc(-c3cccc4oc5ccccc5c34)n2)cc1 ; c1ccc(-c2nc(-c3ccc(-c4ccc5ccc(-c6ccc7ccccc7c6)cc5c4)cc3)nc(-c3cccc4oc5ccccc5c34)n2)cc1]

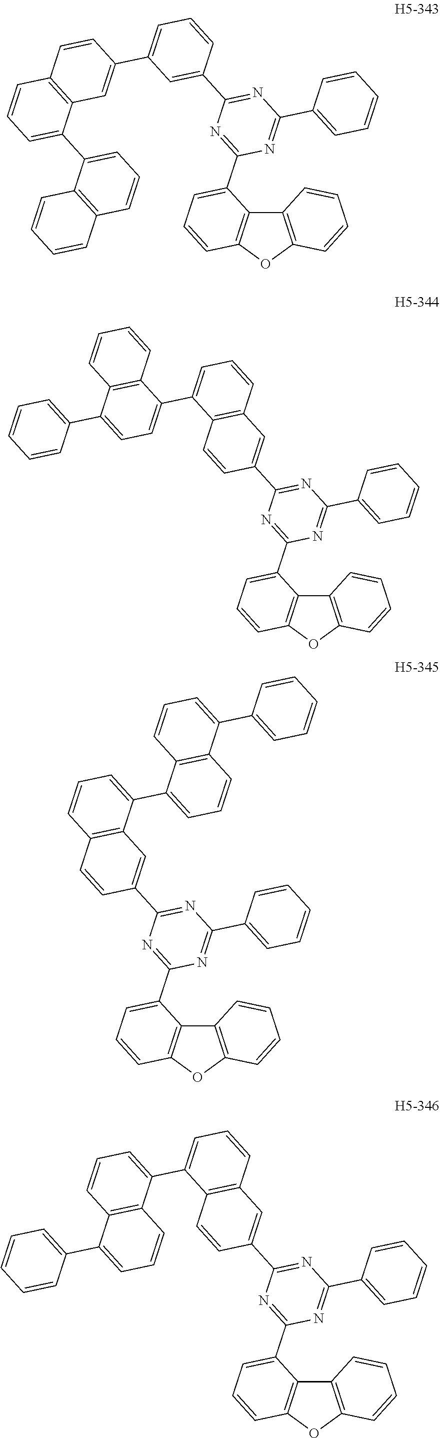

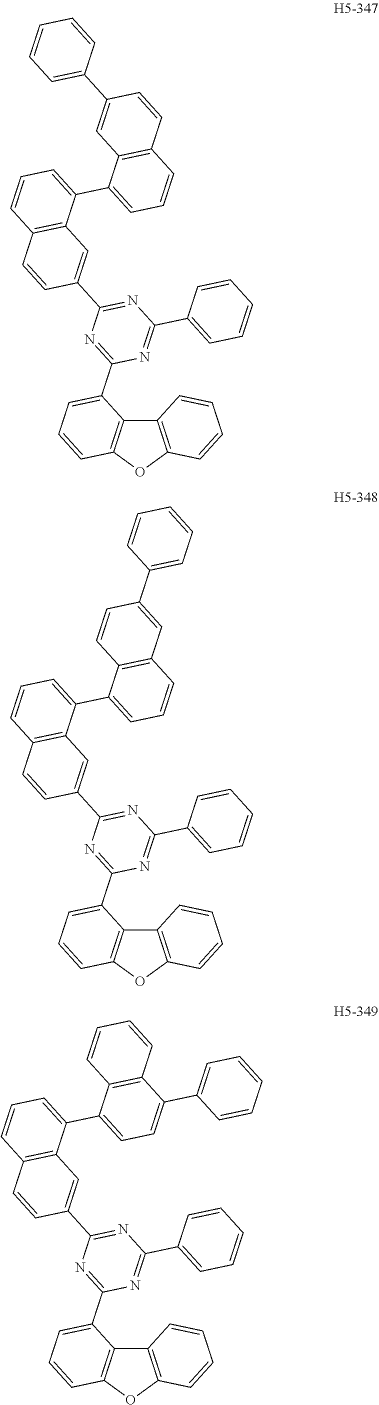

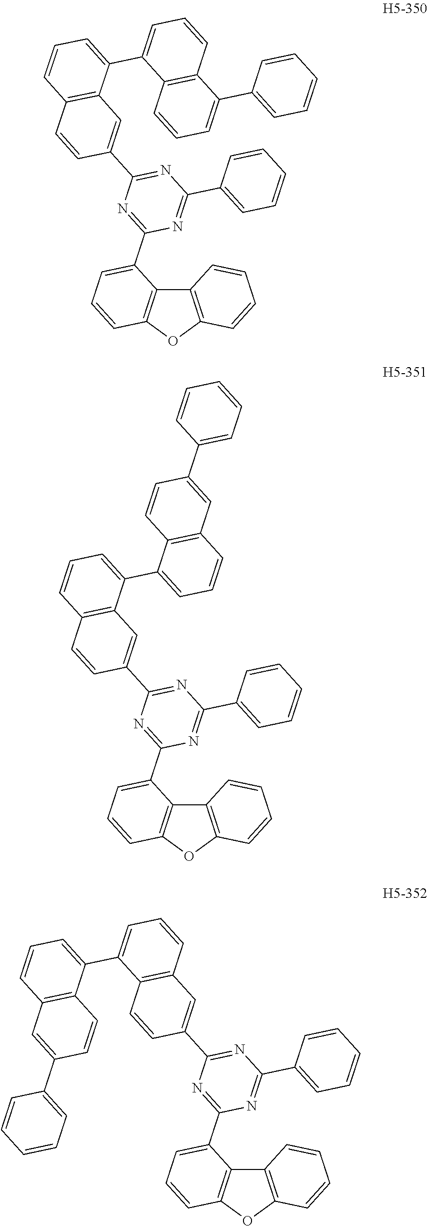 [SMILES: c1ccc(-c2nc(-c3ccc4cccc(-c5cccc6c(-c7ccccc7)cccc56)c4c3)nc(-c3cccc4oc5ccccc5c34)n2)cc1 ; c1ccc(-c2ccc3c(-c4cccc5ccc(-c6nc(-c7ccccc7)nc(-c7cccc8oc9ccccc9c78)n6)cc45)cccc3c2)cc1 ; c1ccc(-c2ccc3c(-c4cccc5cc(-c6nc(-c7ccccc7)nc(-c7cccc8oc9ccccc9c78)n6)ccc45)cccc3c2)cc1]

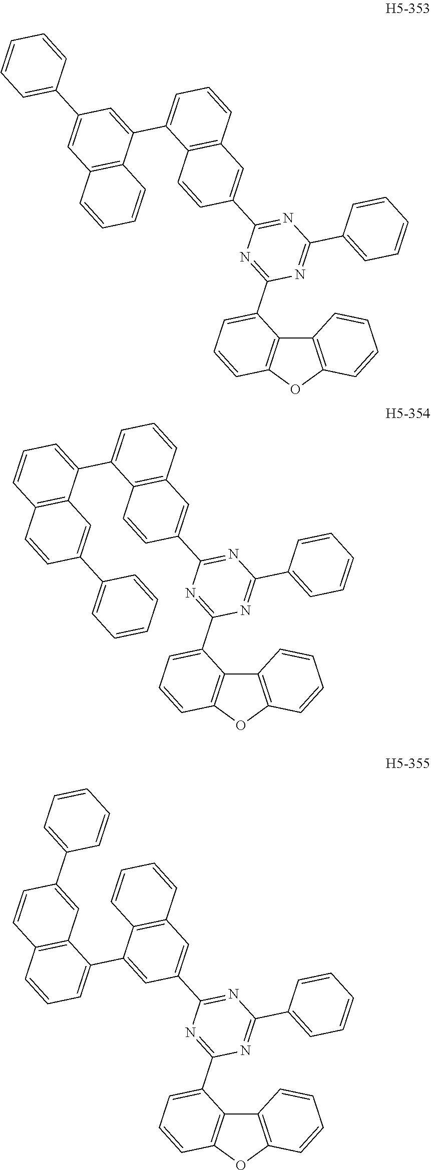 [SMILES: c1ccc(-c2cc(-c3cccc4cc(-c5nc(-c6ccccc6)nc(-c6cccc7oc8ccccc8c67)n5)ccc34)c3ccccc3c2)cc1 ; c1ccc(-c2ccc3cccc(-c4cccc5cc(-c6nc(-c7ccccc7)nc(-c7cccc8oc9ccccc9c78)n6)ccc45)c3c2)cc1 ; c1ccc(-c2ccc3cccc(-c4cc(-c5nc(-c6ccccc6)nc(-c6cccc7oc8ccccc8c67)n5)cc5ccccc45)c3c2)cc1]

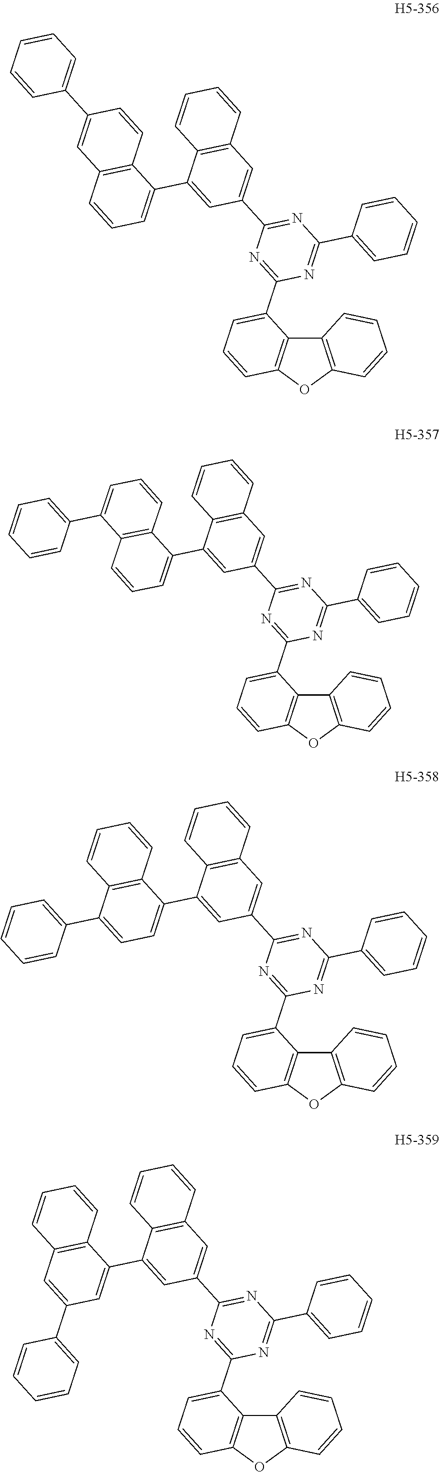 [SMILES: c1ccc(-c2ccc3c(-c4cc(-c5nc(-c6ccccc6)nc(-c6cccc7oc8ccccc8c67)n5)cc5ccccc45)cccc3c2)cc1 ; c1ccc(-c2nc(-c3cc(-c4cccc5c(-c6ccccc6)cccc45)c4ccccc4c3)nc(-c3cccc4oc5ccccc5c34)n2)cc1 ; c1ccc(-c2nc(-c3cc(-c4ccc(-c5ccccc5)c5ccccc45)c4ccccc4c3)nc(-c3cccc4oc5ccccc5c34)n2)cc1 ; c1ccc(-c2cc(-c3cc(-c4nc(-c5ccccc5)nc(-c5cccc6oc7ccccc7c56)n4)cc4ccccc34)c3ccccc3c2)cc1]

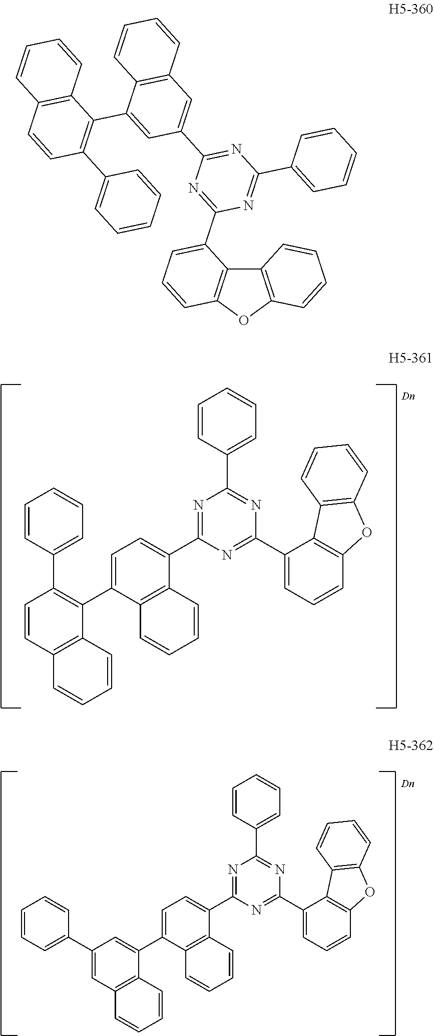

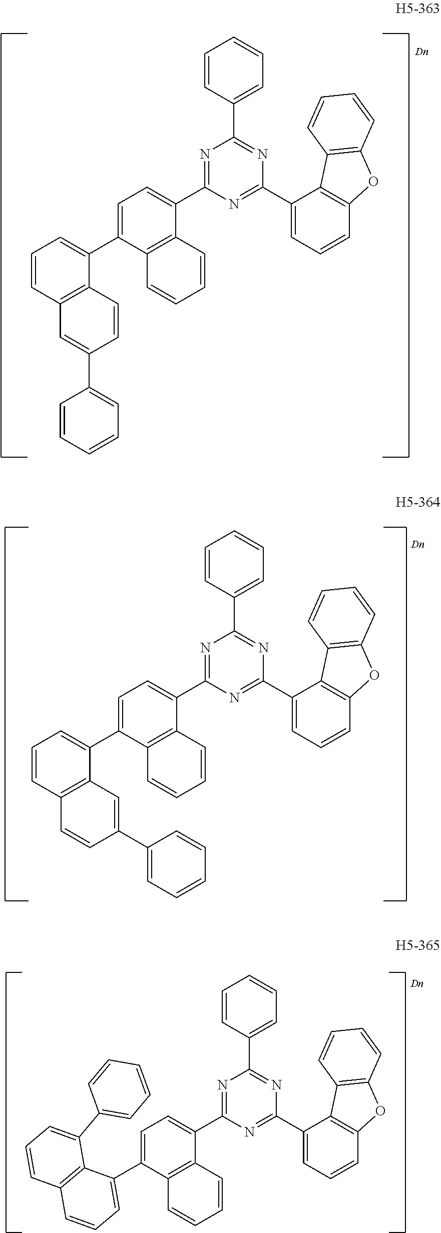

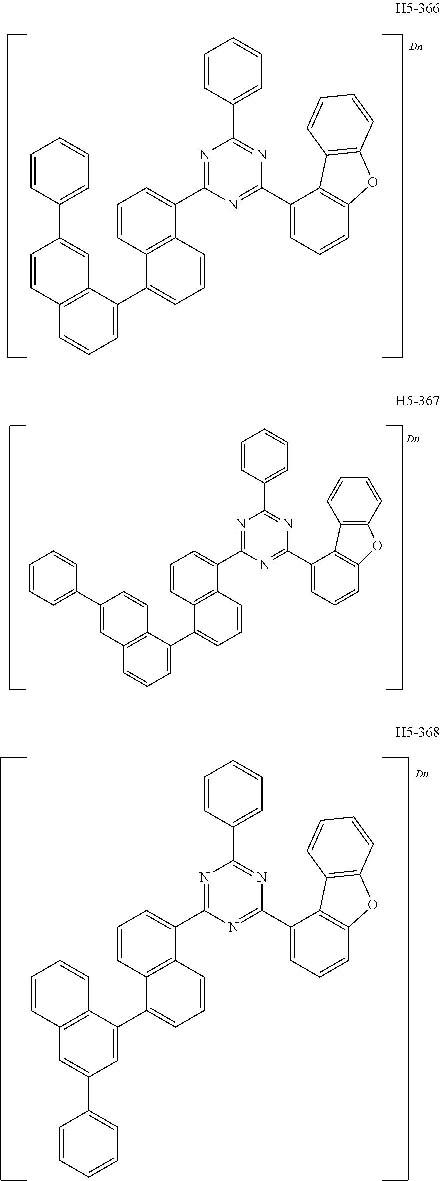

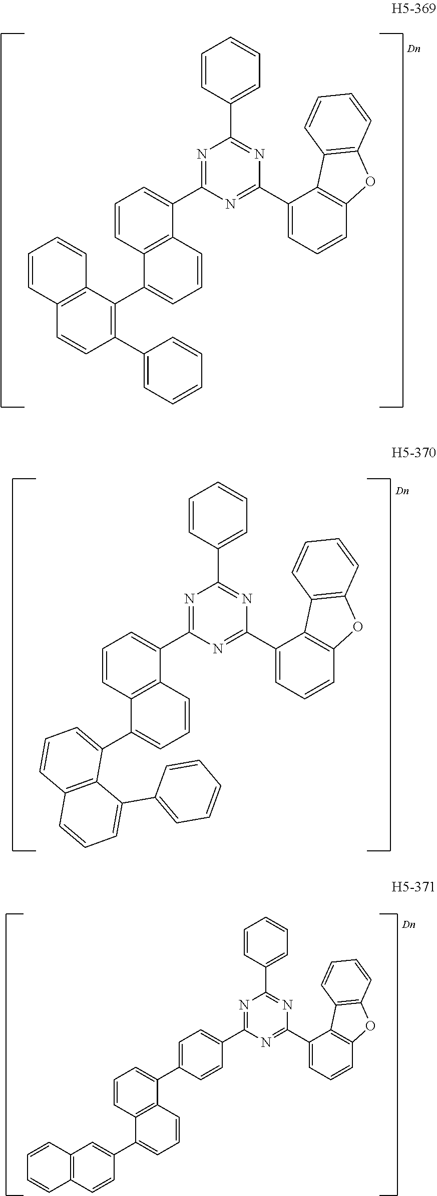

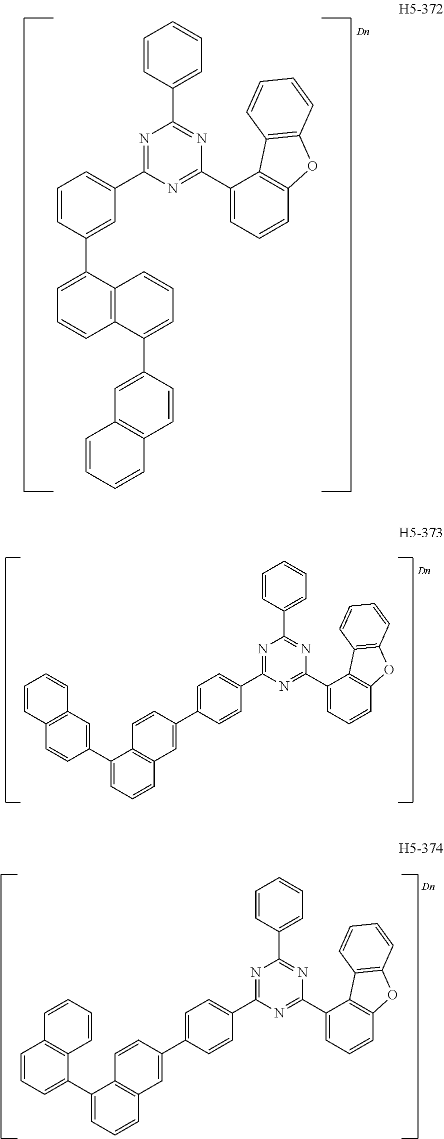

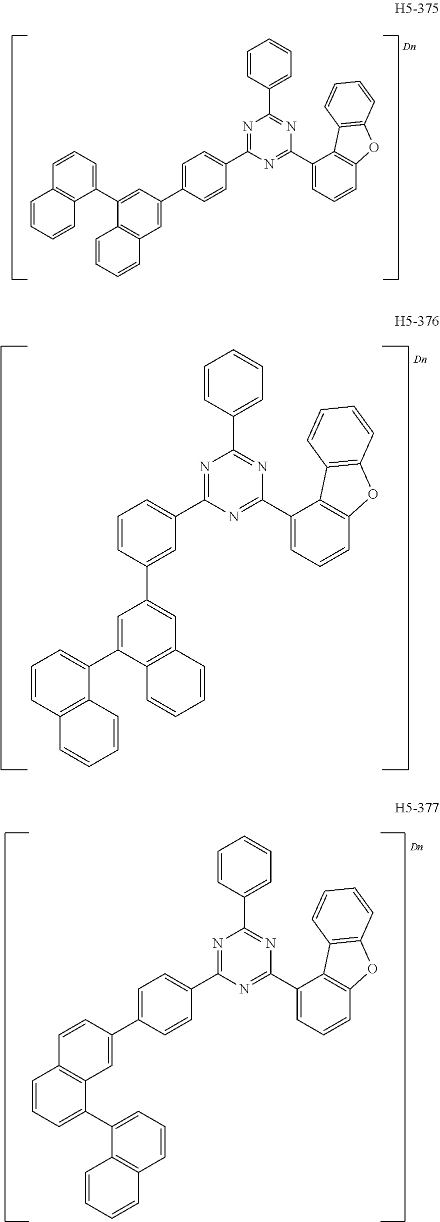

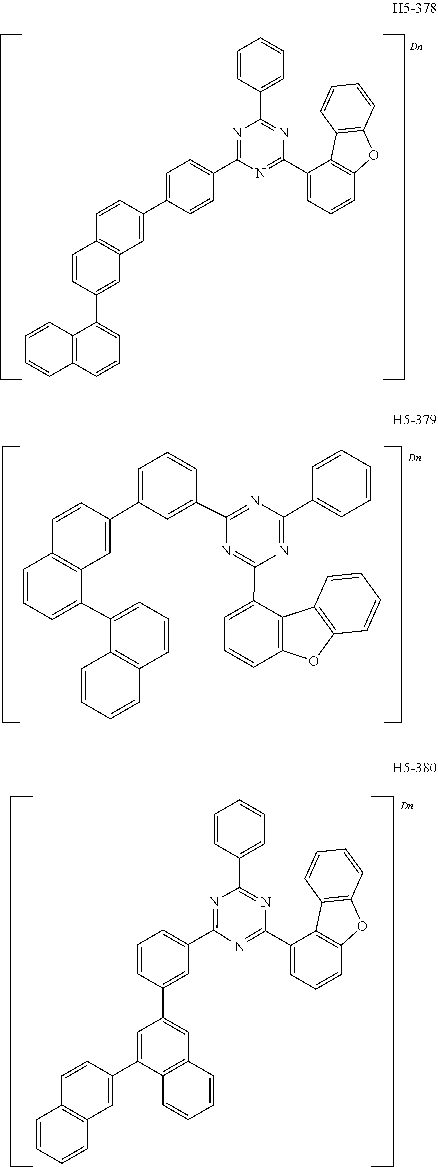

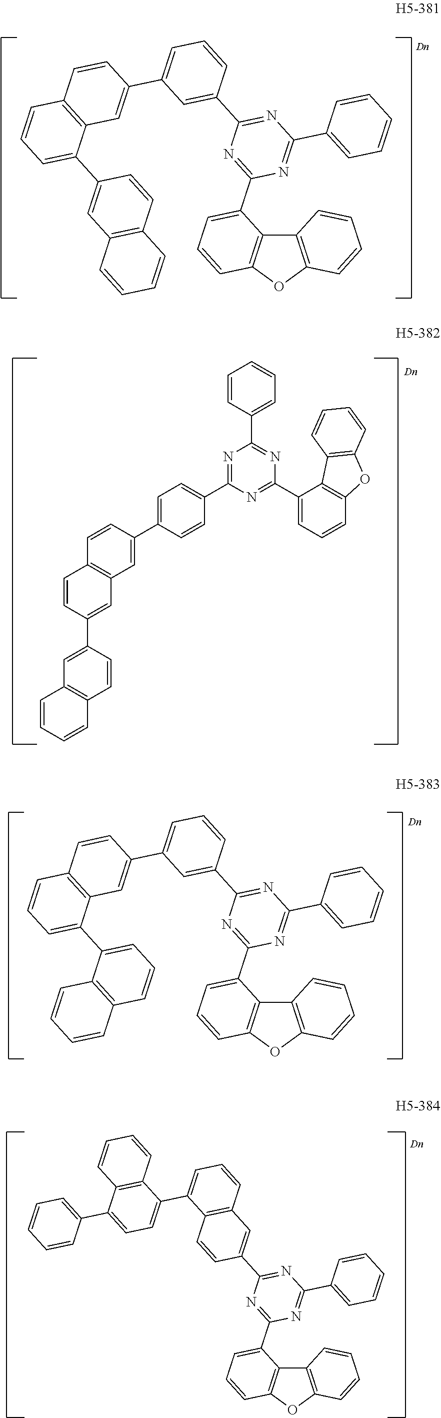

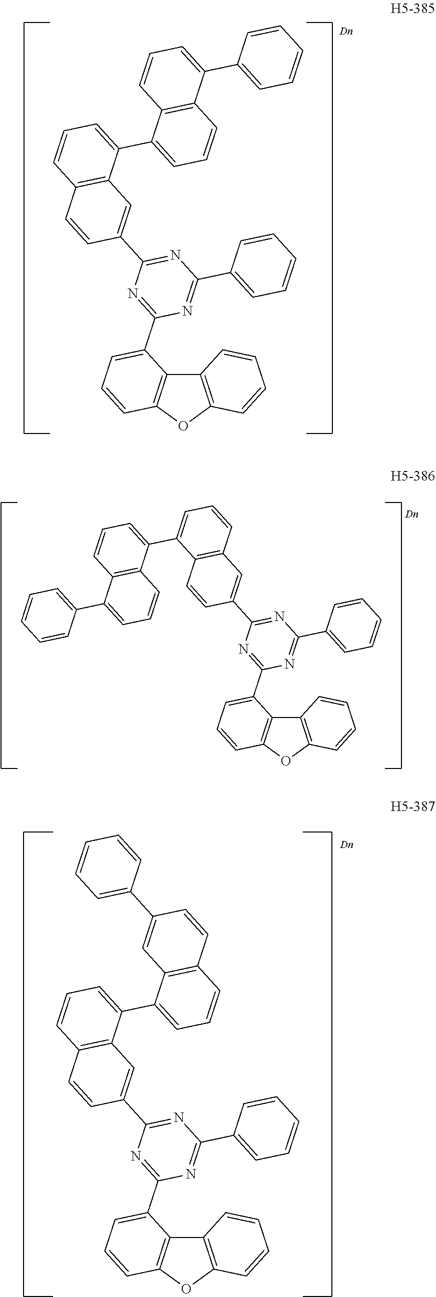

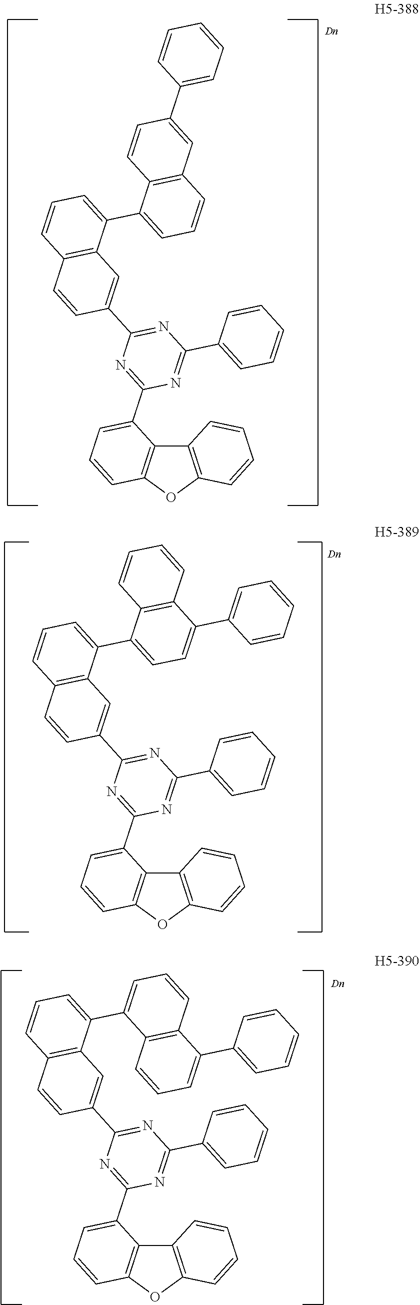

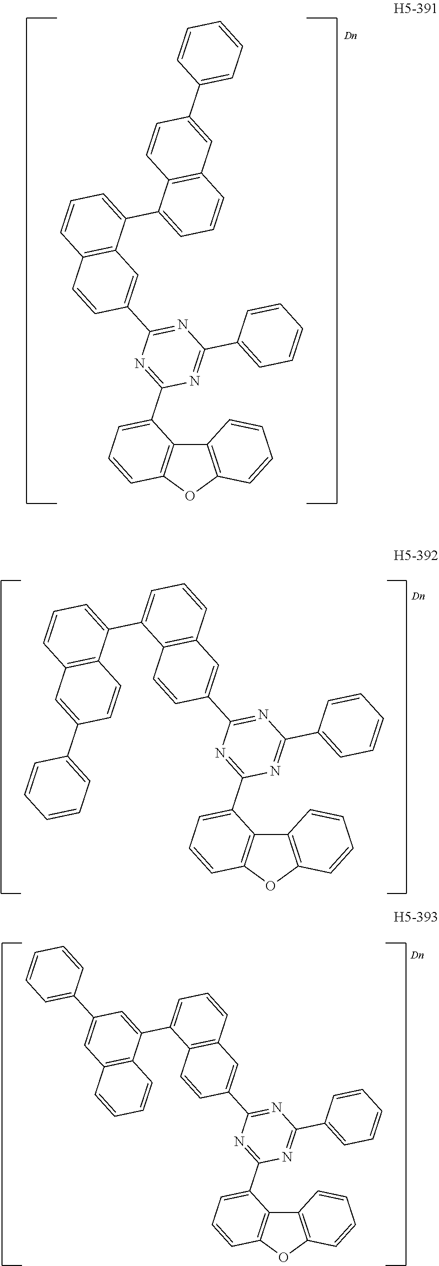

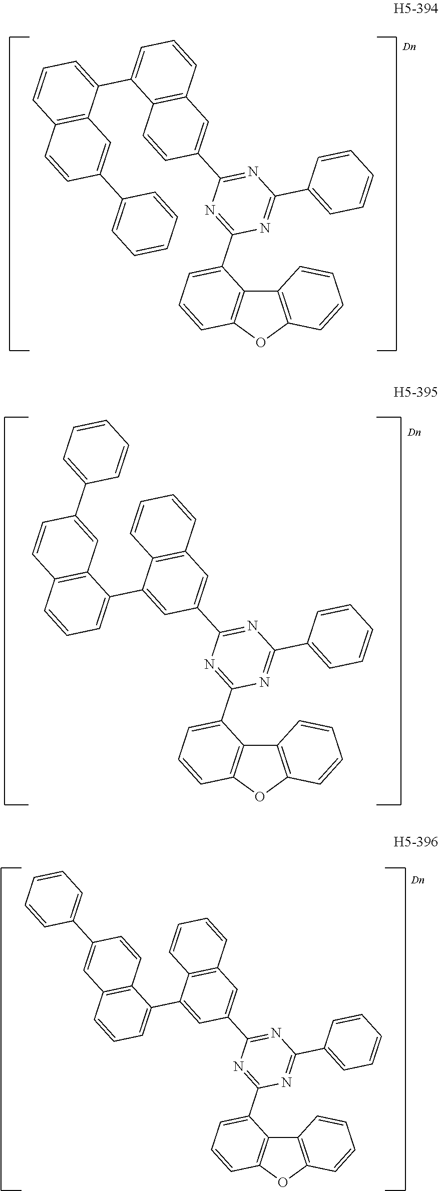

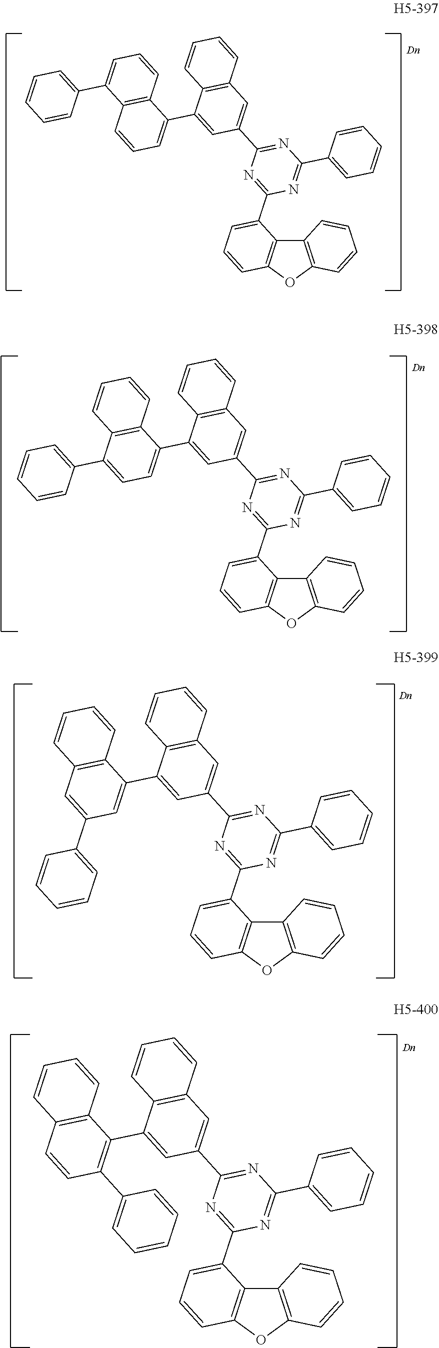

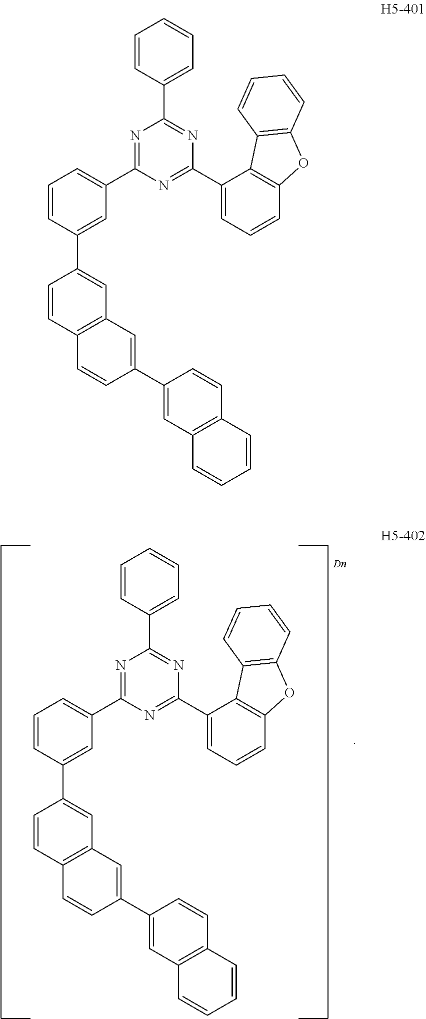

In the compounds above, Dₙ means that n number of hydrogens is replaced with deuterium, wherein n is an integer of 1 or more, and the upper limit of n is determined according to the number of hydrogens that may be substituted for each compound.

Hereinafter, an organic electroluminescent device comprising the aforementioned compound represented by Formulas 1 to 3, 1-1, and 3′ will be described.

An organic electroluminescent device according to one embodiment includes a first electrode and a second electrode facing each other on a substrate, at least one light-emitting layer positioned between the first electrode and the second electrode, and at least one electron blocking layer positioned between the first electrode and the light-emitting layer.

According to one embodiment, the first electrode may be an anode and the second electrode may be a cathode. Wherein, the first electrode and the second electrode may each be formed as a transmissive conductive material, a transflective conductive material, or a reflective conductive material. The organic electroluminescent device may be a top emission type, a bottom emission type, or a both-sides emission type depending on the kinds of the material forming the first electrode and the second electrode.

The electron blocking layer is located between the first electrode and the light-emitting layer, preferably between the hole transport layer and the light-emitting layer, and more preferably, the electron blocking layer is in contact with the light-emitting layer. The electron blocking layer according to one example can improve the efficiency of the organic electroluminescent device by suppressing electrons injected from the cathode from being transferred toward the anode without being recombined in the light-emitting layer. In addition, it can prevent emission leakage by blocking the overflow of electrons from the light-emitting layer and confining excitons within the light-emitting layer.

According to one embodiment, the electron blocking layer includes a compound represented by Formula 1, and the light-emitting layer can include a compound represented by Formula 2 as a first host compound and a compound represented by Formula 3 as a second host compound, respectively. Wherein, the weight ratio of the first host compound to the second host compound may be included in the light-emitting layer in a range of about 1:99 to about 99:1, preferably about 10:90 to about 90:10, more preferably about 30:70 to about 70:30, more preferably about 40:60 to about 60:40, and even more preferably about 50:50.

According to one embodiment, the organic electroluminescent device comprises at least one compound among Compounds H1-1 to H1-161 as an electron blocking layer material, and at least one compound among Compounds H3-1 to H3-743 and H4-1 to H4-484 and at least one compound among Compounds H5-1 to H5-402 as a host material of the light-emitting layer, wherein these host materials may be included in the same light-emitting layer, and may be included in different light-emitting layers, respectively.

According to one embodiment, the present invention provides an organic electroluminescent device comprising an organic electroluminescent compound represented by Formula 1-1.

According to one embodiment, the present invention provides an organic electroluminescent device comprising an organic electroluminescent compound represented by Formula 3′.

An organic electroluminescent device according to one embodiment includes a hole injection layer, a hole transport layer, a hole auxiliary layer, an electron transport layer, and an electron injection layer, as an organic layer disposed between a first electrode and a second electrode, in addition to the electron blocking layer and the light-emitting layer, and may further include one or more layers selected from a light-emitting auxiliary layer, an interlayer, a hole blocking layer, and an electron buffer layer. The organic layer may further include an amine-based compound and/or an azine-based compound in addition to the light-emitting material of the present disclosure. Specifically, the hole injection layer, the hole transport layer, the hole auxiliary layer, the light-emitting layer, the light-emitting auxiliary layer, or the electron blocking layer may include an amine-based compound, for example, an arylamine-based compound, a styrylarylamine-based compound, or the like, as a hole injection material, a hole transport material, a hole auxiliary material, a light-emitting material, a light-emitting auxiliary material, and an electron blocking material. In addition, the electron transport layer, electron injection layer, electron buffer layer and hole blocking layer may include an azine-based compound as an electron transport material, an electron injection material, an electron buffer material and a hole blocking material. Also, the organic layer may further comprise at least one metal selected from the group consisting of metals of Group 1, metals of Group 2, transition metals of the 4th period, transition metals of the 5th period, lanthanides, and organic metals of the d-transition elements of the Periodic Table, or at least one complex compound comprising such a metal.

A hole injection layer, a hole transport layer, an electron blocking layer, or a combination thereof can be used between the anode and the light-emitting layer. The hole injection layer may be multi-layers in order to lower the hole injection barrier (or hole injection voltage) from the anode to the hole transport layer or the electron blocking layer, wherein each of the multi-layers may use two compounds simultaneously. Also, the hole injection layer may be doped as a p-dopant.

The hole transport layer or the electron blocking layer according to one embodiment may be multi-layers, wherein each layer may use a plurality of compounds.

The hole auxiliary layer is located between the hole transport layer and the electron blocking layer (or the light-emitting layer), and can exhibit the effect of facilitating or blocking the hole transport speed (or injection speed), thereby controlling the charge balance to effectively lower the driving voltage of the organic electroluminescent device. When the organic electroluminescent device includes two or more hole transport layers, the additionally included hole transport layer can also be used as the hole auxiliary layer or the electron blocking layer.

An electron buffer layer, a hole-blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole-blocking layer may be placed between the electron transport layer (or electron injection layer) and the light-emitting layer, and blocks the arrival of holes to the cathode, thereby improving the probability of recombination of electrons and holes in the light-emitting layer. The hole-blocking layer or the electron transport layer may also be multi-layers, wherein each layer may use a plurality of compounds. Also, the electron injection layer may be doped as an n-dopant.

The organic electroluminescent device of the present disclosure may further include the light-emitting auxiliary layer placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. The light-emitting auxiliary layer, the hole auxiliary layer, or the electron blocking layer may have an effect of improving the efficiency and/or the lifespan of the organic electroluminescent device.

The organic electroluminescent material according to one embodiment may be used as light-emitting materials for a white organic light-emitting device. The white organic light-emitting device has various suggested structures such as a parallel side-by-side arrangement method, a stacking arrangement method, or a CCM (color conversion material) method, etc., depending on the arrangement of R (red), G (green), YG (yellowish green), or B (blue) light-emitting units. In addition, the compound or the organic electroluminescent material according to one embodiment may also be applied to the organic electroluminescent device comprising a QD (quantum dot).

In the organic electroluminescent device of the present disclosure, preferably at least one layer (hereinafter, “a surface layer”) selected from a chalcogenide layer, a halogenated metal layer, and a metal oxide layer may be placed on an inner surface(s) of one or both of a pair of electrodes. Specifically, a chalcogenide (including oxides) layer of silicon and aluminum is preferably placed on an anode surface of an electroluminescent medium layer, and a halogenated metal layer or a metal oxide layer is preferably placed on a cathode surface of an electroluminescent medium layer. The operation stability for the organic electroluminescent device may be obtained by the surface layer. Preferably, the chalcogenide includes SiOₓ(1≤X≤2), AlOₓ(1≤X≤1.5), SiON, SiAlON, etc.; the halogenated metal includes LiF, MgF₂, CaF₂, a rare earth metal fluoride, etc.; and the metal oxide includes Cs₂O, Li₂O, MgO, SrO, BaO, CaO, etc.

The organic electroluminescent device according to one embodiment of the present disclosure may be an organic electroluminescent device having a tandem structure. In the case of a tandem organic electroluminescent device according to one embodiment, a single light-emitting unit (light-emitting unit) may be formed in a structure in which two or more units are connected by a charge generation layer. The organic electroluminescent device may include a plurality of two or more light-emitting units, for example, a plurality of three or more light-emitting units, having first and second electrodes opposed to each other on a substrate and a light-emitting layer that is stacked between the first and second electrodes and emits light in a specific wavelength range. According to one embodiment, the organic electroluminescent device may include a plurality of light-emitting units, and each of the light-emitting units may include a hole transport zone, a light-emitting layer, and an electron transport zone, and the hole transport band may include a hole injection layer and a hole transport layer, and the electron transport zone may include an electron transport layer and an electron injection layer. According to one embodiment, three or more light-emitting layers may be included in the light-emitting unit. A plurality of light-emitting units may emit the same color or different colors. Additionally, one light-emitting unit may include one or more light-emitting layers, and the plurality of light-emitting layers may be light-emitting layers of the same or different colors. This may include one or more charge generation layers located between each light-emitting unit. The charge generation layer refers to the layer in which holes and electrons are generated when voltage is applied. When there are three or more light-emitting units, a charge generation layer may be located between each light-emitting unit. Here, the plurality of charge generation layers may be the same as or different from each other. By disposing the charge generation layer between light-emitting units, current efficiency is increased in each light-emitting unit, and charges can be smoothly distributed. Specifically, the charge generation layer is provided between two adjacent stacks and can serve to drive a tandem organic electroluminescent device using only a pair of anode and cathode without a separate internal electrode located between the stacks.

The charge generation layer may be composed of an n-type charge generation layer and a p-type charge generation layer, and the n-type charge generation layer may be doped with an alkali metal, an alkaline earth metal, or a compound of an alkali metal and an alkaline earth metal. The alkali metal may include one selected from the group consisting of Li, Na, K, Rb, Cs, Fr, Yb, and combinations thereof, and the alkaline earth metal may include one selected from the group consisting of Be, Mg, Ca, Sr, Ba, Ra, and combinations thereof. The p-type charge generation layer may be made of a metal or an organic material doped with a p-type dopant. For example, the metal may be made of one or two or more alloys selected from the group consisting of Al, Cu, Fe, Pb, Zn, Au, Pt, W, In, Mo, Ni, and Ti. Additionally, commonly used materials may be used as the p-type dopant and host materials used in the p-type doped organic material.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation; thus, it becomes easier to inject and transport holes from the mixed region to the electroluminescent medium. Preferably, the oxidative dopant includes various Lewis acids and acceptor compounds, and the reductive dopant includes alkali metals, alkali metal compounds, alkaline earth metals, rare earth metals, and mixtures thereof. Also, a reductive dopant layer may be employed as a charge generating layer to prepare an organic electroluminescent device having two or more light-emitting layers and emitting white light.

An organic electroluminescent device according to one embodiment may further comprise at least one dopant in the light-emitting layer.

The dopant comprised in the organic electroluminescent device of the present disclosure may be at least one phosphorescent or fluorescent dopant, preferably a phosphorescent dopant. The phosphorescent dopant material applied to the organic electroluminescent device of the present disclosure is not particularly limited, but may preferably be a metallated complex compound(s) of a metal atom(s) selected from iridium (Ir), osmium (Os), copper (Cu), and platinum (Pt), more preferably an ortho-metallated complex compound(s) of a metal atom(s) selected from iridium (Ir), osmium (Os), copper (Cu), and platinum (Pt), and even more preferably ortho-metallated iridium complex compound(s).

The dopant comprised in the organic electroluminescent device of the present disclosure may use the compound represented by the following Formula 101, but is not limited thereto.

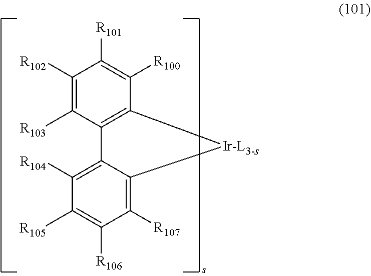

In Formula 101,
L is any one selected from the following structures 1 to 3:

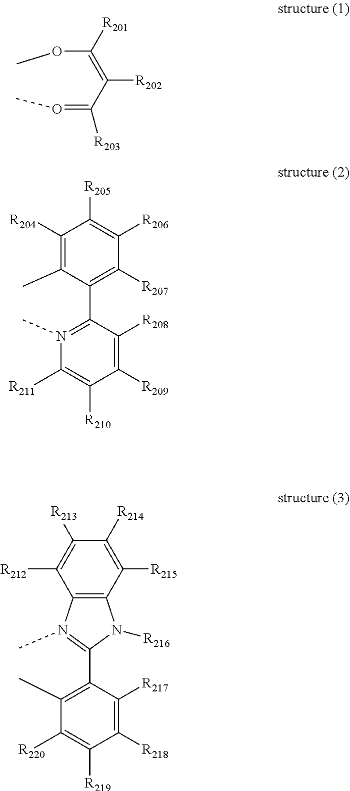

in structures 1 to 3,
R₁₀₀ to R₁₀₃ each independently represent hydrogen, deuterium, a halogen, (C1-C30)alkyl unsubstituted or substituted with deuterium and/or halogen, a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to the adjacent substituents to form a ring(s), for example, to form a ring(s) with a pyridine, e.g., a substituted or unsubstituted quinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline;
R₁₀₄ to R₁₀₇ each independently represent hydrogen, deuterium, a halogen, (C1-C30)alkyl unsubstituted or substituted with deuterium and/or halogen, a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent(s) to form a substituted or unsubstituted ring(s), for example, to form a substituted or unsubstituted ring(s) with a benzene, e.g., a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine;
R₂₀₁ to R₂₂₀ each independently represent hydrogen, deuterium, a halogen, (C1-C30)alkyl unsubstituted or substituted with deuterium and/or halogen, a substituted or unsubstituted (C3-C30)cycloalkyl, or a substituted or unsubstituted (C6-C30)aryl; or may be linked to an adjacent substituent(s) to form a substituted or unsubstituted ring(s); and
s represents an integer of 1 to 3.

Specifically, the specific examples of the dopant compound include the following, but are not limited thereto.

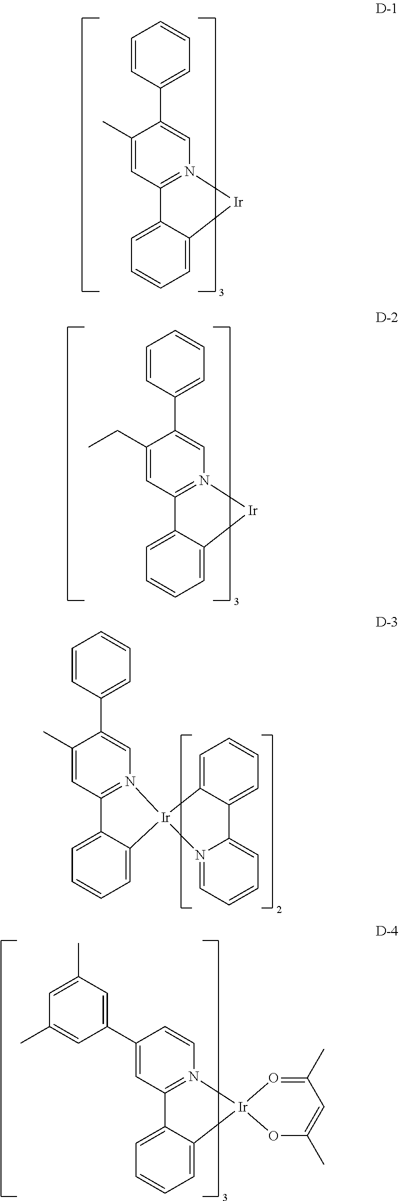

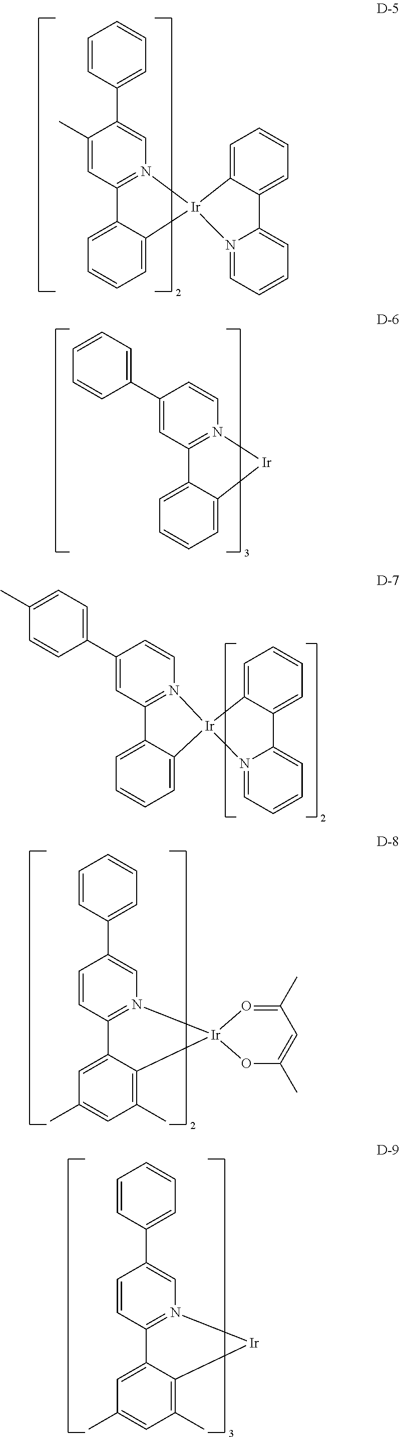

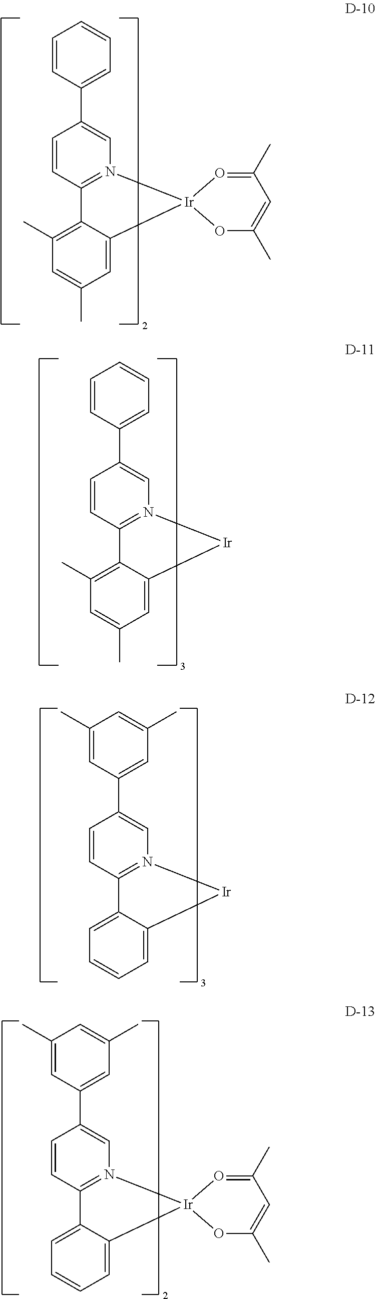

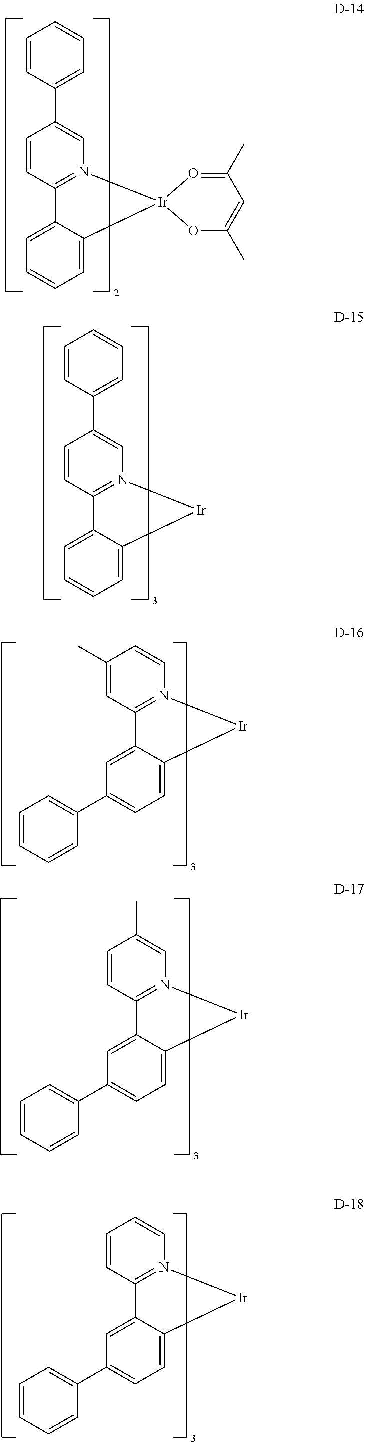

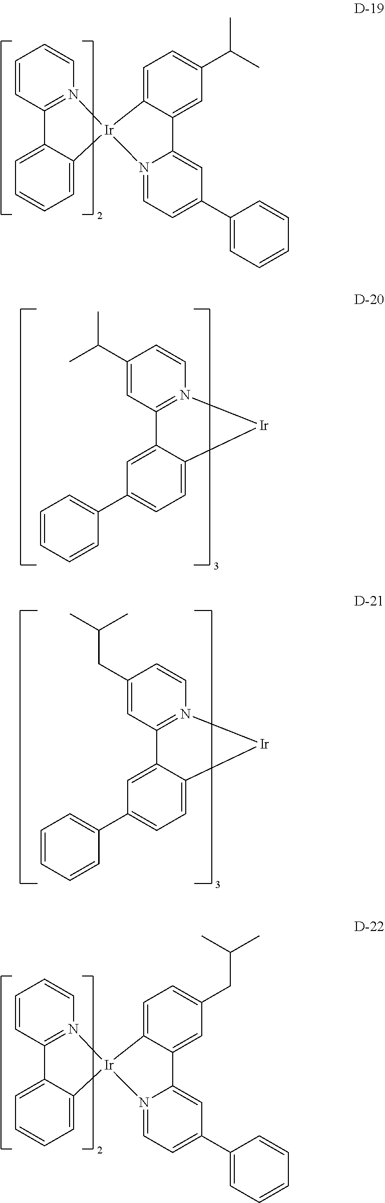

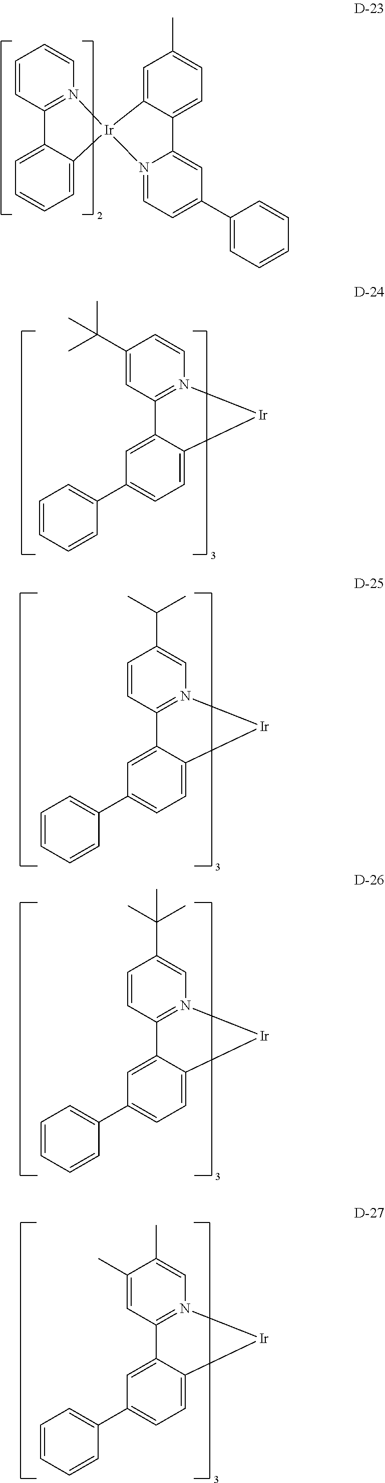

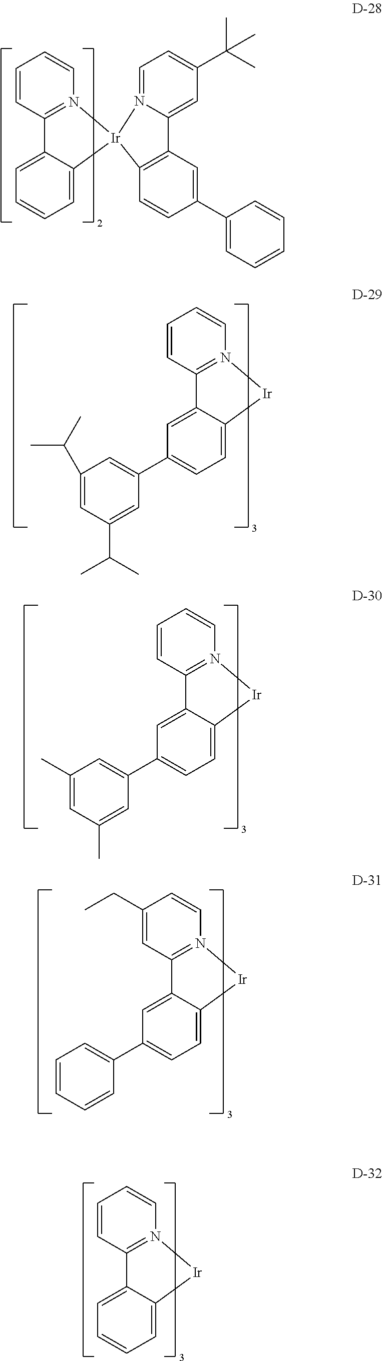

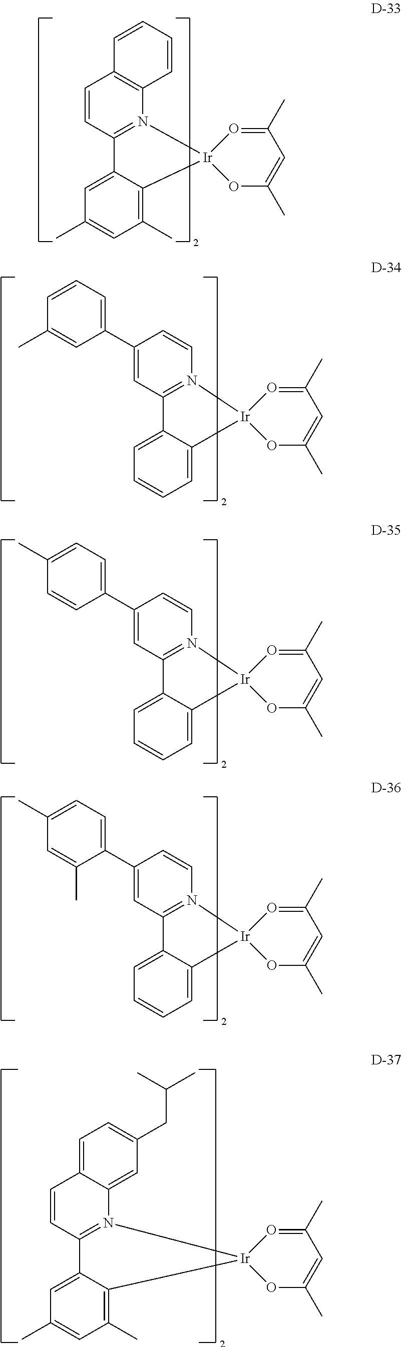

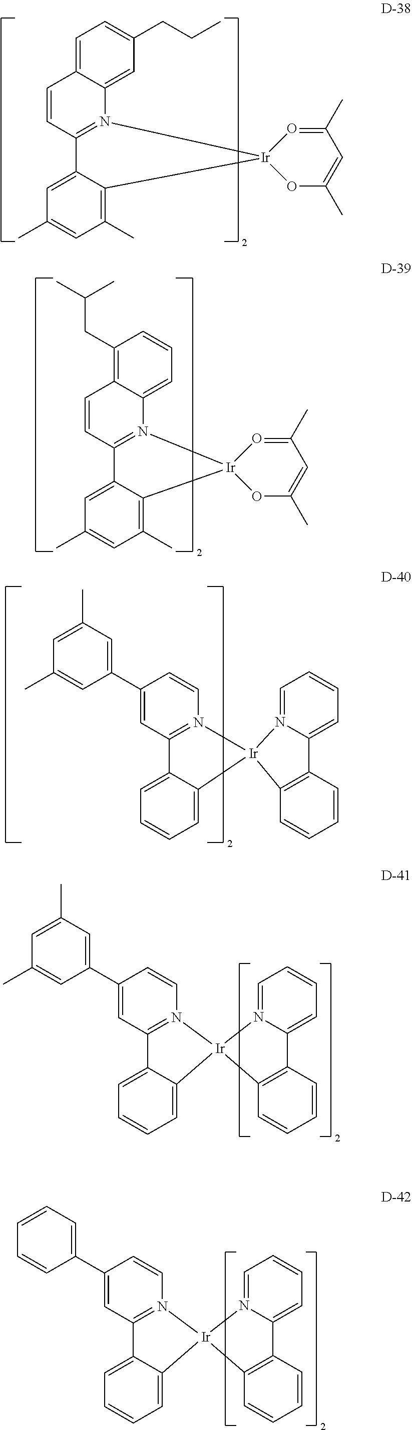

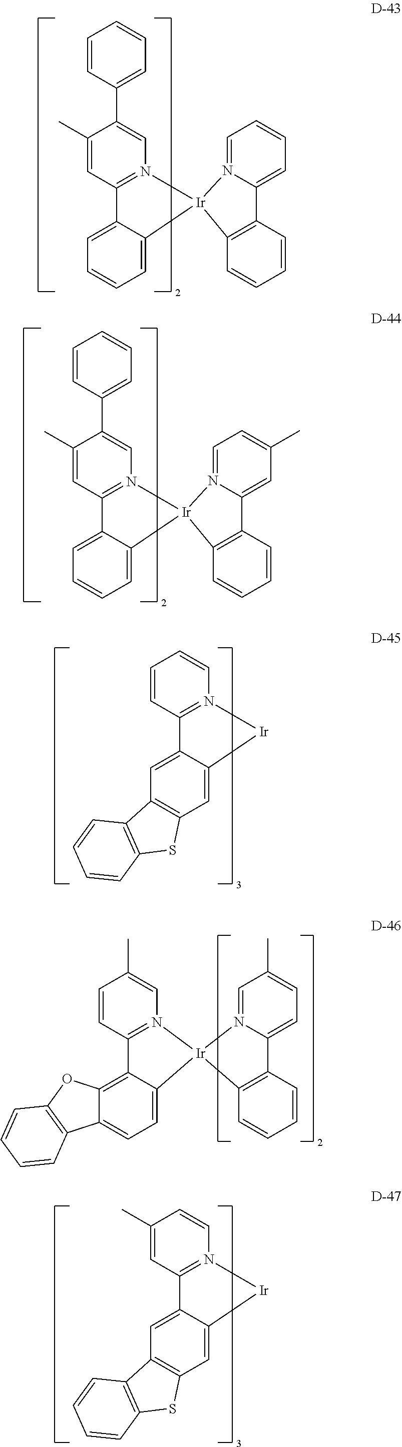

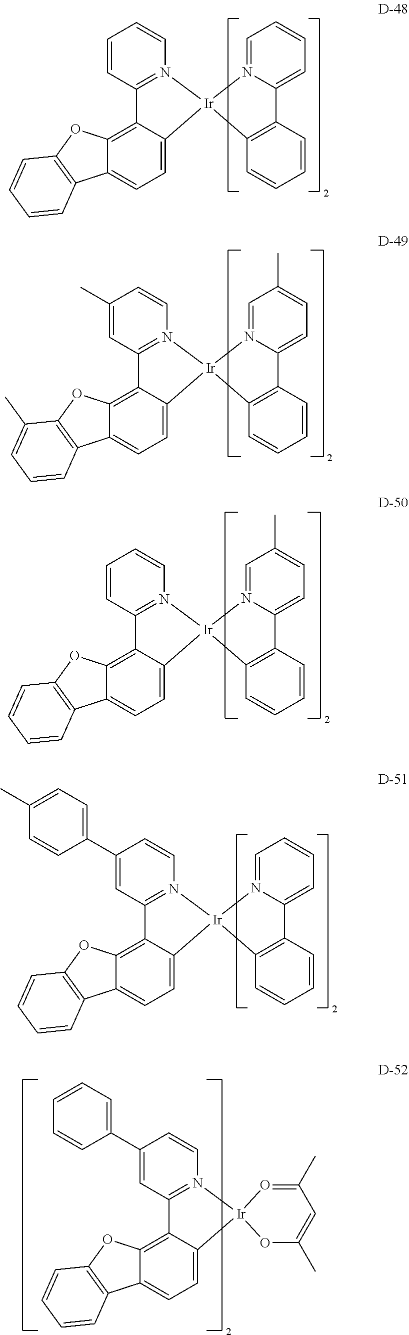

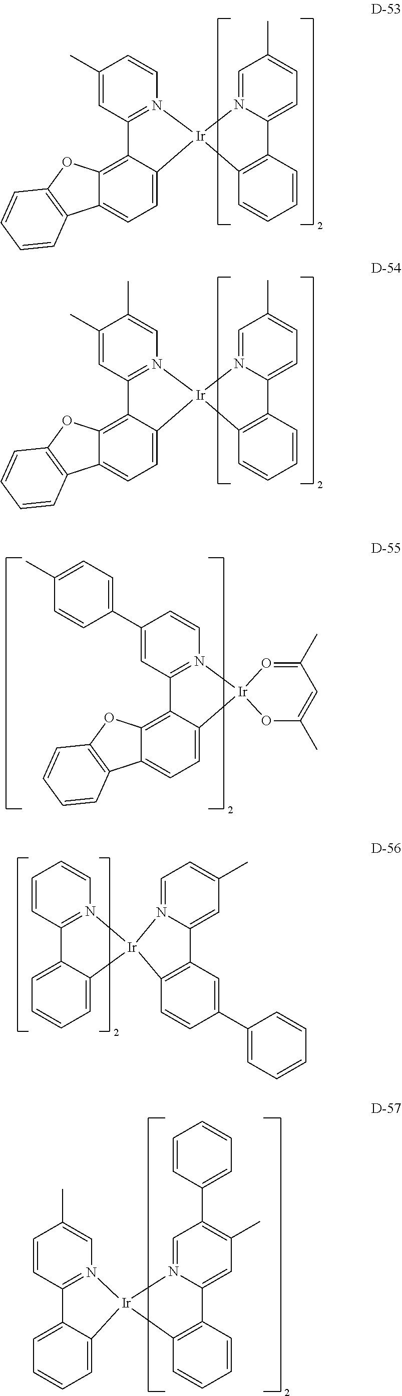

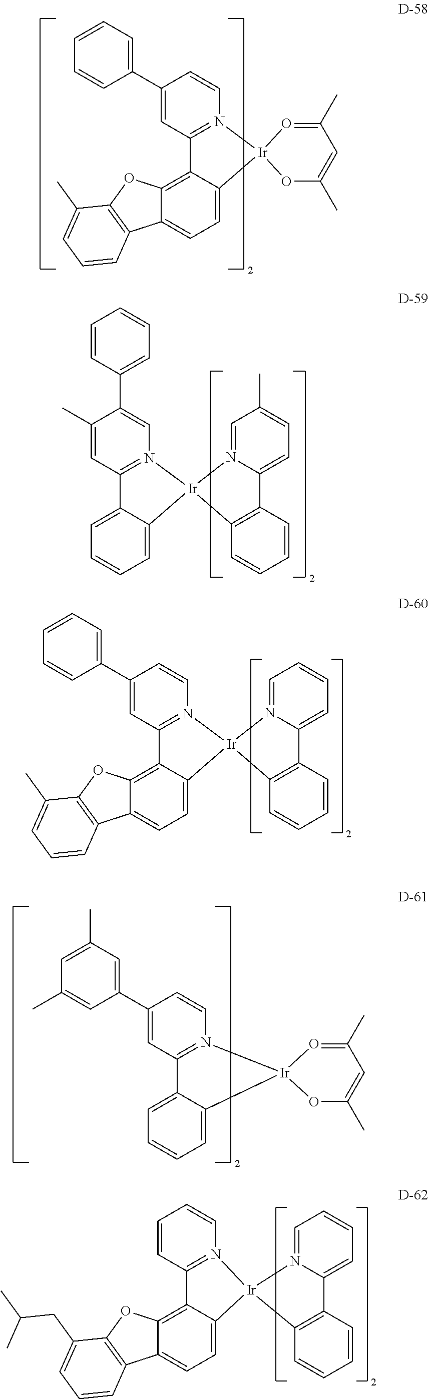

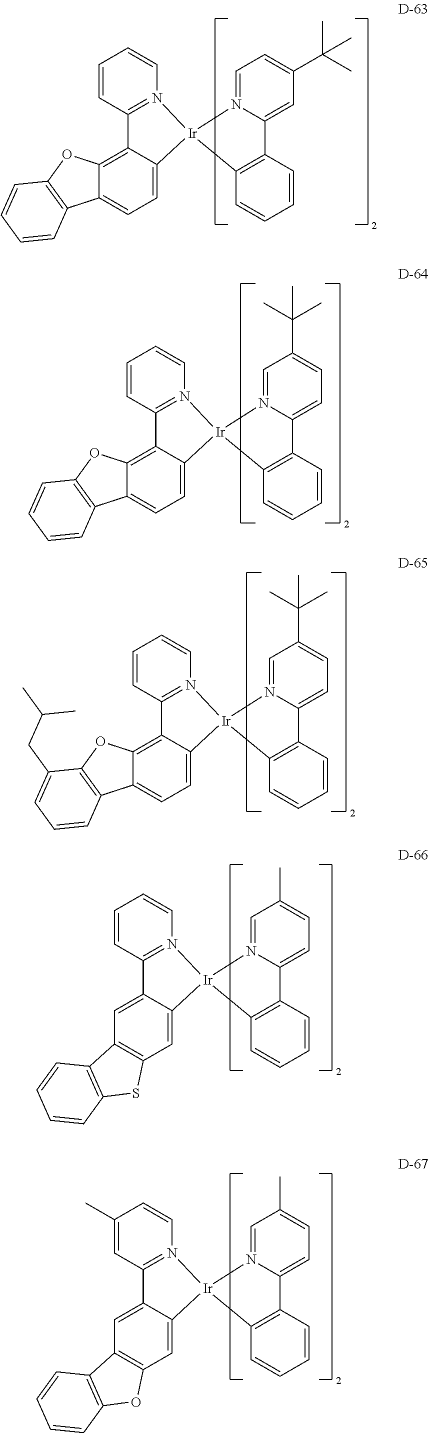

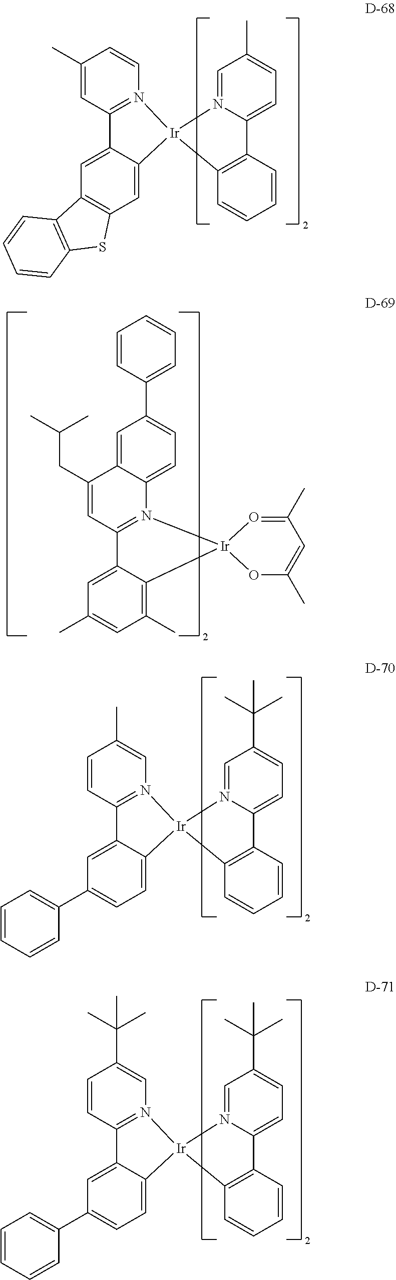

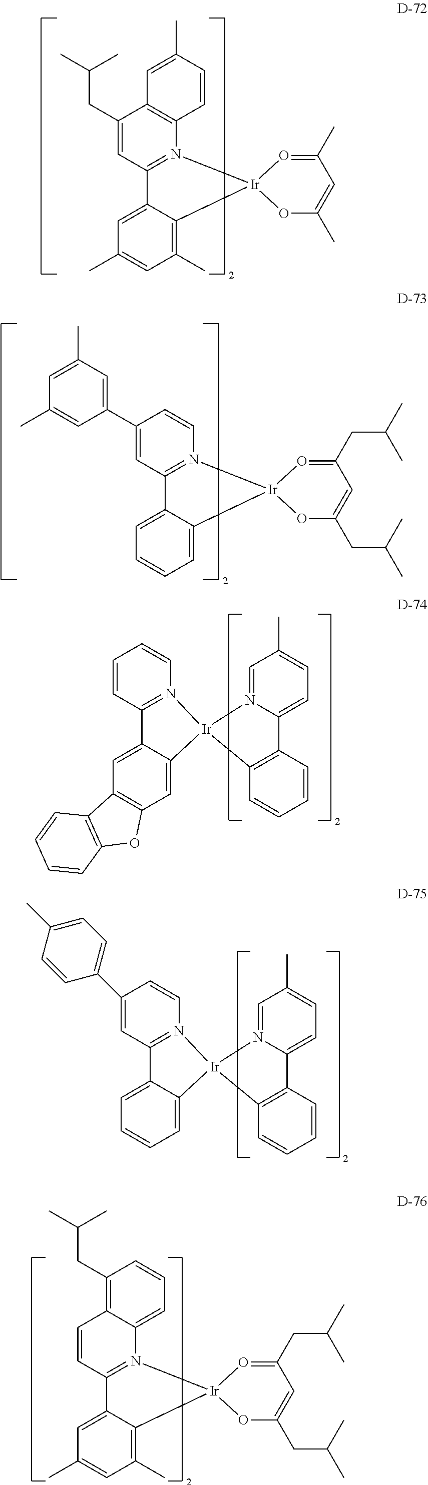

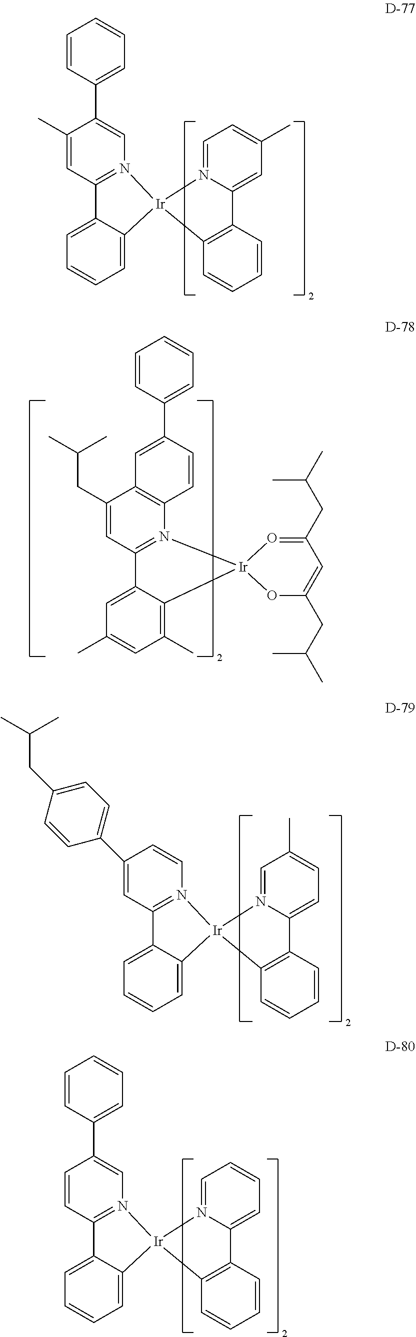

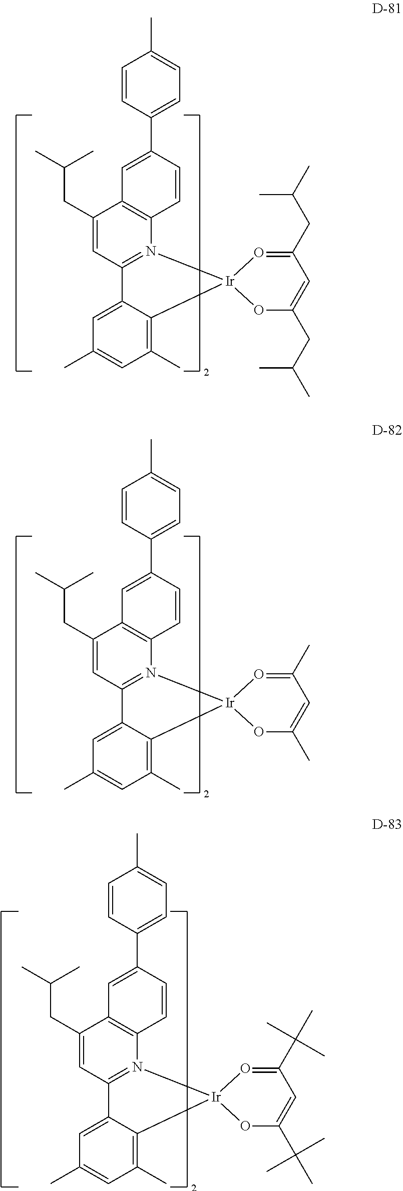

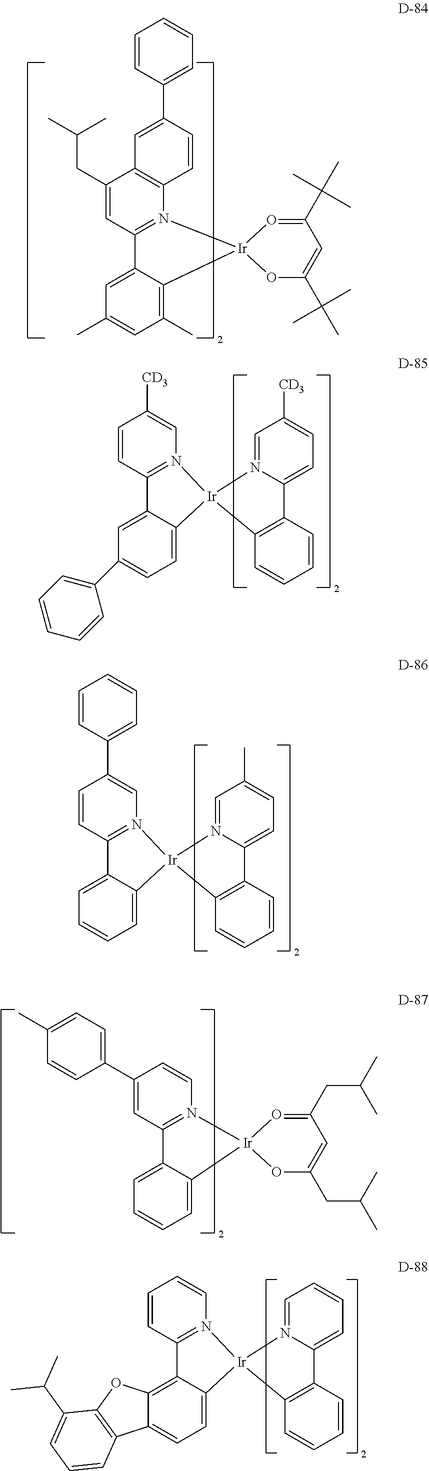

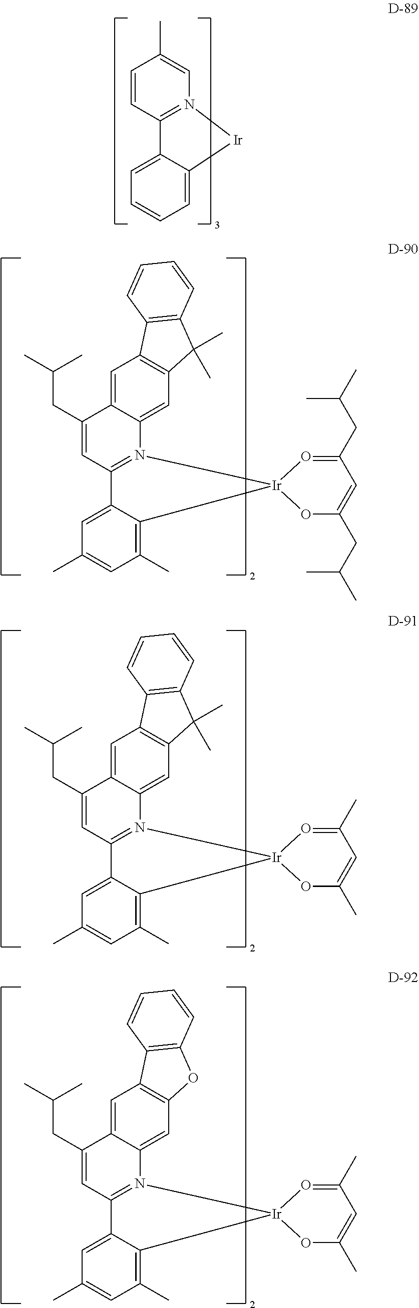

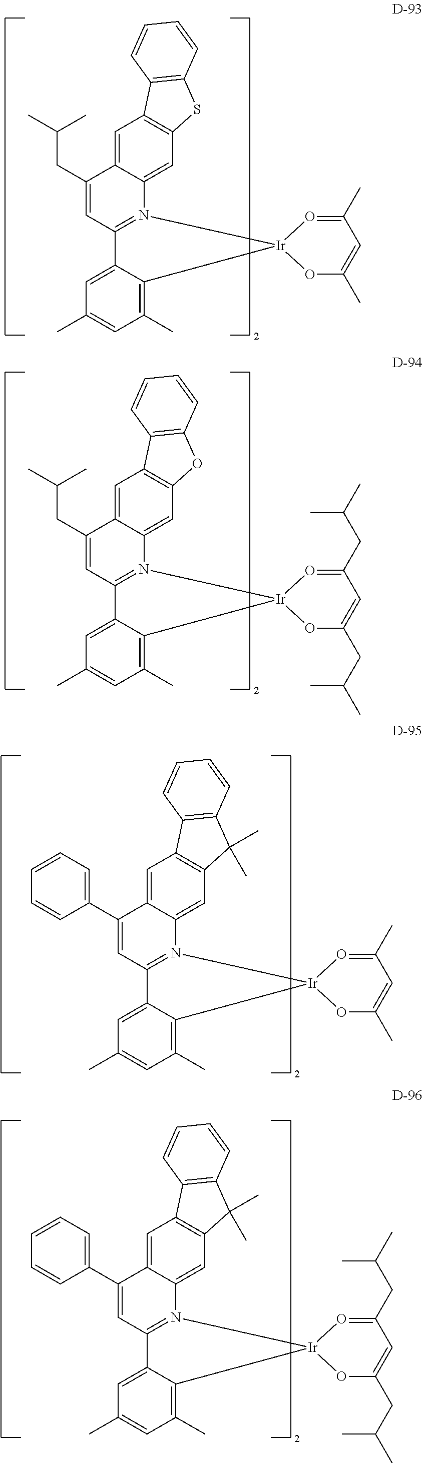

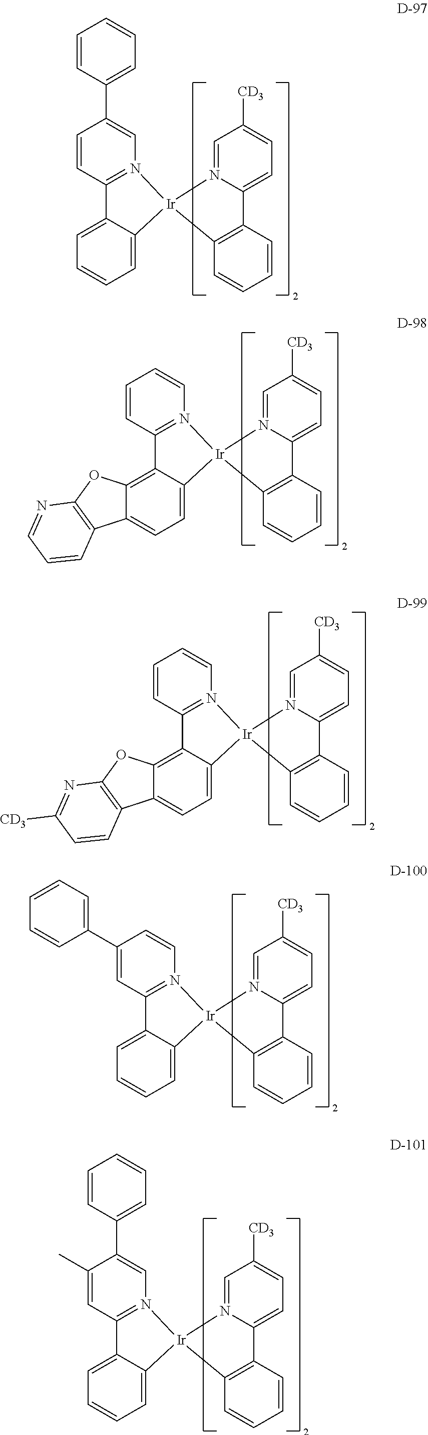

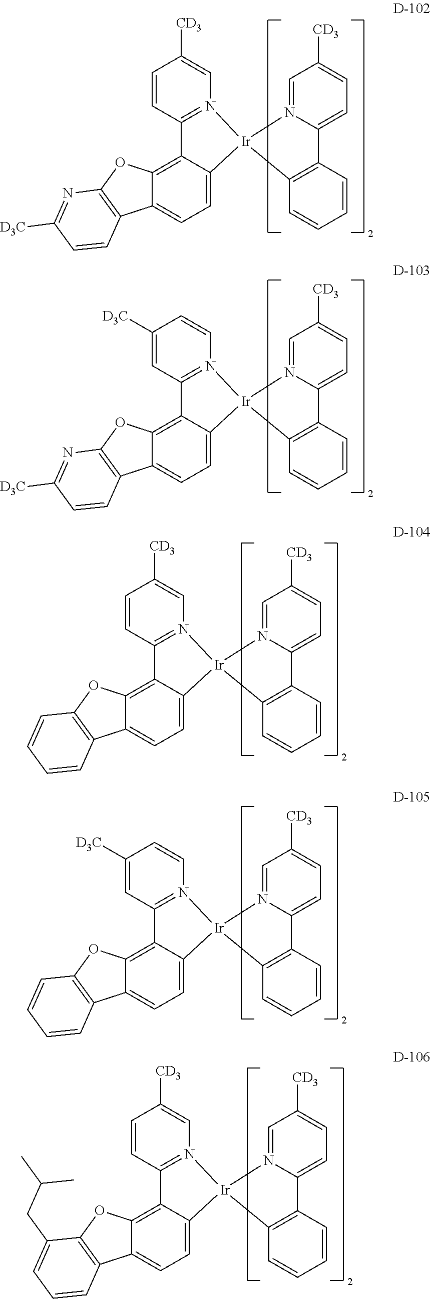

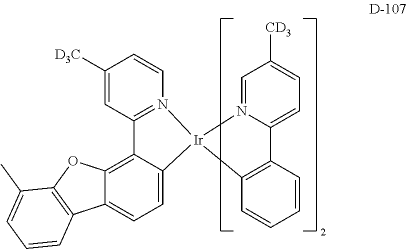

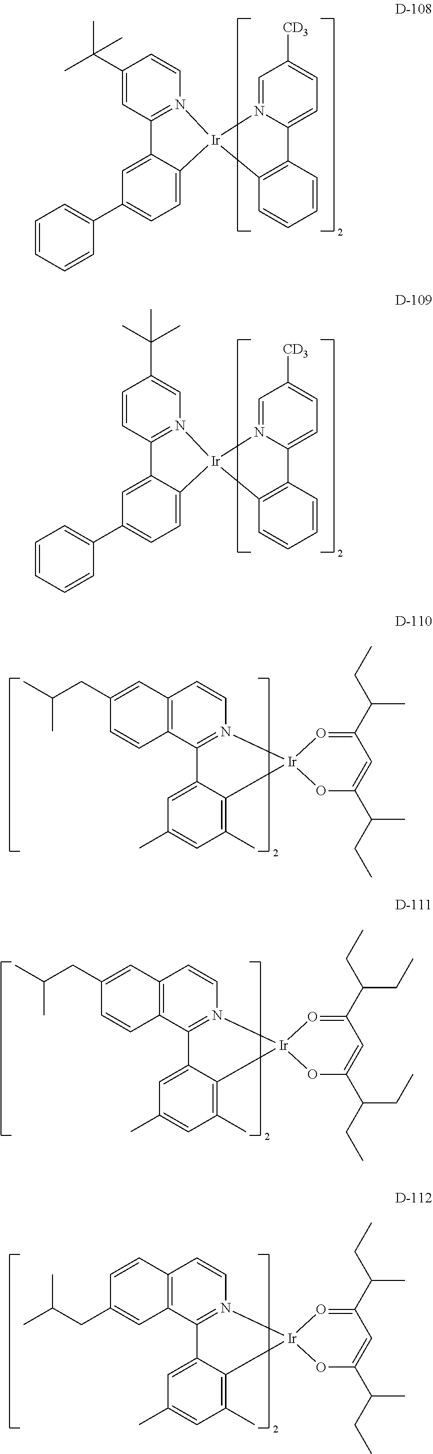

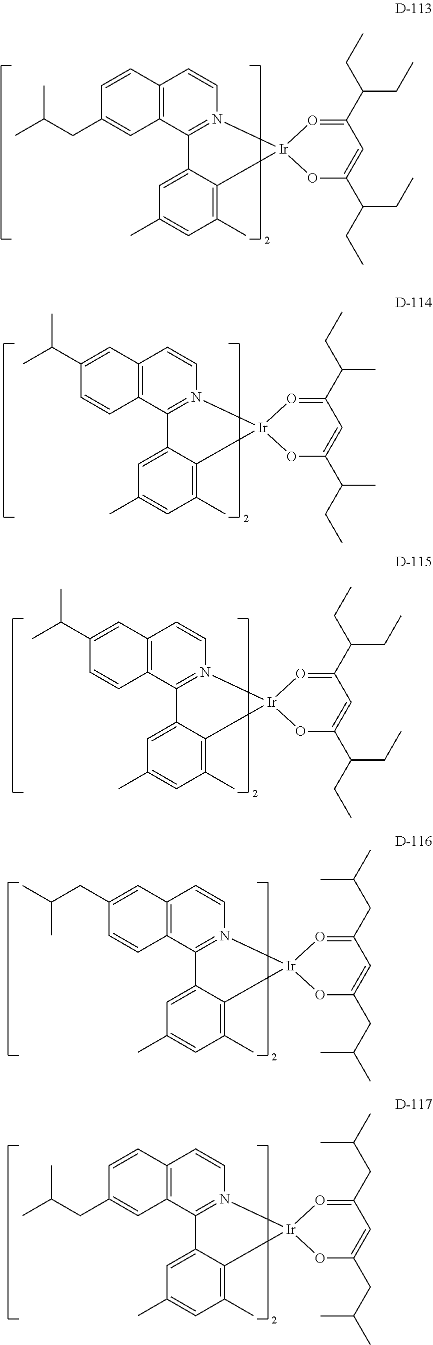

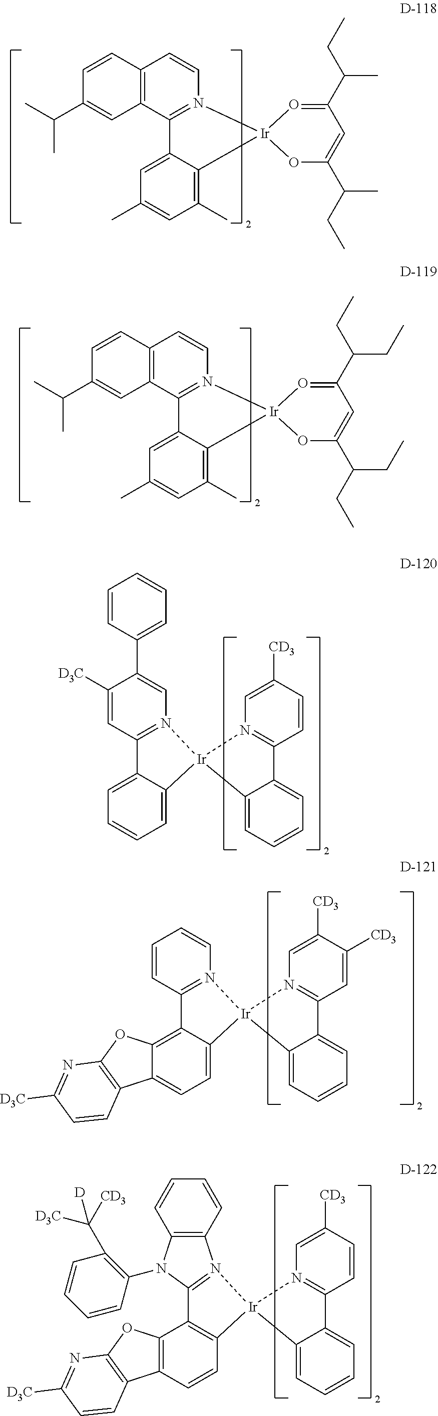

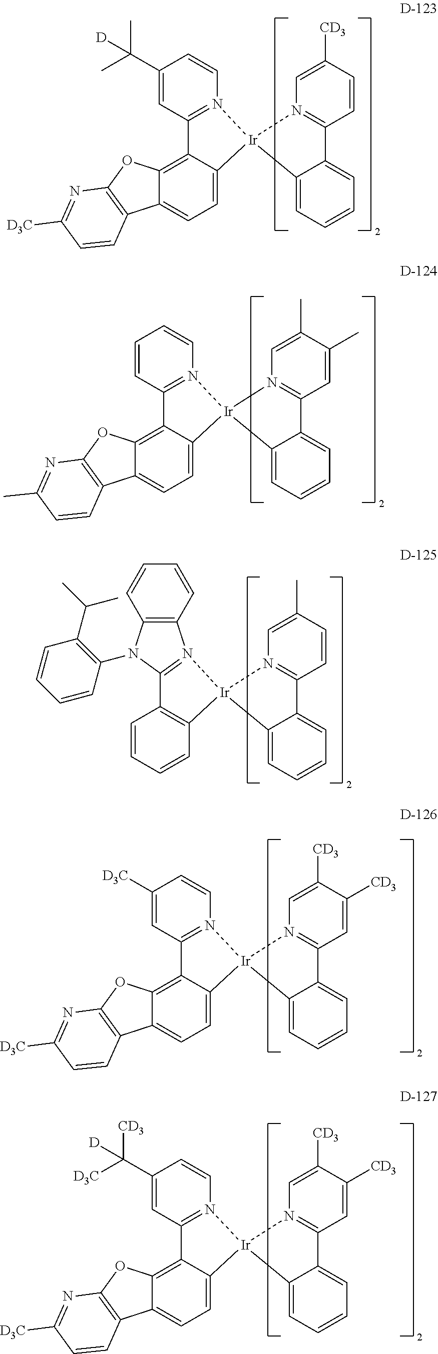

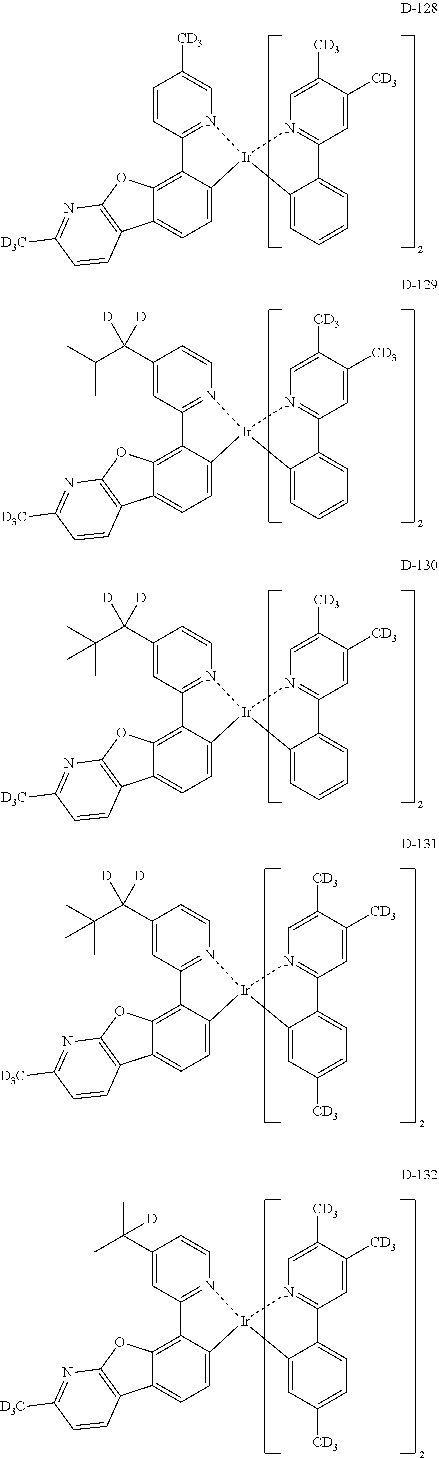

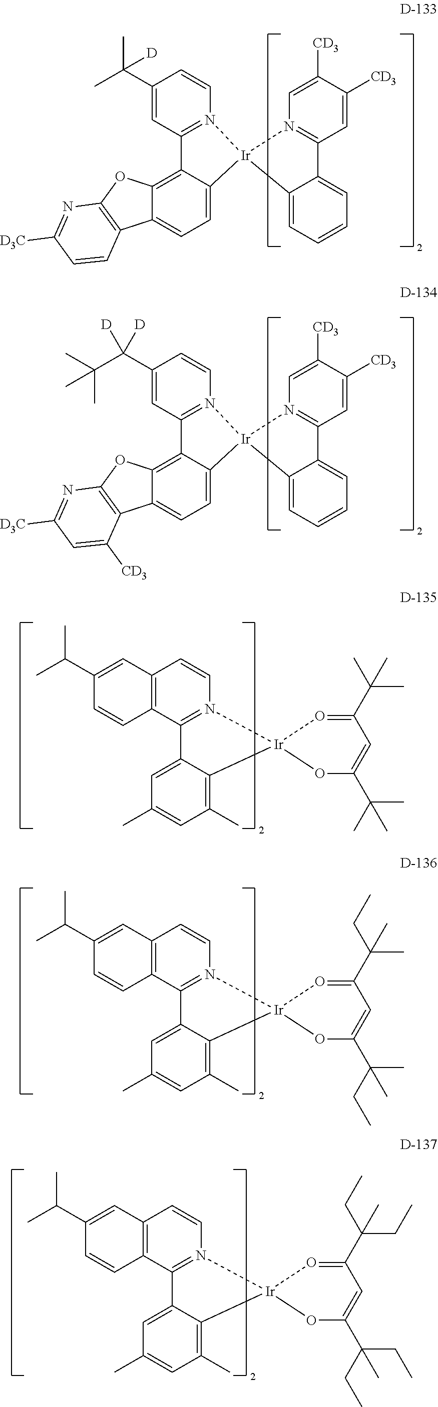

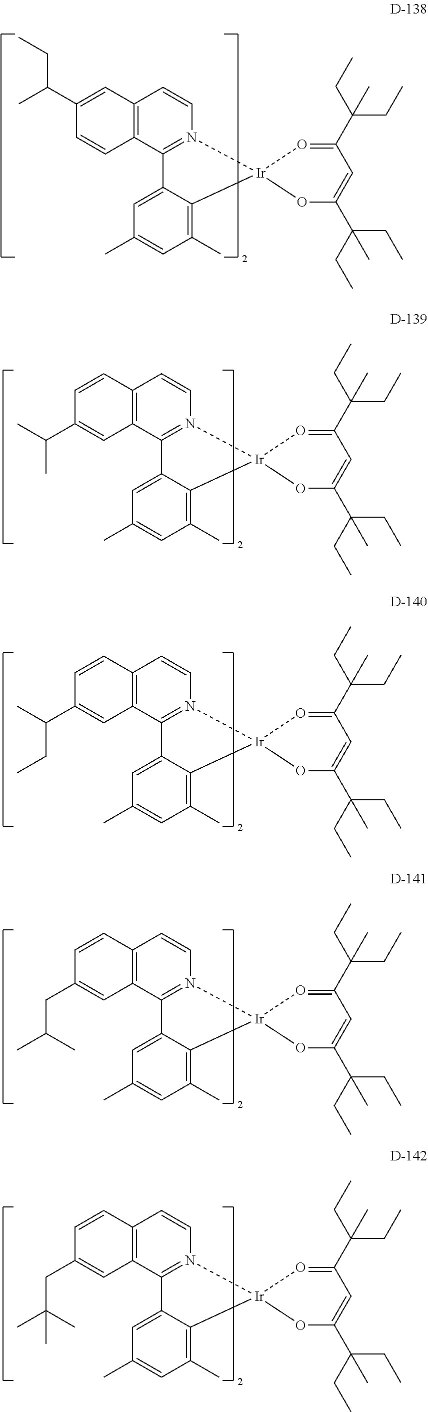

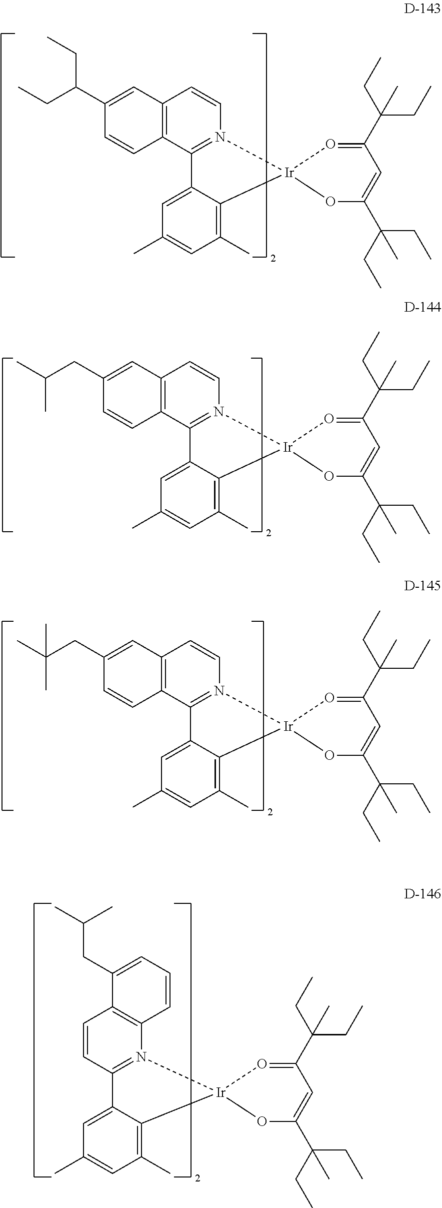

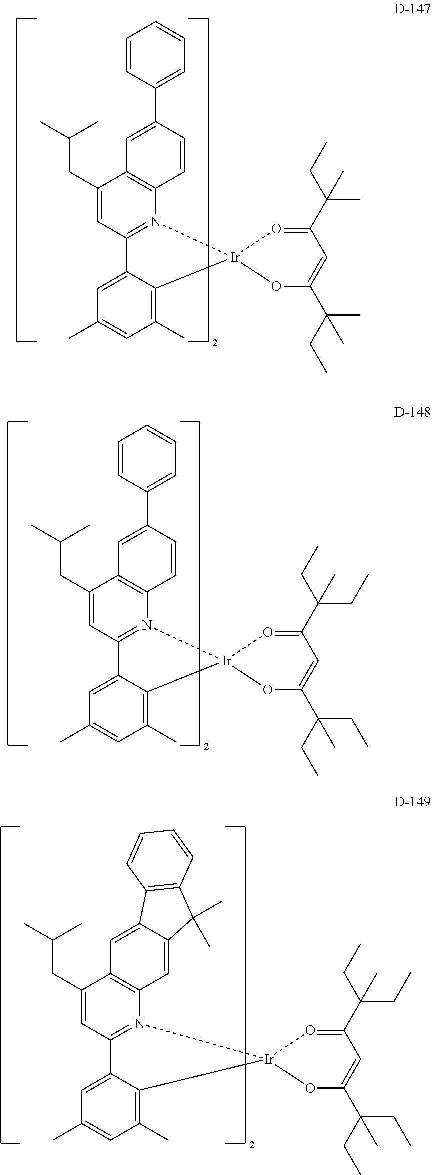

The organic electroluminescent device of the present disclosure can be manufactured by forming a first electrode or a second electrode on a substrate, and then forming an organic layer using any one of a dry deposition method such as vacuum deposition, sputtering, plasma, or ion plating, or a wet deposition method such as ink jet printing, nozzle printing, slot coating, spin coating, dip coating, or flow coating, and then forming a second electrode or a first electrode thereon. When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

When forming a layer by the organic electroluminescent material according to one embodiment, the layer can be formed by the above-listed methods, and can often be formed by co-deposition or mixture-deposition. The co-deposition is a mixed deposition method in which two or more materials are put into respective individual crucible sources, and a current is applied to both cells simultaneously to evaporate the materials; and the mixture-deposition is a mixed deposition method in which two or more materials are mixed in one crucible source before deposition, and then a current is applied to one cell to evaporate the materials.

According to one embodiment, the present disclosure can provide a display device comprising including a compound represented by Formulas 1 to 3 as an organic electroluminescent material. In addition, the organic electroluminescent device of the present disclosure can be used for the manufacture of display devices such as smartphones, tablets, notebooks, PCs, TVs, or display devices for vehicles, or lighting devices such as outdoor or indoor lighting.

### [Example 1] Synthesis of Compound H1-149

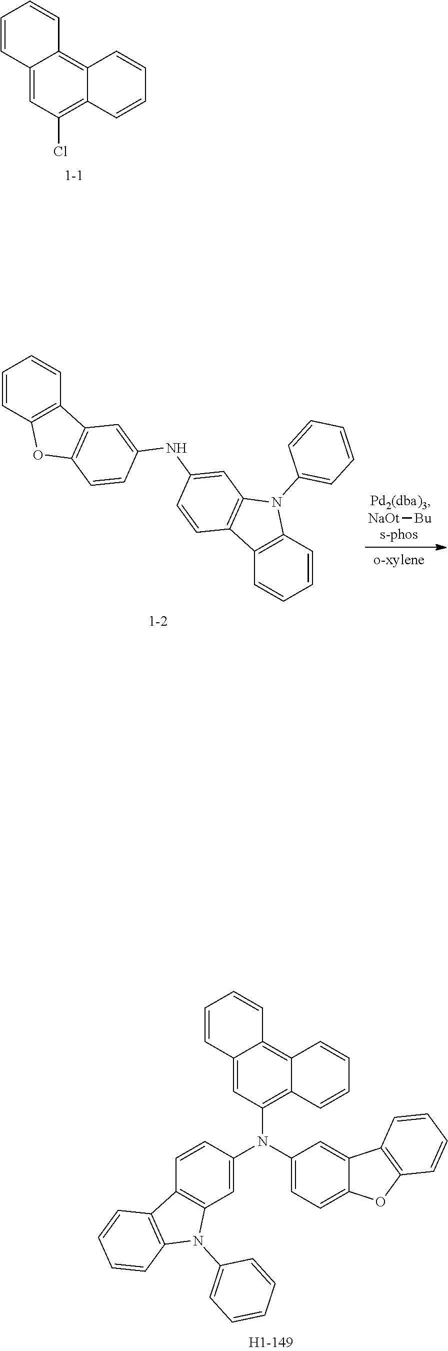

Compound 1-1 (6.6 g, 25.91 mmol), Compound 1-2 (10 g, 23.56 mmol), tris (dibenzylideneacetone) dipalladium (0) (Pd₂(dba)₃) (1.07 g, 1.178 mmol), sodium tert-butoxide (NaOt-Bu) (3.4 g, 35.34 mmol), and s-phos (0.96 g, 2.356 mmol) were added to 120 mL of o-xylene, and then stirred at 120° C. for 2 hours. After the reaction is completed, the mixture was cooled to room temperature, filtered through Celite, and distilled under reduced pressure. Next, it was separated by column chromatography to obtain compound H1-149 (7.1 g, yield: 50%).

| | MW | M.P |
|---|---|---|
| H1-149 | 600.7 | 165.3° C. |

### [Example 2] Synthesis of Compound H1-146

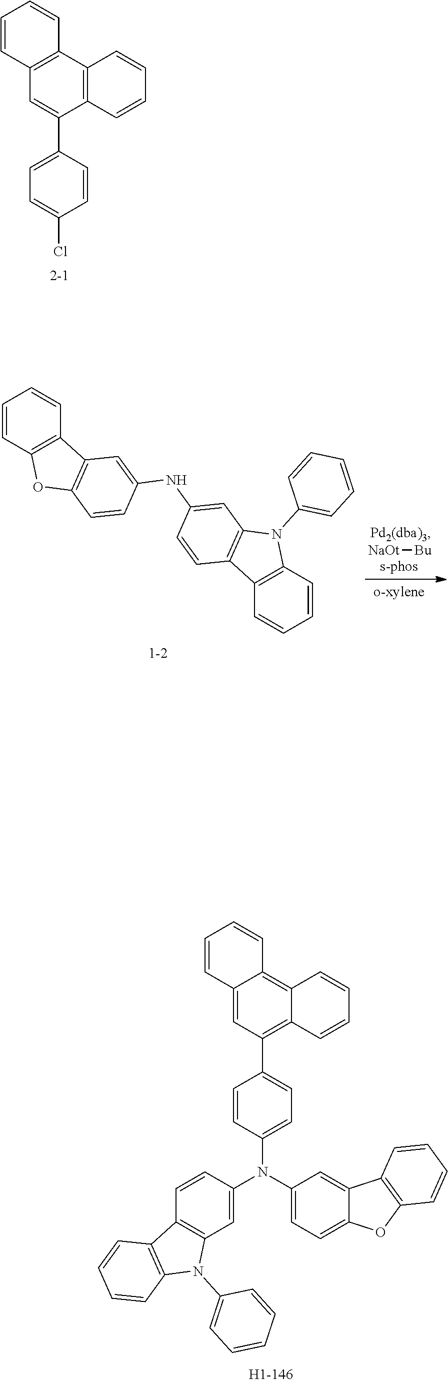

Compound 2-1 (2.4 g, 8.482 mmol), compound 1-2 (3 g, 7.068 mmol), Pd₂(dba)₃ (0.32 g, 0.353 mmol), NaOt-Bu (1 g, 10.60 mmol), and s-phos (0.29 g, 0.706 mmol) were added to 35 mL of o-xylene, and then stirred to 120° C. for 2 hours. After the reaction is completed, the mixture was cooled to room temperature, filtered through Celite, and distilled under reduced pressure. Next, it was separated by column chromatography to obtain compound H1-146 (4.2 g, yield: 87%).

| | MW | M.P |
|---|---|---|
| H1-146 | 676.8 | 268.8° C. |

### [Example 3] Synthesis of Compound H1-161

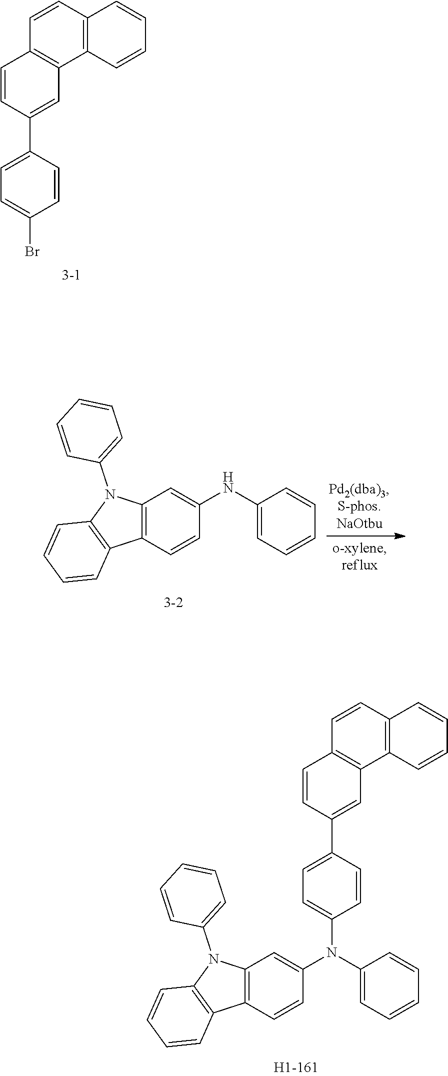 [SMILES: Brc1ccc(-c2ccc3ccc4ccccc4c3c2)cc1 ; c1ccc(Nc2ccc3c4ccccc4n(-c4ccccc4)c3c2)cc1 ; c1ccc(N(c2ccc(-c3ccc4ccc5ccccc5c4c3)cc2)c2ccc3c4ccccc4n(-c4ccccc4)c3c2)cc1]

Compound 3-1 (10.0 g, 30.0 mmol), compound 3-2 (10.53 g, 31.5 mmol), Pd₂(dba)₃ (1.4 g, 1.5 mmol), S-phos (1.2 g, 3.0 mmol), and NaOtBu (4.3 g, 45.0 mmol) were added to 150 ml of o-xylene, and then stirred under reflux at 120° C. for 1 hour. After the reaction is completed, the mixture was cooled to room temperature, the layers were separated (EA/H2O). Next, it was filtered with Celite and then with silica to create a solid. Thereafter, it was filtered to obtain compound H1-161 (2.4 g, yield: 14%).

| | MW | M.P |
|---|---|---|
| H1-161 | 586.7 | 208° C. |

### [Example 4] Synthesis of Compound H1-147

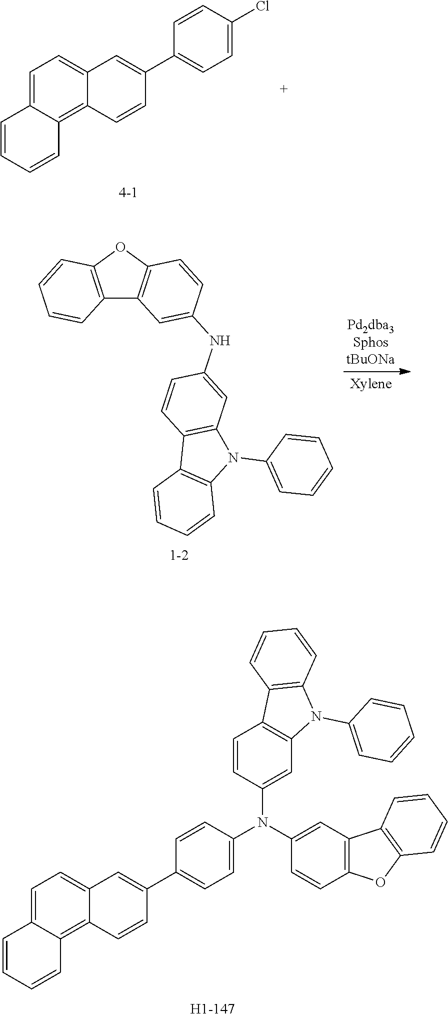 [SMILES: Clc1ccc(-c2ccc3c(ccc4ccccc43)c2)cc1 ; c1ccc(-n2c3ccccc3c3ccc(Nc4ccc5oc6ccccc6c5c4)cc32)cc1 ; c1ccc(-n2c3ccccc3c3ccc(N(c4ccc(-c5ccc6c(ccc7ccccc76)c5)cc4)c4ccc5oc6ccccc6c5c4)cc32)cc1]

Compound 4-1 (3.3 g, 11.4 mol), compound 1-2 (4.65 g, 11.4 mmol), NaOtBu (1.64 g, 17.1 mmol), S-Phos (374 mg, 0.912 mmol), Pd₂(dba)₃ (522 mg, 0.57 mmol), and 57 mL of xylene were added to a flask and then dissolved, and stirred under reflux 160° C. for 30 minutes. After the reaction is completed, the organic layer is extracted with ethyl acetate, the remaining moisture is removed using magnesium sulfate. The residue was dried and separated using column chromatography to obtain compound H1-147 (4 g, yield: 51%).

| | MW | M.P |
|---|---|---|
| H1-147 | 676.82 | 275.3° C. |

Hereinafter, the preparation method of an organic electroluminescent device comprising the compound according to the present disclosure and an organic electroluminescent material comprising the same and the device properties thereof will be explained in order to understand the present disclosure in detail.

### [Device Example 1] Preparation of an OLED Comprising the Compound According to Present Disclosure

An OLED according to the present disclosure was prepared. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and thereafter was stored in isopropyl alcohol and then used. Thereafter, the ITO substrate was mounted on a substrate holder of a vacuum vapor deposition apparatus. Compound HI-1 was then introduced into a cell of the vacuum vapor deposition apparatus, and Compound HT-1 was introduced into another cell. The two materials were evaporated at different rates, and Compound HI-1 was deposited in a doping amount of 3 wt % based on the total amount of Compounds HI-1 and HT-1 to form a hole injection layer having a thickness of 10 nm. Then, compound HT-1 was deposited on the hole injection layer to form a first hole transport layer with a thickness of 80 nm. Then, compound HT2-1 was introduced into another cell of the vacuum vapor deposition apparatus, and was evaporated by applying an electric current to the cell, thereby forming a second hole transport layer with a thickness of 55 nm on the first hole transport layer. Then, compound H1-42 was introduced as an electron blocking layer material, and an electron blocking layer with a thickness of 5 nm was deposited on the second hole transport layer. The first host compound and the second host compound described in Table 1 below as hosts were introduced into the two cells of the vacuum vapor deposition apparatus, respectively, and compound D-39 as a dopant was introduced into another cell, and then the two host materials were evaporated at a rate of 1:1 and the dopant material was evaporated at a different rate, simultaneously, and was deposited in a doping amount of 3 wt % with respect to the total amount of the hosts and the dopant to form a light-emitting layer having a thickness of 40 nm on the electron blocking layer. Next, compound ETL-1 and compound EIL-1 were evaporated as electron transport materials at a weight ratio of 50:50 to deposit an electron transport layer having a thickness of 35 nm on the light-emitting layer. Next, compound EIL-1 was deposited to for an electron injection layer having a thickness of 2 nm on the electron transport layer, and then an Al cathode having a thickness of 80 nm was deposited on the electron injection layer using another vacuum vapor deposition apparatus. Thus, an OLED was produced. Each compound used for all of the materials were purified by vacuum sublimation at 10-6 Torr.

### [Device Examples 2 to 22] Preparation of OLEDs Comprising the Compound According to Present Disclosure

OLEDs were manufactured in the same manner as Device Example 1, except that the compounds shown in Tables 1 to 4 below were used as the second hole transport layer material and the host material of the light-emitting layer.

### [Comparative Examples 1 to 22] Preparation of OLEDs which do not Include an Electron Blocking Layer

OLEDs were manufactured in the same manner as Device Example 1, except that the compounds shown in Tables 1 to 4 below were used as the second hole transport layer material and the host material of the light-emitting layer, an electron blocking layer was not included, and a second hole transport layer having a thickness of 60 nm was deposited.

The driving voltage, the luminous efficiency, and the luminous color at a luminance of 5,000 nit and the time taken for luminance to decrease from 100% to 95% at a luminance of 10,000 nit (lifespan: T95) of the OLEDs of Device Examples 1 to 22 and Comparative Examples 1 to 22 produced as described above were measured, and the results thereof are shown in Tables 1 to 4 below.

TABLE 1

| | Second Hole | Electron | | | Driving | Luminous | | |
|---|---|---|---|---|---|---|---|---|
| | Transport | Blocking | First Host | Second Host | Voltage | Efficiency | Luminous | Lifespan |
| | Layer | Layer | Compound | Compound | (V) | (cd/A) | Color | T95(hr) |
| Device | HT2-1 | H1-42 | H4-157 | H5-88 | 3.9 | 34.6 | Red | 395 |
| Example 1 | | | | | | | | |
| Comparative | HT2-1 | — | H4-157 | H5-88 | 3.7 | 34.2 | Red | 288 |
| Example 1 | | | | | | | | |
| Device | HT2-1 | H1-42 | H4-191 | H5-131 | 3.7 | 35.0 | Red | 353 |
| Example 2 | | | | | | | | |
| Comparative | HT2-1 | — | H4-191 | H5-131 | 3.6 | 33.9 | Red | 178 |
| Example 2 | | | | | | | | |
| Device | HT2-1 | H1-42 | H4-247 | H5-104 | 3.7 | 35.1 | Red | 162 |
| Example 3 | | | | | | | | |
| Comparative | HT2-1 | — | H4-247 | H5-104 | 3.7 | 32.6 | Red | 107 |
| Example 3 | | | | | | | | |
| Device | HT2-1 | H1-42 | H4-246 | H5-51 | 3.6 | 34.9 | Red | 379 |
| Example 4 | | | | | | | | |
| Comparative | HT2-1 | — | H4-246 | H5-51 | 3.5 | 33.4 | Red | 279 |
| Example 4 | | | | | | | | |
| Device | HT2-1 | H1-42 | H4-160 | H5-86 | 3.7 | 35.8 | Red | 387 |
| Example 5 | | | | | | | | |
| Comparative | HT2-1 | — | H4-160 | H5-86 | 3.6 | 35.1 | Red | 314 |
| Example 5 | | | | | | | | |

TABLE 2

| | Second Hole | Electron | | | Driving | Luminous | | |
|---|---|---|---|---|---|---|---|---|
| | Transport | Blocking | First Host | Second Host | Voltage | Efficiency | Luminous | Lifespan |
| | Layer | Layer | Compound | Compound | (V) | (cd/A) | Color | T95(hr) |
| Device | HT2-2 | H1-42 | H4-246 | H5-51 | 3.7 | 33.0 | Red | 348 |
| Example 6 | | | | | | | | |
| Comparative | HT2-2 | — | H4-246 | H5-51 | 3.5 | 32.7 | Red | 267 |
| Example 6 | | | | | | | | |
| Device | HT2-2 | H1-42 | H4-160 | H5-86 | 3.7 | 33.8 | Red | 346 |
| Example 7 | | | | | | | | |
| Comparative | HT2-2 | — | H4-160 | H5-86 | 3.6 | 33.6 | Red | 308 |
| Example 7 | | | | | | | | |
| Device | HT2-2 | H1-42 | H3-89 | H5-86 | 3.9 | 33.9 | Red | 318 |
| Example 8 | | | | | | | | |
| Comparative | HT2-2 | — | H3-89 | H5-86 | 3.8 | 33.4 | Red | 261 |
| Example 8 | | | | | | | | |
| Device | HT2-2 | H1-42 | H4-286 | H5-131 | 3.6 | 33.9 | Red | 338 |
| Example 9 | | | | | | | | |
| Comparative | HT2-2 | — | H4-286 | H5-131 | 3.5 | 32.6 | Red | 256 |
| Example 9 | | | | | | | | |
| Device | HT2-2 | H1-42 | H4-283 | H5-131 | 3.8 | 33.4 | Red | 270 |
| Example 10 | | | | | | | | |
| Comparative | HT2-2 | — | H4-283 | H5-131 | 3.7 | 32.1 | Red | 169 |
| Example 10 | | | | | | | | |
| Device | HT2-2 | H1-42 | H4-246 | H5-118 | 3.6 | 34.2 | Red | 433 |
| Example 11 | | | | | | | | |
| Comparative | HT2-2 | — | H4-246 | H5-118 | 3.5 | 32.7 | Red | 314 |
| Example 11 | | | | | | | | |
| Device | HT2-2 | H1-42 | H4-246 | H5-52 | 3.6 | 34.5 | Red | 256 |
| Example 12 | | | | | | | | |
| Comparative | HT2-2 | — | H4-246 | H5-52 | 3.4 | 32.8 | Red | 142 |
| Example 12 | | | | | | | | |
| Device | HT2-2 | H1-42 | H4-246 | H5-158 | 3.7 | 33.6 | Red | 325 |
| Example 13 | | | | | | | | |
| Comparative | HT2-2 | — | H4-246 | H5-158 | 3.6 | 33.1 | Red | 278 |
| Example 13 | | | | | | | | |

TABLE 3

| | Second Hole | Electron | | | Driving | Luminous | | |
|---|---|---|---|---|---|---|---|---|
| | Transport | Blocking | First Host | Second Host | Voltage | Efficiency | Luminous | Lifespan |
| | Layer | Layer | Compound | Compound | (V) | (cd/A) | Color | T95(hr) |
| Device | HT2-3 | H1-42 | H3-89 | H5-86 | 4.0 | 30.7 | Red | 428 |
| Example 14 | | | | | | | | |
| Comparative | HT2-3 | — | H3-89 | H5-86 | 3.8 | 30.6 | Red | 392 |
| Example 14 | | | | | | | | |
| Device | HT2-3 | H1-42 | H4-286 | H5-131 | 3.7 | 30.9 | Red | 415 |
| Example 15 | | | | | | | | |
| Comparative | HT2-3 | — | H4-286 | H5-131 | 3.6 | 30.2 | Red | 289 |
| Example 15 | | | | | | | | |
| Device | HT2-3 | H1-42 | H4-283 | H5-131 | 3.9 | 29.7 | Red | 275 |
| Example 16 | | | | | | | | |
| Comparative | HT2-3 | — | H4-283 | H5-131 | 3.8 | 30.0 | Red | 168 |
| Example 16 | | | | | | | | |
| Device | HT2-3 | H1-42 | H4-246 | H5-118 | 3.7 | 31.1 | Red | 428 |
| Example 17 | | | | | | | | |
| Comparative | HT2-3 | — | H4-246 | H5-118 | 3.6 | 29.7 | Red | 289 |
| Example 17 | | | | | | | | |
| Device | HT2-3 | H1-42 | H4-246 | H5-52 | 3.6 | 31.0 | Red | 275 |
| Example 18 | | | | | | | | |
| Comparative | HT2-3 | — | H4-246 | H5-52 | 3.5 | 29.6 | Red | 125 |
| Example 18 | | | | | | | | |
| Device | HT2-3 | H1-42 | H4-246 | H5-131 | 3.6 | 30.7 | Red | 363 |
| Example 19 | | | | | | | | |
| Comparative | HT2-3 | — | H4-246 | H5-131 | 3.5 | 29.6 | Red | 224 |
| Example 19 | | | | | | | | |

TABLE 4

| | Second Hole | Electron | | | Driving | Luminous | | |
|---|---|---|---|---|---|---|---|---|
| | Transport | Blocking | First Host | Second Host | Voltage | Efficiency | Luminous | Lifespan |
| | Layer | Layer | Compound | Compound | (V) | (cd/A) | Color | T95(hr) |
| Device | HT2-4 | H1-42 | H4-246 | H5-118 | 3.7 | 33.5 | Red | 512 |
| Example 20 | | | | | | | | |
| Comparative | HT2-4 | — | H4-246 | H5-118 | 3.6 | 31.7 | Red | 360 |
| Example 20 | | | | | | | | |
| Device | HT2-4 | H1-42 | H4-246 | H5-52 | 3.6 | 33.3 | Red | 331 |
| Example 21 | | | | | | | | |
| Comparative | HT2-4 | — | H4-246 | H5-52 | 3.5 | 31.4 | Red | 86 |
| Example 21 | | | | | | | | |
| Device | HT2-4 | H1-42 | H4-246 | H5-51 | 3.6 | 33.1 | Red | 445 |
| Example 22 | | | | | | | | |
| Comparative | HT2-4 | — | H4-246 | H5-51 | 3.5 | 31.4 | Red | 254 |
| Example 22 | | | | | | | | |

From Tables 1 to 4 above, it can be confirmed that organic electroluminescent devices (Device Examples 1 to 22) including a compound according to the present disclosure as an electron blocking layer material and including a specific combination of compounds according to the present disclosure as a host material exhibit luminous efficiency equivalent to or greater than that of organic electroluminescent devices (Comparative Examples 1 to 22) that do not include an electron blocking layer, and in particular, have significantly improved lifespan characteristics.

### [Device Examples 23 to 28] Preparation of OLEDs Comprising the Compound According to Present Disclosure

OLEDs were manufactured in the same manner as Device Example 1, except that the compounds shown in Table 5 below were used as the second hole transport layer material and the host material of the light-emitting layer, an electron blocking layer was not included, and the second hole transport layer having a thickness of 60 nm was deposited.

### [Comparative Example 23] Preparation of an OLED which does not Include an Electron Blocking Layer

An OLED was manufactured in the same manner as Device Example 1, except that the compounds shown in Table 5 below were used as the second hole transport layer material and the host material of the light-emitting layer, an electron blocking layer is not included, and the second hole transport layer having a thickness of 60 nm was deposited; in addition, the second host compound described in Table 5 was introduced into one cell within the vacuum vapor deposition apparatus, and compound D-39 as a dopant was introduced into another cell, and then deposited in a doping amount of 3 wt % based on the total amount of the hosts and the dopant to form an light-emitting layer having a thickness of 40 nm on the second hole transport layer.

The driving voltage, the luminous efficiency, and the luminous color at a luminance of 5,000 nit and the time taken for luminance to decrease from 100% to 95% at a luminance of 10,000 nit (lifespan: T95) of the OLEDs of Device Examples 23 to 28 and Comparative Example 23 produced as described above were measured, and the results thereof are shown in Table 5 below.

TABLE 5

| | Second Hole | | | Driving | Luminous | | |
|---|---|---|---|---|---|---|---|
| | Transport | First Host | Second Host | Voltage | Efficiency | Luminous | Lifespan |
| | Layer | Compound | Compound | (V) | (cd/A) | Color | T95(hr) |
| Device | HT2-5 | H1-132 | H5-86 | 3.8 | 32.1 | Red | 350 |
| Example 23 | | | | | | | |
| Device | HT2-5 | H1-135 | H5-86 | 3.7 | 32.7 | Red | 414 |
| Example 24 | | | | | | | |
| Device | HT2-5 | H1-147 | H5-86 | 3.6 | 31.7 | Red | 353 |
| Example 25 | | | | | | | |
| Device | HT2-2 | H1-161 | H5-86 | 3.7 | 33.0 | Red | 789 |
| Example 26 | | | | | | | |
| Device | HT2-2 | H1-146 | H5-86 | 3.7 | 32.9 | Red | 459 |
| Example 27 | | | | | | | |
| Device | HT2-2 | H1-149 | H5-86 | 3.7 | 32.2 | Red | 170 |
| Example 28 | | | | | | | |
| Comparative | HT2-5 | — | H5-86 | 4.5 | 26.6 | Red | 25 |
| Example 23 | | | | | | | |

From Table 5 above, it can be confirmed that the organic electroluminescent devices (Device Examples 23 to 28) including a specific combination of compounds according to the present disclosure as a light-emitting layer material exhibit lower driving voltage and higher luminous efficiency, and in particular, significantly improved lifespan characteristics, compared to Comparative Example 23.

The compounds used in Device Examples and Comparative Examples are specifically shown in Table 6 below.

TABLE 6

| | |
|---|---|
| Hole Injection Layer/ First Hole Transport Layer | 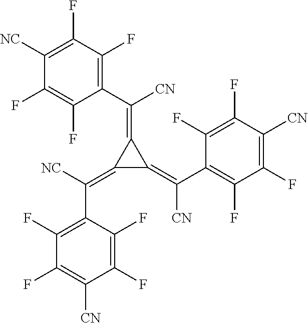 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | HI-1 |
| | 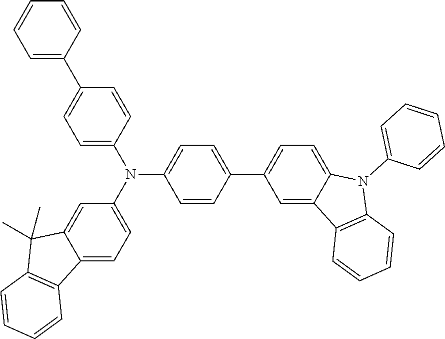 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21] |
| | HT-1 |
| Second Hole Transport Layer | 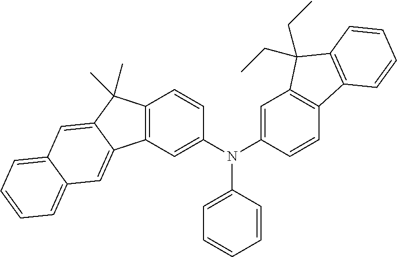 [SMILES: CCC1(CC)c2ccccc2-c2ccc(N(c3ccccc3)c3ccc4c(c3)-c3cc5ccccc5cc3C4(C)C)cc21] |
| | HT-2-1 |
| | 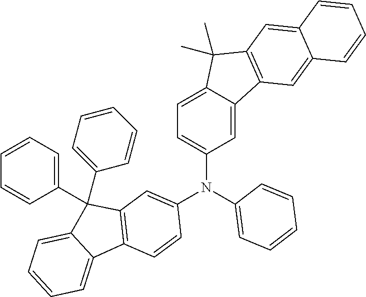 [SMILES: CC1(C)c2ccc(N(c3ccccc3)c3ccc4c(c3)C(c3ccccc3)(c3ccccc3)c3ccccc3-4)cc2-c2cc3ccccc3cc21] |
| | HT-2-2 |
| | 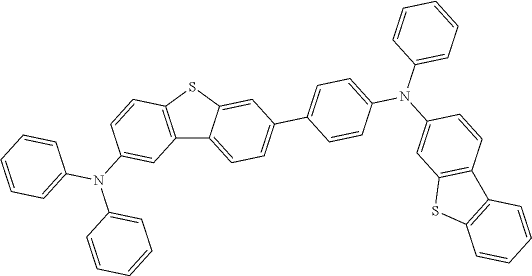 [SMILES: c1ccc(N(c2ccc(-c3ccc4c(c3)sc3ccc(N(c5ccccc5)c5ccccc5)cc34)cc2)c2ccc3c(c2)sc2ccccc23)cc1] |
| | HT-2-3 |
| | 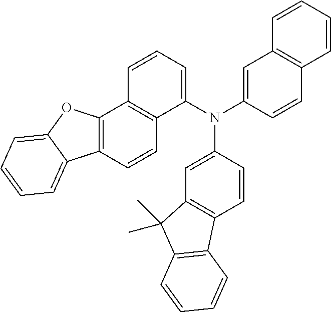 [SMILES: CC1(C)c2ccccc2-c2ccc(N(c3ccc4ccccc4c3)c3cccc4c3ccc3c5ccccc5oc43)cc21] |
| | HT-2-4 |
| | 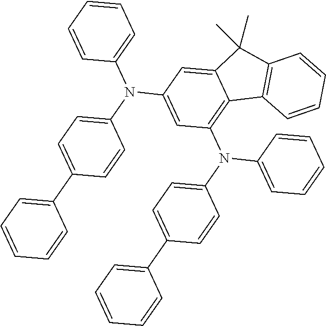 [SMILES: CC1(C)c2ccccc2-c2c(N(c3ccccc3)c3ccc(-c4ccccc4)cc3)cc(N(c3ccccc3)c3ccc(-c4ccccc4)cc3)cc21] |
| | HT-2-5 |
| Electron Blocking Layer | 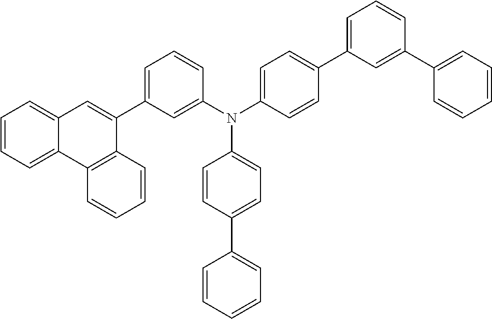 [SMILES: c1ccc(-c2ccc(N(c3ccc(-c4cccc(-c5ccccc5)c4)cc3)c3cccc(-c4cc5ccccc5c5ccccc45)c3)cc2)cc1] |
| | H1-42 |
| Light- Emitting Layer | 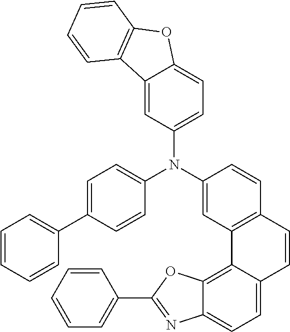 [SMILES: c1ccc(-c2ccc(N(c3ccc4oc5ccccc5c4c3)c3ccc4ccc5ccc6nc(-c7ccccc7)oc6c5c4c3)cc2)cc1] |
| | H4-157 |
| | 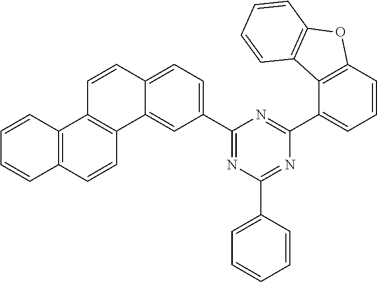 [SMILES: c1ccc(-c2nc(-c3ccc4ccc5c6ccccc6ccc5c4c3)nc(-c3cccc4oc5ccccc5c34)n2)cc1] |
| | H5-88 |
| | 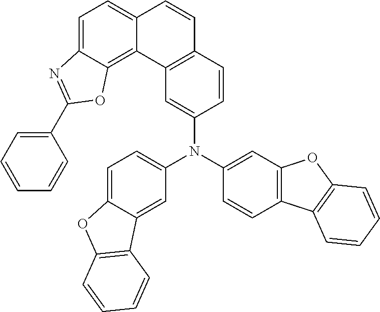 [SMILES: c1ccc(-c2nc3ccc4ccc5ccc(N(c6ccc7c(c6)oc6ccccc67)c6ccc7oc8ccccc8c7c6)cc5c4c3o2)cc1] |
| | H4-191 |
| | 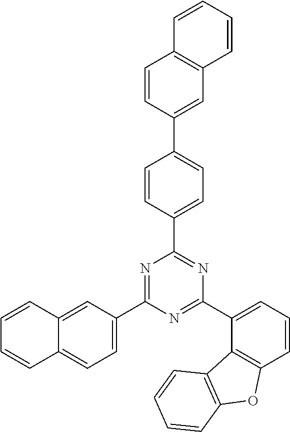 [SMILES: c1ccc2cc(-c3ccc(-c4nc(-c5ccc6ccccc6c5)nc(-c5cccc6oc7ccccc7c56)n4)cc3)ccc2c1] |
| | H5-131 |
| | 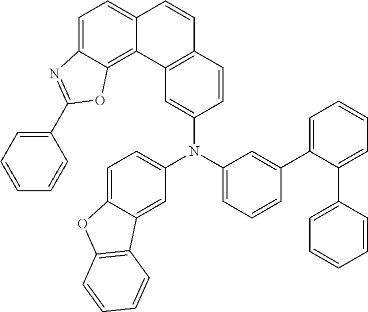 [SMILES: c1ccc(-c2nc3ccc4ccc5ccc(N(c6cccc(-c7ccccc7-c7ccccc7)c6)c6ccc7oc8ccccc8c7c6)cc5c4c3o2)cc1] |
| | H4-247 |
| | 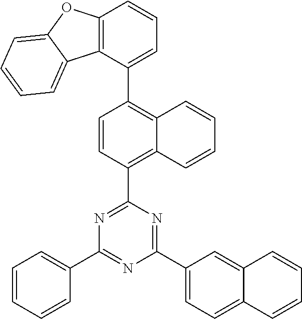 [SMILES: c1ccc(-c2nc(-c3ccc4ccccc4c3)nc(-c3ccc(-c4cccc5oc6ccccc6c45)c4ccccc34)n2)cc1] |
| | H5-104 |
| | 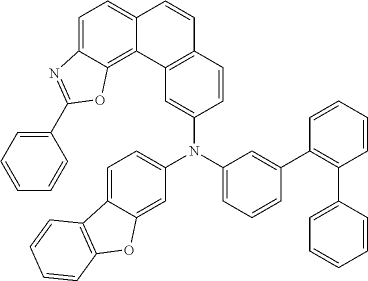 [SMILES: c1ccc(-c2nc3ccc4ccc5ccc(N(c6cccc(-c7ccccc7-c7ccccc7)c6)c6ccc7c(c6)oc6ccccc67)cc5c4c3o2)cc1] |
| | H4-246 |
| | 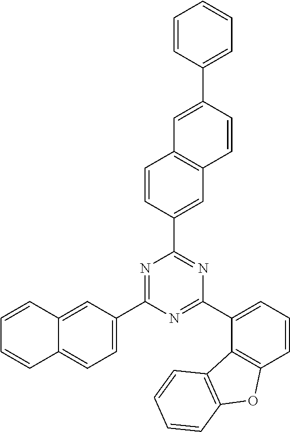 [SMILES: c1ccc(-c2ccc3cc(-c4nc(-c5ccc6ccccc6c5)nc(-c5cccc6oc7ccccc7c56)n4)ccc3c2)cc1] |
| | H5-51 |
| | 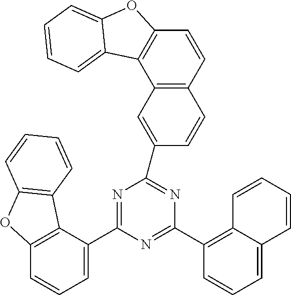 [SMILES: c1ccc2c(-c3nc(-c4ccc5ccc6oc7ccccc7c6c5c4)nc(-c4cccc5oc6ccccc6c45)n3)cccc2c1] |
| | H4-160 |
| | 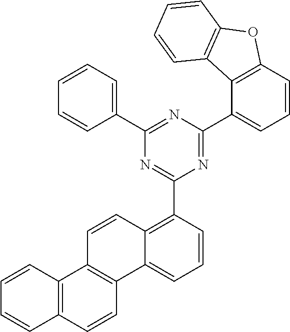 [SMILES: c1ccc(-c2nc(-c3cccc4c3ccc3c5ccccc5ccc43)nc(-c3cccc4oc5ccccc5c34)n2)cc1] |
| | H5-86 |
| | 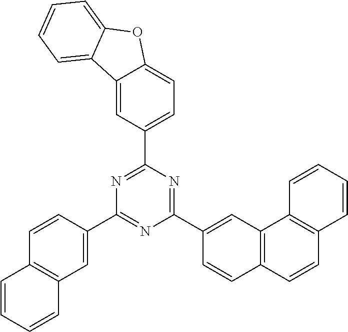 [SMILES: c1ccc2cc(-c3nc(-c4ccc5ccc6ccccc6c5c4)nc(-c4ccc5oc6ccccc6c5c4)n3)ccc2c1] |
| | H5-158 |
| | 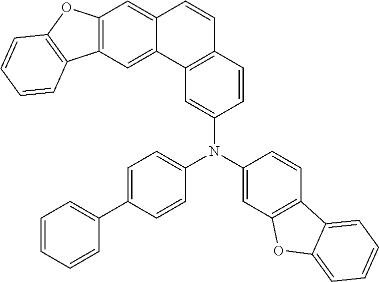 [SMILES: c1ccc(-c2ccc(N(c3ccc4c(c3)oc3ccccc34)c3ccc4ccc5cc6oc7ccccc7c6cc5c4c3)cc2)cc1] |
| | H3-89 |
| | 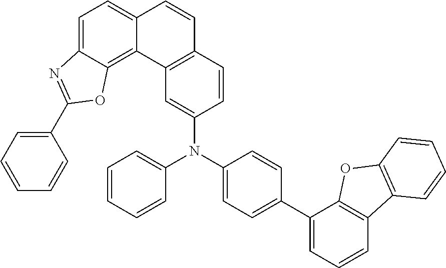 [SMILES: c1ccc(-c2nc3ccc4ccc5ccc(N(c6ccccc6)c6ccc(-c7cccc8c7oc7ccccc78)cc6)cc5c4c3o2)cc1] |
| | H4-286 |
| | 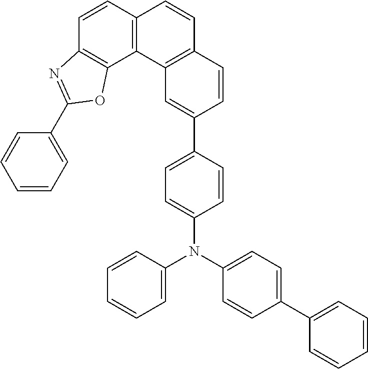 [SMILES: c1ccc(-c2ccc(N(c3ccccc3)c3ccc(-c4ccc5ccc6ccc7nc(-c8ccccc8)oc7c6c5c4)cc3)cc2)cc1] |
| | H4-283 |
| | 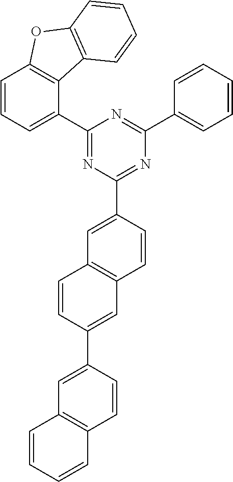 [SMILES: c1ccc(-c2nc(-c3ccc4cc(-c5ccc6ccccc6c5)ccc4c3)nc(-c3cccc4oc5ccccc5c34)n2)cc1] |
| | H5-118 |
| | 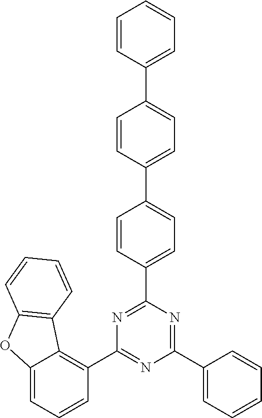 [SMILES: c1ccc(-c2ccc(-c3ccc(-c4nc(-c5ccccc5)nc(-c5cccc6oc7ccccc7c56)n4)cc3)cc2)cc1] |
| | H5-52 |
| | 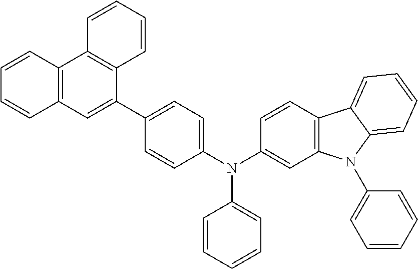 [SMILES: c1ccc(N(c2ccc(-c3cc4ccccc4c4ccccc34)cc2)c2ccc3c4ccccc4n(-c4ccccc4)c3c2)cc1] |
| | H1-132 |
| | 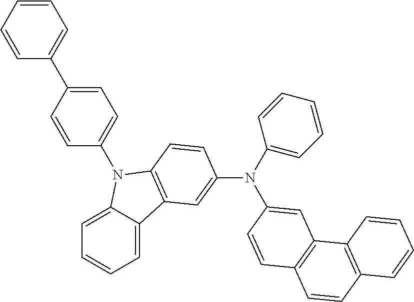 [SMILES: c1ccc(-c2ccc(-n3c4ccccc4c4cc(N(c5ccccc5)c5ccc6ccc7ccccc7c6c5)ccc43)cc2)cc1] |
| | H1-135 |
| | 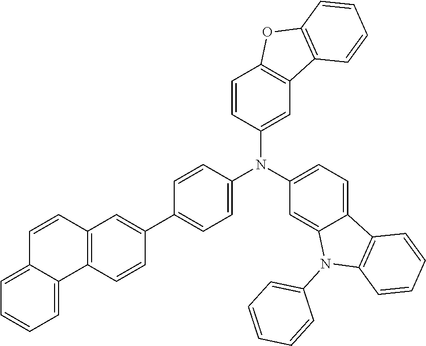 [SMILES: c1ccc(-n2c3ccccc3c3ccc(N(c4ccc(-c5ccc6c(ccc7ccccc76)c5)cc4)c4ccc5oc6ccccc6c5c4)cc32)cc1] |
| | H1-147 |
| | 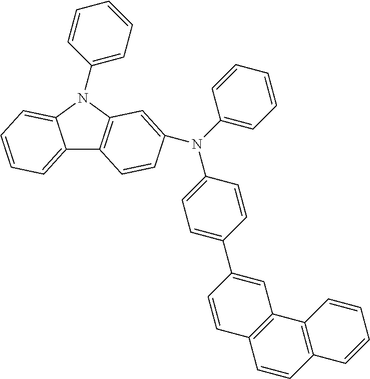 [SMILES: c1ccc(N(c2ccc(-c3ccc4ccc5ccccc5c4c3)cc2)c2ccc3c4ccccc4n(-c4ccccc4)c3c2)cc1] |
| | H1-161 |
| | 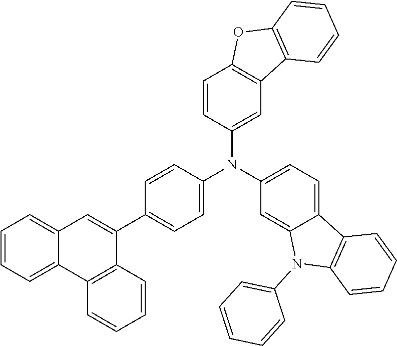 [SMILES: c1ccc(-n2c3ccccc3c3ccc(N(c4ccc(-c5cc6ccccc6c6ccccc56)cc4)c4ccc5oc6ccccc6c5c4)cc32)cc1] |
| | H1-146 |
| | 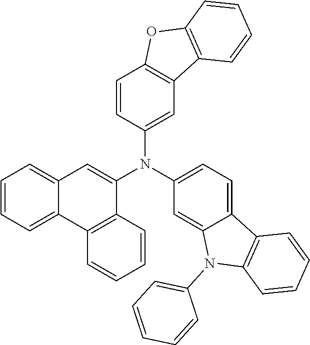 [SMILES: c1ccc(-n2c3ccccc3c3ccc(N(c4ccc5oc6ccccc6c5c4)c4cc5ccccc5c5ccccc45)cc32)cc1] |
| | H1-149 |
| | 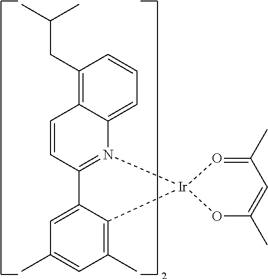 |
| | D-39 |
| Electron Transport Layer Electron Injection Layer | 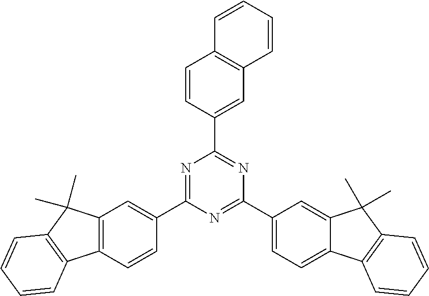 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | ETL-1 |
| | 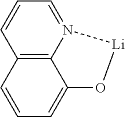 |
| | EIL-1 |

1. An organic electroluminescent device comprising:
a first electrode;
a second electrode;
a light-emitting layer between the first electrode and the second electrode; and
at least one electron blocking layer between the first electrode and the light-emitting layer, wherein the electron blocking layer comprises a compound represented by the following Formula 1, and the light-emitting layer comprises at least two kinds of compounds: 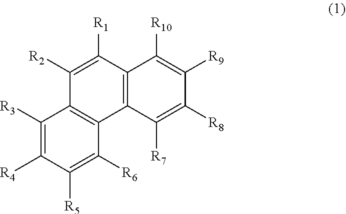
wherein,
R₁ to R₁₀ each independently represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring of (C3-C30) aliphatic ring and (C6-C30) aromatic ring, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or *-L₁-N(Ar₁)(Ar₂); provided that at least one of R₁ to R₁₀ is *-L₁-N(Ar₁)(Ar₂);
L₁ represents a single bond, a substituted or unsubstituted (C6-C30)arylene, or a substituted or unsubstituted (3- to 30-membered)heteroarylene; and
Ar₁ and Ar₂ each independently represent a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl.

2. The organic electroluminescent device according to claim 1, wherein the organic electroluminescent device comprises at least one hole auxiliary layer between the first electrode and the electron-blocking layer.

3. The organic electroluminescent device according to claim 1, wherein the light-emitting layer comprises at least one compound represented by the following Formula 2 and at least one compound represented by the following Formula 3: 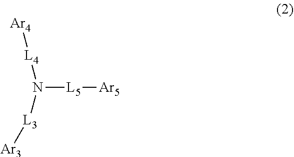
wherein
L₃ to L₅ each independently represent a single bond, a substituted or unsubstituted (C1-C30)alkylene, a substituted or unsubstituted (C6-C30)arylene, a substituted or unsubstituted (3-to 30-membered)heteroarylene, or a substituted or unsubstituted (C3-C30)cycloalkylene;
Ar₃ to Ar₅ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or —N(Ar₁₁)(Ar₁₂); and
Ar₁₁ and Ar₁₂ each independently represent a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl;
provided that, in Formula 2, the case where all of L₃ to L₅ are a single bond, and all of Ar₃ to Ar₅ are hydrogen is excluded, 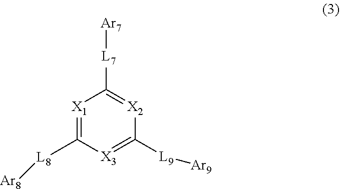
wherein,
X₁ to X₃ each independently represent N or CR₁₁; provided that at least one of X₁ to X₃ is N;
R₁₁ represents hydrogen, deuterium, a halogen, a cyano, a substituted or unsubstituted (C1-C30)alkyl, a substituted or unsubstituted (C6-C30)aryl, a substituted or unsubstituted (3-to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring of (C3-C30) aliphatic ring and (C6-C30)aromatic ring;
L₇ to L₉ each independently represent a single bond, a substituted or unsubstituted (C6-C30)arylene, or a substituted or unsubstituted (3- to 30-membered)heteroarylene;
Ar₇ to Ar₉ each independently represent a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring of (C3-C30) aliphatic ring and (C6-C30)aromatic ring, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₂₁-N(Ar₂₁)(Ar₂₂);
L₂₁ represents a single bond, a substituted or unsubstituted (C6-C30)arylene, or a substituted or unsubstituted (3- to 30-membered)heteroarylene; and
Ar₂₁ and Ar₂₂ each independently represent a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl.

4. The organic electroluminescent device according to claim 3, wherein at least one of Ar₃ to Ar₅ in Formula 2 is represented by any one of the following Formulas 2-1 to 2-3: 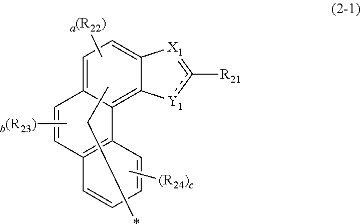
wherein
X₁ and Y₁ each independently represent —N═, —NR₂₅—, —O—, or —S—; provided that any one of X₁ and Y₁ is —N═, and the other of X₁ and Y₁ is —NR₂₅—, —O—, or —S—;
R₂₁ represents a connection position connected to L₃ to L₅ in Formula 2, or a substituted or unsubstituted (C6-C30)aryl or a substituted or unsubstituted (3- to 30-membered)heteroaryl;
R₂₂ to R₂₅ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to the adjacent substituents to form a ring(s);
a and b each independently represent an integer of 1 or 2, and c represents an integer of 1 to 3;
and when a to c are an integer of 2 or more, each of R₂₂ to each of R₂₄ may be the same as or different from each other;
* indicates a connection position connected to L₃ to L₅ in Formula 2; 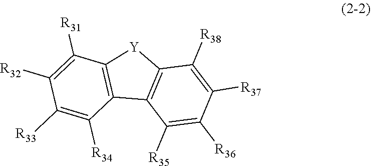
wherein
Y represents —O—, —S—, or —NR₃₉;
R₃₉ represents a substituted or unsubstituted (C6-C30)aryl, or a substituted or unsubstituted (3- to 30-membered)heteroaryl; and
R₃₁ to R₃₈ each independently represent a connection position connected to L₃ to L₅ in Formula 2; or hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to the adjacent substituents to form a ring(s); 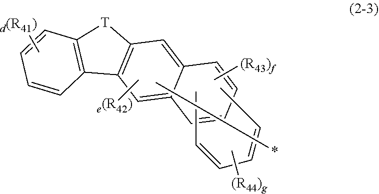
wherein
T represents —O—, —S—, —CR₄₅R₄₆, —NR₄₇, or —Se—;
R₄₅ to R₄₇ each independently represent hydrogen, deuterium, a halogen, a substituted or unsubstituted (C1-C30)alkyl, or a substituted or unsubstituted (C6-C30)aryl;
R₄₁ to R₄₄ each independently represent hydrogen, deuterium, a halogen, a substituted or unsubstituted (C1-C30)alkyl, a substituted or unsubstituted (C6-C30)aryl, a substituted or unsubstituted (3- to 30-membered)heteroaryl, or —N(Ar₂₁)(Ar₂₂); or may be linked to the adjacent substituents to form a ring(s);
Ar₂₁ and Ar₂₂ each independently represent a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl;
d and g each independently represent an integer of 1 to 4, and e and f each independently represent an integer of 1 or 2;
when d to g are an integer of 2 or more, each of R₄₁ to each of R₄₄ may be the same as or different from each other; and
* indicates a connection position connected to L₃ to L₅ in Formula 2.

5. The organic electroluminescent device according to claim 3, wherein at least one of Ar₇ to Ar₉ in Formula 3 is represented by any one of the following Formulas 2-2, 3-1, and 3-2: 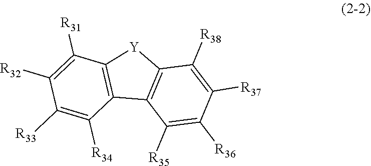
wherein
Y represents —O—, —S—, or —NR₃₉;
R₃₉ represents a substituted or unsubstituted (C6-C30)aryl, or a substituted or unsubstituted (3- to 30-membered)heteroaryl; and
R₃₁ to R₃₈ each independently represent a connection position connected to L₇ to L₉ in Formula 3; or hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to the adjacent substituents to form a ring(s); 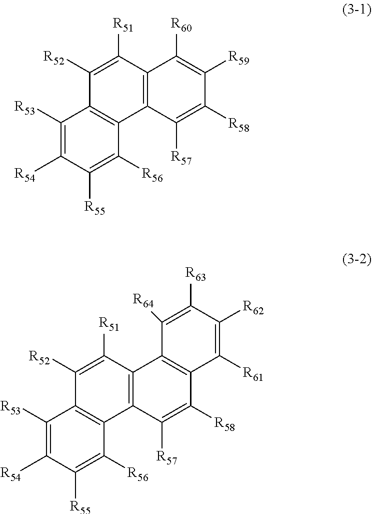
wherein
R₅₁ to R₆₄ each independently represent a connection position connected to L₇ to L₉ in Formula 3; or hydrogen, deuterium, a halogen, a cyano, a substituted or unsubstituted (C1-C30)alkyl, a substituted or unsubstituted (C3-C30)cycloalkyl, a substituted or unsubstituted (3-to 7-membered)heterocycloalkyl, a substituted or unsubstituted fused ring of (C3-C30) aliphatic ring and (C6-C30) aromatic ring, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino.

6. The organic electroluminescent device according to claim 1, wherein the compound represented by Formula 1 is selected from the following compounds:

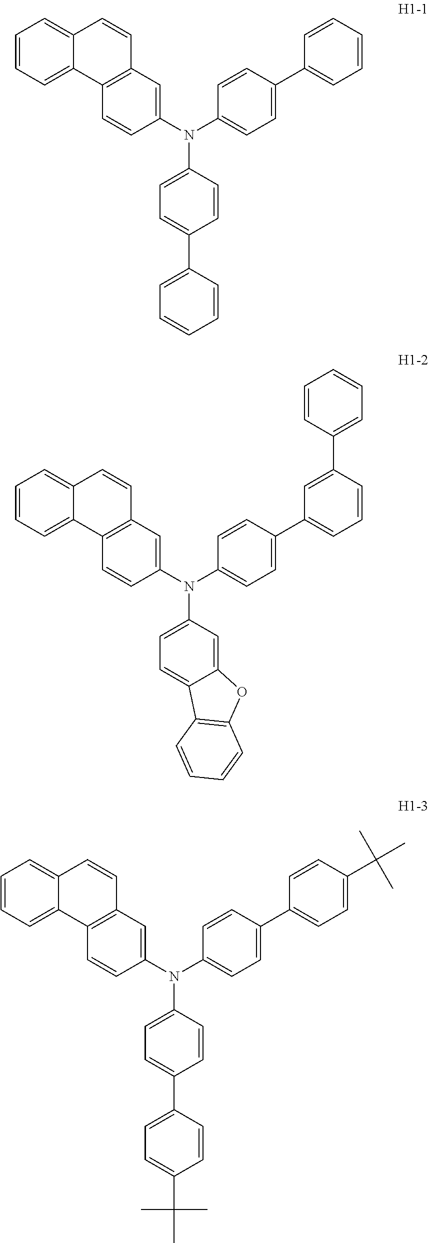 [SMILES: c1ccc(-c2ccc(N(c3ccc(-c4ccccc4)cc3)c3ccc4c(ccc5ccccc54)c3)cc2)cc1 ; c1ccc(-c2cccc(-c3ccc(N(c4ccc5c(ccc6ccccc65)c4)c4ccc5c(c4)oc4ccccc45)cc3)c2)cc1 ; CC(C)(C)c1ccc(-c2ccc(N(c3ccc(-c4ccc(C(C)(C)C)cc4)cc3)c3ccc4c(ccc5ccccc54)c3)cc2)cc1]

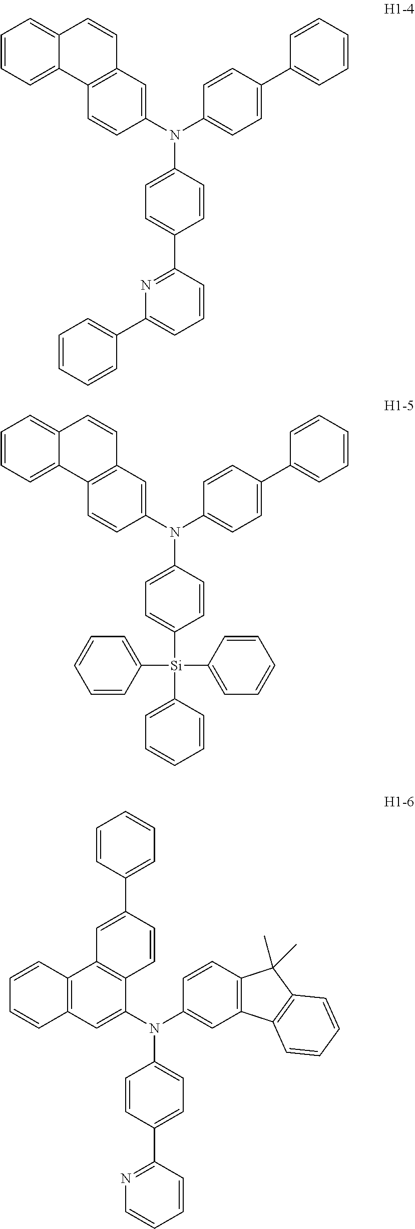

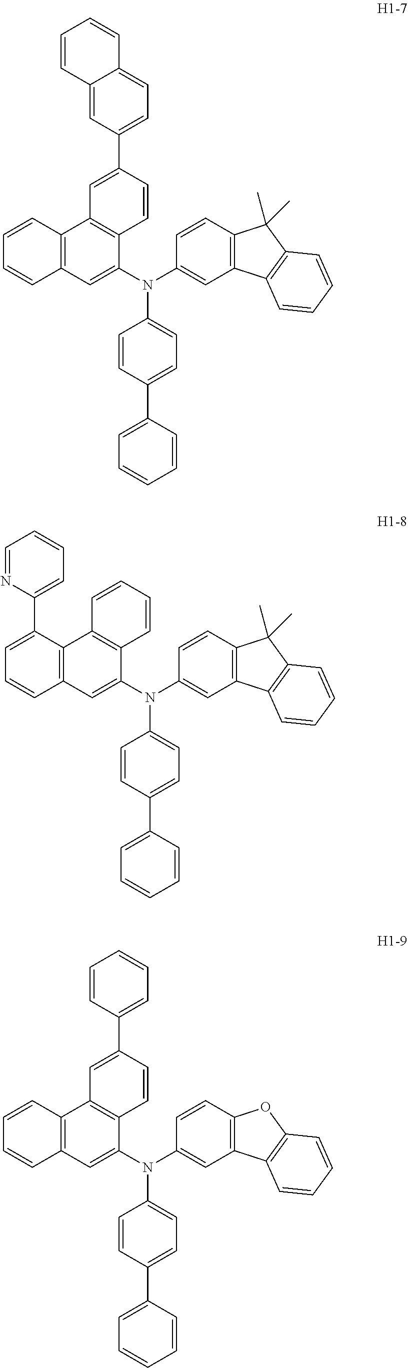 [SMILES: CC1(C)c2ccccc2-c2cc(N(c3ccc(-c4ccccc4)cc3)c3cc4ccccc4c4cc(-c5ccc6ccccc6c5)ccc34)ccc21 ; CC1(C)c2ccccc2-c2cc(N(c3ccc(-c4ccccc4)cc3)c3cc4cccc(-c5ccccn5)c4c4ccccc34)ccc21 ; c1ccc(-c2ccc(N(c3ccc4oc5ccccc5c4c3)c3cc4ccccc4c4cc(-c5ccccc5)ccc34)cc2)cc1]

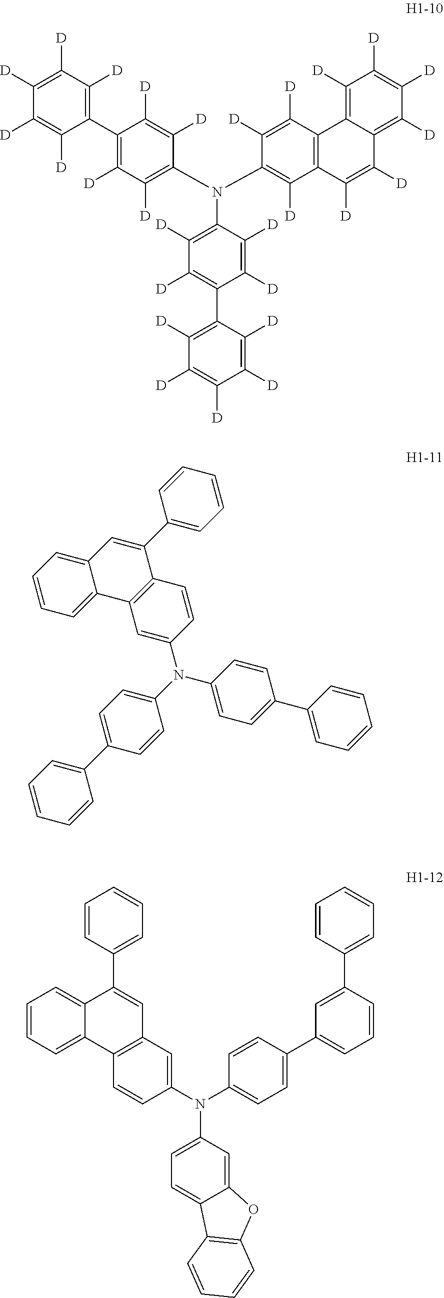 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c4c(c3[2H])c([2H])c([2H])c3c([2H])c([2H])c([2H])c([2H])c34)c([2H])c2[2H])c([2H])c1[2H] ; c1ccc(-c2ccc(N(c3ccc(-c4ccccc4)cc3)c3ccc4c(-c5ccccc5)cc5ccccc5c4c3)cc2)cc1 ; c1ccc(-c2cccc(-c3ccc(N(c4ccc5c(c4)cc(-c4ccccc4)c4ccccc45)c4ccc5c(c4)oc4ccccc45)cc3)c2)cc1]

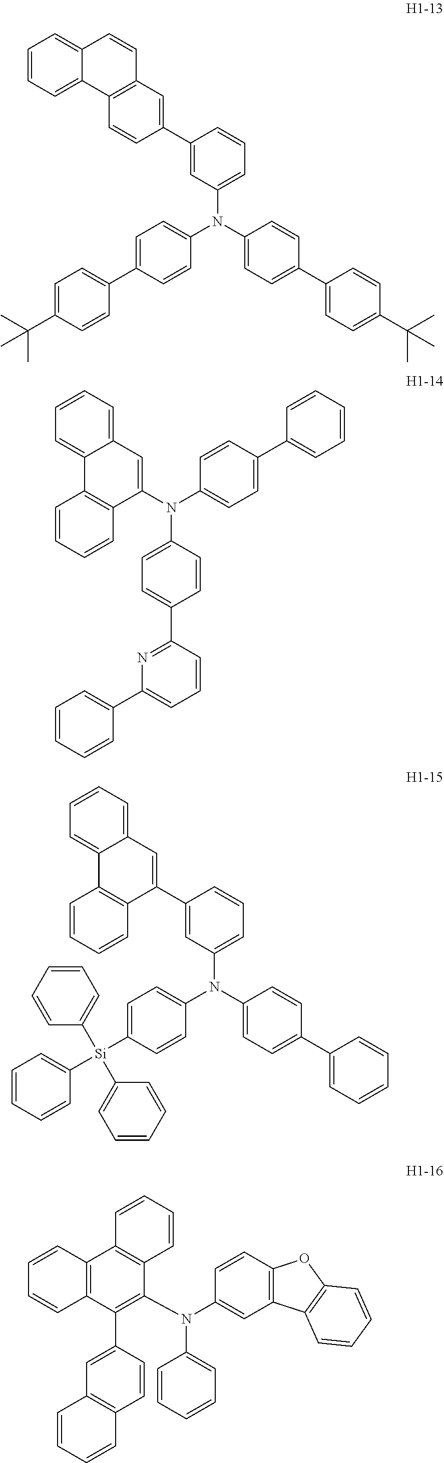

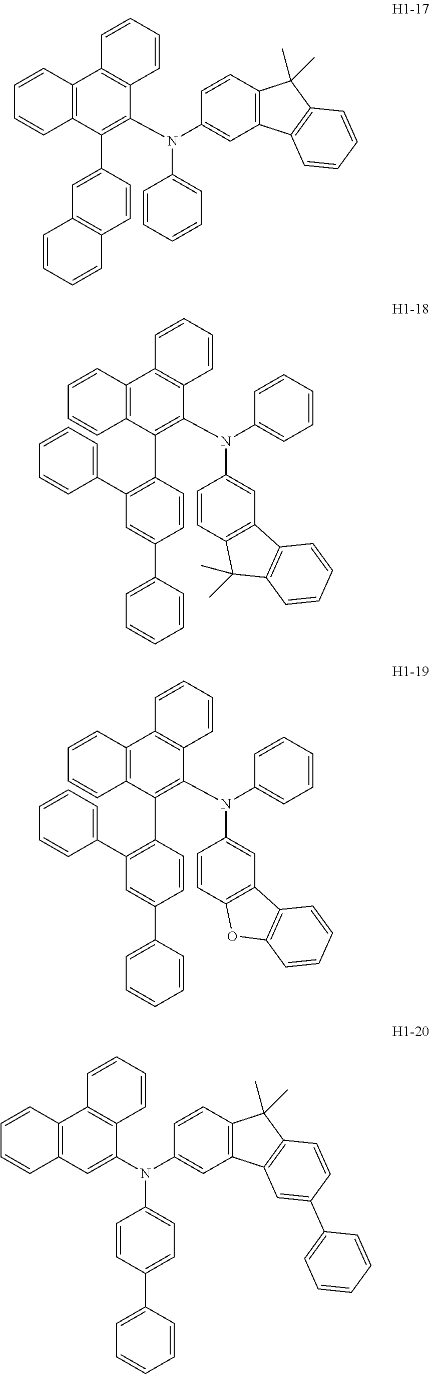 [SMILES: CC1(C)c2ccccc2-c2cc(N(c3ccccc3)c3c(-c4ccc5ccccc5c4)c4ccccc4c4ccccc34)ccc21 ; CC1(C)c2ccccc2-c2cc(N(c3ccccc3)c3c(-c4ccc(-c5ccccc5)cc4-c4ccccc4)c4ccccc4c4ccccc34)ccc21 ; c1ccc(-c2ccc(-c3c(N(c4ccccc4)c4ccc5oc6ccccc6c5c4)c4ccccc4c4ccccc34)c(-c3ccccc3)c2)cc1 ; CC1(C)c2ccc(-c3ccccc3)cc2-c2cc(N(c3ccc(-c4ccccc4)cc3)c3cc4ccccc4c4ccccc34)ccc21]

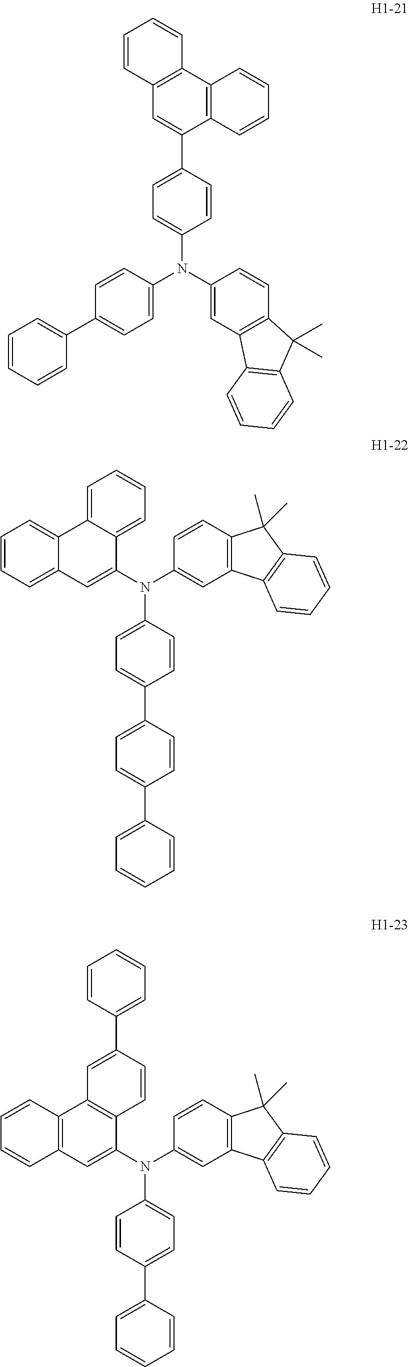 [SMILES: CC1(C)c2ccccc2-c2cc(N(c3ccc(-c4ccccc4)cc3)c3ccc(-c4cc5ccccc5c5ccccc45)cc3)ccc21 ; CC1(C)c2ccccc2-c2cc(N(c3ccc(-c4ccc(-c5ccccc5)cc4)cc3)c3cc4ccccc4c4ccccc34)ccc21 ; CC1(C)c2ccccc2-c2cc(N(c3ccc(-c4ccccc4)cc3)c3cc4ccccc4c4cc(-c5ccccc5)ccc34)ccc21]

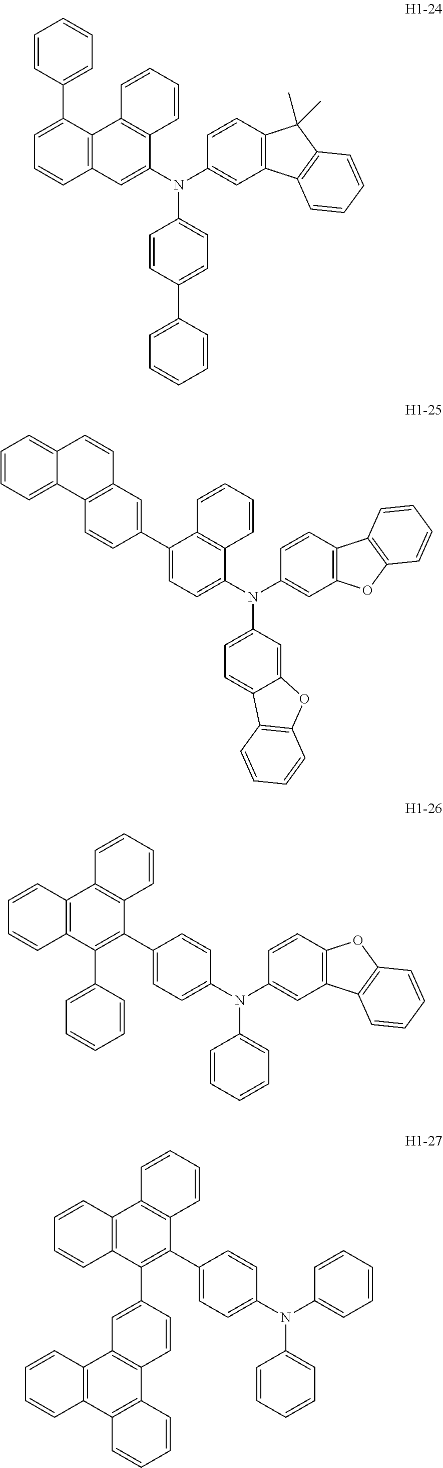 [SMILES: CC1(C)c2ccccc2-c2cc(N(c3ccc(-c4ccccc4)cc3)c3cc4cccc(-c5ccccc5)c4c4ccccc34)ccc21 ; c1ccc2c(c1)ccc1cc(-c3ccc(N(c4ccc5c(c4)oc4ccccc45)c4ccc5c(c4)oc4ccccc45)c4ccccc34)ccc12 ; c1ccc(-c2c(-c3ccc(N(c4ccccc4)c4ccc5oc6ccccc6c5c4)cc3)c3ccccc3c3ccccc23)cc1 ; c1ccc(N(c2ccccc2)c2ccc(-c3c(-c4ccc5c6ccccc6c6ccccc6c5c4)c4ccccc4c4ccccc34)cc2)cc1]

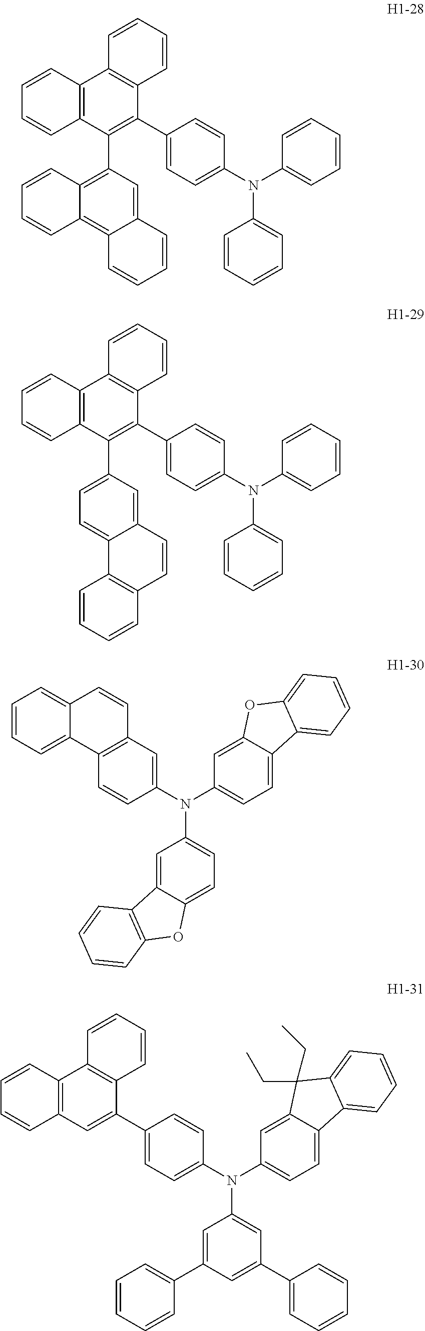

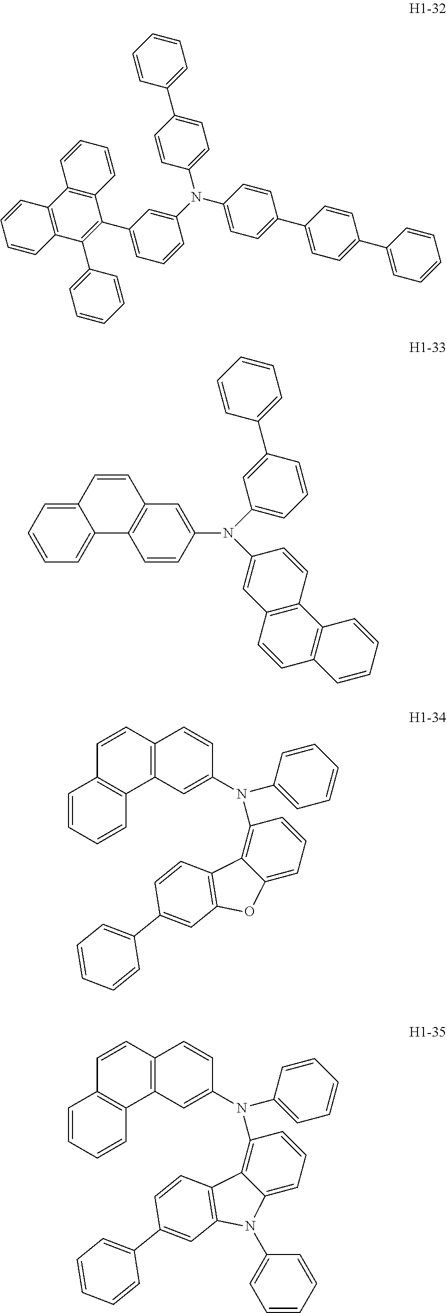 [SMILES: c1ccc(-c2ccc(-c3ccc(N(c4ccc(-c5ccccc5)cc4)c4cccc(-c5c(-c6ccccc6)c6ccccc6c6ccccc56)c4)cc3)cc2)cc1 ; c1ccc(-c2cccc(N(c3ccc4c(ccc5ccccc54)c3)c3ccc4c(ccc5ccccc54)c3)c2)cc1 ; c1ccc(-c2ccc3c(c2)oc2cccc(N(c4ccccc4)c4ccc5ccc6ccccc6c5c4)c23)cc1 ; c1ccc(-c2ccc3c4c(N(c5ccccc5)c5ccc6ccc7ccccc7c6c5)cccc4n(-c4ccccc4)c3c2)cc1]

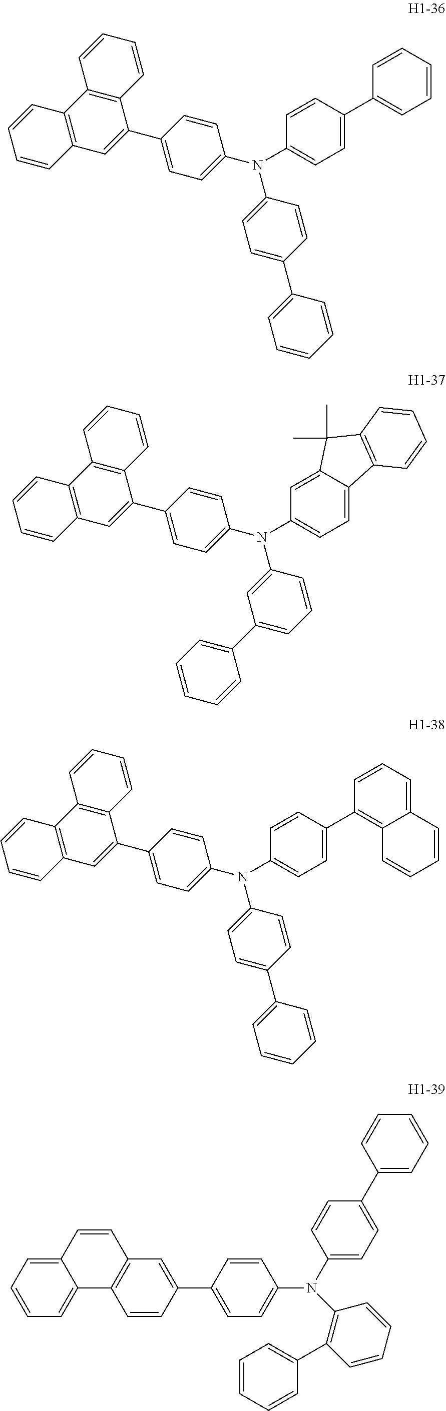 [SMILES: c1ccc(-c2ccc(N(c3ccc(-c4ccccc4)cc3)c3ccc(-c4cc5ccccc5c5ccccc45)cc3)cc2)cc1 ; CC1(C)c2ccccc2-c2ccc(N(c3ccc(-c4cc5ccccc5c5ccccc45)cc3)c3cccc(-c4ccccc4)c3)cc21 ; c1ccc(-c2ccc(N(c3ccc(-c4cccc5ccccc45)cc3)c3ccc(-c4cc5ccccc5c5ccccc45)cc3)cc2)cc1 ; c1ccc(-c2ccc(N(c3ccc(-c4ccc5c(ccc6ccccc65)c4)cc3)c3ccccc3-c3ccccc3)cc2)cc1]

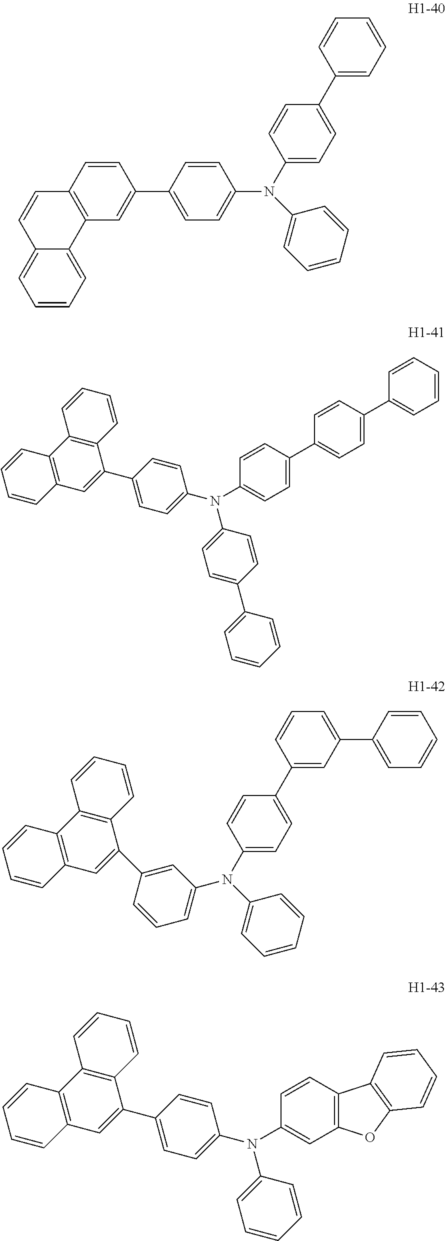 [SMILES: c1ccc(-c2ccc(N(c3ccccc3)c3ccc(-c4ccc5ccc6ccccc6c5c4)cc3)cc2)cc1 ; c1ccc(-c2ccc(-c3ccc(N(c4ccc(-c5ccccc5)cc4)c4ccc(-c5cc6ccccc6c6ccccc56)cc4)cc3)cc2)cc1 ; c1ccc(-c2cccc(-c3ccc(N(c4ccccc4)c4cccc(-c5cc6ccccc6c6ccccc56)c4)cc3)c2)cc1 ; c1ccc(N(c2ccc(-c3cc4ccccc4c4ccccc34)cc2)c2ccc3c(c2)oc2ccccc23)cc1]

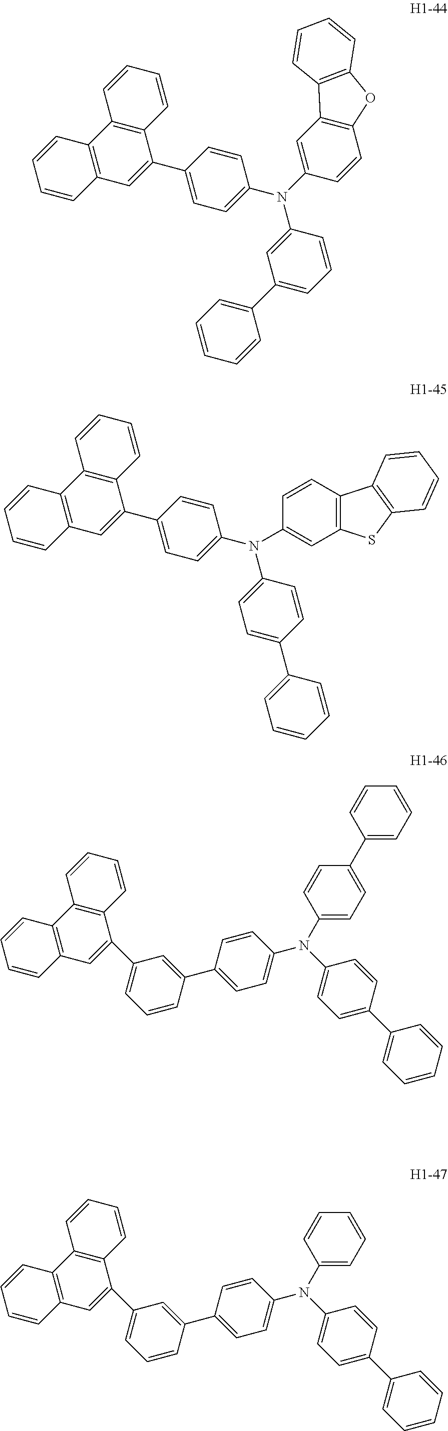 [SMILES: c1ccc(-c2cccc(N(c3ccc(-c4cc5ccccc5c5ccccc45)cc3)c3ccc4oc5ccccc5c4c3)c2)cc1 ; c1ccc(-c2ccc(N(c3ccc(-c4cc5ccccc5c5ccccc45)cc3)c3ccc4c(c3)sc3ccccc34)cc2)cc1 ; c1ccc(-c2ccc(N(c3ccc(-c4ccccc4)cc3)c3ccc(-c4cccc(-c5cc6ccccc6c6ccccc56)c4)cc3)cc2)cc1 ; c1ccc(-c2ccc(N(c3ccccc3)c3ccc(-c4cccc(-c5cc6ccccc6c6ccccc56)c4)cc3)cc2)cc1]

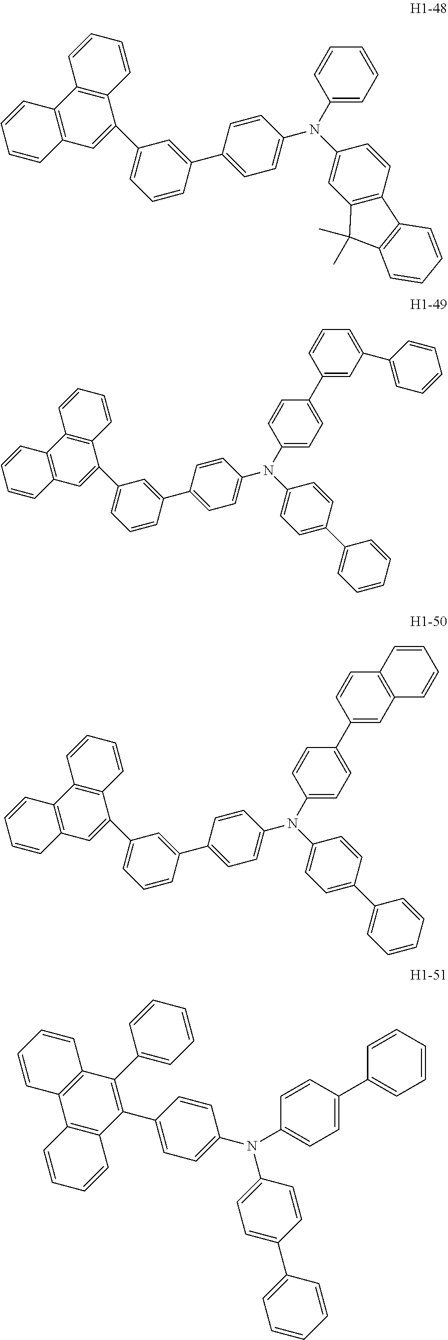 [SMILES: CC1(C)c2ccccc2-c2ccc(N(c3ccccc3)c3ccc(-c4cccc(-c5cc6ccccc6c6ccccc56)c4)cc3)cc21 ; c1ccc(-c2ccc(N(c3ccc(-c4cccc(-c5ccccc5)c4)cc3)c3ccc(-c4cccc(-c5cc6ccccc6c6ccccc56)c4)cc3)cc2)cc1 ; c1ccc(-c2ccc(N(c3ccc(-c4cccc(-c5cc6ccccc6c6ccccc56)c4)cc3)c3ccc(-c4ccc5ccccc5c4)cc3)cc2)cc1 ; c1ccc(-c2ccc(N(c3ccc(-c4ccccc4)cc3)c3ccc(-c4c(-c5ccccc5)c5ccccc5c5ccccc45)cc3)cc2)cc1]

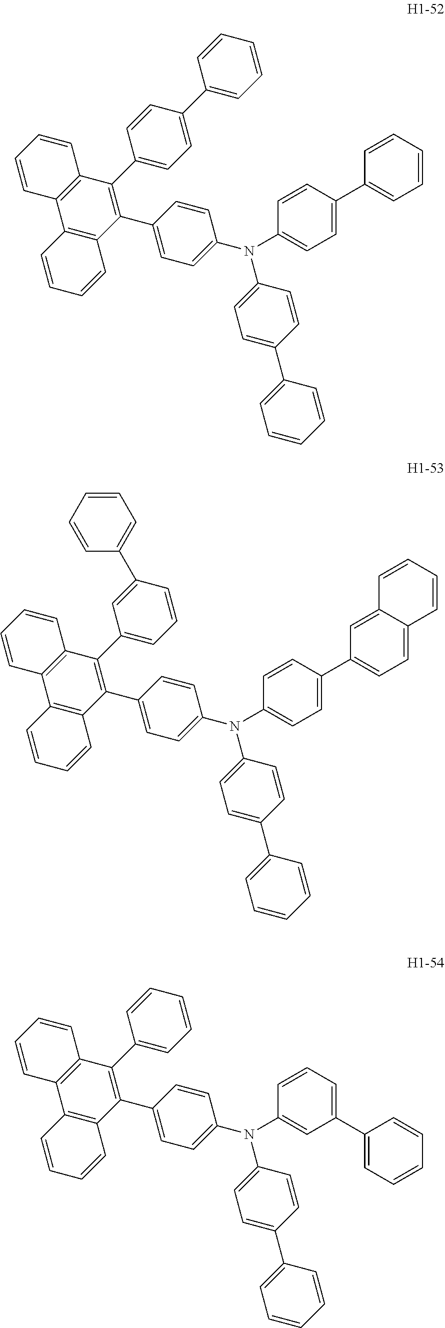 [SMILES: c1ccc(-c2ccc(-c3c(-c4ccc(N(c5ccc(-c6ccccc6)cc5)c5ccc(-c6ccccc6)cc5)cc4)c4ccccc4c4ccccc34)cc2)cc1 ; c1ccc(-c2ccc(N(c3ccc(-c4ccc5ccccc5c4)cc3)c3ccc(-c4c(-c5cccc(-c6ccccc6)c5)c5ccccc5c5ccccc45)cc3)cc2)cc1 ; c1ccc(-c2ccc(N(c3ccc(-c4c(-c5ccccc5)c5ccccc5c5ccccc45)cc3)c3cccc(-c4ccccc4)c3)cc2)cc1]

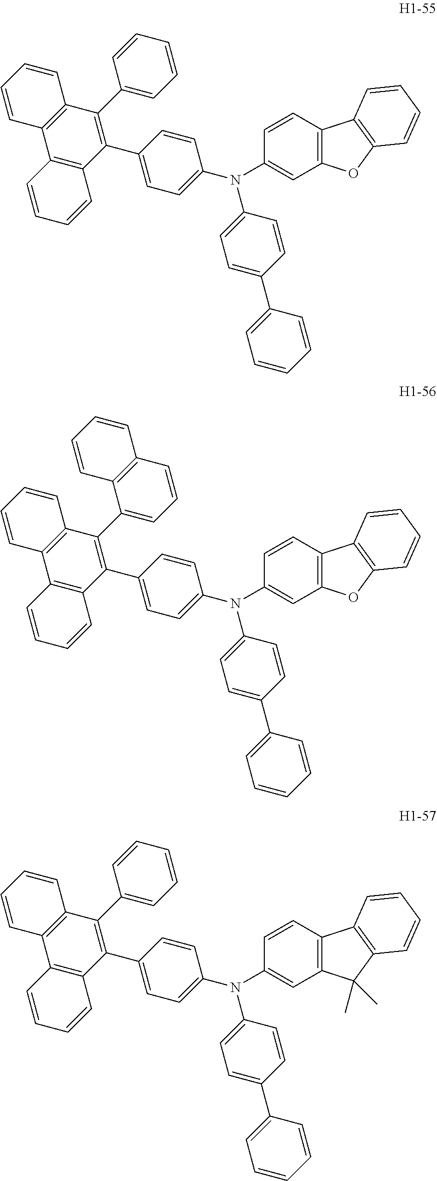 [SMILES: c1ccc(-c2ccc(N(c3ccc(-c4c(-c5ccccc5)c5ccccc5c5ccccc45)cc3)c3ccc4c(c3)oc3ccccc34)cc2)cc1 ; c1ccc(-c2ccc(N(c3ccc(-c4c(-c5cccc6ccccc56)c5ccccc5c5ccccc45)cc3)c3ccc4c(c3)oc3ccccc34)cc2)cc1 ; CC1(C)c2ccccc2-c2ccc(N(c3ccc(-c4ccccc4)cc3)c3ccc(-c4c(-c5ccccc5)c5ccccc5c5ccccc45)cc3)cc21]

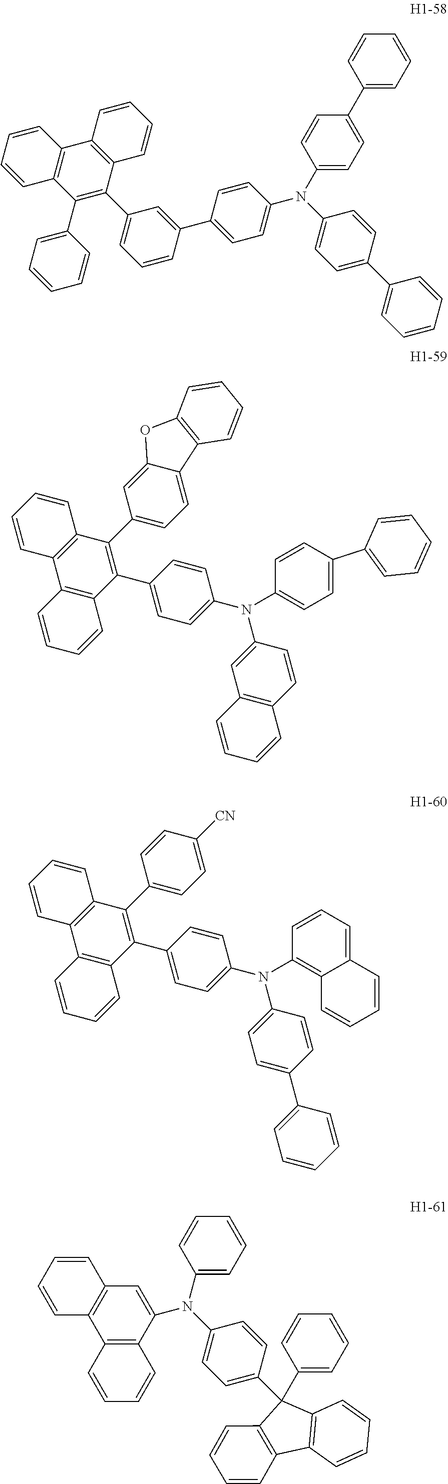 [SMILES: c1ccc(-c2ccc(N(c3ccc(-c4ccccc4)cc3)c3ccc(-c4cccc(-c5c(-c6ccccc6)c6ccccc6c6ccccc56)c4)cc3)cc2)cc1 ; c1ccc(-c2ccc(N(c3ccc(-c4c(-c5ccc6c(c5)oc5ccccc56)c5ccccc5c5ccccc45)cc3)c3ccc4ccccc4c3)cc2)cc1 ; N#Cc1ccc(-c2c(-c3ccc(N(c4ccc(-c5ccccc5)cc4)c4cccc5ccccc45)cc3)c3ccccc3c3ccccc23)cc1 ; c1ccc(N(c2ccc(C3(c4ccccc4)c4ccccc4-c4ccccc43)cc2)c2cc3ccccc3c3ccccc23)cc1]

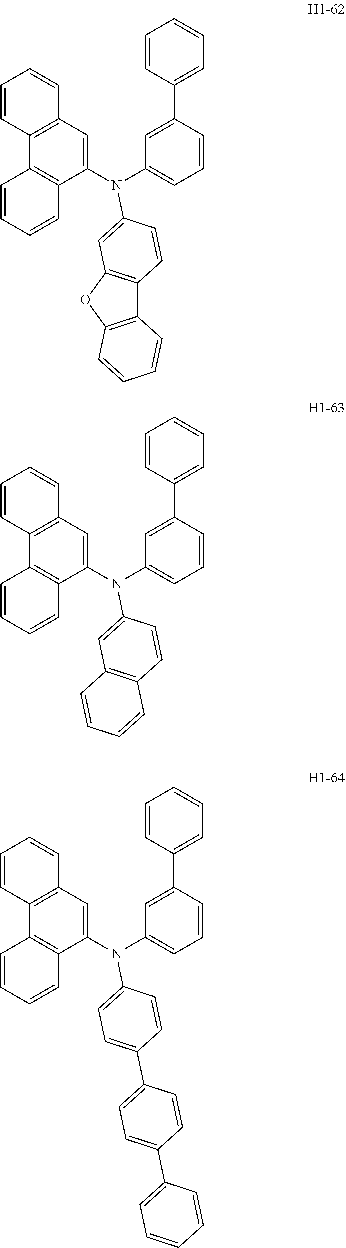 [SMILES: c1ccc(-c2cccc(N(c3ccc4c(c3)oc3ccccc34)c3cc4ccccc4c4ccccc34)c2)cc1 ; c1ccc(-c2cccc(N(c3ccc4ccccc4c3)c3cc4ccccc4c4ccccc34)c2)cc1 ; c1ccc(-c2ccc(-c3ccc(N(c4cccc(-c5ccccc5)c4)c4cc5ccccc5c5ccccc45)cc3)cc2)cc1]

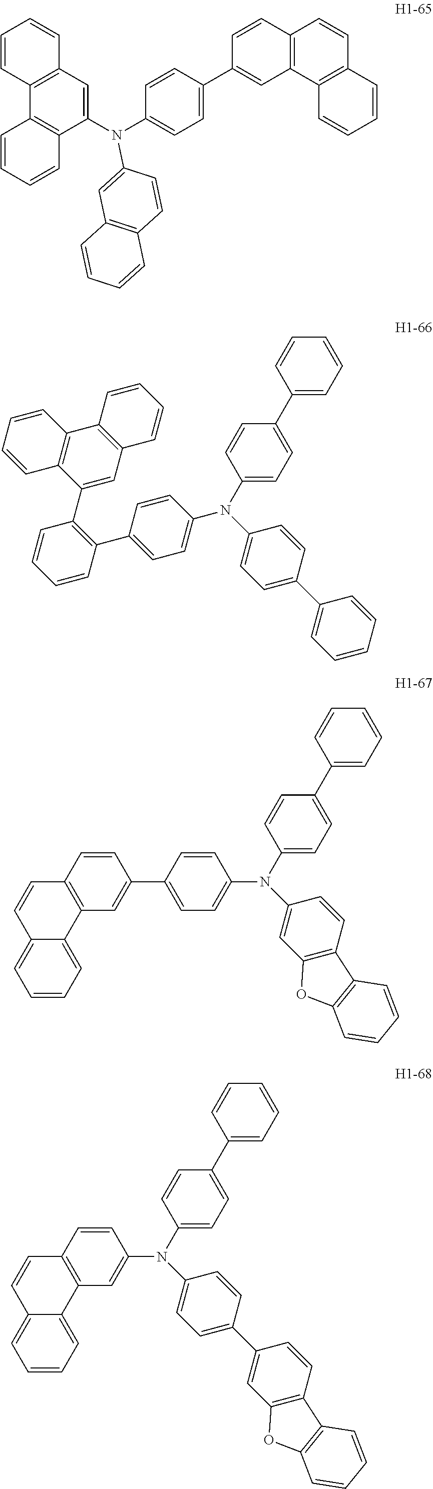 [SMILES: c1ccc2cc(N(c3ccc(-c4ccc5ccc6ccccc6c5c4)cc3)c3cc4ccccc4c4ccccc34)ccc2c1 ; c1ccc(-c2ccc(N(c3ccc(-c4ccccc4)cc3)c3ccc(-c4ccccc4-c4cc5ccccc5c5ccccc45)cc3)cc2)cc1 ; c1ccc(-c2ccc(N(c3ccc(-c4ccc5ccc6ccccc6c5c4)cc3)c3ccc4c(c3)oc3ccccc34)cc2)cc1 ; c1ccc(-c2ccc(N(c3ccc(-c4ccc5c(c4)oc4ccccc45)cc3)c3ccc4ccc5ccccc5c4c3)cc2)cc1]

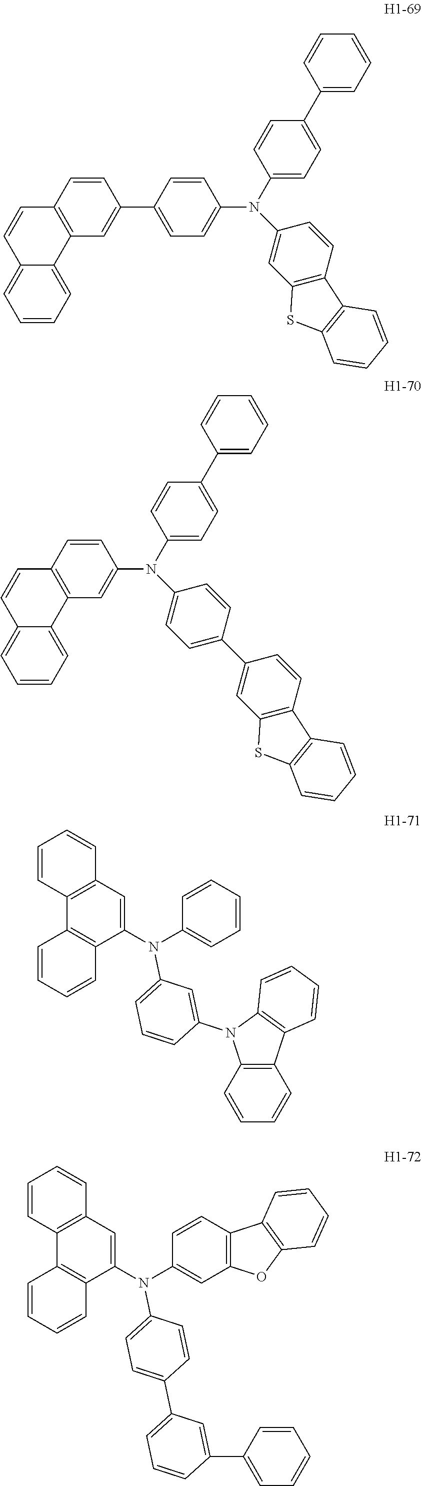 [SMILES: c1ccc(-c2ccc(N(c3ccc(-c4ccc5ccc6ccccc6c5c4)cc3)c3ccc4c(c3)sc3ccccc34)cc2)cc1 ; c1ccc(-c2ccc(N(c3ccc(-c4ccc5c(c4)sc4ccccc45)cc3)c3ccc4ccc5ccccc5c4c3)cc2)cc1 ; c1ccc(N(c2cccc(-n3c4ccccc4c4ccccc43)c2)c2cc3ccccc3c3ccccc23)cc1 ; c1ccc(-c2cccc(-c3ccc(N(c4ccc5c(c4)oc4ccccc45)c4cc5ccccc5c5ccccc45)cc3)c2)cc1]

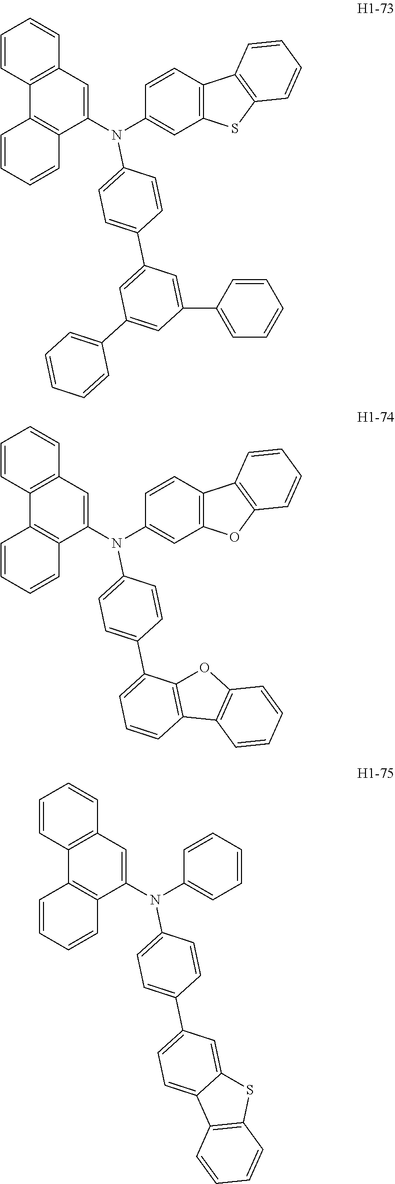

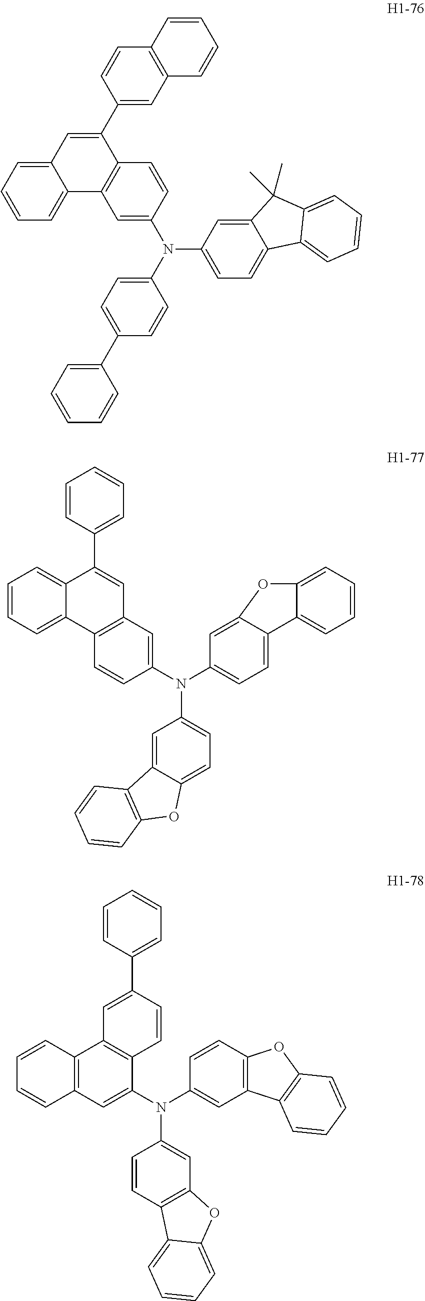

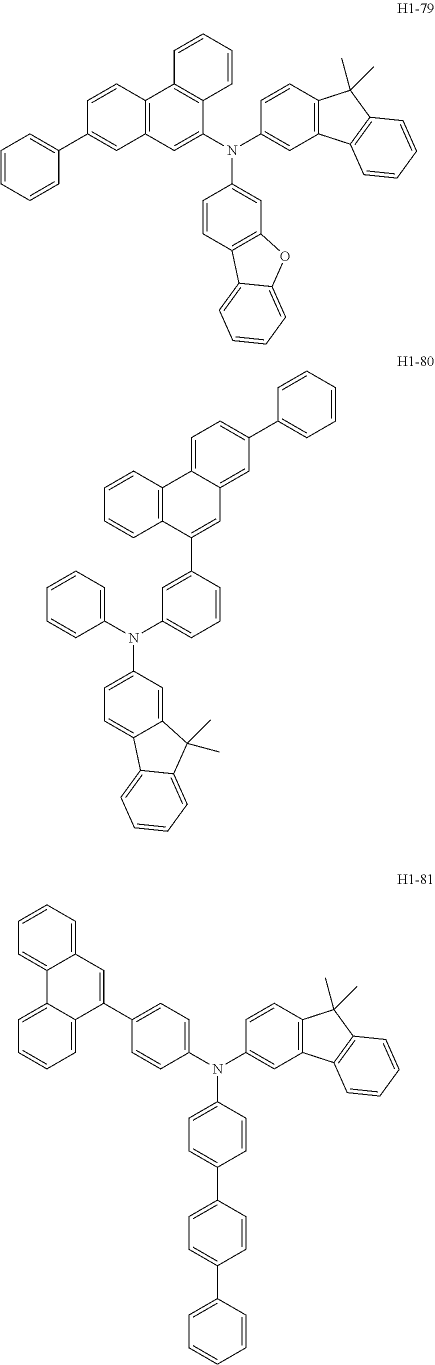

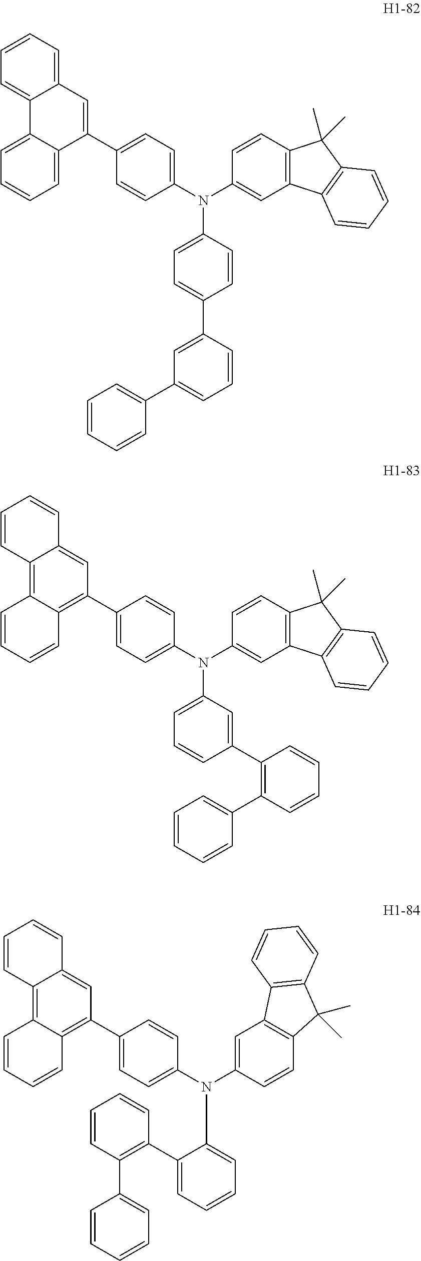 [SMILES: CC1(C)c2ccccc2-c2cc(N(c3ccc(-c4cccc(-c5ccccc5)c4)cc3)c3ccc(-c4cc5ccccc5c5ccccc45)cc3)ccc21 ; CC1(C)c2ccccc2-c2cc(N(c3ccc(-c4cc5ccccc5c5ccccc45)cc3)c3cccc(-c4ccccc4-c4ccccc4)c3)ccc21 ; CC1(C)c2ccccc2-c2cc(N(c3ccc(-c4cc5ccccc5c5ccccc45)cc3)c3ccccc3-c3ccccc3-c3ccccc3)ccc21]

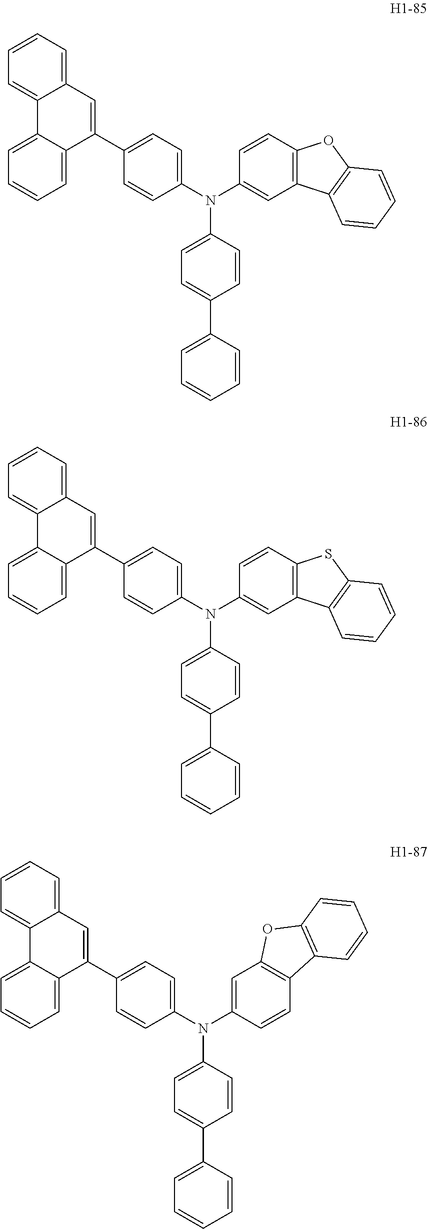 [SMILES: c1ccc(-c2ccc(N(c3ccc(-c4cc5ccccc5c5ccccc45)cc3)c3ccc4oc5ccccc5c4c3)cc2)cc1 ; c1ccc(-c2ccc(N(c3ccc(-c4cc5ccccc5c5ccccc45)cc3)c3ccc4sc5ccccc5c4c3)cc2)cc1 ; c1ccc(-c2ccc(N(c3ccc(-c4cc5ccccc5c5ccccc45)cc3)c3ccc4c(c3)oc3ccccc34)cc2)cc1]

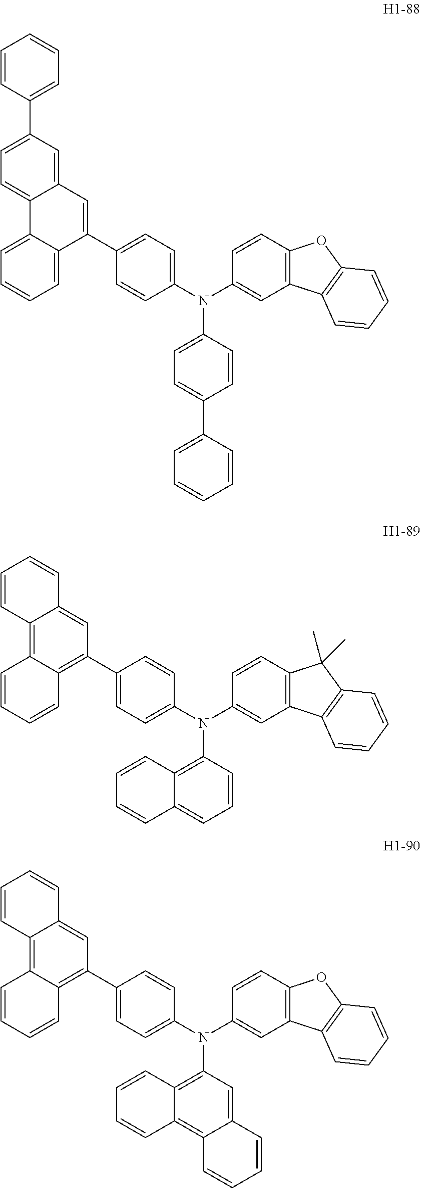 [SMILES: c1ccc(-c2ccc(N(c3ccc(-c4cc5cc(-c6ccccc6)ccc5c5ccccc45)cc3)c3ccc4oc5ccccc5c4c3)cc2)cc1 ; CC1(C)c2ccccc2-c2cc(N(c3ccc(-c4cc5ccccc5c5ccccc45)cc3)c3cccc4ccccc34)ccc21 ; c1ccc2c(c1)cc(-c1ccc(N(c3ccc4oc5ccccc5c4c3)c3cc4ccccc4c4ccccc34)cc1)c1ccccc12]

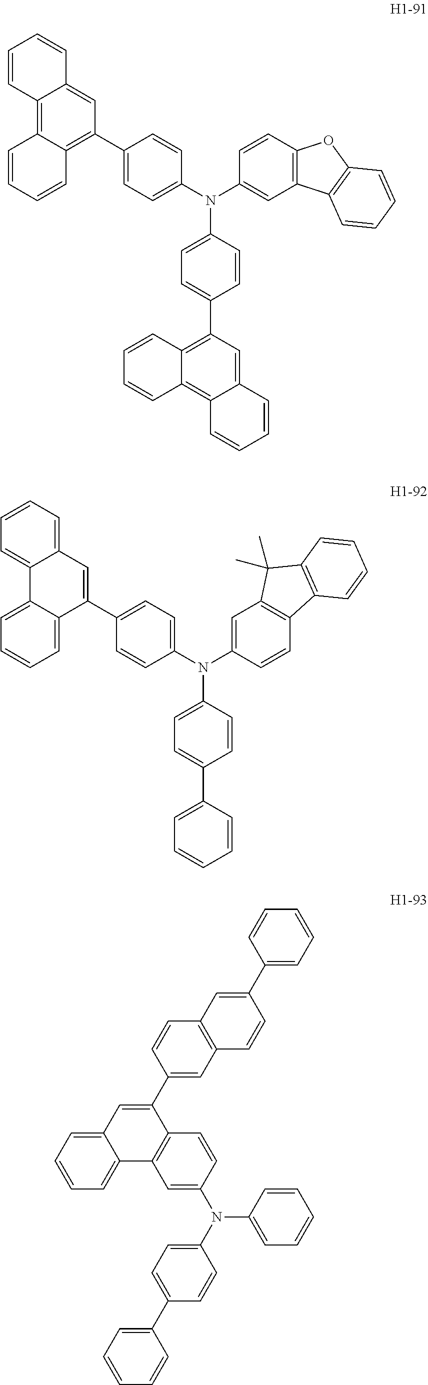 [SMILES: c1ccc2c(c1)cc(-c1ccc(N(c3ccc(-c4cc5ccccc5c5ccccc45)cc3)c3ccc4oc5ccccc5c4c3)cc1)c1ccccc12 ; CC1(C)c2ccccc2-c2ccc(N(c3ccc(-c4ccccc4)cc3)c3ccc(-c4cc5ccccc5c5ccccc45)cc3)cc21 ; c1ccc(-c2ccc(N(c3ccccc3)c3ccc4c(-c5ccc6cc(-c7ccccc7)ccc6c5)cc5ccccc5c4c3)cc2)cc1]

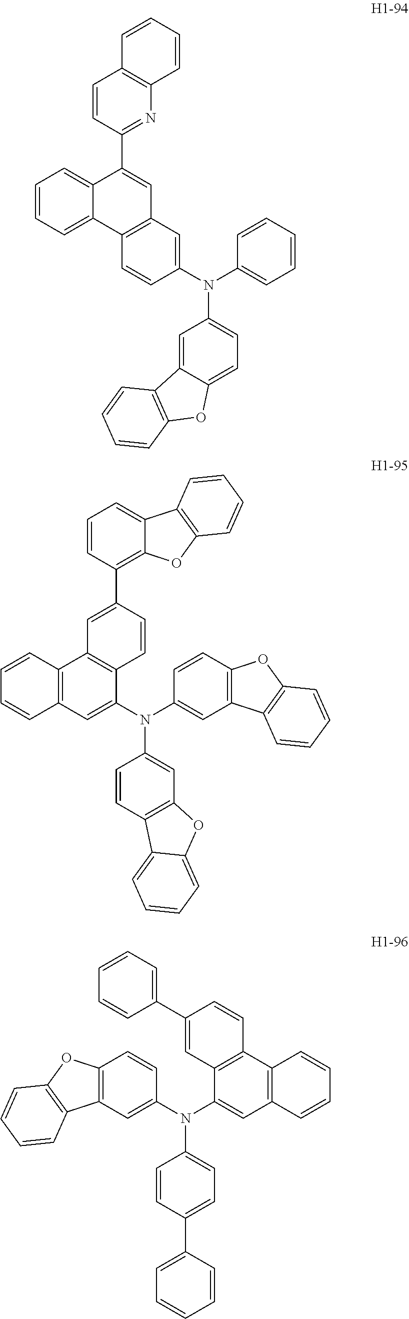

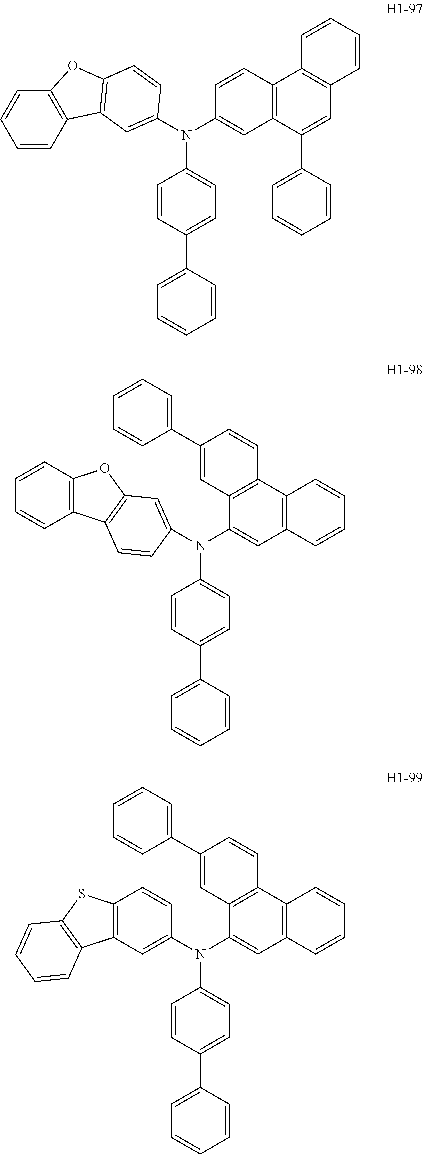 [SMILES: c1ccc(-c2ccc(N(c3ccc4c(c3)c(-c3ccccc3)cc3ccccc34)c3ccc4oc5ccccc5c4c3)cc2)cc1 ; c1ccc(-c2ccc(N(c3ccc4c(c3)oc3ccccc34)c3cc4ccccc4c4ccc(-c5ccccc5)cc34)cc2)cc1 ; c1ccc(-c2ccc(N(c3ccc4sc5ccccc5c4c3)c3cc4ccccc4c4ccc(-c5ccccc5)cc34)cc2)cc1]

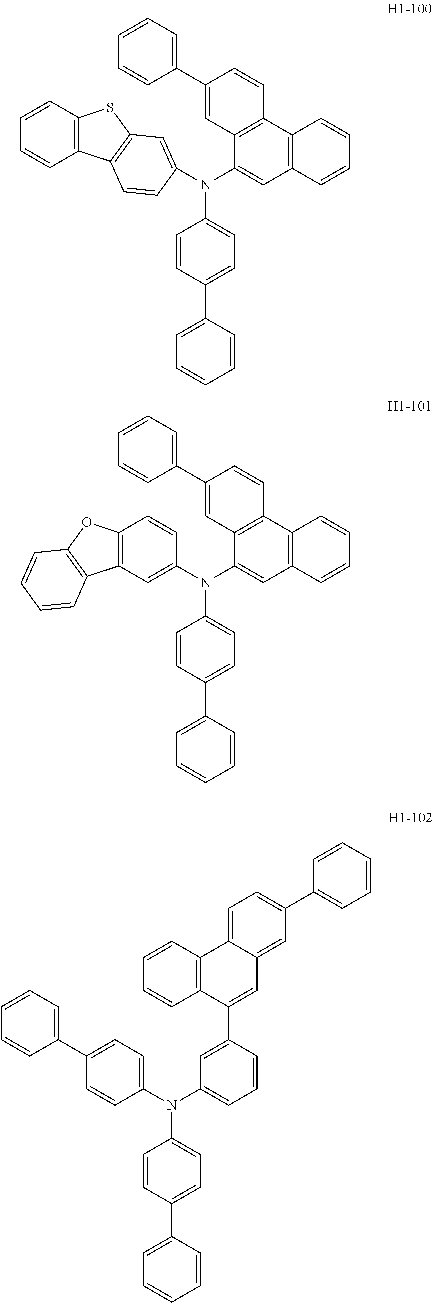 [SMILES: c1ccc(-c2ccc(N(c3ccc4c(c3)sc3ccccc34)c3cc4ccccc4c4ccc(-c5ccccc5)cc34)cc2)cc1 ; c1ccc(-c2ccc(N(c3ccc4oc5ccccc5c4c3)c3cc4ccccc4c4ccc(-c5ccccc5)cc34)cc2)cc1 ; c1ccc(-c2ccc(N(c3ccc(-c4ccccc4)cc3)c3cccc(-c4cc5cc(-c6ccccc6)ccc5c5ccccc45)c3)cc2)cc1]

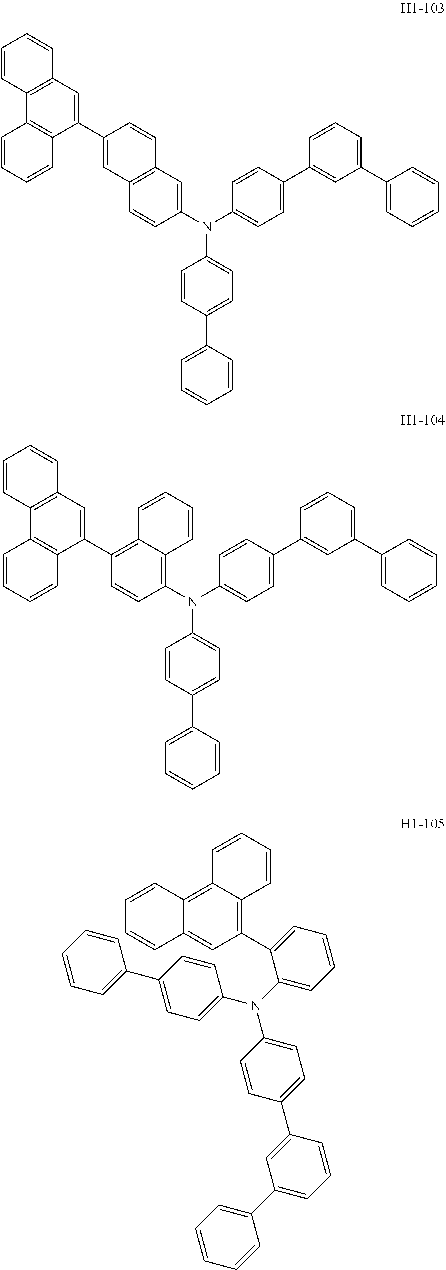 [SMILES: c1ccc(-c2ccc(N(c3ccc(-c4cccc(-c5ccccc5)c4)cc3)c3ccc4cc(-c5cc6ccccc6c6ccccc56)ccc4c3)cc2)cc1 ; c1ccc(-c2ccc(N(c3ccc(-c4cccc(-c5ccccc5)c4)cc3)c3ccc(-c4cc5ccccc5c5ccccc45)c4ccccc34)cc2)cc1 ; c1ccc(-c2ccc(N(c3ccc(-c4cccc(-c5ccccc5)c4)cc3)c3ccccc3-c3cc4ccccc4c4ccccc34)cc2)cc1]

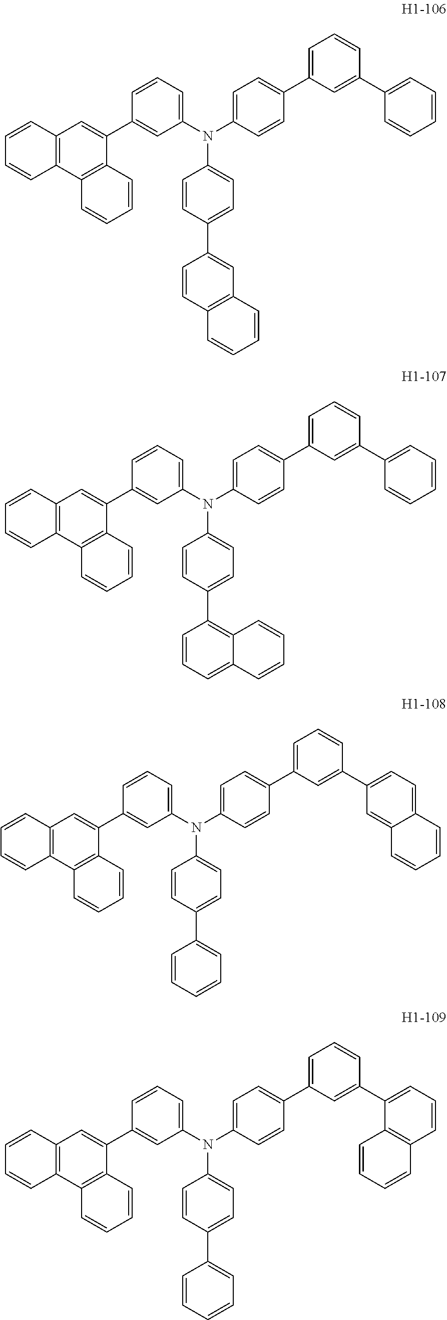 [SMILES: c1ccc(-c2cccc(-c3ccc(N(c4ccc(-c5ccc6ccccc6c5)cc4)c4cccc(-c5cc6ccccc6c6ccccc56)c4)cc3)c2)cc1 ; c1ccc(-c2cccc(-c3ccc(N(c4ccc(-c5cccc6ccccc56)cc4)c4cccc(-c5cc6ccccc6c6ccccc56)c4)cc3)c2)cc1 ; c1ccc(-c2ccc(N(c3ccc(-c4cccc(-c5ccc6ccccc6c5)c4)cc3)c3cccc(-c4cc5ccccc5c5ccccc45)c3)cc2)cc1 ; c1ccc(-c2ccc(N(c3ccc(-c4cccc(-c5cccc6ccccc56)c4)cc3)c3cccc(-c4cc5ccccc5c5ccccc45)c3)cc2)cc1]

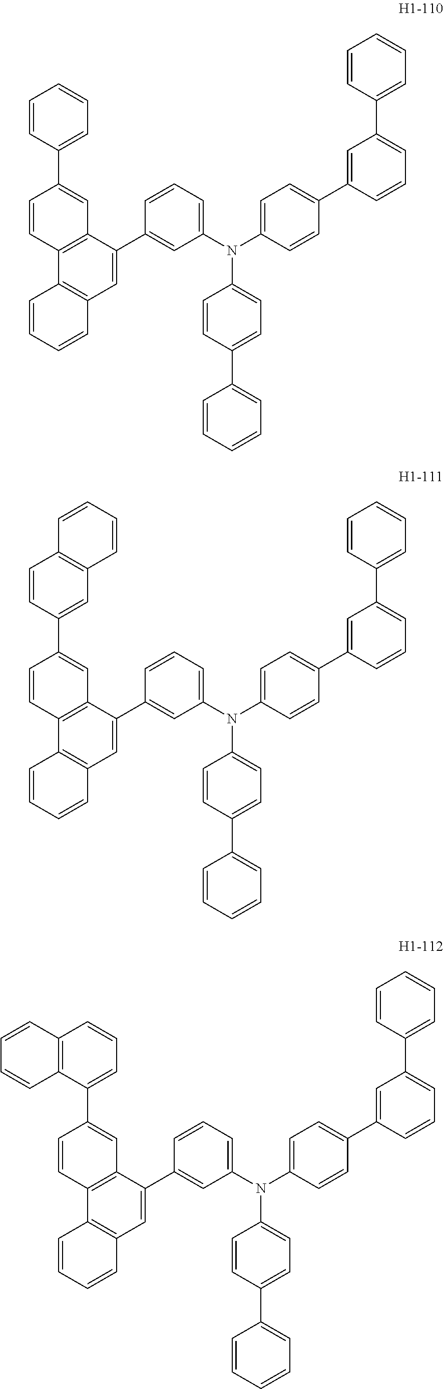 [SMILES: c1ccc(-c2ccc(N(c3ccc(-c4cccc(-c5ccccc5)c4)cc3)c3cccc(-c4cc5ccccc5c5ccc(-c6ccccc6)cc45)c3)cc2)cc1 ; c1ccc(-c2ccc(N(c3ccc(-c4cccc(-c5ccccc5)c4)cc3)c3cccc(-c4cc5ccccc5c5ccc(-c6ccc7ccccc7c6)cc45)c3)cc2)cc1 ; c1ccc(-c2ccc(N(c3ccc(-c4cccc(-c5ccccc5)c4)cc3)c3cccc(-c4cc5ccccc5c5ccc(-c6cccc7ccccc67)cc45)c3)cc2)cc1]

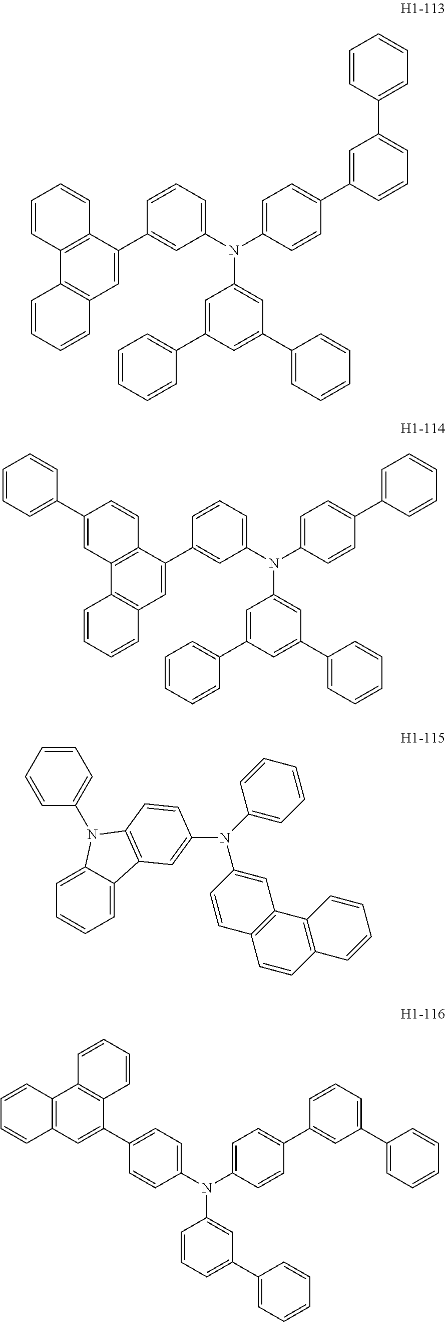 [SMILES: c1ccc(-c2cccc(-c3ccc(N(c4cc(-c5ccccc5)cc(-c5ccccc5)c4)c4cccc(-c5cc6ccccc6c6ccccc56)c4)cc3)c2)cc1 ; c1ccc(-c2ccc(N(c3cc(-c4ccccc4)cc(-c4ccccc4)c3)c3cccc(-c4cc5ccccc5c5cc(-c6ccccc6)ccc45)c3)cc2)cc1 ; c1ccc(N(c2ccc3ccc4ccccc4c3c2)c2ccc3c(c2)c2ccccc2n3-c2ccccc2)cc1 ; c1ccc(-c2cccc(-c3ccc(N(c4ccc(-c5cc6ccccc6c6ccccc56)cc4)c4cccc(-c5ccccc5)c4)cc3)c2)cc1]

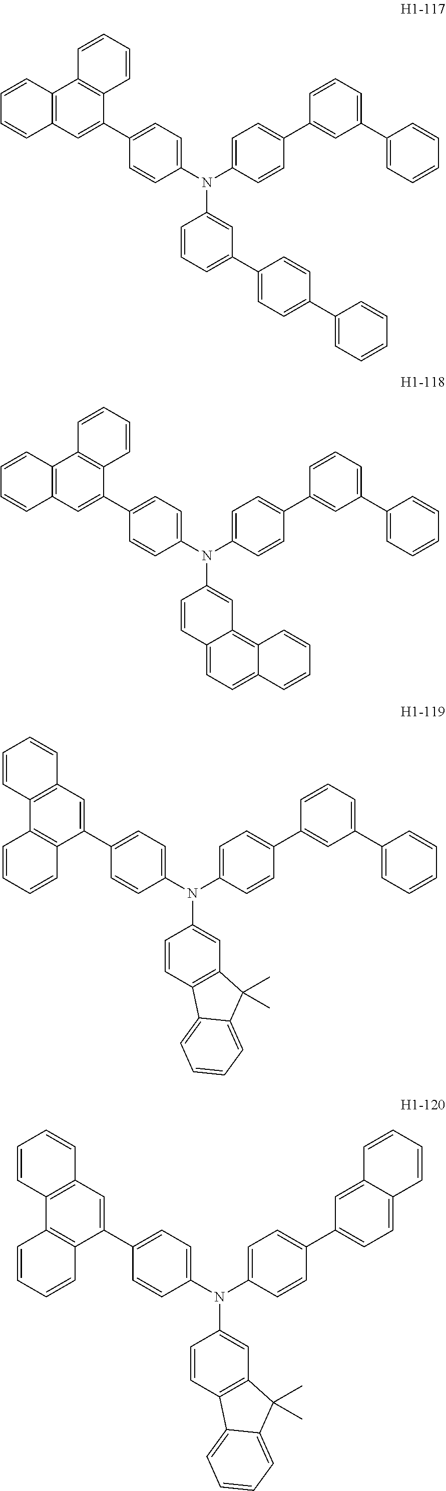 [SMILES: c1ccc(-c2ccc(-c3cccc(N(c4ccc(-c5cccc(-c6ccccc6)c5)cc4)c4ccc(-c5cc6ccccc6c6ccccc56)cc4)c3)cc2)cc1 ; c1ccc(-c2cccc(-c3ccc(N(c4ccc(-c5cc6ccccc6c6ccccc56)cc4)c4ccc5ccc6ccccc6c5c4)cc3)c2)cc1 ; CC1(C)c2ccccc2-c2ccc(N(c3ccc(-c4cccc(-c5ccccc5)c4)cc3)c3ccc(-c4cc5ccccc5c5ccccc45)cc3)cc21 ; CC1(C)c2ccccc2-c2ccc(N(c3ccc(-c4ccc5ccccc5c4)cc3)c3ccc(-c4cc5ccccc5c5ccccc45)cc3)cc21]

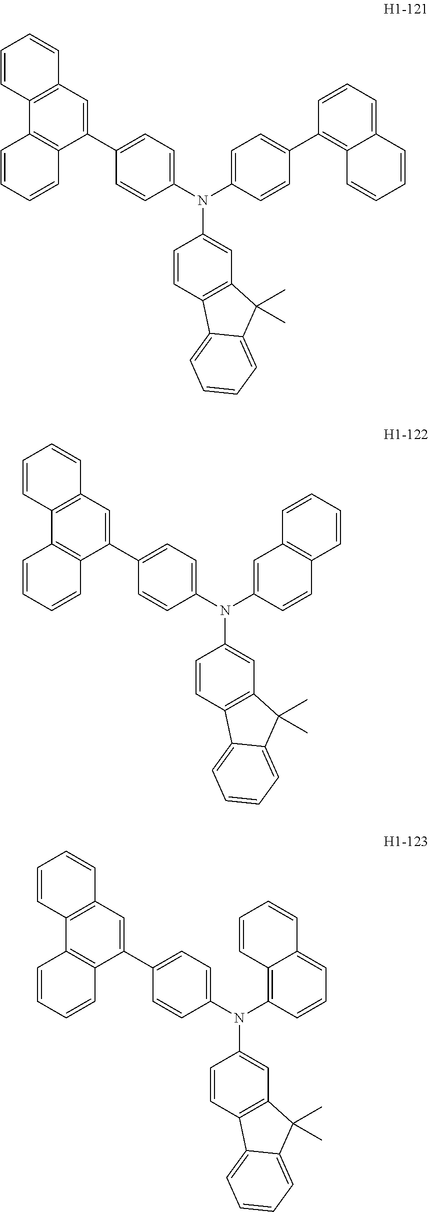 [SMILES: CC1(C)c2ccccc2-c2ccc(N(c3ccc(-c4cccc5ccccc45)cc3)c3ccc(-c4cc5ccccc5c5ccccc45)cc3)cc21 ; CC1(C)c2ccccc2-c2ccc(N(c3ccc(-c4cc5ccccc5c5ccccc45)cc3)c3ccc4ccccc4c3)cc21 ; CC1(C)c2ccccc2-c2ccc(N(c3ccc(-c4cc5ccccc5c5ccccc45)cc3)c3cccc4ccccc34)cc21]

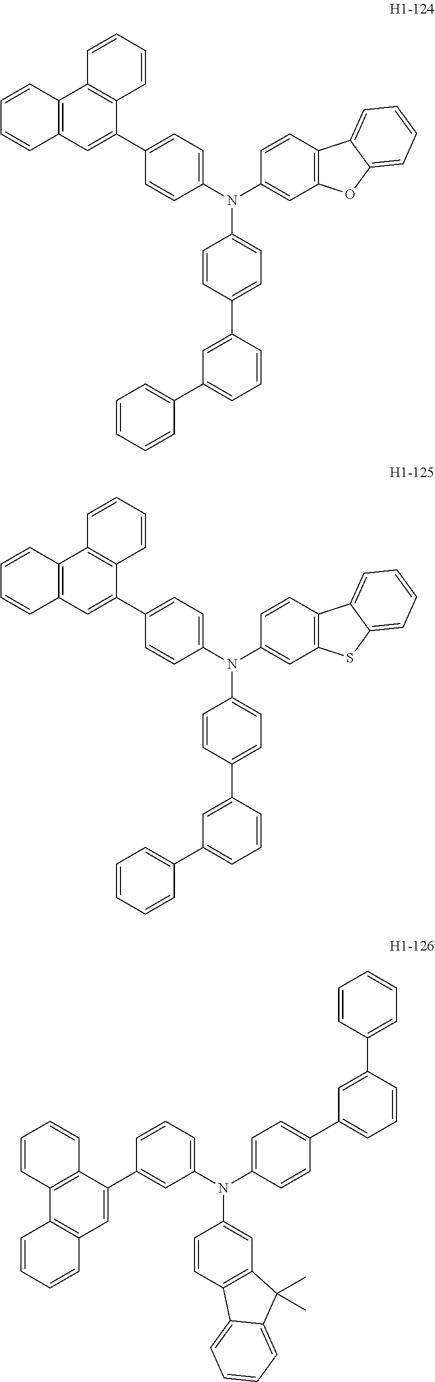 [SMILES: c1ccc(-c2cccc(-c3ccc(N(c4ccc(-c5cc6ccccc6c6ccccc56)cc4)c4ccc5c(c4)oc4ccccc45)cc3)c2)cc1 ; c1ccc(-c2cccc(-c3ccc(N(c4ccc(-c5cc6ccccc6c6ccccc56)cc4)c4ccc5c(c4)sc4ccccc45)cc3)c2)cc1 ; CC1(C)c2ccccc2-c2ccc(N(c3ccc(-c4cccc(-c5ccccc5)c4)cc3)c3cccc(-c4cc5ccccc5c5ccccc45)c3)cc21]

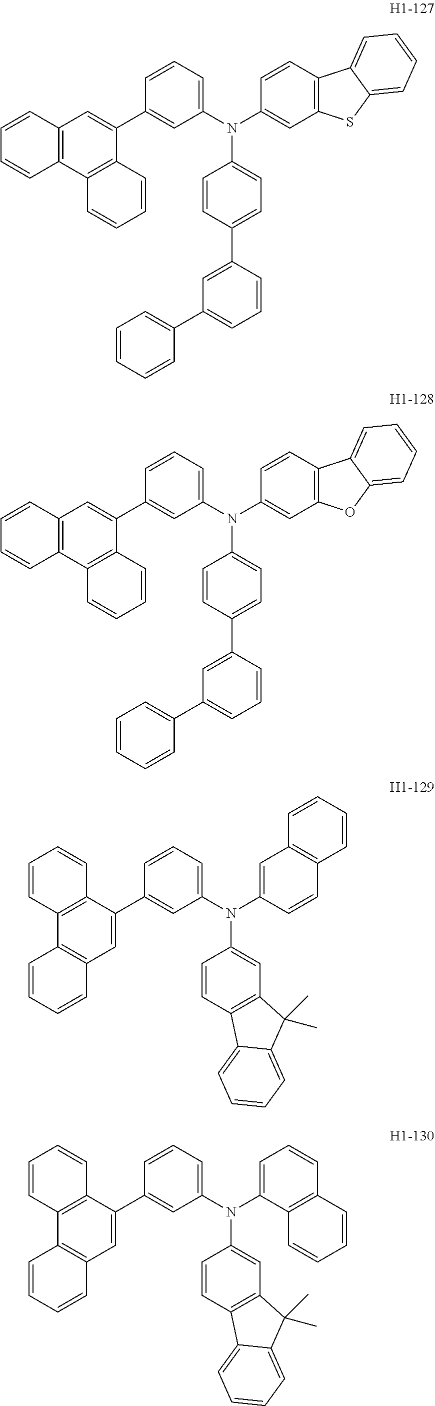

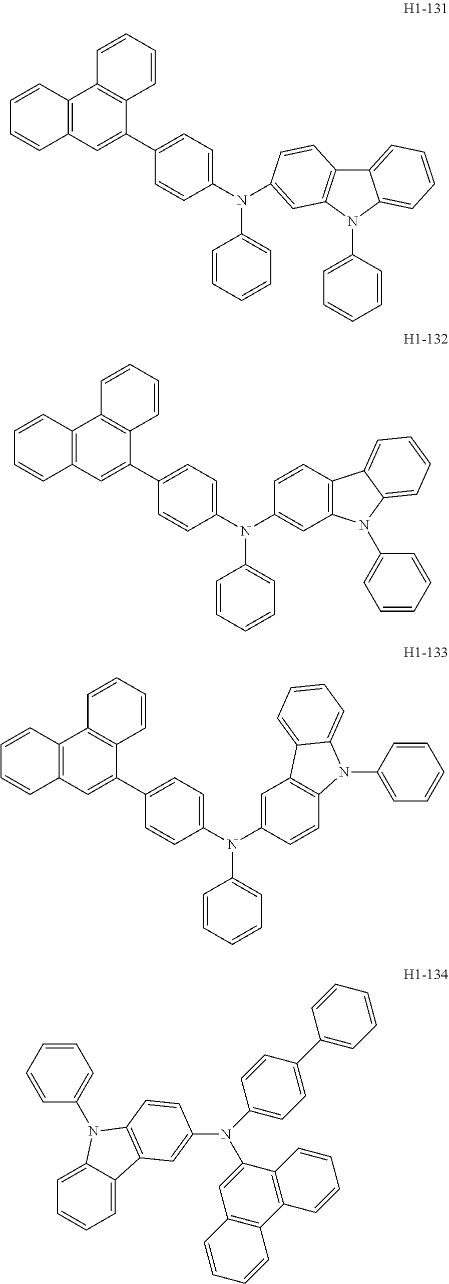 [SMILES: c1ccc(N(c2ccc(-c3cc4ccccc4c4ccccc34)cc2)c2ccc3c4ccccc4n(-c4ccccc4)c3c2)cc1 ; c1ccc(N(c2ccc(-c3cc4ccccc4c4ccccc34)cc2)c2ccc3c4ccccc4n(-c4ccccc4)c3c2)cc1 ; c1ccc(N(c2ccc(-c3cc4ccccc4c4ccccc34)cc2)c2ccc3c(c2)c2ccccc2n3-c2ccccc2)cc1 ; c1ccc(-c2ccc(N(c3ccc4c(c3)c3ccccc3n4-c3ccccc3)c3cc4ccccc4c4ccccc34)cc2)cc1]

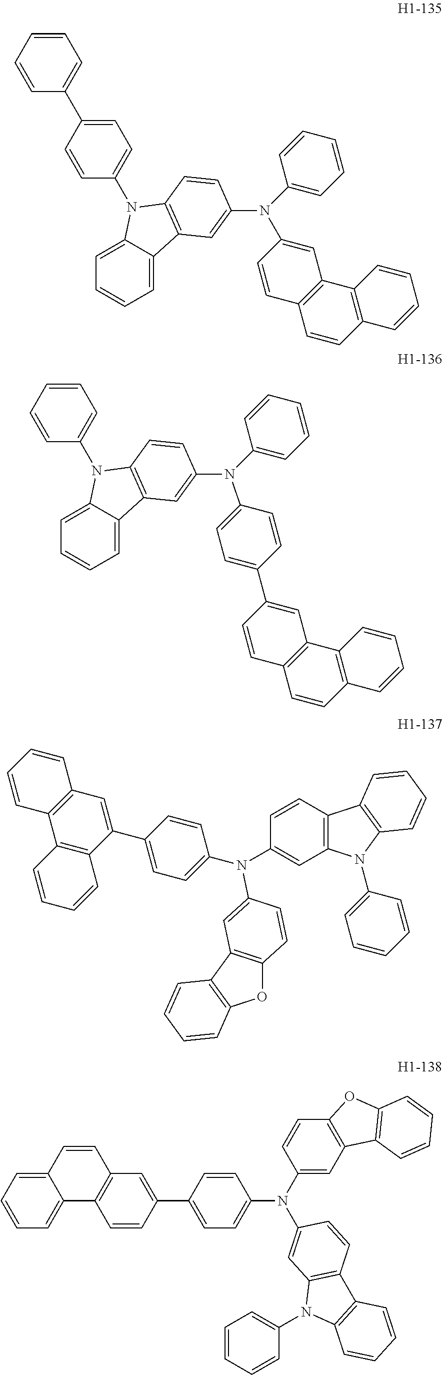 [SMILES: c1ccc(-c2ccc(-n3c4ccccc4c4cc(N(c5ccccc5)c5ccc6ccc7ccccc7c6c5)ccc43)cc2)cc1 ; c1ccc(N(c2ccc(-c3ccc4ccc5ccccc5c4c3)cc2)c2ccc3c(c2)c2ccccc2n3-c2ccccc2)cc1 ; c1ccc(-n2c3ccccc3c3ccc(N(c4ccc(-c5cc6ccccc6c6ccccc56)cc4)c4ccc5oc6ccccc6c5c4)cc32)cc1 ; c1ccc(-n2c3ccccc3c3ccc(N(c4ccc(-c5ccc6c(ccc7ccccc76)c5)cc4)c4ccc5oc6ccccc6c5c4)cc32)cc1]

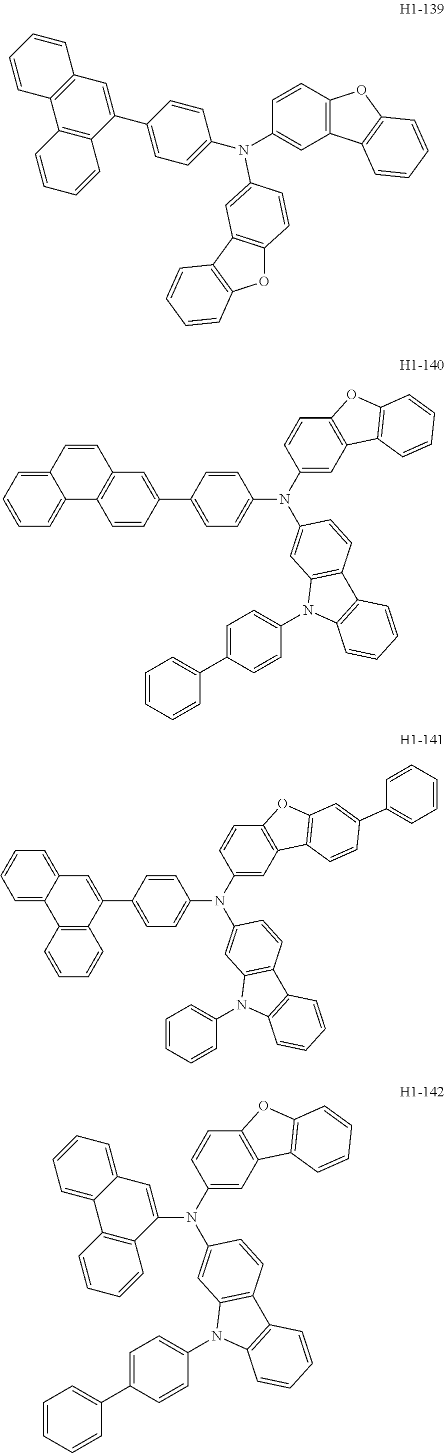 [SMILES: c1ccc2c(c1)cc(-c1ccc(N(c3ccc4oc5ccccc5c4c3)c3ccc4oc5ccccc5c4c3)cc1)c1ccccc12 ; c1ccc(-c2ccc(-n3c4ccccc4c4ccc(N(c5ccc(-c6ccc7c(ccc8ccccc87)c6)cc5)c5ccc6oc7ccccc7c6c5)cc43)cc2)cc1 ; c1ccc(-c2ccc3c(c2)oc2ccc(N(c4ccc(-c5cc6ccccc6c6ccccc56)cc4)c4ccc5c6ccccc6n(-c6ccccc6)c5c4)cc23)cc1 ; c1ccc(-c2ccc(-n3c4ccccc4c4ccc(N(c5ccc6oc7ccccc7c6c5)c5cc6ccccc6c6ccccc56)cc43)cc2)cc1]

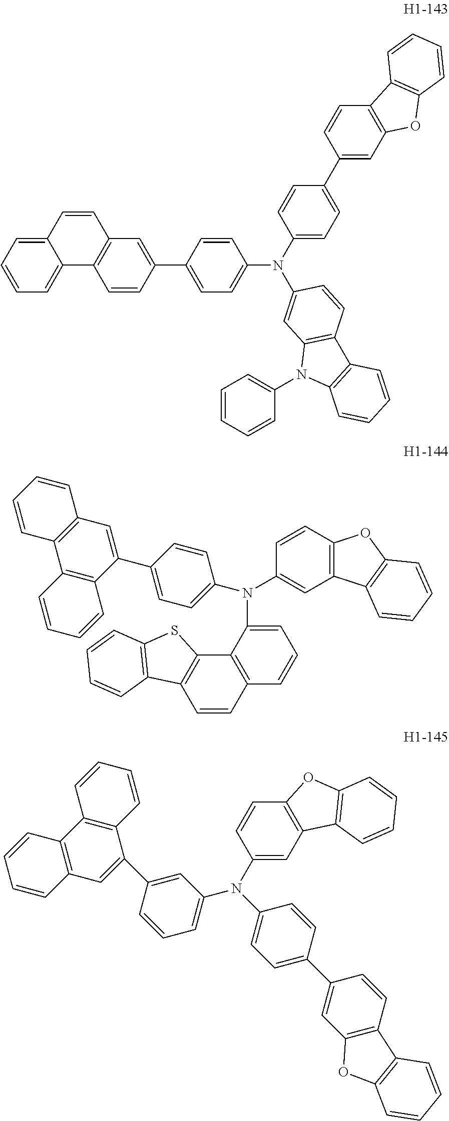 [SMILES: c1ccc(-n2c3ccccc3c3ccc(N(c4ccc(-c5ccc6c(ccc7ccccc76)c5)cc4)c4ccc(-c5ccc6c(c5)oc5ccccc56)cc4)cc32)cc1 ; c1ccc2c(c1)cc(-c1ccc(N(c3ccc4oc5ccccc5c4c3)c3cccc4ccc5c6ccccc6sc5c34)cc1)c1ccccc12 ; c1cc(-c2cc3ccccc3c3ccccc23)cc(N(c2ccc(-c3ccc4c(c3)oc3ccccc34)cc2)c2ccc3oc4ccccc4c3c2)c1]

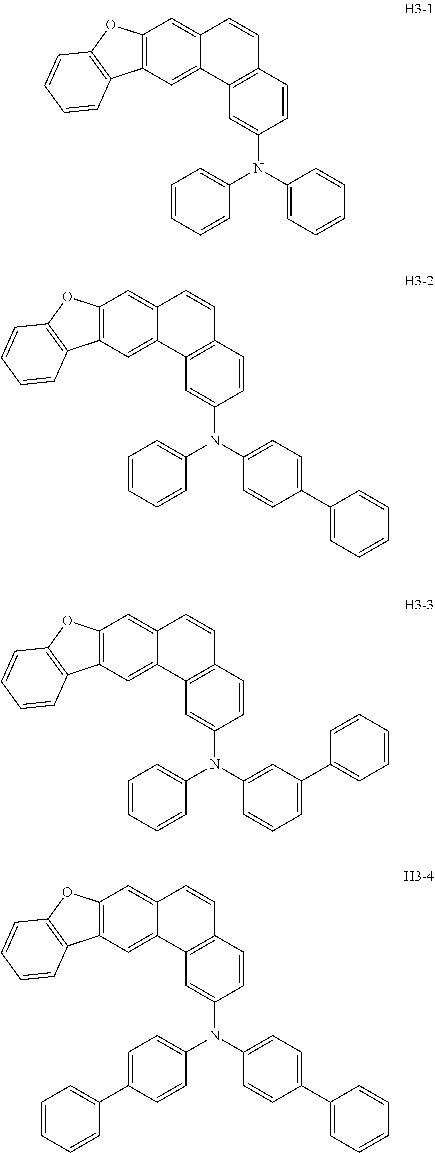 [SMILES: c1ccc(N(c2ccccc2)c2ccc3ccc4cc5oc6ccccc6c5cc4c3c2)cc1 ; c1ccc(-c2ccc(N(c3ccccc3)c3ccc4ccc5cc6oc7ccccc7c6cc5c4c3)cc2)cc1 ; c1ccc(-c2cccc(N(c3ccccc3)c3ccc4ccc5cc6oc7ccccc7c6cc5c4c3)c2)cc1 ; c1ccc(-c2ccc(N(c3ccc(-c4ccccc4)cc3)c3ccc4ccc5cc6oc7ccccc7c6cc5c4c3)cc2)cc1]

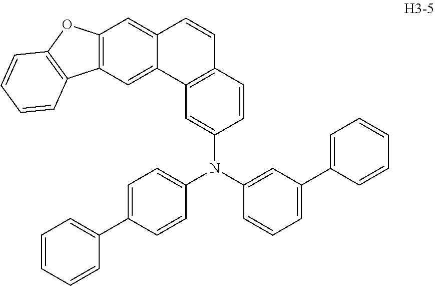 [SMILES: c1ccc(-c2ccc(N(c3cccc(-c4ccccc4)c3)c3ccc4ccc5cc6oc7ccccc7c6cc5c4c3)cc2)cc1]

7. The organic electroluminescent device according to claim 3, wherein the compound represented by Formula 2 is selected from the following compounds: 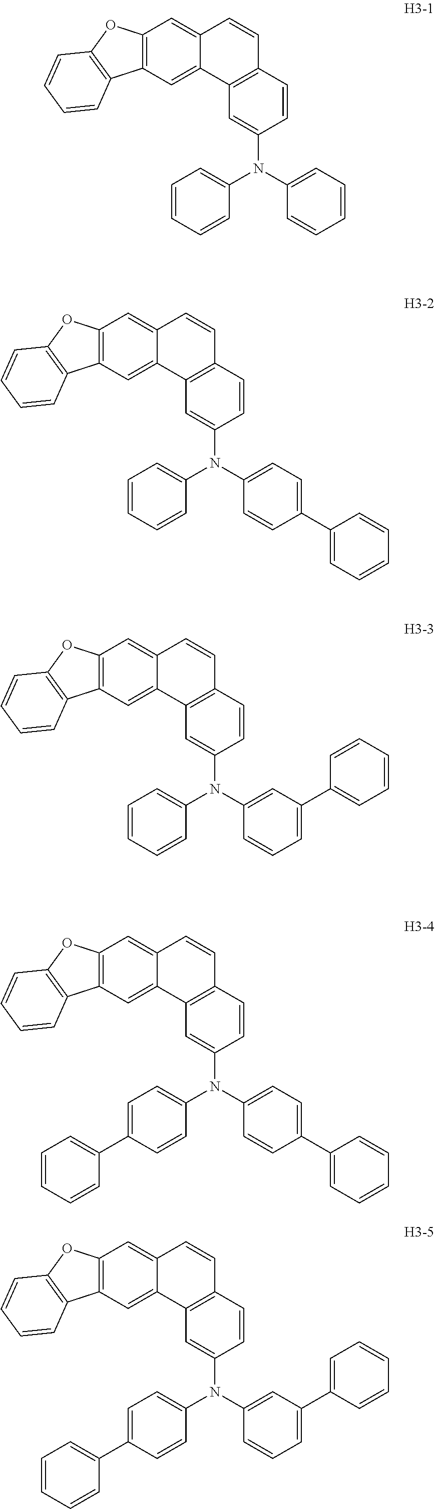 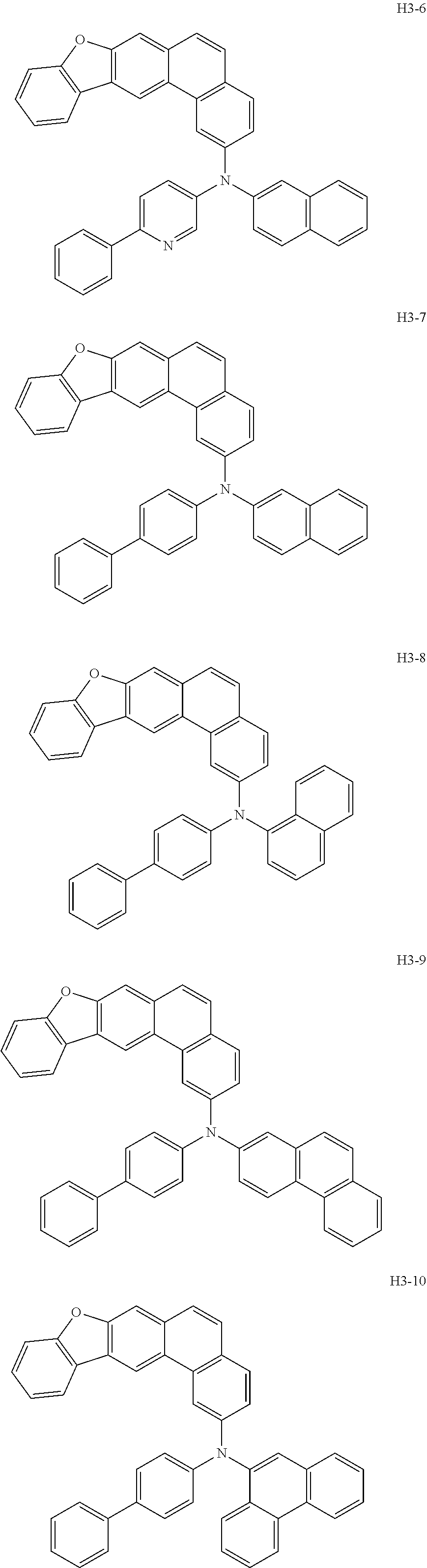 [SMILES: c1ccc(-c2ccc(N(c3ccc4ccccc4c3)c3ccc4ccc5cc6oc7ccccc7c6cc5c4c3)cn2)cc1 ; c1ccc(-c2ccc(N(c3ccc4ccccc4c3)c3ccc4ccc5cc6oc7ccccc7c6cc5c4c3)cc2)cc1 ; c1ccc(-c2ccc(N(c3ccc4ccc5cc6oc7ccccc7c6cc5c4c3)c3cccc4ccccc34)cc2)cc1 ; c1ccc(-c2ccc(N(c3ccc4c(ccc5ccccc54)c3)c3ccc4ccc5cc6oc7ccccc7c6cc5c4c3)cc2)cc1 ; c1ccc(-c2ccc(N(c3ccc4ccc5cc6oc7ccccc7c6cc5c4c3)c3cc4ccccc4c4ccccc34)cc2)cc1] 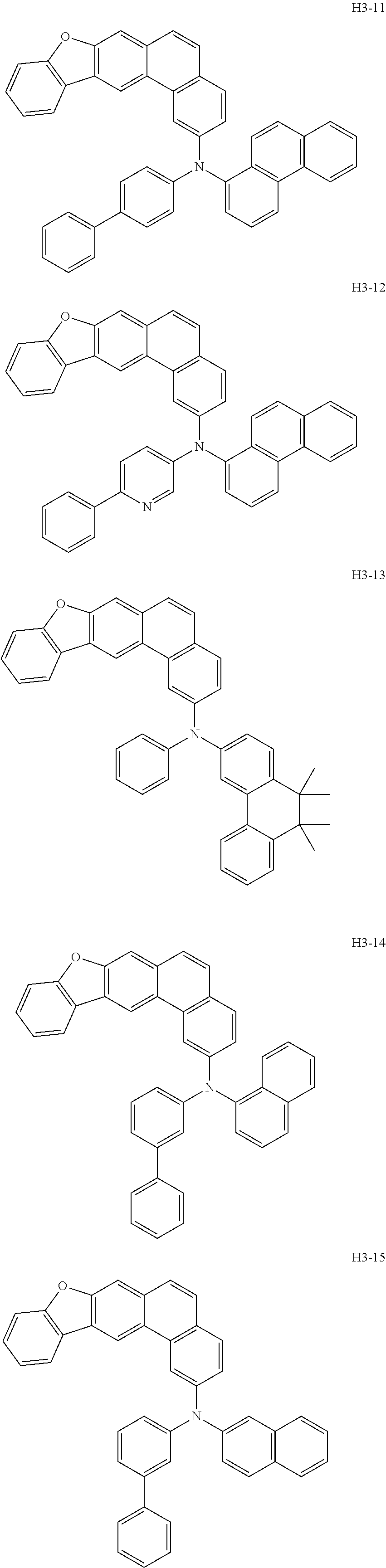 [SMILES: c1ccc(-c2ccc(N(c3ccc4ccc5cc6oc7ccccc7c6cc5c4c3)c3cccc4c3ccc3ccccc34)cc2)cc1 ; c1ccc(-c2ccc(N(c3ccc4ccc5cc6oc7ccccc7c6cc5c4c3)c3cccc4c3ccc3ccccc34)cn2)cc1 ; CC1(C)c2ccccc2-c2cc(N(c3ccccc3)c3ccc4ccc5cc6oc7ccccc7c6cc5c4c3)ccc2C1(C)C ; c1ccc(-c2cccc(N(c3ccc4ccc5cc6oc7ccccc7c6cc5c4c3)c3cccc4ccccc34)c2)cc1 ; c1ccc(-c2cccc(N(c3ccc4ccccc4c3)c3ccc4ccc5cc6oc7ccccc7c6cc5c4c3)c2)cc1] 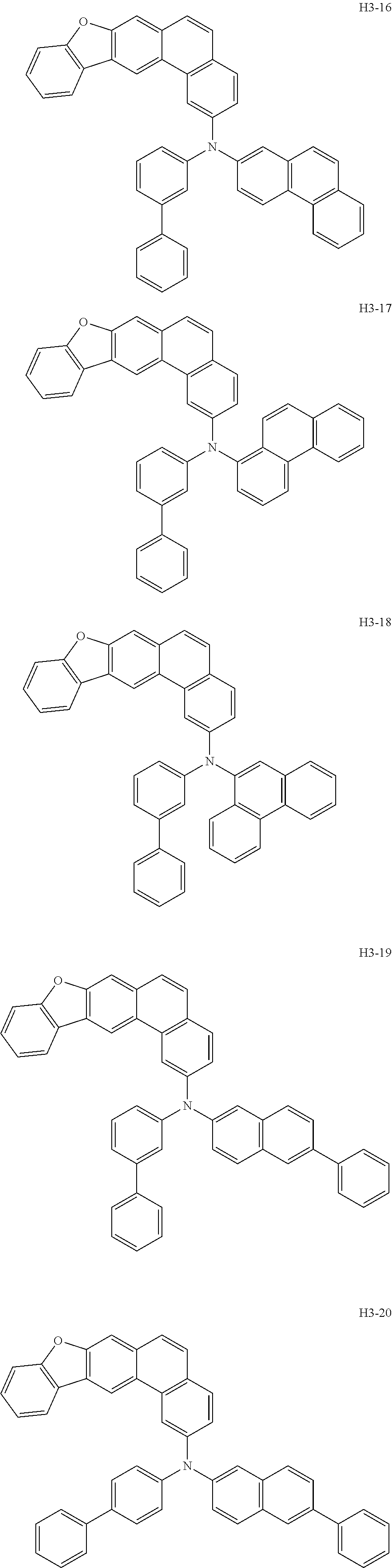 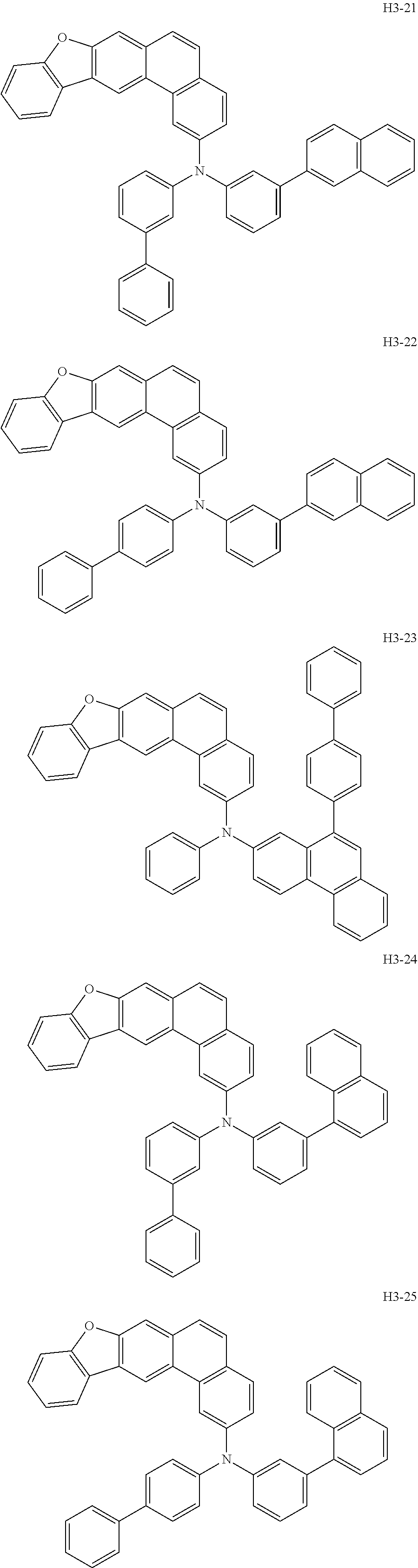 [SMILES: c1ccc(-c2cccc(N(c3cccc(-c4ccc5ccccc5c4)c3)c3ccc4ccc5cc6oc7ccccc7c6cc5c4c3)c2)cc1 ; c1ccc(-c2ccc(N(c3cccc(-c4ccc5ccccc5c4)c3)c3ccc4ccc5cc6oc7ccccc7c6cc5c4c3)cc2)cc1 ; c1ccc(-c2ccc(-c3cc4ccccc4c4ccc(N(c5ccccc5)c5ccc6ccc7cc8oc9ccccc9c8cc7c6c5)cc34)cc2)cc1 ; c1ccc(-c2cccc(N(c3cccc(-c4cccc5ccccc45)c3)c3ccc4ccc5cc6oc7ccccc7c6cc5c4c3)c2)cc1 ; c1ccc(-c2ccc(N(c3cccc(-c4cccc5ccccc45)c3)c3ccc4ccc5cc6oc7ccccc7c6cc5c4c3)cc2)cc1] 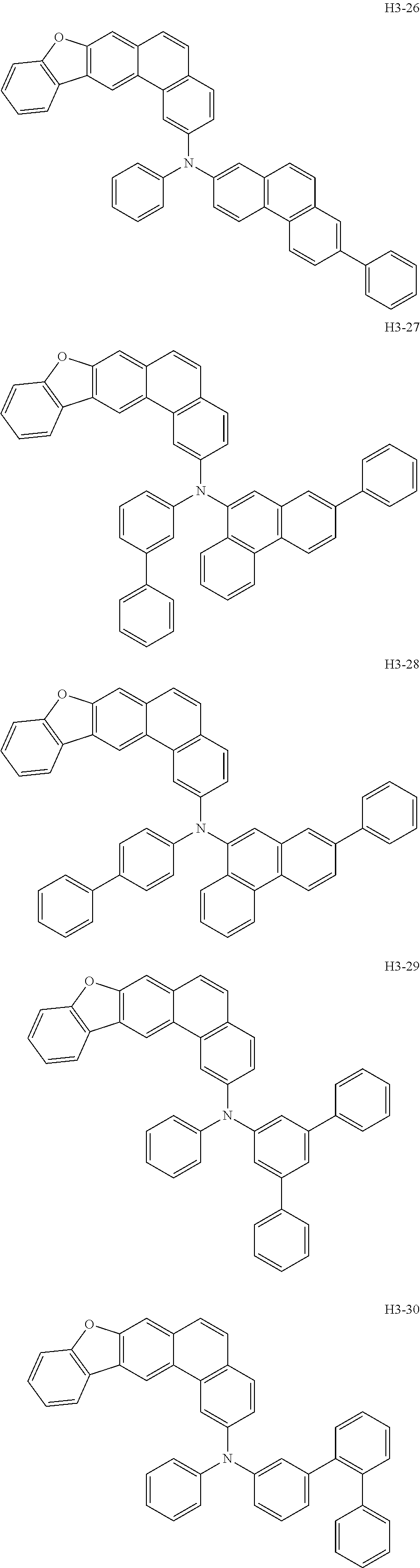 [SMILES: c1ccc(-c2ccc3c(ccc4cc(N(c5ccccc5)c5ccc6ccc7cc8oc9ccccc9c8cc7c6c5)ccc43)c2)cc1 ; c1ccc(-c2cccc(N(c3ccc4ccc5cc6oc7ccccc7c6cc5c4c3)c3cc4cc(-c5ccccc5)ccc4c4ccccc34)c2)cc1 ; c1ccc(-c2ccc(N(c3ccc4ccc5cc6oc7ccccc7c6cc5c4c3)c3cc4cc(-c5ccccc5)ccc4c4ccccc34)cc2)cc1 ; c1ccc(-c2cc(-c3ccccc3)cc(N(c3ccccc3)c3ccc4ccc5cc6oc7ccccc7c6cc5c4c3)c2)cc1 ; c1ccc(-c2ccccc2-c2cccc(N(c3ccccc3)c3ccc4ccc5cc6oc7ccccc7c6cc5c4c3)c2)cc1] 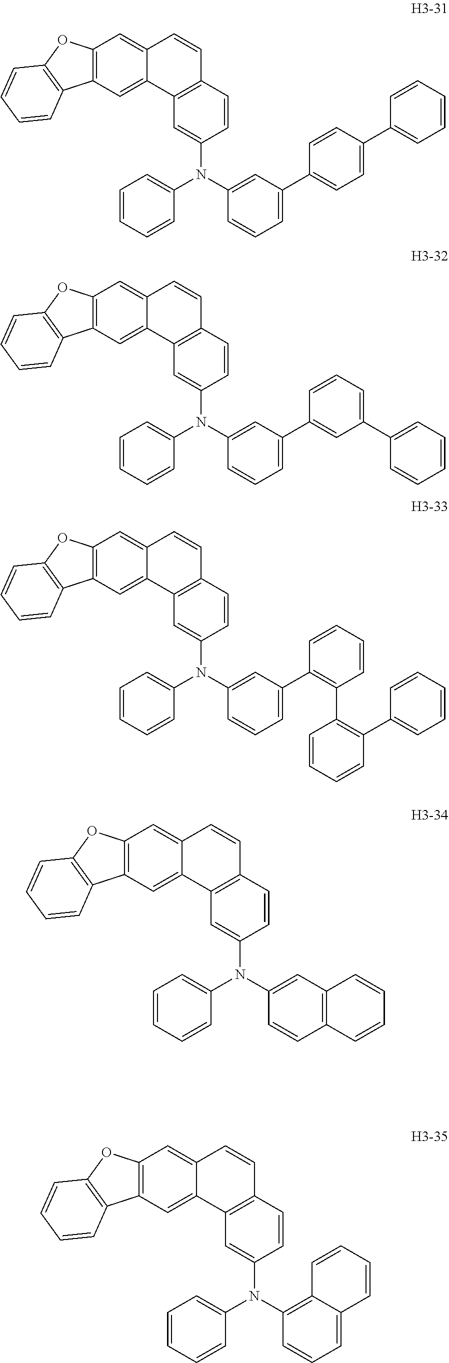 [SMILES: c1ccc(-c2ccc(-c3cccc(N(c4ccccc4)c4ccc5ccc6cc7oc8ccccc8c7cc6c5c4)c3)cc2)cc1 ; c1ccc(-c2cccc(-c3cccc(N(c4ccccc4)c4ccc5ccc6cc7oc8ccccc8c7cc6c5c4)c3)c2)cc1 ; c1ccc(-c2ccccc2-c2ccccc2-c2cccc(N(c3ccccc3)c3ccc4ccc5cc6oc7ccccc7c6cc5c4c3)c2)cc1 ; c1ccc(N(c2ccc3ccccc3c2)c2ccc3ccc4cc5oc6ccccc6c5cc4c3c2)cc1 ; c1ccc(N(c2ccc3ccc4cc5oc6ccccc6c5cc4c3c2)c2cccc3ccccc23)cc1] 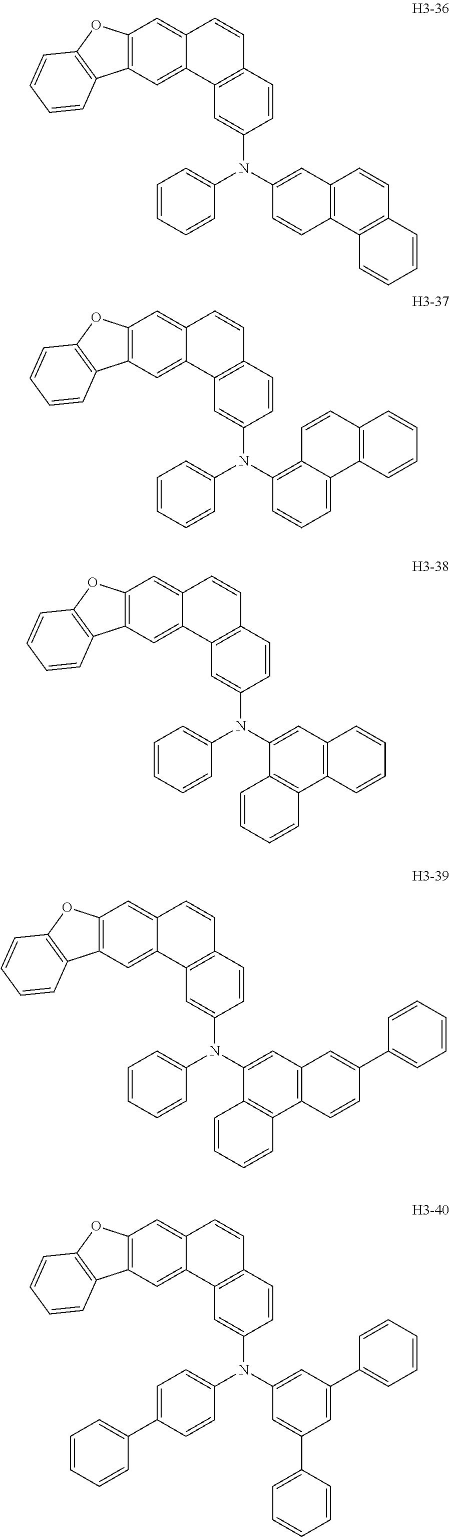 [SMILES: c1ccc(N(c2ccc3c(ccc4ccccc43)c2)c2ccc3ccc4cc5oc6ccccc6c5cc4c3c2)cc1 ; c1ccc(N(c2ccc3ccc4cc5oc6ccccc6c5cc4c3c2)c2cccc3c2ccc2ccccc23)cc1 ; c1ccc(N(c2ccc3ccc4cc5oc6ccccc6c5cc4c3c2)c2cc3ccccc3c3ccccc23)cc1 ; c1ccc(-c2ccc3c(c2)cc(N(c2ccccc2)c2ccc4ccc5cc6oc7ccccc7c6cc5c4c2)c2ccccc23)cc1 ; c1ccc(-c2ccc(N(c3cc(-c4ccccc4)cc(-c4ccccc4)c3)c3ccc4ccc5cc6oc7ccccc7c6cc5c4c3)cc2)cc1] 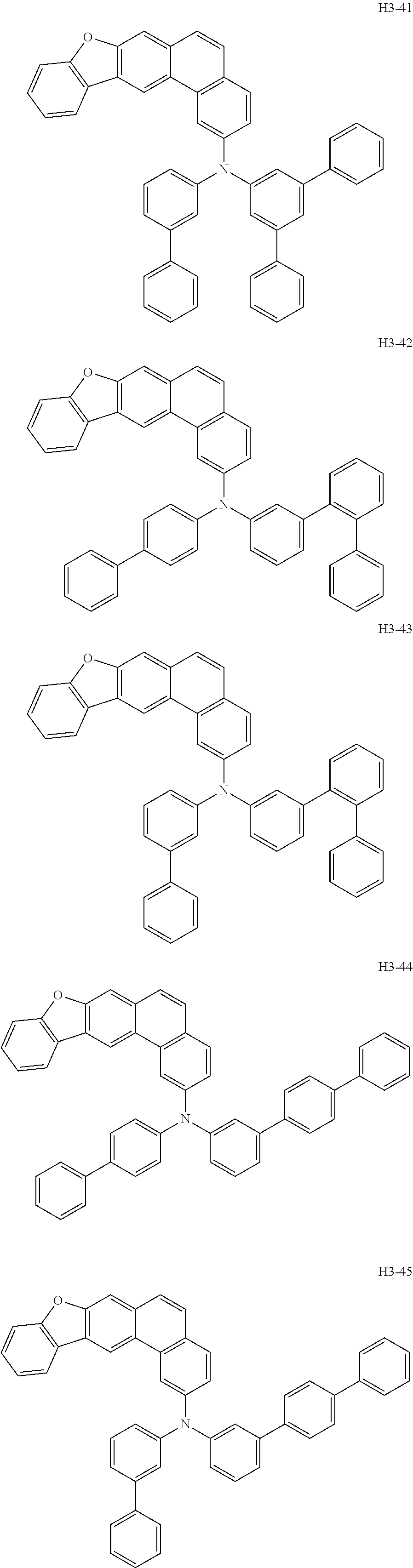 [SMILES: c1ccc(-c2cccc(N(c3cc(-c4ccccc4)cc(-c4ccccc4)c3)c3ccc4ccc5cc6oc7ccccc7c6cc5c4c3)c2)cc1 ; c1ccc(-c2ccc(N(c3cccc(-c4ccccc4-c4ccccc4)c3)c3ccc4ccc5cc6oc7ccccc7c6cc5c4c3)cc2)cc1 ; c1ccc(-c2cccc(N(c3cccc(-c4ccccc4-c4ccccc4)c3)c3ccc4ccc5cc6oc7ccccc7c6cc5c4c3)c2)cc1 ; c1ccc(-c2ccc(-c3cccc(N(c4ccc(-c5ccccc5)cc4)c4ccc5ccc6cc7oc8ccccc8c7cc6c5c4)c3)cc2)cc1 ; c1ccc(-c2ccc(-c3cccc(N(c4cccc(-c5ccccc5)c4)c4ccc5ccc6cc7oc8ccccc8c7cc6c5c4)c3)cc2)cc1] 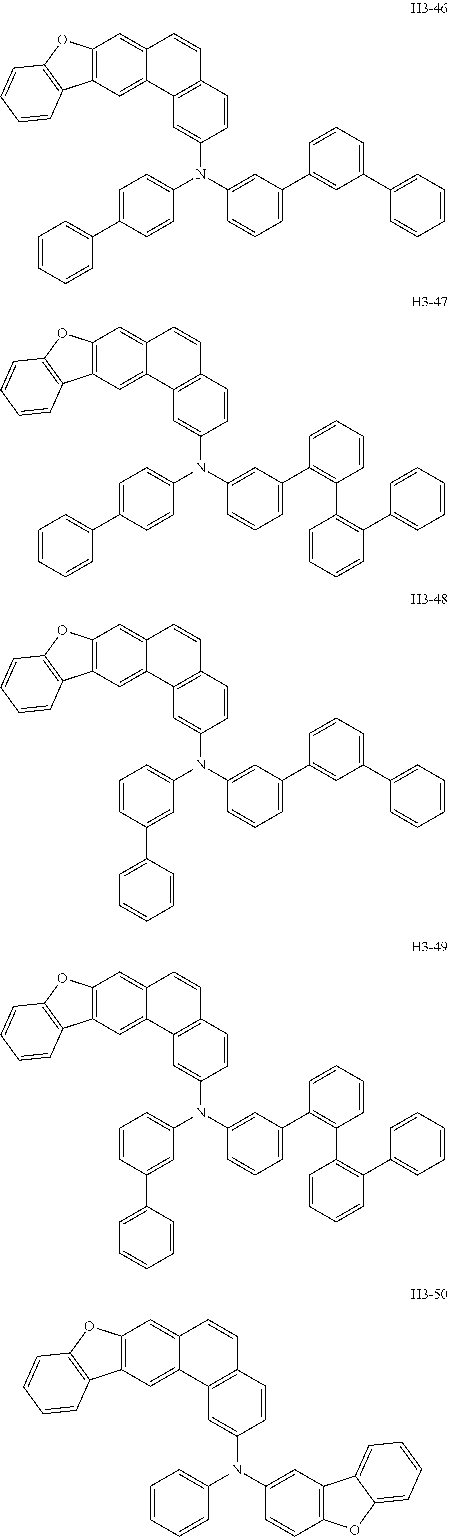 [SMILES: c1ccc(-c2ccc(N(c3cccc(-c4cccc(-c5ccccc5)c4)c3)c3ccc4ccc5cc6oc7ccccc7c6cc5c4c3)cc2)cc1 ; c1ccc(-c2ccc(N(c3cccc(-c4ccccc4-c4ccccc4-c4ccccc4)c3)c3ccc4ccc5cc6oc7ccccc7c6cc5c4c3)cc2)cc1 ; c1ccc(-c2cccc(-c3cccc(N(c4cccc(-c5ccccc5)c4)c4ccc5ccc6cc7oc8ccccc8c7cc6c5c4)c3)c2)cc1 ; c1ccc(-c2cccc(N(c3cccc(-c4ccccc4-c4ccccc4-c4ccccc4)c3)c3ccc4ccc5cc6oc7ccccc7c6cc5c4c3)c2)cc1 ; c1ccc(N(c2ccc3ccc4cc5oc6ccccc6c5cc4c3c2)c2ccc3oc4ccccc4c3c2)cc1] 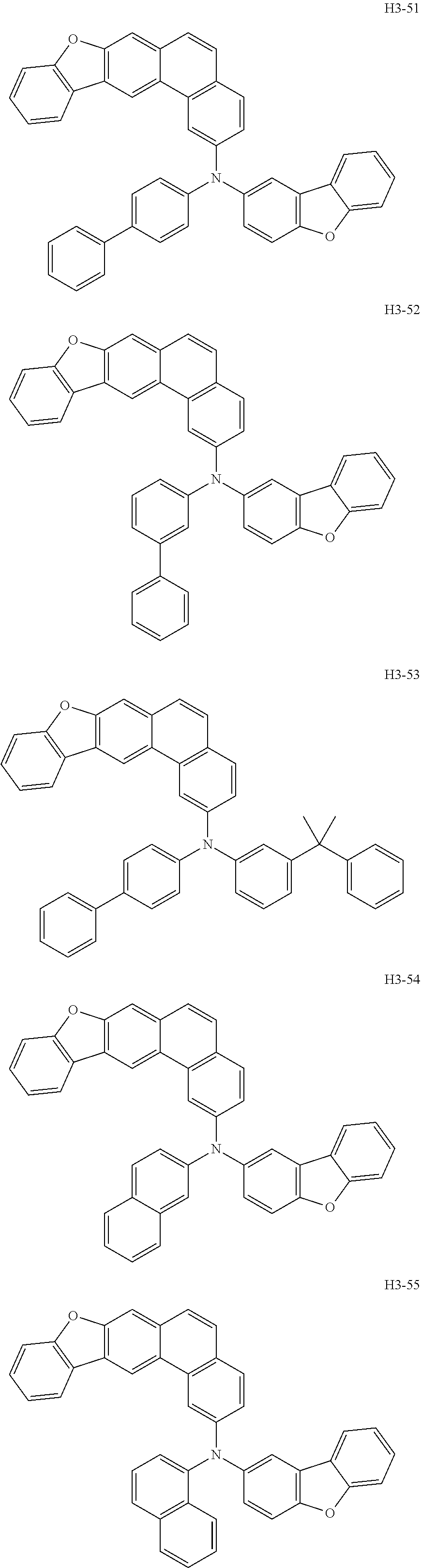 [SMILES: c1ccc(-c2ccc(N(c3ccc4ccc5cc6oc7ccccc7c6cc5c4c3)c3ccc4oc5ccccc5c4c3)cc2)cc1 ; c1ccc(-c2cccc(N(c3ccc4ccc5cc6oc7ccccc7c6cc5c4c3)c3ccc4oc5ccccc5c4c3)c2)cc1 ; CC(C)(c1ccccc1)c1cccc(N(c2ccc(-c3ccccc3)cc2)c2ccc3ccc4cc5oc6ccccc6c5cc4c3c2)c1 ; c1ccc2cc(N(c3ccc4ccc5cc6oc7ccccc7c6cc5c4c3)c3ccc4oc5ccccc5c4c3)ccc2c1 ; c1ccc2c(N(c3ccc4ccc5cc6oc7ccccc7c6cc5c4c3)c3ccc4oc5ccccc5c4c3)cccc2c1] 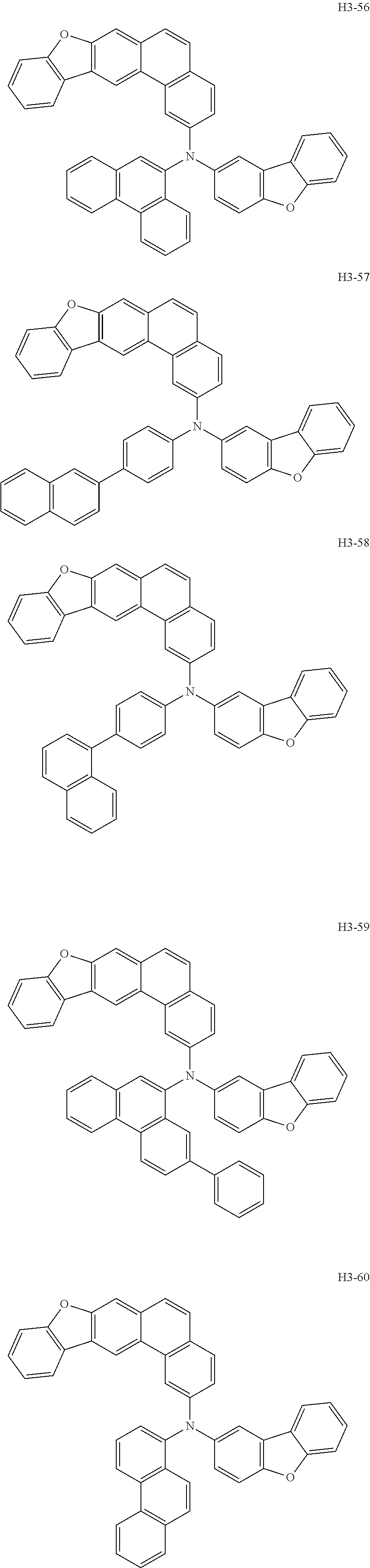 [SMILES: c1ccc2c(c1)cc(N(c1ccc3ccc4cc5oc6ccccc6c5cc4c3c1)c1ccc3oc4ccccc4c3c1)c1ccccc12 ; c1ccc2cc(-c3ccc(N(c4ccc5ccc6cc7oc8ccccc8c7cc6c5c4)c4ccc5oc6ccccc6c5c4)cc3)ccc2c1 ; c1ccc2c(-c3ccc(N(c4ccc5ccc6cc7oc8ccccc8c7cc6c5c4)c4ccc5oc6ccccc6c5c4)cc3)cccc2c1 ; c1ccc(-c2ccc3c(c2)c(N(c2ccc4ccc5cc6oc7ccccc7c6cc5c4c2)c2ccc4oc5ccccc5c4c2)cc2ccccc23)cc1 ; c1ccc2c(c1)ccc1c(N(c3ccc4ccc5cc6oc7ccccc7c6cc5c4c3)c3ccc4oc5ccccc5c4c3)cccc12] 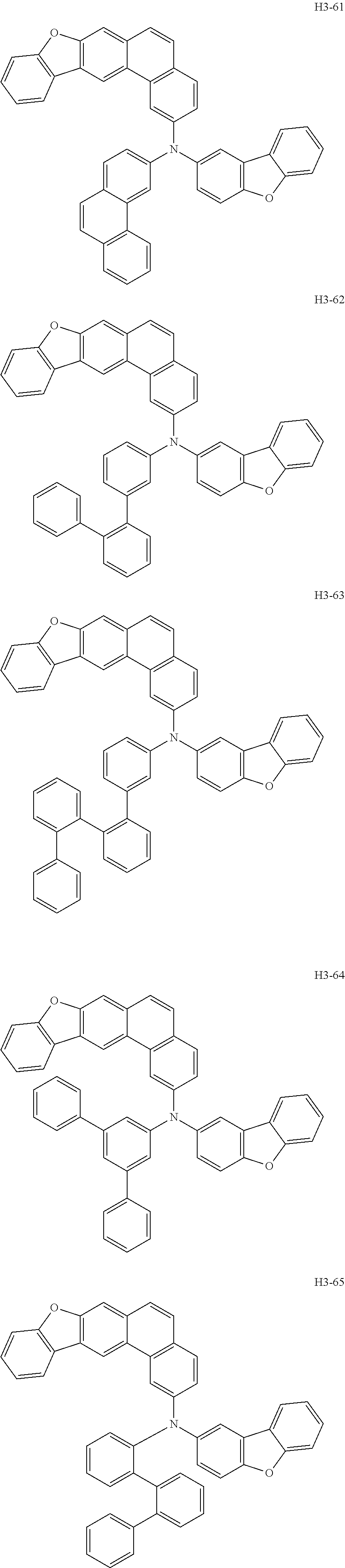 [SMILES: c1ccc2c(c1)ccc1ccc(N(c3ccc4ccc5cc6oc7ccccc7c6cc5c4c3)c3ccc4oc5ccccc5c4c3)cc12 ; c1ccc(-c2ccccc2-c2cccc(N(c3ccc4ccc5cc6oc7ccccc7c6cc5c4c3)c3ccc4oc5ccccc5c4c3)c2)cc1 ; c1ccc(-c2ccccc2-c2ccccc2-c2cccc(N(c3ccc4ccc5cc6oc7ccccc7c6cc5c4c3)c3ccc4oc5ccccc5c4c3)c2)cc1 ; c1ccc(-c2cc(-c3ccccc3)cc(N(c3ccc4ccc5cc6oc7ccccc7c6cc5c4c3)c3ccc4oc5ccccc5c4c3)c2)cc1 ; c1ccc(-c2ccccc2-c2ccccc2N(c2ccc3ccc4cc5oc6ccccc6c5cc4c3c2)c2ccc3oc4ccccc4c3c2)cc1] 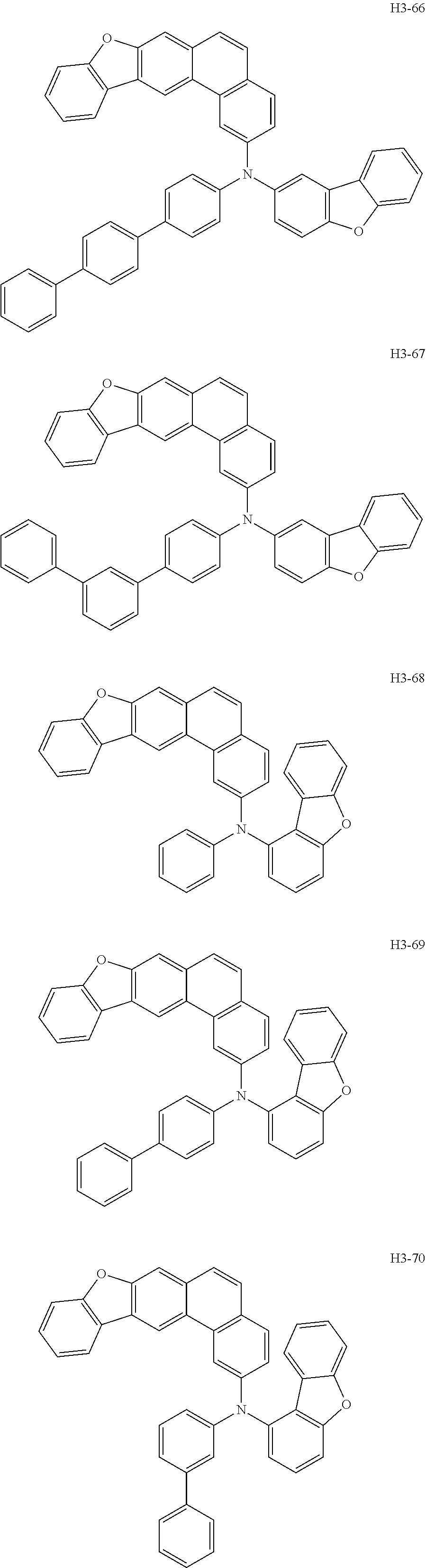 [SMILES: c1ccc(-c2ccc(-c3ccc(N(c4ccc5ccc6cc7oc8ccccc8c7cc6c5c4)c4ccc5oc6ccccc6c5c4)cc3)cc2)cc1 ; c1ccc(-c2cccc(-c3ccc(N(c4ccc5ccc6cc7oc8ccccc8c7cc6c5c4)c4ccc5oc6ccccc6c5c4)cc3)c2)cc1 ; c1ccc(N(c2ccc3ccc4cc5oc6ccccc6c5cc4c3c2)c2cccc3oc4ccccc4c23)cc1 ; c1ccc(-c2ccc(N(c3ccc4ccc5cc6oc7ccccc7c6cc5c4c3)c3cccc4oc5ccccc5c34)cc2)cc1 ; c1ccc(-c2cccc(N(c3ccc4ccc5cc6oc7ccccc7c6cc5c4c3)c3cccc4oc5ccccc5c34)c2)cc1] 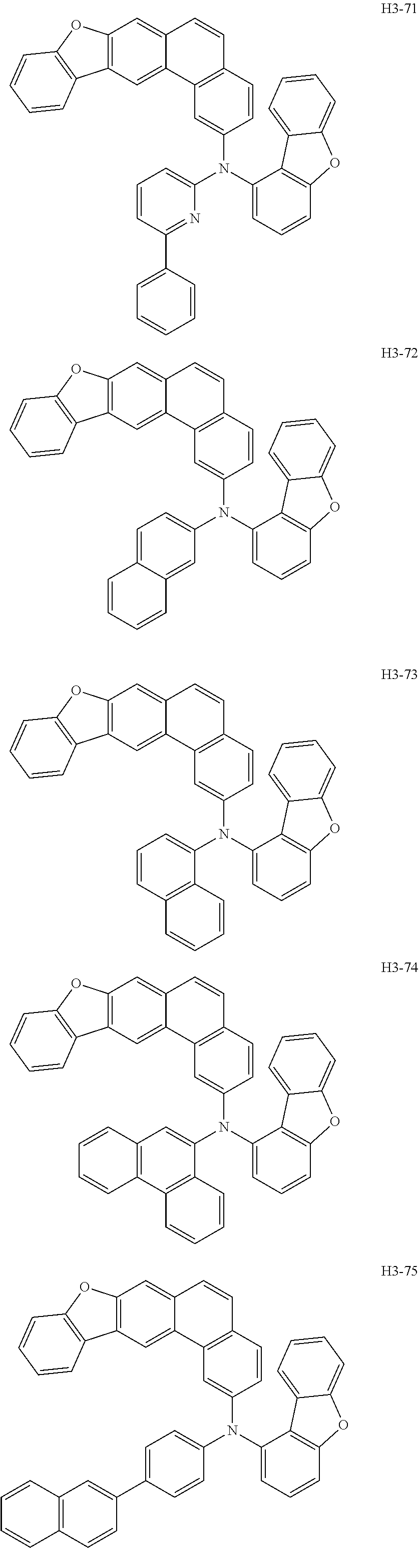 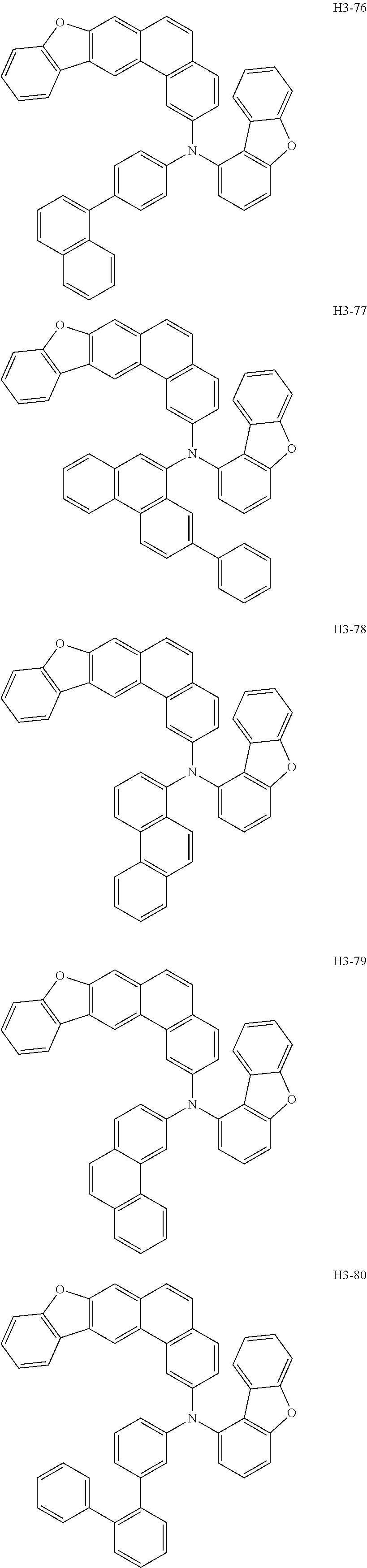 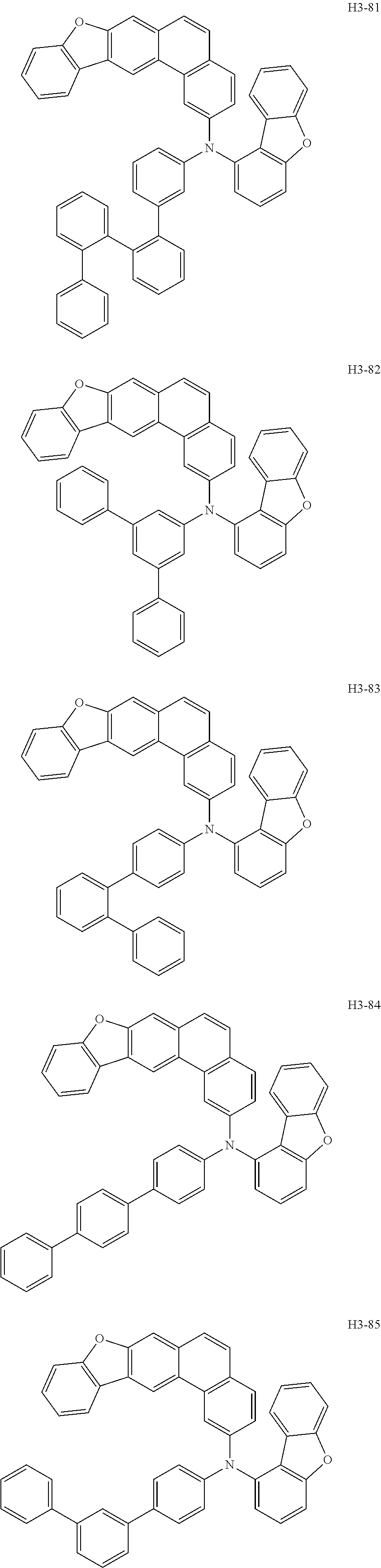 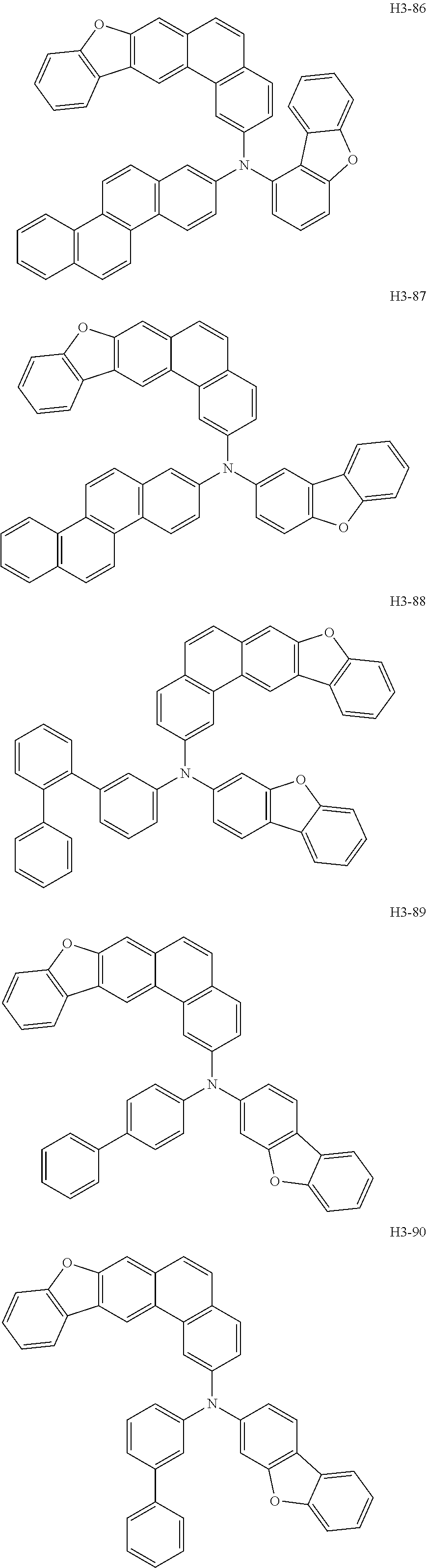 [SMILES: c1ccc2c(c1)ccc1c3ccc(N(c4ccc5ccc6cc7oc8ccccc8c7cc6c5c4)c4cccc5oc6ccccc6c45)cc3ccc21 ; c1ccc2c(c1)ccc1c3ccc(N(c4ccc5ccc6cc7oc8ccccc8c7cc6c5c4)c4ccc5oc6ccccc6c5c4)cc3ccc21 ; c1ccc(-c2ccccc2-c2cccc(N(c3ccc4c(c3)oc3ccccc34)c3ccc4ccc5cc6oc7ccccc7c6cc5c4c3)c2)cc1 ; c1ccc(-c2ccc(N(c3ccc4c(c3)oc3ccccc34)c3ccc4ccc5cc6oc7ccccc7c6cc5c4c3)cc2)cc1 ; c1ccc(-c2cccc(N(c3ccc4c(c3)oc3ccccc34)c3ccc4ccc5cc6oc7ccccc7c6cc5c4c3)c2)cc1] 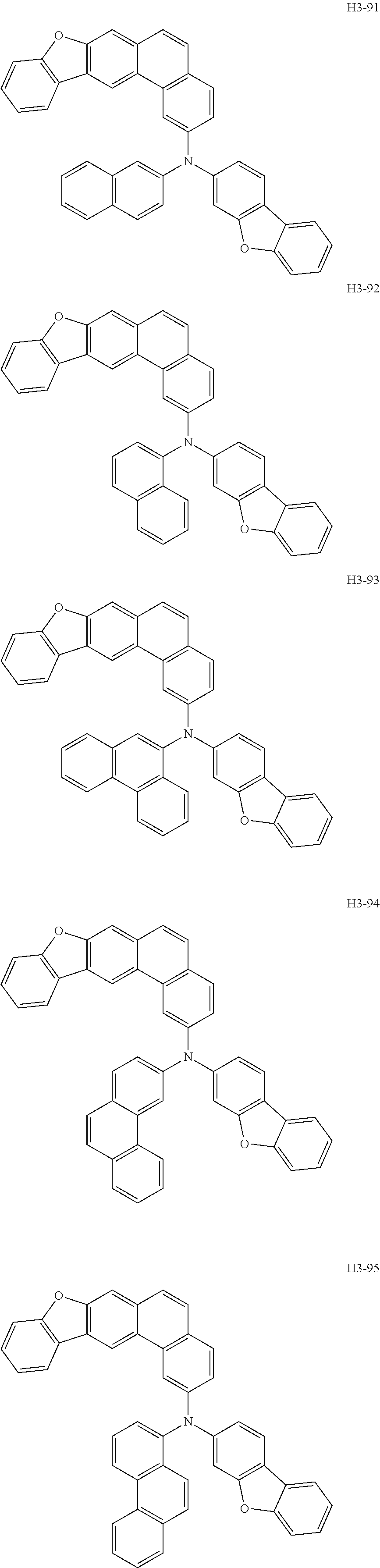 [SMILES: c1ccc2cc(N(c3ccc4c(c3)oc3ccccc34)c3ccc4ccc5cc6oc7ccccc7c6cc5c4c3)ccc2c1 ; c1ccc2c(N(c3ccc4c(c3)oc3ccccc34)c3ccc4ccc5cc6oc7ccccc7c6cc5c4c3)cccc2c1 ; c1ccc2c(c1)cc(N(c1ccc3c(c1)oc1ccccc13)c1ccc3ccc4cc5oc6ccccc6c5cc4c3c1)c1ccccc12 ; c1ccc2c(c1)ccc1ccc(N(c3ccc4c(c3)oc3ccccc34)c3ccc4ccc5cc6oc7ccccc7c6cc5c4c3)cc12 ; c1ccc2c(c1)ccc1c(N(c3ccc4c(c3)oc3ccccc34)c3ccc4ccc5cc6oc7ccccc7c6cc5c4c3)cccc12] 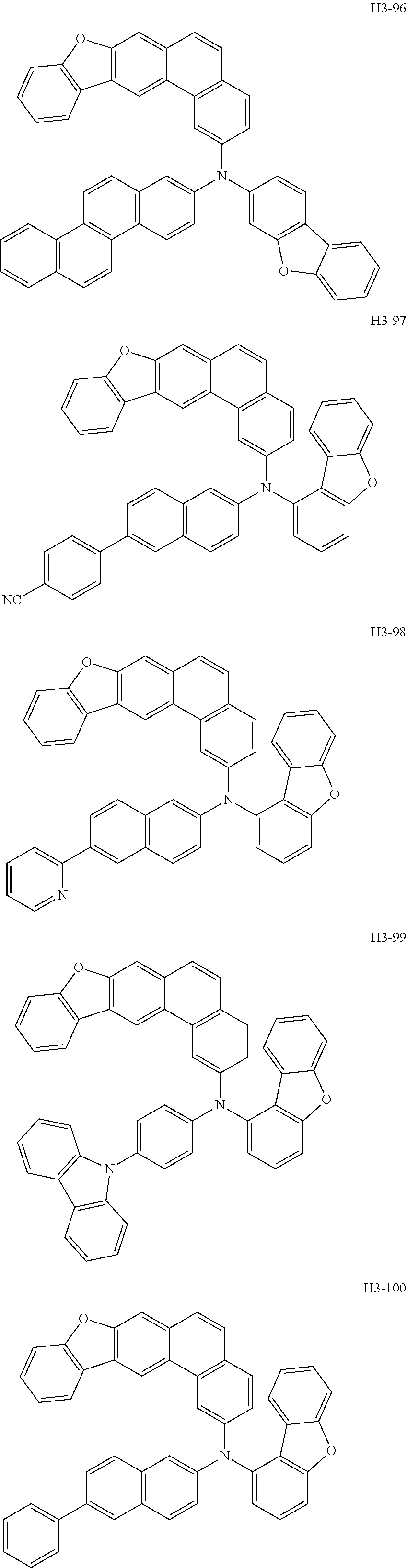 [SMILES: c1ccc2c(c1)ccc1c3ccc(N(c4ccc5c(c4)oc4ccccc45)c4ccc5ccc6cc7oc8ccccc8c7cc6c5c4)cc3ccc21 ; N#Cc1ccc(-c2ccc3cc(N(c4ccc5ccc6cc7oc8ccccc8c7cc6c5c4)c4cccc5oc6ccccc6c45)ccc3c2)cc1 ; c1ccc(-c2ccc3cc(N(c4ccc5ccc6cc7oc8ccccc8c7cc6c5c4)c4cccc5oc6ccccc6c45)ccc3c2)nc1 ; c1ccc2c(c1)oc1cc3ccc4ccc(N(c5ccc(-n6c7ccccc7c7ccccc76)cc5)c5cccc6oc7ccccc7c56)cc4c3cc12 ; c1ccc(-c2ccc3cc(N(c4ccc5ccc6cc7oc8ccccc8c7cc6c5c4)c4cccc5oc6ccccc6c45)ccc3c2)cc1] 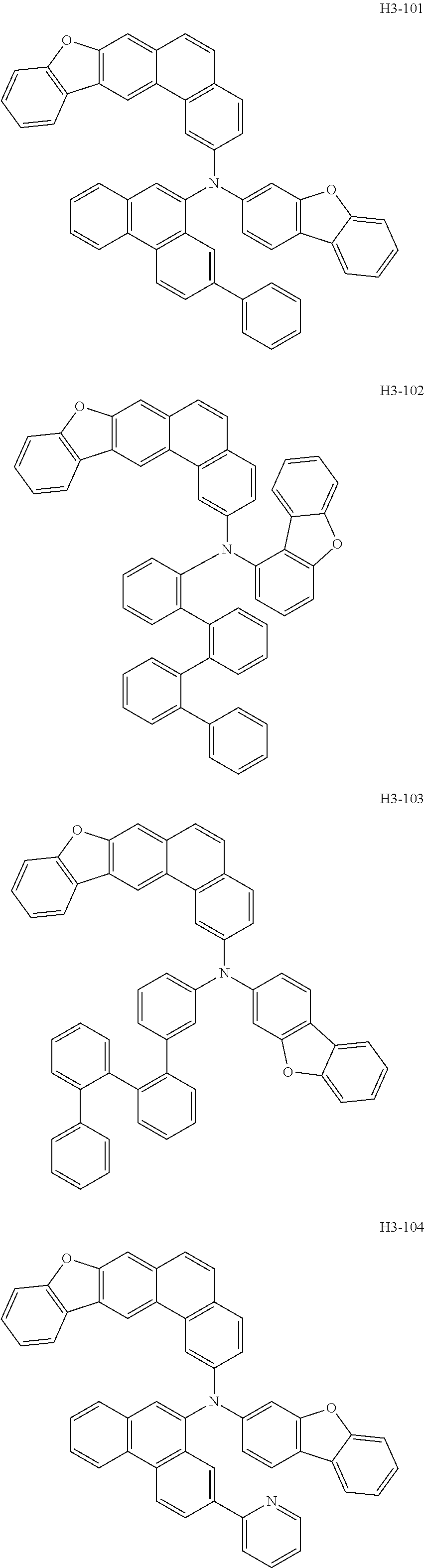 [SMILES: c1ccc(-c2ccc3c(c2)c(N(c2ccc4c(c2)oc2ccccc24)c2ccc4ccc5cc6oc7ccccc7c6cc5c4c2)cc2ccccc23)cc1 ; c1ccc(-c2ccccc2-c2ccccc2-c2ccccc2N(c2ccc3ccc4cc5oc6ccccc6c5cc4c3c2)c2cccc3oc4ccccc4c23)cc1 ; c1ccc(-c2ccccc2-c2ccccc2-c2cccc(N(c3ccc4c(c3)oc3ccccc34)c3ccc4ccc5cc6oc7ccccc7c6cc5c4c3)c2)cc1 ; c1ccc(-c2ccc3c(c2)c(N(c2ccc4c(c2)oc2ccccc24)c2ccc4ccc5cc6oc7ccccc7c6cc5c4c2)cc2ccccc23)nc1] 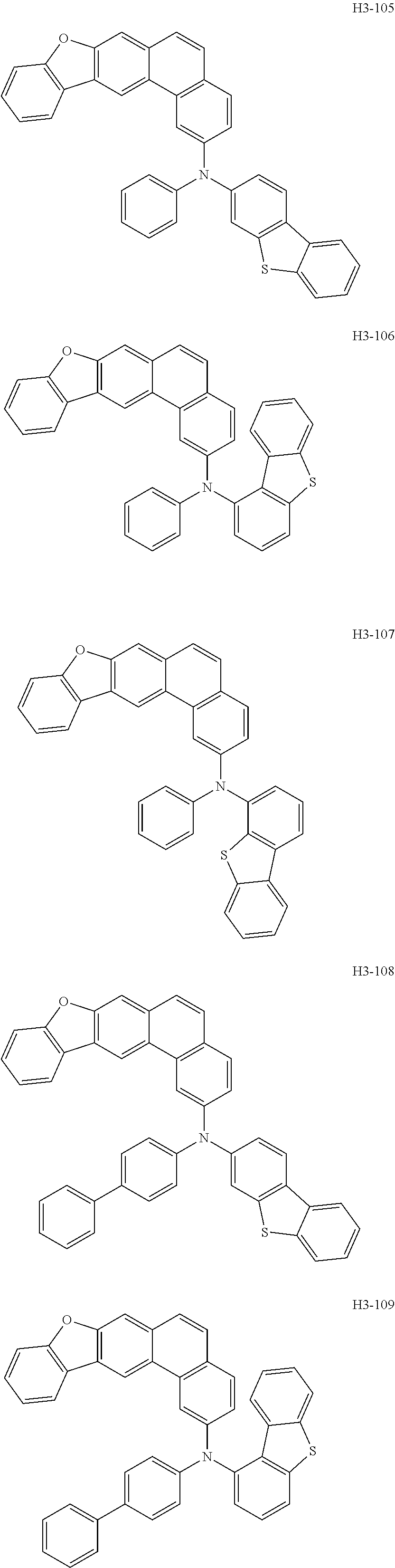 [SMILES: c1ccc(N(c2ccc3c(c2)sc2ccccc23)c2ccc3ccc4cc5oc6ccccc6c5cc4c3c2)cc1 ; c1ccc(N(c2ccc3ccc4cc5oc6ccccc6c5cc4c3c2)c2cccc3sc4ccccc4c23)cc1 ; c1ccc(N(c2ccc3ccc4cc5oc6ccccc6c5cc4c3c2)c2cccc3c2sc2ccccc23)cc1 ; c1ccc(-c2ccc(N(c3ccc4c(c3)sc3ccccc34)c3ccc4ccc5cc6oc7ccccc7c6cc5c4c3)cc2)cc1 ; c1ccc(-c2ccc(N(c3ccc4ccc5cc6oc7ccccc7c6cc5c4c3)c3cccc4sc5ccccc5c34)cc2)cc1] 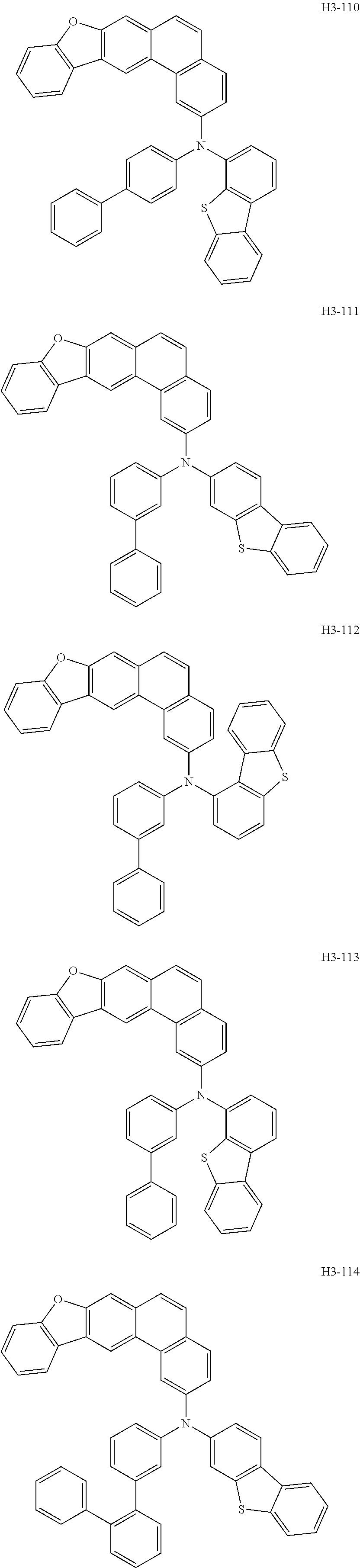 [SMILES: c1ccc(-c2ccc(N(c3ccc4ccc5cc6oc7ccccc7c6cc5c4c3)c3cccc4c3sc3ccccc34)cc2)cc1 ; c1ccc(-c2cccc(N(c3ccc4c(c3)sc3ccccc34)c3ccc4ccc5cc6oc7ccccc7c6cc5c4c3)c2)cc1 ; c1ccc(-c2cccc(N(c3ccc4ccc5cc6oc7ccccc7c6cc5c4c3)c3cccc4sc5ccccc5c34)c2)cc1 ; c1ccc(-c2cccc(N(c3ccc4ccc5cc6oc7ccccc7c6cc5c4c3)c3cccc4c3sc3ccccc34)c2)cc1 ; c1ccc(-c2ccccc2-c2cccc(N(c3ccc4c(c3)sc3ccccc34)c3ccc4ccc5cc6oc7ccccc7c6cc5c4c3)c2)cc1] 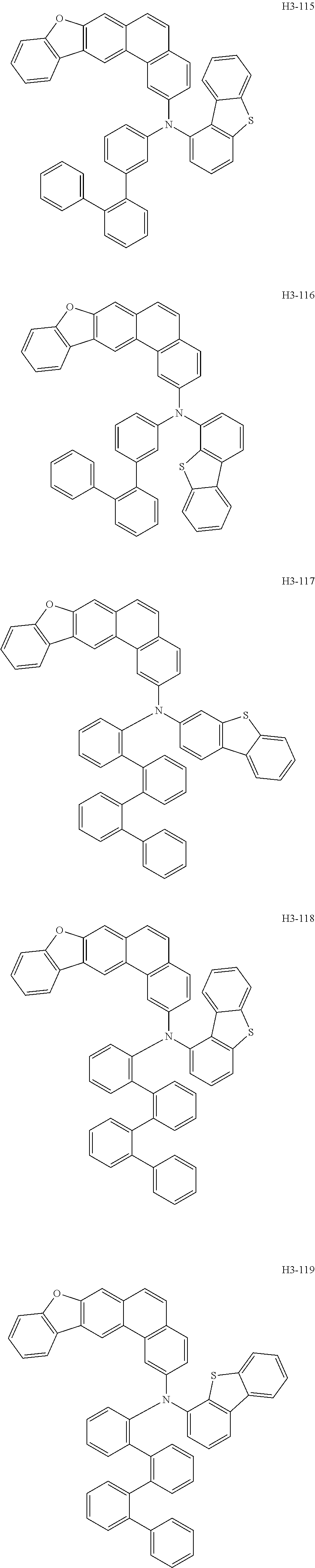 [SMILES: c1ccc(-c2ccccc2-c2cccc(N(c3ccc4ccc5cc6oc7ccccc7c6cc5c4c3)c3cccc4sc5ccccc5c34)c2)cc1 ; c1ccc(-c2ccccc2-c2cccc(N(c3ccc4ccc5cc6oc7ccccc7c6cc5c4c3)c3cccc4c3sc3ccccc34)c2)cc1 ; c1ccc(-c2ccccc2-c2ccccc2-c2ccccc2N(c2ccc3c(c2)sc2ccccc23)c2ccc3ccc4cc5oc6ccccc6c5cc4c3c2)cc1 ; c1ccc(-c2ccccc2-c2ccccc2-c2ccccc2N(c2ccc3ccc4cc5oc6ccccc6c5cc4c3c2)c2cccc3sc4ccccc4c23)cc1 ; c1ccc(-c2ccccc2-c2ccccc2-c2ccccc2N(c2ccc3ccc4cc5oc6ccccc6c5cc4c3c2)c2cccc3c2sc2ccccc23)cc1] 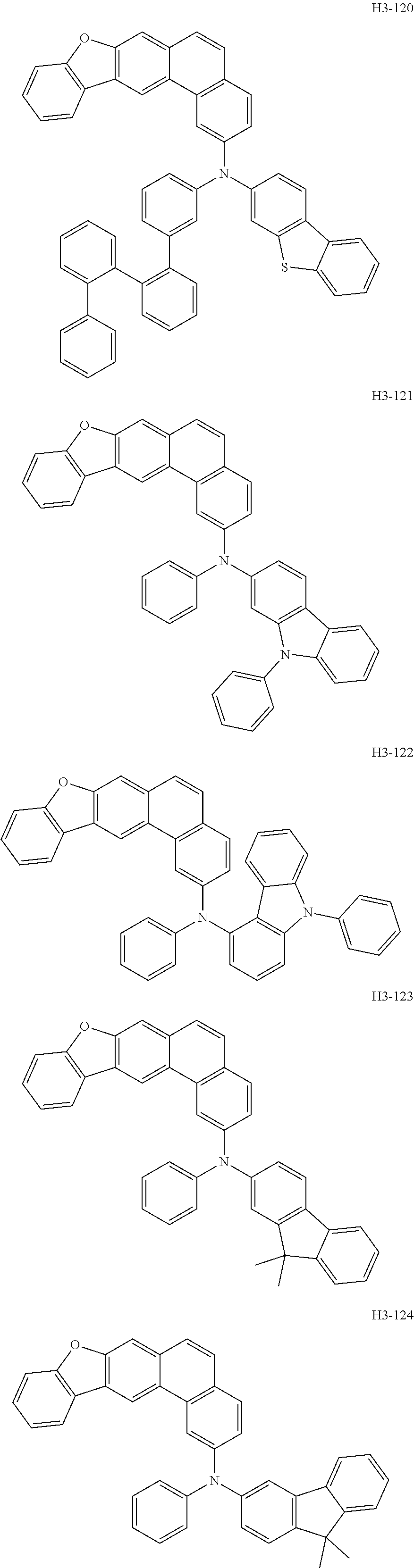 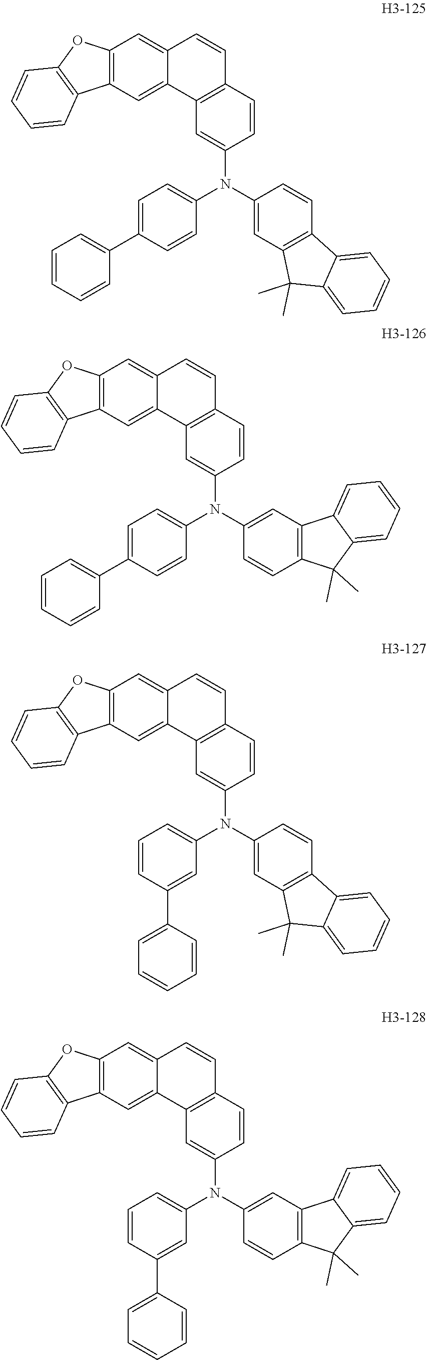 [SMILES: CC1(C)c2ccccc2-c2ccc(N(c3ccc(-c4ccccc4)cc3)c3ccc4ccc5cc6oc7ccccc7c6cc5c4c3)cc21 ; CC1(C)c2ccccc2-c2cc(N(c3ccc(-c4ccccc4)cc3)c3ccc4ccc5cc6oc7ccccc7c6cc5c4c3)ccc21 ; CC1(C)c2ccccc2-c2ccc(N(c3cccc(-c4ccccc4)c3)c3ccc4ccc5cc6oc7ccccc7c6cc5c4c3)cc21 ; CC1(C)c2ccccc2-c2cc(N(c3cccc(-c4ccccc4)c3)c3ccc4ccc5cc6oc7ccccc7c6cc5c4c3)ccc21] 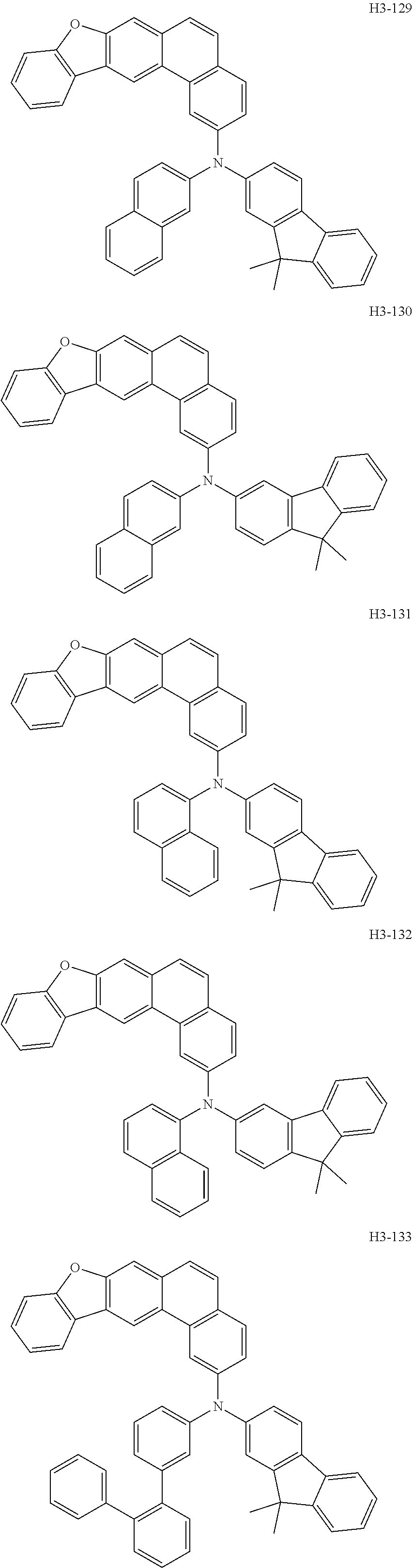 [SMILES: CC1(C)c2ccccc2-c2ccc(N(c3ccc4ccccc4c3)c3ccc4ccc5cc6oc7ccccc7c6cc5c4c3)cc21 ; CC1(C)c2ccccc2-c2cc(N(c3ccc4ccccc4c3)c3ccc4ccc5cc6oc7ccccc7c6cc5c4c3)ccc21 ; CC1(C)c2ccccc2-c2ccc(N(c3ccc4ccc5cc6oc7ccccc7c6cc5c4c3)c3cccc4ccccc34)cc21 ; CC1(C)c2ccccc2-c2cc(N(c3ccc4ccc5cc6oc7ccccc7c6cc5c4c3)c3cccc4ccccc34)ccc21 ; CC1(C)c2ccccc2-c2ccc(N(c3cccc(-c4ccccc4-c4ccccc4)c3)c3ccc4ccc5cc6oc7ccccc7c6cc5c4c3)cc21] 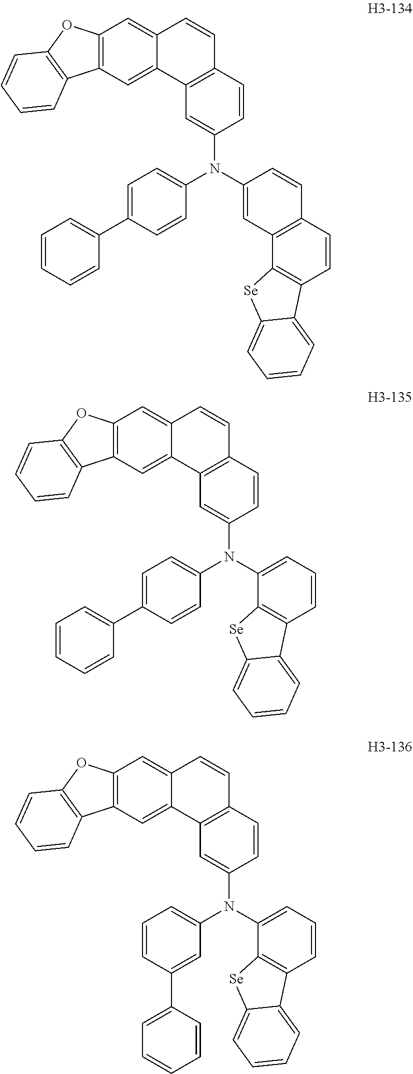 [SMILES: c1ccc(-c2ccc(N(c3ccc4ccc5cc6oc7ccccc7c6cc5c4c3)c3ccc4ccc5c6ccccc6[se]c5c4c3)cc2)cc1 ; c1ccc(-c2ccc(N(c3ccc4ccc5cc6oc7ccccc7c6cc5c4c3)c3cccc4c3[se]c3ccccc34)cc2)cc1 ; c1ccc(-c2cccc(N(c3ccc4ccc5cc6oc7ccccc7c6cc5c4c3)c3cccc4c3[se]c3ccccc34)c2)cc1] 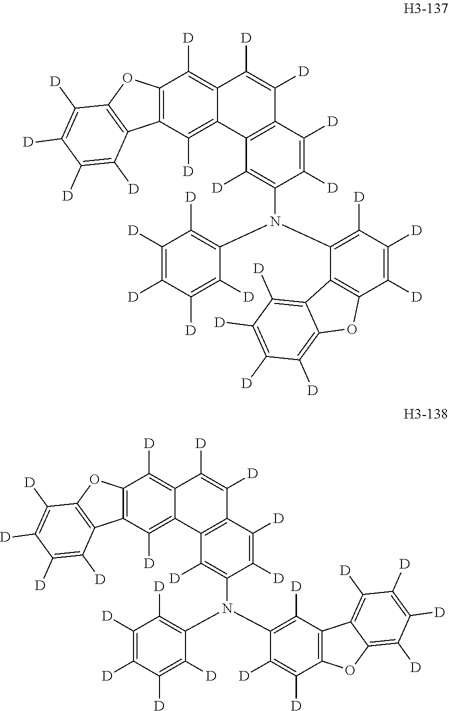 [SMILES: [2H]c1c([2H])c([2H])c(N(c2c([2H])c([2H])c3c([2H])c([2H])c4c([2H])c5oc6c([2H])c([2H])c([2H])c([2H])c6c5c([2H])c4c3c2[2H])c2c([2H])c([2H])c([2H])c3oc4c([2H])c([2H])c([2H])c([2H])c4c23)c([2H])c1[2H] ; [2H]c1c([2H])c([2H])c(N(c2c([2H])c([2H])c3oc4c([2H])c([2H])c([2H])c([2H])c4c3c2[2H])c2c([2H])c([2H])c3c([2H])c([2H])c4c([2H])c5oc6c([2H])c([2H])c([2H])c([2H])c6c5c([2H])c4c3c2[2H])c([2H])c1[2H]] 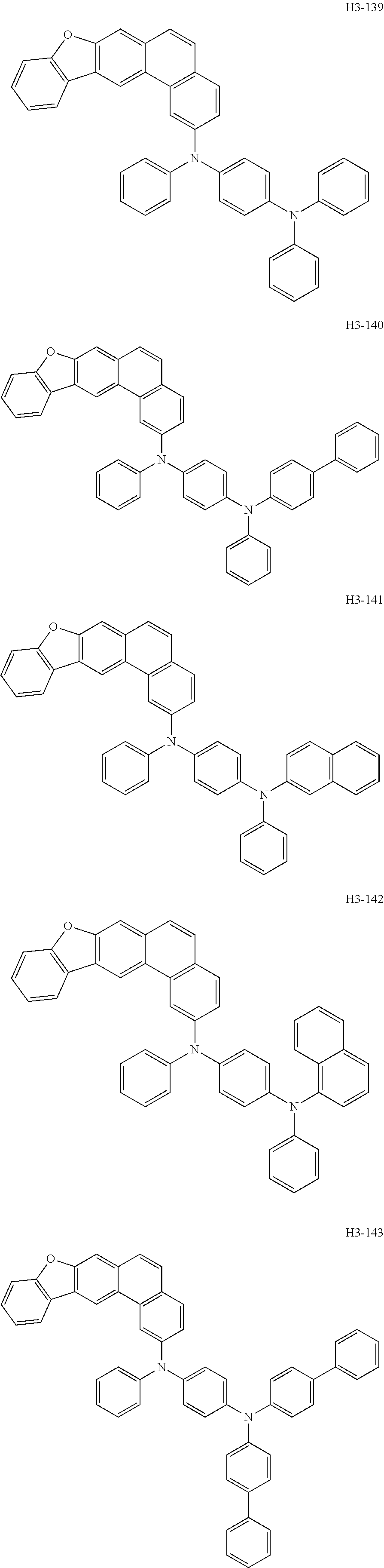 [SMILES: c1ccc(N(c2ccccc2)c2ccc(N(c3ccccc3)c3ccc4ccc5cc6oc7ccccc7c6cc5c4c3)cc2)cc1 ; c1ccc(-c2ccc(N(c3ccccc3)c3ccc(N(c4ccccc4)c4ccc5ccc6cc7oc8ccccc8c7cc6c5c4)cc3)cc2)cc1 ; c1ccc(N(c2ccc(N(c3ccccc3)c3ccc4ccc5cc6oc7ccccc7c6cc5c4c3)cc2)c2ccc3ccccc3c2)cc1 ; c1ccc(N(c2ccc(N(c3ccccc3)c3cccc4ccccc34)cc2)c2ccc3ccc4cc5oc6ccccc6c5cc4c3c2)cc1 ; c1ccc(-c2ccc(N(c3ccc(-c4ccccc4)cc3)c3ccc(N(c4ccccc4)c4ccc5ccc6cc7oc8ccccc8c7cc6c5c4)cc3)cc2)cc1] 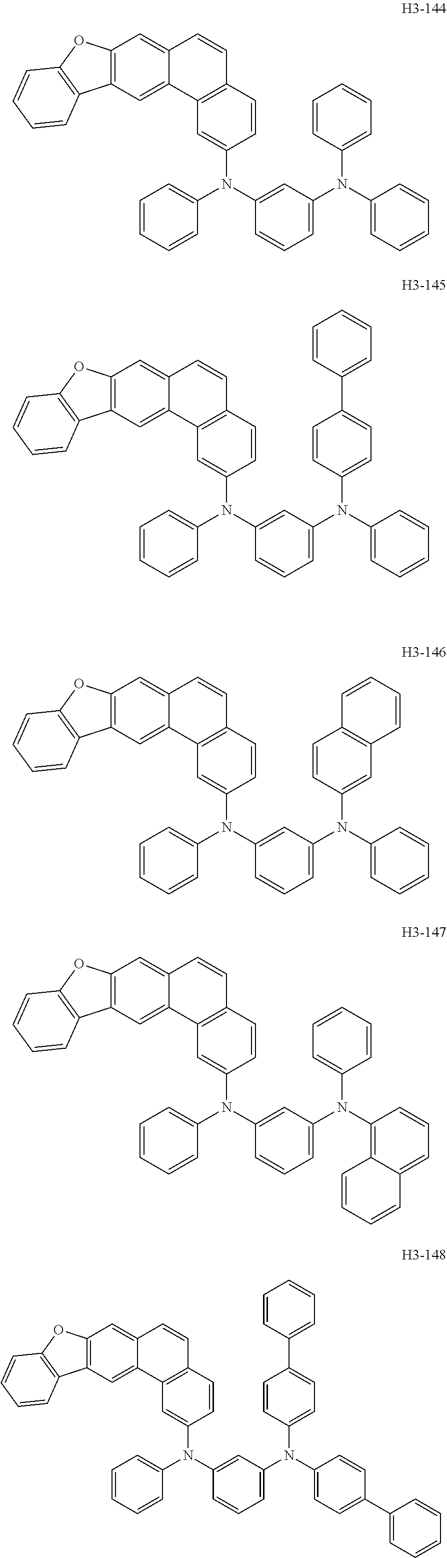 [SMILES: c1ccc(N(c2ccccc2)c2cccc(N(c3ccccc3)c3ccc4ccc5cc6oc7ccccc7c6cc5c4c3)c2)cc1 ; c1ccc(-c2ccc(N(c3ccccc3)c3cccc(N(c4ccccc4)c4ccc5ccc6cc7oc8ccccc8c7cc6c5c4)c3)cc2)cc1 ; c1ccc(N(c2cccc(N(c3ccccc3)c3ccc4ccc5cc6oc7ccccc7c6cc5c4c3)c2)c2ccc3ccccc3c2)cc1 ; c1ccc(N(c2cccc(N(c3ccccc3)c3cccc4ccccc34)c2)c2ccc3ccc4cc5oc6ccccc6c5cc4c3c2)cc1 ; c1ccc(-c2ccc(N(c3ccc(-c4ccccc4)cc3)c3cccc(N(c4ccccc4)c4ccc5ccc6cc7oc8ccccc8c7cc6c5c4)c3)cc2)cc1] 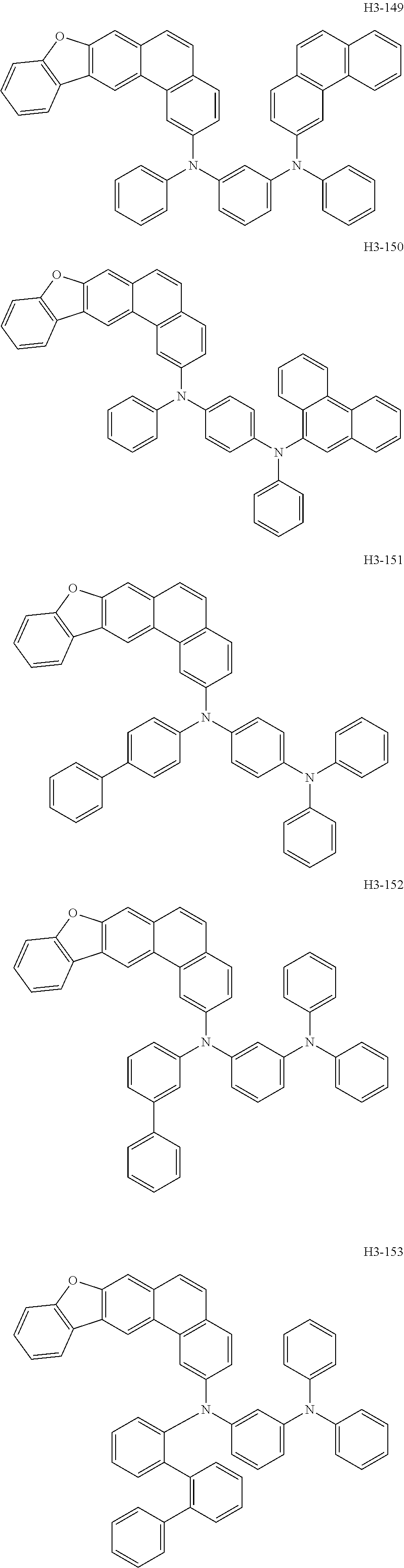 [SMILES: c1ccc(N(c2cccc(N(c3ccccc3)c3ccc4ccc5cc6oc7ccccc7c6cc5c4c3)c2)c2ccc3ccc4ccccc4c3c2)cc1 ; c1ccc(N(c2ccc(N(c3ccccc3)c3cc4ccccc4c4ccccc34)cc2)c2ccc3ccc4cc5oc6ccccc6c5cc4c3c2)cc1 ; c1ccc(-c2ccc(N(c3ccc(N(c4ccccc4)c4ccccc4)cc3)c3ccc4ccc5cc6oc7ccccc7c6cc5c4c3)cc2)cc1 ; c1ccc(-c2cccc(N(c3cccc(N(c4ccccc4)c4ccccc4)c3)c3ccc4ccc5cc6oc7ccccc7c6cc5c4c3)c2)cc1 ; c1ccc(-c2ccccc2-c2ccccc2N(c2cccc(N(c3ccccc3)c3ccccc3)c2)c2ccc3ccc4cc5oc6ccccc6c5cc4c3c2)cc1] 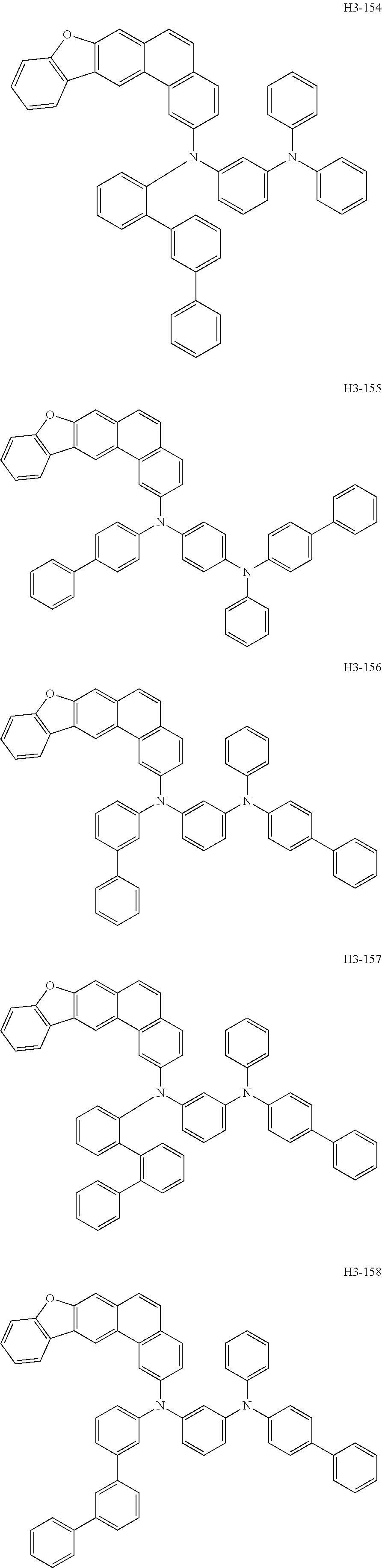 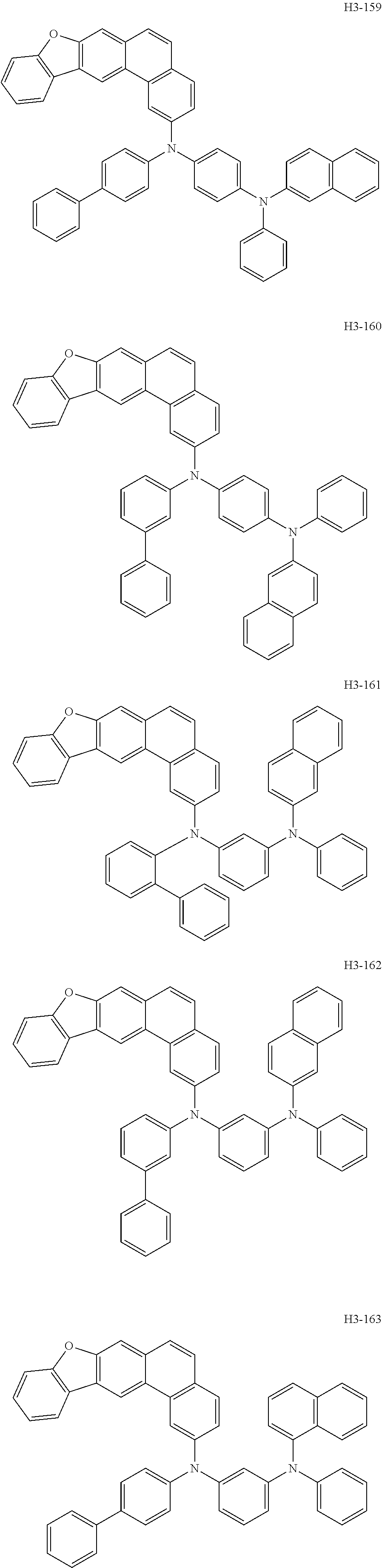 [SMILES: c1ccc(-c2ccc(N(c3ccc(N(c4ccccc4)c4ccc5ccccc5c4)cc3)c3ccc4ccc5cc6oc7ccccc7c6cc5c4c3)cc2)cc1 ; c1ccc(-c2cccc(N(c3ccc(N(c4ccccc4)c4ccc5ccccc5c4)cc3)c3ccc4ccc5cc6oc7ccccc7c6cc5c4c3)c2)cc1 ; c1ccc(-c2ccccc2N(c2cccc(N(c3ccccc3)c3ccc4ccccc4c3)c2)c2ccc3ccc4cc5oc6ccccc6c5cc4c3c2)cc1 ; c1ccc(-c2cccc(N(c3cccc(N(c4ccccc4)c4ccc5ccccc5c4)c3)c3ccc4ccc5cc6oc7ccccc7c6cc5c4c3)c2)cc1 ; c1ccc(-c2ccc(N(c3cccc(N(c4ccccc4)c4cccc5ccccc45)c3)c3ccc4ccc5cc6oc7ccccc7c6cc5c4c3)cc2)cc1] 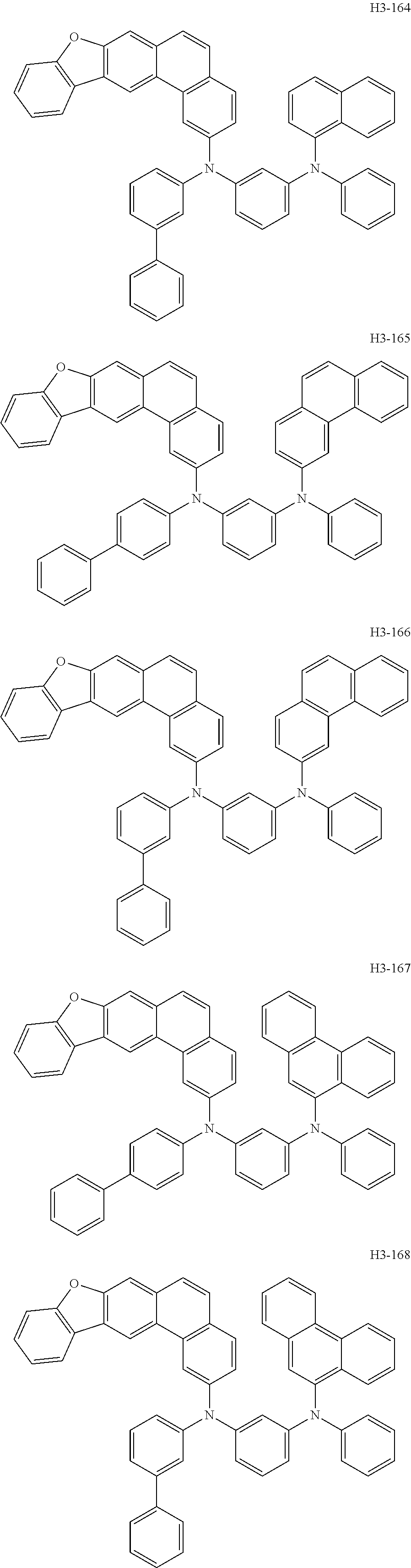 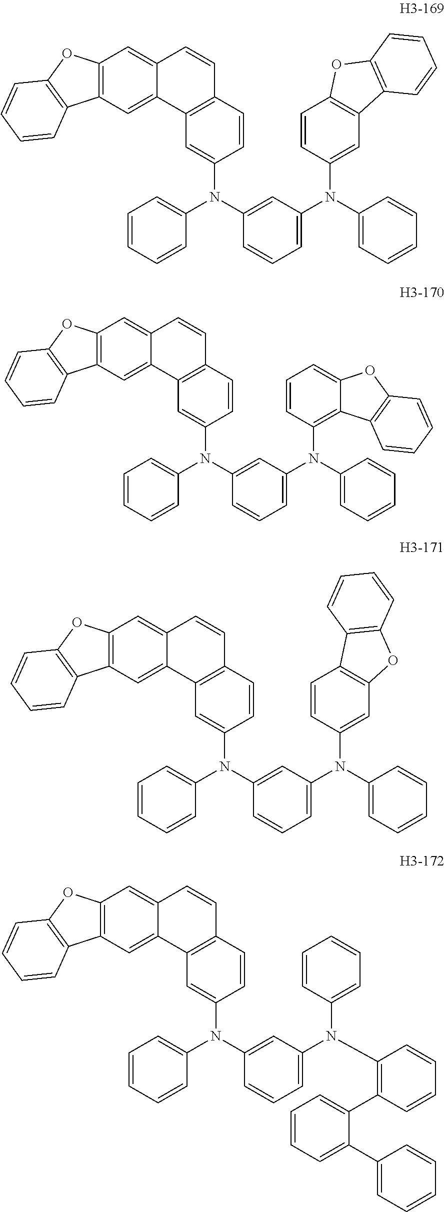 [SMILES: c1ccc(N(c2cccc(N(c3ccccc3)c3ccc4oc5ccccc5c4c3)c2)c2ccc3ccc4cc5oc6ccccc6c5cc4c3c2)cc1 ; c1ccc(N(c2cccc(N(c3ccccc3)c3cccc4oc5ccccc5c34)c2)c2ccc3ccc4cc5oc6ccccc6c5cc4c3c2)cc1 ; c1ccc(N(c2cccc(N(c3ccccc3)c3ccc4ccc5cc6oc7ccccc7c6cc5c4c3)c2)c2ccc3c(c2)oc2ccccc23)cc1 ; c1ccc(-c2ccccc2-c2ccccc2N(c2ccccc2)c2cccc(N(c3ccccc3)c3ccc4ccc5cc6oc7ccccc7c6cc5c4c3)c2)cc1] 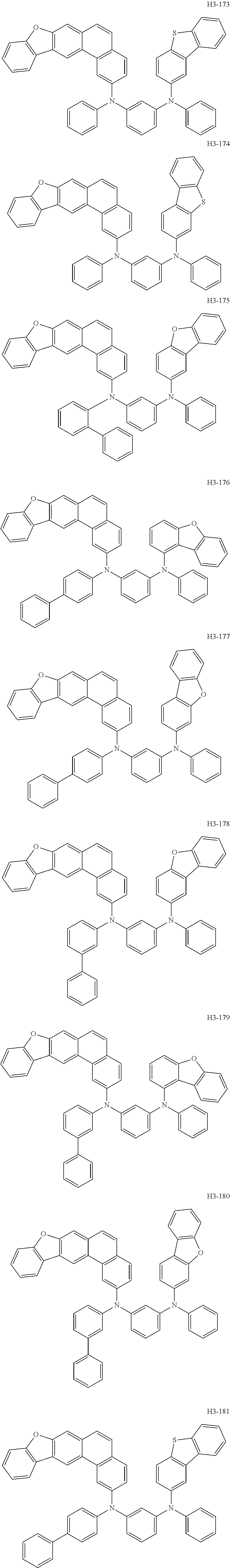 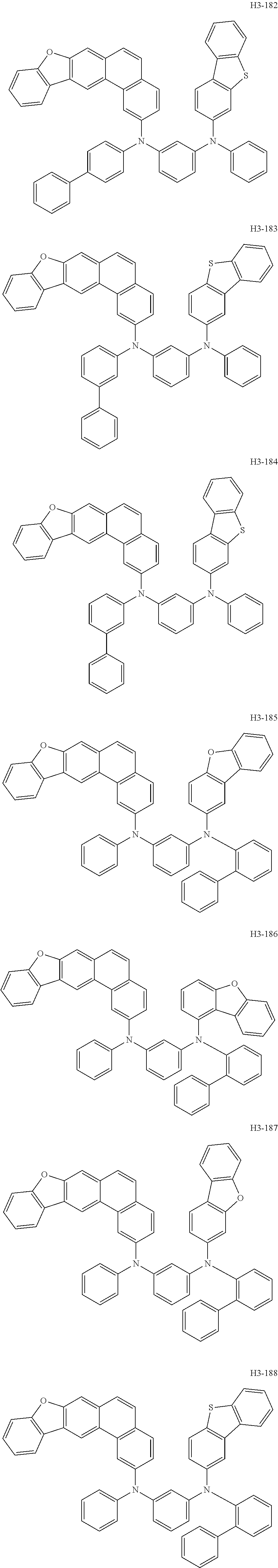 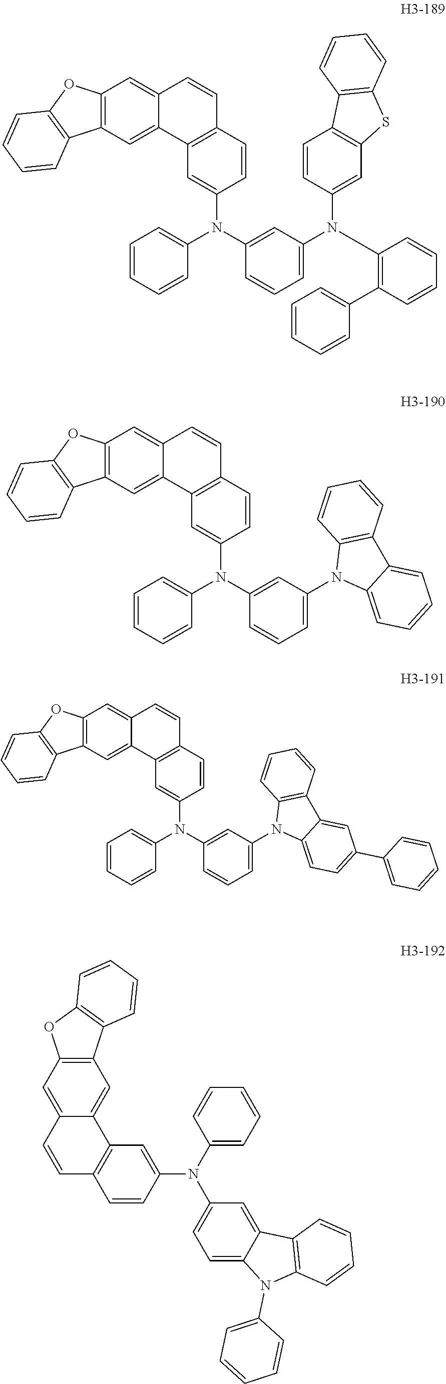 [SMILES: c1ccc(-c2ccccc2N(c2cccc(N(c3ccccc3)c3ccc4ccc5cc6oc7ccccc7c6cc5c4c3)c2)c2ccc3c(c2)sc2ccccc23)cc1 ; c1ccc(N(c2cccc(-n3c4ccccc4c4ccccc43)c2)c2ccc3ccc4cc5oc6ccccc6c5cc4c3c2)cc1 ; c1ccc(-c2ccc3c(c2)c2ccccc2n3-c2cccc(N(c3ccccc3)c3ccc4ccc5cc6oc7ccccc7c6cc5c4c3)c2)cc1 ; c1ccc(N(c2ccc3ccc4cc5oc6ccccc6c5cc4c3c2)c2ccc3c(c2)c2ccccc2n3-c2ccccc2)cc1] 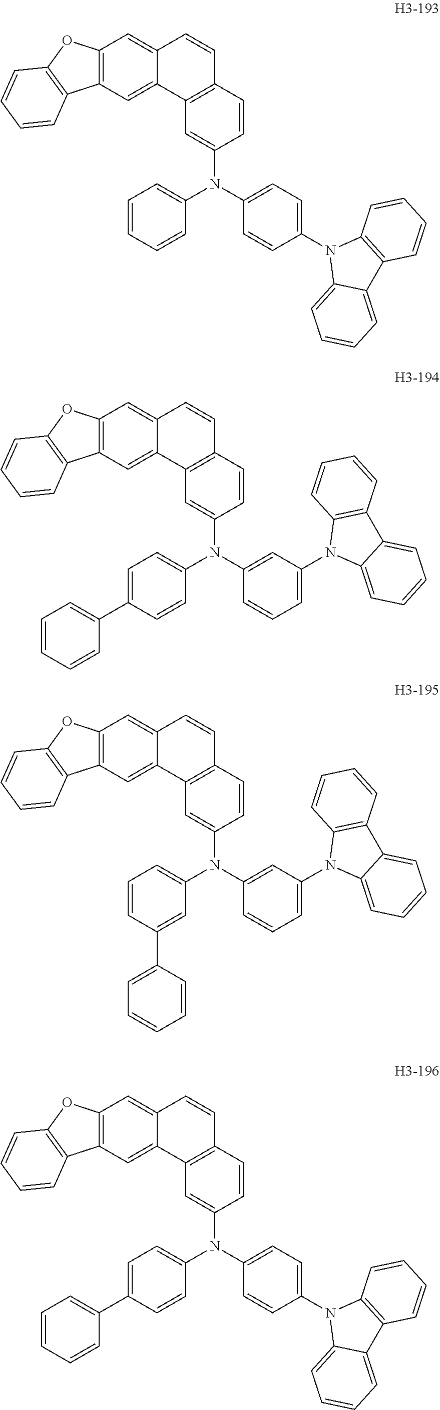 [SMILES: c1ccc(N(c2ccc(-n3c4ccccc4c4ccccc43)cc2)c2ccc3ccc4cc5oc6ccccc6c5cc4c3c2)cc1 ; c1ccc(-c2ccc(N(c3cccc(-n4c5ccccc5c5ccccc54)c3)c3ccc4ccc5cc6oc7ccccc7c6cc5c4c3)cc2)cc1 ; c1ccc(-c2cccc(N(c3cccc(-n4c5ccccc5c5ccccc54)c3)c3ccc4ccc5cc6oc7ccccc7c6cc5c4c3)c2)cc1 ; c1ccc(-c2ccc(N(c3ccc(-n4c5ccccc5c5ccccc54)cc3)c3ccc4ccc5cc6oc7ccccc7c6cc5c4c3)cc2)cc1] 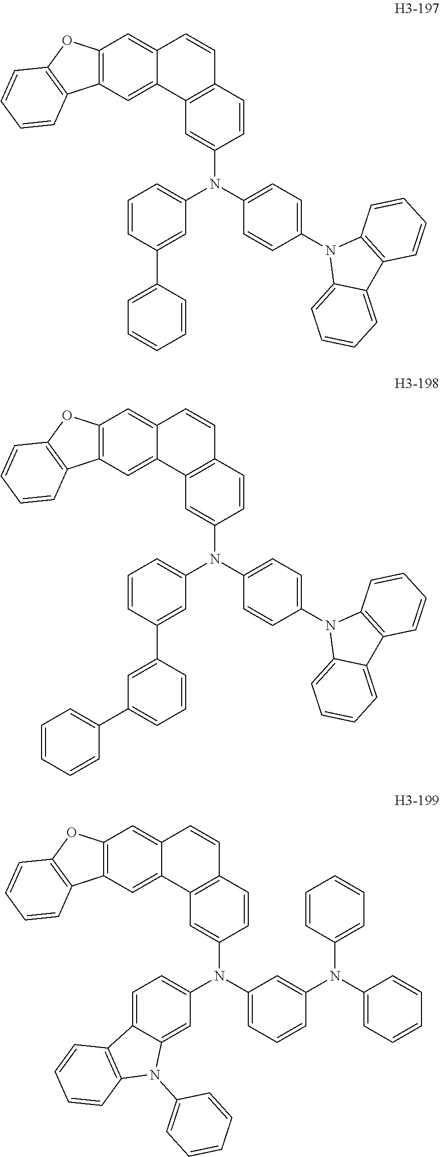 [SMILES: c1ccc(-c2cccc(N(c3ccc(-n4c5ccccc5c5ccccc54)cc3)c3ccc4ccc5cc6oc7ccccc7c6cc5c4c3)c2)cc1 ; c1ccc(-c2cccc(-c3cccc(N(c4ccc(-n5c6ccccc6c6ccccc65)cc4)c4ccc5ccc6cc7oc8ccccc8c7cc6c5c4)c3)c2)cc1 ; c1ccc(N(c2ccccc2)c2cccc(N(c3ccc4ccc5cc6oc7ccccc7c6cc5c4c3)c3ccc4c5ccccc5n(-c5ccccc5)c4c3)c2)cc1] 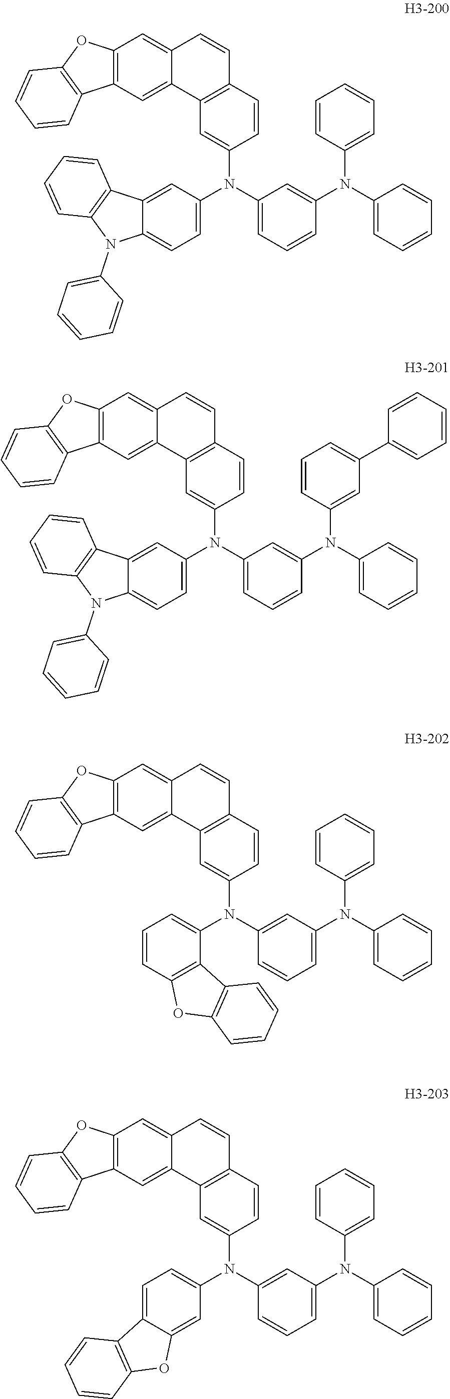 [SMILES: c1ccc(N(c2ccccc2)c2cccc(N(c3ccc4ccc5cc6oc7ccccc7c6cc5c4c3)c3ccc4c(c3)c3ccccc3n4-c3ccccc3)c2)cc1 ; c1ccc(-c2cccc(N(c3ccccc3)c3cccc(N(c4ccc5ccc6cc7oc8ccccc8c7cc6c5c4)c4ccc5c(c4)c4ccccc4n5-c4ccccc4)c3)c2)cc1 ; c1ccc(N(c2ccccc2)c2cccc(N(c3ccc4ccc5cc6oc7ccccc7c6cc5c4c3)c3cccc4oc5ccccc5c34)c2)cc1 ; c1ccc(N(c2ccccc2)c2cccc(N(c3ccc4c(c3)oc3ccccc34)c3ccc4ccc5cc6oc7ccccc7c6cc5c4c3)c2)cc1] 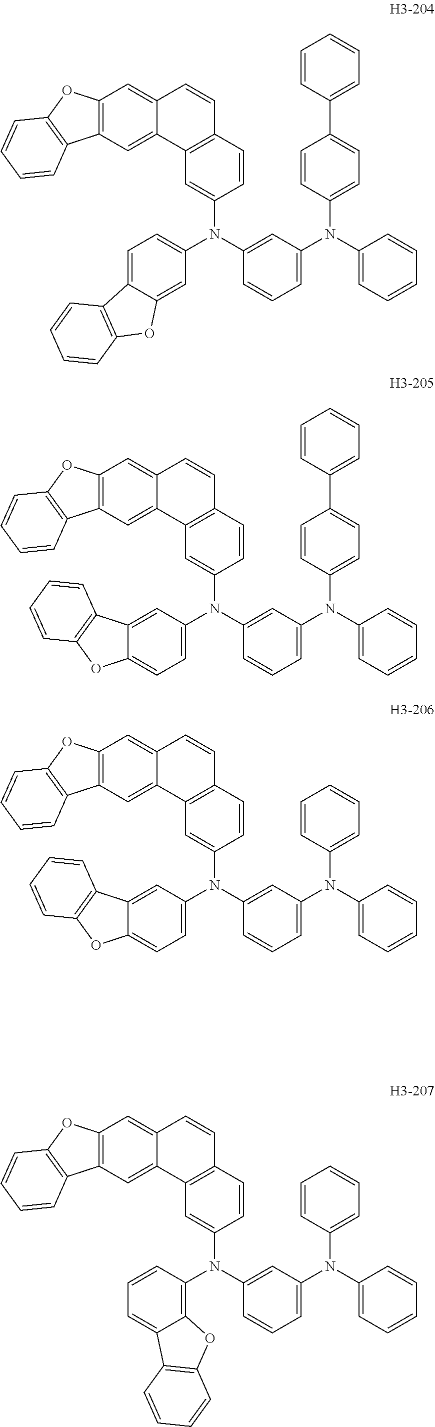 [SMILES: c1ccc(-c2ccc(N(c3ccccc3)c3cccc(N(c4ccc5c(c4)oc4ccccc45)c4ccc5ccc6cc7oc8ccccc8c7cc6c5c4)c3)cc2)cc1 ; c1ccc(-c2ccc(N(c3ccccc3)c3cccc(N(c4ccc5ccc6cc7oc8ccccc8c7cc6c5c4)c4ccc5oc6ccccc6c5c4)c3)cc2)cc1 ; c1ccc(N(c2ccccc2)c2cccc(N(c3ccc4ccc5cc6oc7ccccc7c6cc5c4c3)c3ccc4oc5ccccc5c4c3)c2)cc1 ; c1ccc(N(c2ccccc2)c2cccc(N(c3ccc4ccc5cc6oc7ccccc7c6cc5c4c3)c3cccc4c3oc3ccccc34)c2)cc1] 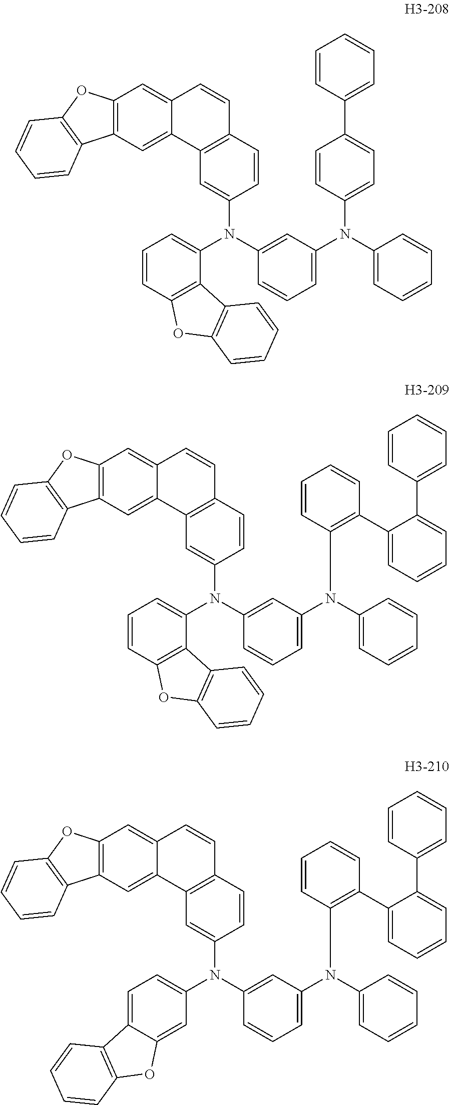 [SMILES: c1ccc(-c2ccc(N(c3ccccc3)c3cccc(N(c4ccc5ccc6cc7oc8ccccc8c7cc6c5c4)c4cccc5oc6ccccc6c45)c3)cc2)cc1 ; c1ccc(-c2ccccc2-c2ccccc2N(c2ccccc2)c2cccc(N(c3ccc4ccc5cc6oc7ccccc7c6cc5c4c3)c3cccc4oc5ccccc5c34)c2)cc1 ; c1ccc(-c2ccccc2-c2ccccc2N(c2ccccc2)c2cccc(N(c3ccc4c(c3)oc3ccccc34)c3ccc4ccc5cc6oc7ccccc7c6cc5c4c3)c2)cc1] 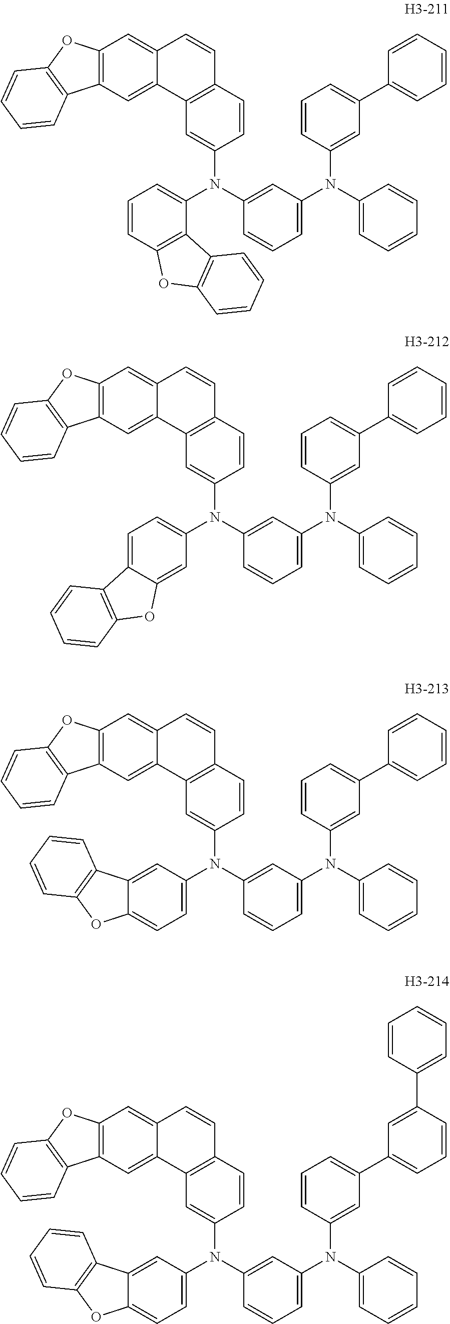 [SMILES: c1ccc(-c2cccc(N(c3ccccc3)c3cccc(N(c4ccc5ccc6cc7oc8ccccc8c7cc6c5c4)c4cccc5oc6ccccc6c45)c3)c2)cc1 ; c1ccc(-c2cccc(N(c3ccccc3)c3cccc(N(c4ccc5c(c4)oc4ccccc45)c4ccc5ccc6cc7oc8ccccc8c7cc6c5c4)c3)c2)cc1 ; c1ccc(-c2cccc(N(c3ccccc3)c3cccc(N(c4ccc5ccc6cc7oc8ccccc8c7cc6c5c4)c4ccc5oc6ccccc6c5c4)c3)c2)cc1 ; c1ccc(-c2cccc(-c3cccc(N(c4ccccc4)c4cccc(N(c5ccc6ccc7cc8oc9ccccc9c8cc7c6c5)c5ccc6oc7ccccc7c6c5)c4)c3)c2)cc1] 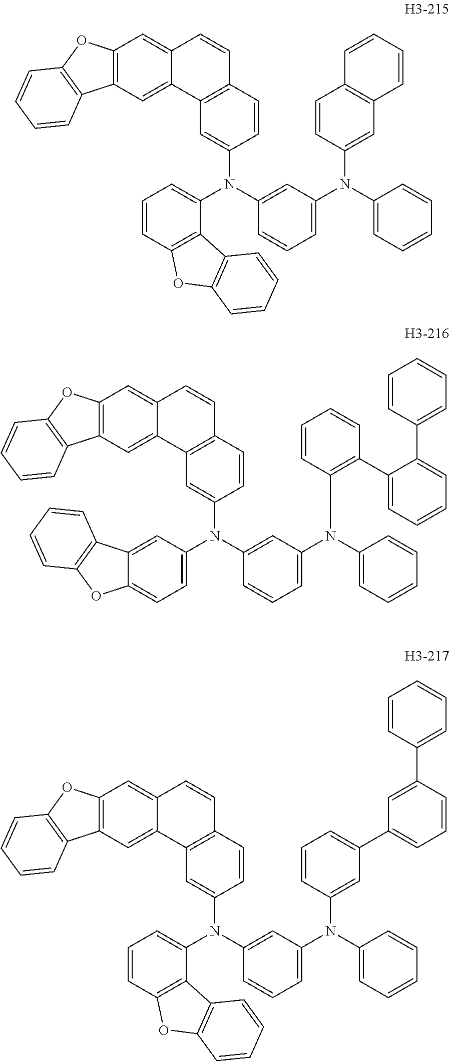 [SMILES: c1ccc(N(c2cccc(N(c3ccc4ccc5cc6oc7ccccc7c6cc5c4c3)c3cccc4oc5ccccc5c34)c2)c2ccc3ccccc3c2)cc1 ; c1ccc(-c2ccccc2-c2ccccc2N(c2ccccc2)c2cccc(N(c3ccc4ccc5cc6oc7ccccc7c6cc5c4c3)c3ccc4oc5ccccc5c4c3)c2)cc1 ; c1ccc(-c2cccc(-c3cccc(N(c4ccccc4)c4cccc(N(c5ccc6ccc7cc8oc9ccccc9c8cc7c6c5)c5cccc6oc7ccccc7c56)c4)c3)c2)cc1] 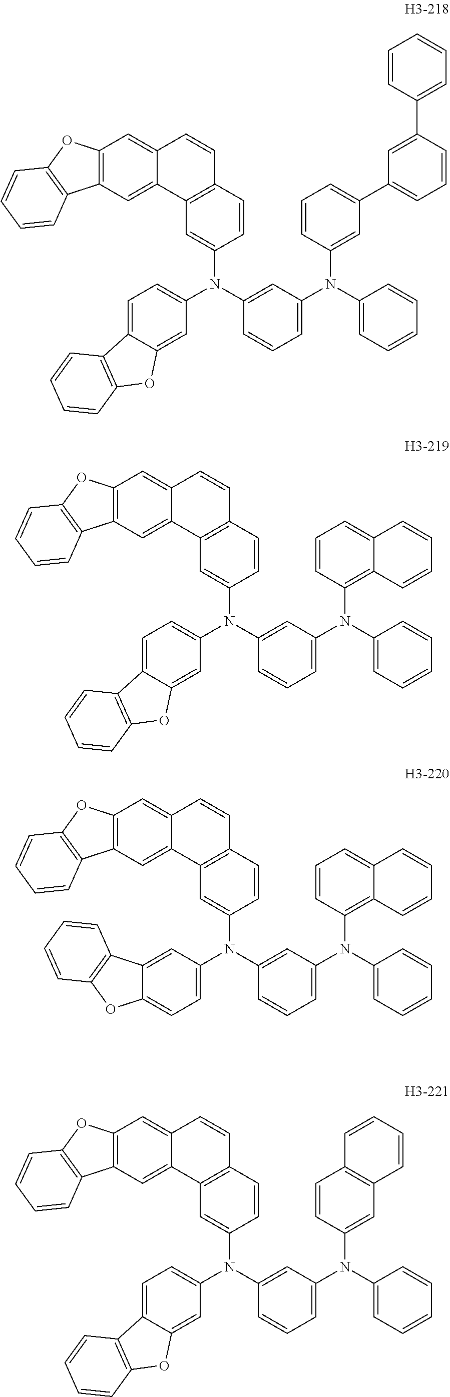 [SMILES: c1ccc(-c2cccc(-c3cccc(N(c4ccccc4)c4cccc(N(c5ccc6c(c5)oc5ccccc56)c5ccc6ccc7cc8oc9ccccc9c8cc7c6c5)c4)c3)c2)cc1 ; c1ccc(N(c2cccc(N(c3ccc4c(c3)oc3ccccc34)c3ccc4ccc5cc6oc7ccccc7c6cc5c4c3)c2)c2cccc3ccccc23)cc1 ; c1ccc(N(c2cccc(N(c3ccc4ccc5cc6oc7ccccc7c6cc5c4c3)c3ccc4oc5ccccc5c4c3)c2)c2cccc3ccccc23)cc1 ; c1ccc(N(c2cccc(N(c3ccc4c(c3)oc3ccccc34)c3ccc4ccc5cc6oc7ccccc7c6cc5c4c3)c2)c2ccc3ccccc3c2)cc1] 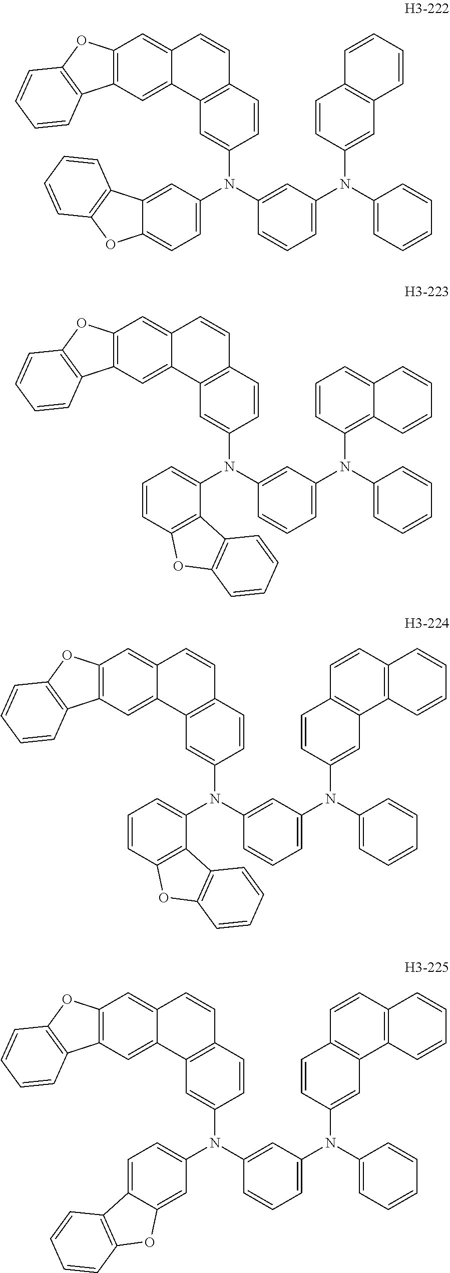 [SMILES: c1ccc(N(c2cccc(N(c3ccc4ccc5cc6oc7ccccc7c6cc5c4c3)c3ccc4oc5ccccc5c4c3)c2)c2ccc3ccccc3c2)cc1 ; c1ccc(N(c2cccc(N(c3ccc4ccc5cc6oc7ccccc7c6cc5c4c3)c3cccc4oc5ccccc5c34)c2)c2cccc3ccccc23)cc1 ; c1ccc(N(c2cccc(N(c3ccc4ccc5cc6oc7ccccc7c6cc5c4c3)c3cccc4oc5ccccc5c34)c2)c2ccc3ccc4ccccc4c3c2)cc1 ; c1ccc(N(c2cccc(N(c3ccc4c(c3)oc3ccccc34)c3ccc4ccc5cc6oc7ccccc7c6cc5c4c3)c2)c2ccc3ccc4ccccc4c3c2)cc1] 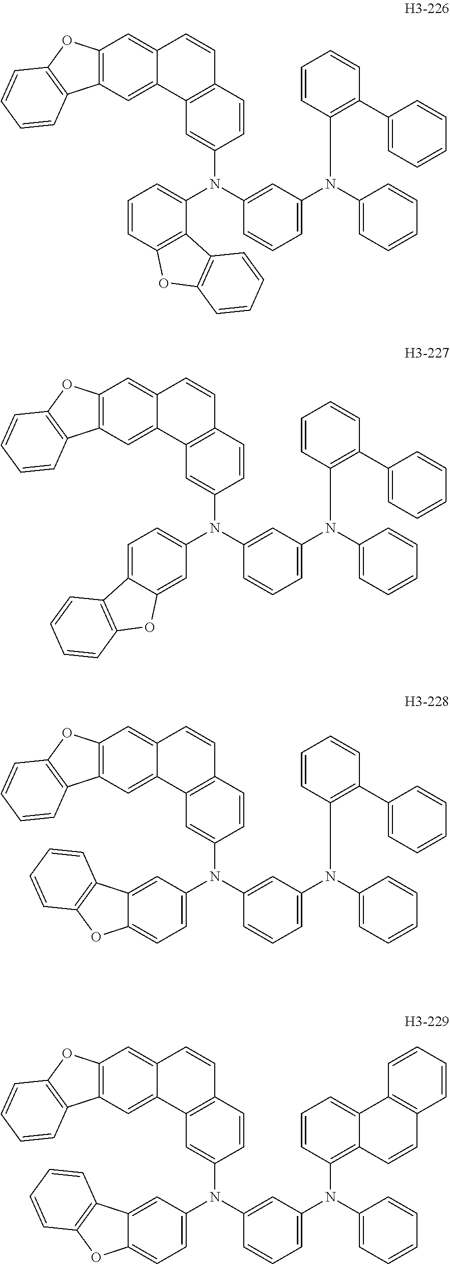 [SMILES: c1ccc(-c2ccccc2N(c2ccccc2)c2cccc(N(c3ccc4ccc5cc6oc7ccccc7c6cc5c4c3)c3cccc4oc5ccccc5c34)c2)cc1 ; c1ccc(-c2ccccc2N(c2ccccc2)c2cccc(N(c3ccc4c(c3)oc3ccccc34)c3ccc4ccc5cc6oc7ccccc7c6cc5c4c3)c2)cc1 ; c1ccc(-c2ccccc2N(c2ccccc2)c2cccc(N(c3ccc4ccc5cc6oc7ccccc7c6cc5c4c3)c3ccc4oc5ccccc5c4c3)c2)cc1 ; c1ccc(N(c2cccc(N(c3ccc4ccc5cc6oc7ccccc7c6cc5c4c3)c3ccc4oc5ccccc5c4c3)c2)c2cccc3c2ccc2ccccc23)cc1] 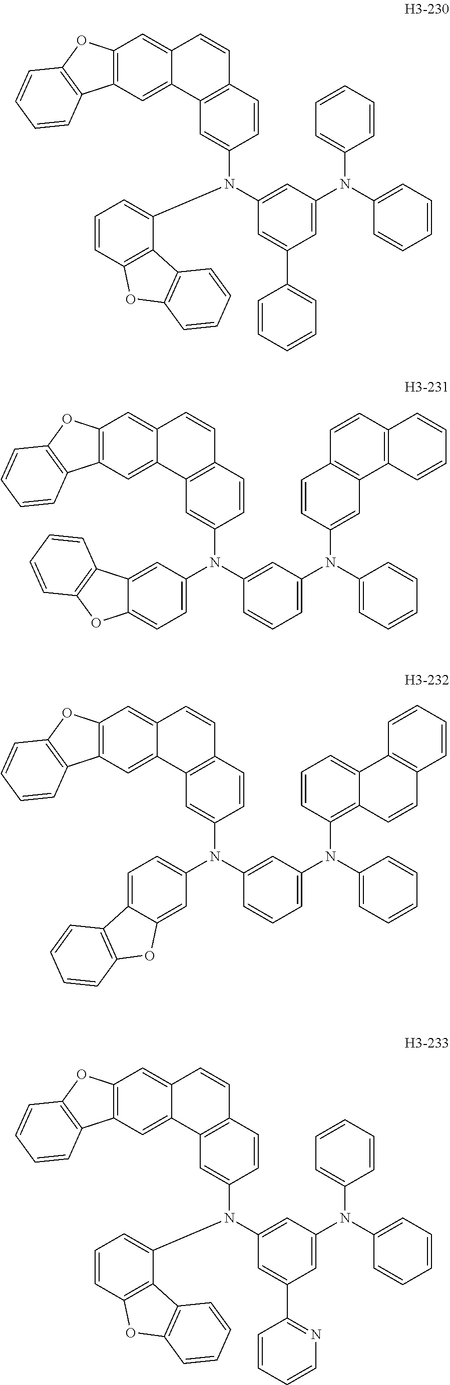 [SMILES: c1ccc(-c2cc(N(c3ccccc3)c3ccccc3)cc(N(c3ccc4ccc5cc6oc7ccccc7c6cc5c4c3)c3cccc4oc5ccccc5c34)c2)cc1 ; c1ccc(N(c2cccc(N(c3ccc4ccc5cc6oc7ccccc7c6cc5c4c3)c3ccc4oc5ccccc5c4c3)c2)c2ccc3ccc4ccccc4c3c2)cc1 ; c1ccc(N(c2cccc(N(c3ccc4c(c3)oc3ccccc34)c3ccc4ccc5cc6oc7ccccc7c6cc5c4c3)c2)c2cccc3c2ccc2ccccc23)cc1 ; c1ccc(N(c2ccccc2)c2cc(-c3ccccn3)cc(N(c3ccc4ccc5cc6oc7ccccc7c6cc5c4c3)c3cccc4oc5ccccc5c34)c2)cc1] 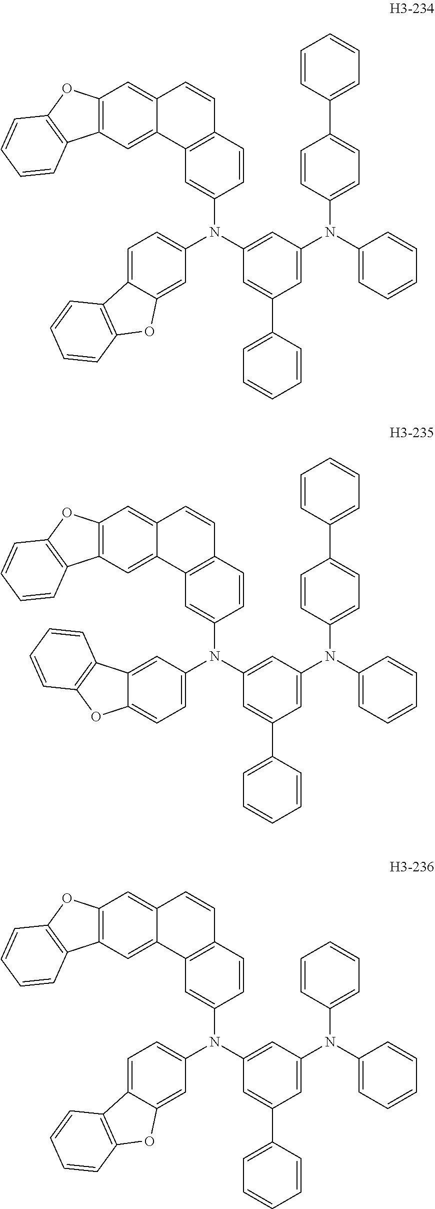 [SMILES: c1ccc(-c2ccc(N(c3ccccc3)c3cc(-c4ccccc4)cc(N(c4ccc5c(c4)oc4ccccc45)c4ccc5ccc6cc7oc8ccccc8c7cc6c5c4)c3)cc2)cc1 ; c1ccc(-c2ccc(N(c3ccccc3)c3cc(-c4ccccc4)cc(N(c4ccc5ccc6cc7oc8ccccc8c7cc6c5c4)c4ccc5oc6ccccc6c5c4)c3)cc2)cc1 ; c1ccc(-c2cc(N(c3ccccc3)c3ccccc3)cc(N(c3ccc4c(c3)oc3ccccc34)c3ccc4ccc5cc6oc7ccccc7c6cc5c4c3)c2)cc1] 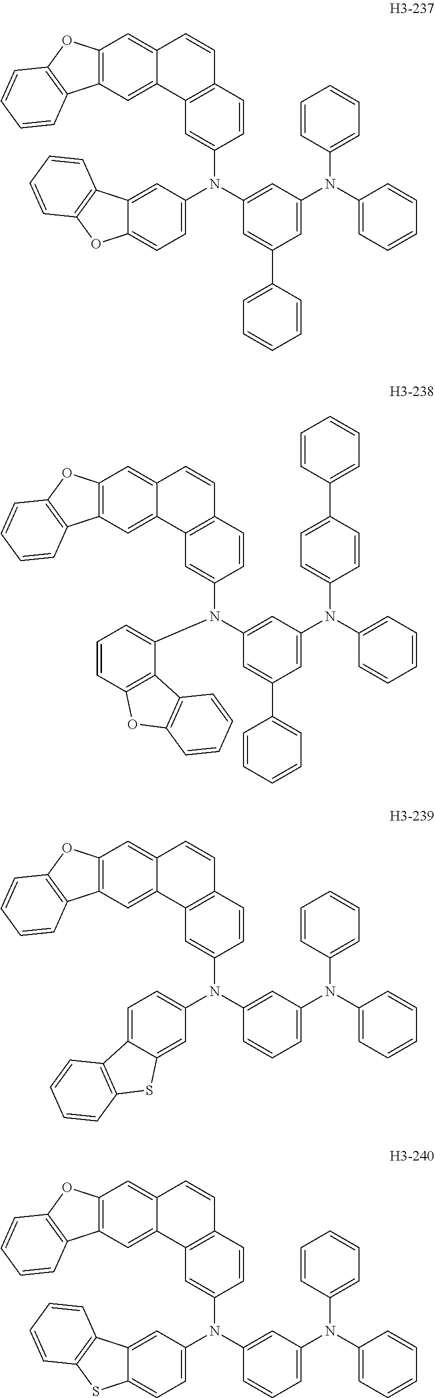 [SMILES: c1ccc(-c2cc(N(c3ccccc3)c3ccccc3)cc(N(c3ccc4ccc5cc6oc7ccccc7c6cc5c4c3)c3ccc4oc5ccccc5c4c3)c2)cc1 ; c1ccc(-c2ccc(N(c3ccccc3)c3cc(-c4ccccc4)cc(N(c4ccc5ccc6cc7oc8ccccc8c7cc6c5c4)c4cccc5oc6ccccc6c45)c3)cc2)cc1 ; c1ccc(N(c2ccccc2)c2cccc(N(c3ccc4c(c3)sc3ccccc34)c3ccc4ccc5cc6oc7ccccc7c6cc5c4c3)c2)cc1 ; c1ccc(N(c2ccccc2)c2cccc(N(c3ccc4ccc5cc6oc7ccccc7c6cc5c4c3)c3ccc4sc5ccccc5c4c3)c2)cc1] 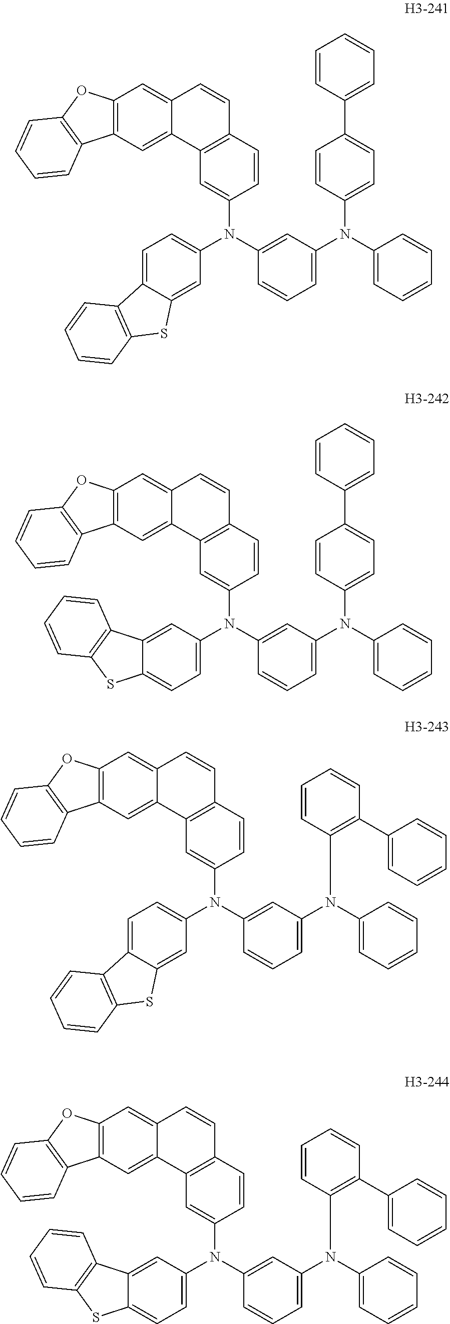 [SMILES: c1ccc(-c2ccc(N(c3ccccc3)c3cccc(N(c4ccc5c(c4)sc4ccccc45)c4ccc5ccc6cc7oc8ccccc8c7cc6c5c4)c3)cc2)cc1 ; c1ccc(-c2ccc(N(c3ccccc3)c3cccc(N(c4ccc5ccc6cc7oc8ccccc8c7cc6c5c4)c4ccc5sc6ccccc6c5c4)c3)cc2)cc1 ; c1ccc(-c2ccccc2N(c2ccccc2)c2cccc(N(c3ccc4c(c3)sc3ccccc34)c3ccc4ccc5cc6oc7ccccc7c6cc5c4c3)c2)cc1 ; c1ccc(-c2ccccc2N(c2ccccc2)c2cccc(N(c3ccc4ccc5cc6oc7ccccc7c6cc5c4c3)c3ccc4sc5ccccc5c4c3)c2)cc1] 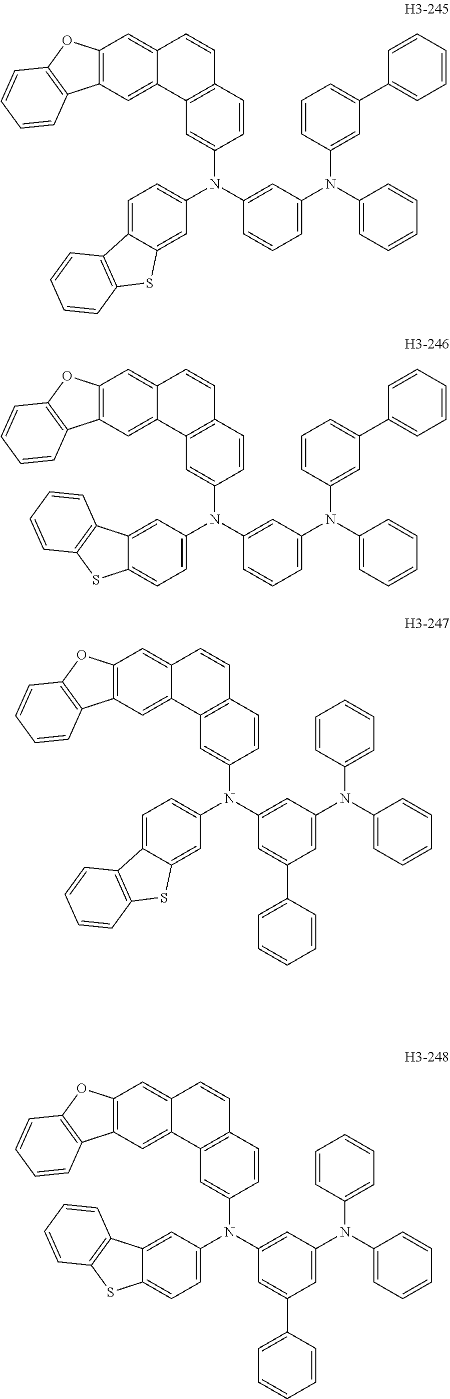 [SMILES: c1ccc(-c2cccc(N(c3ccccc3)c3cccc(N(c4ccc5c(c4)sc4ccccc45)c4ccc5ccc6cc7oc8ccccc8c7cc6c5c4)c3)c2)cc1 ; c1ccc(-c2cccc(N(c3ccccc3)c3cccc(N(c4ccc5ccc6cc7oc8ccccc8c7cc6c5c4)c4ccc5sc6ccccc6c5c4)c3)c2)cc1 ; c1ccc(-c2cc(N(c3ccccc3)c3ccccc3)cc(N(c3ccc4c(c3)sc3ccccc34)c3ccc4ccc5cc6oc7ccccc7c6cc5c4c3)c2)cc1 ; c1ccc(-c2cc(N(c3ccccc3)c3ccccc3)cc(N(c3ccc4ccc5cc6oc7ccccc7c6cc5c4c3)c3ccc4sc5ccccc5c4c3)c2)cc1] 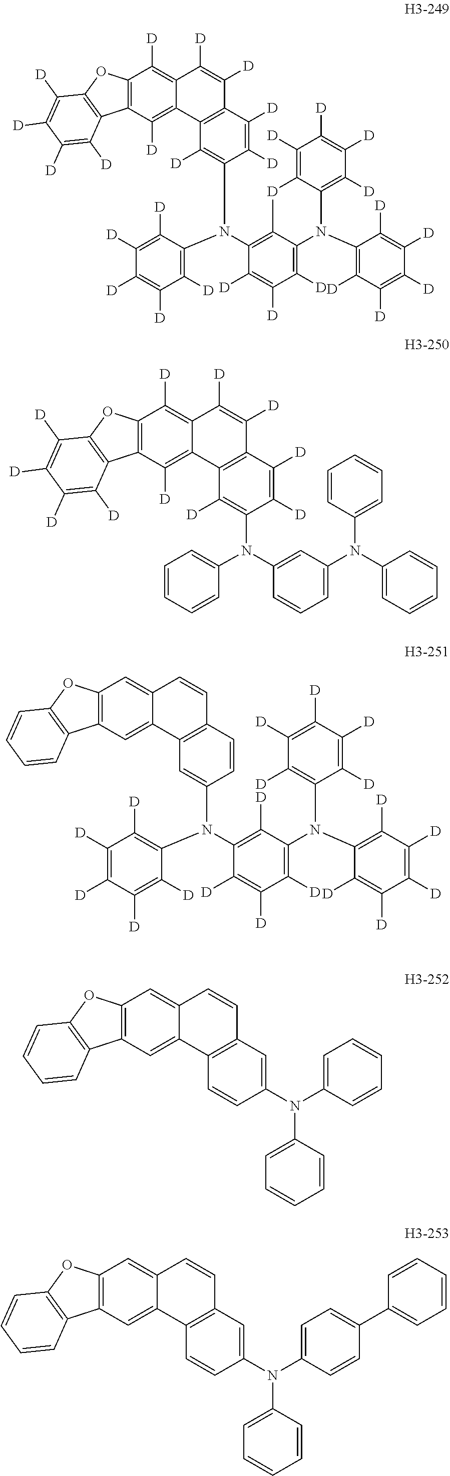 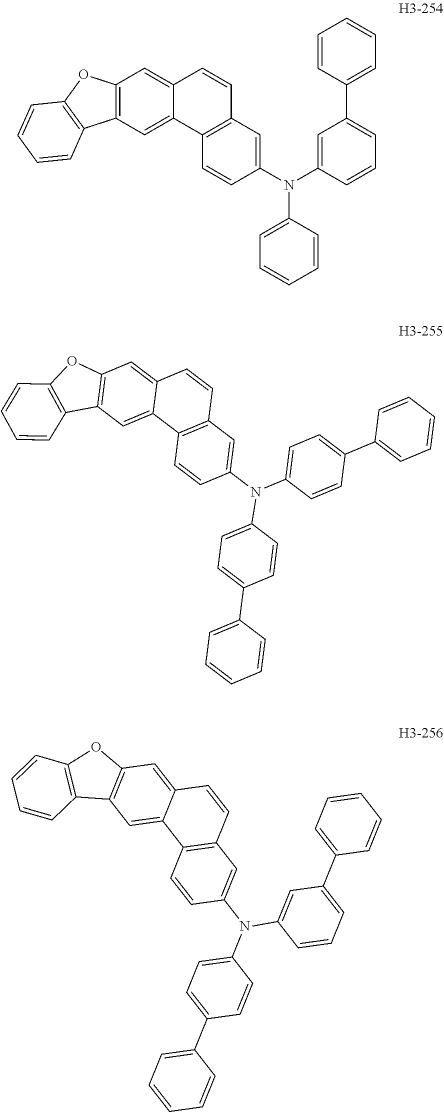 [SMILES: c1ccc(-c2cccc(N(c3ccccc3)c3ccc4c(ccc5cc6oc7ccccc7c6cc54)c3)c2)cc1 ; c1ccc(-c2ccc(N(c3ccc(-c4ccccc4)cc3)c3ccc4c(ccc5cc6oc7ccccc7c6cc54)c3)cc2)cc1 ; c1ccc(-c2ccc(N(c3cccc(-c4ccccc4)c3)c3ccc4c(ccc5cc6oc7ccccc7c6cc54)c3)cc2)cc1] 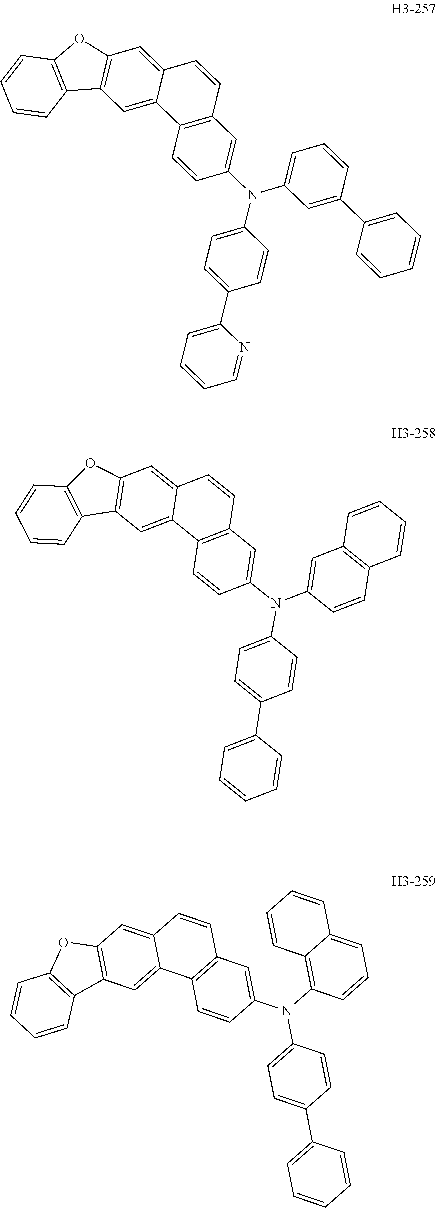 [SMILES: c1ccc(-c2cccc(N(c3ccc(-c4ccccn4)cc3)c3ccc4c(ccc5cc6oc7ccccc7c6cc54)c3)c2)cc1 ; c1ccc(-c2ccc(N(c3ccc4ccccc4c3)c3ccc4c(ccc5cc6oc7ccccc7c6cc54)c3)cc2)cc1 ; c1ccc(-c2ccc(N(c3ccc4c(ccc5cc6oc7ccccc7c6cc54)c3)c3cccc4ccccc34)cc2)cc1] 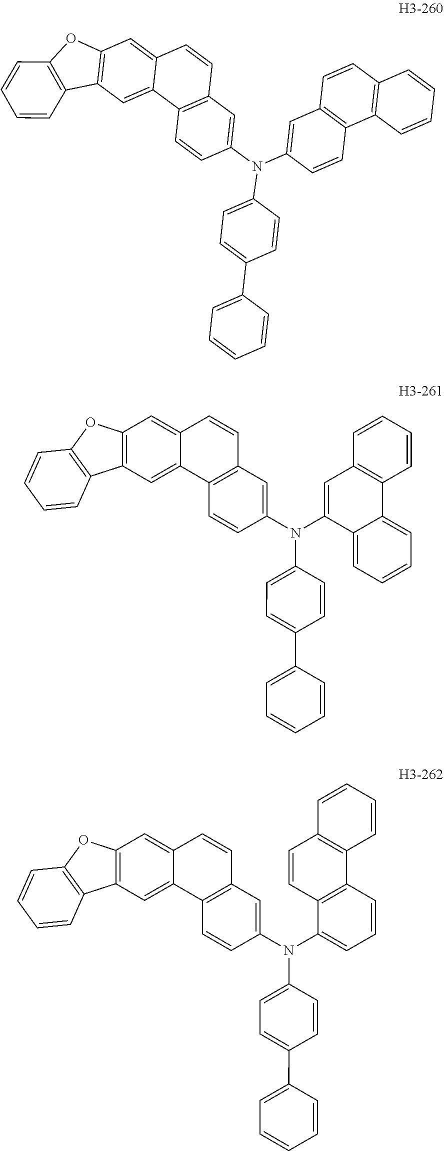 [SMILES: c1ccc(-c2ccc(N(c3ccc4c(ccc5ccccc54)c3)c3ccc4c(ccc5cc6oc7ccccc7c6cc54)c3)cc2)cc1 ; c1ccc(-c2ccc(N(c3ccc4c(ccc5cc6oc7ccccc7c6cc54)c3)c3cc4ccccc4c4ccccc34)cc2)cc1 ; c1ccc(-c2ccc(N(c3ccc4c(ccc5cc6oc7ccccc7c6cc54)c3)c3cccc4c3ccc3ccccc34)cc2)cc1] 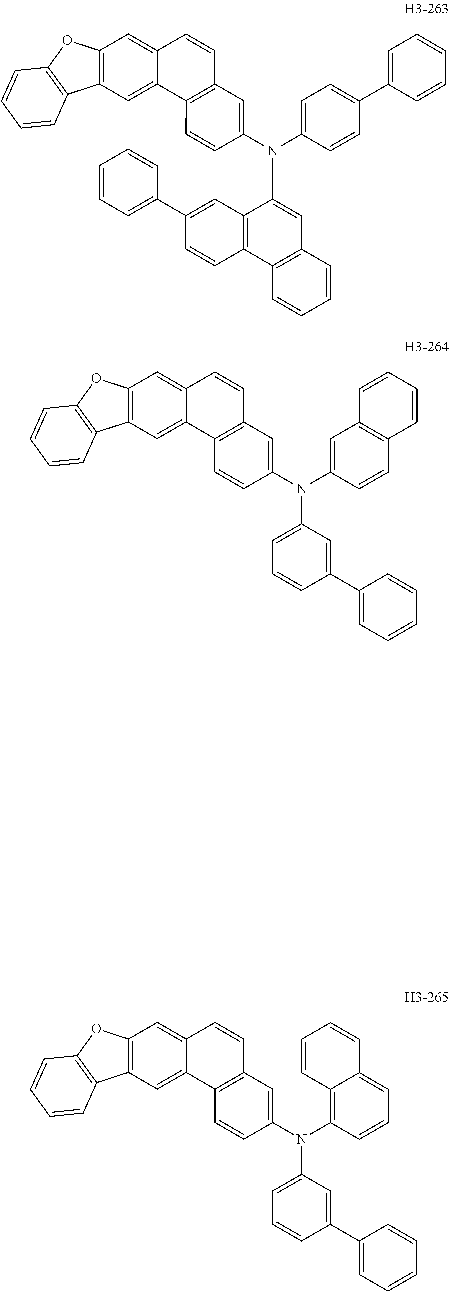 [SMILES: c1ccc(-c2ccc(N(c3ccc4c(ccc5cc6oc7ccccc7c6cc54)c3)c3cc4ccccc4c4ccc(-c5ccccc5)cc34)cc2)cc1 ; c1ccc(-c2cccc(N(c3ccc4ccccc4c3)c3ccc4c(ccc5cc6oc7ccccc7c6cc54)c3)c2)cc1 ; c1ccc(-c2cccc(N(c3ccc4c(ccc5cc6oc7ccccc7c6cc54)c3)c3cccc4ccccc34)c2)cc1] 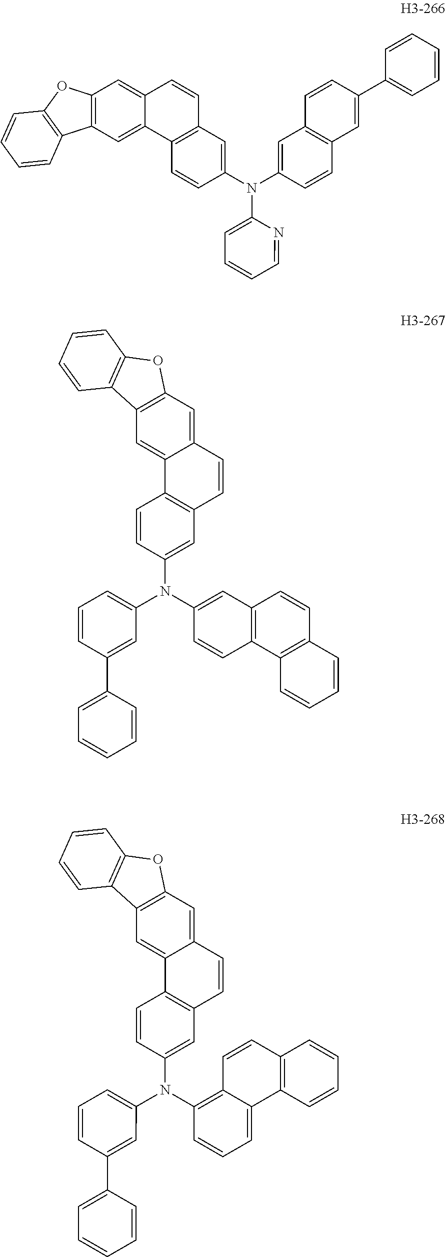 [SMILES: c1ccc(-c2ccc3cc(N(c4ccc5c(ccc6cc7oc8ccccc8c7cc65)c4)c4ccccn4)ccc3c2)cc1 ; c1ccc(-c2cccc(N(c3ccc4c(ccc5ccccc54)c3)c3ccc4c(ccc5cc6oc7ccccc7c6cc54)c3)c2)cc1 ; c1ccc(-c2cccc(N(c3ccc4c(ccc5cc6oc7ccccc7c6cc54)c3)c3cccc4c3ccc3ccccc34)c2)cc1] 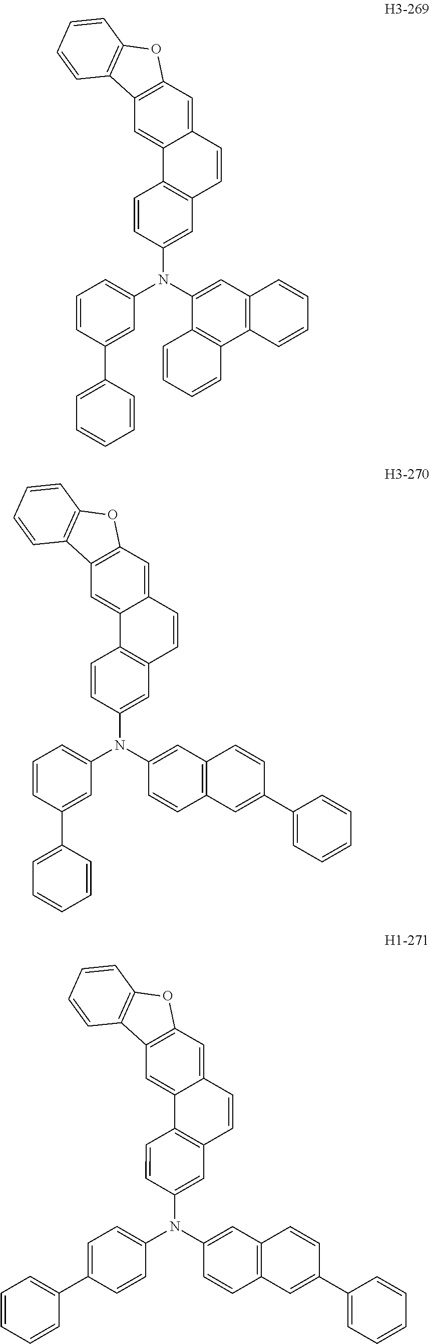 [SMILES: c1ccc(-c2cccc(N(c3ccc4c(ccc5cc6oc7ccccc7c6cc54)c3)c3cc4ccccc4c4ccccc34)c2)cc1 ; c1ccc(-c2cccc(N(c3ccc4cc(-c5ccccc5)ccc4c3)c3ccc4c(ccc5cc6oc7ccccc7c6cc54)c3)c2)cc1 ; c1ccc(-c2ccc(N(c3ccc4cc(-c5ccccc5)ccc4c3)c3ccc4c(ccc5cc6oc7ccccc7c6cc54)c3)cc2)cc1] 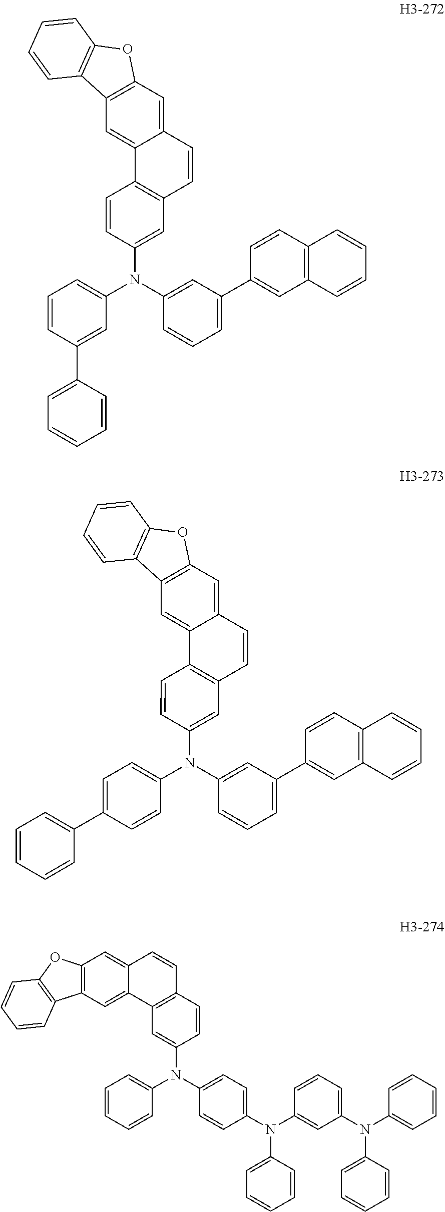 [SMILES: c1ccc(-c2cccc(N(c3cccc(-c4ccc5ccccc5c4)c3)c3ccc4c(ccc5cc6oc7ccccc7c6cc54)c3)c2)cc1 ; c1ccc(-c2ccc(N(c3cccc(-c4ccc5ccccc5c4)c3)c3ccc4c(ccc5cc6oc7ccccc7c6cc54)c3)cc2)cc1 ; c1ccc(N(c2ccccc2)c2cccc(N(c3ccccc3)c3ccc(N(c4ccccc4)c4ccc5ccc6cc7oc8ccccc8c7cc6c5c4)cc3)c2)cc1] 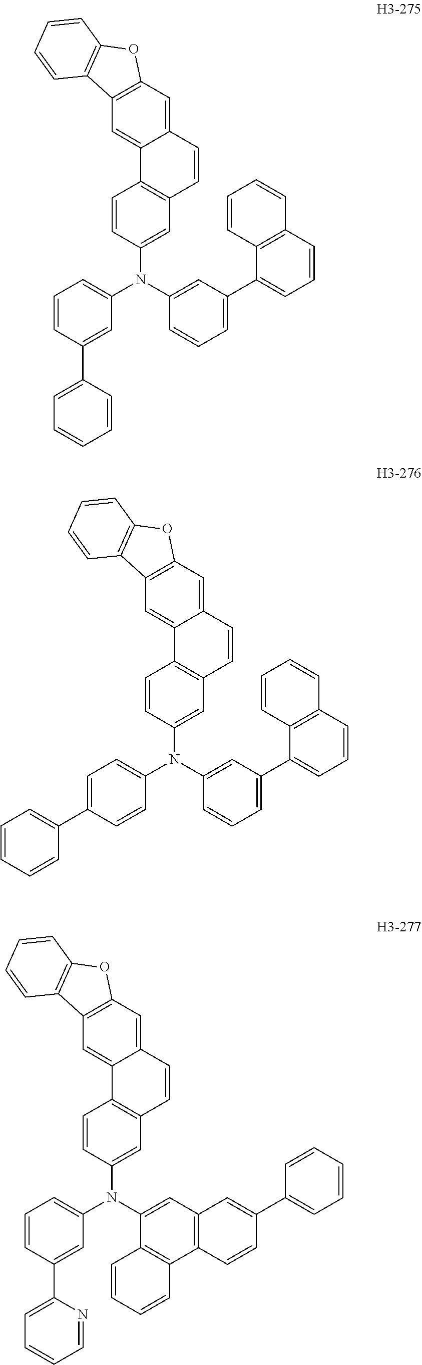 [SMILES: c1ccc(-c2cccc(N(c3cccc(-c4cccc5ccccc45)c3)c3ccc4c(ccc5cc6oc7ccccc7c6cc54)c3)c2)cc1 ; c1ccc(-c2ccc(N(c3cccc(-c4cccc5ccccc45)c3)c3ccc4c(ccc5cc6oc7ccccc7c6cc54)c3)cc2)cc1 ; c1ccc(-c2ccc3c(c2)cc(N(c2cccc(-c4ccccn4)c2)c2ccc4c(ccc5cc6oc7ccccc7c6cc54)c2)c2ccccc23)cc1] 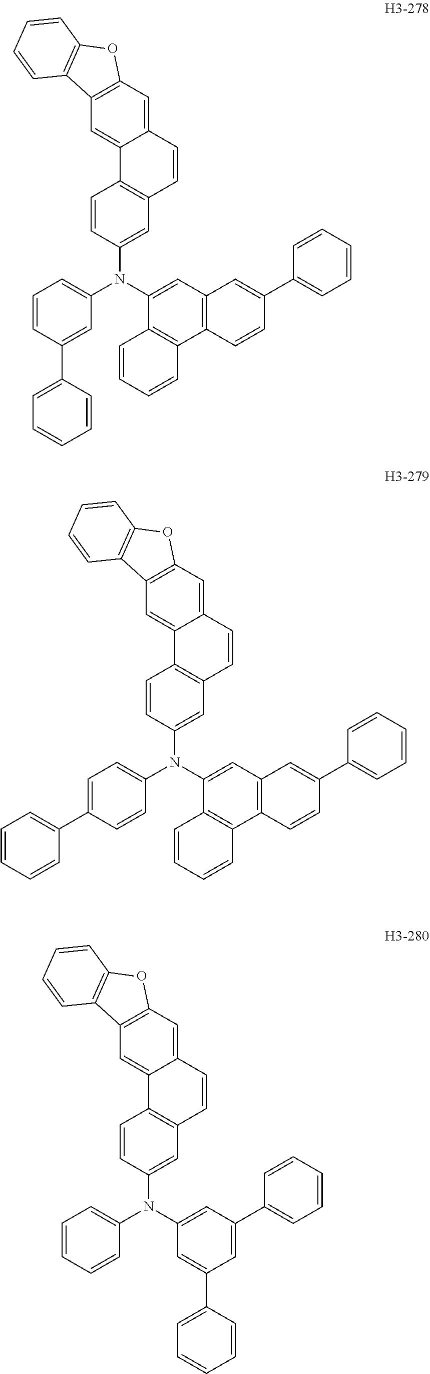 [SMILES: c1ccc(-c2cccc(N(c3ccc4c(ccc5cc6oc7ccccc7c6cc54)c3)c3cc4cc(-c5ccccc5)ccc4c4ccccc34)c2)cc1 ; c1ccc(-c2ccc(N(c3ccc4c(ccc5cc6oc7ccccc7c6cc54)c3)c3cc4cc(-c5ccccc5)ccc4c4ccccc34)cc2)cc1 ; c1ccc(-c2cc(-c3ccccc3)cc(N(c3ccccc3)c3ccc4c(ccc5cc6oc7ccccc7c6cc54)c3)c2)cc1] 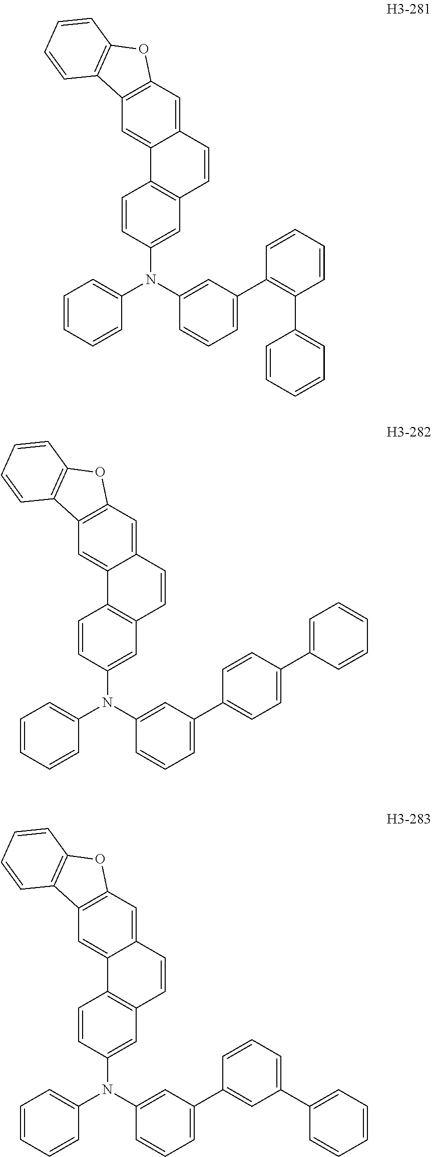 [SMILES: c1ccc(-c2ccccc2-c2cccc(N(c3ccccc3)c3ccc4c(ccc5cc6oc7ccccc7c6cc54)c3)c2)cc1 ; c1ccc(-c2ccc(-c3cccc(N(c4ccccc4)c4ccc5c(ccc6cc7oc8ccccc8c7cc65)c4)c3)cc2)cc1 ; c1ccc(-c2cccc(-c3cccc(N(c4ccccc4)c4ccc5c(ccc6cc7oc8ccccc8c7cc65)c4)c3)c2)cc1] 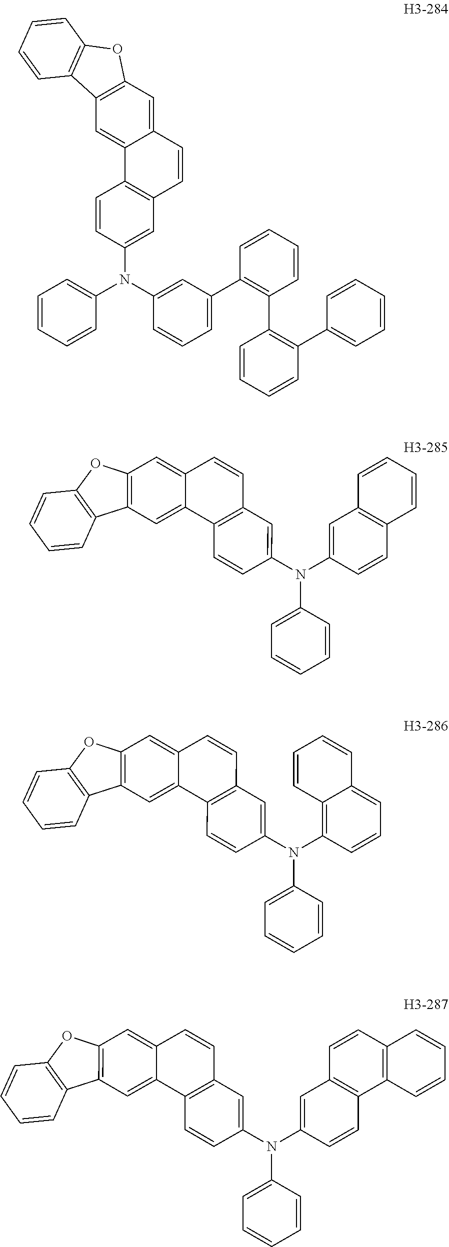 [SMILES: c1ccc(-c2ccccc2-c2ccccc2-c2cccc(N(c3ccccc3)c3ccc4c(ccc5cc6oc7ccccc7c6cc54)c3)c2)cc1 ; c1ccc(N(c2ccc3ccccc3c2)c2ccc3c(ccc4cc5oc6ccccc6c5cc43)c2)cc1 ; c1ccc(N(c2ccc3c(ccc4cc5oc6ccccc6c5cc43)c2)c2cccc3ccccc23)cc1 ; c1ccc(N(c2ccc3c(ccc4ccccc43)c2)c2ccc3c(ccc4cc5oc6ccccc6c5cc43)c2)cc1] 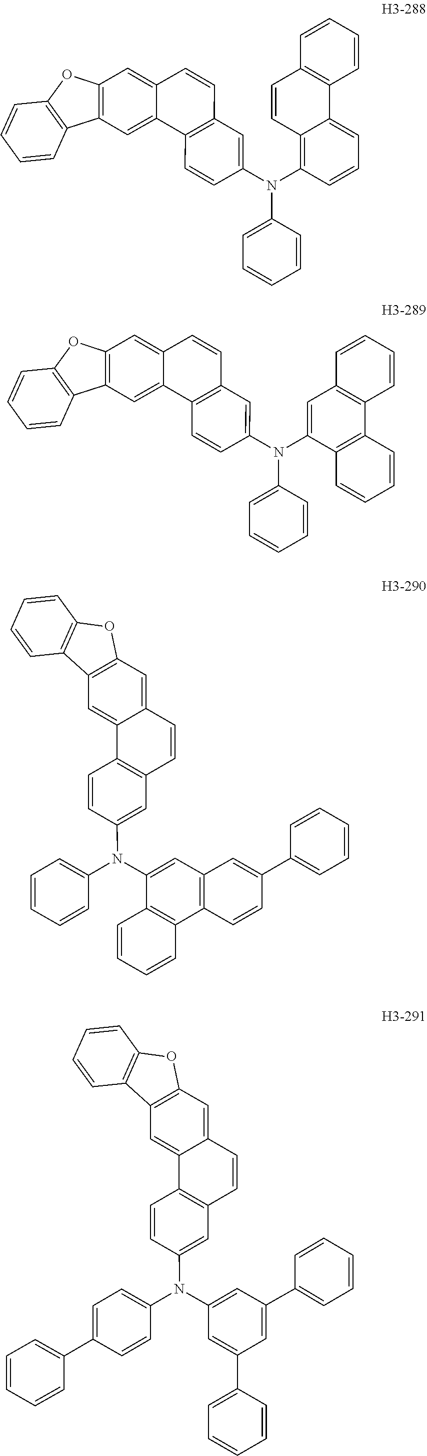 [SMILES: c1ccc(N(c2ccc3c(ccc4cc5oc6ccccc6c5cc43)c2)c2cccc3c2ccc2ccccc23)cc1 ; c1ccc(N(c2ccc3c(ccc4cc5oc6ccccc6c5cc43)c2)c2cc3ccccc3c3ccccc23)cc1 ; c1ccc(-c2ccc3c(c2)cc(N(c2ccccc2)c2ccc4c(ccc5cc6oc7ccccc7c6cc54)c2)c2ccccc23)cc1 ; c1ccc(-c2ccc(N(c3cc(-c4ccccc4)cc(-c4ccccc4)c3)c3ccc4c(ccc5cc6oc7ccccc7c6cc54)c3)cc2)cc1] 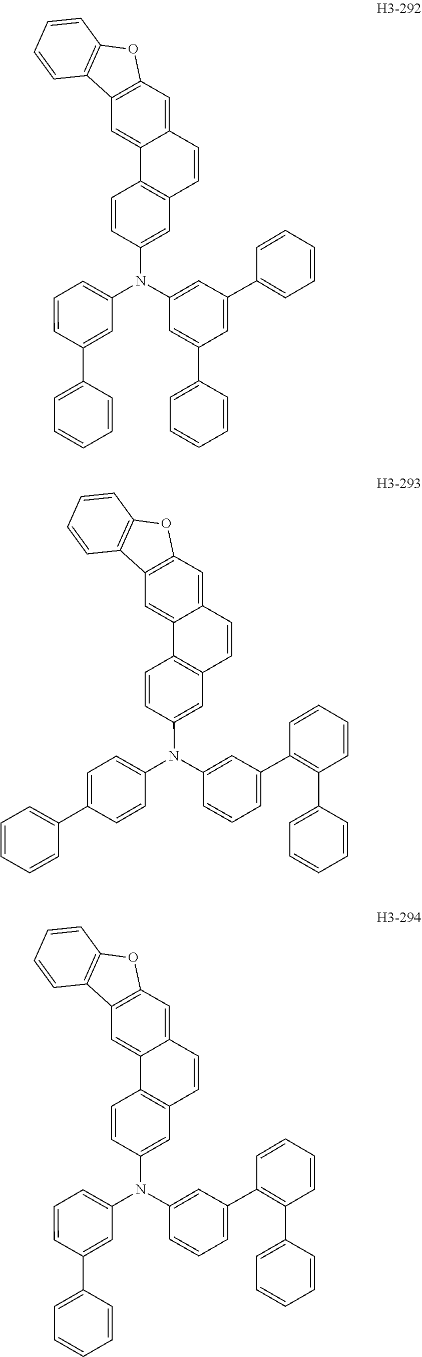 [SMILES: c1ccc(-c2cccc(N(c3cc(-c4ccccc4)cc(-c4ccccc4)c3)c3ccc4c(ccc5cc6oc7ccccc7c6cc54)c3)c2)cc1 ; c1ccc(-c2ccc(N(c3cccc(-c4ccccc4-c4ccccc4)c3)c3ccc4c(ccc5cc6oc7ccccc7c6cc54)c3)cc2)cc1 ; c1ccc(-c2cccc(N(c3cccc(-c4ccccc4-c4ccccc4)c3)c3ccc4c(ccc5cc6oc7ccccc7c6cc54)c3)c2)cc1] 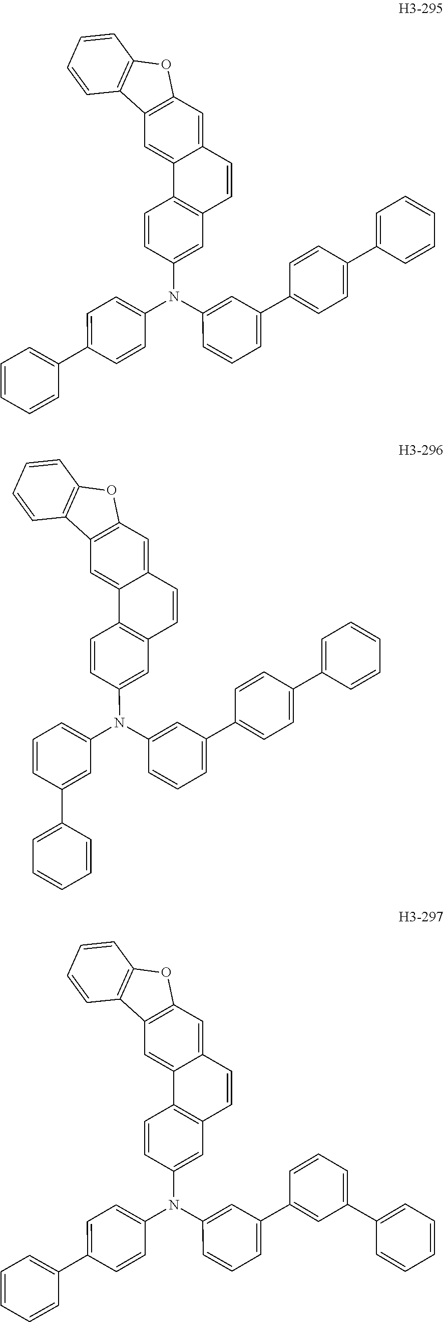 [SMILES: c1ccc(-c2ccc(-c3cccc(N(c4ccc(-c5ccccc5)cc4)c4ccc5c(ccc6cc7oc8ccccc8c7cc65)c4)c3)cc2)cc1 ; c1ccc(-c2ccc(-c3cccc(N(c4cccc(-c5ccccc5)c4)c4ccc5c(ccc6cc7oc8ccccc8c7cc65)c4)c3)cc2)cc1 ; c1ccc(-c2ccc(N(c3cccc(-c4cccc(-c5ccccc5)c4)c3)c3ccc4c(ccc5cc6oc7ccccc7c6cc54)c3)cc2)cc1] 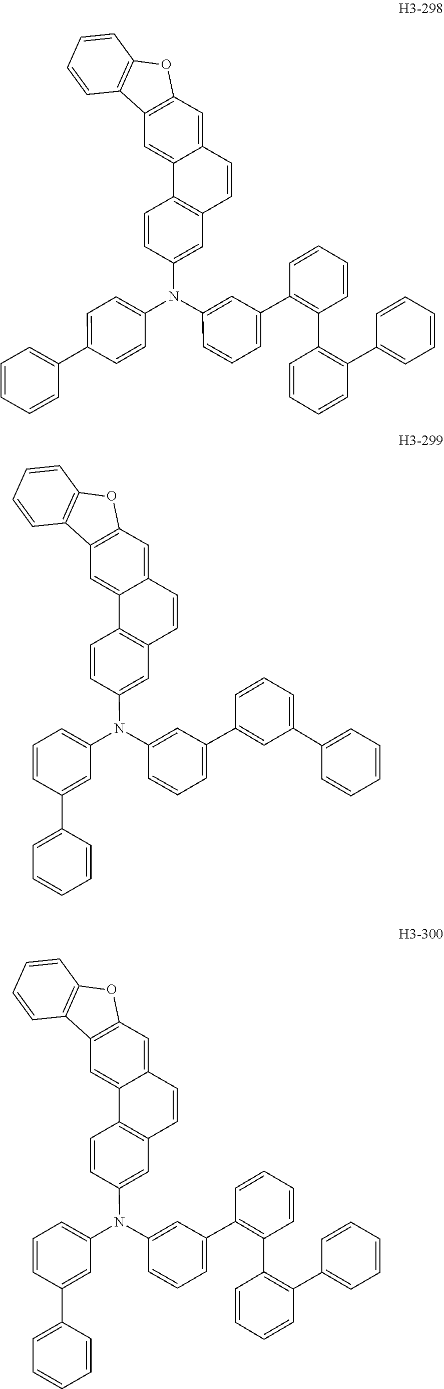 [SMILES: c1ccc(-c2ccc(N(c3cccc(-c4ccccc4-c4ccccc4-c4ccccc4)c3)c3ccc4c(ccc5cc6oc7ccccc7c6cc54)c3)cc2)cc1 ; c1ccc(-c2cccc(-c3cccc(N(c4cccc(-c5ccccc5)c4)c4ccc5c(ccc6cc7oc8ccccc8c7cc65)c4)c3)c2)cc1 ; c1ccc(-c2cccc(N(c3cccc(-c4ccccc4-c4ccccc4-c4ccccc4)c3)c3ccc4c(ccc5cc6oc7ccccc7c6cc54)c3)c2)cc1] 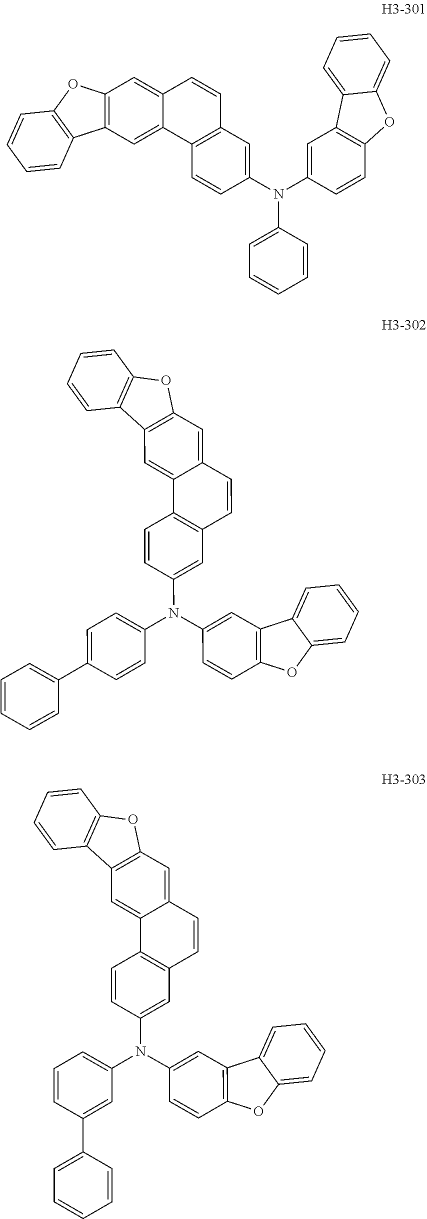 [SMILES: c1ccc(N(c2ccc3c(ccc4cc5oc6ccccc6c5cc43)c2)c2ccc3oc4ccccc4c3c2)cc1 ; c1ccc(-c2ccc(N(c3ccc4c(ccc5cc6oc7ccccc7c6cc54)c3)c3ccc4oc5ccccc5c4c3)cc2)cc1 ; c1ccc(-c2cccc(N(c3ccc4c(ccc5cc6oc7ccccc7c6cc54)c3)c3ccc4oc5ccccc5c4c3)c2)cc1] 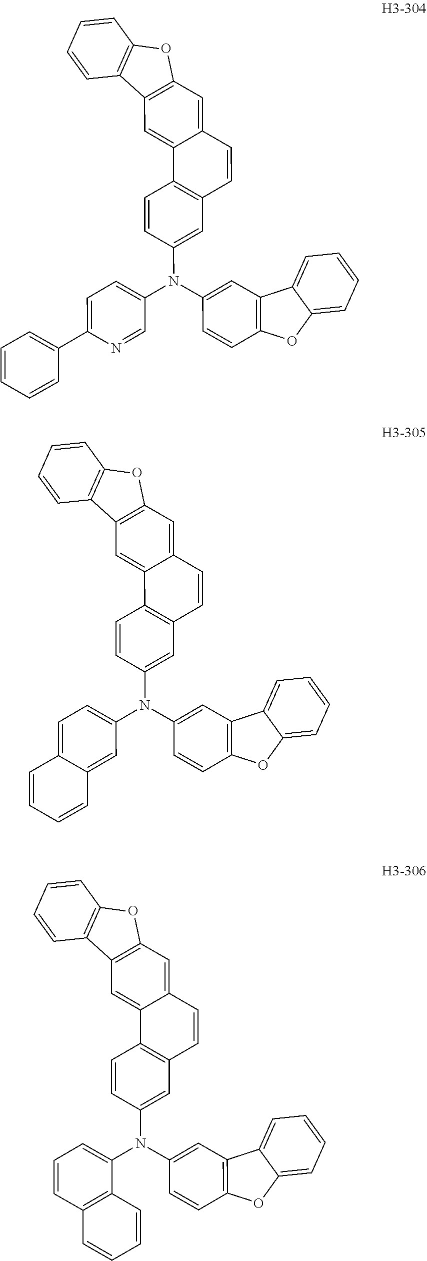 [SMILES: c1ccc(-c2ccc(N(c3ccc4c(ccc5cc6oc7ccccc7c6cc54)c3)c3ccc4oc5ccccc5c4c3)cn2)cc1 ; c1ccc2cc(N(c3ccc4c(ccc5cc6oc7ccccc7c6cc54)c3)c3ccc4oc5ccccc5c4c3)ccc2c1 ; c1ccc2c(N(c3ccc4c(ccc5cc6oc7ccccc7c6cc54)c3)c3ccc4oc5ccccc5c4c3)cccc2c1] 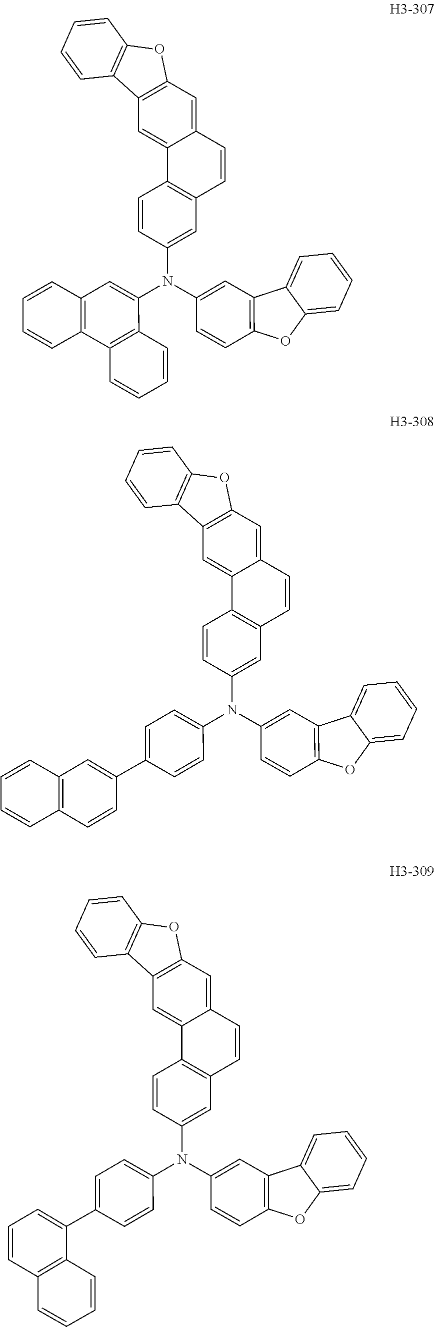 [SMILES: c1ccc2c(c1)cc(N(c1ccc3c(ccc4cc5oc6ccccc6c5cc43)c1)c1ccc3oc4ccccc4c3c1)c1ccccc12 ; c1ccc2cc(-c3ccc(N(c4ccc5c(ccc6cc7oc8ccccc8c7cc65)c4)c4ccc5oc6ccccc6c5c4)cc3)ccc2c1 ; c1ccc2c(-c3ccc(N(c4ccc5c(ccc6cc7oc8ccccc8c7cc65)c4)c4ccc5oc6ccccc6c5c4)cc3)cccc2c1] 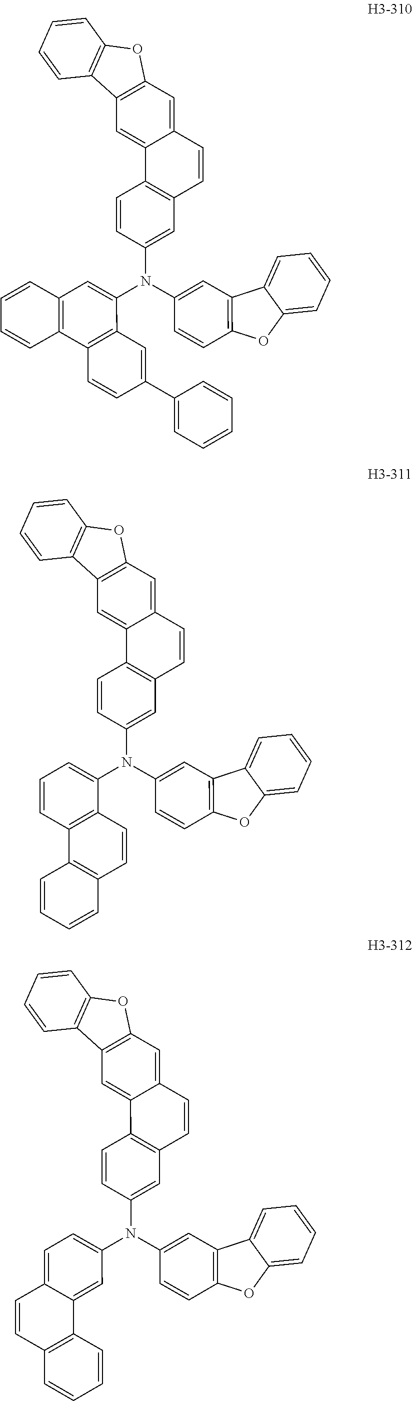 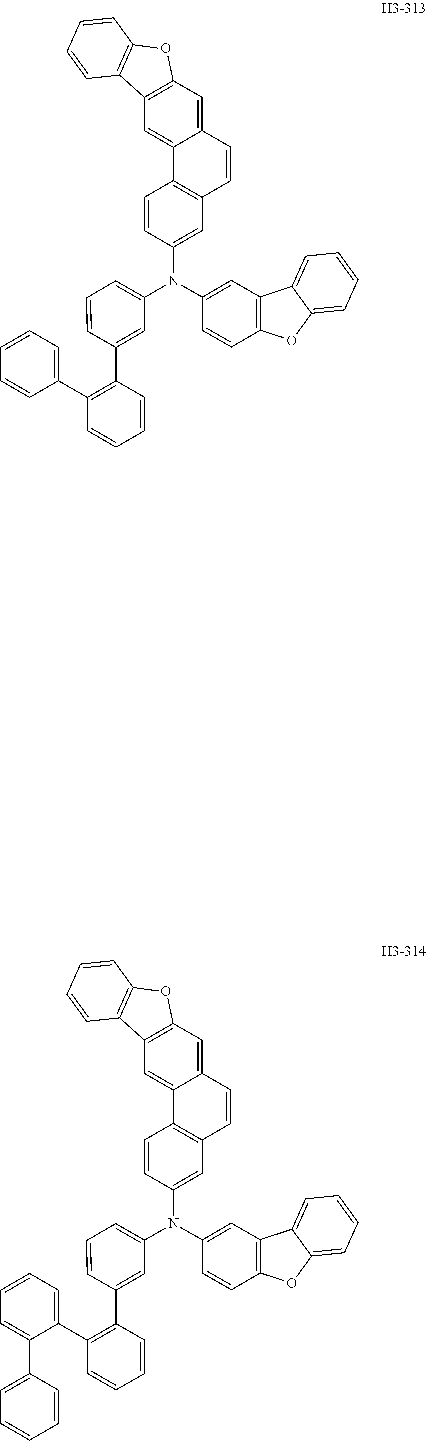 [SMILES: c1ccc(-c2ccccc2-c2cccc(N(c3ccc4c(ccc5cc6oc7ccccc7c6cc54)c3)c3ccc4oc5ccccc5c4c3)c2)cc1 ; c1ccc(-c2ccccc2-c2ccccc2-c2cccc(N(c3ccc4c(ccc5cc6oc7ccccc7c6cc54)c3)c3ccc4oc5ccccc5c4c3)c2)cc1] 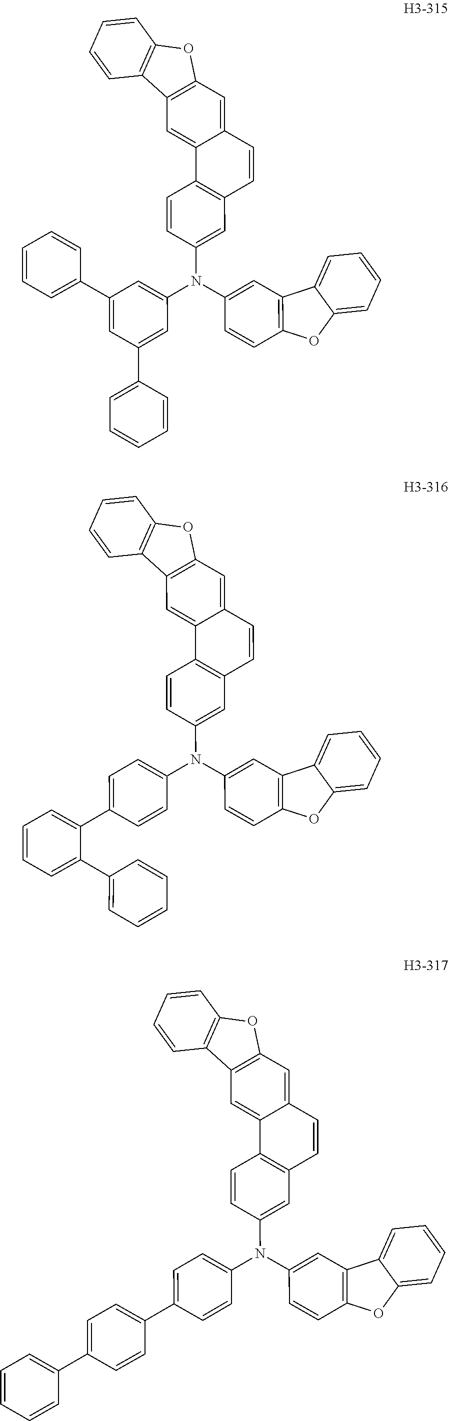 [SMILES: c1ccc(-c2cc(-c3ccccc3)cc(N(c3ccc4c(ccc5cc6oc7ccccc7c6cc54)c3)c3ccc4oc5ccccc5c4c3)c2)cc1 ; c1ccc(-c2ccccc2-c2ccc(N(c3ccc4c(ccc5cc6oc7ccccc7c6cc54)c3)c3ccc4oc5ccccc5c4c3)cc2)cc1 ; c1ccc(-c2ccc(-c3ccc(N(c4ccc5c(ccc6cc7oc8ccccc8c7cc65)c4)c4ccc5oc6ccccc6c5c4)cc3)cc2)cc1] 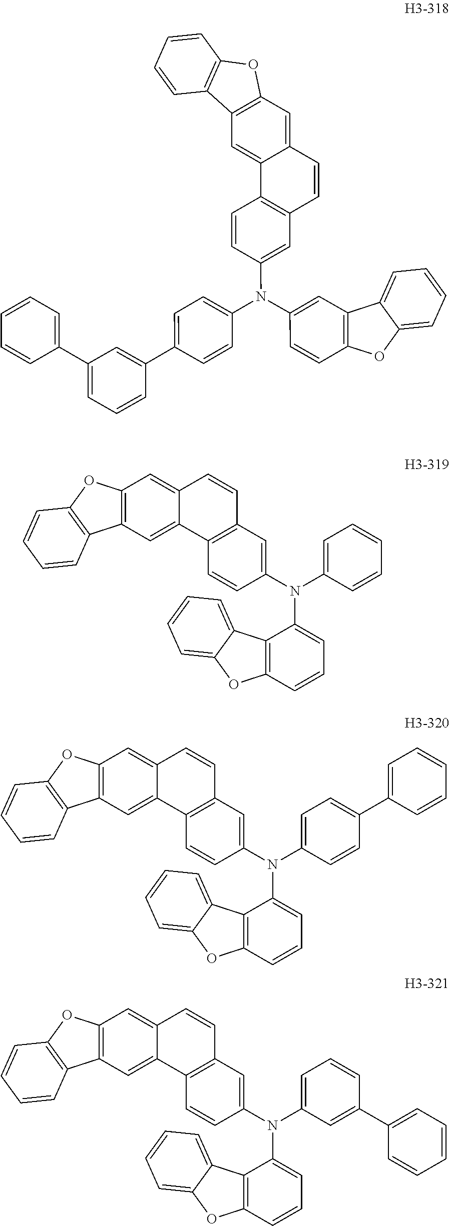 [SMILES: c1ccc(-c2cccc(-c3ccc(N(c4ccc5c(ccc6cc7oc8ccccc8c7cc65)c4)c4ccc5oc6ccccc6c5c4)cc3)c2)cc1 ; c1ccc(N(c2ccc3c(ccc4cc5oc6ccccc6c5cc43)c2)c2cccc3oc4ccccc4c23)cc1 ; c1ccc(-c2ccc(N(c3ccc4c(ccc5cc6oc7ccccc7c6cc54)c3)c3cccc4oc5ccccc5c34)cc2)cc1 ; c1ccc(-c2cccc(N(c3ccc4c(ccc5cc6oc7ccccc7c6cc54)c3)c3cccc4oc5ccccc5c34)c2)cc1] 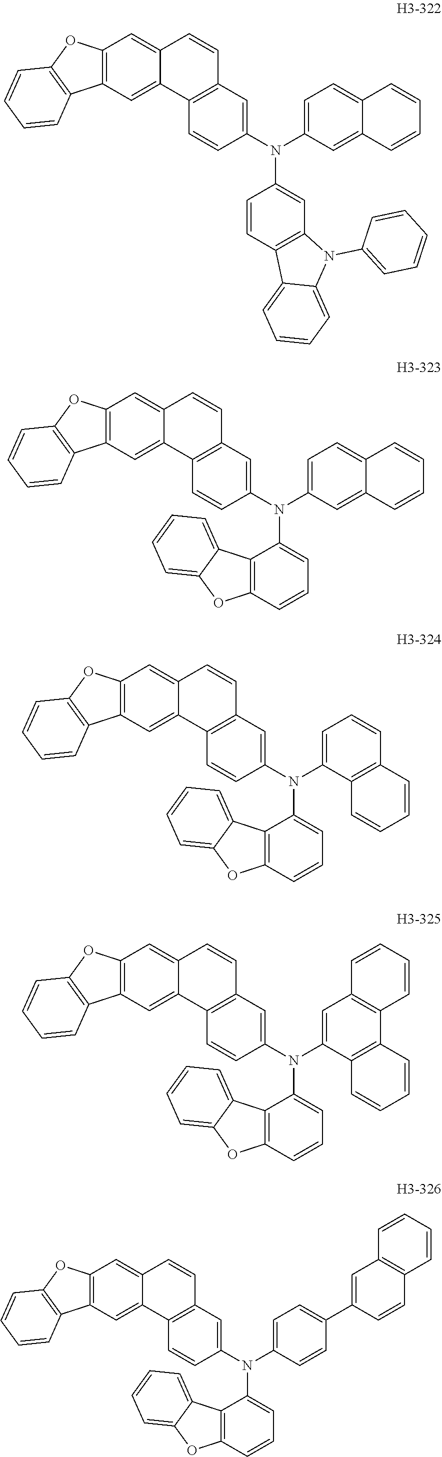 [SMILES: c1ccc(-n2c3ccccc3c3ccc(N(c4ccc5ccccc5c4)c4ccc5c(ccc6cc7oc8ccccc8c7cc65)c4)cc32)cc1 ; c1ccc2cc(N(c3ccc4c(ccc5cc6oc7ccccc7c6cc54)c3)c3cccc4oc5ccccc5c34)ccc2c1 ; c1ccc2c(N(c3ccc4c(ccc5cc6oc7ccccc7c6cc54)c3)c3cccc4oc5ccccc5c34)cccc2c1 ; c1ccc2c(c1)cc(N(c1ccc3c(ccc4cc5oc6ccccc6c5cc43)c1)c1cccc3oc4ccccc4c13)c1ccccc12 ; c1ccc2cc(-c3ccc(N(c4ccc5c(ccc6cc7oc8ccccc8c7cc65)c4)c4cccc5oc6ccccc6c45)cc3)ccc2c1] 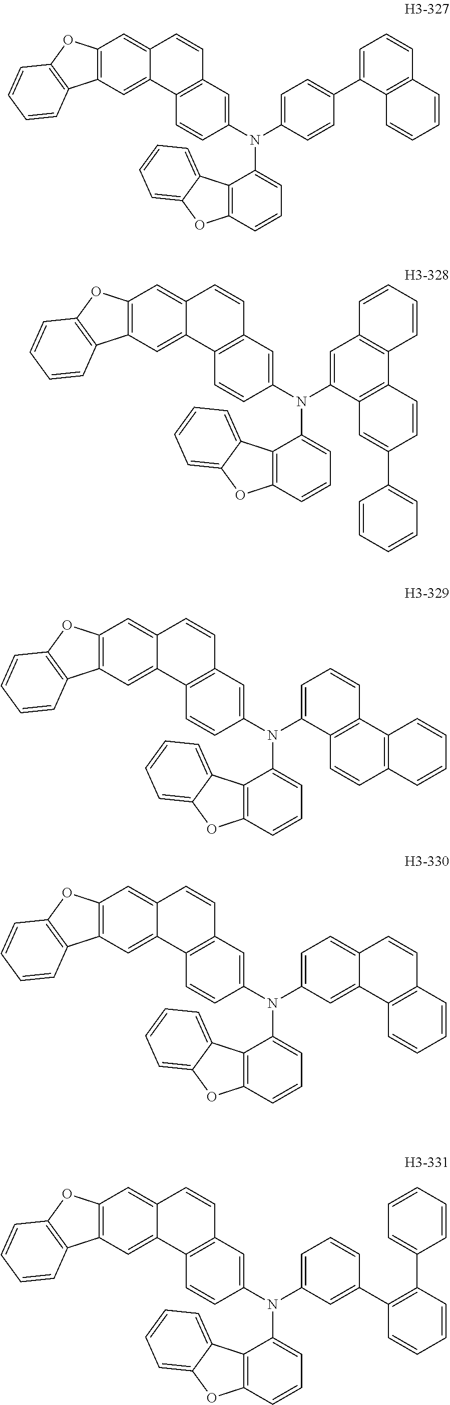 [SMILES: c1ccc2c(-c3ccc(N(c4ccc5c(ccc6cc7oc8ccccc8c7cc65)c4)c4cccc5oc6ccccc6c45)cc3)cccc2c1 ; c1ccc(-c2ccc3c(c2)c(N(c2ccc4c(ccc5cc6oc7ccccc7c6cc54)c2)c2cccc4oc5ccccc5c24)cc2ccccc23)cc1 ; c1ccc2c(c1)ccc1c(N(c3ccc4c(ccc5cc6oc7ccccc7c6cc54)c3)c3cccc4oc5ccccc5c34)cccc12 ; c1ccc2c(c1)ccc1ccc(N(c3ccc4c(ccc5cc6oc7ccccc7c6cc54)c3)c3cccc4oc5ccccc5c34)cc12 ; c1ccc(-c2ccccc2-c2cccc(N(c3ccc4c(ccc5cc6oc7ccccc7c6cc54)c3)c3cccc4oc5ccccc5c34)c2)cc1] 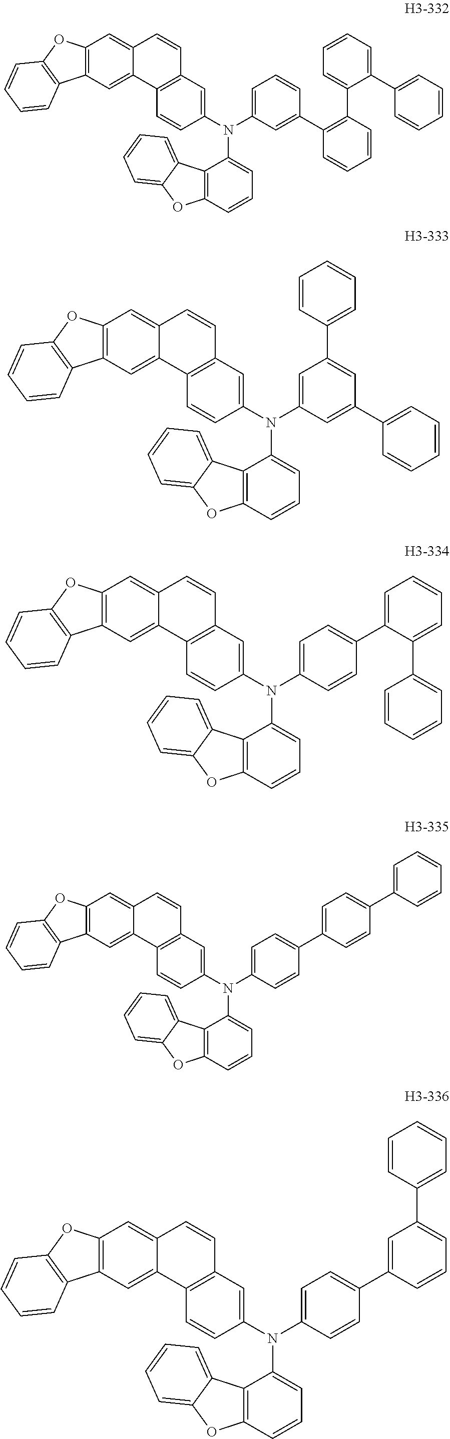 [SMILES: c1ccc(-c2ccccc2-c2ccccc2-c2cccc(N(c3ccc4c(ccc5cc6oc7ccccc7c6cc54)c3)c3cccc4oc5ccccc5c34)c2)cc1 ; c1ccc(-c2cc(-c3ccccc3)cc(N(c3ccc4c(ccc5cc6oc7ccccc7c6cc54)c3)c3cccc4oc5ccccc5c34)c2)cc1 ; c1ccc(-c2ccccc2-c2ccc(N(c3ccc4c(ccc5cc6oc7ccccc7c6cc54)c3)c3cccc4oc5ccccc5c34)cc2)cc1 ; c1ccc(-c2ccc(-c3ccc(N(c4ccc5c(ccc6cc7oc8ccccc8c7cc65)c4)c4cccc5oc6ccccc6c45)cc3)cc2)cc1 ; c1ccc(-c2cccc(-c3ccc(N(c4ccc5c(ccc6cc7oc8ccccc8c7cc65)c4)c4cccc5oc6ccccc6c45)cc3)c2)cc1] 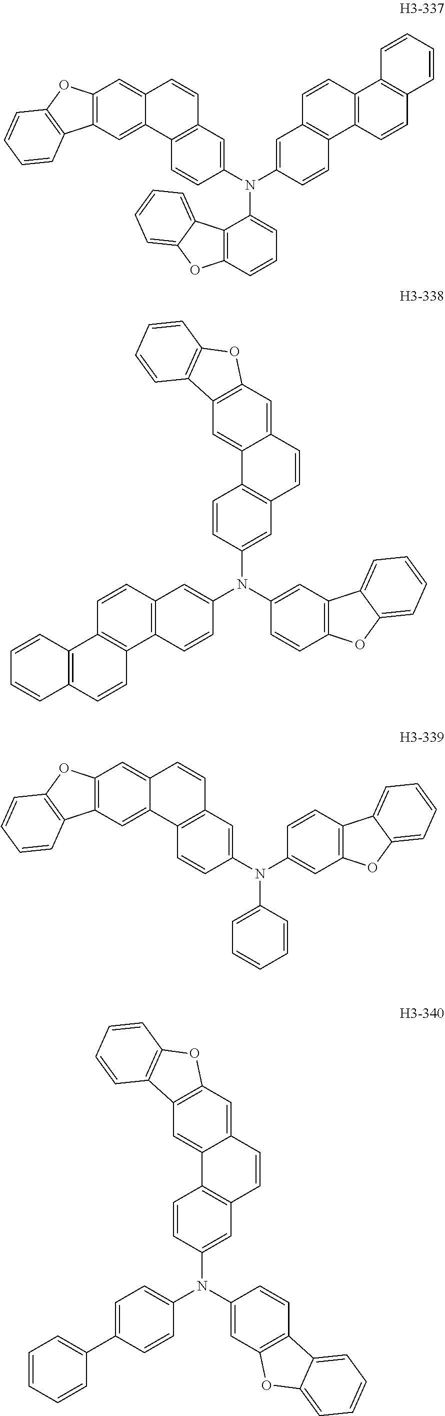 [SMILES: c1ccc2c(c1)ccc1c3ccc(N(c4ccc5c(ccc6cc7oc8ccccc8c7cc65)c4)c4cccc5oc6ccccc6c45)cc3ccc21 ; c1ccc2c(c1)ccc1c3ccc(N(c4ccc5c(ccc6cc7oc8ccccc8c7cc65)c4)c4ccc5oc6ccccc6c5c4)cc3ccc21 ; c1ccc(N(c2ccc3c(ccc4cc5oc6ccccc6c5cc43)c2)c2ccc3c(c2)oc2ccccc23)cc1 ; c1ccc(-c2ccc(N(c3ccc4c(ccc5cc6oc7ccccc7c6cc54)c3)c3ccc4c(c3)oc3ccccc34)cc2)cc1] 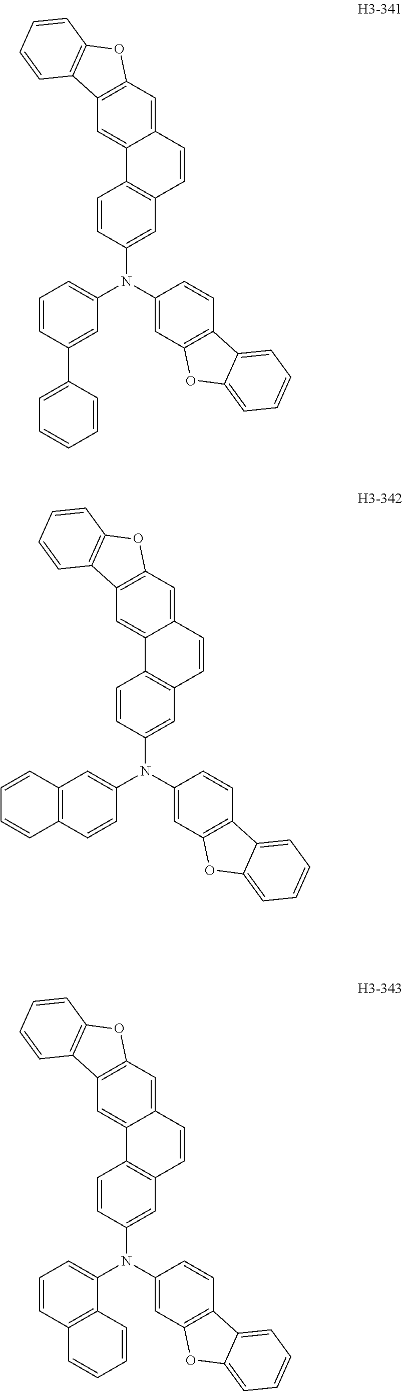 [SMILES: c1ccc(-c2cccc(N(c3ccc4c(ccc5cc6oc7ccccc7c6cc54)c3)c3ccc4c(c3)oc3ccccc34)c2)cc1 ; c1ccc2cc(N(c3ccc4c(ccc5cc6oc7ccccc7c6cc54)c3)c3ccc4c(c3)oc3ccccc34)ccc2c1 ; c1ccc2c(N(c3ccc4c(ccc5cc6oc7ccccc7c6cc54)c3)c3ccc4c(c3)oc3ccccc34)cccc2c1] 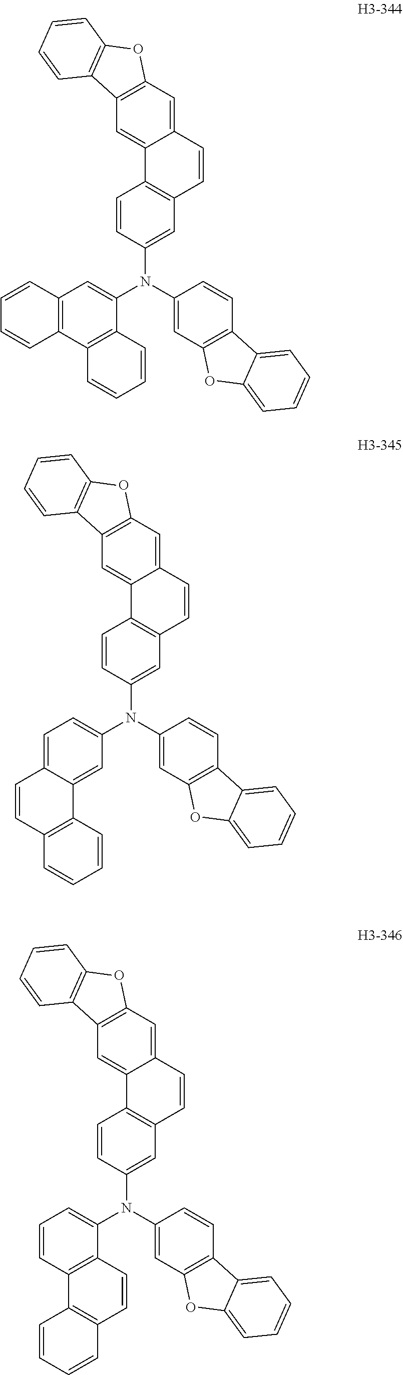 [SMILES: c1ccc2c(c1)cc(N(c1ccc3c(ccc4cc5oc6ccccc6c5cc43)c1)c1ccc3c(c1)oc1ccccc13)c1ccccc12 ; c1ccc2c(c1)ccc1ccc(N(c3ccc4c(ccc5cc6oc7ccccc7c6cc54)c3)c3ccc4c(c3)oc3ccccc34)cc12 ; c1ccc2c(c1)ccc1c(N(c3ccc4c(ccc5cc6oc7ccccc7c6cc54)c3)c3ccc4c(c3)oc3ccccc34)cccc12] 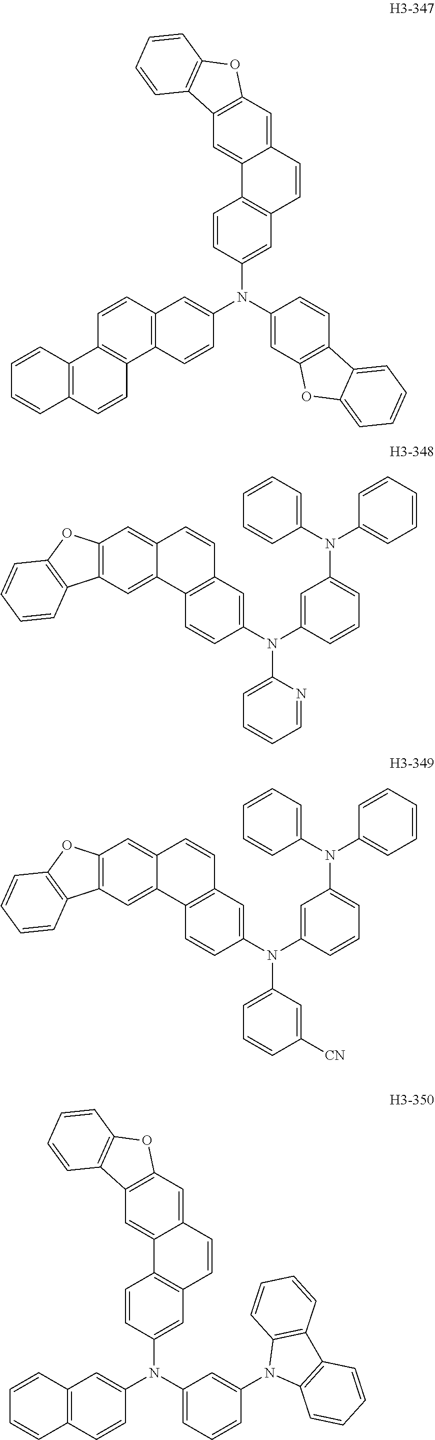 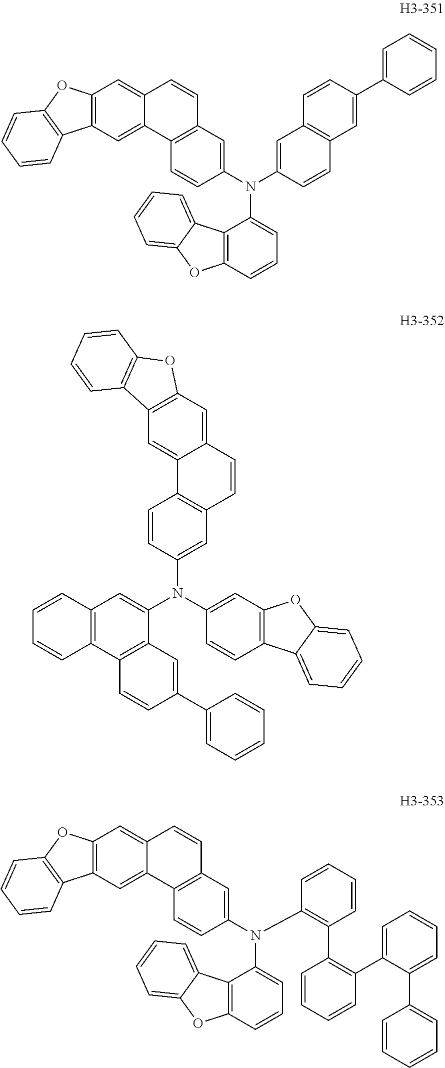 [SMILES: c1ccc(-c2ccc3cc(N(c4ccc5c(ccc6cc7oc8ccccc8c7cc65)c4)c4cccc5oc6ccccc6c45)ccc3c2)cc1 ; c1ccc(-c2ccc3c(c2)c(N(c2ccc4c(ccc5cc6oc7ccccc7c6cc54)c2)c2ccc4c(c2)oc2ccccc24)cc2ccccc23)cc1 ; c1ccc(-c2ccccc2-c2ccccc2-c2ccccc2N(c2ccc3c(ccc4cc5oc6ccccc6c5cc43)c2)c2cccc3oc4ccccc4c23)cc1] 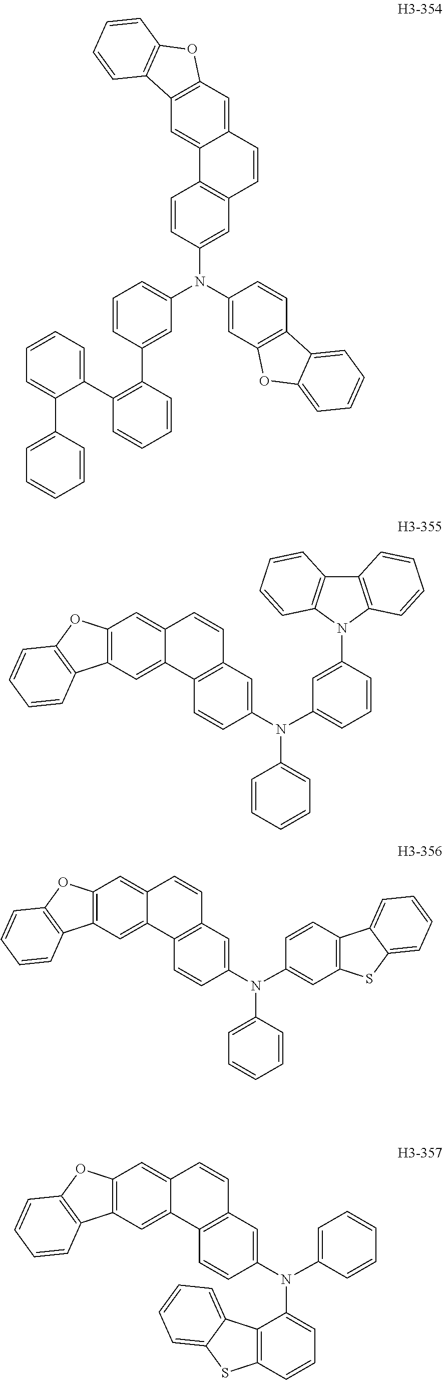 [SMILES: c1ccc(-c2ccccc2-c2ccccc2-c2cccc(N(c3ccc4c(ccc5cc6oc7ccccc7c6cc54)c3)c3ccc4c(c3)oc3ccccc34)c2)cc1 ; c1ccc(N(c2cccc(-n3c4ccccc4c4ccccc43)c2)c2ccc3c(ccc4cc5oc6ccccc6c5cc43)c2)cc1 ; c1ccc(N(c2ccc3c(ccc4cc5oc6ccccc6c5cc43)c2)c2ccc3c(c2)sc2ccccc23)cc1 ; c1ccc(N(c2ccc3c(ccc4cc5oc6ccccc6c5cc43)c2)c2cccc3sc4ccccc4c23)cc1] 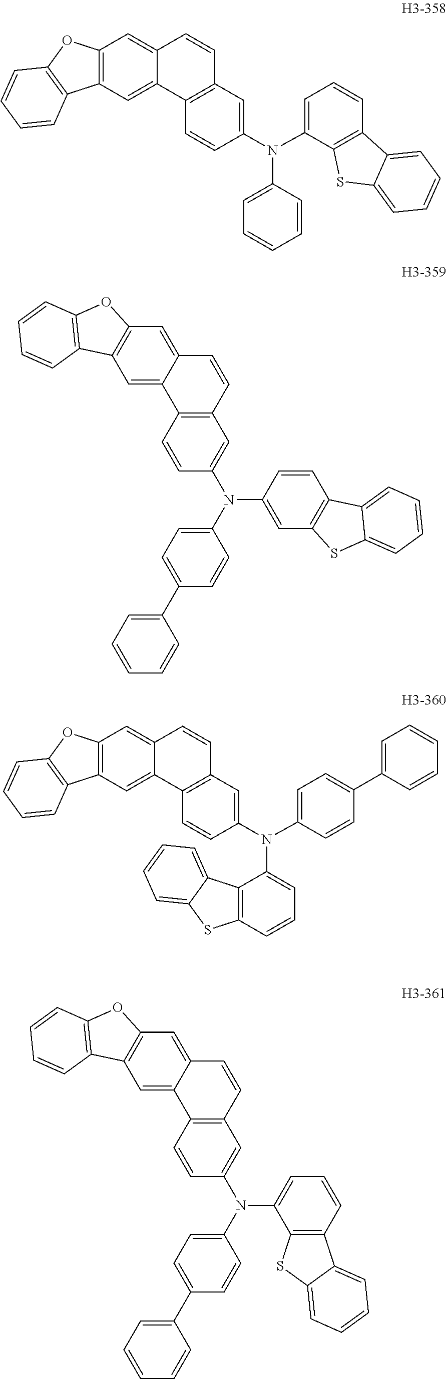 [SMILES: c1ccc(N(c2ccc3c(ccc4cc5oc6ccccc6c5cc43)c2)c2cccc3c2sc2ccccc23)cc1 ; c1ccc(-c2ccc(N(c3ccc4c(ccc5cc6oc7ccccc7c6cc54)c3)c3ccc4c(c3)sc3ccccc34)cc2)cc1 ; c1ccc(-c2ccc(N(c3ccc4c(ccc5cc6oc7ccccc7c6cc54)c3)c3cccc4sc5ccccc5c34)cc2)cc1 ; c1ccc(-c2ccc(N(c3ccc4c(ccc5cc6oc7ccccc7c6cc54)c3)c3cccc4c3sc3ccccc34)cc2)cc1] 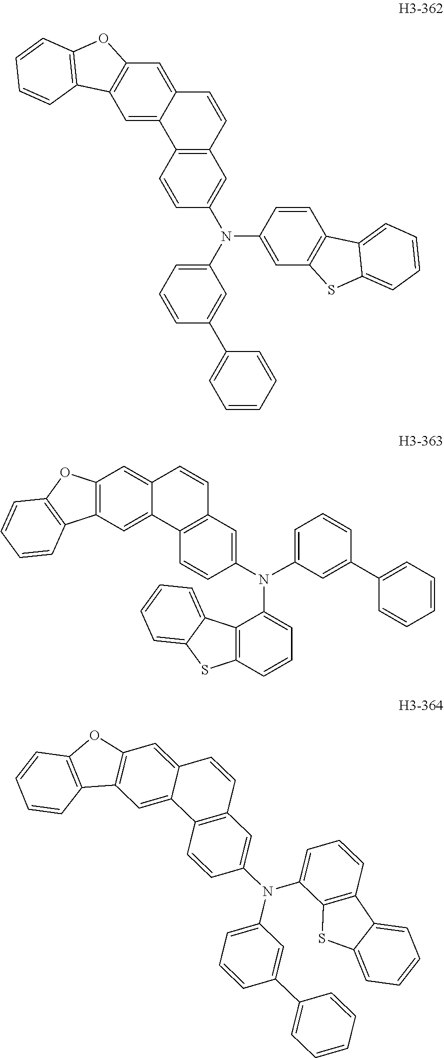 [SMILES: c1ccc(-c2cccc(N(c3ccc4c(ccc5cc6oc7ccccc7c6cc54)c3)c3ccc4c(c3)sc3ccccc34)c2)cc1 ; c1ccc(-c2cccc(N(c3ccc4c(ccc5cc6oc7ccccc7c6cc54)c3)c3cccc4sc5ccccc5c34)c2)cc1 ; c1ccc(-c2cccc(N(c3ccc4c(ccc5cc6oc7ccccc7c6cc54)c3)c3cccc4c3sc3ccccc34)c2)cc1] 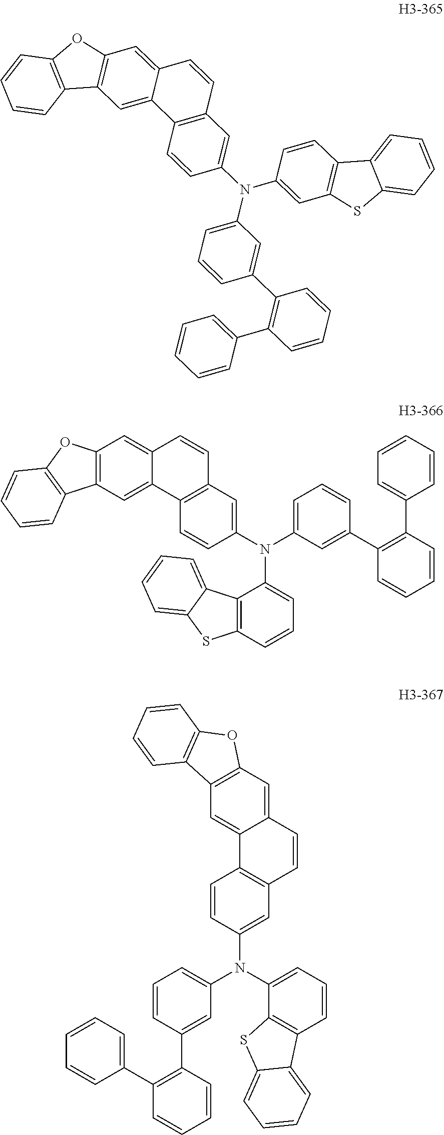 [SMILES: c1ccc(-c2ccccc2-c2cccc(N(c3ccc4c(ccc5cc6oc7ccccc7c6cc54)c3)c3ccc4c(c3)sc3ccccc34)c2)cc1 ; c1ccc(-c2ccccc2-c2cccc(N(c3ccc4c(ccc5cc6oc7ccccc7c6cc54)c3)c3cccc4sc5ccccc5c34)c2)cc1 ; c1ccc(-c2ccccc2-c2cccc(N(c3ccc4c(ccc5cc6oc7ccccc7c6cc54)c3)c3cccc4c3sc3ccccc34)c2)cc1] 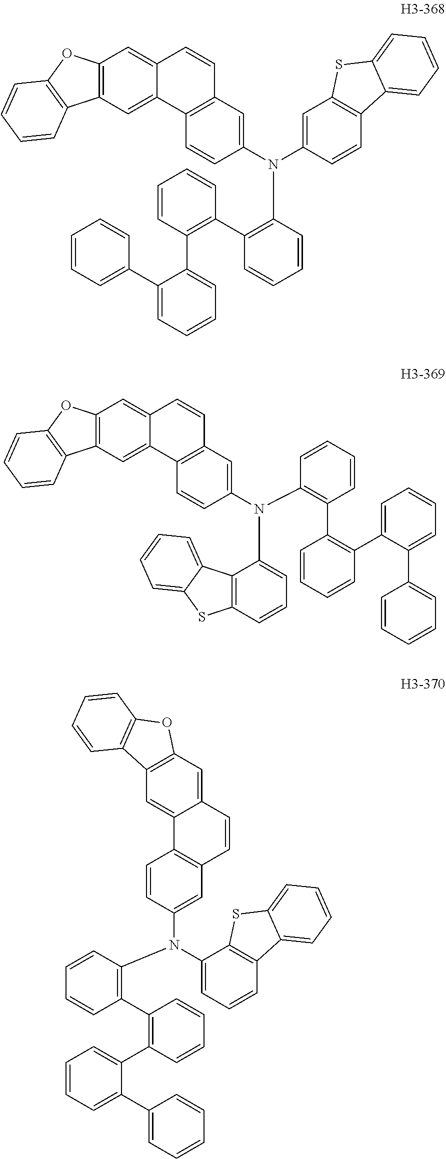 [SMILES: c1ccc(-c2ccccc2-c2ccccc2-c2ccccc2N(c2ccc3c(ccc4cc5oc6ccccc6c5cc43)c2)c2ccc3c(c2)sc2ccccc23)cc1 ; c1ccc(-c2ccccc2-c2ccccc2-c2ccccc2N(c2ccc3c(ccc4cc5oc6ccccc6c5cc43)c2)c2cccc3sc4ccccc4c23)cc1 ; c1ccc(-c2ccccc2-c2ccccc2-c2ccccc2N(c2ccc3c(ccc4cc5oc6ccccc6c5cc43)c2)c2cccc3c2sc2ccccc23)cc1] 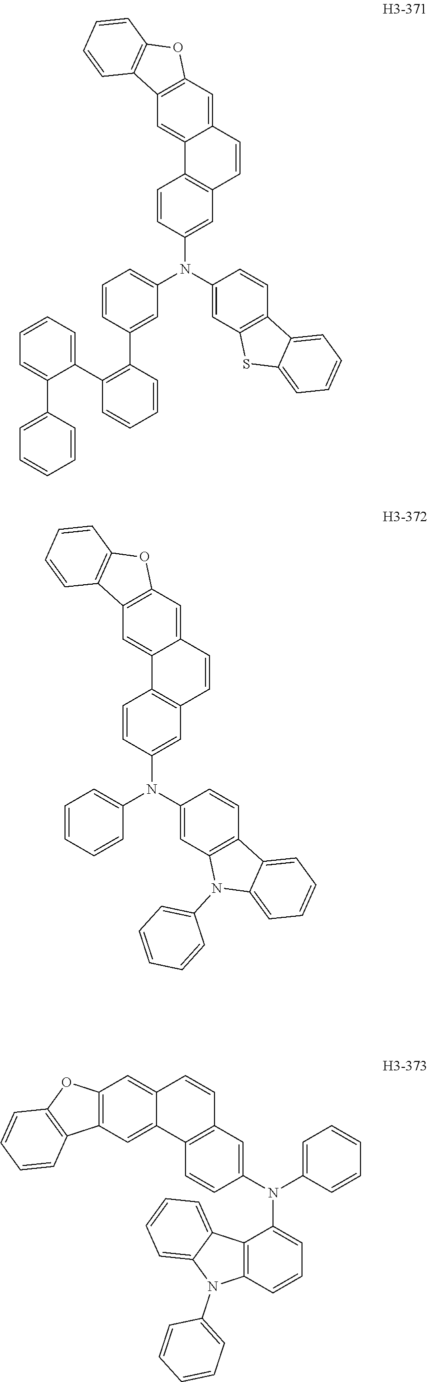 [SMILES: c1ccc(-c2ccccc2-c2ccccc2-c2cccc(N(c3ccc4c(ccc5cc6oc7ccccc7c6cc54)c3)c3ccc4c(c3)sc3ccccc34)c2)cc1 ; c1ccc(N(c2ccc3c(ccc4cc5oc6ccccc6c5cc43)c2)c2ccc3c4ccccc4n(-c4ccccc4)c3c2)cc1 ; c1ccc(N(c2ccc3c(ccc4cc5oc6ccccc6c5cc43)c2)c2cccc3c2c2ccccc2n3-c2ccccc2)cc1] 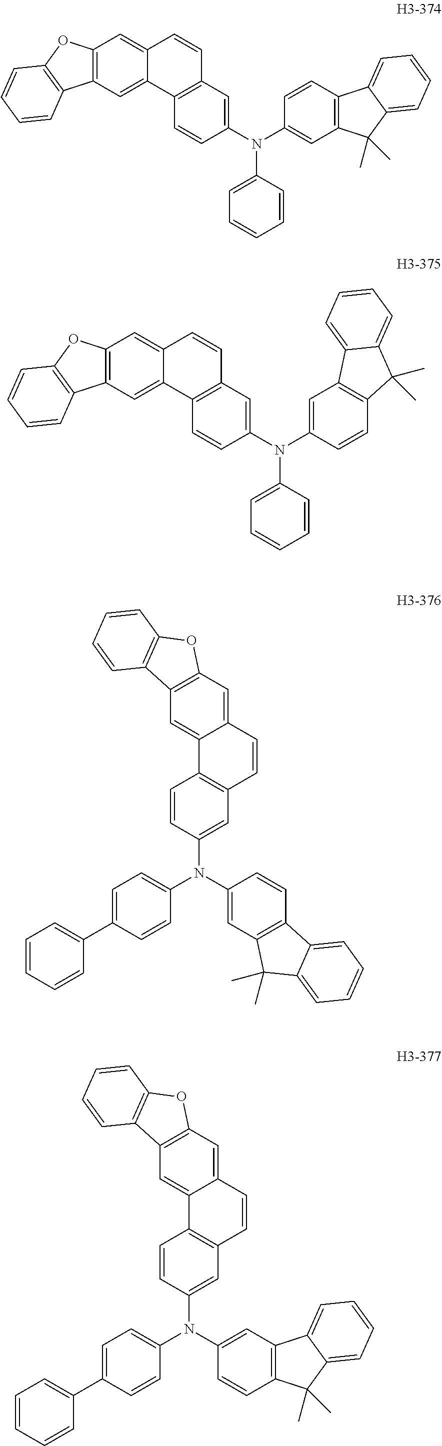 [SMILES: CC1(C)c2ccccc2-c2ccc(N(c3ccccc3)c3ccc4c(ccc5cc6oc7ccccc7c6cc54)c3)cc21 ; CC1(C)c2ccccc2-c2cc(N(c3ccccc3)c3ccc4c(ccc5cc6oc7ccccc7c6cc54)c3)ccc21 ; CC1(C)c2ccccc2-c2ccc(N(c3ccc(-c4ccccc4)cc3)c3ccc4c(ccc5cc6oc7ccccc7c6cc54)c3)cc21 ; CC1(C)c2ccccc2-c2cc(N(c3ccc(-c4ccccc4)cc3)c3ccc4c(ccc5cc6oc7ccccc7c6cc54)c3)ccc21] 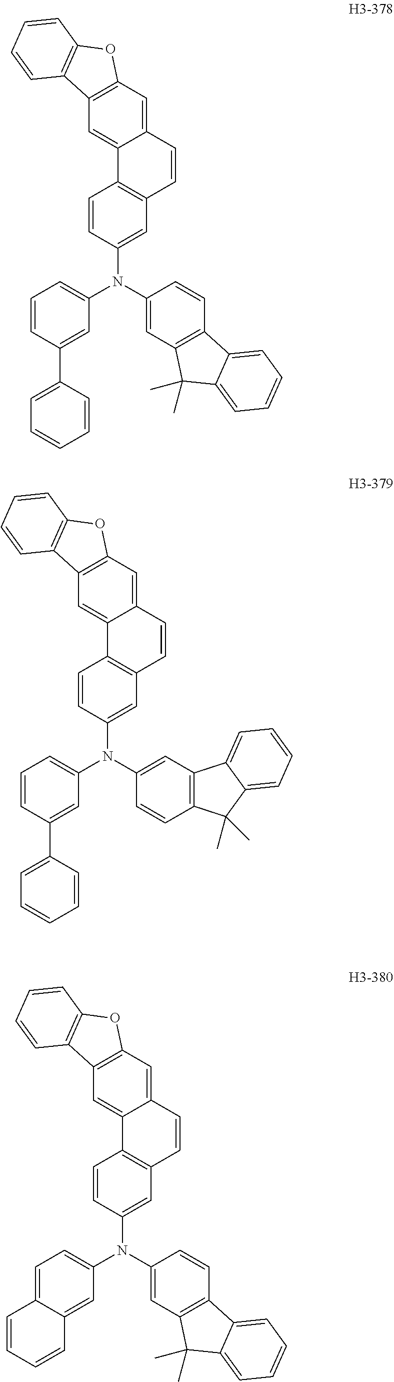 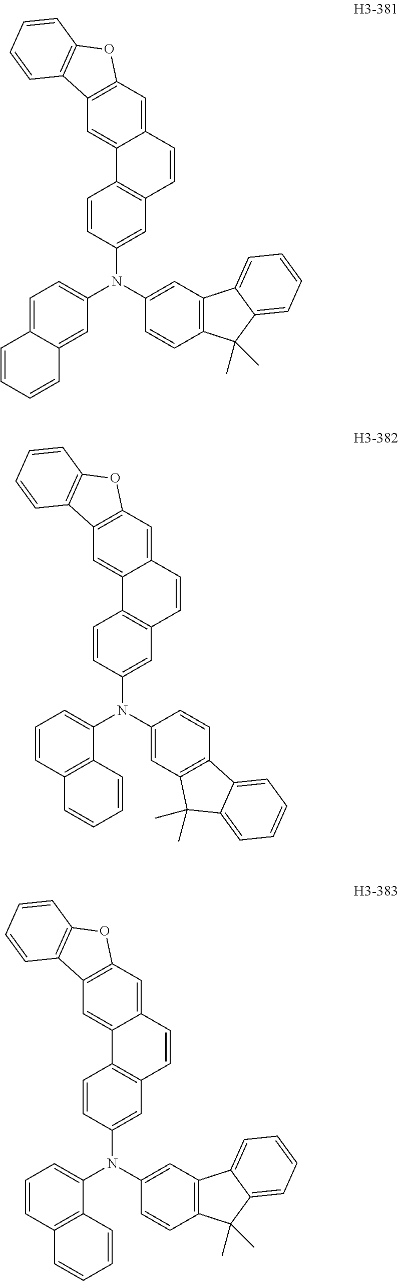 [SMILES: CC1(C)c2ccccc2-c2cc(N(c3ccc4ccccc4c3)c3ccc4c(ccc5cc6oc7ccccc7c6cc54)c3)ccc21 ; CC1(C)c2ccccc2-c2ccc(N(c3ccc4c(ccc5cc6oc7ccccc7c6cc54)c3)c3cccc4ccccc34)cc21 ; CC1(C)c2ccccc2-c2cc(N(c3ccc4c(ccc5cc6oc7ccccc7c6cc54)c3)c3cccc4ccccc34)ccc21] 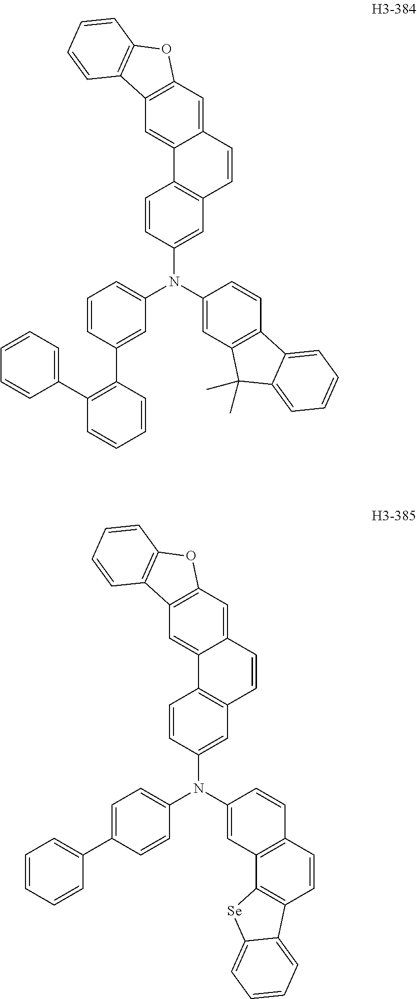 [SMILES: CC1(C)c2ccccc2-c2ccc(N(c3cccc(-c4ccccc4-c4ccccc4)c3)c3ccc4c(ccc5cc6oc7ccccc7c6cc54)c3)cc21 ; c1ccc(-c2ccc(N(c3ccc4c(ccc5cc6oc7ccccc7c6cc54)c3)c3ccc4ccc5c6ccccc6[se]c5c4c3)cc2)cc1] 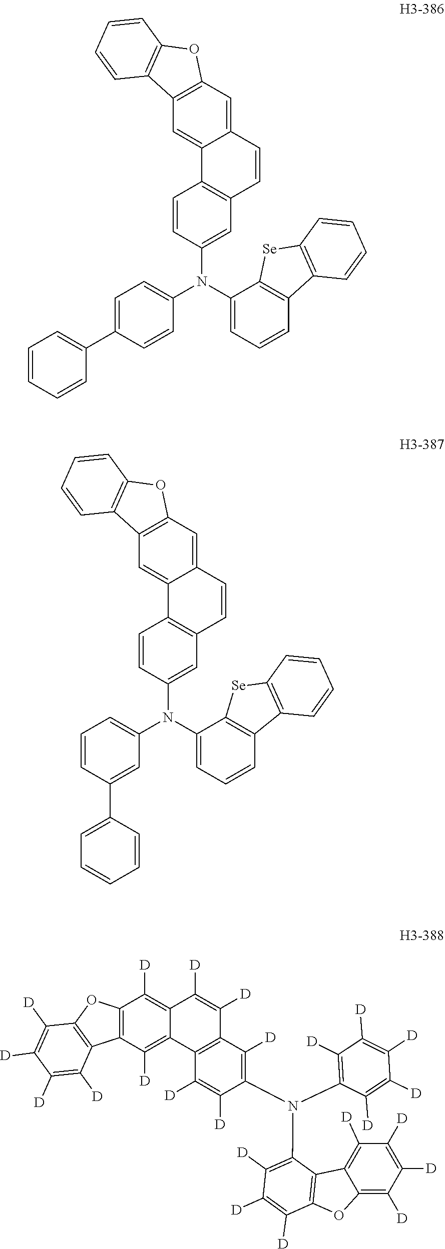 [SMILES: c1ccc(-c2ccc(N(c3ccc4c(ccc5cc6oc7ccccc7c6cc54)c3)c3cccc4c3[se]c3ccccc34)cc2)cc1 ; c1ccc(-c2cccc(N(c3ccc4c(ccc5cc6oc7ccccc7c6cc54)c3)c3cccc4c3[se]c3ccccc34)c2)cc1 ; [2H]c1c([2H])c([2H])c(N(c2c([2H])c([2H])c3c(c2[2H])c([2H])c([2H])c2c([2H])c4oc5c([2H])c([2H])c([2H])c([2H])c5c4c([2H])c23)c2c([2H])c([2H])c([2H])c3oc4c([2H])c([2H])c([2H])c([2H])c4c23)c([2H])c1[2H]] 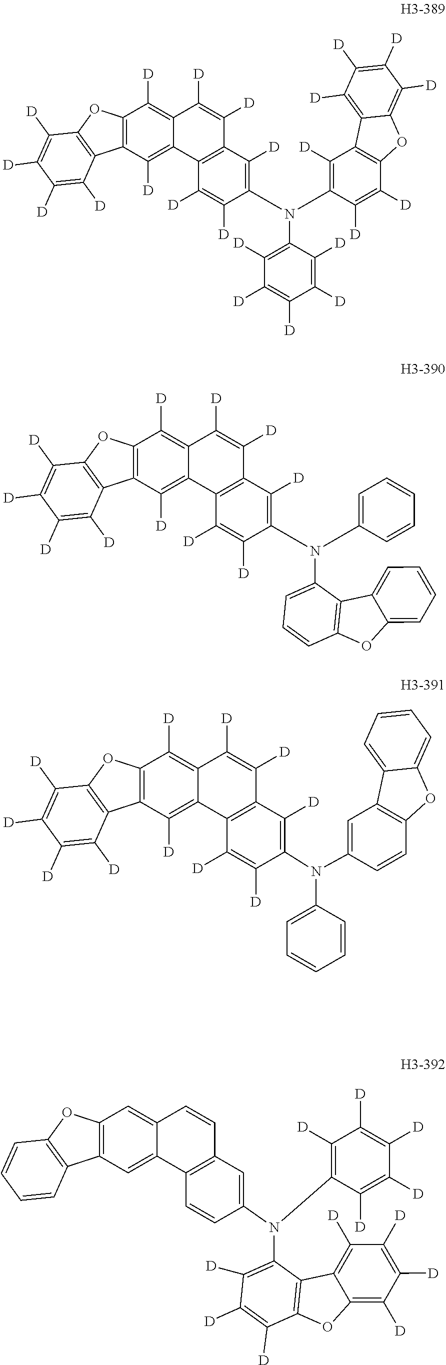 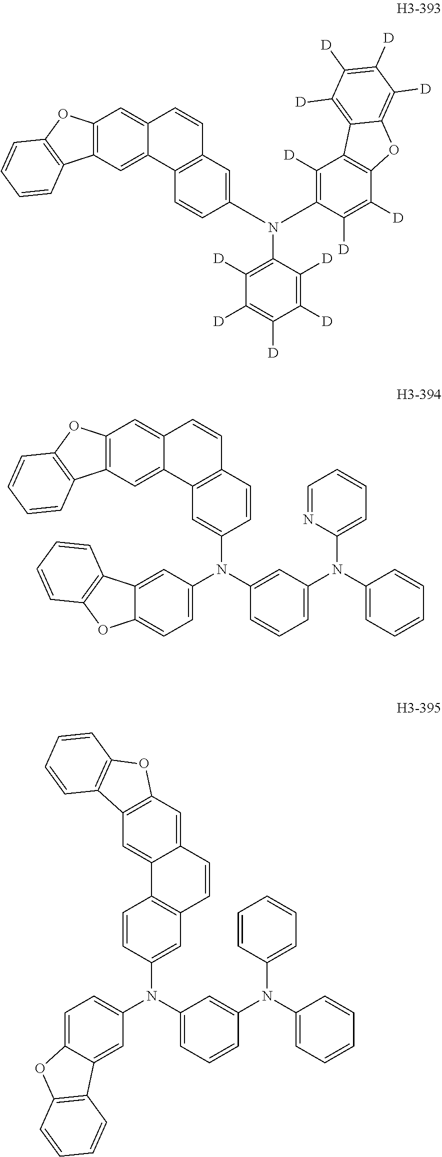 [SMILES: [2H]c1c([2H])c([2H])c(N(c2ccc3c(ccc4cc5oc6ccccc6c5cc43)c2)c2c([2H])c([2H])c3oc4c([2H])c([2H])c([2H])c([2H])c4c3c2[2H])c([2H])c1[2H] ; c1ccc(N(c2cccc(N(c3ccc4ccc5cc6oc7ccccc7c6cc5c4c3)c3ccc4oc5ccccc5c4c3)c2)c2ccccn2)cc1 ; c1ccc(N(c2ccccc2)c2cccc(N(c3ccc4c(ccc5cc6oc7ccccc7c6cc54)c3)c3ccc4oc5ccccc5c4c3)c2)cc1] 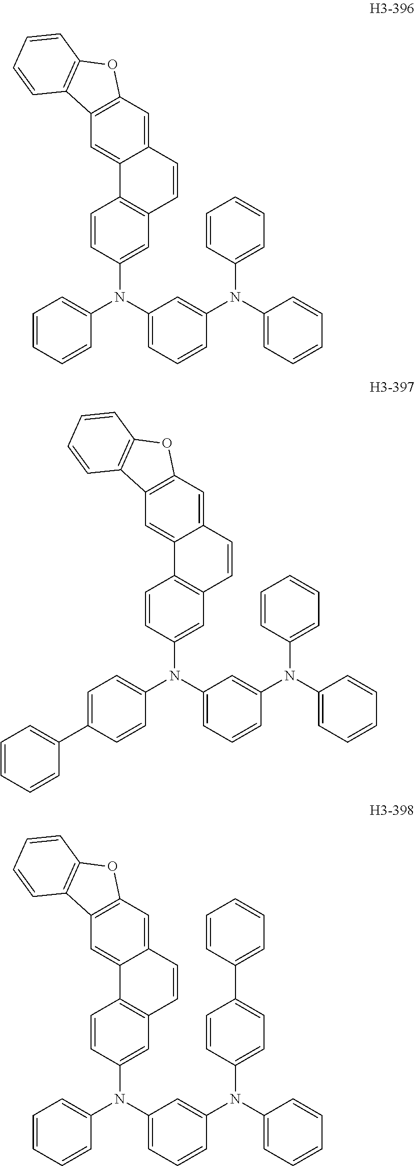 [SMILES: c1ccc(N(c2ccccc2)c2cccc(N(c3ccccc3)c3ccc4c(ccc5cc6oc7ccccc7c6cc54)c3)c2)cc1 ; c1ccc(-c2ccc(N(c3cccc(N(c4ccccc4)c4ccccc4)c3)c3ccc4c(ccc5cc6oc7ccccc7c6cc54)c3)cc2)cc1 ; c1ccc(-c2ccc(N(c3ccccc3)c3cccc(N(c4ccccc4)c4ccc5c(ccc6cc7oc8ccccc8c7cc65)c4)c3)cc2)cc1] 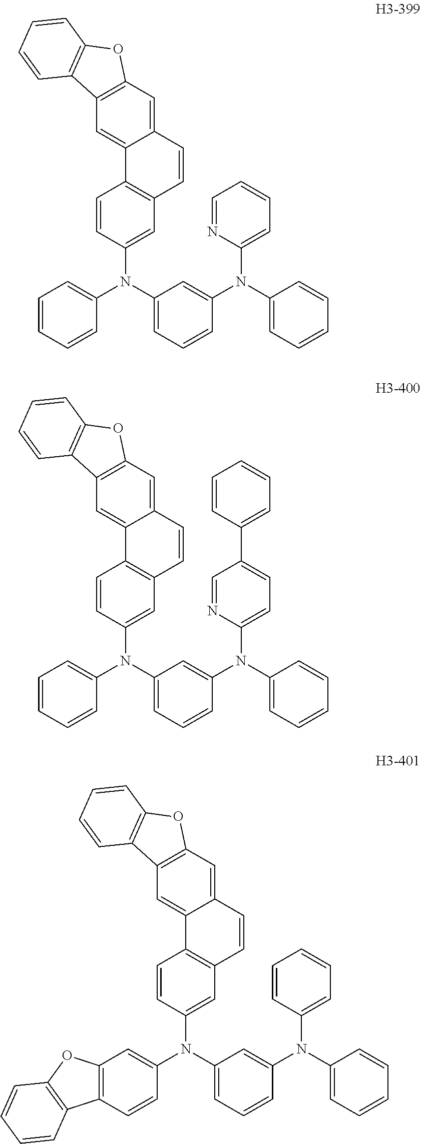 [SMILES: c1ccc(N(c2cccc(N(c3ccccc3)c3ccccn3)c2)c2ccc3c(ccc4cc5oc6ccccc6c5cc43)c2)cc1 ; c1ccc(-c2ccc(N(c3ccccc3)c3cccc(N(c4ccccc4)c4ccc5c(ccc6cc7oc8ccccc8c7cc65)c4)c3)nc2)cc1 ; c1ccc(N(c2ccccc2)c2cccc(N(c3ccc4c(ccc5cc6oc7ccccc7c6cc54)c3)c3ccc4c(c3)oc3ccccc34)c2)cc1] 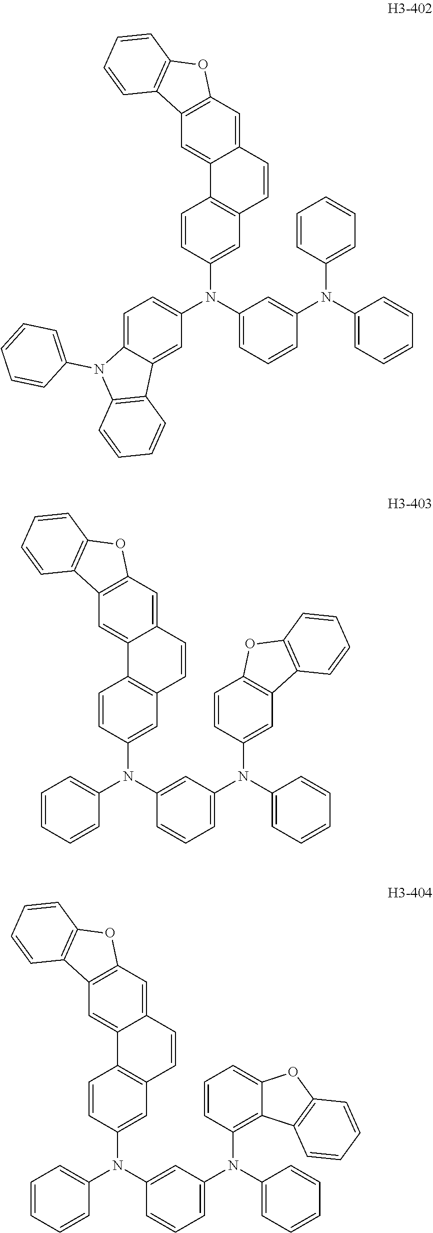 [SMILES: c1ccc(N(c2ccccc2)c2cccc(N(c3ccc4c(ccc5cc6oc7ccccc7c6cc54)c3)c3ccc4c(c3)c3ccccc3n4-c3ccccc3)c2)cc1 ; c1ccc(N(c2cccc(N(c3ccccc3)c3ccc4oc5ccccc5c4c3)c2)c2ccc3c(ccc4cc5oc6ccccc6c5cc43)c2)cc1 ; c1ccc(N(c2cccc(N(c3ccccc3)c3cccc4oc5ccccc5c34)c2)c2ccc3c(ccc4cc5oc6ccccc6c5cc43)c2)cc1] 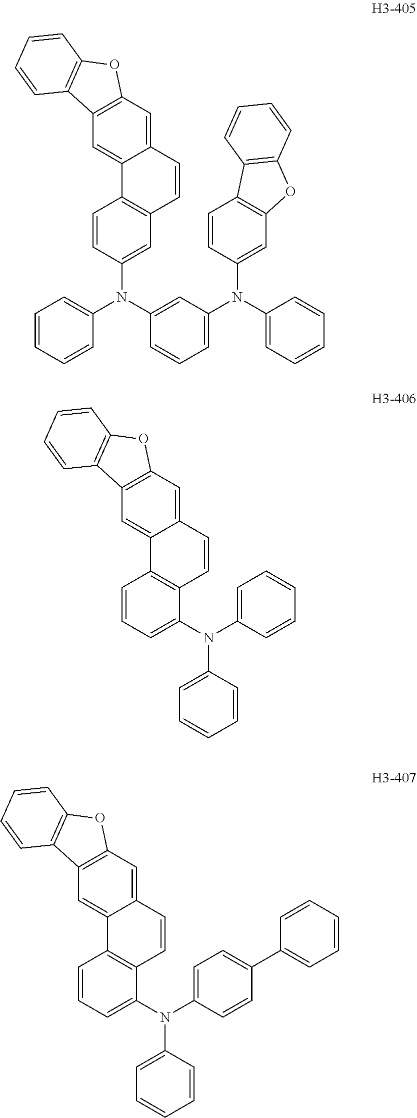 [SMILES: c1ccc(N(c2cccc(N(c3ccccc3)c3ccc4c(c3)oc3ccccc34)c2)c2ccc3c(ccc4cc5oc6ccccc6c5cc43)c2)cc1 ; c1ccc(N(c2ccccc2)c2cccc3c2ccc2cc4oc5ccccc5c4cc23)cc1 ; c1ccc(-c2ccc(N(c3ccccc3)c3cccc4c3ccc3cc5oc6ccccc6c5cc34)cc2)cc1] 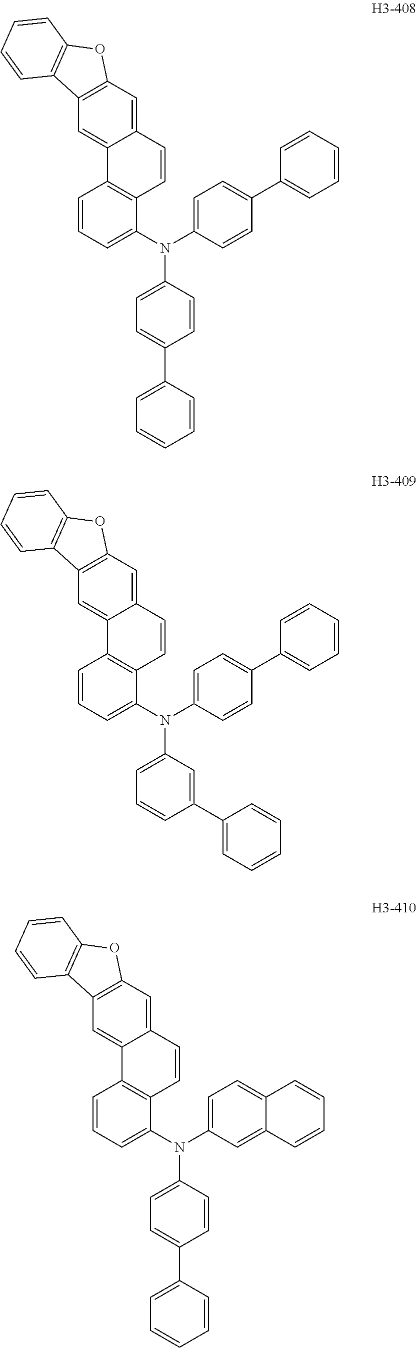 [SMILES: c1ccc(-c2ccc(N(c3ccc(-c4ccccc4)cc3)c3cccc4c3ccc3cc5oc6ccccc6c5cc34)cc2)cc1 ; c1ccc(-c2ccc(N(c3cccc(-c4ccccc4)c3)c3cccc4c3ccc3cc5oc6ccccc6c5cc34)cc2)cc1 ; c1ccc(-c2ccc(N(c3ccc4ccccc4c3)c3cccc4c3ccc3cc5oc6ccccc6c5cc34)cc2)cc1] 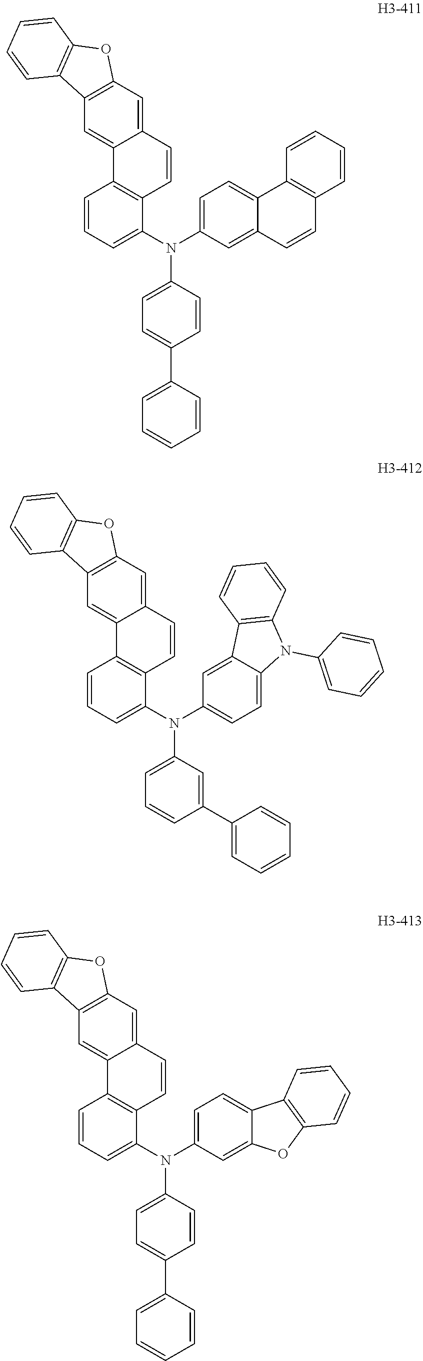 [SMILES: c1ccc(-c2ccc(N(c3ccc4c(ccc5ccccc54)c3)c3cccc4c3ccc3cc5oc6ccccc6c5cc34)cc2)cc1 ; c1ccc(-c2cccc(N(c3ccc4c(c3)c3ccccc3n4-c3ccccc3)c3cccc4c3ccc3cc5oc6ccccc6c5cc34)c2)cc1 ; c1ccc(-c2ccc(N(c3ccc4c(c3)oc3ccccc34)c3cccc4c3ccc3cc5oc6ccccc6c5cc34)cc2)cc1] 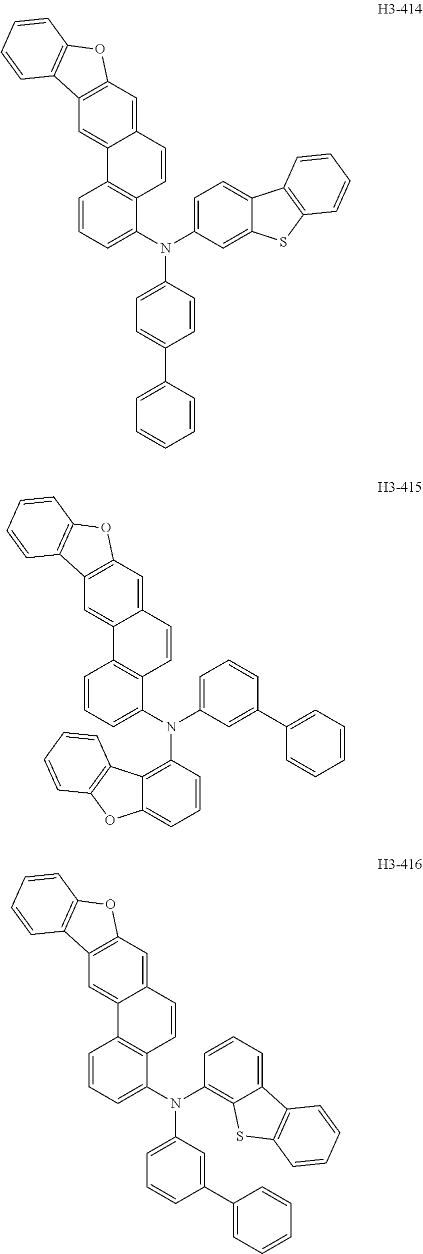 [SMILES: c1ccc(-c2ccc(N(c3ccc4c(c3)sc3ccccc34)c3cccc4c3ccc3cc5oc6ccccc6c5cc34)cc2)cc1 ; c1ccc(-c2cccc(N(c3cccc4c3ccc3cc5oc6ccccc6c5cc34)c3cccc4oc5ccccc5c34)c2)cc1 ; c1ccc(-c2cccc(N(c3cccc4c3ccc3cc5oc6ccccc6c5cc34)c3cccc4c3sc3ccccc34)c2)cc1] 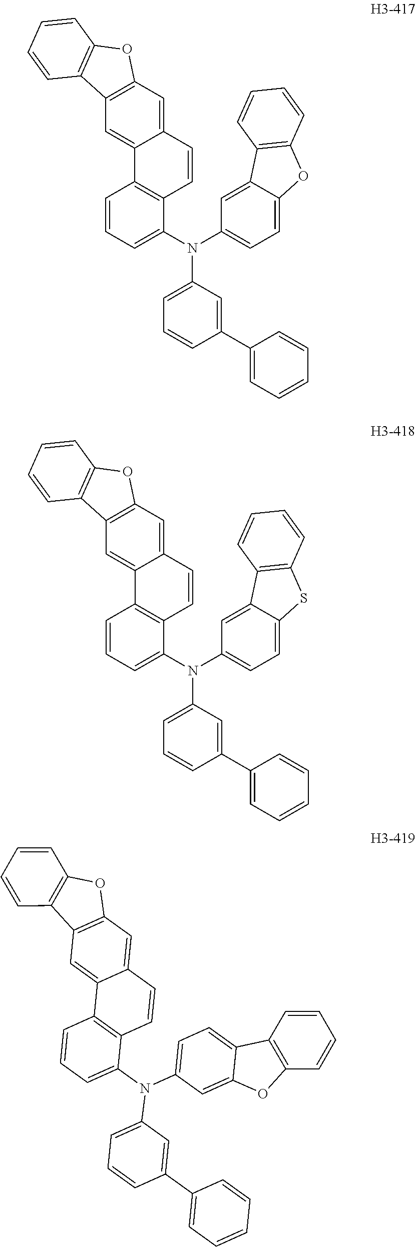 [SMILES: c1ccc(-c2cccc(N(c3ccc4oc5ccccc5c4c3)c3cccc4c3ccc3cc5oc6ccccc6c5cc34)c2)cc1 ; c1ccc(-c2cccc(N(c3ccc4sc5ccccc5c4c3)c3cccc4c3ccc3cc5oc6ccccc6c5cc34)c2)cc1 ; c1ccc(-c2cccc(N(c3ccc4c(c3)oc3ccccc34)c3cccc4c3ccc3cc5oc6ccccc6c5cc34)c2)cc1] 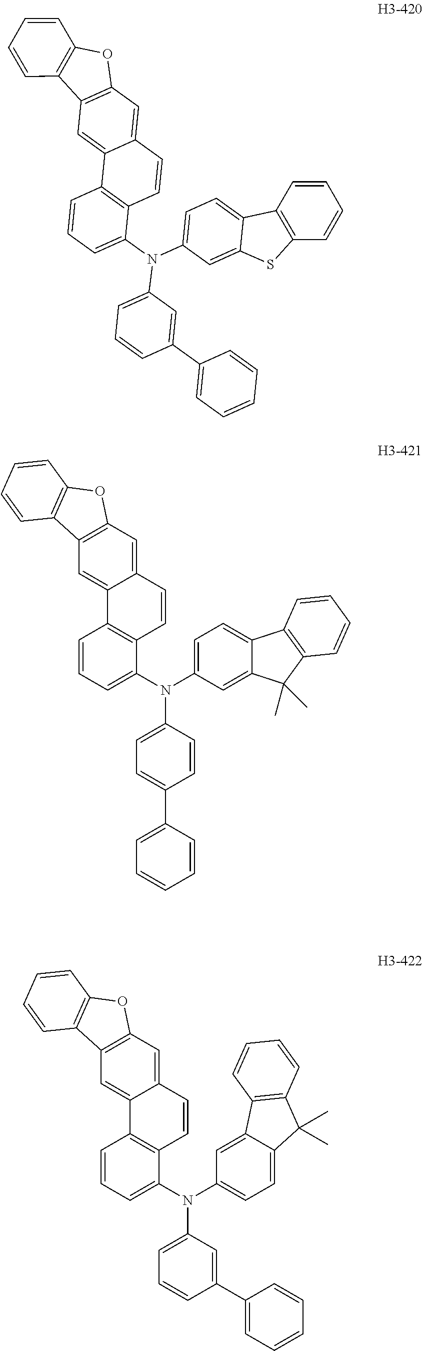 [SMILES: c1ccc(-c2cccc(N(c3ccc4c(c3)sc3ccccc34)c3cccc4c3ccc3cc5oc6ccccc6c5cc34)c2)cc1 ; CC1(C)c2ccccc2-c2ccc(N(c3ccc(-c4ccccc4)cc3)c3cccc4c3ccc3cc5oc6ccccc6c5cc34)cc21 ; CC1(C)c2ccccc2-c2cc(N(c3cccc(-c4ccccc4)c3)c3cccc4c3ccc3cc5oc6ccccc6c5cc34)ccc21] 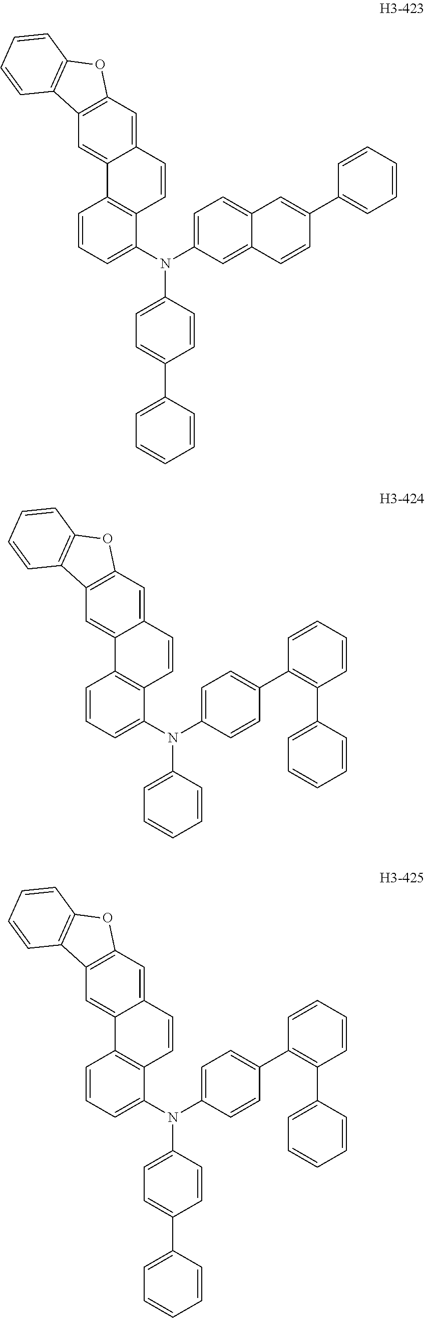 [SMILES: c1ccc(-c2ccc(N(c3ccc4cc(-c5ccccc5)ccc4c3)c3cccc4c3ccc3cc5oc6ccccc6c5cc34)cc2)cc1 ; c1ccc(-c2ccccc2-c2ccc(N(c3ccccc3)c3cccc4c3ccc3cc5oc6ccccc6c5cc34)cc2)cc1 ; c1ccc(-c2ccc(N(c3ccc(-c4ccccc4-c4ccccc4)cc3)c3cccc4c3ccc3cc5oc6ccccc6c5cc34)cc2)cc1] 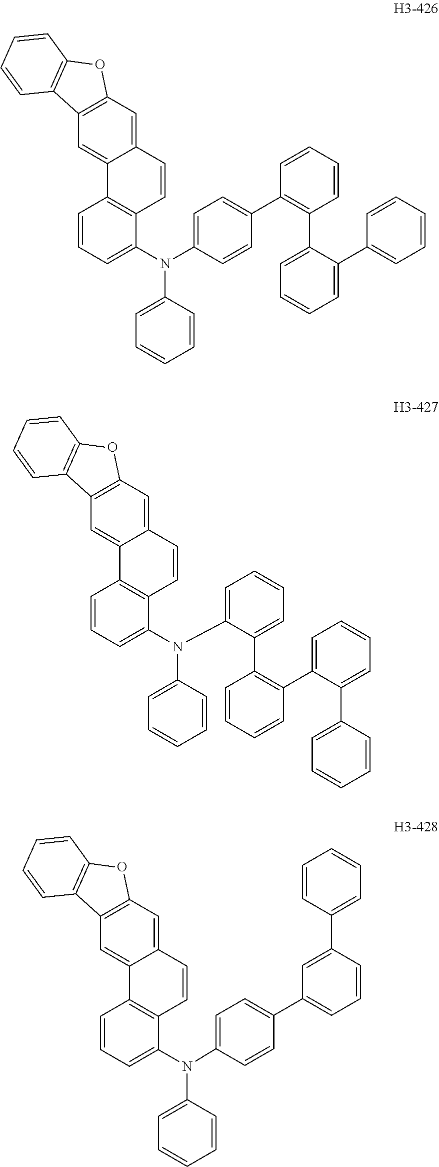 [SMILES: c1ccc(-c2ccccc2-c2ccccc2-c2ccc(N(c3ccccc3)c3cccc4c3ccc3cc5oc6ccccc6c5cc34)cc2)cc1 ; c1ccc(-c2ccccc2-c2ccccc2-c2ccccc2N(c2ccccc2)c2cccc3c2ccc2cc4oc5ccccc5c4cc23)cc1 ; c1ccc(-c2cccc(-c3ccc(N(c4ccccc4)c4cccc5c4ccc4cc6oc7ccccc7c6cc45)cc3)c2)cc1] 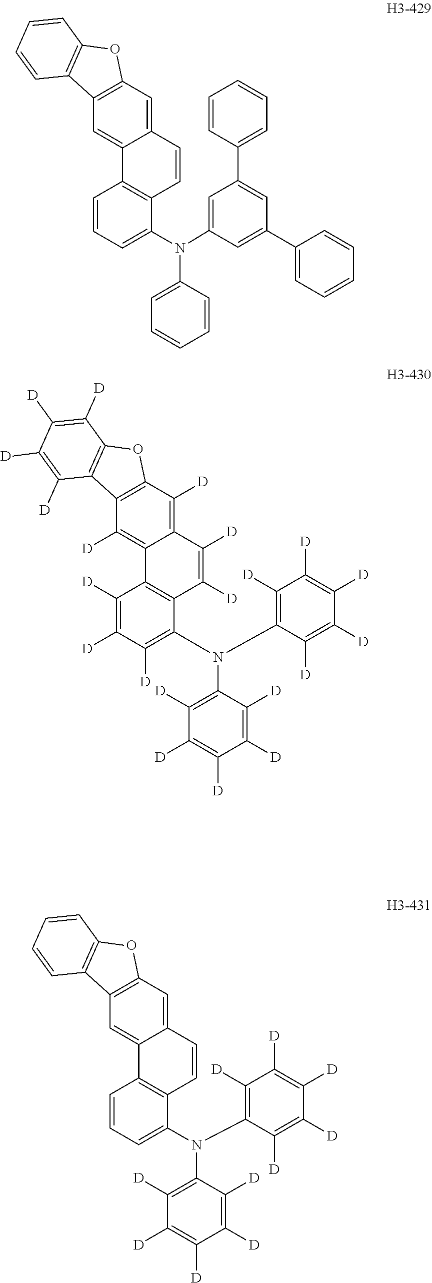 [SMILES: c1ccc(-c2cc(-c3ccccc3)cc(N(c3ccccc3)c3cccc4c3ccc3cc5oc6ccccc6c5cc34)c2)cc1 ; [2H]c1c([2H])c([2H])c(N(c2c([2H])c([2H])c([2H])c([2H])c2[2H])c2c([2H])c([2H])c([2H])c3c2c([2H])c([2H])c2c([2H])c4oc5c([2H])c([2H])c([2H])c([2H])c5c4c([2H])c23)c([2H])c1[2H] ; [2H]c1c([2H])c([2H])c(N(c2c([2H])c([2H])c([2H])c([2H])c2[2H])c2cccc3c2ccc2cc4oc5ccccc5c4cc23)c([2H])c1[2H]] 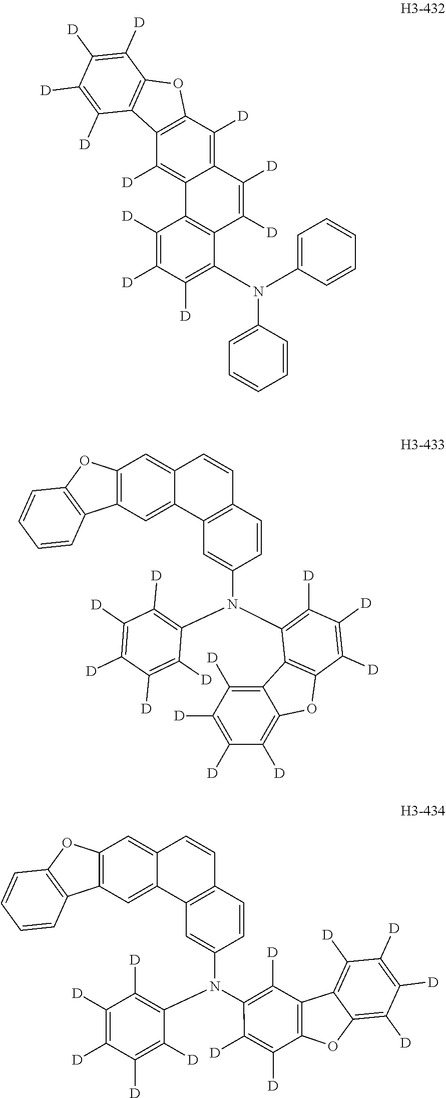 [SMILES: [2H]c1c([2H])c([2H])c2c(oc3c([2H])c4c([2H])c([2H])c5c(N(c6ccccc6)c6ccccc6)c([2H])c([2H])c([2H])c5c4c([2H])c32)c1[2H] ; [2H]c1c([2H])c([2H])c(N(c2ccc3ccc4cc5oc6ccccc6c5cc4c3c2)c2c([2H])c([2H])c([2H])c3oc4c([2H])c([2H])c([2H])c([2H])c4c23)c([2H])c1[2H] ; [2H]c1c([2H])c([2H])c(N(c2ccc3ccc4cc5oc6ccccc6c5cc4c3c2)c2c([2H])c([2H])c3oc4c([2H])c([2H])c([2H])c([2H])c4c3c2[2H])c([2H])c1[2H]] 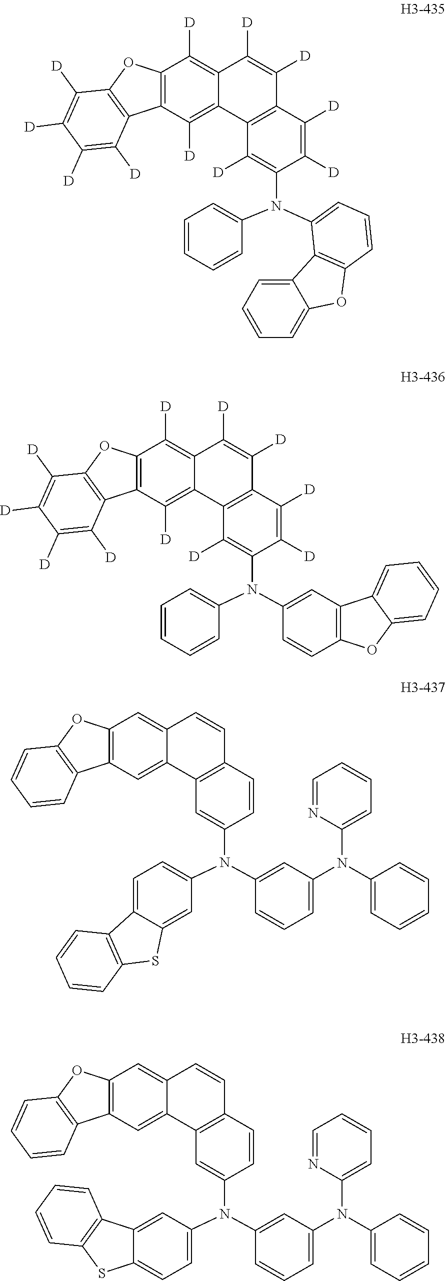 [SMILES: [2H]c1c([2H])c([2H])c2c(oc3c([2H])c4c([2H])c([2H])c5c([2H])c([2H])c(N(c6ccccc6)c6cccc7oc8ccccc8c67)c([2H])c5c4c([2H])c32)c1[2H] ; [2H]c1c([2H])c([2H])c2c(oc3c([2H])c4c([2H])c([2H])c5c([2H])c([2H])c(N(c6ccccc6)c6ccc7oc8ccccc8c7c6)c([2H])c5c4c([2H])c32)c1[2H] ; c1ccc(N(c2cccc(N(c3ccc4c(c3)sc3ccccc34)c3ccc4ccc5cc6oc7ccccc7c6cc5c4c3)c2)c2ccccn2)cc1 ; c1ccc(N(c2cccc(N(c3ccc4ccc5cc6oc7ccccc7c6cc5c4c3)c3ccc4sc5ccccc5c4c3)c2)c2ccccn2)cc1] 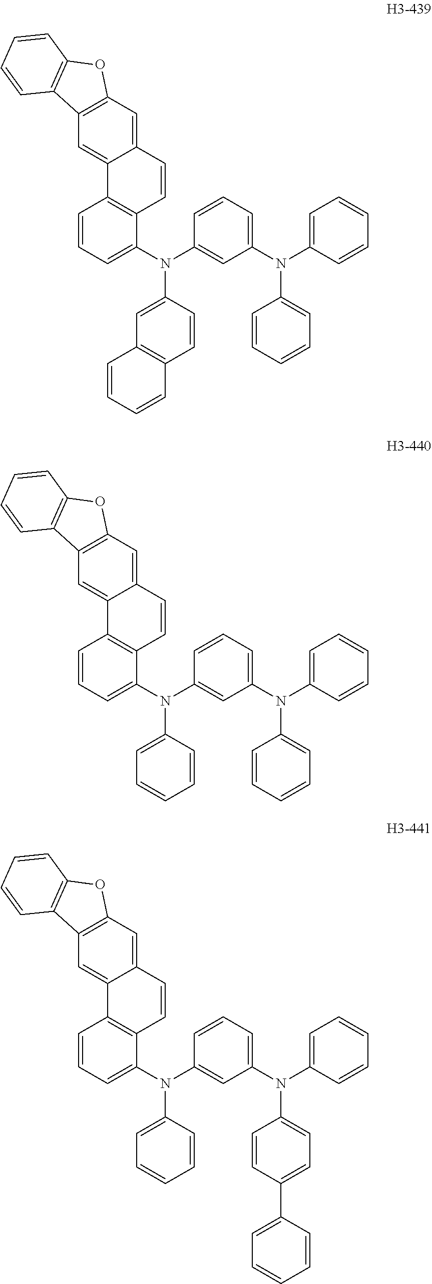 [SMILES: c1ccc(N(c2ccccc2)c2cccc(N(c3ccc4ccccc4c3)c3cccc4c3ccc3cc5oc6ccccc6c5cc34)c2)cc1 ; c1ccc(N(c2ccccc2)c2cccc(N(c3ccccc3)c3cccc4c3ccc3cc5oc6ccccc6c5cc34)c2)cc1 ; c1ccc(-c2ccc(N(c3ccccc3)c3cccc(N(c4ccccc4)c4cccc5c4ccc4cc6oc7ccccc7c6cc45)c3)cc2)cc1] 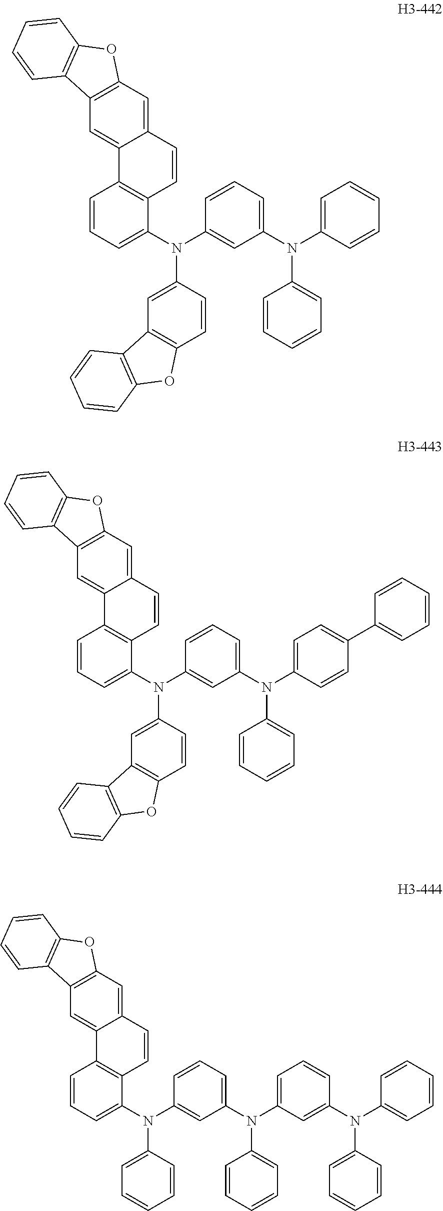 [SMILES: c1ccc(N(c2ccccc2)c2cccc(N(c3ccc4oc5ccccc5c4c3)c3cccc4c3ccc3cc5oc6ccccc6c5cc34)c2)cc1 ; c1ccc(-c2ccc(N(c3ccccc3)c3cccc(N(c4ccc5oc6ccccc6c5c4)c4cccc5c4ccc4cc6oc7ccccc7c6cc45)c3)cc2)cc1 ; c1ccc(N(c2ccccc2)c2cccc(N(c3ccccc3)c3cccc(N(c4ccccc4)c4cccc5c4ccc4cc6oc7ccccc7c6cc45)c3)c2)cc1] 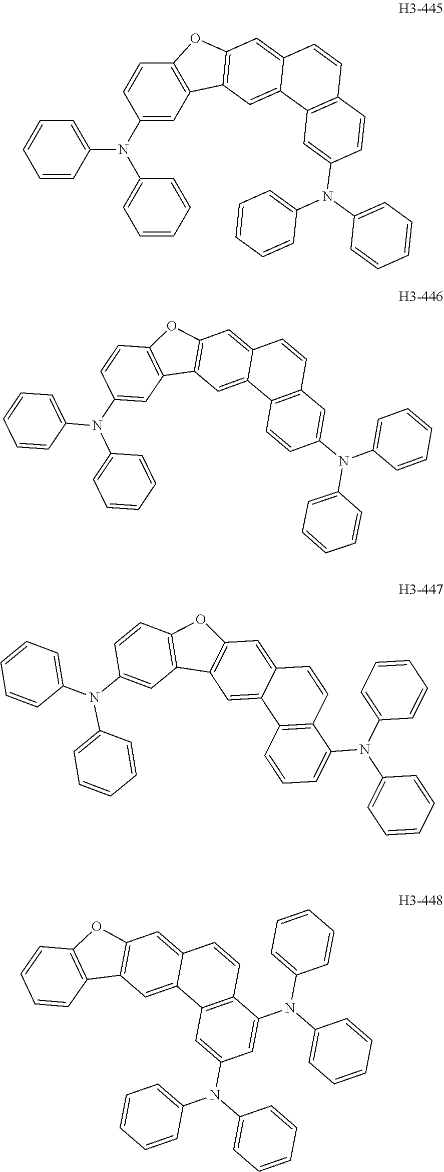 [SMILES: c1ccc(N(c2ccccc2)c2ccc3ccc4cc5oc6ccc(N(c7ccccc7)c7ccccc7)cc6c5cc4c3c2)cc1 ; c1ccc(N(c2ccccc2)c2ccc3c(ccc4cc5oc6ccc(N(c7ccccc7)c7ccccc7)cc6c5cc43)c2)cc1 ; c1ccc(N(c2ccccc2)c2ccc3oc4cc5ccc6c(N(c7ccccc7)c7ccccc7)cccc6c5cc4c3c2)cc1 ; c1ccc(N(c2ccccc2)c2cc(N(c3ccccc3)c3ccccc3)c3ccc4cc5oc6ccccc6c5cc4c3c2)cc1] 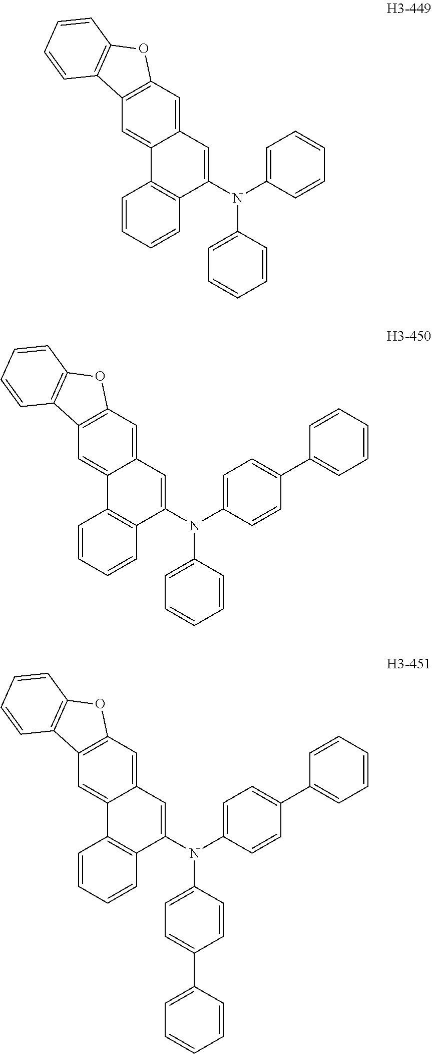 [SMILES: c1ccc(N(c2ccccc2)c2cc3cc4oc5ccccc5c4cc3c3ccccc23)cc1 ; c1ccc(-c2ccc(N(c3ccccc3)c3cc4cc5oc6ccccc6c5cc4c4ccccc34)cc2)cc1 ; c1ccc(-c2ccc(N(c3ccc(-c4ccccc4)cc3)c3cc4cc5oc6ccccc6c5cc4c4ccccc34)cc2)cc1] 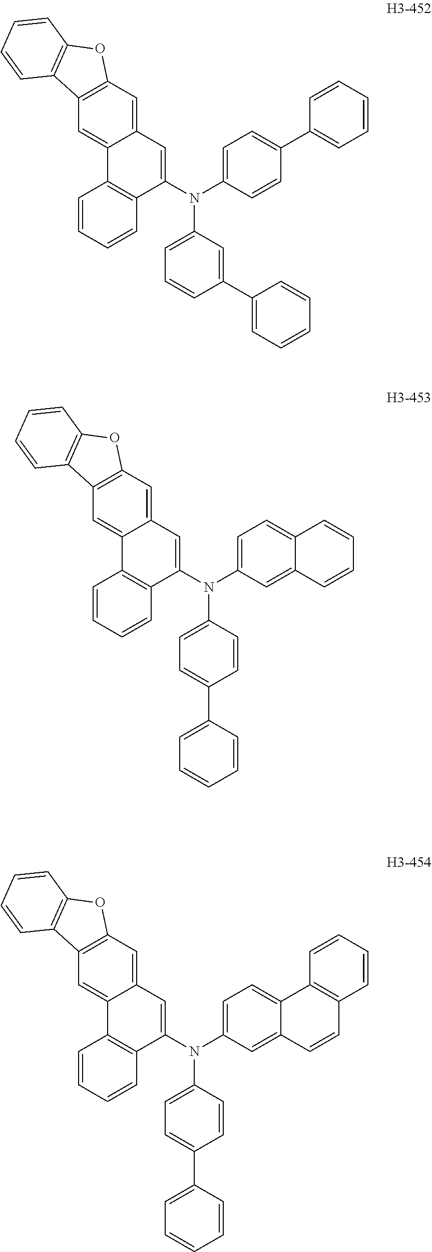 [SMILES: c1ccc(-c2ccc(N(c3cccc(-c4ccccc4)c3)c3cc4cc5oc6ccccc6c5cc4c4ccccc34)cc2)cc1 ; c1ccc(-c2ccc(N(c3ccc4ccccc4c3)c3cc4cc5oc6ccccc6c5cc4c4ccccc34)cc2)cc1 ; c1ccc(-c2ccc(N(c3ccc4c(ccc5ccccc54)c3)c3cc4cc5oc6ccccc6c5cc4c4ccccc34)cc2)cc1] 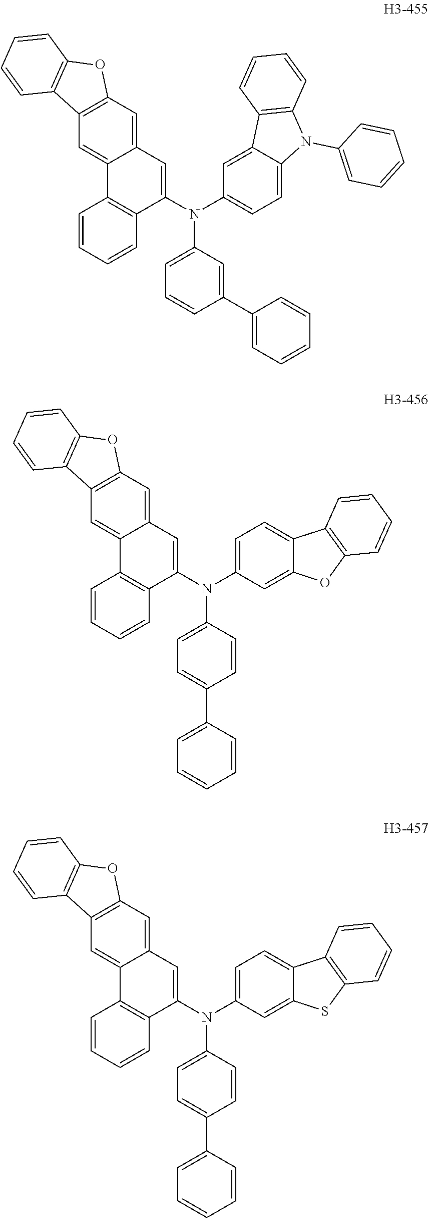 [SMILES: c1ccc(-c2cccc(N(c3ccc4c(c3)c3ccccc3n4-c3ccccc3)c3cc4cc5oc6ccccc6c5cc4c4ccccc34)c2)cc1 ; c1ccc(-c2ccc(N(c3ccc4c(c3)oc3ccccc34)c3cc4cc5oc6ccccc6c5cc4c4ccccc34)cc2)cc1 ; c1ccc(-c2ccc(N(c3ccc4c(c3)sc3ccccc34)c3cc4cc5oc6ccccc6c5cc4c4ccccc34)cc2)cc1] 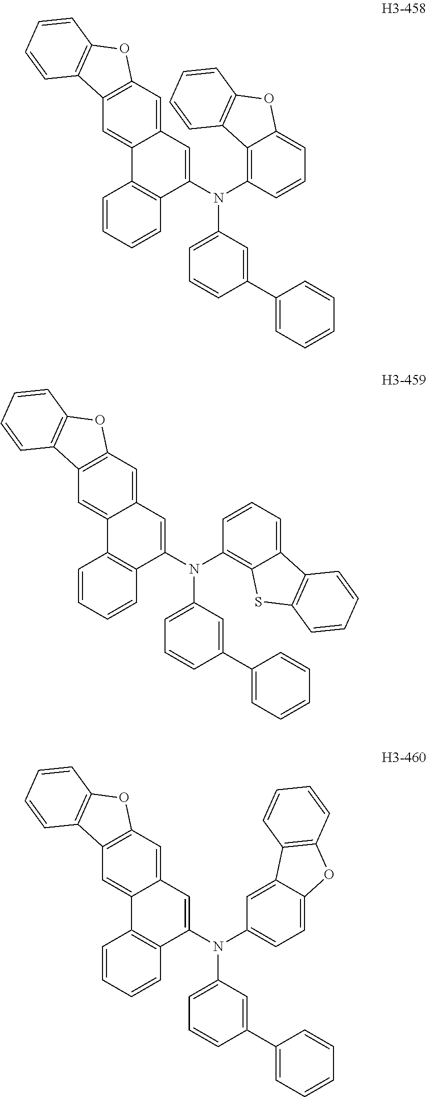 [SMILES: c1ccc(-c2cccc(N(c3cc4cc5oc6ccccc6c5cc4c4ccccc34)c3cccc4oc5ccccc5c34)c2)cc1 ; c1ccc(-c2cccc(N(c3cc4cc5oc6ccccc6c5cc4c4ccccc34)c3cccc4c3sc3ccccc34)c2)cc1 ; c1ccc(-c2cccc(N(c3ccc4oc5ccccc5c4c3)c3cc4cc5oc6ccccc6c5cc4c4ccccc34)c2)cc1] 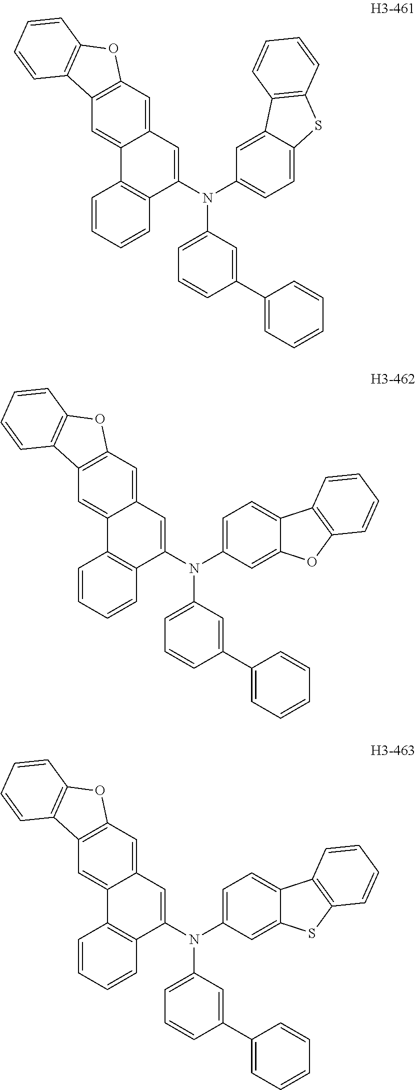 [SMILES: c1ccc(-c2cccc(N(c3ccc4sc5ccccc5c4c3)c3cc4cc5oc6ccccc6c5cc4c4ccccc34)c2)cc1 ; c1ccc(-c2cccc(N(c3ccc4c(c3)oc3ccccc34)c3cc4cc5oc6ccccc6c5cc4c4ccccc34)c2)cc1 ; c1ccc(-c2cccc(N(c3ccc4c(c3)sc3ccccc34)c3cc4cc5oc6ccccc6c5cc4c4ccccc34)c2)cc1] 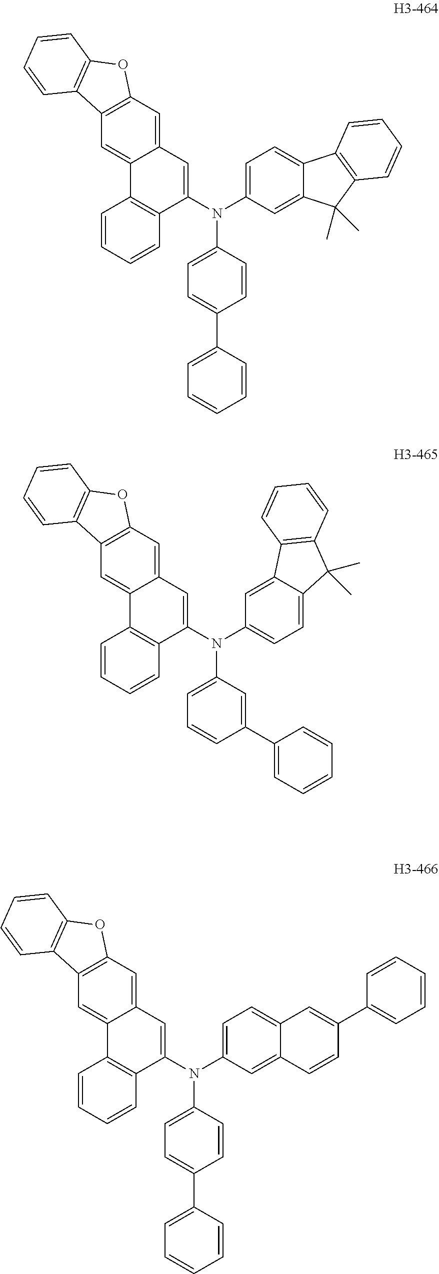 [SMILES: CC1(C)c2ccccc2-c2ccc(N(c3ccc(-c4ccccc4)cc3)c3cc4cc5oc6ccccc6c5cc4c4ccccc34)cc21 ; CC1(C)c2ccccc2-c2cc(N(c3cccc(-c4ccccc4)c3)c3cc4cc5oc6ccccc6c5cc4c4ccccc34)ccc21 ; c1ccc(-c2ccc(N(c3ccc4cc(-c5ccccc5)ccc4c3)c3cc4cc5oc6ccccc6c5cc4c4ccccc34)cc2)cc1] 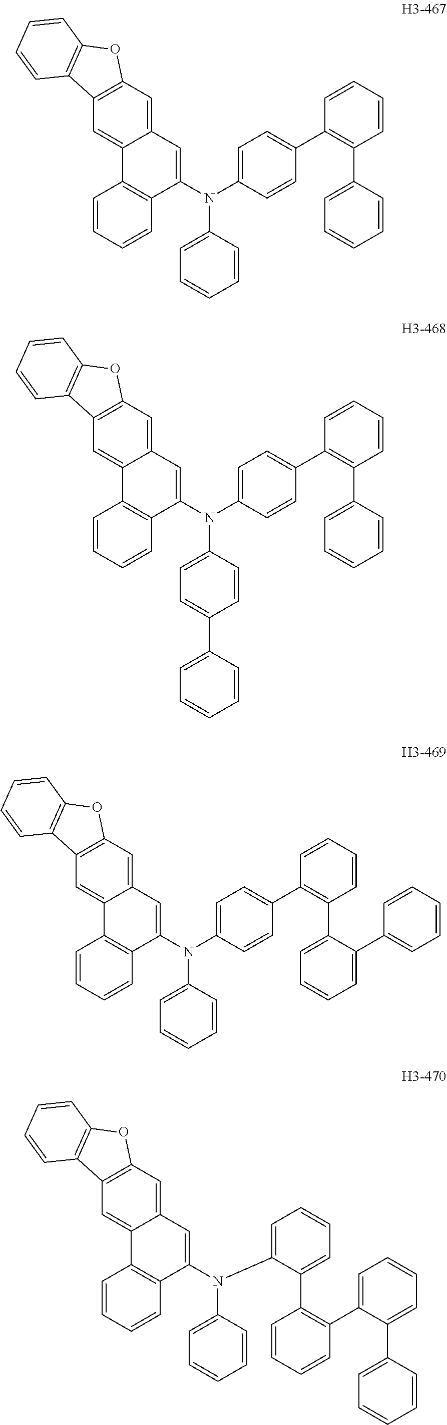 [SMILES: c1ccc(-c2ccccc2-c2ccc(N(c3ccccc3)c3cc4cc5oc6ccccc6c5cc4c4ccccc34)cc2)cc1 ; c1ccc(-c2ccc(N(c3ccc(-c4ccccc4-c4ccccc4)cc3)c3cc4cc5oc6ccccc6c5cc4c4ccccc34)cc2)cc1 ; c1ccc(-c2ccccc2-c2ccccc2-c2ccc(N(c3ccccc3)c3cc4cc5oc6ccccc6c5cc4c4ccccc34)cc2)cc1 ; c1ccc(-c2ccccc2-c2ccccc2-c2ccccc2N(c2ccccc2)c2cc3cc4oc5ccccc5c4cc3c3ccccc23)cc1] 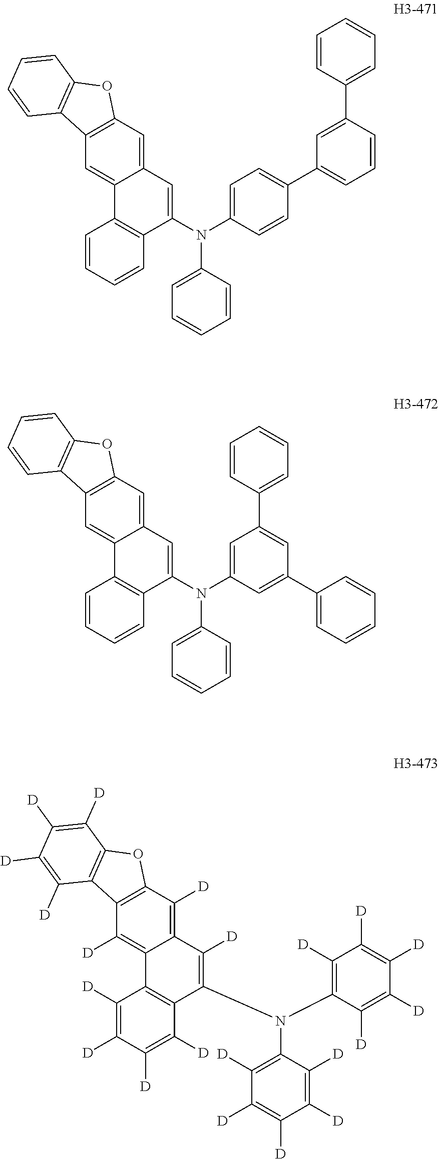 [SMILES: c1ccc(-c2cccc(-c3ccc(N(c4ccccc4)c4cc5cc6oc7ccccc7c6cc5c5ccccc45)cc3)c2)cc1 ; c1ccc(-c2cc(-c3ccccc3)cc(N(c3ccccc3)c3cc4cc5oc6ccccc6c5cc4c4ccccc34)c2)cc1 ; [2H]c1c([2H])c([2H])c(N(c2c([2H])c([2H])c([2H])c([2H])c2[2H])c2c([2H])c3c([2H])c4oc5c([2H])c([2H])c([2H])c([2H])c5c4c([2H])c3c3c([2H])c([2H])c([2H])c([2H])c23)c([2H])c1[2H]] 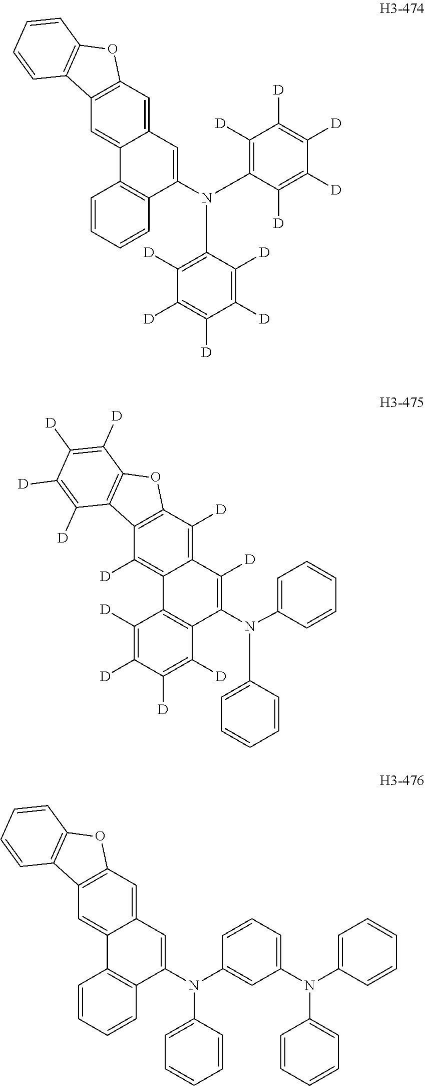 [SMILES: [2H]c1c([2H])c([2H])c(N(c2c([2H])c([2H])c([2H])c([2H])c2[2H])c2cc3cc4oc5ccccc5c4cc3c3ccccc23)c([2H])c1[2H] ; [2H]c1c([2H])c([2H])c2c(oc3c([2H])c4c([2H])c(N(c5ccccc5)c5ccccc5)c5c([2H])c([2H])c([2H])c([2H])c5c4c([2H])c32)c1[2H] ; c1ccc(N(c2ccccc2)c2cccc(N(c3ccccc3)c3cc4cc5oc6ccccc6c5cc4c4ccccc34)c2)cc1] 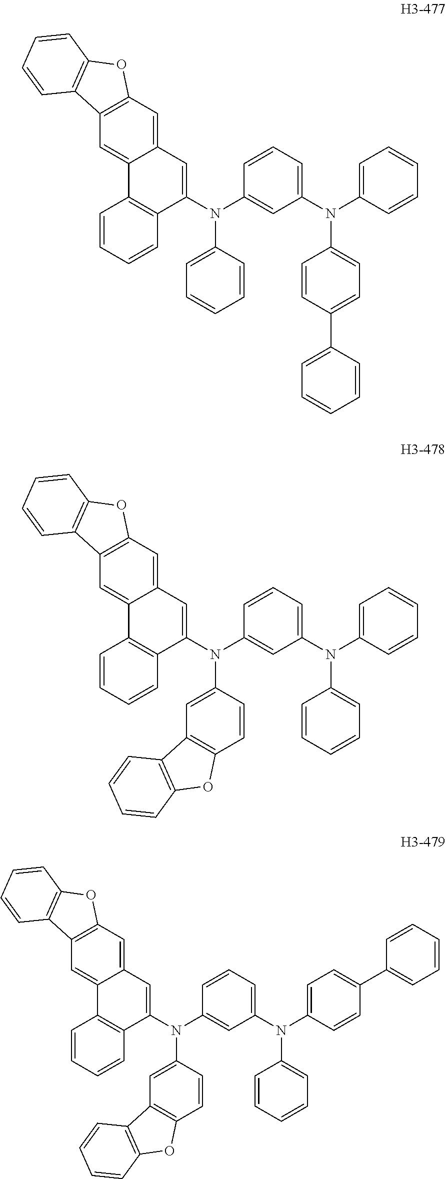 [SMILES: c1ccc(-c2ccc(N(c3ccccc3)c3cccc(N(c4ccccc4)c4cc5cc6oc7ccccc7c6cc5c5ccccc45)c3)cc2)cc1 ; c1ccc(N(c2ccccc2)c2cccc(N(c3ccc4oc5ccccc5c4c3)c3cc4cc5oc6ccccc6c5cc4c4ccccc34)c2)cc1 ; c1ccc(-c2ccc(N(c3ccccc3)c3cccc(N(c4ccc5oc6ccccc6c5c4)c4cc5cc6oc7ccccc7c6cc5c5ccccc45)c3)cc2)cc1] 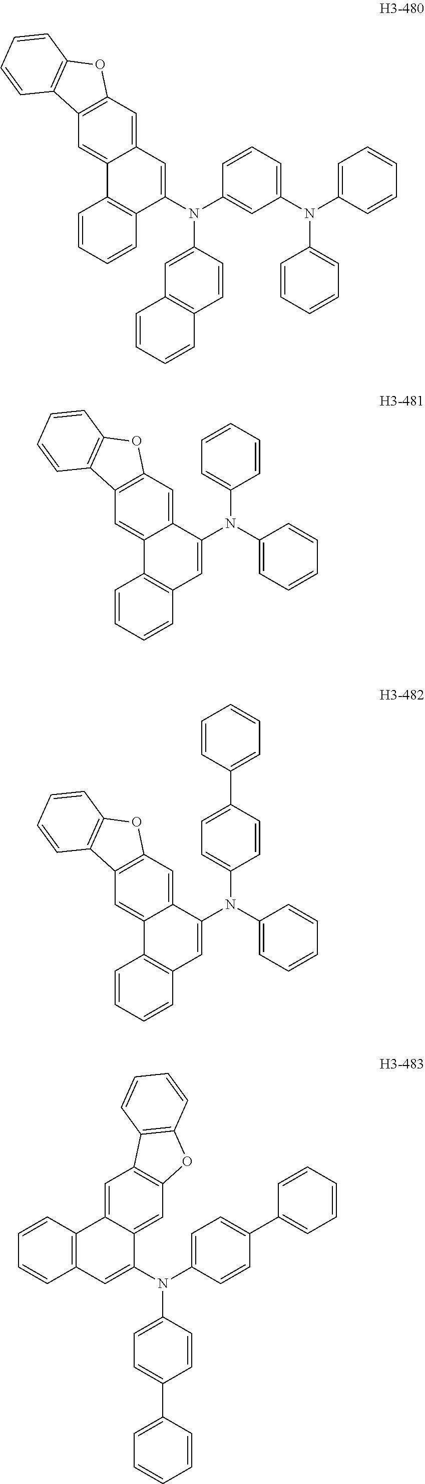 [SMILES: c1ccc(N(c2ccccc2)c2cccc(N(c3ccc4ccccc4c3)c3cc4cc5oc6ccccc6c5cc4c4ccccc34)c2)cc1 ; c1ccc(N(c2ccccc2)c2cc3ccccc3c3cc4c(cc23)oc2ccccc24)cc1 ; c1ccc(-c2ccc(N(c3ccccc3)c3cc4ccccc4c4cc5c(cc34)oc3ccccc35)cc2)cc1 ; c1ccc(-c2ccc(N(c3ccc(-c4ccccc4)cc3)c3cc4ccccc4c4cc5c(cc34)oc3ccccc35)cc2)cc1] 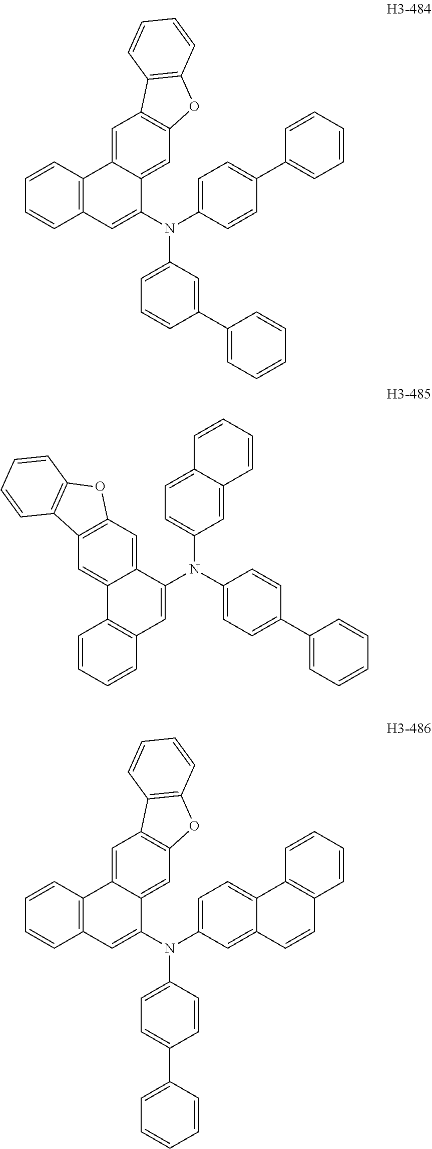 [SMILES: c1ccc(-c2ccc(N(c3cccc(-c4ccccc4)c3)c3cc4ccccc4c4cc5c(cc34)oc3ccccc35)cc2)cc1 ; c1ccc(-c2ccc(N(c3ccc4ccccc4c3)c3cc4ccccc4c4cc5c(cc34)oc3ccccc35)cc2)cc1 ; c1ccc(-c2ccc(N(c3ccc4c(ccc5ccccc54)c3)c3cc4ccccc4c4cc5c(cc34)oc3ccccc35)cc2)cc1] 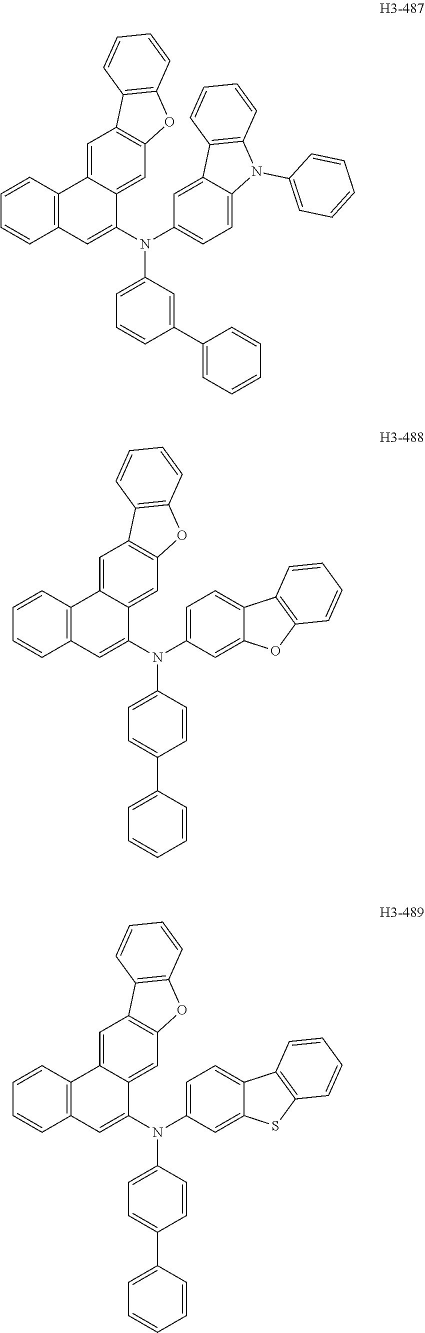 [SMILES: c1ccc(-c2cccc(N(c3ccc4c(c3)c3ccccc3n4-c3ccccc3)c3cc4ccccc4c4cc5c(cc34)oc3ccccc35)c2)cc1 ; c1ccc(-c2ccc(N(c3ccc4c(c3)oc3ccccc34)c3cc4ccccc4c4cc5c(cc34)oc3ccccc35)cc2)cc1 ; c1ccc(-c2ccc(N(c3ccc4c(c3)sc3ccccc34)c3cc4ccccc4c4cc5c(cc34)oc3ccccc35)cc2)cc1] 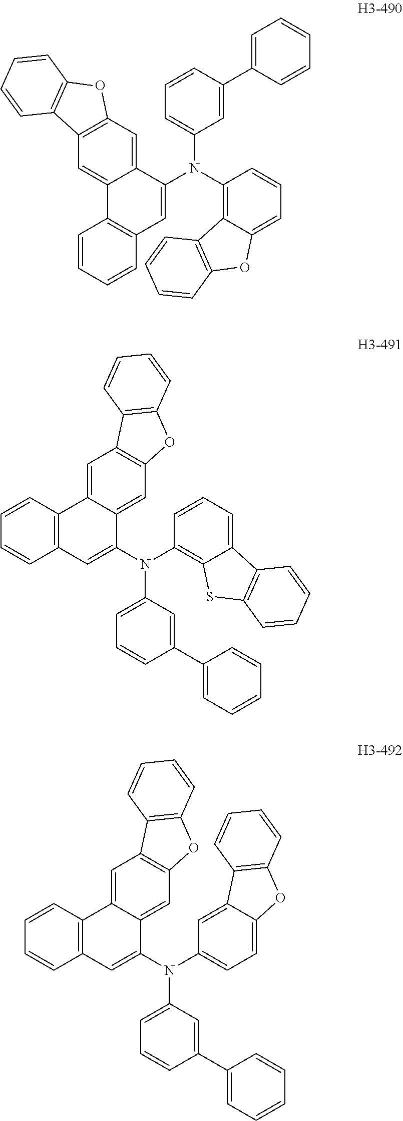 [SMILES: c1ccc(-c2cccc(N(c3cc4ccccc4c4cc5c(cc34)oc3ccccc35)c3cccc4oc5ccccc5c34)c2)cc1 ; c1ccc(-c2cccc(N(c3cc4ccccc4c4cc5c(cc34)oc3ccccc35)c3cccc4c3sc3ccccc34)c2)cc1 ; c1ccc(-c2cccc(N(c3ccc4oc5ccccc5c4c3)c3cc4ccccc4c4cc5c(cc34)oc3ccccc35)c2)cc1] 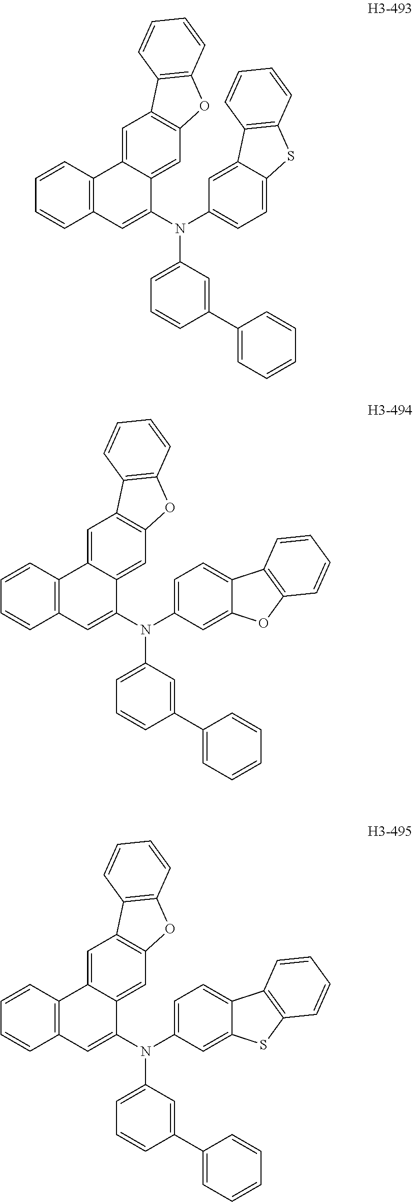 [SMILES: c1ccc(-c2cccc(N(c3ccc4sc5ccccc5c4c3)c3cc4ccccc4c4cc5c(cc34)oc3ccccc35)c2)cc1 ; c1ccc(-c2cccc(N(c3ccc4c(c3)oc3ccccc34)c3cc4ccccc4c4cc5c(cc34)oc3ccccc35)c2)cc1 ; c1ccc(-c2cccc(N(c3ccc4c(c3)sc3ccccc34)c3cc4ccccc4c4cc5c(cc34)oc3ccccc35)c2)cc1] 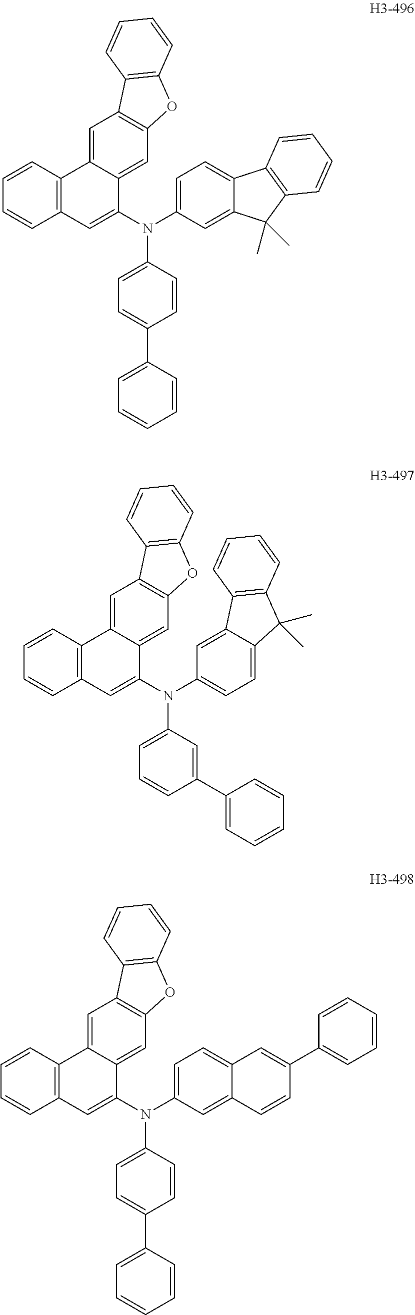 [SMILES: CC1(C)c2ccccc2-c2ccc(N(c3ccc(-c4ccccc4)cc3)c3cc4ccccc4c4cc5c(cc34)oc3ccccc35)cc21 ; CC1(C)c2ccccc2-c2cc(N(c3cccc(-c4ccccc4)c3)c3cc4ccccc4c4cc5c(cc34)oc3ccccc35)ccc21 ; c1ccc(-c2ccc(N(c3ccc4cc(-c5ccccc5)ccc4c3)c3cc4ccccc4c4cc5c(cc34)oc3ccccc35)cc2)cc1] 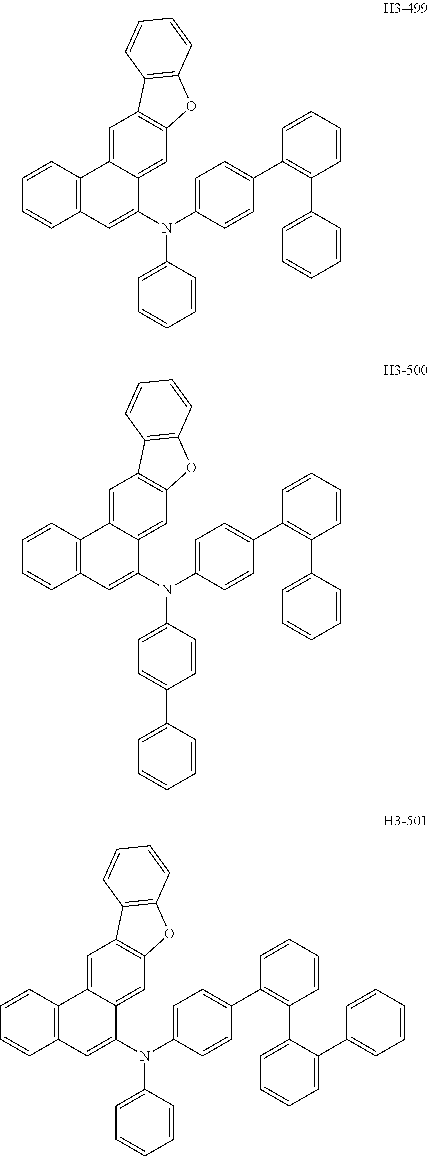 [SMILES: c1ccc(-c2ccccc2-c2ccc(N(c3ccccc3)c3cc4ccccc4c4cc5c(cc34)oc3ccccc35)cc2)cc1 ; c1ccc(-c2ccc(N(c3ccc(-c4ccccc4-c4ccccc4)cc3)c3cc4ccccc4c4cc5c(cc34)oc3ccccc35)cc2)cc1 ; c1ccc(-c2ccccc2-c2ccccc2-c2ccc(N(c3ccccc3)c3cc4ccccc4c4cc5c(cc34)oc3ccccc35)cc2)cc1] 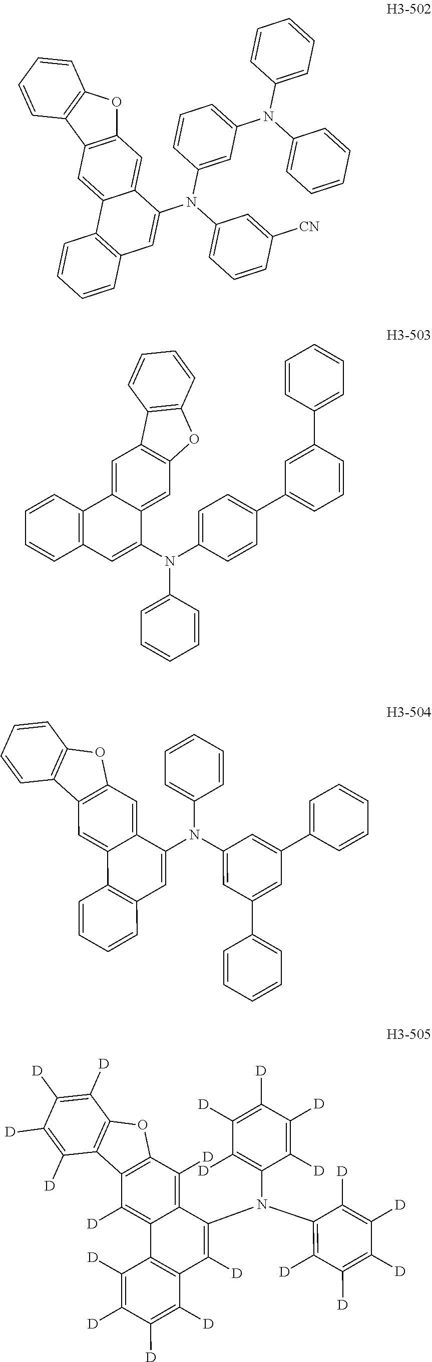 [SMILES: N#Cc1cccc(N(c2cccc(N(c3ccccc3)c3ccccc3)c2)c2cc3ccccc3c3cc4c(cc23)oc2ccccc24)c1 ; c1ccc(-c2cccc(-c3ccc(N(c4ccccc4)c4cc5ccccc5c5cc6c(cc45)oc4ccccc46)cc3)c2)cc1 ; c1ccc(-c2cc(-c3ccccc3)cc(N(c3ccccc3)c3cc4ccccc4c4cc5c(cc34)oc3ccccc35)c2)cc1 ; [2H]c1c([2H])c([2H])c(N(c2c([2H])c([2H])c([2H])c([2H])c2[2H])c2c([2H])c3c([2H])c([2H])c([2H])c([2H])c3c3c([2H])c4c(oc5c([2H])c([2H])c([2H])c([2H])c54)c([2H])c23)c([2H])c1[2H]] 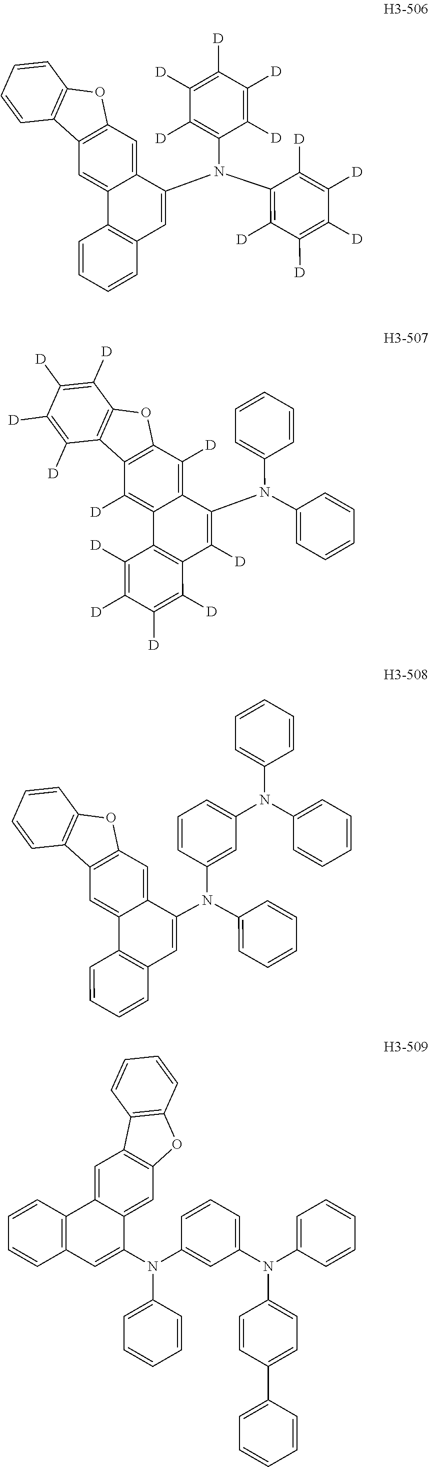 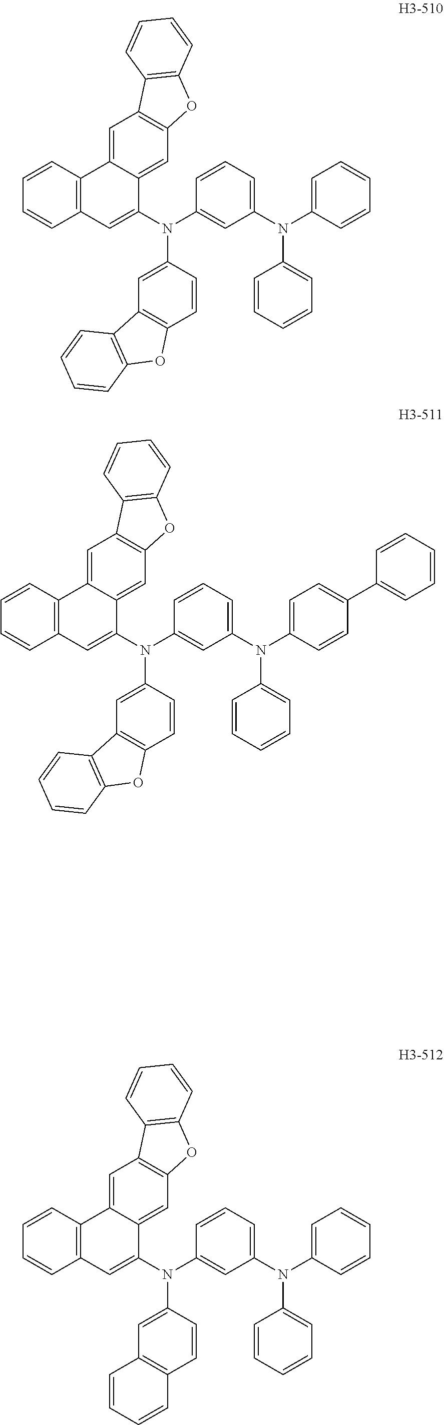 [SMILES: c1ccc(N(c2ccccc2)c2cccc(N(c3ccc4oc5ccccc5c4c3)c3cc4ccccc4c4cc5c(cc34)oc3ccccc35)c2)cc1 ; c1ccc(-c2ccc(N(c3ccccc3)c3cccc(N(c4ccc5oc6ccccc6c5c4)c4cc5ccccc5c5cc6c(cc45)oc4ccccc46)c3)cc2)cc1 ; c1ccc(N(c2ccccc2)c2cccc(N(c3ccc4ccccc4c3)c3cc4ccccc4c4cc5c(cc34)oc3ccccc35)c2)cc1] 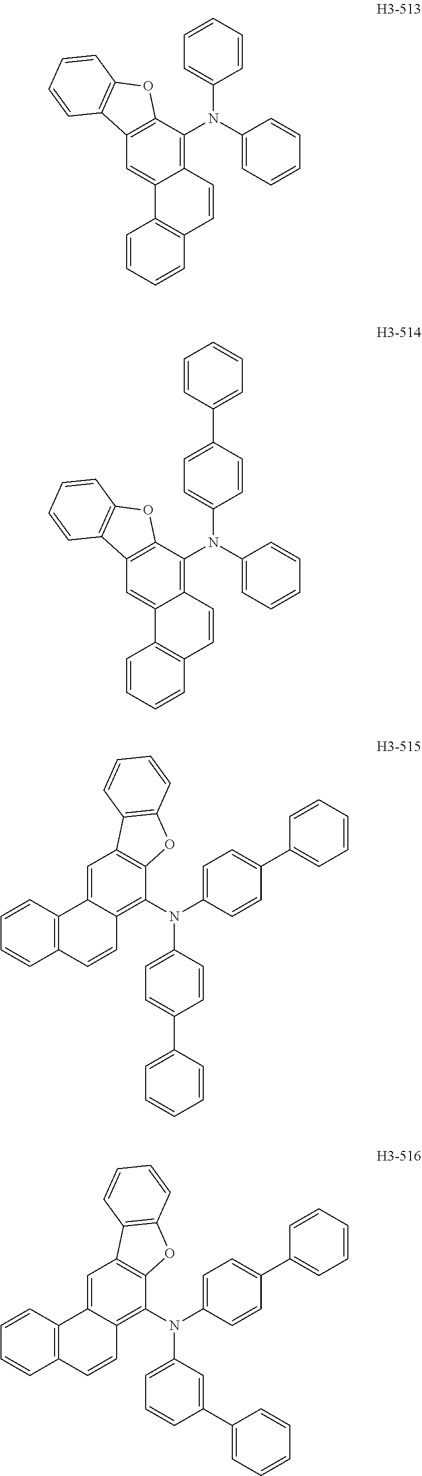 [SMILES: c1ccc(N(c2ccccc2)c2c3ccc4ccccc4c3cc3c2oc2ccccc23)cc1 ; c1ccc(-c2ccc(N(c3ccccc3)c3c4ccc5ccccc5c4cc4c3oc3ccccc34)cc2)cc1 ; c1ccc(-c2ccc(N(c3ccc(-c4ccccc4)cc3)c3c4ccc5ccccc5c4cc4c3oc3ccccc34)cc2)cc1 ; c1ccc(-c2ccc(N(c3cccc(-c4ccccc4)c3)c3c4ccc5ccccc5c4cc4c3oc3ccccc34)cc2)cc1] 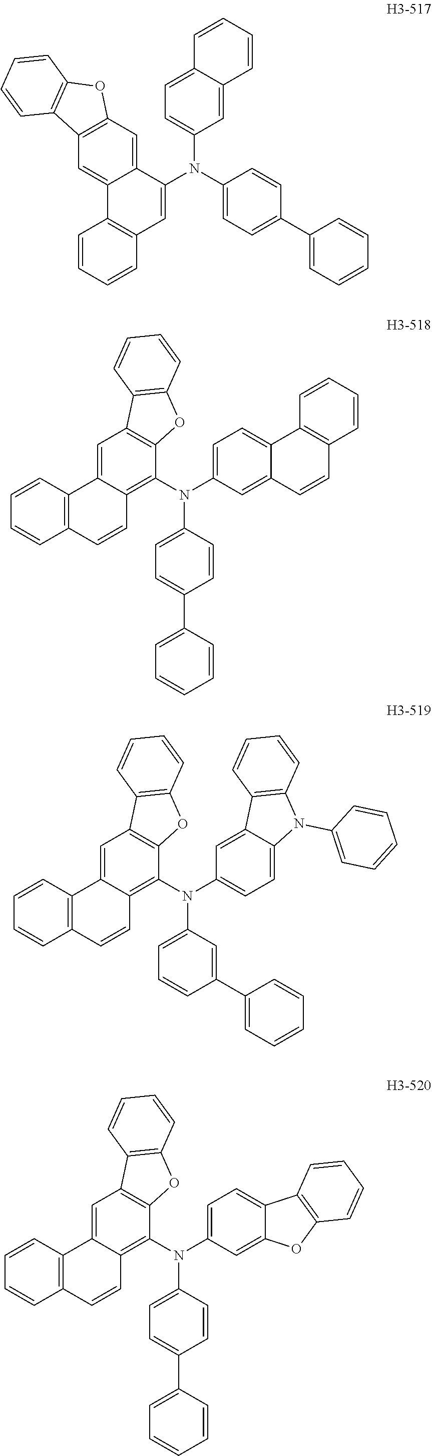 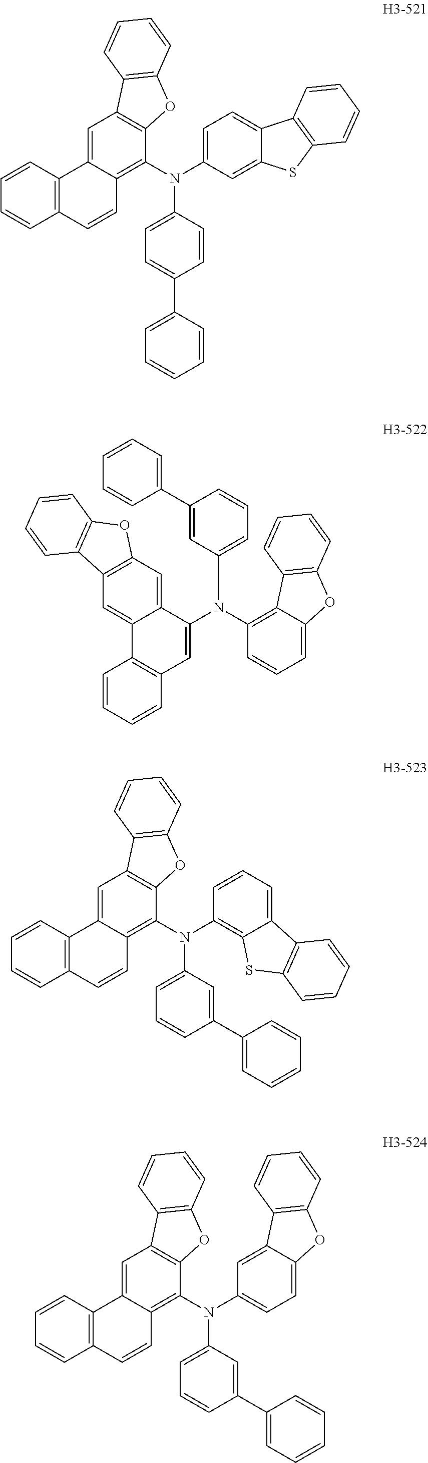 [SMILES: c1ccc(-c2ccc(N(c3ccc4c(c3)sc3ccccc34)c3c4ccc5ccccc5c4cc4c3oc3ccccc34)cc2)cc1 ; c1ccc(-c2cccc(N(c3cc4ccccc4c4cc5c(cc34)oc3ccccc35)c3cccc4oc5ccccc5c34)c2)cc1 ; c1ccc(-c2cccc(N(c3c4ccc5ccccc5c4cc4c3oc3ccccc34)c3cccc4c3sc3ccccc34)c2)cc1 ; c1ccc(-c2cccc(N(c3ccc4oc5ccccc5c4c3)c3c4ccc5ccccc5c4cc4c3oc3ccccc34)c2)cc1] 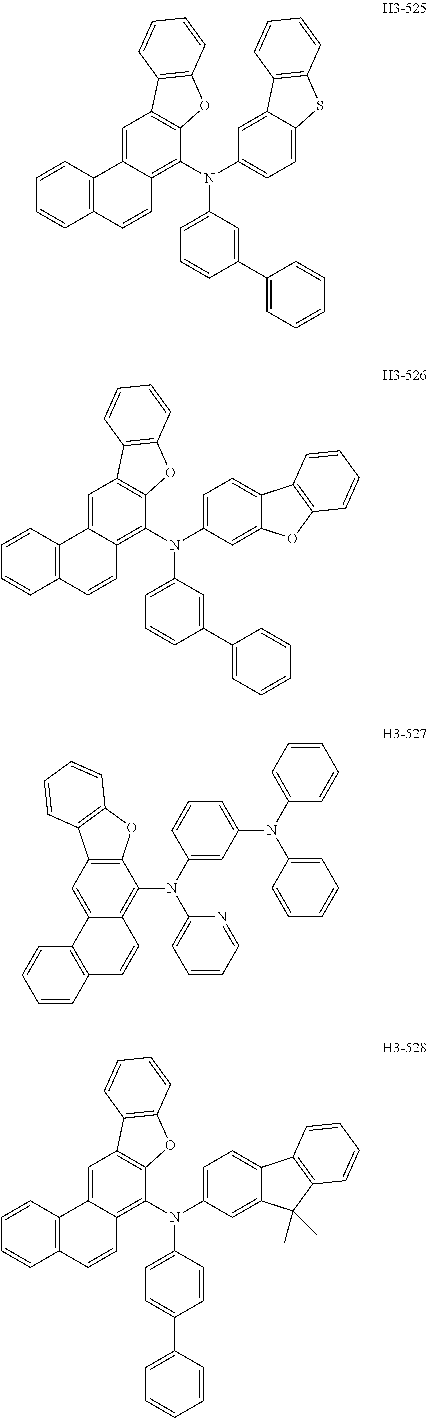 [SMILES: c1ccc(-c2cccc(N(c3ccc4sc5ccccc5c4c3)c3c4ccc5ccccc5c4cc4c3oc3ccccc34)c2)cc1 ; c1ccc(-c2cccc(N(c3ccc4c(c3)oc3ccccc34)c3c4ccc5ccccc5c4cc4c3oc3ccccc34)c2)cc1 ; c1ccc(N(c2ccccc2)c2cccc(N(c3ccccn3)c3c4ccc5ccccc5c4cc4c3oc3ccccc34)c2)cc1 ; CC1(C)c2ccccc2-c2ccc(N(c3ccc(-c4ccccc4)cc3)c3c4ccc5ccccc5c4cc4c3oc3ccccc34)cc21] 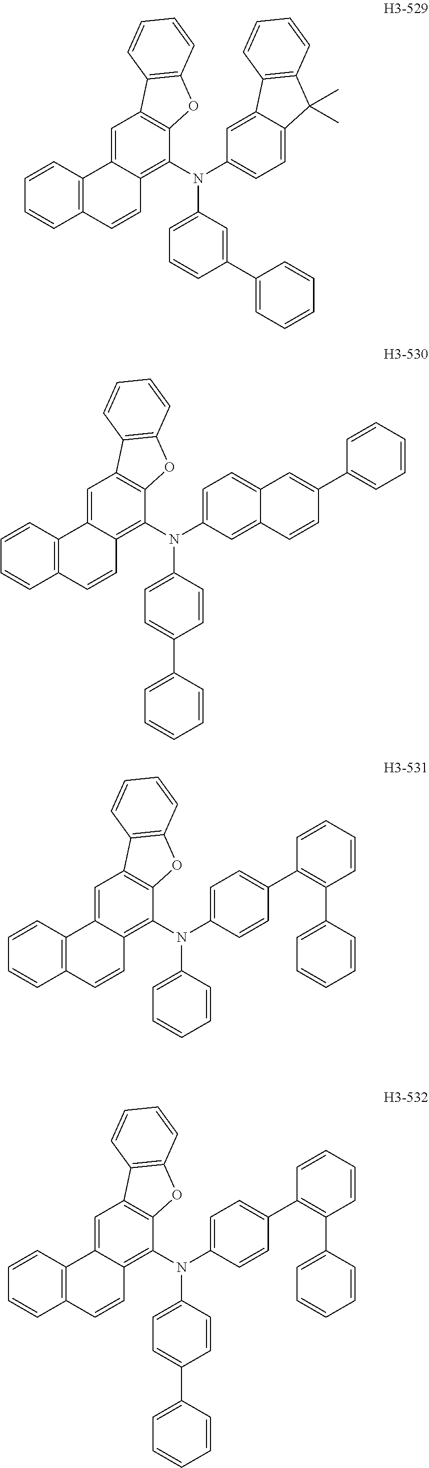 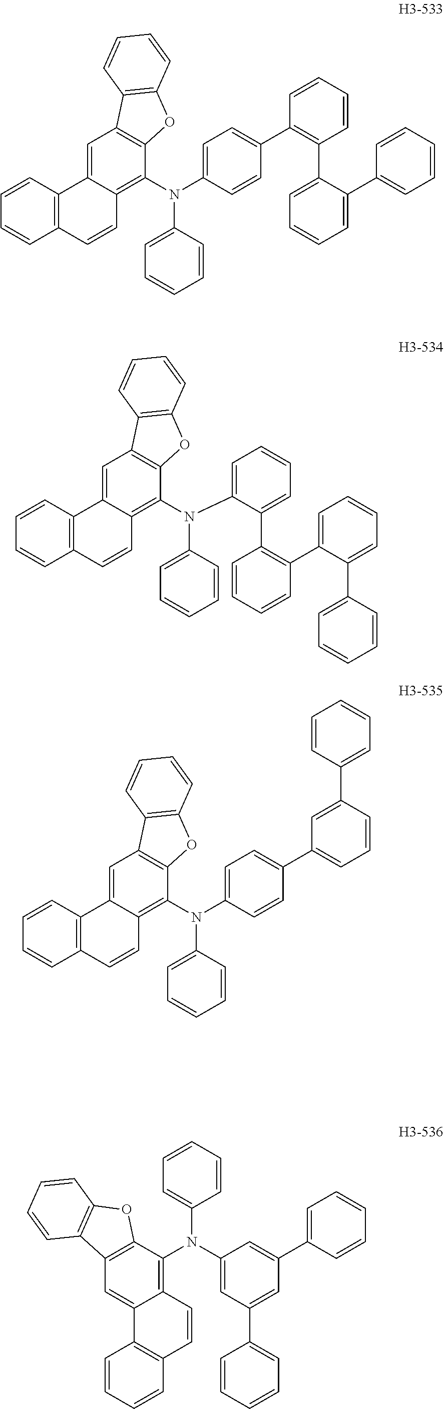 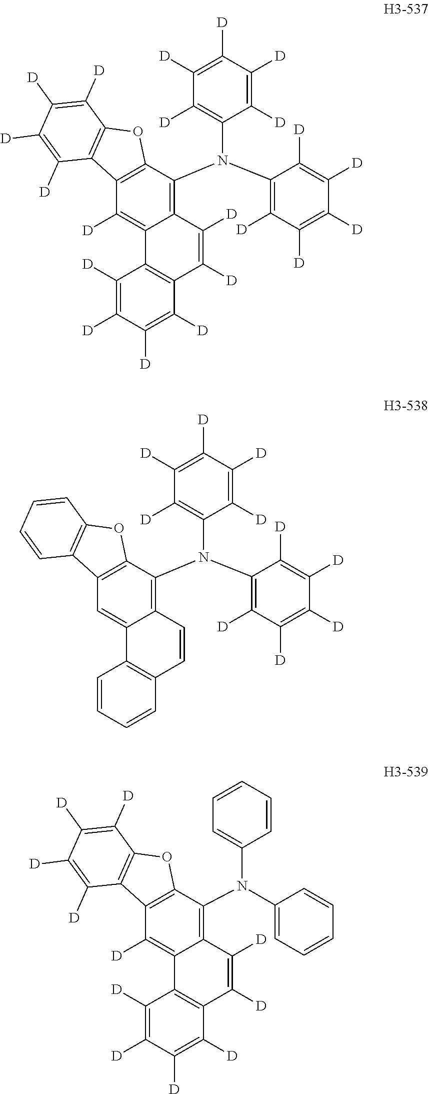 [SMILES: [2H]c1c([2H])c([2H])c(N(c2c([2H])c([2H])c([2H])c([2H])c2[2H])c2c3oc4c([2H])c([2H])c([2H])c([2H])c4c3c([2H])c3c2c([2H])c([2H])c2c([2H])c([2H])c([2H])c([2H])c23)c([2H])c1[2H] ; [2H]c1c([2H])c([2H])c(N(c2c([2H])c([2H])c([2H])c([2H])c2[2H])c2c3ccc4ccccc4c3cc3c2oc2ccccc23)c([2H])c1[2H] ; [2H]c1c([2H])c([2H])c2c(oc3c(N(c4ccccc4)c4ccccc4)c4c([2H])c([2H])c5c([2H])c([2H])c([2H])c([2H])c5c4c([2H])c32)c1[2H]] 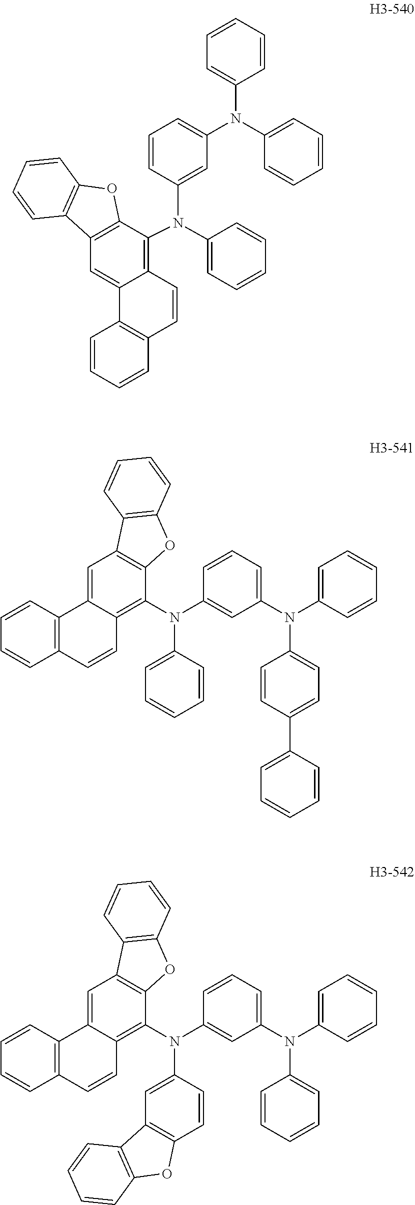 [SMILES: c1ccc(N(c2ccccc2)c2cccc(N(c3ccccc3)c3c4ccc5ccccc5c4cc4c3oc3ccccc34)c2)cc1 ; c1ccc(-c2ccc(N(c3ccccc3)c3cccc(N(c4ccccc4)c4c5ccc6ccccc6c5cc5c4oc4ccccc45)c3)cc2)cc1 ; c1ccc(N(c2ccccc2)c2cccc(N(c3ccc4oc5ccccc5c4c3)c3c4ccc5ccccc5c4cc4c3oc3ccccc34)c2)cc1] 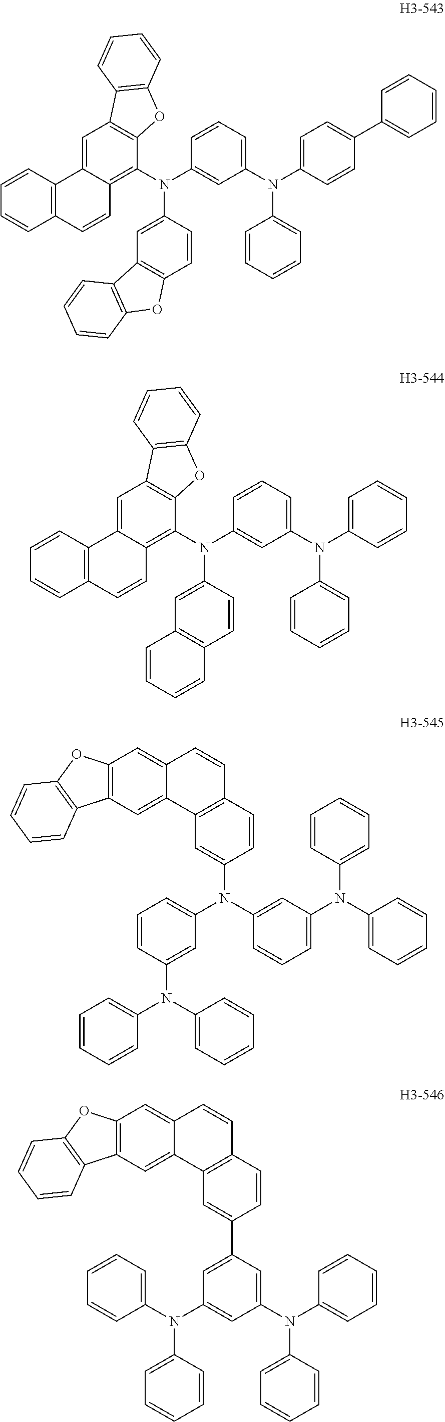 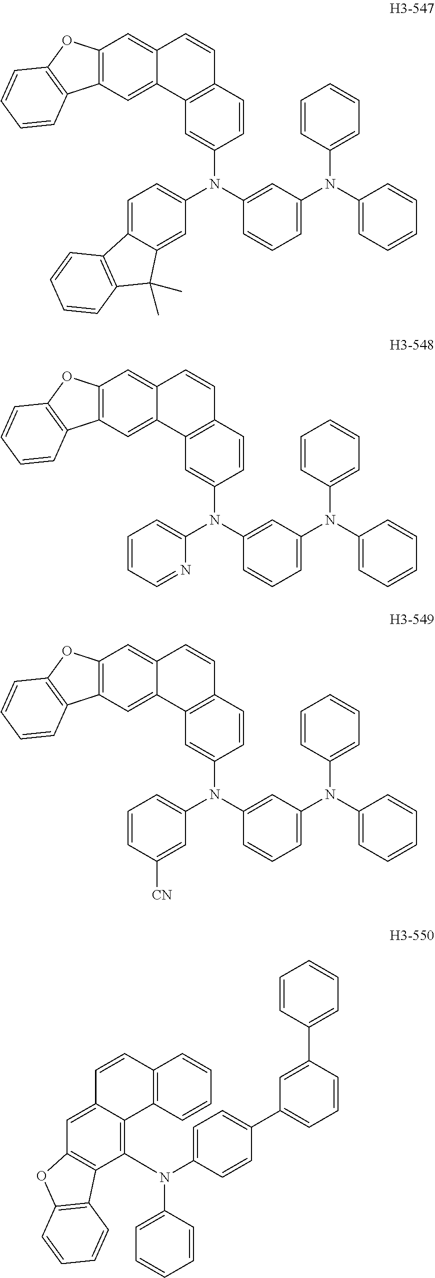 [SMILES: CC1(C)c2ccccc2-c2ccc(N(c3cccc(N(c4ccccc4)c4ccccc4)c3)c3ccc4ccc5cc6oc7ccccc7c6cc5c4c3)cc21 ; c1ccc(N(c2ccccc2)c2cccc(N(c3ccc4ccc5cc6oc7ccccc7c6cc5c4c3)c3ccccn3)c2)cc1 ; N#Cc1cccc(N(c2cccc(N(c3ccccc3)c3ccccc3)c2)c2ccc3ccc4cc5oc6ccccc6c5cc4c3c2)c1 ; c1ccc(-c2cccc(-c3ccc(N(c4ccccc4)c4c5c(ccc6ccccc65)cc5oc6ccccc6c45)cc3)c2)cc1] 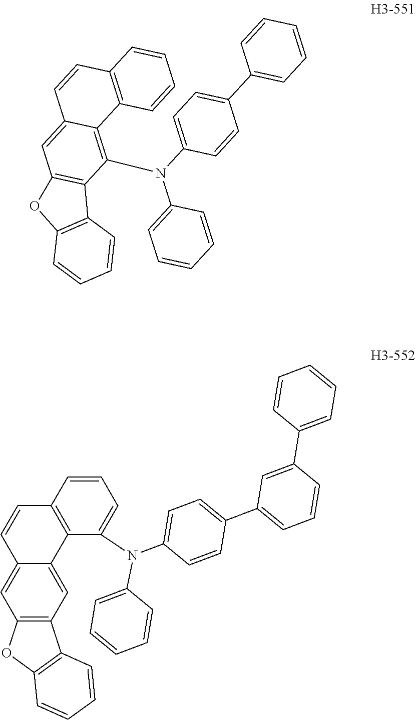 [SMILES: c1ccc(-c2ccc(N(c3ccccc3)c3c4c(ccc5ccccc54)cc4oc5ccccc5c34)cc2)cc1 ; c1ccc(-c2cccc(-c3ccc(N(c4ccccc4)c4cccc5ccc6cc7oc8ccccc8c7cc6c45)cc3)c2)cc1] 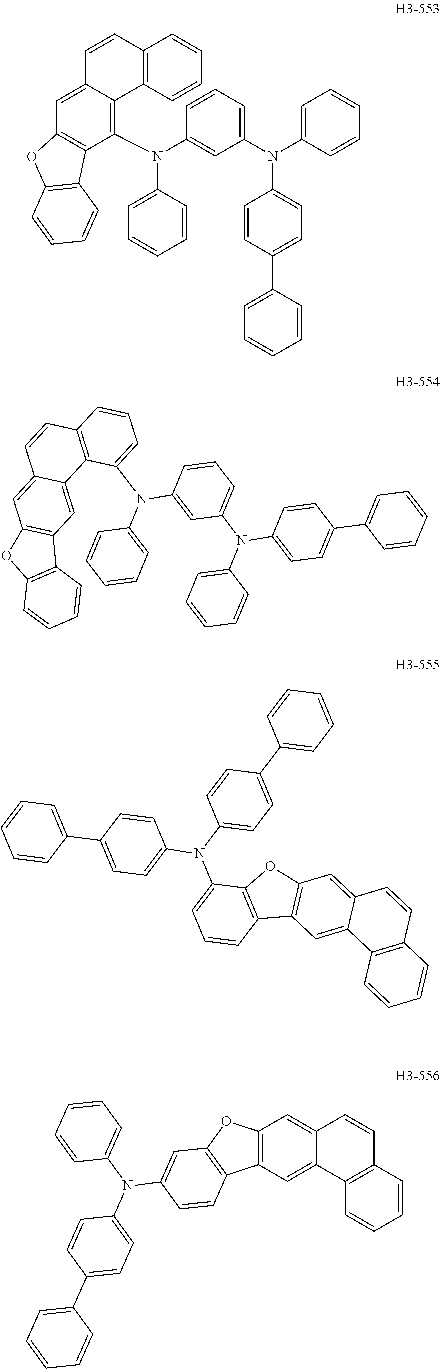 [SMILES: c1ccc(-c2ccc(N(c3ccccc3)c3cccc(N(c4ccccc4)c4c5c(ccc6ccccc65)cc5oc6ccccc6c45)c3)cc2)cc1 ; c1ccc(-c2ccc(N(c3ccccc3)c3cccc(N(c4ccccc4)c4cccc5ccc6cc7oc8ccccc8c7cc6c45)c3)cc2)cc1 ; c1ccc(-c2ccc(N(c3ccc(-c4ccccc4)cc3)c3cccc4c3oc3cc5ccc6ccccc6c5cc34)cc2)cc1 ; c1ccc(-c2ccc(N(c3ccccc3)c3ccc4c(c3)oc3cc5ccc6ccccc6c5cc34)cc2)cc1] 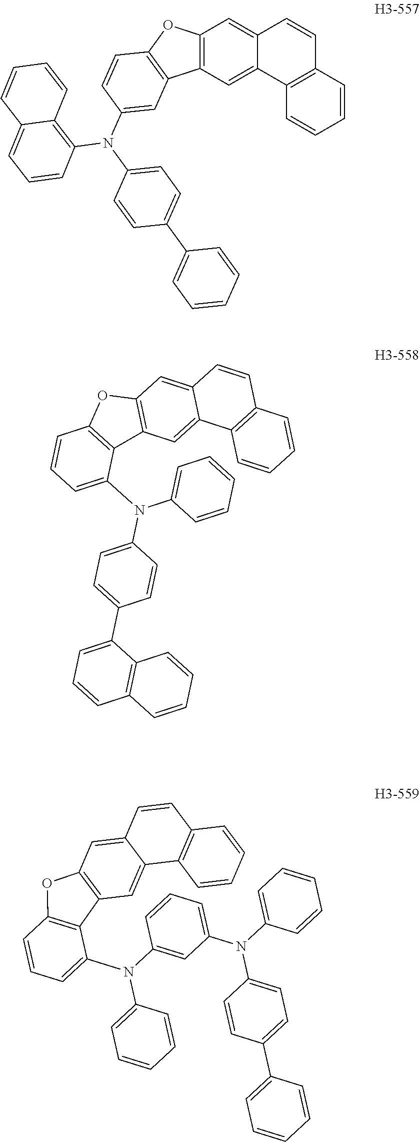 [SMILES: c1ccc(-c2ccc(N(c3ccc4oc5cc6ccc7ccccc7c6cc5c4c3)c3cccc4ccccc34)cc2)cc1 ; c1ccc(N(c2ccc(-c3cccc4ccccc34)cc2)c2cccc3oc4cc5ccc6ccccc6c5cc4c23)cc1 ; c1ccc(-c2ccc(N(c3ccccc3)c3cccc(N(c4ccccc4)c4cccc5oc6cc7ccc8ccccc8c7cc6c45)c3)cc2)cc1] 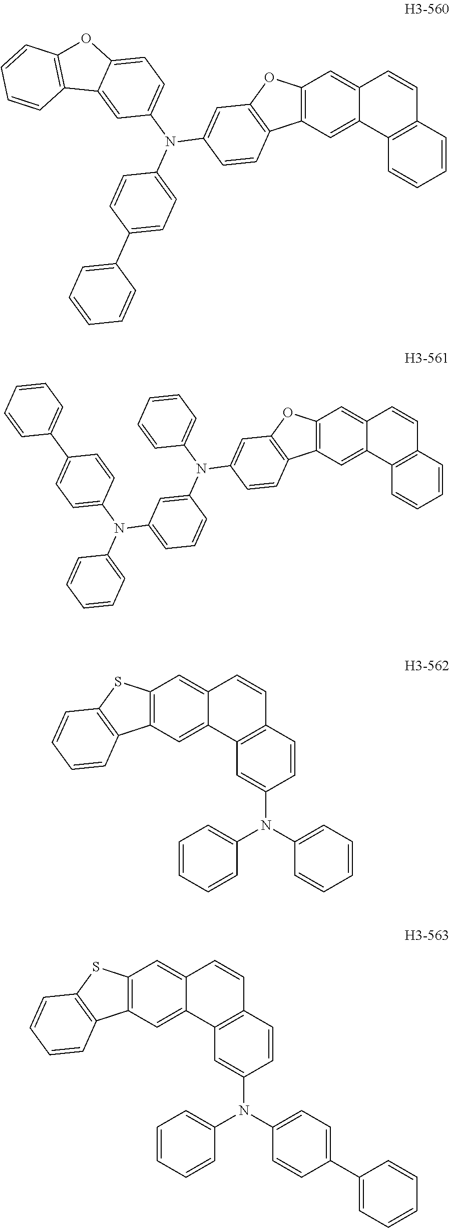 [SMILES: c1ccc(-c2ccc(N(c3ccc4c(c3)oc3cc5ccc6ccccc6c5cc34)c3ccc4oc5ccccc5c4c3)cc2)cc1 ; c1ccc(-c2ccc(N(c3ccccc3)c3cccc(N(c4ccccc4)c4ccc5c(c4)oc4cc6ccc7ccccc7c6cc45)c3)cc2)cc1 ; c1ccc(N(c2ccccc2)c2ccc3ccc4cc5sc6ccccc6c5cc4c3c2)cc1 ; c1ccc(-c2ccc(N(c3ccccc3)c3ccc4ccc5cc6sc7ccccc7c6cc5c4c3)cc2)cc1] 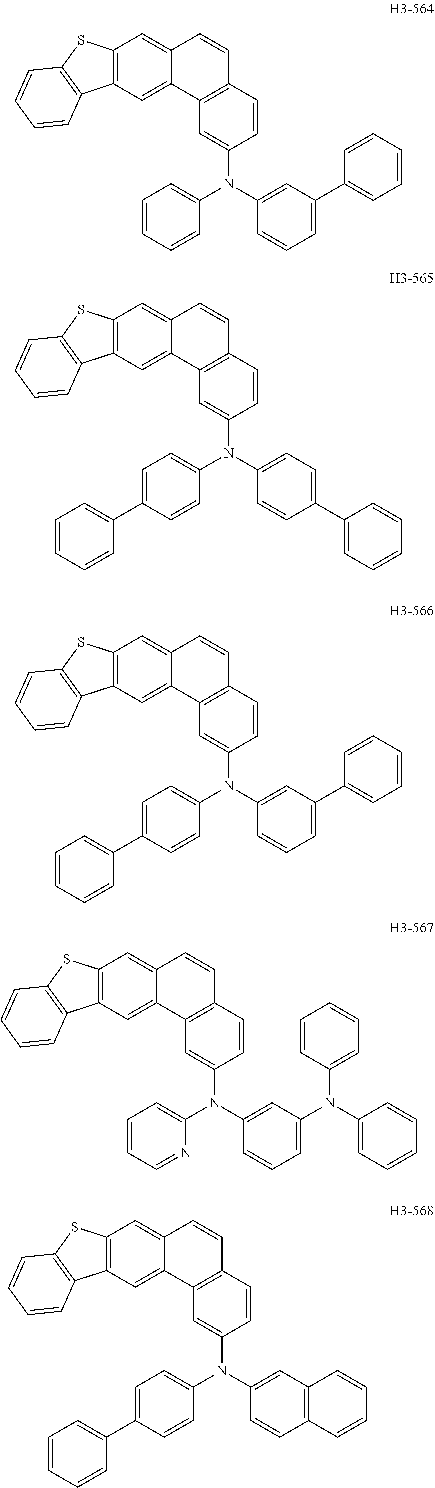 [SMILES: c1ccc(-c2cccc(N(c3ccccc3)c3ccc4ccc5cc6sc7ccccc7c6cc5c4c3)c2)cc1 ; c1ccc(-c2ccc(N(c3ccc(-c4ccccc4)cc3)c3ccc4ccc5cc6sc7ccccc7c6cc5c4c3)cc2)cc1 ; c1ccc(-c2ccc(N(c3cccc(-c4ccccc4)c3)c3ccc4ccc5cc6sc7ccccc7c6cc5c4c3)cc2)cc1 ; c1ccc(N(c2ccccc2)c2cccc(N(c3ccc4ccc5cc6sc7ccccc7c6cc5c4c3)c3ccccn3)c2)cc1 ; c1ccc(-c2ccc(N(c3ccc4ccccc4c3)c3ccc4ccc5cc6sc7ccccc7c6cc5c4c3)cc2)cc1] 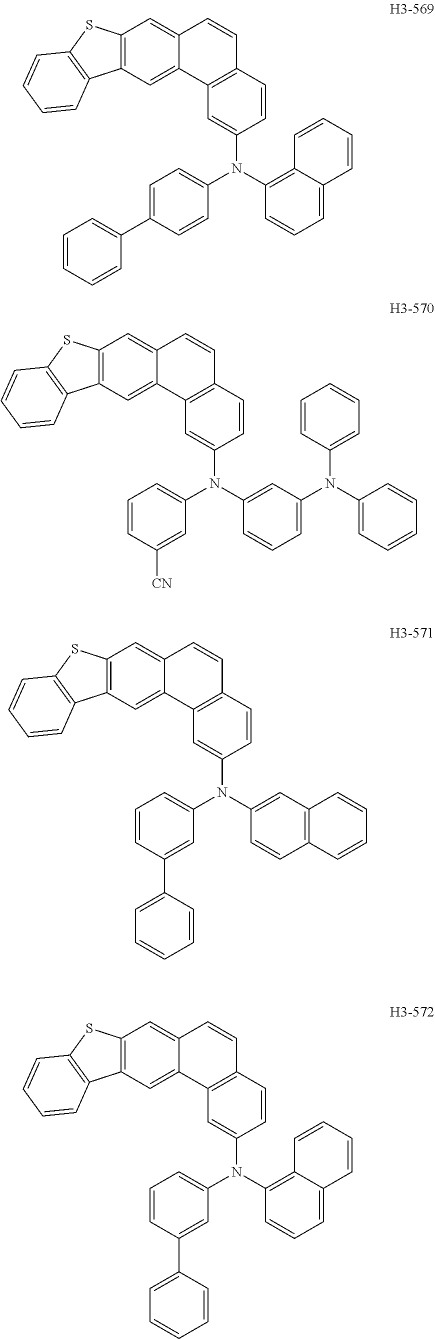 [SMILES: c1ccc(-c2ccc(N(c3ccc4ccc5cc6sc7ccccc7c6cc5c4c3)c3cccc4ccccc34)cc2)cc1 ; N#Cc1cccc(N(c2cccc(N(c3ccccc3)c3ccccc3)c2)c2ccc3ccc4cc5sc6ccccc6c5cc4c3c2)c1 ; c1ccc(-c2cccc(N(c3ccc4ccccc4c3)c3ccc4ccc5cc6sc7ccccc7c6cc5c4c3)c2)cc1 ; c1ccc(-c2cccc(N(c3ccc4ccc5cc6sc7ccccc7c6cc5c4c3)c3cccc4ccccc34)c2)cc1] 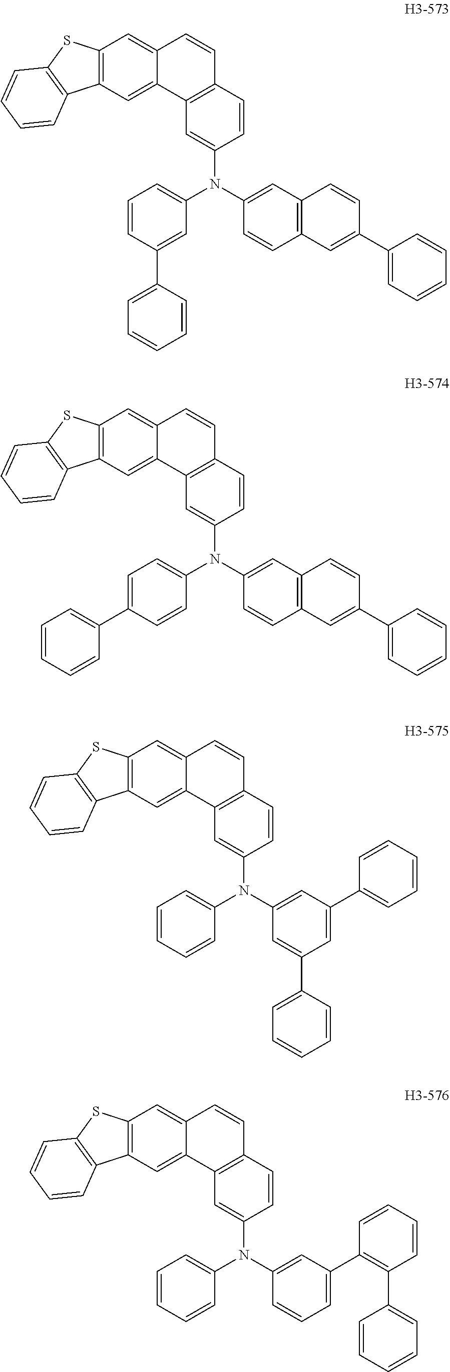 [SMILES: c1ccc(-c2cccc(N(c3ccc4cc(-c5ccccc5)ccc4c3)c3ccc4ccc5cc6sc7ccccc7c6cc5c4c3)c2)cc1 ; c1ccc(-c2ccc(N(c3ccc4cc(-c5ccccc5)ccc4c3)c3ccc4ccc5cc6sc7ccccc7c6cc5c4c3)cc2)cc1 ; c1ccc(-c2cc(-c3ccccc3)cc(N(c3ccccc3)c3ccc4ccc5cc6sc7ccccc7c6cc5c4c3)c2)cc1 ; c1ccc(-c2ccccc2-c2cccc(N(c3ccccc3)c3ccc4ccc5cc6sc7ccccc7c6cc5c4c3)c2)cc1] 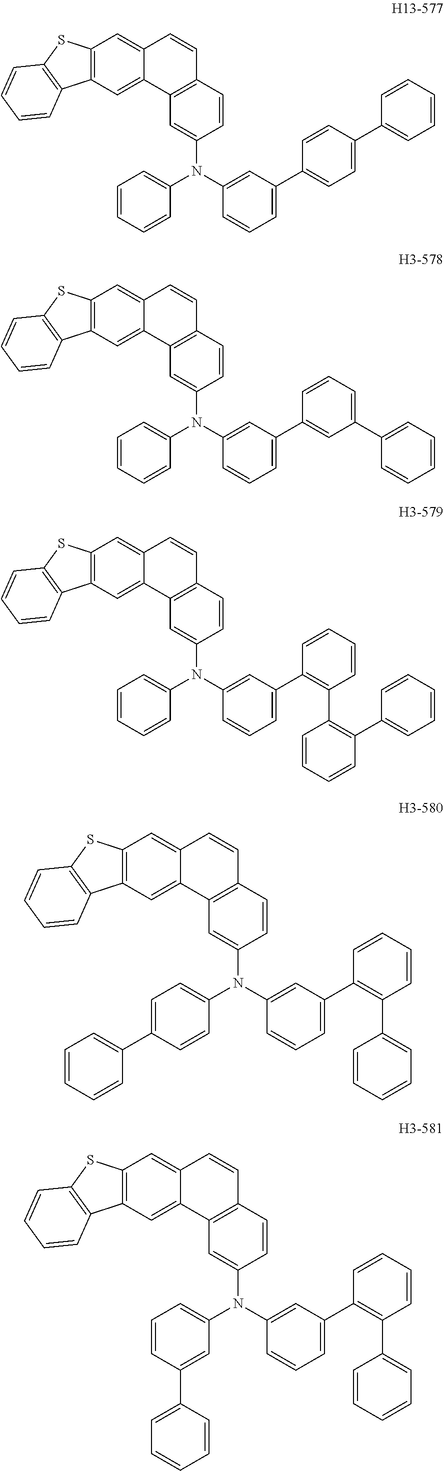 [SMILES: c1ccc(-c2ccc(-c3cccc(N(c4ccccc4)c4ccc5ccc6cc7sc8ccccc8c7cc6c5c4)c3)cc2)cc1 ; c1ccc(-c2cccc(-c3cccc(N(c4ccccc4)c4ccc5ccc6cc7sc8ccccc8c7cc6c5c4)c3)c2)cc1 ; c1ccc(-c2ccccc2-c2ccccc2-c2cccc(N(c3ccccc3)c3ccc4ccc5cc6sc7ccccc7c6cc5c4c3)c2)cc1 ; c1ccc(-c2ccc(N(c3cccc(-c4ccccc4-c4ccccc4)c3)c3ccc4ccc5cc6sc7ccccc7c6cc5c4c3)cc2)cc1 ; c1ccc(-c2cccc(N(c3cccc(-c4ccccc4-c4ccccc4)c3)c3ccc4ccc5cc6sc7ccccc7c6cc5c4c3)c2)cc1] 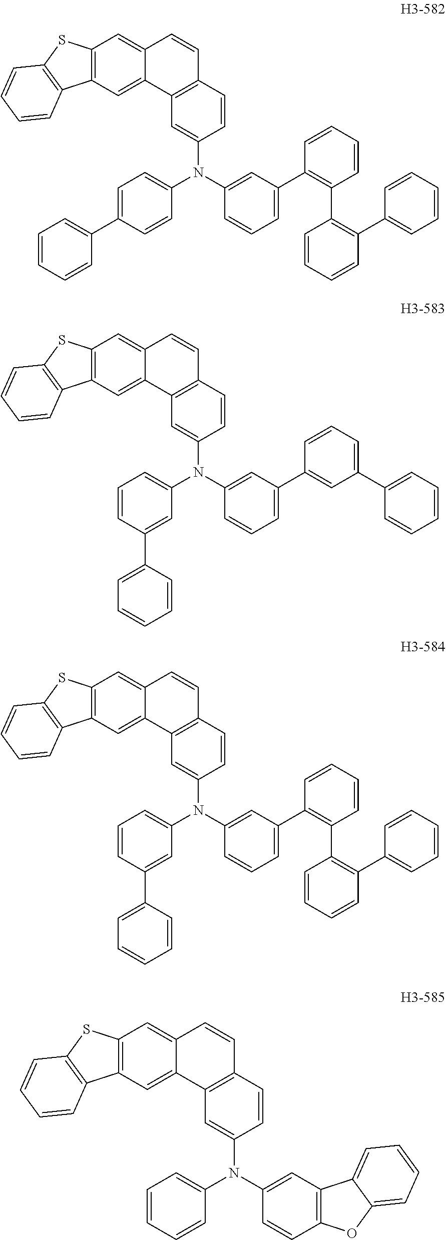 [SMILES: c1ccc(-c2ccc(N(c3cccc(-c4ccccc4-c4ccccc4-c4ccccc4)c3)c3ccc4ccc5cc6sc7ccccc7c6cc5c4c3)cc2)cc1 ; c1ccc(-c2cccc(-c3cccc(N(c4cccc(-c5ccccc5)c4)c4ccc5ccc6cc7sc8ccccc8c7cc6c5c4)c3)c2)cc1 ; c1ccc(-c2cccc(N(c3cccc(-c4ccccc4-c4ccccc4-c4ccccc4)c3)c3ccc4ccc5cc6sc7ccccc7c6cc5c4c3)c2)cc1 ; c1ccc(N(c2ccc3ccc4cc5sc6ccccc6c5cc4c3c2)c2ccc3oc4ccccc4c3c2)cc1] 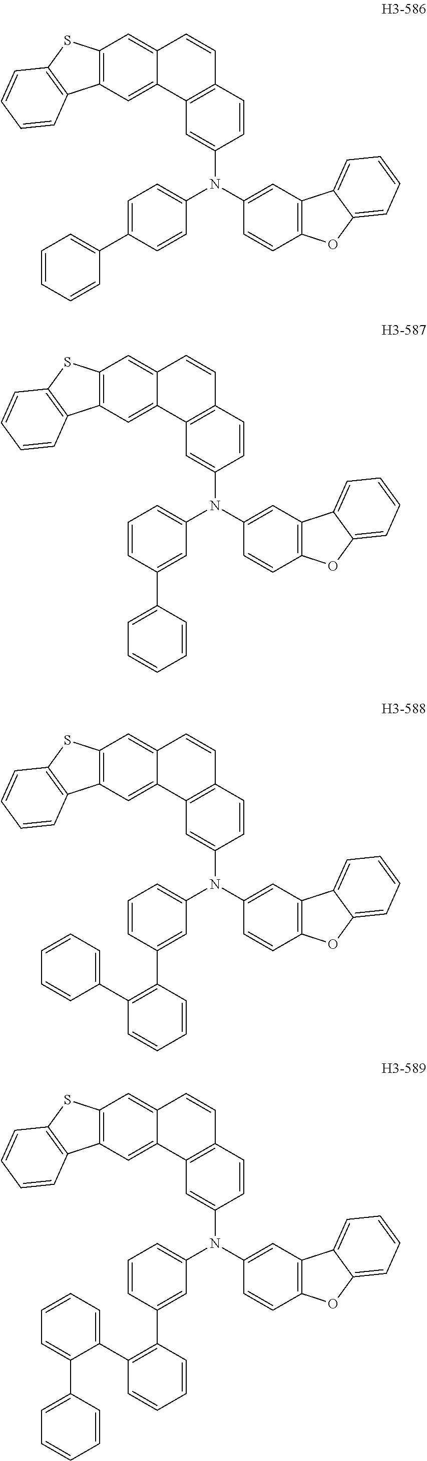 [SMILES: c1ccc(-c2ccc(N(c3ccc4ccc5cc6sc7ccccc7c6cc5c4c3)c3ccc4oc5ccccc5c4c3)cc2)cc1 ; c1ccc(-c2cccc(N(c3ccc4ccc5cc6sc7ccccc7c6cc5c4c3)c3ccc4oc5ccccc5c4c3)c2)cc1 ; c1ccc(-c2ccccc2-c2cccc(N(c3ccc4ccc5cc6sc7ccccc7c6cc5c4c3)c3ccc4oc5ccccc5c4c3)c2)cc1 ; c1ccc(-c2ccccc2-c2ccccc2-c2cccc(N(c3ccc4ccc5cc6sc7ccccc7c6cc5c4c3)c3ccc4oc5ccccc5c4c3)c2)cc1] 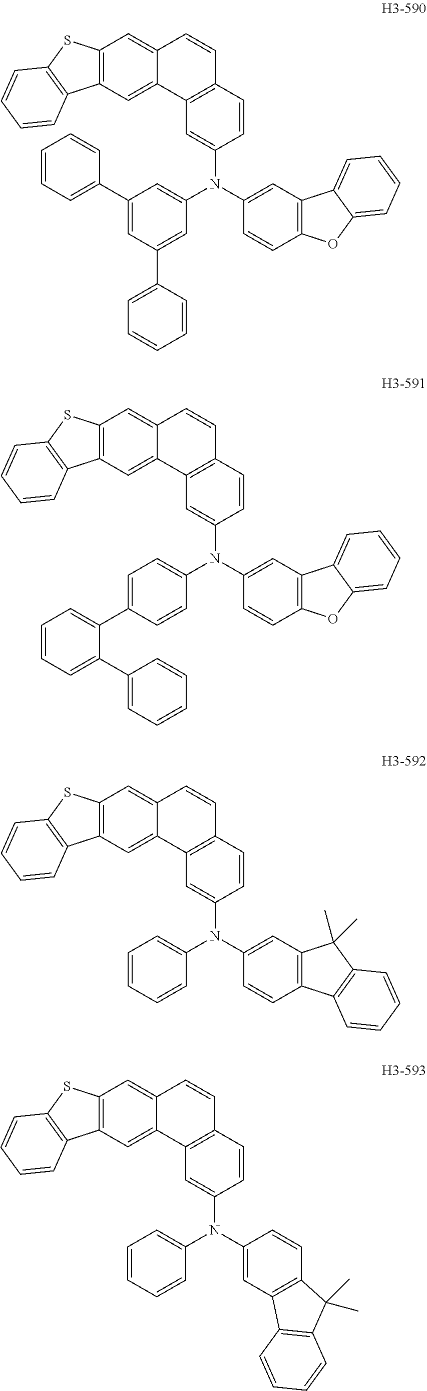 [SMILES: c1ccc(-c2cc(-c3ccccc3)cc(N(c3ccc4ccc5cc6sc7ccccc7c6cc5c4c3)c3ccc4oc5ccccc5c4c3)c2)cc1 ; c1ccc(-c2ccccc2-c2ccc(N(c3ccc4ccc5cc6sc7ccccc7c6cc5c4c3)c3ccc4oc5ccccc5c4c3)cc2)cc1 ; CC1(C)c2ccccc2-c2ccc(N(c3ccccc3)c3ccc4ccc5cc6sc7ccccc7c6cc5c4c3)cc21 ; CC1(C)c2ccccc2-c2cc(N(c3ccccc3)c3ccc4ccc5cc6sc7ccccc7c6cc5c4c3)ccc21] 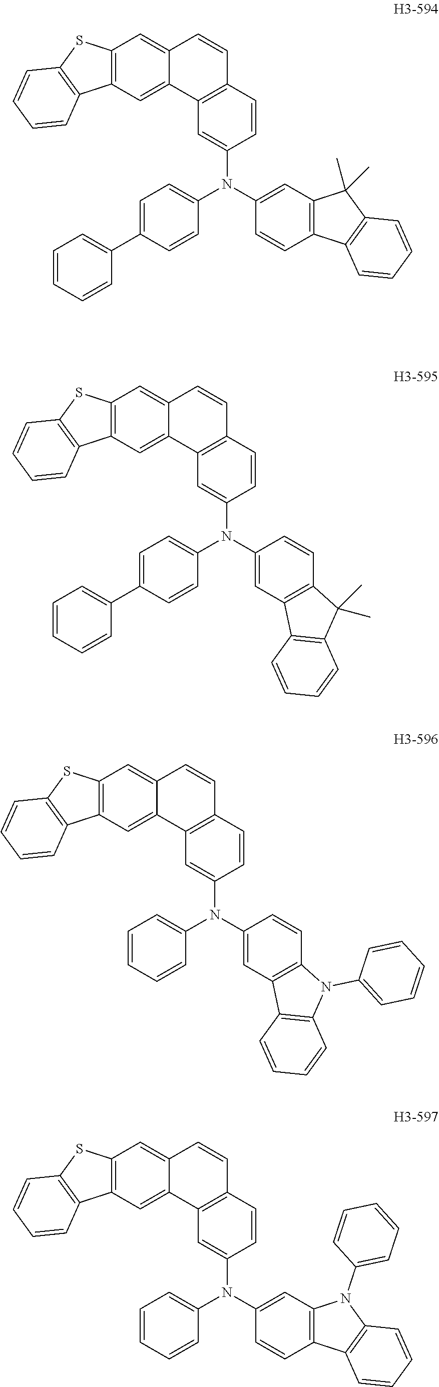 [SMILES: CC1(C)c2ccccc2-c2ccc(N(c3ccc(-c4ccccc4)cc3)c3ccc4ccc5cc6sc7ccccc7c6cc5c4c3)cc21 ; CC1(C)c2ccccc2-c2cc(N(c3ccc(-c4ccccc4)cc3)c3ccc4ccc5cc6sc7ccccc7c6cc5c4c3)ccc21 ; c1ccc(N(c2ccc3ccc4cc5sc6ccccc6c5cc4c3c2)c2ccc3c(c2)c2ccccc2n3-c2ccccc2)cc1 ; c1ccc(N(c2ccc3ccc4cc5sc6ccccc6c5cc4c3c2)c2ccc3c4ccccc4n(-c4ccccc4)c3c2)cc1] 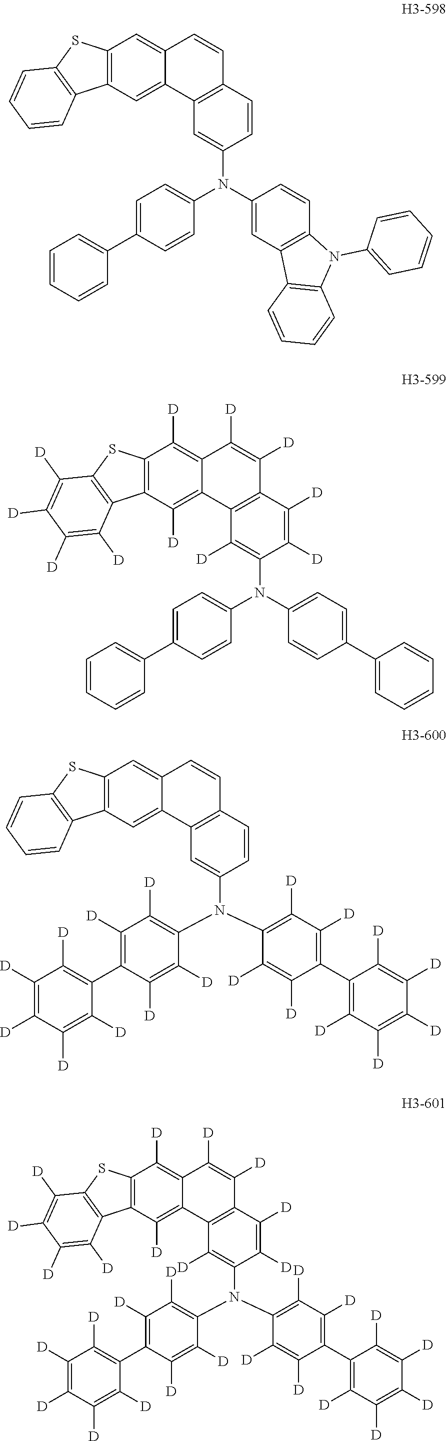 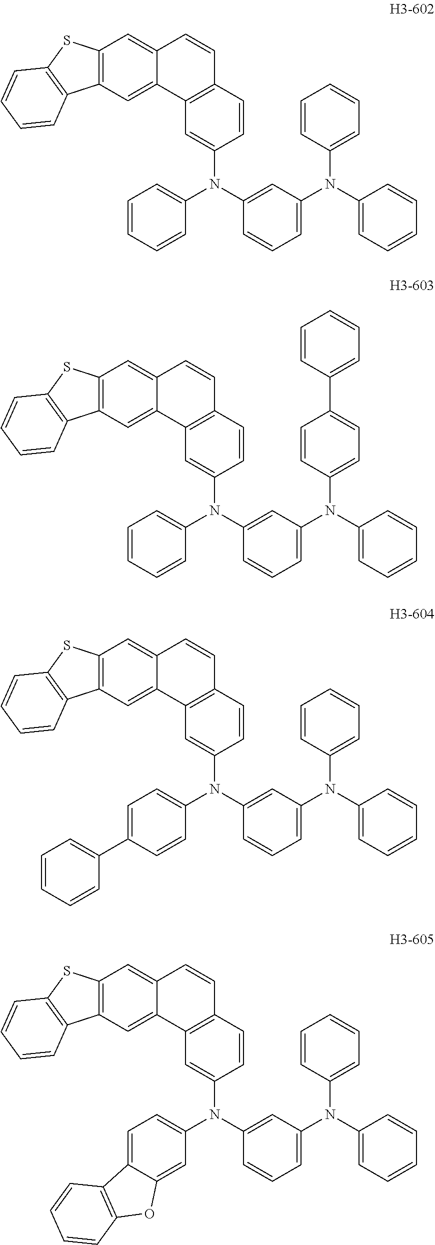 [SMILES: c1ccc(N(c2ccccc2)c2cccc(N(c3ccccc3)c3ccc4ccc5cc6sc7ccccc7c6cc5c4c3)c2)cc1 ; c1ccc(-c2ccc(N(c3ccccc3)c3cccc(N(c4ccccc4)c4ccc5ccc6cc7sc8ccccc8c7cc6c5c4)c3)cc2)cc1 ; c1ccc(-c2ccc(N(c3cccc(N(c4ccccc4)c4ccccc4)c3)c3ccc4ccc5cc6sc7ccccc7c6cc5c4c3)cc2)cc1 ; c1ccc(N(c2ccccc2)c2cccc(N(c3ccc4c(c3)oc3ccccc34)c3ccc4ccc5cc6sc7ccccc7c6cc5c4c3)c2)cc1] 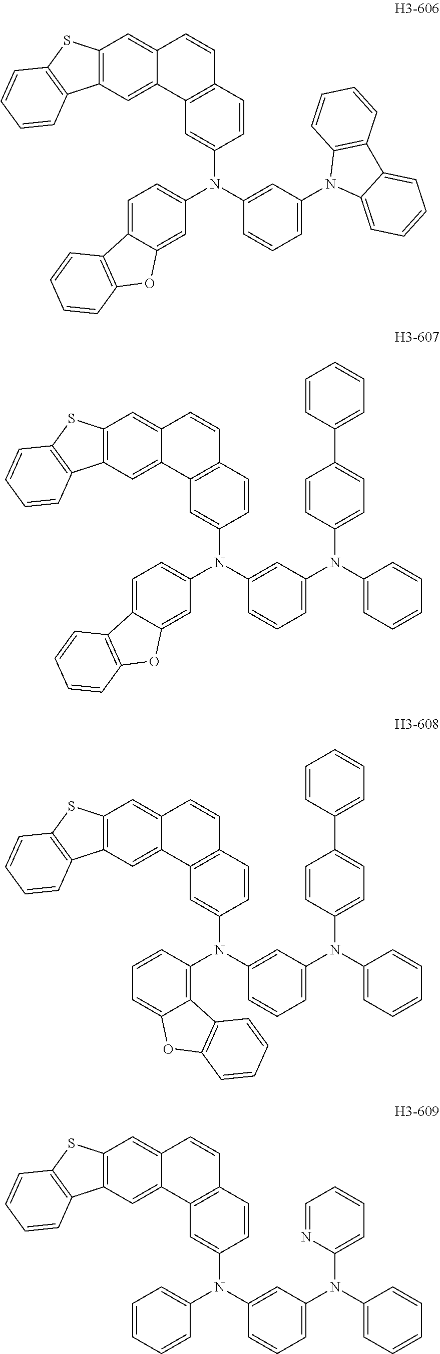 [SMILES: c1cc(N(c2ccc3c(c2)oc2ccccc23)c2ccc3ccc4cc5sc6ccccc6c5cc4c3c2)cc(-n2c3ccccc3c3ccccc32)c1 ; c1ccc(-c2ccc(N(c3ccccc3)c3cccc(N(c4ccc5c(c4)oc4ccccc45)c4ccc5ccc6cc7sc8ccccc8c7cc6c5c4)c3)cc2)cc1 ; c1ccc(-c2ccc(N(c3ccccc3)c3cccc(N(c4ccc5ccc6cc7sc8ccccc8c7cc6c5c4)c4cccc5oc6ccccc6c45)c3)cc2)cc1 ; c1ccc(N(c2cccc(N(c3ccccc3)c3ccccn3)c2)c2ccc3ccc4cc5sc6ccccc6c5cc4c3c2)cc1] 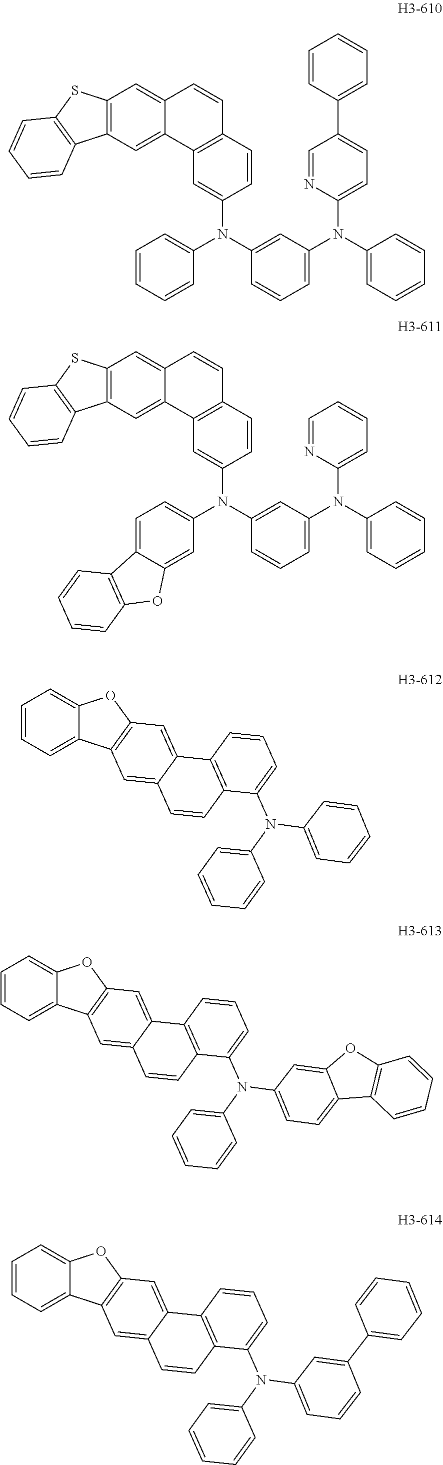 [SMILES: c1ccc(-c2ccc(N(c3ccccc3)c3cccc(N(c4ccccc4)c4ccc5ccc6cc7sc8ccccc8c7cc6c5c4)c3)nc2)cc1 ; c1ccc(N(c2cccc(N(c3ccc4c(c3)oc3ccccc34)c3ccc4ccc5cc6sc7ccccc7c6cc5c4c3)c2)c2ccccn2)cc1 ; c1ccc(N(c2ccccc2)c2cccc3c2ccc2cc4c(cc23)oc2ccccc24)cc1 ; c1ccc(N(c2ccc3c(c2)oc2ccccc23)c2cccc3c2ccc2cc4c(cc23)oc2ccccc24)cc1 ; c1ccc(-c2cccc(N(c3ccccc3)c3cccc4c3ccc3cc5c(cc34)oc3ccccc35)c2)cc1] 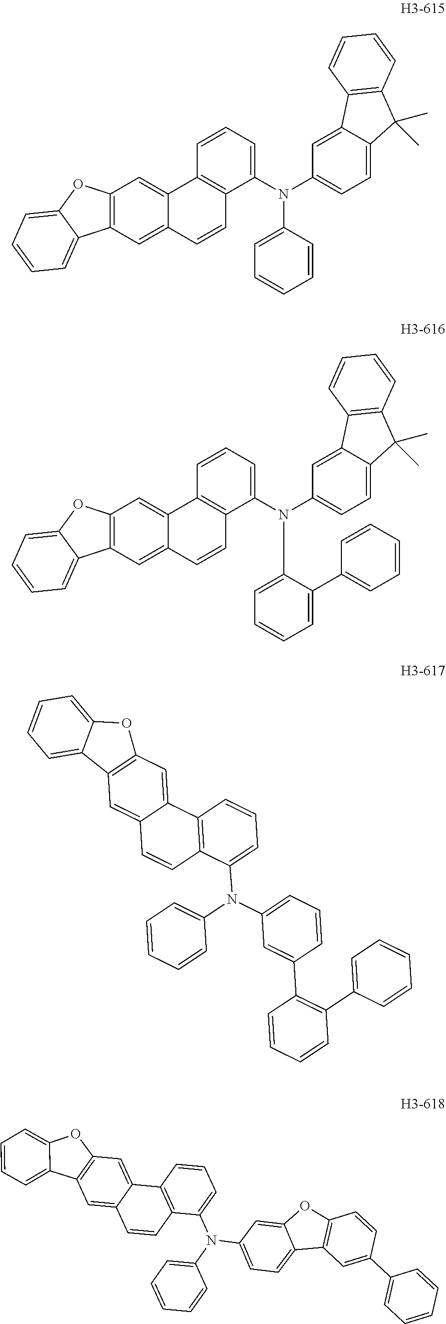 [SMILES: CC1(C)c2ccccc2-c2cc(N(c3ccccc3)c3cccc4c3ccc3cc5c(cc34)oc3ccccc35)ccc21 ; CC1(C)c2ccccc2-c2cc(N(c3ccccc3-c3ccccc3)c3cccc4c3ccc3cc5c(cc34)oc3ccccc35)ccc21 ; c1ccc(-c2ccccc2-c2cccc(N(c3ccccc3)c3cccc4c3ccc3cc5c(cc34)oc3ccccc35)c2)cc1 ; c1ccc(-c2ccc3oc4cc(N(c5ccccc5)c5cccc6c5ccc5cc7c(cc56)oc5ccccc57)ccc4c3c2)cc1] 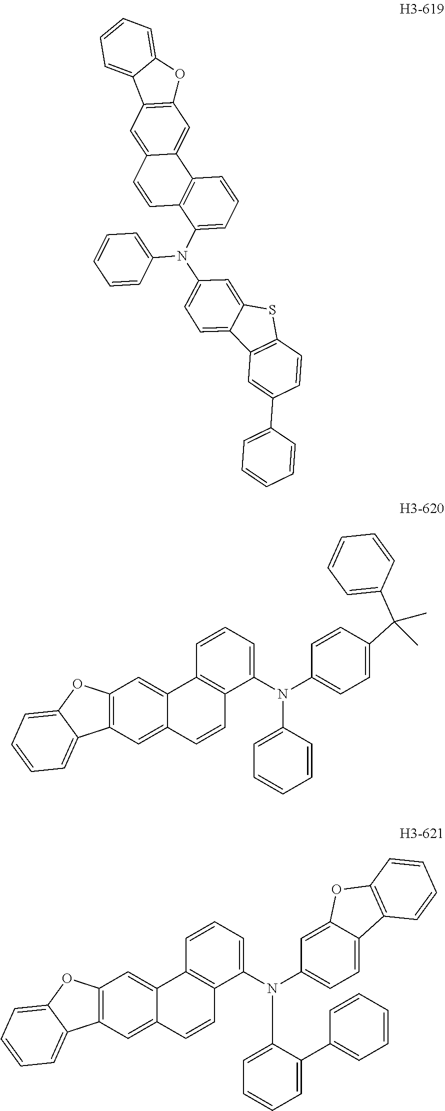 [SMILES: c1ccc(-c2ccc3sc4cc(N(c5ccccc5)c5cccc6c5ccc5cc7c(cc56)oc5ccccc57)ccc4c3c2)cc1 ; CC(C)(c1ccccc1)c1ccc(N(c2ccccc2)c2cccc3c2ccc2cc4c(cc23)oc2ccccc24)cc1 ; c1ccc(-c2ccccc2N(c2ccc3c(c2)oc2ccccc23)c2cccc3c2ccc2cc4c(cc23)oc2ccccc24)cc1] 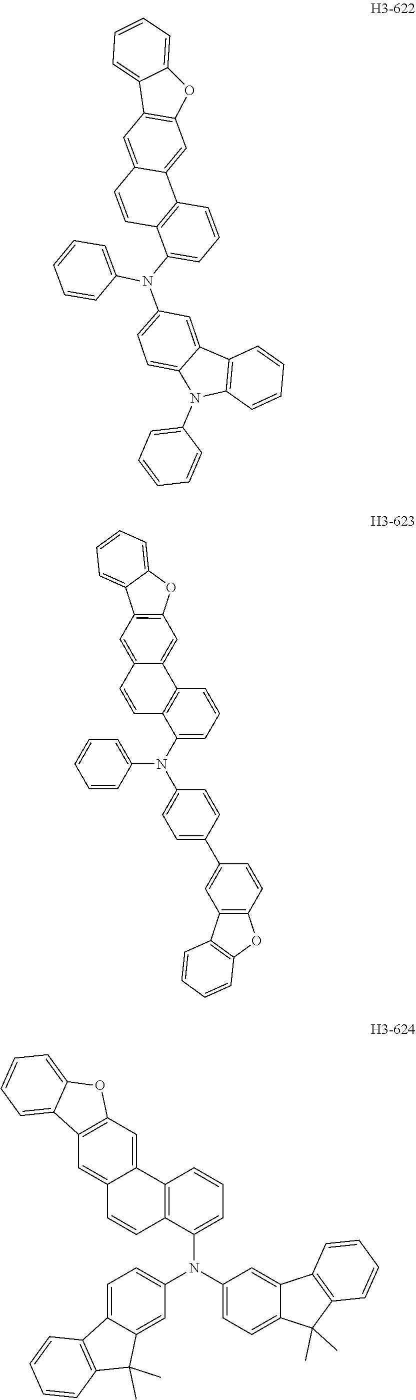 [SMILES: c1ccc(N(c2ccc3c(c2)c2ccccc2n3-c2ccccc2)c2cccc3c2ccc2cc4c(cc23)oc2ccccc24)cc1 ; c1ccc(N(c2ccc(-c3ccc4oc5ccccc5c4c3)cc2)c2cccc3c2ccc2cc4c(cc23)oc2ccccc24)cc1 ; CC1(C)c2ccccc2-c2cc(N(c3ccc4c(c3)C(C)(C)c3ccccc3-4)c3cccc4c3ccc3cc5c(cc34)oc3ccccc35)ccc21] 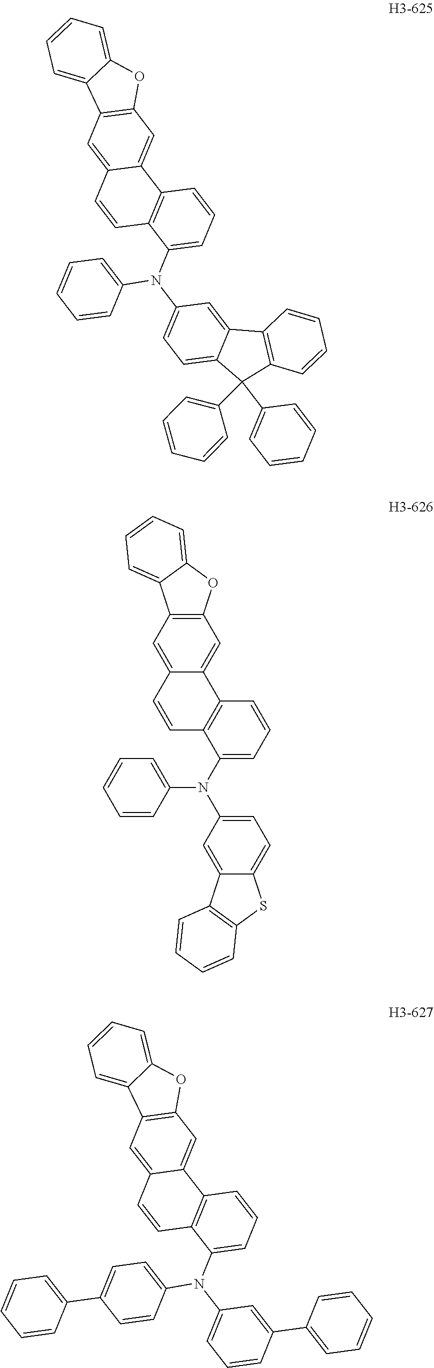 [SMILES: c1ccc(N(c2ccc3c(c2)-c2ccccc2C3(c2ccccc2)c2ccccc2)c2cccc3c2ccc2cc4c(cc23)oc2ccccc24)cc1 ; c1ccc(N(c2ccc3sc4ccccc4c3c2)c2cccc3c2ccc2cc4c(cc23)oc2ccccc24)cc1 ; c1ccc(-c2ccc(N(c3cccc(-c4ccccc4)c3)c3cccc4c3ccc3cc5c(cc34)oc3ccccc35)cc2)cc1] 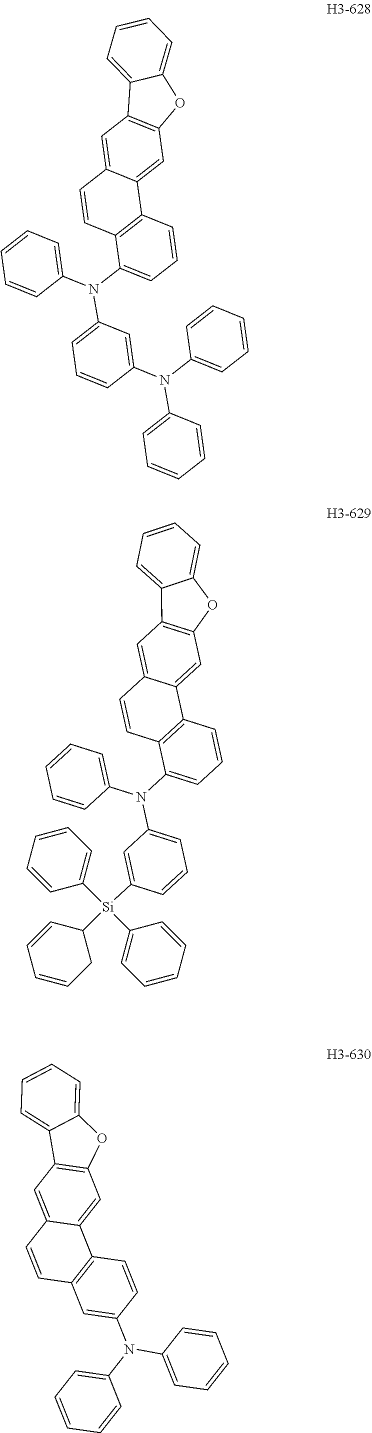 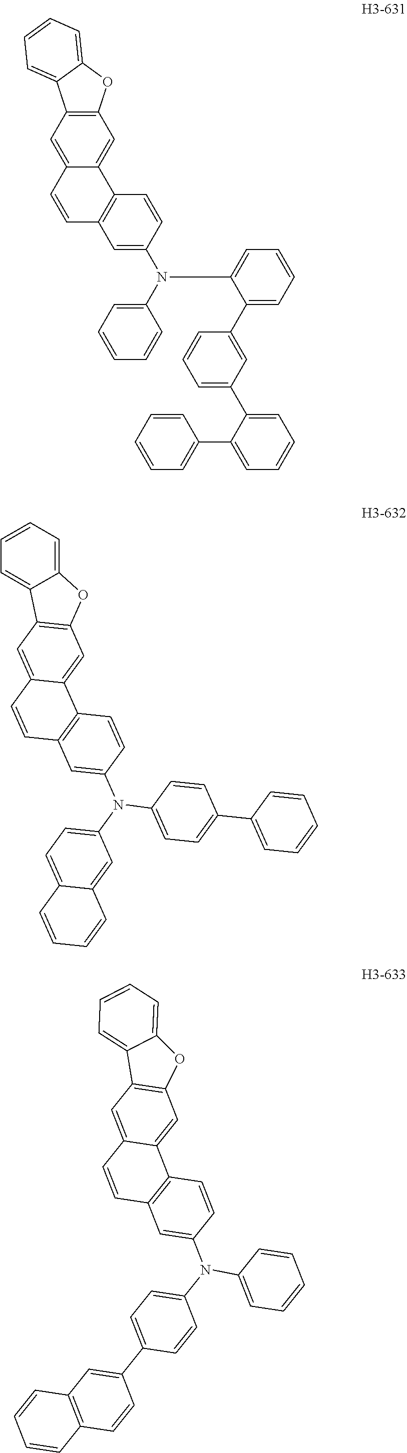 [SMILES: c1ccc(-c2ccccc2-c2cccc(-c3ccccc3N(c3ccccc3)c3ccc4c(ccc5cc6c(cc54)oc4ccccc46)c3)c2)cc1 ; c1ccc(-c2ccc(N(c3ccc4ccccc4c3)c3ccc4c(ccc5cc6c(cc54)oc4ccccc46)c3)cc2)cc1 ; c1ccc(N(c2ccc(-c3ccc4ccccc4c3)cc2)c2ccc3c(ccc4cc5c(cc43)oc3ccccc35)c2)cc1] 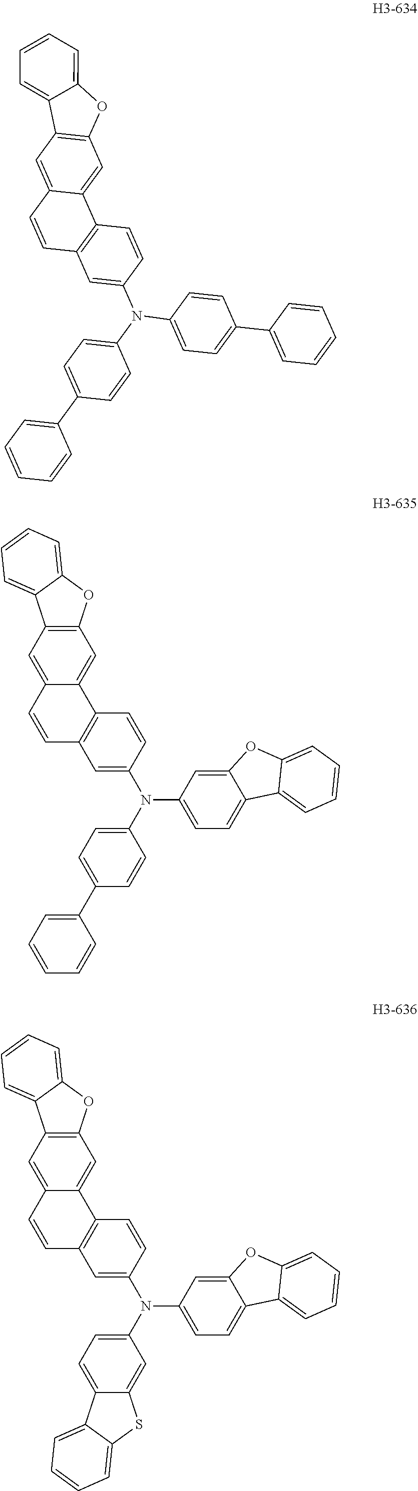 [SMILES: c1ccc(-c2ccc(N(c3ccc(-c4ccccc4)cc3)c3ccc4c(ccc5cc6c(cc54)oc4ccccc46)c3)cc2)cc1 ; c1ccc(-c2ccc(N(c3ccc4c(ccc5cc6c(cc54)oc4ccccc46)c3)c3ccc4c(c3)oc3ccccc34)cc2)cc1 ; c1ccc2c(c1)oc1cc(N(c3ccc4c(ccc5cc6c(cc54)oc4ccccc46)c3)c3ccc4c(c3)sc3ccccc34)ccc12] 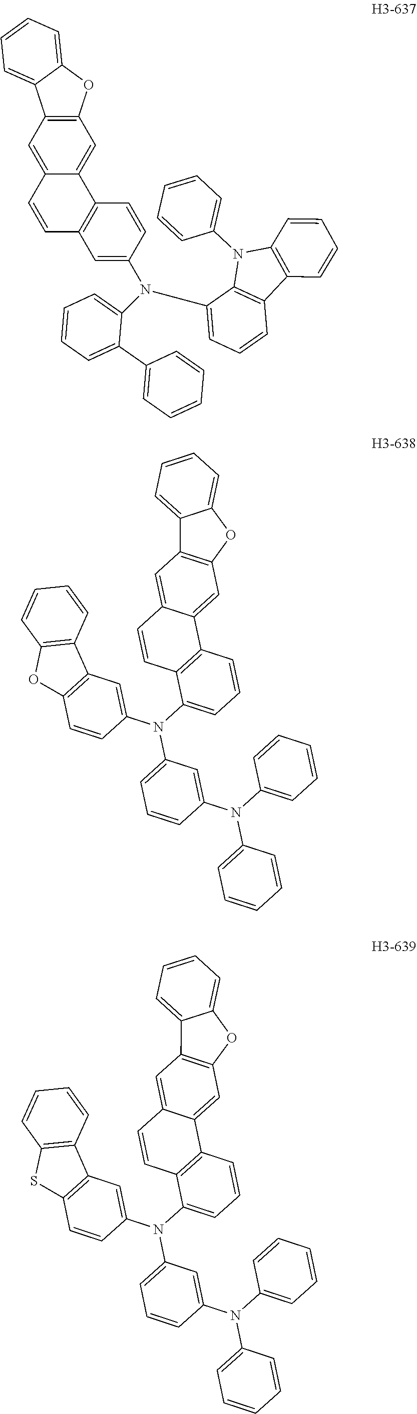 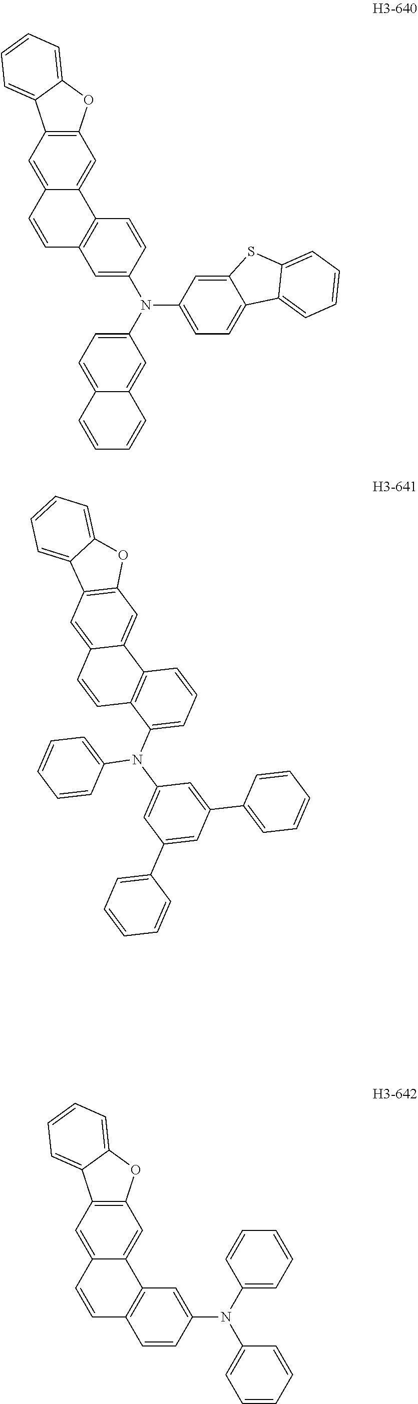 [SMILES: c1ccc2cc(N(c3ccc4c(ccc5cc6c(cc54)oc4ccccc46)c3)c3ccc4c(c3)sc3ccccc34)ccc2c1 ; c1ccc(-c2cc(-c3ccccc3)cc(N(c3ccccc3)c3cccc4c3ccc3cc5c(cc34)oc3ccccc35)c2)cc1 ; c1ccc(N(c2ccccc2)c2ccc3ccc4cc5c(cc4c3c2)oc2ccccc25)cc1] 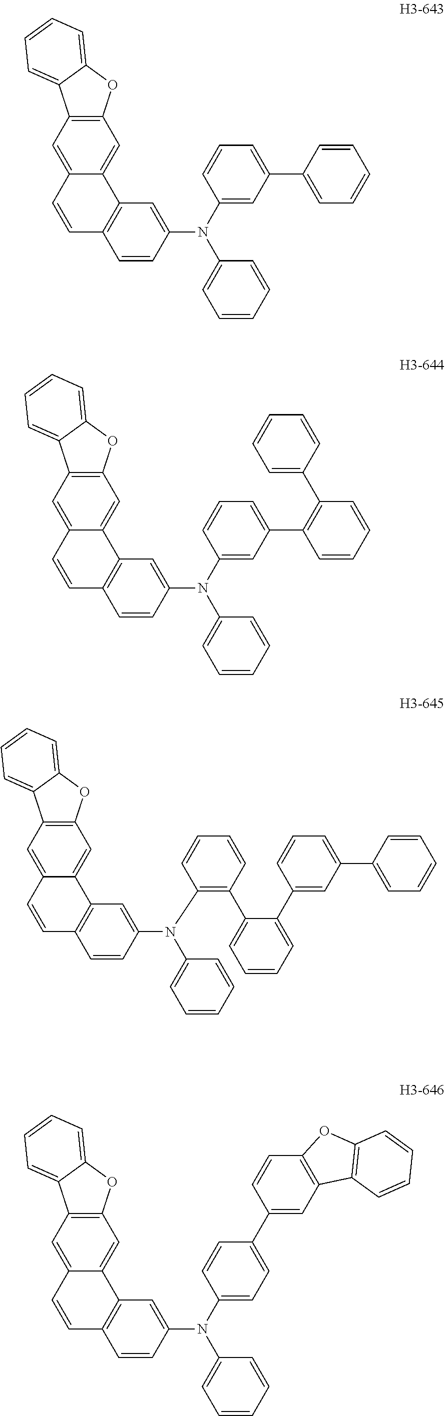 [SMILES: c1ccc(-c2cccc(N(c3ccccc3)c3ccc4ccc5cc6c(cc5c4c3)oc3ccccc36)c2)cc1 ; c1ccc(-c2ccccc2-c2cccc(N(c3ccccc3)c3ccc4ccc5cc6c(cc5c4c3)oc3ccccc36)c2)cc1 ; c1ccc(-c2cccc(-c3ccccc3-c3ccccc3N(c3ccccc3)c3ccc4ccc5cc6c(cc5c4c3)oc3ccccc36)c2)cc1 ; c1ccc(N(c2ccc(-c3ccc4oc5ccccc5c4c3)cc2)c2ccc3ccc4cc5c(cc4c3c2)oc2ccccc25)cc1] 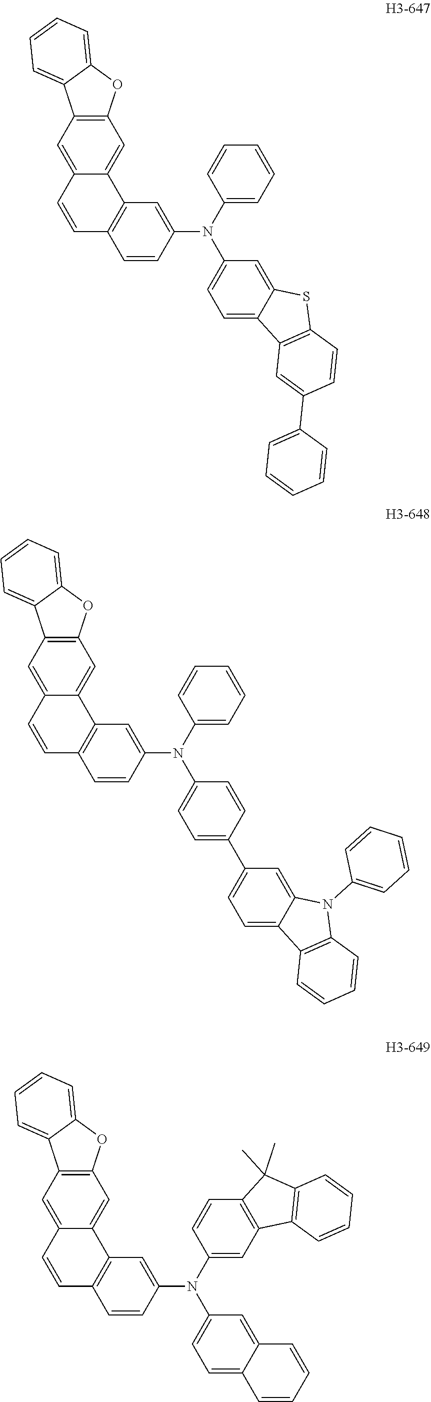 [SMILES: c1ccc(-c2ccc3sc4cc(N(c5ccccc5)c5ccc6ccc7cc8c(cc7c6c5)oc5ccccc58)ccc4c3c2)cc1 ; c1ccc(N(c2ccc(-c3ccc4c5ccccc5n(-c5ccccc5)c4c3)cc2)c2ccc3ccc4cc5c(cc4c3c2)oc2ccccc25)cc1 ; CC1(C)c2ccccc2-c2cc(N(c3ccc4ccccc4c3)c3ccc4ccc5cc6c(cc5c4c3)oc3ccccc36)ccc21] 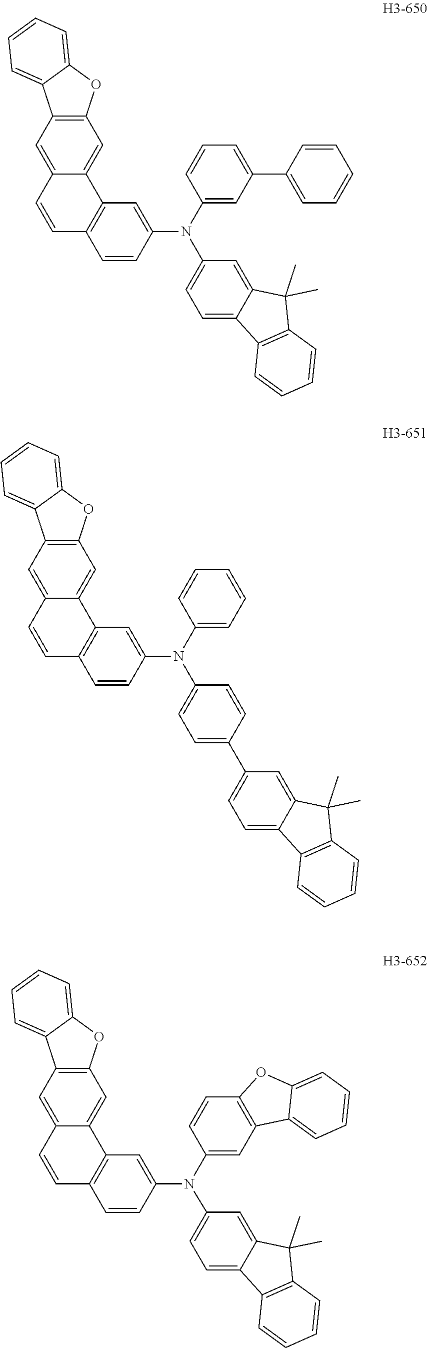 [SMILES: CC1(C)c2ccccc2-c2ccc(N(c3cccc(-c4ccccc4)c3)c3ccc4ccc5cc6c(cc5c4c3)oc3ccccc36)cc21 ; CC1(C)c2ccccc2-c2ccc(-c3ccc(N(c4ccccc4)c4ccc5ccc6cc7c(cc6c5c4)oc4ccccc47)cc3)cc21 ; CC1(C)c2ccccc2-c2ccc(N(c3ccc4ccc5cc6c(cc5c4c3)oc3ccccc36)c3ccc4oc5ccccc5c4c3)cc21] 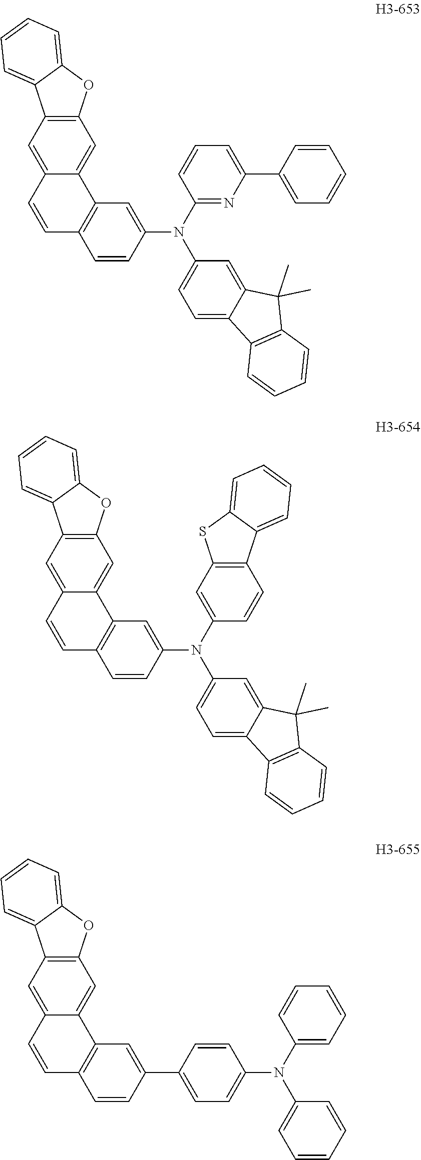 [SMILES: CC1(C)c2ccccc2-c2ccc(N(c3ccc4ccc5cc6c(cc5c4c3)oc3ccccc36)c3cccc(-c4ccccc4)n3)cc21 ; CC1(C)c2ccccc2-c2ccc(N(c3ccc4c(c3)sc3ccccc34)c3ccc4ccc5cc6c(cc5c4c3)oc3ccccc36)cc21 ; c1ccc(N(c2ccccc2)c2ccc(-c3ccc4ccc5cc6c(cc5c4c3)oc3ccccc36)cc2)cc1] 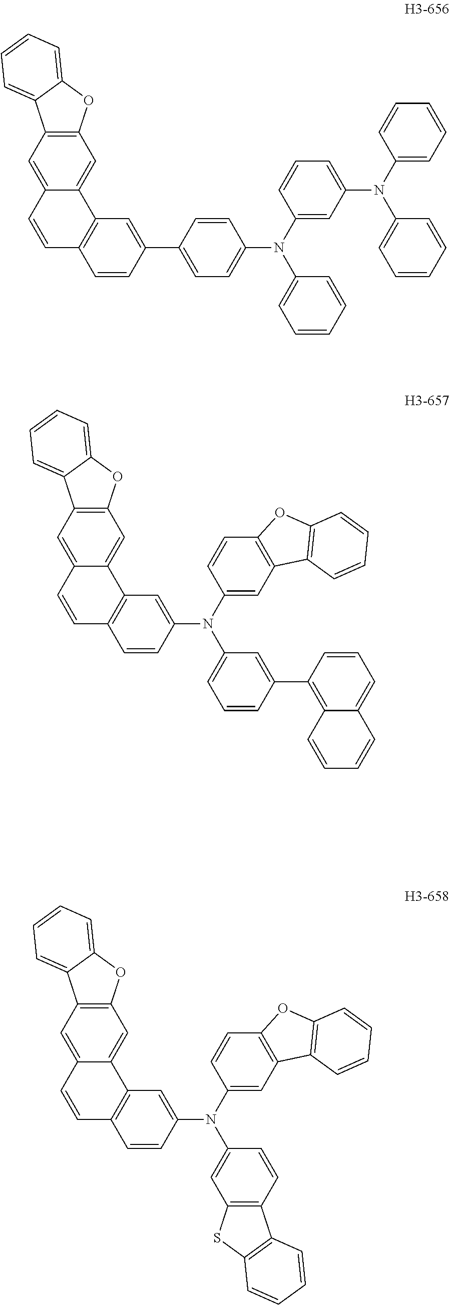 [SMILES: c1ccc(N(c2ccccc2)c2cccc(N(c3ccccc3)c3ccc(-c4ccc5ccc6cc7c(cc6c5c4)oc4ccccc47)cc3)c2)cc1 ; c1cc(-c2cccc3ccccc23)cc(N(c2ccc3ccc4cc5c(cc4c3c2)oc2ccccc25)c2ccc3oc4ccccc4c3c2)c1 ; c1ccc2c(c1)oc1ccc(N(c3ccc4c(c3)sc3ccccc34)c3ccc4ccc5cc6c(cc5c4c3)oc3ccccc36)cc12] 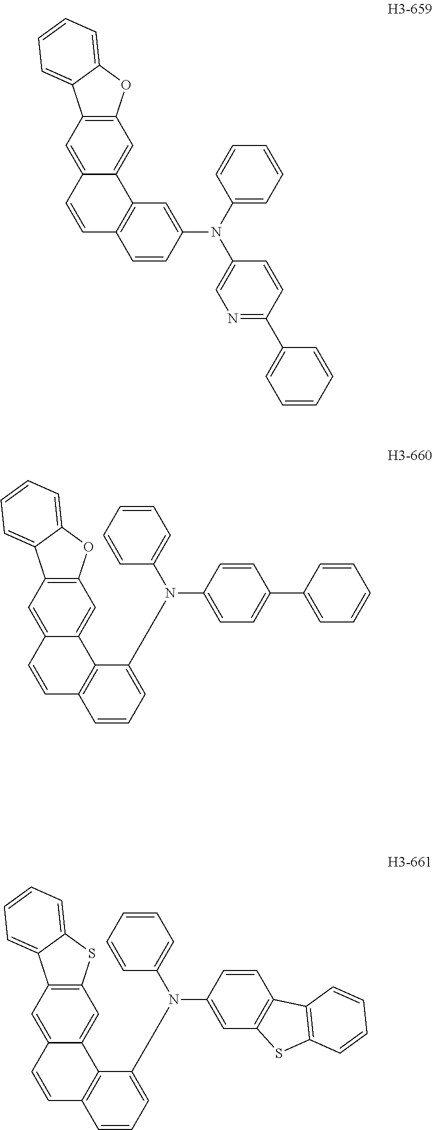 [SMILES: c1ccc(-c2ccc(N(c3ccccc3)c3ccc4ccc5cc6c(cc5c4c3)oc3ccccc36)cn2)cc1 ; c1ccc(-c2ccc(N(c3ccccc3)c3cccc4ccc5cc6c(cc5c34)oc3ccccc36)cc2)cc1 ; c1ccc(N(c2ccc3c(c2)sc2ccccc23)c2cccc3ccc4cc5c(cc4c23)sc2ccccc25)cc1] 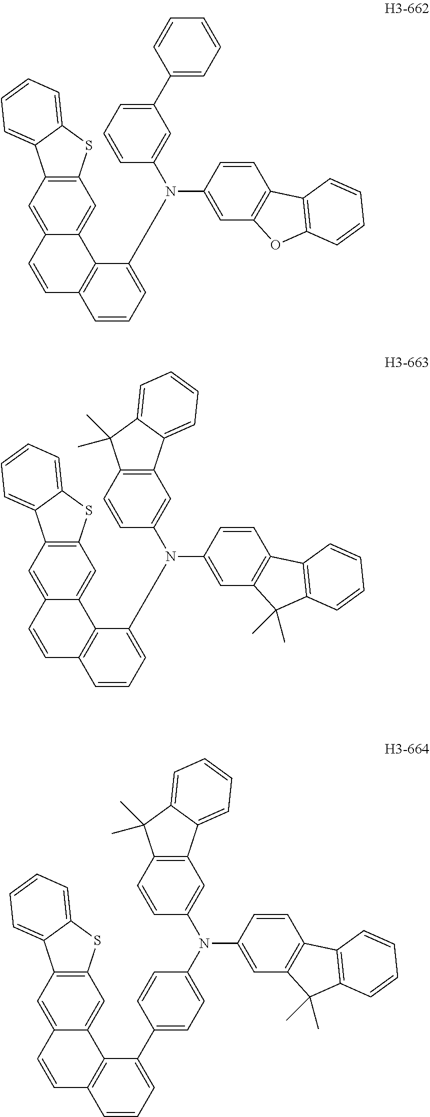 [SMILES: c1ccc(-c2cccc(N(c3ccc4c(c3)oc3ccccc34)c3cccc4ccc5cc6c(cc5c34)sc3ccccc36)c2)cc1 ; CC1(C)c2ccccc2-c2cc(N(c3ccc4c(c3)C(C)(C)c3ccccc3-4)c3cccc4ccc5cc6c(cc5c34)sc3ccccc36)ccc21 ; CC1(C)c2ccccc2-c2cc(N(c3ccc(-c4cccc5ccc6cc7c(cc6c45)sc4ccccc47)cc3)c3ccc4c(c3)C(C)(C)c3ccccc3-4)ccc21] 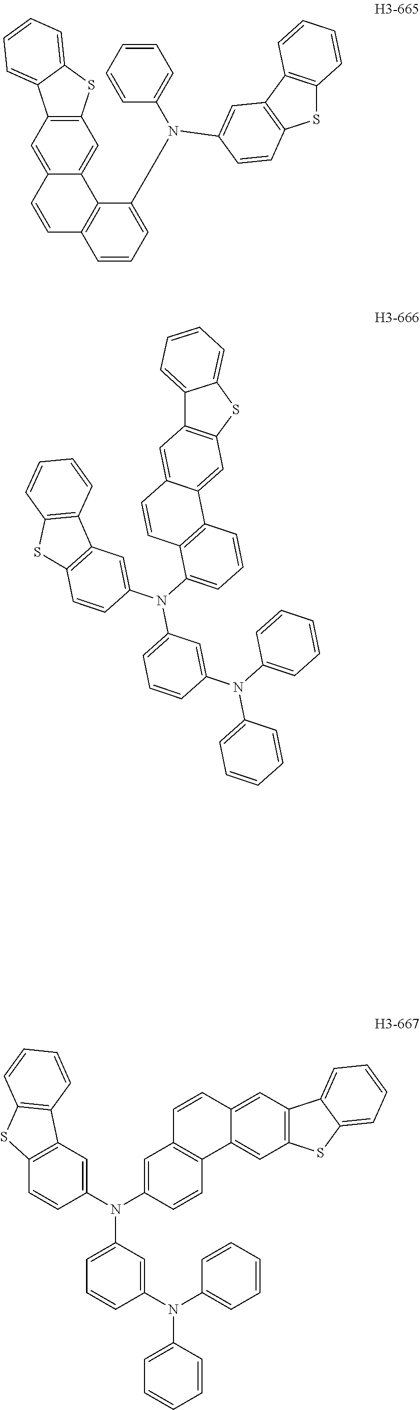 [SMILES: c1ccc(N(c2ccc3sc4ccccc4c3c2)c2cccc3ccc4cc5c(cc4c23)sc2ccccc25)cc1 ; c1ccc(N(c2ccccc2)c2cccc(N(c3ccc4sc5ccccc5c4c3)c3cccc4c3ccc3cc5c(cc34)sc3ccccc35)c2)cc1 ; c1ccc(N(c2ccccc2)c2cccc(N(c3ccc4c(ccc5cc6c(cc54)sc4ccccc46)c3)c3ccc4sc5ccccc5c4c3)c2)cc1] 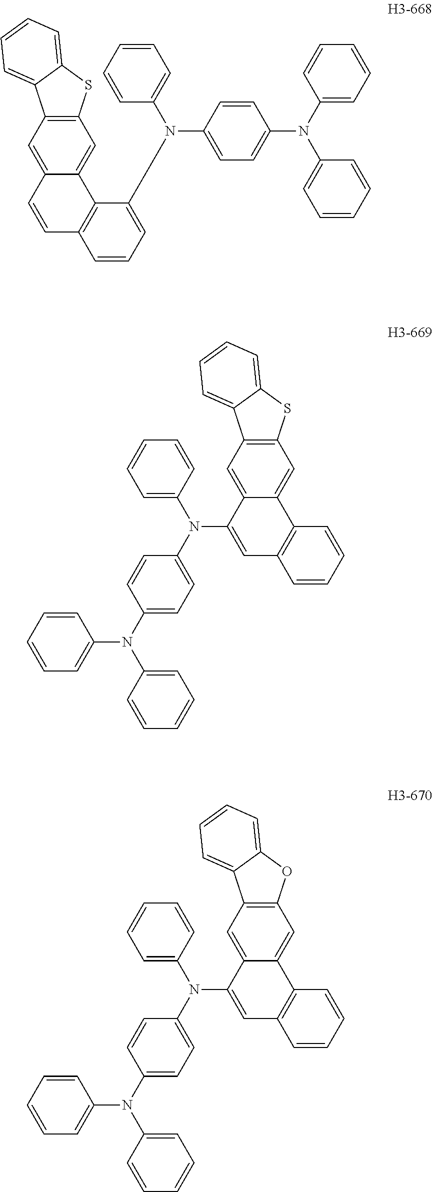 [SMILES: c1ccc(N(c2ccccc2)c2ccc(N(c3ccccc3)c3cccc4ccc5cc6c(cc5c34)sc3ccccc36)cc2)cc1 ; c1ccc(N(c2ccccc2)c2ccc(N(c3ccccc3)c3cc4ccccc4c4cc5sc6ccccc6c5cc34)cc2)cc1 ; c1ccc(N(c2ccccc2)c2ccc(N(c3ccccc3)c3cc4ccccc4c4cc5oc6ccccc6c5cc34)cc2)cc1] 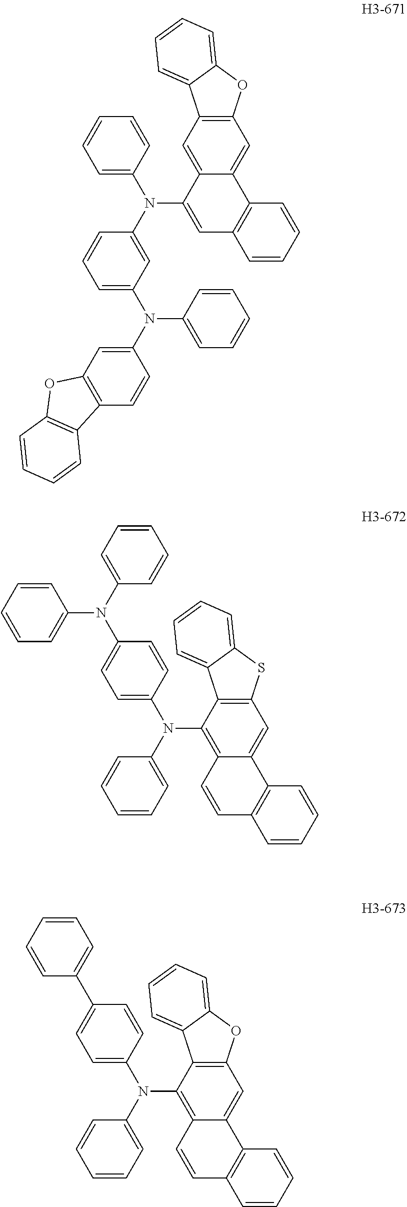 [SMILES: c1ccc(N(c2cccc(N(c3ccccc3)c3cc4ccccc4c4cc5oc6ccccc6c5cc34)c2)c2ccc3c(c2)oc2ccccc23)cc1 ; c1ccc(N(c2ccccc2)c2ccc(N(c3ccccc3)c3c4ccc5ccccc5c4cc4sc5ccccc5c34)cc2)cc1 ; c1ccc(-c2ccc(N(c3ccccc3)c3c4ccc5ccccc5c4cc4oc5ccccc5c34)cc2)cc1] 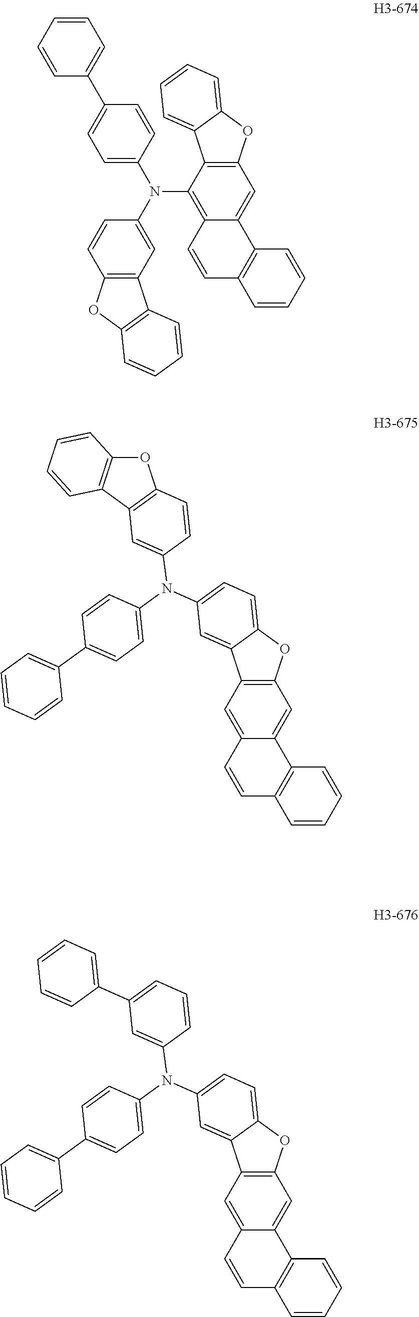 [SMILES: c1ccc(-c2ccc(N(c3ccc4oc5ccccc5c4c3)c3c4ccc5ccccc5c4cc4oc5ccccc5c34)cc2)cc1 ; c1ccc(-c2ccc(N(c3ccc4oc5ccccc5c4c3)c3ccc4oc5cc6c(ccc7ccccc76)cc5c4c3)cc2)cc1 ; c1ccc(-c2ccc(N(c3cccc(-c4ccccc4)c3)c3ccc4oc5cc6c(ccc7ccccc76)cc5c4c3)cc2)cc1] 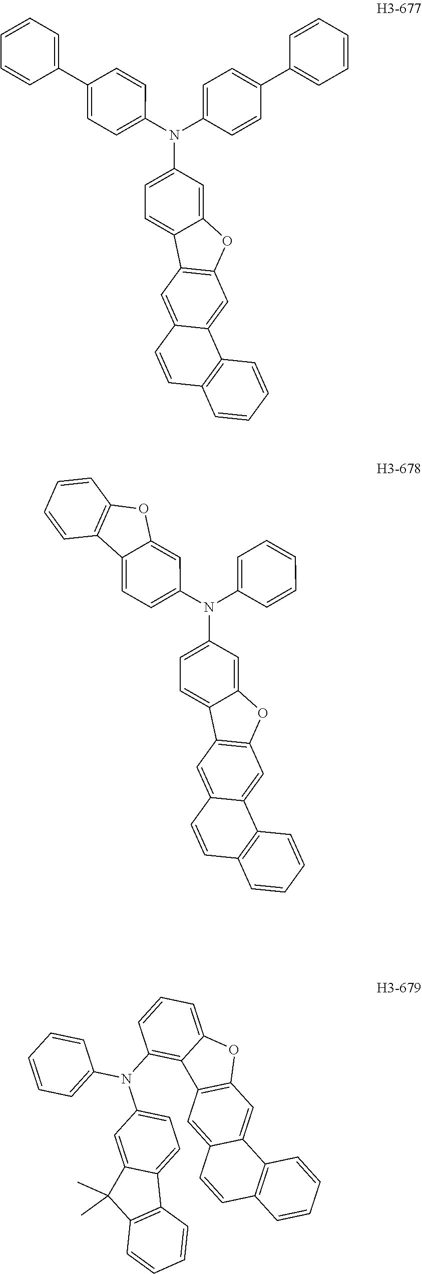 [SMILES: c1ccc(-c2ccc(N(c3ccc(-c4ccccc4)cc3)c3ccc4c(c3)oc3cc5c(ccc6ccccc65)cc34)cc2)cc1 ; c1ccc(N(c2ccc3c(c2)oc2ccccc23)c2ccc3c(c2)oc2cc4c(ccc5ccccc54)cc23)cc1 ; CC1(C)c2ccccc2-c2ccc(N(c3ccccc3)c3cccc4oc5cc6c(ccc7ccccc76)cc5c34)cc21] 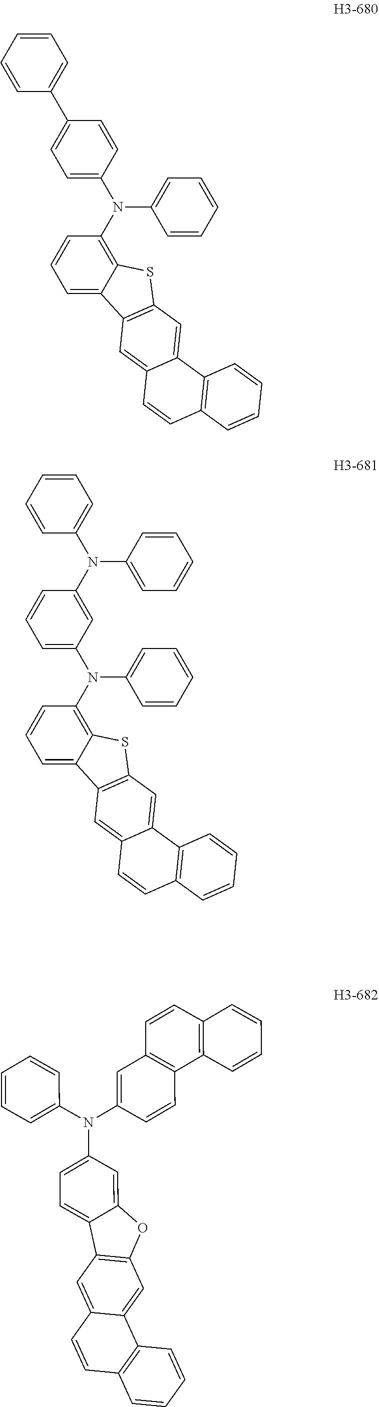 [SMILES: c1ccc(-c2ccc(N(c3ccccc3)c3cccc4c3sc3cc5c(ccc6ccccc65)cc34)cc2)cc1 ; c1ccc(N(c2ccccc2)c2cccc(N(c3ccccc3)c3cccc4c3sc3cc5c(ccc6ccccc65)cc34)c2)cc1 ; c1ccc(N(c2ccc3c(ccc4ccccc43)c2)c2ccc3c(c2)oc2cc4c(ccc5ccccc54)cc23)cc1] 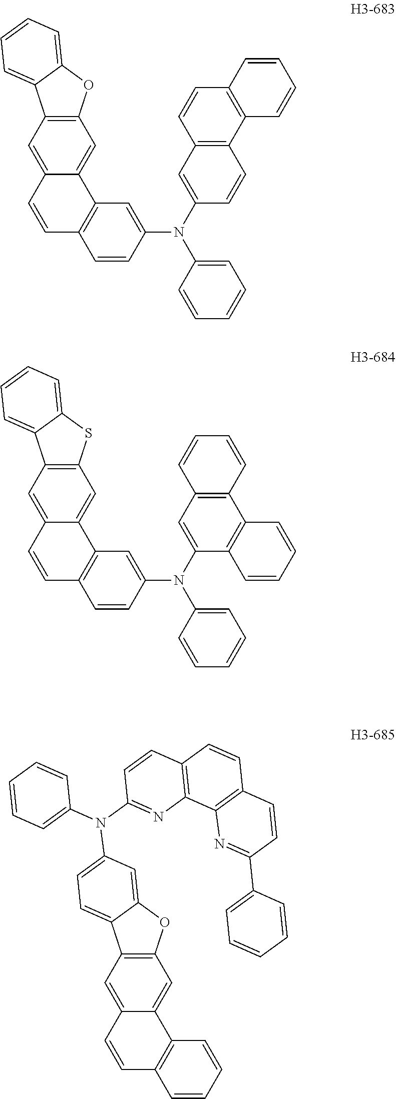 [SMILES: c1ccc(N(c2ccc3c(ccc4ccccc43)c2)c2ccc3ccc4cc5c(cc4c3c2)oc2ccccc25)cc1 ; c1ccc(N(c2ccc3ccc4cc5c(cc4c3c2)sc2ccccc25)c2cc3ccccc3c3ccccc23)cc1 ; c1ccc(-c2ccc3ccc4ccc(N(c5ccccc5)c5ccc6c(c5)oc5cc7c(ccc8ccccc87)cc56)nc4c3n2)cc1] 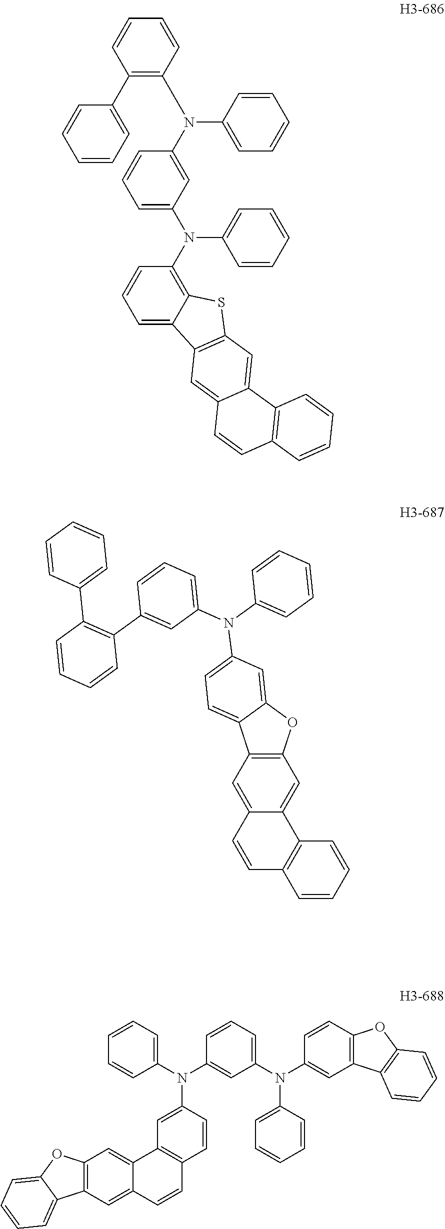 [SMILES: c1ccc(-c2ccccc2N(c2ccccc2)c2cccc(N(c3ccccc3)c3cccc4c3sc3cc5c(ccc6ccccc65)cc34)c2)cc1 ; c1ccc(-c2ccccc2-c2cccc(N(c3ccccc3)c3ccc4c(c3)oc3cc5c(ccc6ccccc65)cc34)c2)cc1 ; c1ccc(N(c2cccc(N(c3ccccc3)c3ccc4oc5ccccc5c4c3)c2)c2ccc3ccc4cc5c(cc4c3c2)oc2ccccc25)cc1] 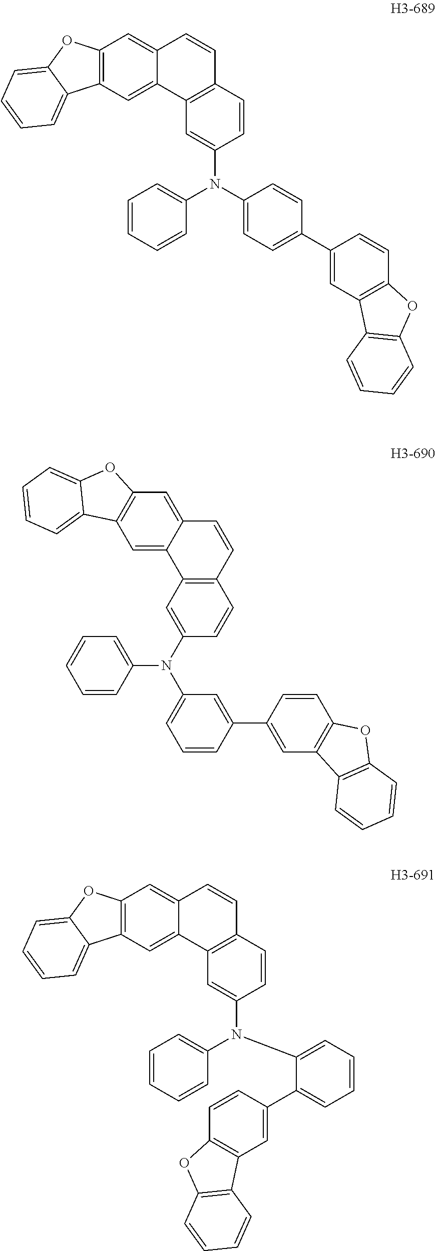 [SMILES: c1ccc(N(c2ccc(-c3ccc4oc5ccccc5c4c3)cc2)c2ccc3ccc4cc5oc6ccccc6c5cc4c3c2)cc1 ; c1ccc(N(c2cccc(-c3ccc4oc5ccccc5c4c3)c2)c2ccc3ccc4cc5oc6ccccc6c5cc4c3c2)cc1 ; c1ccc(N(c2ccc3ccc4cc5oc6ccccc6c5cc4c3c2)c2ccccc2-c2ccc3oc4ccccc4c3c2)cc1] 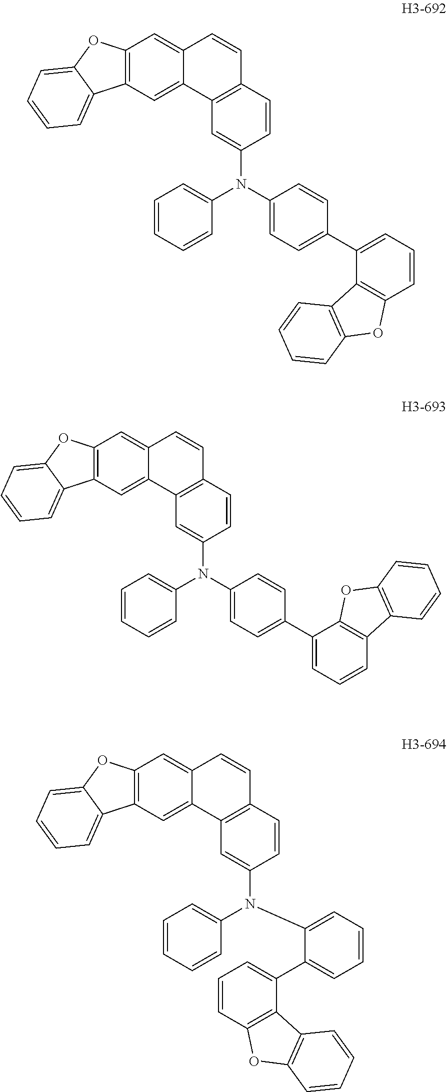 [SMILES: c1ccc(N(c2ccc(-c3cccc4oc5ccccc5c34)cc2)c2ccc3ccc4cc5oc6ccccc6c5cc4c3c2)cc1 ; c1ccc(N(c2ccc(-c3cccc4c3oc3ccccc34)cc2)c2ccc3ccc4cc5oc6ccccc6c5cc4c3c2)cc1 ; c1ccc(N(c2ccc3ccc4cc5oc6ccccc6c5cc4c3c2)c2ccccc2-c2cccc3oc4ccccc4c23)cc1] 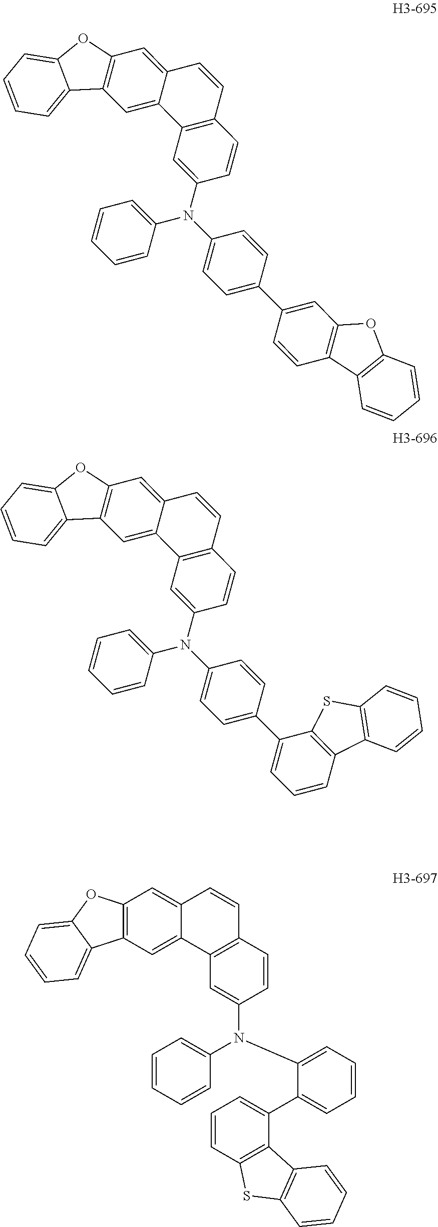 [SMILES: c1ccc(N(c2ccc(-c3ccc4c(c3)oc3ccccc34)cc2)c2ccc3ccc4cc5oc6ccccc6c5cc4c3c2)cc1 ; c1ccc(N(c2ccc(-c3cccc4c3sc3ccccc34)cc2)c2ccc3ccc4cc5oc6ccccc6c5cc4c3c2)cc1 ; c1ccc(N(c2ccc3ccc4cc5oc6ccccc6c5cc4c3c2)c2ccccc2-c2cccc3sc4ccccc4c23)cc1] 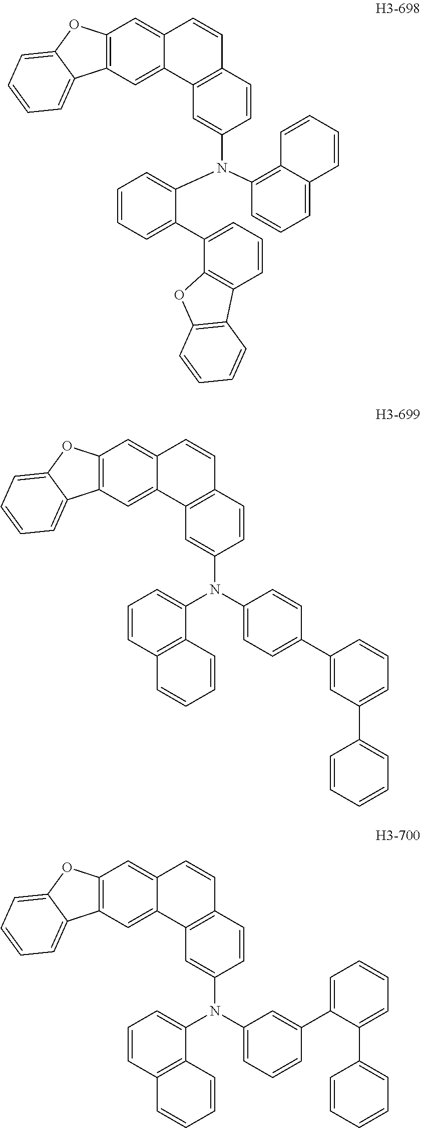 [SMILES: c1ccc(N(c2ccc3ccc4cc5oc6ccccc6c5cc4c3c2)c2cccc3ccccc23)c(-c2cccc3c2oc2ccccc23)c1 ; c1ccc(-c2cccc(-c3ccc(N(c4ccc5ccc6cc7oc8ccccc8c7cc6c5c4)c4cccc5ccccc45)cc3)c2)cc1 ; c1ccc(-c2ccccc2-c2cccc(N(c3ccc4ccc5cc6oc7ccccc7c6cc5c4c3)c3cccc4ccccc34)c2)cc1] 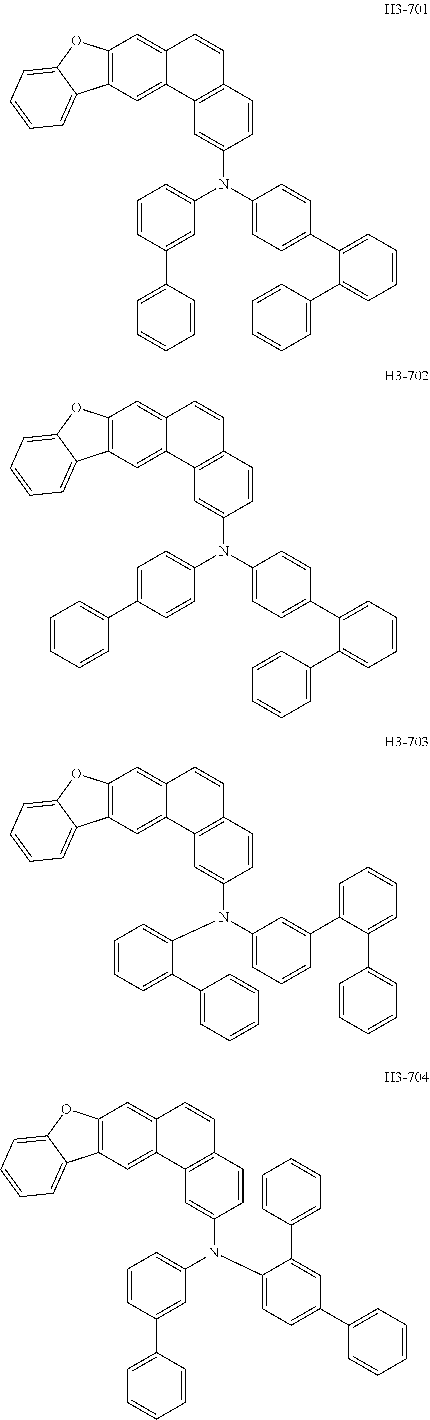 [SMILES: c1ccc(-c2cccc(N(c3ccc(-c4ccccc4-c4ccccc4)cc3)c3ccc4ccc5cc6oc7ccccc7c6cc5c4c3)c2)cc1 ; c1ccc(-c2ccc(N(c3ccc(-c4ccccc4-c4ccccc4)cc3)c3ccc4ccc5cc6oc7ccccc7c6cc5c4c3)cc2)cc1 ; c1ccc(-c2ccccc2-c2cccc(N(c3ccc4ccc5cc6oc7ccccc7c6cc5c4c3)c3ccccc3-c3ccccc3)c2)cc1 ; c1ccc(-c2cccc(N(c3ccc4ccc5cc6oc7ccccc7c6cc5c4c3)c3ccc(-c4ccccc4)cc3-c3ccccc3)c2)cc1] 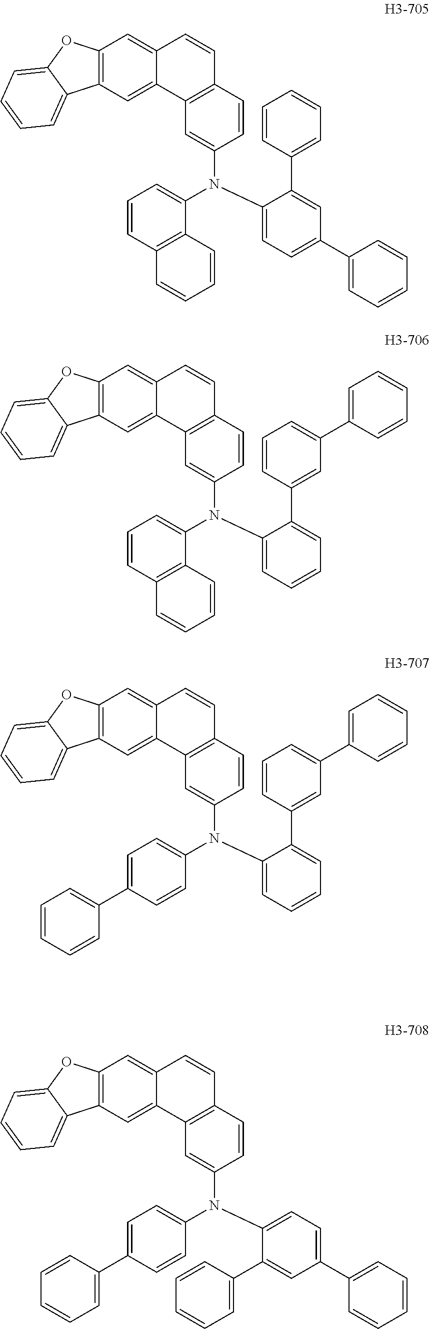 [SMILES: c1ccc(-c2ccc(N(c3ccc4ccc5cc6oc7ccccc7c6cc5c4c3)c3cccc4ccccc34)c(-c3ccccc3)c2)cc1 ; c1ccc(-c2cccc(-c3ccccc3N(c3ccc4ccc5cc6oc7ccccc7c6cc5c4c3)c3cccc4ccccc34)c2)cc1 ; c1ccc(-c2ccc(N(c3ccc4ccc5cc6oc7ccccc7c6cc5c4c3)c3ccccc3-c3cccc(-c4ccccc4)c3)cc2)cc1 ; c1ccc(-c2ccc(N(c3ccc4ccc5cc6oc7ccccc7c6cc5c4c3)c3ccc(-c4ccccc4)cc3-c3ccccc3)cc2)cc1] 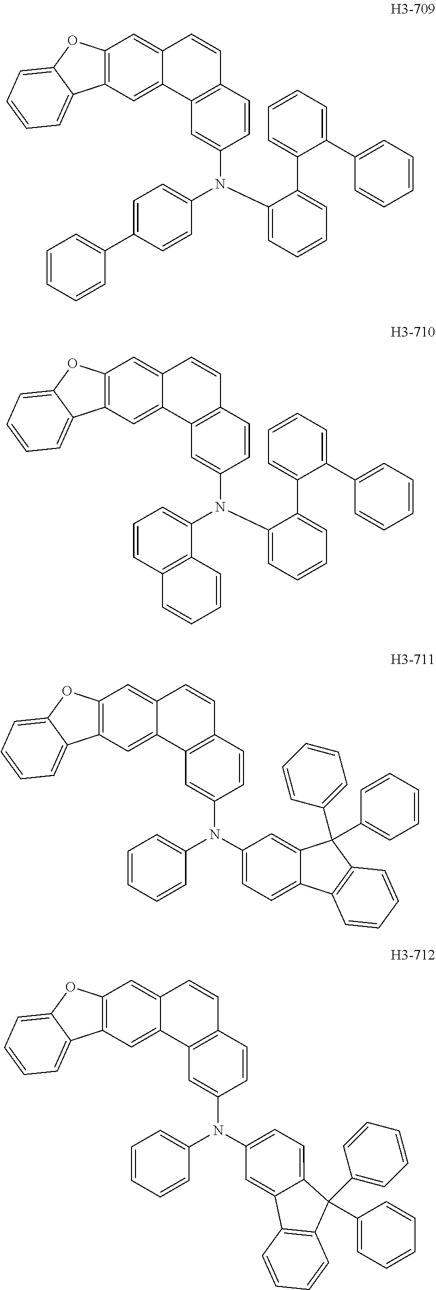 [SMILES: c1ccc(-c2ccc(N(c3ccc4ccc5cc6oc7ccccc7c6cc5c4c3)c3ccccc3-c3ccccc3-c3ccccc3)cc2)cc1 ; c1ccc(-c2ccccc2-c2ccccc2N(c2ccc3ccc4cc5oc6ccccc6c5cc4c3c2)c2cccc3ccccc23)cc1 ; c1ccc(N(c2ccc3c(c2)C(c2ccccc2)(c2ccccc2)c2ccccc2-3)c2ccc3ccc4cc5oc6ccccc6c5cc4c3c2)cc1 ; c1ccc(N(c2ccc3c(c2)-c2ccccc2C3(c2ccccc2)c2ccccc2)c2ccc3ccc4cc5oc6ccccc6c5cc4c3c2)cc1] 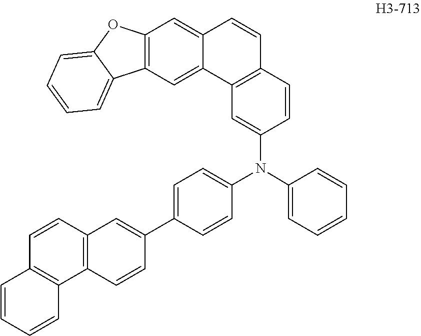 [SMILES: c1ccc(N(c2ccc(-c3ccc4c(ccc5ccccc54)c3)cc2)c2ccc3ccc4cc5oc6ccccc6c5cc4c3c2)cc1] 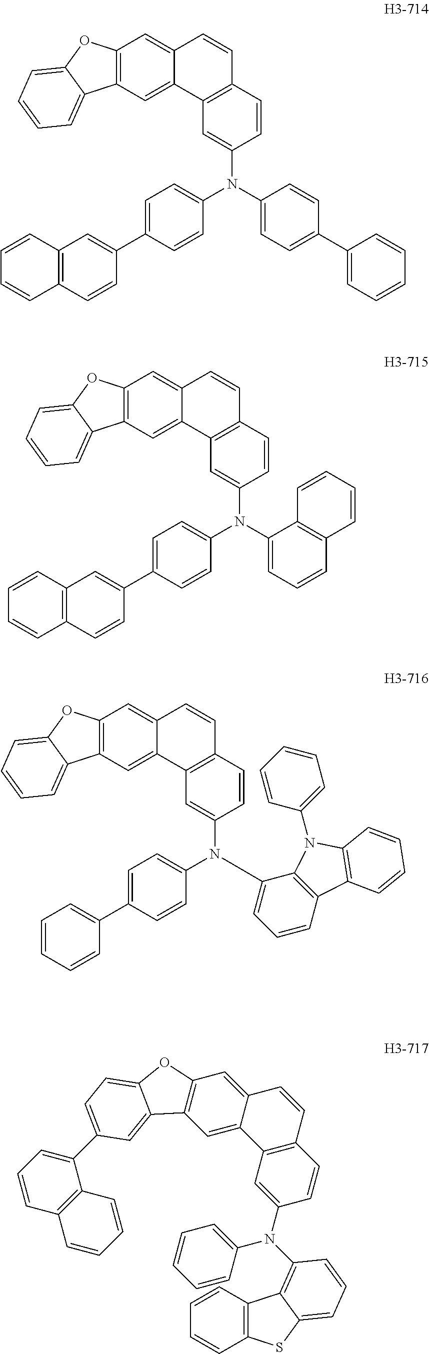 [SMILES: c1ccc(-c2ccc(N(c3ccc(-c4ccc5ccccc5c4)cc3)c3ccc4ccc5cc6oc7ccccc7c6cc5c4c3)cc2)cc1 ; c1ccc2cc(-c3ccc(N(c4ccc5ccc6cc7oc8ccccc8c7cc6c5c4)c4cccc5ccccc45)cc3)ccc2c1 ; c1ccc(-c2ccc(N(c3ccc4ccc5cc6oc7ccccc7c6cc5c4c3)c3cccc4c5ccccc5n(-c5ccccc5)c34)cc2)cc1 ; c1ccc(N(c2ccc3ccc4cc5oc6ccc(-c7cccc8ccccc78)cc6c5cc4c3c2)c2cccc3sc4ccccc4c23)cc1] 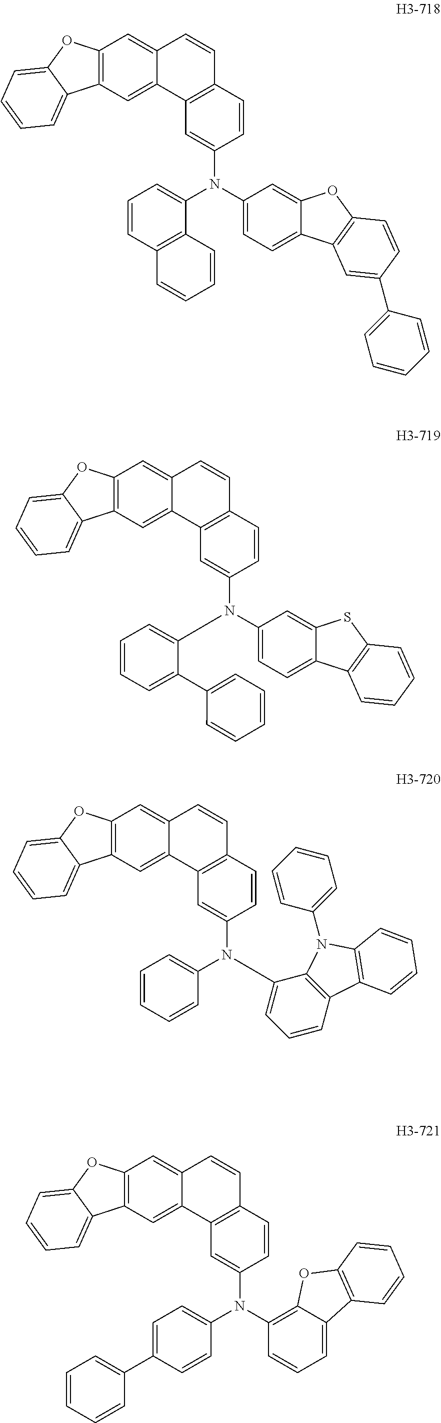 [SMILES: c1ccc(-c2ccc3oc4cc(N(c5ccc6ccc7cc8oc9ccccc9c8cc7c6c5)c5cccc6ccccc56)ccc4c3c2)cc1 ; c1ccc(-c2ccccc2N(c2ccc3c(c2)sc2ccccc23)c2ccc3ccc4cc5oc6ccccc6c5cc4c3c2)cc1 ; c1ccc(N(c2ccc3ccc4cc5oc6ccccc6c5cc4c3c2)c2cccc3c4ccccc4n(-c4ccccc4)c23)cc1 ; c1ccc(-c2ccc(N(c3ccc4ccc5cc6oc7ccccc7c6cc5c4c3)c3cccc4c3oc3ccccc34)cc2)cc1] 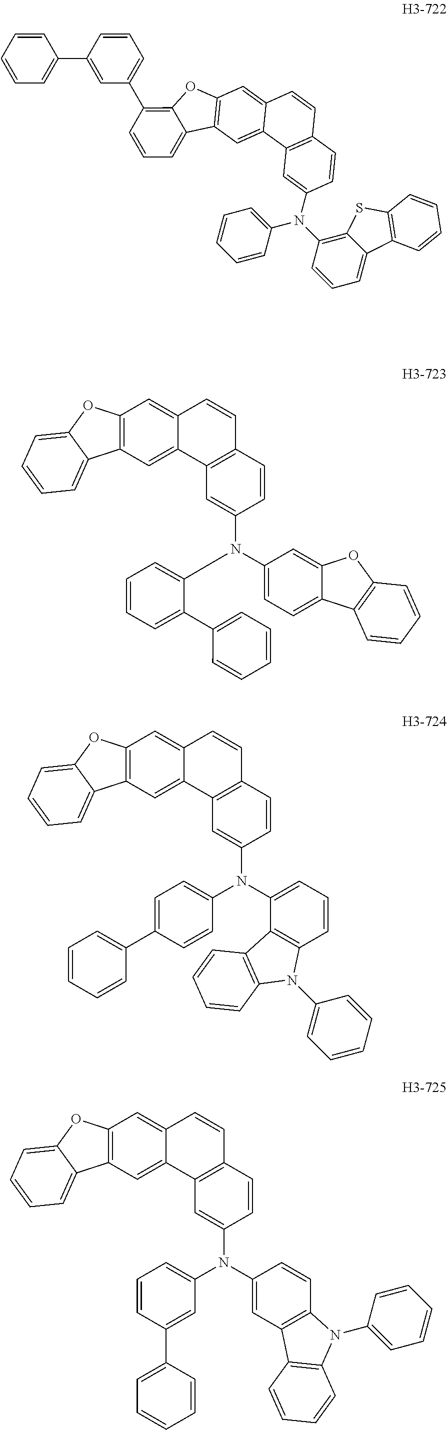 [SMILES: c1ccc(-c2cccc(-c3cccc4c3oc3cc5ccc6ccc(N(c7ccccc7)c7cccc8c7sc7ccccc78)cc6c5cc34)c2)cc1 ; c1ccc(-c2ccccc2N(c2ccc3c(c2)oc2ccccc23)c2ccc3ccc4cc5oc6ccccc6c5cc4c3c2)cc1 ; c1ccc(-c2ccc(N(c3ccc4ccc5cc6oc7ccccc7c6cc5c4c3)c3cccc4c3c3ccccc3n4-c3ccccc3)cc2)cc1 ; c1ccc(-c2cccc(N(c3ccc4ccc5cc6oc7ccccc7c6cc5c4c3)c3ccc4c(c3)c3ccccc3n4-c3ccccc3)c2)cc1] 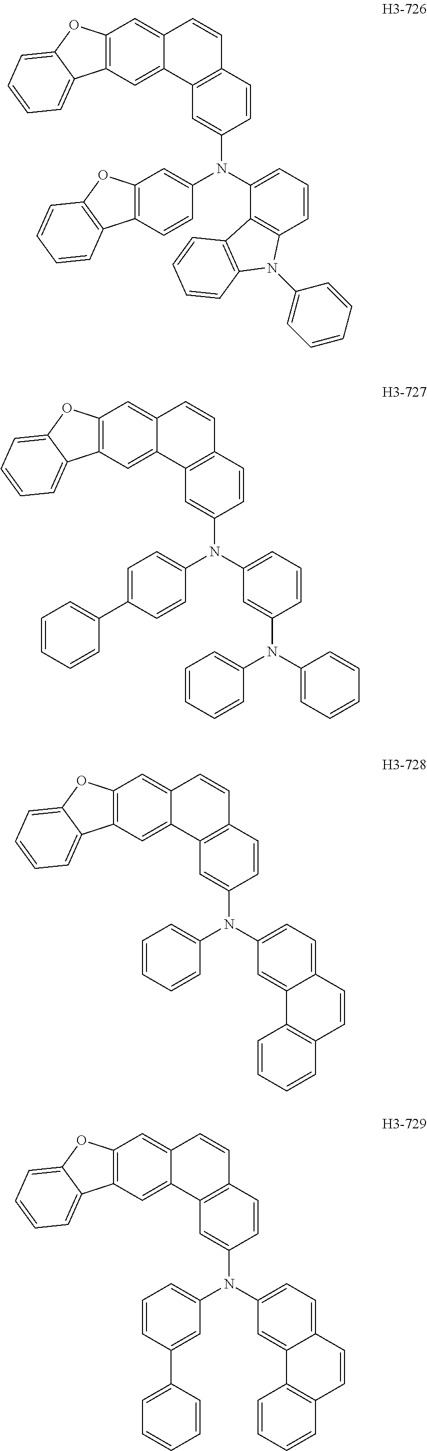 [SMILES: c1ccc(-n2c3ccccc3c3c(N(c4ccc5c(c4)oc4ccccc45)c4ccc5ccc6cc7oc8ccccc8c7cc6c5c4)cccc32)cc1 ; c1ccc(-c2ccc(N(c3cccc(N(c4ccccc4)c4ccccc4)c3)c3ccc4ccc5cc6oc7ccccc7c6cc5c4c3)cc2)cc1 ; c1ccc(N(c2ccc3ccc4ccccc4c3c2)c2ccc3ccc4cc5oc6ccccc6c5cc4c3c2)cc1 ; c1ccc(-c2cccc(N(c3ccc4ccc5ccccc5c4c3)c3ccc4ccc5cc6oc7ccccc7c6cc5c4c3)c2)cc1] 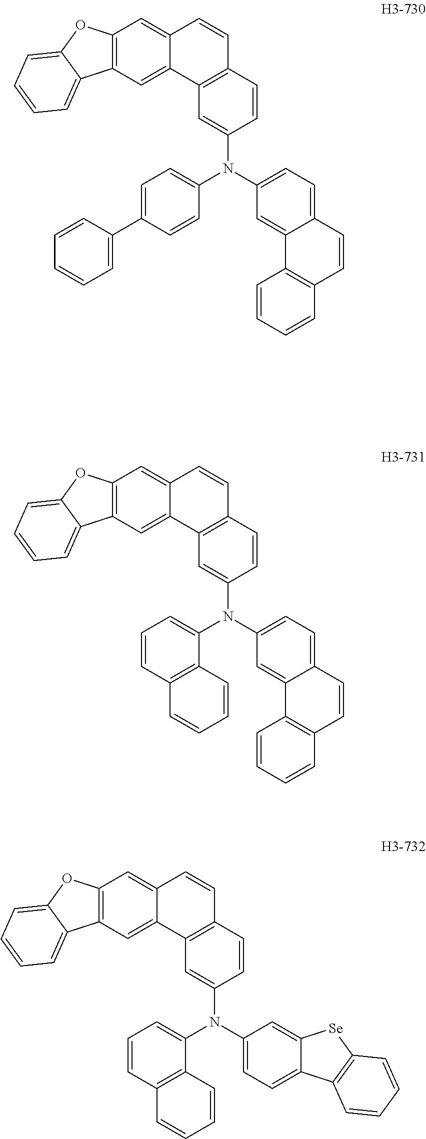 [SMILES: c1ccc(-c2ccc(N(c3ccc4ccc5ccccc5c4c3)c3ccc4ccc5cc6oc7ccccc7c6cc5c4c3)cc2)cc1 ; c1ccc2c(N(c3ccc4ccc5ccccc5c4c3)c3ccc4ccc5cc6oc7ccccc7c6cc5c4c3)cccc2c1 ; c1ccc2c(N(c3ccc4c(c3)[se]c3ccccc34)c3ccc4ccc5cc6oc7ccccc7c6cc5c4c3)cccc2c1] 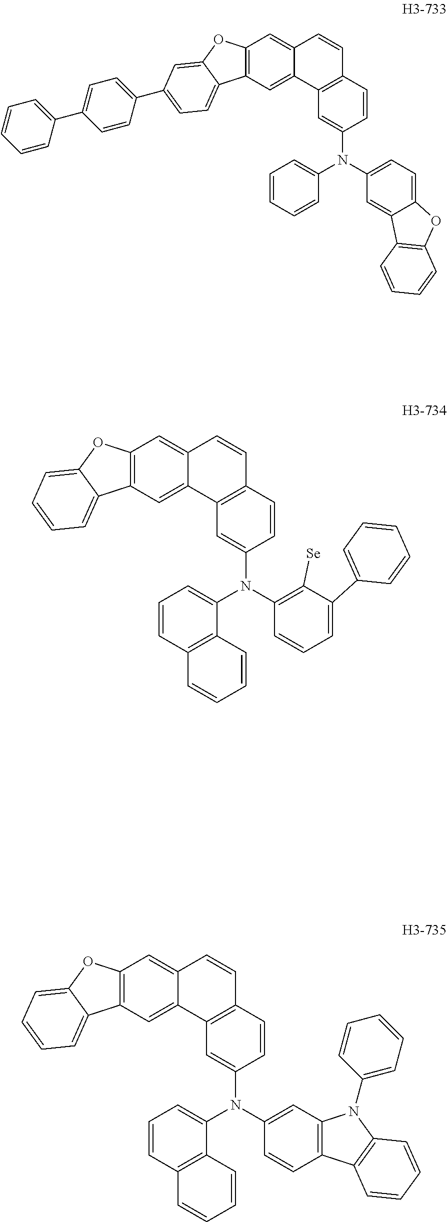 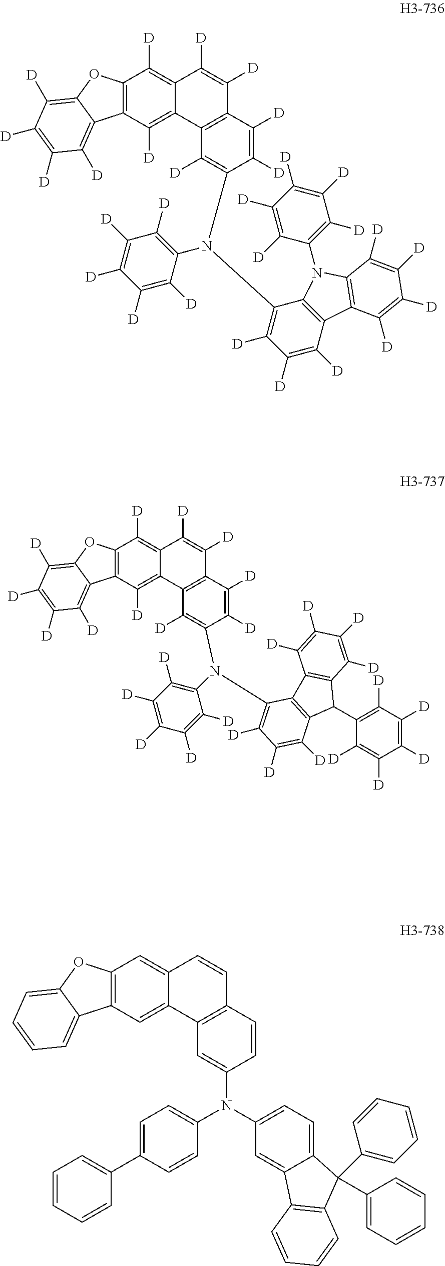 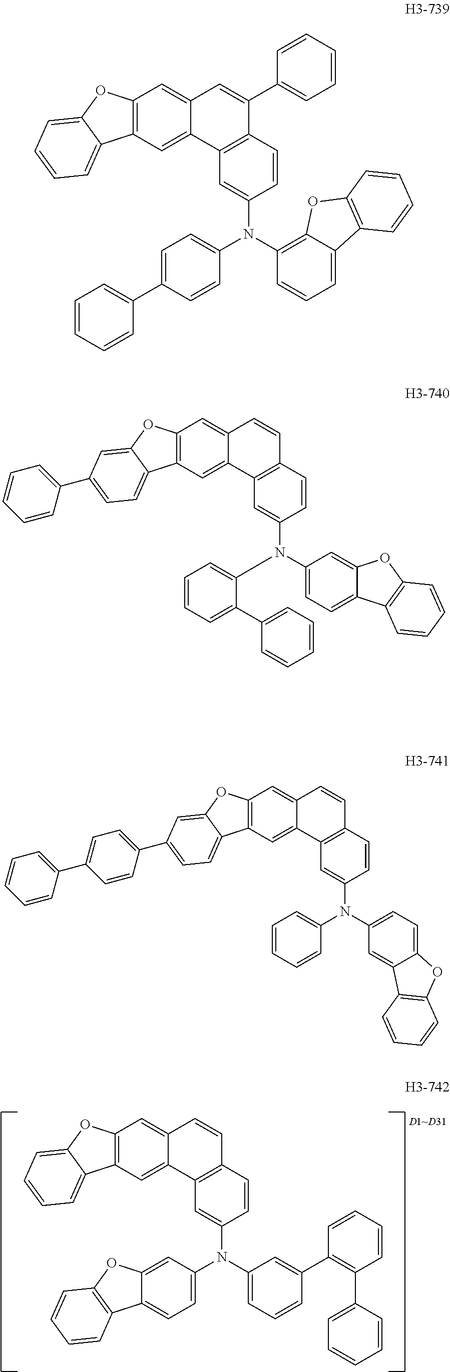 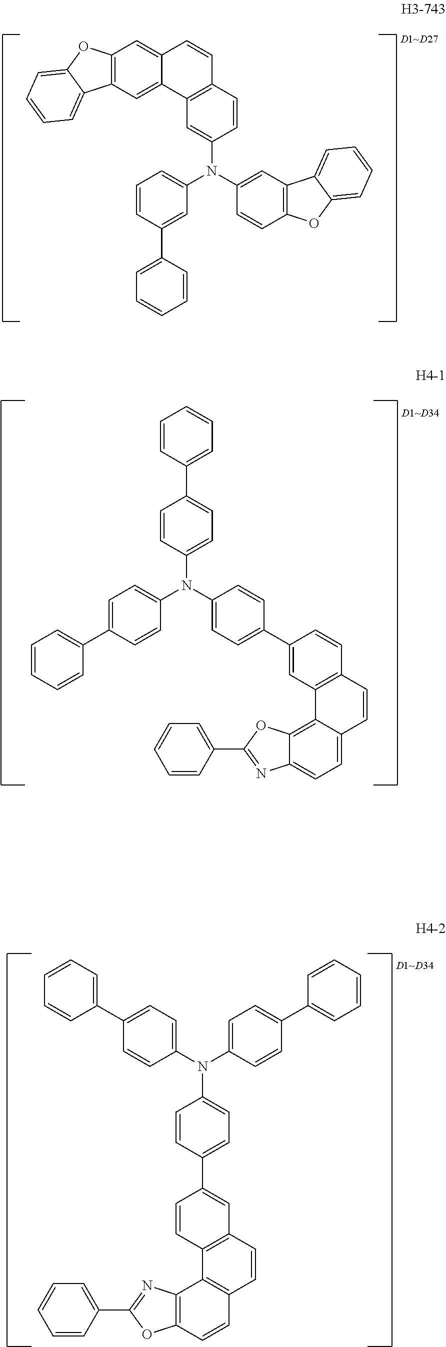 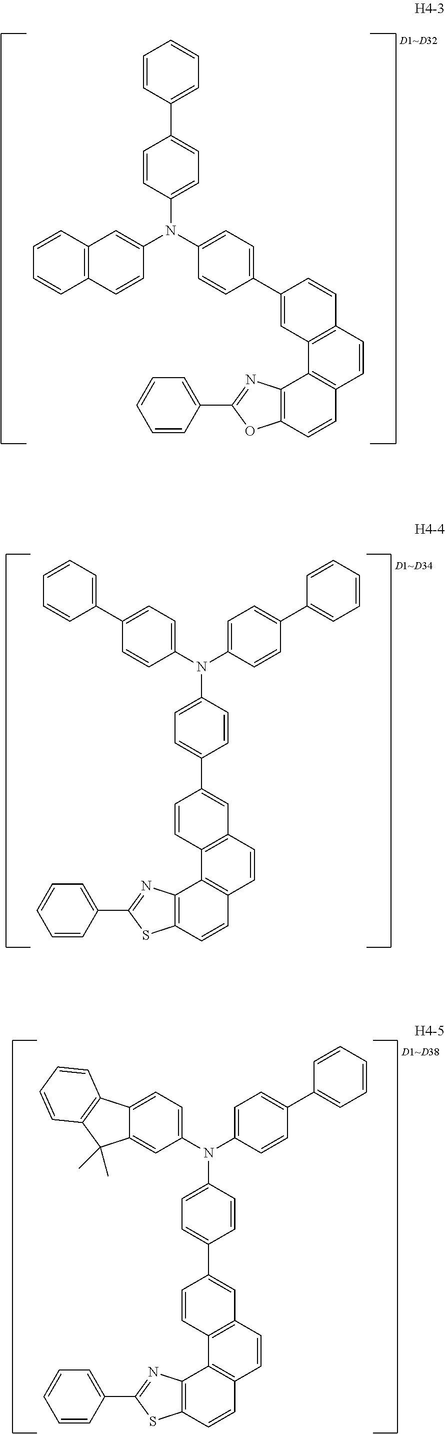 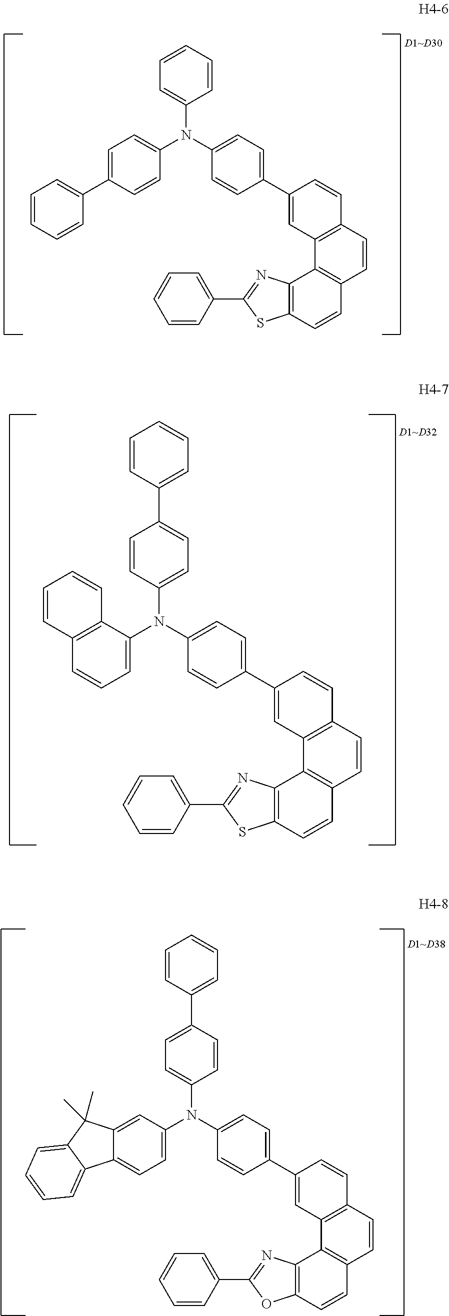 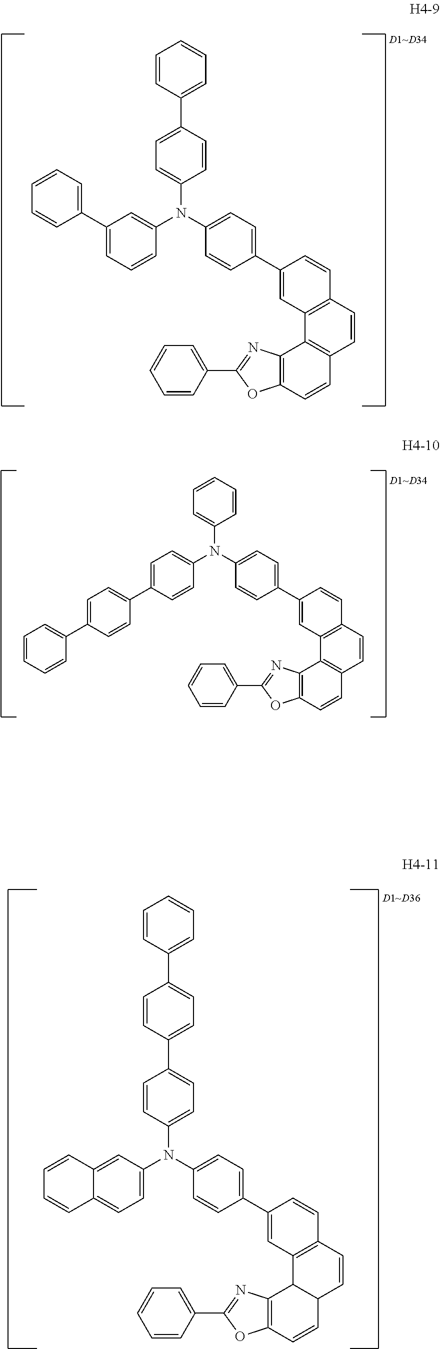 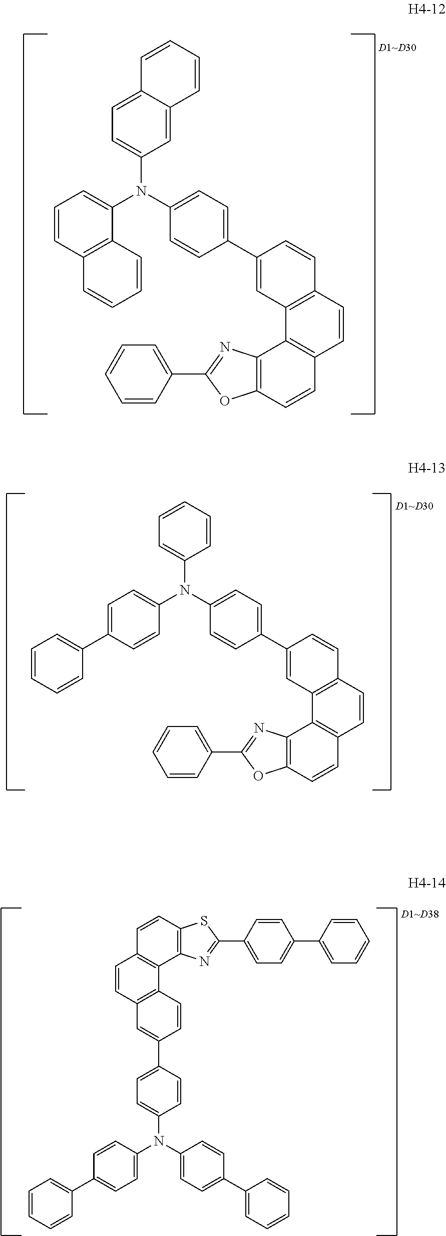 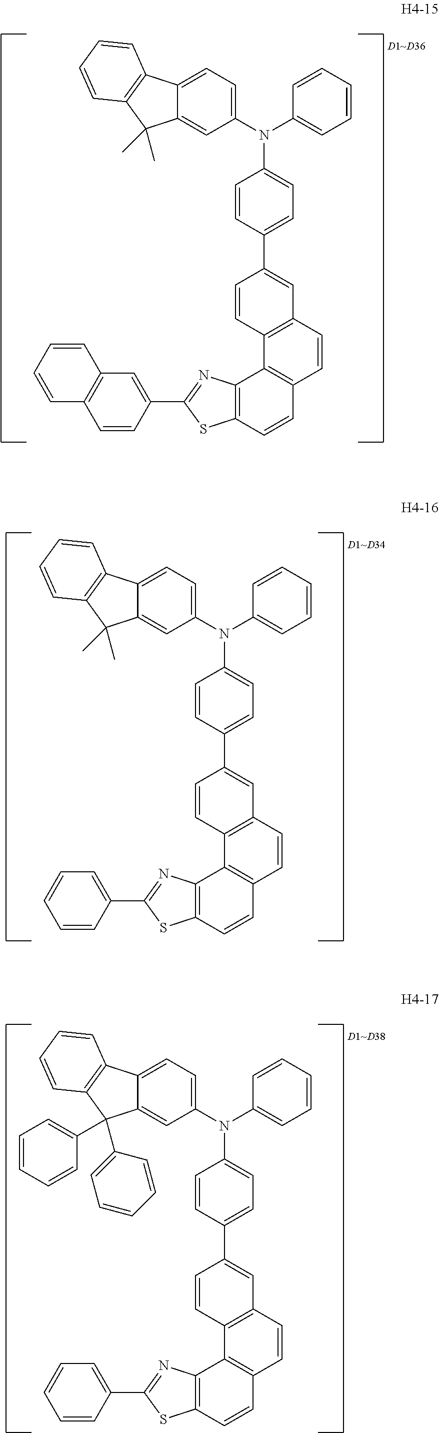 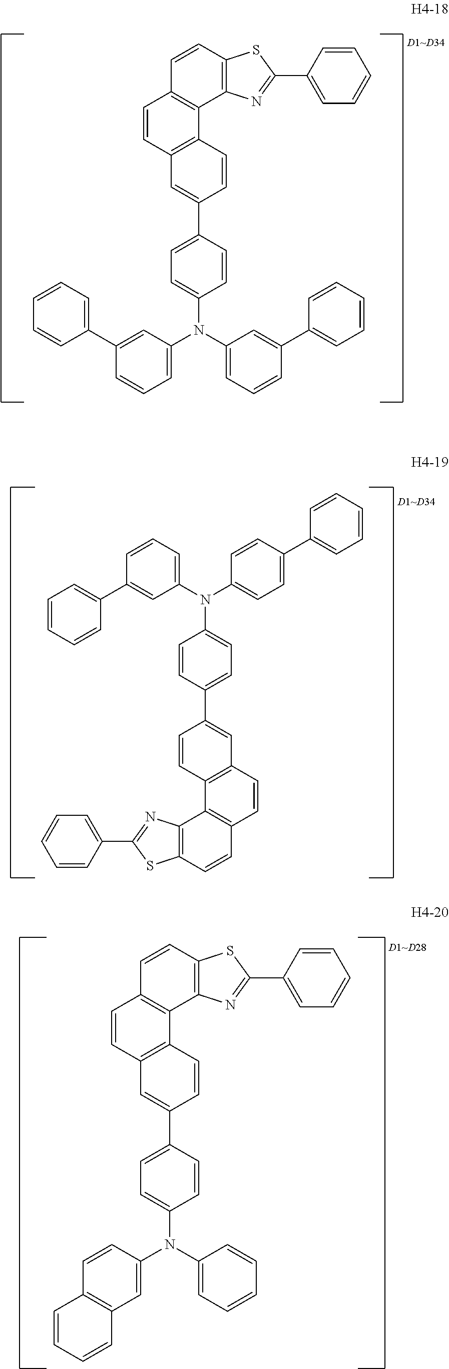 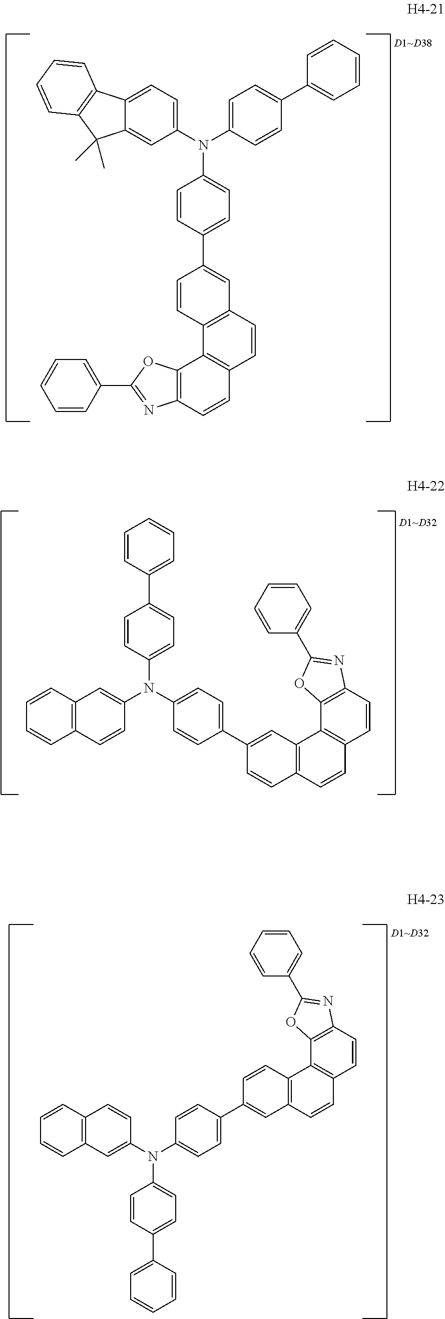 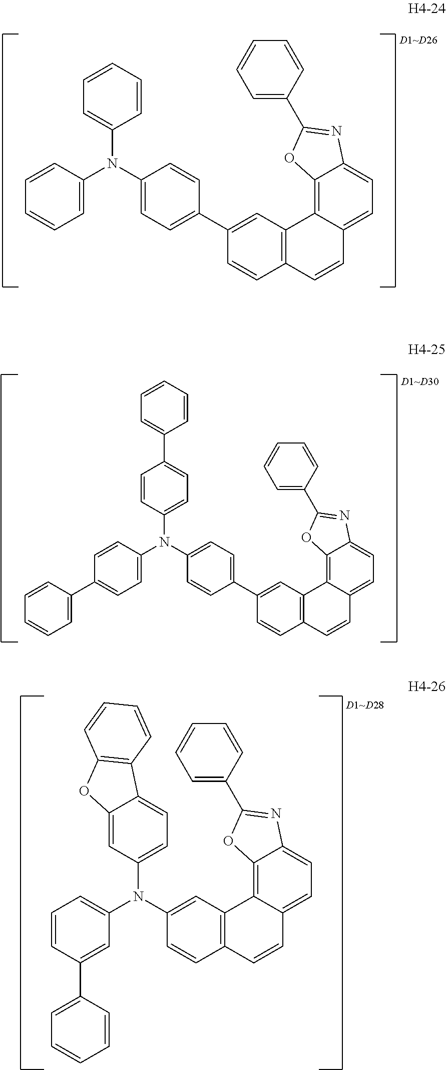 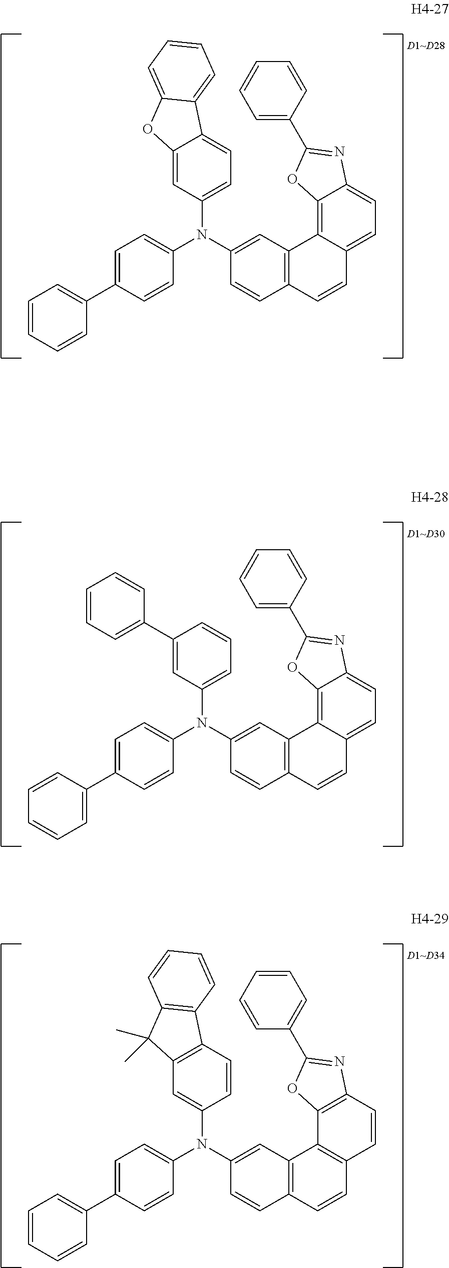 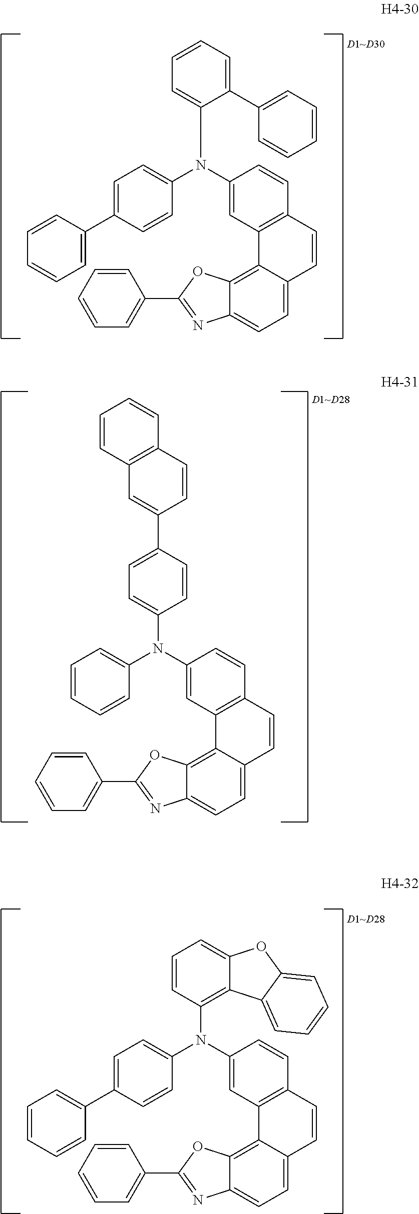 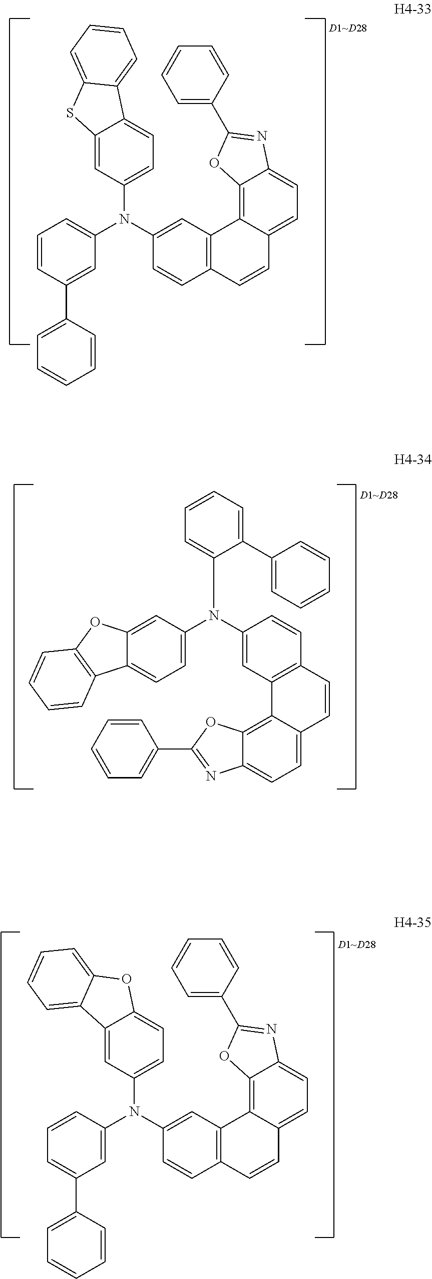 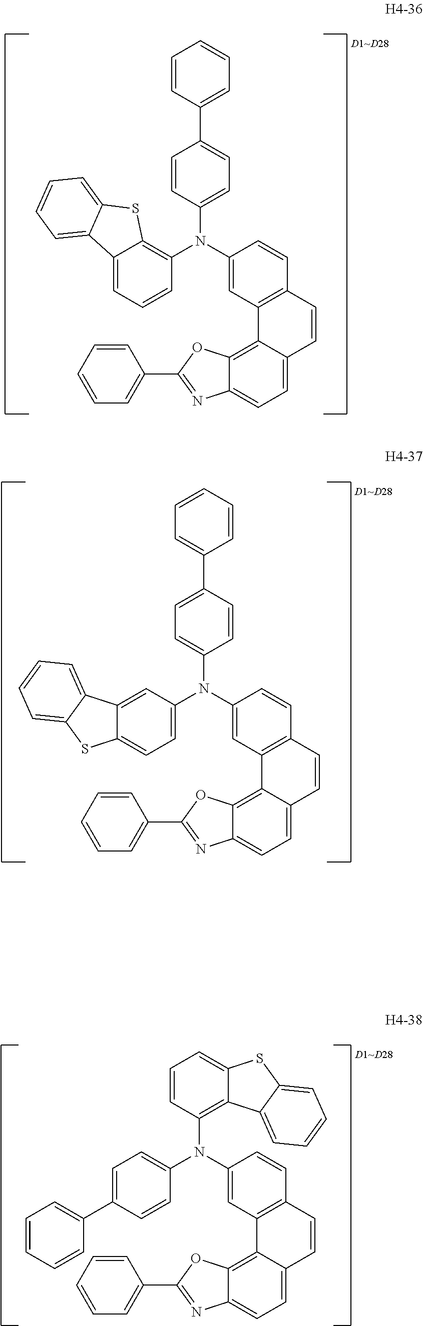 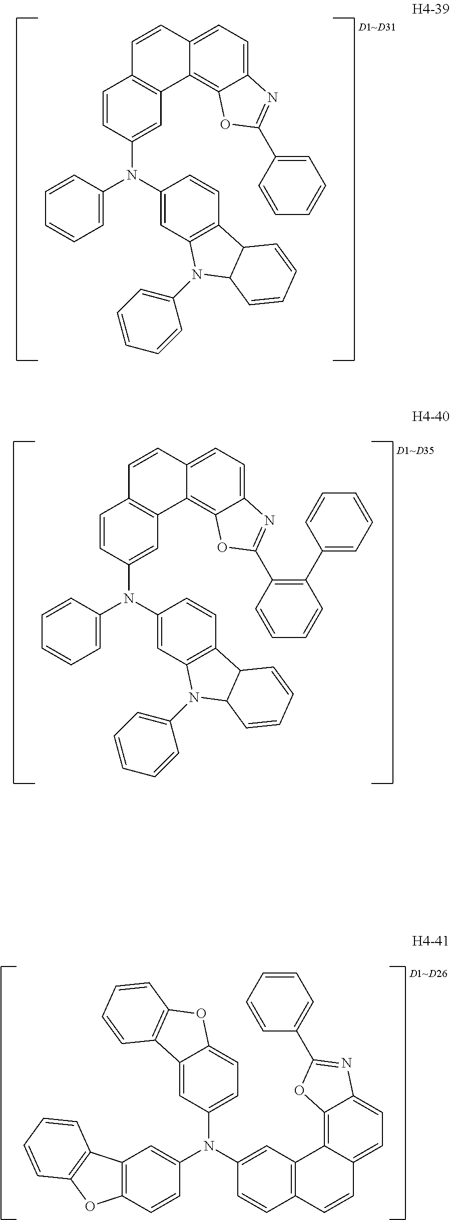 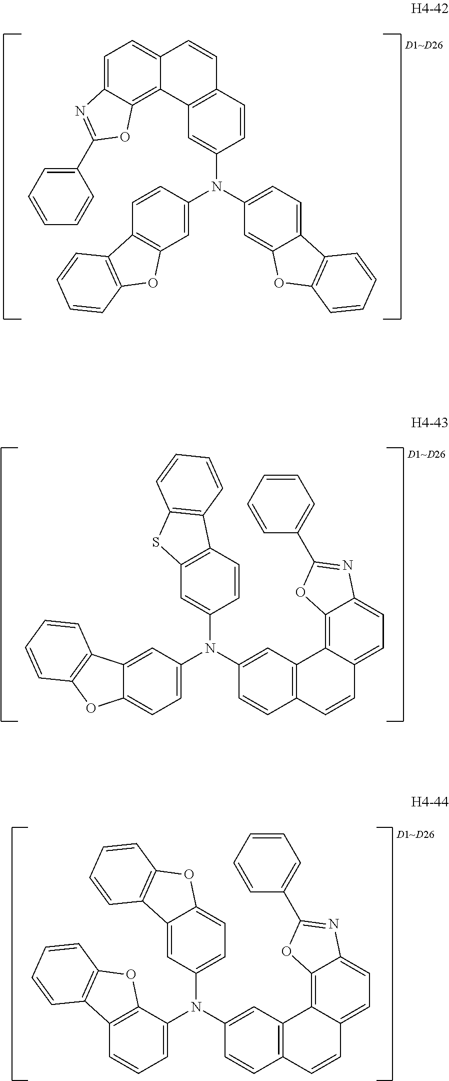 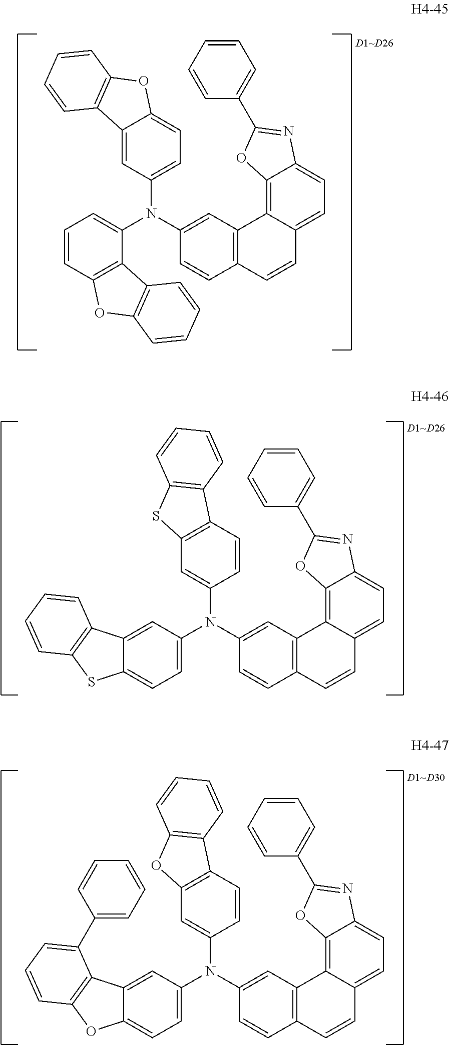 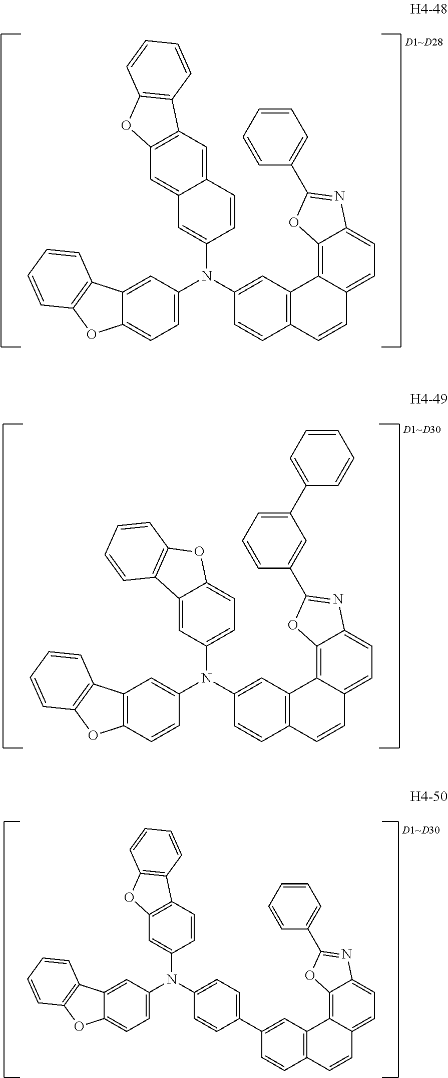 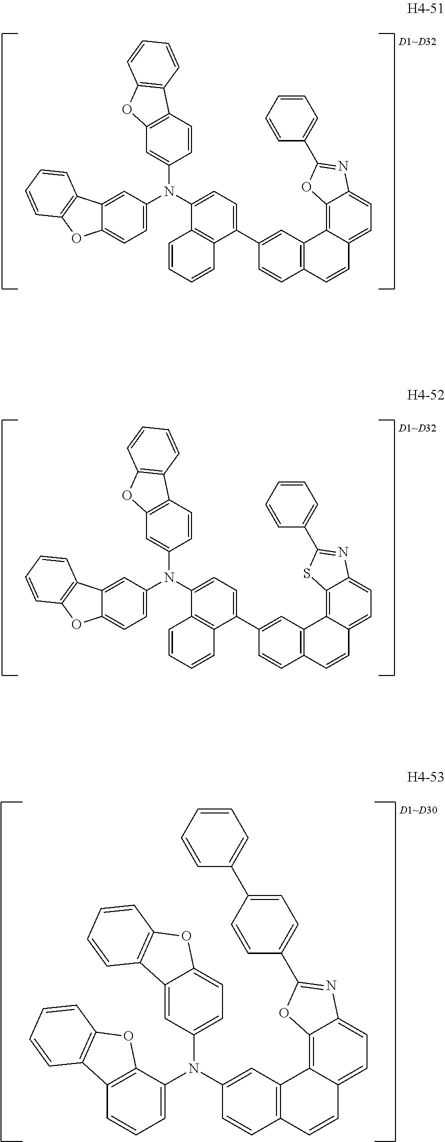 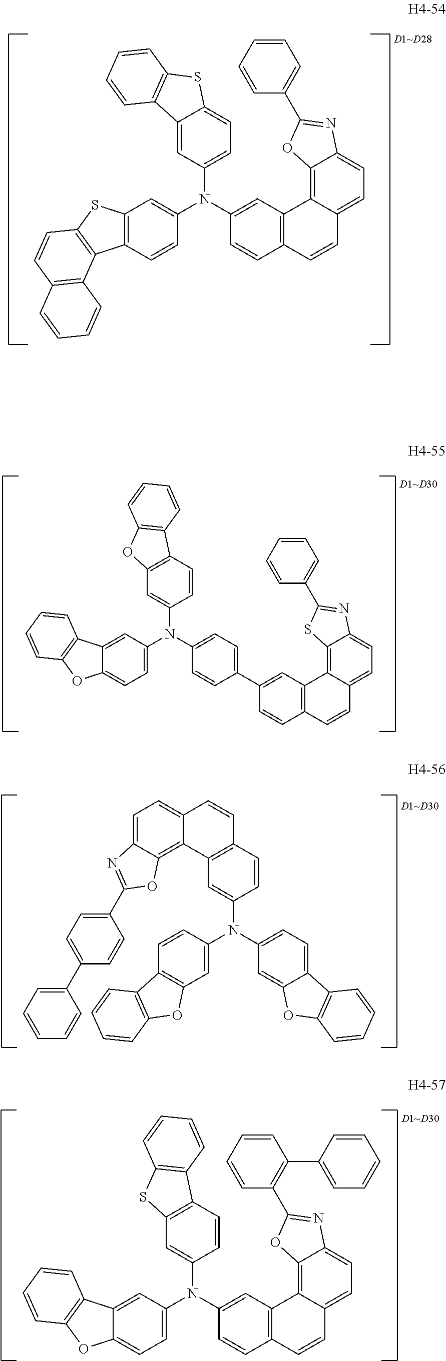 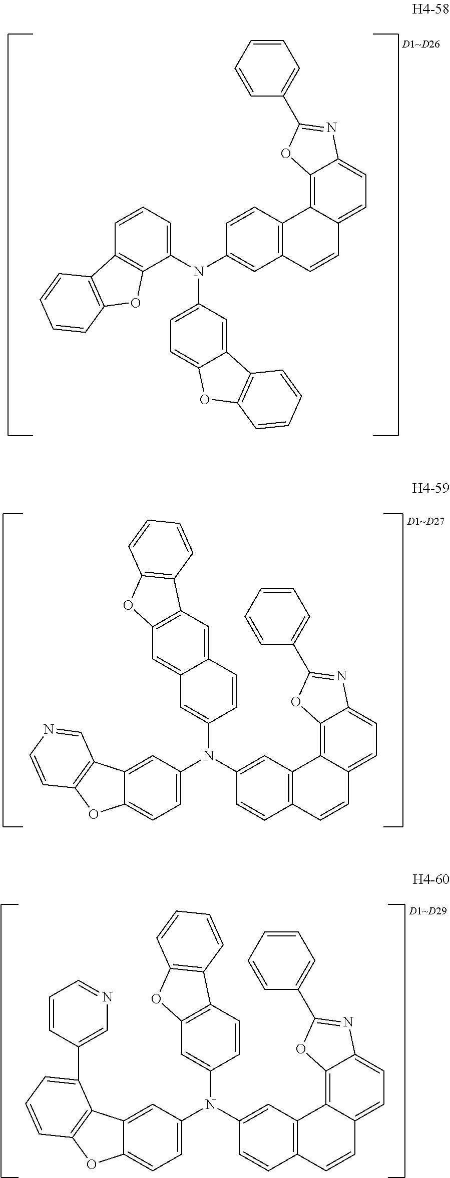 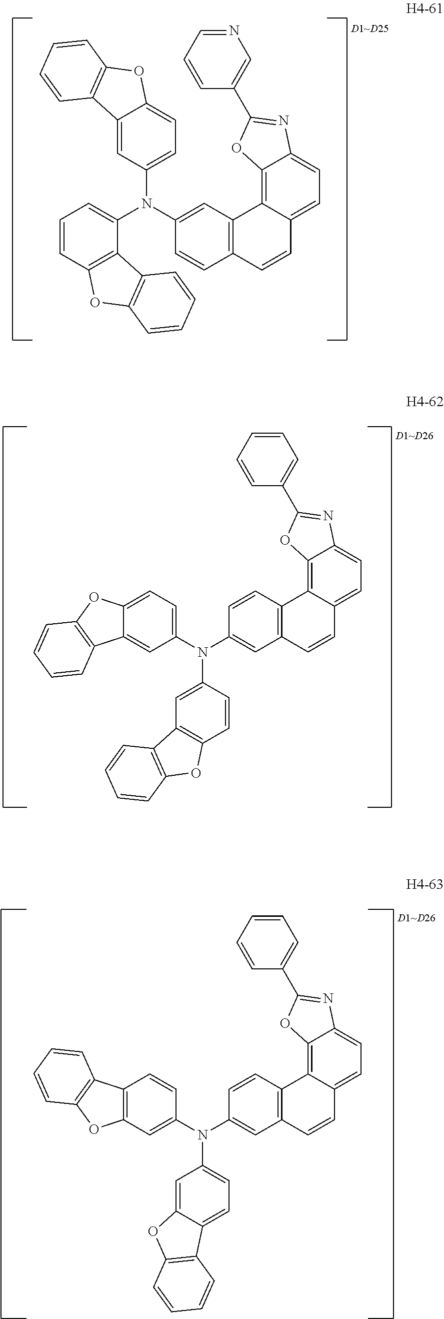 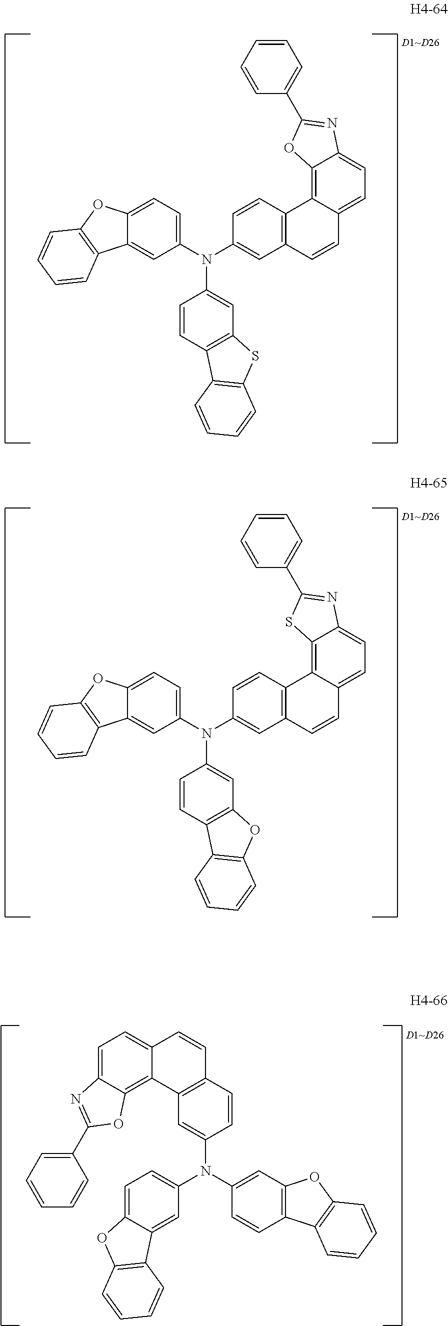 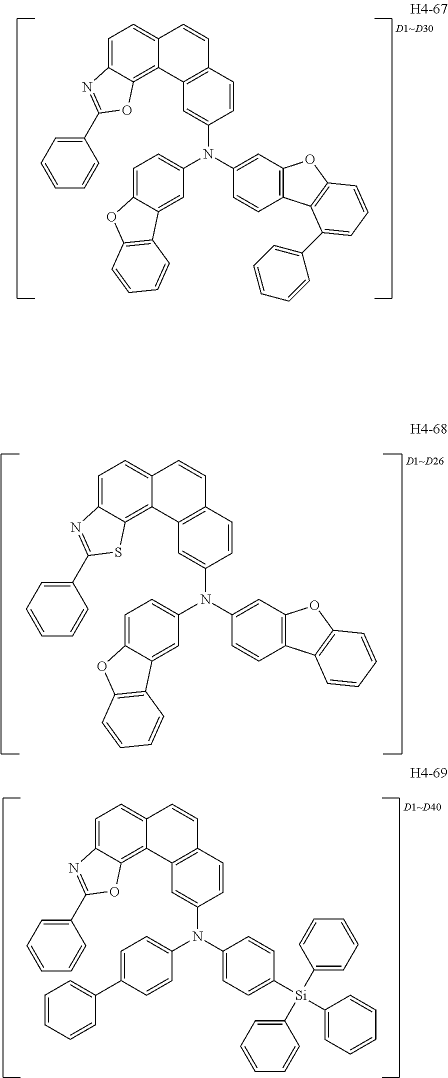 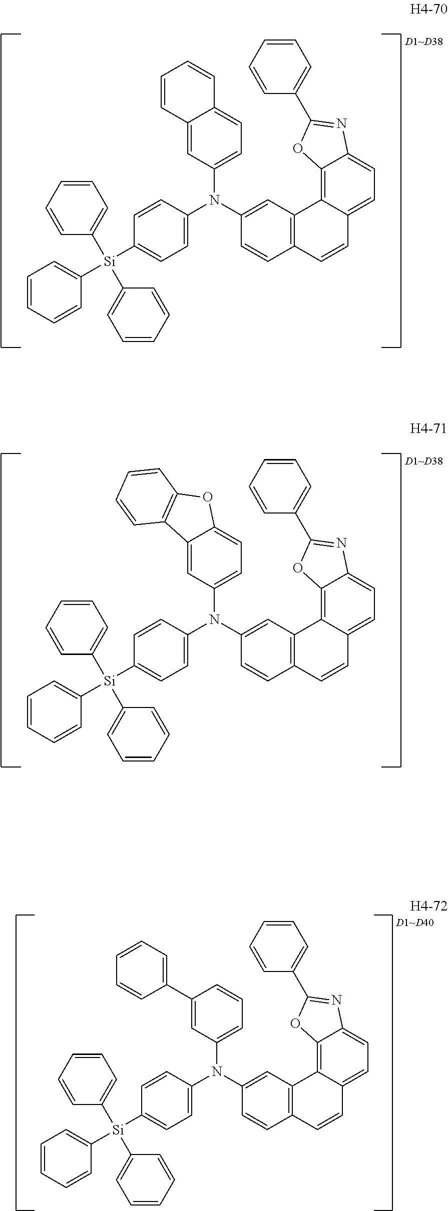 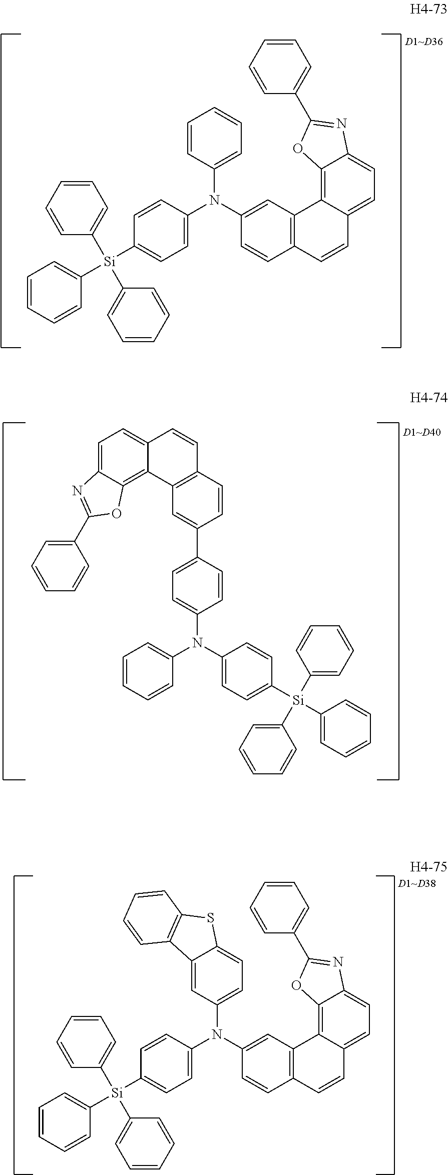 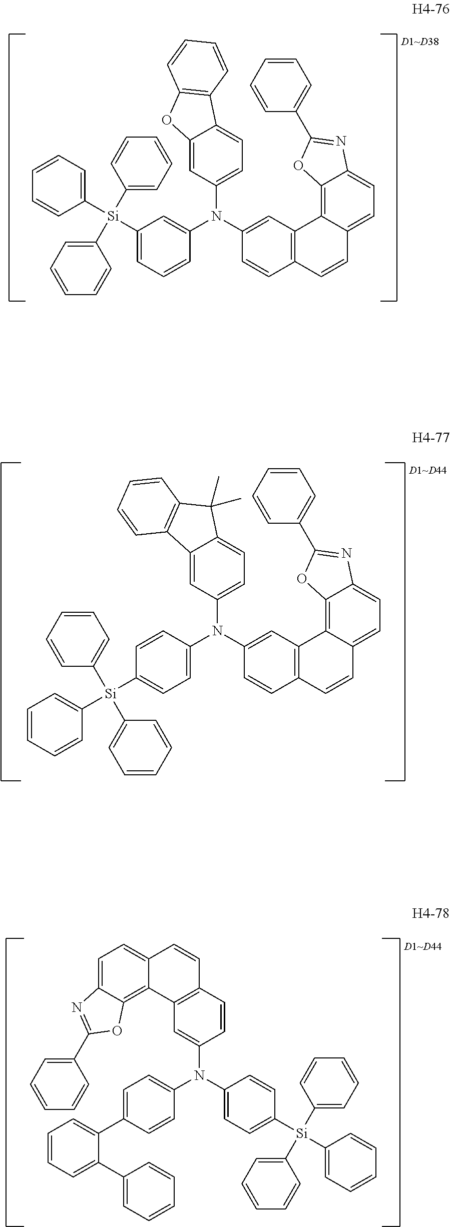 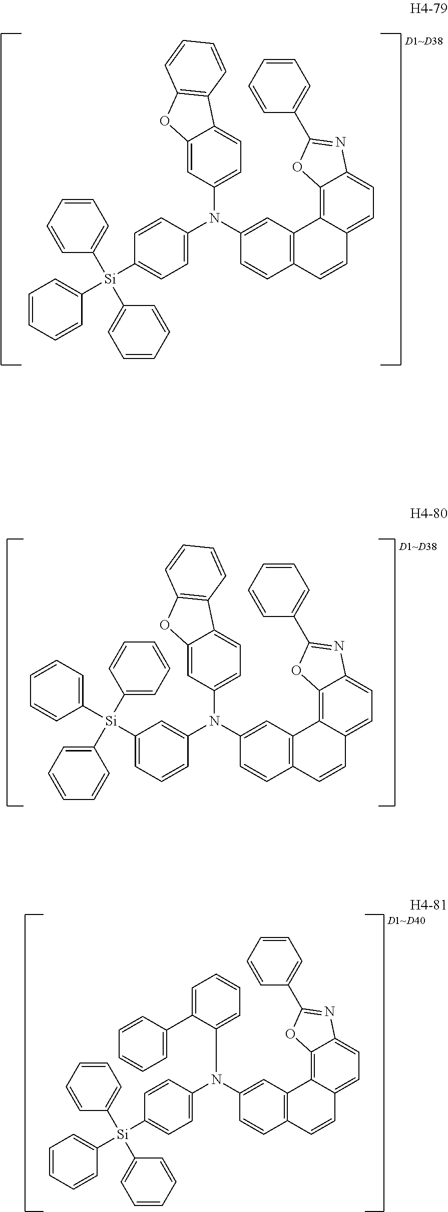 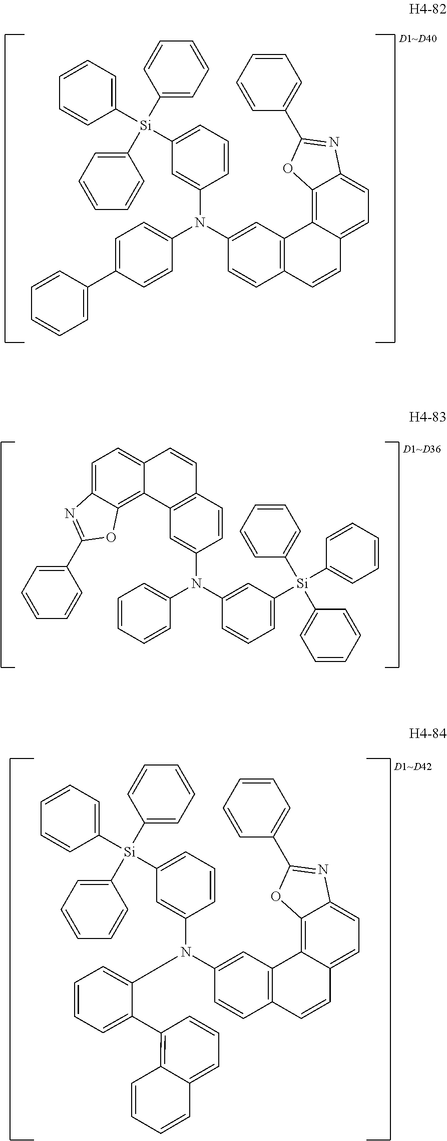 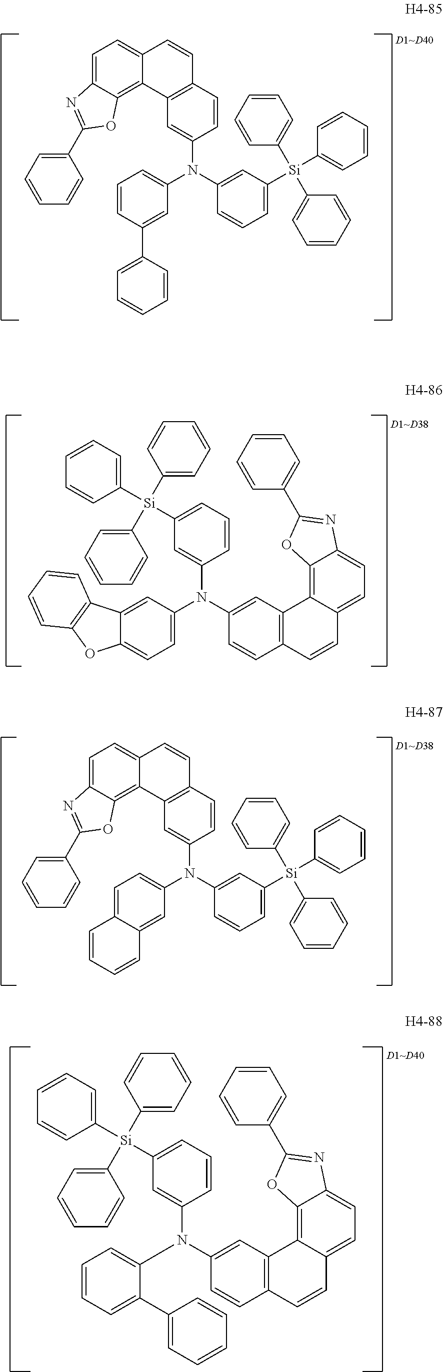 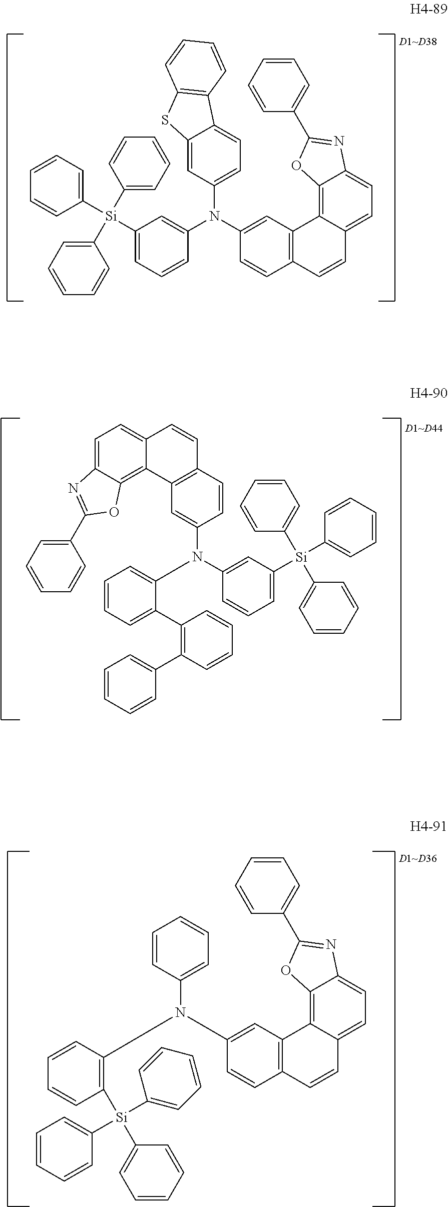 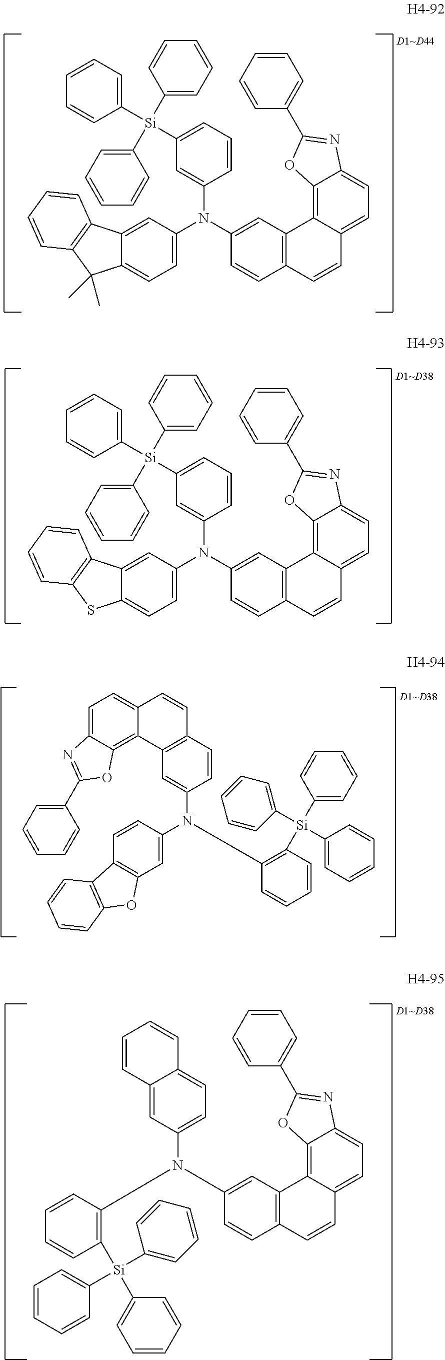 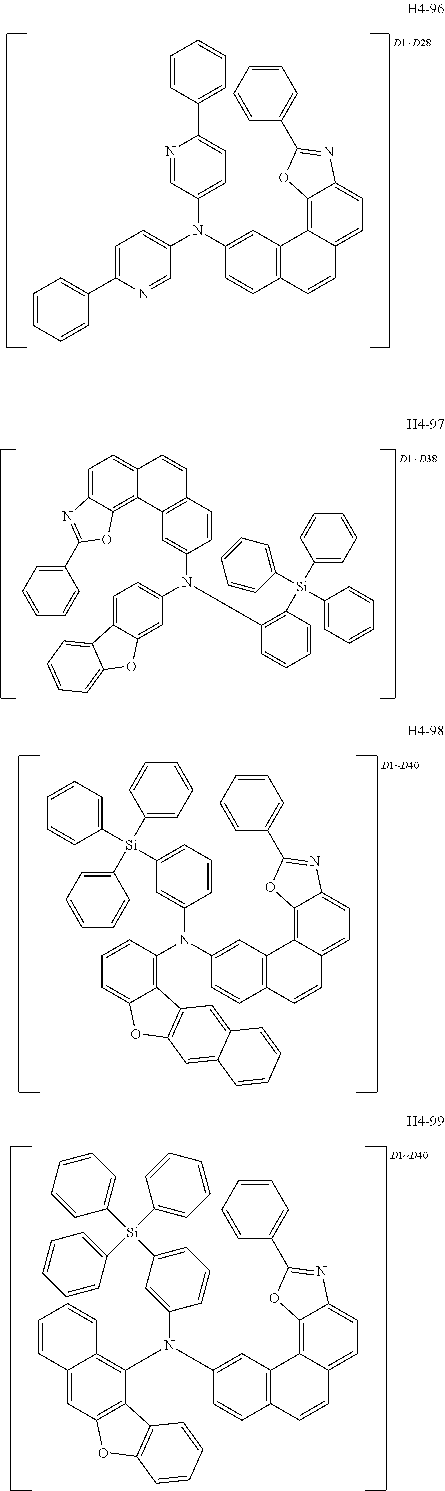 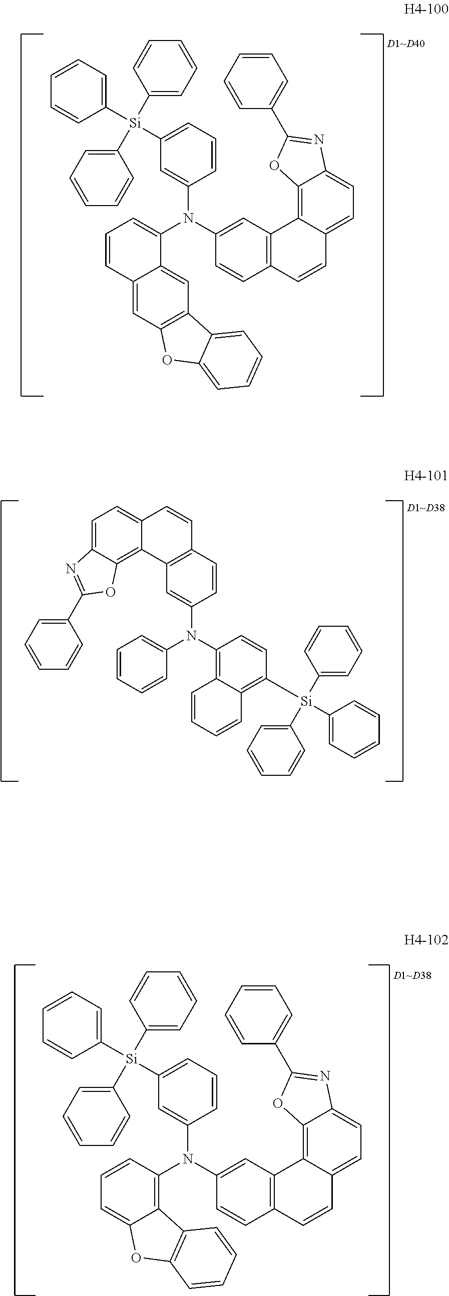 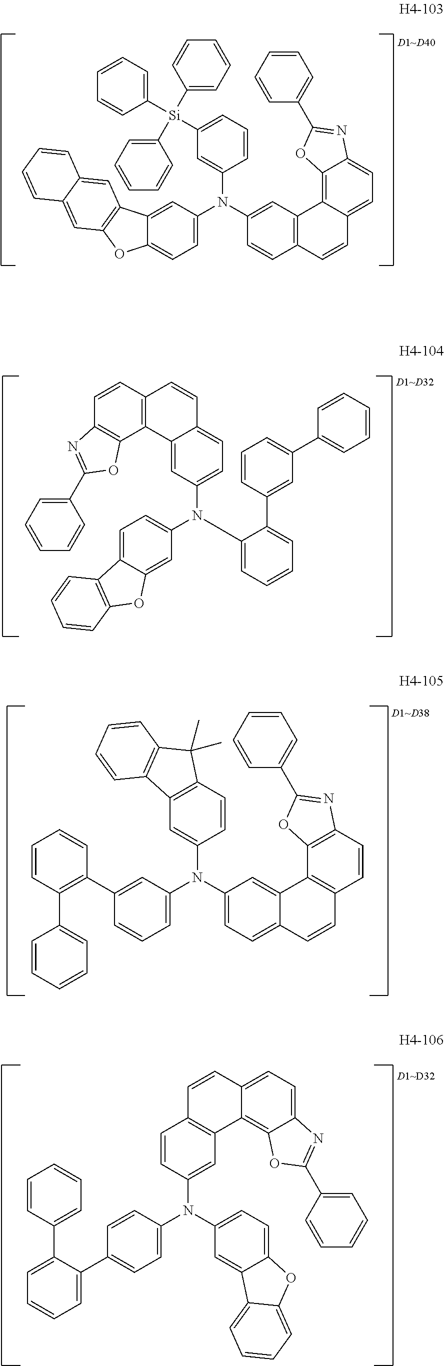 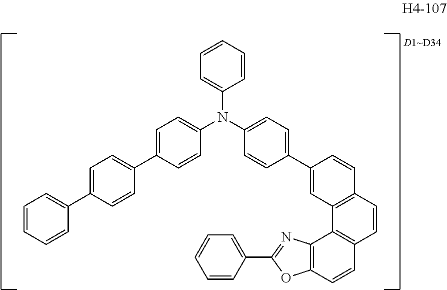 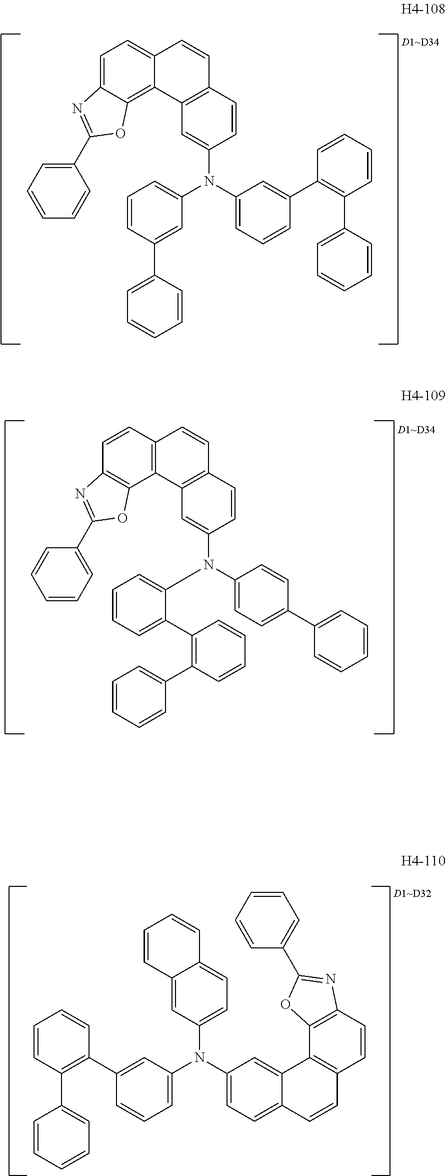 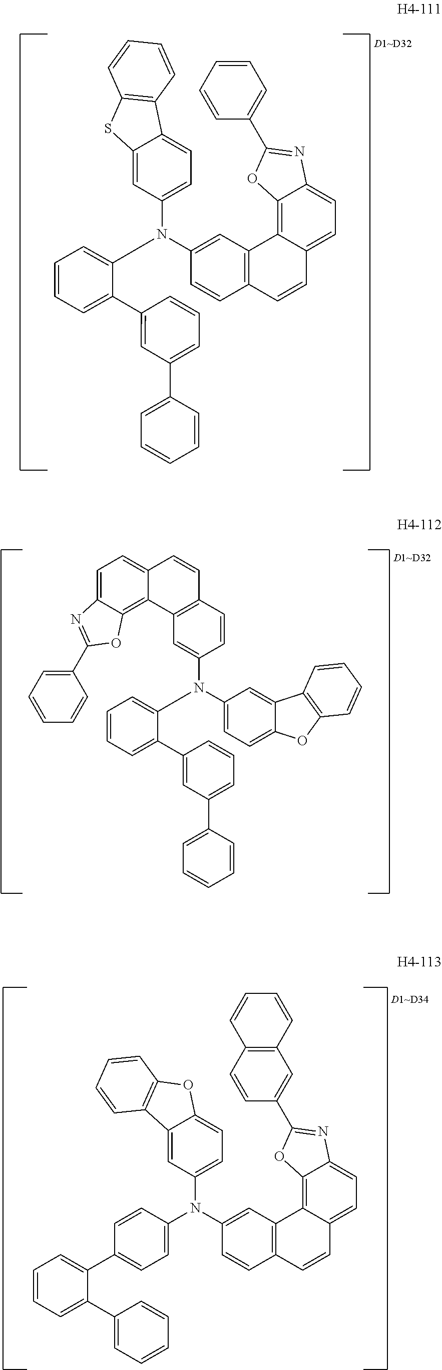 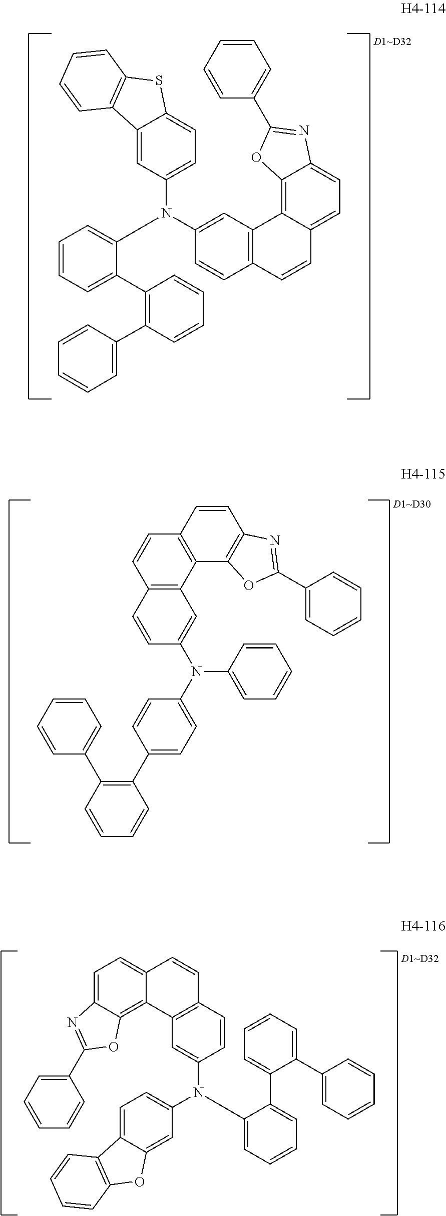 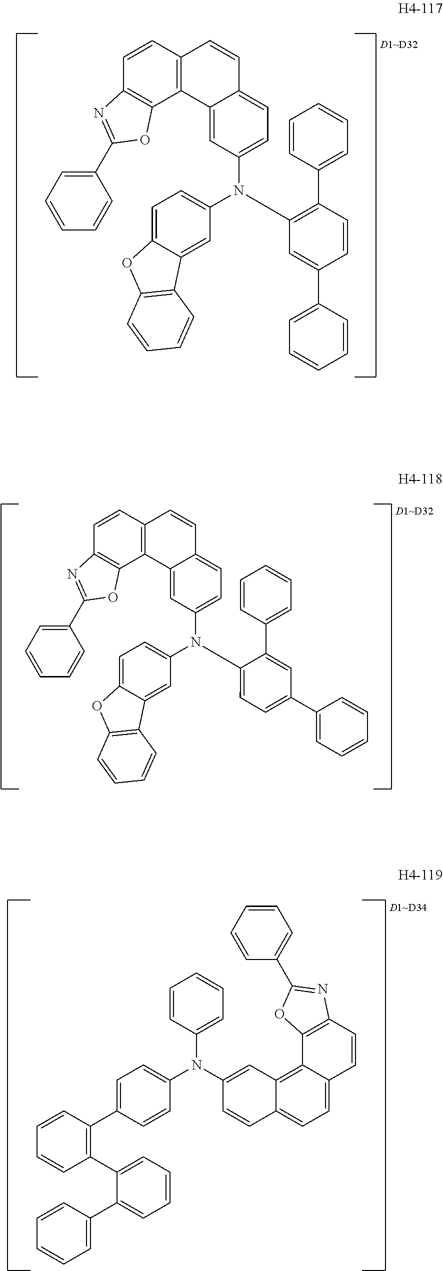 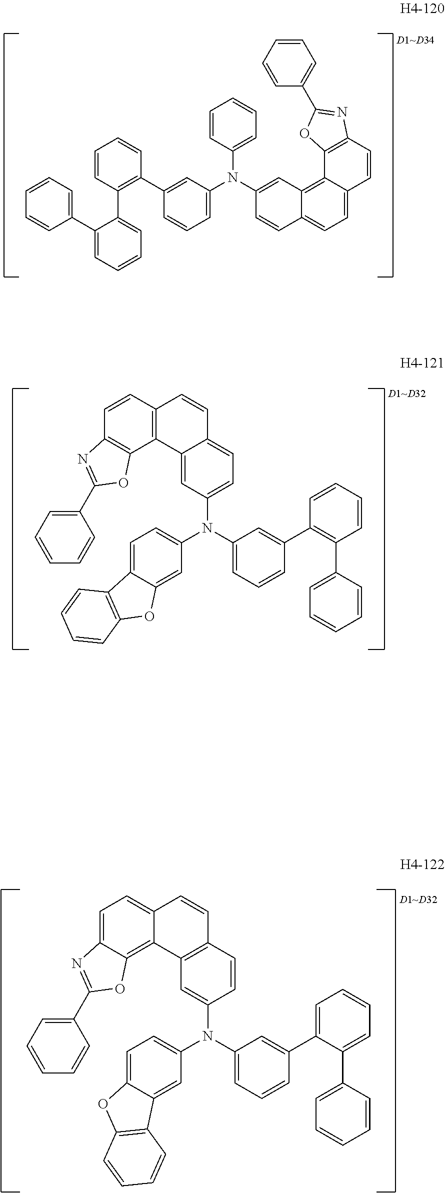 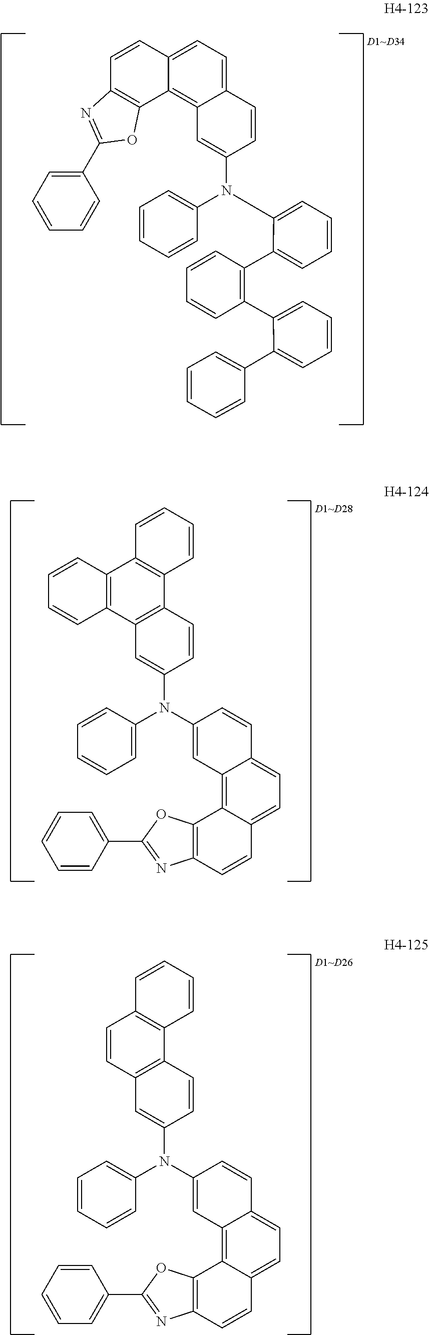 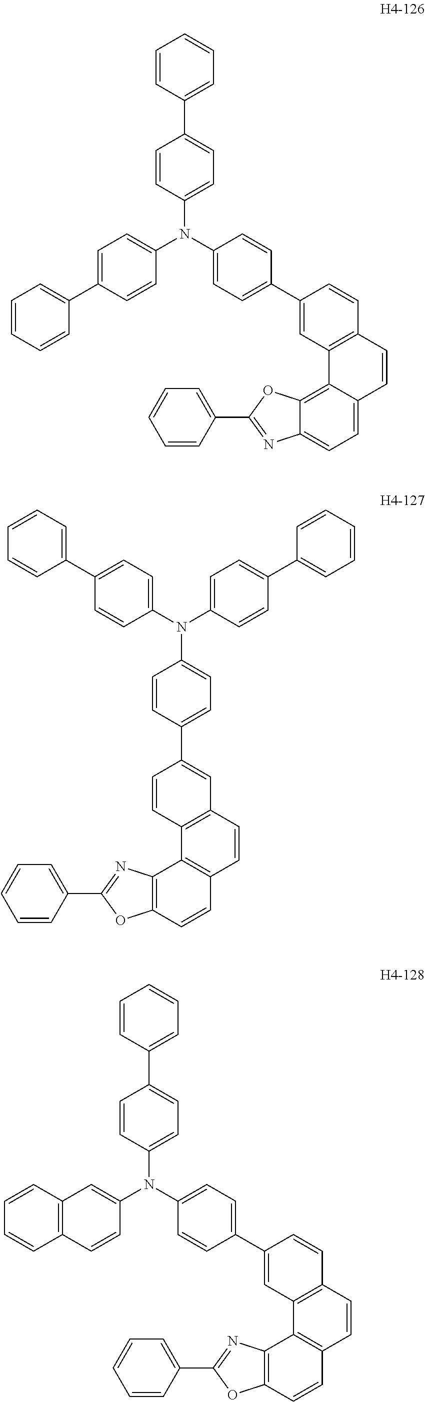 [SMILES: c1ccc(-c2ccc(N(c3ccc(-c4ccccc4)cc3)c3ccc(-c4ccc5ccc6ccc7nc(-c8ccccc8)oc7c6c5c4)cc3)cc2)cc1 ; c1ccc(-c2ccc(N(c3ccc(-c4ccccc4)cc3)c3ccc(-c4ccc5c(ccc6ccc7oc(-c8ccccc8)nc7c65)c4)cc3)cc2)cc1 ; c1ccc(-c2ccc(N(c3ccc(-c4ccc5ccc6ccc7oc(-c8ccccc8)nc7c6c5c4)cc3)c3ccc4ccccc4c3)cc2)cc1] 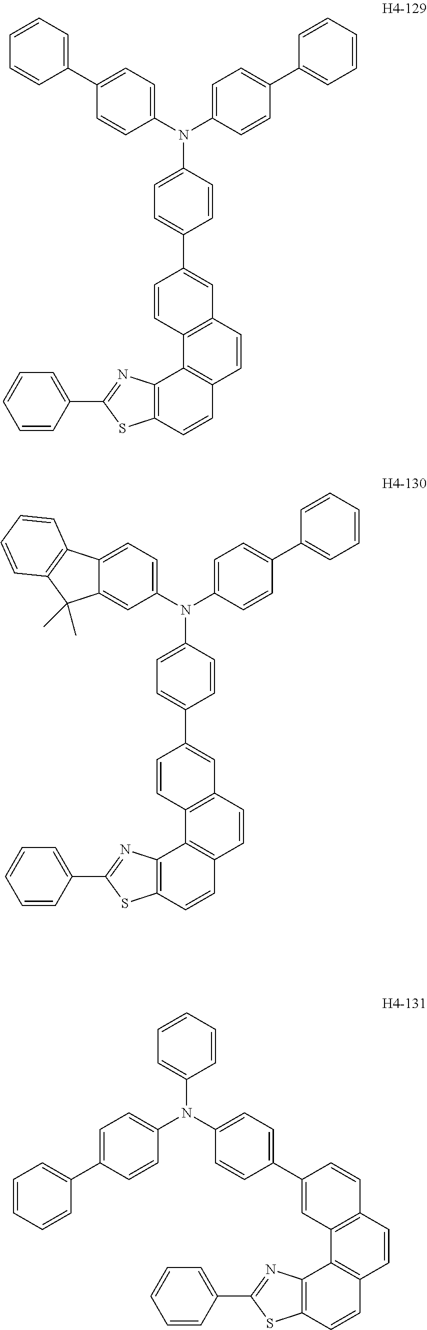 [SMILES: c1ccc(-c2ccc(N(c3ccc(-c4ccccc4)cc3)c3ccc(-c4ccc5c(ccc6ccc7sc(-c8ccccc8)nc7c65)c4)cc3)cc2)cc1 ; CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21 ; c1ccc(-c2ccc(N(c3ccccc3)c3ccc(-c4ccc5ccc6ccc7sc(-c8ccccc8)nc7c6c5c4)cc3)cc2)cc1] 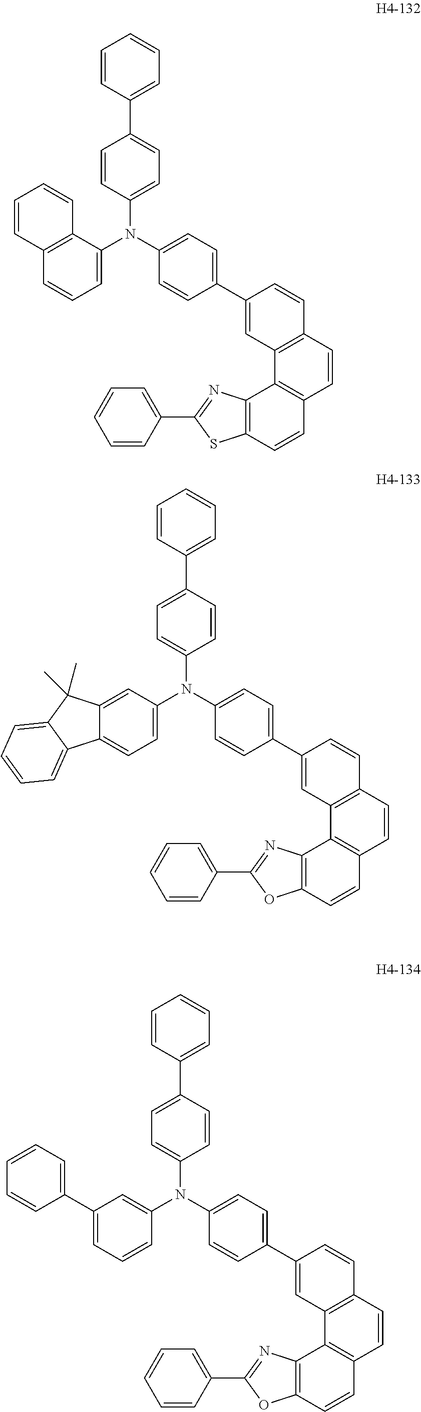 [SMILES: c1ccc(-c2ccc(N(c3ccc(-c4ccc5ccc6ccc7sc(-c8ccccc8)nc7c6c5c4)cc3)c3cccc4ccccc34)cc2)cc1 ; CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21 ; c1ccc(-c2ccc(N(c3ccc(-c4ccc5ccc6ccc7oc(-c8ccccc8)nc7c6c5c4)cc3)c3cccc(-c4ccccc4)c3)cc2)cc1] 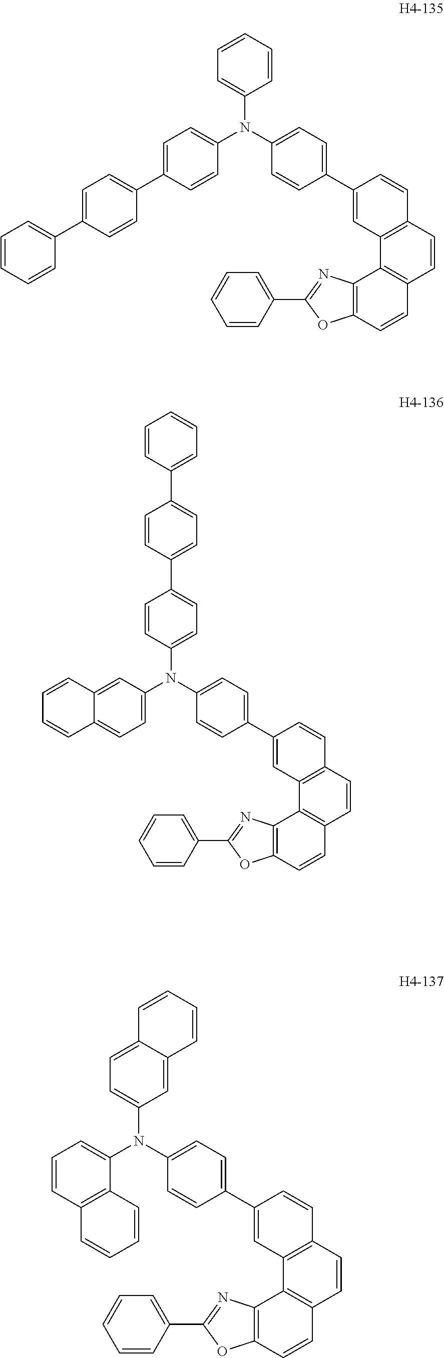 [SMILES: c1ccc(-c2ccc(-c3ccc(N(c4ccccc4)c4ccc(-c5ccc6ccc7ccc8oc(-c9ccccc9)nc8c7c6c5)cc4)cc3)cc2)cc1 ; c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1 ; c1ccc(-c2nc3c(ccc4ccc5ccc(-c6ccc(N(c7ccc8ccccc8c7)c7cccc8ccccc78)cc6)cc5c43)o2)cc1] 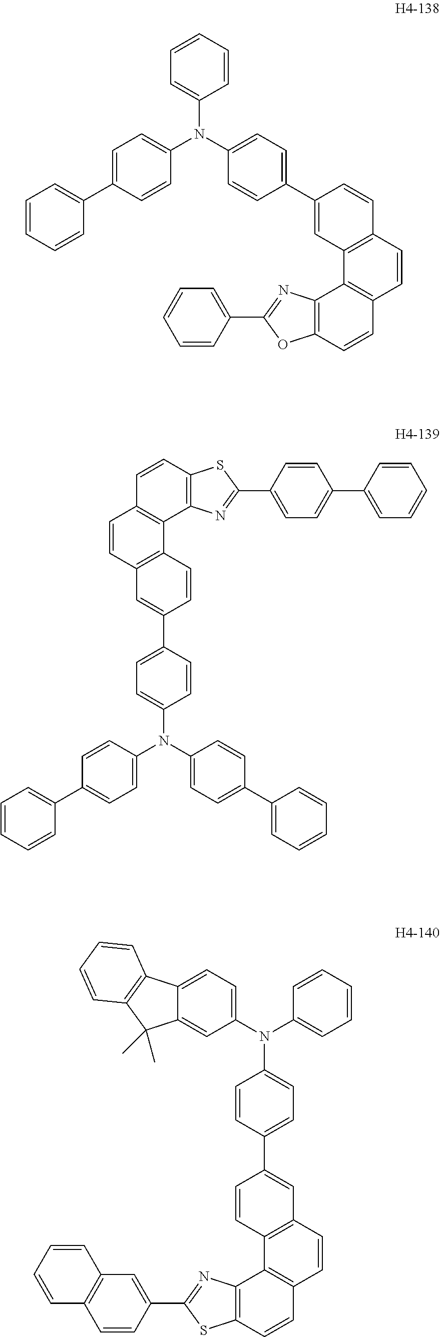 [SMILES: c1ccc(-c2ccc(N(c3ccccc3)c3ccc(-c4ccc5ccc6ccc7oc(-c8ccccc8)nc7c6c5c4)cc3)cc2)cc1 ; c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1 ; CC1(C)c2ccccc2-c2ccc(N(c3ccccc3)c3ccc(-c4ccc5c(ccc6ccc7sc(-c8ccc9ccccc9c8)nc7c65)c4)cc3)cc21] 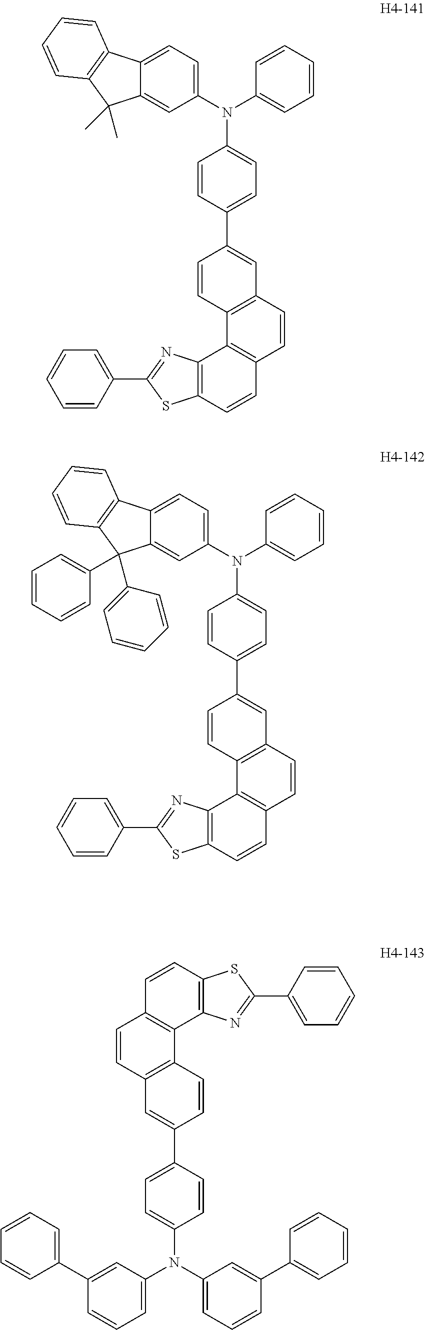 [SMILES: CC1(C)c2ccccc2-c2ccc(N(c3ccccc3)c3ccc(-c4ccc5c(ccc6ccc7sc(-c8ccccc8)nc7c65)c4)cc3)cc21 ; c1ccc(-c2nc3c(ccc4ccc5cc(-c6ccc(N(c7ccccc7)c7ccc8c(c7)C(c7ccccc7)(c7ccccc7)c7ccccc7-8)cc6)ccc5c43)s2)cc1 ; c1ccc(-c2cccc(N(c3ccc(-c4ccc5c(ccc6ccc7sc(-c8ccccc8)nc7c65)c4)cc3)c3cccc(-c4ccccc4)c3)c2)cc1] 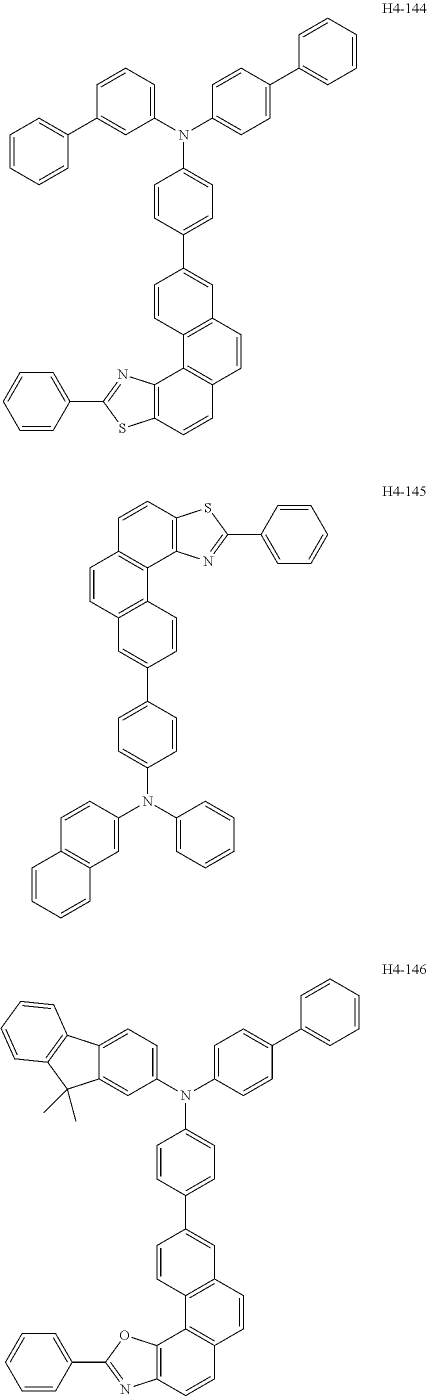 [SMILES: c1ccc(-c2ccc(N(c3ccc(-c4ccc5c(ccc6ccc7sc(-c8ccccc8)nc7c65)c4)cc3)c3cccc(-c4ccccc4)c3)cc2)cc1 ; c1ccc(-c2nc3c(ccc4ccc5cc(-c6ccc(N(c7ccccc7)c7ccc8ccccc8c7)cc6)ccc5c43)s2)cc1 ; CC1(C)c2ccccc2-c2ccc(N(c3ccc(-c4ccccc4)cc3)c3ccc(-c4ccc5c(ccc6ccc7nc(-c8ccccc8)oc7c65)c4)cc3)cc21] 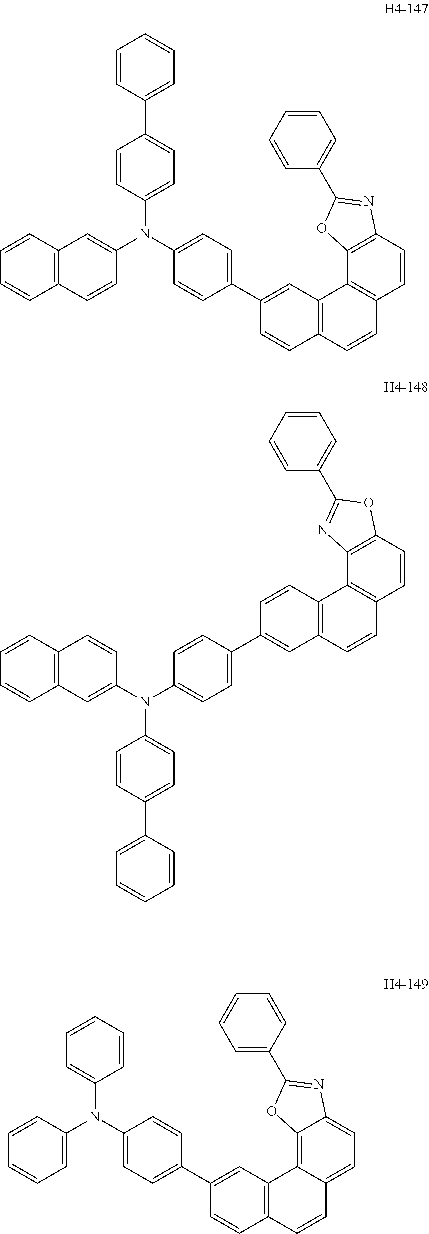 [SMILES: c1ccc(-c2ccc(N(c3ccc(-c4ccc5ccc6ccc7nc(-c8ccccc8)oc7c6c5c4)cc3)c3ccc4ccccc4c3)cc2)cc1 ; c1ccc(-c2ccc(N(c3ccc(-c4ccc5c(ccc6ccc7oc(-c8ccccc8)nc7c65)c4)cc3)c3ccc4ccccc4c3)cc2)cc1 ; c1ccc(-c2nc3ccc4ccc5ccc(-c6ccc(N(c7ccccc7)c7ccccc7)cc6)cc5c4c3o2)cc1] 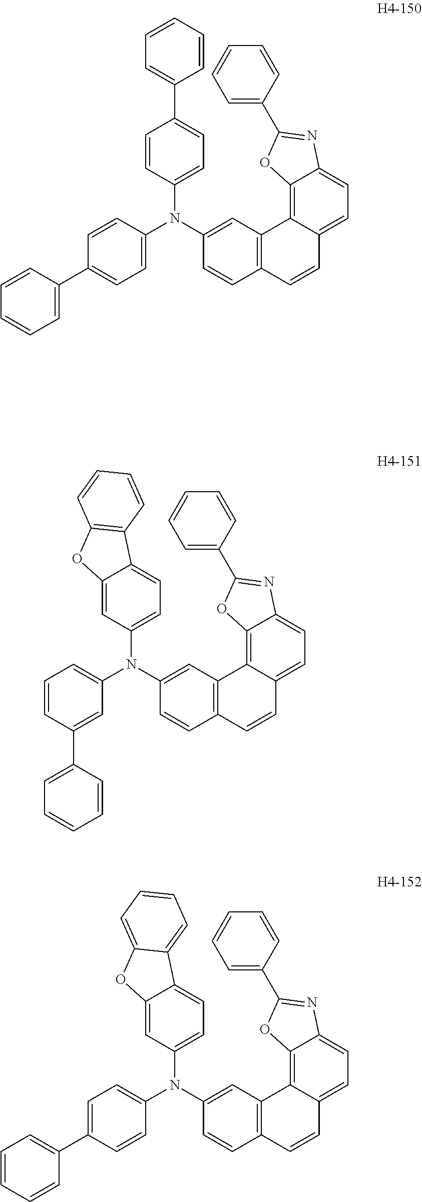 [SMILES: c1ccc(-c2ccc(N(c3ccc(-c4ccccc4)cc3)c3ccc4ccc5ccc6nc(-c7ccccc7)oc6c5c4c3)cc2)cc1 ; c1ccc(-c2cccc(N(c3ccc4c(c3)oc3ccccc34)c3ccc4ccc5ccc6nc(-c7ccccc7)oc6c5c4c3)c2)cc1 ; c1ccc(-c2ccc(N(c3ccc4c(c3)oc3ccccc34)c3ccc4ccc5ccc6nc(-c7ccccc7)oc6c5c4c3)cc2)cc1] 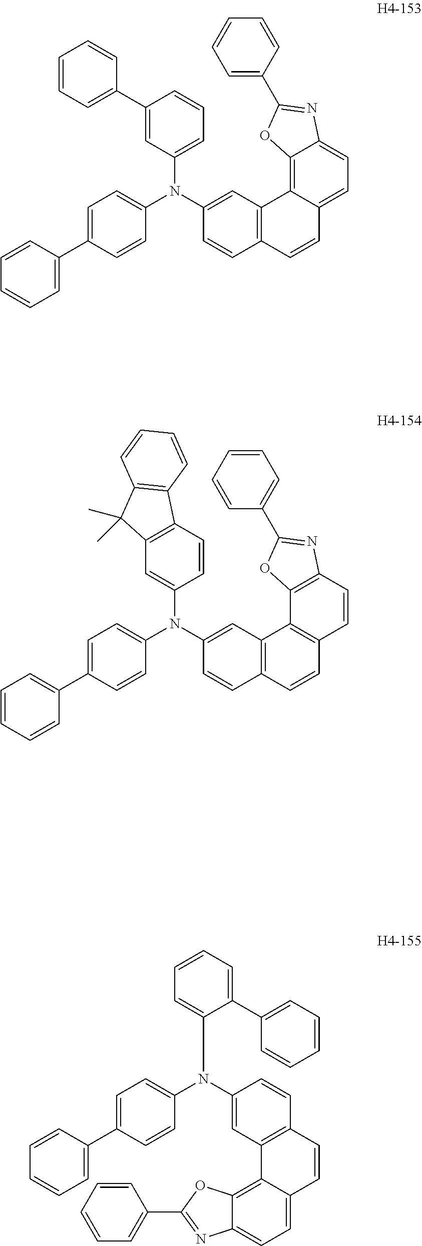 [SMILES: c1ccc(-c2ccc(N(c3cccc(-c4ccccc4)c3)c3ccc4ccc5ccc6nc(-c7ccccc7)oc6c5c4c3)cc2)cc1 ; CC1(C)c2ccccc2-c2ccc(N(c3ccc(-c4ccccc4)cc3)c3ccc4ccc5ccc6nc(-c7ccccc7)oc6c5c4c3)cc21 ; c1ccc(-c2ccc(N(c3ccc4ccc5ccc6nc(-c7ccccc7)oc6c5c4c3)c3ccccc3-c3ccccc3)cc2)cc1] 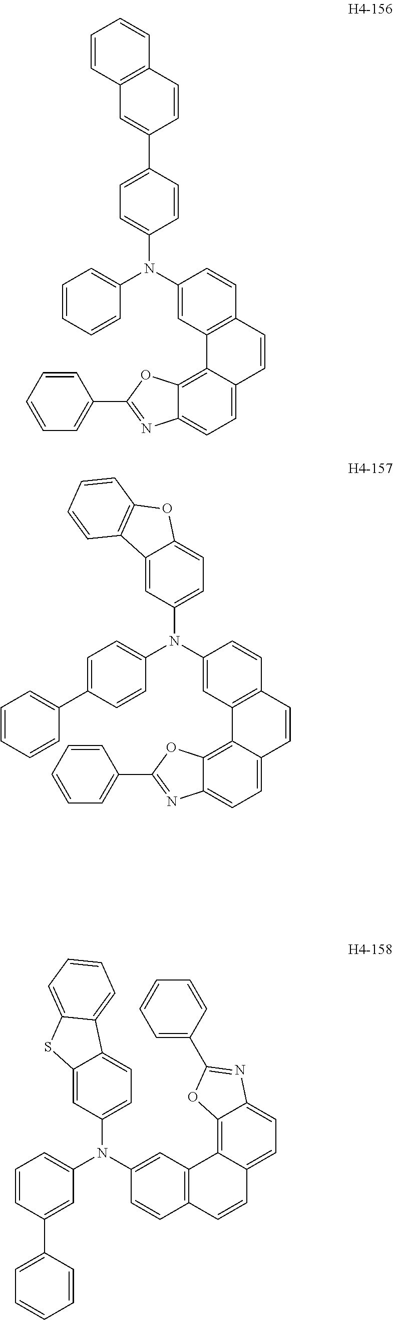 [SMILES: c1ccc(-c2nc3ccc4ccc5ccc(N(c6ccccc6)c6ccc(-c7ccc8ccccc8c7)cc6)cc5c4c3o2)cc1 ; c1ccc(-c2ccc(N(c3ccc4oc5ccccc5c4c3)c3ccc4ccc5ccc6nc(-c7ccccc7)oc6c5c4c3)cc2)cc1 ; c1ccc(-c2cccc(N(c3ccc4c(c3)sc3ccccc34)c3ccc4ccc5ccc6nc(-c7ccccc7)oc6c5c4c3)c2)cc1] 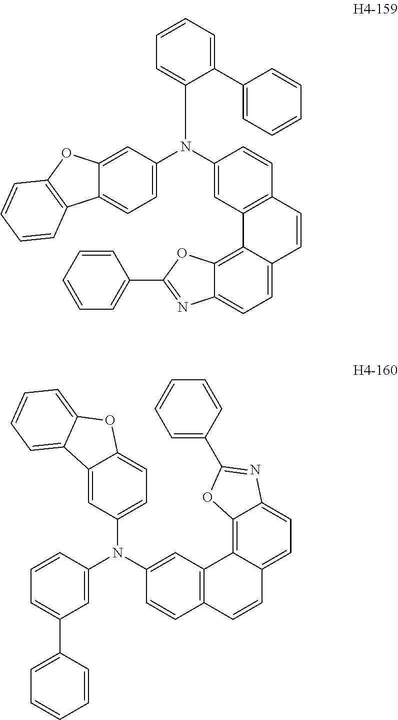 [SMILES: c1ccc(-c2nc3ccc4ccc5ccc(N(c6ccc7c(c6)oc6ccccc67)c6ccccc6-c6ccccc6)cc5c4c3o2)cc1 ; c1ccc(-c2cccc(N(c3ccc4oc5ccccc5c4c3)c3ccc4ccc5ccc6nc(-c7ccccc7)oc6c5c4c3)c2)cc1] 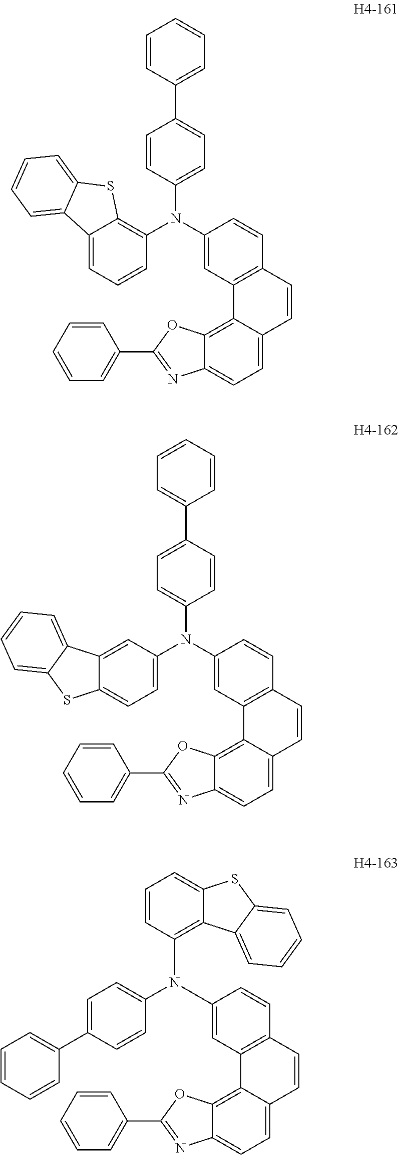 [SMILES: c1ccc(-c2ccc(N(c3ccc4ccc5ccc6nc(-c7ccccc7)oc6c5c4c3)c3cccc4c3sc3ccccc34)cc2)cc1 ; c1ccc(-c2ccc(N(c3ccc4sc5ccccc5c4c3)c3ccc4ccc5ccc6nc(-c7ccccc7)oc6c5c4c3)cc2)cc1 ; c1ccc(-c2ccc(N(c3ccc4ccc5ccc6nc(-c7ccccc7)oc6c5c4c3)c3cccc4sc5ccccc5c34)cc2)cc1] 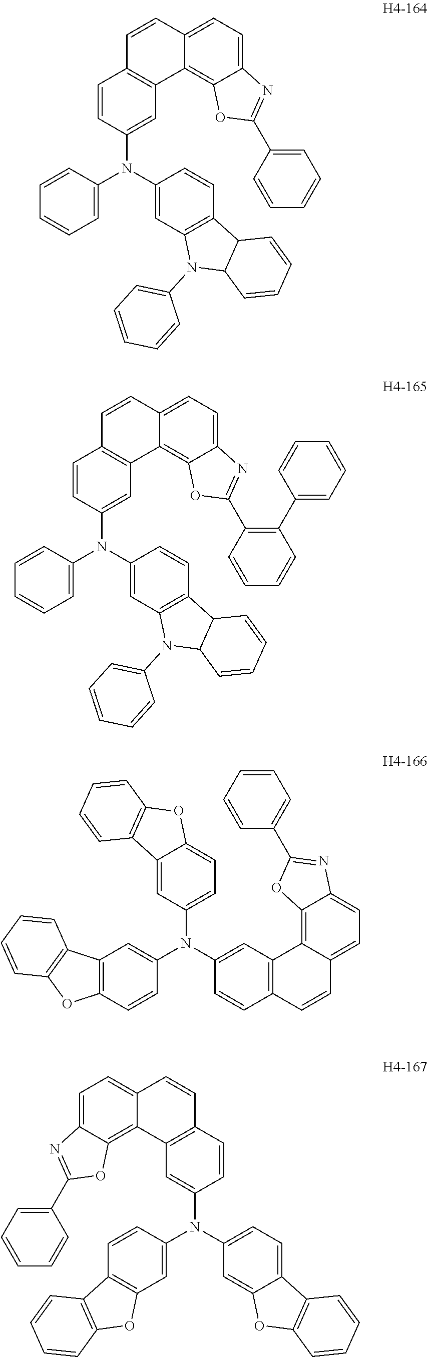 [SMILES: C1=CC2c3ccc(N(c4ccccc4)c4ccc5ccc6ccc7nc(-c8ccccc8)oc7c6c5c4)cc3N(c3ccccc3)C2C=C1 ; C1=CC2c3ccc(N(c4ccccc4)c4ccc5ccc6ccc7nc(-c8ccccc8-c8ccccc8)oc7c6c5c4)cc3N(c3ccccc3)C2C=C1 ; c1ccc(-c2nc3ccc4ccc5ccc(N(c6ccc7oc8ccccc8c7c6)c6ccc7oc8ccccc8c7c6)cc5c4c3o2)cc1 ; c1ccc(-c2nc3ccc4ccc5ccc(N(c6ccc7c(c6)oc6ccccc67)c6ccc7c(c6)oc6ccccc67)cc5c4c3o2)cc1] 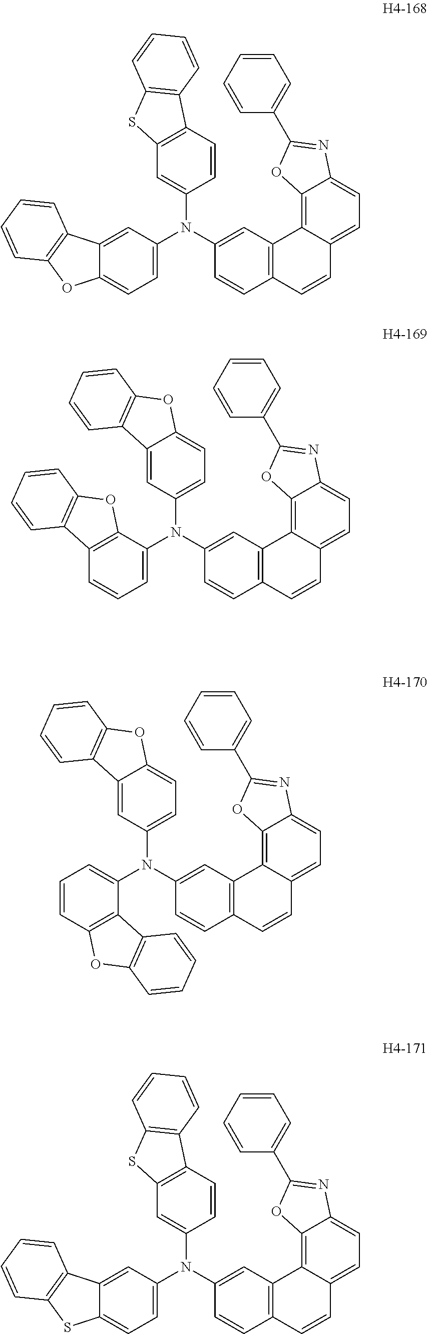 [SMILES: c1ccc(-c2nc3ccc4ccc5ccc(N(c6ccc7c(c6)sc6ccccc67)c6ccc7oc8ccccc8c7c6)cc5c4c3o2)cc1 ; c1ccc(-c2nc3ccc4ccc5ccc(N(c6ccc7oc8ccccc8c7c6)c6cccc7c6oc6ccccc67)cc5c4c3o2)cc1 ; c1ccc(-c2nc3ccc4ccc5ccc(N(c6ccc7oc8ccccc8c7c6)c6cccc7oc8ccccc8c67)cc5c4c3o2)cc1 ; c1ccc(-c2nc3ccc4ccc5ccc(N(c6ccc7c(c6)sc6ccccc67)c6ccc7sc8ccccc8c7c6)cc5c4c3o2)cc1] 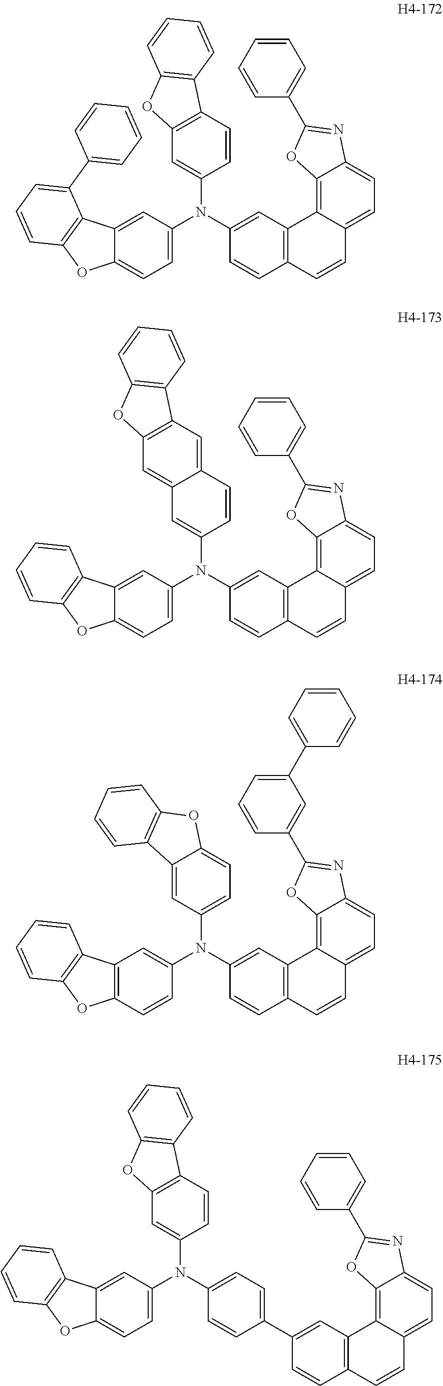 [SMILES: c1ccc(-c2nc3ccc4ccc5ccc(N(c6ccc7c(c6)oc6ccccc67)c6ccc7oc8cccc(-c9ccccc9)c8c7c6)cc5c4c3o2)cc1 ; c1ccc(-c2nc3ccc4ccc5ccc(N(c6ccc7cc8c(cc7c6)oc6ccccc68)c6ccc7oc8ccccc8c7c6)cc5c4c3o2)cc1 ; c1ccc(-c2cccc(-c3nc4ccc5ccc6ccc(N(c7ccc8oc9ccccc9c8c7)c7ccc8oc9ccccc9c8c7)cc6c5c4o3)c2)cc1 ; c1ccc(-c2nc3ccc4ccc5ccc(-c6ccc(N(c7ccc8c(c7)oc7ccccc78)c7ccc8oc9ccccc9c8c7)cc6)cc5c4c3o2)cc1] 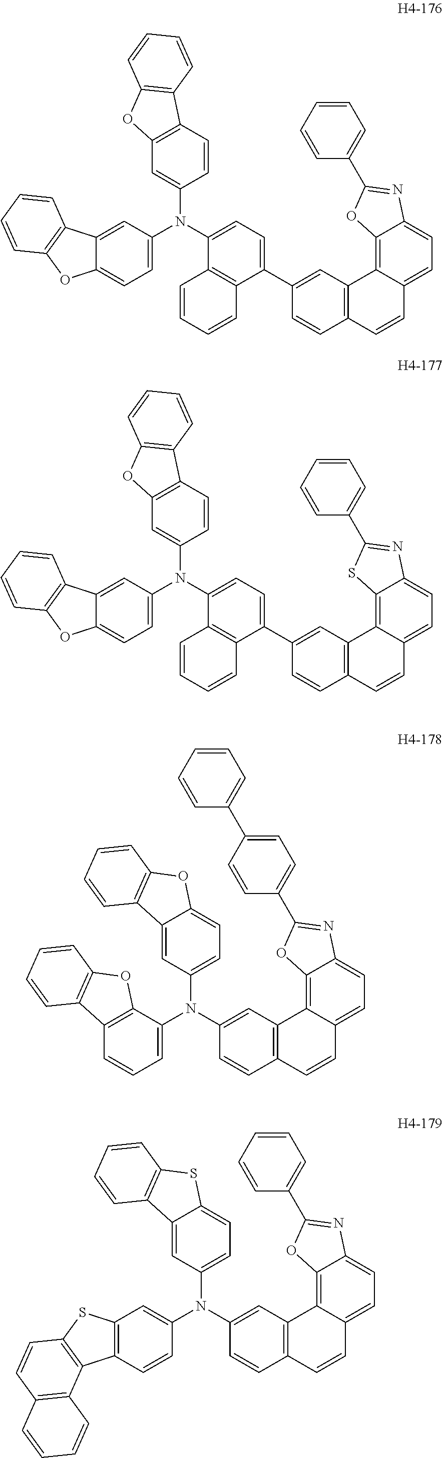 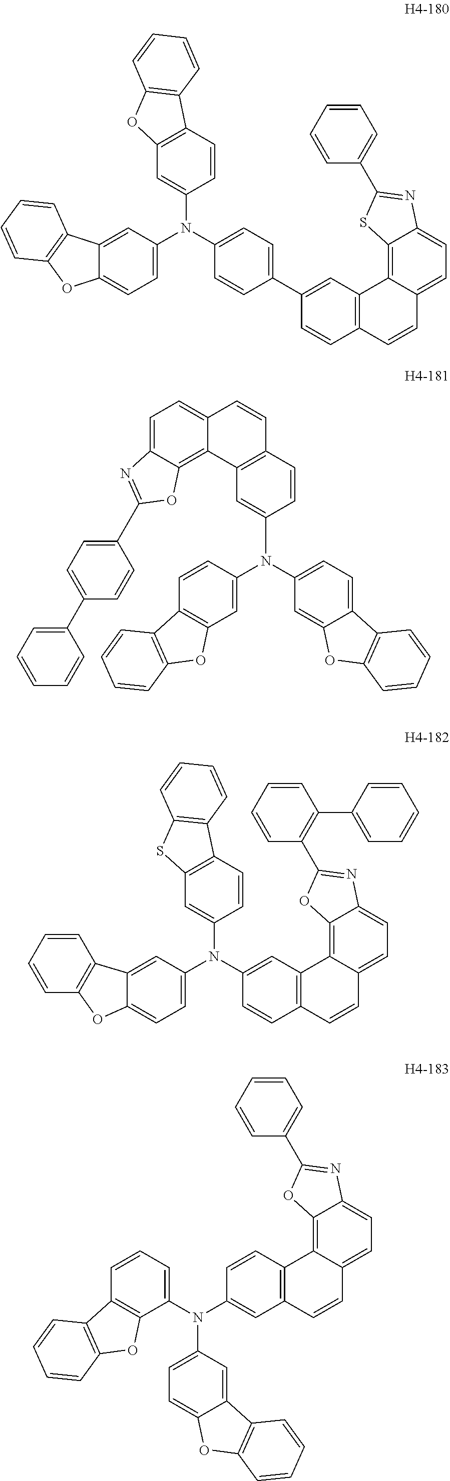 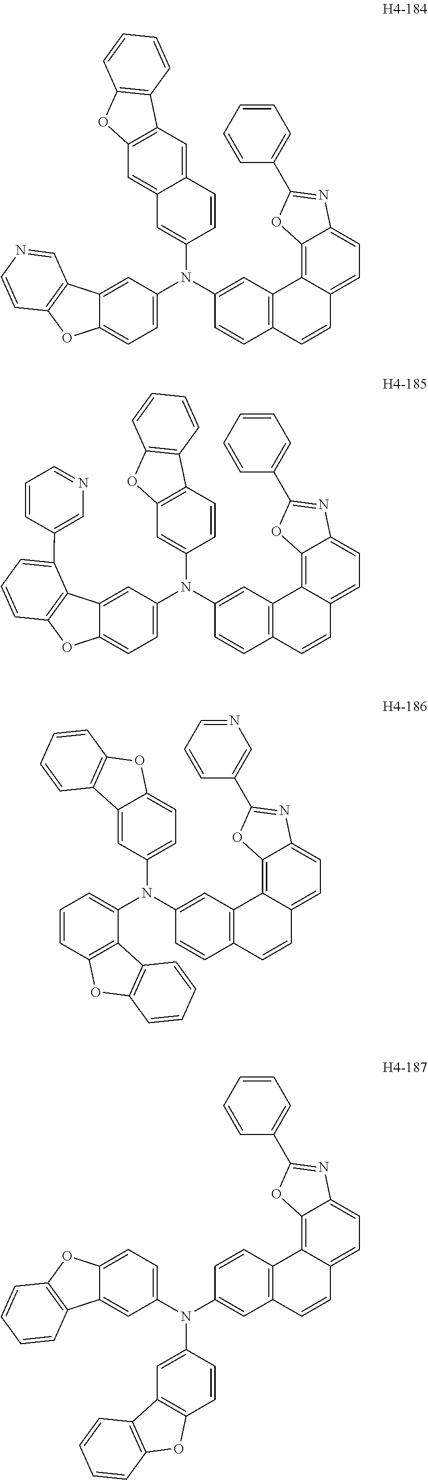 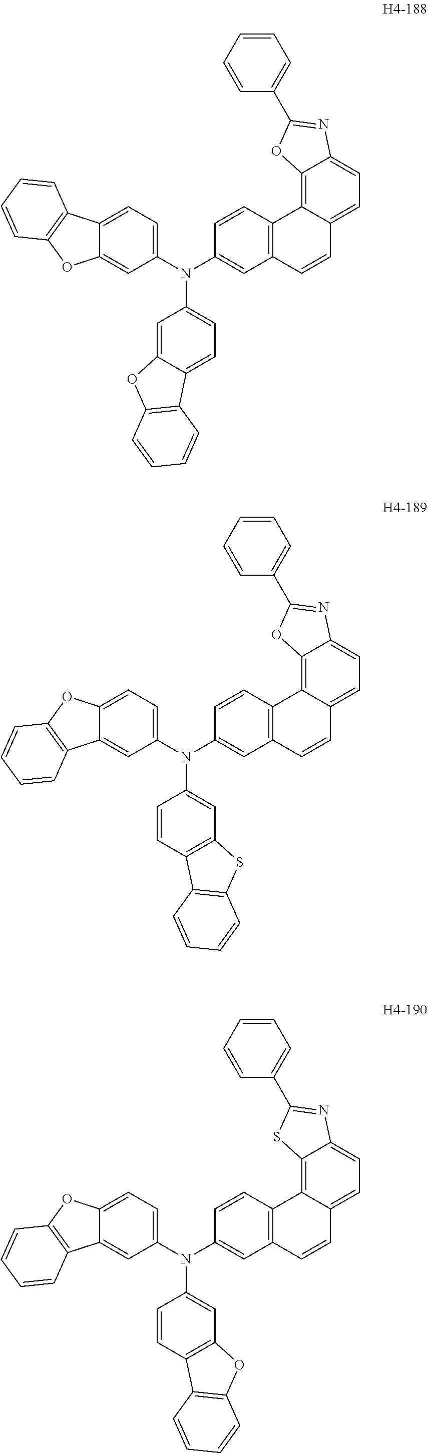 [SMILES: c1ccc(-c2nc3ccc4ccc5cc(N(c6ccc7c(c6)oc6ccccc67)c6ccc7c(c6)oc6ccccc67)ccc5c4c3o2)cc1 ; c1ccc(-c2nc3ccc4ccc5cc(N(c6ccc7c(c6)sc6ccccc67)c6ccc7oc8ccccc8c7c6)ccc5c4c3o2)cc1 ; c1ccc(-c2nc3ccc4ccc5cc(N(c6ccc7c(c6)oc6ccccc67)c6ccc7oc8ccccc8c7c6)ccc5c4c3s2)cc1] 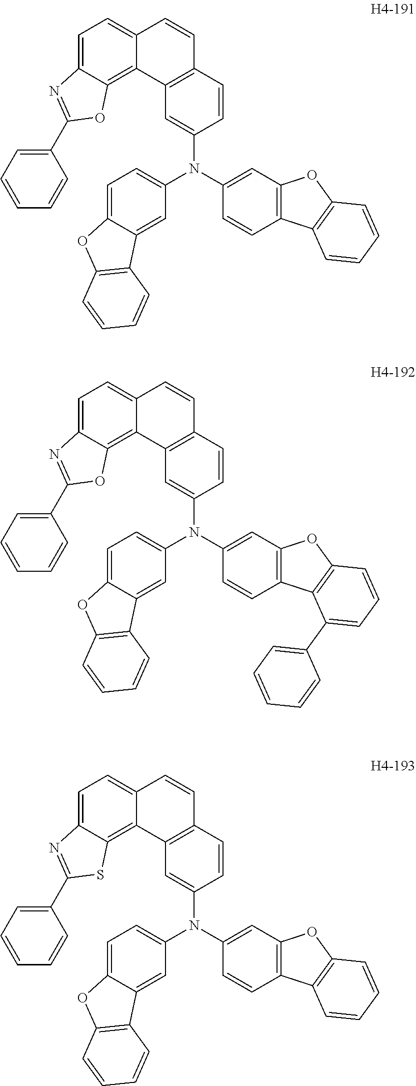 [SMILES: c1ccc(-c2nc3ccc4ccc5ccc(N(c6ccc7c(c6)oc6ccccc67)c6ccc7oc8ccccc8c7c6)cc5c4c3o2)cc1 ; c1ccc(-c2nc3ccc4ccc5ccc(N(c6ccc7c(c6)oc6cccc(-c8ccccc8)c67)c6ccc7oc8ccccc8c7c6)cc5c4c3o2)cc1 ; c1ccc(-c2nc3ccc4ccc5ccc(N(c6ccc7c(c6)oc6ccccc67)c6ccc7oc8ccccc8c7c6)cc5c4c3s2)cc1] 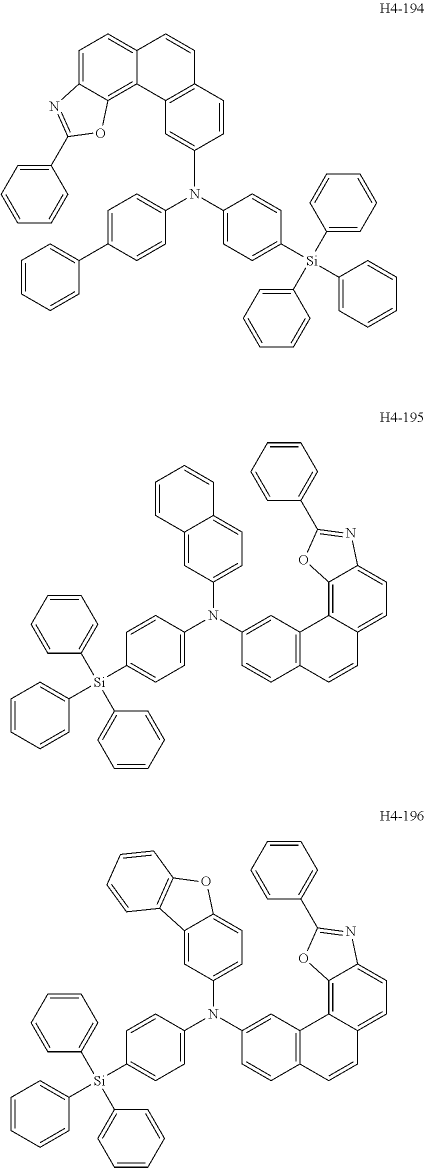 [SMILES: c1ccc(-c2ccc(N(c3ccc([Si](c4ccccc4)(c4ccccc4)c4ccccc4)cc3)c3ccc4ccc5ccc6nc(-c7ccccc7)oc6c5c4c3)cc2)cc1 ; c1ccc(-c2nc3ccc4ccc5ccc(N(c6ccc([Si](c7ccccc7)(c7ccccc7)c7ccccc7)cc6)c6ccc7ccccc7c6)cc5c4c3o2)cc1 ; c1ccc(-c2nc3ccc4ccc5ccc(N(c6ccc([Si](c7ccccc7)(c7ccccc7)c7ccccc7)cc6)c6ccc7oc8ccccc8c7c6)cc5c4c3o2)cc1] 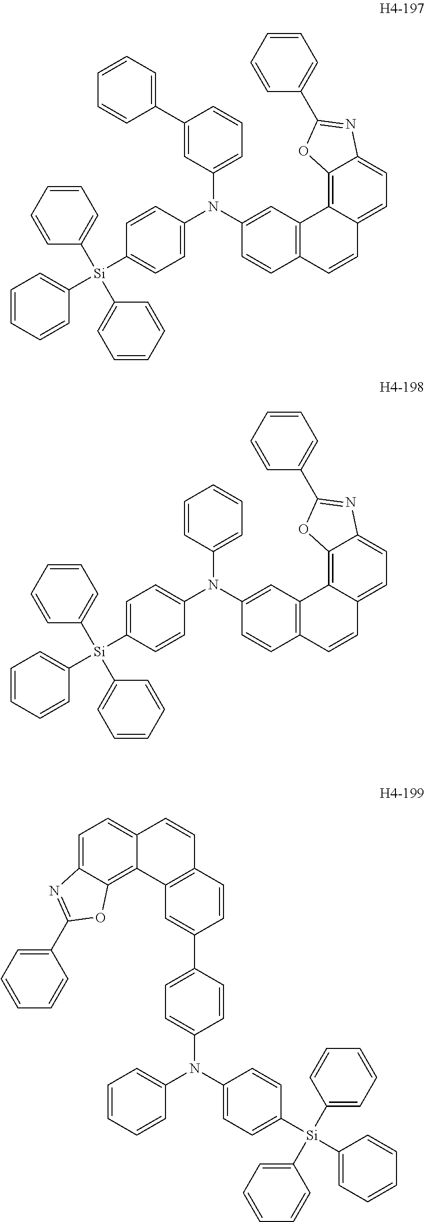 [SMILES: c1ccc(-c2cccc(N(c3ccc([Si](c4ccccc4)(c4ccccc4)c4ccccc4)cc3)c3ccc4ccc5ccc6nc(-c7ccccc7)oc6c5c4c3)c2)cc1 ; c1ccc(-c2nc3ccc4ccc5ccc(N(c6ccccc6)c6ccc([Si](c7ccccc7)(c7ccccc7)c7ccccc7)cc6)cc5c4c3o2)cc1 ; c1ccc(-c2nc3ccc4ccc5ccc(-c6ccc(N(c7ccccc7)c7ccc([Si](c8ccccc8)(c8ccccc8)c8ccccc8)cc7)cc6)cc5c4c3o2)cc1] 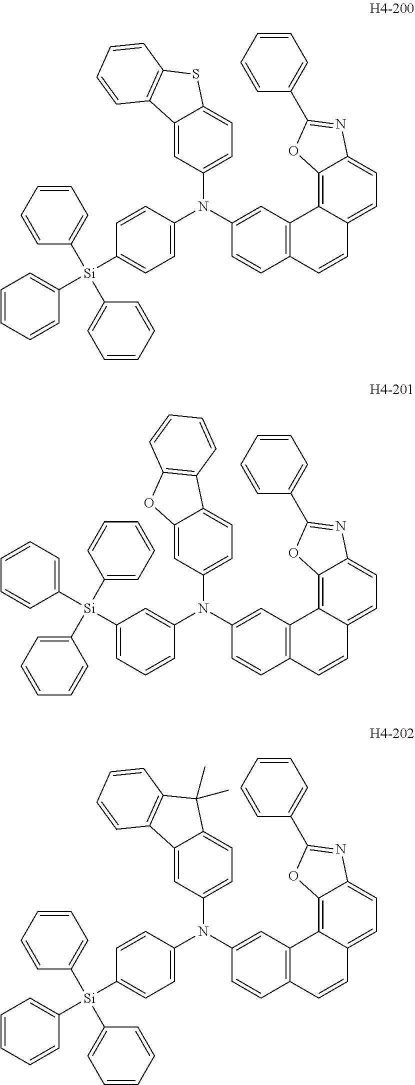 [SMILES: c1ccc(-c2nc3ccc4ccc5ccc(N(c6ccc([Si](c7ccccc7)(c7ccccc7)c7ccccc7)cc6)c6ccc7sc8ccccc8c7c6)cc5c4c3o2)cc1 ; c1ccc(-c2nc3ccc4ccc5ccc(N(c6cccc([Si](c7ccccc7)(c7ccccc7)c7ccccc7)c6)c6ccc7c(c6)oc6ccccc67)cc5c4c3o2)cc1 ; CC1(C)c2ccccc2-c2cc(N(c3ccc([Si](c4ccccc4)(c4ccccc4)c4ccccc4)cc3)c3ccc4ccc5ccc6nc(-c7ccccc7)oc6c5c4c3)ccc21] 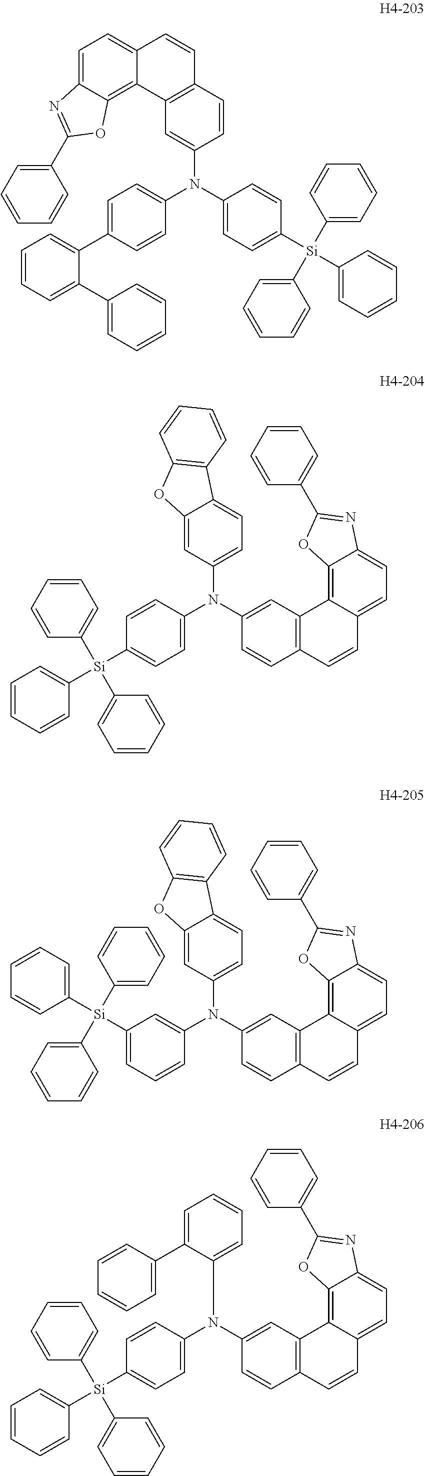 [SMILES: c1ccc(-c2nc3ccc4ccc5ccc(N(c6ccc(-c7ccccc7-c7ccccc7)cc6)c6ccc([Si](c7ccccc7)(c7ccccc7)c7ccccc7)cc6)cc5c4c3o2)cc1 ; c1ccc(-c2nc3ccc4ccc5ccc(N(c6ccc([Si](c7ccccc7)(c7ccccc7)c7ccccc7)cc6)c6ccc7c(c6)oc6ccccc67)cc5c4c3o2)cc1 ; c1ccc(-c2nc3ccc4ccc5ccc(N(c6cccc([Si](c7ccccc7)(c7ccccc7)c7ccccc7)c6)c6ccc7c(c6)oc6ccccc67)cc5c4c3o2)cc1 ; c1ccc(-c2nc3ccc4ccc5ccc(N(c6ccc([Si](c7ccccc7)(c7ccccc7)c7ccccc7)cc6)c6ccccc6-c6ccccc6)cc5c4c3o2)cc1] 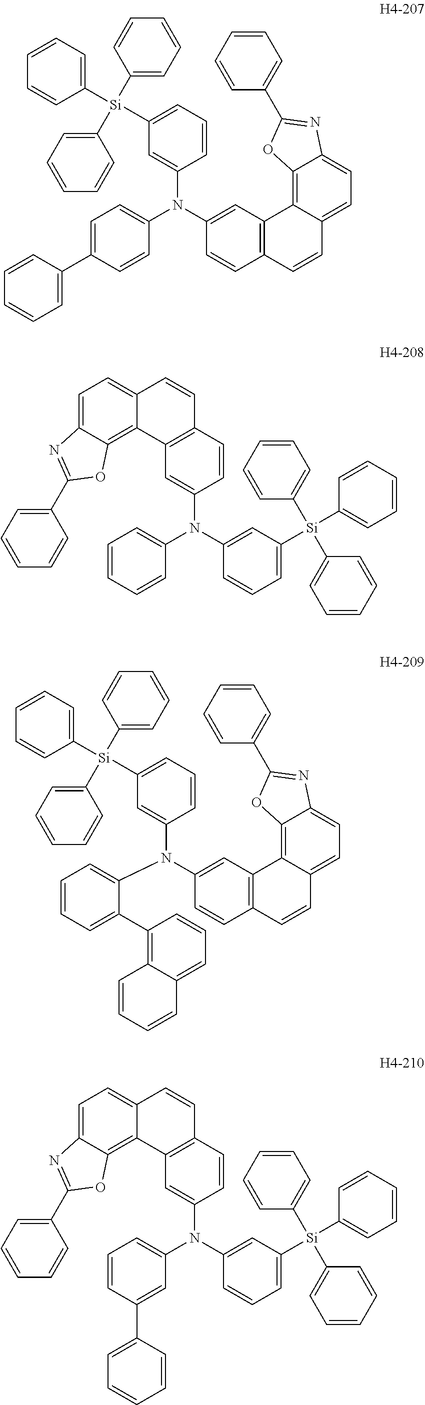 [SMILES: c1ccc(-c2ccc(N(c3cccc([Si](c4ccccc4)(c4ccccc4)c4ccccc4)c3)c3ccc4ccc5ccc6nc(-c7ccccc7)oc6c5c4c3)cc2)cc1 ; c1ccc(-c2nc3ccc4ccc5ccc(N(c6ccccc6)c6cccc([Si](c7ccccc7)(c7ccccc7)c7ccccc7)c6)cc5c4c3o2)cc1 ; c1ccc(-c2nc3ccc4ccc5ccc(N(c6cccc([Si](c7ccccc7)(c7ccccc7)c7ccccc7)c6)c6ccccc6-c6cccc7ccccc67)cc5c4c3o2)cc1 ; c1ccc(-c2cccc(N(c3cccc([Si](c4ccccc4)(c4ccccc4)c4ccccc4)c3)c3ccc4ccc5ccc6nc(-c7ccccc7)oc6c5c4c3)c2)cc1] 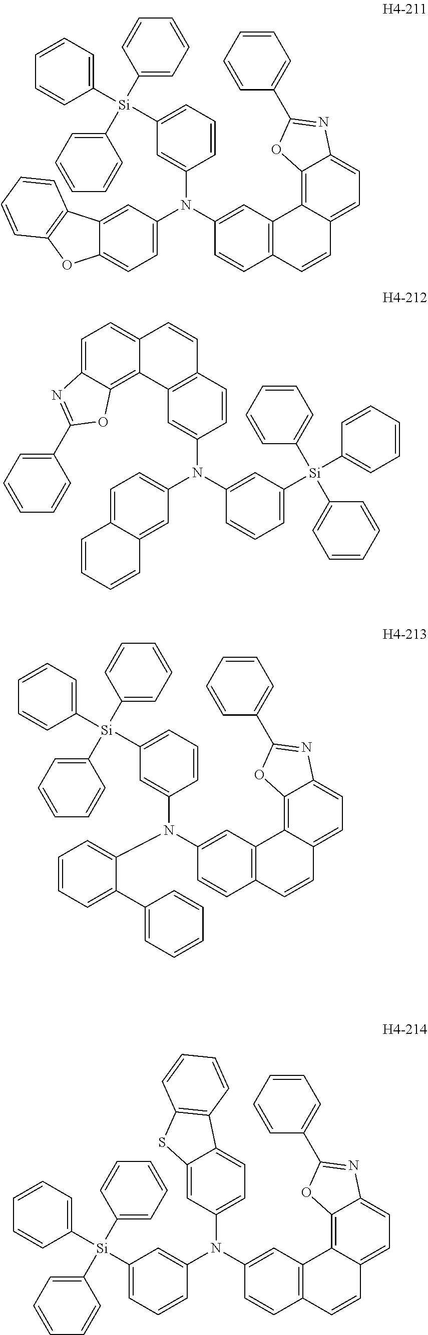 [SMILES: c1ccc(-c2nc3ccc4ccc5ccc(N(c6cccc([Si](c7ccccc7)(c7ccccc7)c7ccccc7)c6)c6ccc7oc8ccccc8c7c6)cc5c4c3o2)cc1 ; c1ccc(-c2nc3ccc4ccc5ccc(N(c6cccc([Si](c7ccccc7)(c7ccccc7)c7ccccc7)c6)c6ccc7ccccc7c6)cc5c4c3o2)cc1 ; c1ccc(-c2nc3ccc4ccc5ccc(N(c6cccc([Si](c7ccccc7)(c7ccccc7)c7ccccc7)c6)c6ccccc6-c6ccccc6)cc5c4c3o2)cc1 ; c1ccc(-c2nc3ccc4ccc5ccc(N(c6cccc([Si](c7ccccc7)(c7ccccc7)c7ccccc7)c6)c6ccc7c(c6)sc6ccccc67)cc5c4c3o2)cc1] 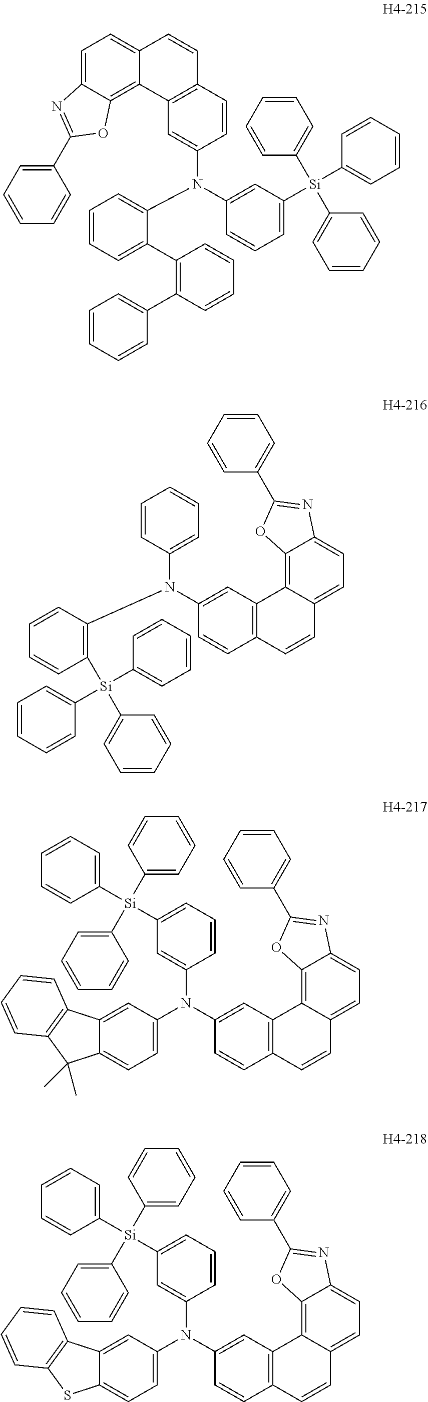 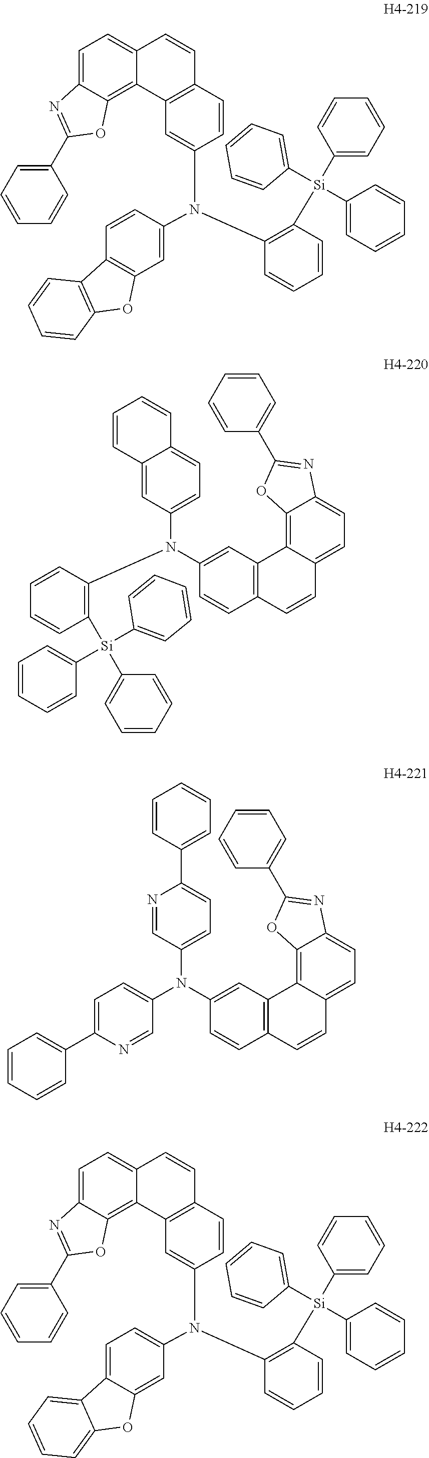 [SMILES: c1ccc(-c2nc3ccc4ccc5ccc(N(c6ccc7c(c6)oc6ccccc67)c6ccccc6[Si](c6ccccc6)(c6ccccc6)c6ccccc6)cc5c4c3o2)cc1 ; c1ccc(-c2nc3ccc4ccc5ccc(N(c6ccc7ccccc7c6)c6ccccc6[Si](c6ccccc6)(c6ccccc6)c6ccccc6)cc5c4c3o2)cc1 ; c1ccc(-c2ccc(N(c3ccc(-c4ccccc4)nc3)c3ccc4ccc5ccc6nc(-c7ccccc7)oc6c5c4c3)cn2)cc1 ; c1ccc(-c2nc3ccc4ccc5ccc(N(c6ccc7c(c6)oc6ccccc67)c6ccccc6[Si](c6ccccc6)(c6ccccc6)c6ccccc6)cc5c4c3o2)cc1] 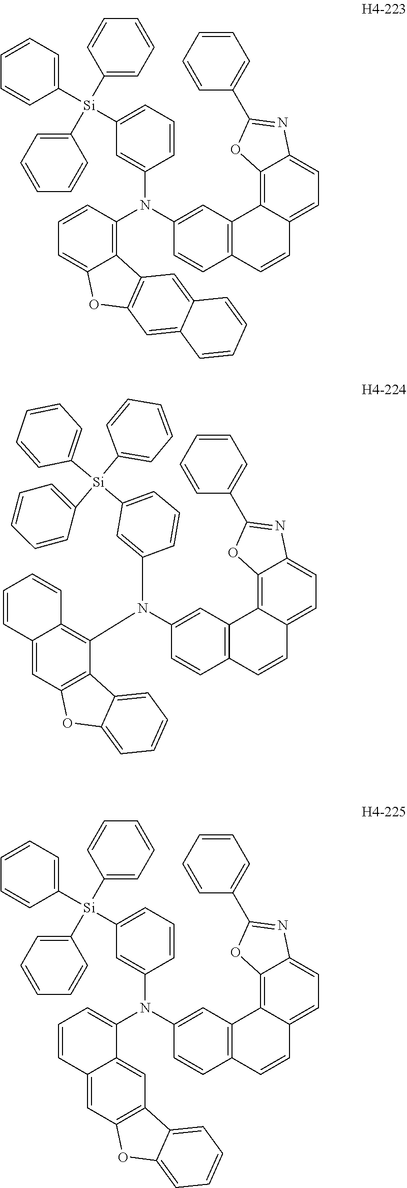 [SMILES: c1ccc(-c2nc3ccc4ccc5ccc(N(c6cccc([Si](c7ccccc7)(c7ccccc7)c7ccccc7)c6)c6cccc7oc8cc9ccccc9cc8c67)cc5c4c3o2)cc1 ; c1ccc(-c2nc3ccc4ccc5ccc(N(c6cccc([Si](c7ccccc7)(c7ccccc7)c7ccccc7)c6)c6c7ccccc7cc7oc8ccccc8c67)cc5c4c3o2)cc1 ; c1ccc(-c2nc3ccc4ccc5ccc(N(c6cccc([Si](c7ccccc7)(c7ccccc7)c7ccccc7)c6)c6cccc7cc8oc9ccccc9c8cc67)cc5c4c3o2)cc1] 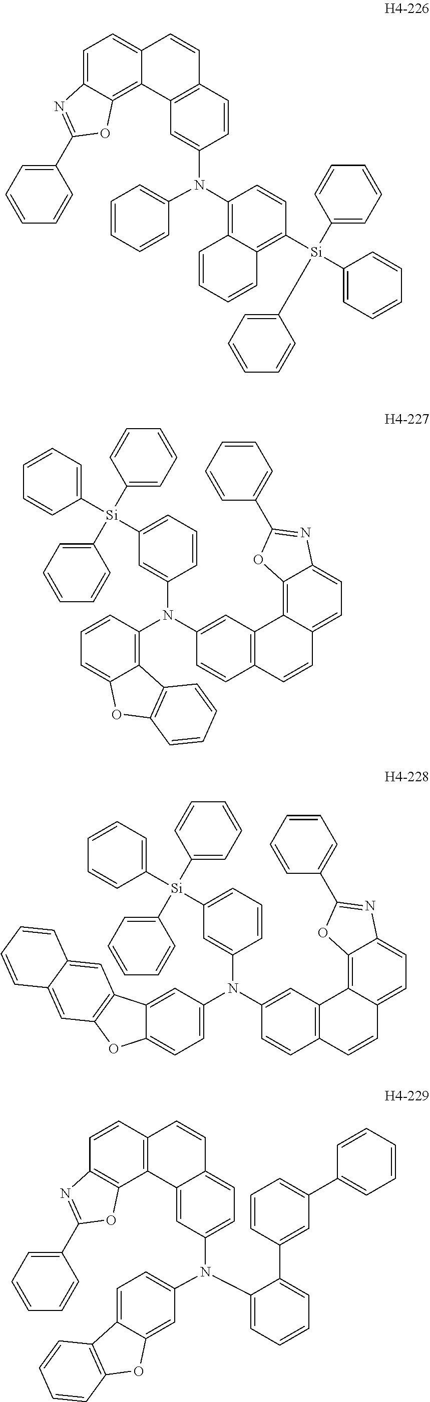 [SMILES: c1ccc(-c2nc3ccc4ccc5ccc(N(c6ccccc6)c6ccc([Si](c7ccccc7)(c7ccccc7)c7ccccc7)c7ccccc67)cc5c4c3o2)cc1 ; c1ccc(-c2nc3ccc4ccc5ccc(N(c6cccc([Si](c7ccccc7)(c7ccccc7)c7ccccc7)c6)c6cccc7oc8ccccc8c67)cc5c4c3o2)cc1 ; c1ccc(-c2nc3ccc4ccc5ccc(N(c6cccc([Si](c7ccccc7)(c7ccccc7)c7ccccc7)c6)c6ccc7oc8cc9ccccc9cc8c7c6)cc5c4c3o2)cc1 ; c1ccc(-c2cccc(-c3ccccc3N(c3ccc4c(c3)oc3ccccc34)c3ccc4ccc5ccc6nc(-c7ccccc7)oc6c5c4c3)c2)cc1] 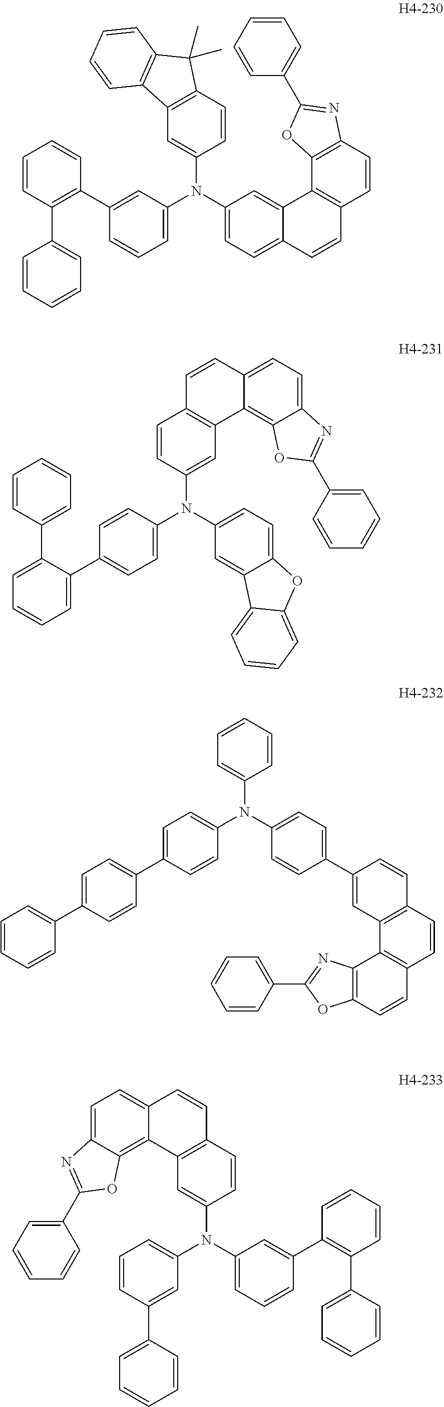 [SMILES: CC1(C)c2ccccc2-c2cc(N(c3cccc(-c4ccccc4-c4ccccc4)c3)c3ccc4ccc5ccc6nc(-c7ccccc7)oc6c5c4c3)ccc21 ; c1ccc(-c2nc3ccc4ccc5ccc(N(c6ccc(-c7ccccc7-c7ccccc7)cc6)c6ccc7oc8ccccc8c7c6)cc5c4c3o2)cc1 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1 ; c1ccc(-c2cccc(N(c3cccc(-c4ccccc4-c4ccccc4)c3)c3ccc4ccc5ccc6nc(-c7ccccc7)oc6c5c4c3)c2)cc1] 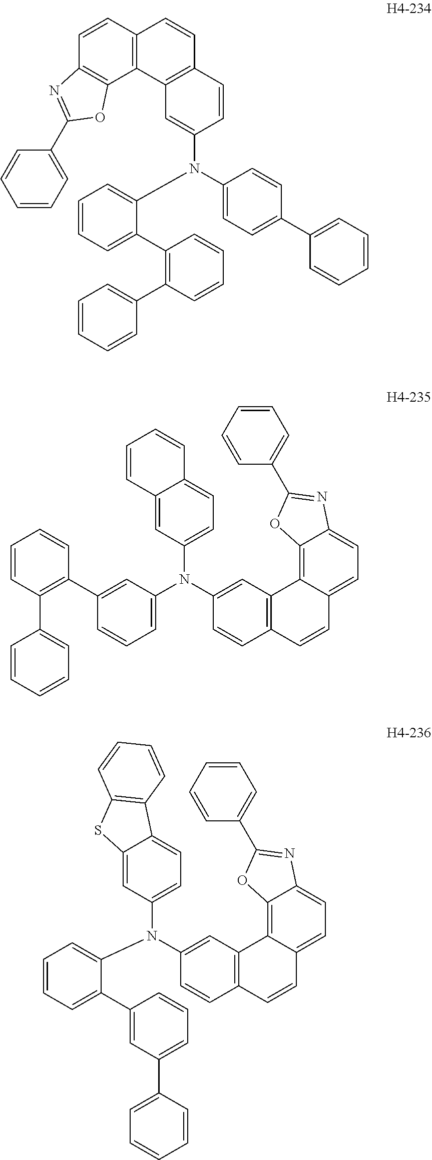 [SMILES: c1ccc(-c2ccc(N(c3ccc4ccc5ccc6nc(-c7ccccc7)oc6c5c4c3)c3ccccc3-c3ccccc3-c3ccccc3)cc2)cc1 ; c1ccc(-c2nc3ccc4ccc5ccc(N(c6cccc(-c7ccccc7-c7ccccc7)c6)c6ccc7ccccc7c6)cc5c4c3o2)cc1 ; c1ccc(-c2cccc(-c3ccccc3N(c3ccc4c(c3)sc3ccccc34)c3ccc4ccc5ccc6nc(-c7ccccc7)oc6c5c4c3)c2)cc1] 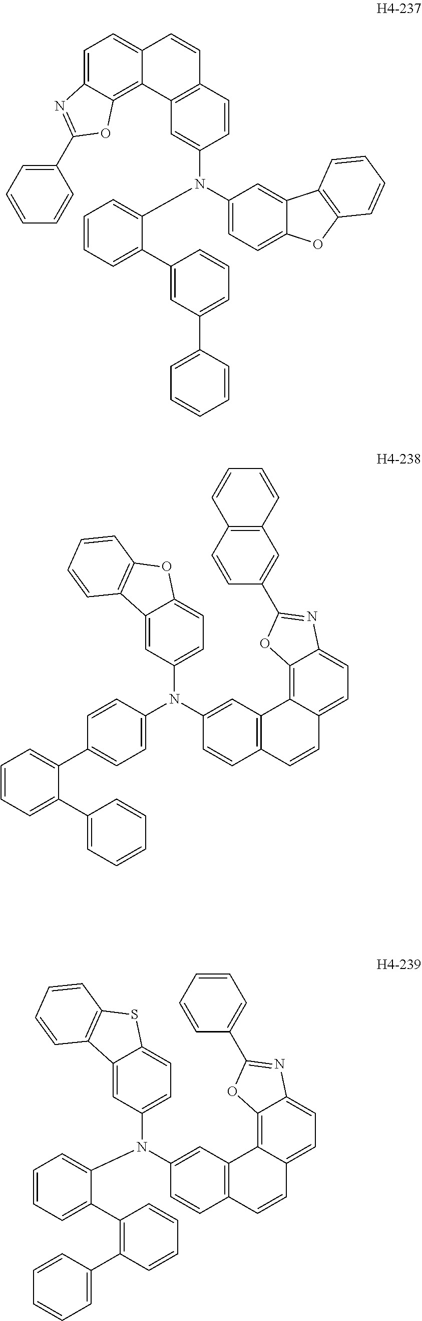 [SMILES: c1ccc(-c2cccc(-c3ccccc3N(c3ccc4oc5ccccc5c4c3)c3ccc4ccc5ccc6nc(-c7ccccc7)oc6c5c4c3)c2)cc1 ; c1ccc(-c2ccccc2-c2ccc(N(c3ccc4oc5ccccc5c4c3)c3ccc4ccc5ccc6nc(-c7ccc8ccccc8c7)oc6c5c4c3)cc2)cc1 ; c1ccc(-c2nc3ccc4ccc5ccc(N(c6ccc7sc8ccccc8c7c6)c6ccccc6-c6ccccc6-c6ccccc6)cc5c4c3o2)cc1] 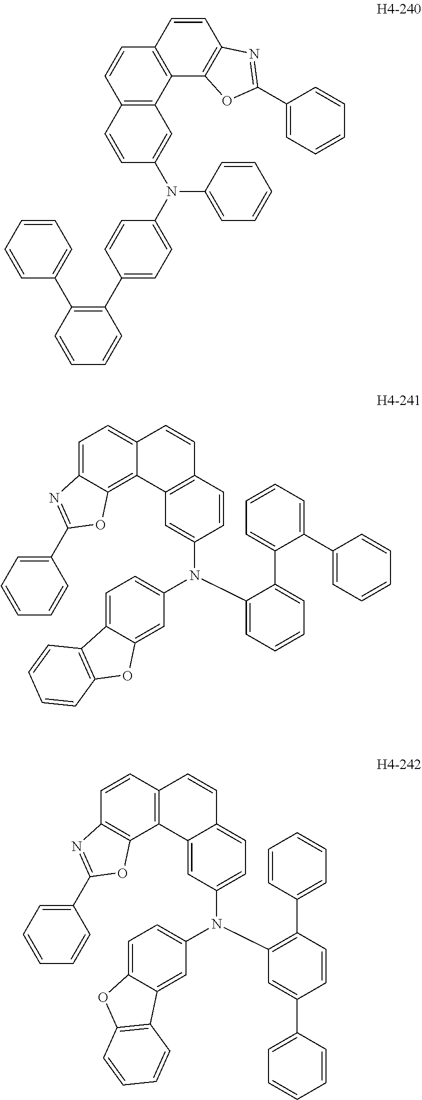 [SMILES: c1ccc(-c2nc3ccc4ccc5ccc(N(c6ccccc6)c6ccc(-c7ccccc7-c7ccccc7)cc6)cc5c4c3o2)cc1 ; c1ccc(-c2nc3ccc4ccc5ccc(N(c6ccc7c(c6)oc6ccccc67)c6ccccc6-c6ccccc6-c6ccccc6)cc5c4c3o2)cc1 ; c1ccc(-c2ccc(-c3ccccc3)c(N(c3ccc4oc5ccccc5c4c3)c3ccc4ccc5ccc6nc(-c7ccccc7)oc6c5c4c3)c2)cc1] 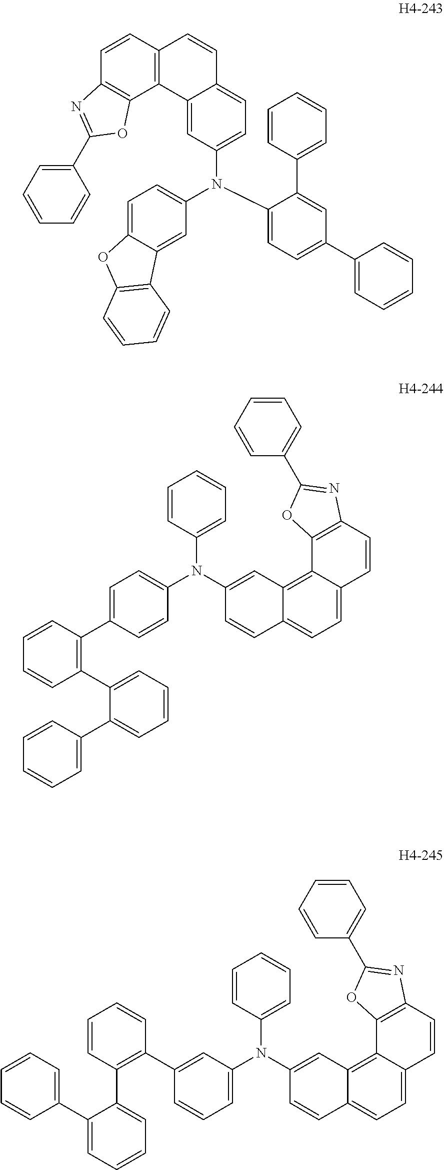 [SMILES: c1ccc(-c2ccc(N(c3ccc4oc5ccccc5c4c3)c3ccc4ccc5ccc6nc(-c7ccccc7)oc6c5c4c3)c(-c3ccccc3)c2)cc1 ; c1ccc(-c2nc3ccc4ccc5ccc(N(c6ccccc6)c6ccc(-c7ccccc7-c7ccccc7-c7ccccc7)cc6)cc5c4c3o2)cc1 ; c1ccc(-c2nc3ccc4ccc5ccc(N(c6ccccc6)c6cccc(-c7ccccc7-c7ccccc7-c7ccccc7)c6)cc5c4c3o2)cc1] 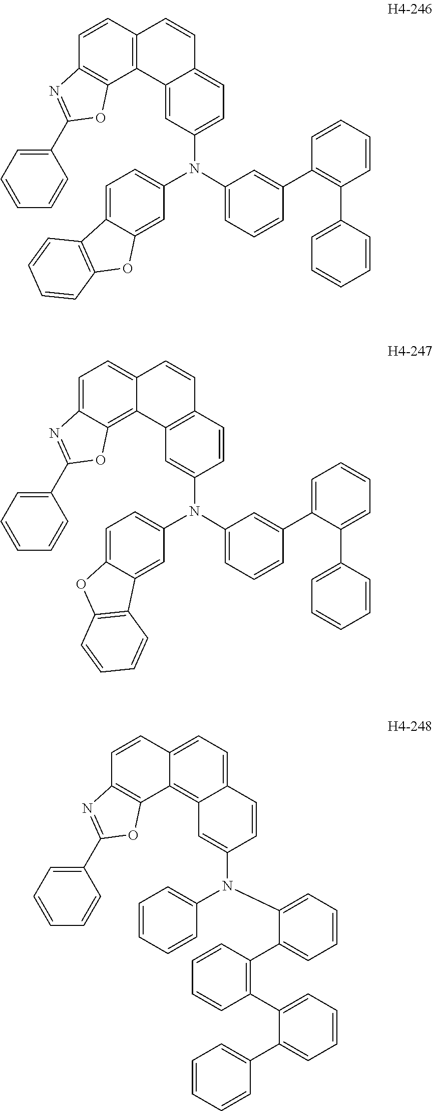 [SMILES: c1ccc(-c2nc3ccc4ccc5ccc(N(c6cccc(-c7ccccc7-c7ccccc7)c6)c6ccc7c(c6)oc6ccccc67)cc5c4c3o2)cc1 ; c1ccc(-c2nc3ccc4ccc5ccc(N(c6cccc(-c7ccccc7-c7ccccc7)c6)c6ccc7oc8ccccc8c7c6)cc5c4c3o2)cc1 ; c1ccc(-c2nc3ccc4ccc5ccc(N(c6ccccc6)c6ccccc6-c6ccccc6-c6ccccc6-c6ccccc6)cc5c4c3o2)cc1] 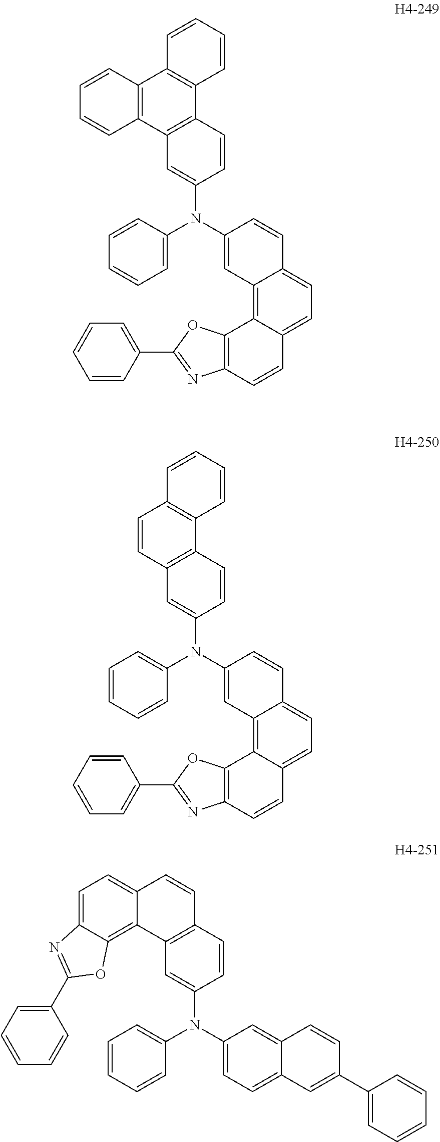 [SMILES: c1ccc(-c2nc3ccc4ccc5ccc(N(c6ccccc6)c6ccc7c8ccccc8c8ccccc8c7c6)cc5c4c3o2)cc1 ; c1ccc(-c2nc3ccc4ccc5ccc(N(c6ccccc6)c6ccc7c(ccc8ccccc87)c6)cc5c4c3o2)cc1 ; c1ccc(-c2ccc3cc(N(c4ccccc4)c4ccc5ccc6ccc7nc(-c8ccccc8)oc7c6c5c4)ccc3c2)cc1] 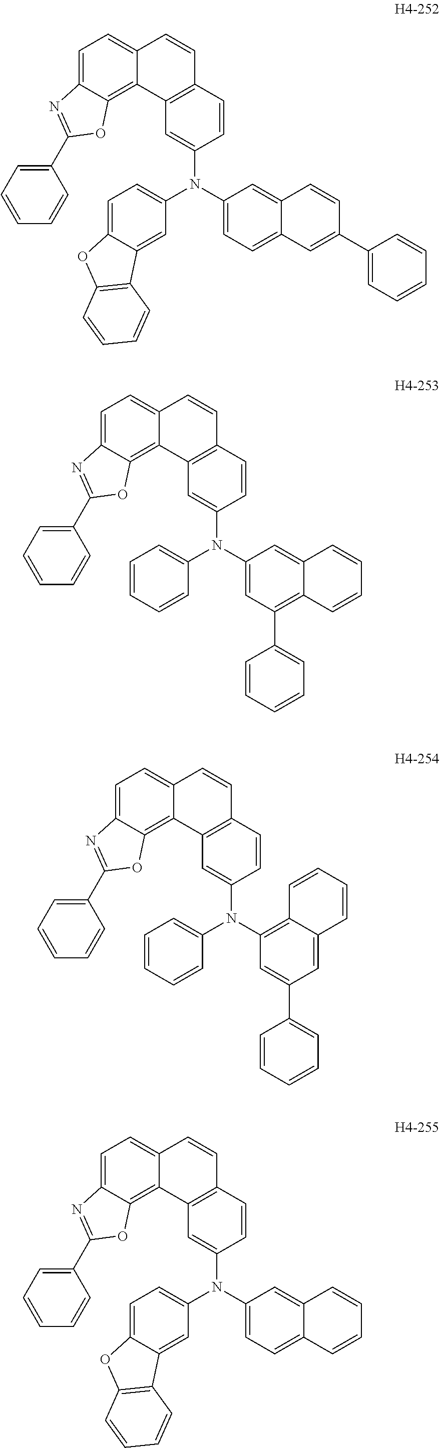 [SMILES: c1ccc(-c2ccc3cc(N(c4ccc5oc6ccccc6c5c4)c4ccc5ccc6ccc7nc(-c8ccccc8)oc7c6c5c4)ccc3c2)cc1 ; c1ccc(-c2nc3ccc4ccc5ccc(N(c6ccccc6)c6cc(-c7ccccc7)c7ccccc7c6)cc5c4c3o2)cc1 ; c1ccc(-c2cc(N(c3ccccc3)c3ccc4ccc5ccc6nc(-c7ccccc7)oc6c5c4c3)c3ccccc3c2)cc1 ; c1ccc(-c2nc3ccc4ccc5ccc(N(c6ccc7ccccc7c6)c6ccc7oc8ccccc8c7c6)cc5c4c3o2)cc1] 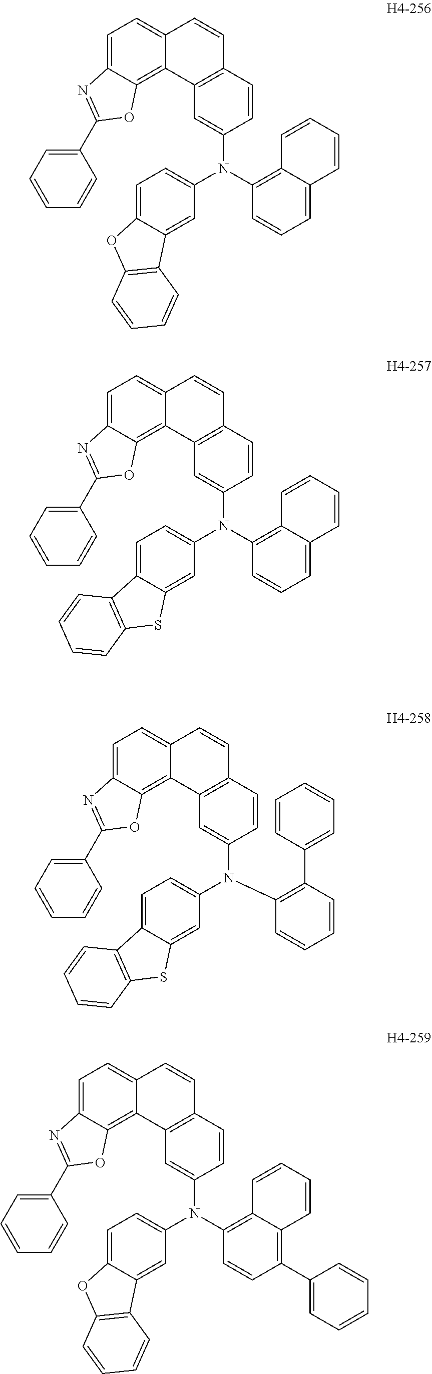 [SMILES: c1ccc(-c2nc3ccc4ccc5ccc(N(c6ccc7oc8ccccc8c7c6)c6cccc7ccccc67)cc5c4c3o2)cc1 ; c1ccc(-c2nc3ccc4ccc5ccc(N(c6ccc7c(c6)sc6ccccc67)c6cccc7ccccc67)cc5c4c3o2)cc1 ; c1ccc(-c2nc3ccc4ccc5ccc(N(c6ccc7c(c6)sc6ccccc67)c6ccccc6-c6ccccc6)cc5c4c3o2)cc1 ; c1ccc(-c2nc3ccc4ccc5ccc(N(c6ccc7oc8ccccc8c7c6)c6ccc(-c7ccccc7)c7ccccc67)cc5c4c3o2)cc1] 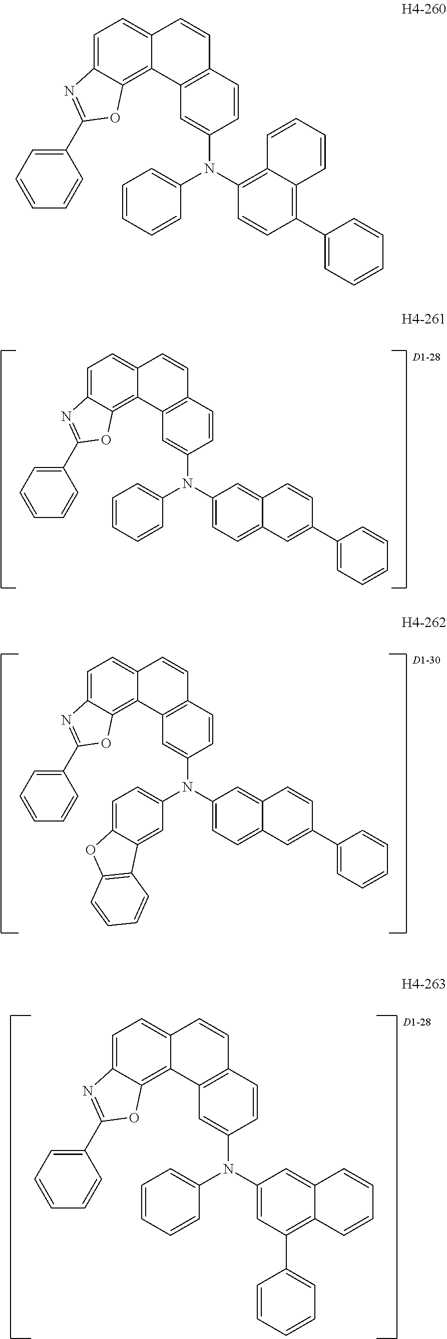 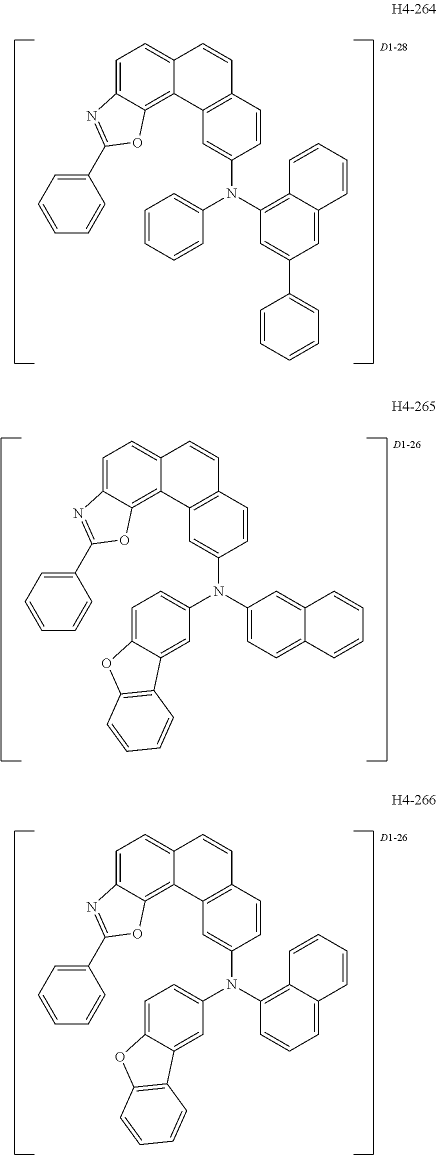 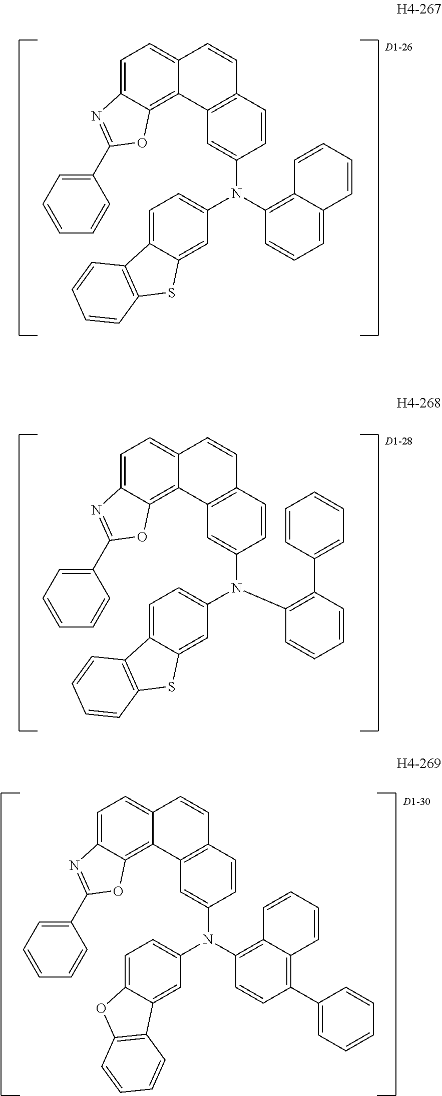 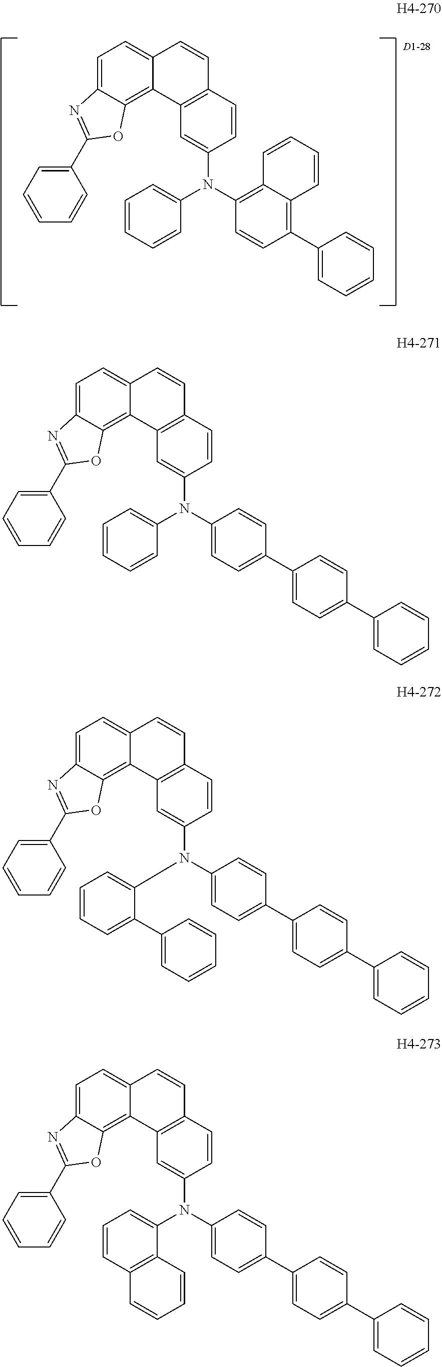 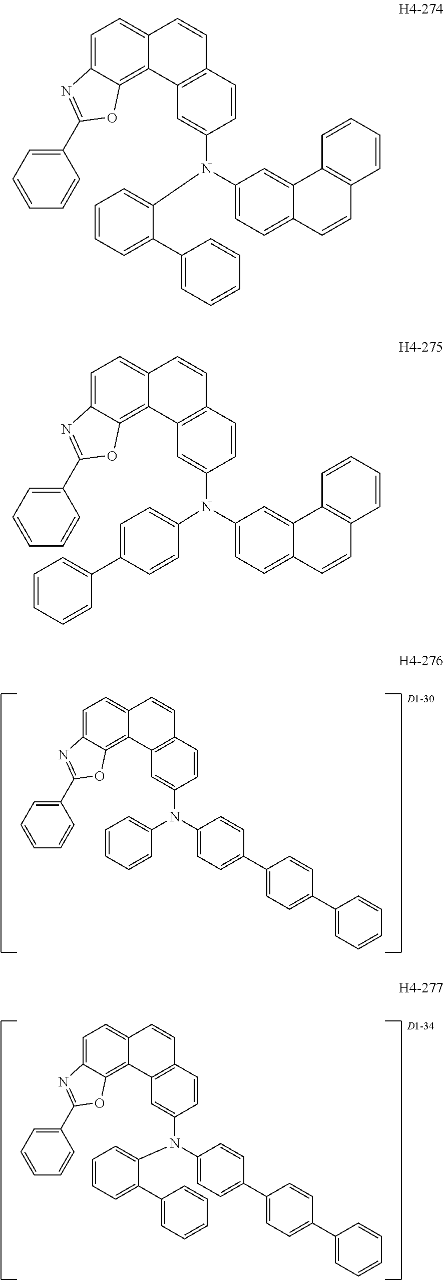 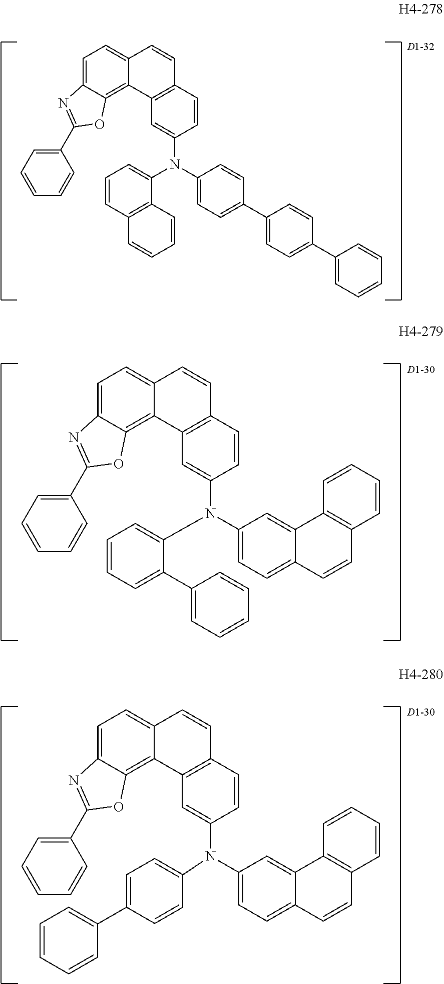 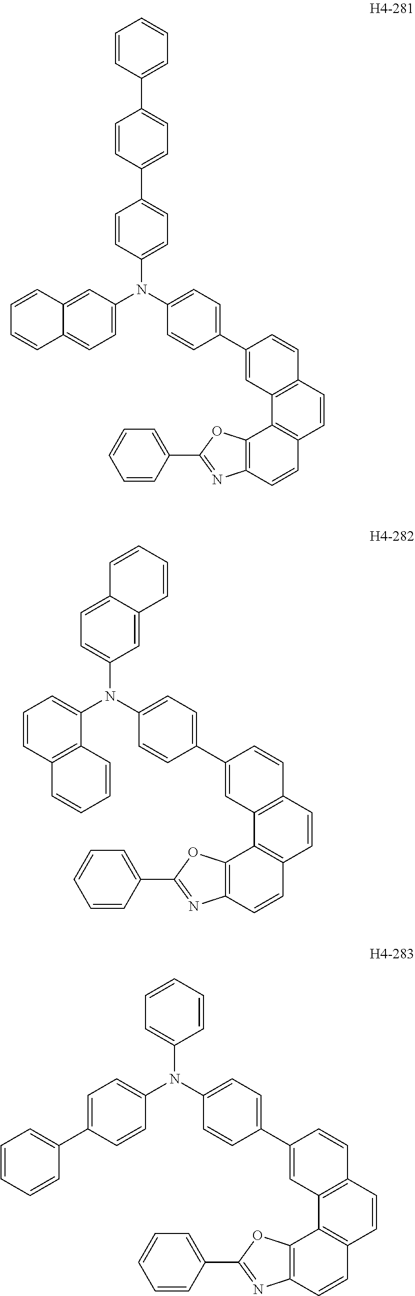 [SMILES: c1ccc(-c2ccc(-c3ccc(N(c4ccc(-c5ccc6ccc7ccc8nc(-c9ccccc9)oc8c7c6c5)cc4)c4ccc5ccccc5c4)cc3)cc2)cc1 ; c1ccc(-c2nc3ccc4ccc5ccc(-c6ccc(N(c7ccc8ccccc8c7)c7cccc8ccccc78)cc6)cc5c4c3o2)cc1 ; c1ccc(-c2ccc(N(c3ccccc3)c3ccc(-c4ccc5ccc6ccc7nc(-c8ccccc8)oc7c6c5c4)cc3)cc2)cc1] 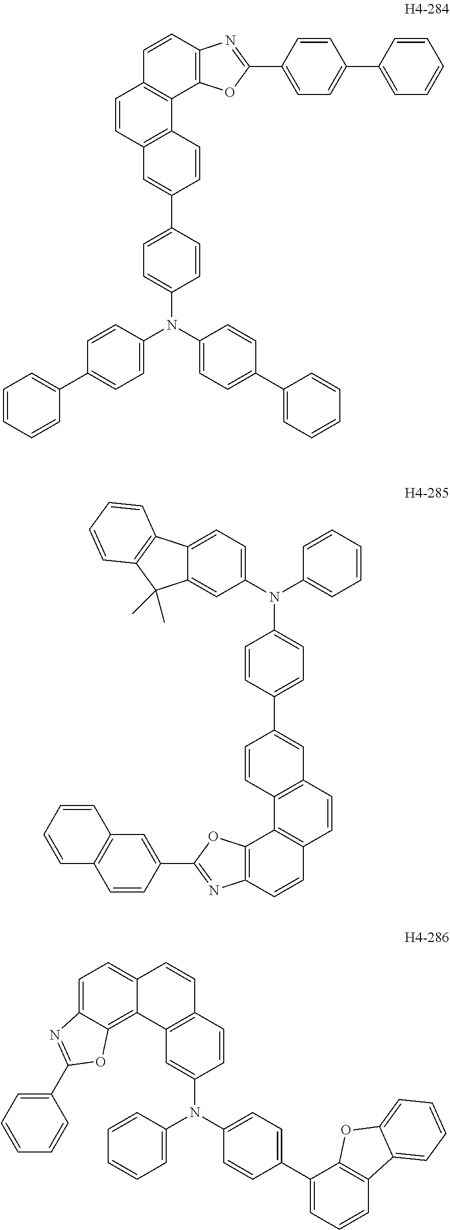 [SMILES: c1ccc(-c2ccc(-c3nc4ccc5ccc6cc(-c7ccc(N(c8ccc(-c9ccccc9)cc8)c8ccc(-c9ccccc9)cc8)cc7)ccc6c5c4o3)cc2)cc1 ; CC1(C)c2ccccc2-c2ccc(N(c3ccccc3)c3ccc(-c4ccc5c(ccc6ccc7nc(-c8ccc9ccccc9c8)oc7c65)c4)cc3)cc21 ; c1ccc(-c2nc3ccc4ccc5ccc(N(c6ccccc6)c6ccc(-c7cccc8c7oc7ccccc78)cc6)cc5c4c3o2)cc1] 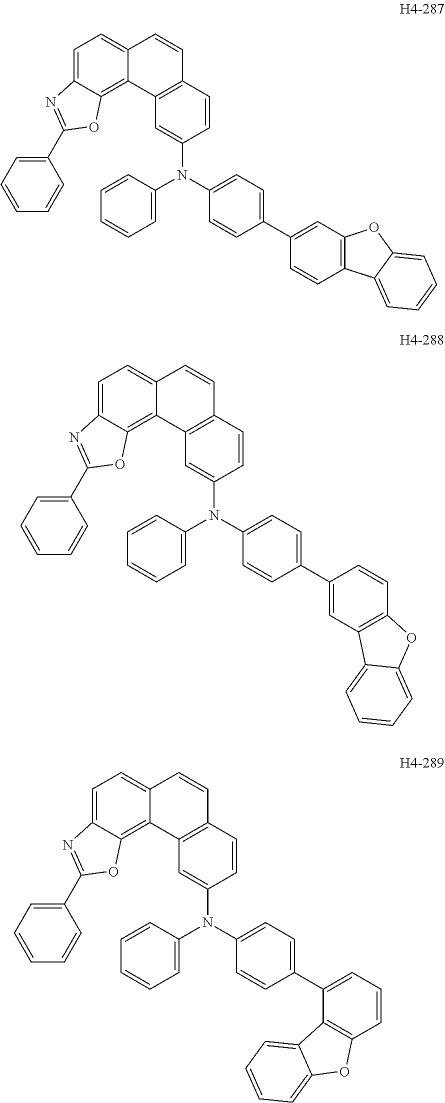 [SMILES: c1ccc(-c2nc3ccc4ccc5ccc(N(c6ccccc6)c6ccc(-c7ccc8c(c7)oc7ccccc78)cc6)cc5c4c3o2)cc1 ; c1ccc(-c2nc3ccc4ccc5ccc(N(c6ccccc6)c6ccc(-c7ccc8oc9ccccc9c8c7)cc6)cc5c4c3o2)cc1 ; c1ccc(-c2nc3ccc4ccc5ccc(N(c6ccccc6)c6ccc(-c7cccc8oc9ccccc9c78)cc6)cc5c4c3o2)cc1] 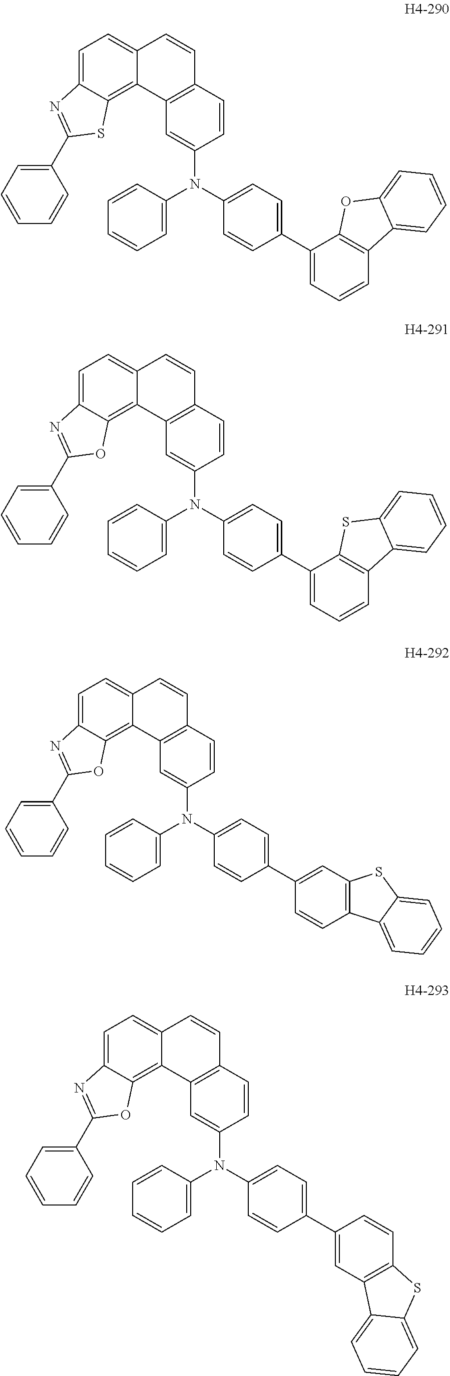 [SMILES: c1ccc(-c2nc3ccc4ccc5ccc(N(c6ccccc6)c6ccc(-c7cccc8c7oc7ccccc78)cc6)cc5c4c3s2)cc1 ; c1ccc(-c2nc3ccc4ccc5ccc(N(c6ccccc6)c6ccc(-c7cccc8c7sc7ccccc78)cc6)cc5c4c3o2)cc1 ; c1ccc(-c2nc3ccc4ccc5ccc(N(c6ccccc6)c6ccc(-c7ccc8c(c7)sc7ccccc78)cc6)cc5c4c3o2)cc1 ; c1ccc(-c2nc3ccc4ccc5ccc(N(c6ccccc6)c6ccc(-c7ccc8sc9ccccc9c8c7)cc6)cc5c4c3o2)cc1] 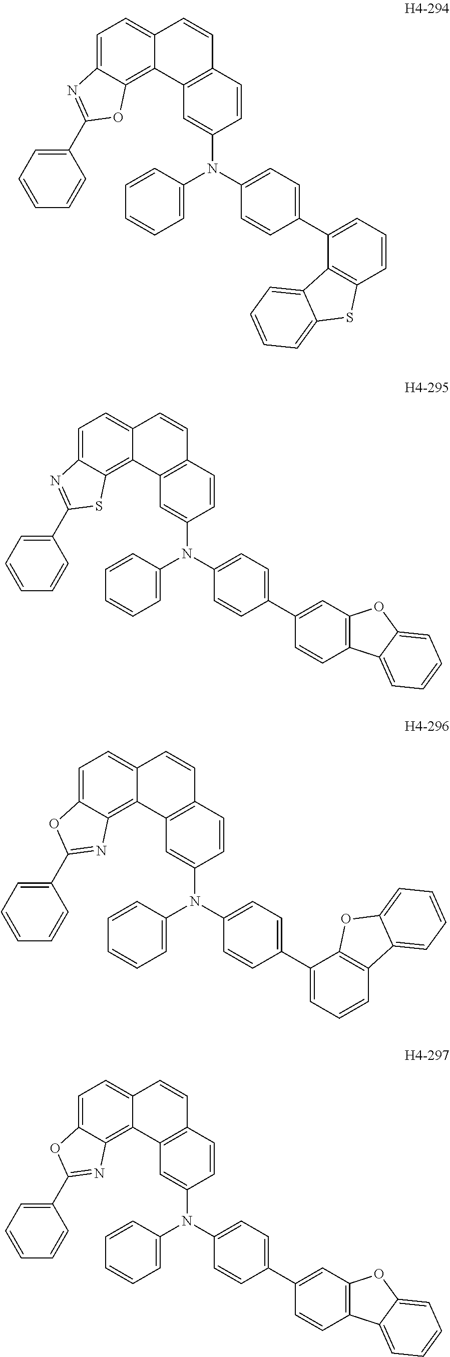 [SMILES: c1ccc(-c2nc3ccc4ccc5ccc(N(c6ccccc6)c6ccc(-c7cccc8sc9ccccc9c78)cc6)cc5c4c3o2)cc1 ; c1ccc(-c2nc3ccc4ccc5ccc(N(c6ccccc6)c6ccc(-c7ccc8c(c7)oc7ccccc78)cc6)cc5c4c3s2)cc1 ; c1ccc(-c2nc3c(ccc4ccc5ccc(N(c6ccccc6)c6ccc(-c7cccc8c7oc7ccccc78)cc6)cc5c43)o2)cc1 ; c1ccc(-c2nc3c(ccc4ccc5ccc(N(c6ccccc6)c6ccc(-c7ccc8c(c7)oc7ccccc78)cc6)cc5c43)o2)cc1] 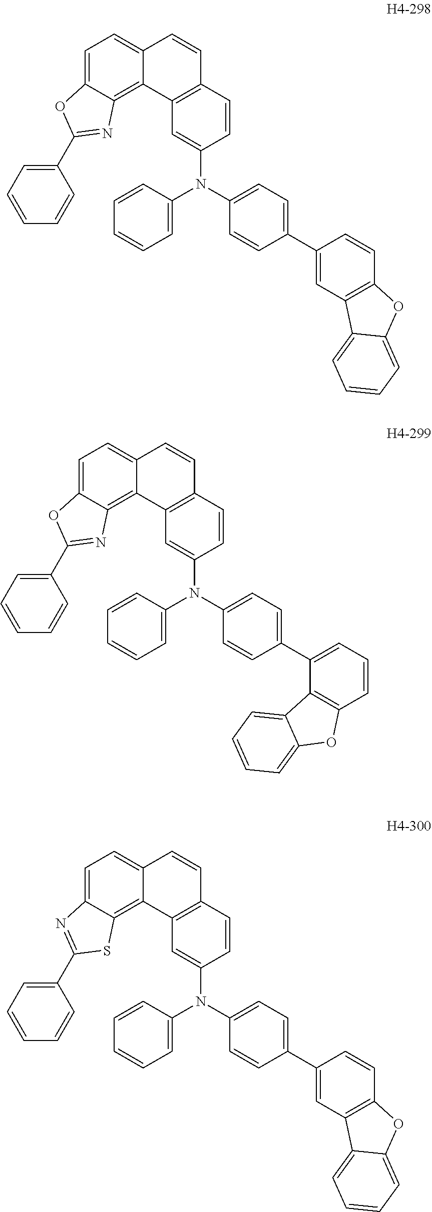 [SMILES: c1ccc(-c2nc3c(ccc4ccc5ccc(N(c6ccccc6)c6ccc(-c7ccc8oc9ccccc9c8c7)cc6)cc5c43)o2)cc1 ; c1ccc(-c2nc3c(ccc4ccc5ccc(N(c6ccccc6)c6ccc(-c7cccc8oc9ccccc9c78)cc6)cc5c43)o2)cc1 ; c1ccc(-c2nc3ccc4ccc5ccc(N(c6ccccc6)c6ccc(-c7ccc8oc9ccccc9c8c7)cc6)cc5c4c3s2)cc1] 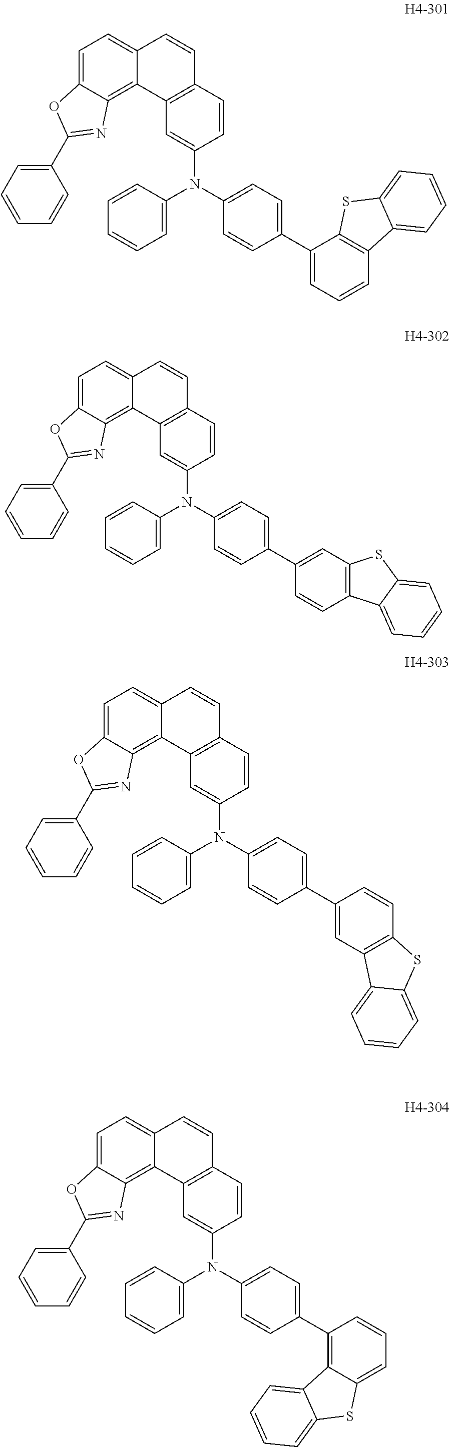 [SMILES: c1ccc(-c2nc3c(ccc4ccc5ccc(N(c6ccccc6)c6ccc(-c7cccc8c7sc7ccccc78)cc6)cc5c43)o2)cc1 ; c1ccc(-c2nc3c(ccc4ccc5ccc(N(c6ccccc6)c6ccc(-c7ccc8c(c7)sc7ccccc78)cc6)cc5c43)o2)cc1 ; c1ccc(-c2nc3c(ccc4ccc5ccc(N(c6ccccc6)c6ccc(-c7ccc8sc9ccccc9c8c7)cc6)cc5c43)o2)cc1 ; c1ccc(-c2nc3c(ccc4ccc5ccc(N(c6ccccc6)c6ccc(-c7cccc8sc9ccccc9c78)cc6)cc5c43)o2)cc1] 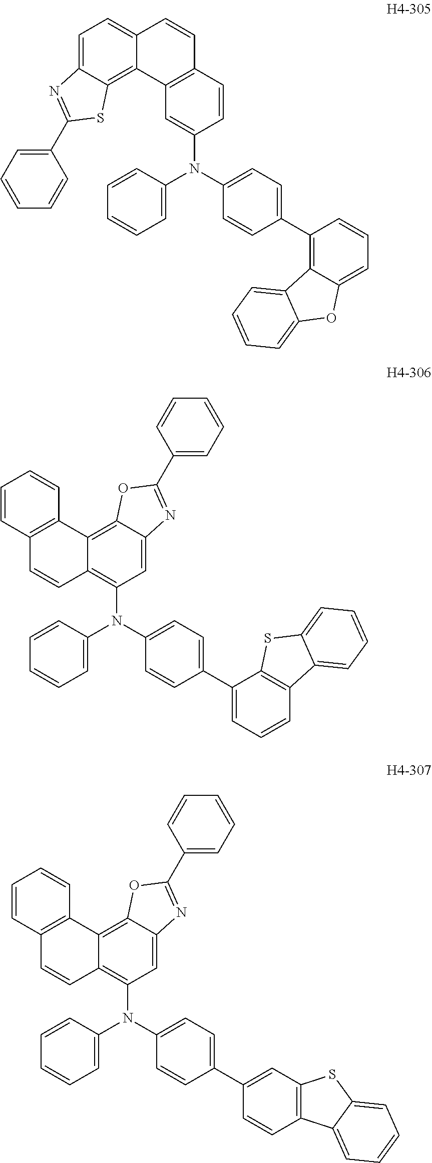 [SMILES: c1ccc(-c2nc3ccc4ccc5ccc(N(c6ccccc6)c6ccc(-c7cccc8oc9ccccc9c78)cc6)cc5c4c3s2)cc1 ; c1ccc(-c2nc3cc(N(c4ccccc4)c4ccc(-c5cccc6c5sc5ccccc56)cc4)c4ccc5ccccc5c4c3o2)cc1 ; c1ccc(-c2nc3cc(N(c4ccccc4)c4ccc(-c5ccc6c(c5)sc5ccccc56)cc4)c4ccc5ccccc5c4c3o2)cc1] 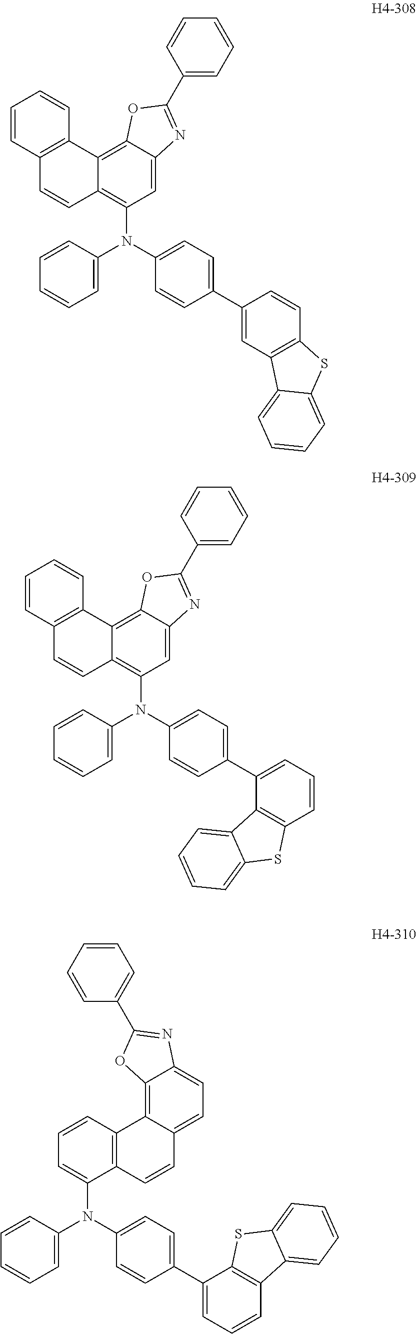 [SMILES: c1ccc(-c2nc3cc(N(c4ccccc4)c4ccc(-c5ccc6sc7ccccc7c6c5)cc4)c4ccc5ccccc5c4c3o2)cc1 ; c1ccc(-c2nc3cc(N(c4ccccc4)c4ccc(-c5cccc6sc7ccccc7c56)cc4)c4ccc5ccccc5c4c3o2)cc1 ; c1ccc(-c2nc3ccc4ccc5c(N(c6ccccc6)c6ccc(-c7cccc8c7sc7ccccc78)cc6)cccc5c4c3o2)cc1] 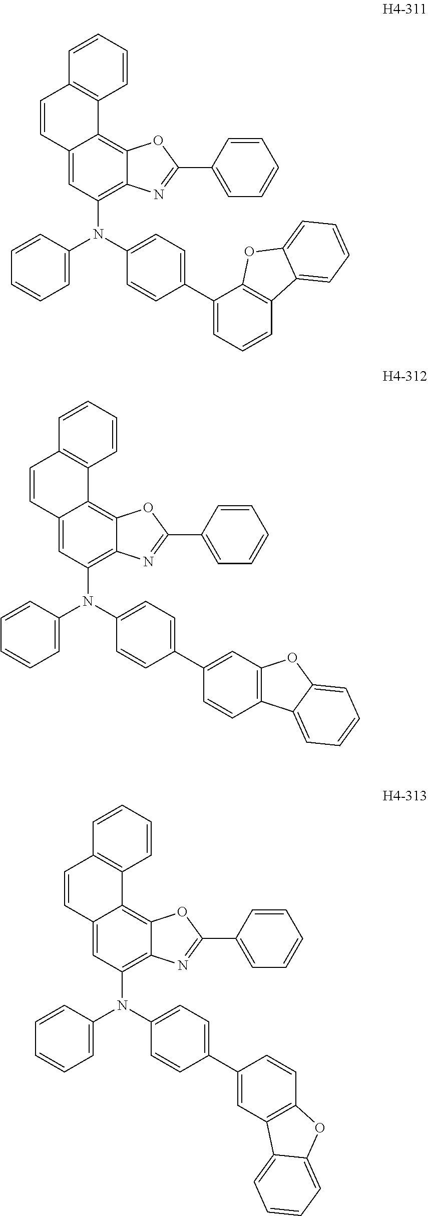 [SMILES: c1ccc(-c2nc3c(N(c4ccccc4)c4ccc(-c5cccc6c5oc5ccccc56)cc4)cc4ccc5ccccc5c4c3o2)cc1 ; c1ccc(-c2nc3c(N(c4ccccc4)c4ccc(-c5ccc6c(c5)oc5ccccc56)cc4)cc4ccc5ccccc5c4c3o2)cc1 ; c1ccc(-c2nc3c(N(c4ccccc4)c4ccc(-c5ccc6oc7ccccc7c6c5)cc4)cc4ccc5ccccc5c4c3o2)cc1] 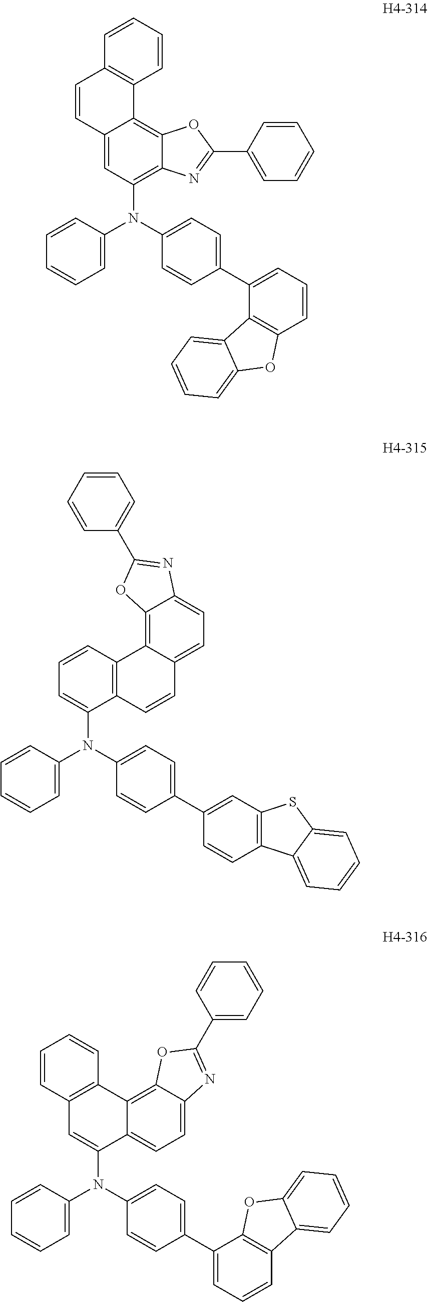 [SMILES: c1ccc(-c2nc3c(N(c4ccccc4)c4ccc(-c5cccc6oc7ccccc7c56)cc4)cc4ccc5ccccc5c4c3o2)cc1 ; c1ccc(-c2nc3ccc4ccc5c(N(c6ccccc6)c6ccc(-c7ccc8c(c7)sc7ccccc78)cc6)cccc5c4c3o2)cc1 ; c1ccc(-c2nc3ccc4c(N(c5ccccc5)c5ccc(-c6cccc7c6oc6ccccc67)cc5)cc5ccccc5c4c3o2)cc1] 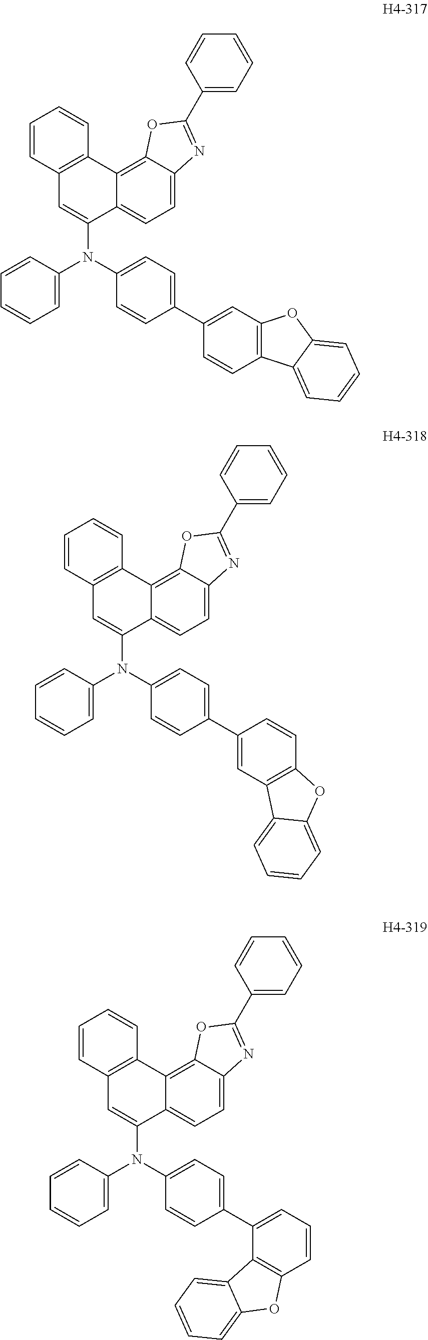 [SMILES: c1ccc(-c2nc3ccc4c(N(c5ccccc5)c5ccc(-c6ccc7c(c6)oc6ccccc67)cc5)cc5ccccc5c4c3o2)cc1 ; c1ccc(-c2nc3ccc4c(N(c5ccccc5)c5ccc(-c6ccc7oc8ccccc8c7c6)cc5)cc5ccccc5c4c3o2)cc1 ; c1ccc(-c2nc3ccc4c(N(c5ccccc5)c5ccc(-c6cccc7oc8ccccc8c67)cc5)cc5ccccc5c4c3o2)cc1] 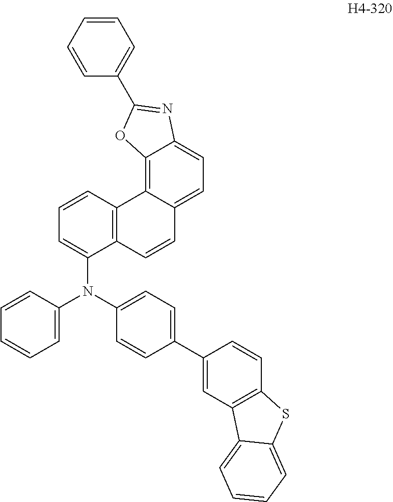 [SMILES: c1ccc(-c2nc3ccc4ccc5c(N(c6ccccc6)c6ccc(-c7ccc8sc9ccccc9c8c7)cc6)cccc5c4c3o2)cc1] 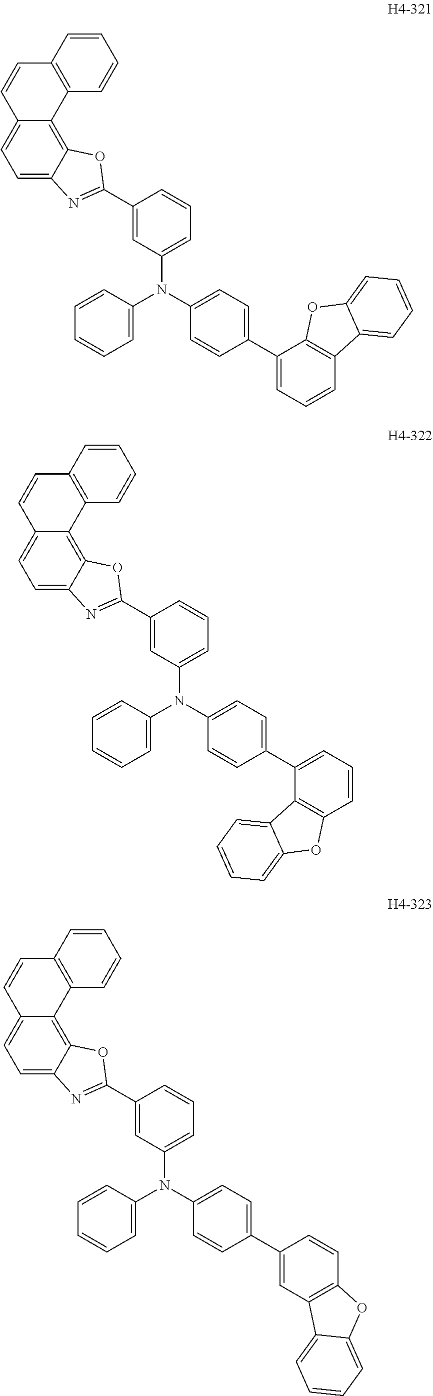 [SMILES: c1ccc(N(c2ccc(-c3cccc4c3oc3ccccc34)cc2)c2cccc(-c3nc4ccc5ccc6ccccc6c5c4o3)c2)cc1 ; c1ccc(N(c2ccc(-c3cccc4oc5ccccc5c34)cc2)c2cccc(-c3nc4ccc5ccc6ccccc6c5c4o3)c2)cc1 ; c1ccc(N(c2ccc(-c3ccc4oc5ccccc5c4c3)cc2)c2cccc(-c3nc4ccc5ccc6ccccc6c5c4o3)c2)cc1] 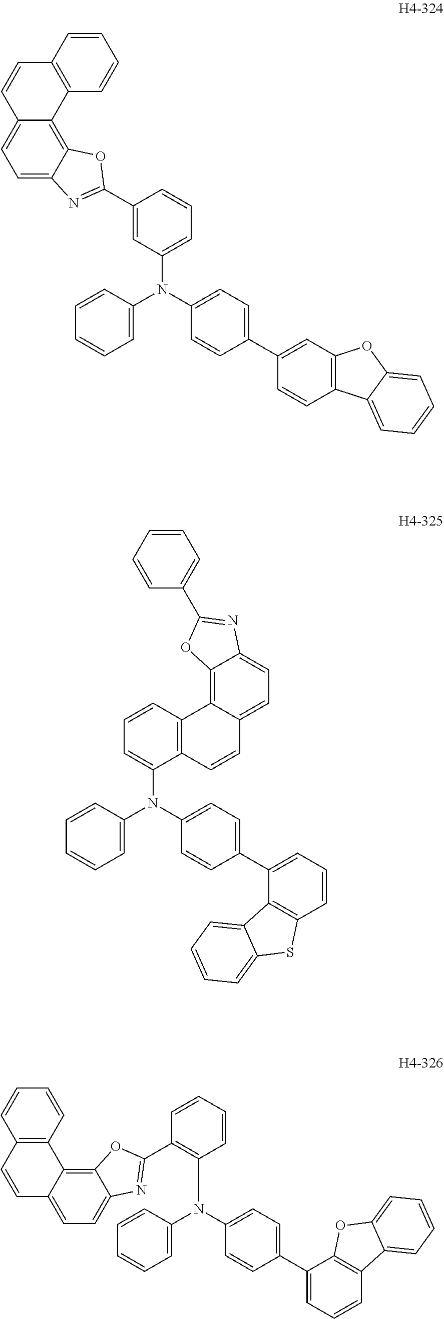 [SMILES: c1ccc(N(c2ccc(-c3ccc4c(c3)oc3ccccc34)cc2)c2cccc(-c3nc4ccc5ccc6ccccc6c5c4o3)c2)cc1 ; c1ccc(-c2nc3ccc4ccc5c(N(c6ccccc6)c6ccc(-c7cccc8sc9ccccc9c78)cc6)cccc5c4c3o2)cc1 ; c1ccc(N(c2ccc(-c3cccc4c3oc3ccccc34)cc2)c2ccccc2-c2nc3ccc4ccc5ccccc5c4c3o2)cc1] 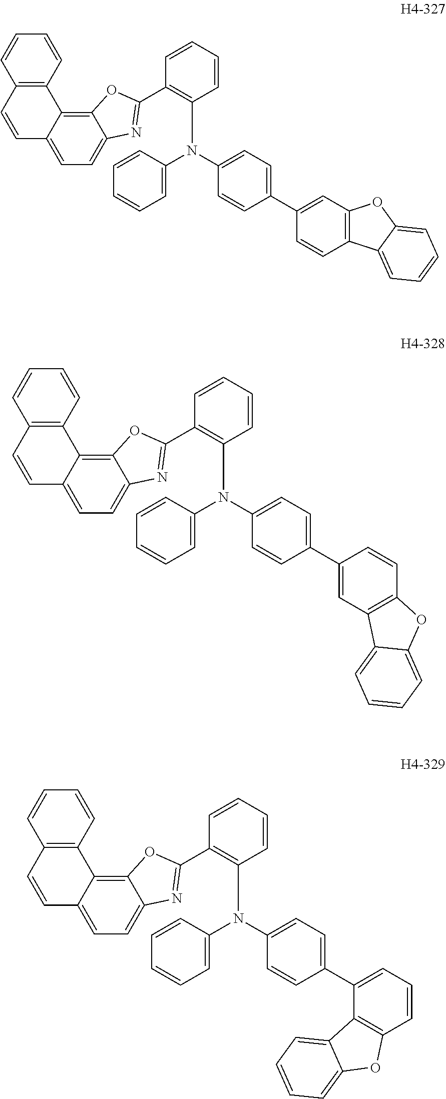 [SMILES: c1ccc(N(c2ccc(-c3ccc4c(c3)oc3ccccc34)cc2)c2ccccc2-c2nc3ccc4ccc5ccccc5c4c3o2)cc1 ; c1ccc(N(c2ccc(-c3ccc4oc5ccccc5c4c3)cc2)c2ccccc2-c2nc3ccc4ccc5ccccc5c4c3o2)cc1 ; c1ccc(N(c2ccc(-c3cccc4oc5ccccc5c34)cc2)c2ccccc2-c2nc3ccc4ccc5ccccc5c4c3o2)cc1] 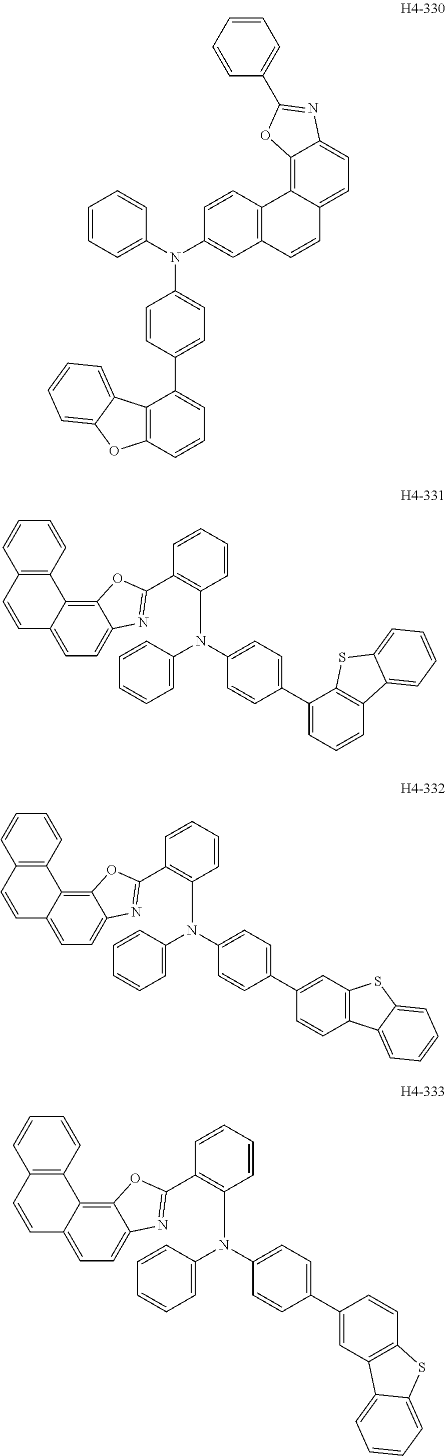 [SMILES: c1ccc(-c2nc3ccc4ccc5cc(N(c6ccccc6)c6ccc(-c7cccc8oc9ccccc9c78)cc6)ccc5c4c3o2)cc1 ; c1ccc(N(c2ccc(-c3cccc4c3sc3ccccc34)cc2)c2ccccc2-c2nc3ccc4ccc5ccccc5c4c3o2)cc1 ; c1ccc(N(c2ccc(-c3ccc4c(c3)sc3ccccc34)cc2)c2ccccc2-c2nc3ccc4ccc5ccccc5c4c3o2)cc1 ; c1ccc(N(c2ccc(-c3ccc4sc5ccccc5c4c3)cc2)c2ccccc2-c2nc3ccc4ccc5ccccc5c4c3o2)cc1] 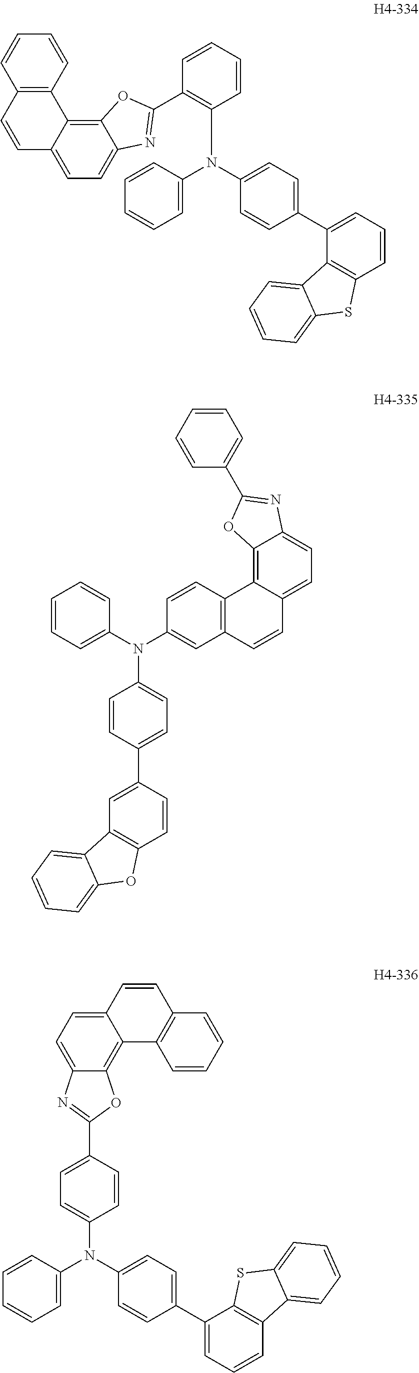 [SMILES: c1ccc(N(c2ccc(-c3cccc4sc5ccccc5c34)cc2)c2ccccc2-c2nc3ccc4ccc5ccccc5c4c3o2)cc1 ; c1ccc(-c2nc3ccc4ccc5cc(N(c6ccccc6)c6ccc(-c7ccc8oc9ccccc9c8c7)cc6)ccc5c4c3o2)cc1 ; c1ccc(N(c2ccc(-c3nc4ccc5ccc6ccccc6c5c4o3)cc2)c2ccc(-c3cccc4c3sc3ccccc34)cc2)cc1] 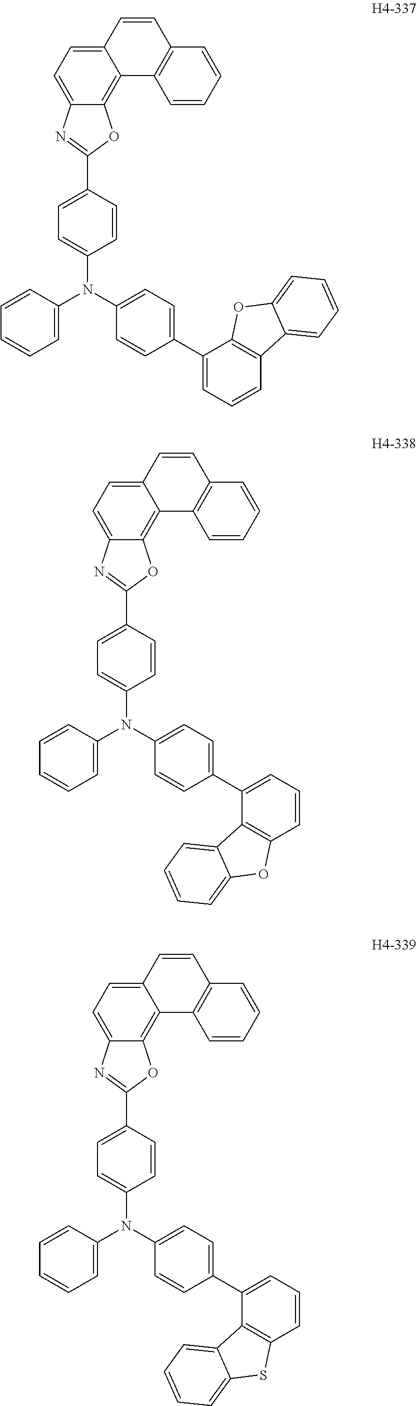 [SMILES: c1ccc(N(c2ccc(-c3nc4ccc5ccc6ccccc6c5c4o3)cc2)c2ccc(-c3cccc4c3oc3ccccc34)cc2)cc1 ; c1ccc(N(c2ccc(-c3nc4ccc5ccc6ccccc6c5c4o3)cc2)c2ccc(-c3cccc4oc5ccccc5c34)cc2)cc1 ; c1ccc(N(c2ccc(-c3nc4ccc5ccc6ccccc6c5c4o3)cc2)c2ccc(-c3cccc4sc5ccccc5c34)cc2)cc1] 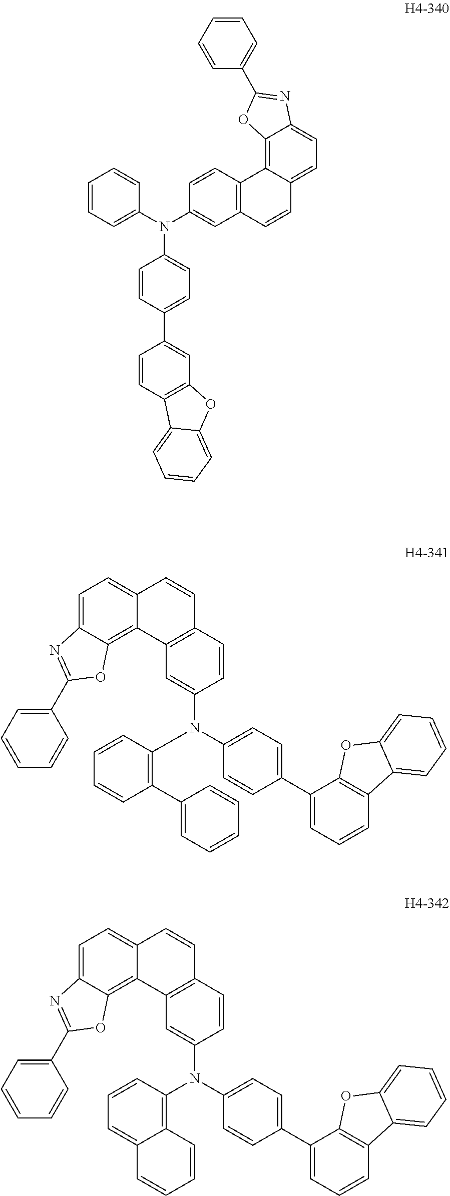 [SMILES: c1ccc(-c2nc3ccc4ccc5cc(N(c6ccccc6)c6ccc(-c7ccc8c(c7)oc7ccccc78)cc6)ccc5c4c3o2)cc1 ; c1ccc(-c2nc3ccc4ccc5ccc(N(c6ccc(-c7cccc8c7oc7ccccc78)cc6)c6ccccc6-c6ccccc6)cc5c4c3o2)cc1 ; c1ccc(-c2nc3ccc4ccc5ccc(N(c6ccc(-c7cccc8c7oc7ccccc78)cc6)c6cccc7ccccc67)cc5c4c3o2)cc1] 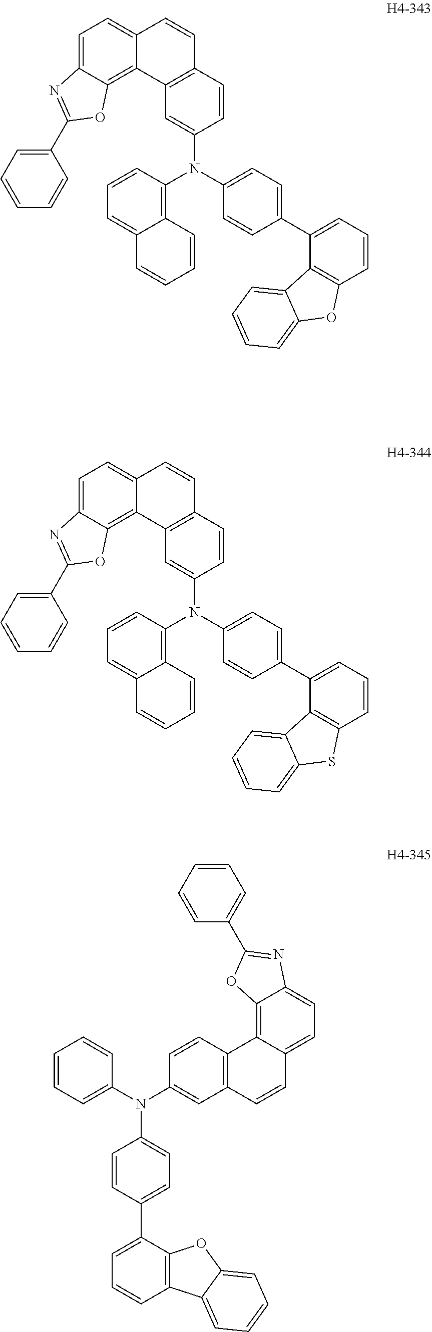 [SMILES: c1ccc(-c2nc3ccc4ccc5ccc(N(c6ccc(-c7cccc8oc9ccccc9c78)cc6)c6cccc7ccccc67)cc5c4c3o2)cc1 ; c1ccc(-c2nc3ccc4ccc5ccc(N(c6ccc(-c7cccc8sc9ccccc9c78)cc6)c6cccc7ccccc67)cc5c4c3o2)cc1 ; c1ccc(-c2nc3ccc4ccc5cc(N(c6ccccc6)c6ccc(-c7cccc8c7oc7ccccc78)cc6)ccc5c4c3o2)cc1] 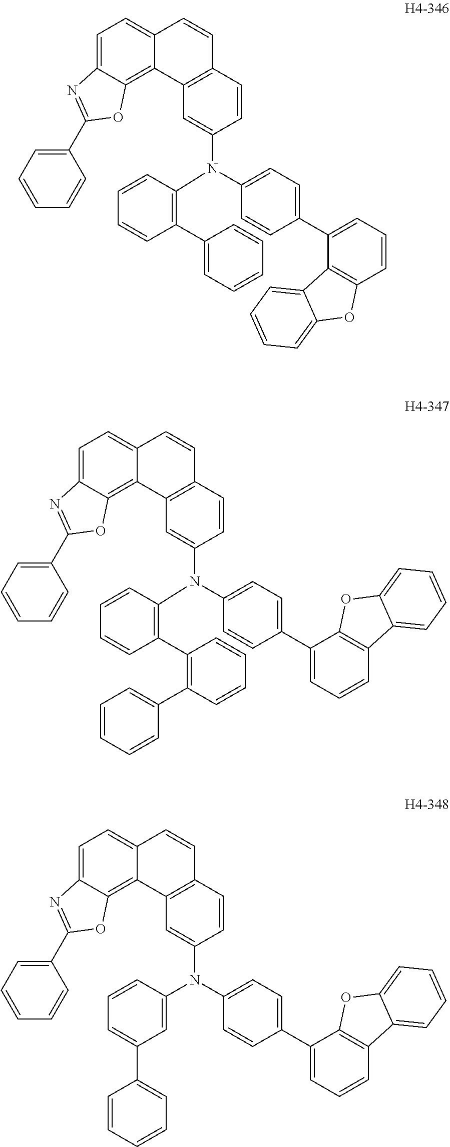 [SMILES: c1ccc(-c2nc3ccc4ccc5ccc(N(c6ccc(-c7cccc8oc9ccccc9c78)cc6)c6ccccc6-c6ccccc6)cc5c4c3o2)cc1 ; c1ccc(-c2nc3ccc4ccc5ccc(N(c6ccc(-c7cccc8c7oc7ccccc78)cc6)c6ccccc6-c6ccccc6-c6ccccc6)cc5c4c3o2)cc1 ; c1ccc(-c2cccc(N(c3ccc(-c4cccc5c4oc4ccccc45)cc3)c3ccc4ccc5ccc6nc(-c7ccccc7)oc6c5c4c3)c2)cc1] 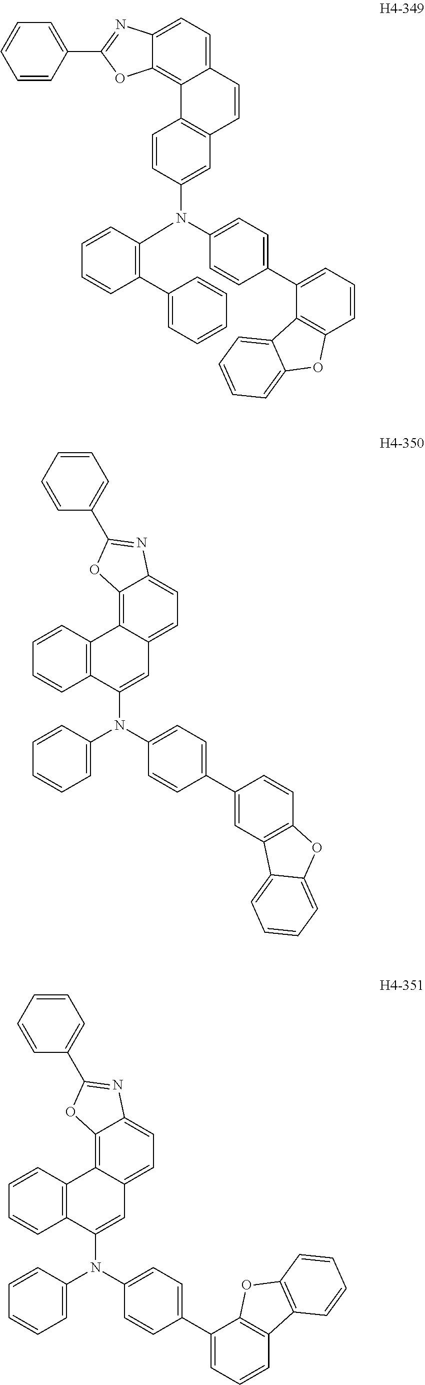 [SMILES: c1ccc(-c2nc3ccc4ccc5cc(N(c6ccc(-c7cccc8oc9ccccc9c78)cc6)c6ccccc6-c6ccccc6)ccc5c4c3o2)cc1 ; c1ccc(-c2nc3ccc4cc(N(c5ccccc5)c5ccc(-c6ccc7oc8ccccc8c7c6)cc5)c5ccccc5c4c3o2)cc1 ; c1ccc(-c2nc3ccc4cc(N(c5ccccc5)c5ccc(-c6cccc7c6oc6ccccc67)cc5)c5ccccc5c4c3o2)cc1] 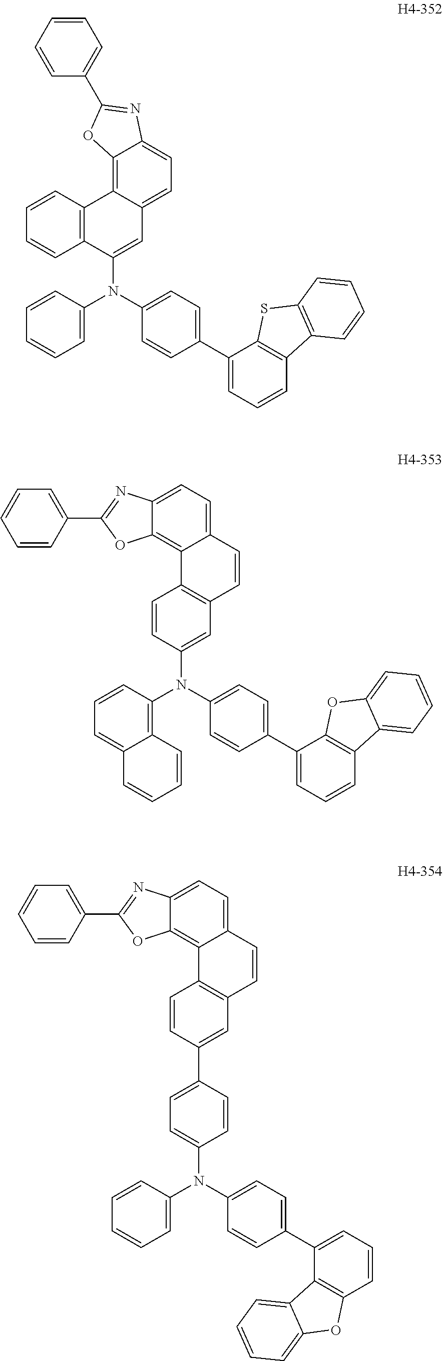 [SMILES: c1ccc(-c2nc3ccc4cc(N(c5ccccc5)c5ccc(-c6cccc7c6sc6ccccc67)cc5)c5ccccc5c4c3o2)cc1 ; c1ccc(-c2nc3ccc4ccc5cc(N(c6ccc(-c7cccc8c7oc7ccccc78)cc6)c6cccc7ccccc67)ccc5c4c3o2)cc1 ; c1ccc(-c2nc3ccc4ccc5cc(-c6ccc(N(c7ccccc7)c7ccc(-c8cccc9oc%10ccccc%10c89)cc7)cc6)ccc5c4c3o2)cc1] 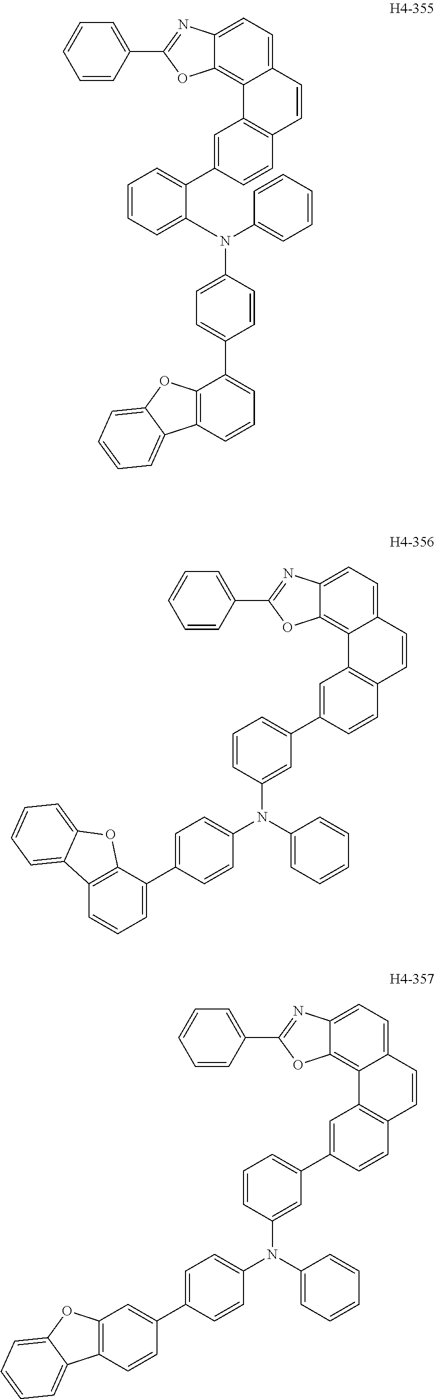 [SMILES: c1ccc(-c2nc3ccc4ccc5ccc(-c6ccccc6N(c6ccccc6)c6ccc(-c7cccc8c7oc7ccccc78)cc6)cc5c4c3o2)cc1 ; c1ccc(-c2nc3ccc4ccc5ccc(-c6cccc(N(c7ccccc7)c7ccc(-c8cccc9c8oc8ccccc89)cc7)c6)cc5c4c3o2)cc1 ; c1ccc(-c2nc3ccc4ccc5ccc(-c6cccc(N(c7ccccc7)c7ccc(-c8ccc9c(c8)oc8ccccc89)cc7)c6)cc5c4c3o2)cc1] 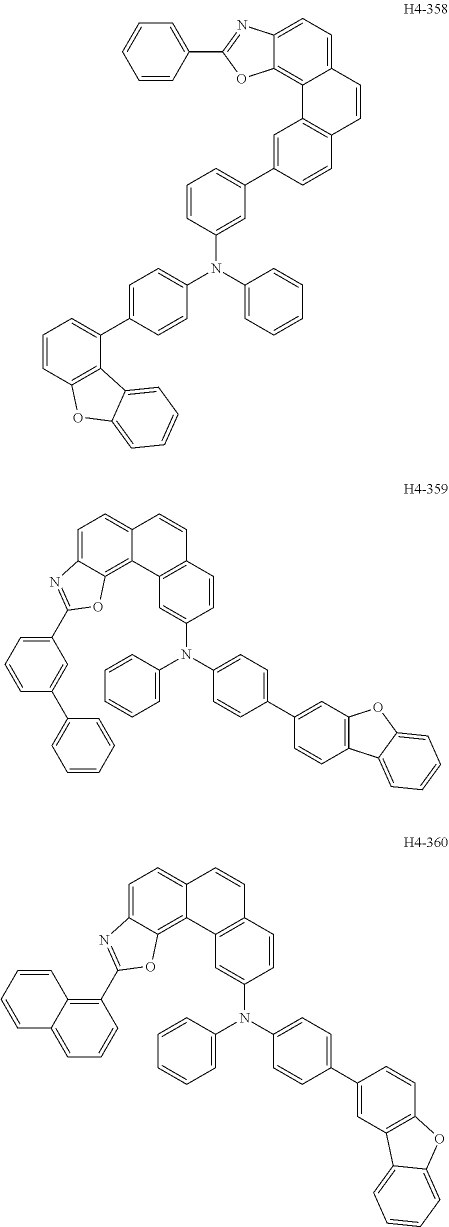 [SMILES: c1ccc(-c2nc3ccc4ccc5ccc(-c6cccc(N(c7ccccc7)c7ccc(-c8cccc9oc%10ccccc%10c89)cc7)c6)cc5c4c3o2)cc1 ; c1ccc(-c2cccc(-c3nc4ccc5ccc6ccc(N(c7ccccc7)c7ccc(-c8ccc9c(c8)oc8ccccc89)cc7)cc6c5c4o3)c2)cc1 ; c1ccc(N(c2ccc(-c3ccc4oc5ccccc5c4c3)cc2)c2ccc3ccc4ccc5nc(-c6cccc7ccccc67)oc5c4c3c2)cc1] 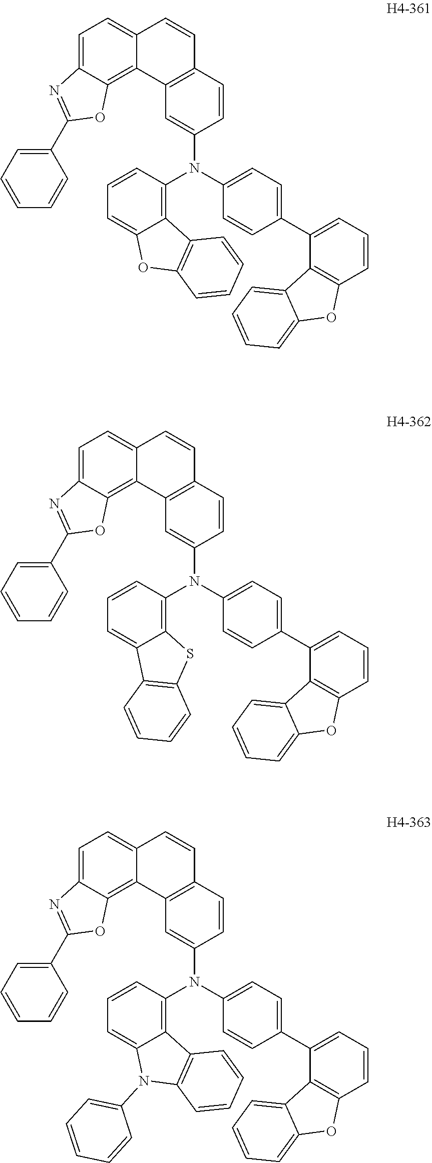 [SMILES: c1ccc(-c2nc3ccc4ccc5ccc(N(c6ccc(-c7cccc8oc9ccccc9c78)cc6)c6cccc7oc8ccccc8c67)cc5c4c3o2)cc1 ; c1ccc(-c2nc3ccc4ccc5ccc(N(c6ccc(-c7cccc8oc9ccccc9c78)cc6)c6cccc7c6sc6ccccc67)cc5c4c3o2)cc1 ; c1ccc(-c2nc3ccc4ccc5ccc(N(c6ccc(-c7cccc8oc9ccccc9c78)cc6)c6cccc7c6c6ccccc6n7-c6ccccc6)cc5c4c3o2)cc1] 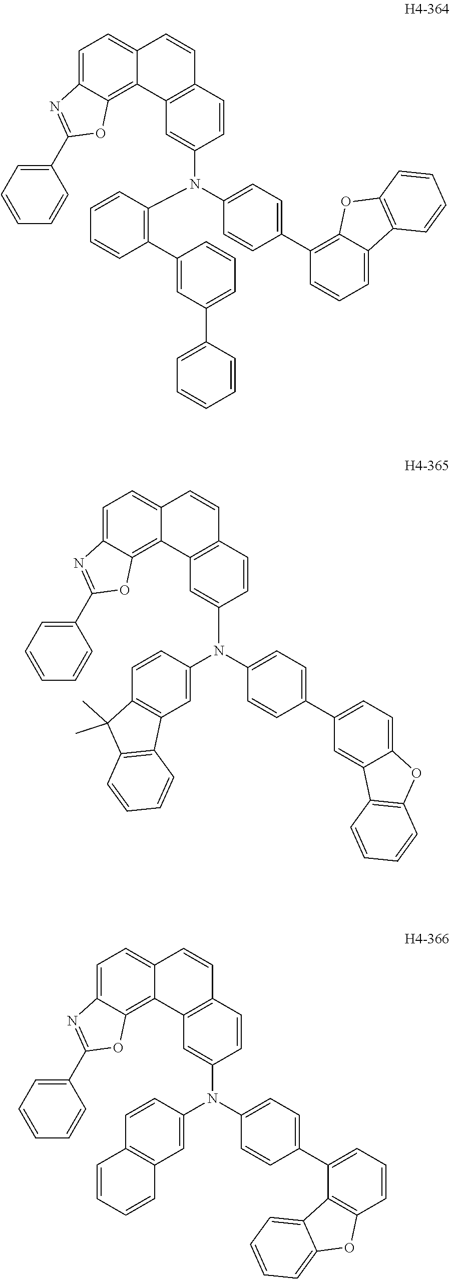 [SMILES: c1ccc(-c2cccc(-c3ccccc3N(c3ccc(-c4cccc5c4oc4ccccc45)cc3)c3ccc4ccc5ccc6nc(-c7ccccc7)oc6c5c4c3)c2)cc1 ; CC1(C)c2ccccc2-c2cc(N(c3ccc(-c4ccc5oc6ccccc6c5c4)cc3)c3ccc4ccc5ccc6nc(-c7ccccc7)oc6c5c4c3)ccc21 ; c1ccc(-c2nc3ccc4ccc5ccc(N(c6ccc(-c7cccc8oc9ccccc9c78)cc6)c6ccc7ccccc7c6)cc5c4c3o2)cc1] 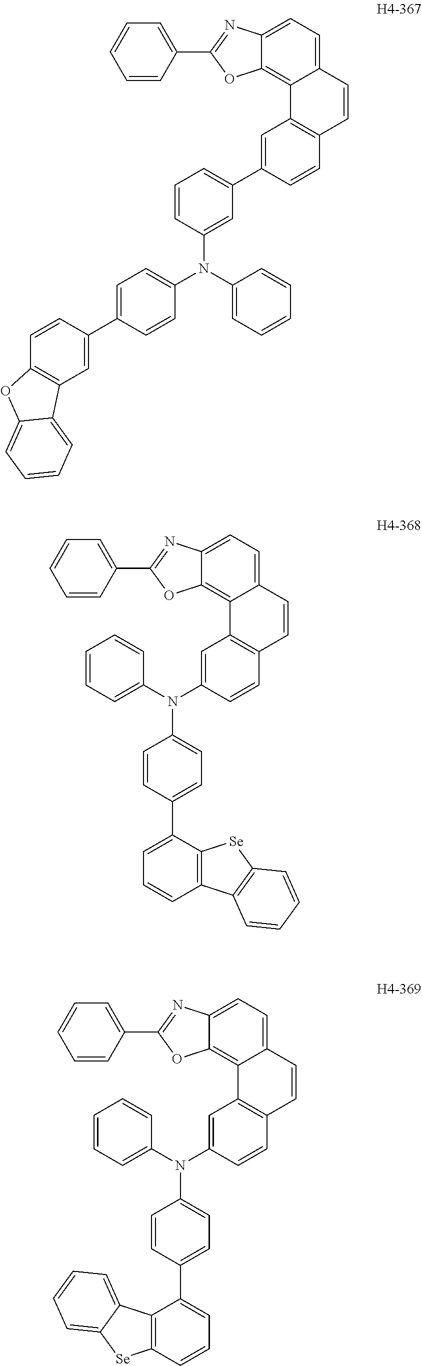 [SMILES: c1ccc(-c2nc3ccc4ccc5ccc(-c6cccc(N(c7ccccc7)c7ccc(-c8ccc9oc%10ccccc%10c9c8)cc7)c6)cc5c4c3o2)cc1 ; c1ccc(-c2nc3ccc4ccc5ccc(N(c6ccccc6)c6ccc(-c7cccc8c7[se]c7ccccc78)cc6)cc5c4c3o2)cc1 ; c1ccc(-c2nc3ccc4ccc5ccc(N(c6ccccc6)c6ccc(-c7cccc8[se]c9ccccc9c78)cc6)cc5c4c3o2)cc1] 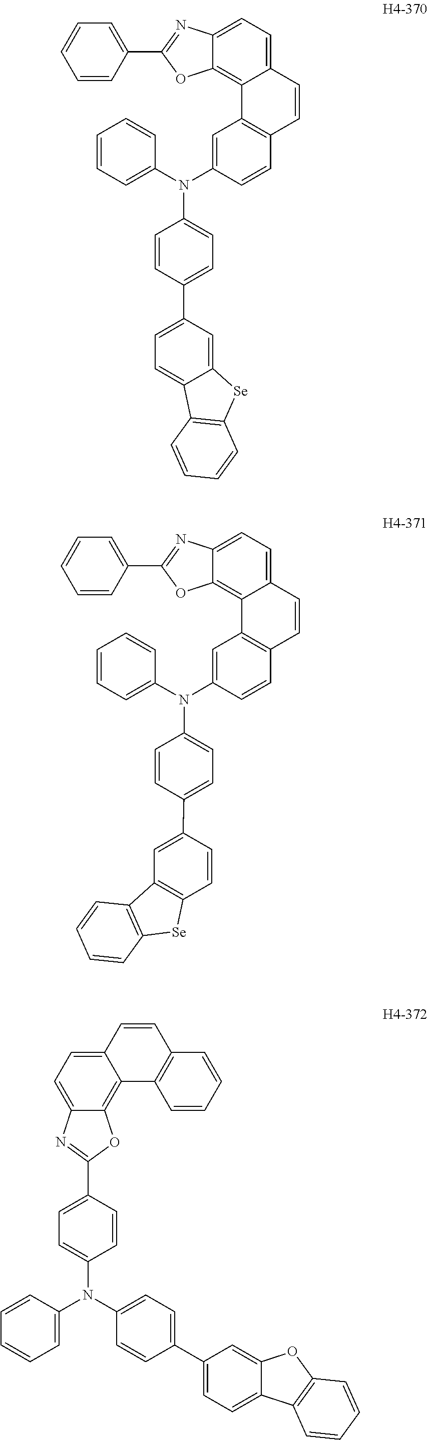 [SMILES: c1ccc(-c2nc3ccc4ccc5ccc(N(c6ccccc6)c6ccc(-c7ccc8c(c7)[se]c7ccccc78)cc6)cc5c4c3o2)cc1 ; c1ccc(-c2nc3ccc4ccc5ccc(N(c6ccccc6)c6ccc(-c7ccc8[se]c9ccccc9c8c7)cc6)cc5c4c3o2)cc1 ; c1ccc(N(c2ccc(-c3ccc4c(c3)oc3ccccc34)cc2)c2ccc(-c3nc4ccc5ccc6ccccc6c5c4o3)cc2)cc1] 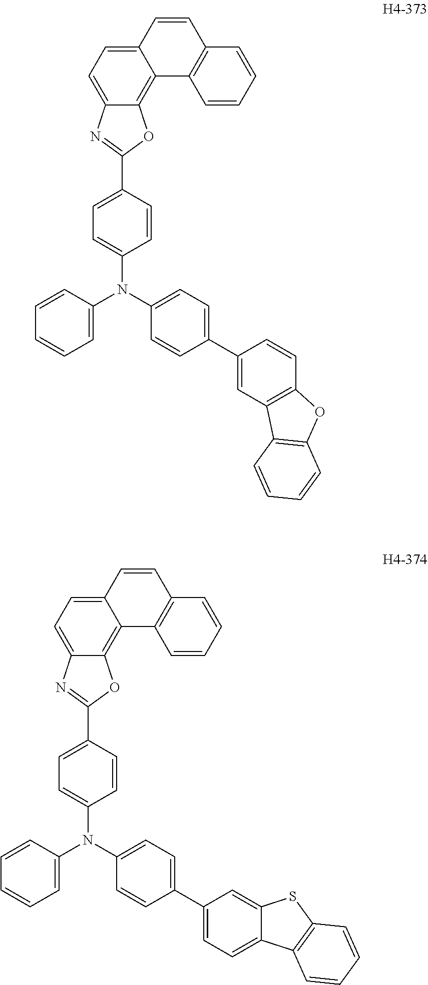 [SMILES: c1ccc(N(c2ccc(-c3ccc4oc5ccccc5c4c3)cc2)c2ccc(-c3nc4ccc5ccc6ccccc6c5c4o3)cc2)cc1 ; c1ccc(N(c2ccc(-c3ccc4c(c3)sc3ccccc34)cc2)c2ccc(-c3nc4ccc5ccc6ccccc6c5c4o3)cc2)cc1] 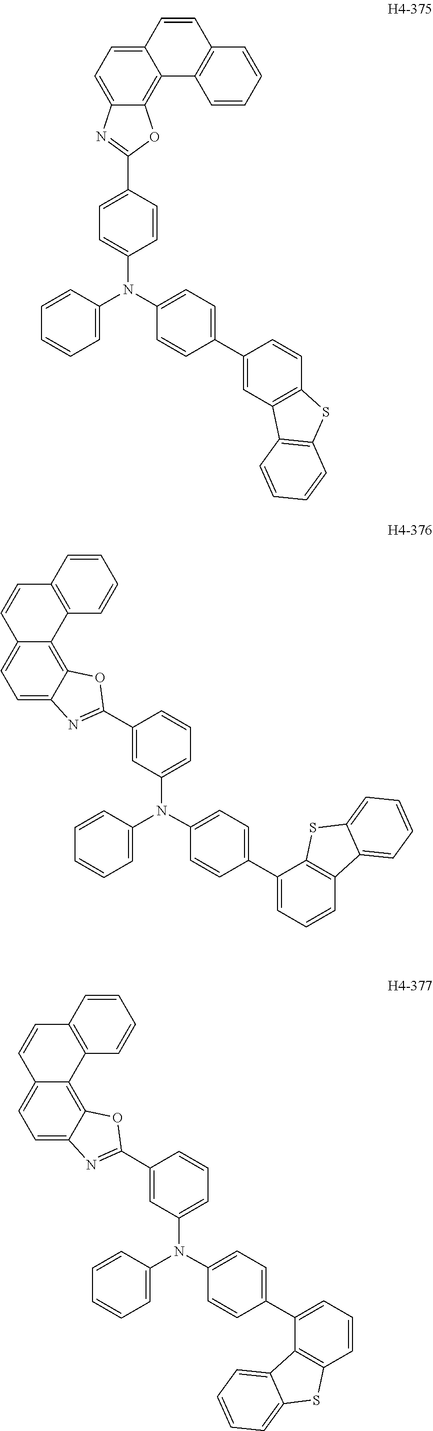 [SMILES: c1ccc(N(c2ccc(-c3ccc4sc5ccccc5c4c3)cc2)c2ccc(-c3nc4ccc5ccc6ccccc6c5c4o3)cc2)cc1 ; c1ccc(N(c2ccc(-c3cccc4c3sc3ccccc34)cc2)c2cccc(-c3nc4ccc5ccc6ccccc6c5c4o3)c2)cc1 ; c1ccc(N(c2ccc(-c3cccc4sc5ccccc5c34)cc2)c2cccc(-c3nc4ccc5ccc6ccccc6c5c4o3)c2)cc1] 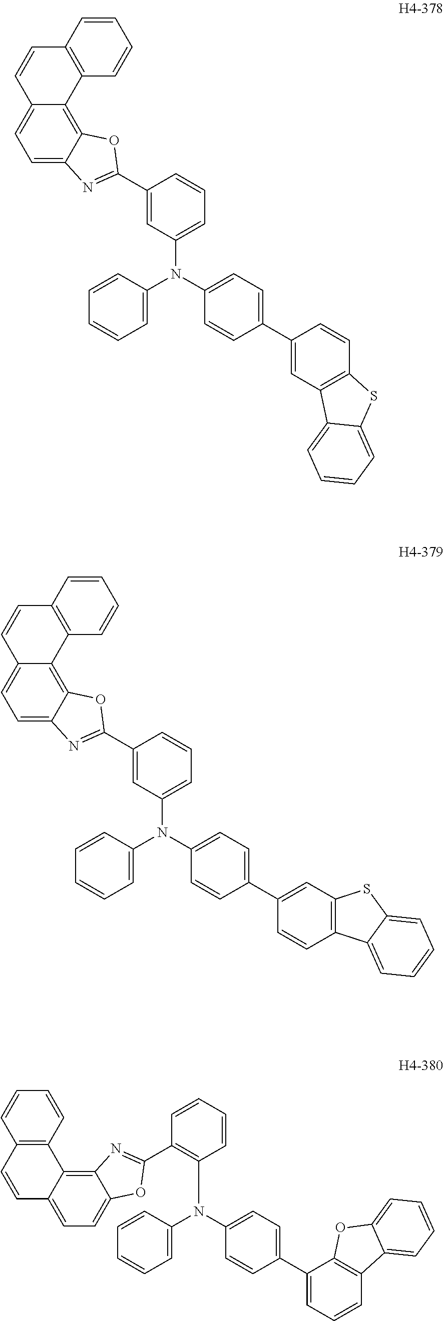 [SMILES: c1ccc(N(c2ccc(-c3ccc4sc5ccccc5c4c3)cc2)c2cccc(-c3nc4ccc5ccc6ccccc6c5c4o3)c2)cc1 ; c1ccc(N(c2ccc(-c3ccc4c(c3)sc3ccccc34)cc2)c2cccc(-c3nc4ccc5ccc6ccccc6c5c4o3)c2)cc1 ; c1ccc(N(c2ccc(-c3cccc4c3oc3ccccc34)cc2)c2ccccc2-c2nc3c(ccc4ccc5ccccc5c43)o2)cc1] 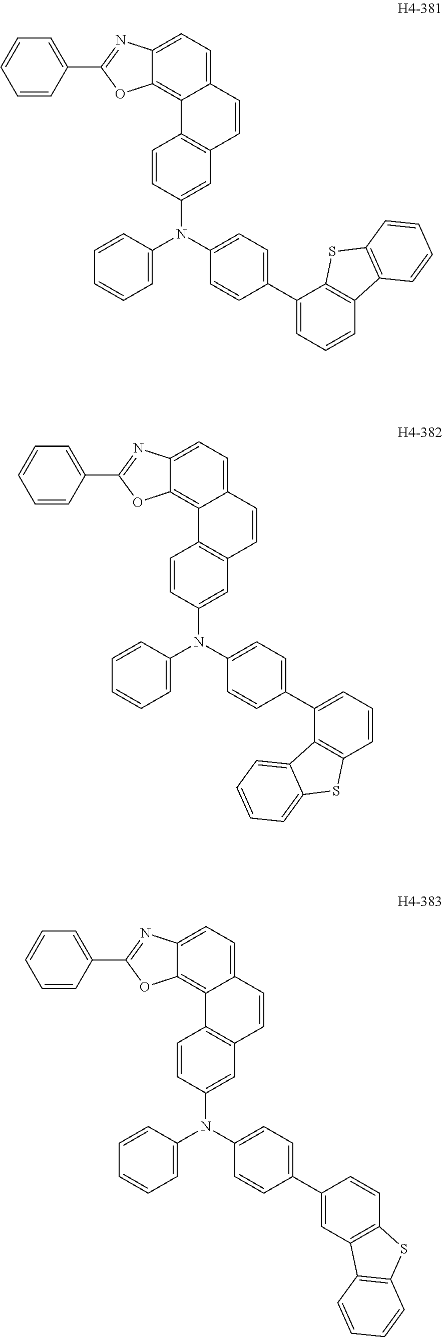 [SMILES: c1ccc(-c2nc3ccc4ccc5cc(N(c6ccccc6)c6ccc(-c7cccc8c7sc7ccccc78)cc6)ccc5c4c3o2)cc1 ; c1ccc(-c2nc3ccc4ccc5cc(N(c6ccccc6)c6ccc(-c7cccc8sc9ccccc9c78)cc6)ccc5c4c3o2)cc1 ; c1ccc(-c2nc3ccc4ccc5cc(N(c6ccccc6)c6ccc(-c7ccc8sc9ccccc9c8c7)cc6)ccc5c4c3o2)cc1] 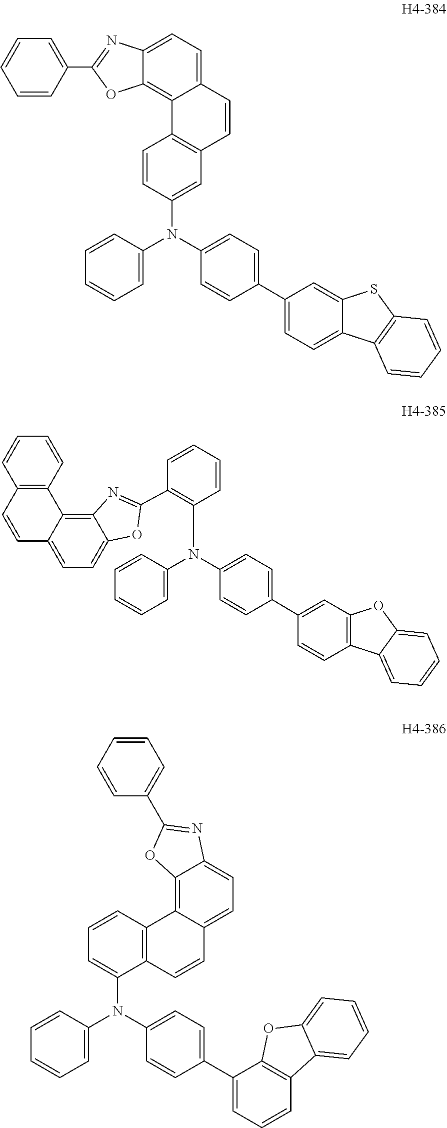 [SMILES: c1ccc(-c2nc3ccc4ccc5cc(N(c6ccccc6)c6ccc(-c7ccc8c(c7)sc7ccccc78)cc6)ccc5c4c3o2)cc1 ; c1ccc(N(c2ccc(-c3ccc4c(c3)oc3ccccc34)cc2)c2ccccc2-c2nc3c(ccc4ccc5ccccc5c43)o2)cc1 ; c1ccc(-c2nc3ccc4ccc5c(N(c6ccccc6)c6ccc(-c7cccc8c7oc7ccccc78)cc6)cccc5c4c3o2)cc1] 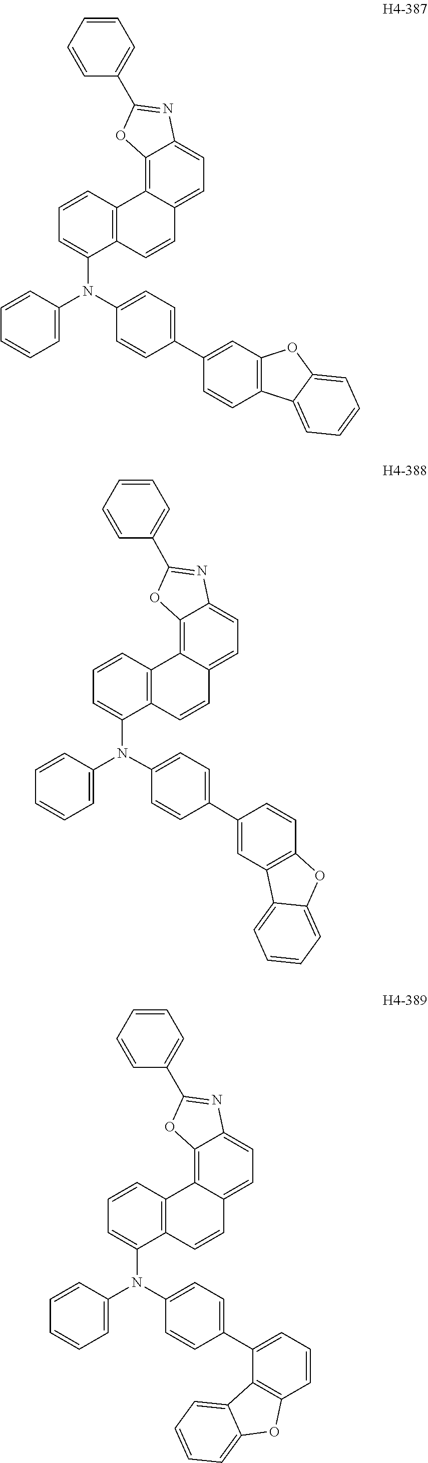 [SMILES: c1ccc(-c2nc3ccc4ccc5c(N(c6ccccc6)c6ccc(-c7ccc8c(c7)oc7ccccc78)cc6)cccc5c4c3o2)cc1 ; c1ccc(-c2nc3ccc4ccc5c(N(c6ccccc6)c6ccc(-c7ccc8oc9ccccc9c8c7)cc6)cccc5c4c3o2)cc1 ; c1ccc(-c2nc3ccc4ccc5c(N(c6ccccc6)c6ccc(-c7cccc8oc9ccccc9c78)cc6)cccc5c4c3o2)cc1] 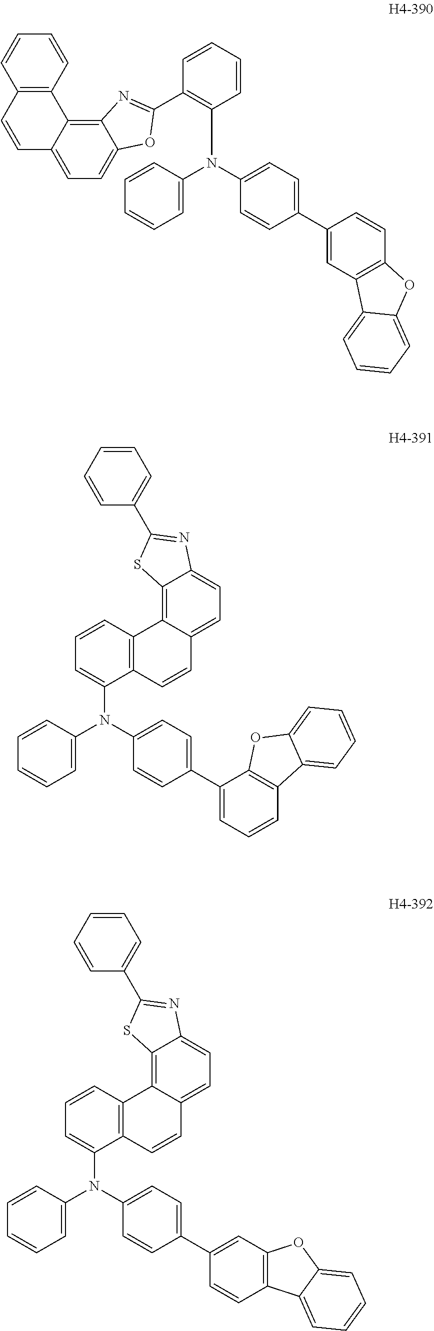 [SMILES: c1ccc(N(c2ccc(-c3ccc4oc5ccccc5c4c3)cc2)c2ccccc2-c2nc3c(ccc4ccc5ccccc5c43)o2)cc1 ; c1ccc(-c2nc3ccc4ccc5c(N(c6ccccc6)c6ccc(-c7cccc8c7oc7ccccc78)cc6)cccc5c4c3s2)cc1 ; c1ccc(-c2nc3ccc4ccc5c(N(c6ccccc6)c6ccc(-c7ccc8c(c7)oc7ccccc78)cc6)cccc5c4c3s2)cc1] 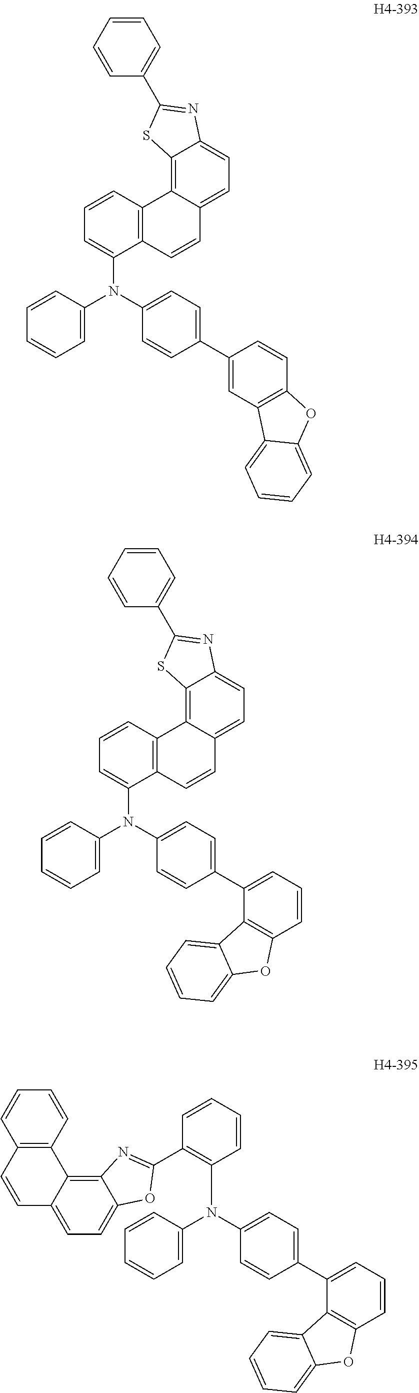 [SMILES: c1ccc(-c2nc3ccc4ccc5c(N(c6ccccc6)c6ccc(-c7ccc8oc9ccccc9c8c7)cc6)cccc5c4c3s2)cc1 ; c1ccc(-c2nc3ccc4ccc5c(N(c6ccccc6)c6ccc(-c7cccc8oc9ccccc9c78)cc6)cccc5c4c3s2)cc1 ; c1ccc(N(c2ccc(-c3cccc4oc5ccccc5c34)cc2)c2ccccc2-c2nc3c(ccc4ccc5ccccc5c43)o2)cc1] 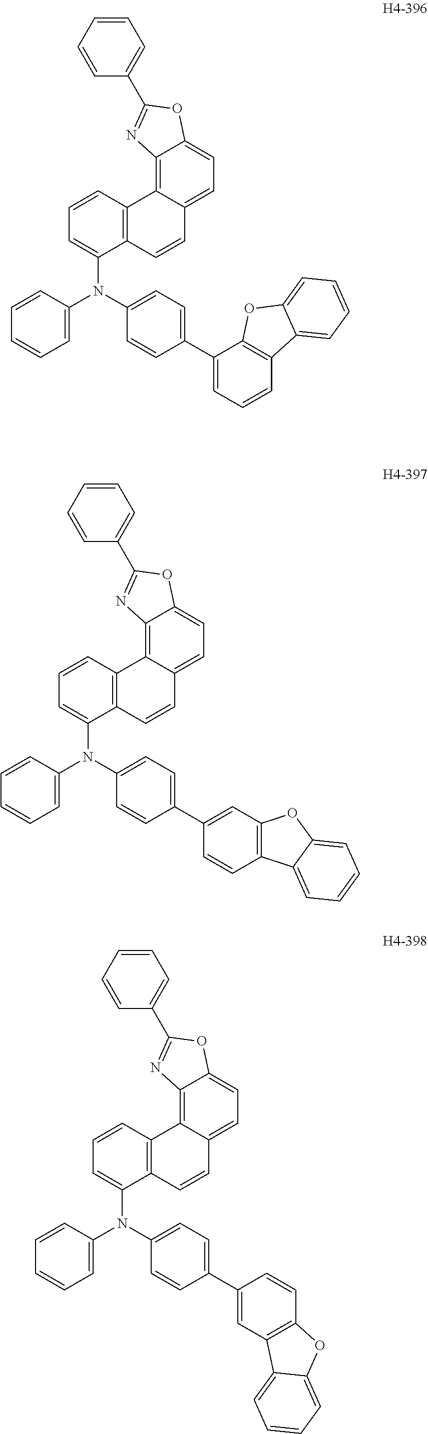 [SMILES: c1ccc(-c2nc3c(ccc4ccc5c(N(c6ccccc6)c6ccc(-c7cccc8c7oc7ccccc78)cc6)cccc5c43)o2)cc1 ; c1ccc(-c2nc3c(ccc4ccc5c(N(c6ccccc6)c6ccc(-c7ccc8c(c7)oc7ccccc78)cc6)cccc5c43)o2)cc1 ; c1ccc(-c2nc3c(ccc4ccc5c(N(c6ccccc6)c6ccc(-c7ccc8oc9ccccc9c8c7)cc6)cccc5c43)o2)cc1] 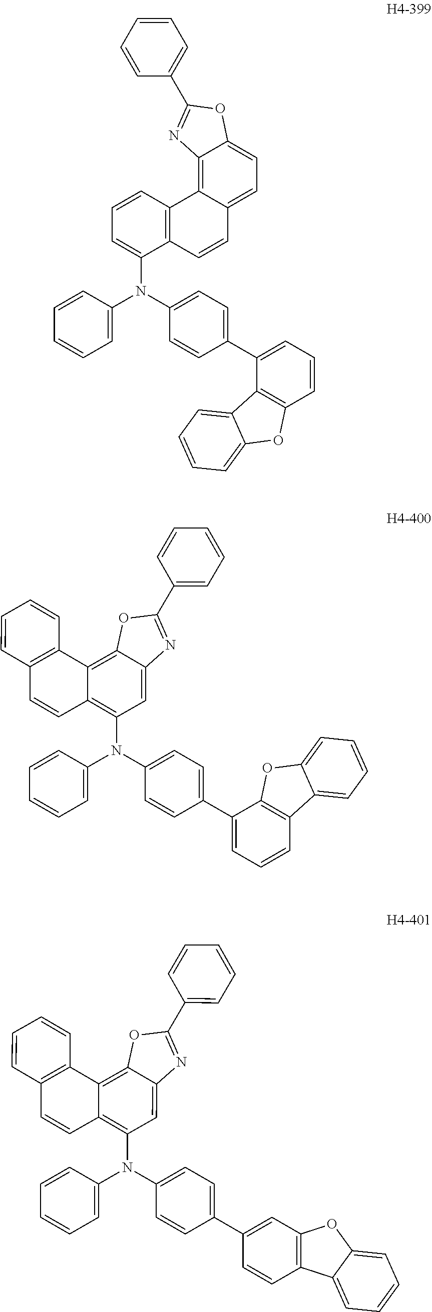 [SMILES: c1ccc(-c2nc3c(ccc4ccc5c(N(c6ccccc6)c6ccc(-c7cccc8oc9ccccc9c78)cc6)cccc5c43)o2)cc1 ; c1ccc(-c2nc3cc(N(c4ccccc4)c4ccc(-c5cccc6c5oc5ccccc56)cc4)c4ccc5ccccc5c4c3o2)cc1 ; c1ccc(-c2nc3cc(N(c4ccccc4)c4ccc(-c5ccc6c(c5)oc5ccccc56)cc4)c4ccc5ccccc5c4c3o2)cc1] 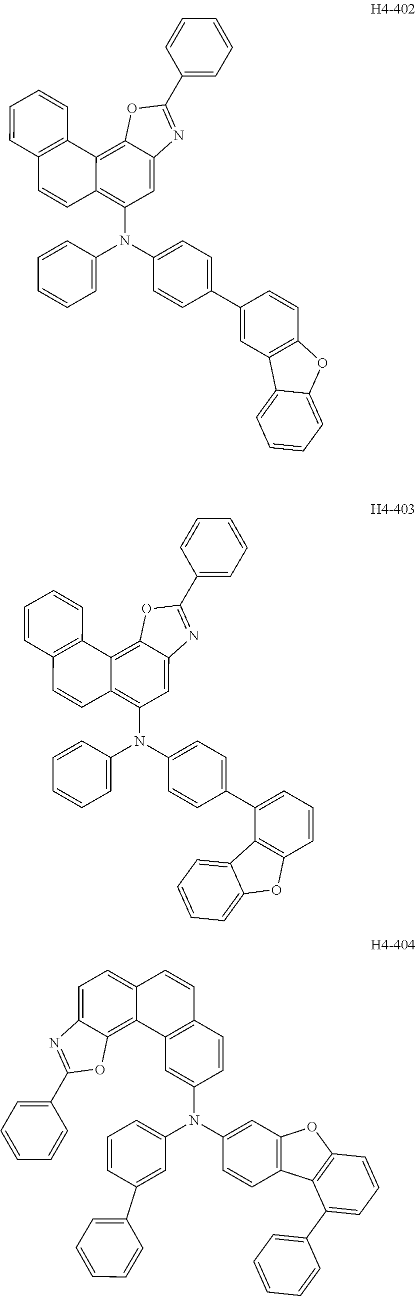 [SMILES: c1ccc(-c2nc3cc(N(c4ccccc4)c4ccc(-c5ccc6oc7ccccc7c6c5)cc4)c4ccc5ccccc5c4c3o2)cc1 ; c1ccc(-c2nc3cc(N(c4ccccc4)c4ccc(-c5cccc6oc7ccccc7c56)cc4)c4ccc5ccccc5c4c3o2)cc1 ; c1ccc(-c2cccc(N(c3ccc4c(c3)oc3cccc(-c5ccccc5)c34)c3ccc4ccc5ccc6nc(-c7ccccc7)oc6c5c4c3)c2)cc1] 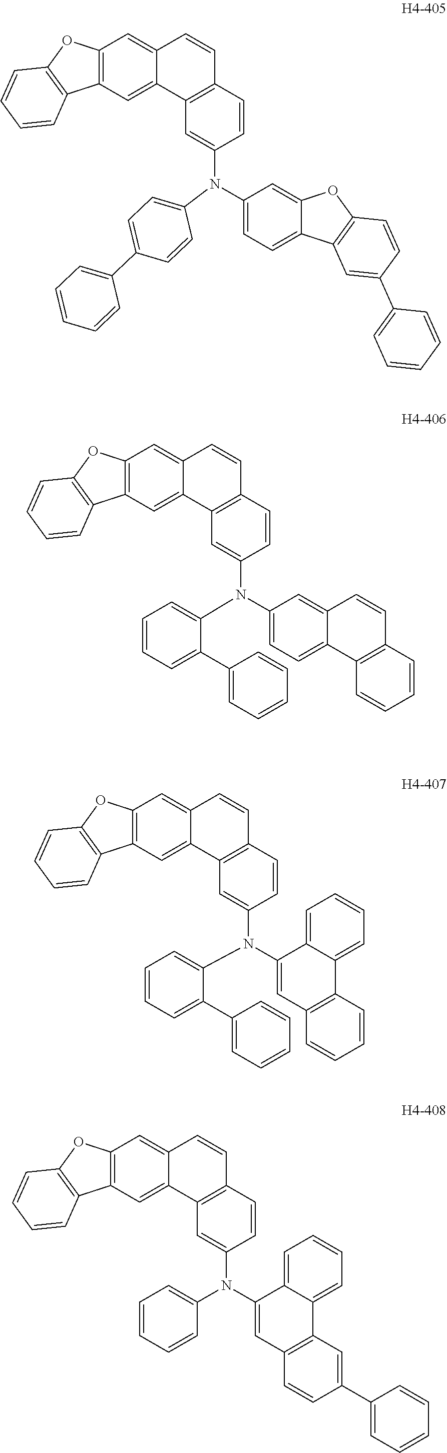 [SMILES: c1ccc(-c2ccc(N(c3ccc4c(c3)oc3ccc(-c5ccccc5)cc34)c3ccc4ccc5cc6oc7ccccc7c6cc5c4c3)cc2)cc1 ; c1ccc(-c2ccccc2N(c2ccc3c(ccc4ccccc43)c2)c2ccc3ccc4cc5oc6ccccc6c5cc4c3c2)cc1 ; c1ccc(-c2ccccc2N(c2ccc3ccc4cc5oc6ccccc6c5cc4c3c2)c2cc3ccccc3c3ccccc23)cc1 ; c1ccc(-c2ccc3cc(N(c4ccccc4)c4ccc5ccc6cc7oc8ccccc8c7cc6c5c4)c4ccccc4c3c2)cc1] 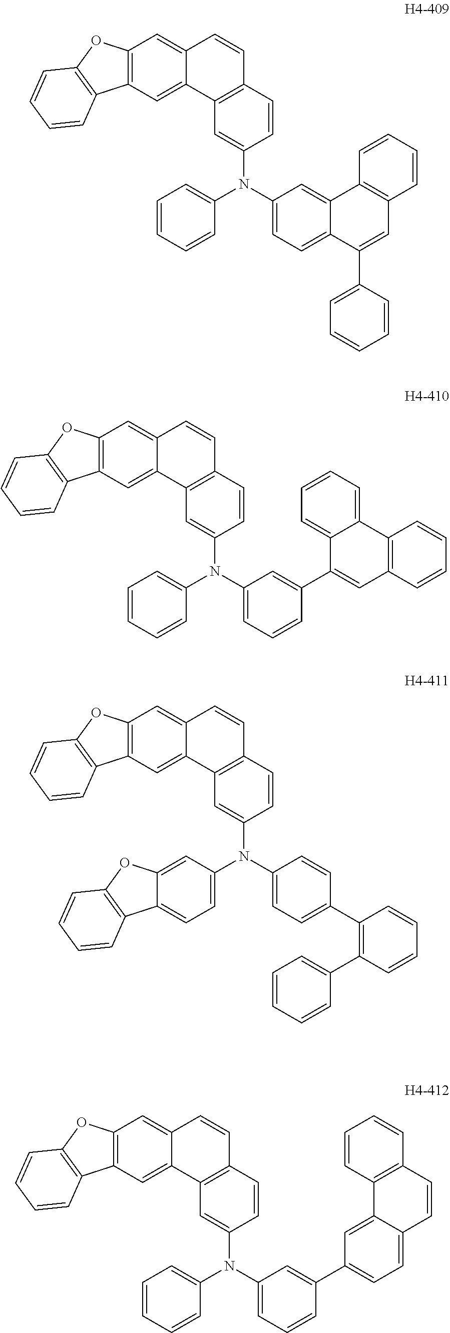 [SMILES: c1ccc(-c2cc3ccccc3c3cc(N(c4ccccc4)c4ccc5ccc6cc7oc8ccccc8c7cc6c5c4)ccc23)cc1 ; c1ccc(N(c2cccc(-c3cc4ccccc4c4ccccc34)c2)c2ccc3ccc4cc5oc6ccccc6c5cc4c3c2)cc1 ; c1ccc(-c2ccccc2-c2ccc(N(c3ccc4c(c3)oc3ccccc34)c3ccc4ccc5cc6oc7ccccc7c6cc5c4c3)cc2)cc1 ; c1ccc(N(c2cccc(-c3ccc4ccc5ccccc5c4c3)c2)c2ccc3ccc4cc5oc6ccccc6c5cc4c3c2)cc1] 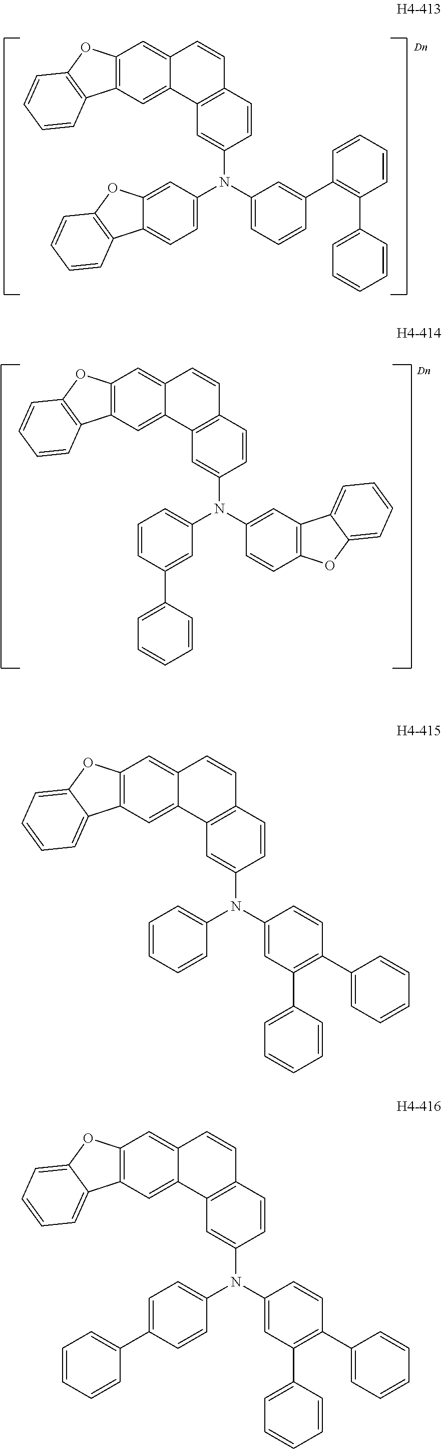 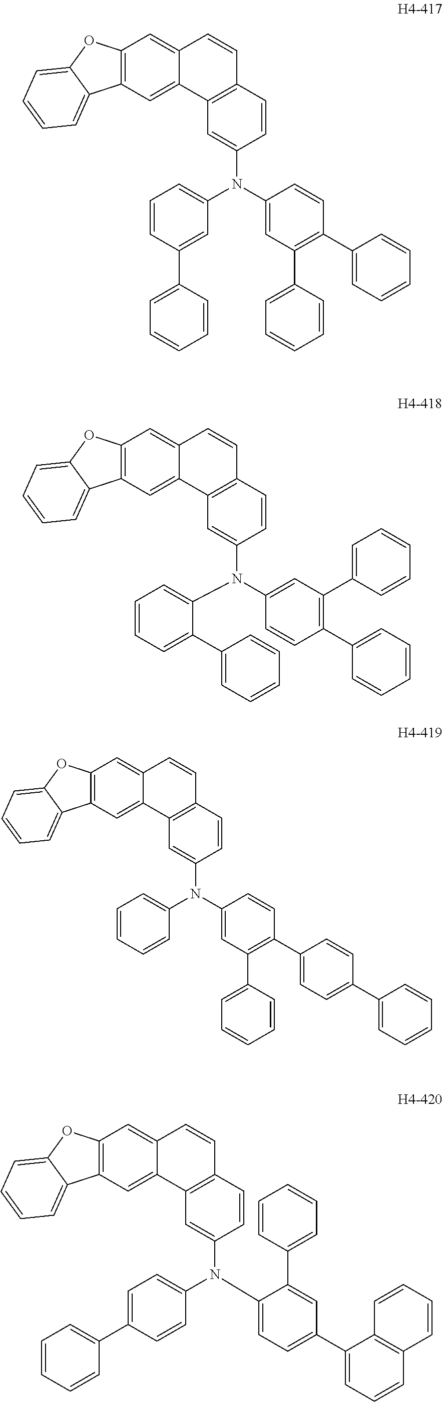 [SMILES: c1ccc(-c2cccc(N(c3ccc(-c4ccccc4)c(-c4ccccc4)c3)c3ccc4ccc5cc6oc7ccccc7c6cc5c4c3)c2)cc1 ; c1ccc(-c2ccc(N(c3ccc4ccc5cc6oc7ccccc7c6cc5c4c3)c3ccccc3-c3ccccc3)cc2-c2ccccc2)cc1 ; c1ccc(-c2ccc(-c3ccc(N(c4ccccc4)c4ccc5ccc6cc7oc8ccccc8c7cc6c5c4)cc3-c3ccccc3)cc2)cc1 ; c1ccc(-c2ccc(N(c3ccc4ccc5cc6oc7ccccc7c6cc5c4c3)c3ccc(-c4cccc5ccccc45)cc3-c3ccccc3)cc2)cc1] 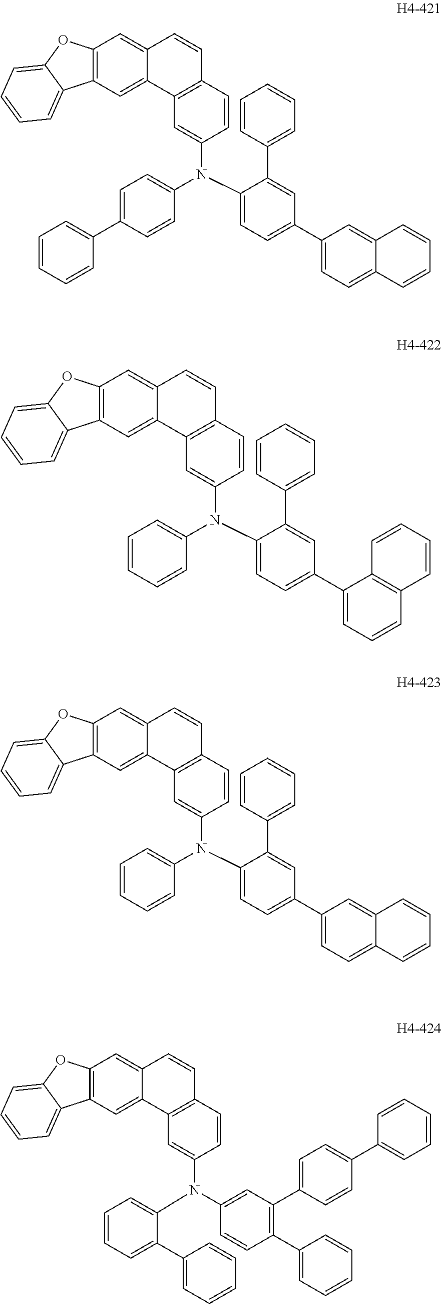 [SMILES: c1ccc(-c2ccc(N(c3ccc4ccc5cc6oc7ccccc7c6cc5c4c3)c3ccc(-c4ccc5ccccc5c4)cc3-c3ccccc3)cc2)cc1 ; c1ccc(-c2cc(-c3cccc4ccccc34)ccc2N(c2ccccc2)c2ccc3ccc4cc5oc6ccccc6c5cc4c3c2)cc1 ; c1ccc(-c2cc(-c3ccc4ccccc4c3)ccc2N(c2ccccc2)c2ccc3ccc4cc5oc6ccccc6c5cc4c3c2)cc1 ; c1ccc(-c2ccc(-c3cc(N(c4ccc5ccc6cc7oc8ccccc8c7cc6c5c4)c4ccccc4-c4ccccc4)ccc3-c3ccccc3)cc2)cc1] 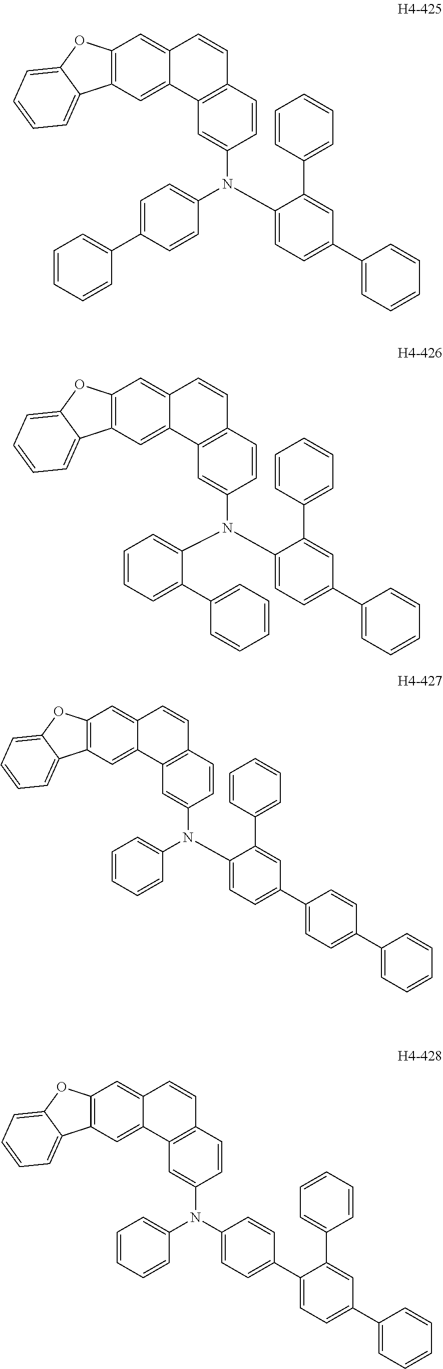 [SMILES: c1ccc(-c2ccc(N(c3ccc4ccc5cc6oc7ccccc7c6cc5c4c3)c3ccc(-c4ccccc4)cc3-c3ccccc3)cc2)cc1 ; c1ccc(-c2ccc(N(c3ccc4ccc5cc6oc7ccccc7c6cc5c4c3)c3ccccc3-c3ccccc3)c(-c3ccccc3)c2)cc1 ; c1ccc(-c2ccc(-c3ccc(N(c4ccccc4)c4ccc5ccc6cc7oc8ccccc8c7cc6c5c4)c(-c4ccccc4)c3)cc2)cc1 ; c1ccc(-c2ccc(-c3ccc(N(c4ccccc4)c4ccc5ccc6cc7oc8ccccc8c7cc6c5c4)cc3)c(-c3ccccc3)c2)cc1] 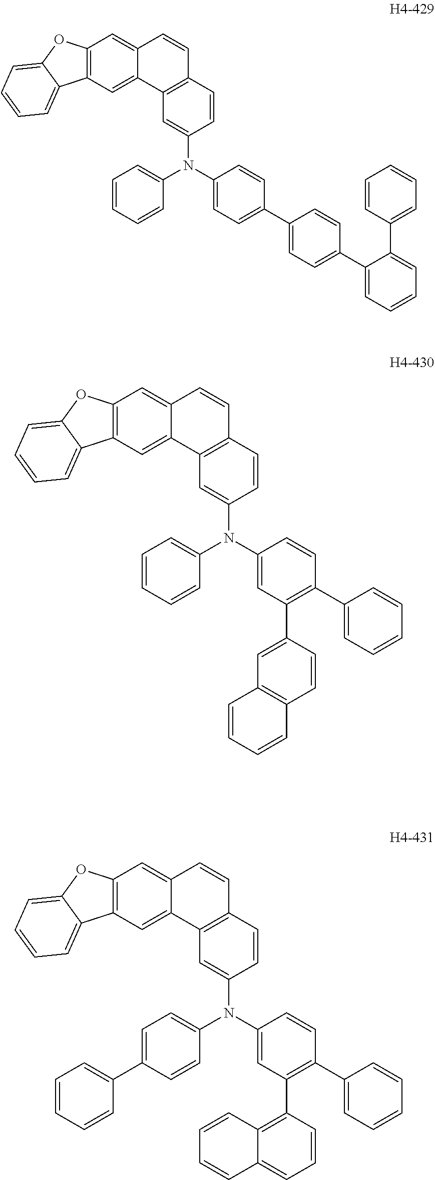 [SMILES: c1ccc(-c2ccccc2-c2ccc(-c3ccc(N(c4ccccc4)c4ccc5ccc6cc7oc8ccccc8c7cc6c5c4)cc3)cc2)cc1 ; c1ccc(-c2ccc(N(c3ccccc3)c3ccc4ccc5cc6oc7ccccc7c6cc5c4c3)cc2-c2ccc3ccccc3c2)cc1 ; c1ccc(-c2ccc(N(c3ccc(-c4ccccc4)c(-c4cccc5ccccc45)c3)c3ccc4ccc5cc6oc7ccccc7c6cc5c4c3)cc2)cc1] 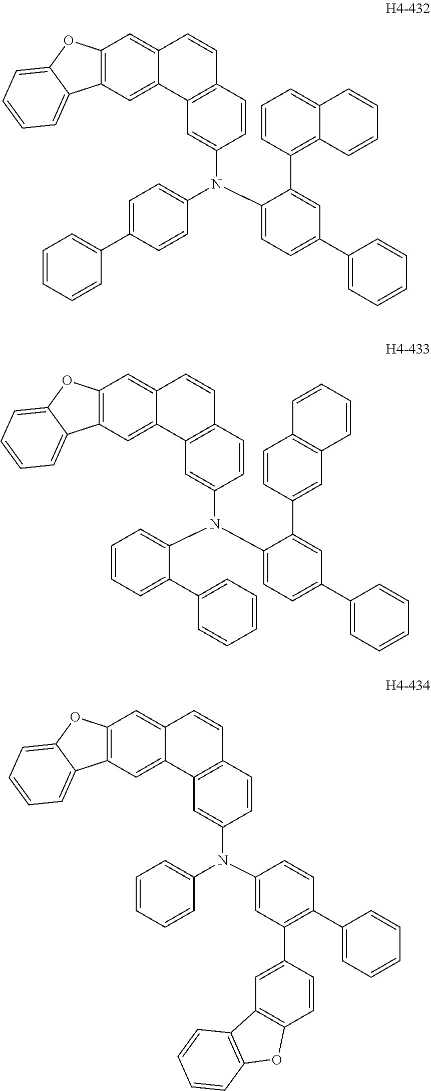 [SMILES: c1ccc(-c2ccc(N(c3ccc4ccc5cc6oc7ccccc7c6cc5c4c3)c3ccc(-c4ccccc4)cc3-c3cccc4ccccc34)cc2)cc1 ; c1ccc(-c2ccc(N(c3ccc4ccc5cc6oc7ccccc7c6cc5c4c3)c3ccccc3-c3ccccc3)c(-c3ccc4ccccc4c3)c2)cc1 ; c1ccc(-c2ccc(N(c3ccccc3)c3ccc4ccc5cc6oc7ccccc7c6cc5c4c3)cc2-c2ccc3oc4ccccc4c3c2)cc1] 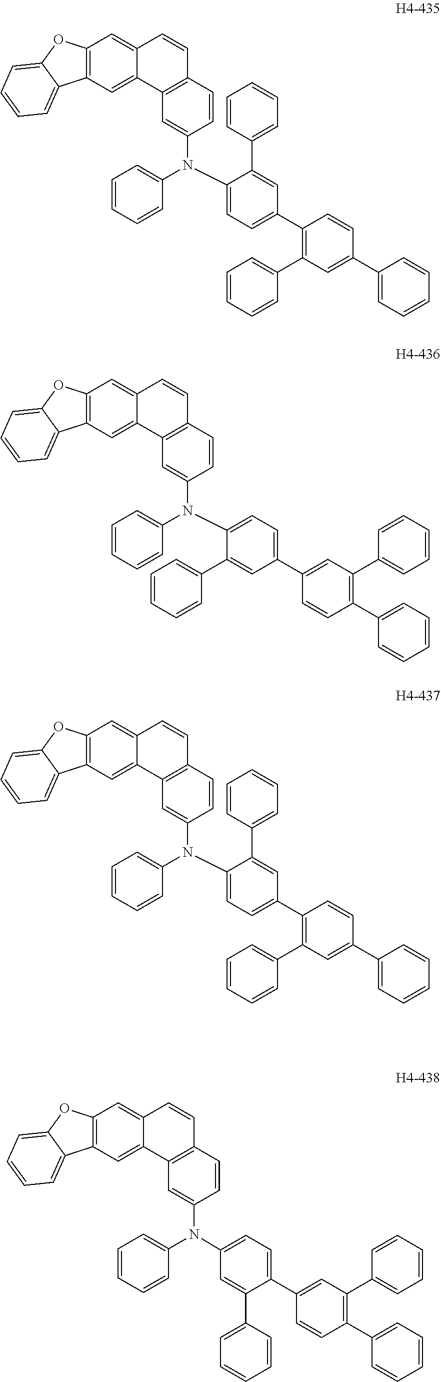 [SMILES: c1ccc(-c2ccc(-c3ccc(N(c4ccccc4)c4ccc5ccc6cc7oc8ccccc8c7cc6c5c4)c(-c4ccccc4)c3)c(-c3ccccc3)c2)cc1 ; c1ccc(-c2ccc(-c3ccc(N(c4ccccc4)c4ccc5ccc6cc7oc8ccccc8c7cc6c5c4)c(-c4ccccc4)c3)cc2-c2ccccc2)cc1 ; c1ccc(-c2ccc(-c3ccc(N(c4ccccc4)c4ccc5ccc6cc7oc8ccccc8c7cc6c5c4)c(-c4ccccc4)c3)c(-c3ccccc3)c2)cc1 ; c1ccc(-c2ccc(-c3ccc(N(c4ccccc4)c4ccc5ccc6cc7oc8ccccc8c7cc6c5c4)cc3-c3ccccc3)cc2-c2ccccc2)cc1] 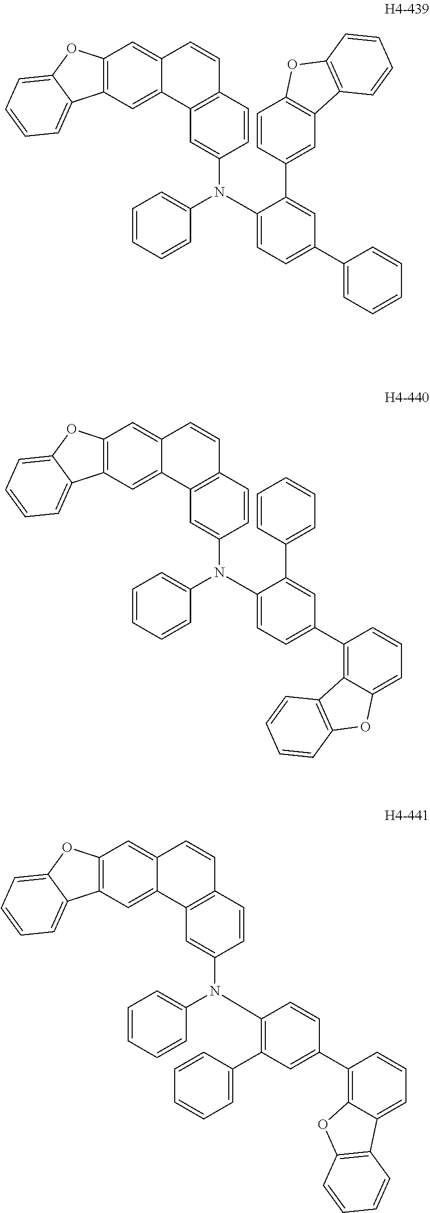 [SMILES: c1ccc(-c2ccc(N(c3ccccc3)c3ccc4ccc5cc6oc7ccccc7c6cc5c4c3)c(-c3ccc4oc5ccccc5c4c3)c2)cc1 ; c1ccc(-c2cc(-c3cccc4oc5ccccc5c34)ccc2N(c2ccccc2)c2ccc3ccc4cc5oc6ccccc6c5cc4c3c2)cc1 ; c1ccc(-c2cc(-c3cccc4c3oc3ccccc34)ccc2N(c2ccccc2)c2ccc3ccc4cc5oc6ccccc6c5cc4c3c2)cc1] 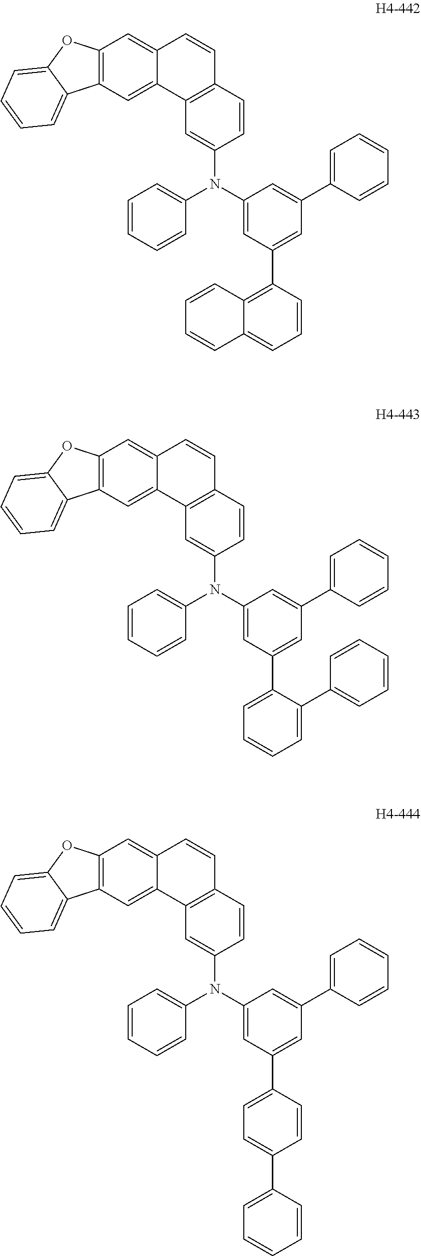 [SMILES: c1ccc(-c2cc(-c3cccc4ccccc34)cc(N(c3ccccc3)c3ccc4ccc5cc6oc7ccccc7c6cc5c4c3)c2)cc1 ; c1ccc(-c2cc(-c3ccccc3-c3ccccc3)cc(N(c3ccccc3)c3ccc4ccc5cc6oc7ccccc7c6cc5c4c3)c2)cc1 ; c1ccc(-c2ccc(-c3cc(-c4ccccc4)cc(N(c4ccccc4)c4ccc5ccc6cc7oc8ccccc8c7cc6c5c4)c3)cc2)cc1] 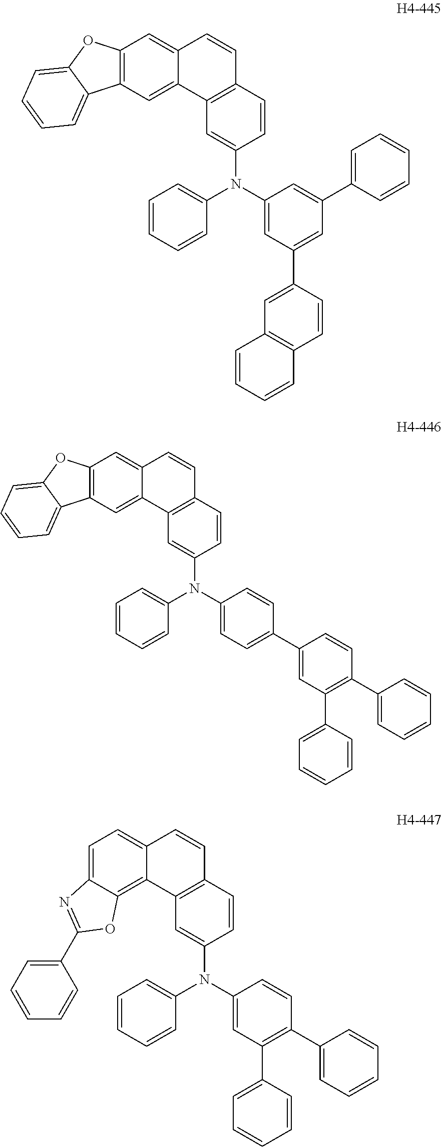 [SMILES: c1ccc(-c2cc(-c3ccc4ccccc4c3)cc(N(c3ccccc3)c3ccc4ccc5cc6oc7ccccc7c6cc5c4c3)c2)cc1 ; c1ccc(-c2ccc(-c3ccc(N(c4ccccc4)c4ccc5ccc6cc7oc8ccccc8c7cc6c5c4)cc3)cc2-c2ccccc2)cc1 ; c1ccc(-c2nc3ccc4ccc5ccc(N(c6ccccc6)c6ccc(-c7ccccc7)c(-c7ccccc7)c6)cc5c4c3o2)cc1] 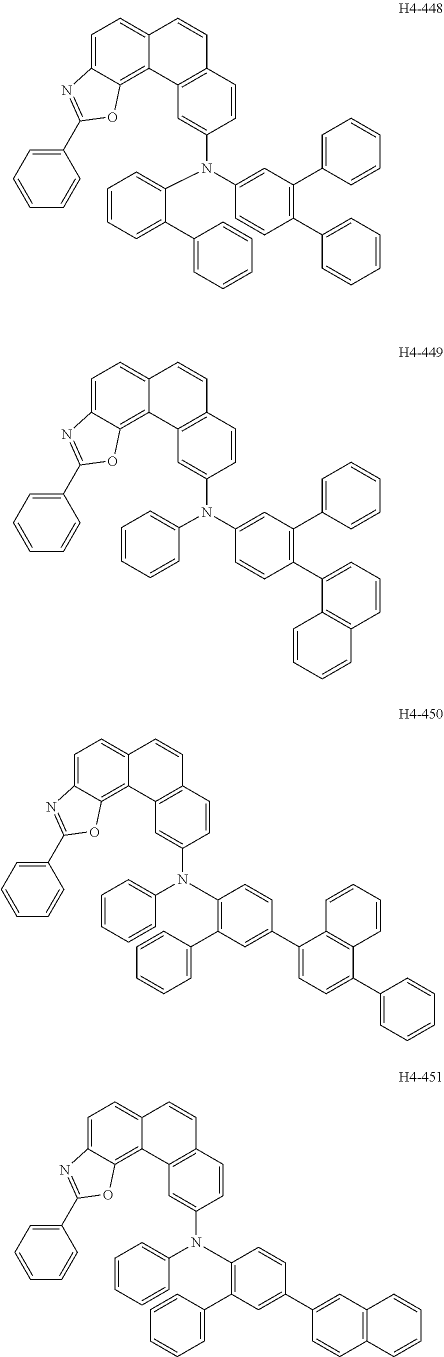 [SMILES: c1ccc(-c2nc3ccc4ccc5ccc(N(c6ccc(-c7ccccc7)c(-c7ccccc7)c6)c6ccccc6-c6ccccc6)cc5c4c3o2)cc1 ; c1ccc(-c2nc3ccc4ccc5ccc(N(c6ccccc6)c6ccc(-c7cccc8ccccc78)c(-c7ccccc7)c6)cc5c4c3o2)cc1 ; c1ccc(-c2nc3ccc4ccc5ccc(N(c6ccccc6)c6ccc(-c7ccc(-c8ccccc8)c8ccccc78)cc6-c6ccccc6)cc5c4c3o2)cc1 ; c1ccc(-c2nc3ccc4ccc5ccc(N(c6ccccc6)c6ccc(-c7ccc8ccccc8c7)cc6-c6ccccc6)cc5c4c3o2)cc1] 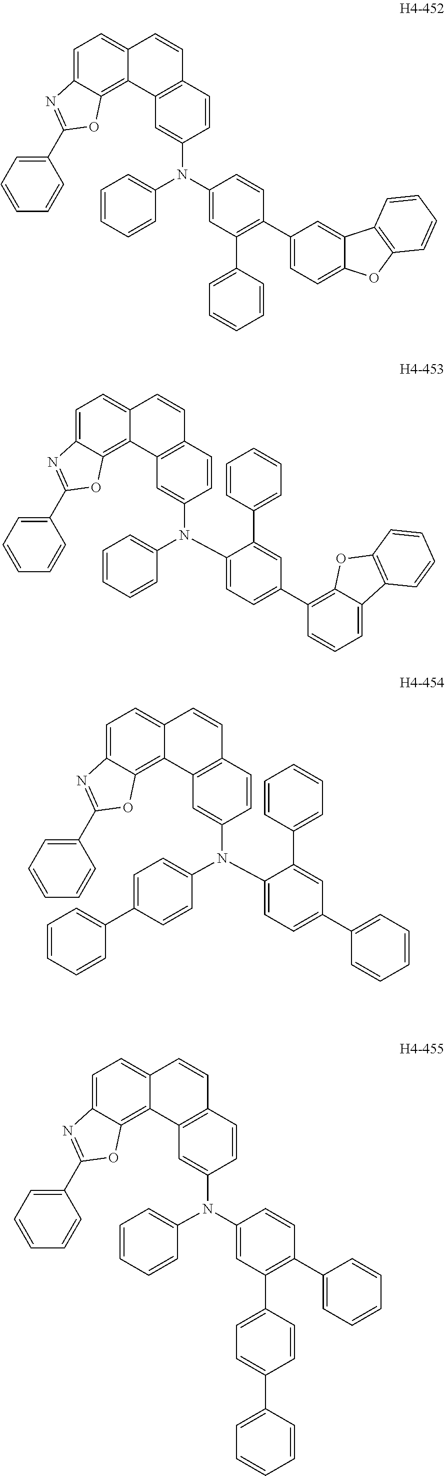 [SMILES: c1ccc(-c2nc3ccc4ccc5ccc(N(c6ccccc6)c6ccc(-c7ccc8oc9ccccc9c8c7)c(-c7ccccc7)c6)cc5c4c3o2)cc1 ; c1ccc(-c2nc3ccc4ccc5ccc(N(c6ccccc6)c6ccc(-c7cccc8c7oc7ccccc78)cc6-c6ccccc6)cc5c4c3o2)cc1 ; c1ccc(-c2ccc(N(c3ccc4ccc5ccc6nc(-c7ccccc7)oc6c5c4c3)c3ccc(-c4ccccc4)cc3-c3ccccc3)cc2)cc1 ; c1ccc(-c2ccc(-c3cc(N(c4ccccc4)c4ccc5ccc6ccc7nc(-c8ccccc8)oc7c6c5c4)ccc3-c3ccccc3)cc2)cc1] 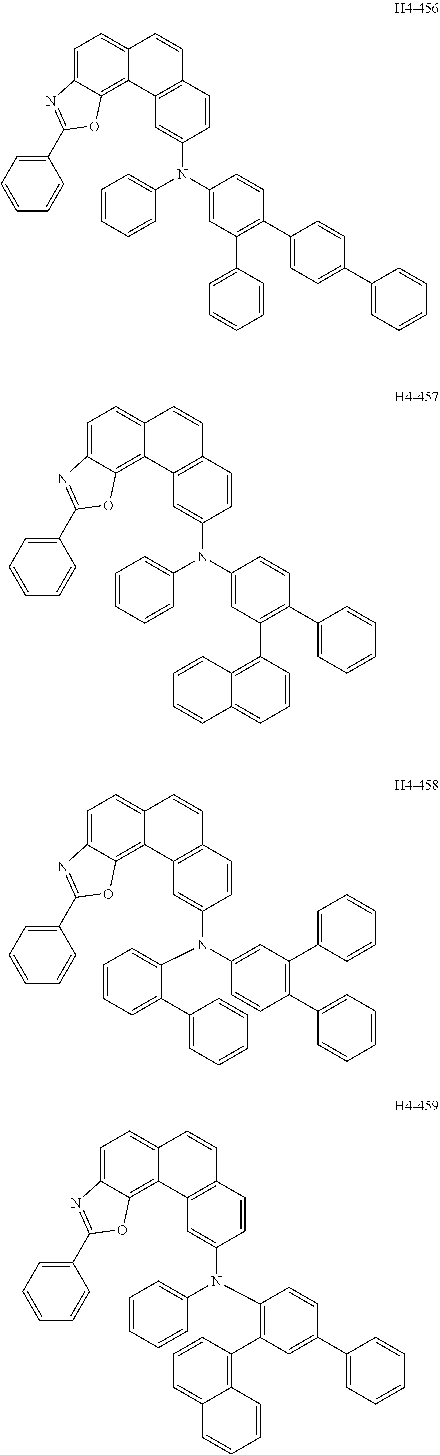 [SMILES: c1ccc(-c2ccc(-c3ccc(N(c4ccccc4)c4ccc5ccc6ccc7nc(-c8ccccc8)oc7c6c5c4)cc3-c3ccccc3)cc2)cc1 ; c1ccc(-c2nc3ccc4ccc5ccc(N(c6ccccc6)c6ccc(-c7ccccc7)c(-c7cccc8ccccc78)c6)cc5c4c3o2)cc1 ; c1ccc(-c2nc3ccc4ccc5ccc(N(c6ccc(-c7ccccc7)c(-c7ccccc7)c6)c6ccccc6-c6ccccc6)cc5c4c3o2)cc1 ; c1ccc(-c2ccc(N(c3ccccc3)c3ccc4ccc5ccc6nc(-c7ccccc7)oc6c5c4c3)c(-c3cccc4ccccc34)c2)cc1] 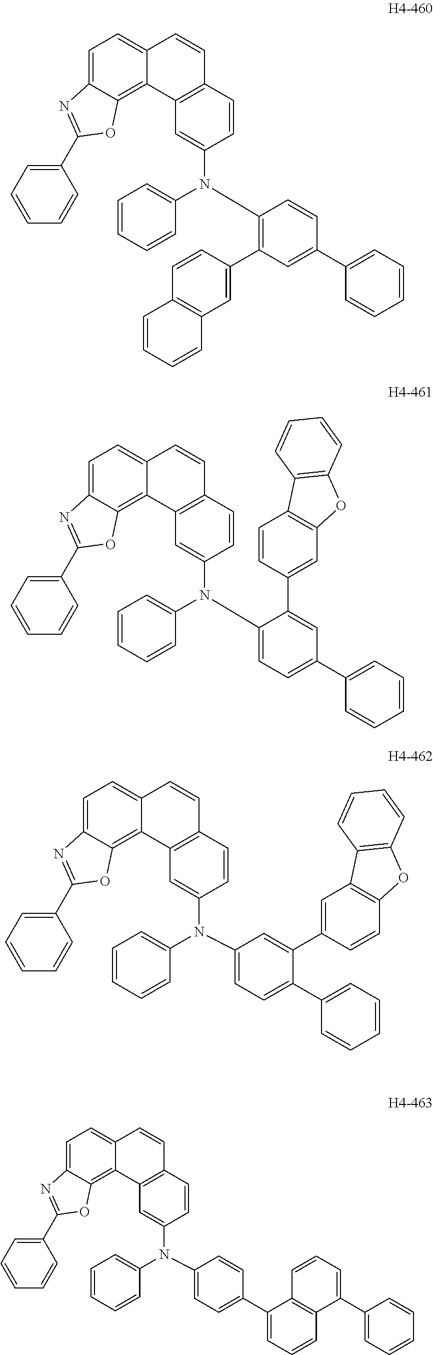 [SMILES: c1ccc(-c2ccc(N(c3ccccc3)c3ccc4ccc5ccc6nc(-c7ccccc7)oc6c5c4c3)c(-c3ccc4ccccc4c3)c2)cc1 ; c1ccc(-c2ccc(N(c3ccccc3)c3ccc4ccc5ccc6nc(-c7ccccc7)oc6c5c4c3)c(-c3ccc4c(c3)oc3ccccc34)c2)cc1 ; c1ccc(-c2nc3ccc4ccc5ccc(N(c6ccccc6)c6ccc(-c7ccccc7)c(-c7ccc8oc9ccccc9c8c7)c6)cc5c4c3o2)cc1 ; c1ccc(-c2nc3ccc4ccc5ccc(N(c6ccccc6)c6ccc(-c7cccc8c(-c9ccccc9)cccc78)cc6)cc5c4c3o2)cc1] 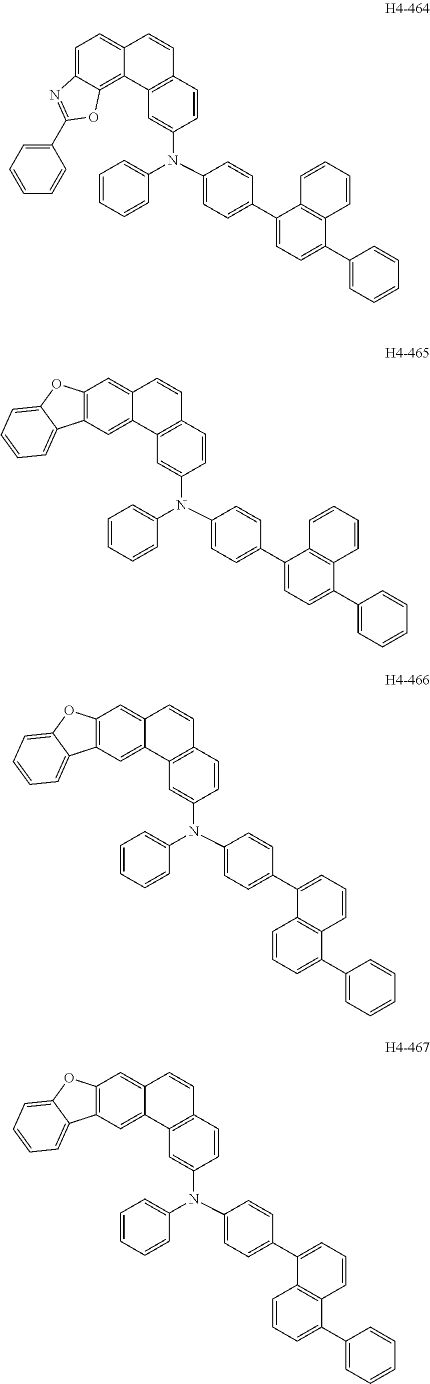 [SMILES: c1ccc(-c2nc3ccc4ccc5ccc(N(c6ccccc6)c6ccc(-c7ccc(-c8ccccc8)c8ccccc78)cc6)cc5c4c3o2)cc1 ; c1ccc(-c2ccc(-c3ccc(N(c4ccccc4)c4ccc5ccc6cc7oc8ccccc8c7cc6c5c4)cc3)c3ccccc23)cc1 ; c1ccc(-c2cccc3c(-c4ccc(N(c5ccccc5)c5ccc6ccc7cc8oc9ccccc9c8cc7c6c5)cc4)cccc23)cc1 ; c1ccc(-c2cccc3c(-c4ccc(N(c5ccccc5)c5ccc6ccc7cc8oc9ccccc9c8cc7c6c5)cc4)cccc23)cc1] 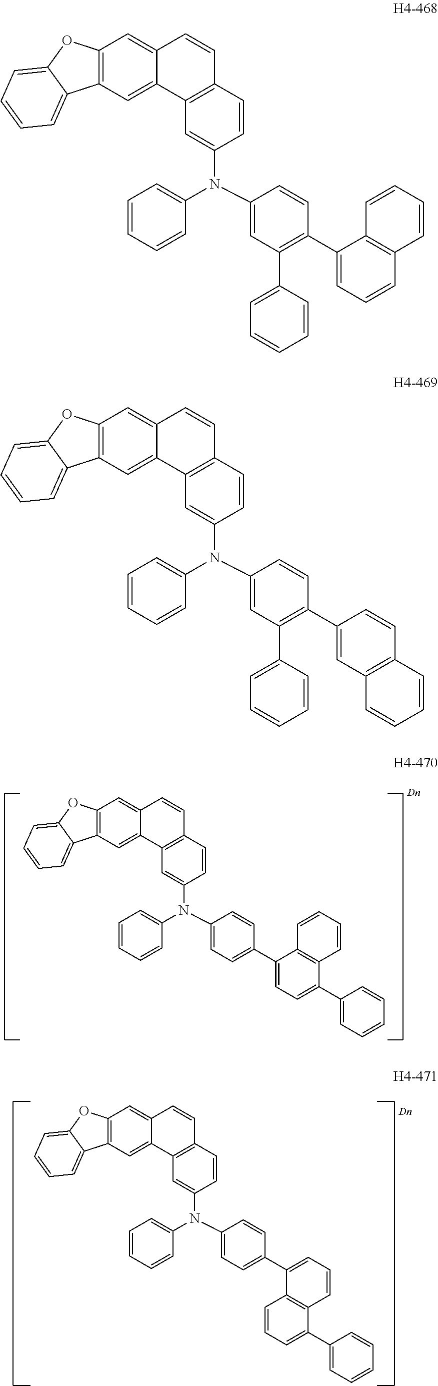 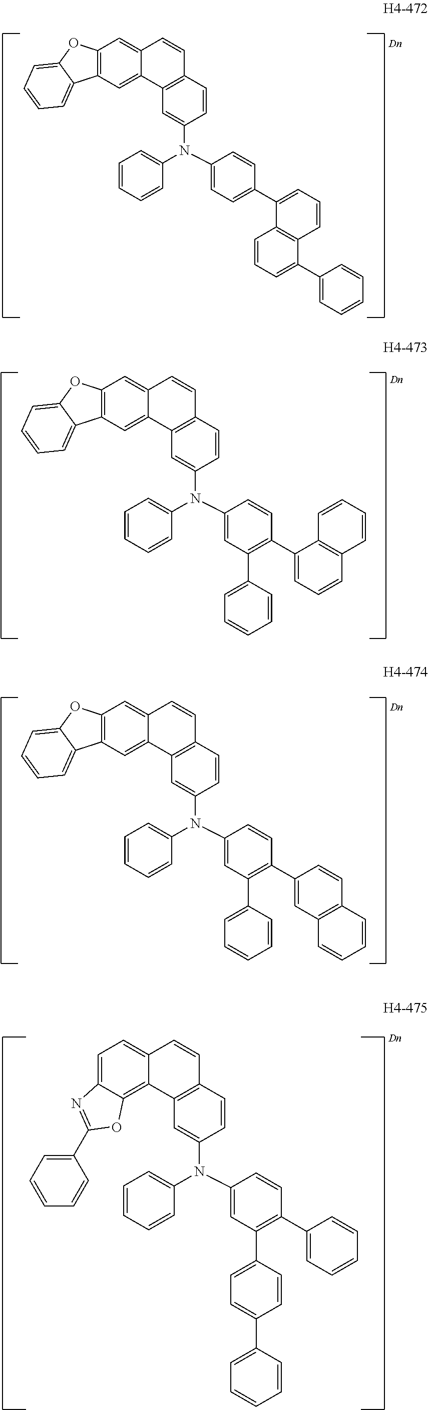 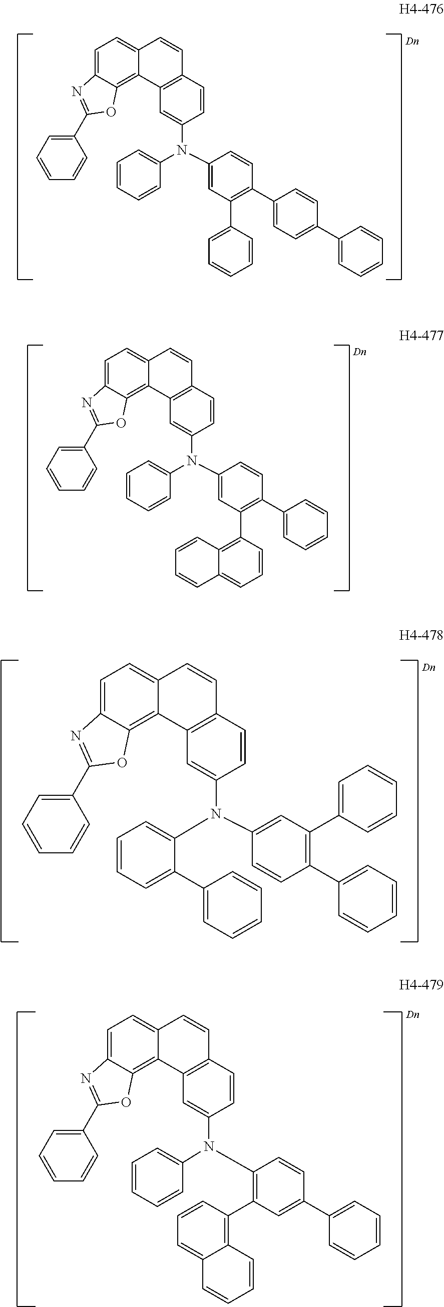 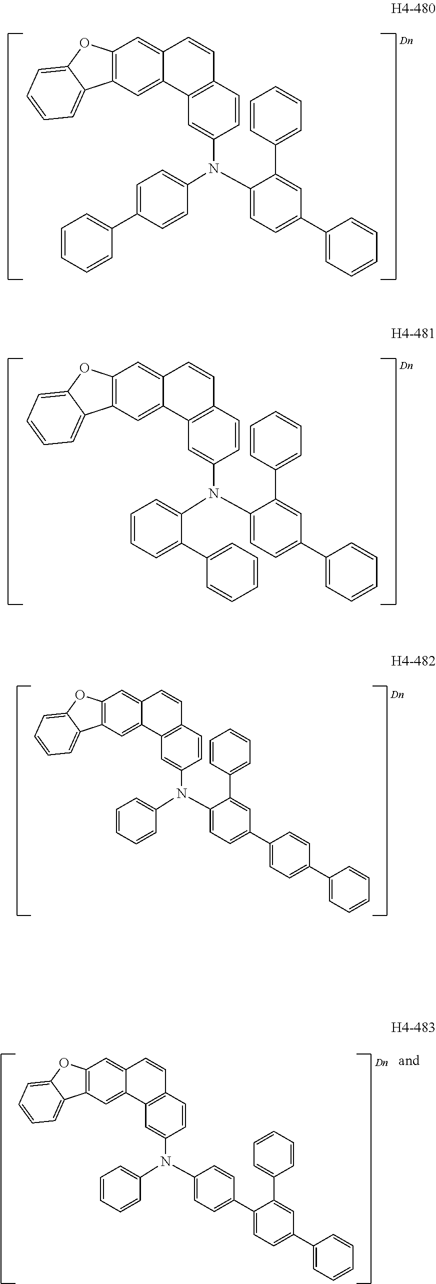 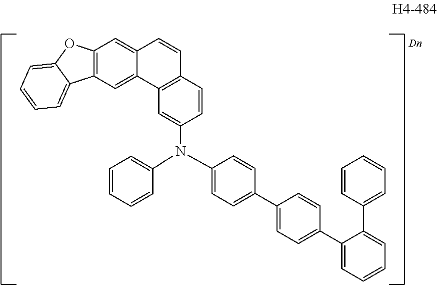
in the compounds above, Dₙ means that n number of hydrogens is replaced with deuterium, wherein n is an integer of 1 or more, and the upper limit of n is determined according to the number of hydrogens that may be substituted for each compound.

8. The organic electroluminescent device according to claim 3, wherein the compound represented by Formula 3 is selected from the following compounds: 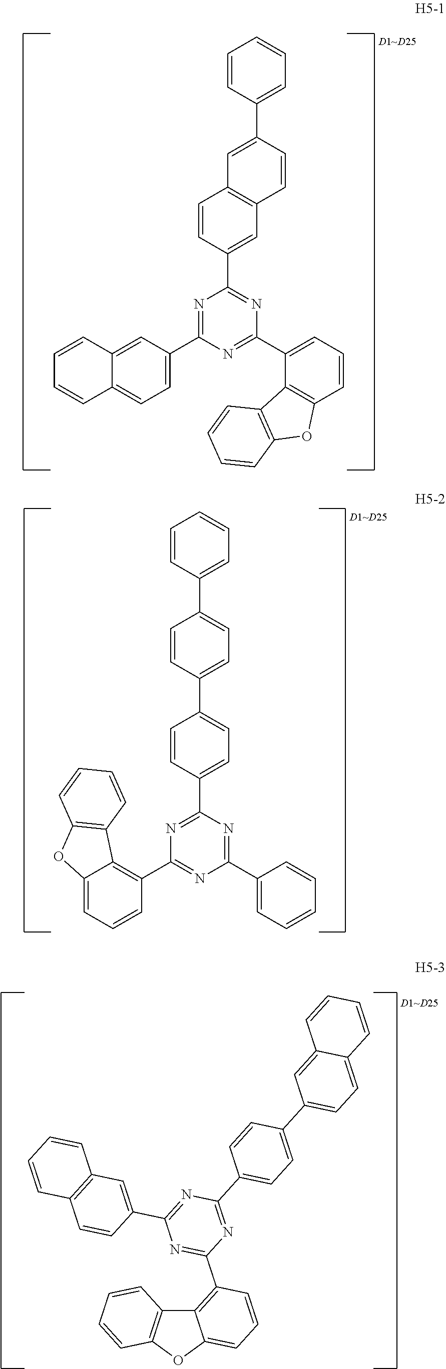 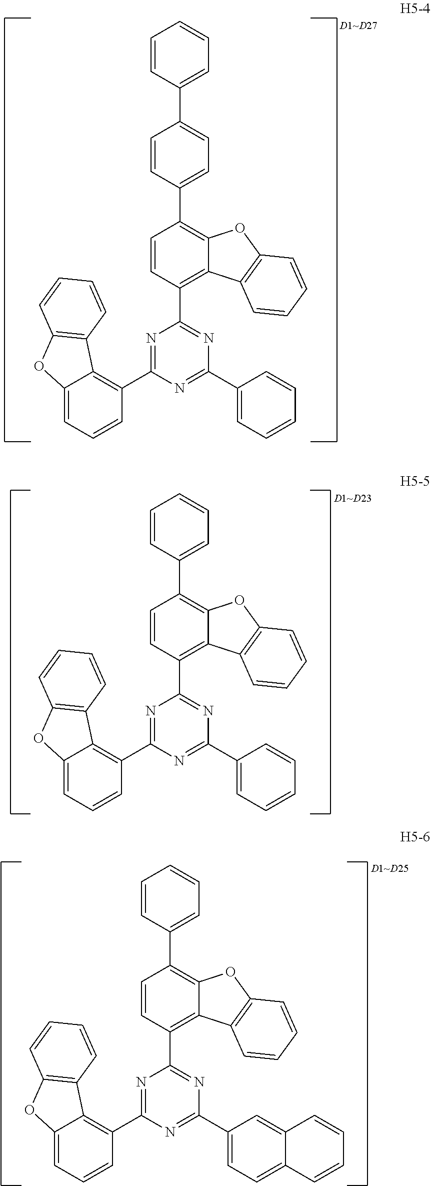 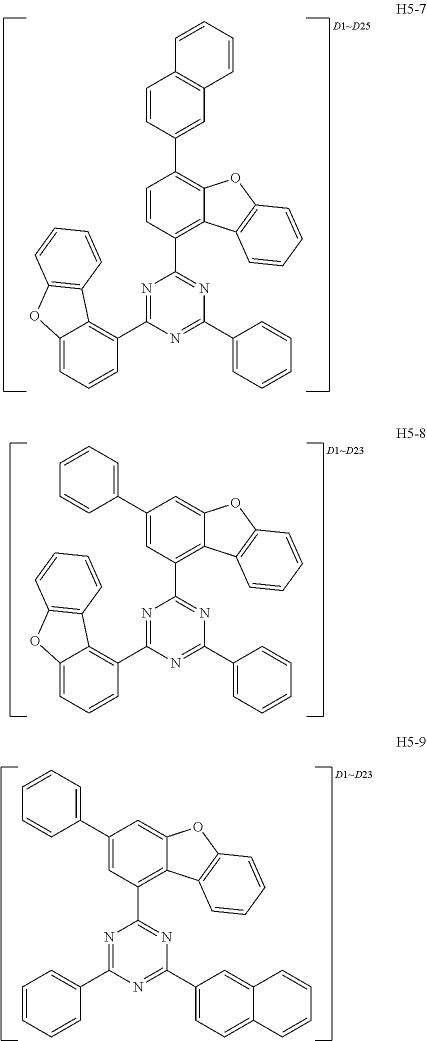 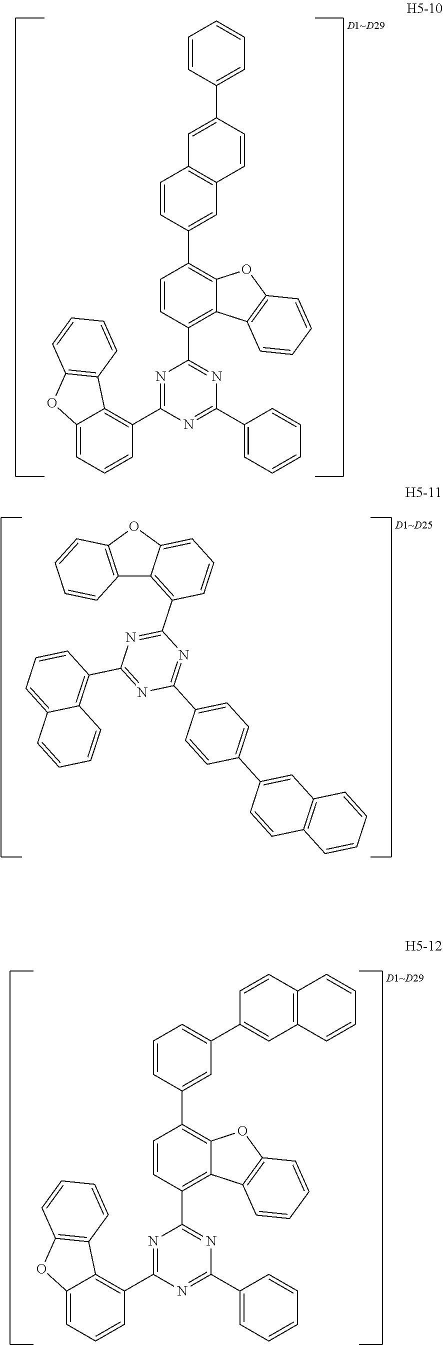 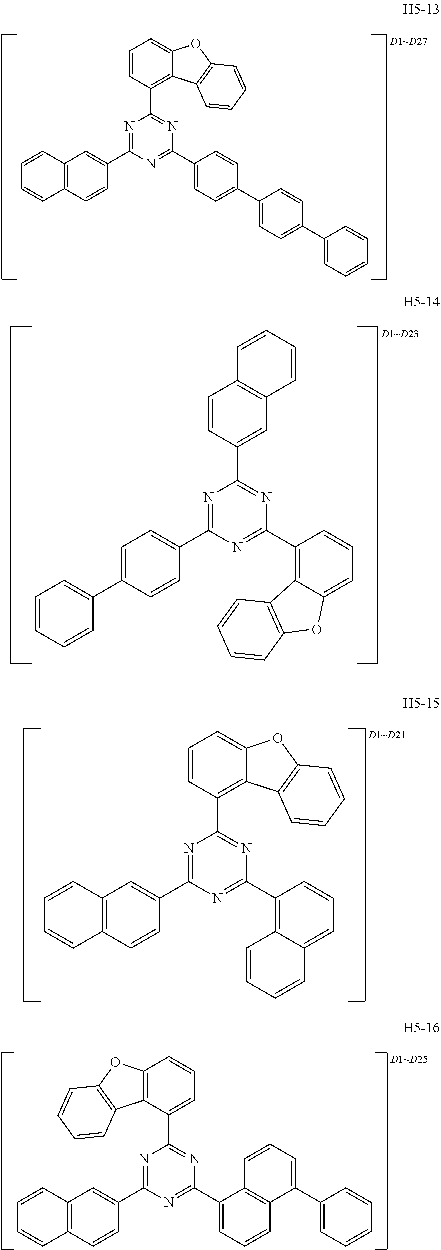 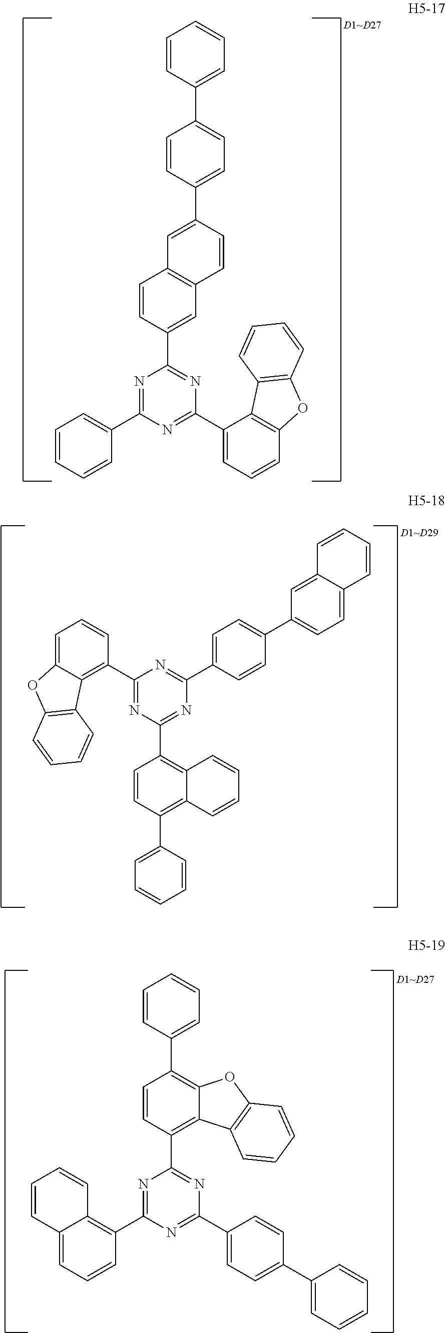 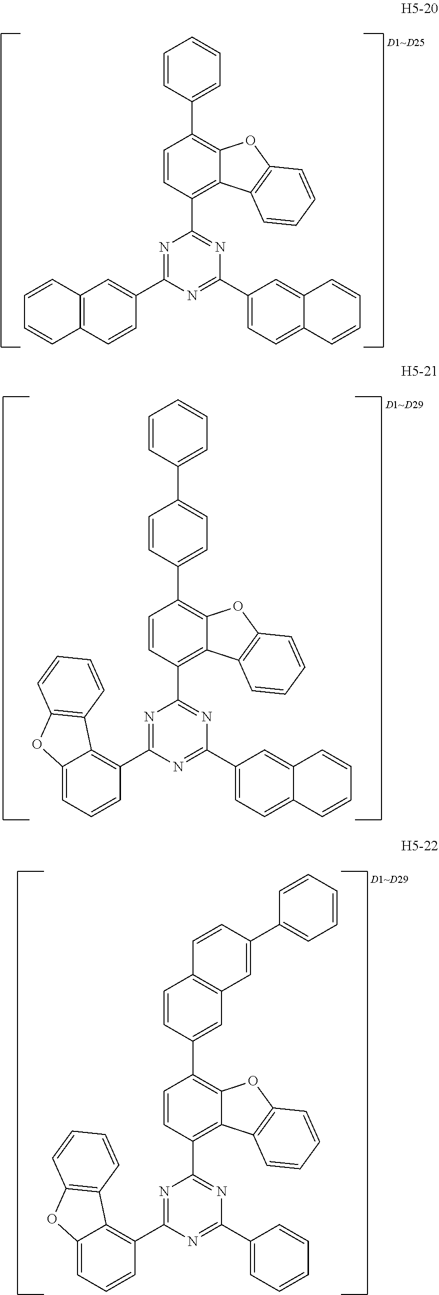 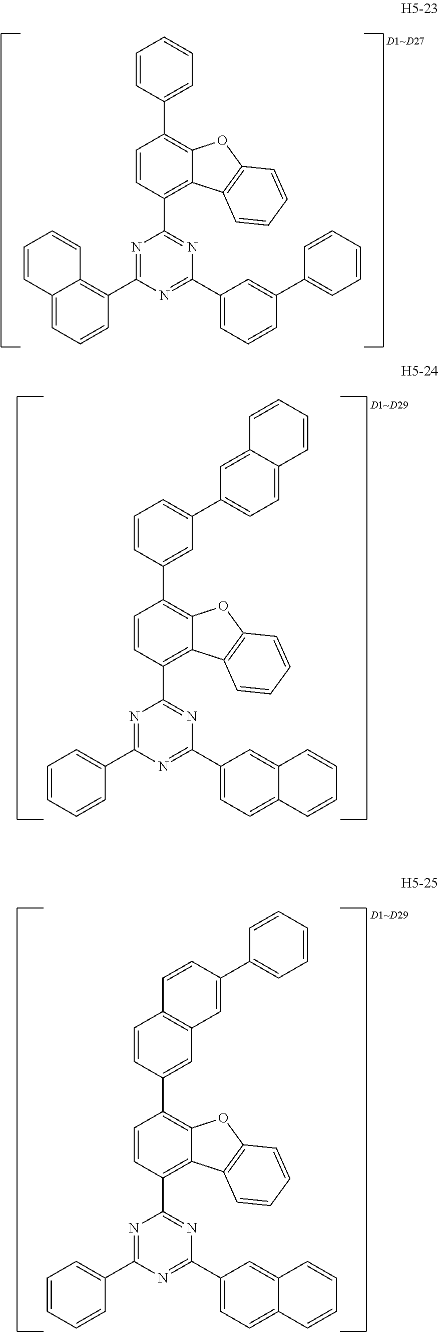 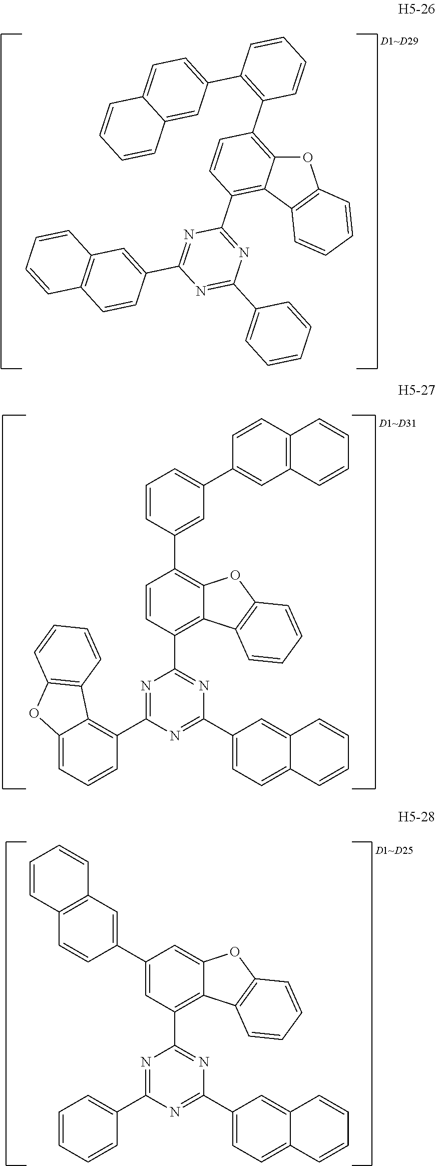 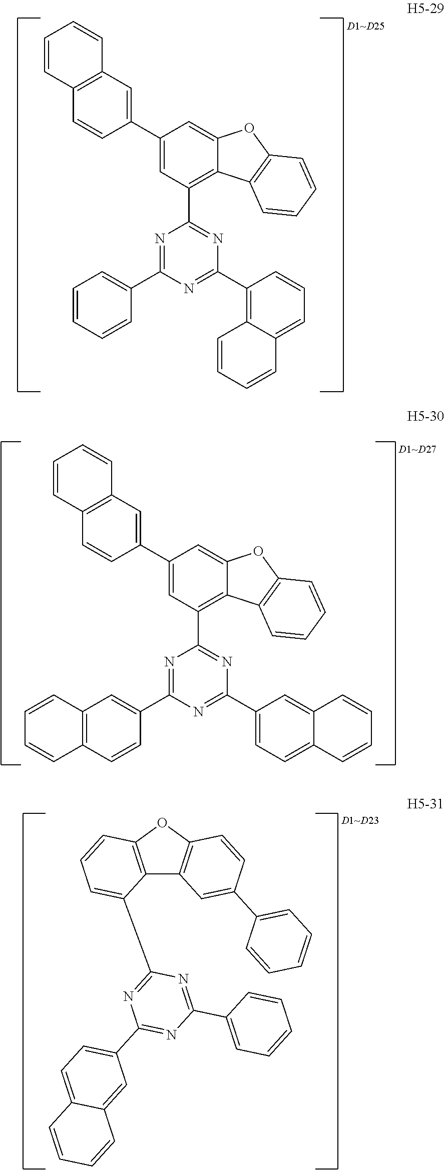 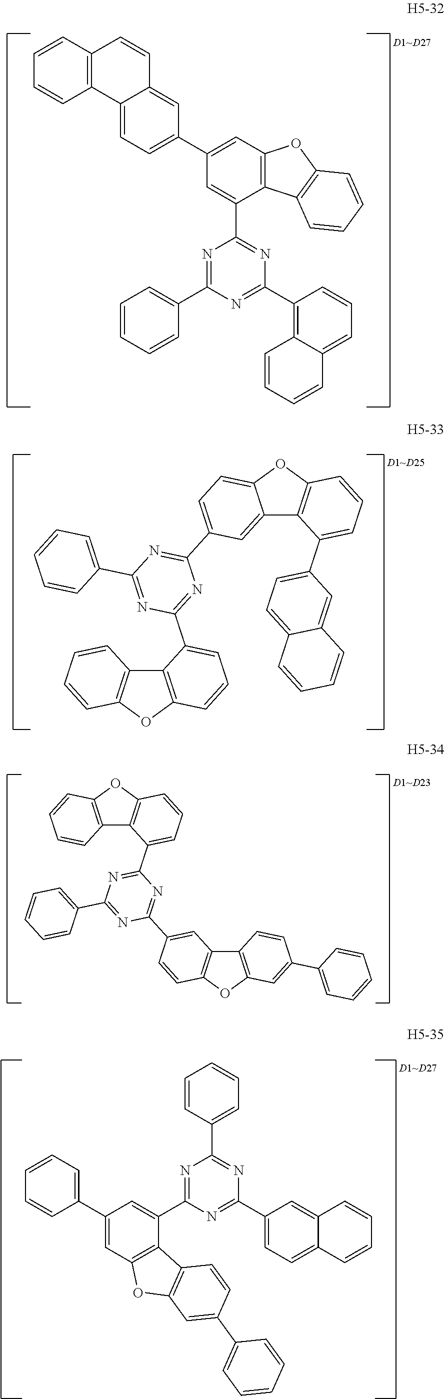 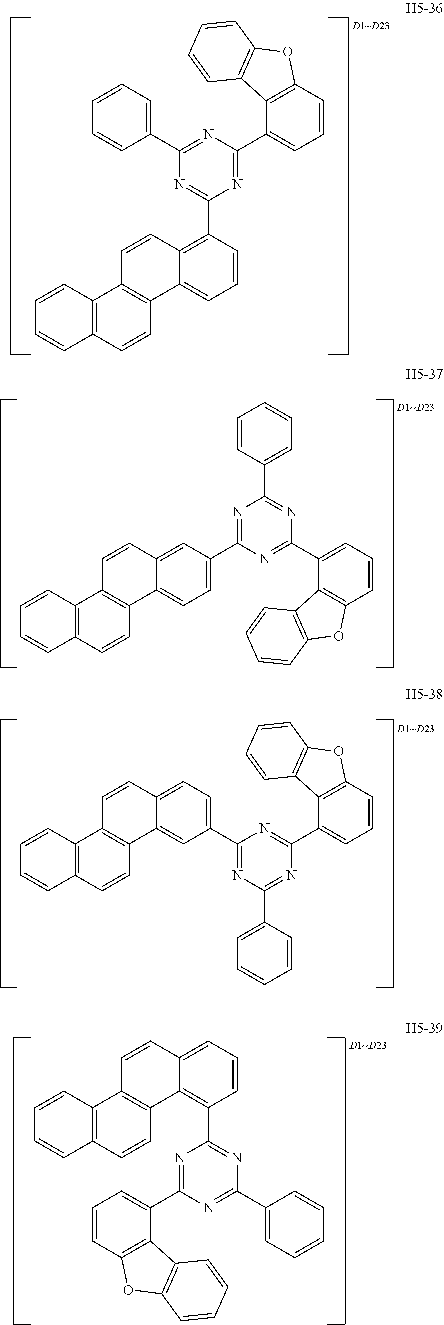 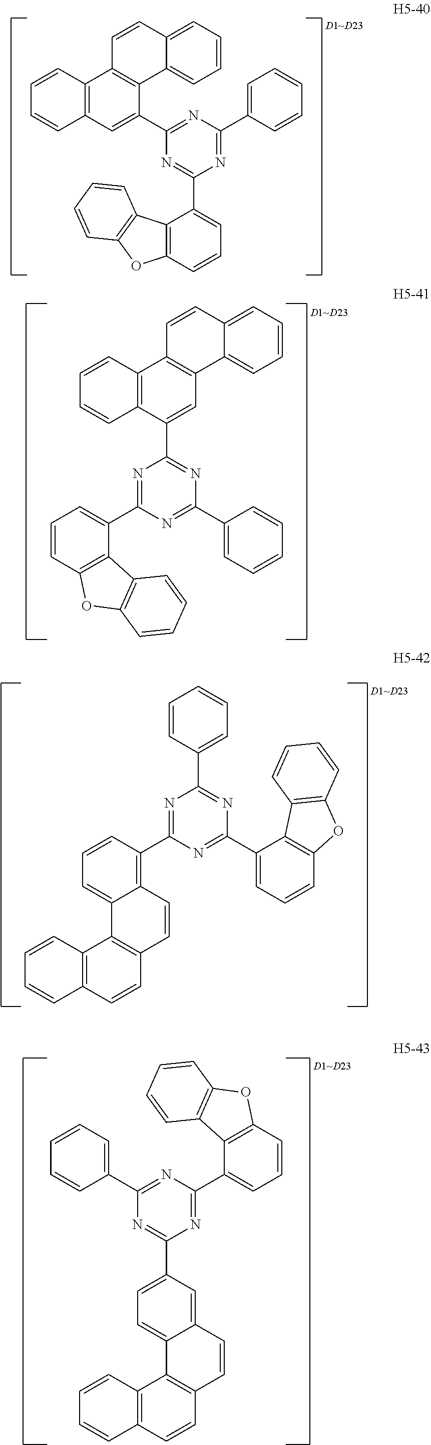 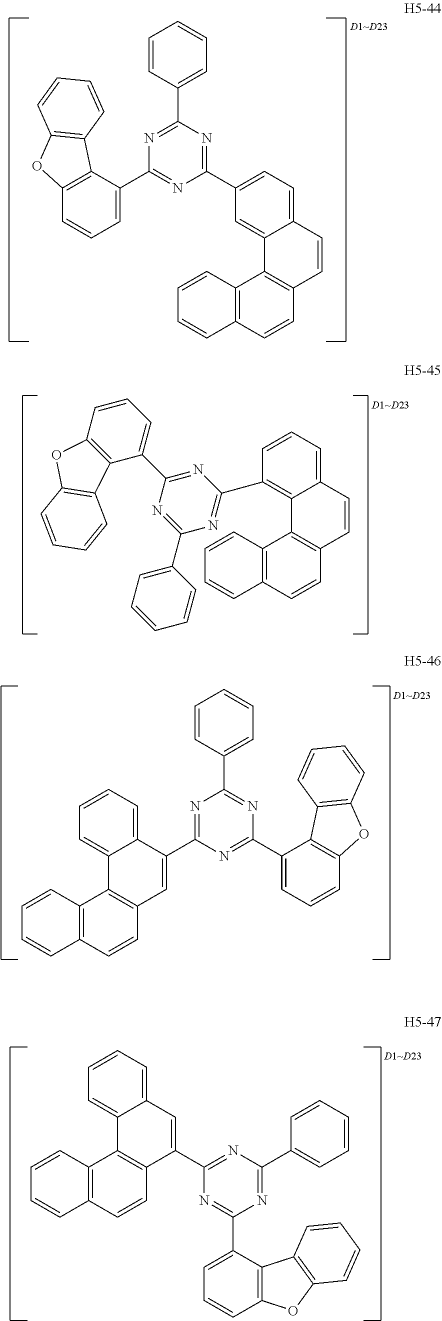 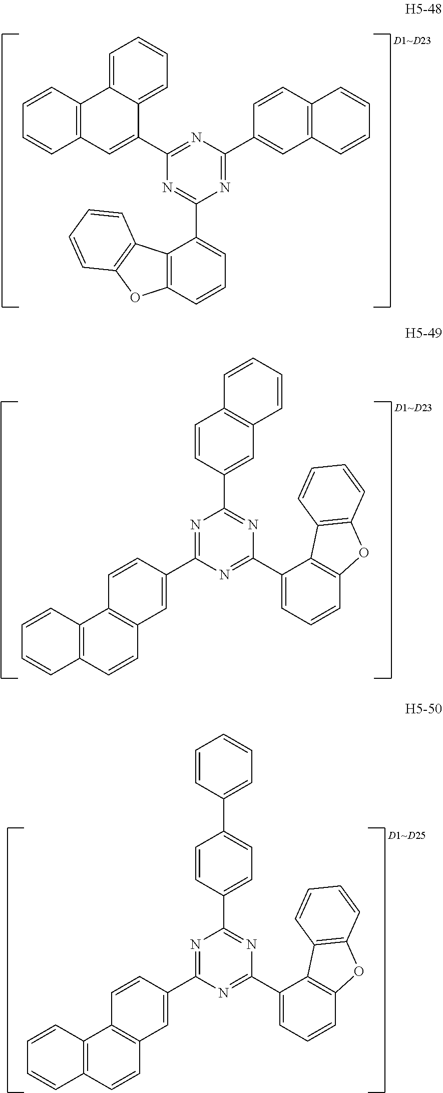 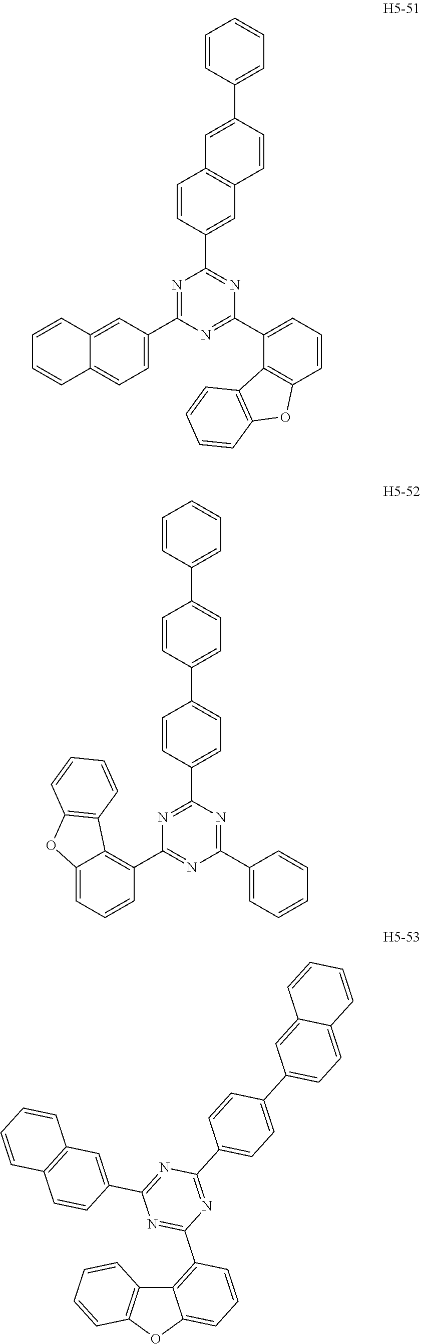 [SMILES: c1ccc(-c2ccc3cc(-c4nc(-c5ccc6ccccc6c5)nc(-c5cccc6oc7ccccc7c56)n4)ccc3c2)cc1 ; c1ccc(-c2ccc(-c3ccc(-c4nc(-c5ccccc5)nc(-c5cccc6oc7ccccc7c56)n4)cc3)cc2)cc1 ; c1ccc2cc(-c3ccc(-c4nc(-c5ccc6ccccc6c5)nc(-c5cccc6oc7ccccc7c56)n4)cc3)ccc2c1] 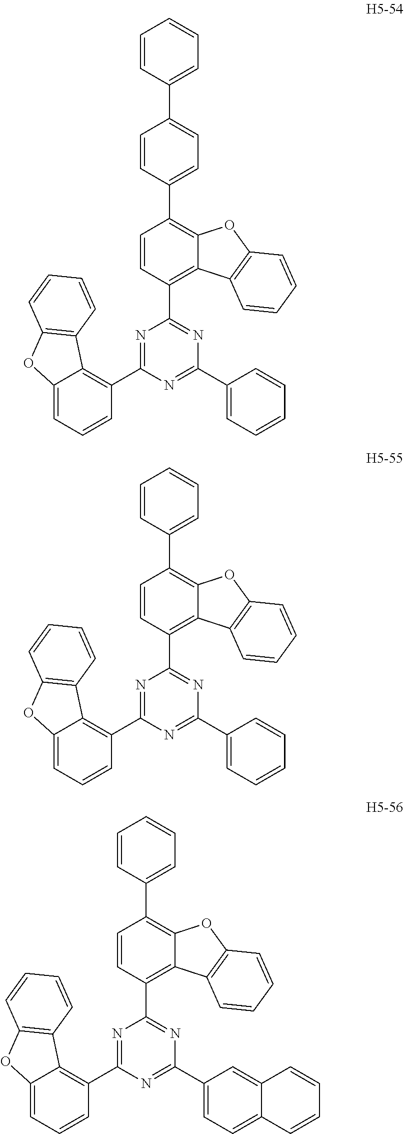 [SMILES: c1ccc(-c2ccc(-c3ccc(-c4nc(-c5ccccc5)nc(-c5cccc6oc7ccccc7c56)n4)c4c3oc3ccccc34)cc2)cc1 ; c1ccc(-c2nc(-c3cccc4oc5ccccc5c34)nc(-c3ccc(-c4ccccc4)c4oc5ccccc5c34)n2)cc1 ; c1ccc(-c2ccc(-c3nc(-c4ccc5ccccc5c4)nc(-c4cccc5oc6ccccc6c45)n3)c3c2oc2ccccc23)cc1] 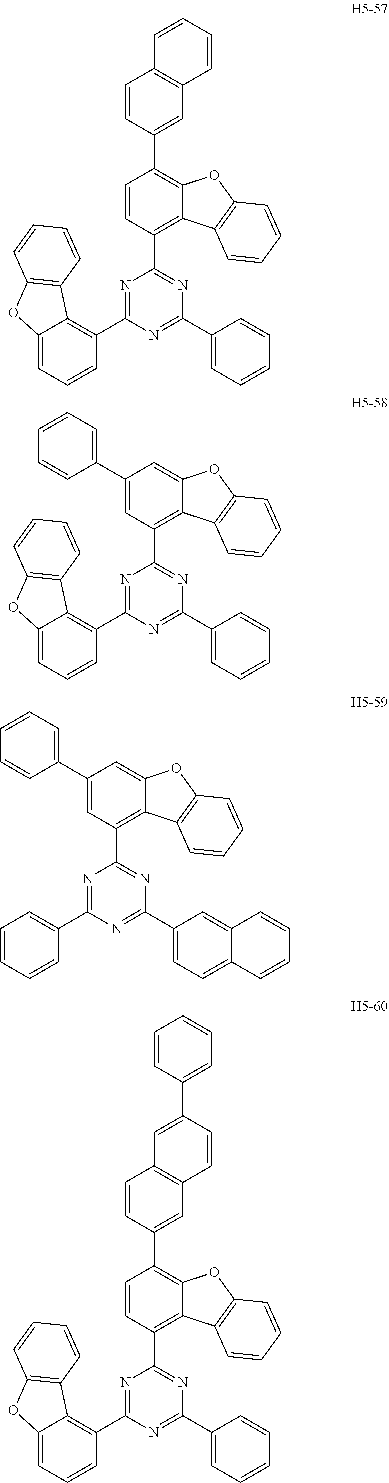 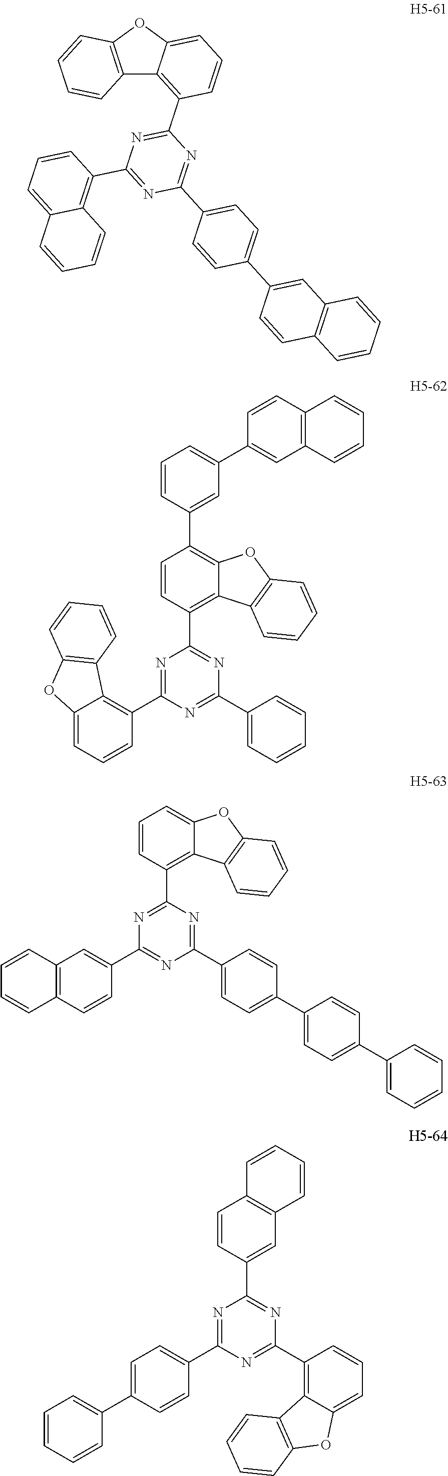 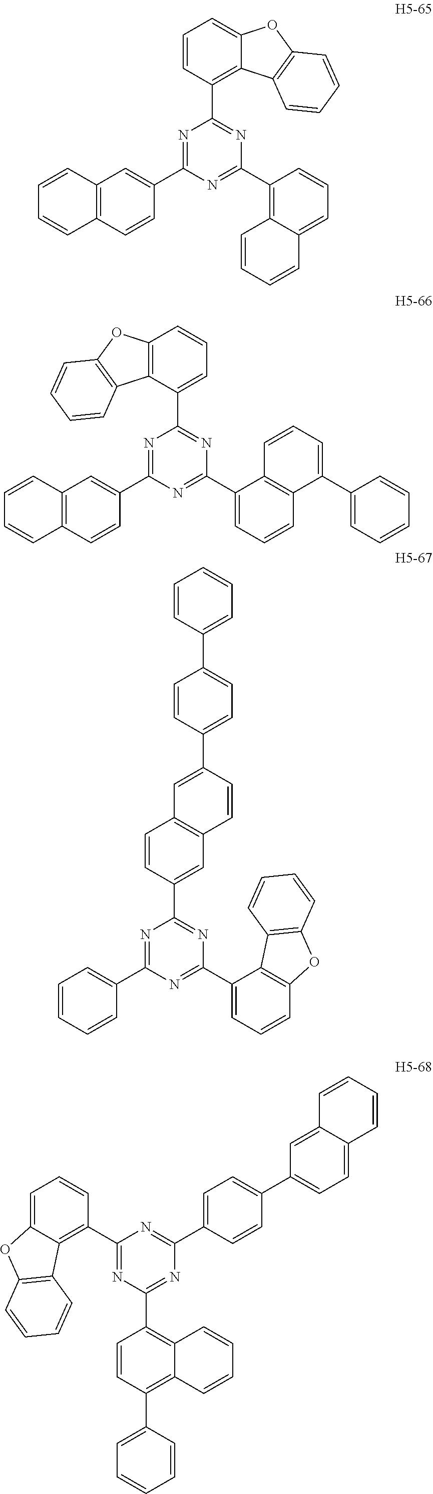 [SMILES: c1ccc2cc(-c3nc(-c4cccc5ccccc45)nc(-c4cccc5oc6ccccc6c45)n3)ccc2c1 ; c1ccc(-c2cccc3c(-c4nc(-c5ccc6ccccc6c5)nc(-c5cccc6oc7ccccc7c56)n4)cccc23)cc1 ; c1ccc(-c2ccc(-c3ccc4cc(-c5nc(-c6ccccc6)nc(-c6cccc7oc8ccccc8c67)n5)ccc4c3)cc2)cc1 ; c1ccc(-c2ccc(-c3nc(-c4ccc(-c5ccc6ccccc6c5)cc4)nc(-c4cccc5oc6ccccc6c45)n3)c3ccccc23)cc1] 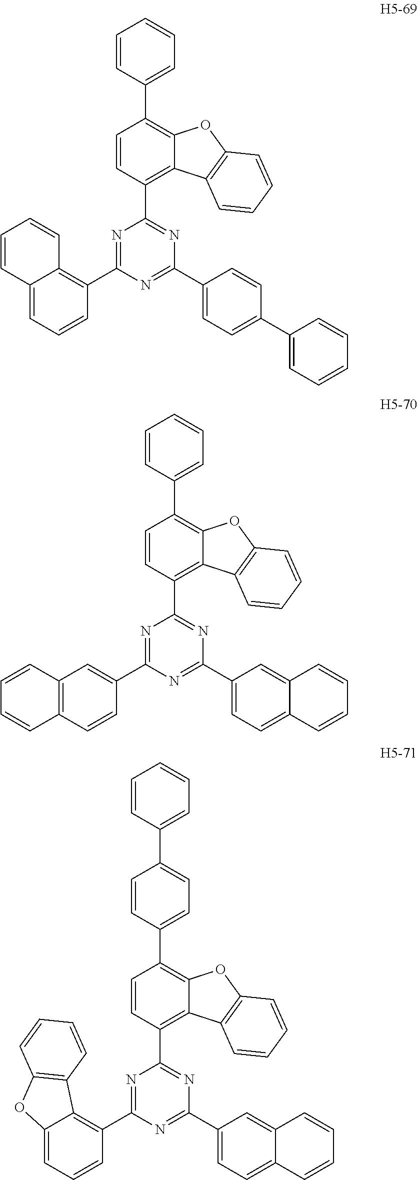 [SMILES: c1ccc(-c2ccc(-c3nc(-c4cccc5ccccc45)nc(-c4ccc(-c5ccccc5)c5oc6ccccc6c45)n3)cc2)cc1 ; c1ccc(-c2ccc(-c3nc(-c4ccc5ccccc5c4)nc(-c4ccc5ccccc5c4)n3)c3c2oc2ccccc23)cc1 ; c1ccc(-c2ccc(-c3ccc(-c4nc(-c5ccc6ccccc6c5)nc(-c5cccc6oc7ccccc7c56)n4)c4c3oc3ccccc34)cc2)cc1] 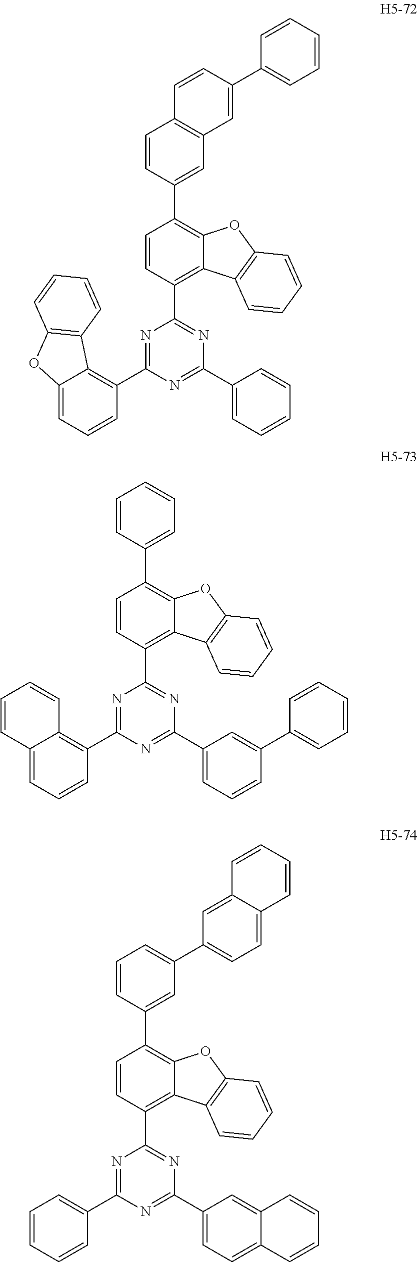 [SMILES: c1ccc(-c2ccc3ccc(-c4ccc(-c5nc(-c6ccccc6)nc(-c6cccc7oc8ccccc8c67)n5)c5c4oc4ccccc45)cc3c2)cc1 ; c1ccc(-c2cccc(-c3nc(-c4cccc5ccccc45)nc(-c4ccc(-c5ccccc5)c5oc6ccccc6c45)n3)c2)cc1 ; c1ccc(-c2nc(-c3ccc4ccccc4c3)nc(-c3ccc(-c4cccc(-c5ccc6ccccc6c5)c4)c4oc5ccccc5c34)n2)cc1] 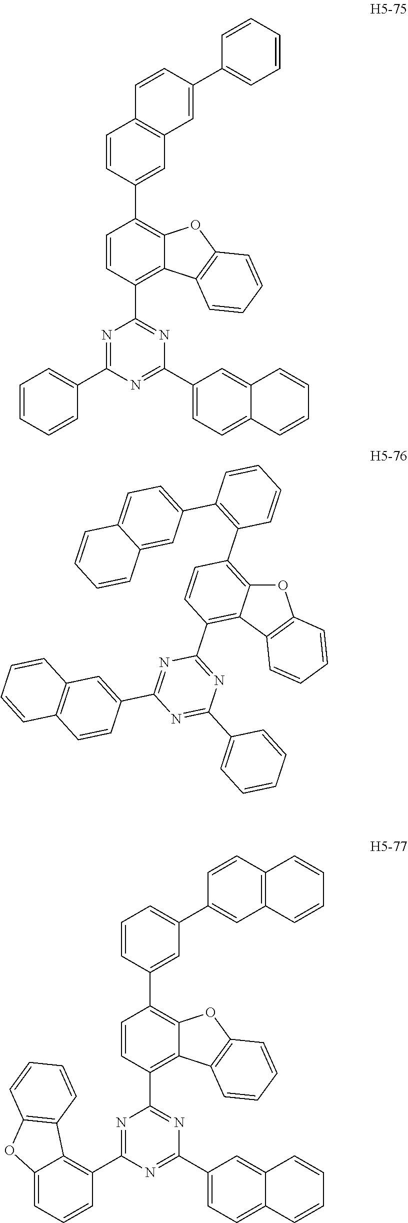 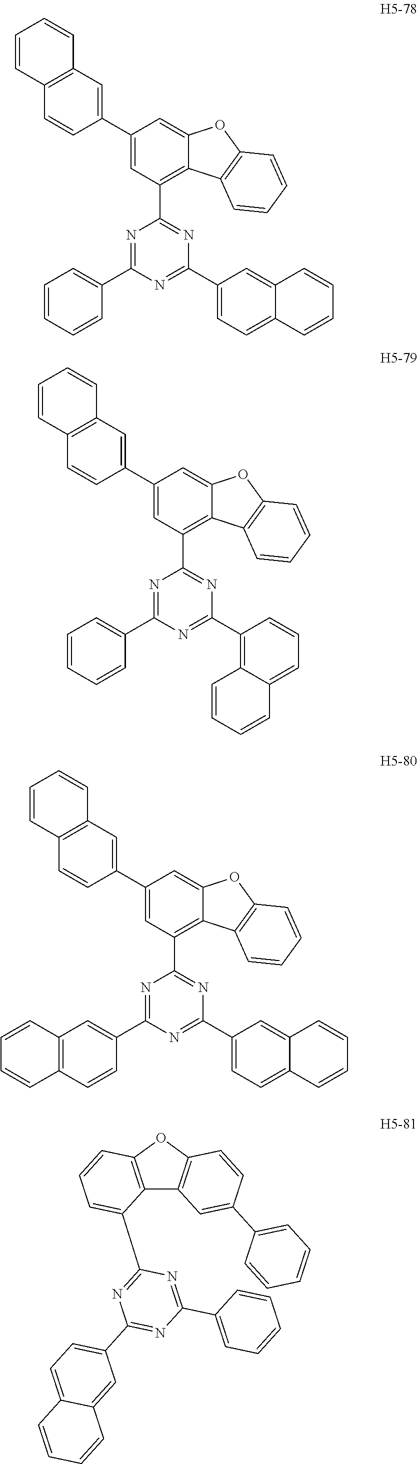 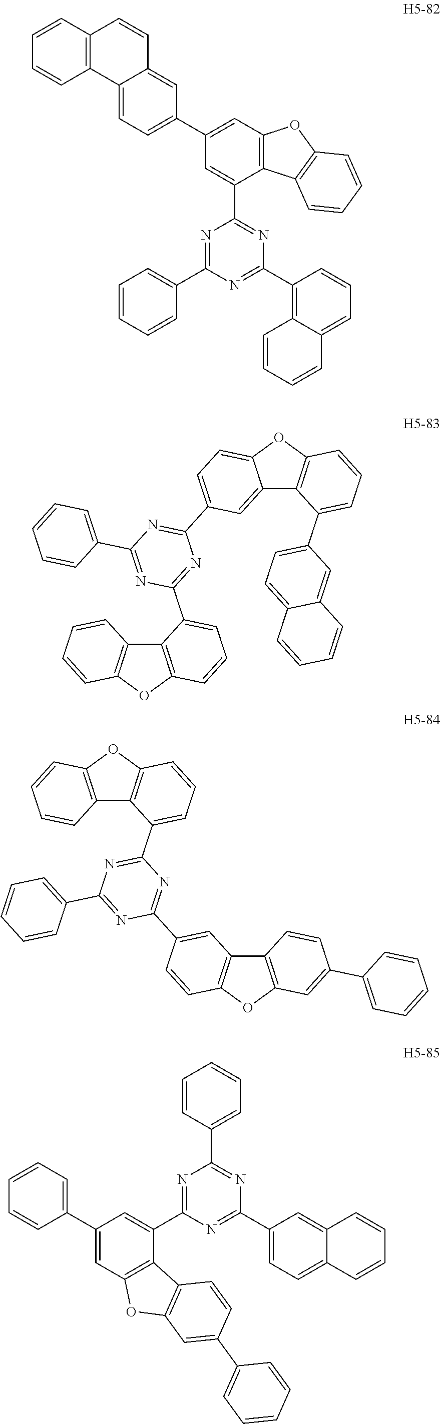 [SMILES: c1ccc(-c2nc(-c3cccc4ccccc34)nc(-c3cc(-c4ccc5c(ccc6ccccc65)c4)cc4oc5ccccc5c34)n2)cc1 ; c1ccc(-c2nc(-c3ccc4oc5cccc(-c6ccc7ccccc7c6)c5c4c3)nc(-c3cccc4oc5ccccc5c34)n2)cc1 ; c1ccc(-c2ccc3c(c2)oc2ccc(-c4nc(-c5ccccc5)nc(-c5cccc6oc7ccccc7c56)n4)cc23)cc1 ; c1ccc(-c2ccc3c(c2)oc2cc(-c4ccccc4)cc(-c4nc(-c5ccccc5)nc(-c5ccc6ccccc6c5)n4)c23)cc1] 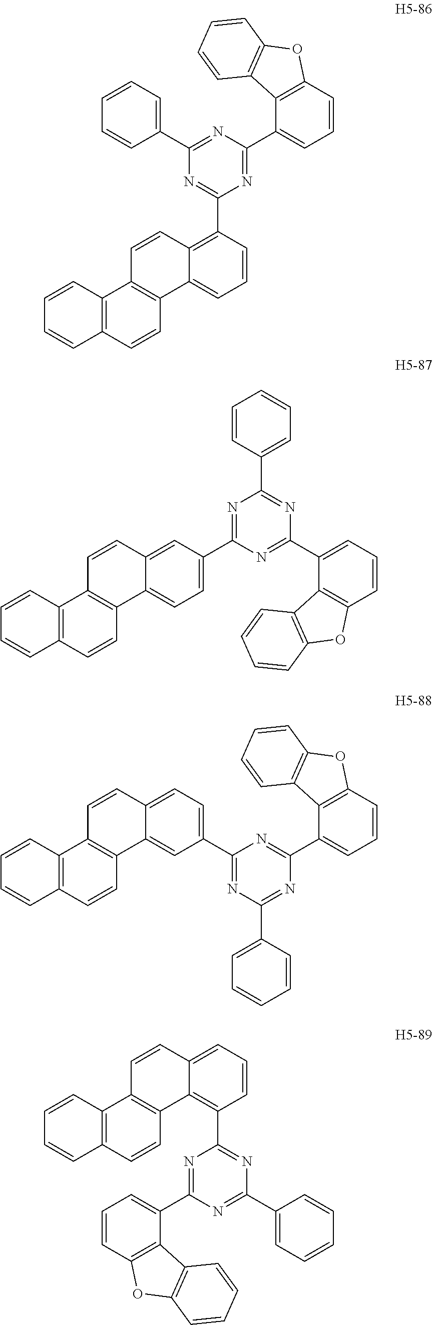 [SMILES: c1ccc(-c2nc(-c3cccc4c3ccc3c5ccccc5ccc43)nc(-c3cccc4oc5ccccc5c34)n2)cc1 ; c1ccc(-c2nc(-c3ccc4c(ccc5c6ccccc6ccc45)c3)nc(-c3cccc4oc5ccccc5c34)n2)cc1 ; c1ccc(-c2nc(-c3ccc4ccc5c6ccccc6ccc5c4c3)nc(-c3cccc4oc5ccccc5c34)n2)cc1 ; c1ccc(-c2nc(-c3cccc4ccc5c6ccccc6ccc5c34)nc(-c3cccc4oc5ccccc5c34)n2)cc1] 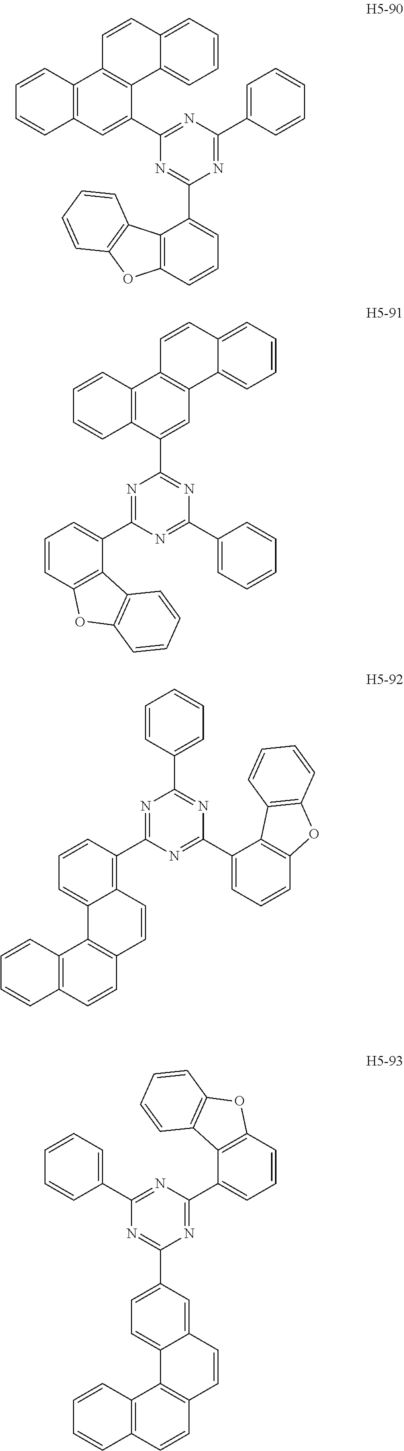 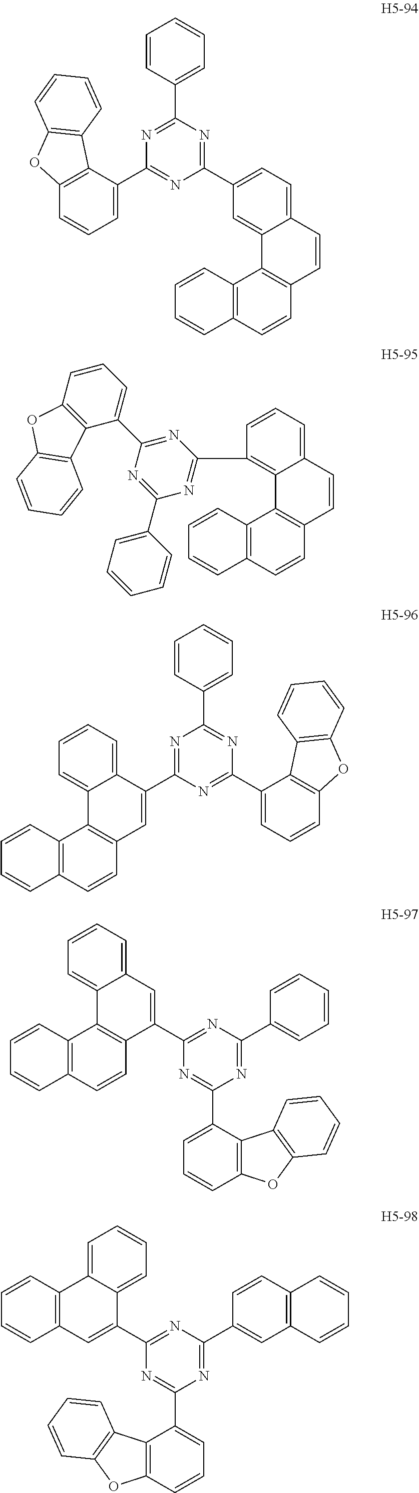 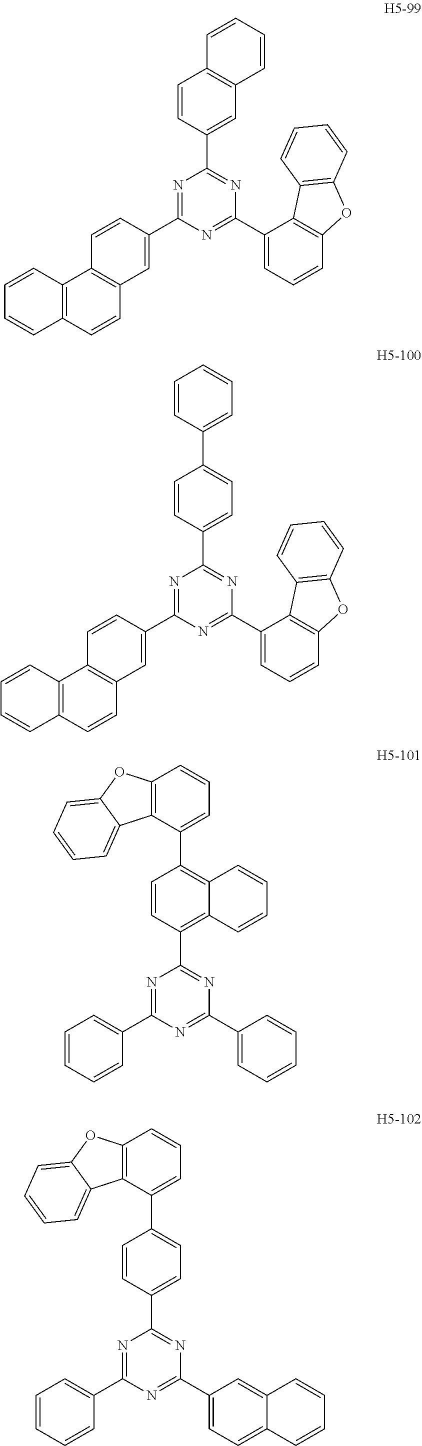 [SMILES: c1ccc2cc(-c3nc(-c4ccc5c(ccc6ccccc65)c4)nc(-c4cccc5oc6ccccc6c45)n3)ccc2c1 ; c1ccc(-c2ccc(-c3nc(-c4ccc5c(ccc6ccccc65)c4)nc(-c4cccc5oc6ccccc6c45)n3)cc2)cc1 ; c1ccc(-c2nc(-c3ccccc3)nc(-c3ccc(-c4cccc5oc6ccccc6c45)c4ccccc34)n2)cc1 ; c1ccc(-c2nc(-c3ccc(-c4cccc5oc6ccccc6c45)cc3)nc(-c3ccc4ccccc4c3)n2)cc1] 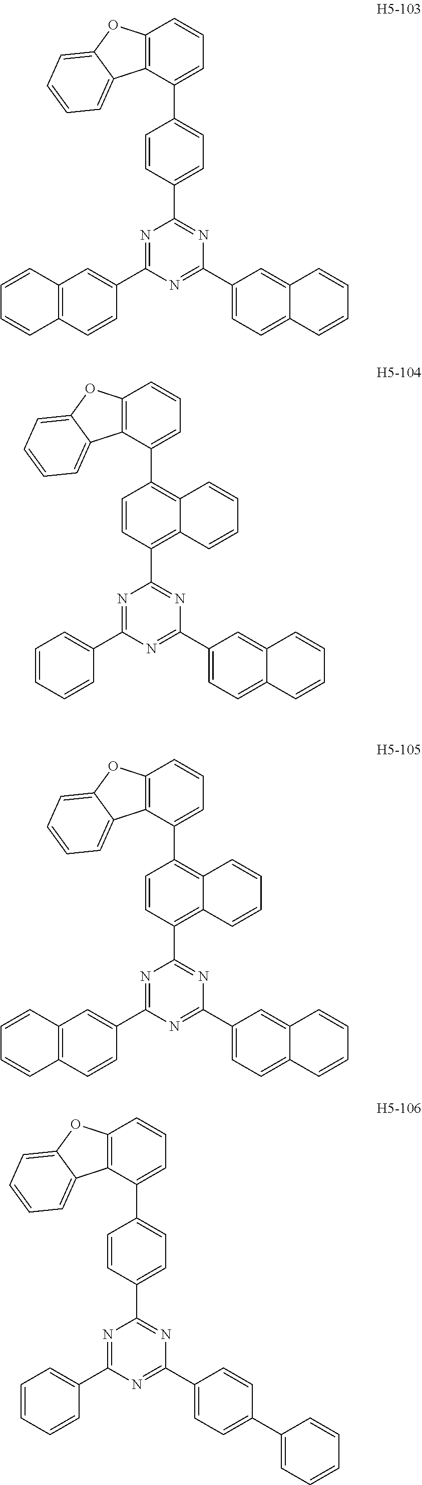 [SMILES: c1ccc2cc(-c3nc(-c4ccc(-c5cccc6oc7ccccc7c56)cc4)nc(-c4ccc5ccccc5c4)n3)ccc2c1 ; c1ccc(-c2nc(-c3ccc4ccccc4c3)nc(-c3ccc(-c4cccc5oc6ccccc6c45)c4ccccc34)n2)cc1 ; c1ccc2cc(-c3nc(-c4ccc5ccccc5c4)nc(-c4ccc(-c5cccc6oc7ccccc7c56)c5ccccc45)n3)ccc2c1 ; c1ccc(-c2ccc(-c3nc(-c4ccccc4)nc(-c4ccc(-c5cccc6oc7ccccc7c56)cc4)n3)cc2)cc1] 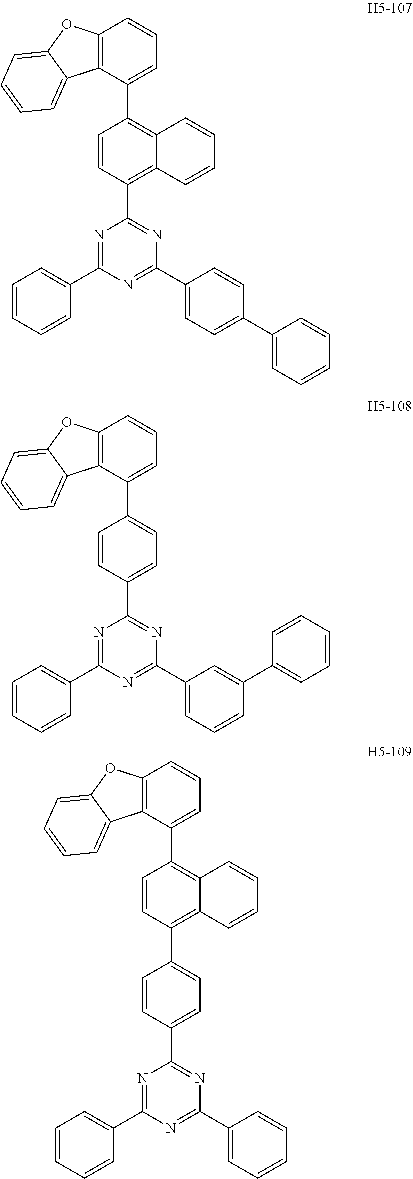 [SMILES: c1ccc(-c2ccc(-c3nc(-c4ccccc4)nc(-c4ccc(-c5cccc6oc7ccccc7c56)c5ccccc45)n3)cc2)cc1 ; c1ccc(-c2cccc(-c3nc(-c4ccccc4)nc(-c4ccc(-c5cccc6oc7ccccc7c56)cc4)n3)c2)cc1 ; c1ccc(-c2nc(-c3ccccc3)nc(-c3ccc(-c4ccc(-c5cccc6oc7ccccc7c56)c5ccccc45)cc3)n2)cc1] 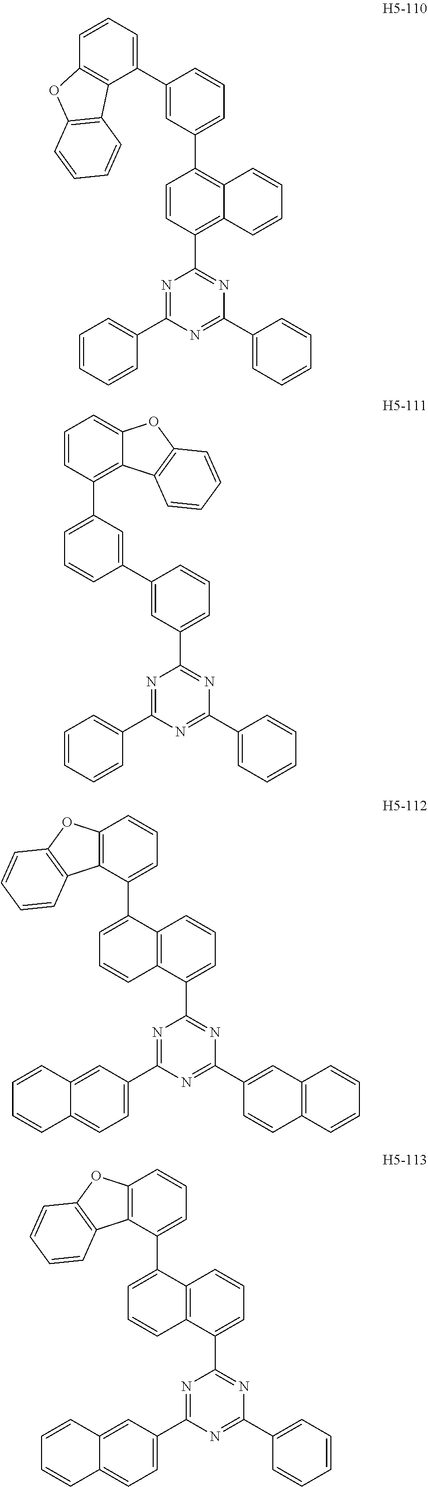 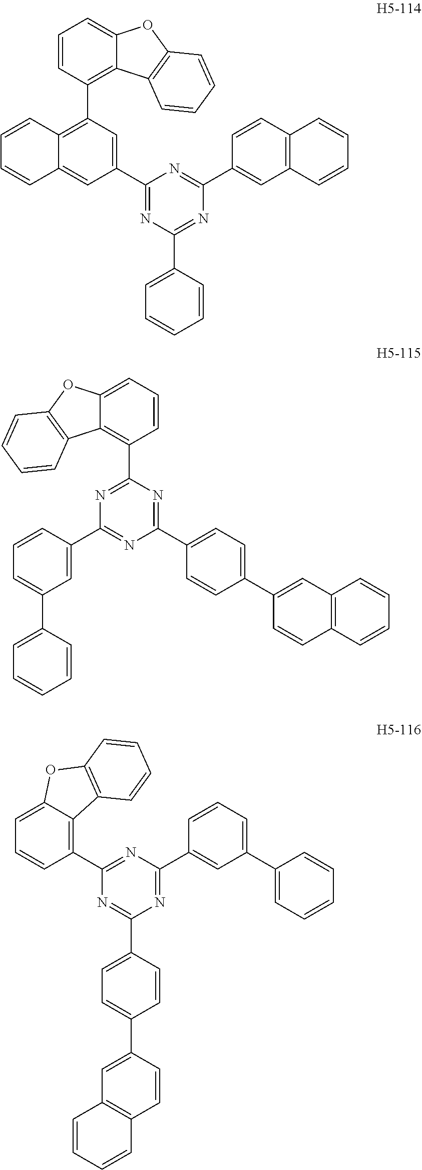 [SMILES: c1ccc(-c2nc(-c3ccc4ccccc4c3)nc(-c3cc(-c4cccc5oc6ccccc6c45)c4ccccc4c3)n2)cc1 ; c1ccc(-c2cccc(-c3nc(-c4ccc(-c5ccc6ccccc6c5)cc4)nc(-c4cccc5oc6ccccc6c45)n3)c2)cc1 ; c1ccc(-c2cccc(-c3nc(-c4ccc(-c5ccc6ccccc6c5)cc4)nc(-c4cccc5oc6ccccc6c45)n3)c2)cc1] 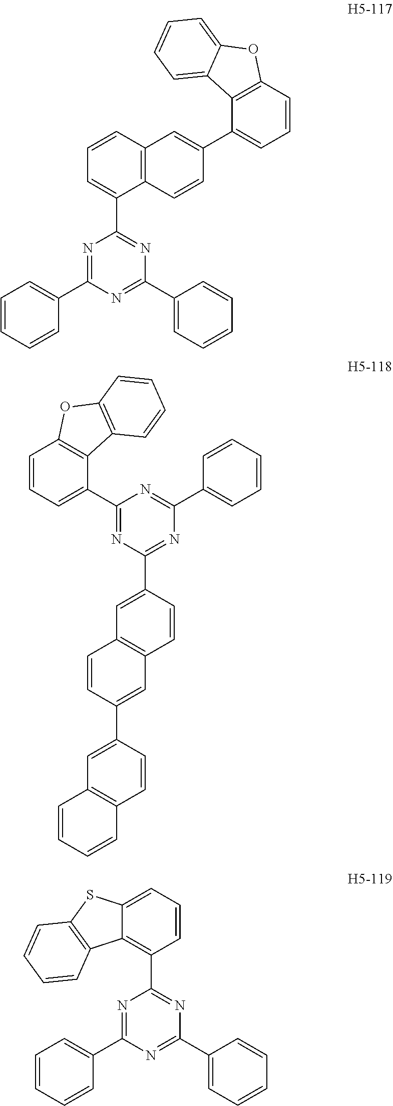 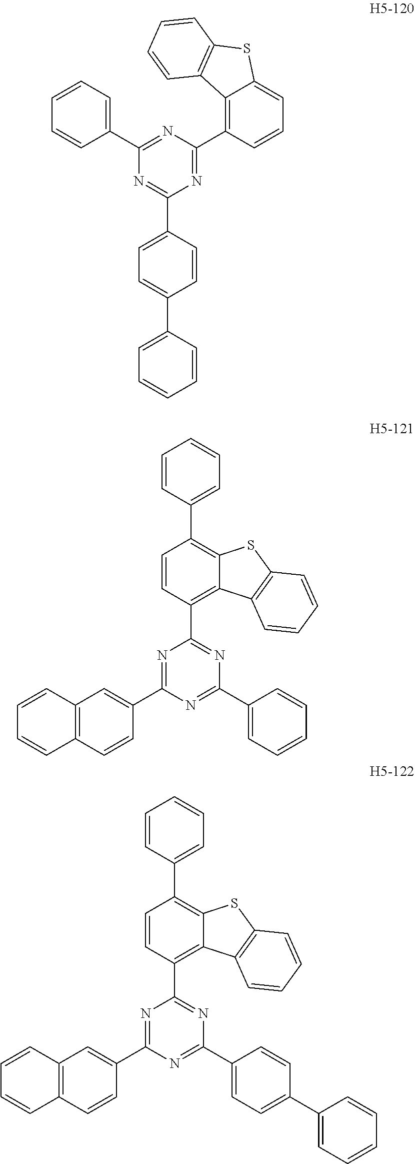 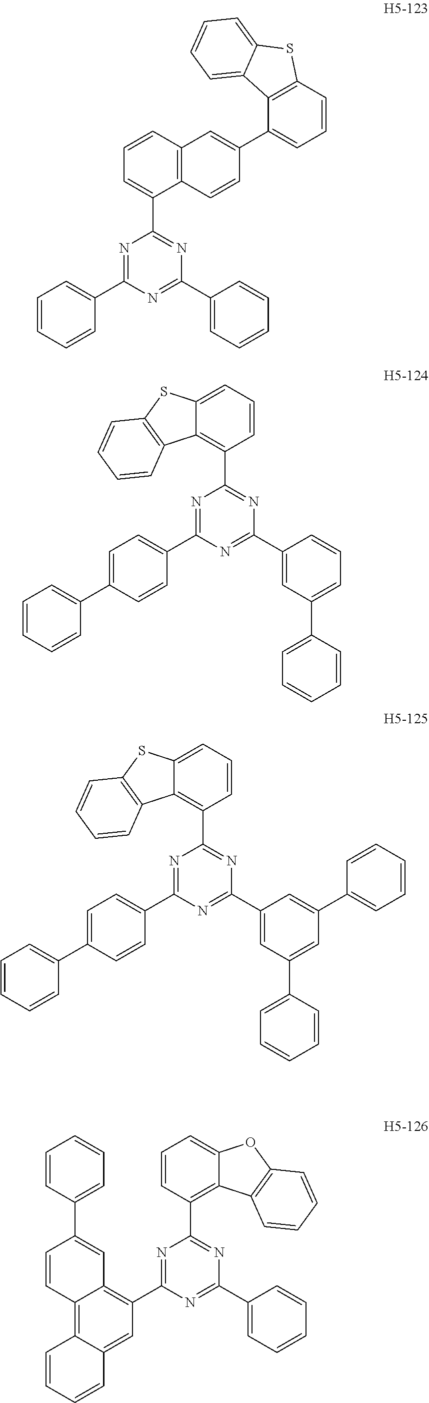 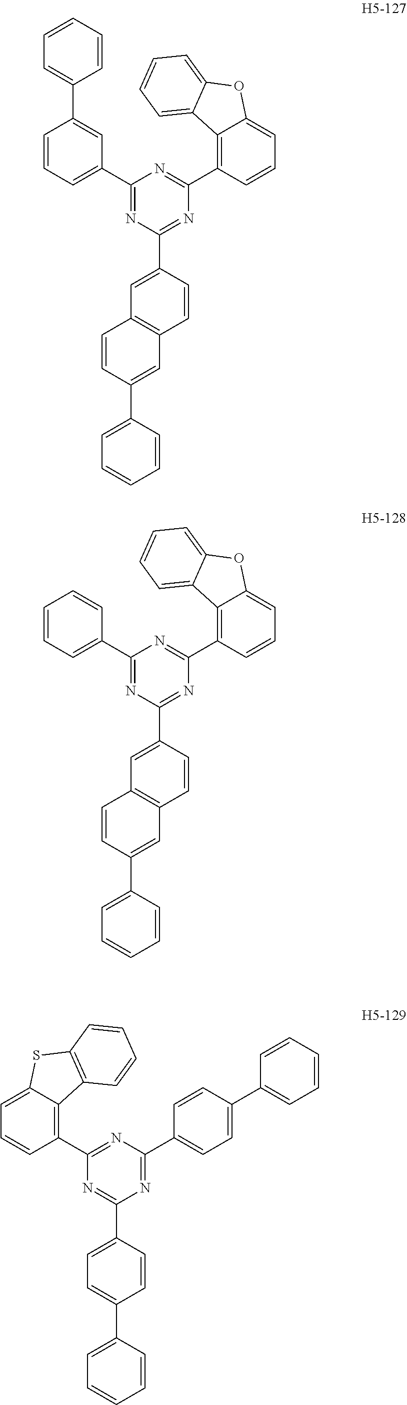 [SMILES: c1ccc(-c2cccc(-c3nc(-c4ccc5cc(-c6ccccc6)ccc5c4)nc(-c4cccc5oc6ccccc6c45)n3)c2)cc1 ; c1ccc(-c2ccc3cc(-c4nc(-c5ccccc5)nc(-c5cccc6oc7ccccc7c56)n4)ccc3c2)cc1 ; c1ccc(-c2ccc(-c3nc(-c4ccc(-c5ccccc5)cc4)nc(-c4cccc5sc6ccccc6c45)n3)cc2)cc1] 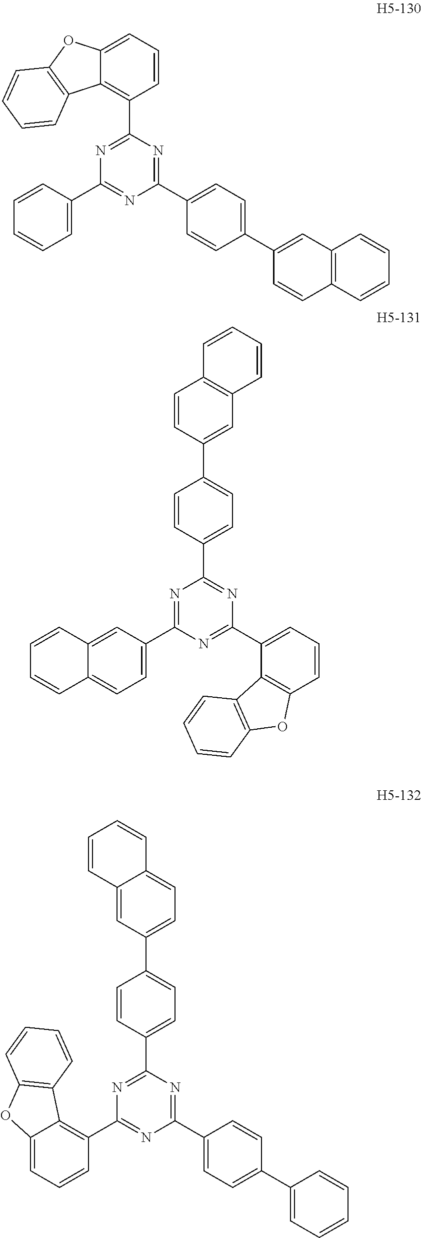 [SMILES: c1ccc(-c2nc(-c3ccc(-c4ccc5ccccc5c4)cc3)nc(-c3cccc4oc5ccccc5c34)n2)cc1 ; c1ccc2cc(-c3ccc(-c4nc(-c5ccc6ccccc6c5)nc(-c5cccc6oc7ccccc7c56)n4)cc3)ccc2c1 ; c1ccc(-c2ccc(-c3nc(-c4ccc(-c5ccc6ccccc6c5)cc4)nc(-c4cccc5oc6ccccc6c45)n3)cc2)cc1] 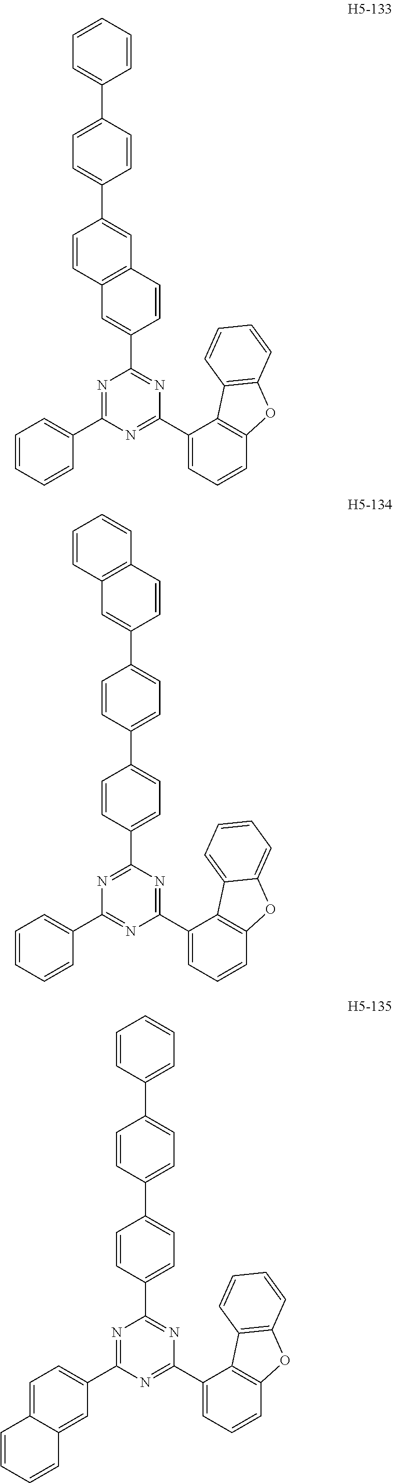 [SMILES: c1ccc(-c2ccc(-c3ccc4cc(-c5nc(-c6ccccc6)nc(-c6cccc7oc8ccccc8c67)n5)ccc4c3)cc2)cc1 ; c1ccc(-c2nc(-c3ccc(-c4ccc(-c5ccc6ccccc6c5)cc4)cc3)nc(-c3cccc4oc5ccccc5c34)n2)cc1 ; c1ccc(-c2ccc(-c3ccc(-c4nc(-c5ccc6ccccc6c5)nc(-c5cccc6oc7ccccc7c56)n4)cc3)cc2)cc1] 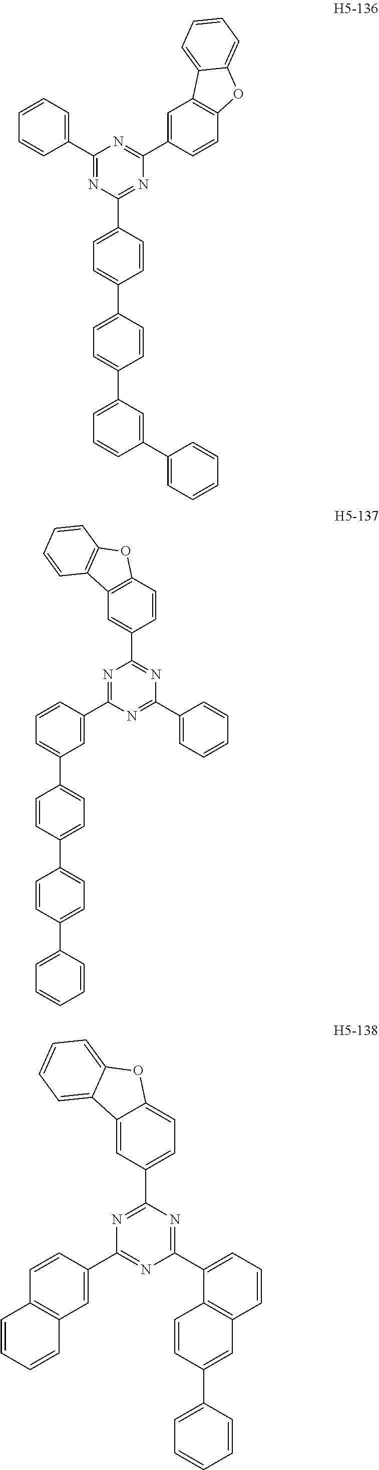 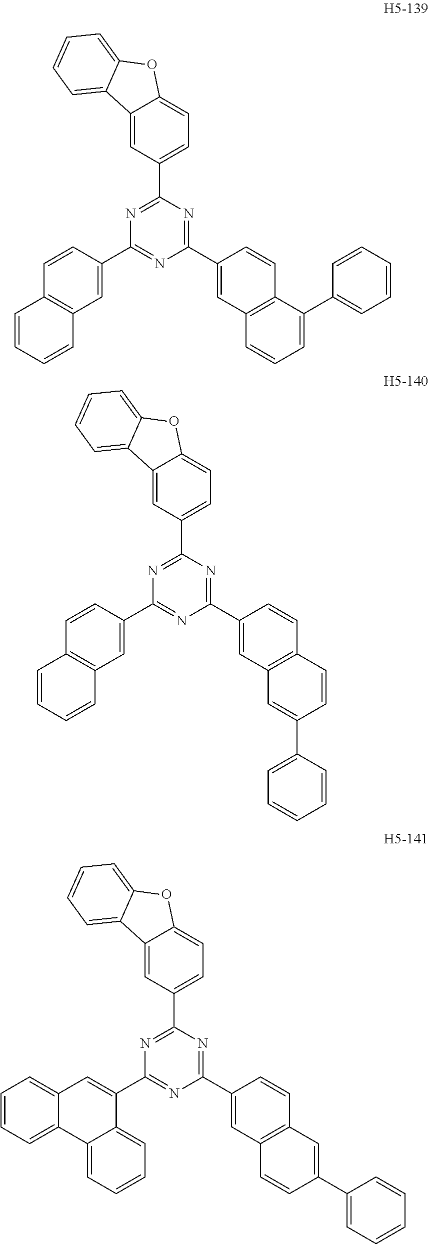 [SMILES: c1ccc(-c2cccc3cc(-c4nc(-c5ccc6ccccc6c5)nc(-c5ccc6oc7ccccc7c6c5)n4)ccc23)cc1 ; c1ccc(-c2ccc3ccc(-c4nc(-c5ccc6ccccc6c5)nc(-c5ccc6oc7ccccc7c6c5)n4)cc3c2)cc1 ; c1ccc(-c2ccc3cc(-c4nc(-c5ccc6oc7ccccc7c6c5)nc(-c5cc6ccccc6c6ccccc56)n4)ccc3c2)cc1] 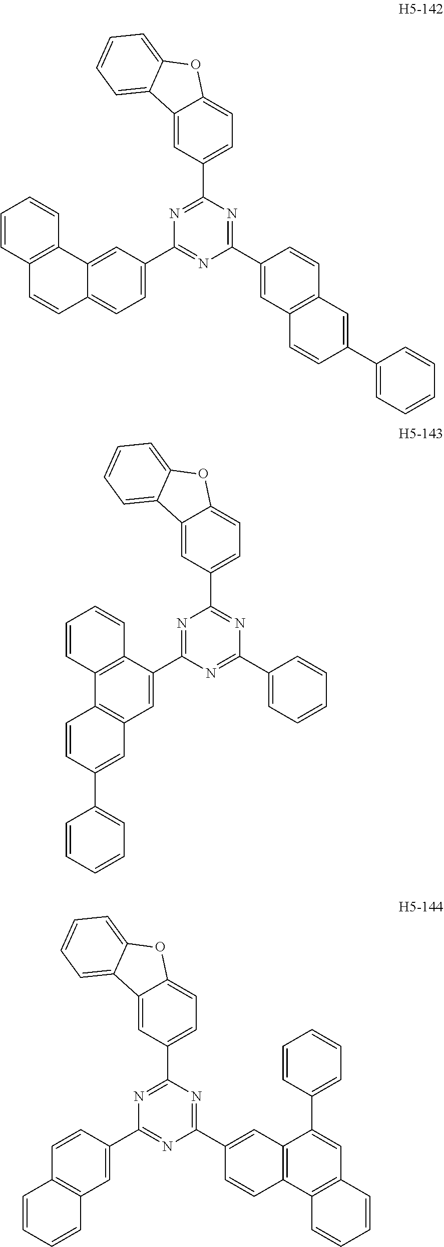 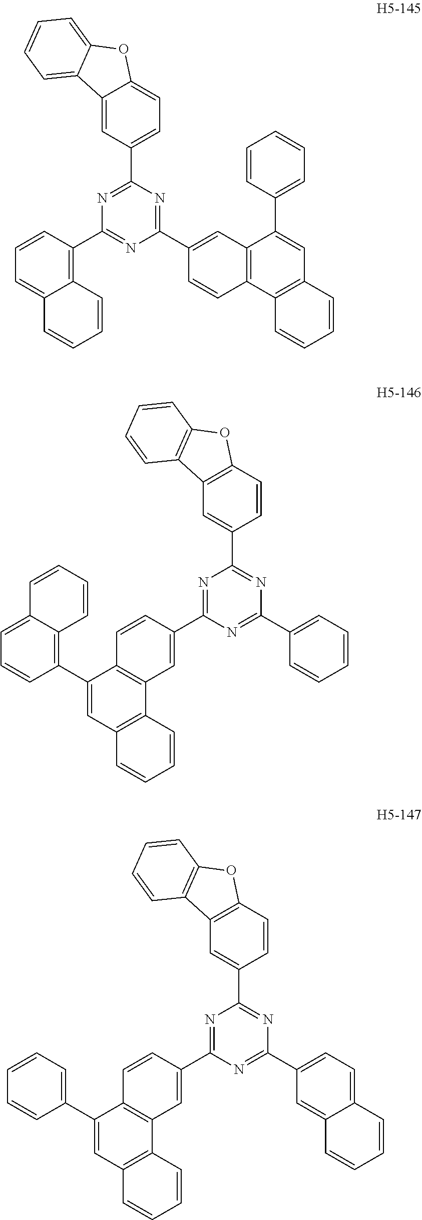 [SMILES: c1ccc(-c2cc3ccccc3c3ccc(-c4nc(-c5ccc6oc7ccccc7c6c5)nc(-c5cccc6ccccc56)n4)cc23)cc1 ; c1ccc(-c2nc(-c3ccc4oc5ccccc5c4c3)nc(-c3ccc4c(-c5cccc6ccccc56)cc5ccccc5c4c3)n2)cc1 ; c1ccc(-c2cc3ccccc3c3cc(-c4nc(-c5ccc6ccccc6c5)nc(-c5ccc6oc7ccccc7c6c5)n4)ccc23)cc1] 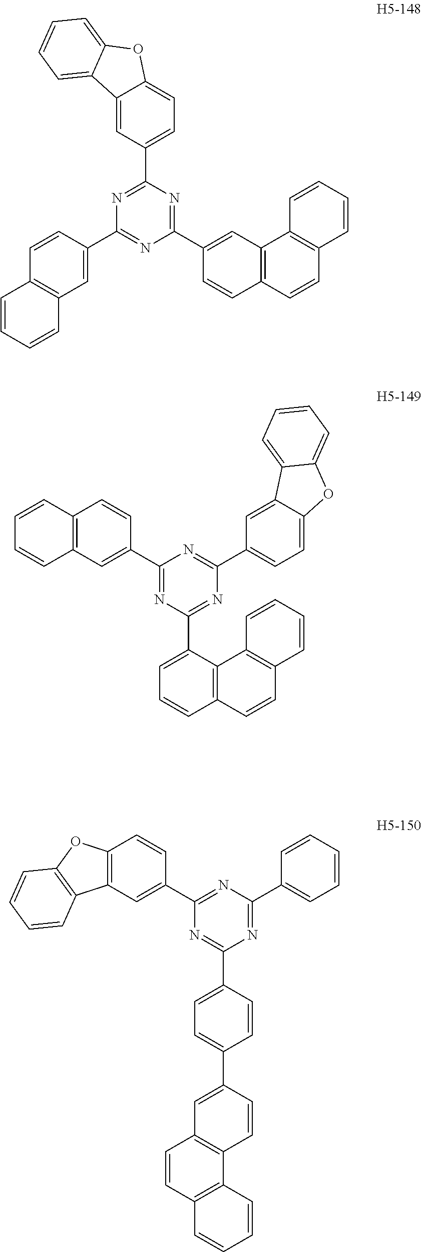 [SMILES: c1ccc2cc(-c3nc(-c4ccc5ccc6ccccc6c5c4)nc(-c4ccc5oc6ccccc6c5c4)n3)ccc2c1 ; c1ccc2cc(-c3nc(-c4ccc5oc6ccccc6c5c4)nc(-c4cccc5ccc6ccccc6c45)n3)ccc2c1 ; c1ccc(-c2nc(-c3ccc(-c4ccc5c(ccc6ccccc65)c4)cc3)nc(-c3ccc4oc5ccccc5c4c3)n2)cc1] 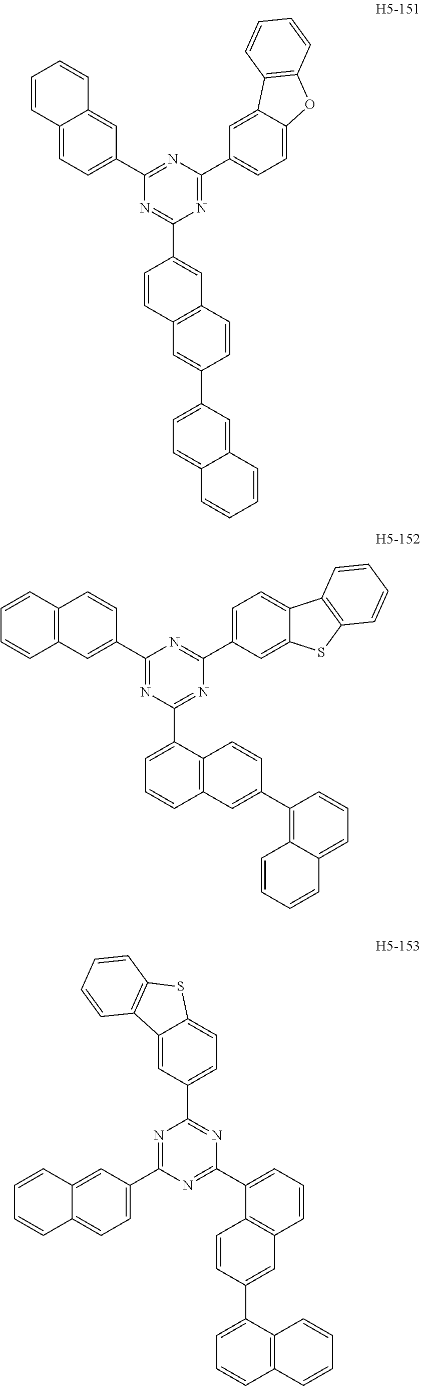 [SMILES: c1ccc2cc(-c3ccc4cc(-c5nc(-c6ccc7ccccc7c6)nc(-c6ccc7oc8ccccc8c7c6)n5)ccc4c3)ccc2c1 ; c1ccc2cc(-c3nc(-c4ccc5c(c4)sc4ccccc45)nc(-c4cccc5cc(-c6cccc7ccccc67)ccc45)n3)ccc2c1 ; c1ccc2cc(-c3nc(-c4ccc5sc6ccccc6c5c4)nc(-c4cccc5cc(-c6cccc7ccccc67)ccc45)n3)ccc2c1] 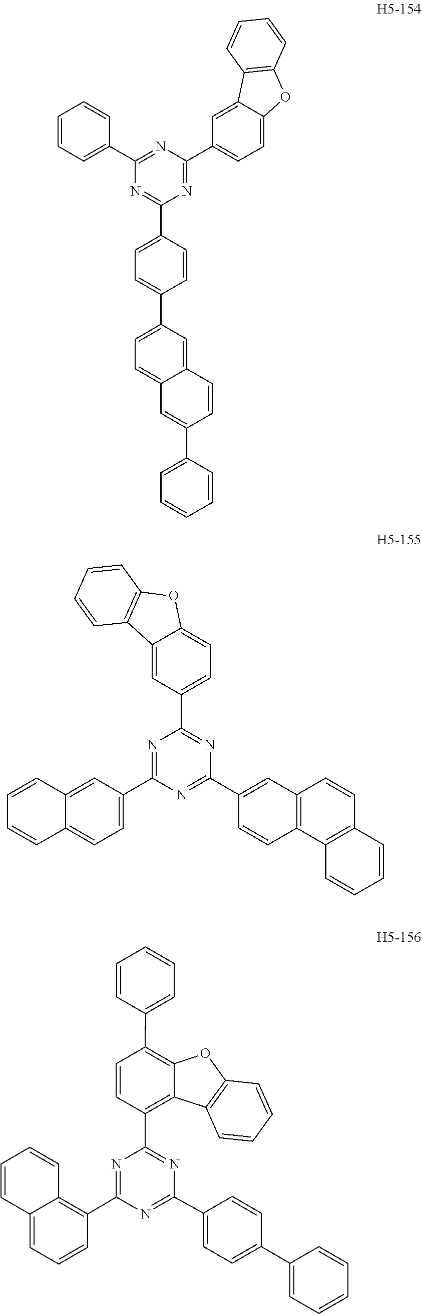 [SMILES: c1ccc(-c2ccc3cc(-c4ccc(-c5nc(-c6ccccc6)nc(-c6ccc7oc8ccccc8c7c6)n5)cc4)ccc3c2)cc1 ; c1ccc2cc(-c3nc(-c4ccc5c(ccc6ccccc65)c4)nc(-c4ccc5oc6ccccc6c5c4)n3)ccc2c1 ; c1ccc(-c2ccc(-c3nc(-c4cccc5ccccc45)nc(-c4ccc(-c5ccccc5)c5oc6ccccc6c45)n3)cc2)cc1] 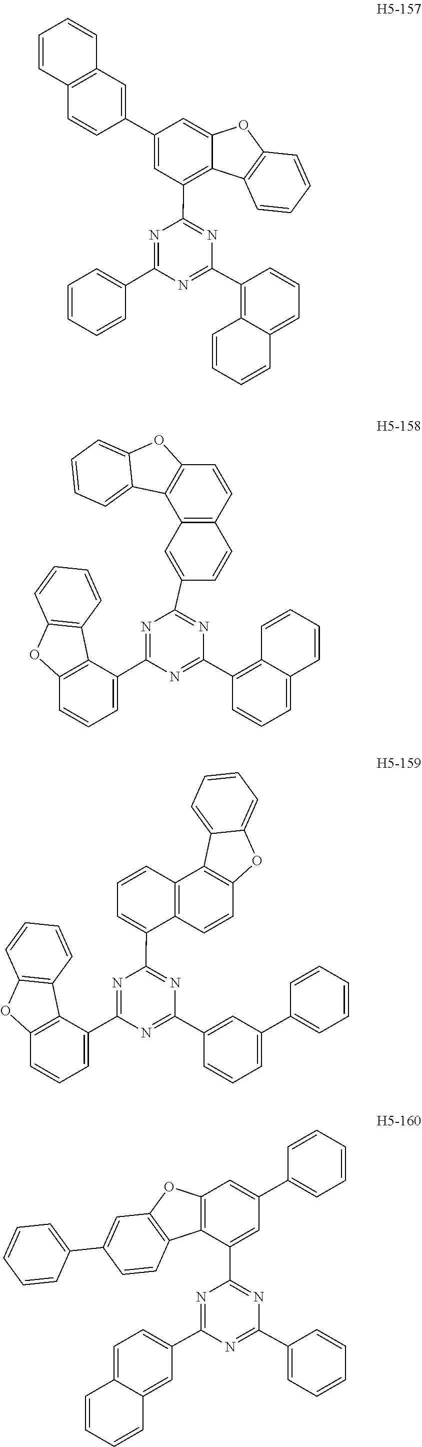 [SMILES: c1ccc(-c2nc(-c3cccc4ccccc34)nc(-c3cc(-c4ccc5ccccc5c4)cc4oc5ccccc5c34)n2)cc1 ; c1ccc2c(-c3nc(-c4ccc5ccc6oc7ccccc7c6c5c4)nc(-c4cccc5oc6ccccc6c45)n3)cccc2c1 ; c1ccc(-c2cccc(-c3nc(-c4cccc5c4ccc4oc6ccccc6c45)nc(-c4cccc5oc6ccccc6c45)n3)c2)cc1 ; c1ccc(-c2ccc3c(c2)oc2cc(-c4ccccc4)cc(-c4nc(-c5ccccc5)nc(-c5ccc6ccccc6c5)n4)c23)cc1] 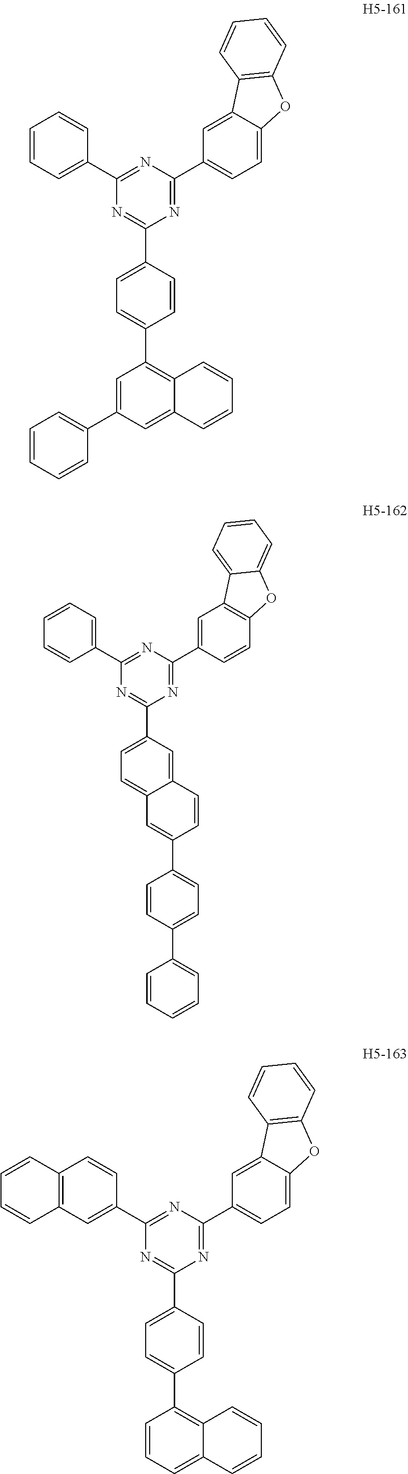 [SMILES: c1ccc(-c2cc(-c3ccc(-c4nc(-c5ccccc5)nc(-c5ccc6oc7ccccc7c6c5)n4)cc3)c3ccccc3c2)cc1 ; c1ccc(-c2ccc(-c3ccc4cc(-c5nc(-c6ccccc6)nc(-c6ccc7oc8ccccc8c7c6)n5)ccc4c3)cc2)cc1 ; c1ccc2cc(-c3nc(-c4ccc(-c5cccc6ccccc56)cc4)nc(-c4ccc5oc6ccccc6c5c4)n3)ccc2c1] 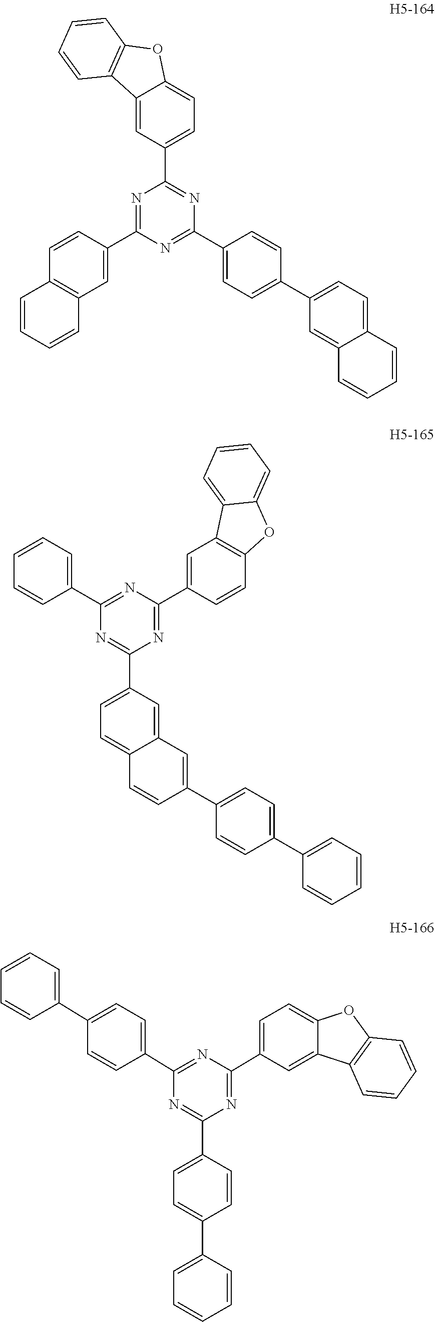 [SMILES: c1ccc2cc(-c3ccc(-c4nc(-c5ccc6ccccc6c5)nc(-c5ccc6oc7ccccc7c6c5)n4)cc3)ccc2c1 ; c1ccc(-c2ccc(-c3ccc4ccc(-c5nc(-c6ccccc6)nc(-c6ccc7oc8ccccc8c7c6)n5)cc4c3)cc2)cc1 ; c1ccc(-c2ccc(-c3nc(-c4ccc(-c5ccccc5)cc4)nc(-c4ccc5oc6ccccc6c5c4)n3)cc2)cc1] 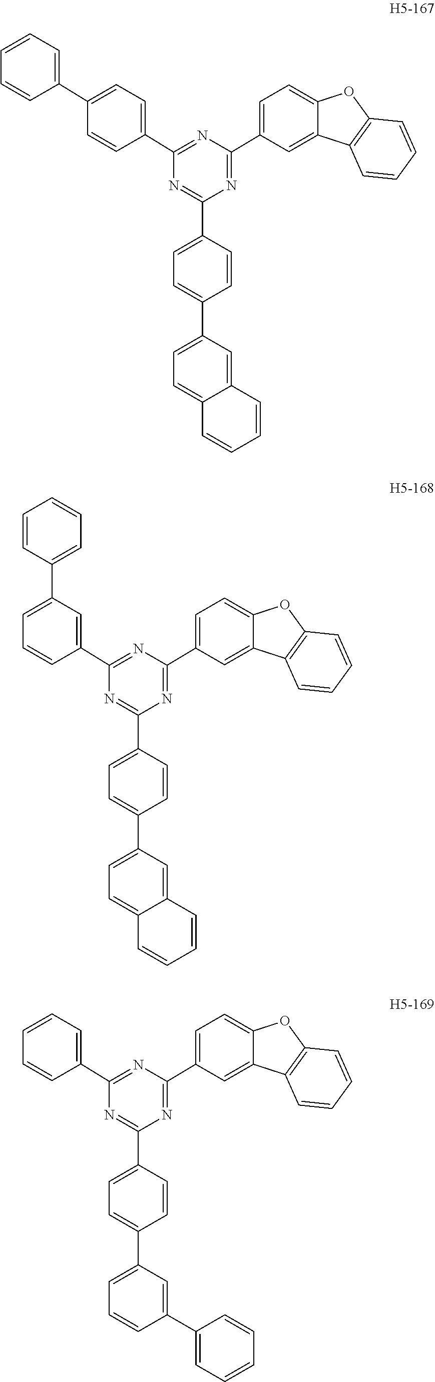 [SMILES: c1ccc(-c2ccc(-c3nc(-c4ccc(-c5ccc6ccccc6c5)cc4)nc(-c4ccc5oc6ccccc6c5c4)n3)cc2)cc1 ; c1ccc(-c2cccc(-c3nc(-c4ccc(-c5ccc6ccccc6c5)cc4)nc(-c4ccc5oc6ccccc6c5c4)n3)c2)cc1 ; c1ccc(-c2cccc(-c3ccc(-c4nc(-c5ccccc5)nc(-c5ccc6oc7ccccc7c6c5)n4)cc3)c2)cc1] 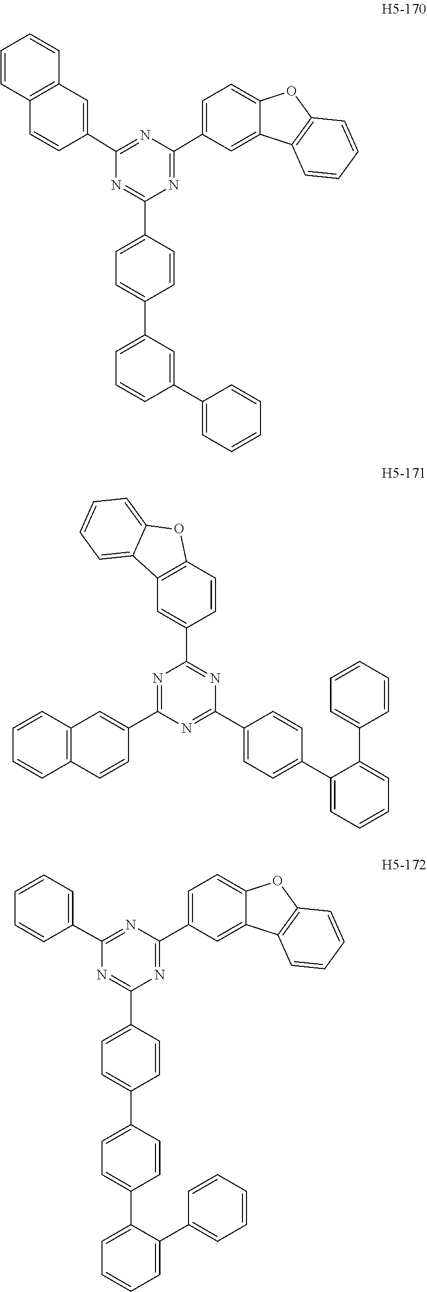 [SMILES: c1ccc(-c2cccc(-c3ccc(-c4nc(-c5ccc6ccccc6c5)nc(-c5ccc6oc7ccccc7c6c5)n4)cc3)c2)cc1 ; c1ccc(-c2ccccc2-c2ccc(-c3nc(-c4ccc5ccccc5c4)nc(-c4ccc5oc6ccccc6c5c4)n3)cc2)cc1 ; c1ccc(-c2nc(-c3ccc(-c4ccc(-c5ccccc5-c5ccccc5)cc4)cc3)nc(-c3ccc4oc5ccccc5c4c3)n2)cc1] 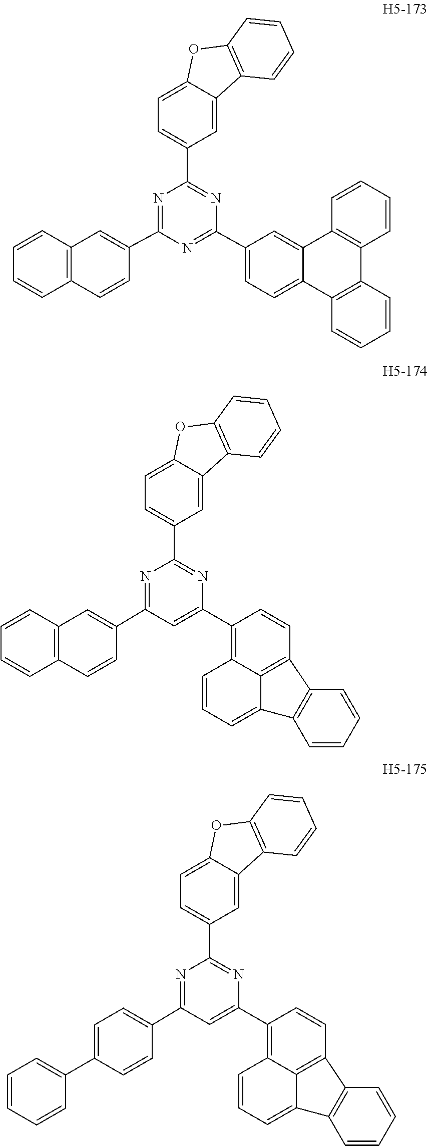 [SMILES: c1ccc2cc(-c3nc(-c4ccc5oc6ccccc6c5c4)nc(-c4ccc5c6ccccc6c6ccccc6c5c4)n3)ccc2c1 ; c1ccc2c(c1)-c1cccc3c(-c4cc(-c5ccc6ccccc6c5)nc(-c5ccc6oc7ccccc7c6c5)n4)ccc-2c13 ; c1ccc(-c2ccc(-c3cc(-c4ccc5c6c(cccc46)-c4ccccc4-5)nc(-c4ccc5oc6ccccc6c5c4)n3)cc2)cc1] 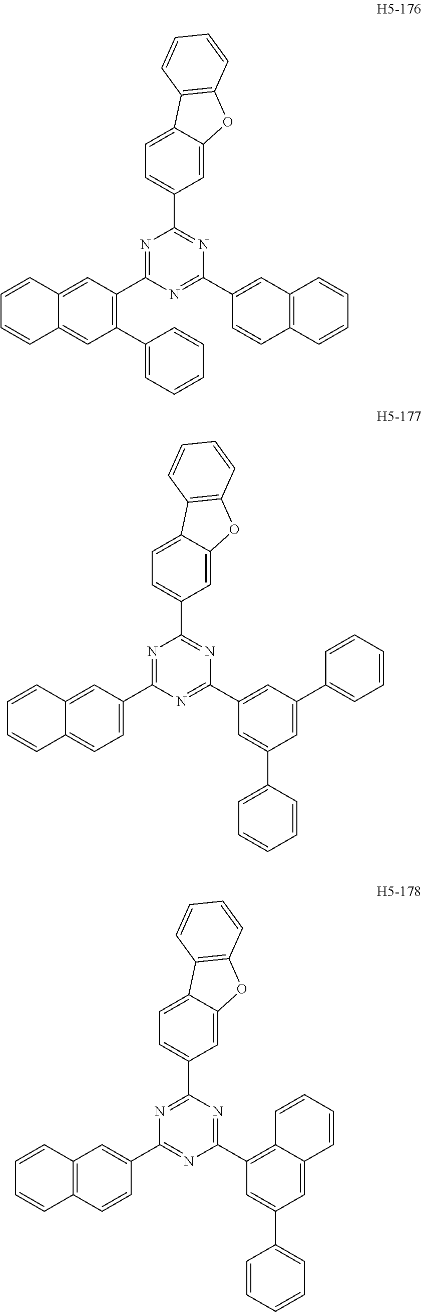 [SMILES: c1ccc(-c2cc3ccccc3cc2-c2nc(-c3ccc4ccccc4c3)nc(-c3ccc4c(c3)oc3ccccc34)n2)cc1 ; c1ccc(-c2cc(-c3ccccc3)cc(-c3nc(-c4ccc5ccccc5c4)nc(-c4ccc5c(c4)oc4ccccc45)n3)c2)cc1 ; c1ccc(-c2cc(-c3nc(-c4ccc5ccccc5c4)nc(-c4ccc5c(c4)oc4ccccc45)n3)c3ccccc3c2)cc1] 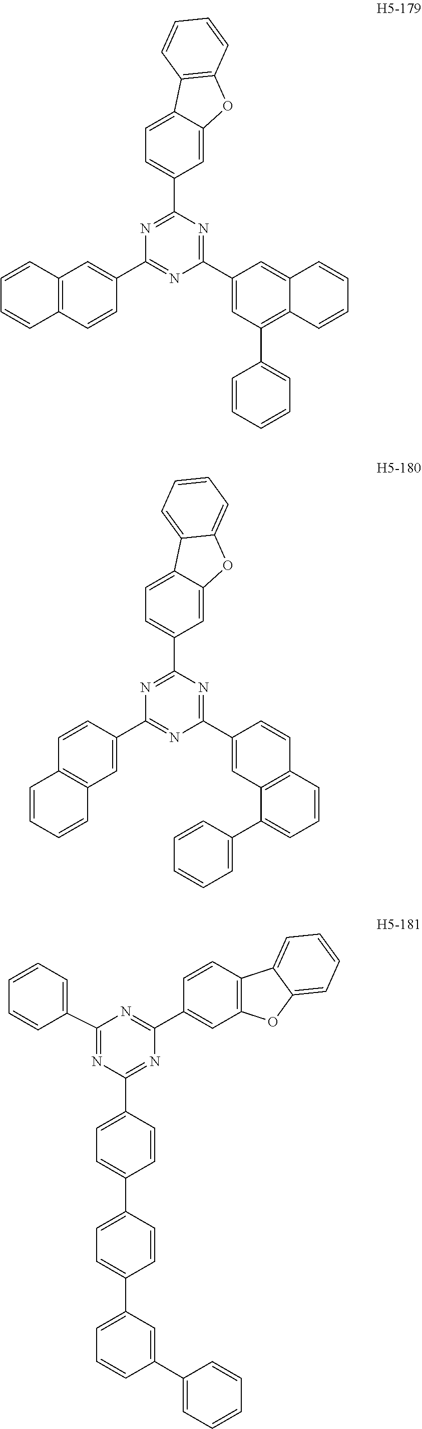 [SMILES: c1ccc(-c2cc(-c3nc(-c4ccc5ccccc5c4)nc(-c4ccc5c(c4)oc4ccccc45)n3)cc3ccccc23)cc1 ; c1ccc(-c2cccc3ccc(-c4nc(-c5ccc6ccccc6c5)nc(-c5ccc6c(c5)oc5ccccc56)n4)cc23)cc1 ; c1ccc(-c2cccc(-c3ccc(-c4ccc(-c5nc(-c6ccccc6)nc(-c6ccc7c(c6)oc6ccccc67)n5)cc4)cc3)c2)cc1] 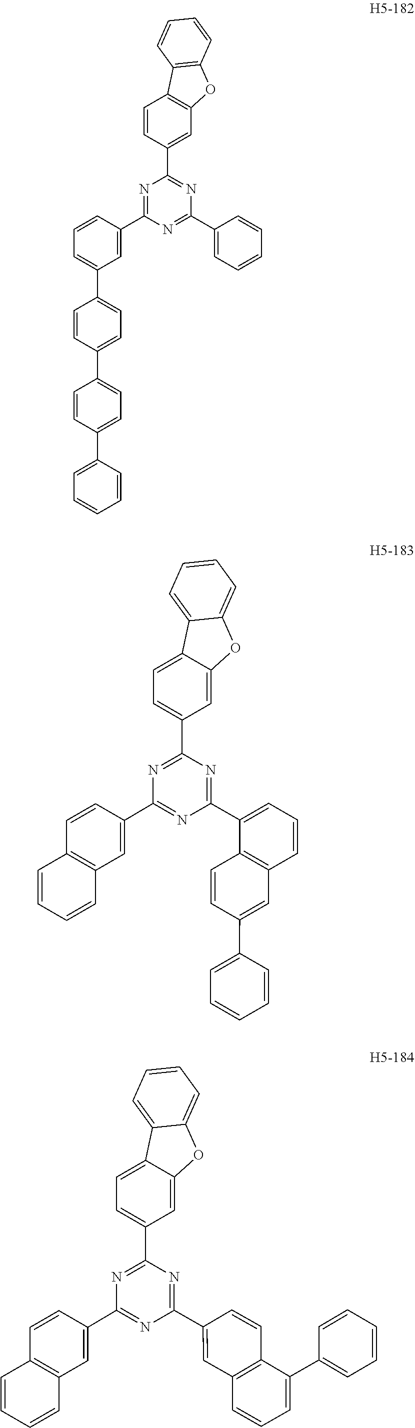 [SMILES: c1ccc(-c2ccc(-c3ccc(-c4cccc(-c5nc(-c6ccccc6)nc(-c6ccc7c(c6)oc6ccccc67)n5)c4)cc3)cc2)cc1 ; c1ccc(-c2ccc3c(-c4nc(-c5ccc6ccccc6c5)nc(-c5ccc6c(c5)oc5ccccc56)n4)cccc3c2)cc1 ; c1ccc(-c2cccc3cc(-c4nc(-c5ccc6ccccc6c5)nc(-c5ccc6c(c5)oc5ccccc56)n4)ccc23)cc1] 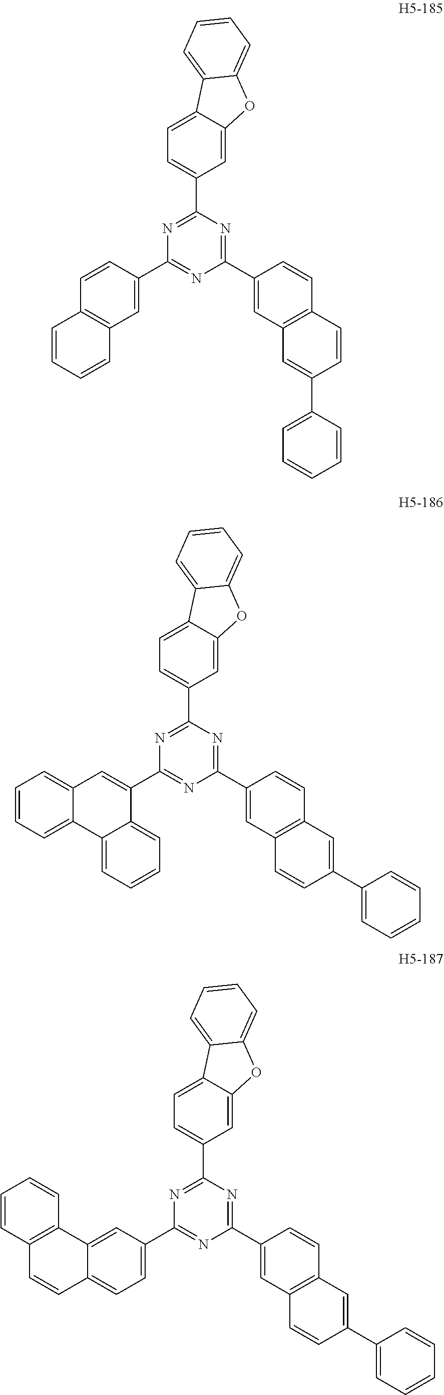 [SMILES: c1ccc(-c2ccc3ccc(-c4nc(-c5ccc6ccccc6c5)nc(-c5ccc6c(c5)oc5ccccc56)n4)cc3c2)cc1 ; c1ccc(-c2ccc3cc(-c4nc(-c5ccc6c(c5)oc5ccccc56)nc(-c5cc6ccccc6c6ccccc56)n4)ccc3c2)cc1 ; c1ccc(-c2ccc3cc(-c4nc(-c5ccc6c(c5)oc5ccccc56)nc(-c5ccc6ccc7ccccc7c6c5)n4)ccc3c2)cc1] 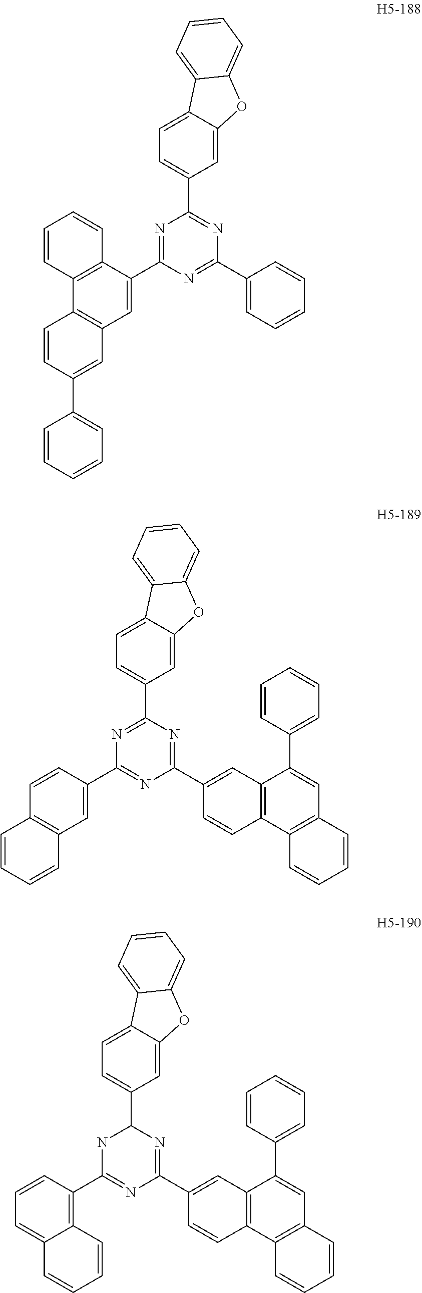 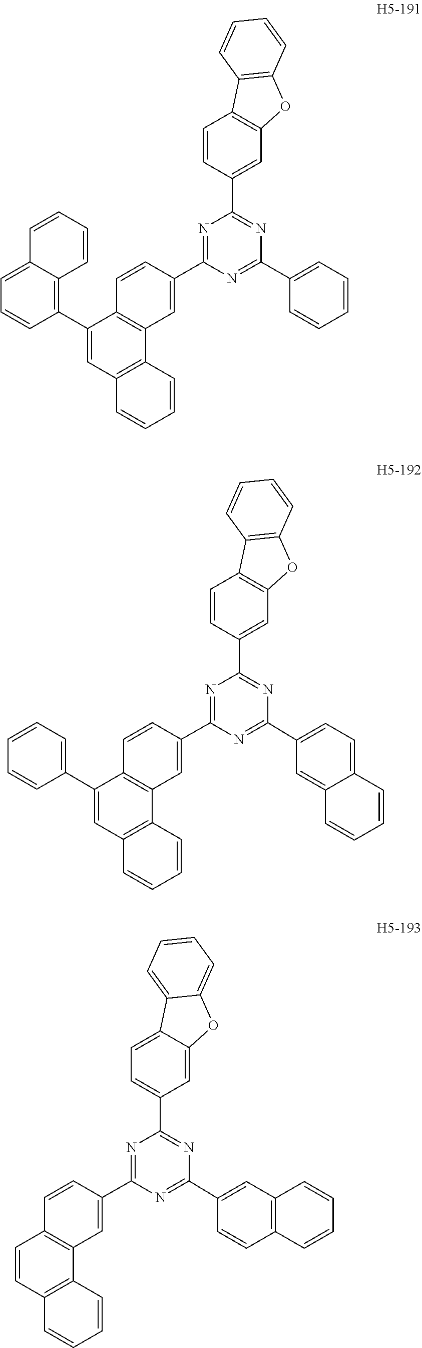 [SMILES: c1ccc(-c2nc(-c3ccc4c(c3)oc3ccccc34)nc(-c3ccc4c(-c5cccc6ccccc56)cc5ccccc5c4c3)n2)cc1 ; c1ccc(-c2cc3ccccc3c3cc(-c4nc(-c5ccc6ccccc6c5)nc(-c5ccc6c(c5)oc5ccccc56)n4)ccc23)cc1 ; c1ccc2cc(-c3nc(-c4ccc5c(c4)oc4ccccc45)nc(-c4ccc5ccc6ccccc6c5c4)n3)ccc2c1] 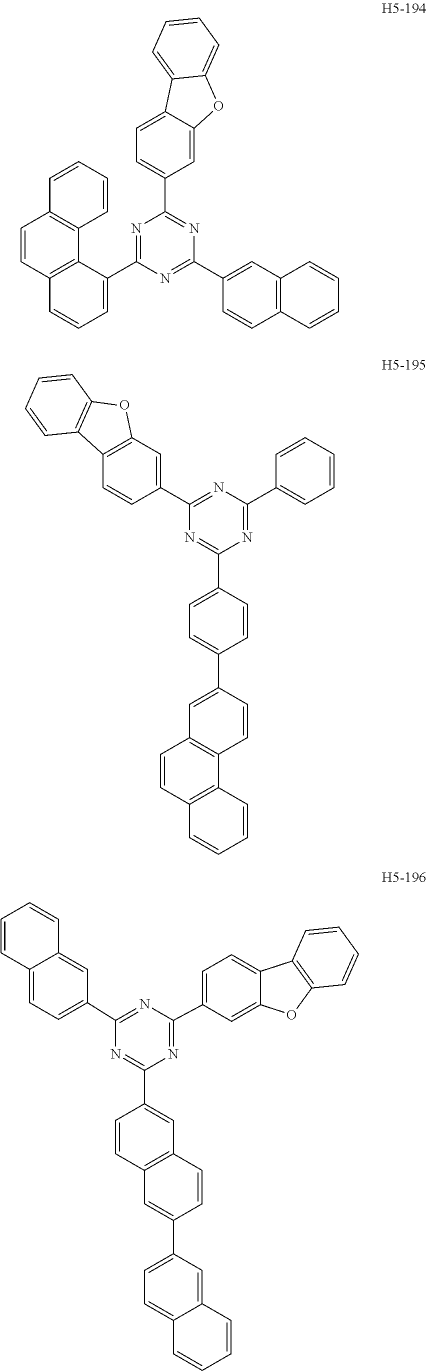 [SMILES: c1ccc2cc(-c3nc(-c4ccc5c(c4)oc4ccccc45)nc(-c4cccc5ccc6ccccc6c45)n3)ccc2c1 ; c1ccc(-c2nc(-c3ccc(-c4ccc5c(ccc6ccccc65)c4)cc3)nc(-c3ccc4c(c3)oc3ccccc34)n2)cc1 ; c1ccc2cc(-c3ccc4cc(-c5nc(-c6ccc7ccccc7c6)nc(-c6ccc7c(c6)oc6ccccc67)n5)ccc4c3)ccc2c1] 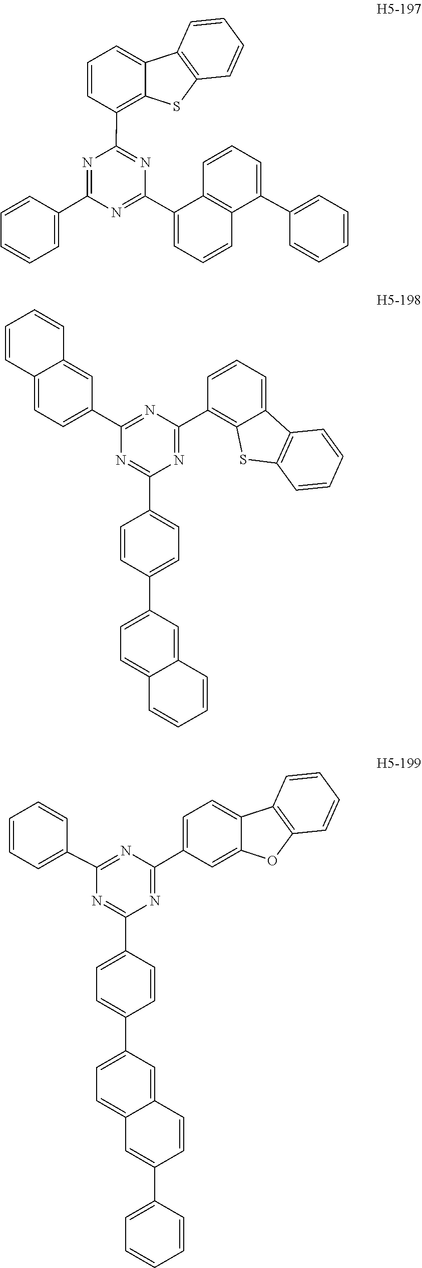 [SMILES: c1ccc(-c2nc(-c3cccc4c(-c5ccccc5)cccc34)nc(-c3cccc4c3sc3ccccc34)n2)cc1 ; c1ccc2cc(-c3ccc(-c4nc(-c5ccc6ccccc6c5)nc(-c5cccc6c5sc5ccccc56)n4)cc3)ccc2c1 ; c1ccc(-c2ccc3cc(-c4ccc(-c5nc(-c6ccccc6)nc(-c6ccc7c(c6)oc6ccccc67)n5)cc4)ccc3c2)cc1] 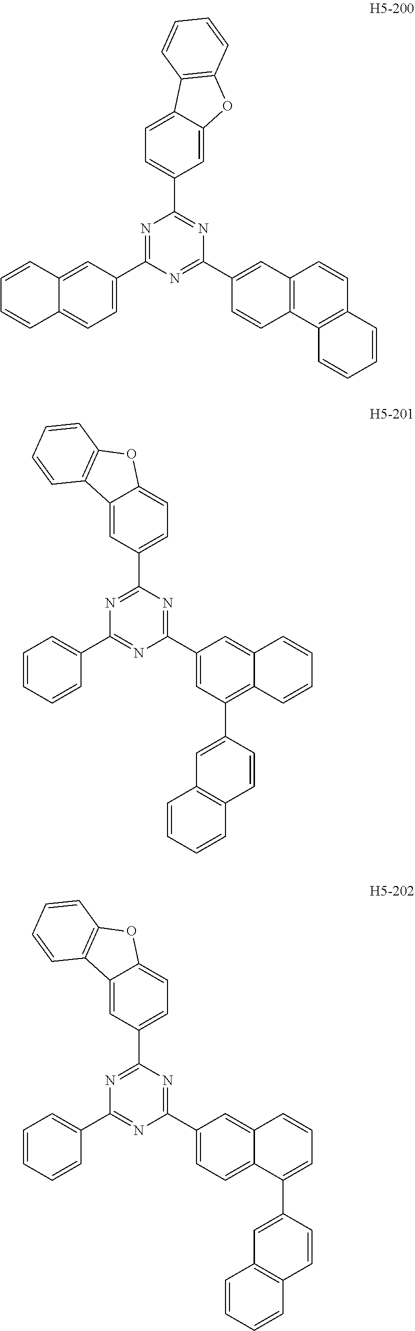 [SMILES: c1ccc2cc(-c3nc(-c4ccc5c(ccc6ccccc65)c4)nc(-c4ccc5c(c4)oc4ccccc45)n3)ccc2c1 ; c1ccc(-c2nc(-c3cc(-c4ccc5ccccc5c4)c4ccccc4c3)nc(-c3ccc4oc5ccccc5c4c3)n2)cc1 ; c1ccc(-c2nc(-c3ccc4c(-c5ccc6ccccc6c5)cccc4c3)nc(-c3ccc4oc5ccccc5c4c3)n2)cc1] 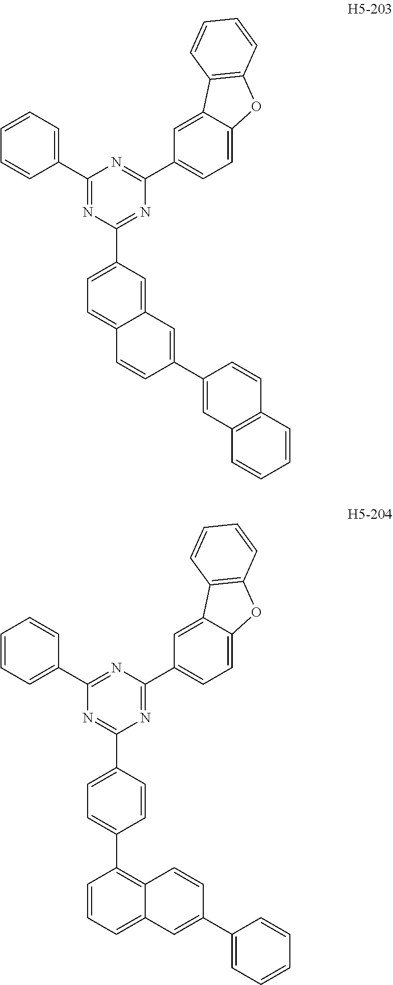 [SMILES: c1ccc(-c2nc(-c3ccc4ccc(-c5ccc6ccccc6c5)cc4c3)nc(-c3ccc4oc5ccccc5c4c3)n2)cc1 ; c1ccc(-c2ccc3c(-c4ccc(-c5nc(-c6ccccc6)nc(-c6ccc7oc8ccccc8c7c6)n5)cc4)cccc3c2)cc1] 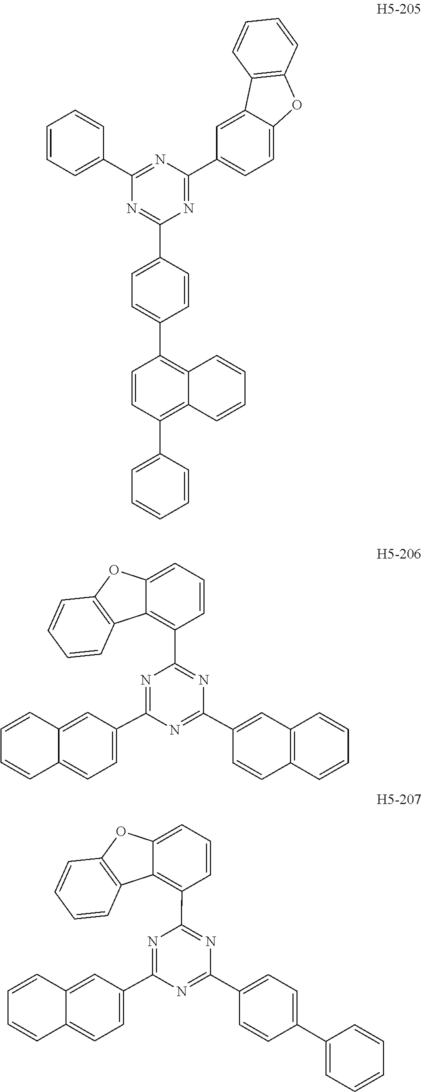 [SMILES: c1ccc(-c2nc(-c3ccc(-c4ccc(-c5ccccc5)c5ccccc45)cc3)nc(-c3ccc4oc5ccccc5c4c3)n2)cc1 ; c1ccc2cc(-c3nc(-c4ccc5ccccc5c4)nc(-c4cccc5oc6ccccc6c45)n3)ccc2c1 ; c1ccc(-c2ccc(-c3nc(-c4ccc5ccccc5c4)nc(-c4cccc5oc6ccccc6c45)n3)cc2)cc1] 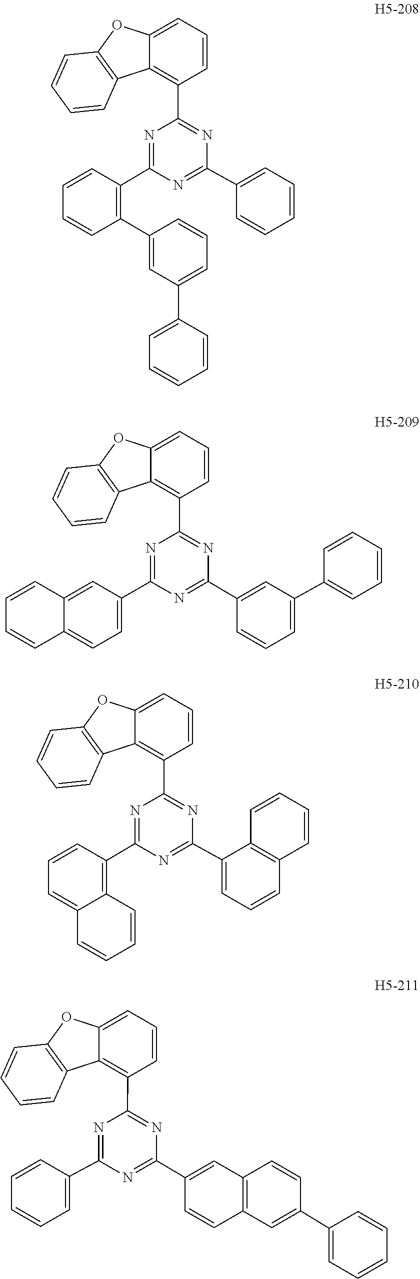 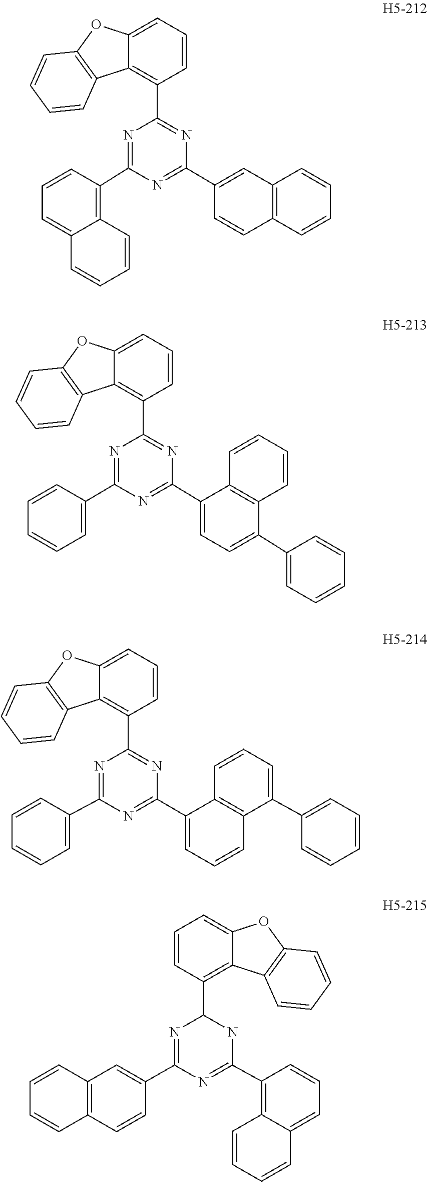 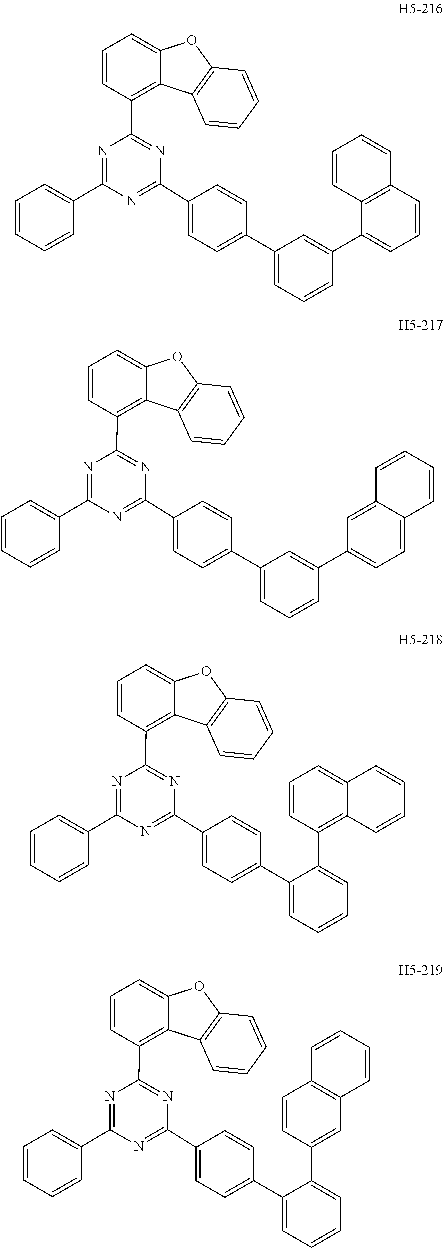 [SMILES: c1ccc(-c2nc(-c3ccc(-c4cccc(-c5cccc6ccccc56)c4)cc3)nc(-c3cccc4oc5ccccc5c34)n2)cc1 ; c1ccc(-c2nc(-c3ccc(-c4cccc(-c5ccc6ccccc6c5)c4)cc3)nc(-c3cccc4oc5ccccc5c34)n2)cc1 ; c1ccc(-c2nc(-c3ccc(-c4ccccc4-c4cccc5ccccc45)cc3)nc(-c3cccc4oc5ccccc5c34)n2)cc1 ; c1ccc(-c2nc(-c3ccc(-c4ccccc4-c4ccc5ccccc5c4)cc3)nc(-c3cccc4oc5ccccc5c34)n2)cc1] 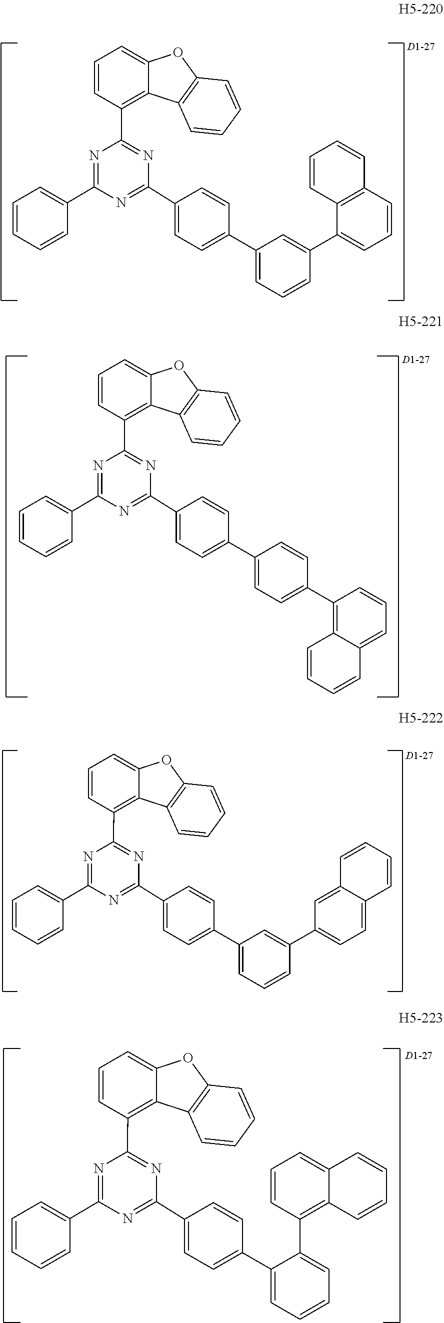 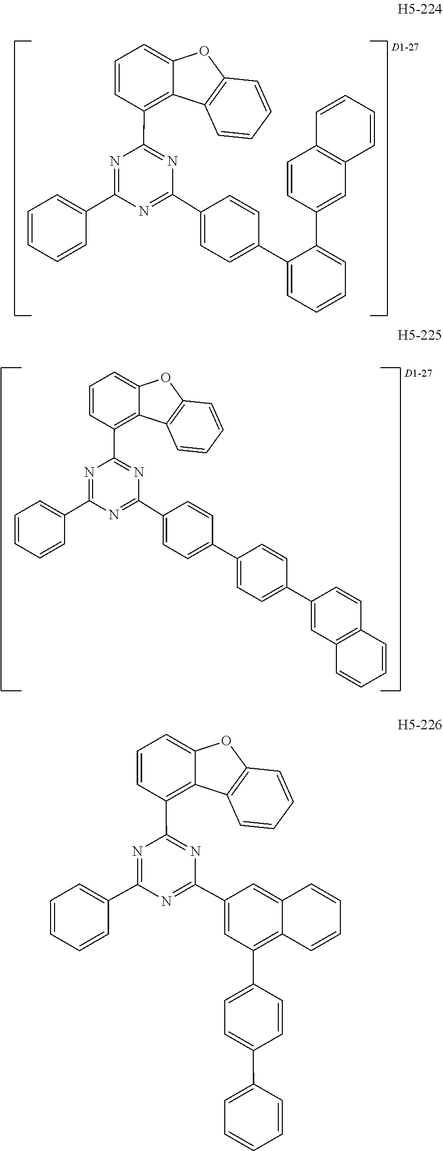 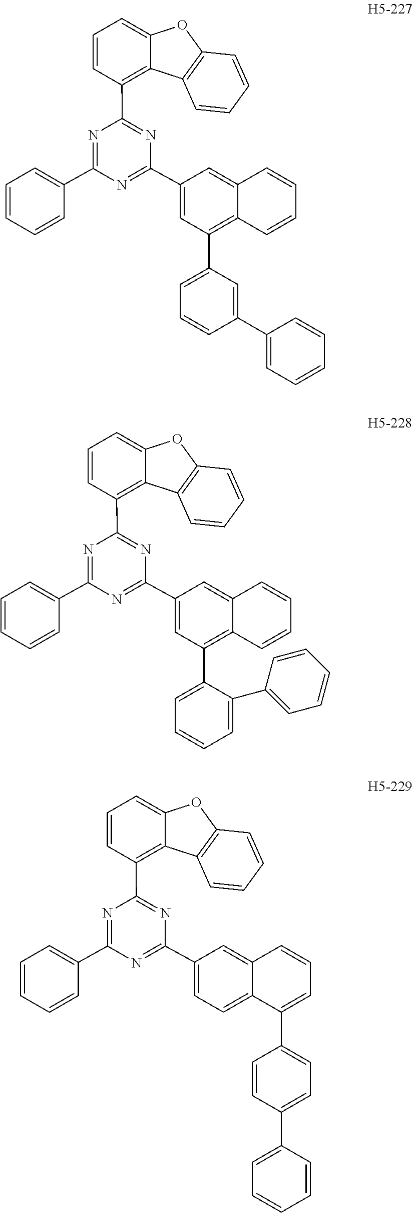 [SMILES: c1ccc(-c2cccc(-c3cc(-c4nc(-c5ccccc5)nc(-c5cccc6oc7ccccc7c56)n4)cc4ccccc34)c2)cc1 ; c1ccc(-c2nc(-c3cc(-c4ccccc4-c4ccccc4)c4ccccc4c3)nc(-c3cccc4oc5ccccc5c34)n2)cc1 ; c1ccc(-c2ccc(-c3cccc4cc(-c5nc(-c6ccccc6)nc(-c6cccc7oc8ccccc8c67)n5)ccc34)cc2)cc1] 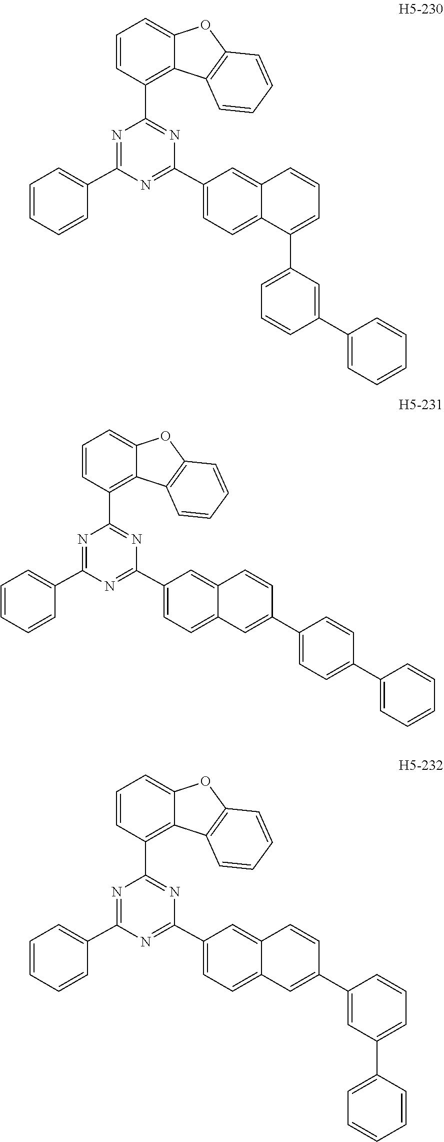 [SMILES: c1ccc(-c2cccc(-c3cccc4cc(-c5nc(-c6ccccc6)nc(-c6cccc7oc8ccccc8c67)n5)ccc34)c2)cc1 ; c1ccc(-c2ccc(-c3ccc4cc(-c5nc(-c6ccccc6)nc(-c6cccc7oc8ccccc8c67)n5)ccc4c3)cc2)cc1 ; c1ccc(-c2cccc(-c3ccc4cc(-c5nc(-c6ccccc6)nc(-c6cccc7oc8ccccc8c67)n5)ccc4c3)c2)cc1] 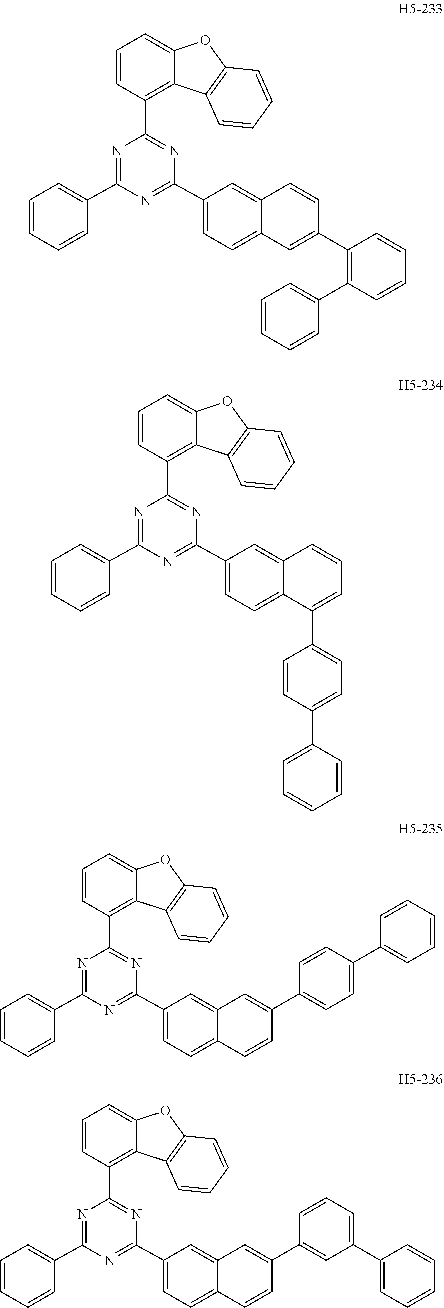 [SMILES: c1ccc(-c2nc(-c3ccc4cc(-c5ccccc5-c5ccccc5)ccc4c3)nc(-c3cccc4oc5ccccc5c34)n2)cc1 ; c1ccc(-c2ccc(-c3cccc4cc(-c5nc(-c6ccccc6)nc(-c6cccc7oc8ccccc8c67)n5)ccc34)cc2)cc1 ; c1ccc(-c2ccc(-c3ccc4ccc(-c5nc(-c6ccccc6)nc(-c6cccc7oc8ccccc8c67)n5)cc4c3)cc2)cc1 ; c1ccc(-c2cccc(-c3ccc4ccc(-c5nc(-c6ccccc6)nc(-c6cccc7oc8ccccc8c67)n5)cc4c3)c2)cc1] 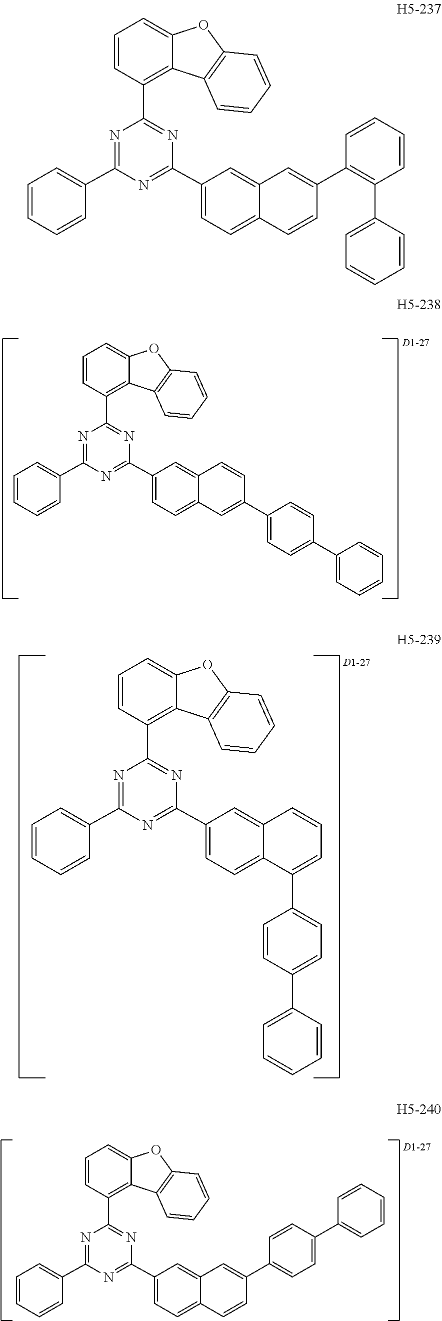 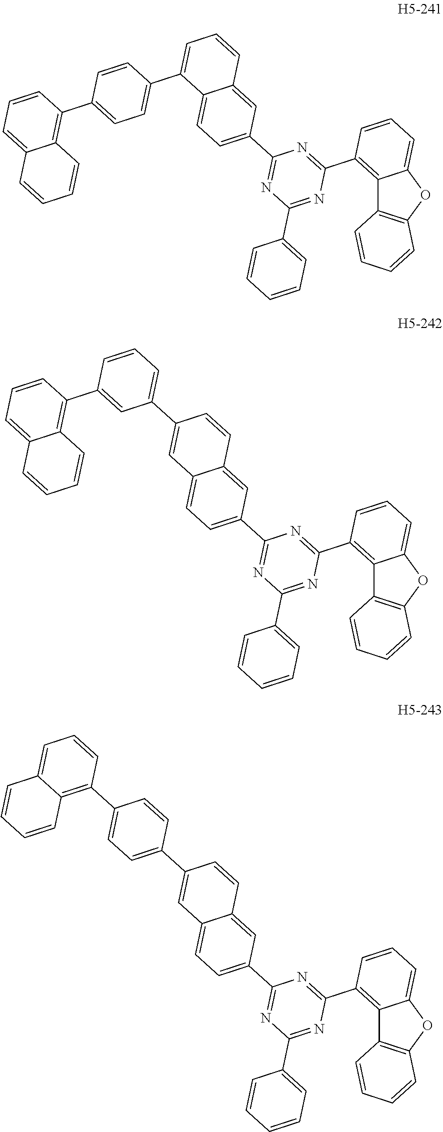 [SMILES: c1ccc(-c2nc(-c3ccc4c(-c5ccc(-c6cccc7ccccc67)cc5)cccc4c3)nc(-c3cccc4oc5ccccc5c34)n2)cc1 ; c1ccc(-c2nc(-c3ccc4cc(-c5cccc(-c6cccc7ccccc67)c5)ccc4c3)nc(-c3cccc4oc5ccccc5c34)n2)cc1 ; c1ccc(-c2nc(-c3ccc4cc(-c5ccc(-c6cccc7ccccc67)cc5)ccc4c3)nc(-c3cccc4oc5ccccc5c34)n2)cc1] 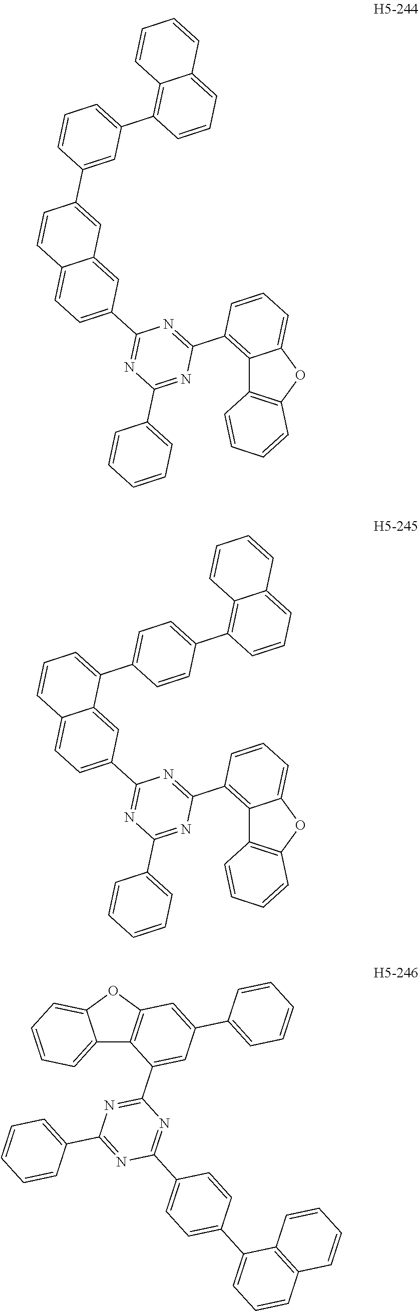 [SMILES: c1ccc(-c2nc(-c3ccc4ccc(-c5cccc(-c6cccc7ccccc67)c5)cc4c3)nc(-c3cccc4oc5ccccc5c34)n2)cc1 ; c1ccc(-c2nc(-c3ccc4cccc(-c5ccc(-c6cccc7ccccc67)cc5)c4c3)nc(-c3cccc4oc5ccccc5c34)n2)cc1 ; c1ccc(-c2cc(-c3nc(-c4ccccc4)nc(-c4ccc(-c5cccc6ccccc56)cc4)n3)c3c(c2)oc2ccccc23)cc1] 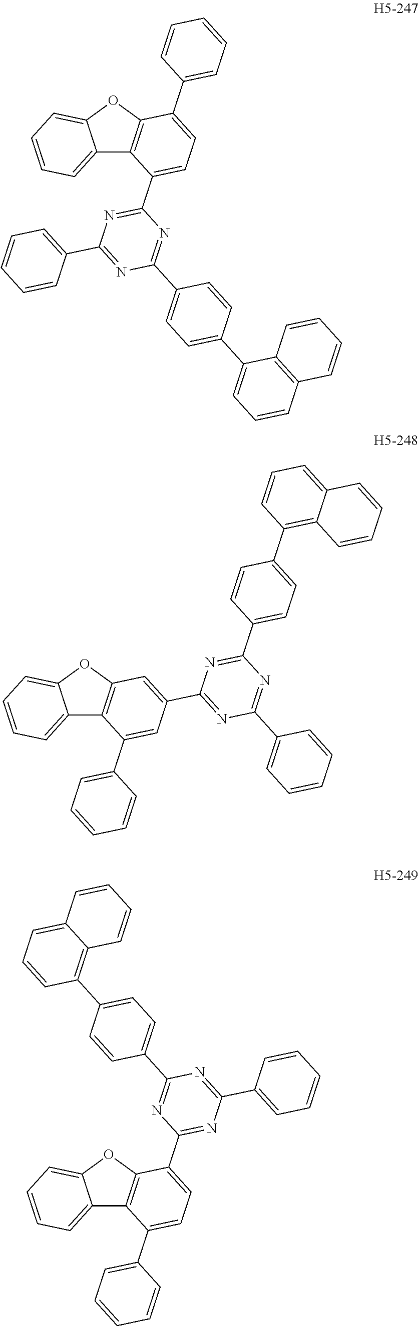 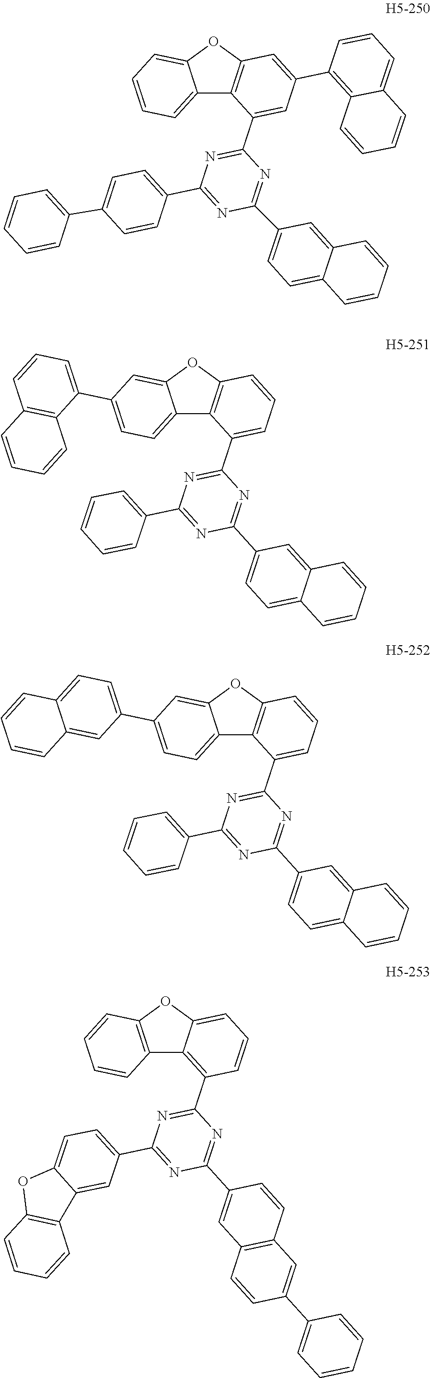 [SMILES: c1ccc(-c2ccc(-c3nc(-c4ccc5ccccc5c4)nc(-c4cc(-c5cccc6ccccc56)cc5oc6ccccc6c45)n3)cc2)cc1 ; c1ccc(-c2nc(-c3ccc4ccccc4c3)nc(-c3cccc4oc5cc(-c6cccc7ccccc67)ccc5c34)n2)cc1 ; c1ccc(-c2nc(-c3ccc4ccccc4c3)nc(-c3cccc4oc5cc(-c6ccc7ccccc7c6)ccc5c34)n2)cc1 ; c1ccc(-c2ccc3cc(-c4nc(-c5ccc6oc7ccccc7c6c5)nc(-c5cccc6oc7ccccc7c56)n4)ccc3c2)cc1] 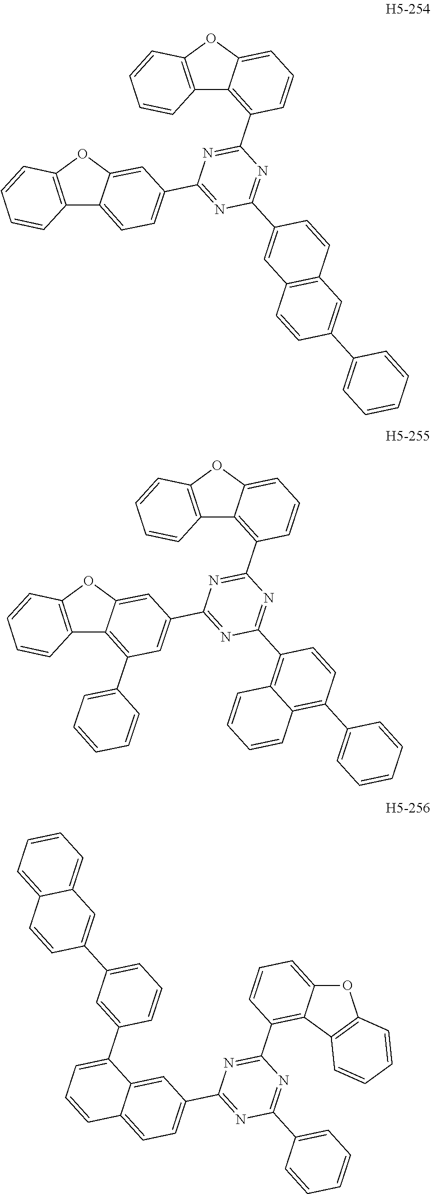 [SMILES: c1ccc(-c2ccc3cc(-c4nc(-c5ccc6c(c5)oc5ccccc56)nc(-c5cccc6oc7ccccc7c56)n4)ccc3c2)cc1 ; c1ccc(-c2ccc(-c3nc(-c4cc(-c5ccccc5)c5c(c4)oc4ccccc45)nc(-c4cccc5oc6ccccc6c45)n3)c3ccccc23)cc1 ; c1ccc(-c2nc(-c3ccc4cccc(-c5cccc(-c6ccc7ccccc7c6)c5)c4c3)nc(-c3cccc4oc5ccccc5c34)n2)cc1] 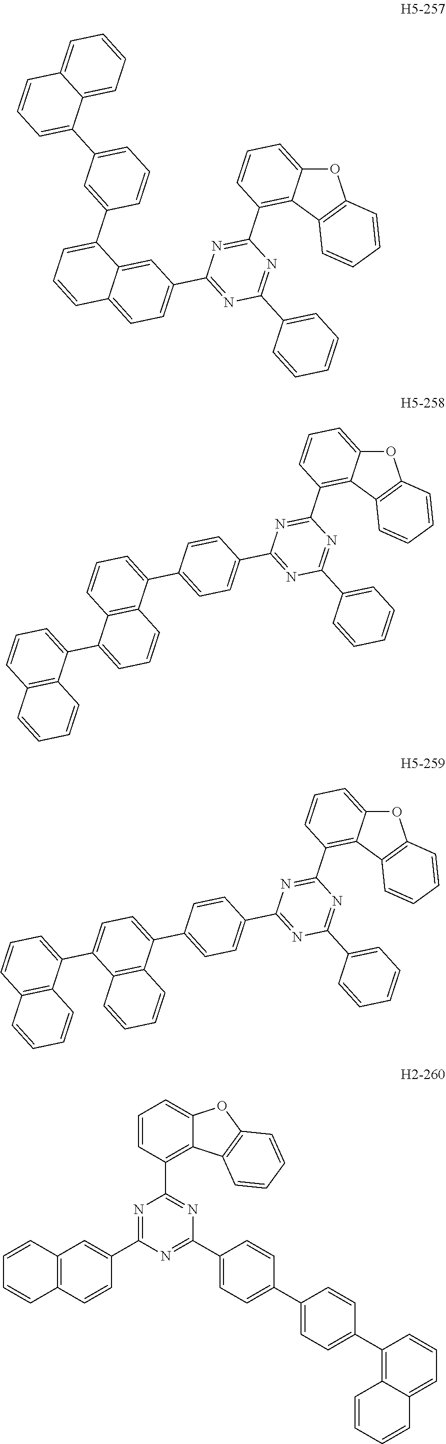 [SMILES: c1ccc(-c2nc(-c3ccc4cccc(-c5cccc(-c6cccc7ccccc67)c5)c4c3)nc(-c3cccc4oc5ccccc5c34)n2)cc1 ; c1ccc(-c2nc(-c3ccc(-c4cccc5c(-c6cccc7ccccc67)cccc45)cc3)nc(-c3cccc4oc5ccccc5c34)n2)cc1 ; c1ccc(-c2nc(-c3ccc(-c4ccc(-c5cccc6ccccc56)c5ccccc45)cc3)nc(-c3cccc4oc5ccccc5c34)n2)cc1 ; c1ccc2cc(-c3nc(-c4ccc(-c5ccc(-c6cccc7ccccc67)cc5)cc4)nc(-c4cccc5oc6ccccc6c45)n3)ccc2c1] 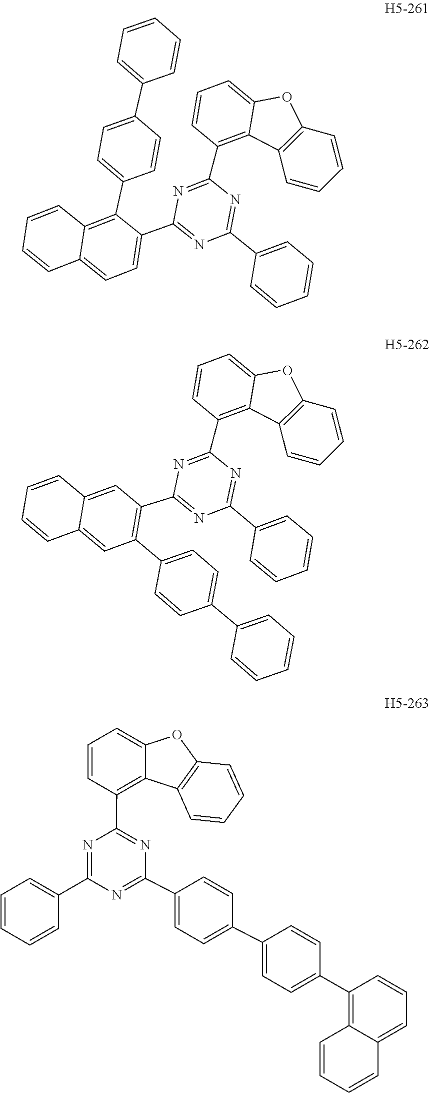 [SMILES: c1ccc(-c2ccc(-c3c(-c4nc(-c5ccccc5)nc(-c5cccc6oc7ccccc7c56)n4)ccc4ccccc34)cc2)cc1 ; c1ccc(-c2ccc(-c3cc4ccccc4cc3-c3nc(-c4ccccc4)nc(-c4cccc5oc6ccccc6c45)n3)cc2)cc1 ; c1ccc(-c2nc(-c3ccc(-c4ccc(-c5cccc6ccccc56)cc4)cc3)nc(-c3cccc4oc5ccccc5c34)n2)cc1] 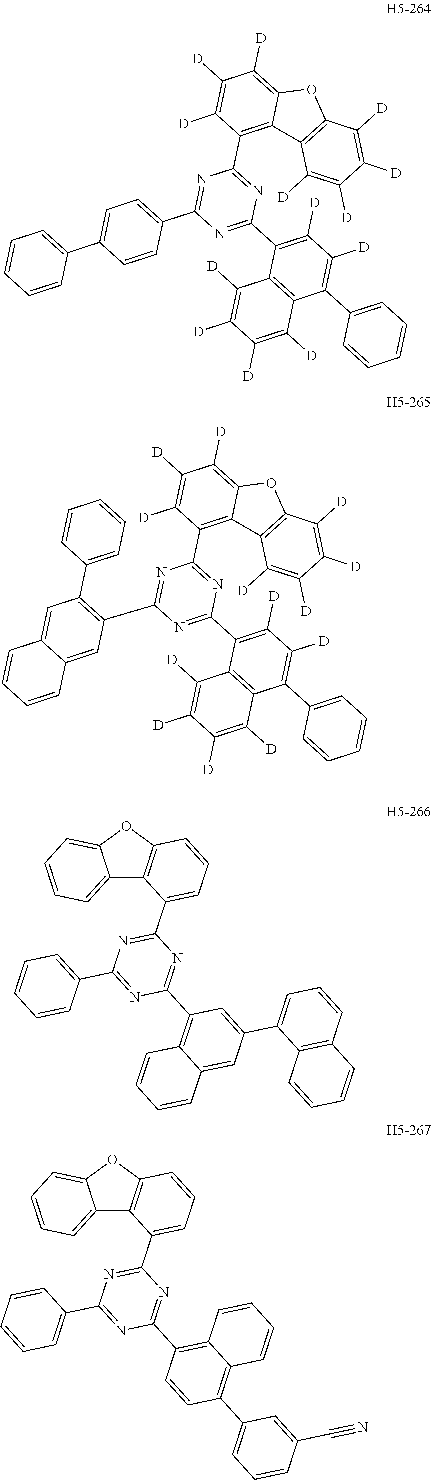 [SMILES: [2H]c1c([2H])c([2H])c2c(oc3c([2H])c([2H])c([2H])c(-c4nc(-c5ccc(-c6ccccc6)cc5)nc(-c5c([2H])c([2H])c(-c6ccccc6)c6c([2H])c([2H])c([2H])c([2H])c56)n4)c32)c1[2H] ; [2H]c1c([2H])c([2H])c2c(oc3c([2H])c([2H])c([2H])c(-c4nc(-c5cc6ccccc6cc5-c5ccccc5)nc(-c5c([2H])c([2H])c(-c6ccccc6)c6c([2H])c([2H])c([2H])c([2H])c56)n4)c32)c1[2H] ; c1ccc(-c2nc(-c3cc(-c4cccc5ccccc45)cc4ccccc34)nc(-c3cccc4oc5ccccc5c34)n2)cc1 ; N#Cc1cccc(-c2ccc(-c3nc(-c4ccccc4)nc(-c4cccc5oc6ccccc6c45)n3)c3ccccc23)c1] 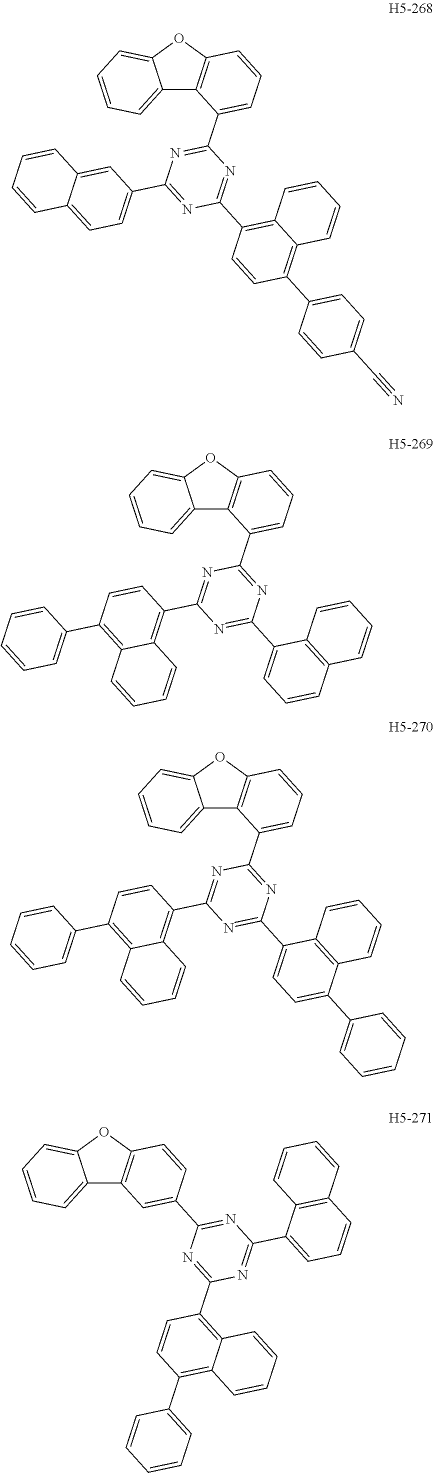 [SMILES: N#Cc1ccc(-c2ccc(-c3nc(-c4ccc5ccccc5c4)nc(-c4cccc5oc6ccccc6c45)n3)c3ccccc23)cc1 ; c1ccc(-c2ccc(-c3nc(-c4cccc5ccccc45)nc(-c4cccc5oc6ccccc6c45)n3)c3ccccc23)cc1 ; c1ccc(-c2ccc(-c3nc(-c4ccc(-c5ccccc5)c5ccccc45)nc(-c4cccc5oc6ccccc6c45)n3)c3ccccc23)cc1 ; c1ccc(-c2ccc(-c3nc(-c4ccc5oc6ccccc6c5c4)nc(-c4cccc5ccccc45)n3)c3ccccc23)cc1] 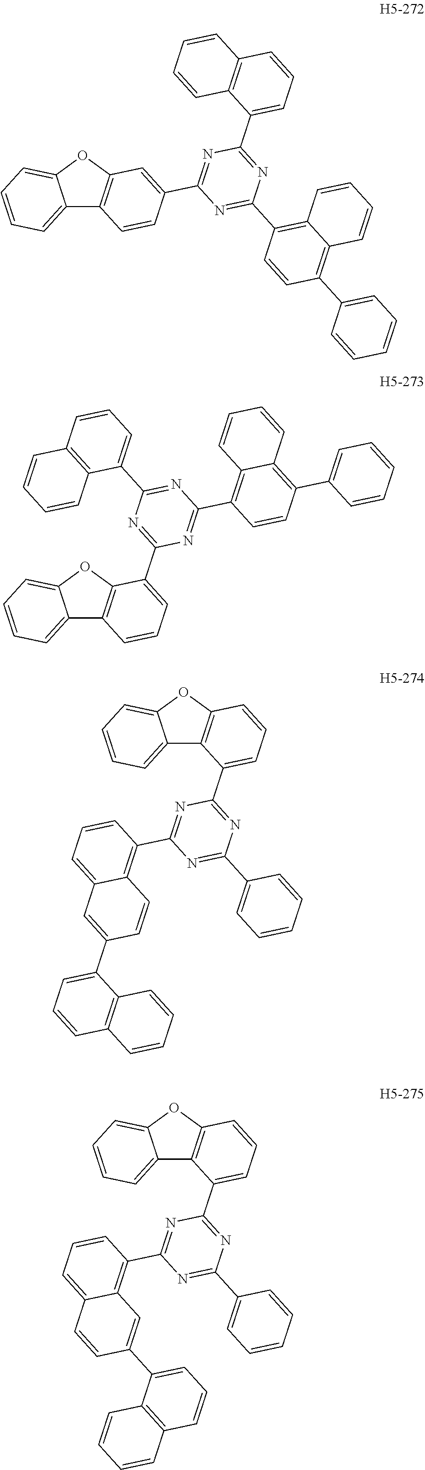 [SMILES: c1ccc(-c2ccc(-c3nc(-c4ccc5c(c4)oc4ccccc45)nc(-c4cccc5ccccc45)n3)c3ccccc23)cc1 ; c1ccc(-c2ccc(-c3nc(-c4cccc5ccccc45)nc(-c4cccc5c4oc4ccccc45)n3)c3ccccc23)cc1 ; c1ccc(-c2nc(-c3cccc4cc(-c5cccc6ccccc56)ccc34)nc(-c3cccc4oc5ccccc5c34)n2)cc1 ; c1ccc(-c2nc(-c3cccc4ccc(-c5cccc6ccccc56)cc34)nc(-c3cccc4oc5ccccc5c34)n2)cc1] 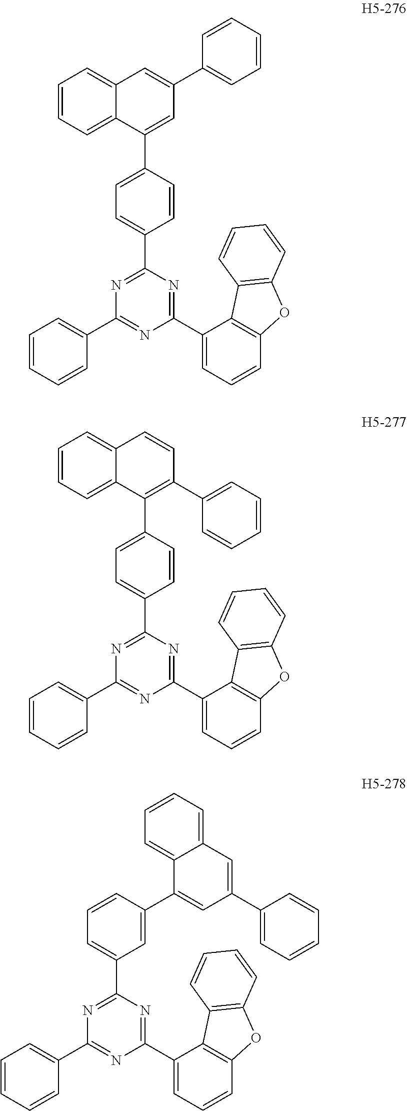 [SMILES: c1ccc(-c2cc(-c3ccc(-c4nc(-c5ccccc5)nc(-c5cccc6oc7ccccc7c56)n4)cc3)c3ccccc3c2)cc1 ; c1ccc(-c2nc(-c3ccc(-c4c(-c5ccccc5)ccc5ccccc45)cc3)nc(-c3cccc4oc5ccccc5c34)n2)cc1 ; c1ccc(-c2cc(-c3cccc(-c4nc(-c5ccccc5)nc(-c5cccc6oc7ccccc7c56)n4)c3)c3ccccc3c2)cc1] 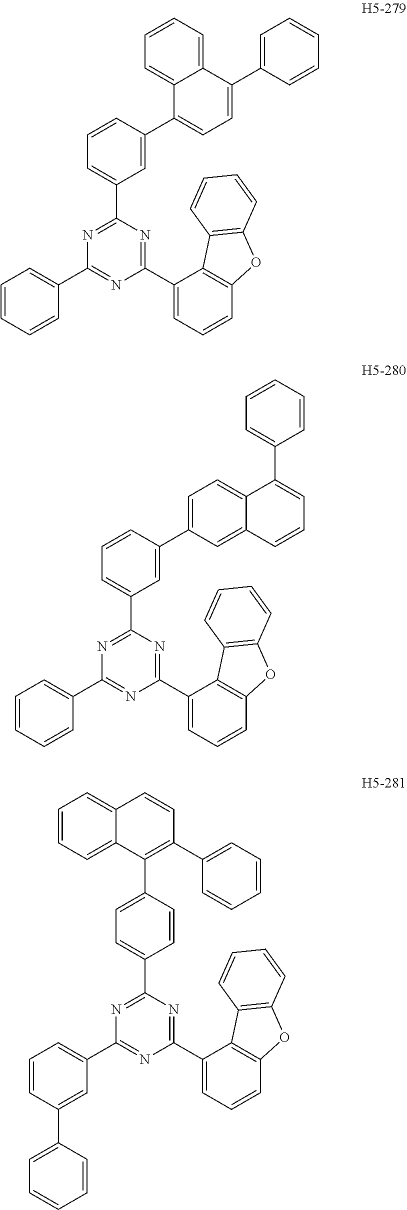 [SMILES: c1ccc(-c2nc(-c3cccc(-c4ccc(-c5ccccc5)c5ccccc45)c3)nc(-c3cccc4oc5ccccc5c34)n2)cc1 ; c1ccc(-c2nc(-c3cccc(-c4ccc5c(-c6ccccc6)cccc5c4)c3)nc(-c3cccc4oc5ccccc5c34)n2)cc1 ; c1ccc(-c2cccc(-c3nc(-c4ccc(-c5c(-c6ccccc6)ccc6ccccc56)cc4)nc(-c4cccc5oc6ccccc6c45)n3)c2)cc1] 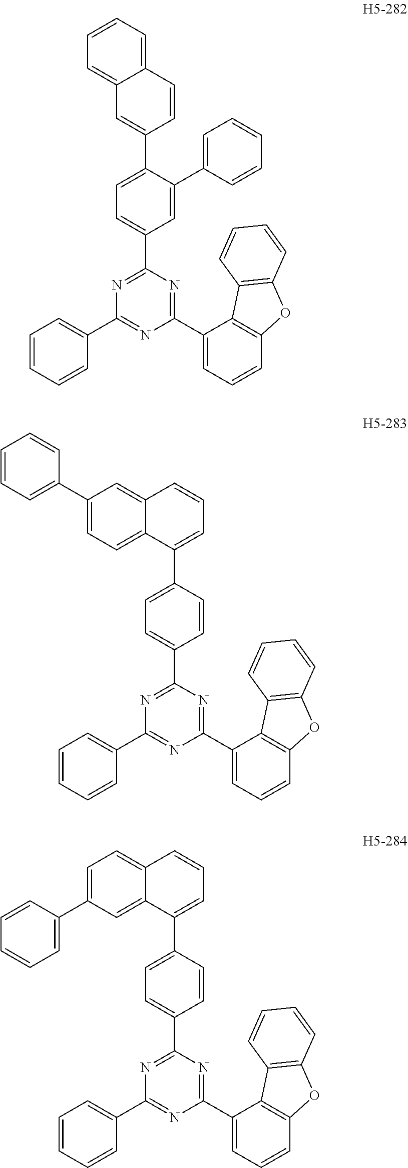 [SMILES: c1ccc(-c2nc(-c3ccc(-c4ccc5ccccc5c4)c(-c4ccccc4)c3)nc(-c3cccc4oc5ccccc5c34)n2)cc1 ; c1ccc(-c2ccc3c(-c4ccc(-c5nc(-c6ccccc6)nc(-c6cccc7oc8ccccc8c67)n5)cc4)cccc3c2)cc1 ; c1ccc(-c2ccc3cccc(-c4ccc(-c5nc(-c6ccccc6)nc(-c6cccc7oc8ccccc8c67)n5)cc4)c3c2)cc1] 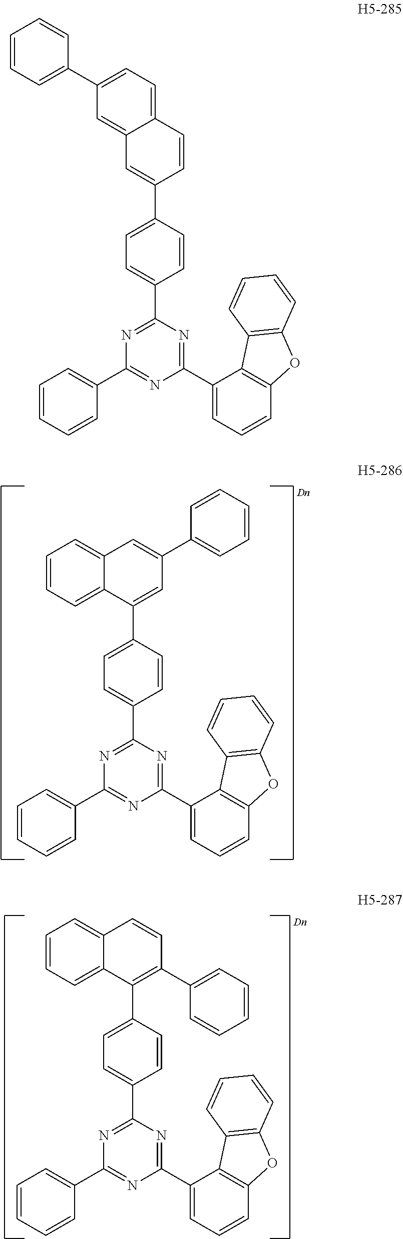 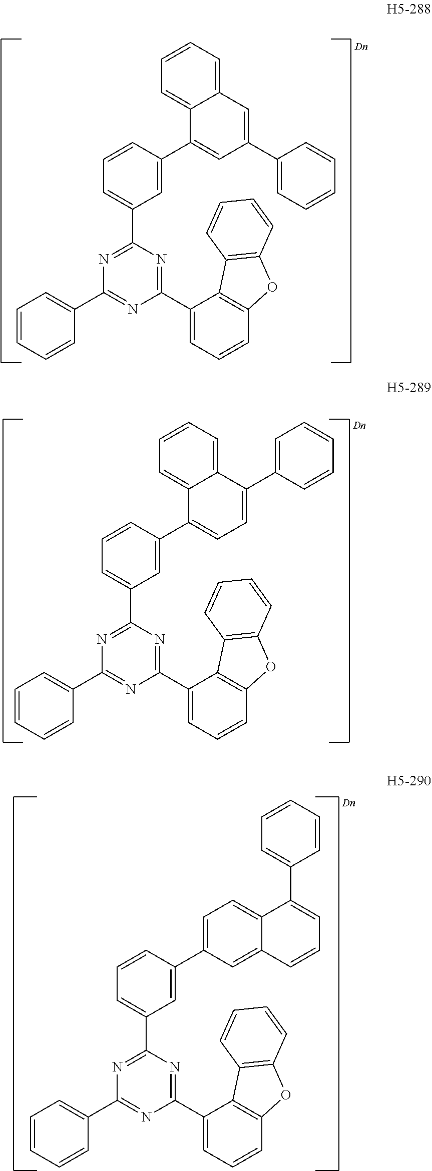 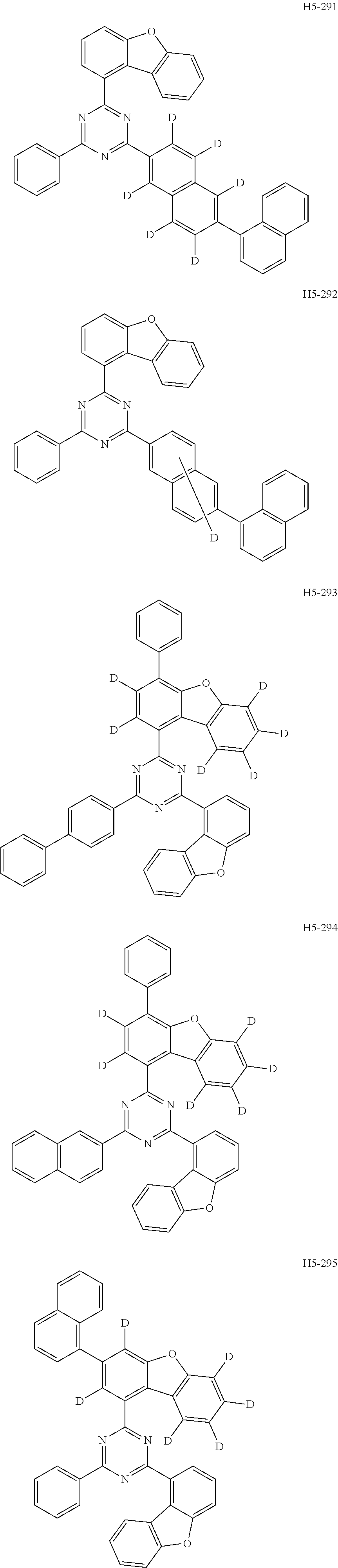 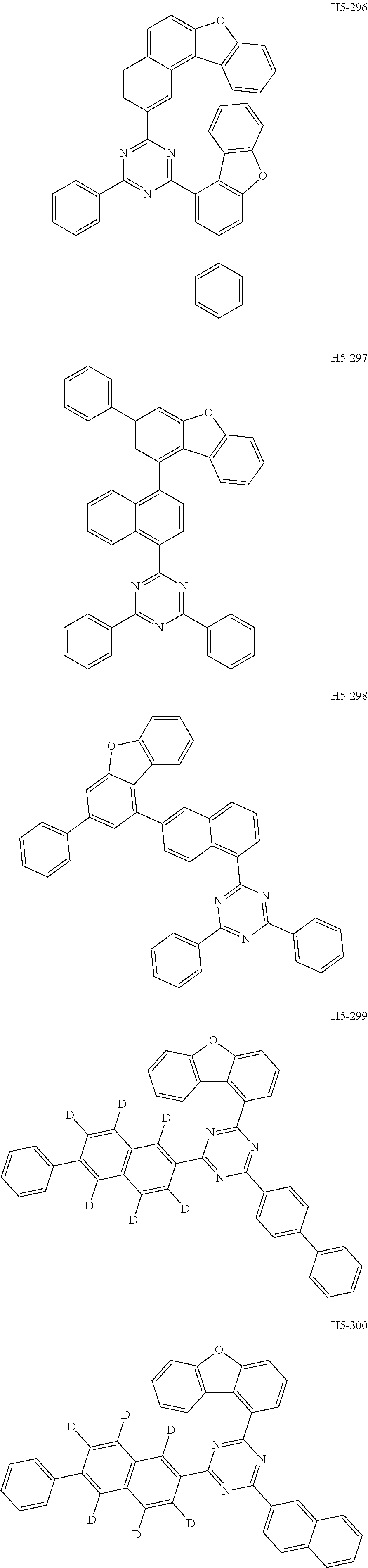 [SMILES: c1ccc(-c2cc(-c3nc(-c4ccccc4)nc(-c4ccc5ccc6oc7ccccc7c6c5c4)n3)c3c(c2)oc2ccccc23)cc1 ; c1ccc(-c2cc(-c3ccc(-c4nc(-c5ccccc5)nc(-c5ccccc5)n4)c4ccccc34)c3c(c2)oc2ccccc23)cc1 ; c1ccc(-c2cc(-c3ccc4c(-c5nc(-c6ccccc6)nc(-c6ccccc6)n5)cccc4c3)c3c(c2)oc2ccccc23)cc1 ; [2H]c1c(-c2nc(-c3ccc(-c4ccccc4)cc3)nc(-c3cccc4oc5ccccc5c34)n2)c([2H])c2c([2H])c([2H])c(-c3ccccc3)c([2H])c2c1[2H] ; [2H]c1c(-c2nc(-c3ccc4ccccc4c3)nc(-c3cccc4oc5ccccc5c34)n2)c([2H])c2c([2H])c([2H])c(-c3ccccc3)c([2H])c2c1[2H]] 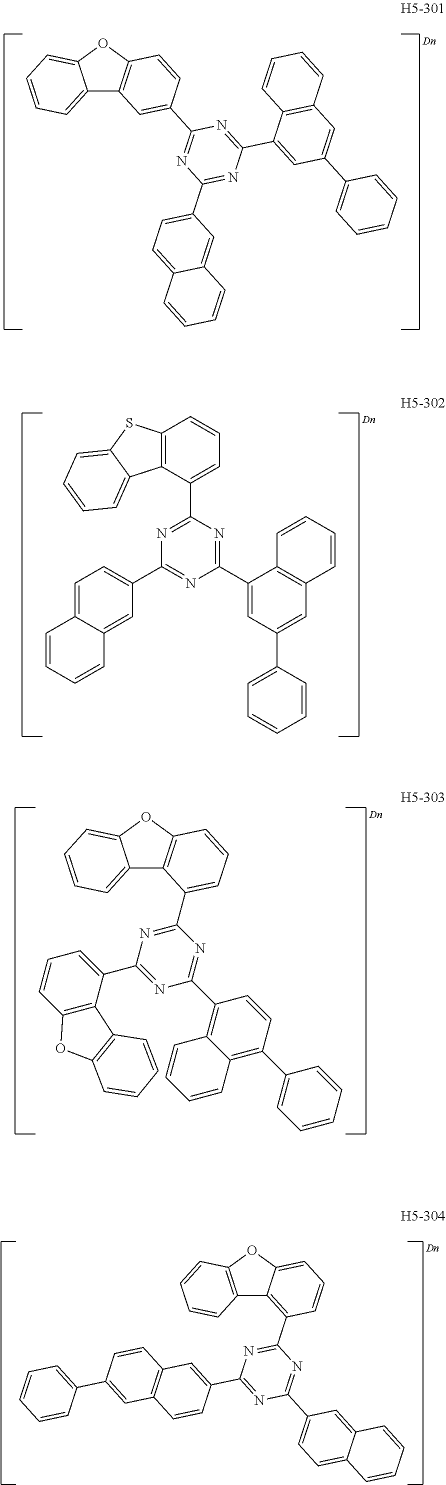 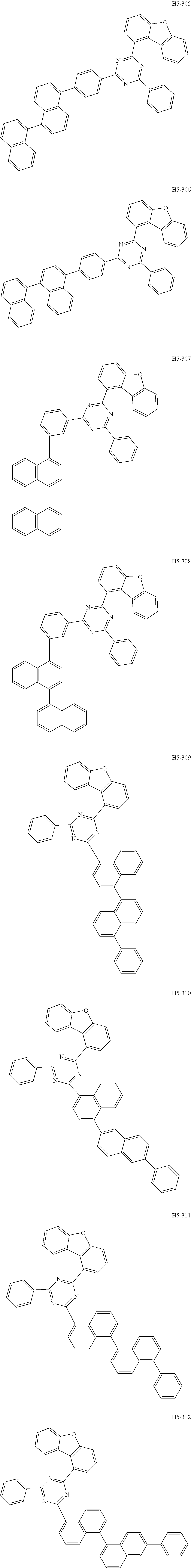 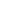 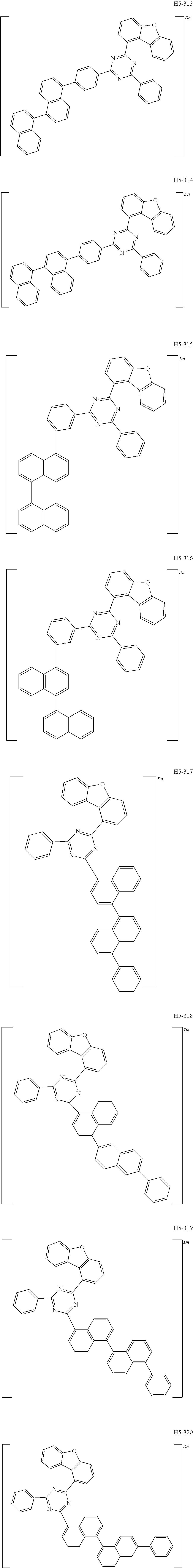 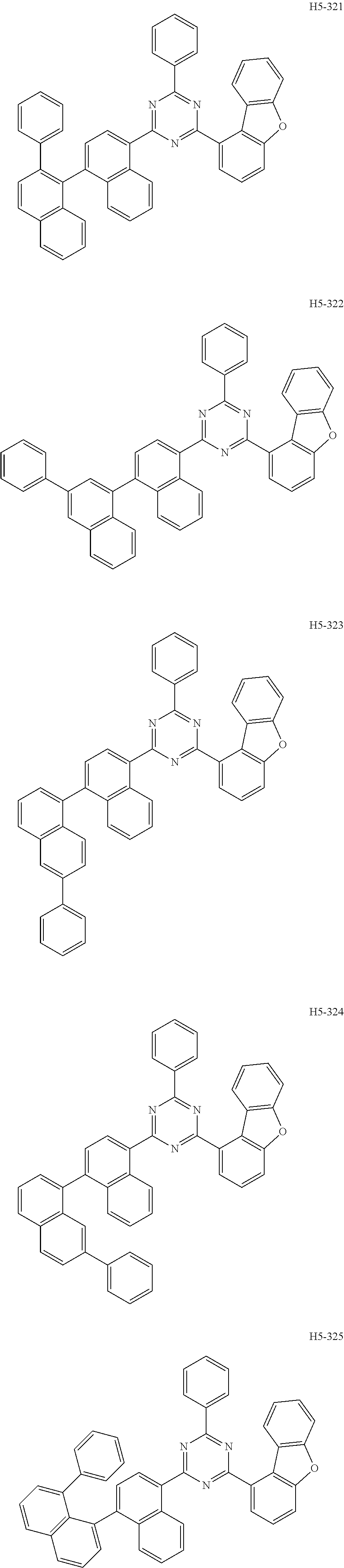 [SMILES: c1ccc(-c2nc(-c3ccc(-c4c(-c5ccccc5)ccc5ccccc45)c4ccccc34)nc(-c3cccc4oc5ccccc5c34)n2)cc1 ; c1ccc(-c2cc(-c3ccc(-c4nc(-c5ccccc5)nc(-c5cccc6oc7ccccc7c56)n4)c4ccccc34)c3ccccc3c2)cc1 ; c1ccc(-c2ccc3c(-c4ccc(-c5nc(-c6ccccc6)nc(-c6cccc7oc8ccccc8c67)n5)c5ccccc45)cccc3c2)cc1 ; c1ccc(-c2ccc3cccc(-c4ccc(-c5nc(-c6ccccc6)nc(-c6cccc7oc8ccccc8c67)n5)c5ccccc45)c3c2)cc1 ; c1ccc(-c2nc(-c3ccc(-c4cccc5cccc(-c6ccccc6)c45)c4ccccc34)nc(-c3cccc4oc5ccccc5c34)n2)cc1] 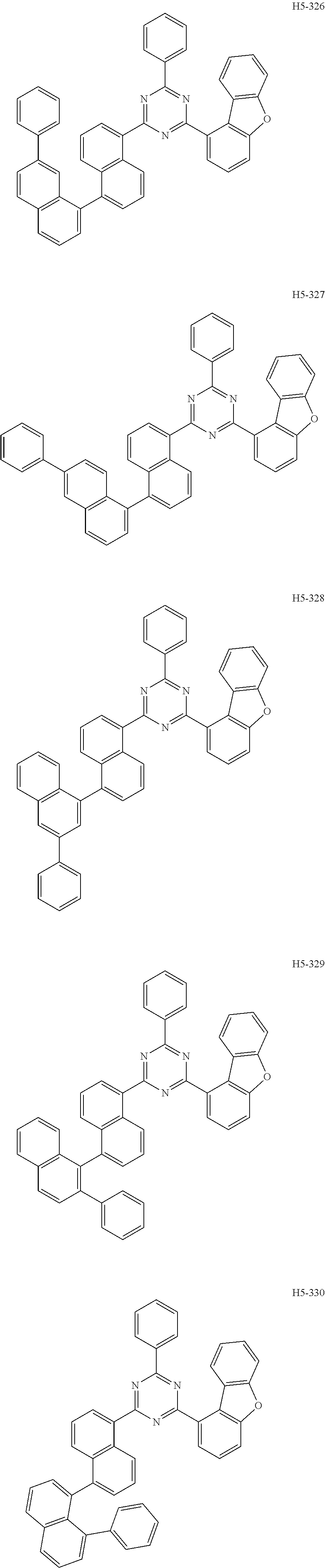 [SMILES: c1ccc(-c2ccc3cccc(-c4cccc5c(-c6nc(-c7ccccc7)nc(-c7cccc8oc9ccccc9c78)n6)cccc45)c3c2)cc1 ; c1ccc(-c2ccc3c(-c4cccc5c(-c6nc(-c7ccccc7)nc(-c7cccc8oc9ccccc9c78)n6)cccc45)cccc3c2)cc1 ; c1ccc(-c2cc(-c3cccc4c(-c5nc(-c6ccccc6)nc(-c6cccc7oc8ccccc8c67)n5)cccc34)c3ccccc3c2)cc1 ; c1ccc(-c2nc(-c3cccc4c(-c5c(-c6ccccc6)ccc6ccccc56)cccc34)nc(-c3cccc4oc5ccccc5c34)n2)cc1 ; c1ccc(-c2nc(-c3cccc4c(-c5cccc6cccc(-c7ccccc7)c56)cccc34)nc(-c3cccc4oc5ccccc5c34)n2)cc1] 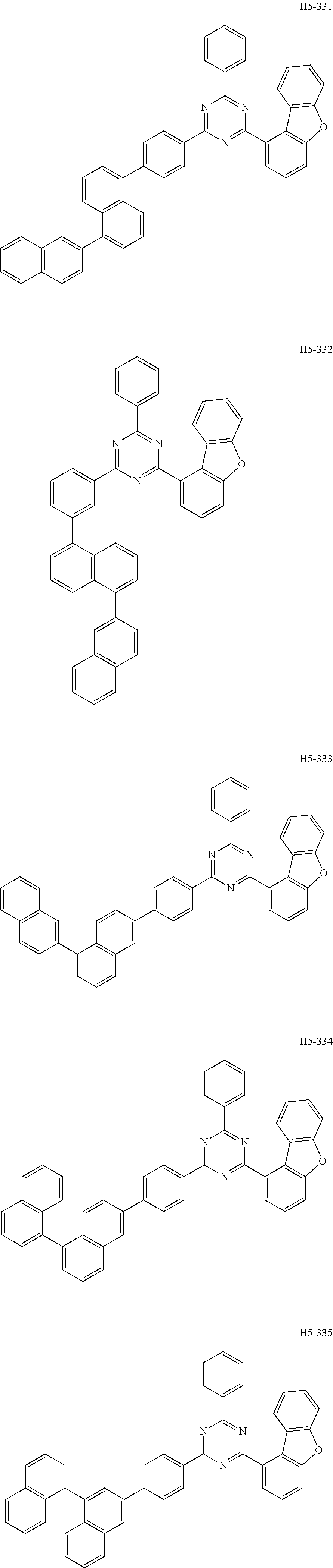 [SMILES: c1ccc(-c2nc(-c3ccc(-c4cccc5c(-c6ccc7ccccc7c6)cccc45)cc3)nc(-c3cccc4oc5ccccc5c34)n2)cc1 ; c1ccc(-c2nc(-c3cccc(-c4cccc5c(-c6ccc7ccccc7c6)cccc45)c3)nc(-c3cccc4oc5ccccc5c34)n2)cc1 ; c1ccc(-c2nc(-c3ccc(-c4ccc5c(-c6ccc7ccccc7c6)cccc5c4)cc3)nc(-c3cccc4oc5ccccc5c34)n2)cc1 ; c1ccc(-c2nc(-c3ccc(-c4ccc5c(-c6cccc7ccccc67)cccc5c4)cc3)nc(-c3cccc4oc5ccccc5c34)n2)cc1 ; c1ccc(-c2nc(-c3ccc(-c4cc(-c5cccc6ccccc56)c5ccccc5c4)cc3)nc(-c3cccc4oc5ccccc5c34)n2)cc1] 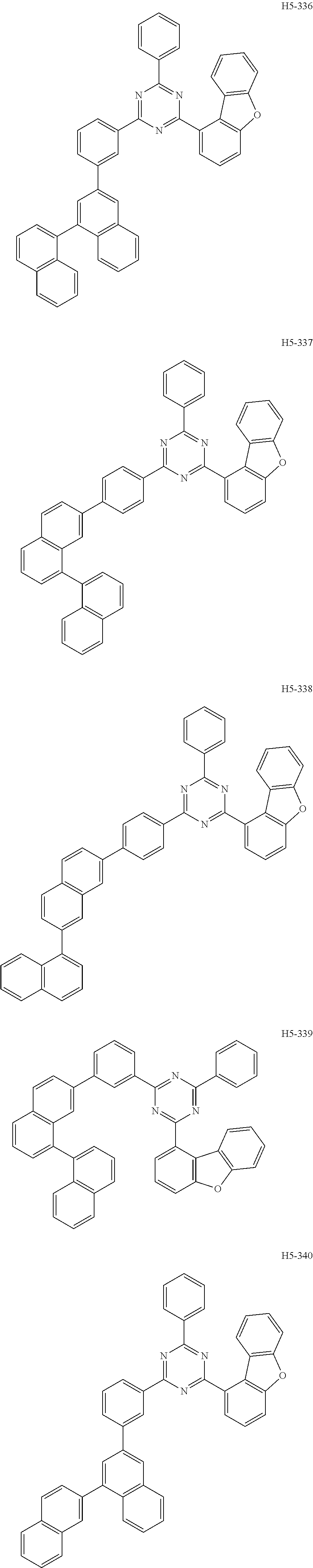 [SMILES: c1ccc(-c2nc(-c3cccc(-c4cc(-c5cccc6ccccc56)c5ccccc5c4)c3)nc(-c3cccc4oc5ccccc5c34)n2)cc1 ; c1ccc(-c2nc(-c3ccc(-c4ccc5cccc(-c6cccc7ccccc67)c5c4)cc3)nc(-c3cccc4oc5ccccc5c34)n2)cc1 ; c1ccc(-c2nc(-c3ccc(-c4ccc5ccc(-c6cccc7ccccc67)cc5c4)cc3)nc(-c3cccc4oc5ccccc5c34)n2)cc1 ; c1ccc(-c2nc(-c3cccc(-c4ccc5cccc(-c6cccc7ccccc67)c5c4)c3)nc(-c3cccc4oc5ccccc5c34)n2)cc1 ; c1ccc(-c2nc(-c3cccc(-c4cc(-c5ccc6ccccc6c5)c5ccccc5c4)c3)nc(-c3cccc4oc5ccccc5c34)n2)cc1] 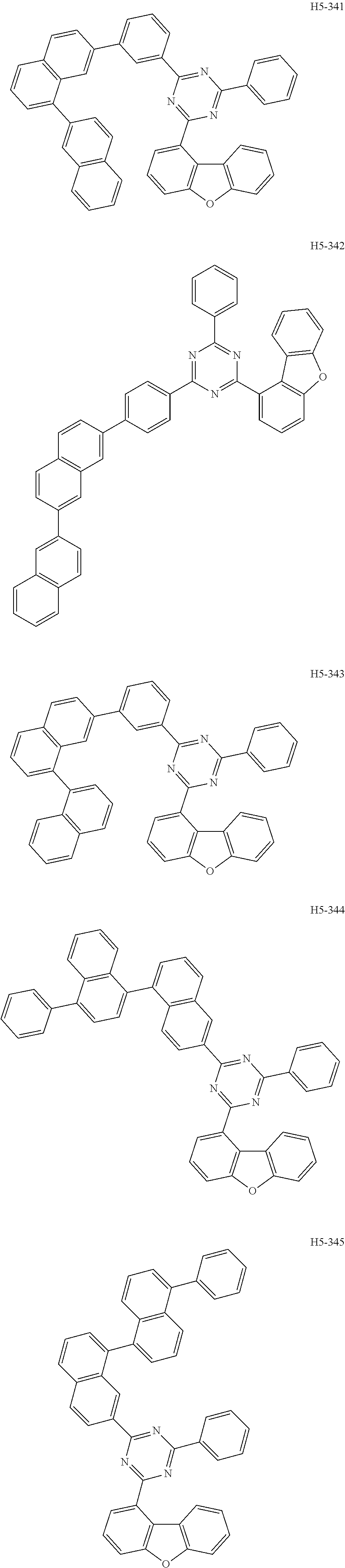 [SMILES: c1ccc(-c2nc(-c3cccc(-c4ccc5cccc(-c6ccc7ccccc7c6)c5c4)c3)nc(-c3cccc4oc5ccccc5c34)n2)cc1 ; c1ccc(-c2nc(-c3ccc(-c4ccc5ccc(-c6ccc7ccccc7c6)cc5c4)cc3)nc(-c3cccc4oc5ccccc5c34)n2)cc1 ; c1ccc(-c2nc(-c3cccc(-c4ccc5cccc(-c6cccc7ccccc67)c5c4)c3)nc(-c3cccc4oc5ccccc5c34)n2)cc1 ; c1ccc(-c2nc(-c3ccc4c(-c5ccc(-c6ccccc6)c6ccccc56)cccc4c3)nc(-c3cccc4oc5ccccc5c34)n2)cc1 ; c1ccc(-c2nc(-c3ccc4cccc(-c5cccc6c(-c7ccccc7)cccc56)c4c3)nc(-c3cccc4oc5ccccc5c34)n2)cc1] 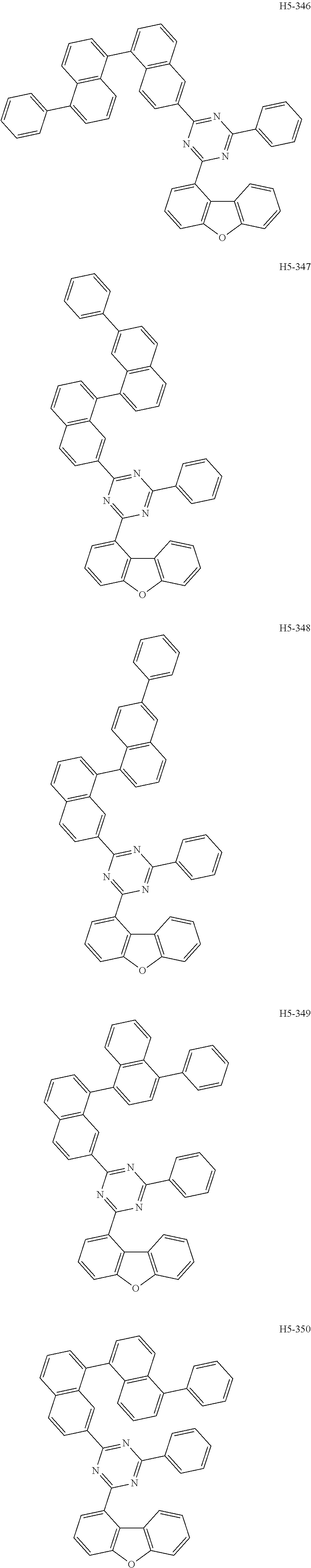 [SMILES: c1ccc(-c2nc(-c3ccc4c(-c5cccc6c(-c7ccccc7)cccc56)cccc4c3)nc(-c3cccc4oc5ccccc5c34)n2)cc1 ; c1ccc(-c2ccc3cccc(-c4cccc5ccc(-c6nc(-c7ccccc7)nc(-c7cccc8oc9ccccc9c78)n6)cc45)c3c2)cc1 ; c1ccc(-c2ccc3c(-c4cccc5ccc(-c6nc(-c7ccccc7)nc(-c7cccc8oc9ccccc9c78)n6)cc45)cccc3c2)cc1 ; c1ccc(-c2nc(-c3ccc4cccc(-c5ccc(-c6ccccc6)c6ccccc56)c4c3)nc(-c3cccc4oc5ccccc5c34)n2)cc1 ; c1ccc(-c2nc(-c3ccc4cccc(-c5cccc6c(-c7ccccc7)cccc56)c4c3)nc(-c3cccc4oc5ccccc5c34)n2)cc1] 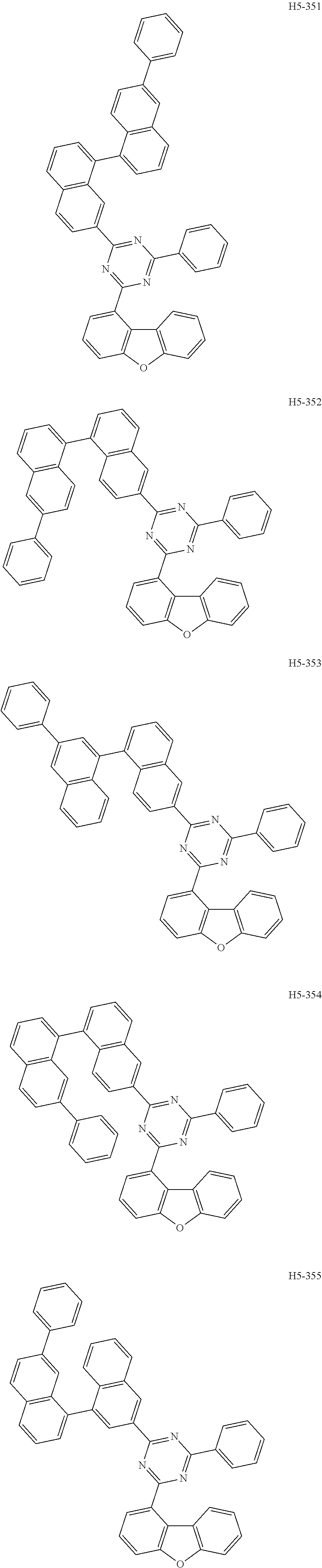 [SMILES: c1ccc(-c2ccc3c(-c4cccc5ccc(-c6nc(-c7ccccc7)nc(-c7cccc8oc9ccccc9c78)n6)cc45)cccc3c2)cc1 ; c1ccc(-c2ccc3c(-c4cccc5cc(-c6nc(-c7ccccc7)nc(-c7cccc8oc9ccccc9c78)n6)ccc45)cccc3c2)cc1 ; c1ccc(-c2cc(-c3cccc4cc(-c5nc(-c6ccccc6)nc(-c6cccc7oc8ccccc8c67)n5)ccc34)c3ccccc3c2)cc1 ; c1ccc(-c2ccc3cccc(-c4cccc5cc(-c6nc(-c7ccccc7)nc(-c7cccc8oc9ccccc9c78)n6)ccc45)c3c2)cc1 ; c1ccc(-c2ccc3cccc(-c4cc(-c5nc(-c6ccccc6)nc(-c6cccc7oc8ccccc8c67)n5)cc5ccccc45)c3c2)cc1] 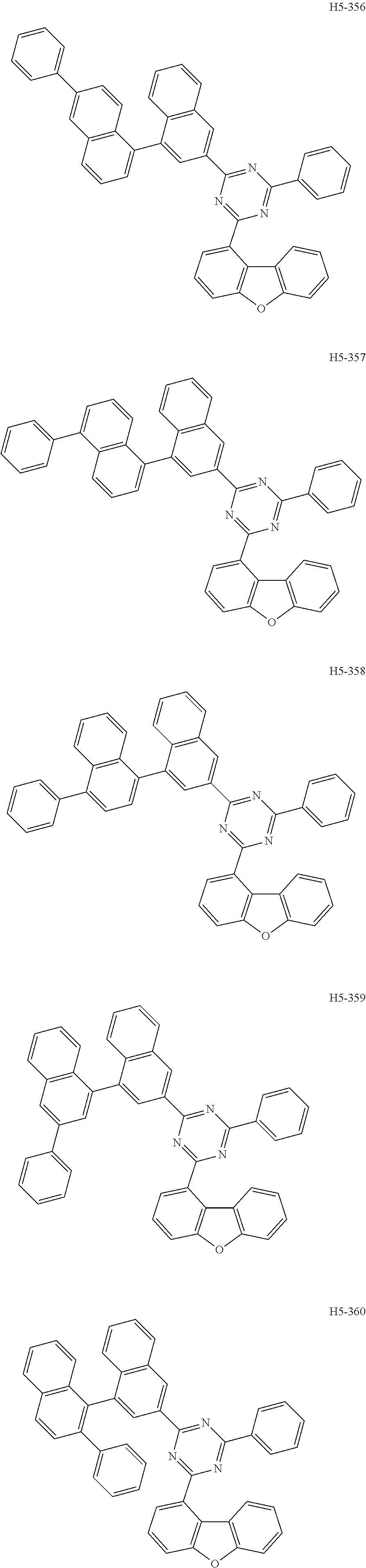 [SMILES: c1ccc(-c2ccc3c(-c4cc(-c5nc(-c6ccccc6)nc(-c6cccc7oc8ccccc8c67)n5)cc5ccccc45)cccc3c2)cc1 ; c1ccc(-c2nc(-c3cc(-c4cccc5c(-c6ccccc6)cccc45)c4ccccc4c3)nc(-c3cccc4oc5ccccc5c34)n2)cc1 ; c1ccc(-c2nc(-c3cc(-c4ccc(-c5ccccc5)c5ccccc45)c4ccccc4c3)nc(-c3cccc4oc5ccccc5c34)n2)cc1 ; c1ccc(-c2cc(-c3cc(-c4nc(-c5ccccc5)nc(-c5cccc6oc7ccccc7c56)n4)cc4ccccc34)c3ccccc3c2)cc1 ; c1ccc(-c2nc(-c3cc(-c4c(-c5ccccc5)ccc5ccccc45)c4ccccc4c3)nc(-c3cccc4oc5ccccc5c34)n2)cc1] 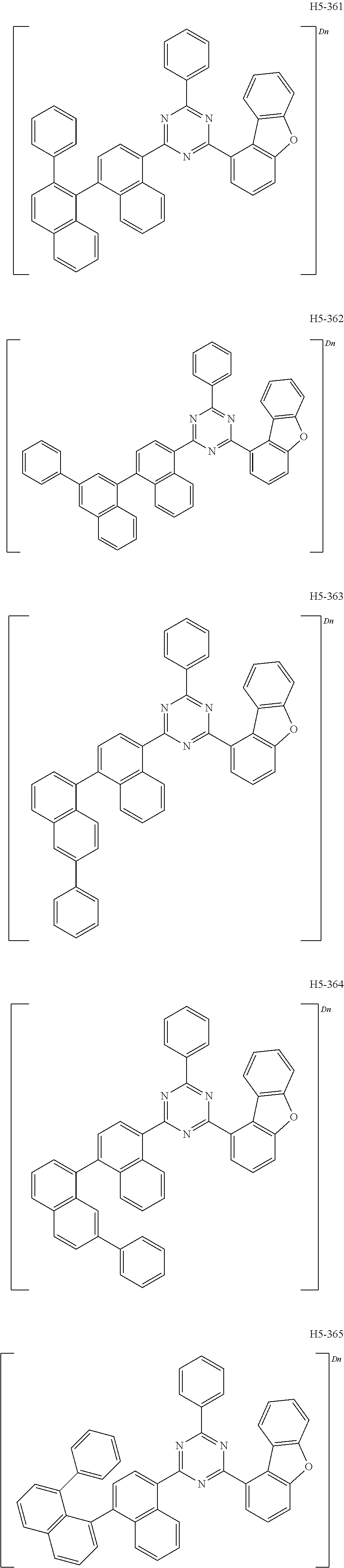 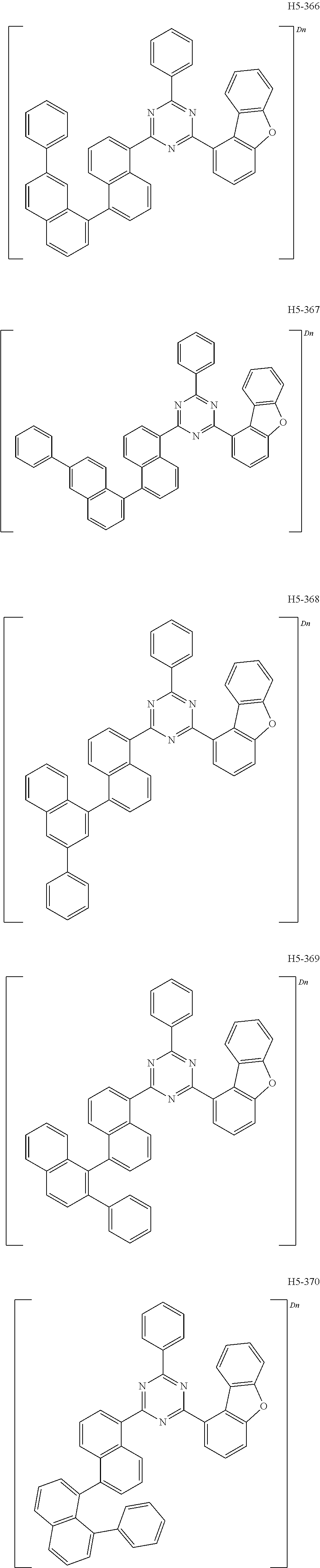 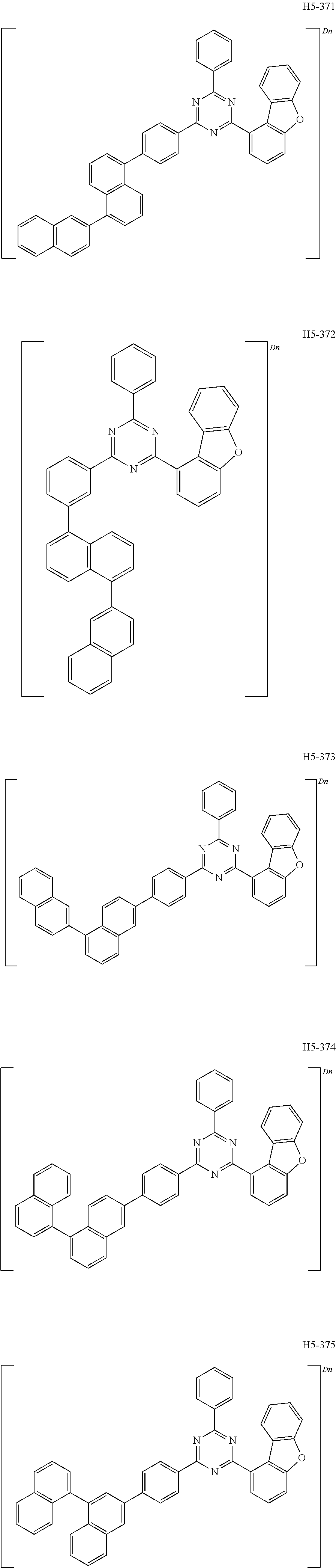 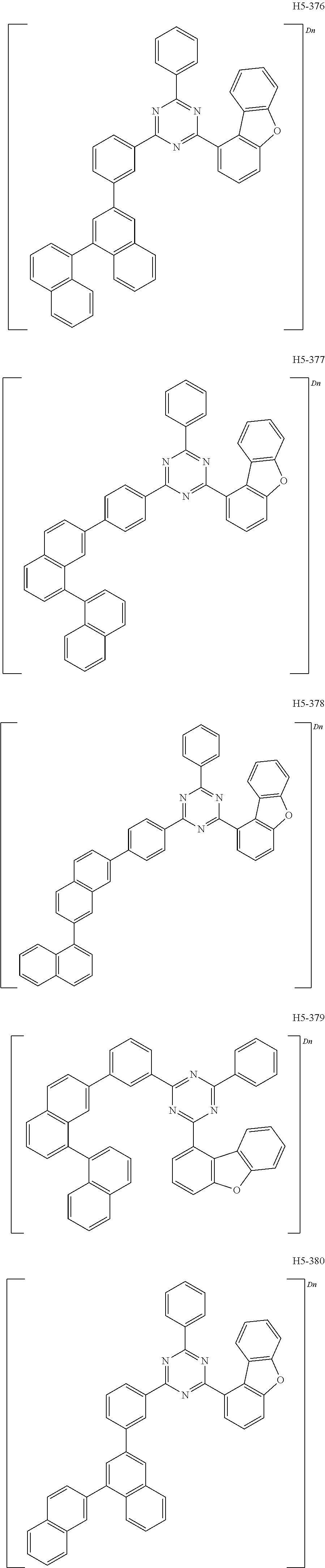 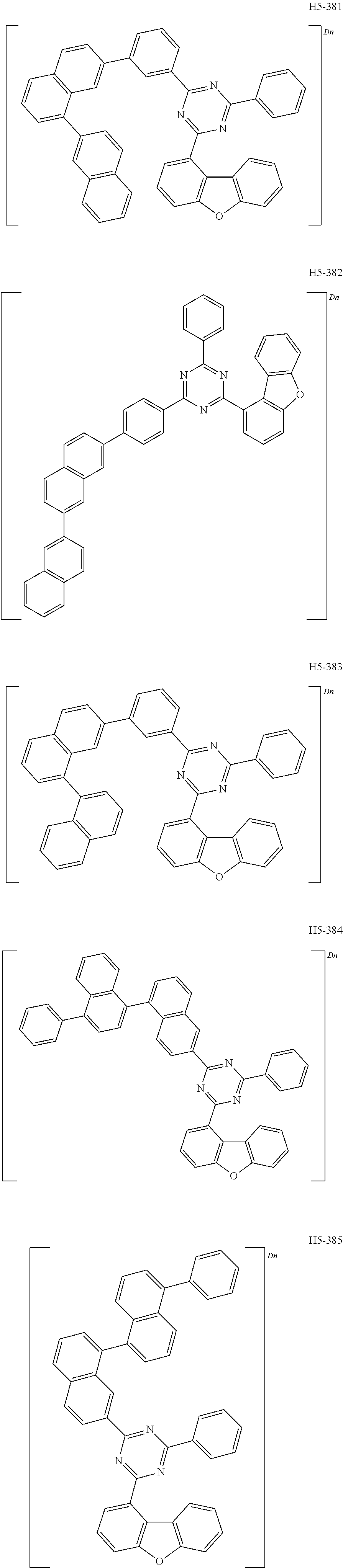 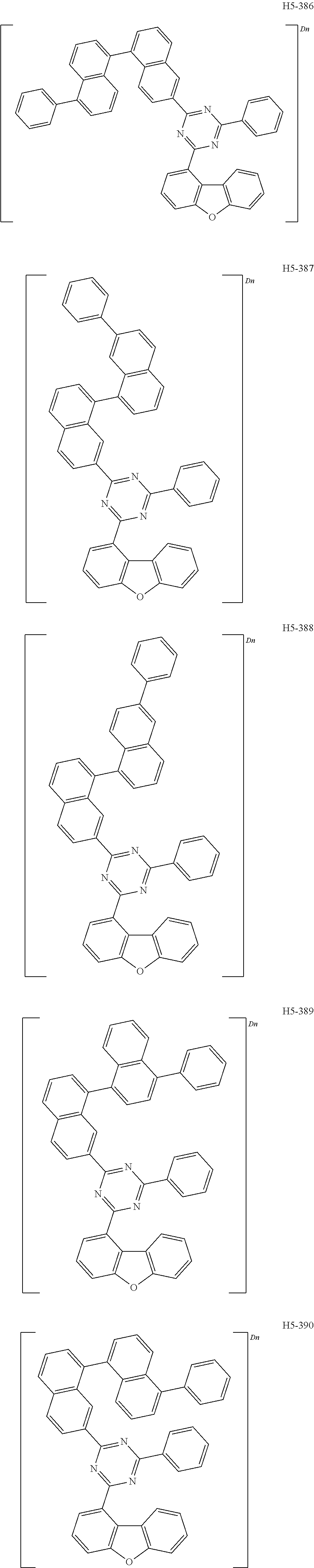 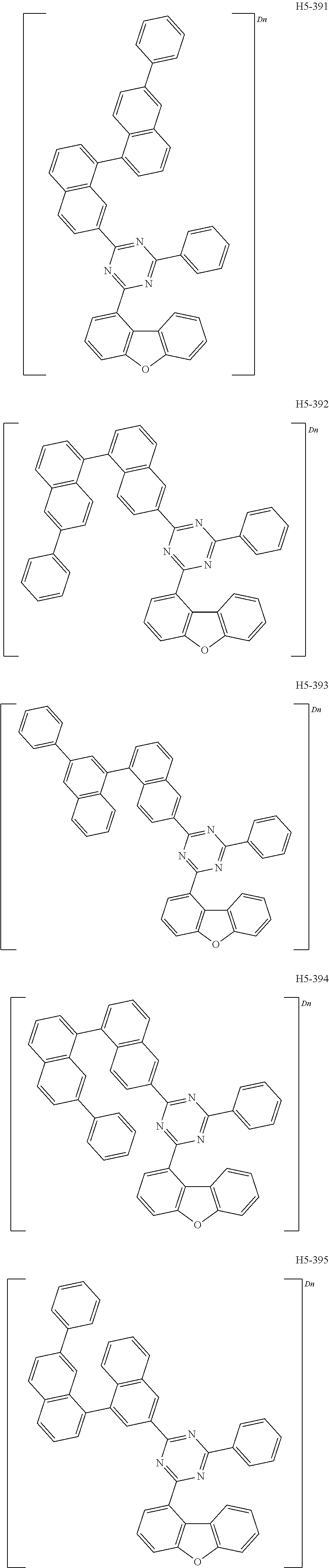 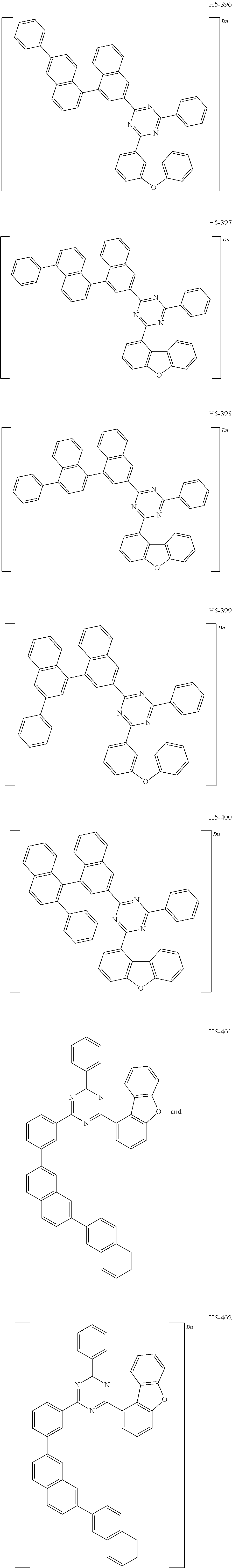
in the compounds above, Dₙ means that n number of hydrogens is replaced with deuterium, wherein n is an integer of 1 or more, and the upper limit of n is determined according to the number of hydrogens that may be substituted for each compound.

9. An organic electroluminescent compound represented by the following Formula 1-1: 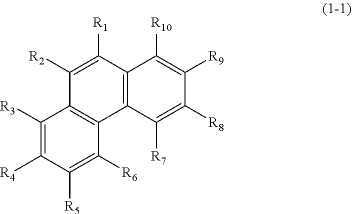
wherein
R₁ to R₁₀ each independently represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring of (C3-C30) aliphatic ring and (C6-C30)aromatic ring, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or *-L₁-N(Ar₁)(Ar₂); provided that at least one of R₁ to R₁₀ is *-L₁-N(Ar₁)(Ar₂);
L₁ represents a single bond, a substituted or unsubstituted (C6-C30)arylene, or a substituted or unsubstituted (3- to 30-membered)heteroarylene; and
Ar₁ and Ar₂ each independently represent a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl;
provided that if R₁, R₂, R₄, R₅, R₈, or R₉ is *-L₁-N(Ar₁)(Ar₂), the following conditions are satisfied:
(1) if R₅ or R₈ is *-L₁-N(Ar₁)(Ar₂), at least one of Ar₁ and Ar₂ is phenyl unsubstituted or substituted with deuterium;
(2) if R₁ or R₂ is *-L₁-N(Ar₁)(Ar₂), at least one of Ar₁ and Ar₂ is a substituted or unsubstituted dibenzofuranyl, or a substituted or unsubstituted carbazolyl; or all of Ar₁ and Ar₂ are a substituted or unsubstituted (3- to 30-membered)heteroaryl; and
(3) if R₄ or R₉ is *-L₁-N(Ar₁)(Ar₂), all of Ar₁ and Ar₂ are a substituted or unsubstituted (3-to 30-membered)heteroaryl; or at least one of Ar₁ and Ar₂ is a substituted or unsubstituted carbazolyl.

10. The organic electroluminescent compound according to claim 9, wherein the organic electroluminescent compound represented by Formula 1-1 is selected from the following compounds:

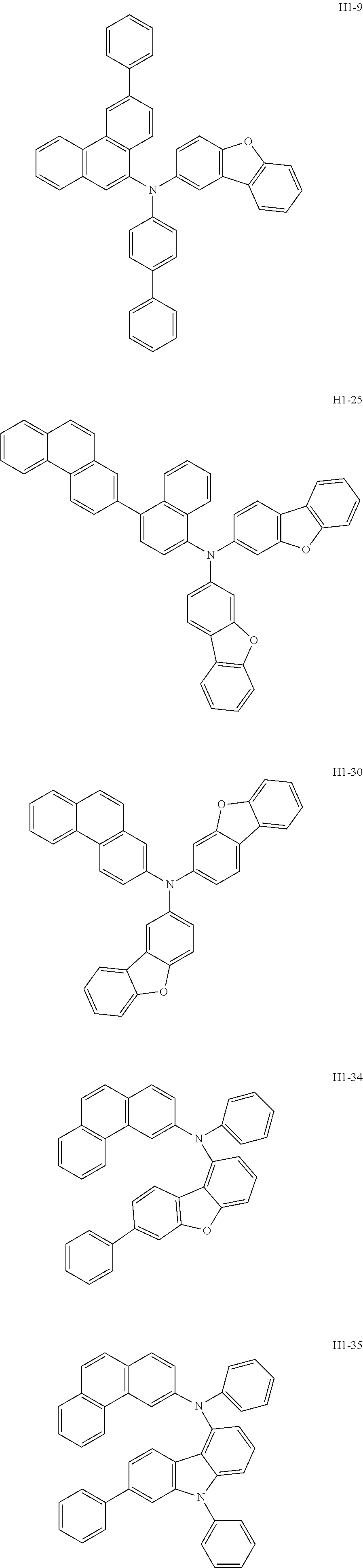 [SMILES: c1ccc(-c2ccc(N(c3ccc4oc5ccccc5c4c3)c3cc4ccccc4c4cc(-c5ccccc5)ccc34)cc2)cc1 ; c1ccc2c(c1)ccc1cc(-c3ccc(N(c4ccc5c(c4)oc4ccccc45)c4ccc5c(c4)oc4ccccc45)c4ccccc34)ccc12 ; c1ccc2c(c1)ccc1cc(N(c3ccc4c(c3)oc3ccccc34)c3ccc4oc5ccccc5c4c3)ccc12 ; c1ccc(-c2ccc3c(c2)oc2cccc(N(c4ccccc4)c4ccc5ccc6ccccc6c5c4)c23)cc1 ; c1ccc(-c2ccc3c4c(N(c5ccccc5)c5ccc6ccc7ccccc7c6c5)cccc4n(-c4ccccc4)c3c2)cc1]

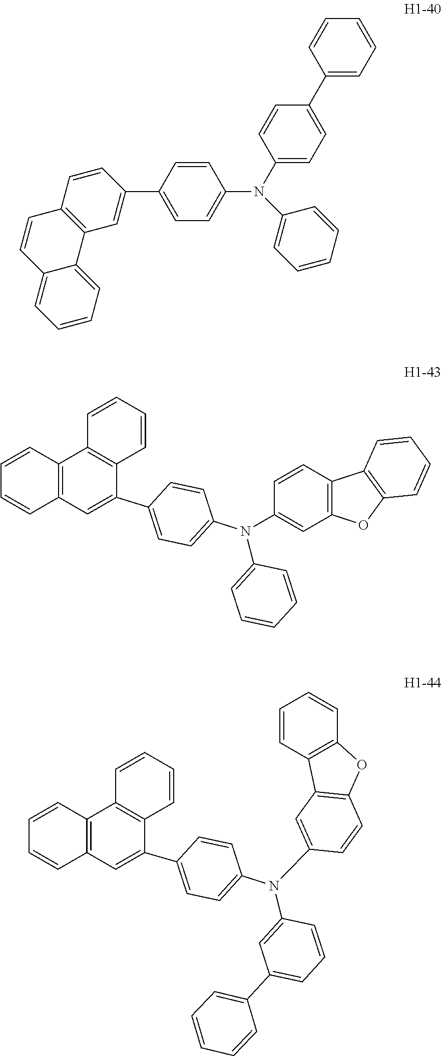 [SMILES: c1ccc(-c2ccc(N(c3ccccc3)c3ccc(-c4ccc5ccc6ccccc6c5c4)cc3)cc2)cc1 ; c1ccc(N(c2ccc(-c3cc4ccccc4c4ccccc34)cc2)c2ccc3c(c2)oc2ccccc23)cc1 ; c1ccc(-c2cccc(N(c3ccc(-c4cc5ccccc5c5ccccc45)cc3)c3ccc4oc5ccccc5c4c3)c2)cc1]

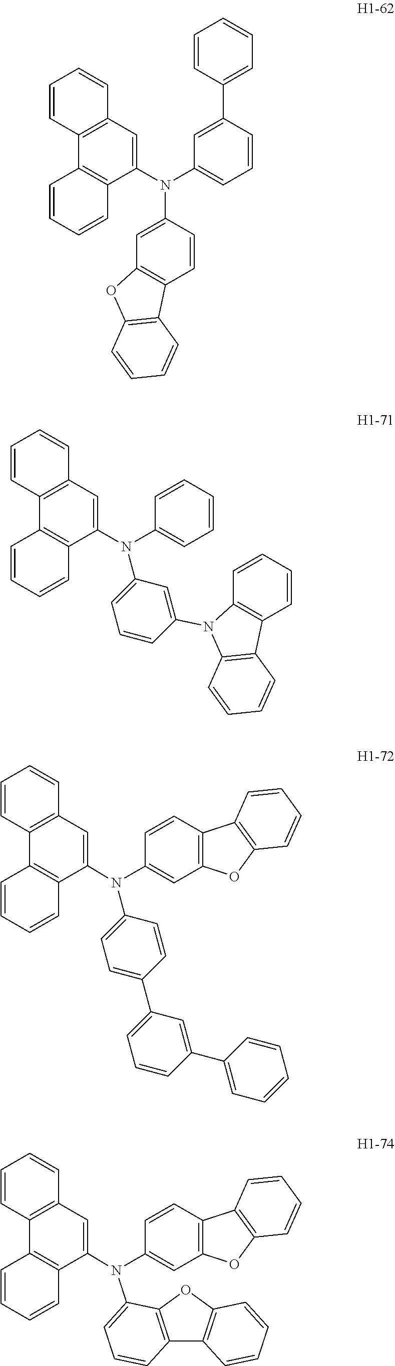 [SMILES: c1ccc(-c2cccc(N(c3ccc4c(c3)oc3ccccc34)c3cc4ccccc4c4ccccc34)c2)cc1 ; c1ccc(N(c2cccc(-n3c4ccccc4c4ccccc43)c2)c2cc3ccccc3c3ccccc23)cc1 ; c1ccc(-c2cccc(-c3ccc(N(c4ccc5c(c4)oc4ccccc45)c4cc5ccccc5c5ccccc45)cc3)c2)cc1 ; c1ccc2c(c1)cc(N(c1ccc3c(c1)oc1ccccc13)c1cccc3c1oc1ccccc13)c1ccccc12]

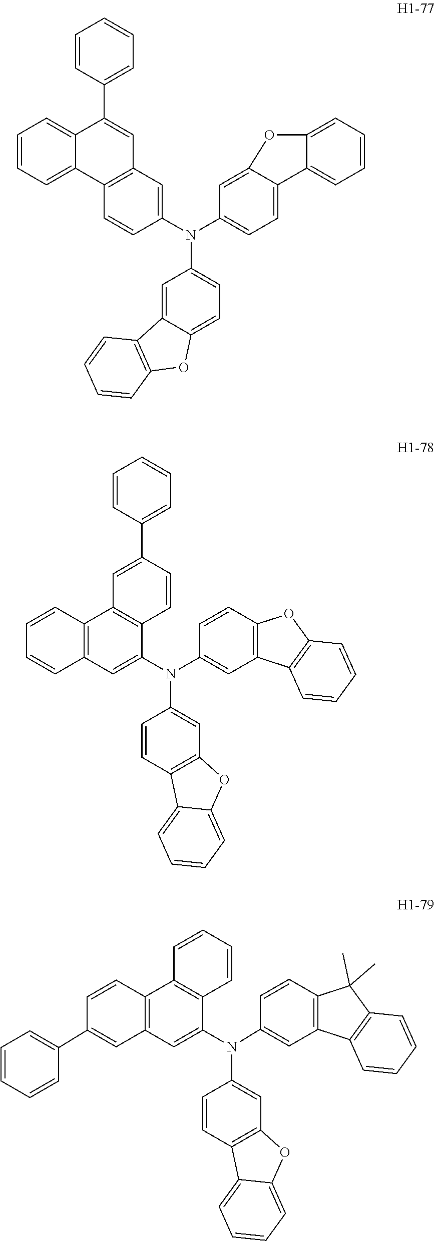 [SMILES: c1ccc(-c2cc3cc(N(c4ccc5c(c4)oc4ccccc45)c4ccc5oc6ccccc6c5c4)ccc3c3ccccc23)cc1 ; c1ccc(-c2ccc3c(N(c4ccc5c(c4)oc4ccccc45)c4ccc5oc6ccccc6c5c4)cc4ccccc4c3c2)cc1 ; CC1(C)c2ccccc2-c2cc(N(c3ccc4c(c3)oc3ccccc34)c3cc4cc(-c5ccccc5)ccc4c4ccccc34)ccc21]

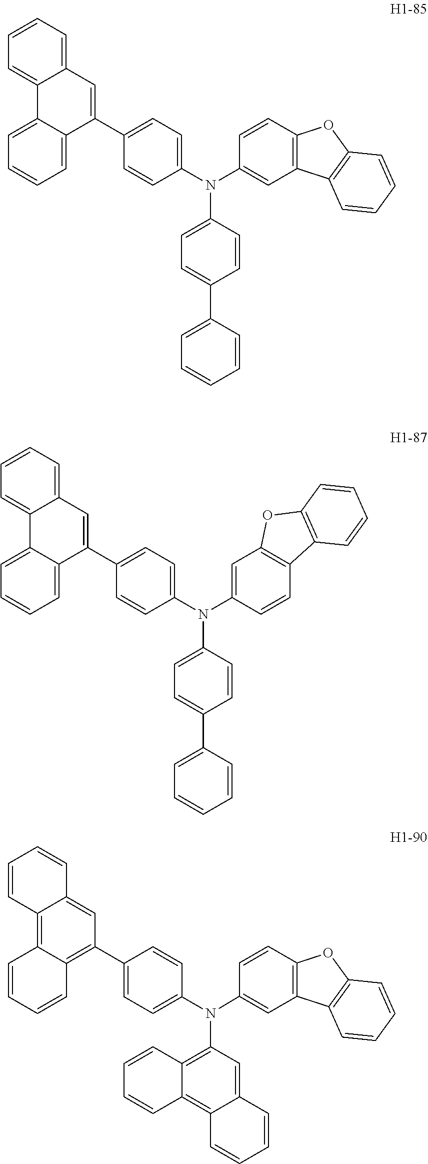 [SMILES: c1ccc(-c2ccc(N(c3ccc(-c4cc5ccccc5c5ccccc45)cc3)c3ccc4oc5ccccc5c4c3)cc2)cc1 ; c1ccc(-c2ccc(N(c3ccc(-c4cc5ccccc5c5ccccc45)cc3)c3ccc4c(c3)oc3ccccc34)cc2)cc1 ; c1ccc2c(c1)cc(-c1ccc(N(c3ccc4oc5ccccc5c4c3)c3cc4ccccc4c4ccccc34)cc1)c1ccccc12]

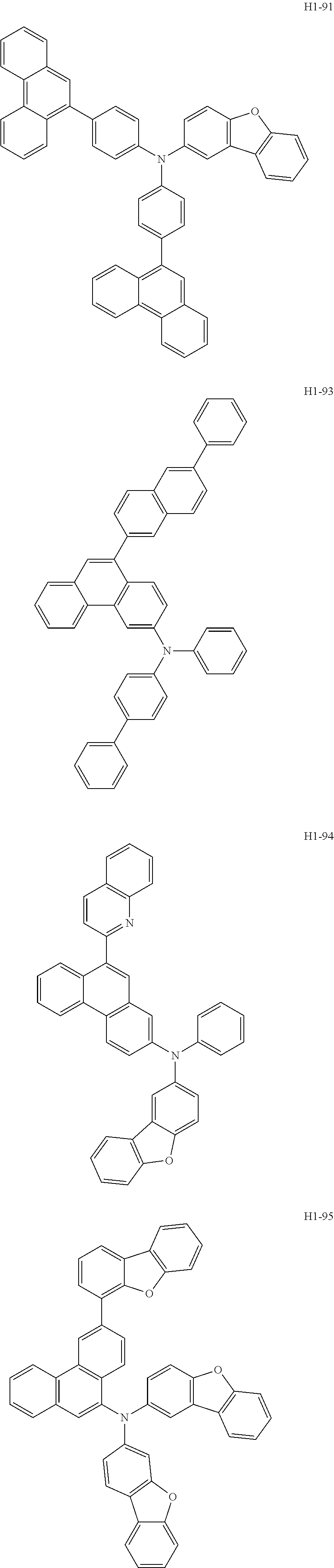 [SMILES: c1ccc2c(c1)cc(-c1ccc(N(c3ccc(-c4cc5ccccc5c5ccccc45)cc3)c3ccc4oc5ccccc5c4c3)cc1)c1ccccc12 ; c1ccc(-c2ccc(N(c3ccccc3)c3ccc4c(-c5ccc6cc(-c7ccccc7)ccc6c5)cc5ccccc5c4c3)cc2)cc1 ; c1ccc(N(c2ccc3c(c2)cc(-c2ccc4ccccc4n2)c2ccccc23)c2ccc3oc4ccccc4c3c2)cc1 ; c1ccc2c(c1)cc(N(c1ccc3c(c1)oc1ccccc13)c1ccc3oc4ccccc4c3c1)c1ccc(-c3cccc4c3oc3ccccc34)cc12]

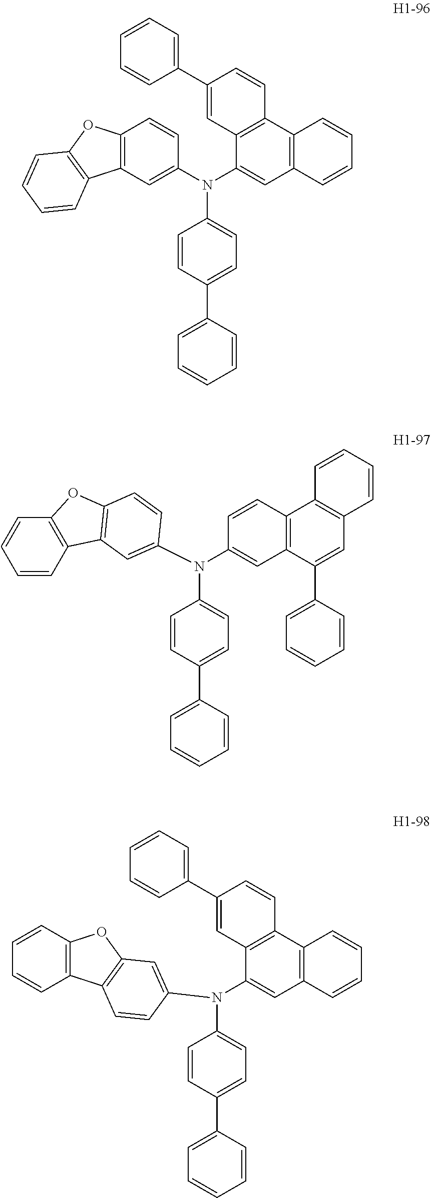 [SMILES: c1ccc(-c2ccc(N(c3ccc4oc5ccccc5c4c3)c3cc4ccccc4c4ccc(-c5ccccc5)cc34)cc2)cc1 ; c1ccc(-c2ccc(N(c3ccc4c(c3)c(-c3ccccc3)cc3ccccc34)c3ccc4oc5ccccc5c4c3)cc2)cc1 ; c1ccc(-c2ccc(N(c3ccc4c(c3)oc3ccccc34)c3cc4ccccc4c4ccc(-c5ccccc5)cc34)cc2)cc1]

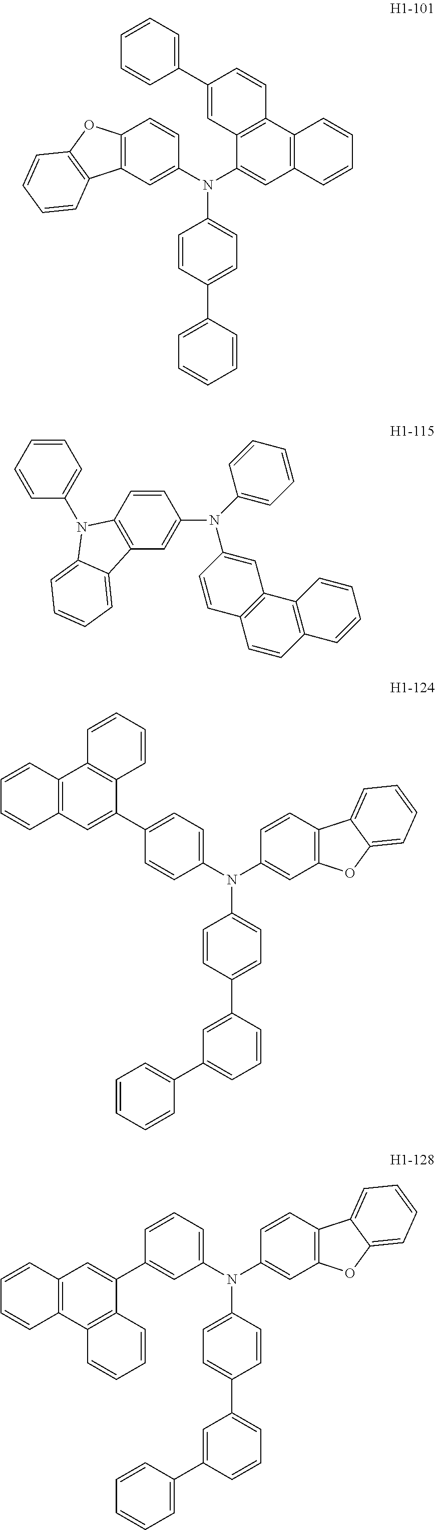 [SMILES: c1ccc(-c2ccc(N(c3ccc4oc5ccccc5c4c3)c3cc4ccccc4c4ccc(-c5ccccc5)cc34)cc2)cc1 ; c1ccc(N(c2ccc3ccc4ccccc4c3c2)c2ccc3c(c2)c2ccccc2n3-c2ccccc2)cc1 ; c1ccc(-c2cccc(-c3ccc(N(c4ccc(-c5cc6ccccc6c6ccccc56)cc4)c4ccc5c(c4)oc4ccccc45)cc3)c2)cc1 ; c1ccc(-c2cccc(-c3ccc(N(c4cccc(-c5cc6ccccc6c6ccccc56)c4)c4ccc5c(c4)oc4ccccc45)cc3)c2)cc1]

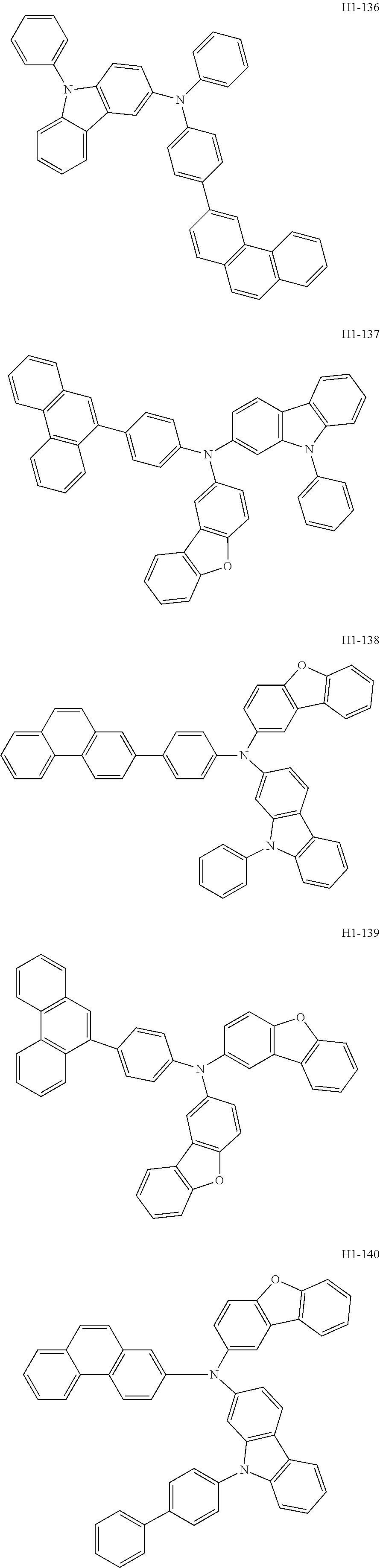 [SMILES: c1ccc(N(c2ccc(-c3ccc4ccc5ccccc5c4c3)cc2)c2ccc3c(c2)c2ccccc2n3-c2ccccc2)cc1 ; c1ccc(-n2c3ccccc3c3ccc(N(c4ccc(-c5cc6ccccc6c6ccccc56)cc4)c4ccc5oc6ccccc6c5c4)cc32)cc1 ; c1ccc(-n2c3ccccc3c3ccc(N(c4ccc(-c5ccc6c(ccc7ccccc76)c5)cc4)c4ccc5oc6ccccc6c5c4)cc32)cc1 ; c1ccc2c(c1)cc(-c1ccc(N(c3ccc4oc5ccccc5c4c3)c3ccc4oc5ccccc5c4c3)cc1)c1ccccc12 ; c1ccc(-c2ccc(-n3c4ccccc4c4ccc(N(c5ccc6c(ccc7ccccc76)c5)c5ccc6oc7ccccc7c6c5)cc43)cc2)cc1]

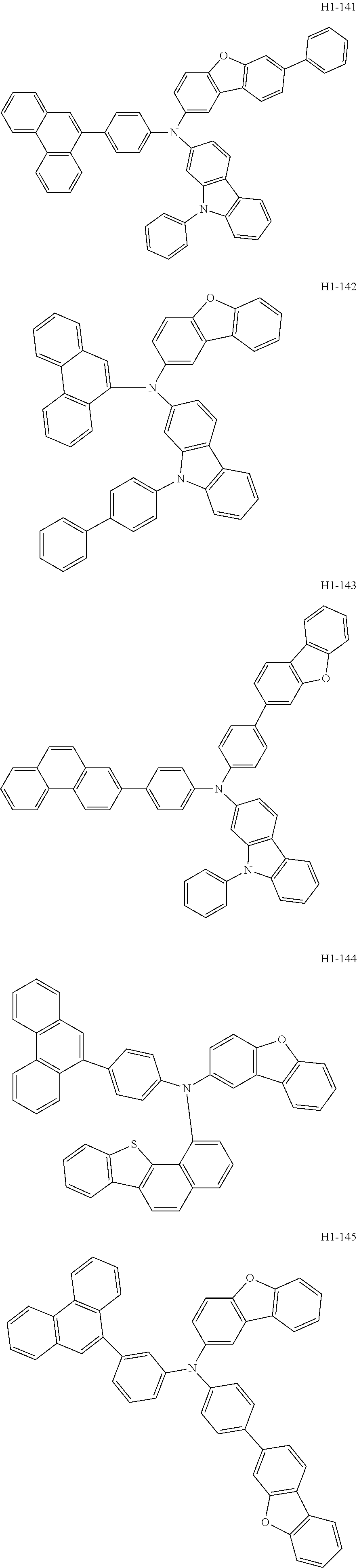 [SMILES: c1ccc(-c2ccc3c(c2)oc2ccc(N(c4ccc(-c5cc6ccccc6c6ccccc56)cc4)c4ccc5c6ccccc6n(-c6ccccc6)c5c4)cc23)cc1 ; c1ccc(-c2ccc(-n3c4ccccc4c4ccc(N(c5ccc6oc7ccccc7c6c5)c5cc6ccccc6c6ccccc56)cc43)cc2)cc1 ; c1ccc(-n2c3ccccc3c3ccc(N(c4ccc(-c5ccc6c(ccc7ccccc76)c5)cc4)c4ccc(-c5ccc6c(c5)oc5ccccc56)cc4)cc32)cc1 ; c1ccc2c(c1)cc(-c1ccc(N(c3ccc4oc5ccccc5c4c3)c3cccc4ccc5c6ccccc6sc5c34)cc1)c1ccccc12 ; c1cc(-c2cc3ccccc3c3ccccc23)cc(N(c2ccc(-c3ccc4c(c3)oc3ccccc34)cc2)c2ccc3oc4ccccc4c3c2)c1]

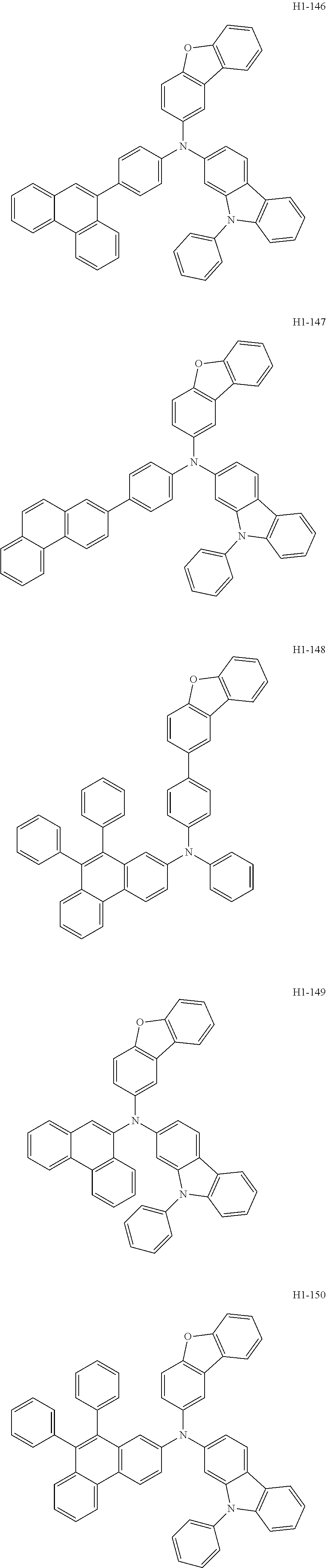 [SMILES: c1ccc(-n2c3ccccc3c3ccc(N(c4ccc(-c5cc6ccccc6c6ccccc56)cc4)c4ccc5oc6ccccc6c5c4)cc32)cc1 ; c1ccc(-n2c3ccccc3c3ccc(N(c4ccc(-c5ccc6c(ccc7ccccc76)c5)cc4)c4ccc5oc6ccccc6c5c4)cc32)cc1 ; c1ccc(-c2c(-c3ccccc3)c3cc(N(c4ccccc4)c4ccc(-c5ccc6oc7ccccc7c6c5)cc4)ccc3c3ccccc23)cc1 ; c1ccc(-n2c3ccccc3c3ccc(N(c4ccc5oc6ccccc6c5c4)c4cc5ccccc5c5ccccc45)cc32)cc1 ; c1ccc(-c2c(-c3ccccc3)c3cc(N(c4ccc5oc6ccccc6c5c4)c4ccc5c6ccccc6n(-c6ccccc6)c5c4)ccc3c3ccccc23)cc1]

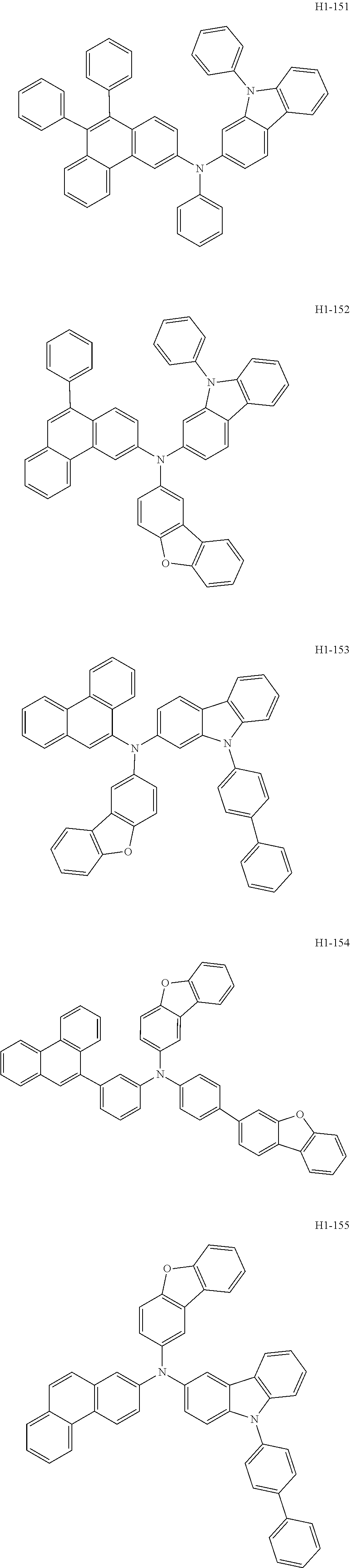 [SMILES: c1ccc(-c2c(-c3ccccc3)c3ccc(N(c4ccccc4)c4ccc5c6ccccc6n(-c6ccccc6)c5c4)cc3c3ccccc23)cc1 ; c1ccc(-c2cc3ccccc3c3cc(N(c4ccc5oc6ccccc6c5c4)c4ccc5c6ccccc6n(-c6ccccc6)c5c4)ccc23)cc1 ; c1ccc(-c2ccc(-n3c4ccccc4c4ccc(N(c5ccc6oc7ccccc7c6c5)c5cc6ccccc6c6ccccc56)cc43)cc2)cc1 ; c1cc(-c2cc3ccccc3c3ccccc23)cc(N(c2ccc(-c3ccc4c(c3)oc3ccccc34)cc2)c2ccc3oc4ccccc4c3c2)c1 ; c1ccc(-c2ccc(-n3c4ccccc4c4cc(N(c5ccc6c(ccc7ccccc76)c5)c5ccc6oc7ccccc7c6c5)ccc43)cc2)cc1]

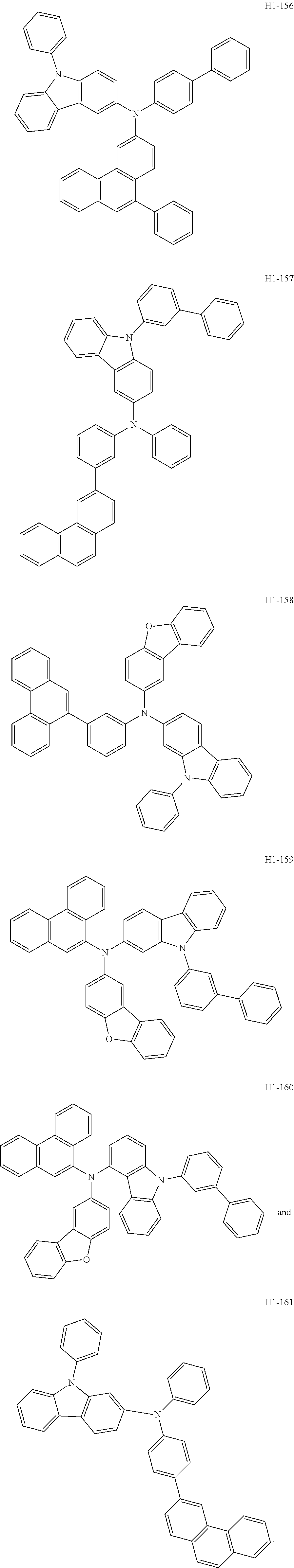

11. An organic electroluminescent device comprising an organic electroluminescent compound according to claim 9.

12. An organic electroluminescent compound represented by the following Formula 3′: 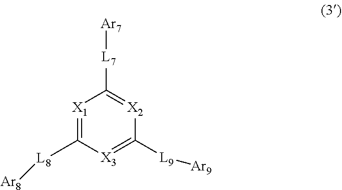
wherein
X₁ to X₃ each independently represent N or CR₁₁; provided that at least one of X₁ to X₃ is N;
R₁₁ represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring of (C3-C30) aliphatic ring and (C6-C30) aromatic ring;
L₇ to L₉ each independently represent a single bond, a substituted or unsubstituted (C6-C30)arylene, or a substituted or unsubstituted (3- to 30-membered)heteroarylene; and
Ar₇ to Ar₉ each independently represent a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring of (C3-C30) aliphatic ring and (C6-C30)aromatic ring, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₂₁-N(Ar₂₁)(Ar₂₂);
provided that at least one of Ar to Arg is Formula 2-2; 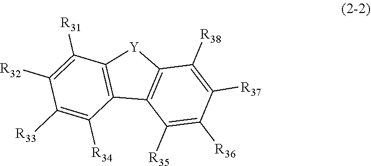
wherein,
Y represents —O— or —S—; and
R₃₁ to R₃₈ each independently represent a connection position connected to L₇ to L₉ in Formula 3; or hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to the adjacent substituents to form a ring(s);
provided that if one of L₇ to L₉ is a phenylene unsubstituted or substituted with deuterium or cyano, at least one of Ar₇ to Ar₉ is a binaphthyl unsubstituted or substituted with deuterium or cyano, and if one of L₇ to L₉ is a naphthylene unsubstituted or substituted with deuterium or cyano, at least one of Ar₇ to Ar₉ is a phenylnaphthyl unsubstituted or substituted with deuterium or cyano.

13. The organic electroluminescent compound according to claim 12, wherein the organic electroluminescent compound represented by Formula 3′ is selected from the following compounds: 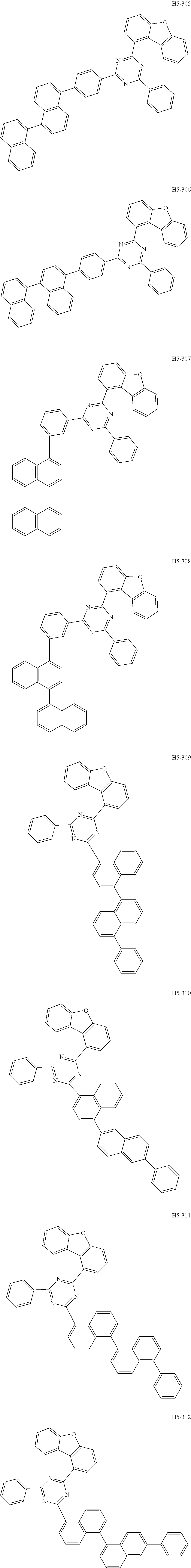 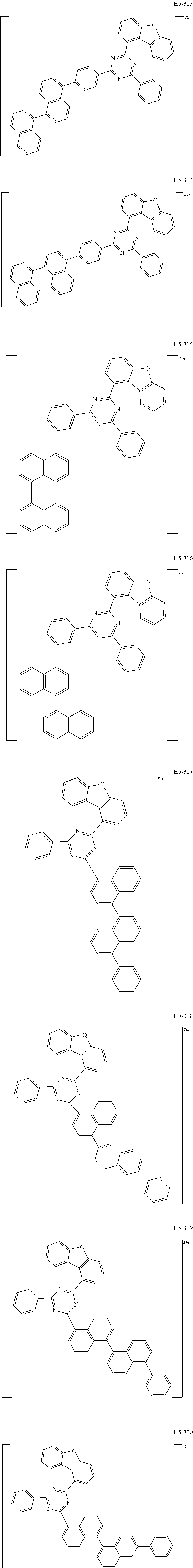 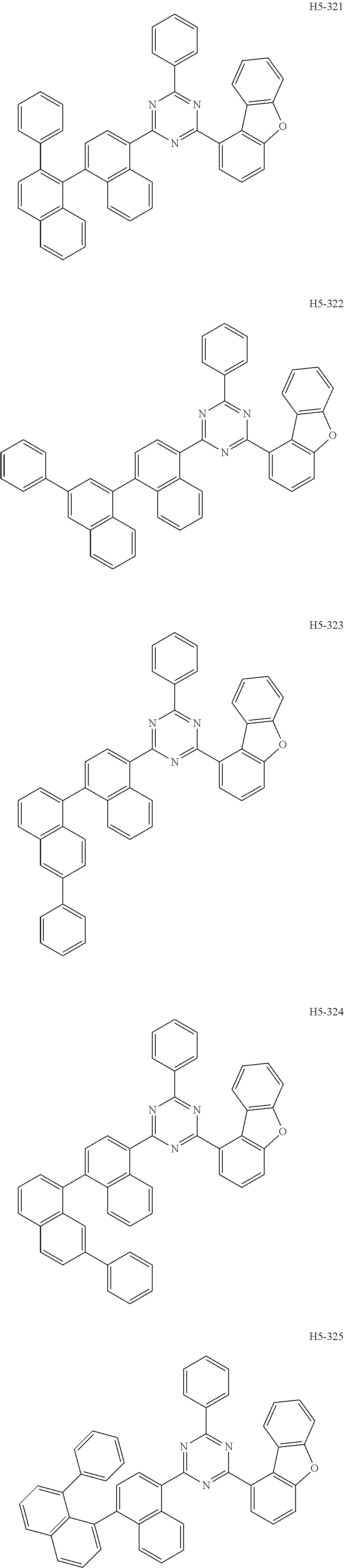 [SMILES: c1ccc(-c2nc(-c3ccc(-c4c(-c5ccccc5)ccc5ccccc45)c4ccccc34)nc(-c3cccc4oc5ccccc5c34)n2)cc1 ; c1ccc(-c2cc(-c3ccc(-c4nc(-c5ccccc5)nc(-c5cccc6oc7ccccc7c56)n4)c4ccccc34)c3ccccc3c2)cc1 ; c1ccc(-c2ccc3c(-c4ccc(-c5nc(-c6ccccc6)nc(-c6cccc7oc8ccccc8c67)n5)c5ccccc45)cccc3c2)cc1 ; c1ccc(-c2ccc3cccc(-c4ccc(-c5nc(-c6ccccc6)nc(-c6cccc7oc8ccccc8c67)n5)c5ccccc45)c3c2)cc1 ; c1ccc(-c2nc(-c3ccc(-c4cccc5cccc(-c6ccccc6)c45)c4ccccc34)nc(-c3cccc4oc5ccccc5c34)n2)cc1] 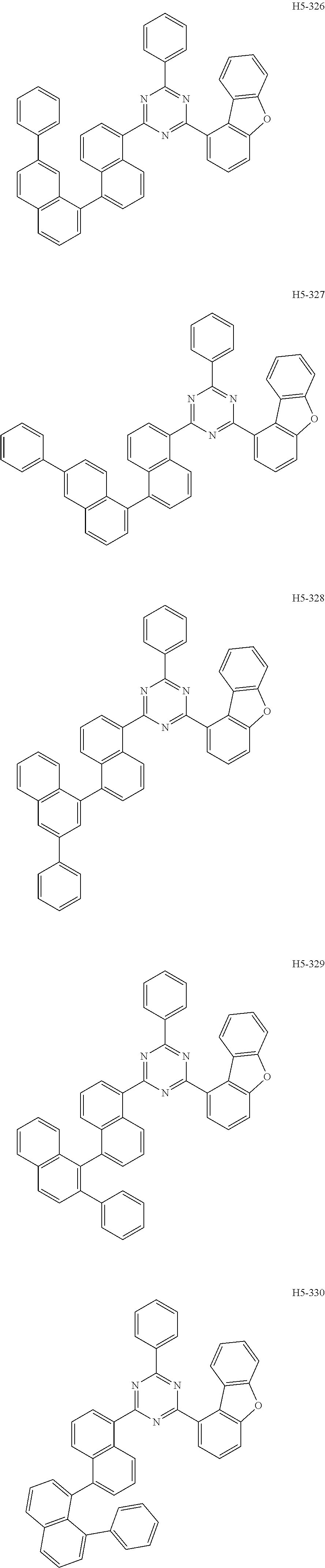 [SMILES: c1ccc(-c2ccc3cccc(-c4cccc5c(-c6nc(-c7ccccc7)nc(-c7cccc8oc9ccccc9c78)n6)cccc45)c3c2)cc1 ; c1ccc(-c2ccc3c(-c4cccc5c(-c6nc(-c7ccccc7)nc(-c7cccc8oc9ccccc9c78)n6)cccc45)cccc3c2)cc1 ; c1ccc(-c2cc(-c3cccc4c(-c5nc(-c6ccccc6)nc(-c6cccc7oc8ccccc8c67)n5)cccc34)c3ccccc3c2)cc1 ; c1ccc(-c2nc(-c3cccc4c(-c5c(-c6ccccc6)ccc6ccccc56)cccc34)nc(-c3cccc4oc5ccccc5c34)n2)cc1 ; c1ccc(-c2nc(-c3cccc4c(-c5cccc6cccc(-c7ccccc7)c56)cccc34)nc(-c3cccc4oc5ccccc5c34)n2)cc1] 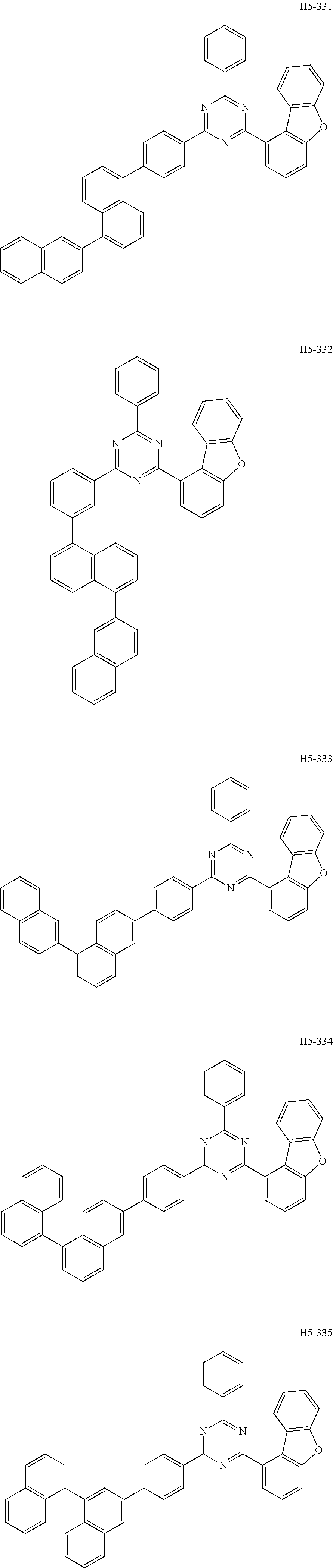 [SMILES: c1ccc(-c2nc(-c3ccc(-c4cccc5c(-c6ccc7ccccc7c6)cccc45)cc3)nc(-c3cccc4oc5ccccc5c34)n2)cc1 ; c1ccc(-c2nc(-c3cccc(-c4cccc5c(-c6ccc7ccccc7c6)cccc45)c3)nc(-c3cccc4oc5ccccc5c34)n2)cc1 ; c1ccc(-c2nc(-c3ccc(-c4ccc5c(-c6ccc7ccccc7c6)cccc5c4)cc3)nc(-c3cccc4oc5ccccc5c34)n2)cc1 ; c1ccc(-c2nc(-c3ccc(-c4ccc5c(-c6cccc7ccccc67)cccc5c4)cc3)nc(-c3cccc4oc5ccccc5c34)n2)cc1 ; c1ccc(-c2nc(-c3ccc(-c4cc(-c5cccc6ccccc56)c5ccccc5c4)cc3)nc(-c3cccc4oc5ccccc5c34)n2)cc1] 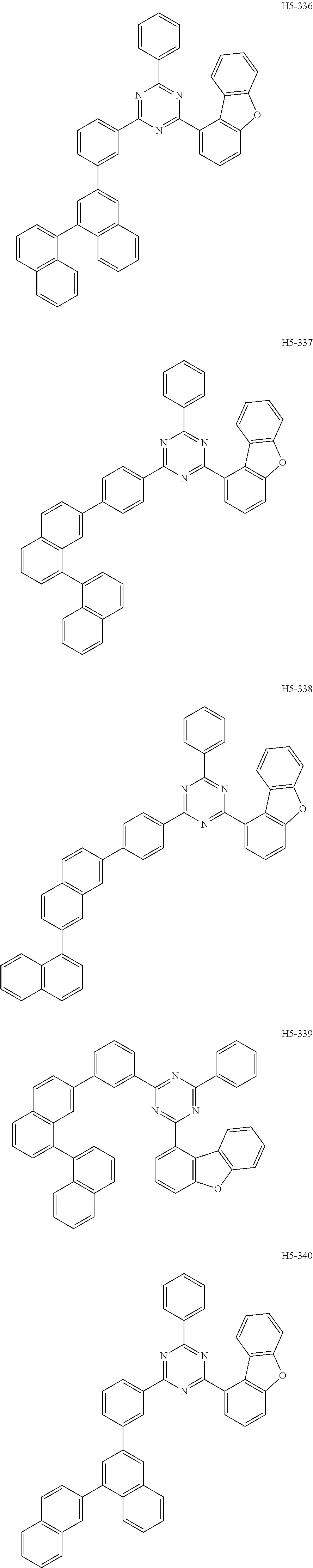 [SMILES: c1ccc(-c2nc(-c3cccc(-c4cc(-c5cccc6ccccc56)c5ccccc5c4)c3)nc(-c3cccc4oc5ccccc5c34)n2)cc1 ; c1ccc(-c2nc(-c3ccc(-c4ccc5cccc(-c6cccc7ccccc67)c5c4)cc3)nc(-c3cccc4oc5ccccc5c34)n2)cc1 ; c1ccc(-c2nc(-c3ccc(-c4ccc5ccc(-c6cccc7ccccc67)cc5c4)cc3)nc(-c3cccc4oc5ccccc5c34)n2)cc1 ; c1ccc(-c2nc(-c3cccc(-c4ccc5cccc(-c6cccc7ccccc67)c5c4)c3)nc(-c3cccc4oc5ccccc5c34)n2)cc1 ; c1ccc(-c2nc(-c3cccc(-c4cc(-c5ccc6ccccc6c5)c5ccccc5c4)c3)nc(-c3cccc4oc5ccccc5c34)n2)cc1] 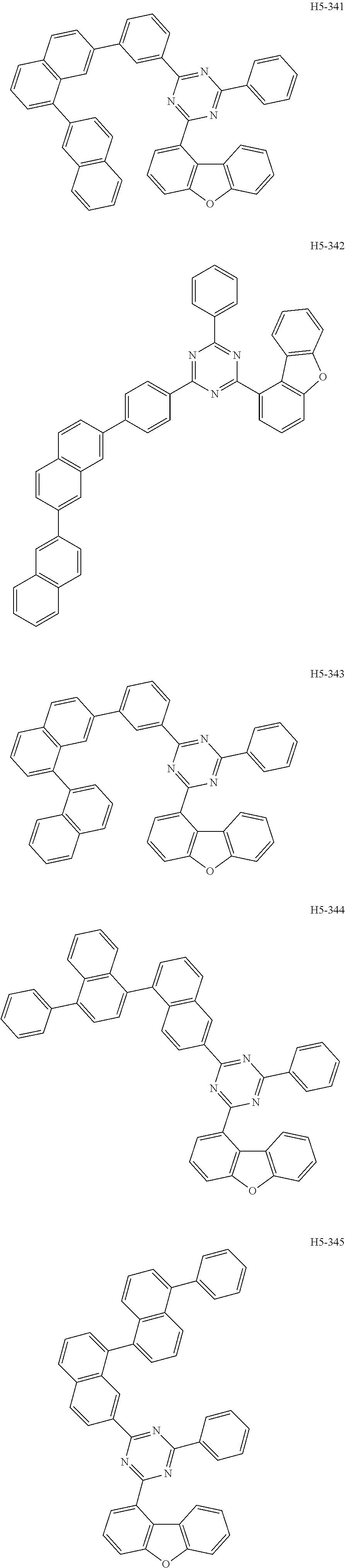 [SMILES: c1ccc(-c2nc(-c3cccc(-c4ccc5cccc(-c6ccc7ccccc7c6)c5c4)c3)nc(-c3cccc4oc5ccccc5c34)n2)cc1 ; c1ccc(-c2nc(-c3ccc(-c4ccc5ccc(-c6ccc7ccccc7c6)cc5c4)cc3)nc(-c3cccc4oc5ccccc5c34)n2)cc1 ; c1ccc(-c2nc(-c3cccc(-c4ccc5cccc(-c6cccc7ccccc67)c5c4)c3)nc(-c3cccc4oc5ccccc5c34)n2)cc1 ; c1ccc(-c2nc(-c3ccc4c(-c5ccc(-c6ccccc6)c6ccccc56)cccc4c3)nc(-c3cccc4oc5ccccc5c34)n2)cc1 ; c1ccc(-c2nc(-c3ccc4cccc(-c5cccc6c(-c7ccccc7)cccc56)c4c3)nc(-c3cccc4oc5ccccc5c34)n2)cc1] 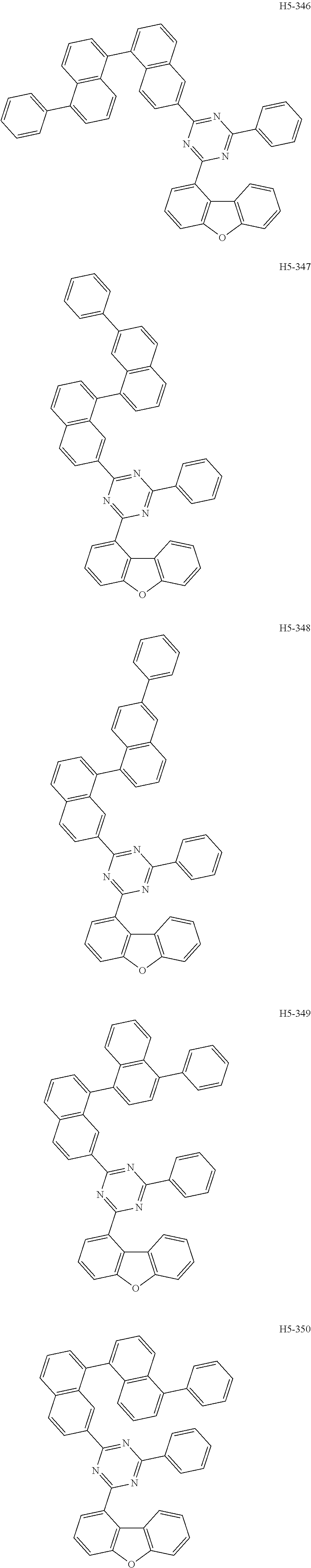 [SMILES: c1ccc(-c2nc(-c3ccc4c(-c5cccc6c(-c7ccccc7)cccc56)cccc4c3)nc(-c3cccc4oc5ccccc5c34)n2)cc1 ; c1ccc(-c2ccc3cccc(-c4cccc5ccc(-c6nc(-c7ccccc7)nc(-c7cccc8oc9ccccc9c78)n6)cc45)c3c2)cc1 ; c1ccc(-c2ccc3c(-c4cccc5ccc(-c6nc(-c7ccccc7)nc(-c7cccc8oc9ccccc9c78)n6)cc45)cccc3c2)cc1 ; c1ccc(-c2nc(-c3ccc4cccc(-c5ccc(-c6ccccc6)c6ccccc56)c4c3)nc(-c3cccc4oc5ccccc5c34)n2)cc1 ; c1ccc(-c2nc(-c3ccc4cccc(-c5cccc6c(-c7ccccc7)cccc56)c4c3)nc(-c3cccc4oc5ccccc5c34)n2)cc1] 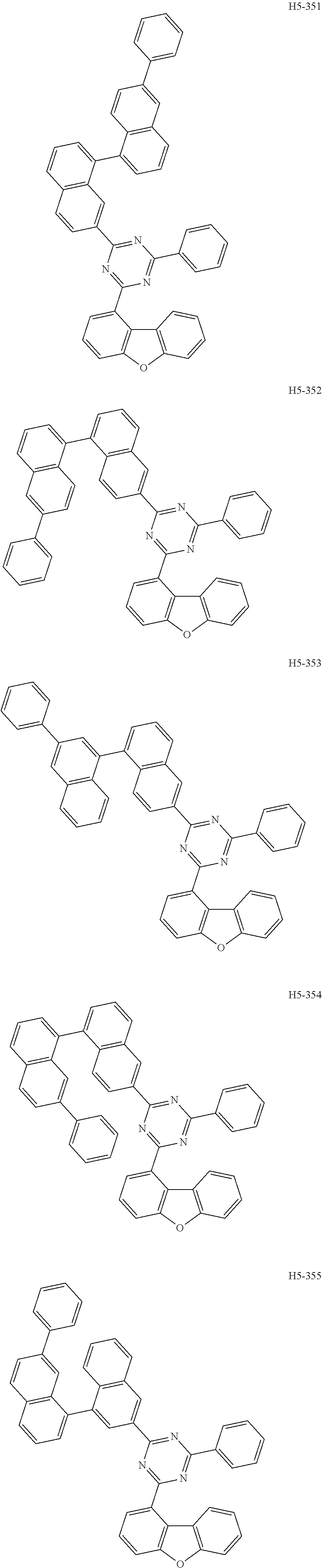 [SMILES: c1ccc(-c2ccc3c(-c4cccc5ccc(-c6nc(-c7ccccc7)nc(-c7cccc8oc9ccccc9c78)n6)cc45)cccc3c2)cc1 ; c1ccc(-c2ccc3c(-c4cccc5cc(-c6nc(-c7ccccc7)nc(-c7cccc8oc9ccccc9c78)n6)ccc45)cccc3c2)cc1 ; c1ccc(-c2cc(-c3cccc4cc(-c5nc(-c6ccccc6)nc(-c6cccc7oc8ccccc8c67)n5)ccc34)c3ccccc3c2)cc1 ; c1ccc(-c2ccc3cccc(-c4cccc5cc(-c6nc(-c7ccccc7)nc(-c7cccc8oc9ccccc9c78)n6)ccc45)c3c2)cc1 ; c1ccc(-c2ccc3cccc(-c4cc(-c5nc(-c6ccccc6)nc(-c6cccc7oc8ccccc8c67)n5)cc5ccccc45)c3c2)cc1] 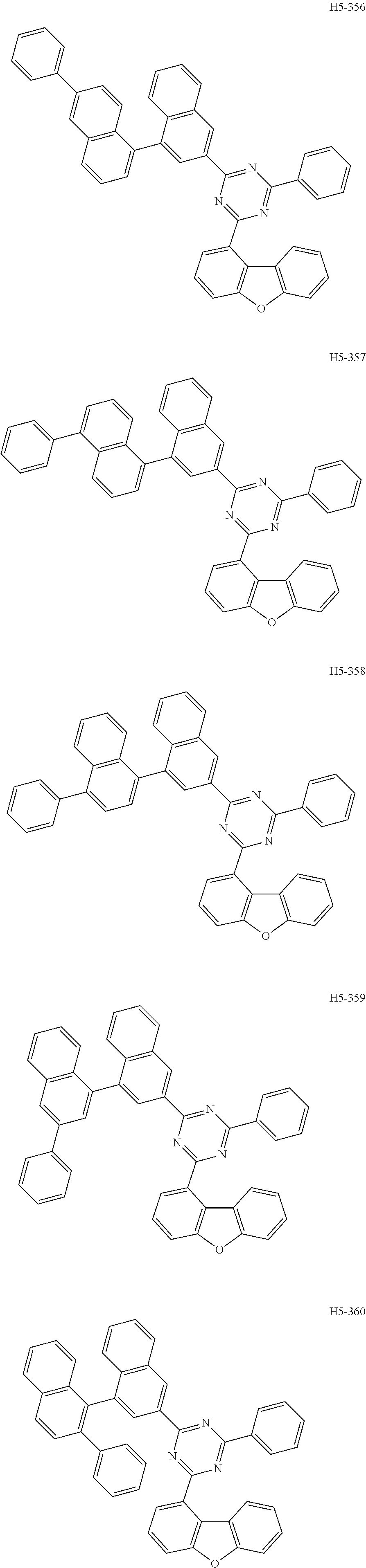 [SMILES: c1ccc(-c2ccc3c(-c4cc(-c5nc(-c6ccccc6)nc(-c6cccc7oc8ccccc8c67)n5)cc5ccccc45)cccc3c2)cc1 ; c1ccc(-c2nc(-c3cc(-c4cccc5c(-c6ccccc6)cccc45)c4ccccc4c3)nc(-c3cccc4oc5ccccc5c34)n2)cc1 ; c1ccc(-c2nc(-c3cc(-c4ccc(-c5ccccc5)c5ccccc45)c4ccccc4c3)nc(-c3cccc4oc5ccccc5c34)n2)cc1 ; c1ccc(-c2cc(-c3cc(-c4nc(-c5ccccc5)nc(-c5cccc6oc7ccccc7c56)n4)cc4ccccc34)c3ccccc3c2)cc1 ; c1ccc(-c2nc(-c3cc(-c4c(-c5ccccc5)ccc5ccccc45)c4ccccc4c3)nc(-c3cccc4oc5ccccc5c34)n2)cc1] 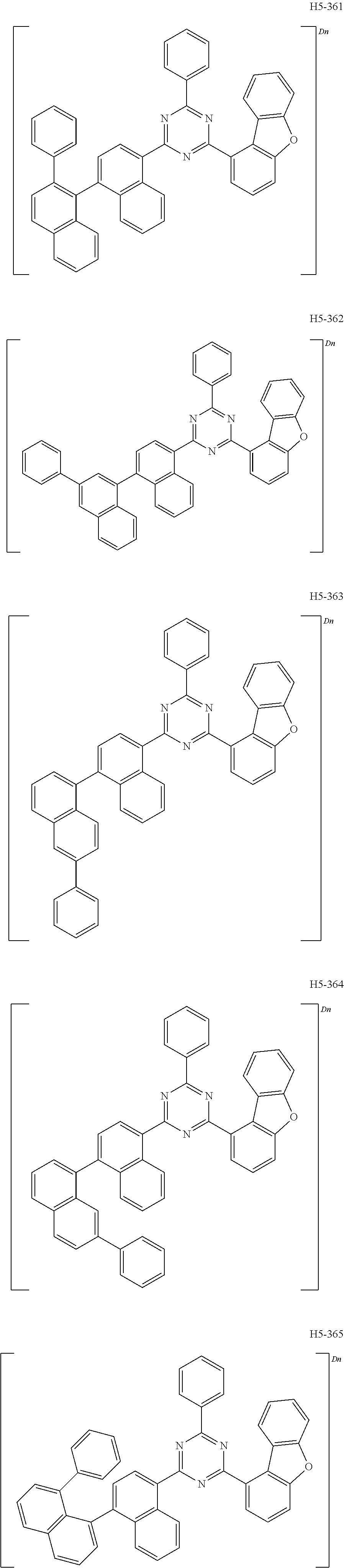 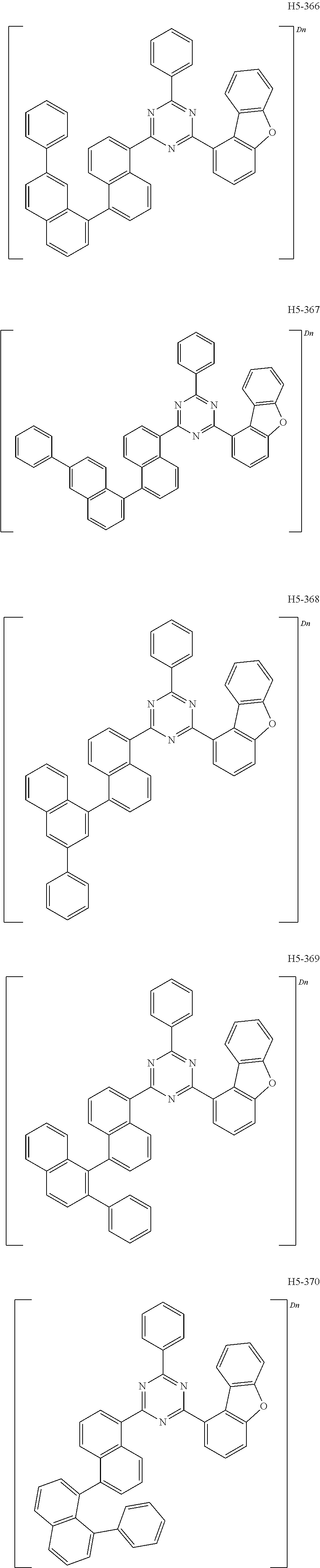 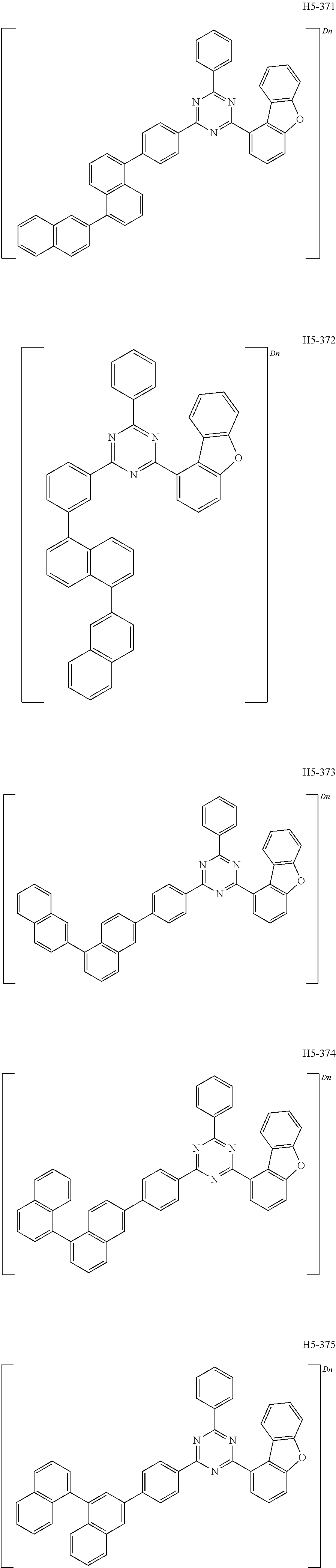 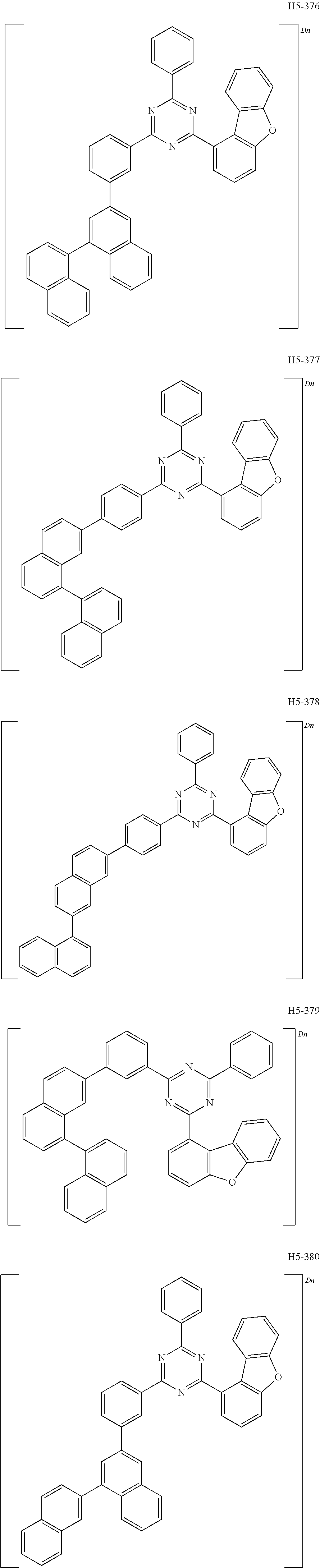 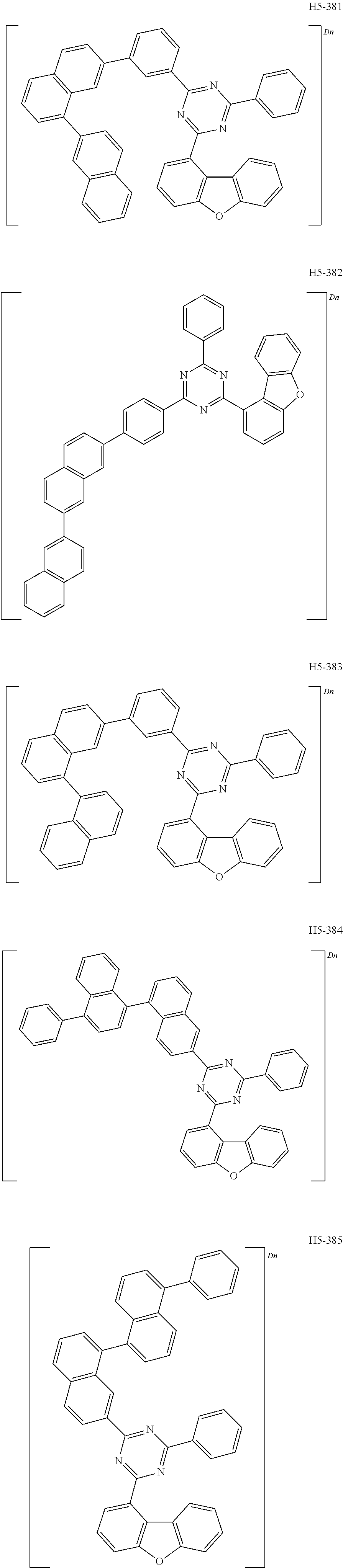 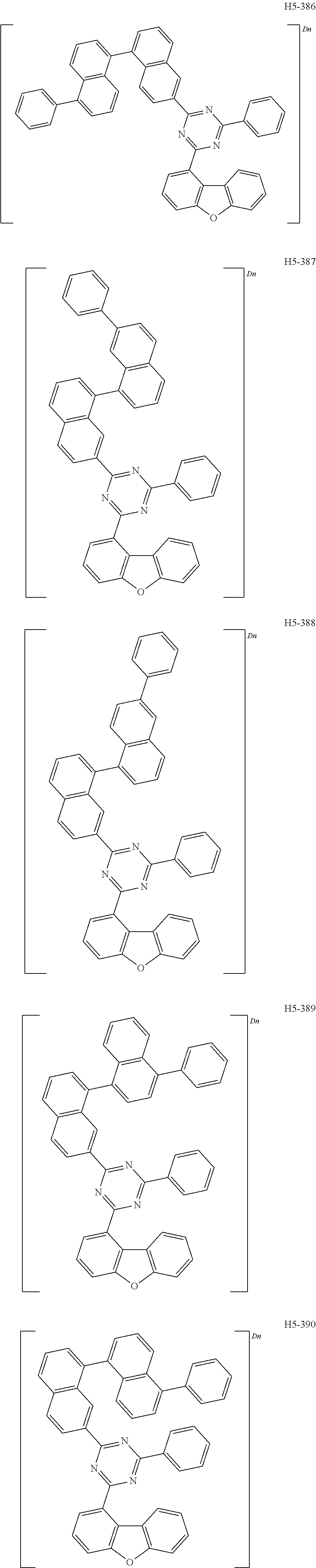 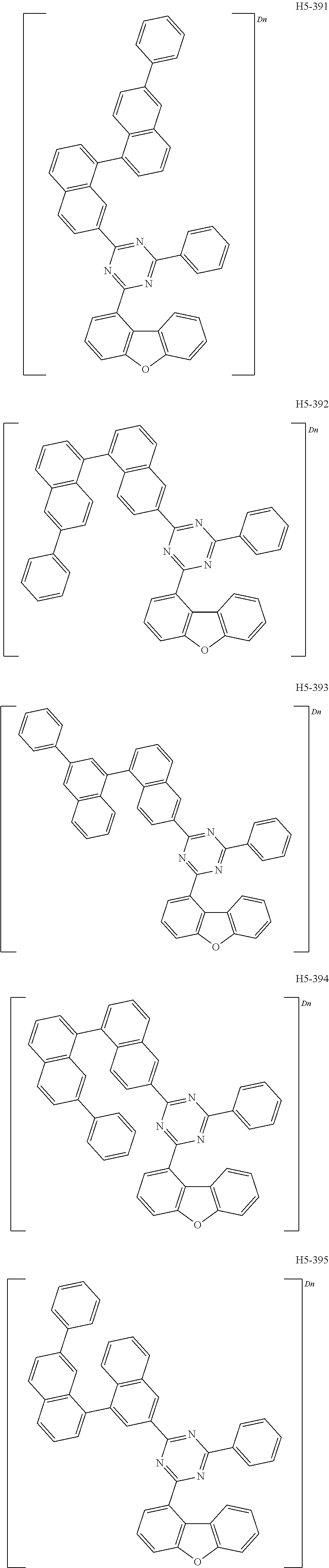 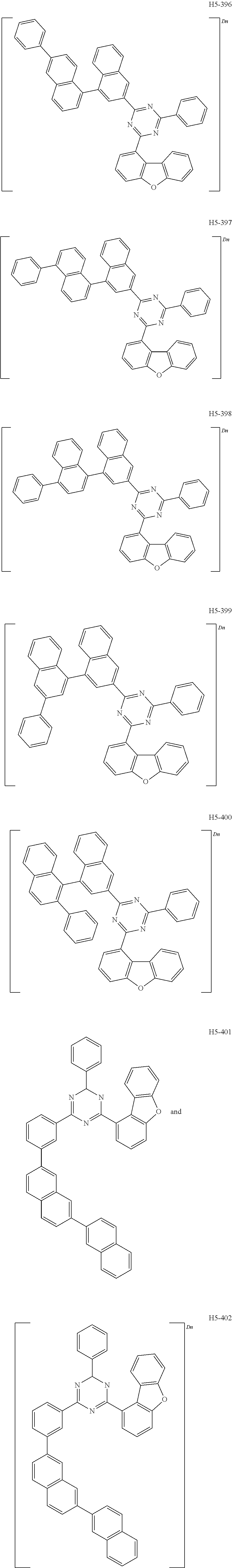
in the compounds above, Dₙ means that n number of hydrogens is replaced with deuterium, wherein n is an integer of 1 or more, and the upper limit of n is determined according to the number of hydrogens that may be substituted for each compound.

14. An organic electroluminescent device comprising an organic electroluminescent compound according to claim 12.
